(19) United States
(12) Patent Application Publication
(10) Pub. No.: US 2023/0374038 A1
(43) Pub. Date: Nov. 23, 2023

(54) MODULATORS OF CYSTIC FIBROSIS TRANSMEMBRANE CONDUCTANCE REGULATOR

(71) Applicant: VERTEX PHARMACEUTICALS INCORPORATED, Boston, MA (US)

(72) Inventors: Jason MCCARTNEY, Cardiff by the Sea, CA (US); Alexander Russell ABELA, Escondido, CA (US); Sunny ABRAHAM, San Diego, CA (US); Corey Don ANDERSON, Brighton, MA (US); Vijayalaksmi ARUMUGAM, San Marcos, CA (US); Jaclyn CHAU, San Diego, CA (US); Jeremy CLEMENS, San Diego, CA (US); Thomas CLEVELAND, San Marcos, CA (US); Timothy Richard COON, Carlsbad, CA (US); Timothy A. DWIGHT, Quincy, MA (US); Bryan A. FRIEMAN, La Jolla, CA (US); Peter GROOTENHUIS, Del Mar, CA (US); Sara Sabina HADIDA RUAH, La Jolla, CA (US); Yoshihiro ISHIHARA, San Diego, CA (US); Haripada KHATUYA, San Diego, CA (US); Paul KRENITSKY, San Francisco, CA (US); Mark Thomas MILLER, Rancho Santa Fe, CA (US); Prasuna PARASELLI, San Diego, CA (US); Fabrice PIERRE, La Jolla, CA (US); Alina SILINA, Needham, MA (US); Joe A. TRAN, San Marcos, CA (US); Jinglan ZHOU, San Diego, CA (US)

(21) Appl. No.: 18/248,068

(22) PCT Filed: Oct. 6, 2021
(86) PCT No.: PCT/US2021/053855

§ 371 Date: Apr. 6, 2023

Related U.S. Application Data
(60) Provisional application No. 63/088,759, filed on Oct. 7, 2020.

(51) Int. Cl. C07D 515/08; A61K 45/06; C07D 515/18; A61K 31/529; A61K 31/5377

(52) U.S. Cl. CPC C07D 515/08; A61K 45/06; C07D 515/18; A61K 31/529; A61K 31/5377

(57) ABSTRACT

This disclosure provides modulators of Cystic Fibrosis Transmembrane Conductance Regulator (CFTR), having the core structure: pharmaceutical compositions containing at least one such modulator, methods of treatment of mediated diseases, such as cystic fibrosis, using such modulators and pharmaceutical compositions, combination pharmaceutical compositions and therapies comprising such modulators, and processes and intermediates for making such modulators.

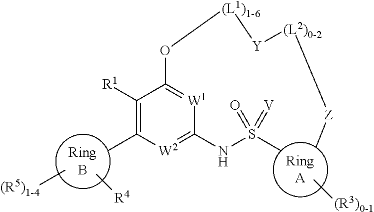

This application claims the benefit of priority of U.S. Provisional Application No. 63/088,759, filed Oct. 7, 2020, the contents of which are incorporated by reference herein in its entirety.

The disclosure relates to modulators of Cystic Fibrosis Transmembrane Conductance Regulator (CFTR), pharmaceutical compositions containing the modulators, methods of treatment of cystic fibrosis using such modulators and pharmaceutical compositions, combination therapies and combination pharmaceutical compositions employing such modulators, and processes and intermediates used for making such modulators.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 432 of these identified mutations, with sufficient evidence to define 352 mutations as disease causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease-causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺—K⁺-ATPase pump and Cl⁻ channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

A number of CFTR modulating compounds have recently been identified. However, compounds that can treat or reduce the severity of cystic fibrosis and other CFTR mediated diseases, and particularly the more severe forms of these diseases, are still needed.

One aspect of the disclosure provides novel compounds, including compounds of Formula I, compounds of any one of Formulae Ia, IIa, IIb, III, IV, V, VIa, and VIb, Compounds 1 to 369 (such as, e.g., Compounds 1-320, Compounds 321-330, Compounds 331-364, and Compounds 365-369), tautomers thereof, deuterated derivatives of those compounds and tautomers, and pharmaceutically acceptable salts of any of the foregoing.

Formula I encompasses compounds falling within the following structure:

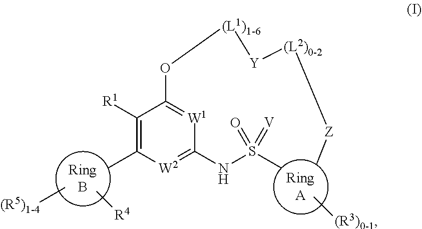

and includes tautomers of those compounds, deuterated derivatives of any of the compounds and tautomers, and pharmaceutically acceptable salts of any of the foregoing, wherein:
Ring A is selected from:
   C₆-C₁₀ aryl,
   C₃-C₁₀ cycloalkyl,
   3- to 10-membered heterocyclyl, and
   5- to 10-membered heteroaryl;
Ring B is selected from:
   C₆-C₁₀ aryl,
   C₃-C₁₀ cycloalkyl,
   3- to 10-membered heterocyclyl, and
   5- to 10-membered heteroaryl;
V is selected from O and NH;
W¹ is selected from N and CH;
W² is selected from N and CH, provided that at least one of W¹ and W² is N;
Y is selected from O, NR^{YN}, and C(R^{YC})₂;
Z is selected from O, NR^{ZN}, and C(R^{ZC})₂, provided that when L² is absent, either Y is C(R^{YC})₂ or Z is C(R^{ZC})₂;
each L¹ is independently selected from C(R^{L1})₂ and

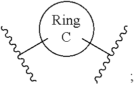

each L² is independently selected from C(R^{L2})₂;
Ring C is selected from C₆-C₁₀ aryl optionally substituted with 1-3 groups independently selected from:
   halogen,
   C₁-C₆ alkyl, and
   N(R^{N})₂;
R¹ is selected from:
   halogen,
   cyano,
   C₁-C₆ alkyl optionally substituted with 1-3 groups independently selected from hydroxyl, oxo, and N(R^{N})₂,
   C₁-C₆ alkoxy,
   C₁-C₆ fluoroalkyl,
   C₆-C₁₀ aryl optionally substituted with 1-3 groups independently selected from C₁-C₆ alkoxy,
   3- to 10-membered heterocyclyl optionally substituted with 1-3 groups independently selected from R^{N}, and
   5- to 10-membered heteroaryl optionally substituted with 1-3 groups independently selected from C₁-C₆ alkyl;
each R³ is independently selected from:
   halogen,
   C₁-C₆ alkyl,
   C₁-C₆ alkoxy,
   C₃-C₁₀ cycloalkyl,
   C₆-C₁₀ aryl optionally substituted with 1-3 groups independently selected from C₁-C₆ alkyl, and
   3- to 10-membered heterocyclyl;
R⁴ is selected from hydrogen and C₁-C₆ alkyl;
each R⁵ is independently selected from:
   hydrogen,
   halogen,
   hydroxyl,
   N(R^{N})₂,
   SO-Me,
   CH═C(R^{LC})₂, wherein both R^{LC} are taken together to form a C₃-C₁₀ cycloalkyl,
   C₁-C₆ alkyl optionally substituted with 1-3 groups independently selected from:
      hydroxyl,
      C₁-C₆ alkoxy optionally substituted with 1-3 groups independently selected from C₁-C₆ alkoxy and C₆-C₁₀ aryl,
      C₃-C₁₀ cycloalkyl,
      —(O)₀₋₁—(C₆-C₁₀ aryl) optionally substituted with 1-3 groups independently selected from C₁-C₆ alkyl and C₁-C₆ alkoxy,
      3- to 10-membered heterocyclyl, and
      N(R^{N})₂,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl,
   C₆-C₁₀ aryl, and
   3- to 10-membered heterocyclyl;
each R^{YN} and R^{ZN} is independently selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy, and
         COOH,
         N(R^{N})₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁-(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(R^{N})₂,
         C₁-C₆ alkyl (optionally substituted with 1-3 groups independently selected from oxo and C₁-C₆ alkoxy),
         C₁-C₆ alkoxy,
         C₁-C₆ fluoroalkyl,
         C₆-C₁₀ aryl optionally substituted with 1-3 groups independently selected from halogen, and
         5- to 10-membered heteroaryl, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3-SiMe₃), and N(R^{N})₂,
         C₁-C₆ alkoxy optionally substituted with 1-3 groups independently selected from hydroxyl, oxo, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from halogen and C₁-C₆ alkyl,
         —(O)₀₋₁—(C₆-C₁₀ aryl),
         —(O)₀₋₁-(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl) and
         5- to 10-membered heteroaryl optionally substituted with 1-4 groups independently selected from C₁-C₆ alkyl and C₃-C₁₀ cycloalkyl,
   C₁-C₆ fluoroalkyl,
   C₃-C₁₀ cycloalkyl optionally substituted with 1-3 groups independently selected from:
      hydroxyl,
      oxo,
      halogen,
      cyano,
      N(R^{N})₂,
      C₁-C₆ alkyl optionally substituted with 1-3 groups independently selected from:
         hydroxyl,
         oxo,
         N(R^{N})₂,
         C₁-C₆ alkoxy, and
         C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-member heterocyclyl optionally substituted with 1-3 groups independently selected from C₁-C₆ alkyl, and
      5- to 10-membered heteroaryl optionally substituted with 1-3 groups independently selected from:
         hydroxyl,
         cyano,
         oxo,
         halogen,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         C₆-C₁₀ aryl, and
         3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from halogen and C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl,
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
   R^{F},
each R^{YC} and R^{ZC} is independently selected from:
   hydrogen,
   C₁-C₆ alkyl optionally substituted with 1-3 groups independently selected from C₆-C₁₀ aryl (optionally substituted with 1-3 groups independently selected from C₁-C₆ alkyl),
   C₆-C₁₀ aryl optionally substituted with 1-3 groups independently selected from C₁-C₆ alkyl, and
   R^{F},
   or two R^{YC} are taken together to form an oxo group;
   or two R^{ZC} are taken together to form an oxo group;
each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl optionally substituted with 1-3 groups independently selected from C₁-C₆ alkyl (optionally substituted with 1-3 groups independently selected from hydroxyl and oxo),
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from:
         hydroxyl,
         oxo,
         cyano,
         SiMe₃,
         N(R^{N})₂, and
         C₃-C₁₀ cycloalkyl optionally substituted with 1-3 groups independently selected from C₁-C₆ fluoroalkyl,
      C₁-C₆ alkoxy optionally substituted with 1-3 groups independently selected from:
         C₃-C₁₀ cycloalkyl optionally substituted with 1-3 groups independently selected from C₁-C₆ fluoroalkyl, and
         C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl,
      3- to 10-membered heterocyclyl optionally substituted with 1-3 groups independently selected from C₁-C₆ alkyl, and
      5- to 10-membered heteroaryl,
   3- to 10-membered heterocyclyl optionally substituted with 1-3 groups independently selected from:
      C₁-C₆ alkyl optionally substituted with 1-3 groups independently selected from:
         oxo, and
         C₁-C₆ alkoxy,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl, and
   R^{F}.
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
   hydrogen,
   C₁-C₈ alkyl optionally substituted with 1-3 groups independently selected from:
      oxo,
      halogen,
      hydroxyl,
      NH₂,
      NHMe,
      NMe₂,
      C₁-C₆ alkoxy optionally substituted with 1-3 groups independently selected from C₆-C₁₀ aryl,
      —(O)₀₋₁—(C₃-C₁₀ cycloalkyl),
      C₆-C₁₀ aryl optionally substituted with 1-3 groups independently selected from halogen and C₁-C₆ alkyl,
      3- to 14-membered heterocyclyl optionally substituted with 1-4 groups independently selected from oxo and C₁-C₆ alkyl, and
      5- to 14-membered heteroaryl optionally substituted with 1-4 groups independently selected from oxo and C₁-C₆ alkyl,
   C₃-C₁₀ cycloalkyl optionally substituted with 1-3 groups independently selected from:
      hydroxyl,
      NH₂, and
      NHMe, and
      C₁-C₆ alkyl optionally substituted with 1-3 groups independently selected from hydroxyl,
   C₆-C₁₀ aryl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from:
   hydroxyl,
   oxo,
   cyano,
   C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
   C₁-C₆ alkoxy, and
   C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
   C₃-C₁₀ cycloalkyl optionally substituted with 1-3 groups independently selected from C₁-C₆ alkyl,
   C₆-C₁₀ aryl optionally substituted with 1-3 groups independently selected from:
      halogen,
      C₁-C₆ alkyl,
      N(R^{N})₂, and
      3- to 10-membered heterocyclyl optionally substituted with 1-3 groups independently selected from hydroxyl,
   3- to 11-membered heterocyclyl optionally substituted with 1-3 groups independently selected from:
      oxo,
      N(R^{N})₂,
      C₁-C₉ alkyl optionally substituted with 1-4 groups independently selected from:
         oxo,
         halogen,
         hydroxyl,
         N(R^{N})₂,
         —SO₂-(C₁-C₆ alkyl),
         C₁-C₆ alkoxy optionally substituted with 1-3 groups independently selected from halogen, C₆-C₁₀ aryl,
         C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N}, —O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₃-C₁₂ cycloalkyl optionally substituted with 1-4 groups independently selected from halogen, C₁-C₆ alkyl, and C₁-C₆ fluoroalkyl,
      C₆-C₁₀ aryl,
      3- to 10-membered heterocyclyl, and
      5- to 10-membered heteroaryl optionally substituted with 1-3 groups independently selected from C₁-C₆ alkoxy and C₁-C₆ fluoroalkyl, and
   5- to 12-membered heteroaryl optionally substituted with 1-3 groups independently selected from C₁-C₆ alkyl and C₁-C₆ fluoroalkyl.

Formula I also includes compounds of Formula Ia:

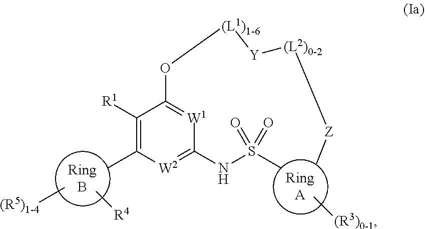

tautomers of those compounds, deuterated derivatives of any of the compounds and tautomers, and pharmaceutically acceptable salts of any of the foregoing, wherein Ring A, Ring B, W¹, W², Y, Z, L¹, L², R¹, R³, R⁴, and R⁵ are as defined for Formula I.

Formula I also includes compounds of Formula IIa:

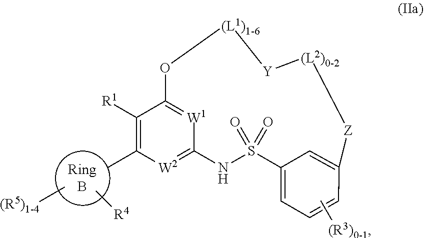

tautomers of those compounds, deuterated derivatives of any of the compounds and tautomers, and pharmaceutically acceptable salts of any of the foregoing, wherein Ring B, W¹, W², Y, Z, L¹, L², R¹, R³, R⁴, and R⁵ are as defined for Formula I.

Formula I also includes compounds of Formula IIb:

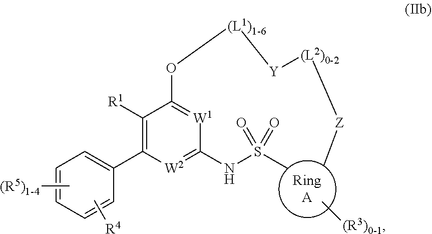

tautomers of those compounds, deuterated derivatives of any of the compounds and tautomers, and pharmaceutically acceptable salts of any of the foregoing, wherein Ring A, W¹, W², Y, Z, L¹, L², R¹, R³, R⁴, and R⁵ are as defined for Formula I.

Formula I also includes compounds of Formula III:

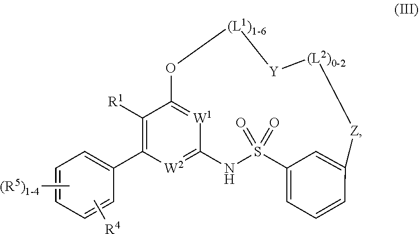

tautomers of those compounds, deuterated derivatives of any of the compounds and tautomers, and pharmaceutically acceptable salts of any of the foregoing, wherein W¹, W², Y, Z, L¹, L², R¹, R⁴, and R⁵ are as defined for Formula I.

Formula I also includes compounds of Formula IV:

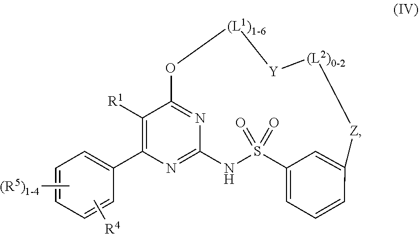

tautomers of those compounds, deuterated derivatives of any of the compounds and tautomers, and pharmaceutically acceptable salts of any of the foregoing, wherein Y, Z, L¹, L², R¹, R⁴, and R⁵ are as defined for Formula I.

Formula I also includes compounds of Formula V:

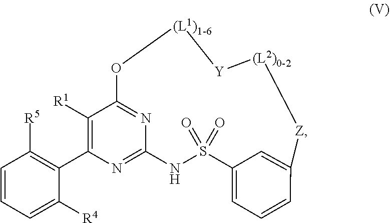

tautomers of those compounds, deuterated derivatives of any of the compounds and tautomers, and pharmaceutically acceptable salts of any of the foregoing, wherein Y, Z, L¹, L², R¹, R⁴, and R⁵ are as defined for Formula I.

Formula I also includes compounds of Formula VIa and Formula VIb:

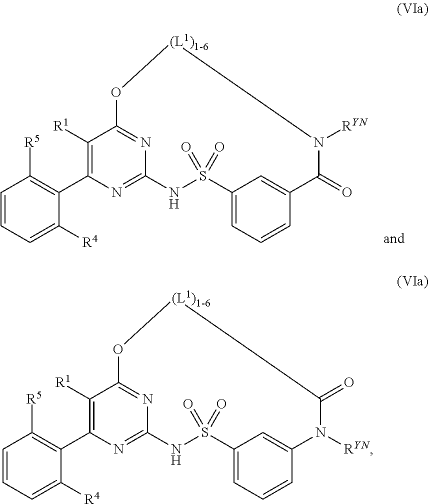

tautomers of those compounds, deuterated derivatives of any of the compounds and tautomers, and pharmaceutically acceptable salts of any of the foregoing, wherein L¹, R¹, R⁴, R⁵, R^{YN}, and R^{ZN} are as defined for Formula I.

Another aspect of the disclosure provides pharmaceutical compositions comprising at least one compound chosen from the novel compounds disclosed herein, deuterated derivatives thereof, and pharmaceutically acceptable salts of any of the foregoing, and at least one pharmaceutically acceptable carrier, which compositions may further include at least one additional active pharmaceutical ingredient. In some embodiments of the pharmaceutical compositions disclosed herein, the at least one additional active pharmaceutical ingredient is at least one other CFTR modulator. In some embodiments, the at least one other CFTR modulator is selected from CFTR potentiators and CFTR modulators. Thus, another aspect of the disclosure provides methods of treating the CFTR-mediated disease, including cystic fibrosis, comprising administering at least one of compound chosen from the novel compounds disclosed herein, deuterated derivatives thereof, and pharmaceutically acceptable salts of any of the foregoing, and at least one pharmaceutically acceptable carrier, optionally as part of a pharmaceutical composition comprising at least one additional component, to a subject in need thereof. In some embodiments, the at least one additional active pharmaceutical ingredient in the methods of treating disclosed herein is at least one other CFTR modulator. In some embodiments, the at least one other CFTR modulator is selected from CFTR potentiators, and CFTR modulators.

In certain embodiments, the pharmaceutical compositions of the disclosure comprise at least one (i.e., one or more) compound(s) chosen from compounds of Formula I, compounds of any one of Formulae Ia, IIa, IIb, III, IV, V, VIa, V, VIa, and VIb, Compounds 1 to 369 (such as, e.g., Compounds 1-320, Compounds 321-330, Compounds 331-364, and Compounds 365-369), tautomers thereof, deuterated derivatives of those compounds and tautomers, and pharmaceutically acceptable salts of any of the foregoing. In some embodiments, compositions comprising at least one compound chosen from compounds of Formula I, compounds of any one of Formulae Ia, IIa, IIb, III, IV, V, VIa, and VIb, Compounds 1 to 369 (such as, e.g., Compounds 1-320, Compounds 321-330, Compounds 331-364, and Compounds 365-369), tautomers thereof, deuterated derivatives of those compounds and tautomers, and pharmaceutically acceptable salts of any of the foregoing may optionally further comprise (a) at least one (i.e., one or more) compound(s) chosen from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (tezacaftor), 3-(6-(1-(2,2-difluorobenzo[d][1,3]dioxol-5-yl)cyclopropane carboxamido)-3-methylpyridin-2-yl)benzoic acid (lumacaftor) and deuterated derivatives and pharmaceutically acceptable salts of tezacaftor and lumacaftor; and/or (b) at least one (i.e., one or more) compound(s) chosen from N-[2,4-bis(1,1-dimethylethyl)-5-hydroxyphenyl]-1,4-dihydro-4-oxoquinoline-3-carboxamide (ivacaftor) or N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (deutivacaftor) and pharmaceutically acceptable salts thereof, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

Another aspect of the disclosure provides methods of treating the CFTR-mediated disease cystic fibrosis comprising administering to a patient in need thereof at least one compound chosen from the novel compounds disclosed herein, pharmaceutically acceptable salts thereof, and deuterated derivatives of any of the foregoing, and optionally further administering one or more additional CFTR modulating agents. A further aspect of the disclosure provides compounds of Formula I, compounds of any one of Formulae Ia, IIa, IIb, III, IV, V, VIa, and VIb, Compounds 1 to 369 (such as, e.g., Compounds 1-320, Compounds 321-330, Compounds 331-364, and Compounds 365-369), tautomers thereof, deuterated derivatives of those compounds and tautomers, and pharmaceutically acceptable salts of any of the foregoing) and pharmaceutical compositions comprising those compounds and, optionally further comprising one or more CFTR modulating agents, for use in therapy or for use in the manufacture of a medicament. In some embodiments the one or more additional CFTR modulating agents are selected from CFTR potentiators. In some embodiments, the one or more additional CFTR modulating agents are selected from CFTR correctors. In some embodiments, the one or more additional CFTR modulating agents include both a potentiator and a corrector. In some embodiments the one or more additional CFTR modulating agents are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

A further aspect of the disclosure provides intermediates and methods for making the compounds and compositions disclosed herein.

### Definitions

“Tezacaftor” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

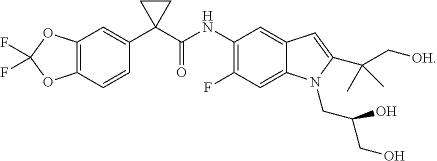 [SMILES: CC(C)(CO)c1cc2cc(NC(=O)C3(c4ccc5c(c4)OC(F)(F)O5)CC3)c(F)cc2n1C[C@@H](O)CO]

Tezacaftor may be in the form of a deuterated derivative, a pharmaceutically acceptable salt, or a pharmaceutically acceptable salt of a deuterated derivative. Tezacaftor and methods of making and using tezacaftor are disclosed in WO 2010/053471, WO 2011/119984, WO 2011/133751, WO 2011/133951, WO 2015/160787, and US 2009/0131492, each of which is incorporated hereing by reference.

“Ivacaftor” as used throughout this disclosure refers to N-[2,4-bis(1,1-dimethylethyl)-5-hydroxyphenyl]-1,4-dihydro-4-oxoquinoline-3-carboxamide, which is depicted by the structure:

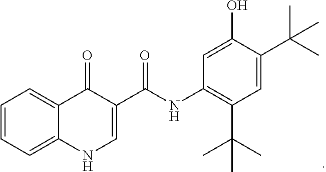 [SMILES: CC(C)(C)c1cc(C(C)(C)C)c(NC(=O)c2c[nH]c3ccccc3c2=O)cc1O]

Ivacaftor may also be in the form of deuterated derivative, a pharmaceutically acceptable salt, or a pharmaceutically acceptable salt of a deuterated derivative. Ivacaftor and methods of making and using ivacaftor are disclosed in WO 2006/002421, WO 2007/079139, WO 2010/108162, and WO 2010/019239, each of which is incorporated herein by reference.

In some embodiments, a deuterated derivative of ivacaftor (deutivacaftor) is employed in the compositions and methods disclosed herein. A chemical name for deutivacaftor is N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide, as depicted by the structure:

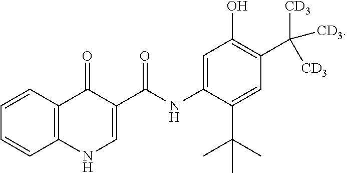 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Deutivacaftor may be in the form of further deuterated derivative, a pharmaceutically acceptable salt, or a pharmaceutically acceptable salt of a deuterated derivative. Deutivacaftor and methods of making and using deutivacaftor are disclosed in WO 2012/158885, WO 2014/078842, and U.S. Pat. No. 8,865,902, each of which is incorporated herein by reference.

“Lumacaftor” as used herein, refers to 3-(6-(1-(2,2-difluorobenzo[d][1,3]dioxol-5-yl)cyclopropanecarboxamido)-3-methylpyridin-2-yl)benzoic acid, which is depicted by the chemical structure:

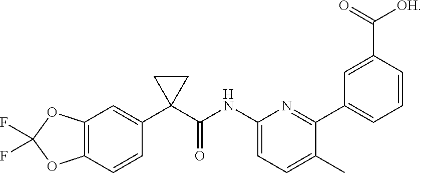 [SMILES: Cc1ccc(NC(=O)C2(c3ccc4c(c3)OC(F)(F)O4)CC2)nc1-c1cccc(C(=O)O)c1]

Lumacaftor may be in the form of a deuterated derivative, a pharmaceutically acceptable salt, or a pharmaceutically acceptable salt of a deuterated derivative. Lumacaftor and methods of making and using Lumacaftor are disclosed in WO 2007/056341, WO 2009/073757, and WO 2009/076142, incorporated herein by reference.

As used herein, the term “alkyl” refers to a saturated or partially saturated, branched or unbranched aliphatic hydrocarbon containing carbon atoms (such as, for example, 1, 2, 3, 4, 5, 6, 7, 8, 9, 10, 11, 12, 13, 14, 15, 16, 17, 18, 19, or 20 carbon atoms), in which one or more adjacent carbon atoms may be interrupted by a double (alkenyl) bond or triple (alkynyl) bond. Alkyl groups may be substituted or unsubstituted.

As used herein, the term “haloalkyl group” refers to an alkyl group substituted with one or more halogen atoms, e.g., fluoroalkyl, which refers to an alkyl group substituted with one or more fluorine atoms.

The term “alkoxy,” as used herein, refers to an alkyl or cycloalkyl covalently bonded to an oxygen atom. Alkoxy groups may be substituted or unsubstituted.

As used herein, the term “haloalkoxyl group” refers to an alkoxy group substituted with one or more halogen atoms.

As used herein, “cycloalkyl” refers to a cyclic, bicyclic, tricyclic, or polycyclic non-aromatic hydrocarbon groups having 3 to 12 carbons (such as, for example 3-10 carbons) and may include one or more unsaturated bonds. “Cycloalkyl” groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbornyl, dispiro[2.0.2.1]heptane, and spiro[2,3]hexane. Cycloalkyl groups may be substituted or unsubstituted.

The term “aryl,” as used herein, is a functional group or substituent derived from an aromatic ring and encompasses monocyclic aromatic rings and bicyclic, tricyclic, and fused ring systems wherein at least one ring in the system is aromatic. Non-limiting examples of aryl groups include phenyl, naphthyl, and 1,2,3,4-tetrahydronaphthalenyl.

The term “heteroaryl ring,” as used herein, refers to an aromatic ring comprising at least one ring atom that is a heteroatom, such as O, N, or S. Heteroaryl groups encompass monocyclic rings and bicyclic, tricyclic, bridged, fused, and spiro ring systems (including mono spiro and dispiro rings) wherein at least one ring in the system is aromatic. Non-limiting examples of heteroaryl rings include pyridine, quinoline, indole, and indoline.

As used herein, the term “heterocyclyl ring” refers to a non-aromatic hydrocarbon containing 3 to 12 atoms in a ring (such as, for example, 3-10 atoms) comprising at least one ring atom that is a heteroatom, such as O, N, or S, and may include one or more unsaturated bonds. “Heterocyclyl” rings encompass monocyclic, bicyclic, tricyclic, polycyclic, bridged, fused, and spiro rings, including mono spiro and dispiro rings.

“Substituted,” whether preceded by the term “optionally” or not, indicates that at least one hydrogen of the “substituted” group is replaced by a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position.

Non-limiting examples of protecting groups for nitrogen include, for example, t-butyl carbamate (Boc), benzyl (Bn), para-methoxybenzyl (PMB), tetrahydropyranyl (THP), 9-fluorenylmethyl carbamate (Fmoc), benzyl carbamate (Cbz), methyl carbamate, ethyl carbamate, 2,2,2-trichloroethyl carbamate (Troc), 2-trimethylsilylethyl carbamate (Teoc), allyl carbamate (Aloc or Alloc), formamide, acetamide, benzamide, allylamine, trifluoroacetamide, triphenylmethylamine, benzylideneamine, and p-toluenesulfonamide. A comprehensive list of nitrogen protecting groups can be found in Wuts, P. G. M. “Greene's Protective Groups in Organic Synthesis: Fifth Edition,” 2014, John Wiley and Sons.

As used herein, “deuterated derivative(s)” refers to a compound having the same chemical structure as a reference compound, with one or more hydrogen atoms replaced by a deuterium atom. In chemical structures, deuterium is represented as “D.” In some embodiments, the one or more hydrogens replaced by deuterium are part of an alkyl group. In some embodiments, the one or more hydrogens replaced by deuterium are part of a methyl group.

The phrase “and deuterated derivatives and pharmaceutically acceptable salts thereof” is used interchangeably with “and deuterated derivatives and pharmaceutically acceptable salts thereof of any of the forgoing” in reference to one or more specified compounds. These terms, as used herein are intended to include deuterated derivatives of the specified compound or compounds and pharmaceutically acceptable salts of the specified compound or compounds, as well as pharmaceutically acceptable salts of deuterated derivatives of the specified compound or compounds.

As used herein, “CFTR” means cystic fibrosis transmembrane conductance regulator.

As used herein, the term “CFTR modulator” refers to a compound that increases the activity of CFTR. The increase in activity resulting from a CFTR modulator includes but is not limited to compounds that correct, potentiate, stabilize and/or amplify CFTR.

As used herein, the terms “corrector” and “CFTR corrector” are used interchangeably herein, and refer to a compound that facilitates the processing and trafficking of CFTR to increase the amount of CFTR at the cell surface. The novel compounds disclosed herein are CFTR correctors. Tezacaftor and lumacaftor, and deuterated derivatives and pharmaceutically acceptable salts thereof as referenced herein, are correctors. Thus, in some embodiments, a combination of at least one compound selected from compounds of Formula I, compounds of any one of Formulae Ia, IIa, IIb, III, IV, V, VIa, and VIb, Compounds 1-369, tautomers thereof, deuterated derivatives of those compounds and tautomers, and pharmaceutically acceptable salts of any of the foregoing, and other specified CFTR modulating agents, will include another CFTR corrector, such as, e.g., a corrector compound selected from tezacaftor, lumacaftor, and deuterated derivatives and pharmaceutically acceptable salts thereof.

As used herein, the terms “potentiator” and “CFTR potentiator” refer to a compound that increases the channel activity of CFTR protein located at the cell surface, resulting in enhanced ion transport. Ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof as referenced herein are CFTR potentiators. Thus, in some embodiments, a combination of at least one compound selected from compounds of Formula I, compounds of any one of Formulae Ia, IIa, IIb, III, IV, V, VIa, and VIb, Compounds 1-369, tautomers thereof, deuterated derivatives of those compounds and tautomers, and pharmaceutically acceptable salts of any of the foregoing, and other specified CFTR modulating agents, will include a CFTR potentiator, such as, e.g., ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof.

It will be appreciated that when a description of a combination of compound selected from compounds of Formula I, compounds of any one of Formulae Ia, IIa, IIb, III, IV, V, VIa, and VIb, Compounds 1-369, tautomers thereof, deuterated derivatives of those compounds and tautomers, and pharmaceutically acceptable salts of any of the foregoing, and other specified CFTR modulating agents is provided herein, the combination will typically but not necessarily include a CFTR potentiator, such as, e.g., ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, or a deuterated derivative or pharmaceutically acceptable salt of any of the foregoing. Typically, but not necessarily, a reference to one of the modulators disclosed herein, in combination with other CFTR modulators will include only one CFTR potentiator. In some embodiments a combination of at least one compound disclosed herein and other specified CFTR modulating agents, will include both a CFTR corrector and a CFTR potentiator.

The term “at least one compound selected from, as used herein, refers to the selection of one or more of the compounds from a specified group.

A reference to “Compounds 1-369 in this disclosure is intended to represent a reference to each of Compounds 1 through 369 individually as well as a reference to groups of compounds, such as, e.g., Compounds 1-220, Compounds 321-330, Compounds 331-364, and Compounds 365-369.

As used herein, the term “active pharmaceutical ingredient” or “therapeutic agent” (“API”) refers to a biologically active compound.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement in one or more symptoms of CF or lessening the severity of CF or one or more symptoms of CF in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

It should be understood that references herein to methods of treatment (e.g., methods of treating a CFTR mediated disease or a method of treating cystic fibrosis) using one or more compounds of the invention optionally in combination with one or more additional CFTR modulating agents (e.g., compound chosen from compounds of Formula I, compounds of any one of Formulae Ia, IIa, IIb, III, IV, V, VIa, and VIb, Compounds 1-369, tautomers thereof, deuterated derivatives of those compounds and tautomers, and pharmaceutically acceptable salts of any of the foregoing, optionally in combination with one or more additional CFTR modulating agents) should also be interpreted as references to:
one or more compounds (e.g., compound chosen from compounds of Formula I, compounds of any one of Formulae Ia, IIa, IIb, III, IV, V, VIa, and VIb, Compounds 1-369, tautomers thereof, deuterated derivatives of those compounds and tautomers, and pharmaceutically acceptable salts of any of the foregoing, optionally in combination with one or more additional CFTR modulating agents) for use in methods of treating, e.g., cystic fibrosis optionally in combination with one or more additional CFTR modulating agents; and/or
the use of one or more compounds (e.g., compound chosen from compounds of Formula I, compounds of any one of Formulae Ia, IIa, IIb, III, IV, V, VIa, VIb, VIIa, and VIIb, Compounds 1-369, tautomers thereof, deuterated derivatives of those compounds and tautomers, and pharmaceutically acceptable salts of any of the foregoing, optionally in combination with one or more additional CFTR modulating agents) in the manufacture of a medicament for treating, e.g., cystic fibrosis.

It should be also understood that references herein to methods of treatment (e.g., methods of treating a CFTR mediated disease or a method of treating cystic fibrosis) using a pharmaceutical composition of the invention (e.g., a pharmaceutical composition comprising at least one compound chosen from compounds of Formula I, compounds of any one of Formulae Ia, IIa, IIb, III, IV, V, VIa, VIb, VIIa, and VIIb, Compounds 1-369, tautomers thereof, deuterated derivatives of those compounds and tautomers, and pharmaceutically acceptable salts of any of the foregoing and optionally further comprising one or more additional CFTR modulating agents) should also be interpreted as references to:
a pharmaceutical composition (e.g., a pharmaceutical composition comprising at least one compound chosen from compounds of Formula I, compounds of any one of Formulae Ia, IIa, IIb, III, IV, V, VIa, VIb, VIIa, and VIIb, Compounds 1-369, tautomers thereof, deuterated derivatives of those compounds and tautomers, and pharmaceutically acceptable salts of any of the foregoing and optionally further comprising one or more additional CFTR modulating agents) for use in methods of treating, e.g., cystic fibrosis; and/or
the use of a pharmaceutical composition (e.g., a pharmaceutical composition comprising at least one compound chosen from compounds of Formula I, compounds of any one of Formulae Ia, IIa, IIb, III, IV, V, VIa, VIb, VIIa, and VIIb, Compounds 1-369, tautomers thereof, deuterated derivatives of those compounds and tautomers, and pharmaceutically acceptable salts of any of the foregoing and optionally further comprising one or more additional CFTR modulating agents) in the manufacture of a medicament for treating, e.g., cystic fibrosis.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

The terms “about” and “approximately” may refer to an acceptable error for a particular value as determined by one of skill in the art, which depends in part on how the values is measured or determined. In some embodiments, the terms “about” and “approximately” mean within 20%, 15%, 10%, 5%, 4%, 3%, 2%, 1%, or 0.5% of a given value or range.

As used herein, the term “solvent” refers to any liquid in which the product is at least partially soluble (solubility of product >1 g/l).

As used herein, the term “room temperature” or “ambient temperature” means 15° C. to 30° C.

It will be appreciated that certain compounds of this disclosure may exist as separate stereoisomers or enantiomers and/or mixtures of those stereoisomers or enantiomers.

Certain compounds disclosed herein may exist as tautomers and both tautomeric forms are intended, even though only a single tautomeric structure is depicted. For example, a description of Compound X is understood to include its tautomer Compound Y and vice versa, as well as mixtures thereof.

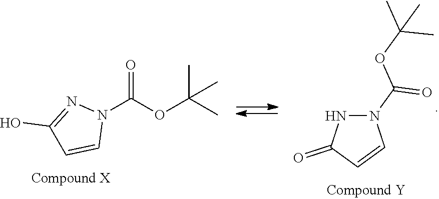

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance.

As used herein, the term “pharmaceutically acceptable salt” refers to a salt form of a compound of this disclosure wherein the salt is nontoxic. Pharmaceutically acceptable salts of the compounds of this disclosure include those derived from suitable inorganic and organic acids and bases. A “free base” form of a compound, for example, does not contain an ionically bonded salt.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

### Methods of Treatment

Any of the novel compounds disclosed herein, such as for example, compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, can act as a CFTR modulator, i.e., it modulates CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the disclosure provides methods of treating, lessening the severity of, or symptomatically treating cystic fibrosis in a patient comprising administering to the patient an effective amount of any of the novel compounds disclosed herein, such as for example, compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, alone or in combination with another active ingredient, such as one or more additional CFTR modulating agents. In some embodiments, the one (or more) CFTR modulating agent is a corrector. In some embodiments, the one (or more) CFTR modulating agent is a potentiator. In some embodiments, the CFTR modulating agents include both a corrector and a potentiator. In some embodiments, the one or more CFTR modulating agents are selected from potentiators: ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing; and correctors: lumacaftor, tezacaftor, and deuterated derivatives and pharmaceutically acceptable salts thereof.

In some embodiments, 5 mg to 500 mg of a compound disclosed herein, a tautomer thereof, a deuterated derivative of the compound and tautomer, or a pharmaceutically acceptable salt of any of the foregoing are administered daily.

In some embodiments, the patient has an F508del/minimal function (MF) genotype, F508del/F508del genotype (homozygous for the F508del mutation), F508del/gating genotype, or F508del/residual function (RF) genotype. In some embodiments the patient is heterozygous and has one F508del mutation. In some embodiments the patient is homozygous for the N1303K mutation.

In some embodiments, the patient has at least one F508del mutation in the CFTR gene. In some embodiments, the patient has a CFTR gene mutation that is responsive to a compound, tautomer, deutrated derivative, or pharmaceutically acceptable salt of the disclosure based on in vitro data. In some embodiments, the patient is heterozygous and has an F508del mutation on one allele and a mutation on the other allele selected from Table 2:

TABLE 2
CFTR Mutations

| MF Category | Mutation | | | | |
|---|---|---|---|---|---|
| Nonsense mutations | Q2X | L218X | Q525X | R792X | E1104X |
| | S4X | Q220X | G542X | E822X | W1145X |
| | W19X | Y275X | G550X | W882X | R1158X |
| | G27X | C276X | Q552X | W846X | R1162X |
| | Q39X | Q290X | R553X | Y849X | S1196X |
| | W57X | G330X | E585X | R851X | W1204X |
| | E60X | W401X | G673X | Q890X | L1254X |
| | R75X | Q414X | Q685X | S912X | S1255X |
| | L88X | S434X | R709X | Y913X | W1282X |
| | E92X | S466X | K710X | Q1042X | Q1313X |
| | Q98X | S489X | Q715X | W1089X | Q1330X |
| | Y122X | Q493X | L732X | Y1092X | E1371X |
| | E193X | W496X | R764X | W1098X | Q1382X |
| | W216X | C524X | R785X | R1102X | Q1411X |
| Canonical splice | 185 + 1G→T | 711 + 5G→A | 1717 − 8G→A | 2622 + 1G→A | 3121 − 1G→A |
| mutations | 296 + 1G→A | 712 − 1G→T | 1717 − 1G→A | 2790 − 1G→C | 3500 − 2A→G |
| | 296 + 1G→T | 1248 + 1G→A | 1811 + 1G→C | 3040G→C | 3600 + 2insT |
| | 405 + 1G→A | 1249 − 1G→A | 1811 + 1.6kbA→G | (G970R) | 3850 − 1G→A |
| | 405 + 3A→C | 1341 + 1G→A | 1811 + 1643G→T | 3120G→A | 4005 + 1G→A |
| | 406 − 1G→A | 1525 − 2A→G | 1812 − 1G→A | 3120 + 1G→A | 4374 + 1G→T |
| | 621 + 1G→T | 1525 − 1G→A | 1898 + 1G→A | 3121 − 2A→G | |
| | 711 + 1G→T | | 1898 + 1G→C | | |
| Small (≤3 nucleotide) | 182delT | 1078delT | 1677delTA | 2711delT | 3737delA |
| insertion/deletion | 306insA | 1119delA | 1782delA | 2732insA | 3791delC |
| (ins/del) frameshift | 306delTAGA | 1138insG | 1824delA | 2869insG | 3821delT |
| mutations | 365-366insT | 1154insTC | 1833delT | 2896insAG | 3876delA |
| | 394delTT | 1161delC | 2043delG | 2942insT | 3878delG |
| | 442delA | 1213delT | 2143delT | 2957delT | 3905insT |
| | 444delA | 1259insA | 2183AA→G^{a} | 3007delG | 4016insT |
| | 457TAT→G | 1288insTA | 2184delA | 3028delA | 4021dupT |
| | 541delC | 1343delG | 2184insA | 3171delC | 4022insT |
| | 574delA | 1471delA | 2307insA | 3171insC | 4040delA |
| | 663delT | 1497delGG | 2347delG | 3271delGG | 4279insA |
| | 849delG | 1548delG | 2585delT | 3349insT | 4326delTC |
| | 935delA | 1609del CA | 2594delGT | 3659delC | |
| Non-small (>3 | CFTRdele1 | CFTRdele16-17b | | 1461ins4 | |
| nucleotide) | CFTRdele2 | CFTRdele17a,17b | | 1924del7 | |
| insertion/deletion | CFTRdele2,3 | CFTRdele17a-18 | | 2055del9→A | |
| (ins/del) frameshift | CFTRdele2-4 | CFTRdele19 | | 2105-2117del13insAGAAA | |
| mutations | CFTRdele3-10,14b-16 | CFTRdele19-21 | | 2372del8 | |
| | CFTRdele4-7 | CFTRdele21 | | 2721del11 | |
| | CFTRdele4-11 | CFTRdele22-24 | | 2991del32 | |
| | CFTR50kbdel | CFTRdele22,23 | | 3667ins4 | |
| | CFTRdup6b-10 | 124del23bp | | 4010del4 | |
| | CFTRdele11 | 602del14 | | 4209TGTT→AA | |
| | CFTRdele13,14a | 852del22 | | | |
| | CFTRdele14b-17b | 991del5 | | | |
| Missense mutations that | A46D | V520F | Y569D | N1303K | |
| Are not responsive in | G85E | A559T | L1065P | | |
| vitro to TEZ, IVA, or | R347P | R560T | R1066C | | |
| TEZ/IVA | L467P | R560S | L1077P | | |
| and | I507del | A561E | M1101K | | |
| % PI >50% and | | | | | |
| SwCl⁻ >86 mmol/L | | | | | |

^{a}Also known as 2183delAA→G.
CFTR: cystic fibrosis transmembrane conductance regulator;
IVA: ivacaftor.
SwCl: sweat chloride.
TEZ: tezacaftor
Source: CFTR2.org [Internet]. Baltimore (MD): Clinical and functional translation of CFTR. The Clinical and Functional Translation of CFTR (CFTR2), US Cystic Fibrosis Foundation, Johns Hopkins University, the Hospital for Sick Children. Available at: http://www.cftr2.org/. Accessed 15 May 2018.
Notes:
% PI: percentage of F508del-CFTR heterozygous patients in the CFTR2 patient registry who are pancreatic insufficient; SwCl: mean sweat chloride of F508del-CVTR heterozygous patients in the CFTR2 patient registry.

In some embodiments, the disclosure also is directed to methods of treatment using isotope-labelled compounds of the afore-mentioned compounds, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P ³⁵S, ¹⁸F and ³⁶Cl, respectively.

The isotope-labelled compounds and salts can be used in a number of beneficial ways. They can be suitable for medicaments and/or various types of assays, such as substrate tissue distribution assays. For example, tritium (³H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (²H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Combination Therapies

One aspect disclosed herein provides methods of treating cystic fibrosis and other CFTR mediated diseases using any of the novel compounds disclosed herein, such as for example, compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, in combination with at least one additional active pharmaceutical ingredient.

In some embodiments, at least one additional active pharmaceutical ingredient is selected from mucolytic agents, bronchodilators, antibiotics, anti-infective agents, and anti-inflammatory agents.

In some embodiments, the additional therapeutic agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In some embodiments, the additional agent is a mucolyte. Exemplary mucolytes useful herein includes Pulmozyme®.

In some embodiments, the additional agent is a bronchodilator. Exemplary bronchodiltors include albuterol, metaprotenerol sulfate, pirbuterol acetate, salmeterol, or tetrabuline sulfate.

In some embodiments, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simavastatin.

In some embodiments, the additional agent is a nutritional agent. Exemplary nutritional agents include pancrelipase (pancreating enzyme replacement), including Pancrease®, Pancreacarb®, Ultrase®, or Creon®, Liprotomase® (formerly Trizytek®), Aquadeks®, or glutathione inhalation. In one embodiment, the additional nutritional agent is pancrelipase.

In some embodiments, at least one additional active pharmaceutical ingredient is selected from CFTR modulating agents. In some embodiments, the additional active pharmaceutical ingredient is selected from CFTR potentiators. In some embodiments, the potentiator is selected from ivacaftor, deutivacaftor, and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. In some embodiments, the additional active pharmaceutical ingredient is chosen from CFTR correctors. In some embodiments, the correctors are selected from lumacaftor, tezacaftor, and derivatives and pharmaceutically acceptable salts of any of the foregoing.

In some embodiments, the at least one additional active pharmaceutical ingredient is chosen from (a) tezacaftor, lumacaftor, and deuterated derivatives and pharmaceutically acceptable salts thereof; and/or (b) ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. In some embodiments, the combination therapies provided herein comprise (a) a compound selected from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, (b) at least one compound selected from tezacaftor, lumacaftor, and deuterated derivatives and pharmaceutically acceptable salts thereof; or (c) at least one compound selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. In some embodiments, the combination therapies provided herein comprise (a) at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof; and (c) at least one compound selected from ivacaftor or deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof.

In some embodiments, at least one compound chosen from compounds of compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof. In some embodiments, at least one compound chosen from compounds of compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in combination with at least one compound chosen from ivacaftor and deuterated derivatives and pharmaceutically acceptable salts thereof. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in combination with at least one compound chosen from deutivacaftor and deuterated derivatives and pharmaceutically acceptable salts thereof. In some embodiments, at least one compound chosen from compounds of compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in combination with at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof.

In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

Each of the compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, independently can be administered once daily, twice daily, or three times daily. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered once daily. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered twice daily.

In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered once daily. In some embodiments, at least one compound chosen from compounds of compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered twice daily.

In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing are administered once daily. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts of any of the foregoing are administered twice daily.

In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing are administered once daily. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing are administered twice daily.

In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, and at least one compound chosen from lumacaftor and deuterated derivatives and pharmaceutically acceptable salts thereof, are administered once daily. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, and at least one compound chosen from lumacaftor and deuterated derivatives and pharmaceutically acceptable salts thereof, are administered twice daily.

In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof, are administered once daily and at least one compound chosen from ivacaftor and deuterated derivatives and pharmaceutically acceptable salts thereof, are administered twice daily. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, and at least one compound chosen from lumacaftor and deuterated derivatives and pharmaceutically acceptable salts thereof, are administered once daily and at least one compound chosen from ivacaftor and deuterated derivatives and pharmaceutically acceptable salts thereof, are administered twice daily.

In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof, are administered once daily and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof, are administered once or twice daily. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, and at least one compound chosen from lumacaftor and deuterated derivatives and pharmaceutically acceptable salts thereof, are administered once daily and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof, are administered once or twice daily.

Compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, at least one compound selected fromtezacaftor, lumacaftor, and deuterated derivatives and pharmaceutically acceptable salts of tezacaftor and lumacavtor, and/or at least one compound selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing can be administered in a single pharmaceutical composition or separate pharmaceutical compositions. Such pharmaceutical compositions can be administered once daily or multiple times daily, such as twice daily. As used herein, the phrase that a given amount of API (such as a CFTR modulator, e.g., tezacaftor, lumacaftor, ivacaftor, deutivacaftor, and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, or a deuterated derivative or pharmaceutically acceptable salt thereof) is administered once or twice daily or per day means that said given amount is administered per dosing once or twice daily.

In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; and at least one compound chosen from ivacaftor and deuterated derivatives and pharmaceutically acceptable salts thereof is administered in a third pharmaceutical composition. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; at least one compound chosen from deutivacaftor and deuterated derivatives and pharmaceutically acceptable salts thereof is administered in a third pharmaceutical composition. In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in a first pharmaceutical composition; at least one compound chosen from tezacaftor, lumacaftor, and deuterated derivatives and pharmaceutically acceptable salts thereof is administered in a second pharmaceutical composition; and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof is administered in a third pharmaceutical composition.

In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in a first pharmaceutical composition; at least one compound chosen from ivacftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing is administered in a second pharmaceutical composition; at least one compound chosen from lumacaftor and deuterated derivatives and pharmaceutically acceptable salts thereof is administered in a third pharmaceutical composition.

In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, is administered in a first pharmaceutical composition; and at least one compound chosen from tezacaftor, lumacaftor and deuterated derivatives and pharmaceutically acceptable salts thereof and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing are administered in a second pharmaceutical composition. In some embodiments, the second pharmaceutical composition comprises a half of a daily dose of ivacaftor, and the other half dose of ivacaftor is administered in a third pharmaceutical composition.

In some embodiments, at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing; at least one compound chosen from tezacaftor and deuterated derivatives and pharmaceutically acceptable salts thereof and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing are administered in a first pharmaceutical composition. In some embodiments, the first pharmaceutical composition is administered to the patient twice daily. In some embodiments the first pharmaceutical composition is administered once daily. In some embodiments the first pharmaceutical composition is administered once daily and a second composition comprising only ivacaftor is administered once daily.

Any suitable pharmaceutical compositions can be used for compounds of Formula I, compounds of any one of Formulae Ia, IIa, IIb, III, IV, V, VIa, and VIb, Compounds 1 to 369, tezacaftor, ivacaftor, deutivacaftor, lumacaftor and tautomers thereof, deuterated derivatives of those compounds and tautomers, and pharmaceutically acceptable salts of any of the foregoing. Some exemplary pharmaceutical compositions for tezacaftor and its pharmaceutically acceptable salts can be found in WO 2011/119984 and WO 2014/014841, incorporated herein by reference. Some exemplary pharmaceutical compositions for ivacaftor and its pharmaceutically acceptable salts can be found in WO 2007/134279, WO 2010/019239, WO 2011/019413, WO 2012/027731, and WO 2013/130669, and some exemplary pharmaceutical compositions for deutivacaftor and its pharmaceutically acceptable salts can be found in U.S. Pat. Nos. 8,865,902, 9,181,192, 9,512,079, WO 2017/053455, and WO 2018/080591, all of which are incorporated herein by reference. Some exemplary pharmaceutical compositions for lumacaftor and its pharmaceutically acceptable salts can be found in WO 2010/037066, WO 2011/127421, and WO 2014/071122, incorporated herein by reference.

### Pharmaceutical Compositions

Another aspect of the disclosure provides a pharmaceutical composition comprising at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, and at least one pharmaceutically acceptable carrier.

In some embodiments, the disclosure provides pharmaceutical compositions comprising at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, and at least two additional active pharmaceutical ingredients, one of which is a CFTR corrector and one of which is a CFTR potentiator.

In some embodiments, the disclosure provides a pharmaceutical composition comprising (a) at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, (b) at least one compound chosen from tezacaftor, lumacaftor, and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, (b) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, and (c) at least one pharmaceutically acceptable carrier.

In some embodiments, the disclosure provides a pharmaceutical composition comprising (a) at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from ivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of any one of Formulae Ia, IIa, IIb, III, IV, V, VIa, and VIb, Compounds 1-369, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from deutivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of any one of Formulae Ia, IIa, IIb, III, IV, V, VIa, and VIb, Compounds 1 to 369, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of any one of Formulae Ia, IIa, IIb, III, IV, V, VIa, and VIb, Compounds 1-369, tautomers thereof, deuterated derivatives of those compounds and tautomers, and pharmaceutically acceptable salts of any of the foregoing, (b) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, (c) at least one compound chosen from lumacaftor and pharmaceutically acceptable salts thereof, and (d) at least one pharmaceutically acceptable carrier.

Any pharmaceutical composition disclosed herein may comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, lubricants.

The pharmaceutical compositions described herein are useful for treating cystic fibrosis and other CFTR mediated diseases. The compounds and compositions disclosed herein may be used in the manufacture of medicaments to treat cystic fibrosis and other CFTR mediated diseases.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### EXEMPLARY EMBODIMENTS

The following is a non-limiting list of exemplary embodiments:
1. A compound of Formula I:

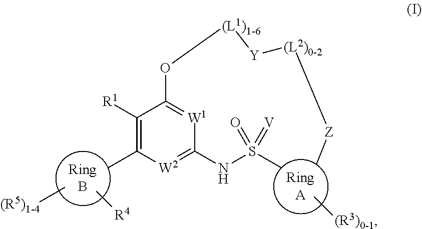

a tautomer thereof, a deuterated derivative of the compound or tautomer, or a pharmaceutically acceptable salt of any of the foregoing, wherein:
   Ring A is selected from:
      C₆-C₁₀ aryl,
      C₃-C₁₀ cycloalkyl,
      3- to 10-membered heterocyclyl, and
      5- to 10-membered heteroaryl;
   Ring B is selected from:
      C₆-C₁₀ aryl,
      C₃-C₁₀ cycloalkyl,
      3- to 10-membered heterocyclyl, and
      5- to 10-membered heteroaryl;
   V is selected from O and NH;
   W¹ is selected from N and CH;
   W² is selected from N and CH; provided that at least one of W¹ and W² is N;
   Y is selected from O, NR^{YN}, and C(R^{YC})₂.
   Z is selected from O, NR^{ZN}, and C(R^{ZC})₂, provided that when L² is absent, either Y is C(R^{YC})₂ or Z is C(R^{ZC})₂;
   each L¹ is independently selected from C(R^{L1})₂ and

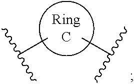

each L² is independently selected from C(R^{L2})₂;
Ring C is selected from C₆-C₁₀ aryl optionally substituted with 1-3 groups independently selected from:
   halogen,
   C₁-C₆ alkyl, and
   N(R^{N})₂;
R¹ is selected from:
   halogen,
   cyano,
   C₁-C₆ alkyl optionally substituted with 1-3 groups independently selected from hydroxyl, oxo, and N(R^{N})₂,
   C₁-C₆ alkoxy,
   C₁-C₆ fluoroalkyl,
   C₆-C₁₀ aryl optionally substituted with 1-3 groups independently selected from C₁-C₆ alkoxy,
   3- to 10-membered heterocyclyl optionally substituted with 1-3 groups independently selected from R^{N}, and
   5- to 10-membered heteroaryl optionally substituted with 1-3 groups independently selected from C₁-C₆ alkyl;
each R³ is independently selected from:
   halogen,
   C₁-C₆ alkyl,
   C₁-C₆ alkoxy,
   C₃-C₁₀ cycloalkyl,
   C₆-C₁₀ aryl optionally substituted with 1-3 groups independently selected from C₁-C₆ alkyl, and
   3- to 10-membered heterocyclyl;
R⁴ is selected from hydrogen and C₁-C₆ alkyl;
each R⁵ is independently selected from:
   hydrogen,
   halogen,
   hydroxyl,
   N(R^{N})₂,
   SO-Me,
   CH═C(R^{LC})₂, wherein both R^{LC} are taken together to form a C₃-C₁₀ cycloalkyl,
   C₁-C₆ alkyl optionally substituted with 1-3 groups independently selected from:
   hydroxyl,
   C₁-C₆ alkoxy optionally substituted with 1-3 groups independently selected from C₁-C₆ alkoxy and C₆-C₁₀ aryl,
   C₃-C₁₀ cycloalkyl,
   —(O)₀₋₁—(C₆-C₁₀ aryl) optionally substituted with 1-3 groups independently selected from C₁-C₆ alkyl and C₁-C₆ alkoxy,
   3- to 10-membered heterocyclyl, and
   N(R^{N})₂,
   C₁-C₆ alkoxy optionally substituted with 1-3 groups independently selected from:
   halogen,
   C₆-C₁₀ aryl, and
   C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl,
   C₆-C₁₀ aryl, and
   3- to 10-membered heterocyclyl;
each R^{YN} and R^{ZN} is independently selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
      hydroxyl,
      C₁-C₆ alkyl optionally substituted with 1-3 groups independently selected from hydroxyl, oxo, C₁-C₆ alkoxy, C₆-C₁₀ aryl, and N(R^{N})₂,
      C₁-C₆ fluoroalkyl,
      C₁-C₆ alkoxy, and
      COOH,
      N(R^{N})₂,
      C₆-C₁₀ aryl, and
      3- to 10-membered heterocyclyl optionally substituted with 1-3 groups independently selected from oxo and C₁-C₆ alkyl,
      C₆-C₁₀ aryl optionally substituted with 1-3 groups independently selected from:
      halogen,
      hydroxyl,
      cyano,
      SiMe₃,
      SO₂Me,
      SF₅,
      N(R^{N})₂,
      P(O)Me₂,
      —(O)₀₋₁—(C₃-C₁₀ cycloalkyl) optionally substituted with 1-3 groups independently selected from C₁-C₆ fluoroalkyl,
      C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
      C₁-C₆ alkoxy optionally substituted with 1-3 groups independently selected from hydroxyl, oxo, N(R^{N})₂, and C₆-C₁₀ aryl,
      C₁-C₆ fluoroalkyl,
      3- to 10-membered heterocyclyl optionally substituted with 1-3 groups independently selected from C₁-C₆ alkyl,
      —(O)₀₋₁—(C₆-C₁₀ aryl), and
      —(O)₀₋₁-(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
      hydroxyl,
      oxo,
      N(R^{N})₂,
      C₁-C₆ alkyl (optionally substituted with 1-3 groups independently selected from oxo and C₁-C₆ alkoxy),
      C₁-C₆ alkoxy,
      C₁-C₆ fluoroalkyl,
      C₆-C₁₀ aryl optionally substituted with 1-3 groups independently selected from halogen, and
      5- to 10-membered heteroaryl, and
      5- to 10-membered heteroaryl optionally substituted with 1-3 groups independently selected from:
      hydroxyl,
      cyano,
      oxo,
      halogen,
      B(OH)₂,
      N(R^{N})₂,
      C₁-C₆ alkyl optionally substituted with 1-3 groups independently selected from hydroxyl, oxo, C₁-C₆ alkoxy (optionally substituted with 1-3-SiMe₃), and N(R^{N})₂,
      C₁-C₆ alkoxy optionally substituted with 1-3 groups independently selected from hydroxyl, oxo, C₁-C₆ alkoxy, N(R^{N})₂, and C₃-C₁₀ cycloalkyl,
      C₁-C₆ fluoroalkyl,
      —(O)₀₋₁—(C₃-C₁₀ cycloalkyl) optionally substituted with 1-3 groups independently selected from halogen and C₁-C₆ alkyl,
      —(O)₀₋₁—(C₆-C₁₀ aryl),
      —(O)₀₋₁-(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl) and
      5- to 10-membered heteroaryl optionally substituted with 1-4 groups independently selected from C₁-C₆ alkyl and C₃-C₁₀ cycloalkyl,
   C₁-C₆ fluoroalkyl,
   C₃-C₁₀ cycloalkyl optionally substituted with 1-3 groups independently selected from:
      hydroxyl,
      oxo,
      halogen,
      cyano,
      N(R^{N})₂,
      C₁-C₆ alkyl optionally substituted with 1-3 groups independently selected from:
      hydroxyl,
      oxo,
      N(R^{N})₂,
      C₁-C₆ alkoxy, and
      C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-member heterocyclyl optionally substituted with 1-3 groups independently selected from C₁-C₆ alkyl, and
      5- to 10-membered heteroaryl optionally substituted with 1-3 groups independently selected from:
      hydroxyl,
      cyano,
      oxo,
      halogen,
      N(R^{N})₂,
      C₁-C₆ alkyl optionally substituted with 1-3 groups independently selected from hydroxyl, oxo, C₁-C₆ alkoxy, and N(R^{N})₂,
      C₁-C₆ alkoxy optionally substituted with 1-3 groups independently selected from hydroxyl, C₁-C₆ alkoxy, N(R^{N})₂, and C₃-C₁₀ cycloalkyl,
      C₁-C₆ fluoroalkyl,
      —(O)₀₋₁—(C₃-C₁₀ cycloalkyl) optionally substituted with 1-3 groups independently selected from C₁-C₆ alkyl,
      C₆-C₁₀ aryl, and
      3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
      oxo,
      hydroxyl,
      N(R^{N})₂,
      C₁-C₆ alkoxy optionally substituted with 1-3 groups independently selected from halogen and C₆-C₁₀ aryl, and
      —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl,
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
   R^{F};
each R^{YC} and R^{ZC} is independently selected from:
   hydrogen,
   C₁-C₆ alkyl optionally substituted with 1-3 groups independently selected from C₆-C₁₀ aryl (optionally substituted with 1-3 groups independently selected from C₁-C₆ alkyl),
   C₆-C₁₀ aryl optionally substituted with 1-3 groups independently selected from C₁-C₆ alkyl, and
   R^{F};
or two R^{YC} are taken together to form an oxo group;
or two R^{ZC} are taken together to form an oxo group;
each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl optionally substituted with 1-3 groups independently selected from C₁-C₆ alkyl (optionally substituted with 1-3 groups independently selected from hydroxyl and oxo),
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from:
      hydroxyl,
      oxo,
      cyano,
      SiMe₃,
      N(R^{N})₂, and
      C₃-C₁₀ cycloalkyl optionally substituted with 1-3 groups independently selected from C₁-C₆ fluoroalkyl,
      C₁-C₆ alkoxy optionally substituted with 1-3 groups independently selected from:
      C₃-C₁₀ cycloalkyl optionally substituted with 1-3 groups independently selected from C₁-C₆ fluoroalkyl, and
      C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl,
      3- to 10-membered heterocyclyl optionally substituted with 1-3 groups independently selected from C₁-C₆ alkyl, and
      5- to 10-membered heteroaryl,
   3- to 10-membered heterocyclyl optionally substituted with 1-3 groups independently selected from:
      C₁-C₆ alkyl optionally substituted with 1-3 groups independently selected from:
      oxo, and
      C₁-C₆ alkoxy,
   5- to 10-membered heteroaryl optionally substituted with 1-3 groups independently selected from:
      C₁-C₆ alkyl optionally substituted with 1-3 groups independently selected from:
      C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl, and
   R^{F};
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
   hydrogen,
   C₁-C₈ alkyl optionally substituted with 1-3 groups independently selected from:
      oxo,
      halogen,
      hydroxyl,
      NH₂,
      NHMe,
      NMe₂,
      C₁-C₆ alkoxy optionally substituted with 1-3 groups independently selected from C₆-C₁₀ aryl,
      —(O)₀₋₁—(C₃-C₁₀ cycloalkyl),
      C₆-C₁₀ aryl optionally substituted with 1-3 groups independently selected from halogen and C₁-C₆ alkyl, and
      3- to 14-membered heterocyclyl optionally substituted with 1-4 groups independently selected from oxo and C₁-C₆ alkyl, and
      5- to 14-membered heteroaryl optionally substituted with 1-4 groups independently selected from oxo and C₁-C₆ alkyl,
   C₃-C₁₀ cycloalkyl optionally substituted with 1-3 groups independently selected from:
      hydroxyl,
      NH₂, and
      NHMe, and
      C₁-C₆ alkyl optionally substituted with 1-3 groups independently selected from hydroxyl, and
   C₆-C₁₀ aryl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from:
   hydroxyl,
   oxo,
   cyano,
   C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
   C₁-C₆ alkoxy, and
   C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
   C₃-C₁₀ cycloalkyl optionally substituted with 1-3 groups independently selected from C₁-C₆ alkyl,
   C₆-C₁₀ aryl optionally substituted with 1-3 groups independently selected from:
      halogen,
      C₁-C₆ alkyl,
      N(R^{N})₂, and
      3- to 10-membered heterocyclyl optionally substituted with 1-3 groups independently selected from hydroxyl,
   3- to 11-membered heterocyclyl optionally substituted with 1-3 groups independently selected from:
      oxo,
      N(R^{N})₂,
      C₁-C₉ alkyl optionally substituted with 1-4 groups independently selected from:
      oxo,
      halogen,
      hydroxyl,
      N(R^{N})₂,
      -SO₂-(C₁-C₆ alkyl),
      C₁-C₆ alkoxy optionally substituted with 1-3 groups independently selected from halogen, C₆-C₁₀ aryl,
      C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
      —(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
      3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
      —O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
      5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₃-C₁₂ cycloalkyl optionally substituted with 1-4 groups independently selected from halogen, C₁-C₆ alkyl, and C₁-C₆ fluoroalkyl,
      C₆-C₁₀ aryl,
      3- to 10-membered heterocyclyl, and
      5- to 10-membered heteroaryl optionally substituted with 1-3 groups independently selected from C₁-C₆ alkoxy and C₁-C₆ fluoroalkyl, and
5- to 12-membered heteroaryl optionally substituted with 1-3 groups independently selected from C₁-C₆ alkyl and C₁-C₆ fluoroalkyl.
2. The compound, tautomer, deuterated derivative, or pharmaceutically acceptable salt according to embodiment 1, wherein Ring A is selected from selected from C₆-C₁₀ aryl.
3. The compound, tautomer, deuterated derivative, or pharmaceutically acceptable salt according to embodiment 1 or 2, wherein Ring A is phenyl.
4. The compound, tautomer, deuterated derivative, or pharmaceutically acceptable salt according to any one of embodiments 1 to 3, wherein Ring B is selected from C₆-C₁₀ aryl and 5- to 10-membered heteroaryl.
5. The compound, tautomer, deuterated derivative, or pharmaceutically acceptable salt according to any one of embodiments 1 to 4, wherein Ring B is selected from phenyl and pyridinyl.
6. The compound, tautomer, deuterated derivative, or pharmaceutically acceptable salt according to any one of embodiments 1 to 5, wherein Ring B is phenyl.
7. The compound, tautomer, deuterated derivative, or pharmaceutically acceptable salt according to any one of embodiments 1 to 6, wherein V is O.
8. The compound, tautomer, deuterated derivative, or pharmaceutically acceptable salt according to any one of embodiments 1 to 7, wherein W¹ is N and W² is N.
9. The compound, tautomer, deuterated derivative, or pharmaceutically acceptable salt according to any one of embodiments 1 to 8, wherein Y is selected from NR^{YN} and C(R^{YC})₂.
10. The compound, tautomer, deuterated derivative, or pharmaceutically acceptable salt according to any one of embodiments 1 to 9, wherein Z is selected from NR^{ZN}, and C(R^{ZC})₂, provided that when L² is absent, either Y is C(R^{YC})₂ or Z is C(R^{ZC})₂
11. The compound, tautomer, deuterated derivative, or pharmaceutically acceptable salt according to any one of embodiments 1 to 10, wherein Ring C is a phenyl optionally substituted with 1-3 groups independently selected from:
   halogen,
   C₁-C₆ alkyl, and
   N(R^{N})₂.
12. The compound, tautomer, deuterated derivative, or pharmaceutically acceptable salt according to any one of embodiments 1 to 11, wherein R³ is absent.
13. The compound, tautomer, deuterated derivative, or pharmaceutically acceptable salt according to any one of embodiments 1 to 12, wherein R⁴ is selected from hydrogen and methyl.
14. The compound, tautomer, deuterated derivative, or pharmaceutically acceptable salt according to any one of embodiments 1 to 13, wherein R⁴ is methyl.
15. The compound, tautomer, deuterated derivative, or pharmaceutically acceptable salt according to any one of embodiments 1 to 14, wherein each R⁵ is independently selected from:
   hydrogen,
   halogen,
   SO-Me,
   C₁-C₆ alkyl optionally substituted with 1-3 groups independently selected from:
      C₁-C₆ alkoxy optionally substituted with 1-3 groups independently selected from C₁-C₆ alkoxy,
      C₃-C₁₀ cycloalkyl,
      —(O)₀₋₁—(C₆-C₁₀ aryl) optionally substituted with 1-3 groups independently selected from C₁-C₆ alkyl,
      3- to 10-membered heterocyclyl, and
      N(R^{N})₂,
   C₁-C₆ alkoxy,
   C₁-C₆ fluoroalkyl,
   C₃-C₁₀ cycloalkyl, and
   3- to 10-membered heterocyclyl.
16. The compound, tautomer, deuterated derivative, or pharmaceutically acceptable salt according to any one of embodiments 1 to 15, wherein each R^{YN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
   C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
R^{F}.
17. The compound, tautomer, deuterated derivative, or pharmaceutically acceptable salt according to any one of embodiments 1 to 16, wherein each R^{ZN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl),
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
18. The compound, tautomer, deuterated derivative, or pharmaceutically acceptable salt according to any one of embodiments 1 to 17, wherein each C(R^{YC})₂ is C═O.
19. The compound, tautomer, deuterated derivative, or pharmaceutically acceptable salt according to any one of embodiments 1 to 18, wherein each C(R^{ZC})₂ is C═O.
20. The compound, tautomer, deuterated derivative, or pharmaceutically acceptable salt according to any one of embodiments 1 to 19, wherein each R^{L1} is independently selected from:
   hydrogen,
   C₁-C₉ alkyl optionally substituted with 1-3 groups independently selected from:
      halogen,
      hydroxyl,
      C₃-C₁₀ cycloalkyl optionally substituted with 1-3 groups independently selected from C₁-C₆ fluoroalkyl,
   C₆-C₁₀ aryl optionally substituted with 1-4 groups independently selected from C₁-C₇ alkyl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
   R^{F}.
21. The compound, tautomer, deuterated derivative, or pharmaceutically acceptable salt according to any one of embodiments 1 to 20, wherein L² is absent.
22. The compound, tautomer, deuterated derivative, or pharmaceutically acceptable salt according to any one of embodiments 1 to 21, wherein each R^{N} is independently selected from C₁-C₈ alkyl optionally substituted with 1-3 groups independently selected from oxo and C₁-C₆ alkoxy.
23. The compound, tautomer, deuterated derivative, or pharmaceutically acceptable salt according to any one of embodiments 1 to 22, wherein when R^{F} is present, two R^{F} taken together with the atoms to which they are bonded form a group selected from:
   C₆-C₁₀ aryl optionally substituted with 1-3 groups independently selected from halogen and C₁-C₆ alkyl, and
   3- to 11-membered heterocyclyl optionally substituted with 1-3 groups independently selected from:
      C₁-C₉ alkyl optionally substituted with 1-4 groups independently selected from:
         —(O)₀₋₁—(C₃-C₁₀ cycloalkyl) optionally substituted with 1-4 groups independently selected from C₁-C₆ alkyl and C₁-C₆ fluoroalkyl,
      C₃-C₁₂ cycloalkyl, and
      C₆-C₁₀ aryl.
24. A compound of Formula Ia:

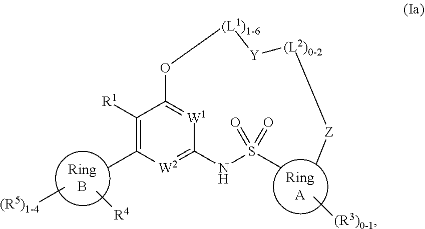

a tautomer thereof, a deuterated derivative of the compound or tautomer, or a pharmaceutically acceptable salt of any of the foregoing, wherein Ring A, Ring B, W¹, W², Y, Z, L¹, L², R¹, R³, R⁴, and R⁵ are defined as according to embodiment 1.
25. The compound, tautomer, deuterated derivative, or pharmaceutically acceptable salt according to embodiment 24, wherein Ring A is selected from selected from C₆-C₁₀ aryl.
26. The compound, tautomer, deuterated derivative, or pharmaceutically acceptable salt according to embodiment 24 or 25, wherein Ring A is phenyl.
27. The compound, tautomer, deuterated derivative, or pharmaceutically acceptable salt according to any one of embodiments 24 to 26, wherein Ring B is selected from C₆-C₁₀ aryl and 5- to 10-membered heteroaryl.
28. The compound, tautomer, deuterated derivative, or pharmaceutically acceptable salt according to any one of embodiments 24 to 27, wherein Ring B is selected from phenyl and pyridinyl.
29. The compound, tautomer, deuterated derivative, or pharmaceutically acceptable salt according to any one of embodiments 24 to 28, wherein Ring B is phenyl.
30. The compound, tautomer, deuterated derivative, or pharmaceutically acceptable salt according to any one of embodiments 24 to 29, wherein W¹ is N and W² is N.
31. The compound, tautomer, deuterated derivative, or pharmaceutically acceptable salt according to any one of embodiments 24 to 30, wherein Y is selected from NR^{YN} and C(R^{YC})₂.
32. The compound, tautomer, deuterated derivative, or pharmaceutically acceptable salt according to any one of embodiments 24 to 31, wherein Z is selected from NR^{ZN}, and C(R^{ZC})₂, provided that when L² is absent, either Y is C(R^{YC})₂ or Z is C(R^{ZC})₂
33. The compound, tautomer, deuterated derivative, or pharmaceutically acceptable salt according to any one of embodiments 24 to 32, wherein Ring C is a phenyl optionally substituted with 1-3 groups independently selected from:
   halogen,
   C₁-C₆ alkyl, and
   N(R^{N})₂.
34. The compound, tautomer, deuterated derivative, or pharmaceutically acceptable salt according to any one of embodiments 24 to 33, wherein R³ is absent.
35. The compound, tautomer, deuterated derivative, or pharmaceutically acceptable salt according to any one of embodiments 24 to 34, wherein R⁴ is selected from hydrogen and methyl.
36. The compound, tautomer, deuterated derivative, or pharmaceutically acceptable salt according to any one of embodiments 24 to 35, wherein R⁴ is methyl.
37. The compound, tautomer, deuterated derivative, or pharmaceutically acceptable salt according to any one of embodiments 24 to 36, wherein each R⁵ is independently selected from:
   hydrogen,
   halogen,
   SO-Me,
   C₁-C₆ alkyl optionally substituted with 1-3 groups independently selected from:
      C₁-C₆ alkoxy optionally substituted with 1-3 groups independently selected from C₁-C₆ alkoxy,
      C₃-C₁₀ cycloalkyl,
      —(O)₀₋₁—(C₆-C₁₀ aryl) optionally substituted with 1-3 groups independently selected from C₁-C₆ alkyl,
      3- to 10-membered heterocyclyl, and
      N(R^{N})₂,
   C₁-C₆ alkoxy,
   C₁-C₆ fluoroalkyl,
   C₃-C₁₀ cycloalkyl, and
   3- to 10-membered heterocyclyl.
38. The compound, tautomer, deuterated derivative, or pharmaceutically acceptable salt according to any one of embodiments 24 to 37, wherein each R^{YN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
3- to 10-membered heterocyclyl optionally substituted with 1-3 groups independently selected from C₁-C₆ alkyl (optionally substituted with 1-3 groups independently selected from oxo and C₁-C₆ alkoxy),
5- to 10-membered heteroaryl optionally substituted with 1-3 groups independently selected from halogen and C₁-C₆ alkyl, and
R^{F}.
39. The compound, tautomer, deuterated derivative, or pharmaceutically acceptable salt according to any one of embodiments 24 to 38, wherein each R^{ZN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
40. The compound, tautomer, deuterated derivative, or pharmaceutically acceptable salt according to any one of embodiments 24 to 39, wherein each C(R^{YC})₂ is C═O.
41. The compound, tautomer, deuterated derivative, or pharmaceutically acceptable salt according to any one of embodiments 24 to 40, wherein each C(R^{ZC})₂ is C═O.
42. The compound, tautomer, deuterated derivative, or pharmaceutically acceptable salt according to any one of embodiments 24 to 41, wherein each R^{L1} is independently selected from:
   hydrogen,
   C₁-C₉ alkyl optionally substituted with 1-3 groups independently selected from:
      halogen,
      hydroxyl, and
      C₃-C₁₀ cycloalkyl optionally substituted with 1-3 groups independently selected from C₁-C₆ fluoroalkyl,
   C₆-C₁₀ aryl optionally substituted with 1-4 groups independently selected from C₁-C₇ alkyl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
   R^{F}.
43. The compound, tautomer, deuterated derivative, or pharmaceutically acceptable salt according to any one of embodiments 24 to 42, wherein L² is absent.
44. The compound, tautomer, deuterated derivative, or pharmaceutically acceptable salt according to any one of embodiments 24 to 43, wherein each R^{N} is independently selected from C₁-C₈ alkyl optionally substituted with 1-3 groups independently selected from oxo and C₁-C₆ alkoxy.
45. The compound, tautomer, deuterated derivative, or pharmaceutically acceptable salt according to any one of embodiments 24 to 44, wherein when R^{F} is present, two R^{F} taken together with the atoms to which they are bonded form a group selected from:
   C₆-C₁₀ aryl optionally substituted with 1-3 groups independently selected from halogen and C₁-C₆ alkyl, and
   3- to 11-membered heterocyclyl optionally substituted with 1-3 groups independently selected from:
      C₁-C₉ alkyl optionally substituted with 1-4 groups independently selected from:
         —(O)₀₋₁—(C₃-C₁₀ cycloalkyl) optionally substituted with 1-4 groups independently selected from C₁-C₆ alkyl and C₁-C₆ fluoroalkyl,
      C₃-C₁₂ cycloalkyl, and
      C₆-C₁₀ aryl.
46. A compound of Formula IIa:

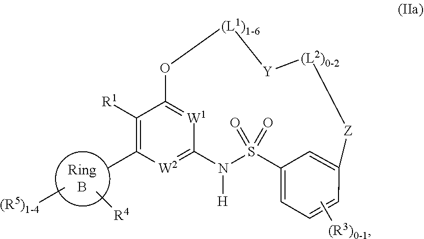

a tautomer thereof, a deuterated derivative of the compound or tautomer, or a pharmaceutically acceptable salt of any of the foregoing, wherein Ring B, W¹, W², X, Y, Z, L¹, L², R¹, R³, R⁴, and R⁵ are defined as according to embodiment 1.
47. The compound, tautomer, deuterated derivative, or pharmaceutically acceptable salt according to embodiment 46, wherein Ring B is selected from C₆-C₁₀ aryl and 5- to 10-membered heteroaryl.
48. The compound, tautomer, deuterated derivative, or pharmaceutically acceptable salt according to embodiment 46 or 47, wherein Ring B is selected from phenyl and pyridinyl.
49. The compound, tautomer, deuterated derivative, or pharmaceutically acceptable salt according to any one of embodiments 46 to 48, wherein Ring B is phenyl.
50. The compound, tautomer, deuterated derivative, or pharmaceutically acceptable salt according to any one of embodiments 46 to 49, wherein W¹ is N and W² is N.
51. The compound, tautomer, deuterated derivative, or pharmaceutically acceptable salt according to any one of embodiments 46 to 50, wherein Y is selected from NR^{YN} and C(R^{YC})₂.
52. The compound, tautomer, deuterated derivative, or pharmaceutically acceptable salt according to any one of embodiments 46 to 51, wherein Z is selected from NR^{ZN}, and C(R^{ZC})₂, provided that when L² is absent, either Y is C(R^{YC})₂ or Z is C(R^{ZC})₂
53. The compound, tautomer, deuterated derivative, or pharmaceutically acceptable salt according to any one of embodiments 46 to 52, wherein Ring C is a phenyl optionally substituted with 1-3 groups independently selected from:
   halogen,
   C₁-C₆ alkyl, and
   N(R^{N})₂.
54. The compound, tautomer, deuterated derivative, or pharmaceutically acceptable salt according to any one of embodiments 46 to 53, wherein R³ is absent.
55. The compound, tautomer, deuterated derivative, or pharmaceutically acceptable salt according to any one of embodiments 46 to 54, wherein R⁴ is selected from hydrogen and methyl.
56. The compound, tautomer, deuterated derivative, or pharmaceutically acceptable salt according to any one of embodiments 46 to 55, wherein R⁴ is methyl.
57. The compound, tautomer, deuterated derivative, or pharmaceutically acceptable salt according to any one of embodiments 46 to 56, wherein each R⁵ is independently selected from:
   hydrogen,
   halogen,
   SO-Me,
   C₁-C₆ alkyl optionally substituted with 1-3 groups independently selected from:
      C₁-C₆ alkoxy optionally substituted with 1-3 groups independently selected from C₁-C₆ alkoxy,
      C₃-C₁₀ cycloalkyl,
      —(O)₀₋₁—(C₆-C₁₀ aryl) optionally substituted with 1-3 groups independently selected from C₁-C₆ alkyl,
      3- to 10-membered heterocyclyl, and
      N(R^{N})₂,
   C₁-C₆ alkoxy,
   C₁-C₆ fluoroalkyl,
   C₃-C₁₀ cycloalkyl, and
   3- to 10-membered heterocyclyl.
58. The compound, tautomer, deuterated derivative, or pharmaceutically acceptable salt according to any one of embodiments 46 to 57, wherein each R^{YN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         (O)₀₋₁—(C₃-C₁₀ cycloalkyl), and
         (O)₀₋₁-(3- to 10-membered heterocyclyl) optionally substituted with 1-4 groups independently selected from C₁-C₆ alkyl,
      C₃-C₁₀ cycloalkyl optionally substituted with 1-3 groups independently selected from:
         hydroxyl,
         C₁-C₆ alkyl optionally substituted with 1-3 groups independently selected from hydroxyl, and
         C₁-C₆ alkoxy,
      3- to 10-membered heterocyclyl optionally substituted with 1-3 groups independently selected from C₁-C₆ alkyl (optionally substituted with 1-3 groups independently selected from oxo and C₁-C₆ alkoxy),
      5- to 10-membered heteroaryl optionally substituted with 1-3 groups independently selected from halogen and C₁-C₆ alkyl, and
      R^{F}.
59. The compound, tautomer, deuterated derivative, or pharmaceutically acceptable salt according to any one of embodiments 46 to 58, wherein each R^{ZN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
60. The compound, tautomer, deuterated derivative, or pharmaceutically acceptable salt according to any one of embodiments 46 to 59, wherein each C(R^{YC})₂ is C═O.
61. The compound, tautomer, deuterated derivative, or pharmaceutically acceptable salt according to any one of embodiments 46 to 60, wherein each C(R^{ZC})₂ is C═O.
62. The compound, tautomer, deuterated derivative, or pharmaceutically acceptable salt according to any one of embodiments 46 to 61, wherein each R^{L1} is independently selected from:
   hydrogen,
   C₁-C₉ alkyl optionally substituted with 1-3 groups independently selected from:
      halogen,
      hydroxyl, and
      C₃-C₁₀ cycloalkyl optionally substituted with 1-3 groups independently selected from C₁-C₆ fluoroalkyl,
   C₆-C₁₀ aryl optionally substituted with 1-4 groups independently selected from C₁-C₇ alkyl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
   R^{F}.
63. The compound, tautomer, deuterated derivative, or pharmaceutically acceptable salt according to any one of embodiments 46 to 62, wherein L² is absent.
64. The compound, tautomer, deuterated derivative, or pharmaceutically acceptable salt according to any one of embodiments 46 to 63, wherein each R^{N} is independently selected from C₁-C₈ alkyl optionally substituted with 1-3 groups independently selected from oxo and C₁-C₆ alkoxy.
65. The compound, tautomer, deuterated derivative, or pharmaceutically acceptable salt according to any one of embodiments 46 to 64, wherein when R^{F} is present, two R^{F} taken together with the atoms to which they are bonded form a group selected from:
   C₆-C₁₀ aryl optionally substituted with 1-3 groups independently selected from halogen and C₁-C₆ alkyl, and
   3- to 11-membered heterocyclyl optionally substituted with 1-3 groups independently selected from:
      C₁-C₉ alkyl optionally substituted with 1-4 groups independently selected from:
         —(O)₀₋₁—(C₃-C₁₀ cycloalkyl) optionally substituted with 1-4 groups independently selected from C₁-C₆ alkyl and C₁-C₆ fluoroalkyl,
      C₃-C₁₂ cycloalkyl, and
      C₆-C₁₀ aryl.
66. A compound of Formula IIb:

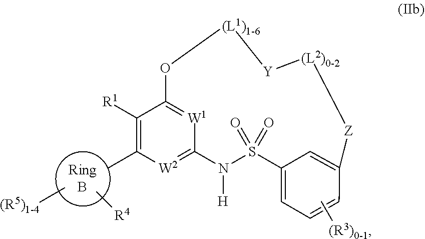

a tautomer thereof, a deuterated derivative of the compound or tautomer, or a pharmaceutically acceptable salt of any of the foregoing, wherein Ring A, W¹, W², Y, Z, L¹, L², R¹, R³, R⁴, and R⁵ are defined as according to embodiment 1.
67. The compound, tautomer, deuterated derivative, or pharmaceutically acceptable salt according to embodiment 66, wherein Ring A is selected from selected from C₆-C₁₀ aryl.
68. The compound, tautomer, deuterated derivative, or pharmaceutically acceptable salt according to embodiment 66 or 67, wherein Ring A is phenyl.
69. The compound, tautomer, deuterated derivative, or pharmaceutically acceptable salt according to any one of embodiments 66 to 68, wherein W¹ is N and W² is N.
70. The compound, tautomer, deuterated derivative, or pharmaceutically acceptable salt according to any one of embodiments 66 to 69, wherein Y is selected from NR^{Y}N and C(R^{YC})₂.
71. The compound, tautomer, deuterated derivative, or pharmaceutically acceptable salt according to any one of embodiments 66 to 70, wherein Z is selected from NR^{ZN}, and C(R^{ZC})₂, provided that when L² is absent, either Y is C(R^{YC})₂ or Z is C(R^{ZC})₂
72. The compound, tautomer, deuterated derivative, or pharmaceutically acceptable salt according to any one of embodiments 66 to 71, wherein Ring C is a phenyl optionally substituted with 1-3 groups independently selected from:
   halogen,
   C₁-C₆ alkyl, and
   N(R^{N})₂.
73. The compound, tautomer, deuterated derivative, or pharmaceutically acceptable salt according to any one of embodiments 66 to 72, wherein R³ is absent.
74. The compound, tautomer, deuterated derivative, or pharmaceutically acceptable salt according to any one of embodiments 66 to 73, wherein R⁴ is selected from hydrogen and methyl.
75. The compound, tautomer, deuterated derivative, or pharmaceutically acceptable salt according to any one of embodiments 66 to 74, wherein R⁴ is methyl.
76. The compound, tautomer, deuterated derivative, or pharmaceutically acceptable salt according to any one of embodiments 66 to 75, wherein each R⁵ is independently selected from:
   hydrogen,
   halogen,
   SO-Me,
   C₁-C₆ alkyl optionally substituted with 1-3 groups independently selected from:
      C₁-C₆ alkoxy optionally substituted with 1-3 groups independently selected from C₁-C₆ alkoxy,
      C₃-C₁₀ cycloalkyl,
      —(O)₀₋₁—(C₆-C₁₀ aryl) optionally substituted with 1-3 groups independently selected from C₁-C₆ alkyl,
      3- to 10-membered heterocyclyl, and
      N(R^{N})₂,
   C₁-C₆ alkoxy,
   C₁-C₆ fluoroalkyl,
   C₃-C₁₀ cycloalkyl, and
   3- to 10-membered heterocyclyl.
77. The compound, tautomer, deuterated derivative, or pharmaceutically acceptable salt according to any one of embodiments 66 to 76, wherein each R^{YN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
78. The compound, tautomer, deuterated derivative, or pharmaceutically acceptable salt according to any one of embodiments 66 to 77, wherein each R^{ZN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
79. The compound, tautomer, deuterated derivative, or pharmaceutically acceptable salt according to any one of embodiments 66 to 78, wherein each C(R^{YC})₂ is C═O.
80. The compound, tautomer, deuterated derivative, or pharmaceutically acceptable salt according to any one of embodiments 66 to 79, wherein each C(R^{ZC})₂ is C═O.
81. The compound, tautomer, deuterated derivative, or pharmaceutically acceptable salt according to any one of embodiments 66 to 80, wherein each R^{L1} is independently selected from:
   hydrogen,
   C₁-C₉ alkyl optionally substituted with 1-3 groups independently selected from:
      halogen,
      hydroxyl,
      C₃-C₁₀ cycloalkyl optionally substituted with 1-3 groups independently selected from C₁-C₆ fluoroalkyl,
   C₆-C₁₀ aryl optionally substituted with 1-4 groups independently selected from C₁-C₇ alkyl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
   R^{F}.
82. The compound, tautomer, deuterated derivative, or pharmaceutically acceptable salt according to any one of embodiments 66 to 81, wherein L² is absent.
83. The compound, tautomer, deuterated derivative, or pharmaceutically acceptable salt according to any one of embodiments 66 to 82, wherein each R^{N} is independently selected from C₁-C₈ alkyl optionally substituted with 1-3 groups independently selected from oxo and C₁-C₆ alkoxy.
84. The compound, tautomer, deuterated derivative, or pharmaceutically acceptable salt according to any one of embodiments 66 to 83, wherein when R^{F} is present, two R^{F} taken together with the atoms to which they are bonded form a group selected from:
   C₆-C₁₀ aryl optionally substituted with 1-3 groups independently selected from halogen and C₁-C₆ alkyl, and
   3- to 11-membered heterocyclyl optionally substituted with 1-3 groups independently selected from:
      C₁-C₉ alkyl optionally substituted with 1-4 groups independently selected from:
         —(O)₀₋₁—(C₃-C₁₀ cycloalkyl) optionally substituted with 1-4 groups independently selected from C₁-C₆ alkyl and C₁-C₆ fluoroalkyl,
      C₃-C₁₂ cycloalkyl, and
      C₆-C₁₀ aryl.
85. A compound of Formula III:

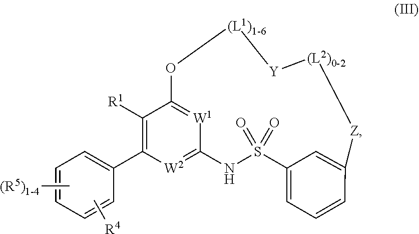

a tautomer thereof, a deuterated derivative of the compound or tautomer, or a pharmaceutically acceptable salt of any of the foregoing, wherein W¹, W², Y, Z, L¹, L², R¹, R⁴, and R⁵ are defined as according to embodiment 1.
86. The compound, tautomer, deuterated derivative, or pharmaceutically acceptable salt according to embodiment 85, wherein W¹ is N and W² is N.
87. The compound, tautomer, deuterated derivative, or pharmaceutically acceptable salt according to embodiment 85 or 86, wherein Y is selected from NR^{YN} and C(R^{YC})₂.
88. The compound, tautomer, deuterated derivative, or pharmaceutically acceptable salt according to any one of embodiments 85 to 87, wherein Z is selected from NR^{ZN}, and C(R^{ZC})₂, provided that when L² is absent, either Y is C(R^{YC})₂ or Z is C(R^{ZC})₂
89. The compound, tautomer, deuterated derivative, or pharmaceutically acceptable salt according to any one of embodiments 85 to 88, wherein Ring C is a phenyl optionally substituted with 1-3 groups independently selected from:
   halogen,
   C₁-C₆ alkyl, and
   N(R^{N})₂.
90. The compound, tautomer, deuterated derivative, or pharmaceutically acceptable salt according to any one of embodiments 85 to 89, wherein R⁴ is selected from hydrogen and methyl.
91. The compound, tautomer, deuterated derivative, or pharmaceutically acceptable salt according to any one of embodiments 85 to 90, wherein R⁴ is methyl.
92. The compound, tautomer, deuterated derivative, or pharmaceutically acceptable salt according to any one of embodiments 85 to 91, wherein each R⁵ is independently selected from:
   hydrogen,
   halogen,
   SO-Me,
   C₁-C₆ alkyl optionally substituted with 1-3 groups independently selected from:
      C₁-C₆ alkoxy optionally substituted with 1-3 groups independently selected from C₁-C₆ alkoxy,
      C₃-C₁₀ cycloalkyl,
      —(O)₀₋₁—(C₆-C₁₀ aryl) optionally substituted with 1-3 groups independently selected from C₁-C₆ alkyl,
      3- to 10-membered heterocyclyl, and
      N(R^{N})₂,
   C₁-C₆ alkoxy,
   C₁-C₆ fluoroalkyl,
   C₃-C₁₀ cycloalkyl, and
   3- to 10-membered heterocyclyl.
93. The compound, tautomer, deuterated derivative, or pharmaceutically acceptable salt according to any one of embodiments 85 to 92, wherein each R^{YN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
94. The compound, tautomer, deuterated derivative, or pharmaceutically acceptable salt according to any one of embodiments 85 to 93, wherein each R^{ZN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
95. The compound, tautomer, deuterated derivative, or pharmaceutically acceptable salt according to any one of embodiments 85 to 94, wherein each C(R^{YC})₂ is C═O.
96. The compound, tautomer, deuterated derivative, or pharmaceutically acceptable salt according to any one of embodiments 85 to 95, wherein each C(R^{ZC})₂ is C═O.
97. The compound, tautomer, deuterated derivative, or pharmaceutically acceptable salt according to any one of embodiments 85 to 96, wherein each R^{L1} is independently selected from:
   hydrogen,
   C₁-C₉ alkyl optionally substituted with 1-3 groups independently selected from:
      halogen,
      hydroxyl, and
      C₃-C₁₀ cycloalkyl optionally substituted with 1-3 groups independently selected from C₁-C₆ fluoroalkyl,
   C₆-C₁₀ aryl optionally substituted with 1-4 groups independently selected from C₁-C₇ alkyl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
   R^{F}.
98. The compound, tautomer, deuterated derivative, or pharmaceutically acceptable salt according to any one of embodiments 85 to 97, wherein L² is absent.
99. The compound, tautomer, deuterated derivative, or pharmaceutically acceptable salt according to any one of embodiments 85 to 98, wherein each R^{N} is independently selected from C₁-C₈ alkyl optionally substituted with 1-3 groups independently selected from oxo and C₁-C₆ alkoxy.
100. The compound, tautomer, deuterated derivative, or pharmaceutically acceptable salt according to any one of embodiments 85 to 99, wherein when R^{F} is present, two R^{F} taken together with the atoms to which they are bonded form a group selected from:
   C₆-C₁₀ aryl optionally substituted with 1-3 groups independently selected from halogen and C₁-C₆ alkyl, and
   3- to 11-membered heterocyclyl optionally substituted with 1-3 groups independently selected from:
      C₁-C₉ alkyl optionally substituted with 1-4 groups independently selected from:
         —(O)₀₋₁—(C₃-C₁₀ cycloalkyl) optionally substituted with 1-4 groups independently selected from C₁-C₆ alkyl and C₁-C₆ fluoroalkyl,
      C₃-C₁₂ cycloalkyl, and
      C₆-C₁₀ aryl.
101. A compound of Formula IV:

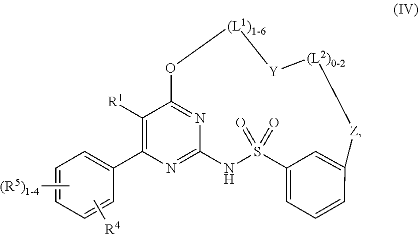

a tautomer thereof, a deuterated derivative of the compound or tautomer, or a pharmaceutically acceptable salt of any of the foregoing, wherein Y, Z, L¹, L², R¹, R⁴, and R⁵ are defined as according to embodiment 1.
102. The compound, tautomer, deuterated derivative, or pharmaceutically acceptable salt according to embodiment 101, wherein Y is selected from NR^{YN} and C(R^{C})₂.
103. The compound, tautomer, deuterated derivative, or pharmaceutically acceptable salt according to embodiment 101 or 102, wherein Z is selected from NR^{ZN}, and C(R^{ZC})₂, provided that when L² is absent, either Y is C(R^{YC})₂ or Z is C(R^{ZC})₂
104. The compound, tautomer, deuterated derivative, or pharmaceutically acceptable salt according to any one of embodiments 101 to 103, wherein Ring C is a phenyl optionally substituted with 1-3 groups independently selected from:
   halogen,
   C₁-C₆ alkyl, and
   N(R^{N})₂.
105. The compound, tautomer, deuterated derivative, or pharmaceutically acceptable salt according to any one of embodiments 101 to 104, wherein R⁴ is selected from hydrogen and methyl.
106. The compound, tautomer, deuterated derivative, or pharmaceutically acceptable salt according to any one of embodiments 101 to 105, wherein R⁴ is methyl.
107. The compound, tautomer, deuterated derivative, or pharmaceutically acceptable salt according to any one of embodiments 101 to 106, wherein each R⁵ is independently selected from:
   hydrogen,
   halogen,
   SO-Me,
   C₁-C₆ alkyl optionally substituted with 1-3 groups independently selected from:
      C₁-C₆ alkoxy optionally substituted with 1-3 groups independently selected from C₁-C₆ alkoxy,
      C₃-C₁₀ cycloalkyl,
      —(O)₀₋₁—(C₆-C₁₀ aryl) optionally substituted with 1-3 groups independently selected from C₁-C₆ alkyl,
      3- to 10-membered heterocyclyl, and
      N(R^{N})₂,
   C₁-C₆ alkoxy,
   C₁-C₆ fluoroalkyl,
   C₃-C₁₀ cycloalkyl, and
   3- to 10-membered heterocyclyl.
108. The compound, tautomer, deuterated derivative, or pharmaceutically acceptable salt according to any one of embodiments 101 to 107, wherein each R^{YN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
109. The compound, tautomer, deuterated derivative, or pharmaceutically acceptable salt according to any one of embodiments 101 to 108, wherein each R^{ZN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
110. The compound, tautomer, deuterated derivative, or pharmaceutically acceptable salt according to any one of embodiments 101 to 109, wherein each C(R^{YC})₂ is C═O.
111. The compound, tautomer, deuterated derivative, or pharmaceutically acceptable salt according to any one of embodiments 101 to 110, wherein each C(R^{ZC})₂ is C═O.
112. The compound, tautomer, deuterated derivative, or pharmaceutically acceptable salt according to any one of embodiments 101 to 111, wherein each R^{L1} is independently selected from:
   hydrogen,
   C₁-C₉ alkyl optionally substituted with 1-3 groups independently selected from:
      halogen,
      hydroxyl, and
      C₃-C₁₀ cycloalkyl optionally substituted with 1-3 groups independently selected from C₁-C₆ fluoroalkyl,
   C₆-C₁₀ aryl optionally substituted with 1-4 groups independently selected from C₁-C₇ alkyl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
   R^{F}.
113. The compound, tautomer, deuterated derivative, or pharmaceutically acceptable salt according to any one of embodiments 101 to 112, wherein L² is absent.
114. The compound, tautomer, deuterated derivative, or pharmaceutically acceptable salt according to any one of embodiments 101 to 113, wherein each R^{N} is independently selected from C₁-C₈ alkyl optionally substituted with 1-3 groups independently selected from oxo and C₁-C₆ alkoxy.
115. The compound, tautomer, deuterated derivative, or pharmaceutically acceptable salt according to any one of embodiments 101 to 114, wherein when R^{F} is present, two R^{F} taken together with the atoms to which they are bonded form a group selected from:
   C₆-C₁₀ aryl optionally substituted with 1-3 groups independently selected from halogen and C₁-C₆ alkyl, and
   3- to 11-membered heterocyclyl optionally substituted with 1-3 groups independently selected from:
      C₁-C₉ alkyl optionally substituted with 1-4 groups independently selected from:
         —(O)₀₋₁—(C₃-C₁₀ cycloalkyl) optionally substituted with 1-4 groups independently selected from C₁-C₆ alkyl and C₁-C₆ fluoroalkyl,
      C₃-C₁₂ cycloalkyl, and
      C₆-C₁₀ aryl.
116. A compound of Formula V:

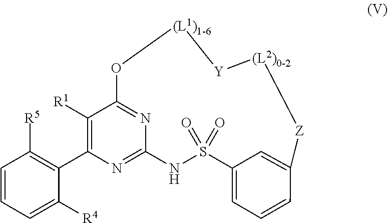

a tautomer thereof, a deuterated derivative of the compound or tautomer, or a pharmaceutically acceptable salt of any of the foregoing, wherein Y, Z, L¹, L², R¹, R⁴, and R⁵ are defined as according to embodiment 1.
117. The compound, tautomer, deuterated derivative, or pharmaceutically acceptable salt according to embodiment 116, wherein Y is selected from NR^{YN} and C(R^{YC})₂.
118. The compound, tautomer, deuterated derivative, or pharmaceutically acceptable salt according to embodiment 116 or 117, wherein Z is selected from NR^{ZN}, and C(R^{ZC})₂, provided that when L² is absent, either Y is C(R^{YC})₂ or Z is C(R^{ZC})₂
119. The compound, tautomer, deuterated derivative, or pharmaceutically acceptable salt according to any one of embodiments 116 to 118, wherein Ring C is a phenyl optionally substituted with 1-3 groups independently selected from:
   halogen,
   C₁-C₆ alkyl, and
   N(R^{N})₂.
120. The compound, tautomer, deuterated derivative, or pharmaceutically acceptable salt according to any one of embodiments 116 to 119, wherein R⁴ is selected from hydrogen and methyl.
121. The compound, tautomer, deuterated derivative, or pharmaceutically acceptable salt according to any one of embodiments 116 to 120, wherein R⁴ is methyl.
122. The compound, tautomer, deuterated derivative, or pharmaceutically acceptable salt according to any one of embodiments 116 to 121, wherein each R⁵ is independently selected from:
   hydrogen,
   halogen,
   SO-Me,
   C₁-C₆ alkyl optionally substituted with 1-3 groups independently selected from:
      C₁-C₆ alkoxy optionally substituted with 1-3 groups independently selected from C₁-C₆ alkoxy,
      C₃-C₁₀ cycloalkyl,
      —(O)₀₋₁—(C₆-C₁₀ aryl) optionally substituted with 1-3 groups independently selected from C₁-C₆ alkyl,
      3- to 10-membered heterocyclyl, and
      N(R^{N})₂,
   C₁-C₆ alkoxy,
   C₁-C₆ fluoroalkyl,
   C₃-C₁₀ cycloalkyl, and
   3- to 10-membered heterocyclyl.
123. The compound, tautomer, deuterated derivative, or pharmaceutically acceptable salt according to any one of embodiments 116 to 122, wherein each R^{YN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
124. The compound, tautomer, deuterated derivative, or pharmaceutically acceptable salt according to any one of embodiments 116 to 123, wherein each R^{ZN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
125. The compound, tautomer, deuterated derivative, or pharmaceutically acceptable salt according to any one of embodiments 116 to 124, wherein each C(R^{YC})₂ is C═O.
126. The compound, tautomer, deuterated derivative, or pharmaceutically acceptable salt according to any one of embodiments 116 to 125, wherein each C(R^{ZC})₂ is C═O.
127. The compound, tautomer, deuterated derivative, or pharmaceutically acceptable salt according to any one of embodiments 116 to 126, wherein each R^{L1} is independently selected from:
   hydrogen,
   C₁-C₉ alkyl optionally substituted with 1-3 groups independently selected from:
      halogen,
      hydroxyl,
      C₃-C₁₀ cycloalkyl optionally substituted with 1-3 groups independently selected from C₁-C₆ fluoroalkyl,
   C₆-C₁₀ aryl optionally substituted with 1-4 groups independently selected from C₁-C₇ alkyl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
   R^{F}.
128. The compound, tautomer, deuterated derivative, or pharmaceutically acceptable salt according to any one of embodiments 116 to 127, wherein L² is absent.
129. The compound, tautomer, deuterated derivative, or pharmaceutically acceptable salt according to any one of embodiments 116 to 128, wherein each R^{N} is independently selected from C₁-C₈ alkyl optionally substituted with 1-3 groups independently selected from oxo and C₁-C₆ alkoxy.
130. The compound, tautomer, deuterated derivative, or pharmaceutically acceptable salt according to any one of embodiments 116 to 129, wherein when R^{F} is present, two R^{F} taken together with the atoms to which they are bonded form a group selected from:
   C₆-C₁₀ aryl optionally substituted with 1-3 groups independently selected from halogen and C₁-C₆ alkyl, and
   3- to 11-membered heterocyclyl optionally substituted with 1-3 groups independently selected from:
      C₁-C₉ alkyl optionally substituted with 1-4 groups independently selected from:
         —(O)₀₋₁—(C₃-C₁₀ cycloalkyl) optionally substituted with 1-4 groups independently selected from C₁-C₆ alkyl and C₁-C₆ fluoroalkyl,
      C₃-C₁₂ cycloalkyl, and
      C₆-C₁₀ aryl.
131. A compound of Formula VIa:

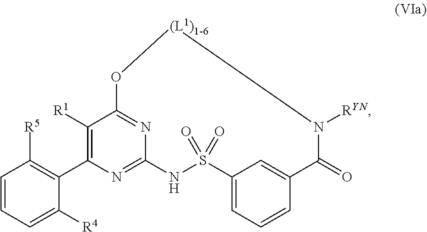

a tautomer thereof, a deuterated derivative of the compound or tautomer, or a pharmaceutically acceptable salt of any of the foregoing, wherein L¹, R¹, R⁴, R⁵, and R^{YN} are defined as according to embodiment 1.
132. The compound, tautomer, deuterated derivative, or pharmaceutically acceptable salt according to embodiment 131, wherein Ring C is a phenyl optionally substituted with 1-3 groups independently selected from:
   halogen,
   C₁-C₆ alkyl, and
   N(R^{N})₂.
133. The compound, tautomer, deuterated derivative, or pharmaceutically acceptable salt according to embodiment 131 or 132, wherein R⁴ is selected from hydrogen and methyl.
134. The compound, tautomer, deuterated derivative, or pharmaceutically acceptable salt according to any one of embodiments 131 to 133, wherein R⁴ is methyl.
135. The compound, tautomer, deuterated derivative, or pharmaceutically acceptable salt according to any one of embodiments 131 to 134, wherein each R⁵ is independently selected from:
   hydrogen,
   halogen,
   SO-Me,
   C₁-C₆ alkyl optionally substituted with 1-3 groups independently selected from:
      C₁-C₆ alkoxy optionally substituted with 1-3 groups independently selected from C₁-C₆ alkoxy,
      C₃-C₁₀ cycloalkyl,
      —(O)₀₋₁—(C₆-C₁₀ aryl) optionally substituted with 1-3 groups independently selected from C₁-C₆ alkyl,
      3- to 10-membered heterocyclyl, and
      N(R^{N})₂,
   C₁-C₆ alkoxy,
   C₁-C₆ fluoroalkyl,
   C₃-C₁₀ cycloalkyl, and
   3- to 10-membered heterocyclyl.
136. The compound, tautomer, deuterated derivative, or pharmaceutically acceptable salt according to any one of embodiments 131 to 135, wherein each R^{YN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
137. The compound, tautomer, deuterated derivative, or pharmaceutically acceptable salt according to any one of embodiments 131 to 136, wherein each R^{L1} is independently selected from:
   hydrogen,
   C₁-C₉ alkyl optionally substituted with 1-3 groups independently selected from:
      halogen,
      hydroxyl, and
      C₃-C₁₀ cycloalkyl optionally substituted with 1-3 groups independently selected from C₁-C₆ fluoroalkyl,
   C₆-C₁₀ aryl optionally substituted with 1-4 groups independently selected from C₁-C₇ alkyl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
   R^{F}.
138. The compound, tautomer, deuterated derivative, or pharmaceutically acceptable salt according to any one of embodiments 131 to 137, wherein each R^{N} is independently selected from C₁-C₈ alkyl optionally substituted with 1-3 groups independently selected from oxo and C₁-C₆ alkoxy.
139. The compound, tautomer, deuterated derivative, or pharmaceutically acceptable salt according to any one of embodiments 131 to 138, wherein when R^{F} is present, two R^{F} taken together with the atoms to which they are bonded form a group selected from:
   C₆-C₁₀ aryl optionally substituted with 1-3 groups independently selected from halogen and C₁-C₆ alkyl, and
   3- to 11-membered heterocyclyl optionally substituted with 1-3 groups independently selected from:
      C₁-C₉ alkyl optionally substituted with 1-4 groups independently selected from:
         —(O)₀₋₁—(C₃-C₁₀ cycloalkyl) optionally substituted with 1-4 groups independently selected from C₁-C₆ alkyl and C₁-C₆ fluoroalkyl,
      C₃-C₁₂ cycloalkyl, and
      C₆-C₁₀ aryl.
140. A compound of Formula VIb:

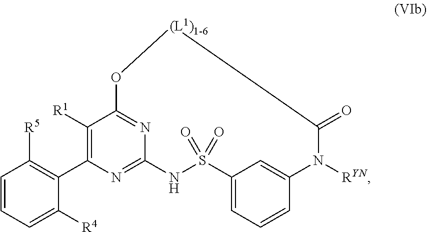

a tautomer thereof, a deuterated derivative of the compound or tautomer, or a pharmaceutically acceptable salt of any of the foregoing, wherein L¹, R¹, R⁴, R⁵, and R^{ZN} are defined as according to embodiment 1.
141. The compound, tautomer, deuterated derivative, or pharmaceutically acceptable salt according to embodiment 140, wherein Ring C is a phenyl optionally substituted with 1-3 groups independently selected from:
   halogen,
   C₁-C₆ alkyl, and
   N(R^{N})₂.
142. The compound, tautomer, deuterated derivative, or pharmaceutically acceptable salt according to embodiment 140 or 141, wherein R⁴ is selected from hydrogen and methyl.
143. The compound, tautomer, deuterated derivative, or pharmaceutically acceptable salt according to any one of embodiments 140 to 142, wherein R⁴ is methyl.
144. The compound, tautomer, deuterated derivative, or pharmaceutically acceptable salt according to any one of embodiments 140 to 143, wherein each R⁵ is independently selected from:
   hydrogen,
   halogen,
   SO-Me,
   C₁-C₆ alkyl optionally substituted with 1-3 groups independently selected from:
      C₁-C₆ alkoxy optionally substituted with 1-3 groups independently selected from C₁-C₆ alkoxy,
      C₃-C₁₀ cycloalkyl,
      —(O)₀₋₁—(C₆-C₁₀ aryl) optionally substituted with 1-3 groups independently selected from C₁-C₆ alkyl,
      3- to 10-membered heterocyclyl, and
      N(R^{N})₂,
   C₁-C₆ alkoxy,
   C₁-C₆ fluoroalkyl,
   C₃-C₁₀ cycloalkyl, and
   3- to 10-membered heterocyclyl.
145. The compound, tautomer, deuterated derivative, or pharmaceutically acceptable salt according to any one of embodiments 140 to 144, wherein each R^{ZN} is independently selected from:
   hydrogen,
   C₁-C₉ alkyl optionally substituted with 1-3 groups independently selected from:
      hydroxyl,
      oxo,
      C₆-C₁₀ aryl optionally substituted with 1-3 groups independently selected from C₁-C₆ alkyl (optionally substituted with 1-3 hydroxyl), and
      5- to 10-membered heteroaryl optionally substituted with 1-3 groups independently selected from:
         N(R^{N})₂,
         C₁-C₆ alkyl,
         C₁-C₆ alkoxy,
         —(O)₀₋₁—(C₃-C₁₀ cycloalkyl), and
         —(O)₀₋₁-(3- to 10-membered heterocyclyl) optionally substituted with 1-4 groups independently selected from C₁-C₆ alkyl,
   C₃-C₁₀ cycloalkyl optionally substituted with 1-3 groups independently selected from:
      hydroxyl,
      C₁-C₆ alkyl optionally substituted with 1-3 groups independently selected from hydroxyl, and
      C₁-C₆ alkoxy,
   3- to 10-membered heterocyclyl optionally substituted with 1-3 groups independently selected from C₁-C₆ alkyl (optionally substituted with 1-3 groups independently selected from oxo and C₁-C₆ alkoxy),
   5- to 10-membered heteroaryl optionally substituted with 1-3 groups independently selected from halogen and C₁-C₆ alkyl, and
   R^{F}.
146. The compound, tautomer, deuterated derivative, or pharmaceutically acceptable salt according to any one of embodiments 140 to 145, wherein each R^{L1} is independently selected from:
   hydrogen,
   C₁-C₉ alkyl optionally substituted with 1-3 groups independently selected from:
      halogen,
      hydroxyl, and
      C₃-C₁₀ cycloalkyl optionally substituted with 1-3 groups independently selected from C₁-C₆ fluoroalkyl,
   C₆-C₁₀ aryl optionally substituted with 1-4 groups independently selected from C₁-C₇ alkyl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
   R^{F}.
147. The compound, tautomer, deuterated derivative, or pharmaceutically acceptable salt according to any one of embodiments 140 to 146, wherein each R^{N} is independently selected from C₁-C₈ alkyl optionally substituted with 1-3 groups independently selected from oxo and C₁-C₆ alkoxy.
148. The compound, tautomer, deuterated derivative, or pharmaceutically acceptable salt according to any one of embodiments 140 to 147, wherein when R^{F} is present, two R^{F} taken together with the atoms to which they are bonded form a group selected from:
   C₆-C₁₀ aryl optionally substituted with 1-3 groups independently selected from halogen and C₁-C₆ alkyl, and
   3- to 11-membered heterocyclyl optionally substituted with 1-3 groups independently selected from:
      C₁-C₉ alkyl optionally substituted with 1-4 groups independently selected from:
         —(O)₀₋₁—(C₃-C₁₀ cycloalkyl) optionally substituted with 1-4 groups independently selected from C₁-C₆ alkyl and C₁-C₆ fluoroalkyl,
      C₃-C₁₂ cycloalkyl, and
      C₆-C₁₀ aryl.
149. The compound, tautomer, deuterated derivative, or pharmaceutically acceptable salt according to any one of embodiments 1 to 148, selected from compounds of any one of Formulae I, Ia, IIa, IIb, III, IV, V, VIa, and VIb, tautomers thereof, deuterated derivatives of those compounds and tautomers, and pharmaceutically acceptable salts of any of the foregoing.
150. The compound, tautomer, deuterated derivative, or pharmaceutically acceptable salt according to any one of embodiments 1 to 149, selected from Compounds 1-320 (Tables 3-7, 9, 11, 13, 15, 17, 20, and 21) Compounds 321-330 (Table 19), Compounds 331-364 (Table 22-24), and Compounds 365-369 (Tables 25-27), deuterated derivatives thereof and pharmaceutically acceptable salts of any of the foregoing.
151. A pharmaceutical composition comprising the compound, tautomer, deuterated derivative, or pharmaceutically acceptable salt according to any one of embodiments 1 to 150, and a pharmaceutically acceptable carrier.
152. The pharmaceutical composition of embodiment 151, further comprising one or more additional therapeutic agents.
153. The pharmaceutical composition of embodiment 152, wherein the one or more additional therapeutic agents is selected from mucolytic agents, bronchodilators, antibiotics, anti-infective agents, and anti-inflammatory agents.
154. The pharmaceutical composition of embodiment 153, wherein the one or more additional therapeutic agent is an antibiotic selected from tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.
155. The pharmaceutical composition of embodiment 152, wherein the one or more additional therapeutic agent is one or more CFTR modulating agents.
156. The pharmaceutical composition of embodiment 155, wherein the one or more CFTR modulating agents are selected from CFTR potentiators.
157. The pharmaceutical composition of embodiment 155, wherein the one or more CFTR modulating agents are selected from CFTR correctors.
158. The pharmaceutical composition of embodiment 155, wherein the one or more CFTR modulating agents comprises at least one CFTR potentiator and at least one CFTR corrector.
159. The pharmaceutical composition of any one of embodiment 155-158, wherein the one or more CFTR modulating agents are selected from (a) tezacaftor, lumacaftor, and deuterated derivatives and pharmaceutically acceptable salts thereof; and (b) ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.
160. The pharmaceutical composition of any one of embodiment 155-158, wherein the one or more CFTR modulating agents are selected from (a) tezacaftor, lumacaftor, and deuterated derivatives and pharmaceutically acceptable salts thereof; or (b) ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.
161. The pharmaceutical composition of any one of embodiment 155-159, wherein the composition comprises tezacaftor and ivacaftor.
162. The pharmaceutical composition of any one of embodiment 155-159, wherein the composition comprises tezacaftor and deutivacaftor.
163. The pharmaceutical composition of any one of embodiment 155-159, wherein the composition comprises tezacaftor and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol.
164. The pharmaceutical composition of any one of embodiment 155-159, wherein the composition comprises lumacaftor and ivacaftor.
165. The pharmaceutical composition of any one of embodiment 155-159, wherein the composition comprises lumacaftor and deutivacaftor.
166. The pharmaceutical composition of any one of embodiment 155-159, wherein the composition comprises lumacaftor and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol.
167. A method of treating cystic fibrosis comprising administering to a patient in need thereof the compound, tautomer, deuterated derivative, or pharmaceutically acceptable salt according to any one of embodiments 1 to 152, or a pharmaceutical composition according to any one of embodiments 151 to 166.
168. The method of embodiment 167, comprising administering to the patient in need thereof the compound, tautomer, deuterated derivative, or pharmaceutically acceptable salt according to any one of embodiments 1 to 150, or a pharmaceutical composition according to embodiment 151, and further administrating one or more additional therapeutic agents prior to, concurrent with, or subsequent to the compound, tautomer, deuterated derivative, or pharmaceutically acceptable salt according to any one of embodiments 1 to 150, or the pharmaceutical composition according to embodiment 151.
169. The method of embodiment 168, wherein the one or more additional therapeutic agents is(are) selected from CFTR modulating agents.
170. The method of embodiment 169, wherein the one or more CFTR modulating agents are selected from CFTR potentiators.
171. The method of embodiment 169, wherein the one or more CFTR modulating agents are selected from CFTR correctors.
172. The method of embodiment 169, wherein the one or more CFTR modulating agents comprising both a CFTR potentiator and an additional CFTR corrector.
173. The method of embodiment 170 and 172, wherein the CFTR potentiator is selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.
174. The method of embodiment 171 or embodiment 172, wherein the CFTR corrector is selected from tezacaftor, lumacaftor, and deuterated derivatives and pharmaceutically acceptable salts thereof.
175. The method of any one of embodiments 167 to 174, comprising administration of CFTR modulating agents ivacaftor and tezacaftor.
176. The method of any one of embodiments 167 to 174, comprising administration of CFTR modulating agents deutivacaftor and tezacaftor.
177. The method of any one of embodiments 167 to 174, comprising administration of CFTR modulating agents (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and tezacaftor.
178. The method of any one of embodiments 167 to 174, comprising administration of CFTR modulating agents ivacaftor and lumacaftor.
179. The method of any one of embodiments 167 to 174, comprising administration of CFTR modulating agents deutivacaftor and lumacaftor.
180. The method of any one of embodiments 167 to 174, comprising administration of CFTR modulating agents (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and lumacaftor.
181. The method of embodiment 169, wherein the one or more additional therapeutic agents is one or more compounds selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof.
182. The compound, tautomer, deuterated derivative, or pharmaceutically acceptable salt according to any one of embodiments 1 to 150, or the pharmaceutical composition according to any one of embodiments 151 to 166 for use in the treatment of cystic fibrosis.
183. The compound, tautomer, deuterated derivative, or pharmaceutically acceptable salt according to any one of embodiments 1 to 150, or the pharmaceutical composition according to any one of embodiments 151 to 166 for use in the manufacture of a medicament for the treatment of cystic fibrosis.
184. A compound selected from Compounds 1-369, tautomers thereof, deuterated derivatives of those compounds and tautomers, and pharmaceutically acceptable salts of any of the foregoing.
185. A deuterated derivative of a compound selected from Compounds 1-369.
186. A pharmaceutically acceptable salt of a compound selected from Compounds 1-369.
187. A compound selected from Compounds 1-369.
188. A pharmaceutical composition comprising a compound selected from Compounds 1-369, tautomers thereof, deuterated derivatives of those compounds and tautomers, and pharmaceutically acceptable salts of any of the foregoing and a pharmaceutically acceptable carrier.
189. A pharmaceutical composition comprising a deuterated derivative of a compound selected from Compounds 1-369 and a pharmaceutically acceptable carrier.
190. A pharmaceutical composition comprising a pharmaceutically acceptable salt of a compound selected from Compounds 1-369 and a pharmaceutically acceptable carrier.
191. A pharmaceutical composition comprising a compound selected from Compounds 1-369 and a pharmaceutically acceptable carrier.
192. A pharmaceutical composition comprising (a) a compound selected from Compounds 1-369, tautomers thereof, deuterated derivatives of those compounds and tautomers, and pharmaceutically acceptable salts of any of the foregoing; (b) a CFTR potentiator; and (c) a pharmaceutically acceptable carrier.
193. A pharmaceutical composition comprising (a) a deuterated derivative of a compound selected from Compounds 1-369; (b) a CFTR potentiator; and (c) a pharmaceutically acceptable carrier.
194. A pharmaceutical comprising (a) a pharmaceutically acceptable salt of a compound selected from Compounds 1-369; (b) a CFTR potentiator; and (c) a pharmaceutically acceptable carrier.
195. A pharmaceutical composition comprising (a) a compound selected from Compounds 1-369; (b) a CFTR potentiator; and (c) a pharmaceutically acceptable carrier.
196. A pharmaceutical composition comprising (a) a compound selected from Compounds 1-369, tautomers thereof, deuterated derivatives of those compounds and tautomers, and pharmaceutically acceptable salts of any of the foregoing; (b) an additional CFTR corrector; and (c) a pharmaceutically acceptable carrier.
197. A pharmaceutical composition comprising (a) a deuterated derivative of a compound selected from Compounds 1-369; (b) an additional CFTR corrector; and (c) a pharmaceutically acceptable carrier.
198. A pharmaceutical composition comprising (a) a pharmaceutically acceptable salt of a compound selected from Compounds 1-369; (b) an additional CFTR corrector; and (c) a pharmaceutically acceptable carrier.
199. A pharmaceutical composition comprising (a) a compound selected from Compounds 1-369; (b) an additional CFTR corrector; and (c) a pharmaceutically acceptable carrier.
200. A pharmaceutical composition comprising (a) a compound selected from Compounds 1-369, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier.
201. A pharmaceutical composition comprising (a) a deuterated derivative of a compound selected from Compounds 1-369; (b) an additional CFTR corrector; (c) a CFTR potentiator; and (d) a pharmaceutically acceptable carrier.
202. A pharmaceutical composition comprising (a) a pharmaceutically acceptable salt of a compound selected from Compounds 1-369; (b) an additional CFTR corrector; (c) a CFTR potentiator; and (d) a pharmaceutically acceptable carrier.
203. A pharmaceutical composition comprising (a) a compound selected from Compounds 1-369; (b) an additional CFTR corrector; (c) a CFTR potentiator; and (d) a pharmaceutically acceptable carrier.
204. A compound selected from Compounds 1-369, tautomers thereof, deuterated derivatives of those compounds and tautomers, and pharmaceutically acceptable salts of any of the foregoing for use in a method of treating cystic fibrosis.
205. A deuterated derivative of a compound selected from Compounds 1-369 for use in a method of treating cystic fibrosis.
206. A pharmaceutically acceptable salt of a compound selected from Compounds 1-369 for use in a method of treating cystic fibrosis.
207. A compound selected from Compounds 1-369 for use in a method of treating cystic fibrosis.
208. A pharmaceutical composition comprising a compound selected from Compounds 1-369, tautomers thereof, deuterated derivatives of those compounds and tautomers, and pharmaceutically acceptable salts of any of the foregoing and a pharmaceutically acceptable carrier for use in a method of treating cystic fibrosis.
209. A pharmaceutical composition comprising a deuterated derivative of a compound selected from Compounds 1-369 and a pharmaceutically acceptable carrier for use in a method of treating cystic fibrosis.
210. A pharmaceutical composition comprising a pharmaceutically acceptable salt of a compound selected from Compounds 1-369 and a pharmaceutically acceptable carrier for use in a method of treating cystic fibrosis.
211. A pharmaceutical composition comprising a compound selected from Compounds 1-369 and a pharmaceutically acceptable carrier for use in a method of treating cystic fibrosis.
212. A pharmaceutical composition comprising (a) a compound selected from Compounds 1-369, tautomers thereof, deuterated derivatives of those compounds and tautomers, and pharmaceutically acceptable salts of any of the foregoing; (b) a CFTR potentiator; and (c) a pharmaceutically acceptable carrier for use in a method of treating cystic fibrosis.
213. A pharmaceutical comprising (a) a deuterated derivative of a compound selected from Compounds 1-369; (b) a CFTR potentiator; and (c) a pharmaceutically acceptable carrier for use in a method of treating cystic fibrosis.
214. A pharmaceutical composition comprising (a) a pharmaceutically acceptable salt of a compound selected from Compounds 1-369; (b) a CFTR potentiator; and (c) a pharmaceutically acceptable carrier for use in a method of treating cystic fibrosis.
215. A pharmaceutical composition comprising (a) a compound selected from Compounds 1-369; (b) a CFTR potentiator; and (c) a pharmaceutically acceptable carrier.
216. A pharmaceutical composition comprising (a) a compound selected from Compounds 1-369, tautomers thereof, deuterated derivatives of those compounds and tautomers, and pharmaceutically acceptable salts of any of the foregoing; (b) an additional CFTR corrector; and (c) a pharmaceutically acceptable carrier for use in a method of treating cystic fibrosis.
217. A pharmaceutical composition comprising (a) a deuterated derivative of a compound selected from Compounds 1-369; (b) an additional CFTR corrector; and (c) a pharmaceutically acceptable carrier for use in a method of treating cystic fibrosis.
218. A pharmaceutical composition comprising (a) a pharmaceutically acceptable salt of a compound selected from Compounds 1-369; (b) an additional CFTR corrector; and (c) a pharmaceutically acceptable carrier for use in a method of treating cystic fibrosis.
219. A pharmaceutical composition comprising (a) a compound selected from Compounds 1-369; (b) an additional CFTR corrector; and (c) a pharmaceutically acceptable carrier for use in a method of treating cystic fibrosis.
220. A pharmaceutical composition comprising (a) a compound selected from Compounds 1-369, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier for use in a method of treating cystic fibrosis.
221. A pharmaceutical composition comprising (a) a deuterated derivative of a compound selected from Compounds 1-369; (b) an additional CFTR corrector; (c) a CFTR potentiator; and (d) a pharmaceutically acceptable carrier for use in a method of treating cystic fibrosis.
222. A pharmaceutical composition comprising (a) a pharmaceutically acceptable salt of a compound selected from Compounds 1-369; (b) an additional CFTR corrector; (c) a CFTR potentiator; and (d) a pharmaceutically acceptable carrier for use in a method of treating cystic fibrosis.
223. A pharmaceutical composition comprising (a) a compound selected from Compounds 1-369; (b) an additional CFTR corrector; (c) a CFTR potentiator; and (d) a pharmaceutically acceptable carrier for use in a method of treating cystic fibrosis.

### EXAMPLES

### I. Abbreviation List

ACN: Acetonitrile
Boc anhydride ((Boc)₂O): Di-tert-butyl decarbonate
CDCl₃: Chloroform-d CDI: Carbonyl diimidazoleCDI: Carbonyl diimidazole
CDMT: 2-Chloro-4,6-dimethoxy-1,3,5-triazine
CH₂C₁₂: Dichloromethane
CH₃CN: Acetonitrile
COMU: (1-Cyano-2-ethoxy-2-oxoethylidenaminooxy)dimethylamino-morpholino-carbenium
hexafluorophosphate
Cmpd: Compound
DABCO: 1,4-Diazabicyclo[2.2.2]octane
DBU: 1,8-Diazabicyclo(5.4.0)undec-7-ene
DCE: 1,2-Dichloroethane
DCM: Dichloromethane
DI: Deionized
DIAD: Diisopropyl azodicarboxylate
DIBAL, DIBALH: Diisobutylaluminium hydride
DIEA: (DIPEA, DiPEA): N,N-diisopropylethylamine
DMA: N,N-Dimethylacetamide
DMAP: 4-Dimethylaminopyridine
DMF: N,N-Dimethylformamide
DMSO: Dimethyl sulfoxide
DMP: Dess-Martin periodinane
EA: Ethyl acetate
EDC: 1-Ethyl-3-(3-dimethylaminopropyl)carbodiimide
ELSD: Evaporative light scattering detector
ESI-MS: Electrospray ionization mass spectrometry
EtOAc: Ethyl acetate
EtOH: Ethanol
GC: Gas chromatography
Grubbs 1^{st} Generation catalyst: Dichloro(benzylidene)bis(tricyclohexylphosphine)ruthenium(II)
Grubbs 2^{nd} Generation catalyst: [1,3-Bis(2,4,6-trimethylphenyl)imidazolidin-2-ylidene]-dichloro-[(2-isopropoxyphenyl)methylene]ruthenium
HATU: 1-[Bis(dimethylamino)methylene]-1H-1,2,3-triazolo[4,5-b]pyridinium 3-oxid hexafluorophosphate
HPLC: High-performance liquid chromatography
Hoveyda-Grubbs 2^{nd} Generation catalyst: (1,3-Bis-(2,4,6-trimethylphenyl)-2-imidazolidinylidene)dichloro(o-isopropoxyphenylmethylene)ruthenium, Dichloro[1,3-bis(2,4,6-trimethylphenyl)-2-imidazolidinylidene](2-isopropoxyphenylmethylene)ruthenium(II)
IPA: Isopropanol
KHSO₄: Potassium bisulfate
LC: Liquid chromatography
LCMS: Liquid chromatography mass spectrometry
LCMS Met.: LCMS method
LCMS Rt: LCMS retention time
LDA: Lithium diisopropylamide
LiOH: Lithium hydroxide
MeCN: Acetonitrile
MeOH: Methanol
MTBE: Methyl tert-butyl ether
MeTHF or 2-MeTHF: 2-Methyltetrahydrofuran
MgSO₄: Magnesium sulfate
NaHCO₃: Sodium bicarbonate
NaOH: Sodium hydroxide
NMP: N-Methyl-2-pyrrolidone
NMM: N-Methylmorpholine
Pd/C: Palladium on carbon
Pd₂(dba)₃: Tris(dibenzylideneacetone)dipalladium(0)
Pd(dppf)Cl₂: [1,1′-Bis(diphenylphosphino)ferrocene]dichloropalladium(II)
Pd(OAc)₂: Palladium(II) acetate
PTFE: Polytetrafluoroethylene
rt, RT: Room temperature
RuPhos: 2-Dicyclohexylphosphino-2′,6′-diisopropoxybiphenyl
SFC: Supercritical fluid chromatography
SM: starting material
TBAI: Tetrabutylammonium iodide
TEA: Triethylamine
TFA: Trifluoroacetic acid
THF: Tetrahydrofuran
TLC: Thin layer chromatography
TMS: Trimethylsilyl
TMSCl: Trimethylsilyl chloride
T3P: Propanephosphonic acid anhydride
UPLC: Ultra Performance Liquid Chromatography
XANTPHOS: 4,5-Bis(diphenylphosphino)-9,9-dimethylxanthene
XPhos: 2-Dicyclohexylphosphino-2′,4′,6′-triisopropylbiphenyl

### II. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32k points and a line broadening of 0.3 Hz was applied before Fourier transform. 19F NMR spectra were recorded at 282 MHz using a 30 degree pulse angle, a spectral width of 100 kHz and 59202 points were acquired. FID were zero-filled to 64k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128k points of acquisition. FID were zero-filled to 256k points and a line broadening of 0.3 Hz was applied before fourrier transform. 19F NMR spectra were recorded at 377 MHz using a 30 deg pulse angle, a spectral width of 89286 Hz and 128k points were acquired. FID were zero-filled to 256k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Final purity of compounds was determined by reversed phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range. Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30 m×0.25 mm×0.25 μm_df) column, with a 2.0 mL/min flow rate (H₂ carrier gas), at an injection temperature of 220° C. and an oven temperature of 120° C., 15 minutes.

### III. General UPLC/HPLC Analytical Methods

LC method A: Analytical reverse phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 L, and column temperature=60° C.

LC method B: Reverse phase HPLC using a Kinetex C₁₈ column (50×3.0 mm) and a dual gradient run from 5-100% mobile phase B over 6 minutes. Mobile phase A=H₂O (0.1% CF₃CO₂H). Mobile phase B=CH₃CN (0.1% CF₃CO₂H). Flow rate=1.5 mL/min, injection volume=2 μL, and column temperature=60° C.

LC method C: Kinetex C₁₈ 4.6×50 mm 2.6 μm. Temp: 45° C., Flow: 2.0 mL/min, Run Time: 3 min. Mobile phase: Initial 95% water (0.1% formic acid) and 5% acetonitrile (0.1% formic acid) linear gradient to 95% acetonitrile (0.1% formic acid) for 2.0 min then hold at 95% acetonitrile (0.1% formic acid) for 1.0 min.

LC method D: Acquity UPLC BEH C₁₈ column (30×2.1 mm, 1.7 μm particle) made by Waters (pn: 186002349), and a dual gradient run from 1-99% mobile phase B over 1.0 minute. Mobile phase A=H₂O (0.05% CF₃CO₂H). Mobile phase B=CH₃CN (0.035% CF₃CO₂H). Flow rate=1.5 mL/min, injection volume=1.5 μL, and column temperature=60° C.

LC method G: Symmetry, 4.6×75 mm 3.5 μm. Temp: 45° C., Flow: 2.0 mL/min, Run Time: 8 min. Mobile Phase: Initial 95% H₂O (0.11% Formic Acid) and 5% CH₃CN (0.1% FA) linear gradient to 95% CH₃CN (0.1% formic acid) for 6.0 min then hold at 95% CH₃CN (0.1% formic acid) for 2.0 min.

LC method I: Acquity UPLC BEH C₁₈ column (50×2.1 mm, 1.7 μm particle) made by Waters (pn:186002350), and a dual gradient run from 1-99% mobile phase B over 5.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method K: Kinetex Polar C₁₈ 3.0×50 mm 2.6 μm, 3 min, 5-95% ACN in H₂O (0.1% Formic Acid) 1.2 ml/min.

LC method P: Poroshell 120 EC-C18 3.0×50 mm 2.7 μM, Temp:45° C., Flow: 1.5 mL/min, RunTime: 3 min. Mobile phase conditions: Initial. 95% H₂O (0.1% Formic Acid) and 5% CH₃CN (0.1% FA) linear gradient to 95% CH₃CN (0.1% FA) for 1.5 min then hold at 95% CH₃CN (0.1% FA) for 1.5 min.

LC method S: Merckmillipore Chromolith SpeedROD C₁₈ column (50×4.6 mm) and a dual gradient run from 5-100% mobile phase B over 12 minutes. Mobile phase A=water (0.1% CF₃CO₂H). Mobile phase B=acetonitrile (0.1% CF₃CO₂H).

LC method T: Merckmillipore Chromolith SpeedROD C₁₈ column (50×4.6 mm) and a dual gradient run from 5-100% mobile phase B over 6 minutes. Mobile phase A=water (0.1% CF₃CO₂H). Mobile phase B=acetonitrile (0.1% CF₃CO₂H).

LC method U: Kinetex Polar C₁₈ 3.0×50 mm 2.6 m, 6 min, 5-95% ACN in H₂O (0.1% Formic Acid) 1.2 mL/min.

LC method V: Acquity UPLC BEH C₁₈ column (50×2.1 mm, 1.7 μm particle) made by Waters (pn: 186002350), and a dual gradient run from 1-30% mobile phase B over 2.9 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method W: water Cortex 2.7μ C₁₈ (3.0 mm×50 mm), Temp: 55° C.; Flow: 1.2 mL/min; mobile phase: 100% water with 0.1% trifluoroacetic (TFA) acid then 100% acetonitrile with 0.1% TFA acid, grad:5% to 100% B over 4 min, with stay at 100% B for 0.5 min, equilibration to 5% B over 1.5 min.

LC method X: UPLC Luna C₁₈(2) 50×3 mm 3 μm. run: 2.5 min. Mobile phase: Initial 95% H₂O 0.1% FA/5% MeCN 0.1% FA, linear grad to 95% MeCN 0.1% FA over 1.3 min, hold 1.2 min 95% CH₃CN 0.1% FA, T: 45C, Flow: 1.5 mL/min

LC method Y: UPLC SunFire C₁₈ 75×4.6 mm 3.5 m, run: 6 min. Mobile phase conditions: Initial 95% H₂O+0.1% FA/5% CH₃CN+0.1% FA, linear gradient to 95% CH₃CN for 4 min, hold for 2 min at 95% CH₃CN. T:45° C., Flow:1.5 mL/min

LC method 1D: XBridge C₁₈ 4.6×75 mm, 5 m, Initial Gradient at 95% NH₄HCO₃/5% MeCN 6 min run with 1 min equilibration gradient 0 to 3 min at 95% MeCN and hold for 3 minutes. Flow 1.5 mL/min.

LC method 1E: SunFire C₁₈ 4.6×75 mm, 5 μM, 6 min run, 50-95% ACN/Water (0.1% formic acid modifier), 1.5 min equilibration, gradient over 3 min, hold 3 min. 1.5 mL/min

### IV. Synthesis of Common Intermediates

### Example A: Preparation of 4-chloro-6-(2,6-dimethylphenyl)pyrimidin-2-amine

### Step 1: tert-Butyl N-tert-butoxycarbonyl-N-(4,6-dichloropyrimidin-2-yl)carbamate

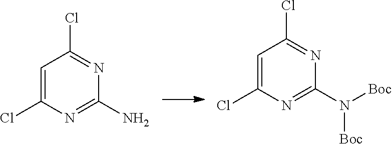 [SMILES: Nc1nc(Cl)cc(Cl)n1 ; CC(C)(C)OC(=O)N(C(=O)OC(C)(C)C)c1nc(Cl)cc(Cl)n1]

To a solution of 4,6-dichloropyrimidin-2-amine (300 g, 1.829 mol) in DCM (2.1 L) was added (BOC)₂O (838 g, 3.840 mol) followed by DMAP (5.6 g, 45.84 mmol). The mixture was stirred at ambient temperature for 6 h. Additional DMAP (5.6 g, 45.84 mmol) was added and the reaction was continued to stir at ambient temperature for 24 h. The mixture was diluted with water (2.1 L) and the organic phase separated. The organic phase was washed with water (2.1 L), 2.1 L of brine, dried over magnesium sulfate, filtered over Celite and concentrated in vacuo affording a light orange oil which had a silt in the slurry. The mixture was diluted with ˜500 mL of heptane and filtered using an M filter. The precipitate (SM) was washed with 250 mL of heptane. The filtrate was concentrated in vacuo affording a thick orange oil which was seeded with solid from a previous experiment and crystallized on standing, affording a light orange hard solid. tert-butyl N-tert-butoxycarbonyl-N-(4,6-dichloropyrimidin-2-yl)carbamate (645 g, 97%). ¹H NMR (400 MHz, DMSO-d6) δ 8.07 (s, 1H), 1.44 (s, 18H). ESI-MS m/z calc. 363.07526, found 364.1 (M+1)⁺; Retention time: 2.12 minutes (LC method A).

### Step 2: tert-Butyl N-tert-butoxycarbonyl-N-[4-chloro-6-(2,6-dimethylphenyl)pyrimidin-2-yl]carbamate

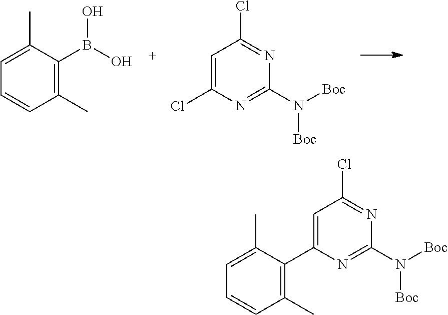 [SMILES: Cc1cccc(C)c1B(O)O ; CC(C)(C)OC(=O)N(C(=O)OC(C)(C)C)c1nc(Cl)cc(Cl)n1 ; Cc1cccc(C)c1-c1cc(Cl)nc(N(C(=O)OC(C)(C)C)C(=O)OC(C)(C)C)n1]

All solvents were degassed prior to use. To a slurry of tert-butyl N-tert-butoxycarbonyl-N-(4,6-dichloropyrimidin-2-yl)carbamate (88 g, 241.6 mmol), (2,6-dimethylphenyl)boronic acid (approximately 36.24 g, 241.6 mmol) and Cs₂CO₃ (approximately 196.8 g, 604.0 mmol) in DME (704 mL) and water (176 mL) were added. Pd(dppf)Cl₂ (approximately 8.839 g, 12.08 mmol) was added and the mixture was vigorously stirred under nitrogen at 80° C. (reflux) for 1 h (no SM remained). The reaction was cooled to ambient temperature and diluted with water (704 mL). The aqueous phase was separated and extracted with EtOAc (704 mL). The organic phase was washed with 700 mL of brine, dried over magnesium sulfate, filtered and concentrated in vacuo. The crude product was chromatographed on a 1500 g silica gel column eluting with 0-30% EtOAc/hexanes. The product fractions (eluted at 15% EtOAc) were combined and concentrated in vacuo affording the product as a clear oil which crystallized on standing. tert-butyl N-tert-butoxycarbonyl-N-[4-chloro-6-(2,6-dimethylphenyl)pyrimidin-2-yl]carbamate (81.3 g, 78%). ¹H NMR (400 MHz, DMSO-d6) δ 7.88 (s, 1H), 7.30 (dd, J=8.2, 7.0 Hz, 1H), 7.21-7.16 (m, 2H), 2.03 (s, 6H), 1.38 (s, 18H). ESI-MS m/z calc. 433.17682, found 434.1 (M+1)⁺; Retention time: 2.32 minutes (LC method A).

### Step 3: 4-Chloro-6-(2,6-dimethylphenyl)pyrimidin-2-amine (hydrochloride salt)

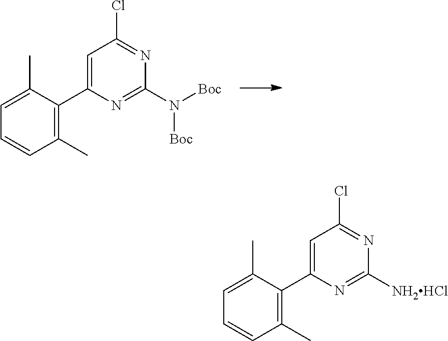

tert-Butyl N-tert-butoxycarbonyl-N-[4-chloro-6-(2,6-dimethylphenyl) pyrimidin-2-yl]carbamate (514.8 g, 915.9 mmol) was dissolved in dichloromethane (4 L). Hydrogen chloride in p-dioxane (1 L, 4 mol) was added and the mixture was stirred overnight at room temperature. The resulting precipitate was collected by vacuum filtration and dried in vacuo to obtain 4-chloro-6-(2,6-dimethylphenyl)pyrimidin-2-amine hydrochloride as a white solid (213.5 g, 82%). ¹H NMR (250 MHz, DMSO-d6) δ 7.45-6.91 (m, 3H), 6.73 (s, 1H), 2.08 (s, 6H). ESI-MS m/z calc. 233.072, found 234.1 (M+1)⁺; Retention time: 2.1 minutes (LC Method C).

### Step 4: 4-Chloro-6-(2,6-dimethylphenyl)pyrimidin-2-amine

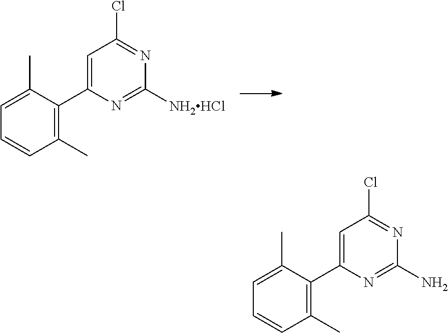

4-Chloro-6-(2,6-dimethylphenyl)pyrimidin-2-amine (hydrochloride salt) (166 g, 614.5 mmol) and 4-chloro-6-(2,6-dimethylphenyl)pyrimidin-2-amine (hydrochloride salt) (30 g, 111.0 mmol) were suspended in DCM (2.5 L), treated with NaOH (725 mL of 1 M, 725.0 mmol) and stirred at room temperature for 1 hour. The mixture was transferred into a separatory funnel and left standing over night. The DCM phase was separated and the aqueous phase with insoluble material was extracted twice more with DCM (2×500 mL). The combined brown DCM phases were stirred over magnesium sulfate and charcoal for 1 hour, filtered and the yellow solution concentrated to a volume of ˜500 mL. The solution was diluted with heptane (750 mL) and DCM was removed under reduced pressure at 60° C. to give a cream suspension. It was stirred at room temperature for 1 hour, filtered, washed with cold heptane and dried to give 4-chloro-6-(2,6-dimethylphenyl)pyrimidin-2-amine (157 g, 91%) as a cream solid. H NMR (400 MHz, DMSO-d6) δ 7.28-7.14 (m, 3H), 7.10 (d, J=7.5 Hz, 2H), 6.63 (s, 1H), 2.06 (s, 6H). ESI-MS m/z calc. 233.07198, found 234.0 (M+1)⁺; Retention time: 1.45 minutes (LC method A).

### Example B: Preparation of 4,6-dichloro-5-ethyl-pyrimidin-2-amine

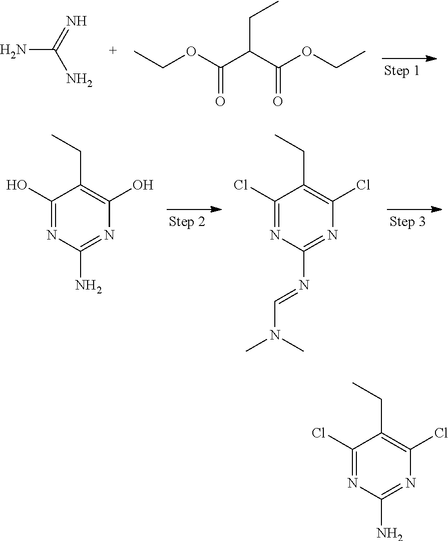

### Step 1: 2-Amino-5-ethyl-pyrimidine-4,6-diol

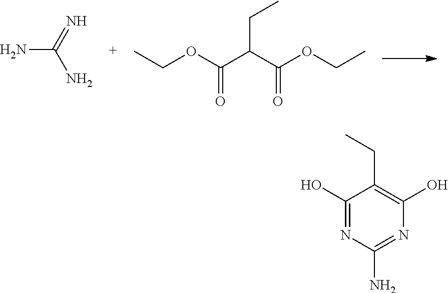

To a 2.0 L three neck flask was added ethanol (800 mL) and solid chunks of sodium metal (23 g, 1.000 mol) were added gradually and carefully under nitrogen flow and the mixture was stirred until completely dissolved. Once cooled back to room temperature guanidine (hydrochloride salt) (36.5 g, 382.1 mmol) and diethyl 2-ethylpropanedioate (56.22 g, 56 mL, 298.7 mmol) were successively added and the reaction was stirred with a mantle set at 80° C. (probe in the solution) for 17 hours. Once cooled to room temperature, the crude mixture was concentrated under reduced pressure to remove most of the ethanol. Water (400 mL) was added, the resulting solution was cooled in an ice bath and acidified to pH of 1-2 using concentrated HCl. The solids were filtered and washed with water (2×100 mL) followed by cold acetone (250 mL). The solid was dried in the fume hood overnight and transferred to a crystallization dish under high vacuum for three days to provide 2-amino-5-ethyl-pyrimidine-4,6-diol (H₂O)₂ (54.8 g, 96%) as a white solid. ESI-MS m/z calc. 155.0695, found 156.2 (M+1)+; Retention time: 0.369 minutes. ¹H NMR (300 MHz, DMSO-d6) δ 0.87 (t, J=7.2 Hz, 3H), 2.03-2.23 (m, 2H), 6.28 (s, 2H), 10.02-10.45 (m, 2H).

Step 2: N′-(4,6-Dichloro-5-ethyl-pyrimidin-2-yl)-N,N-dimethyl-formamidine

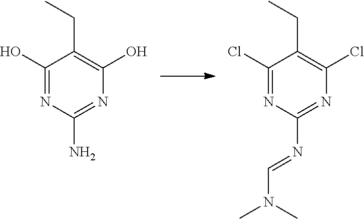

Oxalyl chloride (290.0 g, 200 mL, 2.285 mol) was slowly added to a solution of dimethylformamide (166.4 g, 177 mL, 2.277 mol) in chloroform (1.6 L) and the solution was stirred at room temperature for 30 minutes. 2-Amino-5-ethyl-pyrimidine-4,6-diol (H₂O)₂ (43.55 g, 227.8 mmol) was added then the reaction mixture was heated at 60° C. overnight. Once cooled to room temperature, the reaction mixture was diluted with saturated sodium bicarbonate solution (2.0 L) and stirred vigorously for 15 minutes. A 25% sodium hydroxide solution (250 mL) was added to reach pH ˜8-9. Layers were separated and aqueous layer was extracted with dichloromethane (2×700 mL). The organic layers were combined, dried over sodium sulfate and concentrated under reduced pressure to afford N′-(4,6-dichloro-5-ethyl-pyrimidin-2-yl)-N,N-dimethyl-formamidine (192 g, 341%) as brown oil. The crude material was used for the next step without any further purification. ESI-MS m/z calc. 246.0439, found 247.1 (M+1)+; Retention time: 1.25 minutes.

### Step 3: 4,6-Dichloro-5-ethyl-pyrimidin-2-amine

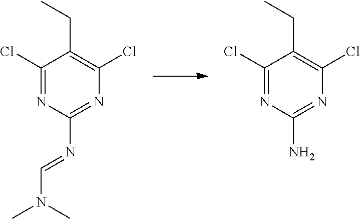

Concentrated hydrochloric acid (117 mL of 12 M, 1.404 mol) was added to a solution of N′-(4,6-dichloro-5-ethyl-pyrimidin-2-yl)-N,N-dimethyl-formamidine (57.9 g, 234.3 mmol) and the mixture was stirred at 50° C. for 150 minutes. The mixture was cooled in the freezer overnight then the solid was filtered, rinsed with cold isopropanol (350 mL) and dried to afford 4,6-dichloro-5-ethyl-pyrimidin-2-amine (35.4 g, 77%) as an off-white solid. ESI-MS m/z calc. 191.0017, found 192.1 (M+1)+; Retention time: 2.39 minutes. ¹H NMR (300 MHz, CDCl₃) ppm 1.15 (t, J=7.5 Hz, 3H), 2.75 (q, J=7.3 Hz, 2H), 5.30 (br. s., 2H).

### Example C: Preparation of 3-[[4-chloro-6-(2,6-dimethylphenyl)-5-methyl-pyrimidin-2-yl]sulfamoyl]benzoic acid

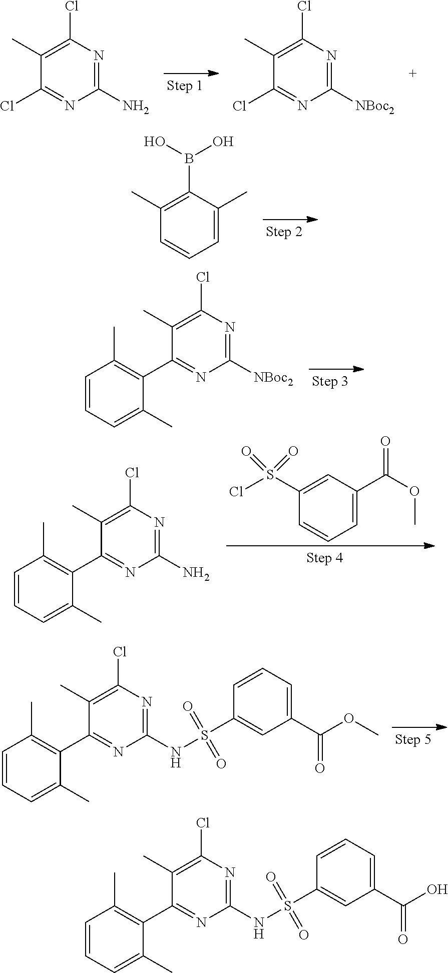

### Step 1: tert-Butyl N-tert-butoxycarbonyl-N-(4,6-dichloro-5-methyl-pyrimidin-2-yl)carbamate

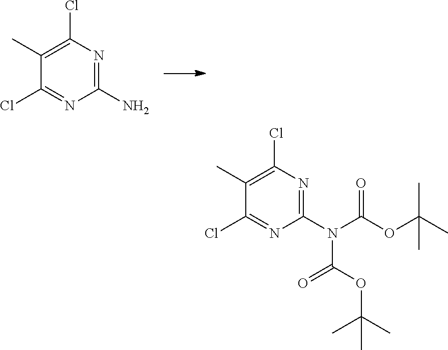

To a solution of 4,6-dichloro-5-methyl-pyrimidin-2-amine (57.85 g, 318.47 mmol) in DCM (580 mL) was added tert-butoxycarbonyl tert-butyl carbonate (159.92 g, 168.34 mL, 710.77 mmol) and DMAP (3.96 g, 32.090 mmol) at RT. The reaction was stirred for 3 h. The reaction mixture was quenched with DI H₂O (250 mL). DCM (100 mL) was added. The layers were separated, and the aqueous layer was extracted with DCM (2×250 mL). The combined organic layers were washed with aqueous saturated NaCl (250 mL), dried over anhydrous sodium sulfate, filtered and concentrated under vacuum. Crude Mass=125.71 g (Yellow Solid). The yellow solid was triturated with hexanes (300 mL, 3 h), filtered through a Type “M” Glass filter by vacuum and the solids were rinsed with Hexanes (2×200 mL). Final Product (101.00 g) was obtained as a yellow solid. tert-butyl N-tert-butoxycarbonyl-N-(4,6-dichloro-5-methyl-pyrimidin-2-yl)carbamate (101.00 g, 80%). ¹H NMR (500 MHz, Chloroform-d) δ 2.48 (s, 3H), 1.47 (s, 18H). ESI-MS m/z calc. 377.0909, found 378.0 (M+1)⁺; Retention time: 3.39 minutes; LC method T.

### Step 2: tert-Butyl N-tert-butoxycarbonyl-N-[4-chloro-6-(2,6-dimethylphenyl)-5-methyl-pyrimidin-2-yl]carbamate

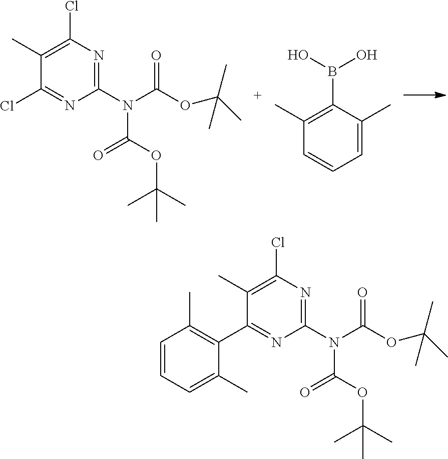

To a solution of tert-butyl N-tert-butoxycarbonyl-N-(4,6-dichloro-5-methyl-pyrimidin-2-yl)carbamate (120.85 g, 319.50 mmol) dissolved in DME (850 mL) and water (120 mL) was added (2,6-dimethylphenyl)boronic acid (57.5 g, 383.38 mmol) and cesium carbonate (271 g, 831.75 mmol) at room temperature. The solution was stirred for 10 minutes while being bubbled with a nitrogen stream. Then Pd(dppf)Cl₂ (11.7 g, 15.990 mmol) was added to the solution and heated to 80° C. overnight. The solution was cooled to room temperature before being diluted with water (500 mL) and extracted with ethyl acetate (2×1 L). The combined organic layer was washed with brine (1 L) and dried over sodium sulfate before being concentrated in vacuum. The organic residue was filtered through a pad of silica gel and washed with a solution of 1:3 ethyl acetate-hexanes (3×1 L) to give tert-butyl N-tert-butoxycarbonyl-N-[4-chloro-6-(2,6-dimethylphenyl)-5-methyl-pyrimidin-2-yl]carbamate (100.71 g, 58%). ESI-MS m/z calc. 447.19247, found 448.1 (M+1)+; Retention time: 4.24 minutes; LC method T.

### Step 3: 4-Chloro-6-(2,6-dimethylphenyl)-5-methyl-pyrimidin-2-amine

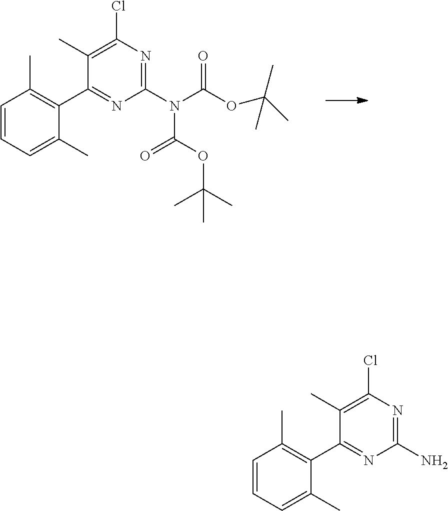 [SMILES: Cc1cccc(C)c1-c1nc(N(C(=O)OC(C)(C)C)C(=O)OC(C)(C)C)nc(Cl)c1C ; Cc1cccc(C)c1-c1nc(N)nc(Cl)c1C]

To a solution of tert-butyl N-tert-butoxycarbonyl-N-[4-chloro-6-(2,6-dimethylphenyl)-5-methyl-pyrimidin-2-yl]carbamate (100.71 g, 224.82 mmol) in DCM (500 mL) was added HCl (200 mL of 4 M, 800.00 mmol) in dioxane. The solution was stirred at room temperature overnight before being concentrated in vacuum. The residue was then basified with sodium bicarbonate (500 mL) and extracted with ethyl acetate (1 L). The organic layer was washed with brine (400 mL) and dried over sodium sulfate. The organic phase was concentrated then the residue was triturated with hexanes (2×200 mL) to give 4-chloro-6-(2,6-dimethylphenyl)-5-methyl-pyrimidin-2-amine (54.88 g, 99%) as an off-white solid. ESI-MS m/z calc. 247.08763, found 248.2 (M+1)+; Retention time: 2.94 minutes; LC method T.

### Step 4: Methyl 3-[[4-chloro-6-(2,6-dimethylphenyl)-5-methyl-pyrimidin-2-yl]sulfamoyl]benzoate

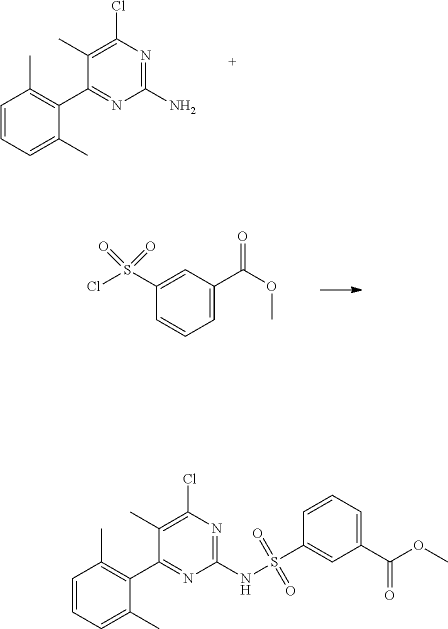 [SMILES: Cc1cccc(C)c1-c1nc(N)nc(Cl)c1C ; COC(=O)c1cccc(S(=O)(=O)Cl)c1 ; COC(=O)c1cccc(S(=O)(=O)Nc2nc(Cl)c(C)c(-c3c(C)cccc3C)n2)c1]

To a solution of 4-chloro-6-(2,6-dimethylphenyl)-5-methyl-pyrimidin-2-amine (35 g, 141.29 mmol) in THF (400 mL) at 0° C. was added methyl 3-chlorosulfonylbenzoate (50 g, 213.08 mmol). Then Lithium tert-amoxide (46.428 g, 159 mL of 40% w/w, 197.40 mmol) was added to the solution dropwise keeping the temperature below 5° C. The solution was allowed to warm to room temperature while it stirred for 3 hours. The solution was acidified with 1M HCl (200 mL) and extracted with ethyl acetate (3×200 mL). The organic layer was washed with brine (300 mL) and dried over sodium sulfate. The organic layer was then concentrated in vacuum to give methyl 3-[[4-chloro-6-(2,6-dimethylphenyl)-5-methyl-pyrimidin-2-yl]sulfamoyl]benzoate (63.01 g, 100%) as a yellow solid. ESI-MS m/z calc. 445.0863, found 446.2 (M+1)+; Retention time: 3.63 minutes; LC method T.

### Step 5: 3-[[4-Chloro-6-(2,6-dimethylphenyl)-5-methyl-pyrimidin-2-yl]sulfamoyl]benzoic acid

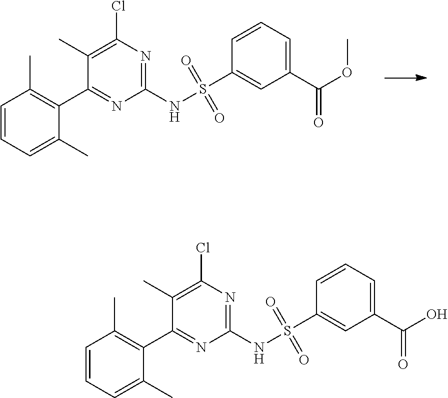 [SMILES: COC(=O)c1cccc(S(=O)(=O)Nc2nc(Cl)c(C)c(-c3c(C)cccc3C)n2)c1 ; Cc1cccc(C)c1-c1nc(NS(=O)(=O)c2cccc(C(=O)O)c2)nc(Cl)c1C]

To a solution of methyl 3-[[4-chloro-6-(2,6-dimethylphenyl)-5-methyl-pyrimidin-2-yl]sulfamoyl]benzoate (59.51 g, 133.45 mmol) in THF (500 mL) was added an aqueous solution of NaOH (300 mL of 2 M, 600.00 mmol) and the mixture was stirred for 2 hours at room temperature. The solution was acidified using 3M HCl (500 mL) and extracted with ethyl acetate (2×500 mL) before being washed with brine (500 mL). The organic layer was dried over sodium sulfate and concentrated in vacuum. The organic residue was then recrystallized with ethanol and filtered to give 3-[[4-chloro-6-(2,6-dimethylphenyl)-5-methyl-pyrimidin-2-yl]sulfamoyl]benzoic acid (34.44 g, 56%) as a white solid. ¹H NMR (500 MHz, DMSO-d6) δ 8.43 (t, J=1.8 Hz, 1H), 8.18 (dt, J=7.8, 1.4 Hz, 1H), 8.10 (ddd, J=7.9, 2.0, 1.2 Hz, 1H), 7.66 (t, J=7.8 Hz, 1H), 7.25 (t, J=7.6 Hz, 1H), 7.12 (d, J=7.6 Hz, 2H), 1.85 (s, 3H), 1.74 (s, 6H). ESI-MS m/z calc. 431.07065, found 432.4 (M+1)+; Retention time: 2.43 minutes; LC method T.

### Example D: Preparation of 3-[[5-tert-butyl-4-chloro-6-(o-tolyl)pyrimidin-2-yl]sulfamoyl]benzoic acid

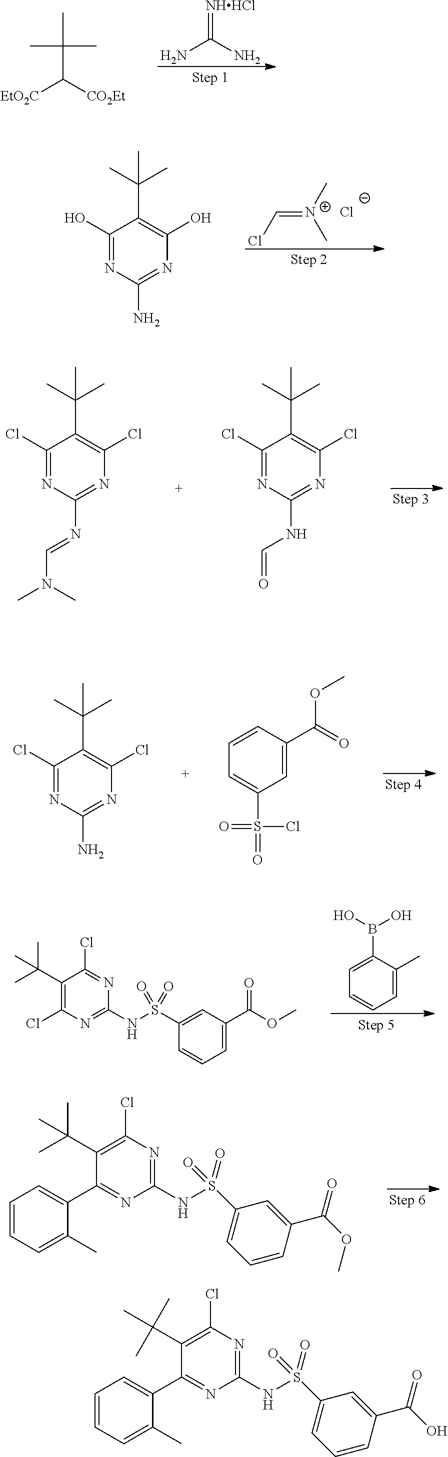

### Step 1: 2-Amino-5-tert-butyl-pyrimidine-4,6-diol

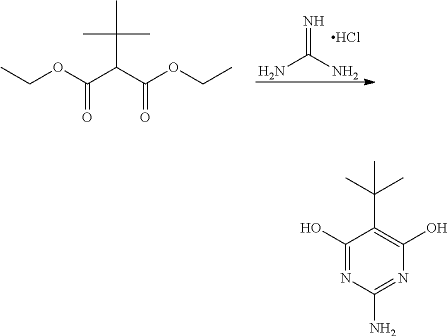

To a 2.0 L flask charged with ethanol (760 mL), solid chunks of sodium metal (17.7 g, 769.9 mmol) were added gradually and carefully and the mixture was stirred until all solids completely dissolved. Once cooled back to room temperature guanidine (hydrochloride salt) (28.3 g, 296.2 mmol) and diethyl 2-tert-butylpropanedioate (50 g, 231.2 mmol) were successively added and the reaction was heated at 80° C. for 17 hours. Once cooled to room temperature, the crude mixture was concentrated under reduced pressure to remove most of the ethanol. Water was added to complete dissolution (350 mL), the resulting solution was cooled in an ice bath and acidified to pH of 1-2 using concentrated HCl. The solids were filtered and washed with water (2×100 mL) then with acetone (2×100 mL) and dried under high vacuum to provide 2-amino-5-tert-butyl-pyrimidine-4,6-diol hydrate (43.4 g, 93%) as an off-white solid. ¹H NMR (300 MHz, DMSO-d₆) δ ppm 1.01 (s, 9H). ESI-MS m/z calc. 183.10078, found 184.2 (M+1)⁺; Retention timed 0.71 minutes; LC method C.

### Step 2: N′-(5-tert-Butyl-4,6-dichloro-pyrimidin-2-yl)-N,N-dimethyl-formamidine and N-(5-tert-butyl-4,6-dichloro-pyrimidin-2-yl)formamide

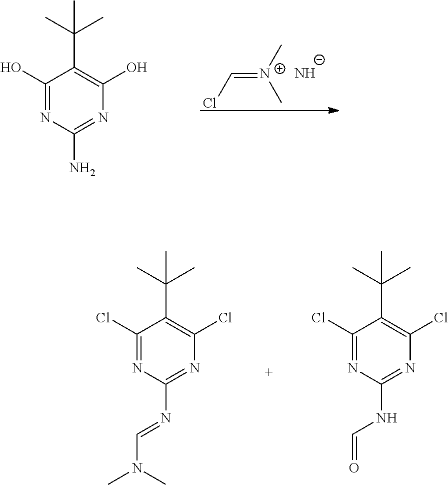

2-Amino-5-tert-butyl-pyrimidine-4,6-diol (2 g, 10.92 mmol) was dissolved in chloroform (40 mL) and (Chloromethylene)dimethyliminium chloride (11.3 g, 88.28 mmol) was added and the mixture was heated at reflux for 2 hours under nitrogen atmosphere then left to cool down and stirred overnight at room temperature. It was heated to reflux for another 4 hours. The reaction mixture was cooled to room temperature then partitioned between ice cold saturated sodium bicarbonate (50 mL) and dichloromethane (2×50 mL). The combined organics were dried over sodium sulfate then filtered and concentrated under reduced pressure. The resulting residue was purified on silica gel using 20 then 45% ethyl acetate in heptane to give mainly two products as a white solid N′-(5-tert-butyl-4,6-dichloro-pyrimidin-2-yl)-N,N-dimethyl-formamidine (1.2 g, 40%) ESI-MS m/z calc. 275.178, found 275.1 (M)⁺; Retention time: 1.568 minutes (LC method C), ¹H NMR (300 MHz, CDCl₃) δ ppm 1.57-1.63 (s, 9H), 3.15 (d, J=2.1 Hz, 6H), 8.59 (s, 1H) and N-(5-tert-butyl-4,6-dichloro-pyrimidin-2-yl)formamide (600 mg, 22%), ESI-MS m/z calc. 248.109, found 248.1 (M)⁺; Retention time: 2.05 minutes (LC method C), ¹H NMR (300 MHz, CDCl₃) δ ppm 1.65 (s, 9H), 7.83 (d, J=8.8 Hz, 1H), 9.35 (d, J=10.6 Hz, 1H).

### Step 3: 5-tert-Butyl-4,6-dichloro-pyrimidin-2-amine

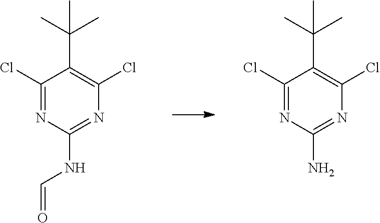

To N-(5-tert-butyl-4,6-dichloro-pyrimidin-2-yl)formamide (330 mg, 1.330 mmol) dissolved in isopropanol (7 mL) was added HCl (0.6 mL of 12 M, 7.200 mmol) and the mixture was stirred 30 minutes at 50° C. The reaction mixture was concentrated under reduced pressure to provide the desired product 5-tert-butyl-4,6-dichloro-pyrimidin-2-amine (277 mg, 95%) as a white solid. ¹H NMR (300 MHz, CDCl₃) δ ppm 1.61 (s, 9H), 5.07 (br. s., 2H). ESI-MS m/z calc. 219.033, found 220.1 (M+1)⁺; Retention time: 2.048 minutes; LC method C.

### Step 4: Methyl 3-[(5-tert-butyl-4,6-dichloro-pyrimidin-2-yl)sulfamoyl]benzoate

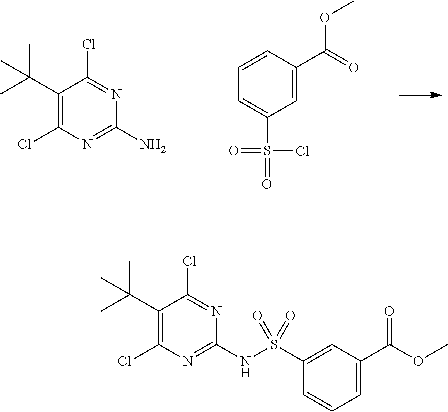 [SMILES: CC(C)(C)c1c(Cl)nc(N)nc1Cl ; COC(=O)c1cccc(S(=O)(=O)Cl)c1 ; COC(=O)c1cccc(S(=O)(=O)Nc2nc(Cl)c(C(C)(C)C)c(Cl)n2)c1]

Sodium hydride (approximately 6.359 g, 7.066 mL, 159.0 mmol) was suspended in NMP (35.00 mL). At 0° C., a solution of 5-tert-butyl-4,6-dichloro-pyrimidin-2-amine (7.00 g, 31.80 mmol) in NMP (14.00 mL) was slowly added dropwise. After stirring at room temperature for 30 minutes, it was again cooled to 0° C. before the slow dropwise addition of methyl 3-chlorosulfonylbenzoate (approximately 9.701 g, 41.34 mmol) in NMP (28.00 mL). The reaction mixture was stirred at 0° C. for 1 hour. The reaction mixture was cooled to −10° C., and 1 N HCl was very slowly and carefully added. When bubbling stopped, the mixture was diluted with 100 mL 1 N HCl and 50 mL water. It was extracted with EtOAc (2×100 mL). The organic layers were combined and washed with brine (1×100 mL), dried over sodium sulfate, filtered and concentrated under reduced pressure. The crude product was purified by chromatography on a 40 gram silica gel column eluting with a 0-40% EtOAc/hexane gradient over 40 minutes to give methyl 3-[(5-tert-butyl-4,6-dichloro-pyrimidin-2-yl)sulfamoyl]benzoate (6.51 g, 49%). ESI-MS m/z calc. 417.03168, found 418.1 (M+1)⁺; Retention time: 1.95 minutes; LC method A.

### Step 5: Methyl 3-{[5-tert-butyl-4-chloro-6-(2-methylphenyl)pyrimidin-2-yl]sulfamoyl}benzoate

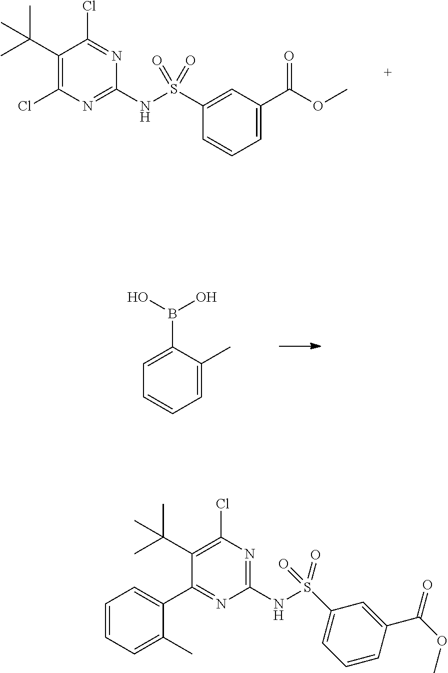

Methyl 3-[(5-tert-butyl-4,6-dichloro-pyrimidin-2-yl)sulfamoyl]benzoate (1 g, 2.391 mmol), o-tolylboronic acid (195 mg, 1.434 mmol), potassium carbonate (1.09 g, 7.887 mmol), and trans-dichlorobis(triphenylphosphine)palladium(II) (60 mg, 0.08548 mmol) were dissolved in dioxane (10 mL) and water (2 mL) in a nitrogen purged vial. The reaction mixture was then heated to 90° C. for two hours. At this point, the reaction mixture was cooled to room temperature and poured into a flask containing 1 M HCl, and ethyl acetate. The aqueous and organics were separated, and the aqueous was extracted 2×ethyl acetate. The combined organics were washed with brine, dried over sodium sulfate and concentrated. The resulting crude was subjected to chromatography on silica gel with a gradient of 0-60% ethyl acetate in hexanes, giving mostly overlapping fractions containing desired product and starting material. The mixed fractions containing the desired mono-arylated product were subjected to chromatography on silica gel a second time using a gradient of 0-50% ethyl acetate in dichloromethane. The fractions containing a majority of the monoarylated product were combined and concentrated to give a white solid: Methyl 3-{[5-tert-butyl-4-chloro-6-(2-methylphenyl)pyrimidin-2-yl]sulfamoyl}benzoate (175 mg, 15%). ESI-MS m/z calc. 473.1176, found 474.3 (M+1)⁺; Retention time: 0.82 minutes; LC method D.

### Step 6: 3-[[5-tert-Butyl-4-chloro-6-(o-tolyl)pyrimidin-2-yl]sulfamoyl]benzoic acid

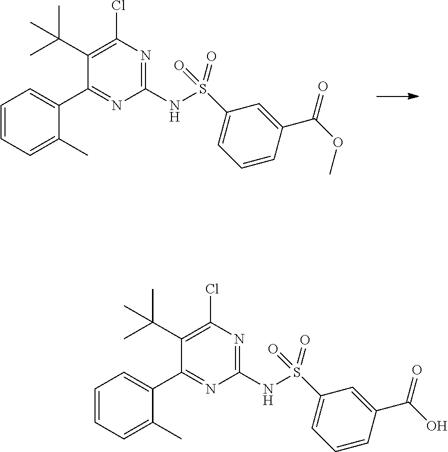 [SMILES: COC(=O)c1cccc(S(=O)(=O)Nc2nc(Cl)c(C(C)(C)C)c(-c3ccccc3C)n2)c1 ; Cc1ccccc1-c1nc(NS(=O)(=O)c2cccc(C(=O)O)c2)nc(Cl)c1C(C)(C)C]

Methyl 3-{[5-tert-butyl-4-chloro-6-(2-methylphenyl)pyrimidin-2-yl]sulfamoyl}benzoate (175 mg, 0.3692 mmol) was dissolved in THF (923.0 μL) and isopropanol (307.7 μL), cooled to 0° C., and sodium hydroxide (approximately 1.477 mL of 1 M, 1.477 mmol) (1M aqueous) was added. After 3 hours at 0° C. the reaction mixture was acidified with 1 M HCl, poured into 0.5 M HCl, and extracted 3× with ethyl acetate. The combined organics were washed with water, brine, and dried over sodium sulfate. Concentration afforded 3-[[5-tert-butyl-4-chloro-6-(o-tolyl)pyrimidin-2-yl]sulfamoyl]benzoic acid (164 mg, 97%) as a white solid which was used without further purification. ESI-MS m/z calc. 459.10196, found 460.3 (M+1)⁺; Retention time: 0.75 minutes; LC method D.

### Example E: Preparation of 3-[[4-chloro-6-(o-tolyl)-5-(trifluoromethyl)pyrimidin-2-yl]sulfamoyl]benzoic acid

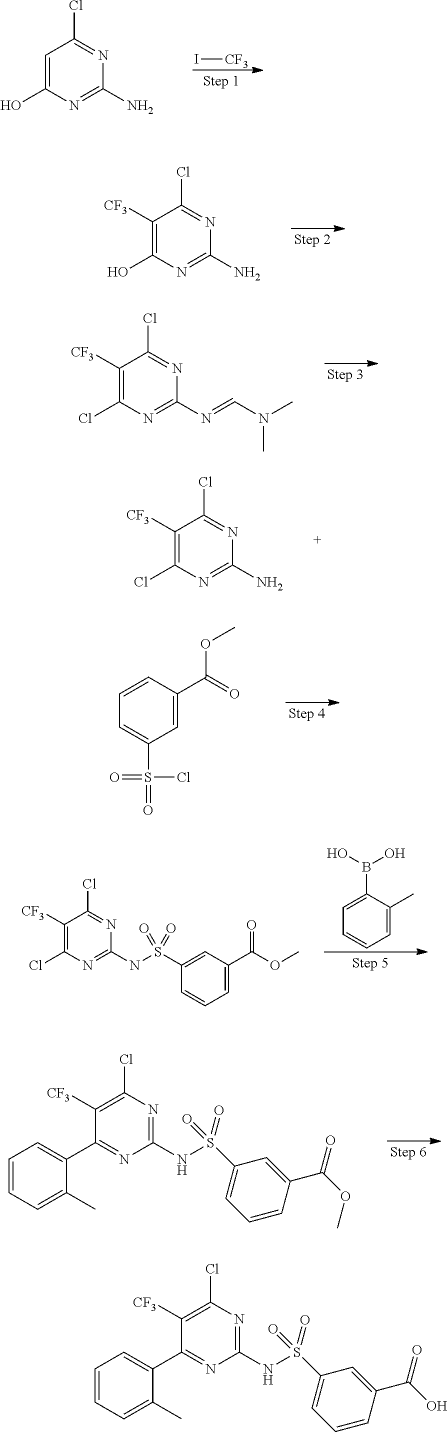

### Step 1: 2-Amino-6-chloro-5-(trifluoromethyl)pyrimidin-4-ol

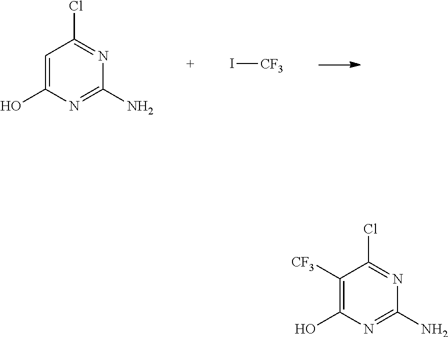 [SMILES: Nc1nc(O)cc(Cl)n1 ; FC(F)(F)I ; Nc1nc(O)c(C(F)(F)F)c(Cl)n1]

An empty 1.0 L flask was cooled in a dry ice bath and trifluoro(iodo)methane (121.15 g, 618.4 mmol) was condensed inside. The flask was then removed from the cold bath and DMSO (200 mL) was added slowly along the inner sides of the flask to give a slurry. This slurry was slowly warmed up until a clear solution was obtained. This solution was then added to a three-neck 1.0 L flask containing a solution of 2-amino-6-chloro-pyrimidin-4-ol (32.20 g, 221.2 mmol) in DMSO (400 mL) at room temperature. Then, a solution of ferrous sulfate was added slowly (66 mL of a roughly 1 M aqueous solution, prepared by dissolving 27.8 g of ferrous sulfate heptahydrate in 100 mL of water, 50 mmol) and the temperature, monitored with a probe inside the reaction, rose a few degrees. To this solution was then added dropwise and carefully, aqueous hydrogen peroxide (38 mL of a 35% aqueous solution, 391 mmol) at such a rate that at the end of the addition, the internal temperature was at 41° C. (over 4 hours). After another 30 minutes, the crude mixture was added equally (about 400 mL each) to 2 separate 5.0 L flask containing water (1.5 L) and ice cubes (1.5 L, used to cool the water instead of an external bath in addition to having more water in the flask). Upon stirring, solids crashed out and there was also a foam at the top. This crude mixture was filtered and the solids were washed with water, then dried under high vacuum for about 2-3 days to afford 2-amino-6-chloro-5-(trifluoromethyl)pyrimidin-4-ol (34.86 g, 74%) as an off-white solid. ESI-MS m/z calc. 212.9917, found 214.1 (M+1)⁺; Retention time: 1.33 minutes (LC method C).

### Step 2: N′-[4,6-Dichloro-5-(trifluoromethyl)pyrimidin-2-yl]-N,N-dimethyl-formamidine

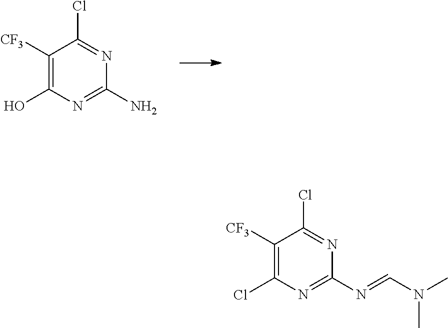 [SMILES: Nc1nc(O)c(C(F)(F)F)c(Cl)n1 ; CN(C)/C=N/c1nc(Cl)c(C(F)(F)F)c(Cl)n1]

Oxalyl chloride (124.70 g, 86 mL, 982.46 mmol) was slowly added to a solution of dimethylformamide (71.440 g, 76 mL, 977.37 mmol) in chloroform (1.25 L) and the solution was stirred at room temperature for 30 minutes. 2-Amino-6-chloro-5-(trifluoromethyl)pyrimidin-4-ol (34.86 g, 163.24 mmol) was added then the reaction mixture was heated at 60° C. for 2 h. Once cooled to room temperature, the reaction mixture was diluted with saturated sodium bicarbonate solution (2 L) and stirred vigorously for 15 minutes. A 25% sodium hydroxide solution (180 mL) was added to reach pH ˜8-9. The layers were separated, and the aqueous layer was extracted with dichloromethane (2×500 mL). The organic layers were combined, dried over sodium sulfate and concentrated under reduced pressure to afford N′-[4,6-dichloro-5-(trifluoromethyl)pyrimidin-2-yl]-N,N-dimethyl-formamidine (126.7 g, 270%) as a brown oil. ESI-MS m/z calc. 286, found 287.1 (M+1)⁺; Retention time: 1.82 minutes (LC method C).

### Step 3: 4,6-Dichloro-5-(trifluoromethyl)pyrimidin-2-amine

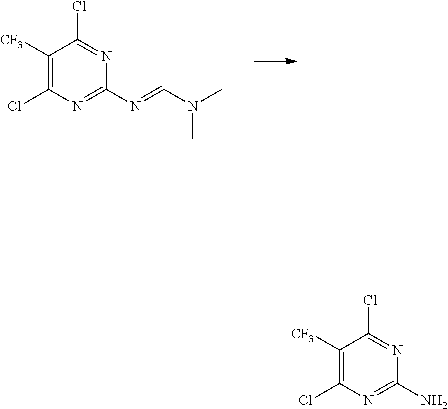 [SMILES: CN(C)/C=N/c1nc(Cl)c(C(F)(F)F)c(Cl)n1 ; Nc1nc(Cl)c(C(F)(F)F)c(Cl)n1]

Hydrochloric acid (85 mL of 12 M, 1.0200 mol) was added to a solution of N-[4,6-dichloro-5-(trifluoromethyl)pyrimidin-2-yl]-N,N-dimethyl-formamidine (46.86 g, 163.24 mmol) in isopropanol (950 mL) and the mixture was stirred at 50° C. for 90 minutes. The solution was concentrated under reduced pressure and ethyl acetate (800 mL) was added. The organic phase was washed with water (2×300 mL) and brine (300 mL), dried over anhydrous sodium sulfate and concentrated under reduced pressure. The residue was purified on silica gel chromatography (2 successive columns) eluting from 0 to 40% ethyl acetate in heptanes then triturated in a mix of ethyl acetate and heptanes (˜1:19), filtered and dried to afford 4,6-dichloro-5-(trifluoromethyl)pyrimidin-2-amine (24.4 g, 63%) as a white solid. ¹H NMR (300 MHz, DMSO-d₆) δ 8.30 (br. s., 2H). ¹⁹F NMR (282 MHz, DMSO-d₆) δ −53.4 (s, 3F). ESI-MS m/z calc. 230.9578, found 232.0 (M+1)⁺; Retention time: 2.51 minutes (LC method B).

### Step 4: Methyl 3-[[4,6-dichloro-5-(trifluoromethyl)pyrimidin-2-yl]sulfamoyl]benzoate

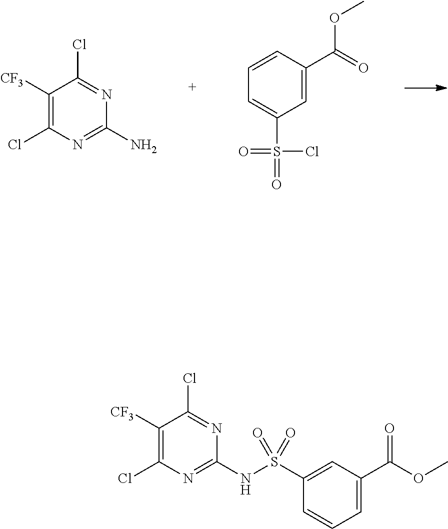 [SMILES: Nc1nc(Cl)c(C(F)(F)F)c(Cl)n1 ; COC(=O)c1cccc(S(=O)(=O)Cl)c1 ; COC(=O)c1cccc(S(=O)(=O)Nc2nc(Cl)c(C(F)(F)F)c(Cl)n2)c1]

Sodium hydride (approximately 6.031 g, 6.701 mL, 150.8 mmol) was suspended in NMP (35.00 mL). At 0° C., A solution of 4,6-dichloro-5-(trifluoromethyl)pyrimidin-2-amine (7.00 g, 30.17 mmol) in NMP (14.00 mL) was slowly added dropwise. After stirring at room temperature for 30 minutes, it was again cooled to 0° C. before the slow dropwise addition of methyl 3-chlorosulfonylbenzoate (approximately 9.203 g, 39.22 mmol) in NMP (28.00 mL). The reaction mixture was stirred at 0° C. for 1 hour. The reaction mixture was cooled to −10° C., and 1 N HCl was very slowly and carefully added. When bubbling stopped, the mixture was diluted with 100 mL 1 N HCl and 50 mL water. It was extracted with EtOAc (2×100 mL). The organic layers were combined and washed with brine (1×100 mL), dried over sodium sulfate, filtered and concentrated under reduced pressure. The crude product was purified by chromatography on a 40 gram silica gel column eluting with a 0-40% EtOAc/hexane gradient over 40 minutes to give methyl 3-[[4,6-dichloro-5-(trifluoromethyl)pyrimidin-2-yl]sulfamoyl]benzoate (7.17 g, 55%). ESI-MS m/z calc. 428.95645, found 430.0 (M+1)+; Retention time: 1.76 minutes; LC method A.

### Step 5: Methyl 3-{[4,6-dichloro-5-(trifluoromethyl)pyrimidin-2-yl]sulfamoyl}benzoate

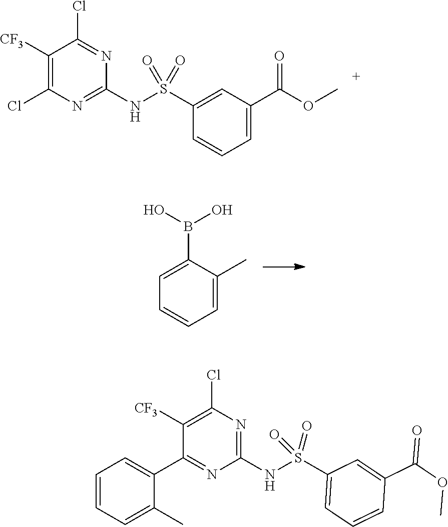

Methyl 3-[[4,6-dichloro-5-(trifluoromethyl)pyrimidin-2-yl]sulfamoyl]benzoate (1 g, 2.325 mmol), o-tolylboronic acid (190 mg, 1.398 mmol), potassium carbonate (1.06 g, 7.670 mmol), and trans-dichlorobis(triphenylphosphine)palladium(II) (60 mg, 0.08548 mmol) were dissolved dioxane (10 mL) and water (2 mL) in a nitrogen purged vial. The reaction mixture was then heated to 90° C. for two hours. At this point, the reaction mixture was cooled to room temperature and poured into a flask containing 1 M HCl, and ethyl acetate. The aqueous and organic layers were separated, and the aqueous was extracted with 2× ethyl acetate. The combined organics were washed with brine, dried over sodium sulfate and concentrated. The resulting crude was subjected to chromatography on silica gel with a gradient of 0-60% ethyl acetate in hexanes, giving mostly overlapping fractions containing desired product and starting material. The mixed fractions containing the desired product were subjected to chromatography on silica gel a second time using a gradient of 0-50% ethyl acetate in dichloromethane to give as a white solid, methyl 3-{[4,6-dichloro-5-(trifluoromethyl)pyrimidin-2-yl]sulfamoyl}benzoate (220 mg, 19%). ESI-MS m/z calc. 485.0424, found 486.2 (M+1)+; Retention time: 0.78 minutes; LC method D.

### Step 6: 3-[[4-Chloro-6-(o-tolyl)-5-(trifluoromethyl)pyrimidin-2-yl]sulfamoyl]benzoic acid

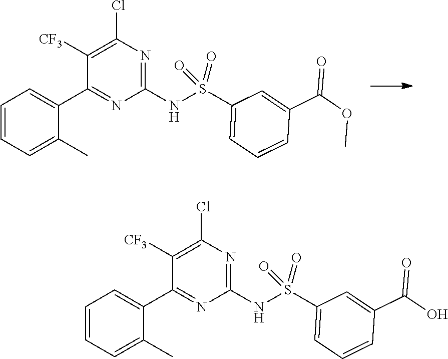 [SMILES: COC(=O)c1cccc(S(=O)(=O)Nc2nc(Cl)c(C(F)(F)F)c(-c3ccccc3C)n2)c1 ; Cc1ccccc1-c1nc(NS(=O)(=O)c2cccc(C(=O)O)c2)nc(Cl)c1C(F)(F)F]

Methyl 3-{[4,6-dichloro-5-(trifluoromethyl)pyrimidin-2-yl]sulfamoyl}benzoate (220 mg, 0.4528 mmol) was dissolved in THF (1.132 mL) and Isopropanol (377.3 μL), cooled to 0° C., and sodium hydroxide (approximately 1.811 mL of 1 M, 1.811 mmol) (1M aqueous) was added. After 3 hours at 0° C. the reaction mixture was acidified with 1 M HCl, poured into 0.5 M HCl, and extracted 3× ethyl acetate. The combined organics were washed with water, brine, and dried over sodium sulfate. Concentration afforded 3-[[4-chloro-6-(o-tolyl)-5-(trifluoromethyl)pyrimidin-2-yl]sulfamoyl]benzoic acid (206 mg, 96%) as a white solid which was used without further purification. ESI-MS m/z calc. 471.02673, found 472.3 (M+1)+; Retention time: 0.7 minutes; LC method D.

### Example F: Preparation of (2R)-2-amino-4,4-dimethyl-pentan-1-ol

### Step 1: (2R)-2-Amino-4,4-dimethyl-pentan-1-ol

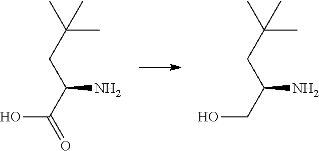

To a solution of (2R)-2-amino-4,4-dimethyl-pentanoic acid (15 g, 103.3 mmol) in THE (150 mL) at 0° C. was added borane-THF (260 mL of 1 M, 260.0 mmol) dropwise keeping the reaction temperature <10° C. The addition took approximately 30 minutes. The mixture was allowed to warm to ambient temperature and stirred for 22 hours. The reaction was quenched with the slow addition of methanol (80 mL, 1.975 mol) and the solvent was removed in vacuo. The residue was co-evaporated 3× with methanol (200 mL, 4.937 mol) The crude residue was diluted with HCl (200 mL of 1 M, 200.0 mmol) and washed with 200 mL of MTBE. The aqueous phase was evaporated to remove residual organic solvent. The water was further removed in vacuo affording an off-white solid. The solid was further dried using an acetonitrile azeotrope. The solid was slurried in 200 mL of ACN and the precipitate collected using a M frit. The solid was air dried for 1 hour, then in vacuo at 45° C. for 20 hours to give (2R)-2-amino-4,4-dimethyl-pentan-1-ol (hydrochloride salt) (14.73 g, 85%). ¹H NMR (400 MHz, DMSO-d₆) δ 7.80 (s, 3H), 5.36 (t, J=5.1 Hz, 1H), 3.59 (dt, J=11.7, 4.1 Hz, 1H), 3.42-3.34 (m, 1H), 3.10 (dq, J=7.7, 3.8 Hz, 1H), 1.46 (dd, J=14.5, 7.1 Hz, 1H), 1.33 (dd, J=14.5, 3.5 Hz, 1H), 0.91 (s, 9H). ESI-MS m/z calc. 131.13101, found 132.1 (M+1)+; Retention time: 0.51 minutes (LC method A).

### Example G: Preparation of (2R)-2-amino-5,5,5-trifluoro-4,4-dimethyl-pentan-1-ol

### Step 1: 4,4,4-Trifluoro-3,3-dimethyl-butanal

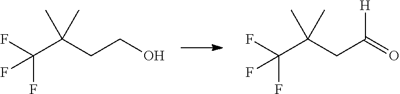

A 1 L three-neck flask was charged with 4,4,4-trifluoro-3,3-dimethyl-butan-1-ol (8.987 g, 57.555 mmol), DCM (63 mL), water (63 mL), NaBr (544 mg, 5.2870 mmol), sodium bicarbonate (12.32 g, 146.66 mmol) and TEMPO (92 mg, 0.5888 mmol). The mixture was cooled with ice-water bath. An aqueous solution of NaOCl (47 mL of 1.31 M, 61.570 mmol) was added dropwise over 2 h at 2.5-4.4° C. After the addition, the mixture was stirred for 10 minutes. The two layers was separated. The aqueous phase was extracted with DCM (2×15 mL). The combined organic layers were dried with sodium sulfate and filtered to give 113.7 g (about 80 mL) of crude product in DCM, which was used directly the next step. ¹H NMR (300 MHz, CDCl₃) δ 9.82-9.78 (m, 1H), 2.54 (d, J=2.6 Hz, 2H), 1.28 (s, 6H). ¹⁹F NMR (282 MHz, CDCl₃) δ −79.11 (s, 3F).

### Step 2: (2R)-5,5,5-Trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile

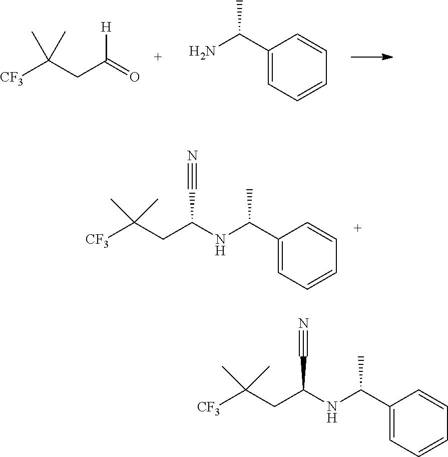

To a DCM (80 mL) solution of 4,4,4-trifluoro-3,3-dimethyl-butanal (113.7 g, 57.540 mmol) (purity about 7.8%) was added MeOH (110 mL). The mixture was cooled with ice-water bath. (1R)-1-phenylethanamine (8.46 g, 69.814 mmol) was added, followed by acetic acid (4.41 g, 73.436 mmol). The mixture was stirred at 0° C. for 10 min, then NaCN (3.56 g, 72.642 mmol) was added. The mixture was allowed to warm to rt slowly and stirred overnight. The reaction mixture was cooled to 0° C. and a solution of potassium carbonate (4 g) in water (20 mL) was added dropwise, followed by brine (40 mL). The mixture was extracted with DCM (2×100 mL). The organic layers were dried with sodium sulfate, filtered and concentrated. The residue was purified by flash chromatography (120 g silica gel, heptanes/EtOAc 0-30%) to afford a 4:1 mixture of (2R)-5,5,5-trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile (14.87 g, 91%) as a colorless oil. ESI-MS m/z calc. 284.15002, found 285.2 (M+1)⁺; Retention time: 3.38 minutes; LC method U.

### Step 3: (2R)-5,5,5-Trifluoro-4,4-dimethyl-2-[[(1R)-1-phenylethyl]amino]pentanamide and (2S)-5,5,5-trifluoro-4,4-dimethyl-2-[[(1R)-1-phenylethyl]amino]pentanamide

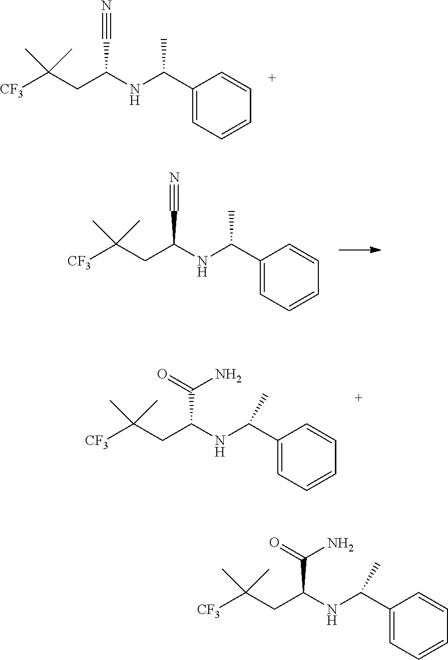 [SMILES: C[C@@H](N[C@@H](C#N)CC(C)(C)C(F)(F)F)c1ccccc1 ; C[C@@H](N[C@H](C#N)CC(C)(C)C(F)(F)F)c1ccccc1 ; C[C@@H](N[C@H](CC(C)(C)C(F)(F)F)C(N)=O)c1ccccc1 ; C[C@@H](N[C@@H](CC(C)(C)C(F)(F)F)C(N)=O)c1ccccc1]

To a solution of a 4:1 mixture of (2R)-5,5,5-trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile (14.87 g, 52.300 mmol) in DCM (105 mL) was added sulfuric acid (56.3 g, 551.06 mmol). The mixture was stirred at rt overnight, poured on crude ice (200 g) and neutralized to pH 9 with 28% NH₃ in water (100 mL). The mixture was extracted with DCM (500 mL). The organic layer was dried with sodium sulfate, filtered and concentrated. The residue was purified by flash chromatography (330 g silica gel, heptanes/EtOAc 20-50%) to afford (2R)-5,5,5-trifluoro-4,4-dimethyl-2-[[(1R)-1-phenylethyl]amino]pentanamide (10.77 g, 68%) as a white solid. ¹H NMR (300 MHz, CDCl₃) δ 7.39-7.22 (m, 5H), 6.35 (br. s., 1H), 5.55 (br. s., 1H), 3.65 (q, J=6.5 Hz, 1H), 2.93 (dd, J=7.6, 3.8 Hz, 1H), 1.87 (dd, J=15.0, 3.8 Hz, 1H), 1.65-1.56 (m, 2H), 1.35 (d, J=6.5 Hz, 3H), 1.04 (s, 3H), 1.00 (s, 3H). ¹⁹F NMR (282 MHz, CDCl₃) δ −78.77 (s, 3F). 99.4% de by ¹⁹F NMR.

### Step 4: (2R)-5,5,5-Trifluoro-4,4-dimethyl-2-[[(1R)-1-phenylethyl]amino]pentanoic acid

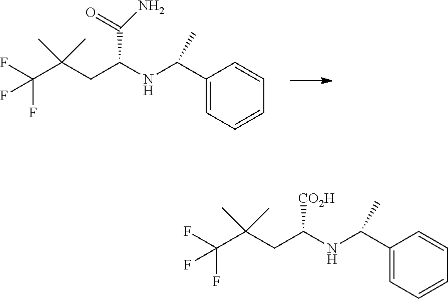 [SMILES: C[C@@H](N[C@H](CC(C)(C)C(F)(F)F)C(N)=O)c1ccccc1 ; C[C@@H](N[C@H](CC(C)(C)C(F)(F)F)C(=O)O)c1ccccc1]

To a solution of (2R)-5,5,5-trifluoro-4,4-dimethyl-2-[[(1R)-1-phenylethyl]amino]pentanamide (11.35 g, 37.541 mmol) in HOAc (50 mL) was added conc. HCl (65 mL of 11.8 M, 767.00 mmol), followed by water (50 mL). A white precipitate appeared. The mixture was heated at 100° C. for 66 hours. More conc. HCl (40 mL of 11.8 M, 472.00 mmol) and HOAc (10 mL) were added. The mixture was stirred at 100° C. overnight. More HCl in water (20 mL of 6 M, 120.00 mmol) was added. After 7 hours at 100° C., more HCl in water (20 mL of 6 M, 120.00 mmol) was added. The mixture was stirred at 100° C. overnight. It became a clear solution. More HCl in water (20 mL of 6 M, 120.00 mmol) was added. The mixture was stirred at 100° C. for 7 hours, more HCl in water (20 mL of 6 M, 120.00 mmol) was added. The mixture was stirred at 100° C. overnight. The mixture was concentrated and co-evaporated with water (50 mL). The residue (17 g) was mixed with water (25 mL) at 50° C. for 20 minutes, cooled with ice-water bath for 20 min and filtered. The crude product was mixed with 1,4-dioxane (60 mL). The mixture was concentrated and dried on vacuum overnight to give (2R)-5,5,5-trifluoro-4,4-dimethyl-2-[[(1R)-1-phenylethyl]amino]pentanoic acid (hydrochloride salt) (13.04 g, 97%) as an off-white solid. ¹H NMR (300 MHz, DMSO-d₆) δ 10.09 (br. s., 1H), 7.54-7.31 (m, 5H), 7.29-7.05 (m, 1H), 4.07 (q, J=5.9 Hz, 1H), 3.16-2.98 (m, 1H), 2.08-1.83 (m, 2H), 1.49 (d, J=6.5 Hz, 3H), 0.99 (s, 3H), 0.92 (s, 3H). ¹⁹F NMR (282 MHz, DMSO-d₆) δ −78.28 (s, 3F). ESI-MS m/z calc. 303.14462, found 304.2 (M+1)+; Retention time: 1.98 minutes; LC method U.

### Step 5: (2R)-5,5,5-Trifluoro-4,4-dimethyl-2-[[(1R)-1-phenylethyl]amino]pentan-1-ol

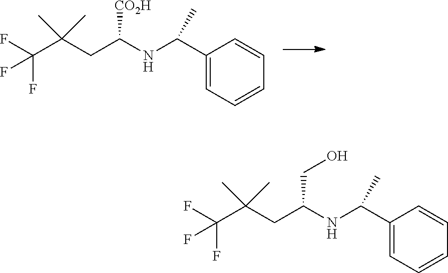 [SMILES: C[C@@H](N[C@H](CC(C)(C)C(F)(F)F)C(=O)O)c1ccccc1 ; C[C@@H](N[C@@H](CO)CC(C)(C)C(F)(F)F)c1ccccc1]

To a suspension of (2R)-5,5,5-trifluoro-4,4-dimethyl-2-[[(1R)-1-phenylethyl]amino]pentanoic acid (hydrochloride salt) (13.04 g, 36.267 mmol) in THF (200 mL) at 35° C. was added LAH in THF (100 mL of 1 M, 100.00 mmol) dropwise. The mixture was stirred at 40° C. for 2 hours, cooled to 10° C. with ice-water bath and diluted with THF (200 mL). A mixture of water (3.8 g) and THF (50 mL) was added dropwise, followed by 25% aqueous NaOH (3.8 g) and water (10 g). The resulting mixture was stirred at rt for 30 min and at 50° C. for 1 h, filtered and washed with warm THF. The filtrate was concentrated to give 12.02 g of product (free amine) as a colorless oil. ¹H NMR (300 MHz, CDCl₃) δ 7.37-7.24 (m, 5H), 3.82 (q, J=6.5 Hz, 1H), 3.72-3.67 (m, 1H), 3.21 (dd, J=10.6, 4.7 Hz, 1H), 2.67 (quin, J=4.6 Hz, 1H), 1.66 (dd, J=14.7, 5.9 Hz, 1H), 1.54-1.45 (m, 1H), 1.36 (d, J=6.5 Hz, 3H), 1.03 (s, 3H), 0.97 (s, 3H). ¹⁹F NMR (282 MHz, CDCl₃) δ −78.83 (s, 3F). The above crude product (12.02 g) was dissolved in diethyl ether (20 mL) and diluted with heptanes (80 mL) and cooled in an ice-water bath. HCl in 1,4-dioxane (10.5 mL of 4 M, 42.000 mmol) was added dropwise. The mixture was stirred at rt for 30 min and filtered to give (2R)-5,5,5-trifluoro-4,4-dimethyl-2-[[(1R)-1-phenylethyl]amino]pentan-1-ol (hydrochloride salt) (11.56 g, 98%) as a white solid. ¹H NMR (300 MHz, DMSO-d₆) δ 9.57 (br. s., 1H), 9.25 (t, J=9.8 Hz, 1H), 7.80-7.59 (m, 2H), 7.53-7.32 (m, 3H), 5.63 (br. s., 1H), 4.58 (t, J=6.3 Hz, 1H), 3.81-3.65 (m, 1H), 3.64-3.51 (m, 1H), 2.91-2.74 (m, 1H), 1.98-1.85 (m, 1H), 1.85-1.74 (m, 1H), 1.63 (d, J=6.8 Hz, 3H), 0.91 (s, 3H), 0.88 (s, 3H). ¹⁹F NMR (282 MHz, DMSO-d₆) δ −77.71 (s, 3F). ESI-MS m/z calc. 289.16534, found 290.2 (M+1)+; Retention time: 2.08 minutes; LC method U.

### Step 6: (2R)-2-Amino-5,5,5-trifluoro-4,4-dimethyl-pentan-1-ol

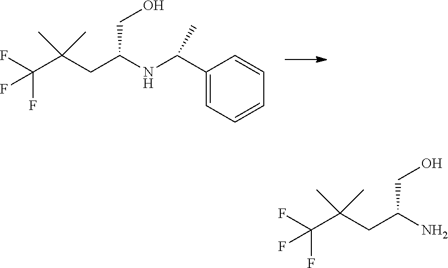 [SMILES: C[C@@H](N[C@@H](CO)CC(C)(C)C(F)(F)F)c1ccccc1 ; CC(C)(C[C@@H](N)CO)C(F)(F)F]

To a solution of (2R)-5,5,5-trifluoro-4,4-dimethyl-2-[[(1R)-1-phenylethyl]amino]pentan-1-ol (hydrochloride salt) (11.56 g, 35.482 mmol) in EtOH (200 mL) was added 10% palladium on carbon, 50% wet (5 g, 2.3492 mmol). The mixture was hydrogenated in a Parr shaker hydrogenation apparatus at 40 psi of hydrogen at rt for 9 hours. More 10% palladium on carbon, 50% wet (1 g, 0.4698 mmol) was added. The mixture was shaken at 40 psi for 7 hours. The mixture was filtered through Celite and washed with EtOH. The filtrate was concentrated. The residue (7.9 g) was triturated with a mixture of 2-methyltetrahydrofuran (28 mL) and heptanes (200 mL) and stirred overnight. The mixture was filtered, and the white solid was dried on vacuum to give (2R)-2-amino-5,5,5-trifluoro-4,4-dimethyl-pentan-1-ol (hydrochloride salt) (7.66 g, 93%) as a white solid. ¹H NMR (300 MHz, DMSO-d₆) δ 8.08 (br. s., 3H), 5.46 (t, J=5.0 Hz, 1H), 3.67-3.52 (m, 1H), 3.43 (dt, J=11.7, 5.8 Hz, 1H), 3.29-3.16 (m, 1H), 1.88-1.73 (m, 1H), 1.72-1.58 (m, 1H), 1.15 (s, 3H), 1.10 (s, 3H). ¹⁹F NMR (282 MHz, DMSO-d₆) δ −78.07 (s, 3F). ESI-MS m/z calc. 185.10275, found 186.2 (M+1)⁺; Retention time: 0.64 minutes; LC method U.

### Example H: Preparation of (2R)-2-amino-3-[1-(trifluoromethyl)cyclopropyl]propan-1-ol

### Step 1: 2-[1-(Trifluoromethyl)cyclopropyl]ethanol

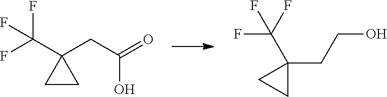 [SMILES: O=C(O)CC1(C(F)(F)F)CC1 ; OCCC1(C(F)(F)F)CC1]

LAH (49.868 g, 1.3139 mol) was added to THF (1700 mL) under nitrogen and the mixture was stirred for 30 minutes before being cooled to 0° C. 2-[1-(trifluoromethyl)cyclopropyl]acetic acid (190.91 g, 1.0107 mol) in THF (500 mL) was added dropwise while controlling the temperature <5° C. The mixture was allowed to warm up to room temperature and stirred for 24 hours. The resulting suspension was cooled to 0° C., water (50 mL) was added very slowly, followed by 15% w/w sodium hydroxide (50 mL) and water (150 mL). The mixture was stirred at 0° C. for 30 minutes, and filtered through Celite pad, the filter cake was washed with THF (2×500 mL). The combined filtrates were evaporated in vacuo to give 2-[1-(trifluoromethyl)cyclopropyl]ethanol (160.27 g, 98%) as amber oil containing ˜5% w/w of THE (by NMR). ¹H NMR (250 MHz, DMSO-d₆) δ 4.57 (t, J=5.2 Hz, 1H), 3.55-3.39 (m, 2H), 1.74 (t, J=7.3 Hz, 2H), 1.00-0.58 (m, 4H).

### Step 2: 2-[1-(Trifluoromethyl)cyclopropyl]acetaldehyde

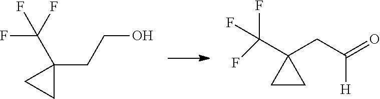 [SMILES: OCCC1(C(F)(F)F)CC1 ; O=CCC1(C(F)(F)F)CC1]

To a solution of 2-[1-(trifluoromethyl)cyclopropyl]ethanol (80 g, 467.1 mmol) in methylene chloride (1.1 L) was stirred at room temperature and treated with Dess-Martin periodinane (250 g, 589.4 mmol) portionwise (exothermic! cooled in ice bath and kept T<15° C.). To the mixture was added water (12 mL, 666.1 mmol) slowly added over 0.5 h (exothermic during addition up to 33° C., kept between 20 and 33° C. by cooling with cold water) giving a thick suspension. After the addition, the pale-yellow fine suspension was stirred at room temperature for 18 hours. The yellow suspension was diluted with diethylether (500 mL) (yellow suspension) and stirred for 30 minutes. The slurry was filtered over Celite and the precipitate washed with 100 mL of Diethylether. diethylether. The organic phase was carefully treated with a saturated aqueous solution of sodium carbonate (500 ml, strong gas evolution, pH ˜10 at the end). The three-phase mixture was stirred at room temperature for 1 hours and the solid was removed by filtration (large glass fritt). The phases (yellow cloudy Diethylether phase, colorless water phase) were separated and the organic phase was washed once more with a saturated aqueous solution of sodium carbonate (250 mL), once with 1M sodium thiosulfate (250 mL) and once with brine (250 mL). The aqueous phases were back extracted once with diethyl ether (150 mL) and the combined organic phases were dried, filtered and evaporated to give 2-[1-(trifluoromethyl)cyclopropyl]acetaldehyde (40 g, 56%) as a yellow liquid.

### Step 3: 2-[[(1R)-1-Phenylethyl]amino]-3-[1-(trifluoromethyl)cyclopropyl]propanenitrile

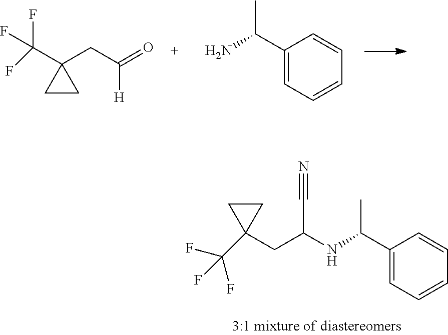

2-[1-(Trifluoromethyl)cyclopropyl]acetaldehyde (102 g, 670.5 mmol) in MeOH (700 mL) was treated with (1R)-1-phenylethanamine (86 mL, 667.1 mmol) and cooled in an ice bath. The solution was treated with acetic acid (38 mL, 668.2 mmol), stirred for 20 min in the ice bath, then solid NaCN (CAUTION, 33 g, 673.4 mmol) was added in one portion and the suspension was stirred in the melting ice bath for 14 hours. The solution was concentrated under reduced pressure the exhaust from the pump was running through a bleach trap) and the residue was extracted with MTBE (1000 mL) and saturated sodium carbonate/water 1:1 (1000 mL) and washed with brine (350 mL). The aqueous phases were back extracted once with MTBE (250 mL) and the combined organic phases were dried, filtered and evaporated to give 2-[[(1R)-1-phenylethyl]amino]-3-[1-(trifluoromethyl)cyclopropyl]propanenitrile (180.8 g, 96%) as 3:1 mixture of diastereomers. ESI-MS m/z calc. 282.13437, found 283.0 (M+1)⁺; Retention time: 1.69 minutes (major isomer) and 1.62 minutes (minor isomer), LC method A.

### Step 4: (2R)-2-[[(1R)-1-Phenylethyl]amino]-3-[1-(trifluoromethyl)cyclopropyl]propenamide

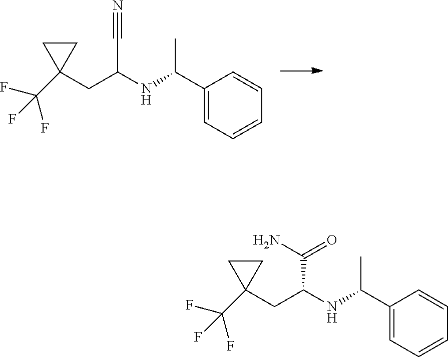 [SMILES: C[C@@H](NC(C#N)CC1(C(F)(F)F)CC1)c1ccccc1 ; C[C@@H](N[C@H](CC1(C(F)(F)F)CC1)C(N)=O)c1ccccc1]

In a 2 L flask equipped with mechanical stirring and a temperature probe, sulfuric acid (285 mL of 18 M, 5.130 mol) was added it was cooled in an ice bath. At an internal temperature of 5° C., a solution of 2-[[(1R)-1-phenylethyl]amino]-3-[1-(trifluoromethyl)cyclopropyl]propanenitrile (180.8 g, 640.4 mmol, 3:1 mixture of diastereomers) in DCM (900 mL) was added dropwise over 20 minutes. The ice bath was removed, and the deep orange emulsion was stirred at room temperature for 18 hours and at 30-40° C. for 2 hours. The deep orange emulsion was carefully added to a mixture of ice and water (2.2 L) under mechanical stirring to give a yellow three phase mixture which was basified by slow addition of ammonium hydroxide (1.33 L of 30% w/w, 10.25 mol) under ice cooling (very exothermic, internal temperature kept between 10 and 25° C. by adding ice). The yellow emulsion was stirred for 10 minutes at room temperature (pH ˜10), diluted with DCM (500 mL) and the phases were separated. The aqueous phase was washed twice more with DCM (400 and 200 mL) and the combined organic phases were washed once with water/brine 1:1 (500 mL). The DCM phase was dried, filtered and evaporated to give crude 2-[[(1R)-1-phenylethyl]amino]-3-[1-(trifluoromethyl)cyclopropyl]propanamide (189.5 g, 99%) as a yellow-orange oil. ESI-MS m/z calc. 300.14496, found 301.0 (M+1)+; Retention time: 1.40 minutes (major isomer) and 1.50 minutes (minor isomer) (3:1 mixture of diastereomers). The product was dissolved in ethanol (1.5 L) and it was treated quickly with HCl (240 mL of 4 M, 960.0 mmol) (4 M in dioxane) and the resulting thick suspension was stirred at room temperature overnight under mechanic stirring. The solid was collected by filtration, washed with cold ethanol and dried under vacuum with a nitrogen bleed at 40-45° C. to give (2R)-2-[[(1R)-1-phenylethyl]amino]-3-[1-(trifluoromethyl)cyclopropyl]propanamide (hydrochloride salt) (147 g, 68%). ¹H NMR (499 MHz, DMSO-d₆) δ 9.74 (d, J=67.9 Hz, 2H), 8.16-7.94 (m, 1H), 7.86 (s, 1H), 7.64-7.51 (m, 2H), 7.51-7.34 (m, 3H), 4.22 (s, 1H), 3.46-3.37 (m, 1H), 2.45 (d, J=15.9 Hz, 1H), 1.85 (dd, J 15.1, 10.4 Hz, 1H), 1.58 (d, J=6.7 Hz, 3H), 0.89 (pd, J=9.6, 9.2, 4.3 Hz, 2H), 0.84-0.66 (m, 2H). ESI-MS m/z calc. 300.14496, found 301.0 (M+1)⁺; Retention time: 1.40 minutes (major isomer) and 1.40 minutes (minor isomer), 97:3 mixture of diastereomers (LC method V).

### Step 5: (2R)-2-[[(1R)-1-Phenylethyl]amino]-3-[1-(trifluoromethyl)cyclopropyl]propanoic acid

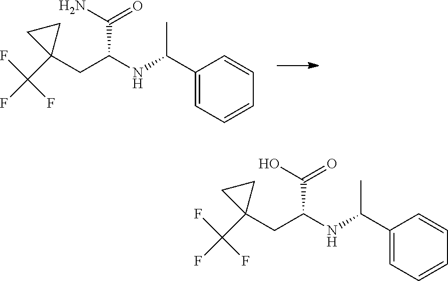 [SMILES: C[C@@H](N[C@H](CC1(C(F)(F)F)CC1)C(N)=O)c1ccccc1 ; C[C@@H](N[C@H](CC1(C(F)(F)F)CC1)C(=O)O)c1ccccc1]

In a 5 L flask equipped with mechanical stirring, (2R)-2-[[(1R)-1-phenylethyl]amino]-3-[1-(trifluoromethyl)cyclopropyl]propanamide (hydrochloride salt) (147 g, 436.5 mmol) was added to acetic acid (735 mL) under stirring and the thick colorless suspension was treated with HCl (1.3 L of 12 M, 15.60 mol). The colorless suspension was carefully heated to 60-65° C. (strong foaming, acetic acid (145 mL) was added) and the suspension was stirred at 60-65° C. for 16 hours. The suspension was then slowly heated to 100° C. (over 4 hours, strong foaming) and the resulting solution was stirred at 100° C. for another 20 hours. The pale-yellow solution was concentrated under reduced pressure at 65° C. to a semisolid mass and it was treated with water (1.5 L). The thick suspension was heated to 70-80° C. and left to cool to room temperature under stirring for 2 h. The solid was collected by filtration, washed with water and sucked dry overnight. The wet solid was further dried under reduced pressure at 50-60° C. for 4 h to give (2R)-2-[[(1R)-1-phenylethyl]amino]-3-[1-(trifluoromethyl)cyclopropyl]propanoic acid (hydrochloride salt) (135 g, 92%) as an off-white solid. ESI-MS m/z calc. 301.12897, found 302.0 (M+1)⁺; Retention time: 1.82 minutes; (LC method V).

### Step 6: (2R)-2-[[(1R)-1-phenylethyl]amino]-3-[1-(trifluoromethyl)cyclopropyl]propan-1-ol

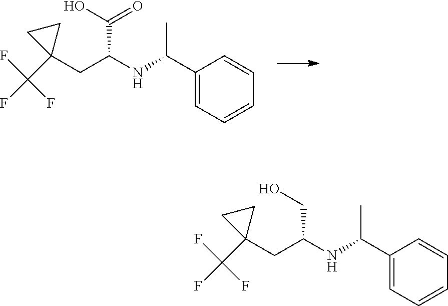 [SMILES: C[C@@H](N[C@H](CC1(C(F)(F)F)CC1)C(=O)O)c1ccccc1 ; C[C@@H](N[C@@H](CO)CC1(C(F)(F)F)CC1)c1ccccc1]

In a 5 L flask equipped with mechanical stirring and under dry nitrogen atmosphere, (2R)-2-[[(1R)-1-phenylethyl]amino]-3-[1-(trifluoromethyl)cyclopropyl]propanoic acid (hydrochloride salt) (135 g, 399.7 mmol) was suspended in THF (2 L) (thick suspension). It was heated to 35-40° C. and LAH (47.3 g, 1.214 mol) (pellets) was slowly added over 1 hour, while keeping the internal temperature between 30 and 40° C. by external cooling. The mixture was stirred for 1 hour at 30-40° C. (almost no hydrogen evolution anymore, grey suspension, most starting material in solution) and it was heated at 50-55° C. for 1 hours. The grey suspension was left stirring in the cooling heating mantel overnight. The grey suspension was cooled in an ice bath and quenched by careful addition of water (44 mL, 2.442 mol), NaOH (41 mL of 6 M, 246.0 mmol) and water (44 mL, 2.442 mol) (high exotherm with first water addition, kept between 5° C. and 30° C. by cooling). The grey suspension was heated to 50-55° C. for 1 hours, by which time a colorless suspension was obtained. The warm suspension was filtered over a pad of Celite covered over magnesium sulfate. The solids were washed with hot THF and evaporated to give crude (2R)-2-[[(1R)-1-phenylethyl]amino]-3-[1-(trifluoromethyl)cyclopropyl]propan-1-ol (121 g, 105%) as an oil. The crude was dissolved in diethyl ether (1 L, clear solution) and slowly treated with HCl (101 mL of 4 M, 404.0 mmol) (4M in dioxane) under cooling. The resulting thick suspension was stirred at room temperature for 1 hour, the solid collected by filtration, washed with diethyl ether and dried under reduced pressure at 40-45° C. with a nitrogen bleed to give (2R)-2-[[(1R)-1-phenylethyl]amino]-3-[1-(trifluoromethyl)cyclopropyl]propan-1-ol (hydrochloride salt) (126.6 g, 98%) as an off-white solid. ¹H NMR (500 MHz, DMSO-d₆) δ 9.34 (s, 2H), 7.66 (d, J=7.4 Hz, 2H), 7.43 (dt, J=25.1, 7.4 Hz, 3H), 5.59 (s, 1H), 4.58 (q, J=6.6 Hz, 1H), 3.83 (d, J=12.6 Hz, 1H), 3.62-3.54 (m, 1H), 2.89 (s, 1H), 2.33-2.24 (m, 1H), 1.67-1.51 (m, 4H), 0.97-0.81 (m, 3H), 0.71 (s, 1H). ESI-MS m/z calc. 287.1497, found 288.0 (M+1)⁺; Retention time: 0.99 minutes (LC method A).

### Step 7: (2R)-2-Amino-3-[1-(trifluoromethyl)cyclopropyl]propan-1-ol

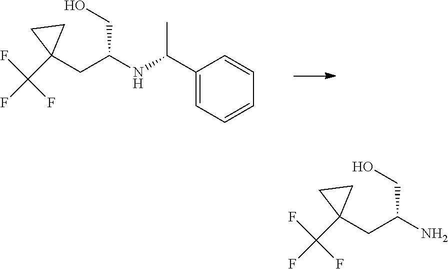 [SMILES: C[C@@H](N[C@@H](CO)CC1(C(F)(F)F)CC1)c1ccccc1 ; N[C@@H](CO)CC1(C(F)(F)F)CC1]

In a 1 L hydrogenation reactor, (2R)-2-[[(1R)-1-phenylethyl]amino]-3-[1-(trifluoromethyl)cyclopropyl]propan-1-ol (hydrochloride salt) (63.3 g, 195.5 mmol) was dissolved in EtOH (630 mL) (under warming), and it was treated with Pd/C (6.3 g of 10% w/w, 5.920 mmol) (12.5 g of 50% water wet) and the reaction was stirred under 2 bar of hydrogen at 40° C. for 24 h. The reaction mixture was filtered over Celite. The pad was washed with ethanol and the colorless filtrate was evaporated to a solid mass, which was triturated with diethyl ether. The suspension was stirred at room temperature for 1 h. The solid was filtered, washed with plenty of diethyl ether and dried to give (2R)-2-amino-3-[1-(trifluoromethyl)cyclopropyl]propan-1-ol (hydrochloride salt) (41.8 g, 97%) as an off-white solid. ¹H NMR (500 MHz, DMSO-d₆) δ 8.18 (s, 3H), 5.45 (t, J=4.9 Hz, 1H), 3.71 (dt, J=11.6, 3.9 Hz, 1H), 3.55 (dt, J=11.2, 5.4 Hz, 1H), 3.24 (h, J=4.7 Hz, 1H), 2.08 (dd, J=15.1, 5.4 Hz, 1H), 1.69 (dd, J=15.1, 9.4 Hz, 1H), 0.97 (h, J=6.5, 5.9 Hz, 2H), 0.86 (s, 2H). ESI-MS m/z calc. 183.0871, found 184.0 (M+1)⁺; Retention time: 0.65 minutes; LC method A.

### Example I: Preparation of (2R)-4-methyl-2-(spiro[2.3]hexan-5-ylamino)pentan-1-ol

### Step 1: (2R)-4-Methyl-2-(spiro[2.3]hexan-5-ylamino)pentan-1-ol

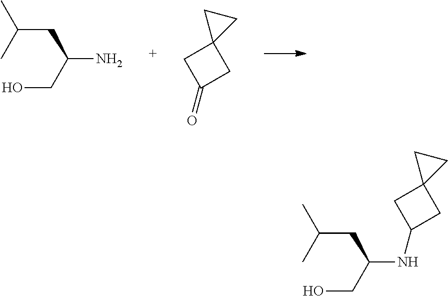

A mixture of spiro [2.3]hexan-5-one (100 g, 1.040 mol) and (2R)-2-amino-4-methyl-pentan-1-ol (123.5 g, 1.054 mol) in DCE (1.5 L) was stirred at ambient temperature for 1 hour. To the mixture was added sodium triacetoxyborohydride (228 g, 1.076 mol) portionwise. The mixture was stirred at ambient temperature for 18 hours. The reaction mixture was diluted with HCl (1.1 L of 2 M, 2.200 mol) until pH was ˜1. The aqueous phase was separated and the organic phase extracted with HCl (600 mL of 2 M, 1.200 mol). The organic phase (DCE) was separated and the aqueous layer was basified with NaOH (550 g of 50% w/w, 6.875 mol) affording a solution at ˜ pH 12. The mixture was extracted 2× with EtOAc (1 L) and the combined organic phases were washed with brine (150 mL), dried over MgSO₄, filtered and concentrated in vacuo affording a clear oil. Used without further purification. (2R)-4-methyl-2-(spiro[2.3]hexan-5-ylamino)pentan-1-ol (160.7 g, 78%). ESI-MS m/z calc. 197.17796, found 198.2 (M+1)⁺; Retention time: 0.54 minutes (LC method A)

### Step 2: (2R)-4-Methyl-2-(spiro[2.3]hexan-5-ylamino)pentan-1-ol (hydrochloride salt)

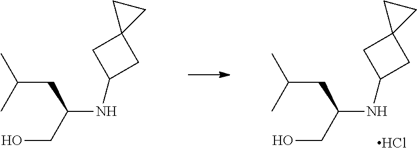

HCl (354 mL of 4 M, 1.416 mol) (4 M in dioxane) was added to a stirring (mechanical) solution of (2R)-4-methyl-2-(spiro[2.3]hexan-5-ylamino)pentan-1-ol (254 g, 1.287 mol) in diethyl ether (2.286 L) in an ice/ice water bath over 20 minutes, keeping the internal temp between 10° C. and 22° C. After the addition was complete, the solution was stirred at rt for 1.5 hours. The product was filtered out and rinsed with 2000 mL diethyl ether. The exact same process was repeated again on the exact same scale (a total of 508 g of amino alcohol SM was used). The product was dried under vacuum at 35° C. overnight and gave 562.3 g. (2R)-4-methyl-2-(spiro[2.3]hexan-5-ylamino)pentan-1-ol (Hydrochloride salt) (562.3 g, 93%). ¹H NMR (500 MHz, DMSO-d₆) δ 9.17-8.84 (m, 2H), 5.38 (s, 1H), 3.99 (p, J=7.2 Hz, 1H), 3.70-3.60 (m, 1H), 3.55-3.45 (m, 1H), 3.03-2.91 (m, 1H), 2.63-2.54 (m, 2H), 2.20-2.05 (m, 2H), 1.73-1.60 (m, 1H), 1.60-1.48 (m, 1H), 1.43-1.30 (m, 1H), 0.93-0.83 (m, 6H), 0.55-0.45 (m, 2H), 0.45-0.36 (m, 2H).

### Example J: Preparation of 4,6-dichloro-5-isopropoxy-pyrimidin-2-amine

### Step 1: Diethyl 2-isopropoxypropanedioate

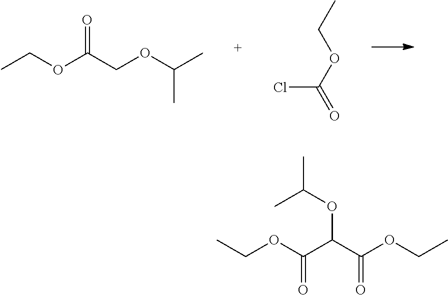

To a solution of diisopropylamine (11.119 g, 15.4 mL, 109.88 mmol) in THF (100 mL) at −78° C. was added n-butyllithium (41.3 mL of 2.5 M, 103.25 mmol). The mixture was stirred at 0° C. for 15 min and cooled to −78° C., A solution of ethyl 2-isopropoxyacetate (10.83 g, 68.899 mmol) in THF (25 mL) was added dropwise at a temperature below −74° C. The mixture was stirred for 10 min. A solution of ethyl chloroformate (7.9450 g, 7 mL, 73.210 mmol) in THF (15 mL) was added dropwise at −73° C. The mixture was stirred for 10 min and allowed to warm up to −50° C. and stirred for 5 min. Solid ammonium chloride (7.37 g, 2 eq) was added. The mixture was stirred at −70° C. for 5 minutes and crushed ice (20 g) was added. The mixture was allowed to warm up to rt and the organic layer was separated and concentrated after drying over sodium sulfate. The yellow oil residue was purified by flash chromatography on silica gel (220 g) using a 0-25% gradient of ethyl acetate in heptanes over 22 column volumes to give the title compound diethyl 2-isopropoxypropanedioate (9.26 g, 55% yield) as a yellow oil. ¹H NMR (300 MHz, CDCl₃) δ ppm 1.24 (d, J=5.9 Hz, 6H), 1.29 (t, J=7.2 Hz, 6H), 3.69-3.83 (m, 1H), 4.19-4.33 (m, 4H), 4.54 (s, 1H). ESI-MS m/z calc. 218.12, found 241.1 (M+Na)⁺. Retention time 1.78 minutes.

### Step 2: 2-Amino-5-isopropoxy-pyrimidine-4,6-diol

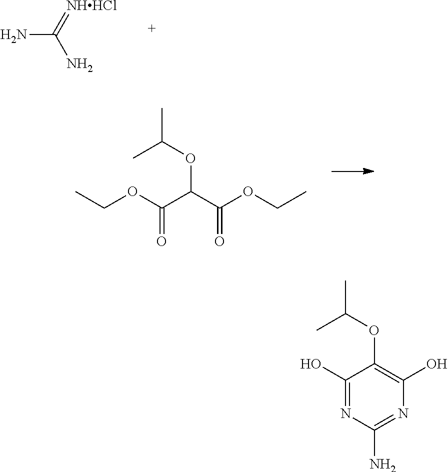

To a 1000-mL three neck flask was added absolute ethanol (215 mL) and solid chunks of sodium metal (4.13 g, 179.64 mmol) were added gradually and the mixture was stirred until all solids dissolved. Once cooled back to around 25° C., guanidine (hydrochloride salt) (6.3 g, 65.947 mmol) and diethyl 2-isopropoxypropanedioate (13.33 g, 59.856 mmol) were successively added. An additional amount of ethanol (70 mL) was added and the reaction was stirred at reflux overnight. Once cooled to room temperature, the crude mixture was concentrated under reduced pressure to remove most of the ethanol. Water (220 mL) was added until dissolution, the resulting solution was cooled in an ice bath and acidified to pH of 5 using acetic acid (120-140 mL). The solids were filtered and washed with water. The solid was dried under reduced pressure and under high vacuum to give 2-amino-5-isopropoxy-pyrimidine-4,6-diol hydrate (7.125 g, 54% yield) as a light pink solid. ¹H NMR (300 MHz, DMSO-d₆) δ ppm 1.08 (d, J=6.2 Hz, 6H), 4.01-4.14 (m, 1H), 6.81 (br. s, 2H), 10.82 (br. s, 2H). ESI-MS m/z calc. 185.08, found 186.1 (M+1)⁺; Retention time: 0.41 minutes (LC method C).

### Step 3: 4,6-Dichloro-5-isopropoxy-pyrimidin-2-amine

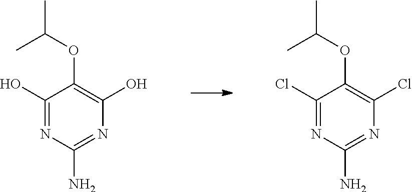

A vigorously stirred suspension of 2-amino-5-isopropoxy-pyrimidine-4,6-diol (7.12 g, 35.508 mmol) in phosphorus oxychloride (53.627 g, 32.6 mL, 349.75 mmol) was heated to 90° C. and N,N-diethylaniline (7.4400 g, 8 mL, 49.855 mmol) was added dropwise. The suspension was stirred for one to three hours at 105° C., then upon completion was allowed to cool to rt. Excess phosphorus oxychloride was quenched by careful addition of 450 mL of aqueous 1.0 N NaOH, while keeping internal temperature below 60° C. At the end of the addition the pH reached about 1-2, then it was adjusted to pH=5 by careful addition of a 25% aqueous solution of NaOH. The resultant mixture was added to an extraction funnel and extracted with dichloromethane (3×120 mL). The combined organic phases were washed with brine (1×80 mL), then dried by filtration over a cotton pad with anhydrous sodium sulfate, then concentrated by rotary evaporation. The residue was purified by flash chromatography using a gradient of ethyl acetate (0-30%) in heptanes on a 120 g column (silica gel) to give 4,6-dichloro-5-isopropoxy-pyrimidin-2-amine (3.97 g, 50%). ¹H NMR (300 MHz, CDCl₃) δ ppm 1.36 (d, J=6.2 Hz, 6H), 4.42 (dq, J=12.3, 6.2 Hz, 1H), 5.07 (br. s, 2H). ESI-MS m/z calc. 221.0123, found 222.1 (M+1)⁺; Retention time: 1.77 minutes (LC method P).

### Example K: Preparation of 3-[[4-Chloro-6-(2,6-dimethylphenyl)-5-fluoro-pyrimidin-2-yl]sulfamoyl]benzoic acid

### Step 1: 2-Amino-5-fluoro-pyrimidine-4,6-diol

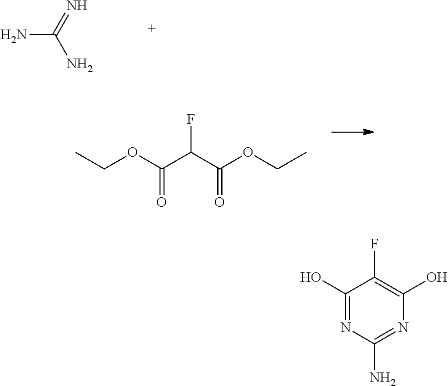

To a 2 L flask charged with ethanol (750 mL), solid chunks of sodium metal (21.5 g, 935.20 mmol) were added gradually and carefully and the mixture was stirred until completely dissolved. Once cooled back to room temperature guanidine (hydrochloride salt) (34.5 g, 361.14 mmol) and diethyl 2-fluoropropanedioate (50 g, 280.65 mmol) were successively added and the reaction was heated at 80° C. for 17 hours. Once cooled to room temperature, the crude mixture was concentrated under reduced pressure to remove most of the ethanol. Water was added to complete dissolution, the resulting solution was cooled in an ice bath and acidified to pH of 1-2 using concentrated HCl. The solids were filtered and washed with water (2×200 mL) then with acetone (2×50 mL) and dried under high vacuum to provide 2-amino-5-fluoro-pyrimidine-4,6-diol hydrate (41.8 g, 91%)) as a pink solid. ¹H NMR (300 MHz, DMSO-d₆) ppm 6.53 (br. s., 1H), 10.97 (br. s., 1H). 19F NMR (282 MHz, DMSO-d₆) ppm −196.1 (br. s., 1F). ESI-MS m/z calc. 145.0288, found 146.1 (M+1)⁺; Retention time: 0.2 minutes (LC method P).

### Step 2: 4,6-Dichloro-5-fluoro-pyrimidin-2-amine

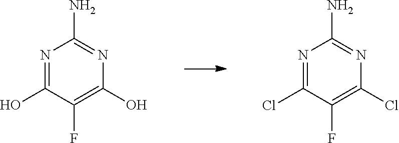 [SMILES: Nc1nc(O)c(F)c(O)n1 ; Nc1nc(Cl)c(F)c(Cl)n1]

A solution of 2-amino-5-fluoro-pyrimidine-4,6-diol hydrate (21.09 g, 126.46 mmol) in phosphorus oxychloride (101.99 g, 62 mL, 665.16 mmol) was heated to 90° C. and N,N-diethylaniline (25.326 g, 27 mL, 169.71 mmol) was added slowly. The reaction was then heated for 3 hours at 105° C. The solution was poured in water and neutralized to pH ˜5 with 50% aqueous sodium hydroxide solution and refluxed for 1h. The solution was cooled on ice and the precipitate was filtered and dried. The solid was triturated in dichloromethane (˜150 mL), filtered and dried. The solid obtained was then triturated in a mix of acetone/heptanes (1:1, 40 mL), filtered and dried provide the desired 4,6-dichloro-5-fluoro-pyrimidin-2-amine (13.75 g, 59%) as a yellow solid. ¹H NMR (300 MHz, DMSO-d₆) δ 7.44 (br. s., 2H). ¹⁹F NMR (282 MHz, DMSO-d₆) δ −149.01 (s, 1F). ESI-MS m/z calc. 180.961, found 182.0 (M+1)⁺; Retention time: 2.17 minutes. LC method U.

### Step 3: tert-Butyl N-(4,6-dichloro-5-fluoro-pyrimidin-2-yl)carbamate

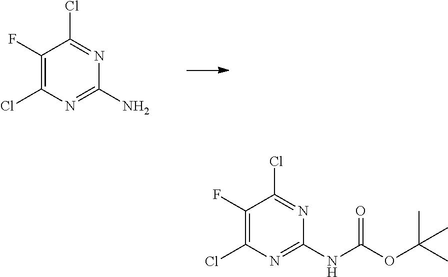 [SMILES: Nc1nc(Cl)c(F)c(Cl)n1 ; CC(C)(C)OC(=O)Nc1nc(Cl)c(F)c(Cl)n1]

To a stirring solution of 4,6-dichloro-5-fluoro-pyrimidin-2-amine (5.153 g, 27.495 mmol) and Boc anhydride (8.43 g, 38.626 mmol) in anhydrous THF (100 mL) at −78° C. under nitrogen was dropwise added a solution of LiHMDS (50 mL of 1.3 M, 65.000 mmol) in THF. After the addition was complete, the reaction mixture was stirred at this temperature for 2 hours. The reaction was quenched cold with saturated aqueous ammonium chloride (20 mL) and allowed to warm up to room temperature. The product was extracted with ethyl acetate (3×30 mL). The combined organic layers were washed with brine (30 mL), dried over anhydrous sodium sulfate and concentrated. The crude was purified by silica gel chromatography using 0-15% ethyl acetate in hexane to afford tert-butyl N-(4,6-dichloro-5-fluoro-pyrimidin-2-yl)carbamate (6.632 g, 86%) as a white solid. ¹H NMR (500 MHz, DMSO-d₆) δ 10.74 (s, 1H), 1.45 (s, 9H).

### Step 4: tert-Butyl N-[4-chloro-6-(2,6-dimethylphenyl)-5-fluoro-pyrimidin-2-yl]carbamate

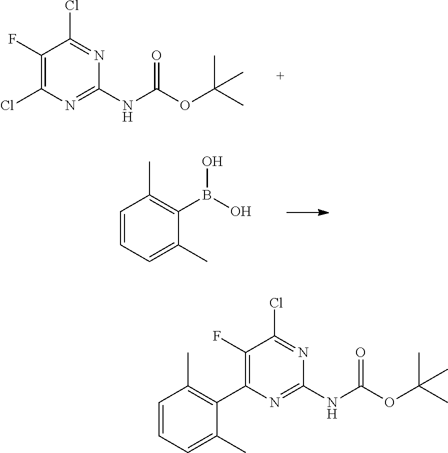 [SMILES: CC(C)(C)OC(=O)Nc1nc(Cl)c(F)c(Cl)n1 ; Cc1cccc(C)c1B(O)O ; Cc1cccc(C)c1-c1nc(NC(=O)OC(C)(C)C)nc(Cl)c1F]

A stirring solution of tert-butyl N-(4,6-dichloro-5-fluoro-pyrimidin-2-yl)carbamate (6.815 g, 22.467 mmol) and (2,6-dimethylphenyl)boronic acid (3.06 g, 20.402 mmol) in a mixture of 1,2-dimethoxyethane (55 mL) and water (15 mL) at room temperature was degassed with nitrogen for 30 minutes. Under nitrogen, cesium carbonate (18.3 g, 56.166 mmol) and [1,1′-bis(diphenylphosphino)ferrocene]dichloropalladium(II) (1.65 g, 2.2550 mmol) were added. The reaction mixture was heated to 80° C. for 4 hours. After cooling to room temperature, water (150 mL) was added and the product was extracted with ethyl acetate (3×150 mL). The combined organic layers were washed with brine (50 mL), dried over anhydrous sodium sulfate and concentrated. The crude was purified by silica gel chromatography using 0-5% hexanes-ethyl acetate to afford tert-butyl N-[4-chloro-6-(2,6-dimethylphenyl)-5-fluoro-pyrimidin-2-yl]carbamate (2.43 g, 29%) as white solid. ESI-MS m/z calc. 351.115, found 352.4 (M+1)⁺; Retention time: 6.51 minutes, LC method S.

### Step 5: 4-Chloro-6-(2,6-dimethylphenyl)-5-fluoro-pyrimidin-2-amine

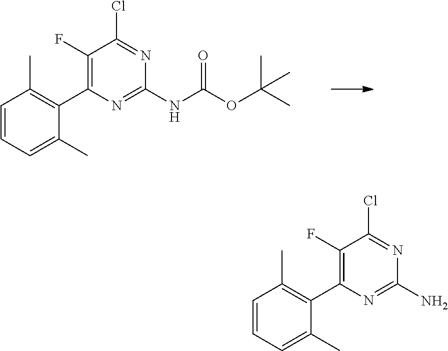 [SMILES: Cc1cccc(C)c1-c1nc(NC(=O)OC(C)(C)C)nc(Cl)c1F ; Cc1cccc(C)c1-c1nc(N)nc(Cl)c1F]

To a stirring solution of tert-butyl N-[4-chloro-6-(2,6-dimethylphenyl)-5-fluoro-pyrimidin-2-yl]carbamate (2.43 g, 6.4238 mmol) in DCM (23 mL) at room temperature was added a solution of HCl (6.5 mL of 4 M, 26.000 mmol) in 1,4-dioxane. The reaction mixture was stirred at this temperature for 2 hours. The reaction mixture was evaporated to dryness. The obtained white solid was resuspended in saturated aqueous sodium bicarbonate (100 mL) and stirred at room temperature for 15 minutes. The product was extracted with ethyl acetate (3×100 mL). The combined organic layers were washed with brine (50 mL), dried over anhydrous sodium sulfate and concentrated. The crude was purified by silica gel chromatography using 0-15% hexanes-ethyl acetate to afford 4-chloro-6-(2,6-dimethylphenyl)-5-fluoro-pyrimidin-2-amine (1.42 g, 83%) as white solid. ESI-MS m/z calc. 251.0626, found 252.3 (M+1)⁺; Retention time: 5.06 minutes, LC method S.

### Step 6: Methyl 3-[[4-chloro-6-(2,6-dimethylphenyl)-5-fluoro-pyrimidin-2-yl]sulfamoyl]benzoate

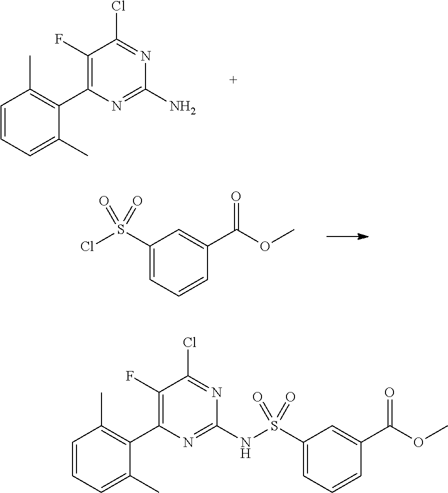 [SMILES: Cc1cccc(C)c1-c1nc(N)nc(Cl)c1F ; COC(=O)c1cccc(S(=O)(=O)Cl)c1 ; COC(=O)c1cccc(S(=O)(=O)Nc2nc(Cl)c(F)c(-c3c(C)cccc3C)n2)c1]

To a stirring solution of 4-chloro-6-(2,6-dimethylphenyl)-5-fluoro-pyrimidin-2-amine (2.54 g, 10.092 mmol) and methyl 3-chlorosulfonylbenzoate (4.25 g, 18.112 mmol) in anhydrous THF (70 mL) at 0° C. under nitrogen was dropwise added a solution of lithium tert-amoxide (5.8400 g, 20 mL of 40% w/w, 24.830 mmol) in heptanes. After the addition was complete, the reaction mixture was stirred at this temperature for 1 hour. The reaction was quenched cold with 1 M aqueous HCl (180 mL) and then allowed to warm up to room temperature. Volatiles were removed under vacuum and the product was extracted with ethyl acetate (3×100 mL). The combined organic layers were washed with brine (60 mL), dried over anhydrous sodium sulfate and concentrated. The crude was purified by silica gel chromatography using 0-20% hexanes-ethyl acetate. The obtained product was triturated with hexanes (100 mL), collected by filtration and dried under vacuum to afford methyl 3-[[4-chloro-6-(2,6-dimethylphenyl)-5-fluoro-pyrimidin-2-yl]sulfamoyl]benzoate (4.381 g, 93%) as white solid. ESI-MS m/z calc. 449.0612, found 450.1 (M+1)⁺; Retention time: 2.72 minutes, LC method T. ¹H NMR (500 MHz, DMSO-d₆) δ 12.49 (s, 1H), 8.43 (t, J=1.8 Hz, 1H), 8.21 (dt, J=7.8, 1.4 Hz, 1H), 8.14 (dt, J=8.0, 1.5 Hz, 1H), 7.73 (t, J=7.8 Hz, 1H), 7.32 (t, J=7.6 Hz, 1H), 7.14 (d, J=7.6 Hz, 2H), 3.82 (s, 3H), 1.85 (s, 6H).

### Step 7: 3-[[4-Chloro-6-(2,6-dimethylphenyl)-5-fluoro-pyrimidin-2-yl]sulfamoyl]benzoic acid

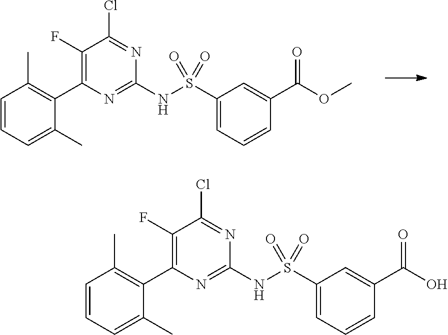 [SMILES: COC(=O)c1cccc(S(=O)(=O)Nc2nc(Cl)c(F)c(-c3c(C)cccc3C)n2)c1 ; Cc1cccc(C)c1-c1nc(NS(=O)(=O)c2cccc(C(=O)O)c2)nc(Cl)c1F]

To a stirring solution of methyl 3-[[4-chloro-6-(2,6-dimethylphenyl)-5-fluoro-pyrimidin-2-yl]sulfamoyl]benzoate (490 mg, 1.0892 mmol) in THF (9 mL) at room temperature was added an aqueous solution of NaOH (4.5 mL of 1 M, 4.5000 mmol). The reaction mixture was stirred at this temperature for 2 hours. Water was added (20 mL), and the reaction mixture was acidified with 2 M aqueous HCl to pH ˜1. The product was extracted with ethyl acetate (3×40 mL). The combined organic layers were washed with brine (30 mL), dried over anhydrous sodium sulfate and concentrated to afford 3-[[4-chloro-6-(2,6-dimethylphenyl)-5-fluoro-pyrimidin-2-yl]sulfamoyl]benzoic acid (463 mg, 93%) as white solid. ESI-MS m/z calc. 435.0456, found 436.4 (M+1)⁺; Retention time: 5.21 minutes; LC method S.

### Example L: Preparation of methyl 6-chlorosulfonylpyridine-2-carboxylate

### Step 1: Methyl 6-benzylsulfanylpyridine-2-carboxylate

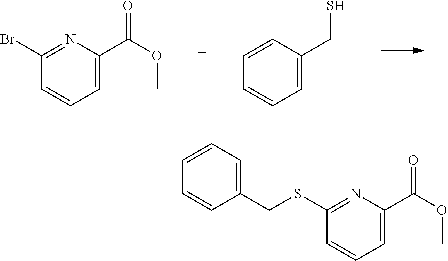 [SMILES: COC(=O)c1cccc(Br)n1 ; SCc1ccccc1 ; COC(=O)c1cccc(SCc2ccccc2)n1]

To a solution of phenylmethanethiol (28.408 g, 26.800 mL, 228.72 mmol) in THE (600 mL) was added NaH (11.200 g, 60% w/w, 280.03 mmol) in a few portions at 0° C. The slurry was warmed to room temperature and stirred for 30 min, then methyl 6-bromopyridine-2-carboxylate (50 g, 231.45 mmol) was added as a single portion. After 3 hours, the reaction was diluted with ether (800 mL) and quenched with water (400 mL) and saturated sodium bicarbonate (50 mL). The layers were separated, and the organic layer was washed with brine, dried over sodium sulfate, and concentrated under reduced pressure to yield methyl 6-benzylsulfanylpyridine-2-carboxylate (56.35 g, 89%) as a yellow oil. ¹H NMR (500 MHz, DMSO-d₆) δ 7.84-7.77 (m, 1H), 7.77-7.73 (m, 1H), 7.52 (m, 1H), 7.48 (d, J=7.8 Hz, 2H), 7.28 (t, J=7.2, 7.2 Hz, 2H), 7.24-7.18 (m, 1H), 4.44 (s, 2H), 3.90 (d, J=1.2 Hz, 3H). ESI-MS m/z calc. 259.0667, found 260.1 (M+1)⁺; Retention time: 3.2 minutes; LC method T.

### Step 2: Methyl 6-chlorosulfonylpyridine-2-carboxylate

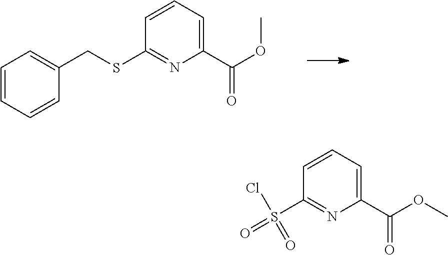 [SMILES: COC(=O)c1cccc(SCc2ccccc2)n1 ; COC(=O)c1cccc(S(=O)(=O)Cl)n1]

A solution of methyl 6-benzylsulfanylpyridine-2-carboxylate (121.62 g, 431.47 mmol) in DCM (950 mL) and DI water (300 mL) was cooled in a −1-0° C. ice bath and, with vigorous stirring, sulfuryl chloride (228.14 g, 140 mL, 1.6396 mol) was added dropwise while the temperature was maintained below 5° C. After the addition, the organic phase was separated, washed with DI water (2×500 mL), dried over anhydrous sodium sulfate, filtered and concentrated under vacuum. The residue was dissolved in DCM (500 mL). Hexanes (1000 mL) was added and the DCM was slowly evaporated off. The white precipitate was filtered by vacuum and the solids were washed with Hexanes (2×500 mL). The filtered solids were collected. The residue solids in the filtrate were filtered and dissolved in DCM (500 mL). The DCM solution was transferred to a 1 L round-bottom flask and concentrated under vacuum. The residue was dissolved in DCM (200 mL). Hexanes (600 mL) was added and the DCM was slowly evaporated off. The white precipitation was filtered by vacuum and the solids were washed with hexanes (2×500 mL) After drying, methyl 6-chlorosulfonylpyridine-2-carboxylate (56.898 g, 55%) was isolated. ¹H NMR (500 MHz, Chloroform-d) δ 8.48 (dd, J=7.8, 1.1 Hz, 1H), 8.31 (dd, J=7.9, 1.1 Hz, 1H), 8.25 (t, J=7.8 Hz, 1H), 4.08 (s, 3H). ESI-MS m/z calc. 234.97061, found 236.1 (M+1)⁺; Retention time: 1.74 minutes; LC method T.

### Example M: Preparation of 4-isopropylpyrimidine-2-carbaldehyde

### Step 1: 2-Iodo-4-isopropyl-pyrimidine

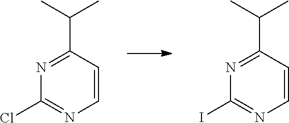

Hydriodic acid (57 wt. % in water) (2 mL of 57% w/v, 8.9124 mmol), precooled to 0° C., was added to solid 2-chloro-4-isopropyl-pyrimidine (500 mg, 3.1926 mmol) in a 10 mL round-bottomed flask. The mixture was kept and vigorously stirred at 0° C. for 2h. The light brownish green suspension was quickly neutralized at 0° C. with a saturated aqueous solution of potassium carbonate (60 mL), 1N NaOH (8 mL, to reach pH=9) and decolorized with potassium disulfite at 0° C. The aqueous solution was extracted with DCM (5×30 mL), dried over magnesium sulfate, filtered, and evaporated under reduced pressure to give 2-iodo-4-isopropyl-pyrimidine (798 mg, 96%) as a light yellow oil. ¹H NMR (400 MHz, CDCl₃) δ 8.31 (d, J=5.1 Hz, 1H), 7.16 (d, J=5.1 Hz, 1H), 3.05-2.89 (m, 1H), 1.29 (s, 3H), 1.27 (s, 3H). ESI-MS m/z calc. 247.98105, found 249.2 (M+1)⁺; Retention time: 1.71 minutes; LC method X.

### Step 2: 4-Isopropylpyrimidine-2-carbaldehyde

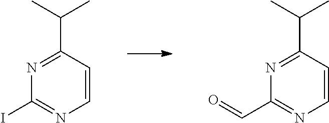

A 10 mL flask equipped with a stirring bar was charged with a solution of 2-iodo-4-isopropyl-pyrimidine (200 mg, 0.8062 mmol) in THF (2.7 mL) under argon atmosphere. isopropylmagnesium chloride lithium chloride complex solution (in THF) (0.7 mL of 1.3 M, 0.9100 mmol) was added dropwise at 0-5° C. The reaction mixture was stirred at 0° C. for 1 h and then ethyl formate (66.024 mg, 72 μL, 0.8913 mmol) was added. The reaction was allowed to warm to rt and stirred overnight. The mixture was quenched with aqueous saturated NH₄Cl (10 mL) and extracted with DCM (3×20 mL). The combined organic extracts were washed brine (40 mL), dried with sodium sulfate and concentrated under a reduced pressure. The residue was purified by flash chromatography using a 24 g cartridge, eluting with diethyl ether (100%) to give 4-isopropylpyrimidine-2-carbaldehyde (85 mg, 35%) as a colorless oil. ¹H NMR (400 MHz, CDCl₃) δ 10.11 (s, 1H), 8.88-8.84 (m, 1H), 7.38-7.35 (m, 1H), 3.23-3.12 (m, 1H), 1.39-1.37 (m, 3H), 1.37-1.36 (m, 3H). ESI-MS m/z calc. 150.07932, found 151.2 (M+1)⁺; Retention time: 1.15 minutes; LC method X.

### Example N: Preparation of 6-[2,2-Dimethylpropyl(methyl)amino]pyridine-2-carbaldehyde

### Step 1: 6-Bromo-N-(2,2-dimethylpropyl)-N-methyl-pyridin-2-amine

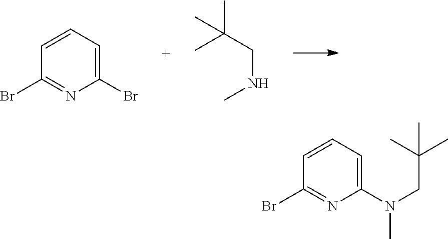

To a solution of N-methyl neopentylamine (hydrochloride salt) (1.3 g, 9.4442 mmol) in DMF (30 mL) was added 2,6-dibromopyridine (2 g, 8.4427 mmol) and potassium carbonate (234 mg, 1.6931 mmol) and the mixture was stirred for 48 h at 120-130° C. The reaction mixture was partitioned between water (100 mL) and ethyl acetate (150 mL). The aqueous layer was back-extracted with ethyl acetate (2×100 mL). The organic phase was washed with a 1:1 v/v mixture of brine and water (4×80 mL), dried over sodium sulfate and filtered and concentrated to dryness. The crude was purified by reverse phase chromatography (C₁₈, 80 g column) eluting with 10% to 100% methanol in water to give 6-bromo-N-(2,2-dimethylpropyl)-N-methyl-pyridin-2-amine (1.68 g, 77%) as a pale-yellow oil. ¹H NMR (400 MHz, CDCl₃) δ 7.22 (dd, J=8.3, 7.6 Hz, 1H), 6.65 (d, J=7.3 Hz, 1H), 6.41 (d, J=8.6 Hz, 1H), 3.37 (s, 2H), 3.07 (s, 3H), 0.97 (s, 9H). ESI-MS m/z calc. 256.05753, found 257.2 (M+1)⁺; Retention time: 2.15 minutes; LC method X.

### Step 2: 6-[2,2-Dimethylpropyl(methyl)amino]pyridine-2-carbaldehyde

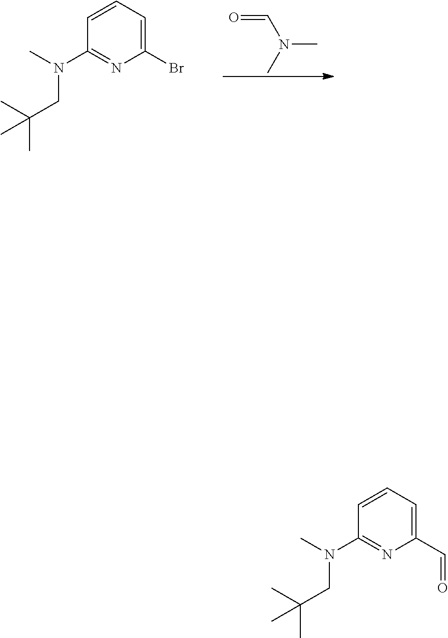

To a solution of 6-bromo-N-(2,2-dimethylpropyl)-N-methyl-pyridin-2-amine (1.68 g, 6.5326 mmol) in THF (13.6 mL) was dropwise added n-butyllithium solution in hexane (3.2 mL of 2.5 M, 8.0000 mmol) at −78° C. After stirring for 1 hour at this temperature, N,N-dimethylformamide (877.92 mg, 0.93 mL, 12.011 mmol) was slowly added and the reaction mixture was stirred for 1 hour at −78° C. The reaction mixture was warmed to room temperature and quenched with a saturated aqueous solution of ammonium chloride (10 mL) and extracted with ethyl acetate (3×20 mL). The organic phase was washed with brine (2×15 mL), dried over sodium sulfate, filtered and concentrated under reduced pressure. The crude was purified by flash chromatography on silica gel (SNAP 100 g) using a gradient of 0% to 10% of ethyl acetate in heptanes to give 6-[2,2-dimethylpropyl(methyl)amino]pyridine-2-carbaldehyde (860 mg, 62%) as a yellow oil. ¹H NMR (400 MHz, CDCl₃) δ 9.89 (d, J=0.7 Hz, 1H), 7.56 (t, J=7.6 Hz, 1H), 7.20 (d, J=7.1 Hz, 1H), 6.75 (d, J=8.6 Hz, 1H), 3.50 (s, 2H), 3.16 (s, 3H), 1.00 (s, 9H). ESI-MS m/z calc. 206.1419, found 207.2 (M+1)⁺; Retention time: 4.6 minutes; LC method Y.

### Example O: Preparation of 5-(3,3-dimethylpyrrolidin-1-yl)pyrimidine-2-carbaldehyde

### Step 1: 5-(3,3-Dimethylpyrrolidin-1-yl)pyrimidine-2-carbonitrile

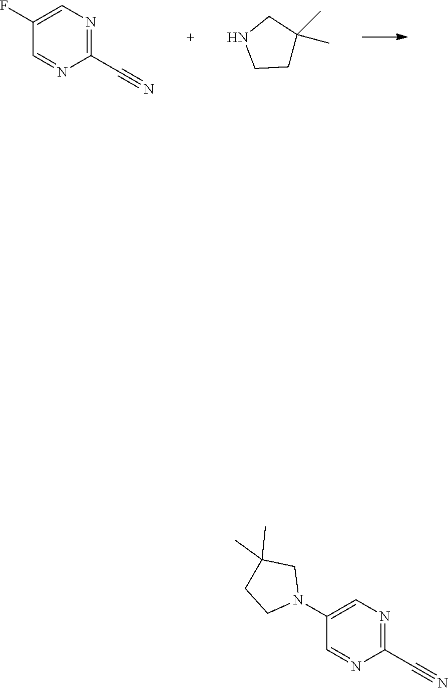

To a stirred solution of 5-fluoropyrimidine-2-carbonitrile (10 g, 77.181 mmol) and 3,3-dimethylpyrrolidine (hydrochloride salt) (13 g, 95.846 mmol) in DMF (100 mL) was added Cesium carbonate (63 g, 193.36 mmol) at rt and stirred for 6 hours (The reaction was exothermic in the beginning). Water (400 mL) was added and the resulting precipitate was filtered. The cake was washed with water and dissolved in DCM (500 mL). The DCM layer was washed with brine, dried over sodium sulfate, filtered and concentrated in vacuo. The resulting beige solid was triturated with hexanes and filtered. The solid was washed with hexanes and dried in vacuo to give 5-(3,3-dimethylpyrrolidin-1-yl)pyrimidine-2-carbonitrile (15.1 g, 92%) as a white solid. ESI-MS m/z calc. 202.12184, found 203.5 (M+1)⁺; Retention time: 4.67 minutes; LC method S.

### Step 2: 5-(3,3-Dimethylpyrrolidin-1-yl)pyrimidine-2-carbaldehyde

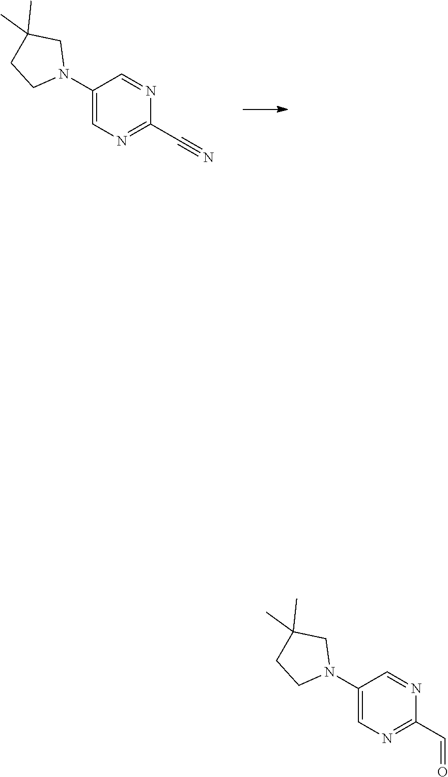

To a stirred solution of 5-(3,3-dimethylpyrrolidin-1-yl)pyrimidine-2-carbonitrile (14.7 g, 69.046 mmol) in THF (300 mL) was added DIBAL in toluene (85 mL of 1 M, 85.000 mmol) at −78° C. dropwise over 30 minutes and stirred for 2 hours. The reaction mixture was quenched with 500 mL of saturated aqueous sodium potassium tartrate solution (Rochelle's salt) and stirred for 30 minutes while being allowed to warm to rt. The layers were separated, and the aqueous layer was extracted with EtOAc (3×300 mL). The combined organic layer was washed with brine, dried over sodium sulfate, filtered and concentrated in vacuo. The resulting red solid was purified by flash chromatography (silica gel, 220 g, loaded in DCM, eluted with 3% MeOH in DCM). The desired product fractions were combined and concentrated in vacuo to give 5-(3,3-dimethylpyrrolidin-1-yl)pyrimidine-2-carbaldehyde (8.08 g, 56%) as an orange solid. ¹H NMR (500 MHz, DMSO-d₆) δ 9.73 (s, 1H), 8.23 (s, 2H), 3.52 (t, J=7.0, 7.0 Hz, 2H), 3.21 (s, 2H), 1.80 (t, J=7.0, 7.0 Hz, 2H), 1.10 (s, 6H). ESI-MS m/z calc. 205.1215, found 206.2 (M+1)⁺; Retention time: 1.69 minutes; LC method W.

### Example P: Preparation of 5-[2-Methoxyethyl(methyl)amino]pyrimidine-2-carbaldehyde

### Step 1: 5-[2-Methoxyethyl(methyl)amino]pyrimidine-2-carbaldehyde

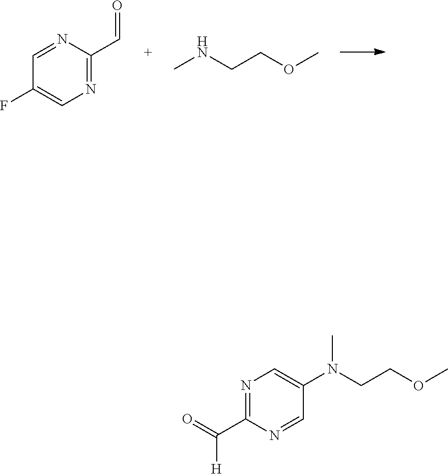

To a stirred solution of 2-methoxy-N-methyl-ethanamine (63 mg, 0.7068 mmol) in anhydrous DMF (1.5 mL) was added 5-fluoropyrimidine-2-carbaldehyde (80 mg, 0.6345 mmol), followed by addition of cesium carbonate (312 mg, 0.9576 mmol). The heterogenous mixture was purged with nitrogen briefly, then stirred at 110° C. for 15 hours (overnight). The mixture was allowed to cool to ambient temperature. The dark reaction mixture was poured over ice-water (15 mL) and extracted with ethyl acetate (2×15 mL). The combined organics were washed sequentially with water (15 mL), brine (15 mL), dried over sodium sulphate, filtered and concentrated under reduced pressure to furnish 5-[2-methoxyethyl(methyl)amino]pyrimidine-2-carbaldehyde (56 mg, 45%) as a tan solid. It was used in the subsequent reaction without further purification. ESI-MS m/z calc. 195.10078, found 196.1 (M+1)⁺; Retention time: 0.83 minutes; (LC method A). ¹H NMR (400 MHz, DMSO-d₆) δ 9.75 (s, 1H), 8.45 (s, 2H), 3.72 (t, J=5.3 Hz, 2H), 3.55 (t, J=5.2 Hz, 2H), 3.25 (s, 3H), 3.10 (s, 3H).

### Example Q: Preparation of 5-[2,2-dimethylpropyl(methyl)amino]pyrimidine-2-carbaldehyde

### Step 1: 5-[2,2-Dimethylpropyl(methyl)amino]pyrimidine-2-carbonitrile

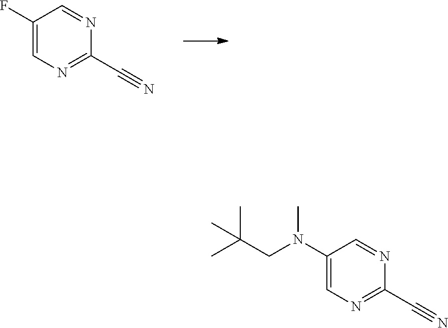

5-Fluoropyrimidine-2-carbonitrile (3.5 g, 27.298 mmol) was dissolved in anhydrous DMF (25 mL) under nitrogen at RT. N,2,2-trimethylpropan-1-amine (hydrochloride salt) (5.1 g, 36.309 mmol) was added to it followed by Cesium carbonate (18.3 g, 56.166 mmol). The reaction mixture was then stirred at 50° C. for 2 h. The reaction mixture was then poured into ice-cold water (200 ml) and stirred for 5 min, and ethyl-acetate (300 ml) was then added to it. The aqueous solution was then extracted with ethyl-acetate (3×300 ml). The combined organic solution was then washed with saturated lithium chloride solution (150 ml) and the organic solution was then dried over anhydrous sodium sulfate, filtered. The solvent was removed by rotary evaporation to furnish 5-[2,2-dimethylpropyl(methyl)amino]pyrimidine-2-carbonitrile (5 g, 81%) as light yellow solid. The product was used in the next step without further purification. ESI-MS m/z calc. 204.1375, found 205.3 (M+1)⁺; Retention time: 4.83 minutes; LC method S.

### Step 2: 5-[2,2-Dimethylpropyl(methyl)amino]pyrimidine-2-carbaldehyde

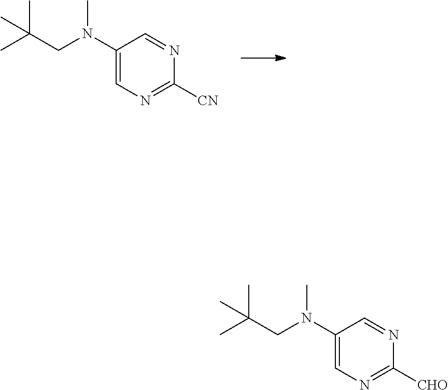

5-[2,2-Dimethylpropyl(methyl)amino]pyrimidine-2-carbonitrile (5.2 g, 24.947 mmol) was dissolved in anhydrous THF (104 mL) under nitrogen and the solution was cooled to −78° C. (acetone & dry-ice bath). After 10 min, diisobutylaluminum hydride in toluene (27.5 mL of 1 M, 27.500 mmol) was added dropwise (over 45 minutes) to the reaction. The reaction was then stirred at the same temperature for another 30 minutes (after addition was complete). Further, Diisobutylaluminum hydride in toluene (5 mL of 1 M, 5.0000 mmol) was added over 15 min and stirred for another 40 minutes. The reaction was then quenched with Methanol (25 mL) at −78° C. and stirred for 15 minutes at the same temperature. After this time reaction mixture was warmed up to RT and 2 M HCl was added and pH was adjusted to pH=3. The organic layer was extracted EtOAc (3×300 mL) and combined organic solution was then washed with brine (200 mL). The organic solution was then dried over anhydrous sodium sulfate, filtered and solvent was removed by rotary evaporation to give crude mixture (weight=12.1 g). The crude product was then purified by flash chromatography (dry loaded in 330 g cartridge and eluting with 0-5% MeOH (3% NH₄₀H) in DCM over 60 min and solvent was removed to provide 5-[2,2-dimethylpropyl(methyl)amino]pyrimidine-2-carbaldehyde (2.72 g, 51%) as light yellow. ESI-MS m/z calc. 207.13716, found 208.0 (M+1)⁺; Retention time: 1.73 minutes; LC method W. ¹H NMR (500 MHz, DMSO-d₆) δ 9.74 (s, 1H), 8.51 (s, 2H), 3.41 (s, 2H), 3.14 (s, 3H), 0.95 (s, 9H).

### Example R: Preparation of 5-isopropoxypyrimidine-2-carbaldehyde 1. Step 1: 5-Hydroxypyrimidine-2-carbonitrile

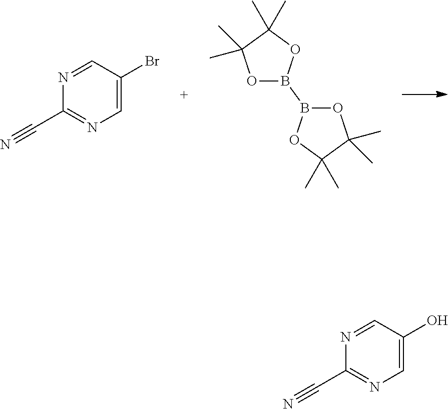

Into a reaction flask was charged with 5-bromopyrimidine-2-carbonitrile (10 g, 54.350 mmol), 4,4,5,5-tetramethyl-2-(4,4,5,5-tetramethyl-1,3,2-dioxaborolan-2-yl)-1,3,2-dioxaborolane (20.75 g, 81.713 mmol) and Potassium acetate (16.1 g, 164.05 mmol) in anhydrous 1,4-dioxane (100 mL). The reaction was purged with nitrogen for 10 minutes. Under nitrogen Pd(dppf)Cl₂ (2 g, 2.7333 mmol) was added. The reaction was purged with nitrogen for another 5 minutes. The reaction was then heated at 80° C. and stirred for 3 hours. The reaction was cooled to rt and diluted with 10% ammonium chloride aqueous solution (100 mL). The reaction was acidified to pH 1 with 3 N aqueous HCl. The reaction was extracted with ethyl acetate (5×100 mL). The combined organic layers were washed with brine (100 mL), dried over anhydrous sodium sulfate and concentrated under vacuum. The crude boronic ester was dissolved in THF (100 mL) and water (100 mL). sodium perborate tetrahydrate (47.6 g, 309.37 mmol) was added to the reaction mixture. The reaction was stirred at rt overnight. The volatiles were removed under vacuum. The pH was adjusted to 10 with 15% aq. NaOH. The solid was removed by filtration. The filtrate was washed with ether (3×100 mL). Ether layers were discarded. The aqueous layer was acidified with concentrated HCl to pH 1 and was extracted with ethyl acetate (5×100 mL). The combined organic layers were washed with brine (100 mL), dried over anhydrous sodium sulfate and concentrated under vacuum. The residue was purified by silica gel chromatography using 0-5% MeOH in DCM to furnish the crude product (9.23 g). It was further purified by HPLC (Buffer A: water buffered with 5 mM HCl; Buffer B: ACN, eluting from 10 to 50% in 40 min). The fractions containing the product were combined and rotary evaporated to remove ACN. The pH was then adjusted to 4 using conc. HCl and the aqueous layer was extracted with 3:1 chloroform/isopropanol (3×100 mL). The combined organic solutions were dried over anhydrous sodium sulfate, filtered, and rotary evaporated. The residue was further dried in vacuo overnight yielding 5-hydroxypyrimidine-2-carbonitrile (2.7 g, 41%) as a white solid. ¹H NMR (500 MHz, DMSO) δ 11.81 (s, 1H), 8.45 (s, 2H). ESI-MS m/z calc. 121.02761, no ionization, Retention time: 1.43 minutes; LC method T.

### Step 2: 5-Isopropoxypyrimidine-2-carbonitrile

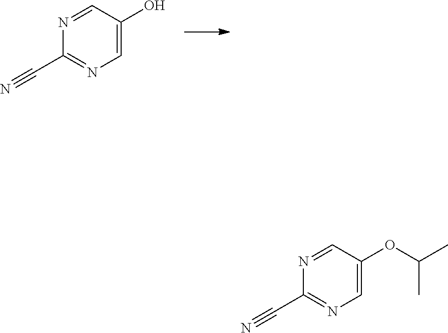

A solution of propan-2-ol (3.5325 g, 4.5 mL, 58.782 mmol), triphenylphosphine (5.8 g, 22.113 mmol), and 5-hydroxypyrimidine-2-carbonitrile (3.12 g, 15.459 mmol) in DCM (20 mL) was added with a solution of Bis(4-chlorobenzyl) azodicarboxylate (DCAD, 7.55 g, 20.562 mmol) in DCM (60 mL) dropwise at rt. The resulting orange solution was stirred at rt overnight. The reaction mixture was filtered, and the filtrate was concentrated. The residue was dry loaded onto silica and purified by flash chromatography (220 g column, 0 to 20% EtOAc in hexanes) yielding 5-isopropoxypyrimidine-2-carbonitrile (2.59 g, 100%) as a white solid. ¹H NMR (500 MHz, DMSO) δ 8.72 (s, 2H), 4.96 (hept, J=6.0 Hz, 1H), 1.33 (d, J=6.0 Hz, 6H). ESI-MS m/z calc. 163.07455, Retention time: 1.79 minutes; LC method W.

### Step 3: 5-Isopropoxypyrimidine-2-carbaldehyde

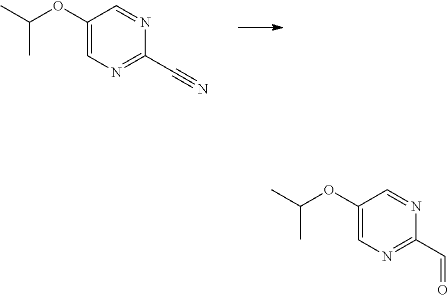

5-Isopropoxypyrimidine-2-carbonitrile (0.5 g, 2.9722 mmol) was dissolved in anhydrous THF (14 mL) under nitrogen and was cooled to −78° C. using an acetone-dry ice bath (stirred at this temperature for 30 minutes). DIBAL in toluene (3.6 mL of 1 M, 3.6000 mmol) was added dropwise over 15 minutes to the reaction mixture. The reaction was then stirred at the same temperature for 1 hours. LCMS showed some SM left so more DIBAL in toluene (0.6 mL of 1 M, 0.6000 mmol) was added and stirring was continued at the same temperature for 1 hour. The reaction was then quenched with saturated aq. sodium potassium tartrate solution (10 mL) and stirred for 15 minutes. More Rochelle's salt solution (100 mL) and EtOAc (100 mL) were added and the reaction mixture was stirred and allowed to warm up to RT over 2 hours. Layers were separated, and the aqueous layer was further extracted with EtOAc (2×100 ml). The combined organic solutions were then washed with brine (150 ml), dried over anhydrous sodium sulfate, filtered, and concentrated. The residue was dried in vacuo for 30 min. The crude 5-isopropoxypyrimidine-2-carbaldehyde (0.56 g, 100%) (brown oil) was used in the next step without further purification. ESI-MS m/z calc. 166.07423, found 166.9 (M+1)⁺; Retention time: 1.77 minutes; LC method T.

### Example S: Preparation of (2R)-4,4-dimethyl-2-(spiro[2.3]hexan-5-ylamino)pentan-1-ol

### Step 1: (2R)-4,4-Dimethyl-2-(spiro[2.3]hexan-5-ylamino)pentan-1-ol

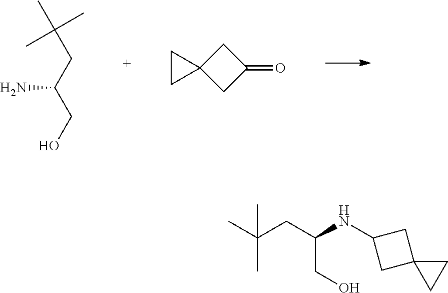

(2R)-2-Amino-4,4-dimethyl-pentan-1-ol (hydrochloride salt) (504 mg, 3.0058 mmol) was dissolved in DCE (10 mL) and spiro[2.3]hexan-5-one (321 mg, 3.3393 mmol) was added. The mixture was stirred at room temperature for 30 minutes, then sodium triacetoxyborohydride (1.92 g, 9.0591 mmol) was added in 3 batches. The mixture was allowed to stir overnight and then 1 M HCl was added until a pH of ˜1 was achieved. The mixture was stirred vigorously for 20 minutes. The layers were separated, and the aqueous layer was extracted twice with DCM (10 mL). The organic layers were dried over sodium sulfate and concentrated to afford (2R)-4,4-dimethyl-2-(spiro[2.3]hexan-5-ylamino)pentan-1-ol (570 mg, 90%) as a viscous colorless oil. ¹H NMR (250 MHz, DMSO) δ 4.51 (t, J=5.5 Hz, 1H), 3.53-3.41 (m, 1H), 3.35-3.25 (m, 1H), 3.19-3.03 (m, 1H), 2.18-2.01 (m, 2H), 2.02-1.85 (m, 2H), 1.14 (d, J=4.8 Hz, 2H), 0.89 (s, 9H), 0.47-0.27 (m, 4H).

### Example T: Preparation of 2-(methylamino)-1-[5-[[1-(trifluoromethyl)cyclopropyl]methyl]-2-pyridyl]ethanol

### Step 1: N-Methoxy-N-methyl-1-(trifluoromethyl)cyclopropanecarboxamide

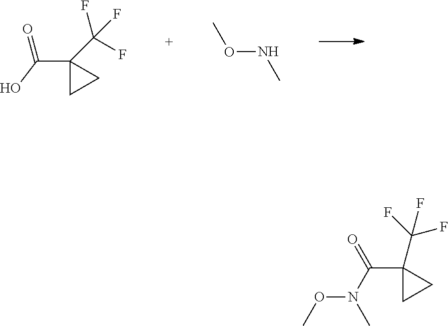

1,1′-Carbonyldiimidazole (14.0 g, 86.340 mmol) was added portionwise to a solution of 1-(trifluoromethyl)cyclopropanecarboxylic acid (10 g, 64.898 mmol) in dichloromethane (100 mL) at 15° C. in 15 min. The mixture was stirred at 20° C. for 2 hours. Triethylamine (9.2 g, 12.672 mL, 90.918 mmol), then N-methoxymethanamine (hydrochloride salt) (8.9 g, 91.241 mmol) were added and the mixture was stirred at room temperature 18 hours. A hydrochloric acid solution (3 N, 65 mL) was added at 5° C. and phases were separated. The organic layer was washed with a potassium bicarbonate solution (10% w/w, 20 mL), dried over sodium sulfate and concentrated under reduced pressure to afford N-methoxy-N-methyl-1-(trifluoromethyl)cyclopropanecarboxamide (11.87 g, 93%). ¹H NMR (400 MHz, CDCl₃) δ 3.74 (s, 3H), 3.28 (s, 3H), 1.36-1.17 (m, 4H).

### Step 2: (6-Bromo-3-pyridyl)-[1-(trifluoromethyl)cyclopropyl]methanone

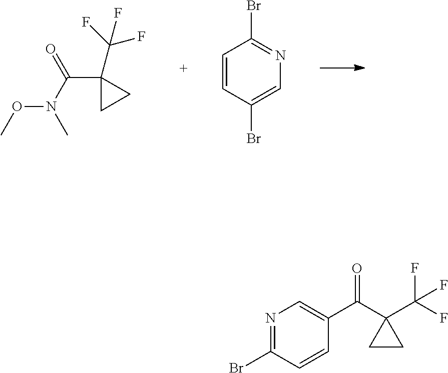 [SMILES: CON(C)C(=O)C1(C(F)(F)F)CC1 ; Brc1ccc(Br)nc1 ; O=C(c1ccc(Br)nc1)C1(C(F)(F)F)CC1]

2,5-Dibromopyridine (57 g, 240.62 mmol) was dissolved in diethyl ether (1000 mL) then cooled to −78° C. Then n-BuLi 2.5M in hexanes (120 mL of 2.5 M, 300.00 mmol) was added drop wise via addition funnel keeping the internal reaction temperature below −70° C. The mixture was stirred for an hour, then N-methoxy-N-methyl-1-(trifluoromethyl)cyclopropanecarboxamide (59.45 g, 301.54 mmol) in diethyl ether (150 mL) was added to the mixture via addition funnel and the reaction was stirred at −78° C. for 1 hour. The reaction was allowed to warm to −10° C., then quenched by the addition of NH₄Cl solution (500 mL). The phases were separated, and the aqueous layer was extracted with diethyl ether (200 mL). The organic layer was washed with water and brine (200 mL each) and then dried over sodium sulfate and concentrated. The crude residue was combined with another batch of crude product from another reaction run on 61 g scale and loaded on to silica gel and purified by flash column chromatography (220 g+330 g column) using 0-20% EtOAc in hexanes. The appropriate fractions were collected to give (6-bromo-3-pyridyl)-[1-(trifluoromethyl)cyclopropyl]methanone (86.41 g, 85% corrected yield). ESI-MS m/z calc. 292.9663, found 294.2 (M+1)⁺; Retention time: 3.17 minutes; LC method T.

### Step 3: (6-Bromo-3-pyridyl)-[1-(trifluoromethyl)cyclopropyl]methanol

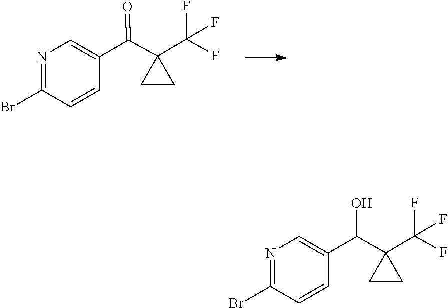 [SMILES: O=C(c1ccc(Br)nc1)C1(C(F)(F)F)CC1 ; OC(c1ccc(Br)nc1)C1(C(F)(F)F)CC1]

(6-Bromo-3-pyridyl)-[1-(trifluoromethyl)cyclopropyl]methanone (86.41 g, 293.84 mmol) was dissolved in EtOH (1000 mL) then sodium borohydride (12.46 g, 329.35 mmol) was added portion wise at room temperature. The reaction was allowed to stir for 1.5 hours, then quenched with acetone (750 mL). The volatiles were removed under vacuum and the crude residue was taken up in EtOAc (700 mL) and washed with water and brine (400 mL each). The organic layer was dried over sodium sulfate and concentrated. The crude residue was loaded on to silica gel and purified by flash column chromatography using 0-30% EtOAc in hexanes to give (6-bromo-3-pyridyl)-[1-(trifluoromethyl)cyclopropyl]methanol (74.38 g, 51%) as a colorless oil. ESI-MS m/z calc. 294.98196, found 296.2 (M+1)⁺; Retention time: 2.7 minutes; LC method T.

### Step 4: [(6-Bromo-3-pyridyl)-[1-(trifluoromethyl)cyclopropyl]methyl]methanesulfonate

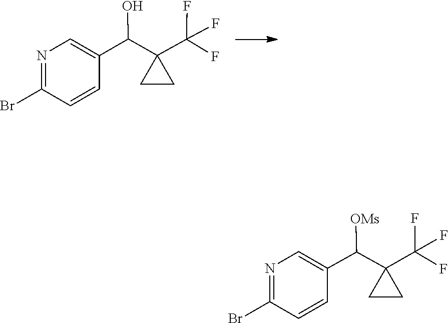 [SMILES: OC(c1ccc(Br)nc1)C1(C(F)(F)F)CC1 ; CS(=O)(=O)OC(c1ccc(Br)nc1)C1(C(F)(F)F)CC1]

(6-Bromo-3-pyridyl)-[1-(trifluoromethyl)cyclopropyl]methanol (74.38 g, 200.97 mmol) was dissolved in Pyridine (225 mL) then cooled to 0° C. MsCl (28.860 g, 19.5 mL, 251.94 mmol) was added via syringe and the ice bath was removed and the reaction stirred for 3.5 hours at room temperature. After 4.5 hours the reaction was quenched by the addition of 1 L EtOAc, then 600 mL water. The layers were separated, and the organic layer washed with 1M HCl (600 mL), sat. sodium bicarbonate solution (600 mL) and brine (4×150 mL). The organic layer was dried over sodium sulfate and concentrated. The crude residue was dry loaded on to silica gel and purified by flash column chromatography using 0-30% EtOAc in hexanes. The purification conditions were unsuccessful in giving satisfactorily pure material, even after splitting into smaller batches. The crude material was loaded on to silica gel again and purified using 1-5% acetone in DCM to give good separation and the appropriate fractions were collected to give [(6-bromo-3-pyridyl)-[1-(trifluoromethyl)cyclopropyl]methyl]methanesulfonate (35.34 g, 45%) as colorless crystals. ¹H NMR (500 MHz, DMSO-d₆) δ 8.52 (d, J=2.5 Hz, 1H), 7.86 (dd, J=8.3, 2.6 Hz, 1H), 7.74 (d, J=8.3 Hz, 1H), 5.75 (s, 1H), 3.23 (s, 3H), 1.55-1.47 (m, 1H), 1.26 (dt, J=9.9, 6.1 Hz, 1H), 1.17 (ddd, J=9.9, 7.0, 5.5 Hz, 1H), 1.01 (qt, J=6.9, 5.2, 2.3 Hz, 1H). ESI-MS m/z calc. 372.9595, found 374.1 (M+1)⁺; Retention time: 3.04 minutes; LC method T.

### Step 5: 2-Bromo-5-[[1-(trifluoromethyl)cyclopropyl]methyl]pyridine

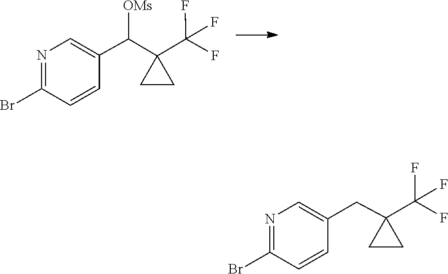 [SMILES: CS(=O)(=O)OC(c1ccc(Br)nc1)C1(C(F)(F)F)CC1 ; FC(F)(F)C1(Cc2ccc(Br)nc2)CC1]

The reaction was run in 7 separate batches using each 1.5 g of [(6-bromo-3-pyridyl)-[1-(trifluoromethyl)cyclopropyl]methyl] methanesulfonate and 1 batch using 1.17 g of [(6-bromo-3-pyridyl)-[1-(trifluoromethyl)cyclopropyl]methyl] methanesulfonate. DMF (20 mL) was added to a heat dried 100 mL flask under nitrogen, and purged with nitrogen gas for 20 min. [(6-Bromo-3-pyridyl)-[1-(trifluoromethyl)cyclopropyl]methyl] methanesulfonate (1.5 g) was added followed by sodium borohydride (520 mg) and the mixture was placed in a pre-heated oil bath at 100° C. for exactly 20 minutes. The mixture was cooled rapidly in a water bath, then diluted with water (100 mL) and combined with the other batches of the quenched material. The aqueous layer was extracted three times with EtOAc (200 mL each). The organic layers were washed with water (200 mL) and brine (3×150 mL), dried over sodium sulfate and concentrated. The crude residue was dry loaded on to silica gel and purified using flash column chromatography using 0-30% EtOAc in hexanes as an eluent. The appropriate fractions were collected to give 2-bromo-5-[[1-(trifluoromethyl)cyclopropyl]methyl]pyridine (2.6103 g, 28%) as a colorless oil. ¹H NMR (250 MHz, DMSO-d₆) δ 8.32 (d, J=2.5 Hz, 1H), 7.71 (dd, J=8.2, 2.5 Hz, 1H), 7.61 (d, J=8.2 Hz, 1H), 2.94 (s, 2H), 1.06-0.85 (m, 4H).

### Step 6: tert-Butyl N-[2-[methoxy(methyl)amino]-2-oxo-ethyl]-N-methyl-carbamate

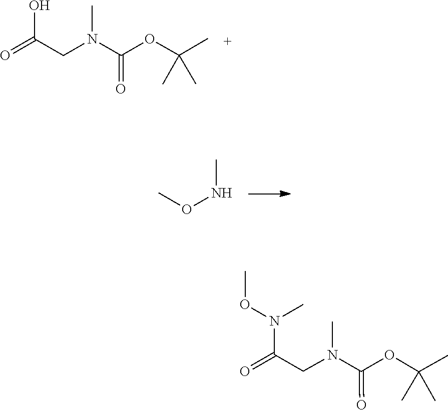

In a 1-L round-bottomed flask, 2-[tert-butoxycarbonyl(methyl)amino]acetic acid (25.85 g, 136.6 mmol), DCM (200 mL), DMF (200 mL), N-methoxymethanamine (hydrochloride salt) (17.10 g, 175.3 mmol), DIPEA (120 mL, 688.9 mmol), HOBt (24.22 g, 179.2 mmol) and EDCI (hydrochloride salt)(40 g, 175.3 mmol) were added in this order. This solution was stirred at room temperature for 3.5 h, after which it was diluted with ethyl acetate (1 L). This mixture was washed with 1N HCl solution (2×500 mL), 1N NaOH solution (2×500 mL), water (500 mL) and saturated aqueous sodium chloride solution (500 mL), then dried over sodium sulfate, filtered, and evaporated in vacuo to give a colorless liquid, tert-butyl N-[2-[methoxy(methyl)amino]-2-oxo-ethyl]-N-methyl-carbamate (25.1688 g, 79%)¹H NMR (400 MHz, dimethylsulfoxide-d6, mixture of 2 distinct rotamers) δ 4.08 and 4.07 (two singlets, 2H), 3.68 and 3.66 (two singlets, 3H), 3.10 and 3.09 (two singlets, 3H), 2.82 and 2.78 (two singlets, 3H), 1.39 and 1.33 (two singlets, 9H). ESI-MS m/z calc. 232.1423, found 233.0 (M+1)⁺; Retention time: 1.02 minutes; LC method A.

### Step 7: tert-Butyl N-methyl-N-[2-oxo-2-[5-[[1-(trifluoromethyl)cyclopropyl]methyl]-2-pyridyl]ethyl]carbamate

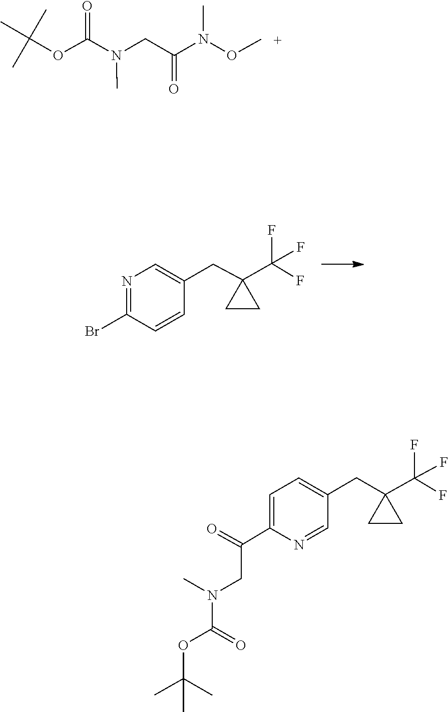 [SMILES: CON(C)C(=O)CN(C)C(=O)OC(C)(C)C ; FC(F)(F)C1(Cc2ccc(Br)nc2)CC1 ; CN(CC(=O)c1ccc(CC2(C(F)(F)F)CC2)cn1)C(=O)OC(C)(C)C]

n-BuLi (12.6 mL of 2.5 M, 31.500 mmol) was added dropwise to a suspension of 2-bromo-5-[[1-(trifluoromethyl)cyclopropyl]methyl]pyridine (8 g, 28.563 mmol) in diethyl ether (110 mL) cooled at −78° C. The reaction was stirred for one hour at the same temperature, during which time it slowly turned to a red solution. A solution of tert-butyl N-[2-[methoxy(methyl)amino]-2-oxo-ethyl]-N-methyl-carbamate (9.2 g, 39.608 mmol) in diethyl ether (24 mL) was added dropwise. The obtained red solution was stirred at −78° C. for one hour, and then allowed to slowly warm up to −10° C. over one hour. The reaction was quenched with water (100 mL) and warmed up to rt. EtOAc (200 mL) was added. The two layers were separated, and the aqueous layer was extracted with EtOAc (200 mL). The combined organic layers were washed with brine (40 mL), dried over sodium sulfate and concentrated to provide crude product. The resulting crude was purified by companion (220 g, eluting 0 to 30% ethyl acetate/heptanes) to give tert-butyl N-methyl-N-[2-oxo-2-[5-[[1-(trifluoromethyl)cyclopropyl]methyl]-2-pyridyl]ethyl]carbamate (5.75 g, 54%) as yellow solid. ESI-MS m/z calc. 372.16608, found 373.2 (M+1)⁺; Retention time: 2.15 minutes; LC method K.

### Step 8: tert-Butyl N-[2-hydroxy-2-[5-[[1-(trifluoromethyl)cyclopropyl]methyl]-2-pyridyl]ethyl]-N-methyl-carbamate

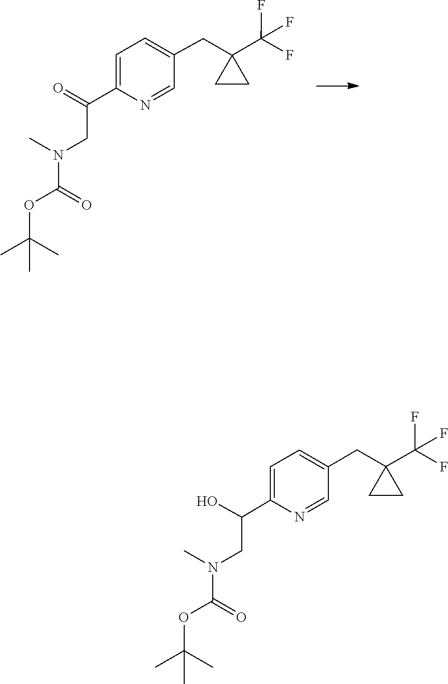 [SMILES: CN(CC(=O)c1ccc(CC2(C(F)(F)F)CC2)cn1)C(=O)OC(C)(C)C ; CN(CC(O)c1ccc(CC2(C(F)(F)F)CC2)cn1)C(=O)OC(C)(C)C]

Sodium borohydride (2.69 g, 71.103 mmol) was added to a solution of tert-butyl N-methyl-N-[2-oxo-2-[5-[[1-(trifluoromethyl)cyclopropyl]methyl]-2-pyridyl]ethyl]carbamate (14.3 g, 38.401 mmol) in MeOH (88 mL) and THF (22 mL) cooled with ice-water. The reaction was stirred at 0° C. for 20 minutes. LCMS showed complete. The reaction was quenched with water (100 mL). The mixture was concentrated at 30° C. to remove most of organic solvents. The residue was extracted with EtOAc (200 mL×2). The combined organic extracts were washed with brine (40 mL), dried over sodium sulfate, filtered and concentrated under reduced pressure. The crude product was purified by flash chromatography (dry-loading) (220 g silica gel, eluting 10 to 60% EtOAc/heptanes), product fractions were combined and concentrated in vacuo to afford tert-butyl N-[2-hydroxy-2-[5-[[1-(trifluoromethyl)cyclopropyl]methyl]-2-pyridyl]ethyl]-N-methyl-carbamate (13.7 g, 95%) as light yellow oil. ESI-MS m/z calc. 374.18173, found 375.2 (M+1)⁺; Retention time: 1.81 minutes; LC method K.

### Step 9: 2-(Methylamino)-1-[5-[[1-(trifluoromethyl)cyclopropyl]methyl]-2-pyridyl]ethanol

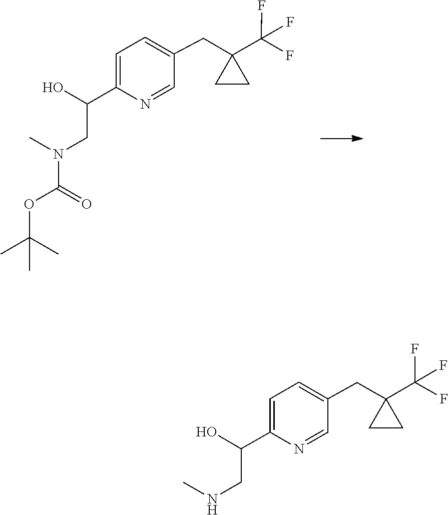 [SMILES: CN(CC(O)c1ccc(CC2(C(F)(F)F)CC2)cn1)C(=O)OC(C)(C)C ; CNCC(O)c1ccc(CC2(C(F)(F)F)CC2)cn1]

HCl (137 mL of 4 M, 548.00 mmol) in 1,4-dioxane was added to a solution of tert-butyl N-[2-hydroxy-2-[5-[[1-(trifluoromethyl)cyclopropyl]methyl]-2-pyridyl]ethyl]-N-methyl-carbamate (13.7 g, 36.592 mmol) in 1,4-dioxane (300 mL) at rt. After the addition, the mixture was stirred at rt overnight. A white suspension was obtained. The reaction was concentrated to dryness. The solid was triturated in a mixture of DCM (100 mL) and heptanes (200 mL). The obtained solid was filtered off and dried under high vacuum to afford 2-(methylamino)-1-[5-[[1-(trifluoromethyl)cyclopropyl]methyl]-2-pyridyl]ethanol (Hydrochloric acid (2)) (11.7 g, 91%) as white solid. ¹H NMR (300 MHz, DMSO-d₆) δ 9.19 (br. s., 1H), 8.97 (br. s., 1H), 8.59 (d, J=1.8 Hz, 1H), 8.11 (d, J=6.8 Hz, 1H), 7.76 (d, J=8.2 Hz, 1H), 5.17 (dd, J=9.4, 2.9 Hz, 1H), 3.45-3.30 (m, 1H), 3.23-3.08 (m, 1H), 3.05 (s, 2H), 2.60 (t, J=5.4 Hz, 3H), 1.05-0.96 (m, 2H), 0.95-0.86 (m, 2H). ¹⁹F NMR (282 MHz, DMSO-d₆) δ −67.50 (s, 3F). ESI-MS m/z calc. 274.1293, found 275.2 (M+1)⁺; Retention time: 1.75 minutes; LC method G.

### Example U: Preparation of (2R)-2-[[6-(1-Hydroxy-1-methyl-ethyl)spiro[3.3]heptan-2-yl]amino]-4,4-dimethyl-pentan-1-ol

### Step 1: Methyl 2-[[(1R)-1-(hydroxymethyl)-3,3-dimethyl-butyl]amino]spiro[3.3]heptane-6-carboxylate

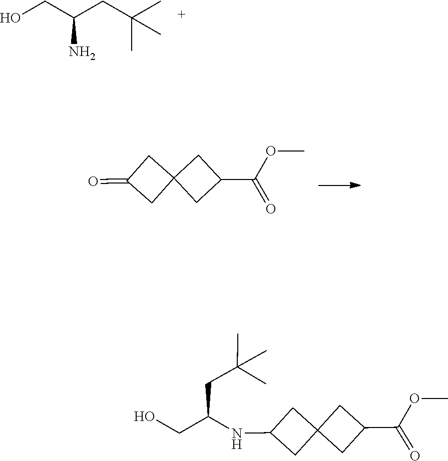

A 500 mL round bottom flask equipped with a magnetic stirbar was charged under nitrogen with methyl 2-oxospiro[3.3]heptane-6-carboxylate (12.18 g, 72.42 mmol) and anhydrous DCE (200 mL). Stirring was commenced and (2R)-2-amino-4,4-dimethyl-pentan-1-ol (hydrochloride salt) (12.2 g, 72.76 mmol) was added. To the suspension, DIEA (15 mL, 86.12 mmol) and acetic acid (4.8 mL, 84.41 mmol) were added and the reaction was stirred at rt for 5-10 min (complete dissolution). sodium triacetoxyborohydride (22.5 g, 106.2 mmol) was added and stirring was continued at rt for 16 h. The reaction was cooled down in an ice-bath (internal temperature 2° C.) and was quenched by slow addition of aqueous HCl (40 mL of 4 M, 160.0 mmol) while maintaining temperature below 7° C. A suspension of sodium bicarbonate (45 g, 535.7 mmol) in water (100 mL) was slowly added (foaming) while maintaining temperature below 10° C. More water (50 mL) and brine (50 mL) were added and the mixture was stirred until gas evolution stopped (final pH=7-8). The two phases were separated, and the aqueous phase was further extracted with DCM (3×50 mL). The combined extracts were washed with brine (50 mL), dried over sodium sulfate and filtered through a pad of Celite. Evaporation of the solvents gave methyl 2-[[(1R)-1-(hydroxymethyl)-3,3-dimethyl-butyl]amino]spiro[3.3]heptane-6-carboxylate (21.08 g, 100%) as a white solid. ESI-MS m/z calc. 283.21475, found 284.12 (M+1)⁺; Retention time: 0.88 minutes; LC method A. ¹H NMR (400 MHz, DMSO-d₆) δ 7.77 (broad s, 1H), 5.23 (broad s, 1H), 3.57 (s, 3H), 3.54-3.42 (m, 2H), 3.31 (dd, J=11.8, 5.9 Hz, 1H), 3.09-2.97 (m, 1H), 2.73 (br s, 1H), 2.40-1.95 (m, 8H), 1.39 (dd, J=14.4, 7.6 Hz, 1H), 1.25 (dd, J=14.4, 2.4 Hz, 1H), 0.89 (s, 9H).

### Step 2: (2R)-2-[[6-(1-Hydroxy-1-methyl-ethyl)spiro[3.3]heptan-2-yl]amino]-4,4-dimethyl-pentan-1-ol

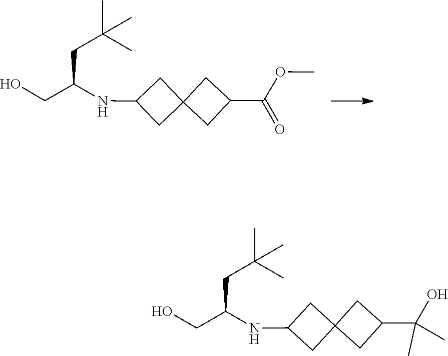 [SMILES: COC(=O)C1CC2(CC(N[C@@H](CO)CC(C)(C)C)C2)C1 ; CC(C)(C)C[C@H](CO)NC1CC2(C1)CC(C(C)(C)O)C2]

A 100 mL flask was charged under nitrogen with methyl 2-[[(1R)-1-(hydroxymethyl)-3,3-dimethyl-butyl]amino]spiro[3.3]heptane-6-carboxylate (322 mg, 1.136 mmol) and anhydrous THF (7 mL). The mixture was cooled down in an ice bath and MeMgBr (1.7 mL of 3 M, 5.100 mmol) (3 M in diethyl ether) was added dropwise through syringe over a period of 5 minutes (gas evolution visible at the beginning of the addition). At the end of the addition (clear solution), the ice bath was removed, and the reaction was stirred at room temperature for 1 hour. More THF (4 mL) was added to ease stirring. After 30 min, more MeMgBr (0.2 mL of 3 M, 0.6000 mmol) was added and the mixture was stirred at room temperature for 6 hours. The reaction was cooled down and treated with saturated ammonium chloride (50 mL), brine (30 mL) and EtOAc (40 mL). The two phases were separated, and the aqueous phase was extracted with EtOAc (3×30 mL). The combined extracts were washed with brine (40 mL) dried over sodium sulfate and the solvents evaporated to give crude (2R)-2-[[6-(1-hydroxy-1-methyl-ethyl)spiro[3.3]heptan-2-yl]amino]-4,4-dimethyl-pentan-1-ol (288 mg, 89%) as a colorless resin. ESI-MS m/z calc. 283.25113, found 284.18 (M+1)⁺; Retention time: 0.91 minutes; LC method A.

### Example V: Preparation of (2R)-2-(Spiro[2.3]hexan-5-ylamino)-3-[1-(trifluoromethyl)cyclopropyl]propan-1-ol

### Step 1: (2R)-2-(Spiro[2.3]hexan-5-ylamino)-3-[1-(trifluoromethyl)cyclopropyl]propan-1-ol

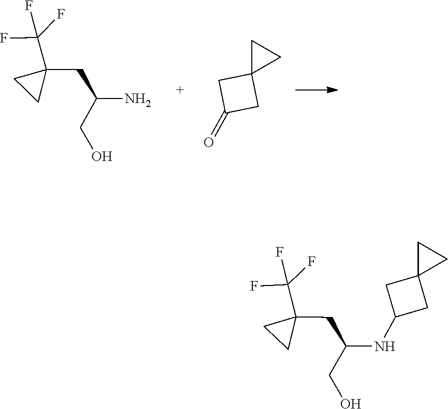 [SMILES: N[C@@H](CO)CC1(C(F)(F)F)CC1 ; O=C1CC2(CC2)C1 ; OC[C@@H](CC1(C(F)(F)F)CC1)NC1CC2(CC2)C1]

To a stirred milky emulsion of (2R)-2-amino-3-[1-(trifluoromethyl)cyclopropyl]propan-1-ol (hydrochloride salt) (600 mg, 2.732 mmol) in anhydrous 1,2-dichloroethane (15 mL) was added a solution of spiro[2.3]hexan-5-one (330 mg, 3.433 mmol) in anhydrous 1,2-dichloroethane (3 mL), followed by addition of glacial acetic acid (200 μL, 3.517 mmol), at ambient temperature under nitrogen. The mixture was stirred for 30 minutes, then solid sodium triacetoxyborohydride (2.22 g, 10.47 mmol) was added in 3 batches (equal amounts) in 2 minutes intervals. The reaction was stirred overnight (13 h). The suspension was cooled in an ice-water bath and quenched by slow addition of aqueous hydrochloric acid (6 mL of 2.0 M, 12.00 mmol) to pH about 1.0. The emulsion (no clear phases) was stirred for 20 minutes. The resulting cooled (ice-bath) suspension was basified by slow addition of sodium carbonate (1.50 g, 14.15 mmol) (Caution! strong effervescence) to adjust pH about 10. The heterogeneous phases were separated, and the aqueous layer was extracted with methylene chloride (2×40 mL). The combined organics were washed with brine (20 mL), dried over sodium sulfate, filtered, and evaporated under reduced pressure to give (2R)-2-(spiro[2.3]hexan-5-ylamino)-3-[1-(trifluoromethyl)cyclopropyl]propan-1-ol (698 mg, 97%) as clear thick oil, which became solid overnight. ESI-MS m/z calc. 263.1497, found 264.1 (M+1)⁺; Retention time: 0.91 minutes; LC method A, ¹H NMR (500 MHz, DMSO-d₆) δ 3.48 (p, J=7.4 Hz, 1H), 3.39 (dd, J=10.6, 4.6 Hz, 1H), 3.28 (dd, J=10.8, 5.2 Hz, 1H), 2.66-2.58 (m, 1H), 2.24-2.04 (m, 2H), 2.00-1.81 (m, 2H), 1.78-1.61 (m, 1H), 1.52 (dd, J=14.9, 7.1 Hz, 1H), 1.00-0.72 (m, 4H), 0.41 (dd, J=9.0, 6.2 Hz, 2H), 0.33 (dd, J=8.8, 5.9 Hz, 2H).

### Example W: Preparation of tert-butyl 2-[[(1R)-1-(hydroxymethyl)-3,3-dimethyl-butyl]amino]-7-azaspiro[3.5]nonane-7-carboxylate

### Step 1: tert-Butyl 2-[[(1R)-1-(hydroxymethyl)-3,3-dimethyl-butyl]amino]-7-azaspiro[3.5]nonane-7-carboxylate

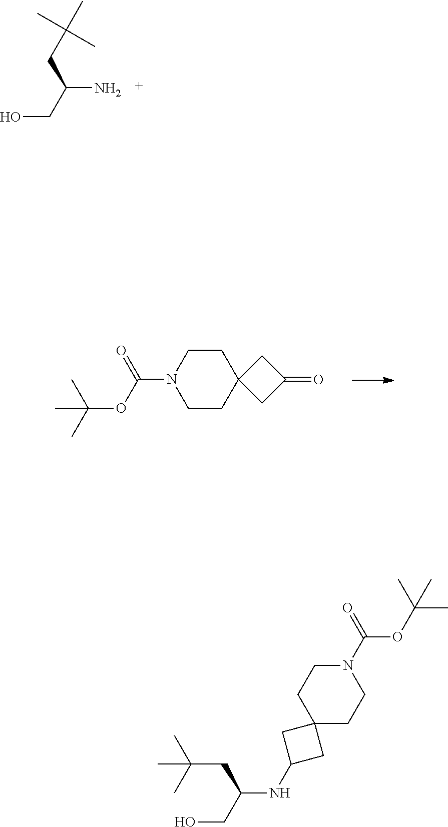

In a 25 mL flask, to a stirred milky emulsion of (2R)-2-amino-4,4-dimethyl-pentan-1-ol (hydrochloride salt) (200 mg, 1.193 mmol) in anhydrous 1,2-dichloroethane (3 mL) was added solid tert-butyl 2-oxo-7-azaspiro[3.5]nonane-7-carboxylate (370 mg, 1.546 mmol) at ambient temperature under nitrogen. The mixture was stirred for 20 minutes, then solid sodium triacetoxyborohydride (850 mg, 4.011 mmol) was added in 3 batches in 2 minute intervals. The reaction was stirred overnight (14 hours). The suspension was cooled in an ice-water bath and quenched by slow addition of aqueous hydrochloric acid (6.0 mL of 1.0 M, 6.000 mmol) to adjust pH to about 1. The emulsion was stirred for 20 min to break-up the emulsion. The resulting cooled (ice-bath) suspension was basified by slow addition of solid sodium carbonate (1.5 g, 14.15 mmol) (Caution! strong effervescence) to adjust pH to 10. The heterogeneous phases were stirred for 20 minutes. Then ethyl acetate (20 mL) was added and the layers were separated. The aqueous layer was extracted back with ethyl acetate (2×10 mL). The combined organics were washed with brine (10 mL), dried over sodium sulfate, filtered, and evaporated under reduced pressure to give tert-butyl 2-[[(1R)-1-(hydroxymethyl)-3,3-dimethyl-butyl]amino]-7-azaspiro[3.5]nonane-7-carboxylate (499 mg, 98%) as colorless viscous material. It was used in the subsequent reaction without further purification. ESI-MS m/z calc. 354.28824, found 355.4 (M+1)⁺; Retention time: 0.99 minutes; LC method A.

### Example X: Preparation of (2R)-2-[(3-benzyloxycyclobutyl)amino]-4,4-dimethyl-pentan-1-ol

### Step 1: (2R)-2-[(3-Benzyloxycyclobutyl)amino]-4,4-dimethyl-pentan-1-ol

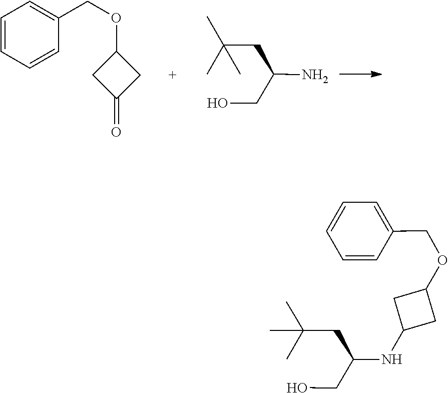

Into a solution of 3-benzyloxycyclobutanone (3.014 g, 16.591 mmol) in anhydrous DCE (30 mL) was added (2R)-2-amino-4,4-dimethyl-pentan-1-ol (hydrochloride salt) (3.527 g, 21.035 mmol). The reaction was stirred at rt for 30 minutes, before sodium triacetoxyborohydride (6.496 g, 29.118 mmol) was added. The reaction mixture was stirred at rt for 2 hours. The reaction was quenched with 2 N sodium carbonate (aqueous) (30 mL). The aqueous layer was extracted with chloroform (3×30 mL). The combined organic layers were washed with brine (20 mL), dried over anhydrous sodium sulfate and concentrated under vacuum. The residue was purified by silica gel chromatography using 0 to 10% methanol in dichloromethane (buffered with 0.3% NH4OH) to furnish (2R)-2-[(3-benzyloxycyclobutyl)amino]-4,4-dimethyl-pentan-1-ol (4.905 g, 100%) as a clear gel. The product is a mixture of diastereomers. ESI-MS m/z calc. 291.21982, found 292.3 (M+1)⁺; Retention time: 2.29 minutes; LC method T.

### Example Y: Preparation of 7,10-dioxadispiro[3.1.46.14]undecan-2-one

### Step 1: Methyl 7,10-dioxadispiro[3.1.46.14]undecane-2-carboxylate

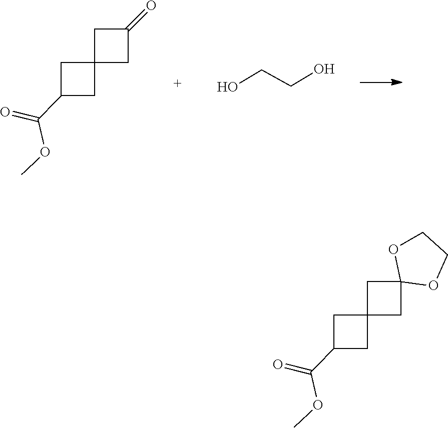

To a stirring solution of methyl 2-oxospiro[3.3]heptane-6-carboxylate (19.663 g, 116.91 mmol) and ethylene glycol (15.582 g, 14 mL, 251.05 mmol) in toluene (190 mL) at room temperature under ambient conditions was added p-toluenesulfonic acid hydrate (1.141 g, 5.9984 mmol). The reaction mixture was heated to reflux (140° C.) with Dean-Stark apparatus for 24 hours. After cooling to room temperature, the reaction mixture was quenched with saturated aqueous sodium bicarbonate (350 mL). Two layers were separated, and the aqueous layer was extracted with ethyl acetate (2×300 mL). The combined organic layers were washed with brine (150 mL), dried over anhydrous sodium sulfate and concentrated to afford methyl 7,10-dioxadispiro[3.1.46.14]undecane-2-carboxylate (27.67 g, 100%) as pale-yellow oil. The product was carried to the next step without further purification. ¹H NMR (250 MHz, CDCl₃) δ 4.34-4.13 (m, 2H), 3.91-3.79 (m, 5H), 3.15-2.93 (m, 1H), 2.49-2.37 (m, 4H), 2.35-2.26 (m, 4H).

### Step 2: 7,10-Dioxadispiro[3.1.46.14]undecan-2-yl(diphenyl)methanol

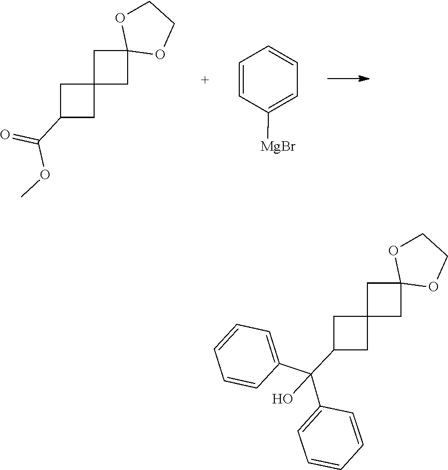

To a stirring solution of methyl 7,10-dioxadispiro[3.1.46.14]undecane-2-carboxylate (27.67 g, 117.33 mmol) in anhydrous diethyl ether (250 mL) at 0° C. under nitrogen was dropwise added a solution of bromo(phenyl)magnesium (135 mL of 3 M, 405.00 mmol) in diethyl ether. During this addition, the copious amount of precipitate was formed. After the addition was complete, the reaction mixture was stirred at this temperature for 10 minutes. The ice-water bath was removed, and the reaction mixture was heated to reflux (42° C.) for 2 hours. The reaction mixture was cooled to 0° C., and slowly quenched with saturated aqueous ammonium chloride (500 mL). The reaction mixture was allowed to warm up to room temperature and stirred until all the solid has dissolved. Two layers were separated, and the aqueous layer was extracted with diethyl ether (2×300 mL). The combined organic layers were washed with brine (150 mL), dried over anhydrous sodium sulfate and concentrated. The crude was purified by silica gel chromatography using 0-40% diethyl ether gradient in hexanes to afford 7,10-dioxadispiro[3.1.46.14]undecan-2-yl(diphenyl)methanol (28.07 g, 64%) as white solid. ESI-MS m/z calc. 336.1725, found 319.3 (M-water+H)+; Retention time: 5.77 minutes. ¹H NMR (250 MHz, CDCl₃) δ 7.47-7.10 (m, 1OH), 3.85 (s, 4H), 3.23 (p, J=8.7, 8.7, 8.6, 8.6 Hz, 1H), 2.40 (s, 2H), 2.25-2.10 (m, 5H), 2.04-1.89 (m, 2H). LC method S.

### Step 3: 2-Benzhydrylidene-7,10-dioxadispiro[3.1.46.14]undecane

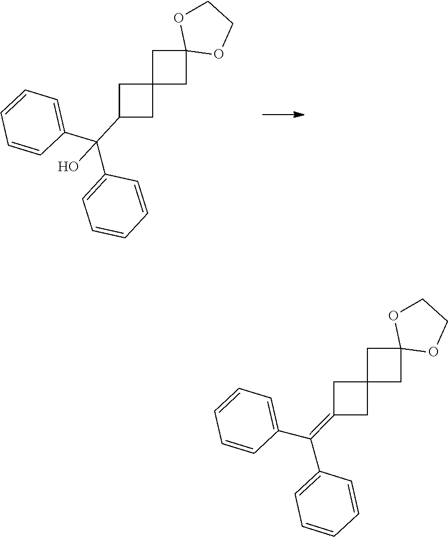 [SMILES: OC(c1ccccc1)(c1ccccc1)C1CC2(C1)CC1(C2)OCCO1 ; c1ccc(C(=C2CC3(C2)CC2(C3)OCCO2)c2ccccc2)cc1]

To a stirring solution of 7,10-dioxadispiro[3.1.46.14]undecan-2-yl(diphenyl)methanol (28.07 g, 83.436 mmol) in toluene (400 mL) at room temperature under ambient conditions was added p-toluenesulfonic acid hydrate (1.664 g, 8.7479 mmol). The reaction mixture was heated to reflux (140° C.) with Dean-Stark apparatus for 24 hours. After cooling to room temperature, volatiles were removed under vacuum. The obtained residue was dissolved in ethyl acetate (350 mL) and washed with saturated aqueous sodium bicarbonate (400 mL). Two layers were separated, and the aqueous layer was extracted with ethyl acetate (2×200 mL). The combined organic layers were washed with brine (150 mL), dried over anhydrous sodium sulfate and concentrated to afford 2-benzhydrylidene-7,10-dioxadispiro[3.1.46.14]undecane (26.645 g, 90%) as yellow solid. The product was carried to the next step without further purification. ESI-MS m/z calc. 318.162, found 319.0 (M+1)⁺; Retention time: 7.17 minutes ¹H NMR (250 MHz, CDCl₃) δ 7.43-7.04 (m, 10H), 3.88 (s, 4H), 3.03 (s, 4H), 2.43 (s, 4H). LC method S.

### Step 4: 7,10-Dioxadispiro[3.1.46.14]undecan-2-one

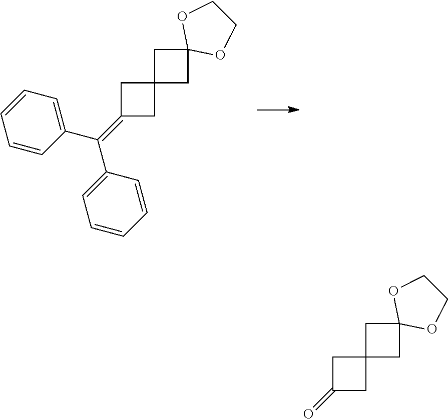 [SMILES: c1ccc(C(=C2CC3(C2)CC2(C3)OCCO2)c2ccccc2)cc1 ; O=C1CC2(C1)CC1(C2)OCCO1]

To a stirring solution of 2-benzhydrylidene-7,10-dioxadispiro[3.1.46.14]undecane (26.645 g, 83.682 mmol) in a mixture of acetonitrile (350 mL) and carbon tetrachloride (350 mL) at room temperature under ambient conditions was added water (550 mL). To the reaction mixture was added ruthenium(III) chloride hydrate (1.902 g, 8.4367 mmol), followed by a portionwise addition of sodium periodate (90.18 g, 421.61 mmol). After the addition was complete, the reaction mixture was stirred at this temperature for 5 minutes. The reaction mixture was heated to reflux (82° C.) for 1 hour. The reaction mixture was allowed to cool down to room temperature and filtered through a pad of Celite. The filter cake was washed with chloroform (3×200 mL). The combined filtrate was concentrated under vacuum to remove the volatiles. The residual aqueous layer was diluted with brine (200 mL), and the product was extracted with chloroform (3×400 mL). The combined organic layers were washed with brine (200 mL), dried over anhydrous sodium sulfate and concentrated. The crude was purified by silica gel chromatography using 0-20% acetone gradient in hexanes to afford 7,10-dioxadispiro[3.1.46.14]undecan-2-one (8.745 g, 59%) as a yellow oil. ¹H NMR (250 MHz, CDCl₃) δ 3.91 (s, 4H), 3.18 (s, 4H), 2.59 (s, 4H).

### Example Z: Preparation of tert-butyl N-[6-[[(1R)-1-(hydroxymethyl)-3,3-dimethyl-butyl]amino]spiro[3.3]heptan-2-yl]carbamate

### Step 1: tert-Butyl N-[6-[[(1R)-1-(hydroxymethyl)-3,3-dimethyl-butyl]amino]spiro[3.3]heptan-2-yl]carbamate

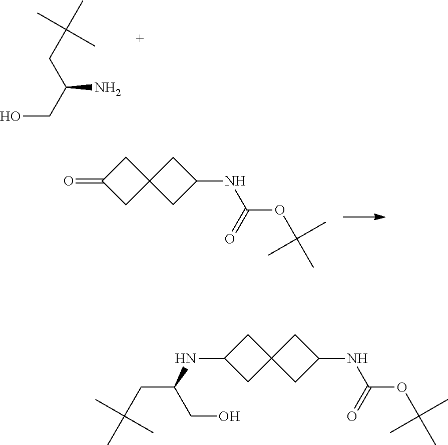

To a mixture of (2R)-2-amino-4,4-dimethyl-pentan-1-ol (hydrochloride salt) (55 g, 328.0 mmol) in 1,2-dichloroethane (600 mL) was added DIEA (60 mL, 344.5 mmol) and the mixture stirred for 5 min at ambient temperature. To the mixture was added tert-butyl N-(2-oxospiro[3.3]heptan-6-yl)carbamate (73.7 g, 327.1 mmol) followed by HOAc (20 mL, 351.7 mmol) and the homogenous mixture stirred for 2 hours. To the mixture was added sodium triacetoxyborohydride (83.6 g, 394.4 mmol) portionwise and the mixture stirred at ambient temperature for 2 hours. The mixture was cooled with an ice-water bath and quenched with water (600 mL) and stirred for 10 minutes. To the mixture was added HCl (60 mL of 12 M, 720.0 mmol) portionwise until mixture had a ˜pH 1. followed by isopropyl Acetate (600 mL). The mixture was basified with NaOH (160 g of 50% w/w, 2.000 mol) resulting in an emulsion. After adding NaCl, adjusting pH lower, and adding iPrOAc the organic phase was partially separated, and the solvent was removed in vacuo to about 250 mL. The aqueous phase was passed through a plug of Celite. The aqueous phase was extracted with 1 L of iPrOAc. The organic phases were combined and filtered through a plug of Celite. A small amount of water separated, and the organic phase was dried over magnesium sulfate, filtered over Celite and concentrated in vacuo affording light yellow molasses. It was diluted with MTBE (1,000 mL) and TsOH (42 g, 243.9 mmol) was added. The mixture was stirred at ambient temperature for 4 h. The off-white slurry was filtered using an M frit to give a solid paste. The precipitate was air dried for 20 h. The still damp solid was then diluted with MTBE (1000 mL) and the precipitate residue transferred with MeOH (100 mL). To the milky solution was added NaOH (350 mL of 2 M, 700.0 mmol) and the mixture stirred until no solid was observed. The organic phase was separated and the aqueous phase extracted with MTBE (1000 mL). The combined organic phases were washed with 300 mL of brine, dried over magnesium sulfate, filtered and concentrated in vacuo affording a light yellow oily foam of tert-butyl N-[6-[[(1R)-1-(hydroxymethyl)-3,3-dimethyl-butyl]amino]spiro[3.3]heptan-2-yl]carbamate (88.5 g, 79%). ¹H NMR (400 MHz, DMSO-d₆) δ 7.00 (d, J=8.0 Hz, 1H), 4.42 (s, 1H), 4.09 (q, J=5.4 Hz, OH), 3.78 (q, J=8.1 Hz, 1H), 3.26 (dd, J=10.7, 4.5 Hz, 1H), 3.21-3.10 (m, 2H), 2.42 (q, J=5.5, 5.0 Hz, 1H), 2.23 (dp, J=18.3, 6.7, 6.2 Hz, 2H), 2.05 (dt, J=11.6, 5.4 Hz, 2H), 1.82 (q, J=9.8, 9.3 Hz, 2H), 1.71-1.42 (m, 3H), 1.35 (s, 10H), 0.87 (s, 10H). ESI-MS m/z calc. 340.27258, found 341.3 (M+1)⁺; Retention time: 0.9 minutes (LC method A).

### Example AA: Preparation of tert-butyl N-[6-[[(1R)-1-(hydroxymethyl)-2-[1-(trifluoromethyl)cyclopropyl]ethyl]amino]spiro[3.3]heptan-2-yl]carbamate

### Step 1: tert-Butyl N-[6-[[(1R)-1-(hydroxymethyl)-2-[1-(trifluoromethyl)cyclopropyl]ethyl]amino]spiro[3.3]heptan-2-yl]carbamate

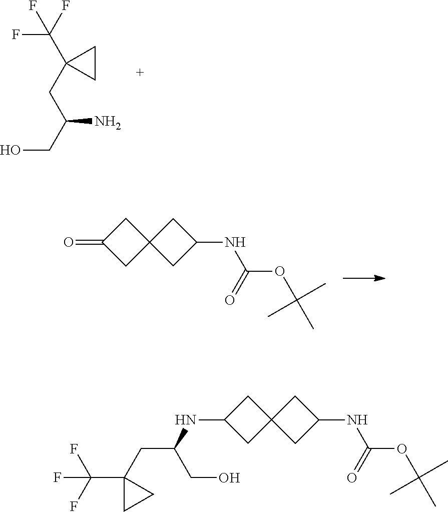 [SMILES: N[C@@H](CO)CC1(C(F)(F)F)CC1 ; CC(C)(C)OC(=O)NC1CC2(CC(=O)C2)C1 ; CC(C)(C)OC(=O)NC1CC2(C1)CC(N[C@@H](CO)CC1(C(F)(F)F)CC1)C2]

To a slurry of (2R)-2-amino-3-[1-(trifluoromethyl)cyclopropyl]propan-1-ol (hydrochloride salt) (96.2 g, 438.0 mmol) in 1,2-dichloroethane (1,000 mL) was added DIEA (80 mL, 459.3 mmol) and the mixture stirred for 5 minutes and became homogenous. To the mixture was added tert-butyl N-(2-oxospiro[3.3]heptan-6-yl)carbamate (98.6 g, 437.7 mmol) followed by HOAc (27 mL, 474.8 mmol) and the mixture stirred at ambient temperature for 1 hour. To the mixture was added sodium triacetoxyborohydride (106.8 g, 503.9 mmol) portions and the mixture stirred at ambient temperature (slow exotherm to 30° C. for 30 min, then cooled to ambient temperature). After 3 h, additional sodium triacetoxyborohydride (21.75 g, 102.6 mmol) was added and the reaction was stirred at ambient temperature for 14 hour. The mixture was cooled with an ice-water bath and quenched with water (1000 mL) and stirred for 10 min. To the mixture was added HCl (110 mL of 12 M, 1.320 mol) portions followed by isopropyl acetate (1,000 mL). The mixture was basified with NaOH (350 g of 50% w/w, 4.375 mol) and the phases split. The aqueous phase was extracted with isopropyl acetate (1,000 mL). The combined organic phases were washed with 1 L of brine, dried over magnesium sulfate, filtered and concentrated in vacuo. During concentration the product began to precipitate out and was collected using a M frit. The solid was washed twice with 50 mL of MTBE and the combined solids dried in vacuo at 45° C. The solid was diluted with MTBE (9 L) and TsOH (40 g, 232.3 mmol) was added. The creamy, white slurry was stirred for 30 minutes. The precipitate was collected using a M frit. The solid was air dried for 16 h. The solid was slurried with isopropyl acetate (700 mL) and NaOH (500 mL of 2 M, 1.000 mol) until homogenous. The phases were separated, and the organic phase washed with 500 mL of brine. The aqueous phases were extracted with isopropyl acetate (700 mL) and the combined organic phases were dried over magnesium sulfate, filtered and concentrated in vacuo to about 200 mL. The slurry was filtered and a second crop from the filtrate was also collected and were added to the first crop collected. tert-Butyl N-[6-[[(1R)-1-(hydroxymethyl)-2-[1-(trifluoromethyl)cyclopropyl]ethyl]amino]spiro[3.3]heptan-2-yl]carbamate (108.7 g, 63%). ¹H NMR (400 MHz, DMSO-d₆) δ 7.01 (d, J=8.0 Hz, 1H), 4.45 (q, J=5.0 Hz, 1H), 3.78 (h, J=8.3 Hz, 1H), 3.37-3.31 (m, 1H), 3.24 (dt, J=10.8, 5.3 Hz, 1H), 3.10 (p, J=7.5 Hz, 1H), 2.55 (q, J=5.7 Hz, 1H), 2.21 (dt, J=13.4, 6.0 Hz, 2H), 2.04 (p, J=5.6 Hz, 2H), 1.83 (q, J=9.8 Hz, 2H), 1.68-1.43 (m, 5H), 1.35 (s, 9H), 0.86 (s, 2H), 0.77 (d, J=11.1 Hz, 2H). ESI-MS m/z calc. 392.22867, found 393.2 (M+1)⁺; Retention time: 1.66 minutes (LC method A).

### Example BB: Preparation of 5-tetrahydropyran-4-ylpyridine-2-carbaldehyde

### Step 1: 5-(3,6-Dihydro-2H-pyran-4-yl)pyridine-2-carbaldehyde

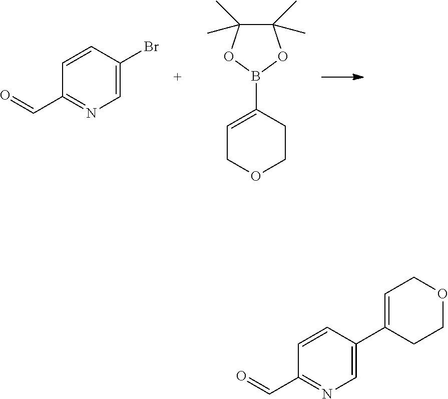

To a sealed tube was added 5-bromopyridine-2-carbaldehyde (2.5 g, 13.440 mmol), 2-(3,6-dihydro-2H-pyran-4-yl)-4,4,5,5-tetramethyl-1,3,2-dioxaborolane (3 g, 14.416 mmol) in degassed 1,4-dioxane (50 mL). A degassed solution of potassium carbonate (5.6 g, 40.519 mmol) in water (12.5 mL) was added followed by 1,1′-bis(diphenylphosphino)ferrocene palladium(II) chloride, complex with dichloromethane (220 mg, 0.2694 mmol). The tube was sealed and the reaction mixture was stirred at 80° C. for 18 hours and then cooled to room temperature. The mixture was filtered on Celite, the pad was rinsed with EtOAc (100 mL) and the filtrate was concentrated in vacuo. The mixture was triturated from MTBE (100 mL) and filtered. Afforded 5-(3,6-dihydro-2H-pyran-4-yl)pyridine-2-carbaldehyde (1.05 g, 39%) as a brown solid. ¹H NMR (400 MHz, CDCl₃) δ 10.08 (s, 1H), 8.84 (d, J=2.0 Hz, 1H), 7.96 (d, J=8.1 Hz, 1H), 7.84 (dd, J=8.1, 2.0 Hz, 1H), 6.44-6.33 (m, 1H), 4.39 (q, J=2.9 Hz, 2H), 3.99 (t, J=5.4 Hz, 2H), 2.71-2.49 (m, 2H). ESI-MS m/z calc. 189.079, found 190.2 (M+1)⁺; Retention time: 1.4 minutes. The filtrate was concentrated under reduced pressure and the resulting semi-solid was triturated from MTBE (15 mL) and filtered. Afforded 5-(3,6-dihydro-2H-pyran-4-yl)pyridine-2-carbaldehyde (0.6 g, 22%) as a brown solid. ¹H NMR (400 MHz, CDCl₃) δ 10.08 (s, 1H), 8.84 (d, J=2.0 Hz, 1H), 7.96 (d, J=8.1 Hz, 1H), 7.84 (dd, J=8.1, 2.0 Hz, 1H), 6.44-6.33 (m, 1H), 4.39 (q, J=2.9 Hz, 2H), 3.99 (t, J=5.4 Hz, 2H), 2.71-2.49 (m, 2H). LC method X.

### Step 2: (5-Tetrahydropyran-4-yl-2-pyridyl)methanol

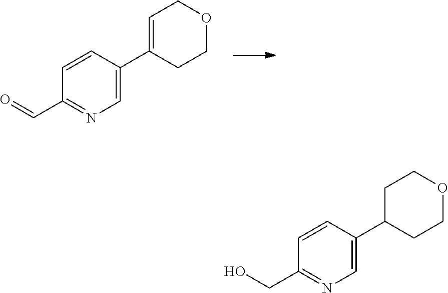 [SMILES: O=Cc1ccc(C2=CCOCC2)cn1 ; OCc1ccc(C2CCOCC2)cn1]

Palladium 5% on carbon (200 mg, 0.0940 mmol) was added into a 250 mL-flask and it was purged with nitrogen for 2 minutes. A solution of 5-(3,6-dihydro-2H-pyran-4-yl)pyridine-2-carbaldehyde (1.65 g, 8.5460 mmol) in methanol (30 mL) was then added into the flask. Hydrogen was bubbled into the suspension for 2 minutes and then the reaction mixture was stirred under hydrogen atmosphere for 18 hours. Nitrogen was then bubbled into the mixture for 10 minutes. The reaction mixture was filtered on Celite, the pad was rinsed with EtOAc (100 mL) and the filtrate was concentrated in vacuo. Afforded crude (5-tetrahydropyran-4-yl-2-pyridyl)methanol (1 g, 58%) as a light yellow solid. ¹H NMR (400 MHz, CDCl₃) δ 8.46 (d, J=1.7 Hz, 1H), 7.55 (dd, J=8.1, 2.2 Hz, 1H), 7.21 (d, J=8.1 Hz, 1H), 4.75 (d, J=3.2 Hz, 2H), 4.17-4.07 (m, 2H), 3.65-3.51 (m, 3H), 2.89-2.76 (m, 1H), 1.93-1.73 (m, 4H). ESI-MS m/z calc. 193.1103, found 194.2 (M+1)⁺; Retention time: 0.25 minutes (LC method X).

### Step 3: 5-Tetrahydropyran-4-ylpyridine-2-carbaldehyde

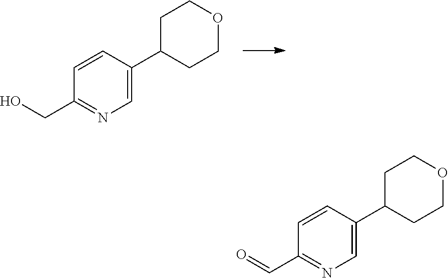 [SMILES: OCc1ccc(C2CCOCC2)cn1 ; O=Cc1ccc(C2CCOCC2)cn1]

To a solution of (5-tetrahydropyran-4-yl-2-pyridyl)methanol (1 g, 5.0714 mmol) in water saturated DCM (25 mL) was added Dess-Martin periodinane (2.39 g, 5.6349 mmol) and the reaction was then stirred for 1 hour at room temperature. A mixture of aqueous saturated solutions of Na₂S₂O₃ (20 mL), saturated aqueous sodium bicarbonate (20 mL), water (10 mL) and 1N aqueous NaOH (6 mL, to reach pH=7) was added and the reaction mixture was stirred for 10 minutes. The phases were separated, and the aqueous layer was extracted with DCM (3×50 mL). The combined organic layers were dried over sodium sulfate, filtered and concentrated in vacuo. The crude product was purified by reverse phase chromatography using a 100 g Cis cartridge, eluting with a gradient of MeCN in water (5% to 100%). Volatiles were removed from the fractions containing the product. The resulting aqueous phase was extracted with DCM (3×30 mL). The combined organic layers were washed with brine (30 mL), dried over sodium sulfate, filtered and concentrated in vacuo. Afforded 5-tetrahydropyran-4-ylpyridine-2-carbaldehyde (730 mg, 74%) as a white solid. ¹H NMR (400 MHz, CDCl₃) δ 10.08 (d, J=0.7 Hz, 1H), 8.68 (d, J=2.0 Hz, 1H), 7.95 (d, J=8.1 Hz, 1H), 7.73 (dd, J=8.1, 2.2 Hz, 1H), 4.19-4.08 (m, 2H), 3.57 (td, J=11.5, 2.7 Hz, 2H), 3.02-2.82 (m, 1H), 1.94-1.79 (m, 4H). ESI-MS m/z calc. 191.0946, found 192.1 (M+1)⁺; Retention time: 2.24 minutes (LC method X).

### Example CC: Preparation of (2R)-2-[(3-Benzyloxycyclobutyl)amino]-4-methyl-pentan-1-ol

### Step 1: (2R)-2-[(3-Benzyloxycyclobutyl)amino]-4-methyl-pentan-1-ol

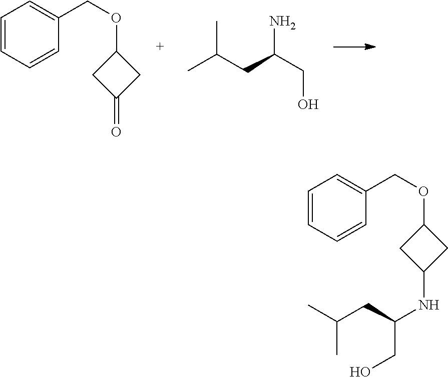

Into a solution of (2R)-2-amino-4-methyl-pentan-1-ol (2.0 g, 17.066 mmol) in anhydrous DCE (25 mL) was added 3-benzyloxycyclobutanone (2.389 g, 13.558 mmol). The reaction was stirred at room temperature for 30 minutes. Sodium triacetoxyborohydride (6.32 g, 29.820 mmol) was added to the reaction, and then it was stirred at room temperature overnight. The reaction was poured into 2 N sodium carbonate (30 mL). The reaction was extracted with DCM (3×30 mL). The combined organic layers were dried over anhydrous sodium sulfate and concentrated under vacuum. The residue was purified by silica gel chromatography using 0 to 10% methanol in DCM (buffered with 0.2% ammonium hydroxide) to furnish (2R)-2-[(3-benzyloxycyclobutyl)amino]-4-methyl-pentan-1-ol (3.379 g, 69%) as a clear oil. ESI-MS m/z calc. 277.2042, found 278.3 (M+1)⁺; Retention time: 3.68 minutes; LC method S.

### Example DD: Preparation of tert-butyl 2-[[(1R)-1-(hydroxymethyl)-3-methyl-butyl]amino]-7-azaspiro[3.5]nonane-7-carboxylate

### Step 1: tert-Butyl 2-[[(1R)-1-(hydroxymethyl)-3-methyl-butyl]amino]-7-azaspiro[3.5]nonane-7-carboxylate

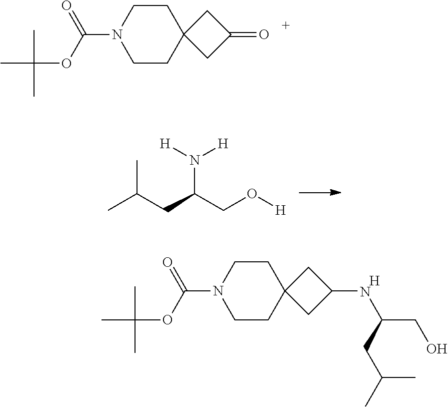

(2R)-2-Amino-4-methyl-pentan-1-ol (4.0 mL, 31.30 mmol) was added to a solution of tert-butyl 2-oxo-7-azaspiro[3.5]nonane-7-carboxylate (5.00 g, 20.89 mmol) in anhydrous DCE (30 mL) under nitrogen and stirred at rt for 30 minutes. Sodium triacetoxyborohydride (6.64 g, 31.33 mmol) was added and the reaction was stirred at rt for 1 hour 45 minutes, then another portion of sodium triacetoxyborohydride (3.33 g, 15.71 mmol) was added and stirred for 2 hours. A third portion of sodium triacetoxyborohydride (3.33 g, 15.71 mmol) was added and it was stirred for 2 hours. HCl (84 mL of 1 M, 84.00 mmol) was added and stirred for 10 minutes, then a solution of potassium carbonate (12.13 g, 87.77 mmol) in water (20 mL) was added. The organic layer was separated, and the aqueous layer was extracted with DCM (30 mL). The organic layers were combined and dried over magnesium sulfate, then concentrated to give tert-butyl 2-[[(1R)-1-(hydroxymethyl)-3-methyl-butyl]amino]-7-azaspiro[3.5]nonane-7-carboxylate (8.79 g, 108%) ESI-MS m/z calc. 340.27258, found 341.3 (M+1)⁺; Retention time: 1.19 minutes; LC method A.

### Example EE: Preparation of 5-morpholinopyridine-2-carbaldehyde

### Step 7: 5-Morpholinopyridine-2-carbaldehyde

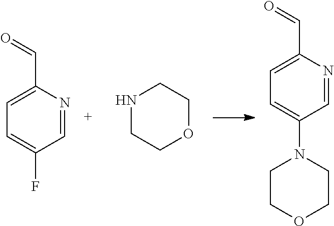 [SMILES: O=Cc1ccc(F)cn1 ; C1COCCN1 ; O=Cc1ccc(N2CCOCC2)cn1]

5-Fluoropyridine-2-carbaldehyde (5 g, 39.97 mmol) was combined with potassium carbonate (22.1 g, 159.9 mmol) and morpholine (7 mL, 80.27 mmol) in DMF (50 mL), and the reaction mixture was heated to 110° C. for hours. After cooling to room temperature, the reaction was diluted with methanol, filtered, and purified. A small quantity of water was added to the filtrate, which was then concentrated under reduced pressure. The resulting crude material was purified by chromatography on silica gel, eluting with a 0-10% gradient of methanol in DCM, to give 5-morpholinopyridine-2-carbaldehyde (6.389 g, 83%) as a light brown solid. ESI-MS m/z calc. 192.08987, found 193.2 (M+1)⁺; Retention time: 0.27 minutes, LC method D.

### Example FF: Preparation of 5-morpholinopyrimidine-2-carbaldehyde

### Step 1: 5-Fluoropyrimidine-2-carbaldehyde

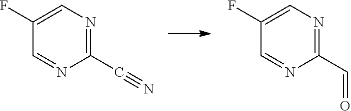 [SMILES: N#Cc1ncc(F)cn1 ; O=Cc1ncc(F)cn1]

Into a solution of 5-fluoropyrimidine-2-carbonitrile (10 g, 77.993 mmol) in anhydrous THE (200 mL) was added 1.0 M DIBAL-H in toluene (117 mL of 1 M, 117.00 mmol) at −78° C. dropwise for 30 minutes. After the addition, the reaction was stirred for another 2 hours at the same temperature. Methanol (40 mL) was added to the reaction mixture at −78° C. The reaction temperature was slowly raised to rt, and then it was diluted with 10% HCl (aqueous) (60 mL) and concentrated HCl (20 mL) (pH=3). Solid NaCl was added to saturate the aqueous layer. The reaction mixture was stirred for 1 hours until both layers were clear. Two layers were separated. The aqueous layer was extracted with DCM (10×100 mL). The combined organic layers were dried over anhydrous sodium sulfate and concentrated under vacuum. The residue toluene solution was directly loaded onto a silica gel column and purified using 0 to 60% diethyl ether in DCM. The correct fractions were combined and concentrated under vacuum to furnish 5-fluoropyrimidine-2-carbaldehyde (5.545 g, 54%) as a yellow liquid. ESI-MS m/z calc. 126.0229, found 127.2 (M+1)⁺; Retention time: 0.34 minutes. ¹H NMR (500 MHz, DMSO-d₆) δ 9.96 (s, 1H), 9.17 (d, J=0.8 Hz, 2H). LC method W.

### Step 2: 5-Morpholinopyrimidine-2-carbaldehyde

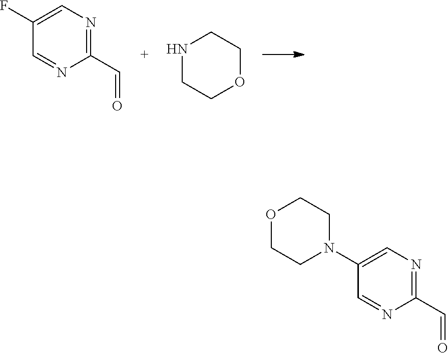 [SMILES: O=Cc1ncc(F)cn1 ; C1COCCN1 ; O=Cc1ncc(N2CCOCC2)cn1]

Into a solution of 5-fluoropyrimidine-2-carbaldehyde (1.29 g, 6.6194 mmol) in anhydrous DMF (10 mL) was added morpholine (1.1988 g, 1.2 mL, 13.760 mmol) and potassium carbonate (3.65 g, 26.410 mmol). The reaction was stirred at 110° C. for 4 hours. After centrifuge, the DMF solution was directly subjected to HPLC purification using 0 to 40% ACN in water (buffered with 0.1% HCl). The correct fractions were combined and lyophilized to furnish 5-morpholinopyrimidine-2-carbaldehyde (hydrochloride salt) (1.4515 g, 91%) as a yellow solid. ESI-MS m/z calc. 193.0851, found 194.3 (M+1)⁺; Retention time: 1.27 minutes. ¹H NMR (500 MHz, DMSO-d₆) δ 9.79 (s, 1H), 8.65 (s, 2H), 3.82-3.66 (m, 4H), 3.54-3.37 (m, 4H). LC method W.

### Example GG: Preparation of (1R,2R)-2-Amino-1-(4-tert-butylphenyl)propan-1-ol (hydrochloride salt)

### Step 1: tert-Butyl N-[(1R)-1-methyl-2-oxo-ethyl]carbamate

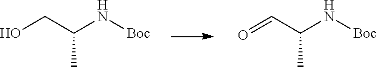

To a solution of tert-butyl N-[(1R)-2-hydroxy-1-methyl-ethyl]carbamate (200 g, 1.141 mol) in DCM (3 L) was added Dess-Martin periodinane (625 g, 1.474 mol) (fine suspension, most into solution, started exotherm, controlled with ice-bath). To the mixture was added water (28 mL, 1.554 mol) slowly added over 0.5 hours (exothermic during addition up to 33° C., kept between 20 and 33° C. by cooling with cold water) giving a colorless thick suspension. The suspension was stirred at room temperature for 16 hours. The solid was removed by filtration over Celite and washed 3× with 100 mL of DCM. The solvent was removed in vacuo affording an off-white slurry, which was diluted with MTBE (750 mL). The slurry was cooled with an ice-bath and filtered over Celite. The filtrate was washed 3× with sat sodium bicarbonate, brine, dried over magnesium sulfate, filtered and concentrated in vacuo. The semi-solid was re-dissolved in MTBE (300 mL) and diluted with heptane (750 mL). The solution was concentrated in vacuo until a cloud point occurred. The slurry was stirred at ambient temperature for 0.5 hours. The precipitate was collected, washed with cold heptane and dried in vacuo at ambient temperature (this solid was product and was therefore kept aside). The filtrate was further concentrated in vacuo until a cloud point occurred. The solution was allowed to stand for 48 h affording a thick off-white slurry. The slurry was filtered, and the filter cake was washed with ˜50 mL of cold heptane. The filter cake was combined with the solid kept aside earlier and air-dried for 4 hours. Product contained approximately 9% residual heptane by ¹H NMR. tert-Butyl N-[(1R)-1-methyl-2-oxo-ethyl]carbamate (95.6 g, 48%), ¹H NMR (500 MHz, DMSO-d₆) δ 9.43 (s, 1H), 7.35 (d, J=6.8 Hz, 1H), 3.86 (t, J=7.2 Hz, 1H), 1.40 (s, 9H), 1.13 (d, J=7.3 Hz, 3H).

### Step 2: tert-Butyl N-[(1R,2R)-2-(4-tert-butylphenyl)-2-hydroxy-1-methyl-ethyl]carbamate

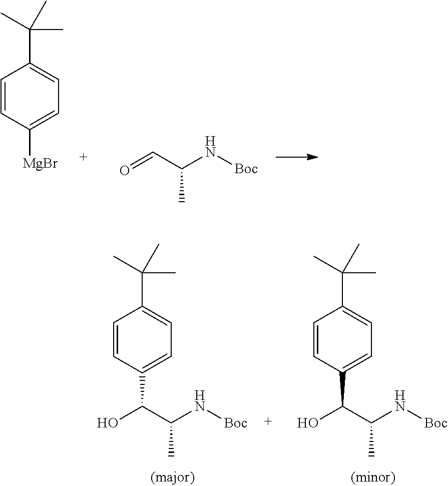

A solution of tert-butyl N-[(1R)-1-methyl-2-oxo-ethyl]carbamate (101.73 g, 587.3 mmol) in MeTHF (500 mL) was added slowly over 1 h to bromo-(4-tert-butylphenyl)magnesium (1300 mL of 1 M, 1.300 mol) (1 M in MeTHF) in a −35° C. cold bath at a rate which maintained an internal temperature between −2° C. and −15° C. After the addition was complete, it was stirred for 5 min, then the mixture was removed from the cold bath and transferred to a room temperature water bath, then stirred for 2.5 hours. The mixture was cooled to 0° C., then saturated ammonium chloride (1700 mL) was added (large exotherm) at a rate which maintained an internal temperature of 5° C. Water (500 mL) was added, the organic layer was separated and washed with brine (500 mL), dried over magnesium sulfate, then concentrated under vacuum to give a light yellow oil, tert-butyl N-[(1R,2R)-2-(4-tert-butylphenyl)-2-hydroxy-1-methyl-ethyl]carbamate (266 g, >100% yield), which was used in the next step without further purification. ESI-MS m/z calc. 307.21475, found 308.1 (M+1)⁺; Retention time: 1.86 minutes; LC method A.

### Step 3: (1R,2R)-2-Amino-1-(4-tert-butylphenyl)propan-1-ol (hydrochloride salt)

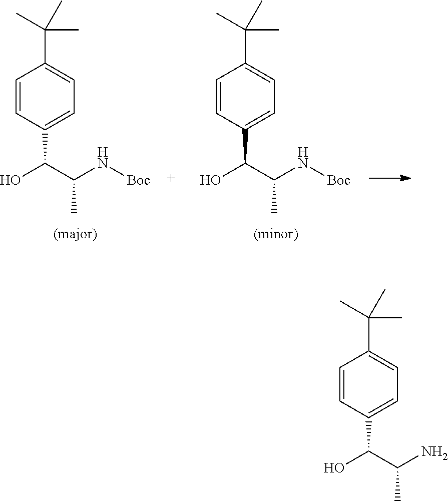 [SMILES: C[C@@H](NC(=O)OC(C)(C)C)[C@H](O)c1ccc(C(C)(C)C)cc1 ; C[C@@H](NC(=O)OC(C)(C)C)[C@@H](O)c1ccc(C(C)(C)C)cc1 ; C[C@@H](N)[C@H](O)c1ccc(C(C)(C)C)cc1]

A solution of tert-butyl N-[(1R,2R)-2-(4-tert-butylphenyl)-2-hydroxy-1-methyl-ethyl]carbamate (180.6 g, 587.5 mmol) in MeOH (250 mL) was added dropwise over 50 min to HCl in dioxane (478 mL of 4 M, 1.912 mol), maintaining a temperature between 18° C. and 23° C., then stirred at room temperature for 2 hours. The mixture was concentrated under vacuum to give 267.5 g of residue. This was recrystallized from dioxane, the product was collected by filtration, then rinsed with MeTHF until all the color was removed, giving 75.4 g of product. This was further recrystallized from MeOH/dioxane, which gave (1R,2R)-2-amino-1-(4-tert-butylphenyl)propan-1-ol (hydrochloride salt) (62.65 g, 44%); ¹H NMR (500 MHz, DMSO-d₆) δ 8.10 (s, 3H), 7.39 (d, J=8.2 Hz, 2H), 7.28 (d, J=8.1 Hz, 2H), 6.12 (d, J=3.8 Hz, 1H), 4.50-4.34 (m, 1H), 3.28-3.12 (m, 1H), 1.27 (s, 9H), 0.96 (d, J=6.6 Hz, 3H). ESI-MS m/z calc. 207.16231, found 208.2 (M+1)⁺; Retention time: 1.01 minutes; LC method A.

### V. Synthesis of New Compounds

### Example 1: Preparation of Compound 1

### Step 1: N-(4,6-Dichloro-5-ethyl-pyrimidin-2-yl)-3-nitro-benzenesulfonamide

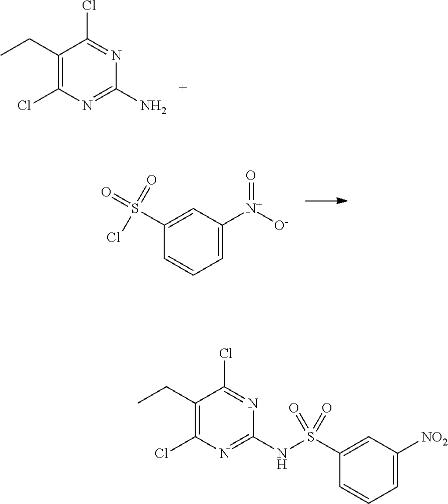 [SMILES: CCc1c(Cl)nc(N)nc1Cl ; O=[N+]([O-])c1cccc(S(=O)(=O)Cl)c1 ; CCc1c(Cl)nc(NS(=O)(=O)c2cccc([N+](=O)[O-])c2)nc1Cl]

To a solution of 4,6-dichloro-5-ethyl-pyrimidin-2-amine (5.05 g, 26.30 mmol) in DMF (105 mL) at 0° C. was added in one portion sodium hydride (4.2 g of 60% w/w, 105.0 mmol). The reaction was allowed to warm to 23° C. over 15 minutes. The reaction mixture was cooled to 0° C. again before adding 3-nitrobenzenesulfonyl chloride (11.7 g, 52.79 mmol). Stirring continued for 15 minutes and the reaction was quenched with acetic acid (15.8 g, 263.1 mmol) and water (100 mL). The crude solution was extracted with ethyl acetate (5×). The combined organics were washed with brine (3×), dried over magnesium sulfate, filtered, and concentrated in vacuo. The crude residue was separated by flash column chromatography on silica gel (10% methanol in dichloromethane) which afforded N-(4,6-dichloro-5-ethyl-pyrimidin-2-yl)-3-nitro-benzenesulfonamide (8.76 g, 79%) as a pale-yellow solid. ESI-MS m/z calc. 375.97998, found 377.05 (M+1)⁺; Retention time: 0.69 minutes; LC method D.

### Step 2: Methyl 3-[2-[(3-aminophenyl)sulfonylamino]-6-chloro-5-ethyl-pyrimidin-4-yl]oxybenzoate

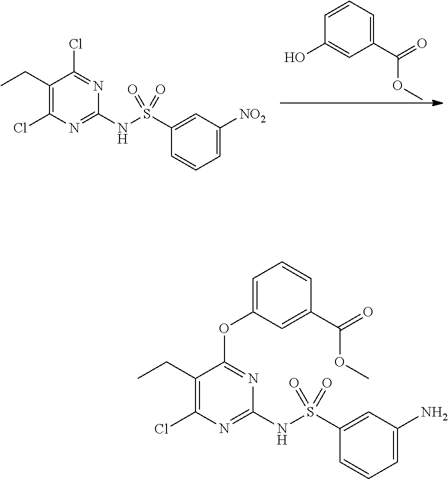 [SMILES: CCc1c(Cl)nc(NS(=O)(=O)c2cccc([N+](=O)[O-])c2)nc1Cl ; COC(=O)c1cccc(O)c1 ; CCc1c(Cl)nc(NS(=O)(=O)c2cccc(N)c2)nc1Oc1cccc(C(=O)OC)c1]

Stage 1: A heterogeneous solution of N-(4,6-dichloro-5-ethyl-pyrimidin-2-yl)-3-nitro-benzenesulfonamide (8.76 g, 20.67 mmol), methyl 3-hydroxybenzoate (3.2 g, 21.03 mmol), and potassium carbonate (8.6 g, 62.23 mmol) in NMP (21 mL) was heated to 120° C. for 16 h. The reaction mixture was diluted with dichloromethane (100 mL) and water (100 mL) and acidified with acetic acid (7.5 g, 124.9 mmol). The organic layer was separated, and the aqueous layer was further extracted with dichloromethane (4×). The combined organics were washed twice with brine, dried using magnesium sulfate, filtered, and concentrated in vacuo. The crude residue containing methyl 3-[6-chloro-5-ethyl-2-[(3-nitrophenyl)sulfonylamino]pyrimidin-4-yl]oxybenzoate was used without further purification. ESI-MS m/z calc. 492.05066, found 493.16 (M+1)⁺; Retention time: 0.77 minutes; LC method D.

Stage 2: To the crude residue from Stage 1 in methanol (82 mL) was added iron (5.8 g, 103.9 mmol) and hydrochloric acid (5.1 mL of 37% w/v, 51.75 mmol). The reaction was stirred at 23° C. for 16 hours. The reaction mixture was diluted with diethyl ether (100 mL), filtered, and concentrated in vacuo onto silica gel. The crude impregnated silica was submitted to flash column chromatography (gradient: 10 to 50% ethyl acetate in hexanes) which afforded methyl 3-[2-[(3-aminophenyl)sulfonylamino]-6-chloro-5-ethyl-pyrimidin-4-yl]oxybenzoate (5.19 g, 54%) as a yellow gel. ESI-MS m/z calc. 462.07648, found 463.2 (M+1)⁺; Retention time: 0.68 minutes; LC method D.

### Step 3: 5-Chloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione

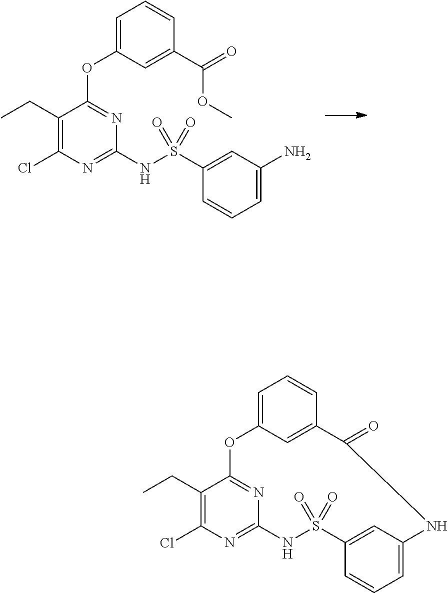 [SMILES: CCc1c(Cl)nc(NS(=O)(=O)c2cccc(N)c2)nc1Oc1cccc(C(=O)OC)c1 ; CCc1c(Cl)nc2nc1Oc1cccc(c1)C(=O)Nc1cccc(c1)S(=O)(=O)N2]

Stage 1: To a solution of methyl 3-[2-[(3-aminophenyl)sulfonylamino]-6-chloro-5-ethyl-pyrimidin-4-yl]oxybenzoate (5.19 g, 11.21 mmol) in THF (22.4 mL) and water (22.4 mL) was added sodium hydroxide (4.48 mL of 5 M, 22.40 mmol). The solution was heated to 65° C. for 3 hours. After cooling to 23° C., the solution was acidified with hydrochloric acid (3.74 mL of 6 M, 22.44 mmol) and concentrated in vacuo. The residual moisture was removed through azeotropic distillation with ethanol in vacuo which provided 3-[2-[(3-aminophenyl)sulfonylamino]-6-chloro-5-ethyl-pyrimidin-4-yl]oxybenzoic acid as a white solid (purity: 95% by UV-VIS) ESI-MS m/z calc. 448.06082, found 449.15 (M+1)⁺; Retention time: 0.6 minutes; LC method D.

Stage 2: To the crude intermediate from Stage 1 dissolved in DMF (210 mL) was added HATU (8.53 g, 22.43 mmol) followed by DIPEA (5.788 g, 44.78 mmol). The reaction solution was stirred for 20 minutes before diluting with ethyl acetate (1 L), brine (100 mL), and water (100 mL). The water layer was separated and the organic layer was further washed with brine (2×), dried over magnesium sulfate, filtered, and concentrated in vacuo. The crude residue was purified by flash column chromatography on silica gel (gradient: 10 to 100% ethyl acetate in hexanes) which afforded 5-chloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione (900 mg, 19%) as a white solid. ESI-MS m/z calc. 430.05026, found 431.11 (M+1)⁺; Retention time: 0.59 minutes; LC method D.

### Step 4: 5-(2-Cyclobutylphenyl)-4-ethyl-9,9-dioxo-2-oxa-9λ⁶-thia-6,8,15,23-tetrazatetracyclo[15.3.1.13,7.110,14]tricosa-1(21),3,5,7(23),10,12,14(22),17,19-nonaen-16-one (Compound 1)

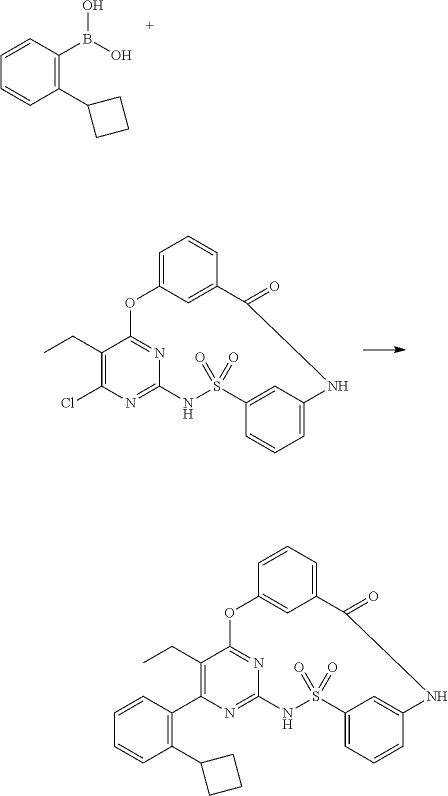 [SMILES: OB(O)c1ccccc1C1CCC1 ; CCc1c(Cl)nc2nc1Oc1cccc(c1)C(=O)Nc1cccc(c1)S(=O)(=O)N2 ; CCc1c2nc(nc1-c1ccccc1C1CCC1)NS(=O)(=O)c1cccc(c1)NC(=O)c1cccc(c1)O2]

A heterogeneous solution of (2-cyclobutylphenyl)boronic acid (approximately 30.65 mg, 0.1741 mmol), 5-chloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trion (25 mg, 0.05802 mmol), potassium carbonate (approximately 40.09 mg, 0.2901 mmol), and tetrakis(triphenylphosphine)palladium(0) (approximately 13.40 mg, 0.01160 mmol) in dioxane (194.5 μL) and water (39.00 μL) was heated in a sealed vial in a microwave to 120° C. for 30 minutes. DMSO (0.50 mL) was added and the crude solution was filtered and then separated by HPLC (acetonitrile in water with 0.1% hydrochloric acid) to give 5-(2-cyclobutylphenyl)-4-ethyl-9,9-dioxo-2-oxa-9λ⁶-thia-6,8,15,23-tetrazatetracyclo[15.3.1.13,7.110,14]tricosa-1(21),3,5,7(23),10,12,14(22),17,19-nonaen-16-one (8.3 mg, 27%); ESI-MS m/z calc. 526.1675, found 527.5 (M+1)⁺; Retention time: 1.79 minutes; LC method A.

### Example 2: Preparation of Compound 2

### Step 1: 4-Ethyl-9,9-dioxo-5-(2,3,4,5,6-pentamethylphenyl)-2-oxa-9λ⁶-thia-6,8,15,23-tetrazatetracyclo[15.3.1.13,7.110,14]tricosa-1(21),3,5,7(23),10,12,14(22),17,19-nonaen-16-one (Compound 2)

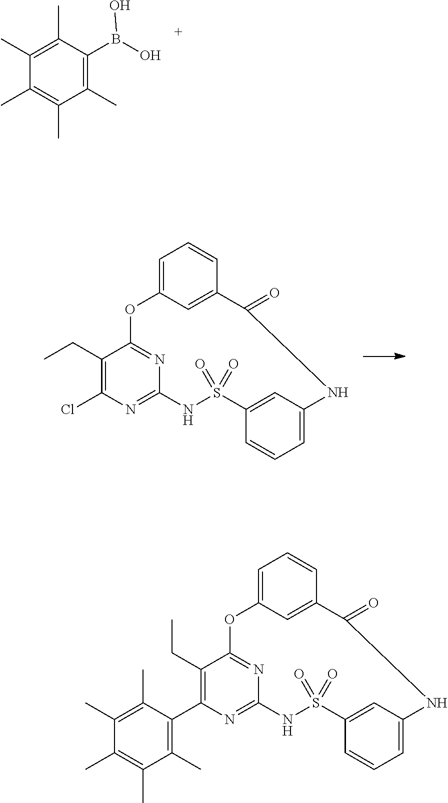 [SMILES: Cc1c(C)c(C)c(B(O)O)c(C)c1C ; CCc1c(Cl)nc2nc1Oc1cccc(c1)C(=O)Nc1cccc(c1)S(=O)(=O)N2 ; CCc1c2nc(nc1-c1c(C)c(C)c(C)c(C)c1C)NS(=O)(=O)c1cccc(c1)NC(=O)c1cccc(c1)O2]

A heterogeneous solution of (2,3,4,5,6-pentamethylphenyl)boronic acid (approximately 33.44 mg, 0.1741 mmol), 5-chloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione (25 mg, 0.05802 mmol), potassium carbonate (approximately 40.09 mg, 0.2901 mmol), and tetrakis(triphenylphosphine)palladium(0) (approximately 13.40 mg, 0.01160 mmol) in dioxane (194.5 μL) and water (39.00 μL) was heated in a sealed vial in a microwave to 120° C. for 30 minutes. DMSO (0.50 mL) was added and the crude solution was filtered and then separated by HPLC (acetonitrile in water with 0.1% hydrochloric acid) to give 4-ethyl-9,9-dioxo-5-(2,3,4,5,6-pentamethylphenyl)-2-oxa-9λ⁶-thia-6,8,15,23-tetrazatetracyclo[15.3.1.13,7.110,14]tricosa-1(21),3,5,7(23),10,12,14(22),17,19-nonaen-16-one (4.2 mg, 13%); ESI-MS m/z calc. 542.1988, found 543.53 (M+1)⁺; Retention time: 1.86 minutes; LC method A.

### Example 3: Preparation of Compound 3

### Step 1: 4-Ethyl-5-(2-isobutylphenyl)-9,9-dioxo-2-oxa-9λ⁶-thia-6,8,15,23-tetrazatetracyclo[15.3.1.13,7.110,14]tricosa-1(21),3,5,7(23),10,12,14(22),17,19-nonaen-16-one (Compound 3)

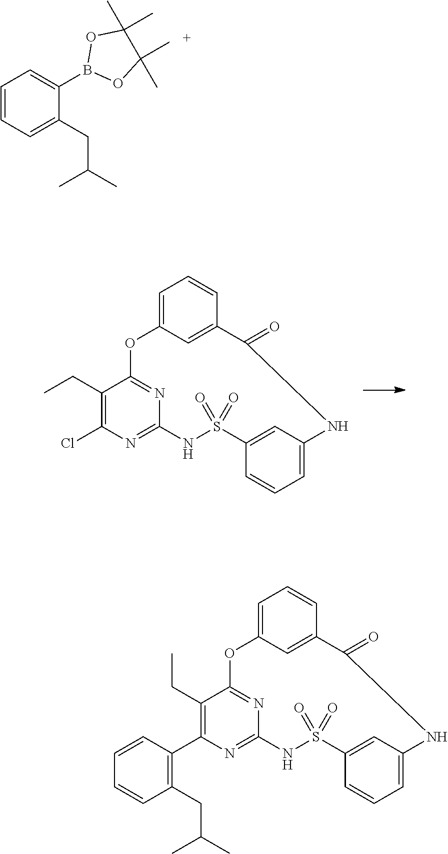 [SMILES: CC(C)Cc1ccccc1B1OC(C)(C)C(C)(C)O1 ; CCc1c(Cl)nc2nc1Oc1cccc(c1)C(=O)Nc1cccc(c1)S(=O)(=O)N2 ; CCc1c2nc(nc1-c1ccccc1CC(C)C)NS(=O)(=O)c1cccc(c1)NC(=O)c1cccc(c1)O2]

A heterogeneous solution of 2-(2-isobutylphenyl)-4,4,5,5-tetramethyl-1,3,2-dioxaborolane (approximately 45.30 mg, 0.1741 mmol), 5-chloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione (25 mg, 0.05802 mmol), potassium carbonate (approximately 40.09 mg, 0.2901 mmol), and tetrakis(triphenylphosphine)palladium(0) (approximately 13.40 mg, 0.01160 mmol) in dioxane (194.5 μL) and water (39.00 μL) was heated in a sealed vial in a microwave to 120° C. for 30 min. DMSO (0.50 mL) was added and the crude solution was filtered and then separated by HPLC (acetonitrile in water with 0.1% hydrochloric acid) to give 4-ethyl-5-(2-isobutylphenyl)-9,9-dioxo-2-oxa-9λ⁶-thia-6,8,15,23-tetrazatetracyclo[15.3.1.13,7.110,14]tricosa-1(21),3,5,7(23),10,12,14(22),17,19-nonaen-16-one (16.8 mg, 51%); ESI-MS m/z calc. 528.1831, found 529.53 (M+1)⁺; Retention time: 1.85 minutes; LC method A.

### Example 4: Preparation of Compound 4

### Step 1: 5-(2,6-Dimethylphenyl)-4-ethyl-9,9-dioxo-2-oxa-9λ⁶-thia-6,8,15,23-tetrazatetracyclo[15.3.1.13,7.110,14]tricosa-1(21),3,5,7(23),10,12,14(22),17,19-nonaen-16-one (Compound 4)

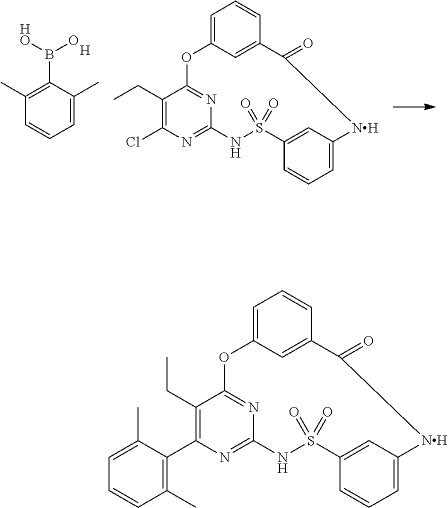

A heterogeneous solution of (2,6-dimethylphenyl)boronic acid (approximately 26.11 mg, 0.1741 mmol), 5-chloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione (25 mg, 0.05802 mmol), potassium carbonate (approximately 40.09 mg, 0.2901 mmol), and tetrakis(triphenylphosphine)palladium(0) (approximately 13.40 mg, 0.01160 mmol) in dioxane (194.5 μL) and water (39.00 μL) was heated in a sealed vial in a microwave to 120° C. for 30 minutes. DMSO (0.50 mL) was added and the crude solution was filtered and then separated by HPLC (acetonitrile in water with 0.1% hydrochloric acid) to give 5-(2,6-dimethylphenyl)-4-ethyl-9,9-dioxo-2-oxa-9λ⁶-thia-6,8,15,23-tetrazatetracyclo[15.3.1.13,7.110,14]tricosa-1(21),3,5,7(23),10,12,14(22),17,19-nonaen-16-one (4.9 mg, 16%); ESI-MS m/z calc. 500.15182, found 501.48 (M+1)⁺; Retention time: 1.6 minutes; LC method A.

### Example 5: Preparation of Compound 5

### Step 1: 5,18-Dichloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione

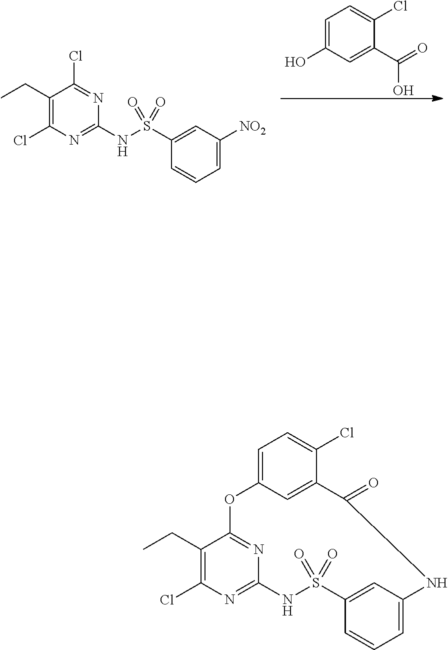 [SMILES: CCc1c(Cl)nc(NS(=O)(=O)c2cccc([N+](=O)[O-])c2)nc1Cl ; O=C(O)c1cc(O)ccc1Cl ; CCc1c(Cl)nc2nc1Oc1ccc(Cl)c(c1)C(=O)Nc1cccc(c1)S(=O)(=O)N2]

Stage 1: A heterogeneous mixture consisting of N-(4,6-dichloro-5-ethyl-pyrimidin-2-yl)-3-nitro-benzenesulfonamide (500 mg, 1.326 mmol), 2-chloro-5-hydroxy-benzoic acid (242 mg, 1.402 mmol), and potassium carbonate (919 mg, 6.650 mmol) in NMP (16 mL) was heated in a sealed vial to 150° C. for 16 h. After the reaction was cooled, it was diluted with water and ethyl acetate/hexanes (1:1). The mixture was acidified with hydrochloric acid (1.05 mL of 37% w/v, 10.7 mmol). The organic layer was separated and the aqueous layer further extracted with ethyl acetate/hexanes (1:1, 4×). The combined organics were washed with brine, dried over magnesium sulfate, filtered, and concentrated in vacuo. This afforded the intermediate 2-chloro-5-[6-chloro-5-ethyl-2-[(3-nitrophenyl)sulfonylamino]pyrimidin-4-yl]oxy-benzoic acid which was used without further purification.

Stage 2: To 2-chloro-5-[6-chloro-5-ethyl-2-[(3-nitrophenyl)sulfonylamino]pyrimidin-4-yl]oxy-benzoic acid from Stage 1 in ethanol (16 mL) was added iron (371 mg, 6.64 mmol) followed by hydrochloric acid (330 μL of 37% w/v, 3.35 mmol). The reaction was stirred for 16 hours. The reaction mixture was diluted with ether, filtered through Celite, and concentrated in vacuo. The intermediate 5-[2-[(3-aminophenyl)sulfonylamino]-6-chloro-5-ethyl-pyrimidin-4-yl]oxy-2-chloro-benzoic acid was used without further purification.

Stage 3: To the intermediate 5-[2-[(3-aminophenyl)sulfonylamino]-6-chloro-5-ethyl-pyrimidin-4-yl]oxy-2-chloro-benzoic acid from Stage 2 in DMF (8 mL) was added HATU (1.0 g, 2.6 mmol) followed by DIPEA (700 μL, 4.02 mmol). The reaction was stirred for 25 min before diluting with water and ethyl acetate/hexanes (1:1). The organic layer was separated and the aqueous layer was further extracted with ethyl acetate/hexanes (1:1, 5×). The combined organic extracts were washed twice with brine, dried over magnesium sulfate, filtered and concentrate in vacuo. The crude residue was separated by flash column chromatography on silica gel (40% ethyl acetate in hexanes) which afforded 5,18-dichloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione (100.3 mg, 7%) as a yellow solid. ESI-MS m/z calc. 464.0113, found 465.35 (M+1)⁺; Retention time: 0.6 minutes; LC method D.

### Step 2: 18-Chloro-4-ethyl-5-[2-(propan-2-yl)phenyl]-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione (Compound 5)

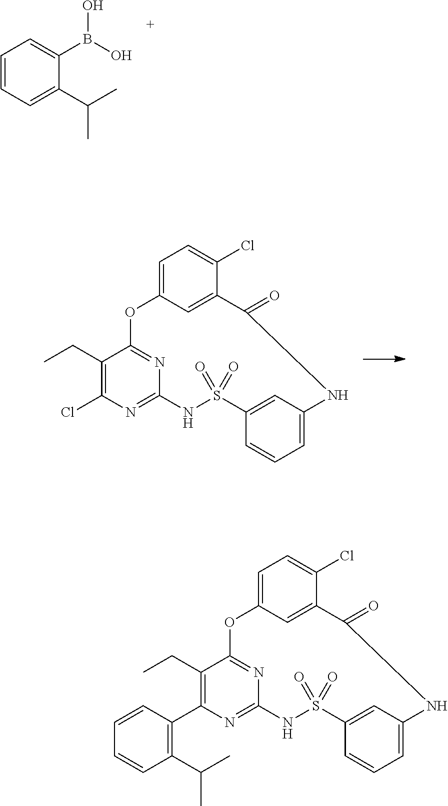 [SMILES: CC(C)c1ccccc1B(O)O ; CCc1c(Cl)nc2nc1Oc1ccc(Cl)c(c1)C(=O)Nc1cccc(c1)S(=O)(=O)N2 ; CCc1c2nc(nc1-c1ccccc1C(C)C)NS(=O)(=O)c1cccc(c1)NC(=O)c1cc(ccc1Cl)O2]

A heterogeneous solution of 5,18-dichloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione (25 mg, 0.035 mmol), (2-isopropylphenyl)boronic acid (6.9 mg, 0.04207 mmol), tetrakis(triphenylphosphine)palladium(0) (8.1 mg, 0.00701 mmol), and potassium carbonate (19 mg, 0.1375 mmol) in dioxane (160 μL) and water (30 μL) was microwaved in a sealed vial to 120° C. for 30 min. The reaction mixture was acidified with acetic acid (31 mg, 0.5162 mmol) and further diluted with DMSO (2.0 mL). The crude solution was purified by HPLC (acetonitrile in water with 0.1% hydrochloric acid) which furnished 18-chloro-4-ethyl-5-[2-(propan-2-yl)phenyl]-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione (6.9 mg, 36%) as a white solid. ESI-MS m/z calc. 548.1285, found 549.49 (M+1)⁺; Retention time: 1.82 minutes; LC method A.

### Example 6: Preparation of Compound 6

### Step 1: 4-Ethyl-5-(2-methoxy-6-methylphenyl)-2-oxa-9λ⁶-thia-6,8,15,23-tetraazatetracyclo[15.3.1.13,7.110,14]tricosa-1(21),3,5,7(23),10(22),11,13,17,19-nonaene-9,9,16-trione (Compound 6)

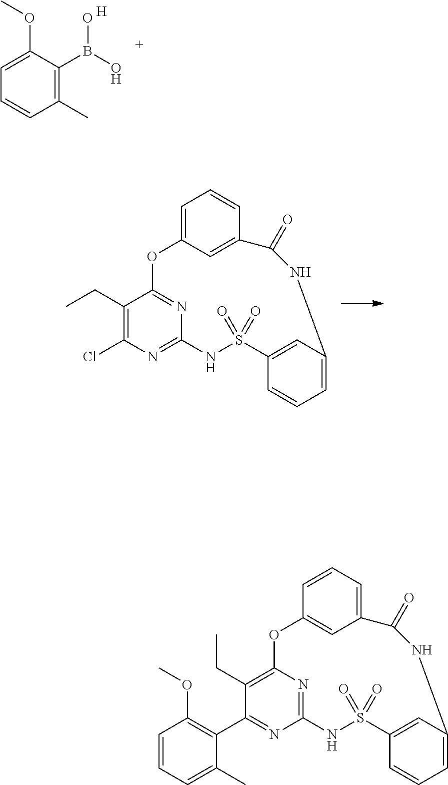

5-Chloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione (30 mg, 0.06545 mmol), (2-methoxy-6-methyl-phenyl)boronic acid (approximately 13.04 mg, 0.07854 mmol), tetrakis(triphenylphosphine)palladium (0) (approximately 6.302 mg, 0.005454 mmol), and 2M aqueous potassium carbonate (approximately 109.0 μL of 2 M, 0.2181 mmol) were combined in dioxane (0.8 mL) and irradiated in the microwave for 30 min at 120° C. The reaction was filtered and purified by reverse phase HPLC utilizing a gradient of 1-99% acetonitrile in 5 mM aqueous HCl to yield 4-ethyl-5-(2-methoxy-6-methylphenyl)-2-oxa-9λ⁶-thia-6,8,15,23-tetraazatetracyclo[15.3.1.13,7.110,14]tricosa-1(21),3,5,7(23),10(22),11,13,17,19-nonaene-9,9,16-trione (13.3 mg, 39%). ESI-MS m/z calc. 516.1467, found 517.3 (M+1)⁺; Retention time: 1.44 minutes; LC method A.

### Example 7: Preparation of Compound 7

### Step 1: 5-Chloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione

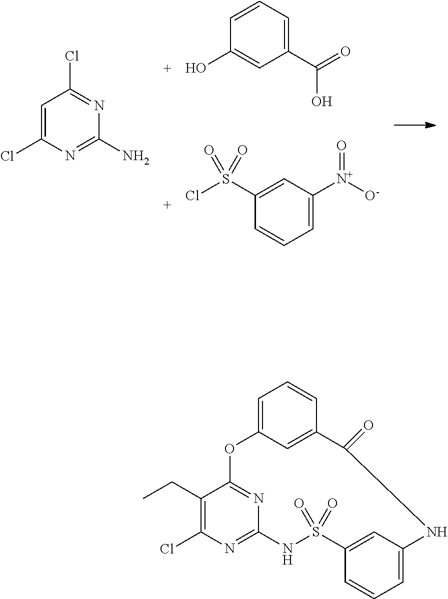 [SMILES: Nc1nc(Cl)cc(Cl)n1 ; O=[N+]([O-])c1cccc(S(=O)(=O)Cl)c1 ; O=C(O)c1cccc(O)c1 ; CCc1c(Cl)nc2nc1Oc1cccc(c1)C(=O)Nc1cccc(c1)S(=O)(=O)N2]

Stage 1: To a solution of 4,6-dichloro-5-ethyl-pyrimidin-2-amine (4.22 g, 21.97 mmol) in DMF (88 mL) at 0° C. was added sodium hydride (3.5 g of 60% w/w, 87.51 mmol and the reaction mixture was stirred at this temperature for 5 minutes, then removed from the cooling bath and stirred at room temperature for 10 minutes. The reaction mixture was cooled to 0° C. and 3-nitrobenzenesulfonyl chloride (9.7 g, 44 mmol) was added slowly over 1 minutes. The reaction mixture was stirred at this temperature for 5 minutes, then removed from the cooling bath and stirred at room temperature for 10 minutes. The reaction mixture was cooled back to 0° C. and quenched with hydrochloric acid (17.3 mL of 37% w/v, 176 mmol), then diluted with a solution of ethyl acetate/hexanes (1:1) and partitioned with a saturated aqueous solution of sodium bicarbonate. The organic layer was separated, and the aqueous layer was further extracted with ethyl acetate/hexanes (1:1, 5×). The combined organic extracts were washed with brine and dried over magnesium sulfate. The solution was filtered, and the filtrate was concentrated in vacuo. The crude residue was purified by flash column chromatography on silica gel (10 to 100% ethyl acetate in hexanes). The starting material co-eluted with the desired product. An impure yellow solid was obtained (6.87 g, ˜30% starting material and 70% desired product).

Stage 2: The impure solid from Stage 1, 3-hydroxybenzoic acid (2.4 g, 17 mmol) and potassium carbonate (12.1 g, 87.6 mmol) were dissolved in NMP (88.0 mL). The solution was heated to 120° C. for 16 h. The reaction was diluted with water and ethyl acetate/hexanes (1:1). The biphasic mixture was acidified with HCl (17.3 mL of 37% w/v, 176 mmol). The organic layer was separated and the aqueous layer was further extracted (4×) with ethyl acetate/hexanes (1:1). The combined organics were washed with brine and dried over magnesium sulfate, filtered, and concentrated in vacuo.

Stage 3: The crude residue from Stage 2 was dissolved in ethanol (89 mL). Iron (6.1 g, 110 mmol) was added followed by hydrochloric acid (5.2 mL of 37% w/v, 53 mmol). The reaction was heated to 70° C. for 1 hour. The reaction mixture was filtered through Celite. It was necessary to filter the filtrate one additional time. The reaction mixture was then concentrated in vacuo.

Stage 4: To the crude residue from Stage 3 dissolved in DMF (42 mL), was added DIPEA (8.5 g, 66 mmol) and HATU (12.5 g, 32.9 mmol). After 30 minutes of stirring at room temperature the reaction was heated to 50° C. for 2 hours. The reaction mixture was diluted with water (150 mL) and then ethyl acetate (500 mL). The precipitate was allowed to settle, and the organic layer was decanted slowly. This was repeated once more and then the organic layers were combined, washed with water and brine and dried over magnesium sulfate. The solution was filtered and concentrated in vacuo. The crude residue was separated by flash column chromatography on silica gel (60% ethyl acetate in hexanes) to afford 5-chloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione (720 mg, 8%) as a yellow solid. ESI-MS m/z calc. 430.05026, found 431.38 (M+1)⁺; Retention time: 0.58 minutes; LC method D.

### Step 2: 4-Ethyl-5-(2-methylpyridin-3-yl)-2-oxa-9λ⁶-thia-6,8,15,23-tetraazatetracyclo[15.3.1.13,7.110,14]tricosa-1(21),3,5,7(23),10(22),11,13,17,19-nonaene-9,9,16-trione (Compound 7)

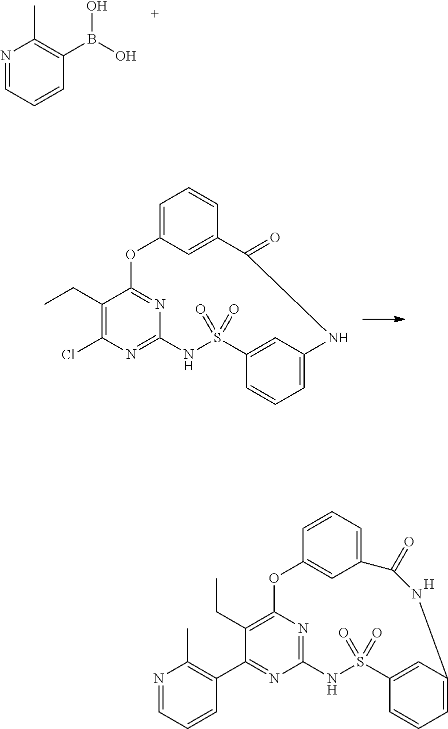

5-Chloro-4-ethyl-2-oxa-9λ⁶-thia-6,8,15,23-tetraazatetracyclo[15.3.1.13,7.110,14]tricosa-1(20),3(23),4,6,10(22),11,13,17(21),18-nonaene-9,9,16-trione (25 mg, 0.05802 mmol), (2-methyl-3-pyridyl)boronic acid (approximately 9.534 mg, 0.06962 mmol), tetrakis(triphenylphosphine)palladium (0) (approximately 6.705 mg, 0.005802 mmol), and 2 M aqueous potassium carbonate (approximately 116.0 μL of 2 M, 0.2321 mmol) were combined in dioxane and irradiated in the microwave for 30 minutes at 120° C. The reaction was filtered and purified by reverse phase HPLC utilizing a gradient of 1-99% acetonitrile in 5 mM aqueous HCl to yield 4-ethyl-5-(2-methylpyridin-3-yl)-2-oxa-9λ⁶-thia-6,8,15,23-tetraazatetracyclo[15.3.1.13,7.110,14]tricosa-1(21),3,5,7(23),10(22),11,13,17,19-nonaene-9,9,16-trione (hydrochloride salt) (13.8 mg, 45%). ESI-MS m/z calc. 487.13144, found 488.0 (M+1)⁺; Retention time: 0.89 minutes; LC method A.

### Example 8: Preparation of Compound 8

### Step 1: 5-(2,6-Dimethylphenyl)-20-methyl-2-oxa-9λ⁶-thia-6,8,15,23-tetraazatetracyclo[15.3.1.13,7.110,14]tricosa-1(21),3(23),4,6,10,12,14(22),17,19-nonaene-9,9,16-trione

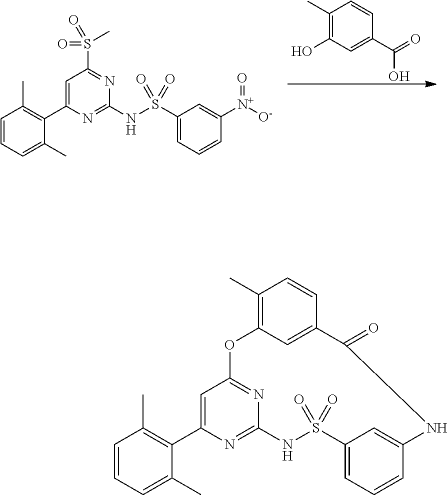 [SMILES: Cc1cccc(C)c1-c1cc(S(C)(=O)=O)nc(NS(=O)(=O)c2cccc([N+](=O)[O-])c2)n1 ; Cc1ccc(C(=O)O)cc1O ; Cc1ccc2cc1Oc1cc(-c3c(C)cccc3C)nc(n1)NS(=O)(=O)c1cccc(c1)NC2=O]

Stage 1: To a 20 mL vial equipped with a magnetic stir bar, N-[4-(2,6-dimethylphenyl)-6-methylsulfonyl-pyrimidin-2-yl]-3-nitro-benzenesulfonamide (149.2 mg, 0.3226 mmol), 3-hydroxy-4-methyl-benzoic acid (151.5 mg, 0.9957 mmol) and N-methylpyrrolidinone (4.0 mL) were added, followed by potassium carbonate (179.2 mg, 1.297 mmol). This solution was stirred at 110° C. for 17 hours. The reaction mixture was then cooled to room temperature, quenched with 1 N HCl (4 mL), and extracted with ethyl acetate (3×4 mL). The combined organic extracts were washed with water (2×4 mL) and saturated aqueous sodium chloride solution (3 mL), then dried over sodium sulfate, filtered, and evaporated in vacuo to give 390 mg of a brown oil. This was purified by silica gel chromatography (12 g of silica, 0 to 40% gradient of ethyl acetate/hexanes) to give 344.2 mg of a white foam, which was not very pure (˜40% pure) but was carried onward.

Stage 2: The product from Stage 1 was dissolved in a mixture of ethanol (2.0 mL) and ethyl acetate (2.0 mL) and transferred to a 10 mL vial equipped with a magnetic stir bar. This solution was purged with a balloon of hydrogen gas for 5 minutes. The cap was briefly removed, and 10% Pd(OH)₂/C (20.4 mg, 0.01453 mmol) was added. This reaction mixture was stirred under a balloon of hydrogen gas at 70° C. for 50 hours. It was cooled to room temperature, filtered through Celite and rinsed with methanol (10 mL), then evaporated in vacuo. Purification by reverse phase HPLC (1-99% acetonitrile in water using HCl as a modifier) provided a white solid, 3-[2-[(3-aminophenyl)sulfonylamino]-6-(2,6-dimethylphenyl)pyrimidin-4-yl]oxy-4-methyl-benzoic acid (hydrochloride salt) (84.0 mg, 48%) ESI-MS m/z calc. 504.14673, found 505.3 (M+1)⁺; Retention time: 1.47 minutes; LC method A.

Stage 3: The product from Stage 2 was dissolved in dimethylformamide (2.0 mL) and transferred to a 10 mL vial equipped with a magnetic stir bar. To this solution were added DIPEA (70 μL, 0.4019 mmol) and HATU (80.4 mg, 0.2115 mmol). This mixture was stirred at room temperature for 5 minutes, after which it was filtered and purified by reverse phase HPLC (1-99% acetonitrile in water using HCl as a modifier) to give 5-(2,6-dimethylphenyl)-20-methyl-2-oxa-9λ⁶-thia-6,8,15,23-tetraazatetracyclo[15.3.1.13,7.110,14]tricosa-1(21),3(23),4,6,10,12,14(22),17,19-nonaene-9,9,16-trione (40.8 mg, 26%) ESI-MS m/z calc. 486.13617, found 487.2 (M+1)⁺; Retention time: 1.46 minutes; LC method A.

### Step 2: 4-Chloro-5-(2,6-dimethylphenyl)-20-methyl-9,9-dioxo-2-oxa-9λ⁶-thia-6,8,15,23-tetrazatetracyclo[15.3.1.13,7.110,14]tricosa-1(20),3,5,7(23),10(22),11,13,17(21),18-nonaen-16-one (Compound 8)

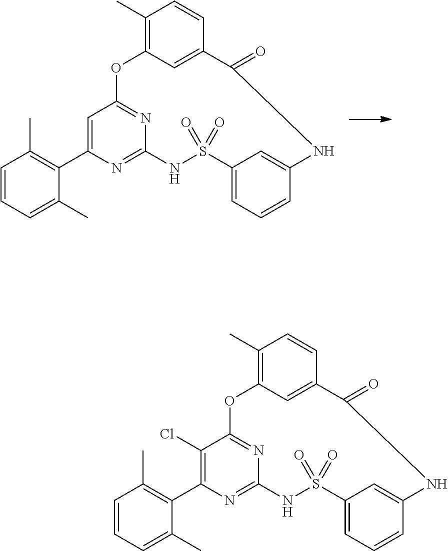 [SMILES: Cc1ccc2cc1Oc1cc(-c3c(C)cccc3C)nc(n1)NS(=O)(=O)c1cccc(c1)NC2=O ; Cc1ccc2cc1Oc1nc(nc(-c3c(C)cccc3C)c1Cl)NS(=O)(=O)c1cccc(c1)NC2=O]

In a 3 mL vial equipped with a magnetic stir bar, 5-(2,6-dimethylphenyl)-20-methyl-2-oxa-9λ⁶-thia-6,8,15,23-tetraazatetracyclo[15.3.1.13,7.110,14]tricosa-1(21),3(23),4,6,10,12,14(22),17,19-nonaene-9,9,16-trione (12.6 mg, 0.02590 mmol) and N-chlorosuccinimide (10.2 mg, 0.07639 mmol) were dissolved in dichloroethane (300 μL) and stirred at 90° C. for 6 hours. After this time, a second batch of N-chlorosuccinimide (10.2 mg, 0.07639 mmol) was added and the reaction mixture was stirred at 90° C. for 6 hours. This solution was cooled to room temperature, diluted with 1:1 methanol: dimethylsulfoxide (500 L), filtered, and purified by reverse phase HPLC (1-70% acetonitrile in water using HCl as a modifier) to give 4-chloro-5-(2,6-dimethylphenyl)-20-methyl-9,9-dioxo-2-oxa-9λ⁶-thia-6,8,15,23-tetrazatetracyclo[15.3.1.13,7.110,14]tricosa-1(20),3,5,7(23),10(22),11,13,17(21),18-nonaen-16-one (8.3 mg, 62%). ¹H NMR (400 MHz, dimethylsulfoxide-d6) δ 12.18 (bs, 1H, D₂O exchangeable), 10.55 (s, 1H, D₂O exchangeable), 7.61 (s, 2H), 7.54 (t, J=7.9 Hz, 1H), 7.41-7.33 (m, 2H), 7.27 (dd, J=8.1, 7.0 Hz, 1H), 7.16 (d, J=7.6 Hz, 2H), 7.00 (s, 1H), 6.84 (t, J=2.0 Hz, 1H), 2.17 (s, 3H), 1.99 (s, 6H). ESI-MS m/z calc. 520.0972, found 521.2 (M+1)⁺; Retention time: 1.7 minutes; LC method A.

### Example 9: Characterization of Compounds 9-30

The compounds in Table 3 were prepared in a manner analogous to that described above using commercially available reagents and intermediates described herein.

TABLE 3

| Compound | | LCMS Rt | Calc. | | LCMS |
|---|---|---|---|---|---|
| Number | Structure | (min) | mass | M + 1 | Method |
| 9 | 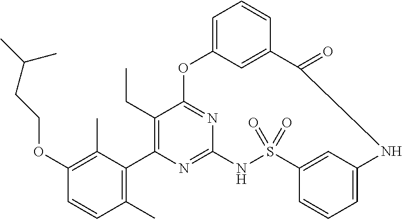 [SMILES: CCc1c2nc(nc1-c1c(C)ccc(OCCC(C)C)c1C)NS(=O)(=O)c1cccc(c1)NC(=O)c1cccc(c1)O2] | 2.08 | 586.225 | 587.33 | A |
| 10 | 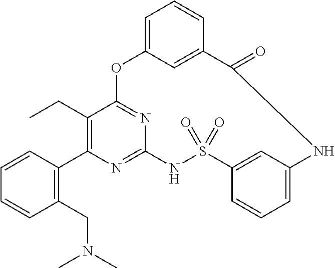 [SMILES: CCc1c2nc(nc1-c1ccccc1CN(C)C)NS(=O)(=O)c1cccc(c1)NC(=O)c1cccc(c1)O2] | 1.25 | 529.178 | 530.51 | A |
| 11 | 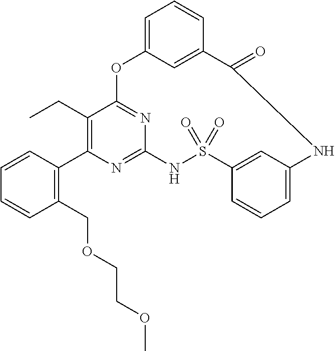 [SMILES: CCc1c2nc(nc1-c1ccccc1COCCOC)NS(=O)(=O)c1cccc(c1)NC(=O)c1cccc(c1)O2] | 1.6 | 560.173 | 561.53 | A |
| 12 | 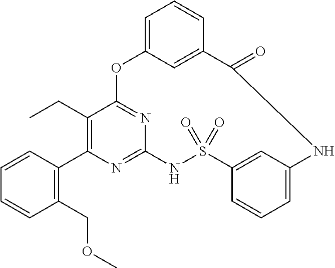 [SMILES: CCc1c2nc(nc1-c1ccccc1COC)NS(=O)(=O)c1cccc(c1)NC(=O)c1cccc(c1)O2] | 1.6 | 516.147 | 517.52 | A |
| 13 | 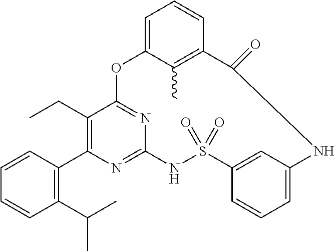 | 1.82 | 528.183 | 529.53 | A |
| 14 | 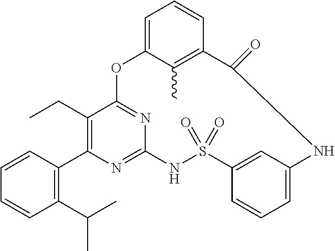 | 1.8 | 528.183 | 529.49 | A |
| 15 | 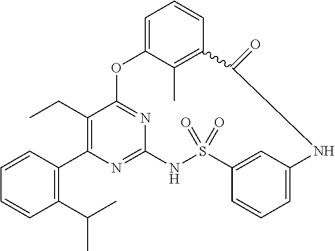 [SMILES: CCc1c2nc(nc1-c1ccccc1C(C)C)NS(=O)(=O)c1cccc(c1)NC(=O)c1cccc(c1C)O2] | 1.78 | 528.183 | 529.53 | A |
| 16 | 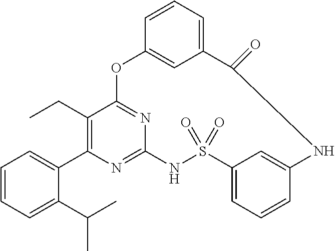 [SMILES: CCc1c2nc(nc1-c1ccccc1C(C)C)NS(=O)(=O)c1cccc(c1)NC(=O)c1cccc(c1)O2] | 1.76 | 514.167 | 515.49 | A |
| 17 | 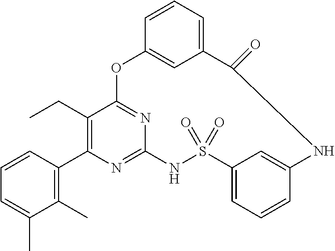 [SMILES: CCc1c2nc(nc1-c1cccc(C)c1C)NS(=O)(=O)c1cccc(c1)NC(=O)c1cccc(c1)O2] | 1.64 | 500.152 | 501.48 | A |
| 18 | 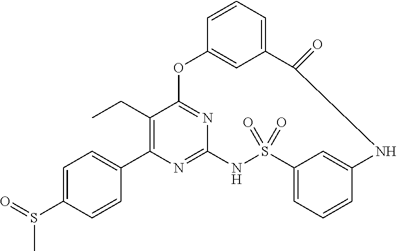 [SMILES: CCc1c2nc(nc1-c1ccc(S(C)=O)cc1)NS(=O)(=O)c1cccc(c1)NC(=O)c1cccc(c1)O2] | 1.14 | 534.103 | 535.41 | A |
| 19 | 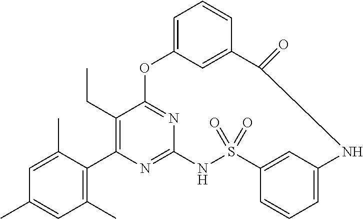 [SMILES: CCc1c2nc(nc1-c1c(C)cc(C)cc1C)NS(=O)(=O)c1cccc(c1)NC(=O)c1cccc(c1)O2] | 1.72 | 514.167 | 515.49 | A |
| 20 | 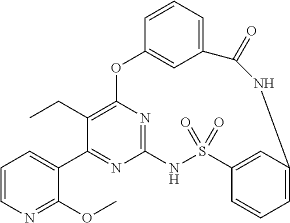 [SMILES: CCc1c2nc(nc1-c1cccnc1OC)NS(=O)(=O)c1cccc(c1)NC(=O)c1cccc(c1)O2] | 1.32 | 503.126 | 504 | A |
| 21 | 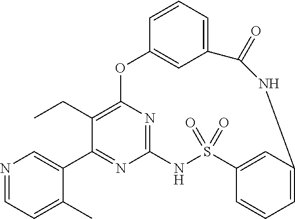 [SMILES: CCc1c2nc(nc1-c1cnccc1C)NS(=O)(=O)c1cccc(c1)NC(=O)c1cccc(c1)O2] | 0.91 | 487.131 | 488 | A |
| 22 | 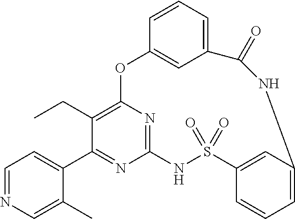 [SMILES: CCc1c2nc(nc1-c1ccncc1C)NS(=O)(=O)c1cccc(c1)NC(=O)c1cccc(c1)O2] | 0.91 | 487.131 | 488 | A |
| 23 | 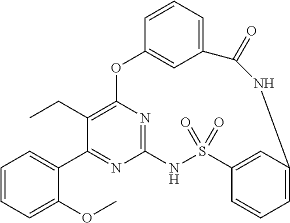 [SMILES: CCc1c2nc(nc1-c1ccccc1OC)NS(=O)(=O)c1cccc(c1)NC(=O)c1cccc(c1)O2] | 1.37 | 502.131 | 503.2 | A |
| 24 | 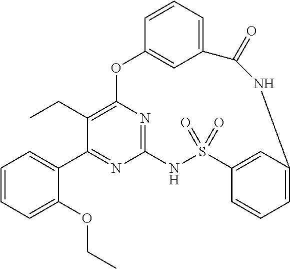 [SMILES: CCOc1ccccc1-c1nc2nc(c1CC)Oc1cccc(c1)C(=O)Nc1cccc(c1)S(=O)(=O)N2] | 1.47 | 516.147 | 517.2 | A |
| 25 | 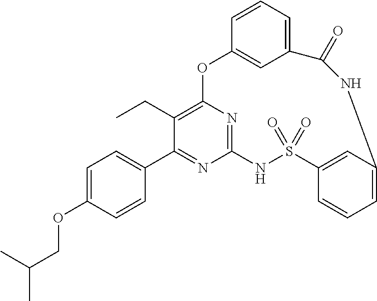 [SMILES: CCc1c2nc(nc1-c1ccc(OCC(C)C)cc1)NS(=O)(=O)c1cccc(c1)NC(=O)c1cccc(c1)O2] | 1.81 | 544.178 | 545.3 | A |
| 26 | 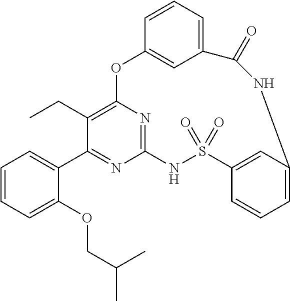 [SMILES: CCc1c2nc(nc1-c1ccccc1OCC(C)C)NS(=O)(=O)c1cccc(c1)NC(=O)c1cccc(c1)O2] | 1.66 | 544.178 | 545.3 | A |
| 27 | 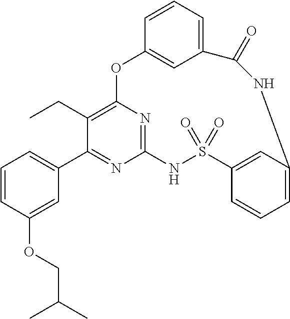 [SMILES: CCc1c2nc(nc1-c1cccc(OCC(C)C)c1)NS(=O)(=O)c1cccc(c1)NC(=O)c1cccc(c1)O2] | 1.83 | 544.178 | 545.3 | A |
| 28 | 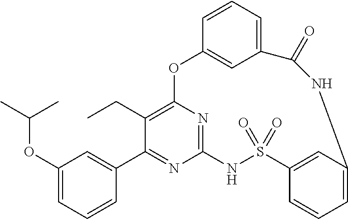 [SMILES: CCc1c2nc(nc1-c1cccc(OC(C)C)c1)NS(=O)(=O)c1cccc(c1)NC(=O)c1cccc(c1)O2] | 1.65 | 530.162 | 531.3 | A |
| 29 | 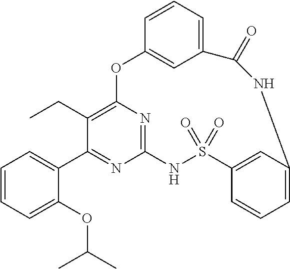 [SMILES: CCc1c2nc(nc1-c1ccccc1OC(C)C)NS(=O)(=O)c1cccc(c1)NC(=O)c1cccc(c1)O2] | 1.55 | 530.162 | 531.3 | A |
| 30 | 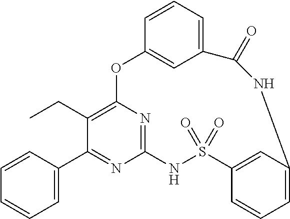 [SMILES: CCc1c2nc(nc1-c1ccccc1)NS(=O)(=O)c1cccc(c1)NC(=O)c1cccc(c1)O2] | 1.39 | 472.121 | 473.3 | A |

### Example 10: Preparation of Compound 31

### Step 1: 6-Methyl-13-(2-methylphenyl)-12-(trifluoromethyl)-10-oxa-17λ⁶-thia-3,6,14,16,23-pentaazatetracyclo[16.3.1.111,15.03,8]tricosa-1(22),11,13,15(23),18,20-hexaene-2,17,17-trione (Compound 31)

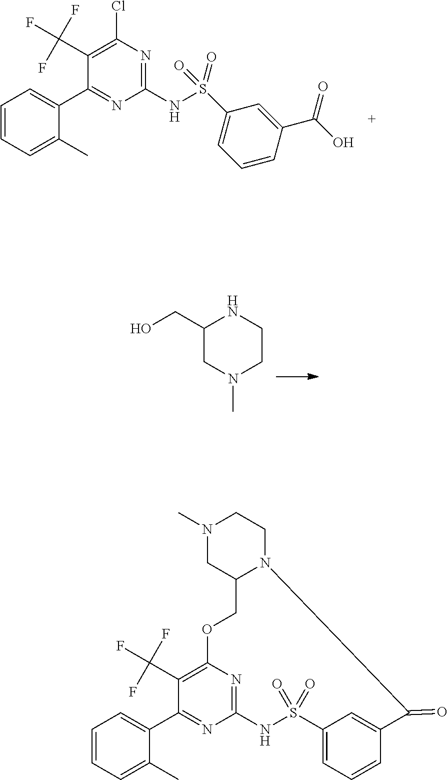

3-[[4-Chloro-6-(o-tolyl)-5-(trifluoromethyl)pyrimidin-2-yl]sulfamoyl]benzoic acid (40 mg, 0.08478 mmol), (4-methylpiperazin-2-yl)methanol (approximately 14.35 mg, 0.1102 mmol), DIC (approximately 13.91 mg, 17.26 μL, 0.1102 mmol), and sodium bicarbonate (approximately 35.61 mg, 0.4239 mmol) were combined in DCM (0.5 mL), and stirred at room temperature for 2 hours. After this time, the reaction mixture was concentrated to a volume of under 0.2 mL, diluted with DMSO and methanol, filtered, and purified by reverse phase HPLC (1-99% ACN in water, HCl) to give the intermediate amide. The product was combined with NaH (approximately 16.95 mg, 0.4239 mmol) in NMP (5 mL) and heated to 70° C. for 4 hours. The reaction mixture was then cooled to room temperature and quenched by slow addition to aqueous ammonium chloride and ethyl acetate. The layers were separated and the aqueous was extracted three additional times with ethyl acetate, and the combined organics were washed with water, then brine, dried over sodium sulfate, and concentrated. The resulting crude material was purified by reverse phase HPLC (1-50% ACN in water, HCl modifier, 15 min run) to give the corresponding 6-methyl-13-(2-methylphenyl)-12-(trifluoromethyl)-10-oxa-17λ⁶-thia-3,6,14,16,23-pentaazatetracyclo[16.3.1.111,15.03,8]tricosa-1(22),11,13,15(23),18,20-hexaene-2,17,17-trione (hydrochloride salt) (2.5 mg, 6%). ESI-MS m/z calc. 547.1501, found 548.3 (M+1)⁺; Retention time: 1.27 minutes; LC method A.

### Example 11: Preparation of Compound 32

### Step 1: tert-Butyl 3-(hydroxymethyl)-4-[2-[1-(trifluoromethyl)cyclopropyl]ethyl]piperazine-1-carboxylate

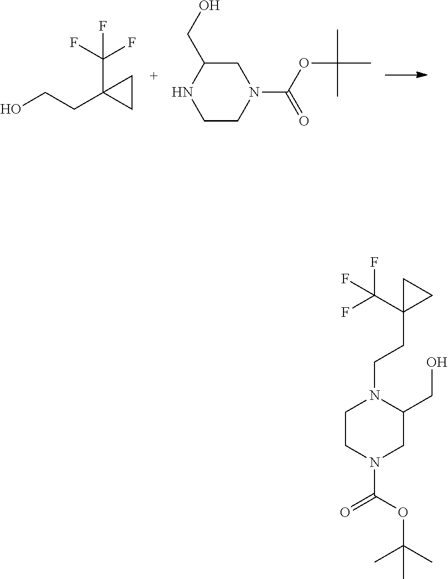 [SMILES: OCCC1(C(F)(F)F)CC1 ; CC(C)(C)OC(=O)N1CCNC(CO)C1 ; CC(C)(C)OC(=O)N1CCN(CCC2(C(F)(F)F)CC2)C(CO)C1]

2-[1-(Trifluoromethyl)cyclopropyl]ethanol (approximately 427.6 mg, 2.774 mmol) was dissolved in 4 mL DCE and Dess-Martin periodinane (approximately 1.236 g, 2.913 mmol) was added and the reaction was stirred at room temperature for 1 h. This reaction mixture was then added to a vial containing tert-butyl 3-(hydroxymethyl)piperazine-1-carboxylate (300 mg, 1.387 mmol) in 2 mL DCE. Acetic acid (500 μL, 8.792 mmol) was then added and the reaction mixture was stirred at room temperature for an additional 2 h. At this point, sodium triacetoxyborohydride (1.3 g, 6.134 mmol) was added and the reaction mixture was allowed to stir an additional 16 h at room temperature. The reaction mixture was then poured into aqueous sodium bicarbonate and extracted 3× ethyl acetate. The combined organics were washed with brine, dried over sodium sulfate, and concentrated. The resulting crude was purified by chromatography on silica gel, eluting with 0-100% ethyl acetate in dichloromethane (not UV active but detected by ELSD), to give tert-butyl 3-(hydroxymethyl)-4-[2-[1-(trifluoromethyl)cyclopropyl]ethyl]piperazine-1-carboxylate (240 mg, 49%) as a colorless oil. ESI-MS m/z calc. 352.1974, found 353.3 (M+1)⁺; Retention time: 0.48 minutes; LC method D.

### Step 2: 12-(2-Methylphenyl)-13-(trifluoromethyl)-18-{2-[1-(trifluoromethyl)cyclopropyl]ethyl}-15-oxa-8λ⁶-thia-1,9,11,18,22-pentaazatetracyclo[15.3.1.13,7.110,14]tricosa-3(23),4,6,10(22),11,13-hexaene-2,8,8-trione (Compound 32)

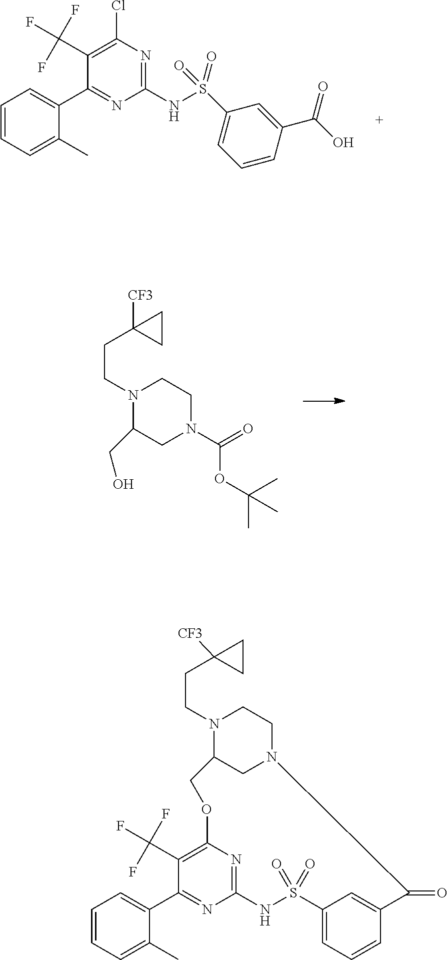 [SMILES: Cc1ccccc1-c1nc(NS(=O)(=O)c2cccc(C(=O)O)c2)nc(Cl)c1C(F)(F)F ; CC(C)(C)OC(=O)N1CCN(CCC2(C(F)(F)F)CC2)C(CO)C1 ; Cc1ccccc1-c1nc2nc(c1C(F)(F)F)OCC1CN(CCN1CCC1(C(F)(F)F)CC1)C(=O)c1cccc(c1)S(=O)(=O)N2]

Stage 1: 3-[[4-Chloro-6-(o-tolyl)-5-(trifluoromethyl)pyrimidin-2-yl]sulfamoyl]benzoic acid (30 mg, 0.06358 mmol), tert-butyl 3-(hydroxymethyl)-4-[2-[1-(trifluoromethyl)cyclopropyl]ethyl]piperazine-1-carboxylate (approximately 33.61 mg, 0.09537 mmol), and NaH (10.1168 mg, 0.4216 mmol) were combined in anhydrous NMP (0.5 mL) and stirred at room temperature for 1 to 6 hours. The reaction mixture was then slowly added to aqueous ammonium chloride and extracted 3× ethyl acetate. The reaction mixture was then quenched with acetic acid, diluted with methanol, filtered, and purified by reverse phase HPLC (1-70% ACN, HCl modifier, 15 min run). The fractions containing product were concentrated to give the desired SNAr product as a foaming solid. The product was dissolved in DCM (0.5 mL), and HCl (0.5 mL of 4 M, 2.000 mmol) was added. After stirring at room temperature for 1 hour the reactions were concentrated to give a white solid. The resulting Boc-deprotected products were used in the next step without further purification.

Stage 2: The product was combined with HATU (13 mg, 0.03419 mmol) in DMF (0.8 mL) and DIPEA (30 μL, 0.1722 mmol) was added. The reaction mixture was stirred at room temperature for 1 hour, then diluted with methanol, filtered and purified by reverse phase HPLC (1-70% ACN, HCl modifier, 15 min run). The fractions containing product were dried to give 12-(2-methylphenyl)-13-(trifluoromethyl)-18-{2-[1-(trifluoromethyl)cyclopropyl]ethyl}-15-oxa-8λ⁶-thia-1,9,11,18,22-pentaazatetracyclo[15.3.1.13,7.110,14]tricosa-3(23),4,6,10(22),11,13-hexaene-2,8,8-trione (hydrochloride salt) (7 mg, 16%) as a white solid. ESI-MS m/z calc. 669.18445, found 670.4 (M+1)⁺; Retention time: 1.69 minutes; LC method A.

### Example 12: Preparation of Compound 33

### Step 1: 13-tert-Butyl-12-(2-methylphenyl)-18-{2-[1-(trifluoromethyl)cyclopropyl]ethyl}-15-oxa-8λ⁶-thia-1,9,11,18,22-pentaazatetracyclo[15.3.1.13,7.110,14]tricosa-3(23),4,6,10(22),11,13-hexaene-2,8,8-trione (Compound 33)

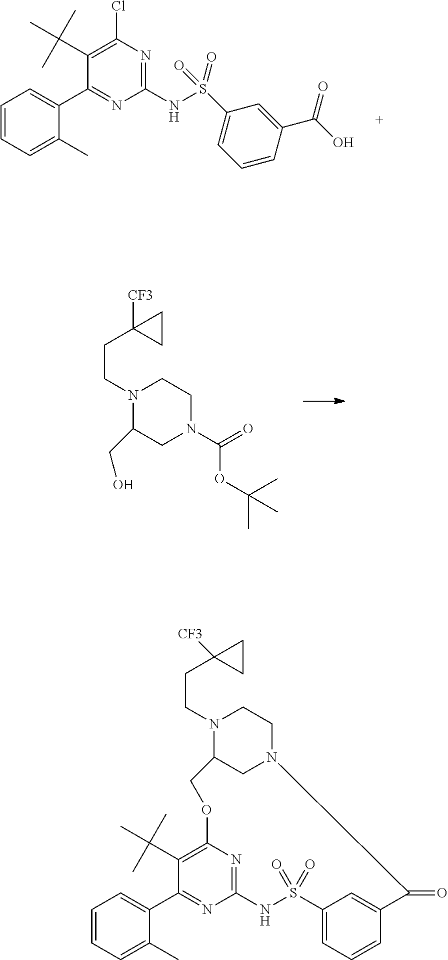 [SMILES: Cc1ccccc1-c1nc(NS(=O)(=O)c2cccc(C(=O)O)c2)nc(Cl)c1C(C)(C)C ; CC(C)(C)OC(=O)N1CCN(CCC2(C(F)(F)F)CC2)C(CO)C1 ; Cc1ccccc1-c1nc2nc(c1C(C)(C)C)OCC1CN(CCN1CCC1(C(F)(F)F)CC1)C(=O)c1cccc(c1)S(=O)(=O)N2]

Stage 1: 3-[[5-tert-butyl-4-chloro-6-(o-tolyl)pyrimidin-2-yl]sulfamoyl]benzoic acid (30 mg, 0.06523 mmol), tert-butyl 3-(hydroxymethyl)-4-[2-[1-(trifluoromethyl)cyclopropyl]ethyl]piperazine-1-carboxylate (approximately 34.48 mg, 0.09784 mmol), and NaH (10.1168 mg, 0.4216 mmol) were combined in anhydrous NMP (0.5 mL) and stirred at room temperature for 1 to 6 hours. The reaction mixture was then slowly added to aqueous ammonium chloride and extracted 3× ethyl acetate. The reaction mixture was then quenched with acetic acid, diluted with methanol, filtered, and purified by reverse phase HPLC (1-70% ACN, HCl modifier, 15 minutes run). The fractions containing product were concentrated to give the desired SNAr product as a foaming solid.

Stage 2: The product was dissolved in DCM (0.5 mL), and HCl (0.5 mL of 4 M, 2.000 mmol) was added. After stirring at room temperature for 1 hour the reactions was concentrated to give a white solid.

Stage 3: The product was combined with HATU (13 mg, 0.03419 mmol) in DMF (0.8 mL) and DIPEA (30 μL, 0.1722 mmol) was added. The reaction mixture was stirred at room temperature for 1 hour, then diluted with methanol, filtered and purified by reverse phase HPLC (1-70% ACN, HCl modifier, 15 minutes run). The fractions containing product was dried overnight to give 13-tert-butyl-12-(2-methylphenyl)-18-{2-[1-(trifluoromethyl)cyclopropyl]ethyl}-15-oxa-8λ⁶-thia-1,9,11,18,22-pentaazatetracyclo[15.3.1.13,7.110,14]tricosa-3(23),4,6,10(22),11,13-hexaene-2,8,8-trione (hydrochloride salt) (3.5 mg, 8%) as a white solid. ESI-MS m/z calc. 657.25964, found 658.5 (M+1)⁺; Retention time: 1.66 minutes; LC method A.

### Example 13: Characterization of Compound 34

The compound in Table 4 was prepared in a manner analogous to that described above using commercially available reagents and intermediates described herein.

TABLE 4

| Compound | | LCMS Rt | Calc. | | LCMS |
|---|---|---|---|---|---|
| number | Structure | (min) | mass | M + 1 | Method |
| 34 | 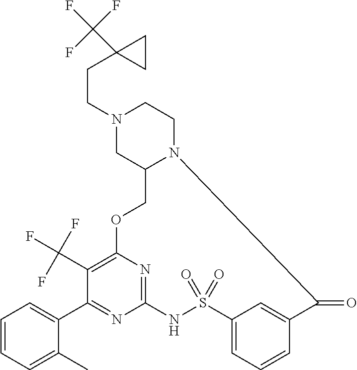 [SMILES: Cc1ccccc1-c1nc2nc(c1C(F)(F)F)OCC1CN(CCC3(C(F)(F)F)CC3)CCN1C(=O)c1cccc(c1)S(=O)(=O)N2] | 1.68 | 669.184 | 670.4 | A |

### Example 14: Preparation of Compound 35

### Step 1: 3-[[4-(2-Aminoethoxy)-6-(2,6-dimethylphenyl)-5-methyl-pyrimidin-2-yl]sulfamoyl]benzoic acid

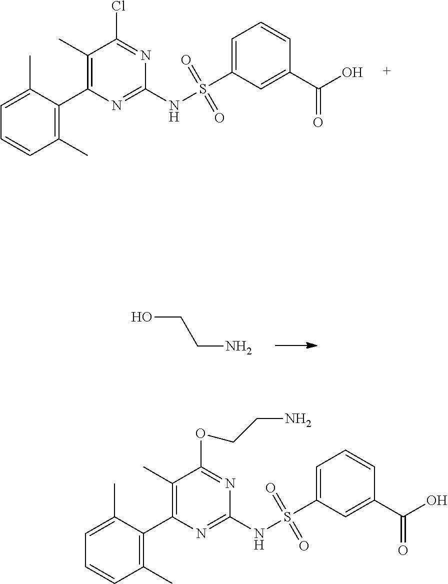

3-[[4-Chloro-6-(2,6-dimethylphenyl)-5-methyl-pyrimidin-2-yl]sulfamoyl]benzoic acid (753 mg, 1.743 mmol) and 2-aminoethanol (115 μL, 1.905 mmol) were combined in THE (3.5 mL) and sodium tert-butoxide (703 mg, 7.315 mmol) was added. The reaction was stirred at room temperature for 10 minutes. The reaction was partitioned between ethyl acetate (10 mL) and a 1 M HCl solution (10 mL). The product crashed out as a white solid and was collected by vacuum filtration. The product was further dried to give 3-[[4-(2-aminoethoxy)-6-(2,6-dimethylphenyl)-5-methyl-pyrimidin-2-yl]sulfamoyl]benzoic acid (hydrochloride salt) (487.6 mg, 57%). ESI-MS m/z calc. 456.14673, found 457.0 (M+1)⁺; Retention time: 0.38 minutes, LC method D.

### Step 2: 6-(2,6-Dimethylphenyl)-7-methyl-9-oxa-2λ⁶-thia-3,5,12,19-tetraazatricyclo[12.3.1.14,8]nonadeca-1(18),4,6,8(19),14,16-hexaene-2,2,13-trione (Compound 35)

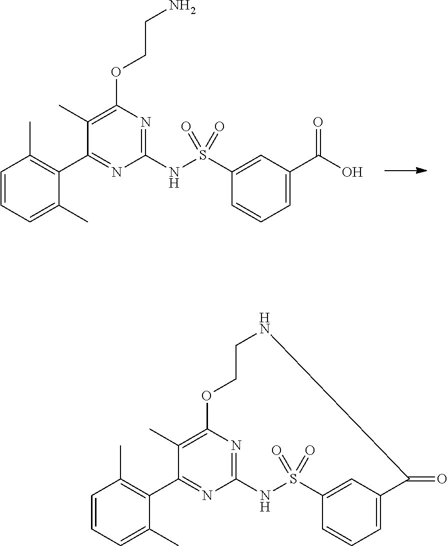 [SMILES: Cc1cccc(C)c1-c1nc(NS(=O)(=O)c2cccc(C(=O)O)c2)nc(OCCN)c1C ; Cc1cccc(C)c1-c1nc2nc(c1C)OCCNC(=O)c1cccc(c1)S(=O)(=O)N2]

3-[[4-(2-Aminoethoxy)-6-(2,6-dimethylphenyl)-5-methyl-pyrimidin-2-yl]sulfamoyl]benzoic acid (hydrochloride salt) (25 mg, 0.05071 mmol) and HATU (19.9 mg, 0.05234 mmol) were dissolved in DMF (1 mL) and DIEA (45 μL, 0.2584 mmol) was added. The reaction mixture was stirred at room temperature for 1 hour. The reaction mixture was filtered and purified by reverse phase HPLC utilizing a gradient of 1-99% acetonitrile in 5 mM aqueous HCl to yield two products: 6-(2,6-Dimethylphenyl)-7-methyl-9-oxa-2λ⁶-thia-3,5,12,19-tetraazatricyclo[12.3.1.14,8]nonadeca-1(18),4,6,8(19),14,16-hexaene-2,2,13-trione (5.4 mg, 23%) as a white solid ESI-MS m/z calc. 438.13617, found 439.1 (M+1)⁺; Retention time: 1.19 minutes (LC method A); and 6-(2,6-dimethylphenyl)-7-methyl-12-oxa-2λ⁶-thia-3,5,9,19-tetraazatricyclo[12.3.1.14,8]nonadeca-1(18),4,6,8(19),14,16-hexaene-2,2,13-trione (5.2 mg, 23%) as a white solid. ESI-MS m/z calc. 438.13617, found 439.1 (M+1)⁺; Retention time: 1.54 minutes (LC method A).

### Example 15: Preparation of Compound 36

### Step 1: 3-[[4-(3-Aminopropoxy)-6-(2,6-dimethylphenyl)-5-methyl-pyrimidin-2-yl]sulfamoyl]benzoic acid

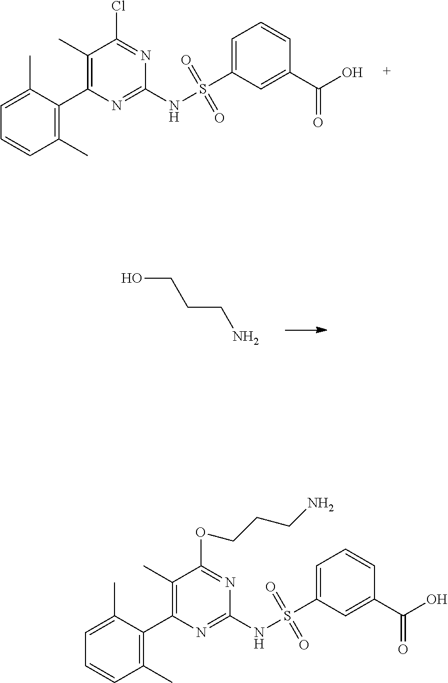 [SMILES: Cc1cccc(C)c1-c1nc(NS(=O)(=O)c2cccc(C(=O)O)c2)nc(Cl)c1C ; NCCCO ; Cc1cccc(C)c1-c1nc(NS(=O)(=O)c2cccc(C(=O)O)c2)nc(OCCCN)c1C]

3-[[4-Chloro-6-(2,6-dimethylphenyl)-5-methyl-pyrimidin-2-yl]sulfamoyl]benzoic acid (745 mg, 1.725 mmol) and 3-aminopropan-1-ol (245 μL, 3.223 mmol) were combined in THE (3 mL) and sodium tert-butoxide (679 mg, 7.065 mmol) was added. The reaction was stirred at room temperature for 10 min. The reaction was partitioned between ethyl acetate (10 mL) and a 1M HCl solution (10 mL). The product crashed out as a white solid and was collected by vacuum filtration. The product was further dried to give 3-[[4-(3-aminopropoxy)-6-(2,6-dimethylphenyl)-5-methyl-pyrimidin-2-yl]sulfamoyl]benzoic acid (hydrochloride salt) (385.6 mg, 44%). ESI-MS m/z calc. 470.16238, found 471.0 (M+1)⁺; Retention time: 0.4 minutes, LC method D.

### Step 2: 6-(2,6-Dimethylphenyl)-7-methyl-9-oxa-2λ⁶-thia-3,5,13,20-tetraazatricyclo[13.3.1.14,8]icosa-1(19),4,6,8(20),15,17-hexaene-2,2,14-trione (Compound 36)

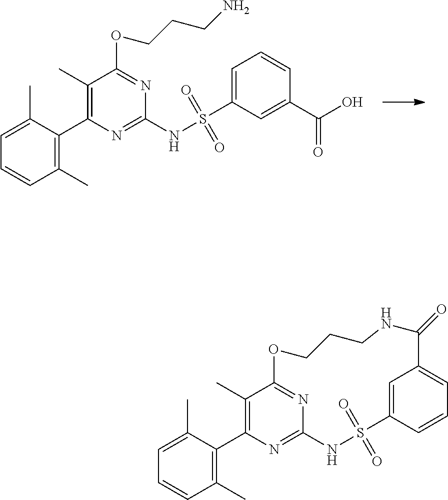 [SMILES: Cc1cccc(C)c1-c1nc(NS(=O)(=O)c2cccc(C(=O)O)c2)nc(OCCCN)c1C ; Cc1cccc(C)c1-c1nc2nc(c1C)OCCCNC(=O)c1cccc(c1)S(=O)(=O)N2]

3-[[4-(3-Aminopropoxy)-6-(2,6-dimethylphenyl)-5-methyl-pyrimidin-2-yl]sulfamoyl]benzoic acid (hydrochloride salt) (24.6 mg, 0.04852 mmol) and HATU (20.3 mg, 0.05339 mmol) were dissolved in DMF (1 mL) and DIEA (45 μL, 0.2584 mmol) was added. The reaction mixture was stirred at room temperature for 1 hour, then filtered and purified by reverse phase HPLC utilizing a gradient of 1-99% acetonitrile in 5 mM aqueous HCl to yield 6-(2,6-dimethylphenyl)-7-methyl-9-oxa-2λ⁶-thia-3,5,13,20-tetraazatricyclo[13.3.1.14,8]icosa-1(19),4,6,8(20),15,17-hexaene-2,2,14-trione (10.2 mg, 46%) as a white solid. ESI-MS m/z calc. 452.15182, found 453.1 (M+1)⁺; Retention time: 1.25 minutes, LC method A.

### Example 16: Characterization of Compounds 37-38

The compounds Table 5 were prepared in a manner analogous to that described above using commercially available reagents and intermediates described herein.

TABLE 5

| Compound | | LCMS Rt | Calc. | | LCMS |
|---|---|---|---|---|---|
| number | Structure | (min) | Mass | M + 1 | Method |
| 37 | 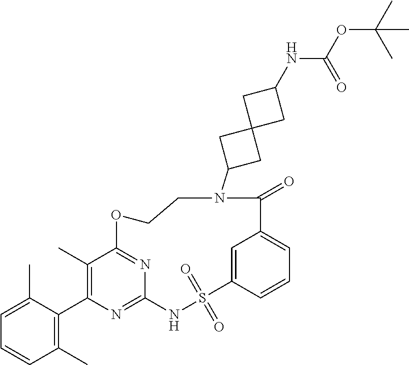 [SMILES: Cc1cccc(C)c1-c1nc2nc(c1C)OCCN(C1CC3(CC(NC(=O)OC(C)(C)C)C3)C1)C(=O)c1cccc(c1)S(=O)(=O)N2] | 1.77 | 647.278 | 648.5 | A |
| 38 | 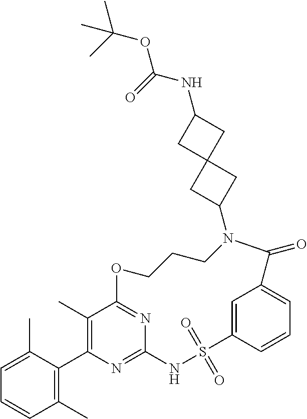 [SMILES: Cc1cccc(C)c1-c1nc2nc(c1C)OCCCN(C1CC3(CC(NC(=O)OC(C)(C)C)C3)C1)C(=O)c1cccc(c1)S(=O)(=O)N2] | 1.81 | 661.293 | 662.7 | A |

### Example 17: Preparation of Compound 39

### Step 1: 3-[(5-tert-Butyl-4,6-dichloro-pyrimidin-2-yl)sulfamoyl]benzoic acid

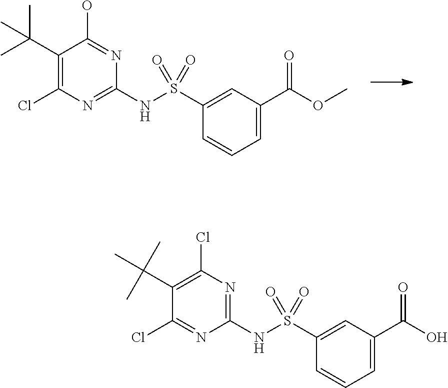

Methyl 3-[(5-tert-butyl-4,6-dichloro-pyrimidin-2-yl)sulfamoyl]benzoate (1.51 g, 3.610 mmol) was dissolved in THF (8.305 mL) and isopropanol (1.661 mL). The solution was cooled to ° C. before the addition of aqueous sodium hydride (approximately 13.72 mL of 1 M, 13.72 mmol). The reaction mixture was allowed to stir at 0° C. for 2 hours. The reaction mixture was then added to aqueous HCl (1 N, 50 mL). The resulting suspension was then extracted with EtOAc (2×50 mL). The combined organic layers were washed with brine (1×75 mL), dried over sodium sulfate, filtered and concentrated under reduced pressure. 3-[(5-tert-Butyl-4,6-dichloro-pyrimidin-2-yl)sulfamoyl]benzoic acid (1.34 g, 920%) was obtained as a white solid. The product was used in the next step without further purification. ESI-MS m/z calc. 403.01602, found 403.9 (M+1)⁺; Retention time: 1.61 minutes; LC method A.

### Step 2: N-(5-tert-Butyl-4,6-dichloro-pyrimidin-2-yl)-3-(4-hydroxy-3,4-dihydro-1H-isoquinoline-2-carbonyl)benzenesulfonamide

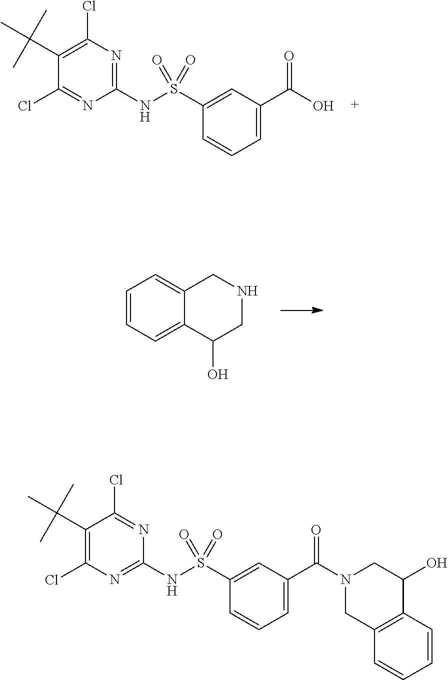 [SMILES: CC(C)(C)c1c(Cl)nc(NS(=O)(=O)c2cccc(C(=O)O)c2)nc1Cl ; OC1CNCc2ccccc21 ; CC(C)(C)c1c(Cl)nc(NS(=O)(=O)c2cccc(C(=O)N3Cc4ccccc4C(O)C3)c2)nc1Cl]

3-[(5-tert-Butyl-4,6-dichloro-pyrimidin-2-yl)sulfamoyl]benzoic acid (381 mg, 0.9424 mmol) was dissolved in DCM. N,N′-Diisopropylmethanediimine (approximately 154.6 mg, 191.8 μL, 1.225 mmol) was added. The mixture was stirred for 15 minutes at room temperature. 1,2,3,4-Tetrahydroisoquinolin-4-ol (approximately 154.7 mg, 1.037 mmol) was then added followed by sodium bicarbonate (approximately 395.8 mg, 4.712 mmol). The final reaction mixture was allowed to stir overnight at room temperature. The reaction mixture was diluted with EtOAc (75 mL) and washed with citric acid (1 M, 75 mL) and brine (75 mL). The organic layer was dried over sodium sulfate, filtered and concentrated under reduced pressure. The crude product was purified by chromatography on a 12 gram silica gel column eluting with a 0-40% EtOAc/hexane gradient over 40 minutes to give N-(5-tert-butyl-4,6-dichloro-pyrimidin-2-yl)-3-(4-hydroxy-3,4-dihydro-1H-isoquinoline-2-carbonyl)benzenesulfonamide (491 mg, 97%). ESI-MS m/z calc. 534.08954, found 1.77 (M+1)⁺; Retention time: 535.2 minutes; LC method A.

### Step 3: 13-tert-Butyl-12-chloro-15-oxa-8λ⁶-thia-1,9,11,25-tetraazapentacyclo[14.7.1.13,7.110,14.017,22]hexacosa-3,5,7(26),10,12,14(25),17(22),18,20-nonaene-2,8,8-trione

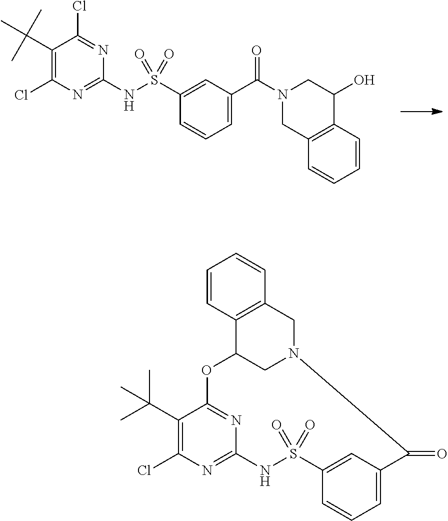 [SMILES: CC(C)(C)c1c(Cl)nc(NS(=O)(=O)c2cccc(C(=O)N3Cc4ccccc4C(O)C3)c2)nc1Cl ; CC(C)(C)c1c(Cl)nc2nc1OC1CN(Cc3ccccc31)C(=O)c1cccc(c1)S(=O)(=O)N2]

Sodium hydride (approximately 177.8 mg, 4.445 mmol) was suspended in NMP (111.1 mL). N-(5-tert-butyl-4,6-dichloro-pyrimidin-2-yl)-3-(4-hydroxy-3,4-dihydro-1H-isoquinoline-2-carbonyl)benzenesulfonamide (476 mg, 0.8890 mmol) was added. The reaction mixture was allowed to stir at 70° C. for 2 hours. The reaction mixture was diluted with EtOAc (50 mL) and washed with aqueous HCl (1 M, 50 mL) and brine (50 mL). The organic layer was dried over sodium sulfate, filtered and concentrated under reduced pressure. The crude product was purified by flash chromatography on a 12 gram silica gel column with a 0-50% EtOAc/hexane gradient to give 13-tert-butyl-12-chloro-15-oxa-8λ⁶-thia-1,9,11,25-tetraazapentacyclo[14.7.1.13,7.110,14.017,22]hexacosa-3,5,7(26),10,12,14(25),17(22),18,20-nonaene-2,8,8-trione (175 mg, 39%). ESI-MS m/z calc. 498.11285, found 499.2 (M+1)⁺; Retention time: 1.94 minutes; LC method A.

### Step 4: 13-tert-Butyl-12-(o-tolyl)-8,8-dioxo-15-oxa-8λ⁶-thia-1,9,11,25-tetrazapentacyclo[14.7.1.13,7.110,14.017,22]hexacosa-3,5,7(26),10(25),11,13,17(22),18,20-nonaen-2-one (Compound 39)

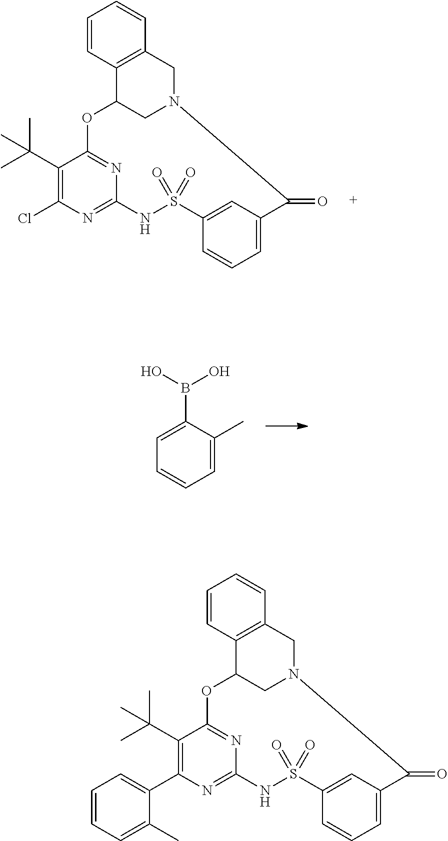

A solution of 13-tert-butyl-12-chloro-15-oxa-8λ⁶-thia-1,9,11,25-tetraazapentacyclo[14.7.1.13,7.110,14.017,22]hexacosa-3,5,7(26),10,12,14(25),17(22),18,20-nonaene-2,8,8-trione (25 mg, 0.05010 mmol) in dioxane (0.5 mL) was added to o-tolylboronic acid (approximately 8.174 mg, 0.06012 mmol). Aqueous potassium carbonate (approximately 100.2 μL of 2 M, 0.2004 mmol) was added followed by tetrakis(triphenylphosphane)palladium(0) (approximately 5.789 mg, 0.005010 mmol) under nitrogen. The reaction vessel was sealed and stirred in an oil bath at 120° C. for 30 minutes. The product was purified by reverse phase HPLC using a Luna Cis (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM HCl acid modifier). Mobile phase B=acetonitrile. Flow rate=35 mL/min, injection volume=950 μL, and column temperature=25° C. 13-tert-Butyl-12-(o-tolyl)-8,8-dioxo-15-oxa-8λ⁶-thia-1,9,11,25-tetrazapentacyclo-[14.7.1.13,7.110,14.017,22]hexacosa-3,5,7(26),10(25),11,13,17(22),18,20-nonaen-2-one (4.1 mg, 14%) was obtained. ESI-MS m/z calc. 554.1988, found 555.3 (M+1)⁺; Retention time: 1.86 minutes; LC method A.

### Example 18: Preparation of Compound 40

### Step 1: tert-Butyl 4-[6-chloro-2-[(3-methoxycarbonylphenyl)sulfonylamino]-5-(trifluoromethyl)pyrimidin-4-yl]oxy-3,4-dihydro-1H-isoquinoline-2-carboxylate

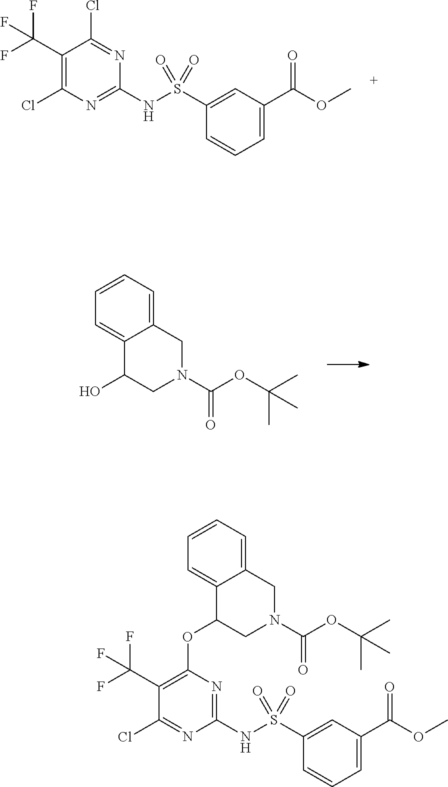 [SMILES: COC(=O)c1cccc(S(=O)(=O)Nc2nc(Cl)c(C(F)(F)F)c(Cl)n2)c1 ; CC(C)(C)OC(=O)N1Cc2ccccc2C(O)C1 ; COC(=O)c1cccc(S(=O)(=O)Nc2nc(Cl)c(C(F)(F)F)c(OC3CN(C(=O)OC(C)(C)C)Cc4ccccc43)n2)c1]

Methyl 3-[[4,6-dichloro-5-(trifluoromethyl)pyrimidin-2-yl]sulfamoyl]benzoate (750 mg, 1.743 mmol) and tert-butyl 4-hydroxy-3,4-dihydro-1H-isoquinoline-2-carboxylate (approximately 651.7 mg, 2.614 mmol) were combined and dissolved in DMSO (7.5 mL). Solid cesium carbonate (approximately 1.704 g, 5.229 mmol) was added at room temperature. After stirring for 10 minutes, the reaction mixture was transferred to aqueous HCl (1 M, 50 mL). The mixture was extracted with EtOAc (1×50 mL). The organic layer was washed with brine (1×50 mL), dried over sodium sulfate, filtered and concentrated under reduced pressure. tert-Butyl 4-[6-chloro-2-[(3-methoxycarbonylphenyl)sulfonylamino]-5-(trifluoromethyl)pyrimidin-4-yl]oxy-3,4-dihydro-1H-isoquinoline-2-carboxylate (1.52 g, 136%) was obtained. ESI-MS m/z calc. 642.1163, found 643.5 (M+1)⁺; Retention time: 2.12 minutes, LC method A.

### Step 2: Methyl 3-[[4-chloro-6-(1,2,3,4-tetrahydroisoquinolin-4-yloxy)-5-(trifluoromethyl)pyrimidin-2-yl]sulfamoyl]benzoate

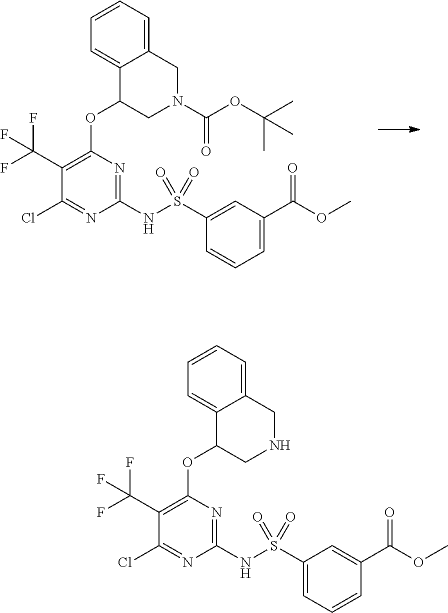 [SMILES: COC(=O)c1cccc(S(=O)(=O)Nc2nc(Cl)c(C(F)(F)F)c(OC3CN(C(=O)OC(C)(C)C)Cc4ccccc43)n2)c1 ; COC(=O)c1cccc(S(=O)(=O)Nc2nc(Cl)c(C(F)(F)F)c(OC3CNCc4ccccc43)n2)c1]

tert-Butyl 4-[6-chloro-2-[(3-methoxycarbonylphenyl)sulfonylamino]-5-(trifluoromethyl)pyrimidin-4-yl]oxy-3,4-dihydro-1H-isoquinoline-2-carboxylate (1.52 g, 2.364 mmol) was dissolved in a prepared solution of TFA (2.5 mL, 32.45 mmol) in DCM (7.5 mL). The reaction mixture was allowed to stir at room temperature for 30 minutes. Solvents were then removed under reduced pressure. The remaining oil was taken up in EtOAc (50 mL) and washed with HCl (50 mL) and brine (50 mL). The organic layer was dried over sodium sulfate, filtered and concentrated under reduced pressure to give methyl 3-[[4-chloro-6-(1,2,3,4-tetrahydroisoquinolin-4-yloxy)-5-(trifluoromethyl)pyrimidin-2-yl]sulfamoyl]benzoate (1.30 g, 101%) was obtained. ESI-MS m/z calc. 542.06384, found 543.1 (M+1)⁺; Retention time: 1.32 minutes; LC method A.

### Step 3: 3-[[4-Chloro-6-(1,2,3,4-tetrahydroisoquinolin-4-yloxy)-5-(trifluoromethyl)pyrimidin-2-yl]sulfamoyl]benzoic acid

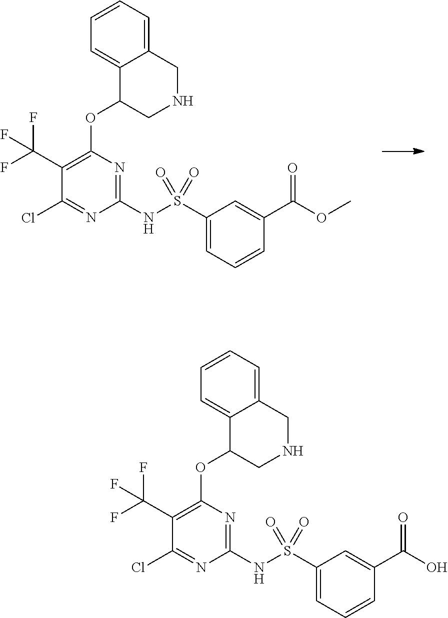 [SMILES: COC(=O)c1cccc(S(=O)(=O)Nc2nc(Cl)c(C(F)(F)F)c(OC3CNCc4ccccc43)n2)c1 ; O=C(O)c1cccc(S(=O)(=O)Nc2nc(Cl)c(C(F)(F)F)c(OC3CNCc4ccccc43)n2)c1]

Methyl 3-[[4-chloro-6-(1,2,3,4-tetrahydroisoquinolin-4-yloxy)-5-(trifluoromethyl)pyrimidin-2-yl]sulfamoyl]benzoate (1.30 g, 2.394 mmol) was dissolved in tetrahydrofuran (6.5 mL) and methanol (1.3 mL). Aqueous sodium hydroxide (9.6 mL of 1 M, 9.600 mmol) was added. The reaction mixture was allowed to stir at room temperature for 30 minutes. The reaction mixture was diluted with EtOAc (50 mL) and washed with HCl (1 N, 1×50 mL) and brine (50 mL). The organic layer was dried over sodium sulfate, filtered and concentrated under reduced pressure. The product was purified by chromatography on a 24 gram silica gel column eluting with a 0-10% MeOH/DCM gradient to give 3-[[4-chloro-6-(1,2,3,4-tetrahydroisoquinolin-4-yloxy)-5-(trifluoromethyl)pyrimidin-2-yl]sulfamoyl]benzoic acid (263 mg, 21%) as a white solid. ESI-MS m/z calc. 528.0482, found 529.2 (M+1)⁺; Retention time: 1.16 minutes, LC method A.

### Step 4: 12-Chloro-13-(trifluoromethyl)-15-oxa-8λ⁶-thia-1,9,11,25-tetraazapentacyclo[14.7.1.13,7.110,14.017,22]hexacosa-3(26),4,6,10(25),11,13,17,19,21-nonaene-2,8,8-trione

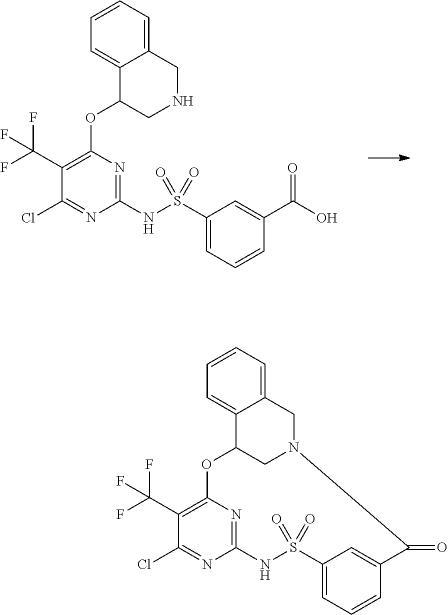 [SMILES: O=C(O)c1cccc(S(=O)(=O)Nc2nc(Cl)c(C(F)(F)F)c(OC3CNCc4ccccc43)n2)c1 ; O=C1c2cccc(c2)S(=O)(=O)Nc2nc(Cl)c(C(F)(F)F)c(n2)OC2CN1Cc1ccccc12]

3-[[4-Chloro-6-(1,2,3,4-tetrahydroisoquinolin-4-yloxy)-5-(trifluoromethyl)pyrimidin-2-yl]sulfamoyl]benzoic acid (263 mg, 0.4973 mmol) was dissolved/suspended in dichloromethane (75 mL). DIC (100 μL, 0.6387 mmol) was added. The reaction mixture was allowed to stir at room temperature overnight. The reaction mixture was concentrated under reduced pressure to −20 mL, diluted with EtOAc (50 mL) and washed with HCl (1 N, 2×50 mL) and brine (50 mL). The organic layer was dried over sodium sulfate, filtered and concentrated under reduced pressure to give 12-chloro-13-(trifluoromethyl)-15-oxa-8λ⁶-thia-1,9,11,25-tetraazapentacyclo[14.7.1.13,7.110,14.017,22]hexacosa-3(26),4,6,10(25),11,13,17,19,21-nonaene-2,8,8-trione (251 mg, 99%) as a light brown-orange solid. ESI-MS m/z calc. 510.03763, found 511.0 (M+1)⁺; Retention time: 1.77 minutes, LC method A.

### Step 5: 12-(2-Methyl-3-pyridyl)-8,8-dioxo-13-(trifluoromethyl)-15-oxa-8λ⁶-thia-1,9,11,25-tetrazapentacyclo[14.7.1.13,7.110,14.017,22]hexacosa-3,5,7(26),10(25),11,13,17(22),18,20-nonaen-2-one (Compound 40)

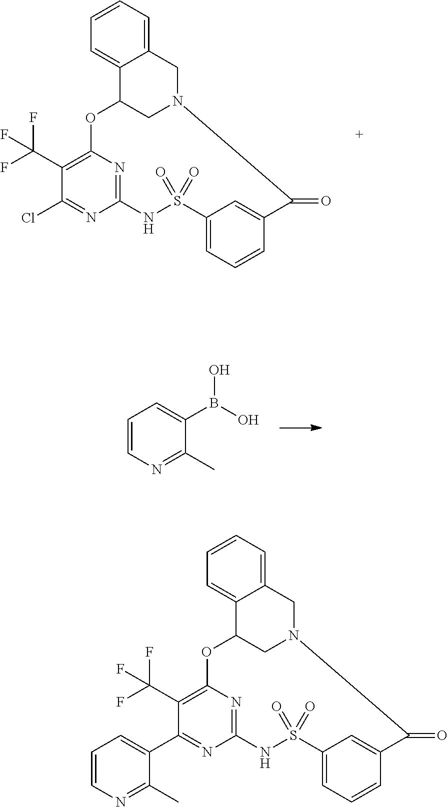

A solution of 12-chloro-13-(trifluoromethyl)-15-oxa-8λ⁶-thia-1,9,11,25-tetraazapentacyclo[14.7.1.13,7.110,14.017,22]hexacosa-3(26),4,6,10(25),11,13,17,19,21-nonaene-2,8,8-trione (30 mg, 0.05872 mmol) in dioxane (0.5 mL) was added to (2-methyl-3-pyridyl)boronic acid (approximately 8.041 mg, 0.05872 mmol). Aqueous potassium carbonate (approximately 97.85 μL of 2 M, 0.1957 mmol) was added followed by tetrakis(triphenylphosphane)palladium(0) (approximately 5.654 mg, 0.004893 mmol) under nitrogen. The reaction vessel was sealed and stirred at 120° C. for 30 minutes. It was diluted with DMSO and purified by reverse-phase HPLC using a Luna Cis (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C., to give 12-(2-methyl-3-pyridyl)-8,8-dioxo-13-(trifluoromethyl)-15-oxa-8λ⁶-thia-1,9,11,25-tetrazapentacyclo[14.7.1.13,7.110,14.017,22]hexacosa-3,5,7(26),10(25),11,13,17(22),18,20-nonaen-2-one (5.3 mg, 16%). ESI-MS m/z calc. 567.11884, found 568.2 (M+1)⁺; Retention time: 1.32 minutes; LC method A.

### Example 19: Characterization of Compounds 41-46

The compounds in Table 6 were prepared in a manner analogous to that described above using commercially available reagents and intermediates described herein.

TABLE 6

| Compound | | LCMS Rt | Calc. | | LCMS |
|---|---|---|---|---|---|
| Number | Structure | (min) | Mass | M + 1 | Method |
| 41 | 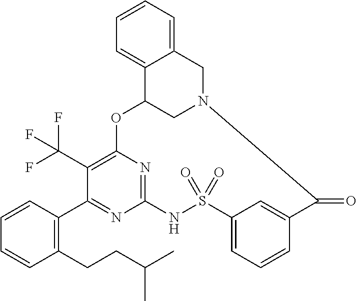 [SMILES: CC(C)CCc1ccccc1-c1nc2nc(c1C(F)(F)F)OC1CN(Cc3ccccc31)C(=O)c1cccc(c1)S(=O)(=O)N2] | 2.2 | 622.186 | 623.3 | A |
| 42 | 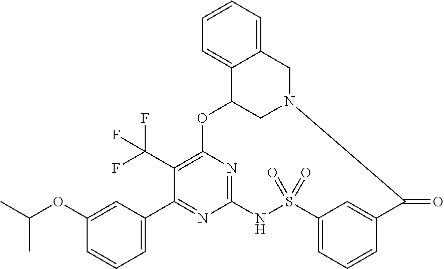 [SMILES: CC(C)Oc1cccc(-c2nc3nc(c2C(F)(F)F)OC2CN(Cc4ccccc42)C(=O)c2cccc(c2)S(=O)(=O)N3)c1] | 2.03 | 610.15 | 611.2 | A |
| 43 | 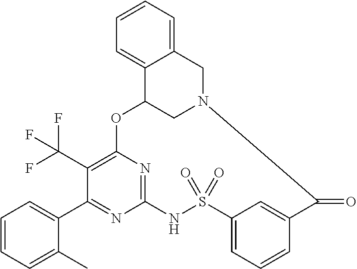 [SMILES: Cc1ccccc1-c1nc2nc(c1C(F)(F)F)OC1CN(Cc3ccccc31)C(=O)c1cccc(c1)S(=O)(=O)N2] | 1.85 | 566.124 | 567.2 | A |
| 44 | 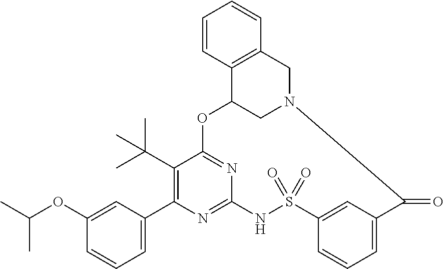 [SMILES: CC(C)Oc1cccc(-c2nc3nc(c2C(C)(C)C)OC2CN(Cc4ccccc42)C(=O)c2cccc(c2)S(=O)(=O)N3)c1] | 2.02 | 598.225 | 599.2 | A |
| 45 | 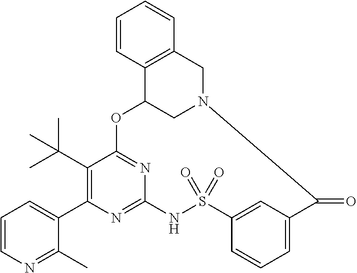 [SMILES: Cc1ncccc1-c1nc2nc(c1C(C)(C)C)OC1CN(Cc3ccccc31)C(=O)c1cccc(c1)S(=O)(=O)N2] | 1.32 | 555.194 | 556.3 | A |
| 46 | 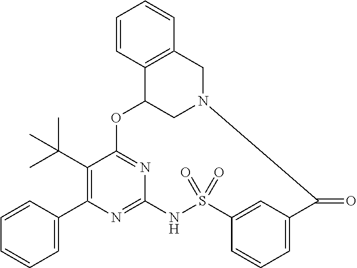 [SMILES: CC(C)(C)c1c2nc(nc1-c1ccccc1)NS(=O)(=O)c1cccc(c1)C(=O)N1Cc3ccccc3C(C1)O2] | 1.8 | 540.183 | 541.2 | A |

### Example 20: Preparation of Compound 47 and Compound 48

### Step 1: 3-[[4-[(2R)-2-Amino-4-methyl-pentoxy]-6-(2,6-dimethylphenyl)-5-methyl-pyrimidin-2-yl]sulfamoyl]benzoic acid

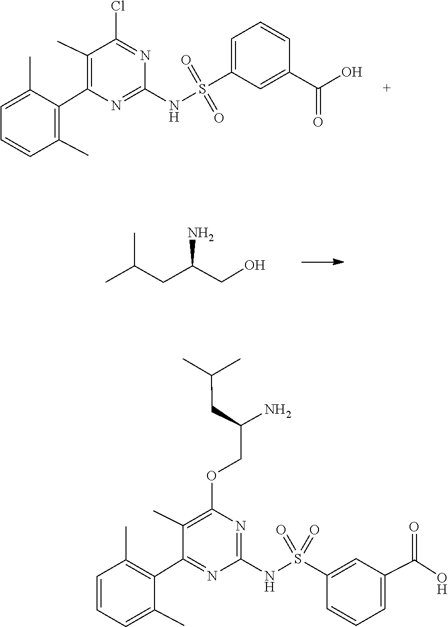

3-[[4-Chloro-6-(2,6-dimethylphenyl)-5-methyl-pyrimidin-2-yl]sulfamoyl]benzoic acid (1.35 g, 3.126 mmol) and (2R)-2-amino-4-methyl-pentan-1-ol (402 mg, 3.430 mmol) were combined in THF (8 mL) at room temperature, and sodium tert-butoxide (1.225 g, 12.75 mmol) was added. The reaction mixture was stirred for 5 minutes. The reaction mixture was then added to a separatory funnel containing 1 M HCl and ethyl acetate. The layers were separated and the aqueous was extracted 3 additional times with ethyl acetate. The combined organics were washed with brine, dried over sodium sulfate, filtered and concentrated to give a white solid, 3-[[4-[(2R)-2-amino-4-methyl-pentoxy]-6-(2,6-dimethylphenyl)-5-methyl-pyrimidin-2-yl]sulfamoyl]benzoic acid (hydrochloride salt) (1.628 g, 95%) ESI-MS m/z calc. 512.20935, found 513.3 (M+1)⁺; Retention time: 0.45 minutes, LC method D.

### Step 2: (11R)-6-(2,6-dimethylphenyl)-11-isobutyl-12-(3-isopropoxycyclobutyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 47) and (11R)-6-(2,6-dimethylphenyl)-11-isobutyl-12-(3-isopropoxycyclobutyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 48)

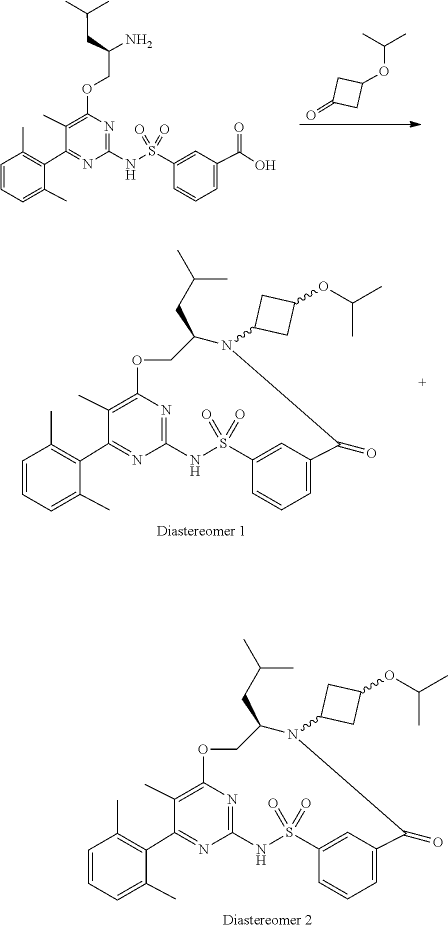

3-[[4-[(2R)-2-Amino-4-methyl-pentoxy]-6-(2,6-dimethylphenyl)-5-methyl-pyrimidin-2-yl]sulfamoyl]benzoic acid (hydrochloride salt) (80 mg, 0.1457 mmol) was combined with 3-isopropoxycyclobutanone (28 mg, 0.2185 mmol) in DCM (0.3 mL) at room temperature and, after stirring for 10 minutes sodium triacetoxyborohydride (93 mg, 0.4388 mmol) was added. The reaction was stirred for 1 hour at room temperature, then a second portion of sodium triacetoxyborohydride (93 mg, 0.4388 mmol) was added and the reaction was stirred for an additional hour. The reaction mixture was then partitioned between 1M HCl and ethyl acetate. The layers were separated and the aqueous was extracted an additional 3× with ethyl acetate. The combined organics were washed with brine, dried over sodium sulfate, and concentrated then in the second step without additional purification (mixture of syn and anti cyclobutane isomers).

This mixture of products was combined with HATU (83 mg, 0.2183 mmol) in DMF (6 mL) and DIPEA (150 μL, 0.8612 mmol) was added. The reaction was at room temperature. The reaction mixture was then concentrated to a volume of less than 1 mL then diluted with methanol, filtered, and purified by reverse phase HPLC (1-99% ACN in water, HCl modifier, 30 min run) to give the cyclization product as a mix of syn and anti cyclobutane isomers. The mixture was subjected to chiral SFC purification using a ChiralPak AS-H (250×10 mm) column with a mobile phase of 12% methanol (no modifier), and 88% CO₂ and a flowrate of 10 mL/min with an approximately 23 mg/mL initial concentration in methanol to give as single stereoisomers (syn/anti undetermined): Peak 1, diastereomer 1, (11R)-6-(2,6-dimethylphenyl)-11-isobutyl-12-(3-isopropoxycyclobutyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (8.2 mg, 9%), ESI-MS m/z calc. 606.2876, found 607.7 (M+1)⁺; Retention time: 2.04 minutes; LC method A; and diastereomer 2, peak 2 (11R)-6-(2,6-dimethylphenyl)-11-isobutyl-12-(3-isopropoxycyclobutyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (8.2 mg, 9%), ESI-MS m/z calc. 606.2876, found 607.5 (M+1)⁺; Retention time: 2.02 minutes; LC method A.

### Example 21: Preparation of Compound 49

### Step 1: 3-[[4-[(2R)-2-Amino-4,4-dimethyl-pentoxy]-6-(2,6-dimethylphenyl)-5-methyl-pyrimidin-2-yl]sulfamoyl]benzoic acid

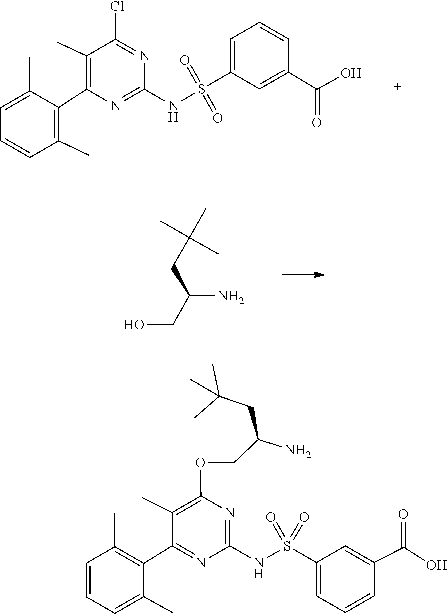

In a 100 mL flask, 3-[[4-chloro-6-(2,6-dimethylphenyl)-5-methyl-pyrimidin-2-yl]sulfamoyl]benzoic acid (872 mg, 2.019 mmol) and (2R)-2-amino-4,4-dimethyl-pentan-1-ol (hydrochloride salt) (341 mg, 2.034 mmol) were charged under nitrogen with anhydrous THF (5 mL) (suspension). Sodium tert-butoxide (770 mg, 8.012 mmol) was added (slight exotherm). The solids slowly dissolved. After 4 hours, another 118 mg of amino alcohol was added, and the mixture was stirred for 1.5 hours. The mixture was partitioned between ethyl acetate (30 mL) and aqueous 1M HCl (30 mL). After separation, the aqueous phase was further extracted with EtOAc (30 mL). Brine was added to the aqueous phase that still contained large amounts of product and it was extracted with ethyl acetate (30 mL). The combined extracts were dried over sodium sulfate and the solvents evaporated to give crude 3-[[4-[(2R)-2-amino-4,4-dimethyl-pentoxy]-6-(2,6-dimethylphenyl)-5-methyl-pyrimidin-2-yl]sulfamoyl]benzoic acid (hydrochloride salt) (1.12 g, 99%). ESI-MS m/z calc. 526.225, found 527.33 (M+1)⁺; Retention time: 0.45 minutes; LC method D.

### Step 2: (11R)-6-(2,6-Dimethylphenyl)-11-(2,2-dimethylpropyl)-7-methyl-12-{[5-(morpholin-4-yl)pyridin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (Compound 49)

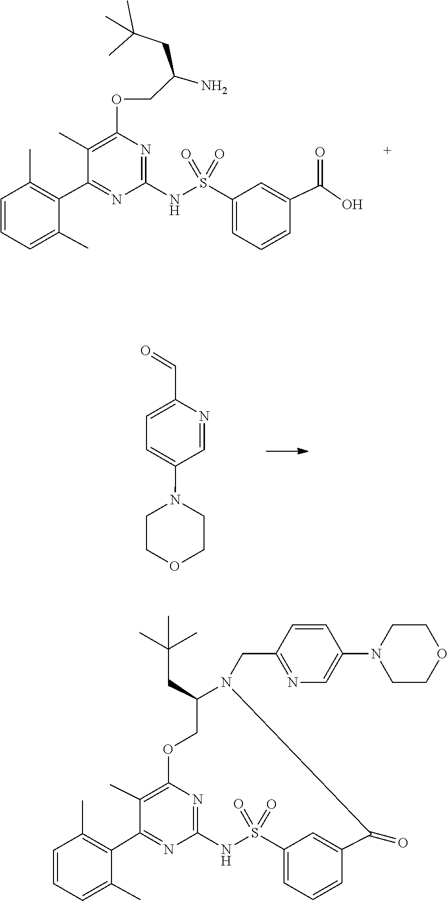 [SMILES: Cc1cccc(C)c1-c1nc(NS(=O)(=O)c2cccc(C(=O)O)c2)nc(OC[C@H](N)CC(C)(C)C)c1C ; O=Cc1ccc(N2CCOCC2)cn1 ; Cc1cccc(C)c1-c1nc2nc(c1C)OC[C@@H](CC(C)(C)C)N(Cc1ccc(N3CCOCC3)cn1)C(=O)c1cccc(c1)S(=O)(=O)N2]

A 4 mL vial was charged with 3-[[4-[(2R)-2-amino-4,4-dimethyl-pentoxy]-6-(2,6-dimethylphenyl)-5-methyl-pyrimidin-2-yl]sulfamoyl]benzoic acid (hydrochloride salt) (94 mg, 0.1669 mmol), 5-morpholinopyridine-2-carbaldehyde (40 mg, 0.2081 mmol) and DCM (400 μL). The mixture was stirred at room temperature for 20 min. Sodium triacetoxyborohydride (50 mg, 0.2359 mmol) was added, the vial was purged with nitrogen, capped and the mixture was stirred at room temperature for 1h. More sodium triacetoxyborohydride (110 mg, 0.5190 mmol) was added and the mixture was stirred at room temperature for 6 h. The mixture was stored in the freezer overnight. The reaction was quenched with a minimum amount of 1N aqueous HCl. Methanol and DMSO were added. The solution was filtered through syringe filter disc and purified by reverse phase preparative HPLC (C₁₈ column) using a gradient (1-99% over 15 min) of acetonitrile in aqueous 5 mM HCl to give 3-[[4-[(2R)-4,4-dimethyl-2-[(5-morpholino-2-pyridyl)methylamino]pentoxy]-6-(2,6-dimethylphenyl)-5-methyl-pyrimidin-2-yl]sulfamoyl]benzoic acid (hydrochloride salt) (47.6 mg, 39%) as a tan solid. ESI-MS m/z calc. 702.31995, found 703.54 (M+1)⁺; Retention time: 1.28 minutes (LC method A).

The product was combined under nitrogen in a 4 mL vial with CDMT (57 mg, 0.3247 mmol) and anhydrous DMF (3 mL). The vial was cooled down in an ice-water bath, 4-methylmorpholine (0.09 mL, 0.8186 mmol) was added and the mixture was stirred in the cooling bath that was allowed to warm to room temperature for 15 h. The crude solution was diluted with methanol, filtered through syringe filter disc and purified by reverse phase preparative HPLC (C₁₈ column) using a gradient (1-99% over 15 min) of acetonitrile in aqueous 5 mM HCl. The product was purified a second time using a 30 min gradient. Evaporation gave (11R)-6-(2,6-dimethylphenyl)-11-(2,2-dimethylpropyl)-7-methyl-12-{[5-(morpholin-4-yl)pyridin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt) (6.7 mg, 5%) as an off-white solid. ESI-MS m/z calc. 684.3094, found 685.59 (M+1)⁺; Retention time: 1.35 minutes, LC method A.

### Example 22: Preparation of (Compound 50)

### Step 1: 3-[[4-[(2R)-2-Amino-3-[1-(trifluoromethyl)cyclopropyl]propoxy]-6-(2,6-dimethylphenyl)-5-methyl-pyrimidin-2-yl]sulfamoyl]benzoic acid

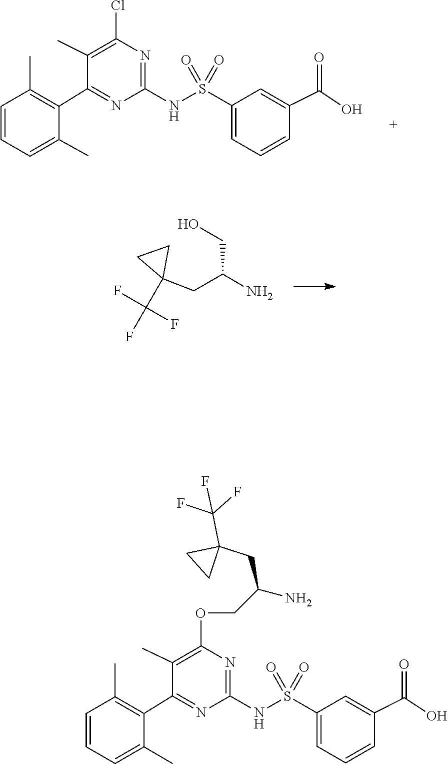 [SMILES: Cc1cccc(C)c1-c1nc(NS(=O)(=O)c2cccc(C(=O)O)c2)nc(Cl)c1C ; N[C@@H](CO)CC1(C(F)(F)F)CC1 ; Cc1cccc(C)c1-c1nc(NS(=O)(=O)c2cccc(C(=O)O)c2)nc(OC[C@H](N)CC2(C(F)(F)F)CC2)c1C]

3-[[4-Chloro-6-(2,6-dimethylphenyl)-5-methyl-pyrimidin-2-yl]sulfamoyl]benzoic acid (1.058 g, 2.450 mmol) and (2R)-2-amino-3-[1-(trifluoromethyl)cyclopropyl]propan-1-ol (hydrochloride salt) (544.6 mg, 2.480 mmol) were combined in THF (6 mL) and sodium tert-butoxide (1.085 g, 11.29 mmol) was added. The reaction mixture was stirred at room temperature for 30 min. The mixture was partitioned between ethyl acetate and a 1M HCl solution. The organics were separated, washed with brine, dried over sodium sulfate and evaporated to give 3-[[4-[(2R)-2-amino-3-[1-(trifluoromethyl)cyclopropyl]propoxy]-6-(2,6-dimethylphenyl)-5-methyl-pyrimidin-2-yl]sulfamoyl]benzoic acid (hydrochloride salt) (1.4547 g, 91%) ESI-MS m/z calc. 578.1811, found 579.3 (M+1)⁺; Retention time: 0.46 minutes, LC method D.

### Step 2: 3-[[4-(2,6-Dimethylphenyl)-5-methyl-6-[(2R)-2-(spiro[2.3]hexan-5-ylamino)-3-[1-(trifluoromethyl)cyclopropyl]propoxy]pyrimidin-2-yl]sulfamoyl]benzoic acid

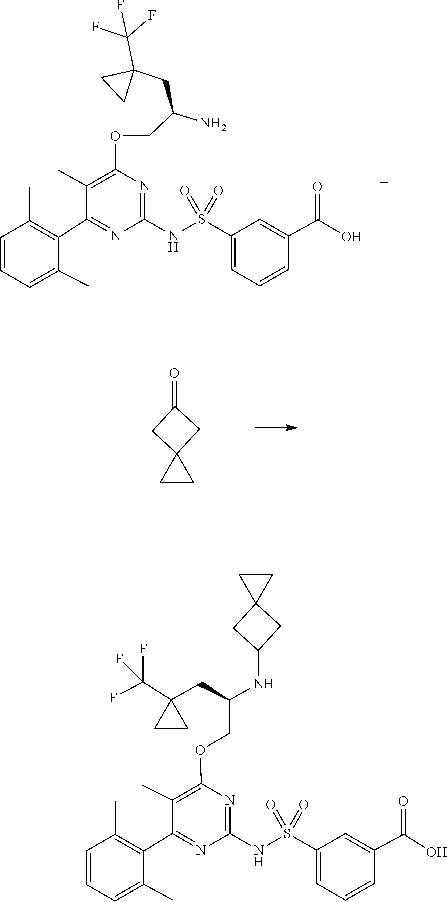 [SMILES: Cc1cccc(C)c1-c1nc(NS(=O)(=O)c2cccc(C(=O)O)c2)nc(OC[C@H](N)CC2(C(F)(F)F)CC2)c1C ; O=C1CC2(CC2)C1 ; Cc1cccc(C)c1-c1nc(NS(=O)(=O)c2cccc(C(=O)O)c2)nc(OC[C@@H](CC2(C(F)(F)F)CC2)NC2CC3(CC3)C2)c1C]

A 4 mL vial was charged with 3-[[4-[(2R)-2-amino-3-[1-(trifluoromethyl)cyclopropyl]propoxy]-6-(2,6-dimethylphenyl)-5-methyl-pyrimidin-2-yl]sulfamoyl]benzoic acid (hydrochloride salt) (67 mg, 0.1089 mmol), spiro[2.3]hexan-5-one (26 mg, 0.2705 mmol), anhydrous DCM (200 μL) and sodium triacetoxyborohydride (Sodium salt) (78 mg, 0.3680 mmol). The vial was briefly purged with nitrogen and the mixture was stirred at rt for 2 hours. A bit of methanol and water were added (100 μL each) and the mixture was concentrated and dissolved in DMSO (total final volume 2 mL). The mixture was purified by reverse phase preparative HPLC (Cis) using a gradient of acetonitrile in water (1 to 99% over 15 min) and HCl as a modifier. The pure fractions were collected and the solvents evaporated to give 3-[[4-(2,6-dimethylphenyl)-5-methyl-6-[(2R)-2-(spiro[2.3]hexan-5-ylamino)-3-[1-(trifluoromethyl)cyclopropyl]propoxy]pyrimidin-2-yl]sulfamoyl]benzoic acid (hydrochloride salt) (50 mg, 66%) as a white solid. ESI-MS m/z calc. 658.24365, found 659.37 (M+1)⁺; Retention time: 1.59 minutes, LC method A.

### Step 3: (11R)-6-(2,6-Dimethylphenyl)-7-methyl-2,2-dioxo-12-spiro[2.3]hexan-5-yl-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (Compound 50)

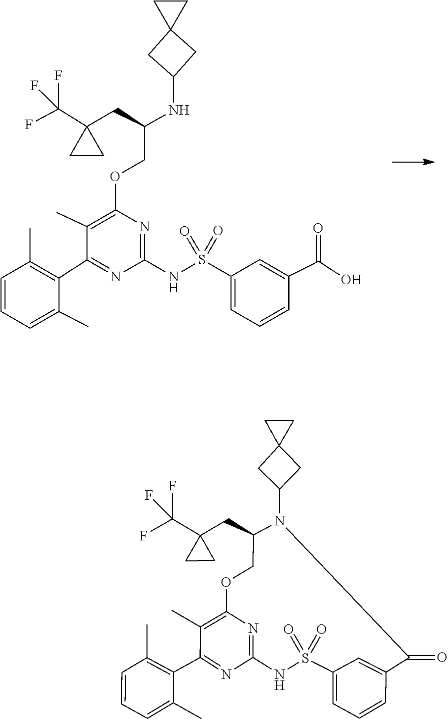 [SMILES: Cc1cccc(C)c1-c1nc(NS(=O)(=O)c2cccc(C(=O)O)c2)nc(OC[C@@H](CC2(C(F)(F)F)CC2)NC2CC3(CC3)C2)c1C ; Cc1cccc(C)c1-c1nc2nc(c1C)OC[C@@H](CC1(C(F)(F)F)CC1)N(C1CC3(CC3)C1)C(=O)c1cccc(c1)S(=O)(=O)N2]

A 20 mL flask was charged under nitrogen with HATU (57 mg, 0.1499 mmol), anhydrous DMF (3 mL) and DIEA (70 μL, 0.4019 mmol). A solution of 3-[[4-(2,6-dimethylphenyl)-5-methyl-6-[(2R)-2-(spiro[2.3]hexan-5-ylamino)-3-[1-(trifluoromethyl)cyclopropyl]propoxy]pyrimidin-2-yl]sulfamoyl]benzoic acid (hydrochloride salt) (50 mg, 0.07192 mmol) in anhydrous DMF (2 mL) was added dropwise through syringe over a period of 2 minutes. The mixture was stirred at room temperature for 23 hours. The mixture was concentrated and diluted with DMSO (2 mL). The solution was microfiltered through a syringe filter disc and purified by reverse phase preparative HPLC (Cis) using a gradient of acetonitrile in water (1 to 99% over 15 min) and HCl as a modifier to give (11R)-6-(2,6-dimethylphenyl)-7-methyl-2,2-dioxo-12-spiro[2.3]hexan-5-yl-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (20.6 mg, 44%) as a white solid. ESI-MS m/z calc. 640.2331, found 641.57 (M+1)⁺; Retention time: 2.05 minutes, LC method A.

### Example 23: Preparation of Compound 51

### Step 1: tert-Butyl N-[2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate, diastereomer 1, and tert-butyl N-[2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate, diastereomer 2

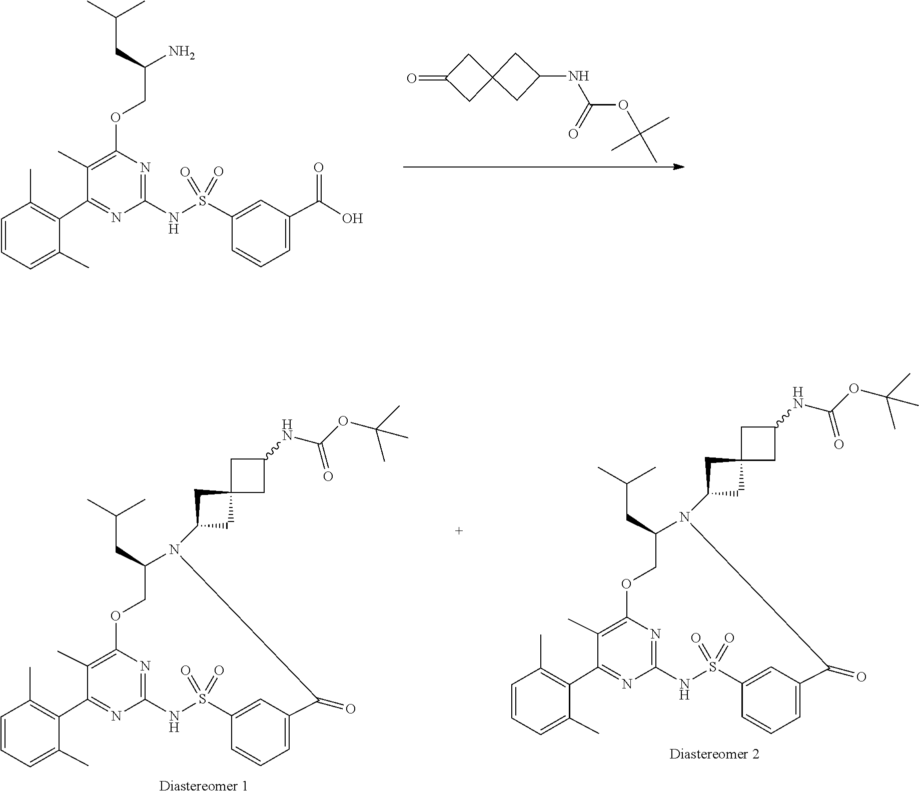

3-[[4-[(2R)-2-amino-4-methyl-pentoxy]-6-(2,6-dimethylphenyl)-5-methyl-pyrimidin-2-yl]sulfamoyl]benzoic acid (hydrochloride salt) (310 mg, 0.5646 mmol) was combined with tert-butyl N-(2-oxospiro[3.3]heptan-6-yl)carbamate (200 mg, 0.8878 mmol) in dichloromethane (0.6 mL), and stirred at room temperature for 5 minutes. sodium triacetoxyborohydride (360 mg, 1.699 mmol) was added and the reaction mixture was stirred at room temperature for 90 minutes. An additional portion of sodium triacetoxyborohydride (360 mg, 1.699 mmol) was then added and the reaction mixture was stirred for an additional 90 minutes at room temperature, then was partitioned between 0.5M HCl and water and the layers were separated. The aqueous layer was extracted an additional 4× with ethyl acetate and the combined organics were washed with brine dried over sodium sulfate and concentrated to give a white solid, which was used in the next Step without further purification. The product was added as a solution in DMF (10 mL) to a stirring solution of HATU (325 mg, 0.8547 mmol) and DIPEA (500 μL, 2.871 mmol) in DMF (30 mL) over 5 minutes. The reaction mixture was stirred at room temperature for three hours the was partitioned between 0.5 M HCl and ethyl acetate. The organics were separated and the aqueous was extracted three additional times with ethyl acetate. The combined organics were washed with brine, dried over sodium sulfate, and concentrated. The resulting crude material was purified by silica get chromatograph using a gradient of 0-100% ethyl acetate in hexanes. Fractions containing product were combined to give tert-butyl N-[2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]spiro[3.3]heptan-6-yl]carbamate as a mixture of diastereomers (180 mg, 45%) ESI-MS m/z calc. 703.34033, found 704.5 (M+1)⁺; Retention time: 0.82 minutes (LC method D).

The product was subjected to SFC purification (Phenomenex LUX-4 (250×21.2 mm), 5 m; 40° C. 32% MeOH (no modifier), 68% CO₂, 70.0 mL/min, −30 mg/mL methanol (no modifier), 70 μL injection volume, 150 bar, 210 nm) to give separately diastereomer 1, tert-butyl N-[2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (64 mg, 16%) ESI-MS m/z calc. 703.34033, found 704.3 (M+1)⁺; Retention time: 0.87 minutes (peak 1) (LC method D); and diastereomer 2, tert-butyl N-[2-[(11R)-6-(2,6-dimethylphenyl)-11-isobutyl-7-methyl-2,2,13-trioxo-9-oxa-2⁶-thia-3,5,12,19-tetrazatricyclo[12.3.1.14,8]nonadeca-1(18),4,6,8(19),14,16-hexaen-12-yl]spiro[3.3]heptan-6-yl]carbamate (61 mg, 15%) ESI-MS m/z calc. 703.34033, found 704.3 (M+1)⁺; Retention time: 0.86 minutes (peak 2) (LC method D).

### Step 2: (11R)-12-(6-Aminospiro[3.3]heptan-2-yl)-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 2

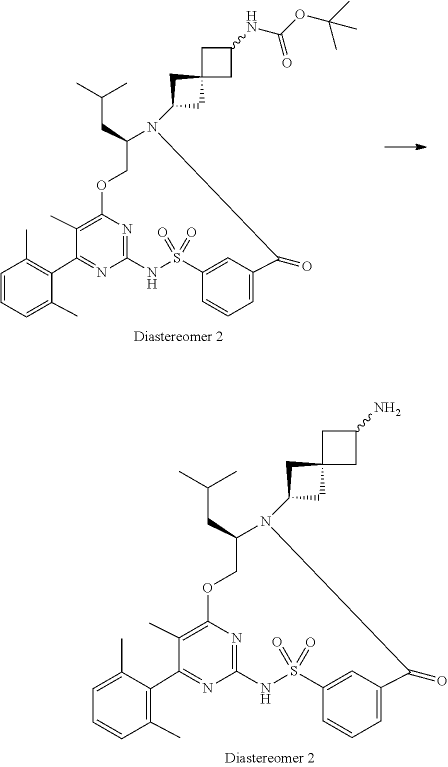

tert-Butyl N-[2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate, diastereomer 2 (61 mg, 0.0867 mmol) was combined in DCM (0.4 mL) with HCl (approximately 325.0 μL of 4 M, 1.30 mmol) and stirred for 30 minutes at room temperature. The reaction mixture was then concentrated, diluted with hexanes and concentrated a second time to give as a white solid, (11R)-12-(6-aminospiro[3.3]heptan-2-yl)-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one (hydrochloride salt), diastereomer 2 (54 mg, 97%). ESI-MS m/z calc. 603.2879, found 604.3 (M+1)⁺; Retention time: 0.58 minutes; LC method D.

### Step 3: Isopropyl N-[2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (Compound 51)

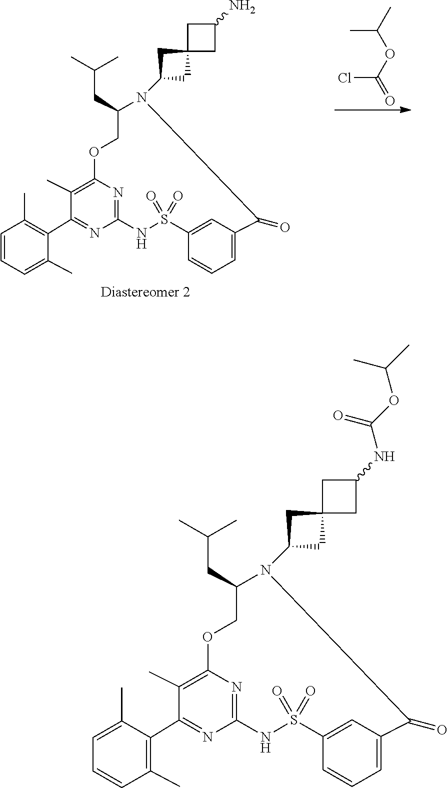

(11R)-12-(6-Aminospiro[3.3]heptan-2-yl)-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one (hydrochloride salt), diastereomer 2 (12 mg, 0.01874 mmol) was combined with DIPEA (approximately 12.11 mg, 16.32 μL, 0.09370 mmol) and isopropyl chloroformate (approximately 18.74 μL of 2 M, 0.03748 mmol) in DCM (0.5 mL) and stirred at room temperature for 10 minutes. The reaction mixture was then quenched with several drops of 1 M HCl, and partially concentrated. The crude material was dissolved in 1:1 DMSO/methanol, filtered and purified by prep HPLC (1-99% ACN in water, HCl modifier, 15 minute run) to give isopropyl N-[2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (6.8 mg, 52%). ESI-MS m/z calc. 689.3247, found 690.7 (M+1)⁺; Retention time: 2.02 minutes; LC method A.

### Example 24: Preparation of Compound 52

### Step 1: (11R)-12-(6-Aminospiro[3.3]heptan-2-yl)-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 1

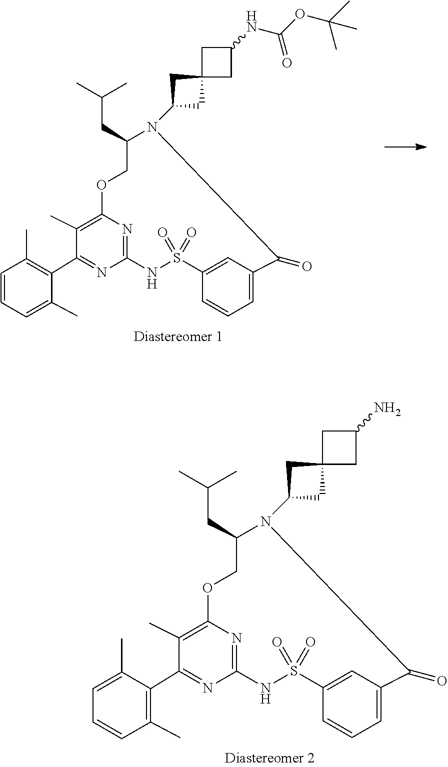

tert-Butyl N-[2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate, diastereomer 1 (64 mg, 0.09092 mmol) was combined in DCM (0.4 mL) with HCl (approximately 341.0 μL of 4 M, 1.364 mmol) and stirred for 30 minutes at room temperature. The reaction mixture was then concentrated, diluted with hexanes and concentrated a second time to give as a white solid, (11R)-12-(6-aminospiro[3.3]heptan-2-yl)-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 1 (hydrochloride salt) (58 mg, 100%). ESI-MS m/z calc. 603.2879, found 604.2 (M+1)⁺; Retention time: 0.57 minutes; LC method D.

### Step 2: Isopropyl N-[2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (Compound 52)

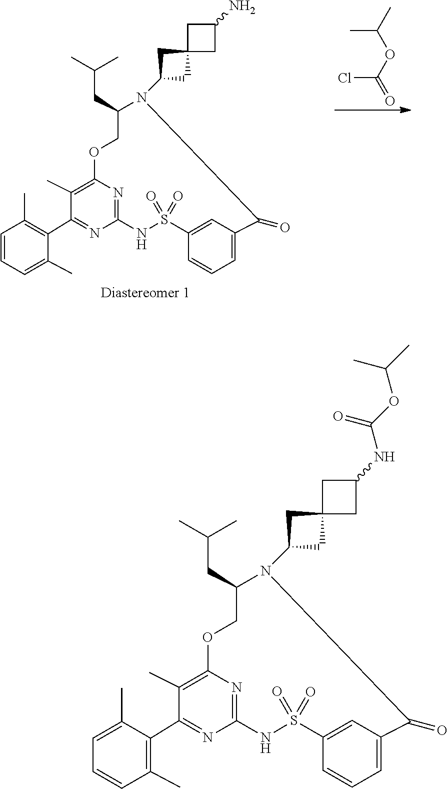

(11R)-12-(6-Aminospiro[3.3]heptan-2-yl)-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 1 (hydrochloride salt) (12 mg, 0.01874 mmol) was combined with DIPEA (approximately 12.11 mg, 16.32 μL, 0.09370 mmol) and isopropyl chloroformate (approximately 18.74 μL of 2 M, 0.03748 mmol) in DCM (0.5 mL) and stirred at room temperature for 10 minutes. The reaction mixture was then quenched with several drops of 1M HCl, and partially concentrated. The crude material was dissolved in 1:1 DMSO/methanol, filtered and purified by prep HPLC (1-99% ACN in water, HCl modifier, 15 minute run) to give isopropyl N-[2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (7.6 mg, 58%). ESI-MS m/z calc. 689.3247, found 690.7 (M+1)⁺; Retention time: 2.03 minutes; LC method A.

### Example 25: Preparation of Compound 53

### Step 1: (11R)-12-[2-(Dimethylamino)spiro[3.3]heptan-6-yl]-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one (Compound 53)

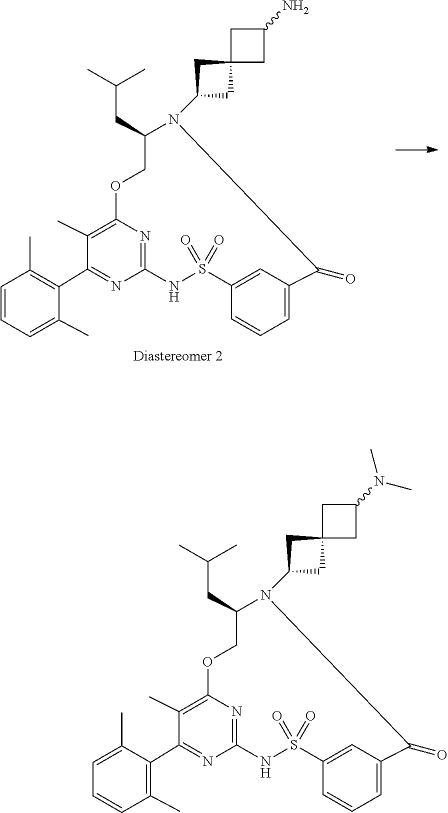

(11R)-12-(6-Aminospiro[3.3]heptan-2-yl)-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 2 (hydrochloride salt) (18 mg, 0.02811 mmol) was combined with formic acid (0.2 mL) and aqueous formaldehyde (approximately 342.1 mg, 313.9 μL, 4.216 mmol) in a screwcap vial with an unpierced septum and heated to 95° C. for 16 hours. The reaction mixture was then cooled to room temperature, diluted with methanol, filtered and purified by prep HPLC (1-70% ACN in water HCl modifier, 15 minute run) to give (11R)-12-[2-(dimethylamino)spiro[3.3]heptan-6-yl]-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one (hydrochloride salt) (6.9 mg, 36%). ESI-MS m/z calc. 631.3192, found 632.7 (M+1)⁺; Retention time: 1.34 minutes; LC method A.

### Example 26: Preparation of Compound 54

### Step 1: (11R)-12-[2-(Dimethylamino)spiro[3.3]heptan-6-yl]-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one (Compound 54)

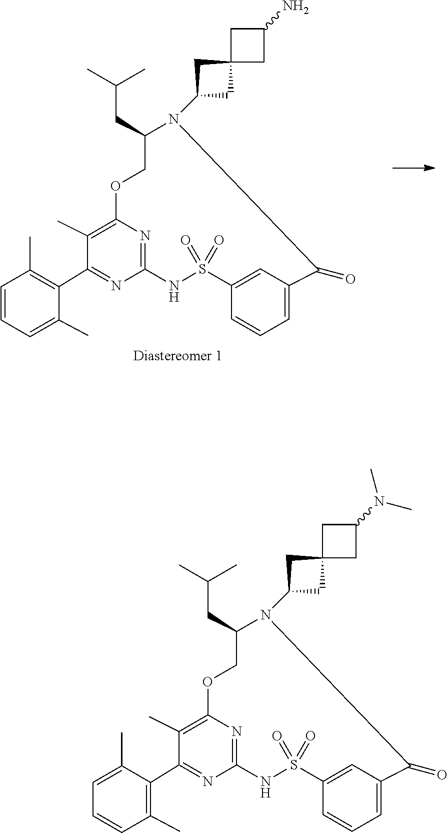

(11R)-12-(6-Aminospiro[3.3]heptan-2-yl)-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 1 (hydrochloride salt) (18 mg, 0.02811 mmol) was combined with formic acid (0.2 mL) and aqueous formaldehyde (approximately 342.1 mg, 313.9 μL, 4.216 mmol) in a screwcap vial with an unpierced septum and heated to 95° C. for 16 hours. The reaction mixture was then cooled to room temperature, diluted with methanol, filtered and purified by prep HPLC (1-70% ACN in water HCl modifier, 15 minute run) to give (11R)-12-[2-(dimethylamino)spiro[3.3]heptan-6-yl]-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one (hydrochloride salt) (7.7 mg, 41%). ESI-MS m/z calc. 631.3192, found 632.9 (M+1)⁺; Retention time: 1.36 minutes; LC method A.

### Example 27: Preparation of Compound 55

### Step 1: tert-Butyl 2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate

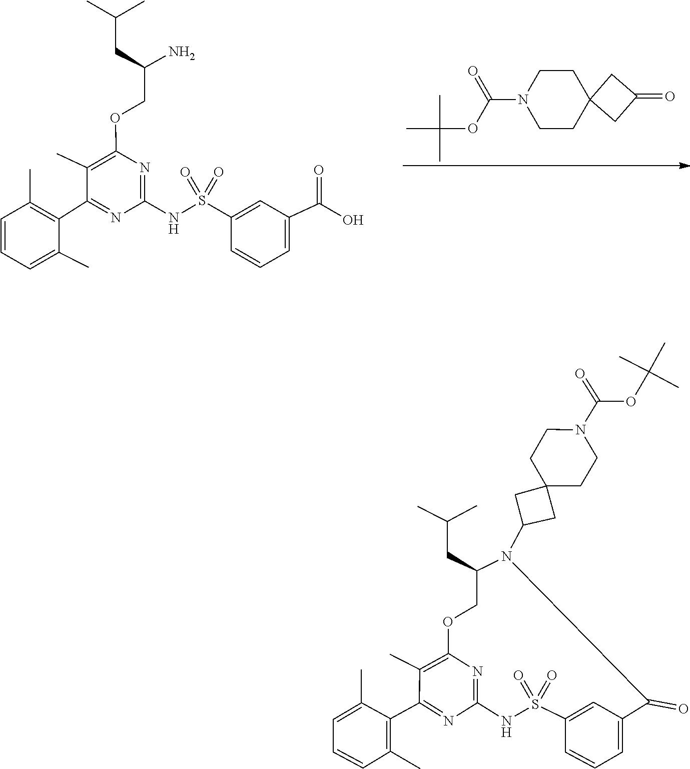 [SMILES: Cc1cccc(C)c1-c1nc(NS(=O)(=O)c2cccc(C(=O)O)c2)nc(OC[C@H](N)CC(C)C)c1C ; CC(C)(C)OC(=O)N1CCC2(CC1)CC(=O)C2 ; Cc1cccc(C)c1-c1nc2nc(c1C)OC[C@@H](CC(C)C)N(C1CC3(CCN(C(=O)OC(C)(C)C)CC3)C1)C(=O)c1cccc(c1)S(=O)(=O)N2]

3-[[4-[(2R)-2-Amino-4-methyl-pentoxy]-6-(2,6-dimethylphenyl)-5-methyl-pyrimidin-2-yl]sulfamoyl]benzoic acid (hydrochloride salt) (100 mg, 0.1821 mmol) was combined with the tert-butyl 2-oxo-7-azaspiro[3.5]nonane-7-carboxylate (approximately 65.38 mg, 0.2732 mmol) derivative in DCM (0.3 mL) at room temperature. The reaction mixture was stirred for 5 minutes and sodium triacetoxyborohydride (approximately 115.8 mg, 0.5463 mmol) was added. After one hour an additional portion of sodium triacetoxyborohydride (approximately 57.90 mg, 0.2732 mmol) added, followed by a further portion of sodium triacetoxyborohydride (approximately 57.90 mg, 0.2732 mmol) 45 minutes later. After 2.5 hours total reaction time the reaction mixture was partitioned between 0.5M HCl and ethyl acetate and the layers were separated. The aqueous layer was extracted an additional three times with ethyl acetate, and the combined organics were washed with brine, dried over sodium sulfate and concentrated. The resulting crude white solids were used in the second Step without further purification. The product was dissolved in DMF (10 mL) and added to a stirring solution of HATU (approximately 90.00 mg, 0.2367 mmol) and DIPEA (approximately 117.7 mg, 158.6 μL, 0.9105 mmol) in DMF (10 mL), and stirred at room temperature for three hours. The reaction mixture was then partitioned between 1M HCl and ethyl acetate. The layers were separated, and the aqueous layer was extracted an additional three times with ethyl acetate. The combined organics were washed with brine and dried over sodium sulfate then concentrated. The resulting crude material was purified by chromatography on silica gel eluting with 0-100% ethyl acetate in hexanes to give tert-butyl 2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (60 mg, 46%).

### Step 2: (11R)-12-(7-Azaspiro[3.5]nonan-2-yl)-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one

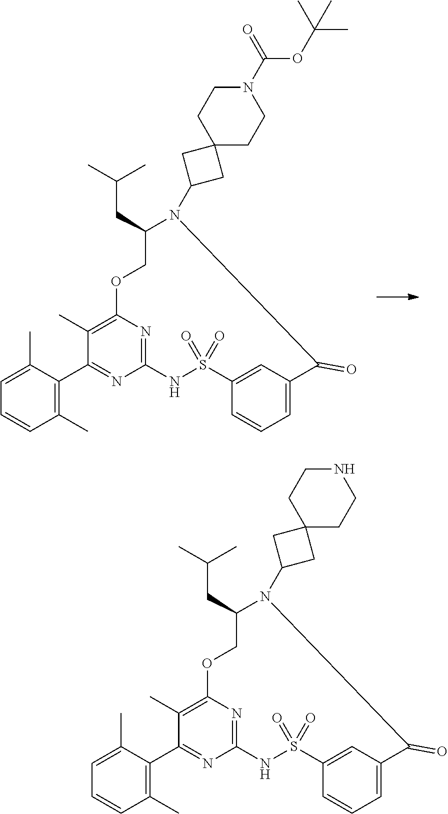 [SMILES: Cc1cccc(C)c1-c1nc2nc(c1C)OC[C@@H](CC(C)C)N(C1CC3(CCN(C(=O)OC(C)(C)C)CC3)C1)C(=O)c1cccc(c1)S(=O)(=O)N2 ; Cc1cccc(C)c1-c1nc2nc(c1C)OC[C@@H](CC(C)C)N(C1CC3(CCNCC3)C1)C(=O)c1cccc(c1)S(=O)(=O)N2]

tert-Butyl 2-[(11R)-6-(2,6-dimethylphenyl)-11-isobutyl-7-methyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (60 mg, 0.08358 mmol) was combined in DCM (0.3 mL) with HCl (350 μL of 4 M, 1.400 mmol) in dioxane and stirred at room temperature for 20 minutes. The reaction mixture was then evaporated. Hexanes were added and the reaction mixture was evaporated a second time to give a yellowish solid, which was used in the next step without further purification, (11R)-12-(7-azaspiro[3.5]nonan-2-yl)-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (54 mg, 99%) ESI-MS m/z calc. 617.3036, found 618.7 (M+1)⁺; Retention time: 1.3 minutes; LC method A.

### Step 3: (11R)-6-(2,6-Dimethylphenyl)-11-isobutyl-12-[7-(2-methoxyethyl)-7-azaspiro[3.5]nonan-2-yl]-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 55)

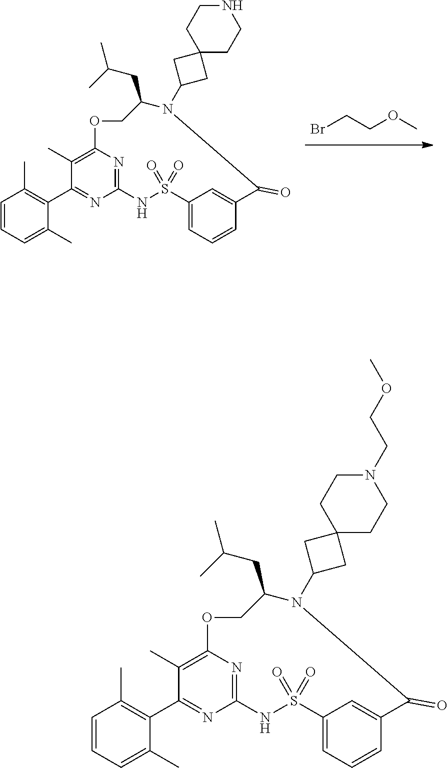

(11R)-12-(7-Azaspiro[3.5]nonan-2-yl)-6-(2,6-dimethylphenyl)-11-isobutyl-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (20 mg, 0.03057 mmol) was combined with triethylamine (30 μL, 0.2152 mmol) in acetonitrile (300 μL) in a screwcap vial, and 1-bromo-2-methoxy-ethane (4 μL, 0.04256 mmol) was added. The reaction mixture was heated to 55° C. for 22 hours. The reaction mixture was cooled to room temperature, diluted with methanol, filtered, and purified by preparative HPLC(1-99% ACN in water, 30 minute run, HCl modifier) to give (11R)-6-(2,6-dimethylphenyl)-11-isobutyl-12-[7-(2-methoxyethyl)-7-azaspiro[3.5]nonan-2-yl]-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (4.1 mg, 19%). ESI-MS m/z calc. 675.34546, found 676.6 (M+1)⁺; Retention time: 1.36 minutes; LC method A.

### Example 28: Preparation of Compound 56

### Step 1: (2R)-4,4-dimethyl-2-[(1-methylpyrazol-4-yl)amino]pentan-1-ol

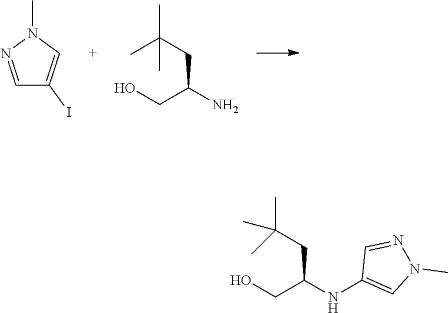

(2R)-2-Amino-4,4-dimethyl-pentan-1-ol (hydrochloride salt) (5 g, 29.819 mmol), 4-iodo-1-methyl-pyrazole (6.2 g, 29.808 mmol), CuI (568 mg, 2.9824 mmol) and NaOH (6 g, 150.01 mmol) were placed in a 100 mL flask. The flask was evacuated and purged with nitrogen three times. DMSO (30 mL) and water (30 mL) were added and the resulting mixture was stirred at 90° C. for 20 h under an atmosphere of nitrogen. The solution was cooled down to room temperature and was filtered on a Celite pad, rinsed with MeOH (300 mL). The filtrate was quenched with aqueous saturated ammonium chloride (300 mL) and the volatiles were removed under reduced pressure. The product was extracted with DCM (4×200 mL). Combined organic layers were washed with water (3×100 mL), a 1:1 mix of water and brine (2×100 mL), brine (2×100 mL), dried over sodium sulfate, filtered and concentrated under reduced pressure. The crude was purified using flash-chromatography on a 330 g silica gel cartridge, using a gradient of MeOH in DCM of 2 to 5% in 11 CV. Removal of the volatiles under reduced pressure afforded a purple solid which was purified by reverse phase chromatography on a 175 g Cis cartridge using a gradient of MeOH in acidic water (0.1% v/v of formic acid in water) of 40 to 100% for 11 CV then 100% for 3 CV to give two lots of the product. The fractions containing the first lot were evaporated and then lyophilized. Afforded (2R)-4,4-dimethyl-2-[(1-methylpyrazol-4-yl)amino]pentan-1-ol (1.1 g, 17%) as a white solid. ¹H NMR (400 MHz, CDCl₃) δ 7.13 (s, 1H), 6.96 (s, 1H), 3.81 (s, 3H), 3.68 (dd, J=10.8, 4.4 Hz, 1H), 3.36 (dd, J=10.8, 6.6 Hz, 1H), 3.11-3.03 (m, 1H), 2.65-2.35 (br. s, 2H), 1.39-1.36 (m, 2H), 0.94 (s, 9H). ESI-MS m/z calc. 211.1685, found 212.4 (M+1)⁺; Retention time: 1.12 minutes (LC method X). The fractions containing the second lot were evaporated and then lyophilized. Afforded (2R)-4,4-dimethyl-2-[(1-methylpyrazol-4-yl)amino]pentan-1-ol (1.2 g, 19%) as a white solid. ESI-MS m/z calc. 211.1685, found 212.4 (M+1)⁺; Retention time: 1.12 minutes (LC method X).

### Step 2: 3-[[4-[(2R)-4,4-Dimethyl-2-[(1-methylpyrazol-4-yl)amino]pentoxy]-6-(2,6-dimethylphenyl)-5-methyl-pyrimidin-2-yl]sulfamoyl]benzoic acid

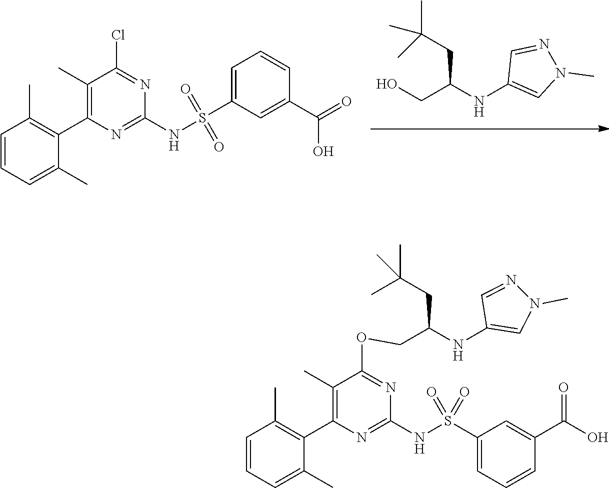 [SMILES: Cc1cccc(C)c1-c1nc(NS(=O)(=O)c2cccc(C(=O)O)c2)nc(Cl)c1C ; Cn1cc(N[C@@H](CO)CC(C)(C)C)cn1 ; Cc1cccc(C)c1-c1nc(NS(=O)(=O)c2cccc(C(=O)O)c2)nc(OC[C@@H](CC(C)(C)C)Nc2cnn(C)c2)c1C]

To a 0° C. solution of 3-[[4-chloro-6-(2,6-dimethylphenyl)-5-methyl-pyrimidin-2-yl]sulfamoyl]benzoic acid (43 mg, 0.0950 mmol) and (2R)-4,4-dimethyl-2-[(1-methylpyrazol-4-yl)amino]pentan-1-ol (20 mg, 0.0947 mmol) in THF (1 mL) was added sodium tert-butoxide (46 mg, 0.4787 mmol) and the mixture was stirred for 1 h at rt. 1 N aqueous HCl (3 mL) was added at 0° C. and the product was extracted with ethyl acetate (3×10 mL). The combined organic layers were washed with brine (10 mL), dried over sodium sulfate, filtered and concentrated under reduced pressure. Afforded 3-[[4-[(2R)-4,4-dimethyl-2-[(1-methylpyrazol-4-yl)amino]pentoxy]-6-(2,6-dimethylphenyl)-5-methyl-pyrimidin-2-yl]sulfamoyl]benzoic acid (hydrochloride salt) (56 mg, 82%) as red semi-solid. ESI-MS m/z calc. 606.26245, found 607.4 (M+1)⁺; Retention time: 1.72 minutes; LC method X.

### Step 3: (11R)-6-(2,6-Dimethylphenyl)-11-(2,2-dimethylpropyl)-7-methyl-12-(1-methylpyrazol-4-yl)-2,2-dioxo-9-oxa-2λ⁶-thia-3,5,12,19-tetrazatricyclo[12.3.1.14,8]nonadeca-1(18),4(19),5,7,14,16-hexaen-13-one

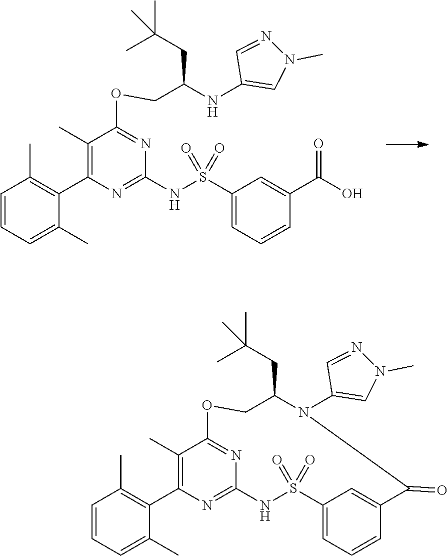 [SMILES: Cc1cccc(C)c1-c1nc(NS(=O)(=O)c2cccc(C(=O)O)c2)nc(OC[C@@H](CC(C)(C)C)Nc2cnn(C)c2)c1C ; Cc1cccc(C)c1-c1nc2nc(c1C)OC[C@@H](CC(C)(C)C)N(c1cnn(C)c1)C(=O)c1cccc(c1)S(=O)(=O)N2]

To a 0° C. solution of N-methylmorpholine (368.00 mg, 0.4 mL, 3.6383 mmol) in DMF (50 mL) was added 2-chloro-4,6-dimethoxy-1,3,5-triazine (110 mg, 0.6265 mmol) followed by 3-[[4-[(2R)-4,4-dimethyl-2-[(1-methylpyrazol-4-yl)amino]pentoxy]-6-(2,6-dimethylphenyl)-5-methyl-pyrimidin-2-yl]sulfamoyl]benzoic acid (303 mg, 0.3780 mmol). After 5 min the reaction was stirred at room temperature for 6 days. The reaction mixture was concentrated under reduced pressure at 50° C. The residue was purified by reverse phase chromatography on a 20 g Cis cartridge using a gradient of MeCN in acidic water (0.1% v/v of formic acid in water) of 40 to 100% for 15 CV then 100% for 2 CV. Fractions containing the product were evaporated and then lyophilized. Afforded (11R)-6-(2,6-dimethylphenyl)-11-(2,2-dimethylpropyl)-7-methyl-12-(1-methylpyrazol-4-yl)-2,2-dioxo-9-oxa-2λ⁶-thia-3,5,12,19-tetrazatricyclo[12.3.1.14,8]nonadeca-1(18),4(19),5,7,14,16-hexaen-13-one (73 mg, 32%) as a white solid. ESI-MS m/z calc. 588.2519, found 589.3 (M+1)⁺; Retention time: 3.91 minutes; LC method Y.

### Step 4: (11R)-12-(5-Bromo-1-methyl-pyrazol-4-yl)-6-(2,6-dimethylphenyl)-11-(2,2-dimethylpropyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 56)

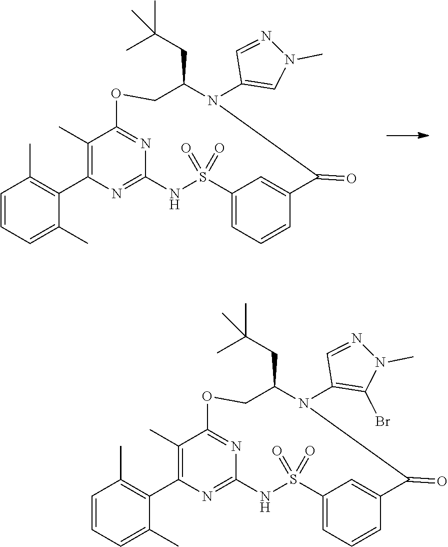 [SMILES: Cc1cccc(C)c1-c1nc2nc(c1C)OC[C@@H](CC(C)(C)C)N(c1cnn(C)c1)C(=O)c1cccc(c1)S(=O)(=O)N2 ; Cc1cccc(C)c1-c1nc2nc(c1C)OC[C@@H](CC(C)(C)C)N(c1cnn(C)c1Br)C(=O)c1cccc(c1)S(=O)(=O)N2]

In a reaction tube was introduced (11R)-6-(2,6-dimethylphenyl)-11-(2,2-dimethylpropyl)-7-methyl-12-(1-methylpyrazol-4-yl)-2,2-dioxo-9-oxa-2λ⁶-thia-3,5,12,19-tetrazatricyclo[12.3.1.14,8]nonadeca-1(18),4(19),5,7,14,16-hexaen-13-one (38 mg, 0.0627 mmol), DMF (0.5 mL) and NBS (17 mg, 0.0955 mmol) were added. The tube was then sealed and stir at 80° C. for 1 hour. The reaction mixture was concentrated under reduced pressure at 50° C. The crude product was purified by reverse phase chromatography on a 20 g Cis cartridge using a gradient of MeCN in acidic water (0.1% v/v of formic acid in water) of 40 to 100% for 15 CV then 100% for 2 CV to give two lots of the product. The fractions containing the first lot were evaporated and then lyophilized. Afforded (11R)-12-(5-bromo-1-methyl-pyrazol-4-yl)-6-(2,6-dimethylphenyl)-11-(2,2-dimethylpropyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (11 mg, 22%) as a white solid. ESI-MS m/z calc. 666.1624, found 667.2 (M+1)⁺; Retention time: 4.22 minutes (LC method Y). The fractions containing a second lot were evaporated and then lyophilized. Afforded (11R)-12-(5-bromo-1-methyl-pyrazol-4-yl)-6-(2,6-dimethylphenyl)-11-(2,2-dimethylpropyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (26 mg, 60%) as a white solid. ESI-MS m/z calc. 666.1624, found 667.2 (M+1)⁺; Retention time: 4.23 minutes (LC method Y).

### Example 29: Preparation of Compound 57 and Compound 58

### Step 1: Methyl 2-[[(1R)-1-(hydroxymethyl)-3,3-dimethyl-butyl]amino]spiro[3.3]heptane-6-carboxylate

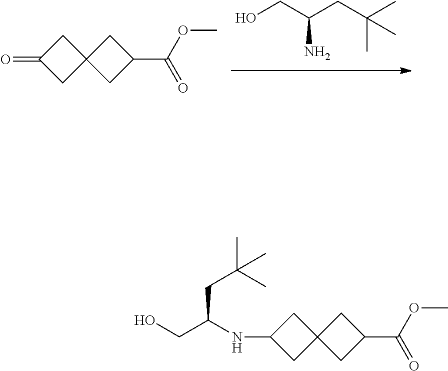

A 500 mL round bottom flask equipped with a magnetic stir bar was charged under nitrogen with methyl 2-oxospiro[3.3]heptane-6-carboxylate (12.18 g, 72.42 mmol) and anhydrous DCE (200 mL). Stirring was commenced and (2R)-2-amino-4,4-dimethyl-pentan-1-ol (hydrochloride salt) (12.2 g, 72.76 mmol) was added. To the suspension, DIEA (15 mL, 86.12 mmol) and acetic acid (4.8 mL, 84.41 mmol) were added and the reaction was stirred at rt for 5-10 min (complete dissolution). sodium triacetoxyborohydride (22.5 g, 106.2 mmol) was added and stirring was continued at rt for 16 h. The reaction was cooled down in an ice-bath (internal temperature 2° C.) and was quenched by slow addition of aqueous HCl (40 mL of 4 M, 160.0 mmol) while maintaining temperature below 7° C. A suspension of sodium bicarbonate (45 g, 535.7 mmol) in water (100 mL) was slowly added (foaming) while maintaining temperature below 10° C. More water (50 mL) and brine (50 mL) were added and the mixture was stirred until gas evolution stopped (final pH=7-8). The two phases were separated, and the aqueous phase was further extracted with DCM (3×50 mL). The combined extracts were washed with brine (50 ml), dried over sodium sulfate and filtered through a pad of Celite. Evaporation of the solvents gave methyl 2-[[(1R)-1-(hydroxymethyl)-3,3-dimethyl-butyl]amino]spiro[3.3]heptane-6-carboxylate (21.08 g, 100%) as a white solid. ESI-MS m/z calc. 283.21475, found 284.12 (M+1)⁺; Retention time: 0.88 minutes (LC method A). ¹H NMR (400 MHz, DMSO-d₆) δ 7.77 (broad s, 1H), 5.23 (broad s, 1H), 3.57 (s, 3H), 3.54-3.42 (m, 2H), 3.31 (dd, J=11.8, 5.9 Hz, 1H), 3.09-2.97 (m, 1H), 2.73 (br s, 1H), 2.40-1.95 (m, 8H), 1.39 (dd, J=14.4, 7.6 Hz, 1H), 1.25 (dd, J=14.4, 2.4 Hz, 1H), 0.89 (s, 9H).

### Step 2: (2R)-2-[[6-(1-Hydroxy-1-methyl-ethyl)spiro[3.3]heptan-2-yl]amino]-4,4-dimethyl-pentan-1-ol

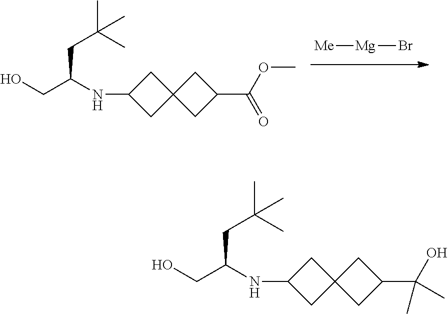

A 100 mL flask was charged under nitrogen with methyl 2-[[(1R)-1-(hydroxymethyl)-3,3-dimethyl-butyl]amino]spiro[3.3]heptane-6-carboxylate (322 mg, 1.136 mmol) and anhydrous THF (7 mL). The mixture was cooled down in an ice bath and MeMgBr (1.7 mL of 3 M, 5.100 mmol) (3M in diethyl ether) was added dropwise through syringe over a period of 5 minutes (gas evolution visible at the beginning of the addition). At the end of the addition (clear solution), the ice bath was removed, and the reaction was stirred at room temperature for 1 hour. More THF (4 mL) was added to ease stirring. After 30 min, more MeMgBr (0.2 mL of 3 M, 0.6000 mmol) was added and the mixture was stirred at room temperature for 6 h. The reaction was cooled down and treated with saturated ammonium chloride (50 ml), Brine (30 mL) and ethyl acetate (40 mL). The two phases were separated, and the aqueous phase was extracted with ethyl acetate (3×30 mL). The combined extracts were washed with brine (40 mL) dried over sodium sulfate and the solvents evaporated to give crude (2R)-2-[[6-(1-hydroxy-1-methyl-ethyl)spiro[3.3]heptan-2-yl]amino]-4,4-dimethyl-pentan-1-ol (288 mg, 89%) as a colorless resin. ESI-MS m/z calc. 283.25113, found 284.18 (M+1)⁺; Retention time: 0.91 minutes. second isomer visible: retention time: 0.93 minutes (ratio 1:1) (LC method A).

### Step 3: 3-[[4-(2,6-dimethylphenyl)-6-[(2R)-2-[[6-(1-hydroxy-1-methyl-ethyl)spiro[3.3]heptan-2-yl]amino]-4,4-dimethyl-pentoxy]-5-methyl-pyrimidin-2-yl]sulfamoyl]benzoic acid

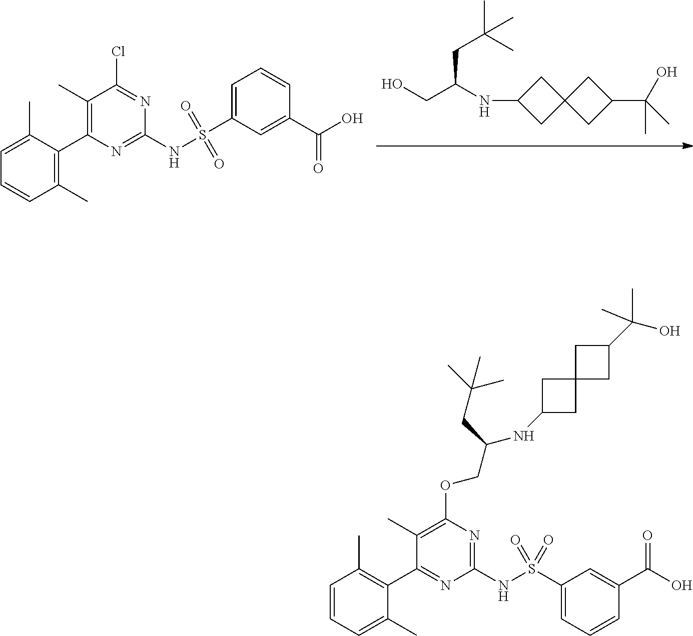 [SMILES: Cc1cccc(C)c1-c1nc(NS(=O)(=O)c2cccc(C(=O)O)c2)nc(Cl)c1C ; CC(C)(C)C[C@H](CO)NC1CC2(C1)CC(C(C)(C)O)C2 ; Cc1cccc(C)c1-c1nc(NS(=O)(=O)c2cccc(C(=O)O)c2)nc(OC[C@@H](CC(C)(C)C)NC2CC3(C2)CC(C(C)(C)O)C3)c1C]

In a 100 mL round bottom flask equipped with a magnetic stir bar and a nitrogen line was charged with 3-[[4-chloro-6-(2,6-dimethylphenyl)-5-methyl-pyrimidin-2-yl]sulfamoyl]benzoic acid (1.168 g, 2.704 mmol), (2R)-2-[[6-(1-hydroxy-1-methyl-ethyl)spiro[3.3]heptan-2-yl]amino]-4,4-dimethyl-pentan-1-ol (760 mg, 2.681 mmol), and anhydrous THF (10 mL) (clear solution). sodium tert-butoxide (1.35 g, 14.05 mmol) was added (slight exotherm observed). The mixture was stirred at room temperature for 2.5 h and then was stored in a freezer at −20° C. overnight. The reaction was warmed to room temperature and partitioned between ethyl acetate (50 mL), a 1M HCl solution (50 mL) and brine (50 mL). The aqueous phase was further extracted with ethyl acetate (2×30 mL). The combined organics were washed with brine (50 mL) dried over sodium sulfate, filtered over a pad of Celite and evaporated to dryness. The residue was triturated in ethyl acetate/diethyl ether (1:3 mixture, approximatively 150 mL). The suspension was stirred at room temperature for 2 h. The solid was filtered and dried to give crude 3-[[4-(2,6-dimethylphenyl)-6-[(2R)-2-[[6-(1-hydroxy-1-methyl-ethyl)spiro[3.3]heptan-2-yl]amino]-4,4-dimethyl-pentoxy]-5-methyl-pyrimidin-2-yl]sulfamoyl]benzoic acid (hydrochloride salt) (1.028 g, 54%) as an off-white solid. The crude solid was used for the next Step without any further purification. ESI-MS m/z calc. 678.3451, found 679.4 (M+1)⁺; Retention time: 1.34 minutes, second isomer: found 679.34 (M+1)⁺; retention time 1.35 minutes (isomer ratio 1:1) (LC method A).

### Step 4: (11R)-6-(2,6-Dimethylphenyl)-11-(2,2-dimethylpropyl)-12-[6-(2-hydroxypropan-2-yl)spiro[3.3]heptan-2-yl]-7-methyl-9-oxa-2λ⁶-thia-3,5,12,19-tetraazatricyclo[12.3.1.14,8]nonadeca-1(17),4(19),5,7,14(18),15-hexaene-2,2,13-trione, diastereomer 1 (Compound 57) and (11R)-6-(2,6-dimethylphenyl)-11-(2,2-dimethylpropyl)-12-[6-(2-hydroxypropan-2-yl)spiro[3.3]heptan-2-yl]-7-methyl-9-oxa-2λ⁶-thia-3,5,12,19-tetraazatricyclo[12.3.1.14,8]nonadeca-1(17),4(19),5,7,14(18),15-hexaene-2,2,13-trione, diastereomer 2 (Compound 58)

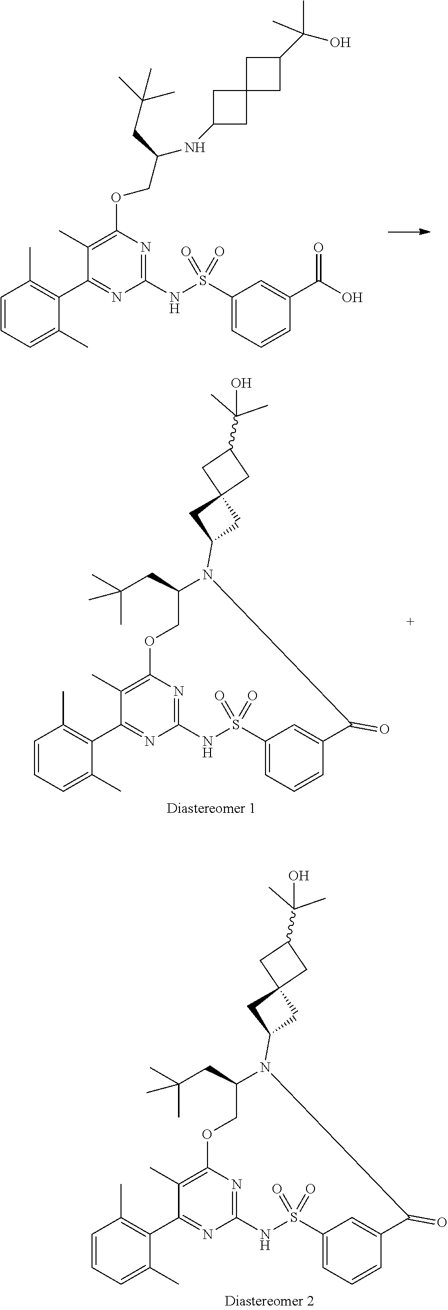

A 100 mL round bottom flask was charged under nitrogen with [dimethylamino(triazolo[4,5-b]pyridin-3-yloxy)methylene]-dimethyl-ammonium (Phosphorus Hexafluoride Ion) (1.159 g, 3.048 mmol) (HATU), anhydrous DMF (25 mL) and DIEA (1.3 mL, 7.463 mmol). A solution of crude 3-[[4-(2,6-dimethylphenyl)-6-[(2R)-2-[[6-(1-hydroxy-1-methyl-ethyl)spiro[3.3]heptan-2-yl]amino]-4,4-dimethyl-pentoxy]-5-methyl-pyrimidin-2-yl]sulfamoyl]benzoic acid (hydrochloride salt) (1.028 g, 1.437 mmol) in anhydrous DMF (25 mL) was added dropwise over a period of 5 minutes. The mixture was stirred at room temperature for 16 h. The solvent was evaporated, and the residue was partitioned between aqueous 1N HCl (50 mL) and ethyl acetate (30 mL). The two phases were separated, and the aqueous phase was further extracted with ethyl acetate (30 mL). The combined organic phases were dried over sodium sulfate and the solvents were evaporated. The product was dissolved in DCM/MeOH and purified by flash chromatography on silica gel (80 g column) using a gradient of ethyl acetate (0 to 100% over 30 min) in hexanes. The product eluted around 70-80% EA. Evaporation of the solvents gave a crude solid that was dissolved in DMSO (6 mL). The solution was microfiltered through syringe filter disc and purified by reverse phase preparative HPLC (Cis) using a gradient of acetonitrile in water (0-50% over 10 min, 50-70% over 20 min) and HCl as a modifier. Two isomers were baseline separated. Peak 1 eluted with an impurity. It was purified a second time using a 1-99% MeCN gradient to give a 92% pure material. It was purified a third time by flash chromatography on silica gel (4 g column) using a gradient of ethyl acetate (0 to 100% over 30 min) in hexanes. For each compound, the solvents were evaporated, and the residue was triturated in DCM/ethyl acetate/hexanes. Evaporation gave the two isomers as white solids: Diastereomer 1, more polar isomer, peak 1, (11R)-6-(2,6-dimethylphenyl)-11-(2,2-dimethylpropyl)-12-[6-(2-hydroxypropan-2-yl)spiro[3.3]heptan-2-yl]-7-methyl-9-oxa-2λ⁶-thia-3,5,12,19-tetraazatricyclo[12.3.1.14,8]nonadeca-1(17),4(19),5,7,14(18),15-hexaene-2,2,13-trione (29 mg, 6%). ESI-MS m/z calc. 660.33453, found 661.5 (M+1)⁺; Retention time: 1.96 minutes (LC method A), ¹H NMR (400 MHz, DMSO-d₆) δ 13.25-11.45 (broad m, 1H), 8.43 (s, 1H), 7.90 (s, 1H), 7.64 (s, 2H), 7.27 (s, 1H), 7.17 (d, J=7.6 Hz, 1H), 7.11 (s, 1H), 5.11 (br d, J=9.7 Hz, 1H), 4.52-4.17 (m, 1H), 3.98 (s, 1H), 3.88 (p, J=8.7 Hz, 1H), 3.67 (s, 1H), 3.03-2.83 (m, 2H), 2.35-2.23 (m, 1H), 2.20-1.90 (m, 8H), 1.83-1.54 (m, 8H), 1.43-1.30 (m, 1H), 0.97 (d, J=2.5 Hz, 6H), 0.46 (s, 9H); and diastereomer 2, less polar isomer, peak 2. (11R)-6-(2,6-dimethylphenyl)-11-(2,2-dimethylpropyl)-12-[6-(2-hydroxypropan-2-yl)spiro[3.3]heptan-2-yl]-7-methyl-9-oxa-2λ⁶-thia-3,5,12,19-tetraazatricyclo[12.3.1.14,8]nonadeca-1(17),4(19),5,7,14(18),15-hexaene-2,2,13-trione (60 mg, 13%). ESI-MS m/z calc. 660.33453, found 661.76 (M+1)⁺; Retention time: 2.01 minutes (LC method A), ¹H NMR (400 MHz, DMSO-d₆) δ 13.27-11.66 (broad m, 1H), 8.42 (s, 1H), 7.90 (s, 1H), 7.64 (s, 2H), 7.27 (s, 1H), 7.17 (m, 1H), 7.11 (m, 1H), 5.10 (s, 1H), 4.35 (br s, 1H), 3.99 (s, 1H), 3.88 (p, J=8.7 Hz, 1H), 3.75-3.59 (m, 1H), 3.01 (t, J=9.5 Hz, 1H), 2.85 (t, J=9.8 Hz, 1H), 2.40-2.27 (m, 1H), 2.19-1.89 (m, 8H), 1.88-1.48 (m, 8H), 1.36 (d, J=14.9 Hz, 1H), 0.97 (s, 6H), 0.47 (s, 9H).

### Example 30: Preparation of Compound 59, Compound 60, Compound 61, Compound 62, Compound 63

### Step 1: 3-[[4-[(2R)-2-[[2-(tert-Butoxycarbonylamino)spiro[3.3]heptan-6-yl]amino]-4,4-dimethyl-pentoxy]-6-(2,6-dimethylphenyl)-5-methyl-pyrimidin-2-yl]sulfamoyl]benzoic acid

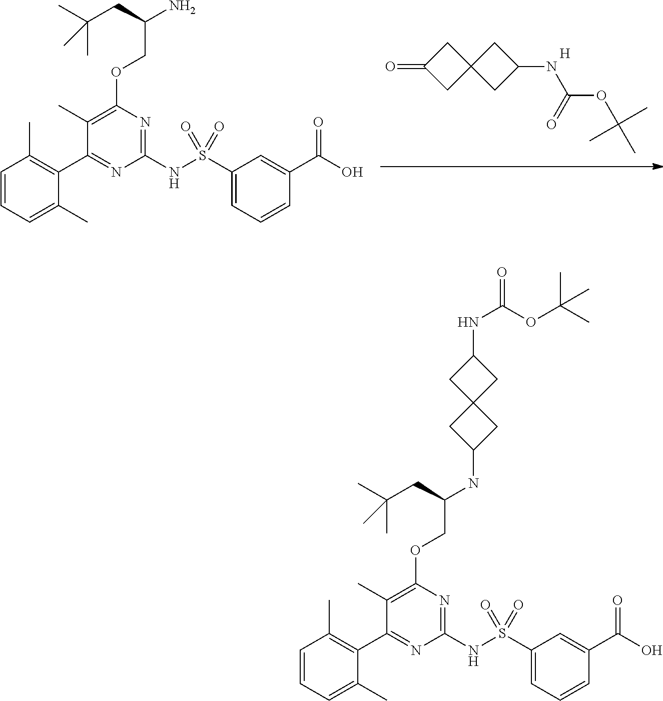

A 4 mL vial was charged with crude 3-[[4-[(2R)-2-amino-4,4-dimethyl-pentoxy]-6-(2,6-dimethylphenyl)-5-methyl-pyrimidin-2-yl]sulfamoyl]benzoic acid (hydrochloride salt) (309 mg, 0.5487 mmol) (80% pure), tert-butyl N-(2-oxospiro[3.3]heptan-6-yl)carbamate (148 mg, 0.6569 mmol), anhydrous DCM (0.90 mL) and sodium triacetoxyborohydride (350 mg, 1.651 mmol). The vial was briefly purged with nitrogen and the mixture was stirred at rt for 3 hours at which time LCMS showed 87% conversion. Another amount (98 mg) of borohydride was added and the mixture was stirred for an additional 1.5 hours. A bit of methanol and water were added (100 μL each) and the mixture was concentrated and dissolved in DMSO (total final volume 7.5 mL). The mixture was purified by reverse phase preparative HPLC (Cis) using a gradient of acetonitrile in water (1 to 99% over 15 min) and HCl as a modifier. The pure fractions were collected, and the organic solvent evaporated. A bit of brine was added to the aqueous phase and the solid that started to crash out was extracted with ethyl acetate (2×30 mL-no product detected in the aqueous phase). After drying over sodium sulfate, the organic solvent was evaporated to give 3-[[4-[(2R)-2-[[2-(tert-butoxycarbonylamino)spiro[3.3]heptan-6-yl]amino]-4,4-dimethyl-pentoxy]-6-(2,6-dimethylphenyl)-5-methyl-pyrimidin-2-yl]sulfamoyl]benzoic acid (232 mg, 57%) as a white solid. ESI-MS m/z calc. 735.3666, found 736.57 (M+1)⁺; Retention time: 1.68 minutes (LC method A).

### Step 2: tert-Butyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate, diastereomer 1 (Compound 59) and tert-butyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate, diastereomer 2 (Compound 60)

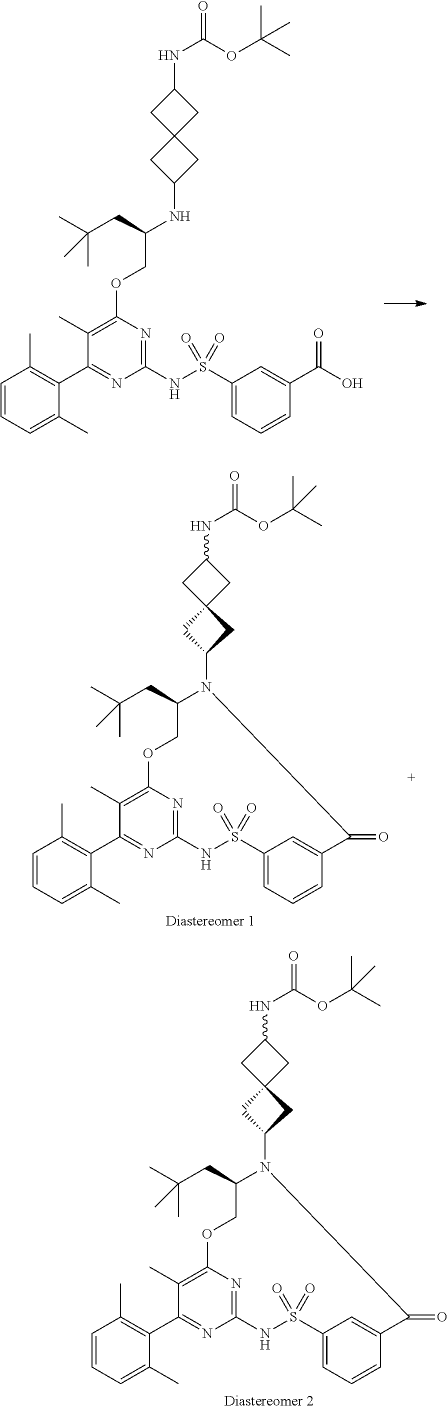

A 100 mL flask was charged under nitrogen with HATU (192 mg, 0.5050 mmol), anhydrous DMF (12 mL) and DIEA (0.28 mL, 1.608 mmol). A solution of 3-[[4-[(2R)-2-[[2-(tert-butoxycarbonylamino)spiro[3.3]heptan-6-yl]amino]-4,4-dimethyl-pentoxy]-6-(2,6-dimethylphenyl)-5-methyl-pyrimidin-2-yl]sulfamoyl]benzoic acid (232 mg, 0.3152 mmol) in anhydrous DMF (10 mL) was added dropwise through syringe over a period of 10 minutes. The mixture was stirred at room temperature for 3 days. The mixture was concentrated and diluted with DMSO (2 mL). The solution was microfiltered through a syringe filter disc and purified by reverse phase preparative HPLC (Cis) using a gradient of acetonitrile in water (1 to 99% over 15 min) and HCl as a modifier. Evaporation and trituration in DCM/hexanes gave tert-butyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]spiro[3.3]heptan-6-yl]carbamate (140 mg, 62%) as a white solid. ESI-MS m/z calc. 717.356, found 718.38 (M+1)⁺; Retention time: 2.19 minutes. second diastereomer visible Rt=2.20 minutes (ratio 1:1) (LC method A).

The two diastereomers were separated by chiral SFC using a phenomenex LUX-4 column (250×21.2 mm), 5 μM, 40° C.; mobile phase: 32% MeOH (no modifier), 68% CO₂, flow: 70 mL/min, concentration: 31 mg/mL in methanol:DMSO (91:9, no modifier), injection volume 500 μL, 150 bar, wavelength: 210 mm. For each isomer, the solvents were evaporated and the residue triturated in DCM/hexanes. Evaporation of the solvents gave the following compounds as white solids: Diastereomer 1, SFC peak 1, tert-butyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (74 mg, 64%). ESI-MS m/z calc. 717.356, found 718.79 (M+1)⁺; Retention time: 2.19 minutes (LC method A); and diastereomer 2, SFC peak 2, tert-butyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (66 mg, 57%), ESI-MS m/z calc. 717.356, found 718.79 (M+1)⁺; Retention time: 2.17 minutes (LC method A).

### Step 3: (11R)-12-(6-aminospiro[3.3]heptan-2-yl)-6-(2,6-dimethylphenyl)-11-(2,2-dimethylpropyl)-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 2 (Compound 63)

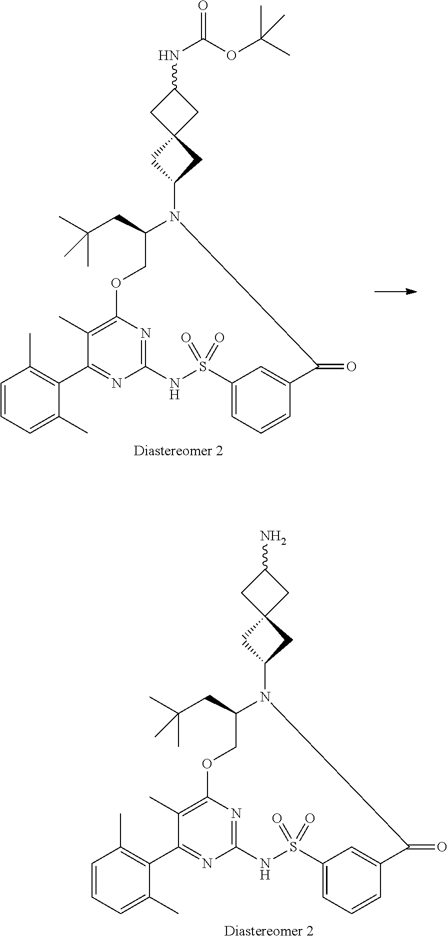

A 100 mL flask containing tert-butyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (63 mg, 0.08600 mmol) (SFC peak 2 diastereomer 2) was treated with DCM (0.9 mL) and HCl (0.63 mL of 4 M, 2.520 mmol) (4M in dioxane) at room temperature for 40 min. More HCl (0.5 mL) was added and the mixture was stirred for an additional 15 minutes. The volatiles were removed. The residue was treated with DCM/hexanes and the solvents were removed by evaporation. The operation was repeated several times until a white solid was obtained. Drying in vacuo gave (11R)-12-(6-aminospiro[3.3]heptan-2-yl)-6-(2,6-dimethylphenyl)-11-(2,2-dimethylpropyl)-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one (hydrochloride salt) (74 mg, 125%) as an off-white solid. ESI-MS m/z calc. 617.3036, found 618.69 (M+1)⁺; Retention time: 1.36 minutes (LC method A).

### Step 4: Methyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (Compound 61) and isopropyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (Compound 62)

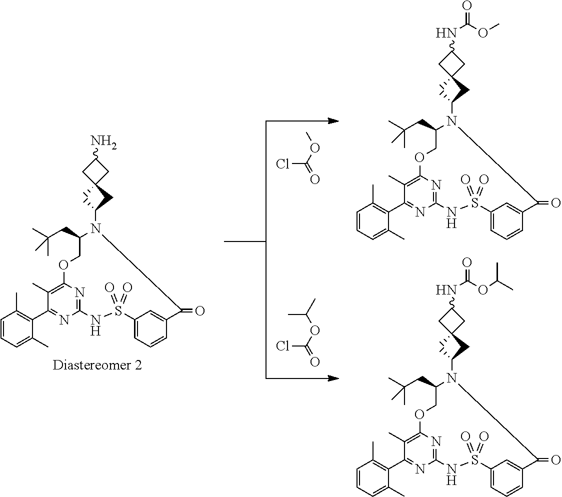

For each reaction, a 4 mL vial was charged with (11R)-12-(6-aminospiro[3.3]heptan-2-yl)-6-(2,6-dimethylphenyl)-11-(2,2-dimethylpropyl)-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 2 (hydrochloride salt) (12 mg, 0.01742 mmol), DCM (0.50 mL) and DIEA (15 μL, 0.08612 mmol). Methyl chloroformate (4 μL, 0.05177 mmol) (reaction A) or isopropyl chloroformate (18 μL of 2 M, 0.03600 mmol) (2M toluene solution, reaction B) was added and the mixture was stirred at room temperature for 2 hours. A bit of methanol was added, and the solvents were evaporated. DMSO (1 mL) was added. The solution was microfiltered through a syringe filter disc and purified by reverse phase preparative HPLC (Cis) using a gradient of acetonitrile in water (1 to 99% over 15 min) and HCl as a modifier to give, separately, methyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (6.6 mg, 55%), ESI-MS m/z calc. 675.3091, found 676.51 (M+1)⁺; Retention time: 1.87 minutes (LC method A); and isopropyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (6.4 mg, 51%). ESI-MS m/z calc. 703.34033, found 704.56 (M+1)⁺; Retention time: 2.05 minutes (LC method A).

### Example 31: Preparation of Compound 64 and Compound 65

### Step 1: Methyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (Compound 64) and isopropyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (Compound 65)

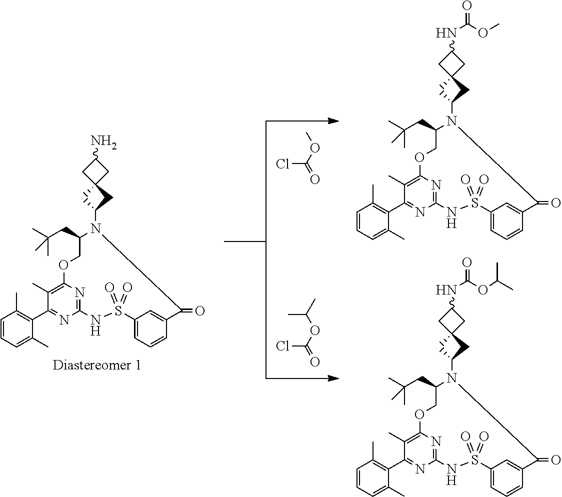

For each reaction, a 4 mL vial was charged with (11R)-12-(6-aminospiro[3.3]heptan-2-yl)-6-(2,6-dimethylphenyl)-11-(2,2-dimethylpropyl)-7-methyl-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 1 (hydrochloride salt) (prepared by a method analogous to that above, 12 mg, 0.01742 mmol), DCM (0.50 mL) and DIEA (15 μL, 0.08612 mmol). Methyl chloroformate (4 μL, 0.05177 mmol) (reaction A) or isopropyl chloroformate (18 μL of 2 M, 0.03600 mmol) (2M toluene solution, reaction B) was added and the mixture was stirred at room temperature for 1.5 hours. A bit of methanol was added, and the solvents were evaporated. DMSO (1 mL) was added. The solution was microfiltered through a syringe filter disc and purified by reverse phase preparative HPLC (Cis) using a gradient of acetonitrile in water (1 to 99% over 15 min) and HCl as a modifier to give, separately, methyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (5.7 mg, 47%), ESI-MS m/z calc. 675.3091, found 676.48 (M+1)⁺; Retention time: 1.87 minutes (LC method A); and isopropyl N-[2-[(11R)-6-(2,6-dimethylphenyl)-11-(2,2-dimethylpropyl)-7-methyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (6.6 mg, 53%). ESI-MS m/z calc. 703.34033, found 704.56 (M+1)⁺; Retention time: 2.07 minutes (LC method A).

### Example 32: Characterization of Compounds 66-103

The compounds in Table 7 were prepared in a manner analogous to that described above using commercially available reagents and intermediates described herein.

TABLE 7

| Compound | | LCMS | | | LCMS |
|---|---|---|---|---|---|
| Number | Structure | Rt (min) | Calc. mass | M + 1 | Method |
| 66 | 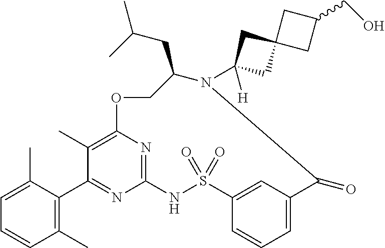 | 1.75 | 618.288 | 619.4 | A |
| 67 | 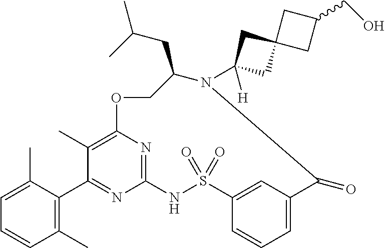 | 1.77 | 618.288 | 619.5 | A |
| 68 | 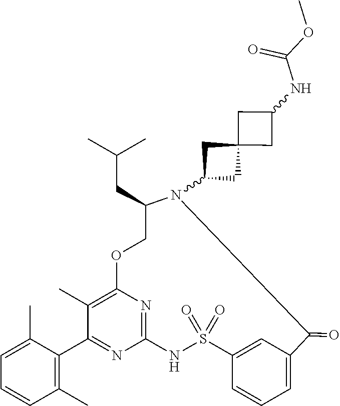 | 1.84 | 661.293 | 662.6 | A |
| 69 | 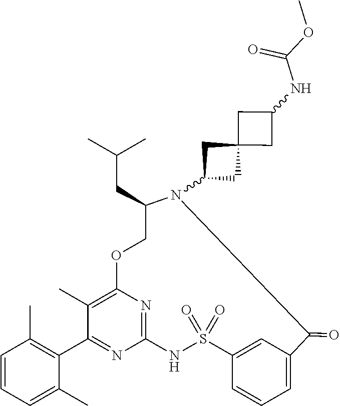 | 1.85 | 661.293 | 662.6 | A |
| 70 | 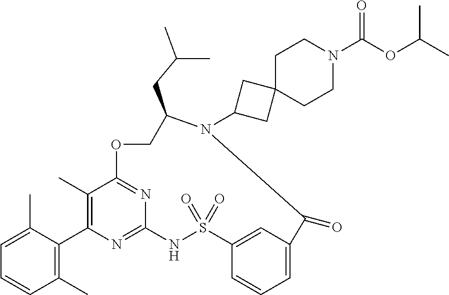 [SMILES: Cc1cccc(C)c1-c1nc2nc(c1C)OC[C@@H](CC(C)C)N(C1CC3(CCN(C(=O)OC(C)C)CC3)C1)C(=O)c1cccc(c1)S(=O)(=O)N2] | 2.11 | 703.34 | 704.5 | A |
| 71 | 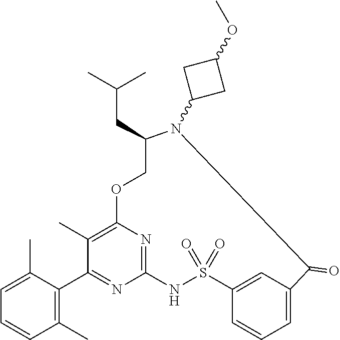 [SMILES: COC1CC(N2C(=O)c3cccc(c3)S(=O)(=O)Nc3nc(c(C)c(-c4c(C)cccc4C)n3)OC[C@H]2CC(C)C)C1] | 1.83 | 578.256 | 579.3 | A |
| 72 | 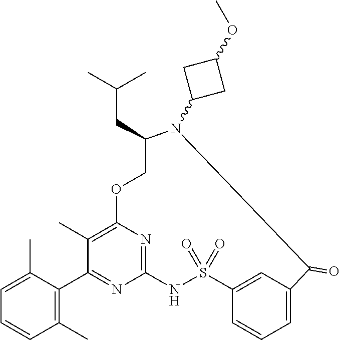 [SMILES: COC1CC(N2C(=O)c3cccc(c3)S(=O)(=O)Nc3nc(c(C)c(-c4c(C)cccc4C)n3)OC[C@H]2CC(C)C)C1] | 1.82 | 578.256 | 579.5 | A |
| 73 | 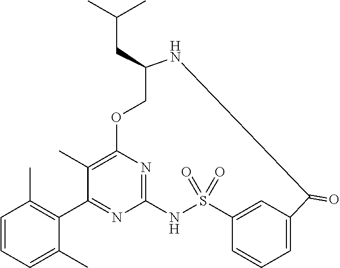 [SMILES: Cc1cccc(C)c1-c1nc2nc(c1C)OC[C@@H](CC(C)C)NC(=O)c1cccc(c1)S(=O)(=O)N2] | 1.52 | 494.199 | 495.3 | A |
| 74 | 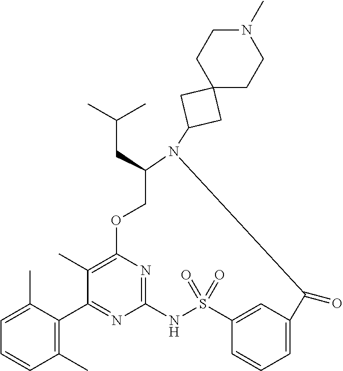 [SMILES: Cc1cccc(C)c1-c1nc2nc(c1C)OC[C@@H](CC(C)C)N(C1CC3(CCN(C)CC3)C1)C(=O)c1cccc(c1)S(=O)(=O)N2] | 1.32 | 631.319 | 632.6 | A |
| 75 | 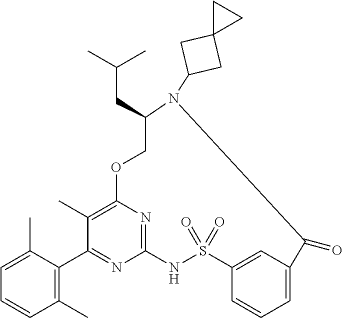 [SMILES: Cc1cccc(C)c1-c1nc2nc(c1C)OC[C@@H](CC(C)C)N(C1CC3(CC3)C1)C(=O)c1cccc(c1)S(=O)(=O)N2] | 2.09 | 574.261 | 575.3 | A |
| 56 | 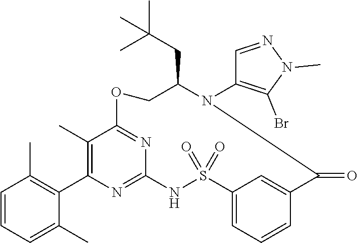 [SMILES: Cc1cccc(C)c1-c1nc2nc(c1C)OC[C@@H](CC(C)(C)C)N(c1cnn(C)c1Br)C(=O)c1cccc(c1)S(=O)(=O)N2] | 3.07 | 666.162 | 667.3 | 1D |
| 76 | 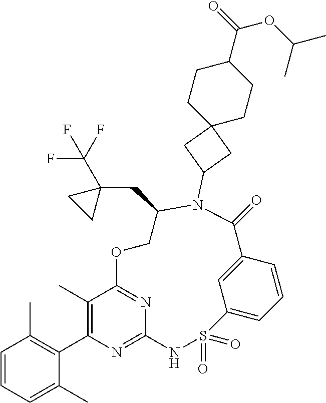 [SMILES: Cc1cccc(C)c1-c1nc2nc(c1C)OC[C@@H](CC1(C(F)(F)F)CC1)N(C1CC3(CCC(C(=O)OC(C)C)CC3)C1)C(=O)c1cccc(c1)S(=O)(=O)N2] | 2.08 | 769.312 | 770.2 | A |
| 77 | 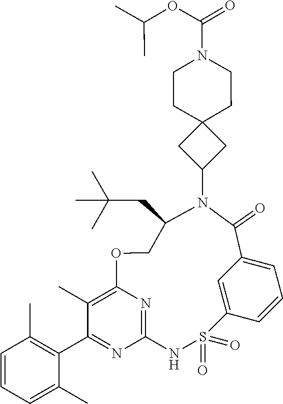 [SMILES: Cc1cccc(C)c1-c1nc2nc(c1C)OC[C@@H](CC(C)(C)C)N(C1CC3(CCN(C(=O)OC(C)C)CC3)C1)C(=O)c1cccc(c1)S(=O)(=O)N2] | 2.2 | 717.356 | 718.2 | A |
| 78 | 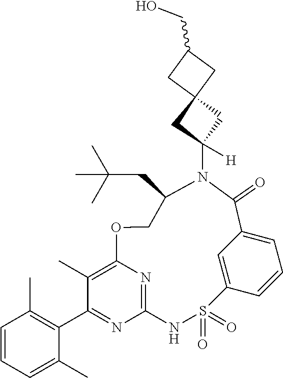 | 1.83 | 632.303 | 633.1 | A |
| 79 | 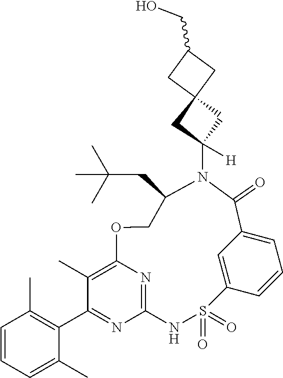 | 1.81 | 632.303 | 633.2 | A |
| 80 | 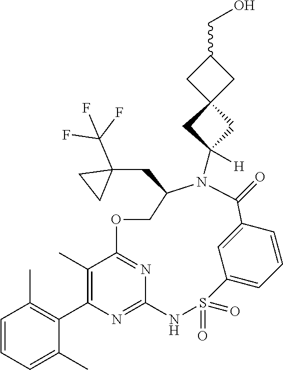 | 1.74 | 684.259 | 685.1 | A |
| 81 | 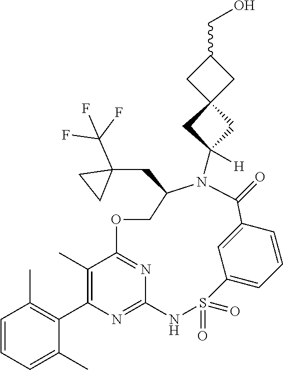 | 1.72 | 684.259 | 685.2 | A |
| 82 | 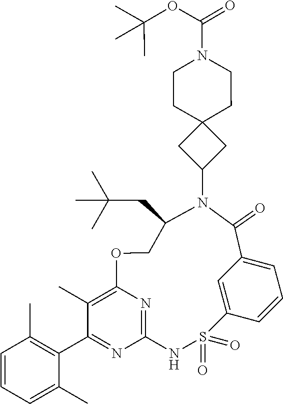 [SMILES: Cc1cccc(C)c1-c1nc2nc(c1C)OC[C@@H](CC(C)(C)C)N(C1CC3(CCN(C(=O)OC(C)(C)C)CC3)C1)C(=O)c1cccc(c1)S(=O)(=O)N2] | 2.3 | 731.372 | 732.3 | A |
| 83 | 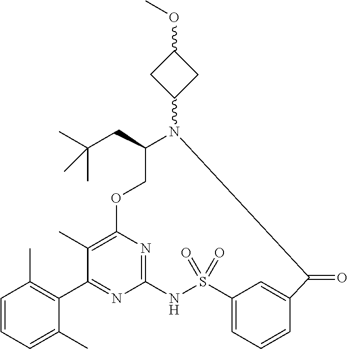 [SMILES: COC1CC(N2C(=O)c3cccc(c3)S(=O)(=O)Nc3nc(c(C)c(-c4c(C)cccc4C)n3)OC[C@H]2CC(C)(C)C)C1] | 1.97 | 592.272 | 593.3 | A |
| 84 | 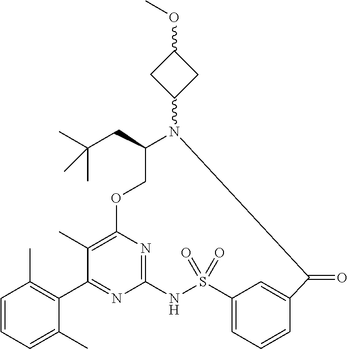 [SMILES: COC1CC(N2C(=O)c3cccc(c3)S(=O)(=O)Nc3nc(c(C)c(-c4c(C)cccc4C)n3)OC[C@H]2CC(C)(C)C)C1] | 1.96 | 592.272 | 593.3 | A |
| 85 | 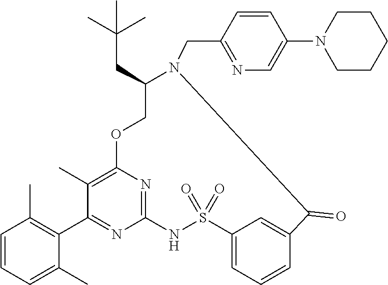 [SMILES: Cc1cccc(C)c1-c1nc2nc(c1C)OC[C@@H](CC(C)(C)C)N(Cc1ccc(N3CCCCC3)cn1)C(=O)c1cccc(c1)S(=O)(=O)N2] | 1.47 | 682.33 | 683.57 | A |
| 86 | 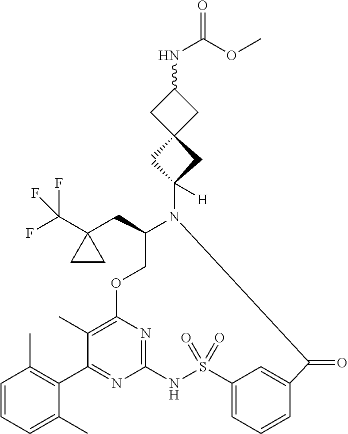 | 1.77 | 727.265 | 728.43 | A |
| 87 | 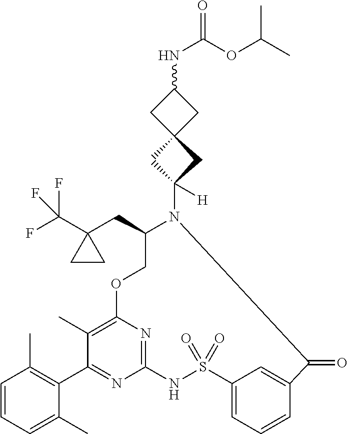 | 1.94 | 755.296 | 756.44 | A |
| 88 | 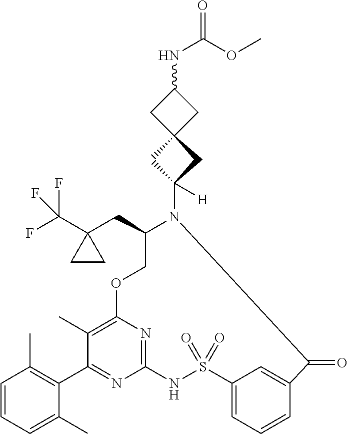 | 1.78 | 727.265 | 728.47 | A |
| 89 | 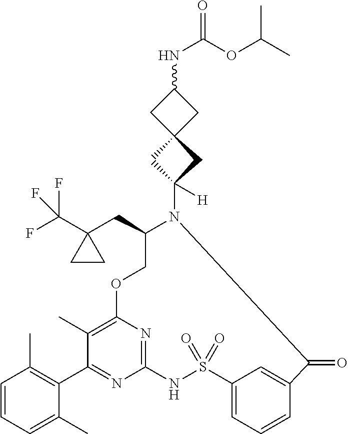 | 1.95 | 755.296 | 756.48 | A |
| 90 | 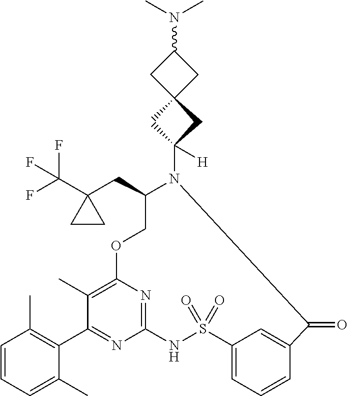 | 1.35 | 697.291 | 698.69 | A |
| 91 | 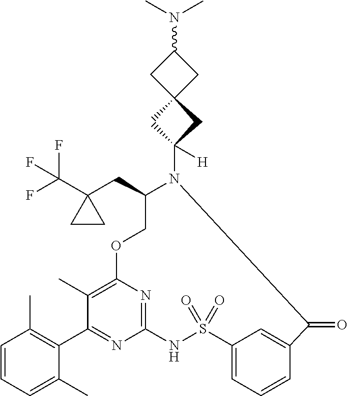 | 1.37 | 697.291 | 698.73 | A |
| 92 | 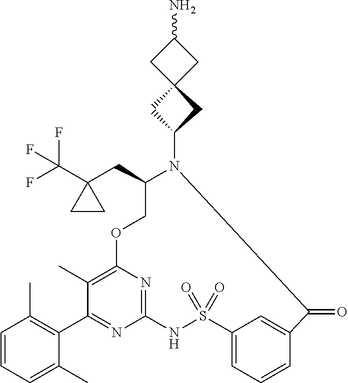 | 1.31 | 669.26 | 670.68 | A |
| 93 | 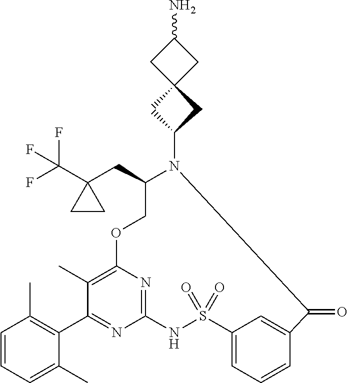 | 1.32 | 669.26 | 670.38 | A |
| 94 | 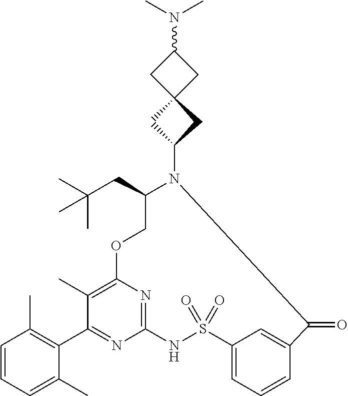 | 1.4 | 645.335 | 646.7 | A |
| 95 | 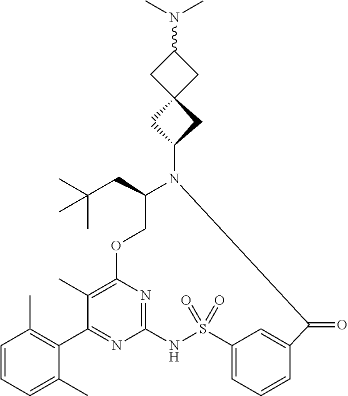 | 1.43 | 645.335 | 646.78 | A |
| 63 | 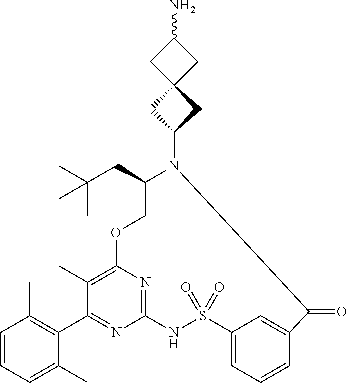 | 1.36 | 617.304 | 618.69 | A |
| 96 | 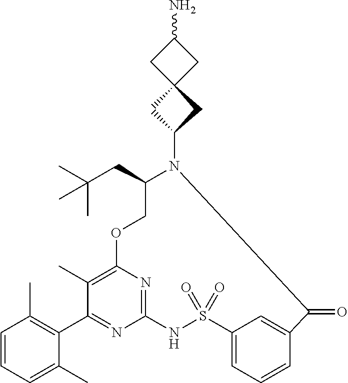 | 1.37 | 617.304 | 618.35 | A |
| 97 | 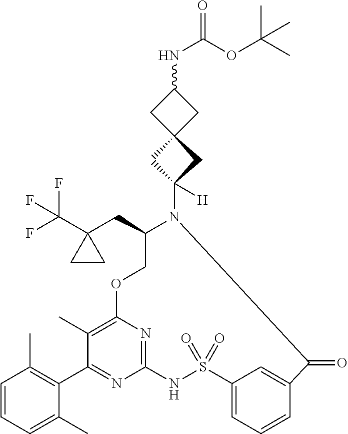 | 2.06 | 769.312 | 770.52 | A |
| 98 | 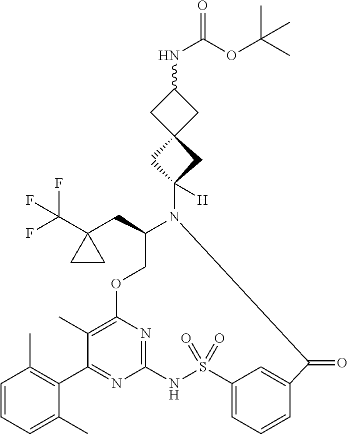 | 2.05 | 769.312 | 770.48 | A |
| 99 | 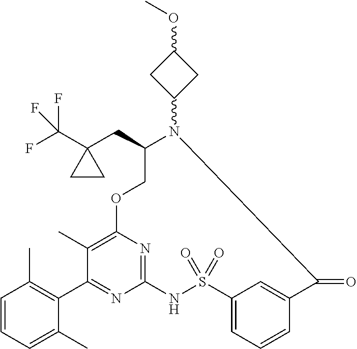 [SMILES: COC1CC(N2C(=O)c3cccc(c3)S(=O)(=O)Nc3nc(c(C)c(-c4c(C)cccc4C)n3)OC[C@H]2CC2(C(F)(F)F)CC2)C1] | 1.77 | 644.228 | 645.99 | A |
| 100 | 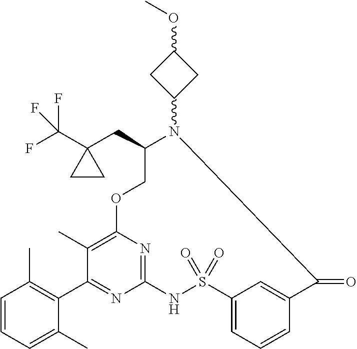 [SMILES: COC1CC(N2C(=O)c3cccc(c3)S(=O)(=O)Nc3nc(c(C)c(-c4c(C)cccc4C)n3)OC[C@H]2CC2(C(F)(F)F)CC2)C1] | 1.75 | 644.228 | 645.39 | A |
| 101 | 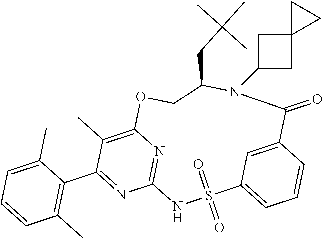 [SMILES: Cc1cccc(C)c1-c1nc2nc(c1C)OC[C@@H](CC(C)(C)C)N(C1CC3(CC3)C1)C(=O)c1cccc(c1)S(=O)(=O)N2] | 2.11 | 588.277 | 589.4 | A |
| 102 | 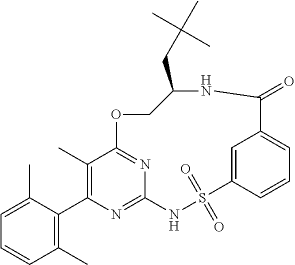 [SMILES: Cc1cccc(C)c1-c1nc2nc(c1C)OC[C@@H](CC(C)(C)C)NC(=O)c1cccc(c1)S(=O)(=O)N2] | 1.54 | 508.214 | 509.3 | A |
| 103 | 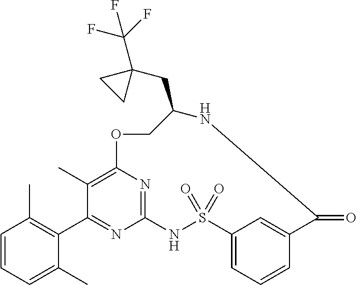 [SMILES: Cc1cccc(C)c1-c1nc2nc(c1C)OC[C@@H](CC1(C(F)(F)F)CC1)NC(=O)c1cccc(c1)S(=O)(=O)N2] | 1.5 | 560.171 | 561.3 | A |

TABLE 8

| Compound | |
|---|---|
| Number | NMR |
| 56 | ¹H NMR (400 MHz, DMSO-d₆) δ 13.05 (br s, 1H), 8.77 (br s, 1H), 8.00 (br s, 1H), |
| | 7.83 (s, 1H), 7.70 (br s, 2H), 7.27 (br s, 1H), 7.18 (d, J = 8.0 Hz, 1H), 7.12 (d, J = |
| | 8.0 Hz, 1H), 5.45 (br s, 1H), 4.04 (br s, 1H), 3.96-3.88 (m, 4H), 2.06 (br s, |
| | 3H), 1.77 (br s, 3H), 1.70-1.61 (m, 1H), 1.57 (br s, 3H), 1.25 (d, J = 15.2 Hz, |
| | 1H), 0.55 (s, 9H). |
| 85 | ¹H NMR (400 MHz, DMSO-d₆) δ 13.04 (broad s, 1H), 8.63 (s, 1H), 8.33 (s, 1H), |
| | 8.06-7.81 (m, 2H), 7.70 (br s, 3H), 7.28 (t, J = 7.6 Hz, 1H), 7.18 (d, J = 7.6 Hz, |
| | 1H), 7.13 (d, J = 7.6 Hz, 1H), 5.33 (dd, J = 10.6, 4.5 Hz, 1H), 4.86 (d, J = 15.5 |
| | Hz, 1H), 4.70 (d, J = 15.7 Hz, 1H), 4.43 (t, J = 11.2 Hz, 1H), 4.12-3.99 (m, 1H), |
| | 3.36 (d, J = 10.0 Hz, 4H), 2.06 (s, 3H), 1.86-1.72 (m, 4H), 1.70-1.53 (m, 9H), |
| | 1.39 (d, J = 15.2 Hz, 1H), 0.51 (s, 9H). |

### Example 33: Preparation of Compound 104

### Step 1: 3-[[4-[(3R,4R)-4-amino-1-tert-butoxycarbonyl-pyrrolidin-3-yl]oxy-6-(2,6-dimethylphenyl)-5-methyl-pyrimidin-2-yl]sulfamoyl]benzoic acid

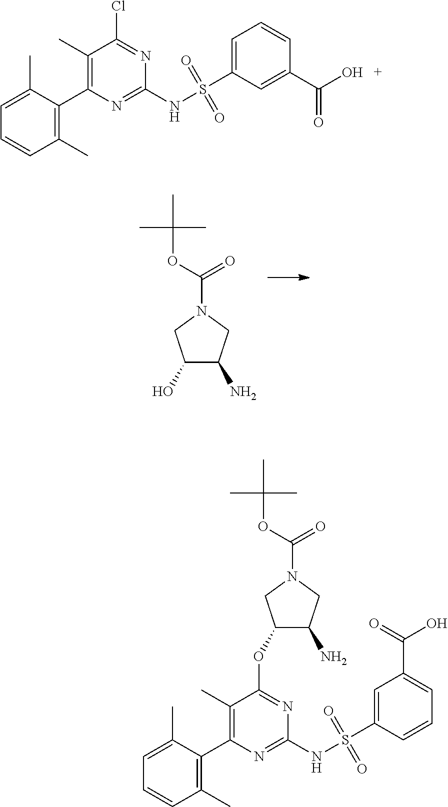

A solution of 3-[[4-chloro-6-(2,6-dimethylphenyl)-5-methyl-pyrimidin-2-yl]sulfamoyl]benzoic acid (0.43 g, 0.9956 mmol), tert-butyl (3R,4R)-3-amino-4-hydroxy-pyrrolidine-1-carboxylate (0.20 g, 0.9889 mmol), and sodium tert-butoxide (0.28 g, 2.914 mmol) in THF (5 mL) was stirred for 23 hours. The reaction was acidified with 1 M citric acid, diluted with water, and extracted with ethyl acetate. The combined extracts were washed with brine, dried over sodium sulfate, and evaporated under vacuum. The residue was purified by silica gel column chromatography with 0-10% methanol in dichloromethane to give 3-[[4-[(3R,4R)-4-amino-1-tert-butoxycarbonyl-pyrrolidin-3-yl]oxy-6-(2,6-dimethylphenyl)-5-methyl-pyrimidin-2-yl]sulfamoyl]benzoic acid (0.55 g, 92%) ESI-MS m/z calc. 597.2257, found 598.3 (M+1)⁺; Retention time: 0.51 minutes as a colorless solid. LC method D.

### Step 2: (3R,7R)-19-(2,6-Dimethylphenyl)-20-methyl-2-oxa-15×⁶-thia-5,8,16,18,21-pentaazatetracyclo[15.3.1.110,14.03,7]docosa-1(21),10,12,14(22),17,19-hexaene-9,15,15-trione

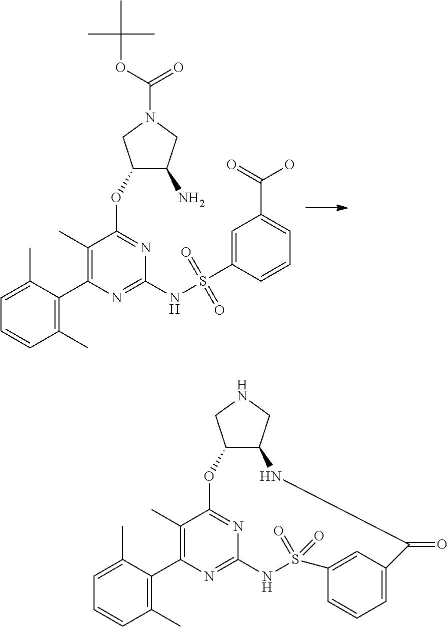 [SMILES: Cc1cccc(C)c1-c1nc(NS(=O)(=O)c2cccc(C(=O)O)c2)nc(O[C@@H]2CN(C(=O)OC(C)(C)C)C[C@H]2N)c1C ; Cc1cccc(C)c1-c1nc2nc(c1C)O[C@@H]1CNC[C@H]1NC(=O)c1cccc(c1)S(=O)(=O)N2]

A solution of 3-[[4-[(3R,4R)-4-amino-1-tert-butoxycarbonyl-pyrrolidin-3-yl]oxy-6-(2,6-dimethylphenyl)-5-methyl-pyrimidin-2-yl]sulfamoyl]benzoic acid (0.20 g, 0.3346 mmol), [[(E)-(1-cyano-2-ethoxy-2-oxo-ethylidene)amino]oxy-tetrahydropyran-4-yl-methylene]-dimethyl-ammonium (Phosphorus Hexafluoride Ion) (0.22 g, 0.5149 mmol), and N,N-diisopropylethylamine (0.18 mL, 1.033 mmol) in DMF (17 mL) was stirred for 22 hours. The reaction was acidified with 1 M citric acid, diluted with water, and extracted with ethyl acetate. The combined extracts were washed with brine, dried over sodium sulfate, and evaporated under vacuum. The residue was purified by silica gel column chromatography with 0-6% methanol in dichloromethane to give tert-butyl (3R,7R)-19-(2,6-dimethylphenyl)-20-methyl-9,15,15-trioxo-2-oxa-15λ⁶-thia-5,8,16,18,21-pentaazatetracyclo[15.3.1.110,14.03,7]docosa-1(21),10,12,14(22),17,19-hexaene-5-carboxylate (78 mg, 40%) as a tan solid. The solid was stirred with HCl (3 mL of 4 M, 12.00 mmol) (in dioxane) for 30 minutes. The solvent was removed under vacuum, and the solids were triturated with diethyl ether to give (3R,7R)-19-(2,6-dimethylphenyl)-20-methyl-2-oxa-15λ⁶-thia-5,8,16,18,21-pentaazatetracyclo[15.3.1.110,14.03,7]docosa-1(21),10,12,14(22),17,19-hexaene-9,15,15-trione (hydrochloride salt) (62 mg, 36%) ESI-MS m/z calc. 479.16272, found 480.2 (M+1)⁺; Retention time: 0.32 minutes; LC method D.

### Step 3: (3R,7R)-19-(2,6-Dimethylphenyl)-20-methyl-5-{spiro[3.5]nonan-2-yl}-2-oxa-15)⁶-thia-5,8,16,18,21-pentaazatetracyclo[15.3.1.110,14.03,7]docosa-1(21),10,12,14(22),17,19-hexaene-9,15,15-trione (Compound 104)

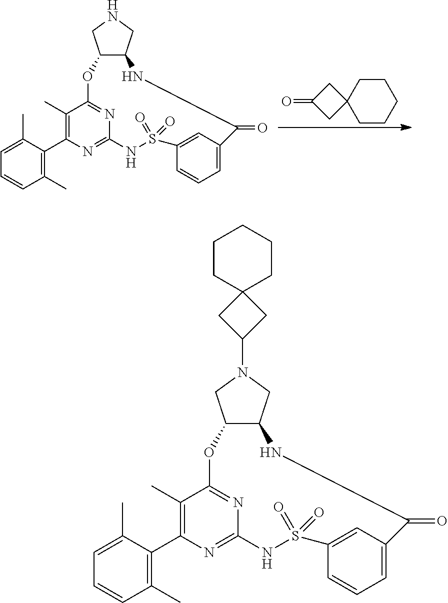

A solution of (3R,7R)-19-(2,6-dimethylphenyl)-20-methyl-2-oxa-15?⁶-thia-5,8,16,18,21-pentaazatetracyclo[15.3.1.110,14.03,7]docosa-1(21),10,12,14(22),17,19-hexaene-9,15,15-trione (hydrochloride salt) (30 mg, 0.05814 mmol), spiro[3.5]nonan-2-one (17 mg, 0.1230 mmol), and sodium triacetoxyborohydride (38 mg, 0.1793 mmol) in dichloromethane (0.3 mL) was stirred for three hours. The reaction was stirred with methanol, and the solvents were evaporated. The residue was purified by reverse-phase HPLC-MS (1%-99% acetonitrile/water (5 mM HCl)) to give (3R,7R)-19-(2,6-dimethylphenyl)-20-methyl-5-{spiro[3.5]nonan-2-yl}-2-oxa-15λ⁶-thia-5,8,16,18,21-pentaazatetracyclo[15.3.1.110,14.03,7]docosa-1(21),10,12,14(22),17,19-hexaene-9,15,15-trione (hydrochloride salt) (14.5 mg, 39%) ESI-MS m/z calc. 601.2723, found 602.5 (M+1)⁺; Retention time: 1.32 minutes, LC method A.

### Example 34: Preparation of Compound 105

### Step 1: Benzyl 2-[(4R)-2-oxooxazolidin-4-yl]acetate

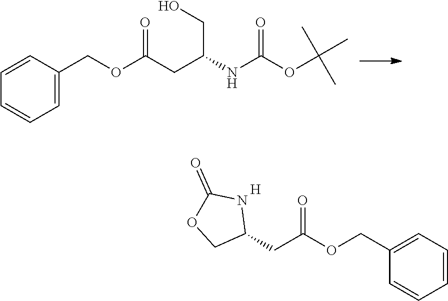

To a solution of benzyl (3R)-3-(tert-butoxycarbonylamino)-4-hydroxy-butanoate (27.8 g, 89.864 mmol)benzyl (3R)-3-(tert-butoxycarbonylamino)-4-hydroxy-butanoate (27.8 g, 89.864 mmol) in 1,2-dichloroethane (250 mL) was added pyridine (65.526 g, 67 mL, 828.40 mmol) and the mixture was cooled to 0-5° C. p-toluenesulfonic anhydride (32.263 g, 98.850 mmol) was added and the mixture was warmed to room temperature and stirred for 2 hours and then heated to 90° C. for 2 hours. The mixture was cooled, diluted with dichloromethane (500 mL) and washed with 1N HCl (3×200 mL). The combined aqueous layers were backextracted with dichloromethane (2×150 mL). The combined organic layers were dried with sodium sulfate, filtered and concentrated to dryness. The crude material was purified by flash chromatography (330 g) using a gradient of 20% to 100% ethyl acetate in heptane to afford enantiopure benzyl 2-[(4R)-2-oxooxazolidin-4-yl]acetate (18.11 g, 86%) as a white solid. ¹H NMR (400 MHz, CDCl₃) δ 7.44-7.31 (m, 5H), 5.58 (br. s., 1H), 5.16 (s, 2H), 4.56 (t, J=8.6 Hz, 1H), 4.25 (qd, J=7.0, 5.9 Hz, 1H), 4.06 (dd, J=8.9, 5.7 Hz, 1H), 2.76-2.63 (m, 2H). ESI-MS m/z calc. 235.0845, found 236.2 (M+1)⁺, 471.2 (2M+H)⁺; Retention time: 1.49 minutes; LC method X.

### Step 2: (4R)-4-(2-Hydroxy-2-methyl-propyl)oxazolidin-2-one

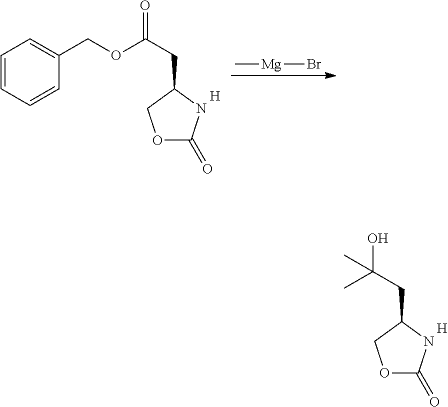

Bromo(methyl)magnesium in diethyl ether (105 mL of 3 M, 315.00 mmol) was added to a mixture of toluene (150 mL) and THF (150 mL) at −20° C. A warm THF (80 mL) solution of benzyl 2-[(4R)-2-oxooxazolidin-4-yl]acetate (18.1 g, 76.944 mmol) was then added dropwise maintaining the temperature below −10° C. The mixture was warm up to room temperature and stirred for 18 hours. The mixture was added via canula to a solution of acetic acid (85 mL) in water (440 mL) at 0° C. The resultant mixture was stirred for 1 hour at room temperature. The layers were separated. The aqueous layer was saturated with brine (200 mL) and further extracted with 2-methyltetrahydrofuran (3×250 mL) and with ethanol/chloroform (½, 3×330 mL). The combined organic extracts were dried over anhydrous sodium sulfate, filtered and concentrated. The residue was co-evaporated with heptanes (4×100 mL). The crude material was purified in two equal batches by flash chromatography (330 g) eluting with 6% isopropanol in dichloromethane) to give (4R)-4-(2-hydroxy-2-methyl-propyl)oxazolidin-2-one (8.88 g, 69%) as an off-white solid. ¹H NMR (400 MHz, DMSO-d₆) δ 7.36 (s, 1H), 4.45-4.38 (m, 1H), 4.36 (s, 1H), 4.00-3.91 (m, 2H), 1.68-1.54 (m, 2H), 1.10 (s, 6H). ESI-MS m/z calc. 159.0895, found 160.2 (M+1)⁺; Retention time: 0.77 minutes, LC method X.

### Step 3: (2R)-2-Amino-4-methyl-pentane-1,4-diol

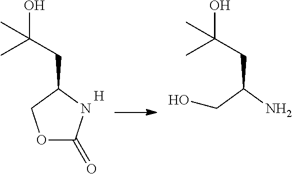

A mixture of (4R)-4-(2-hydroxy-2-methyl-propyl)oxazolidin-2-one (904 mg, 4.2592 mmol) and barium hydroxide octahydrate (4.03 g, 12.775 mmol) in ethanol (20 mL) and water (20 mL) was stirred at 90-95° C. for 4 hours. After cooling down to room temperature, dry ice (˜7 g) was added and the mixture was stirred vigorously for 2 days. The suspension was filtered over a Celite pad and rinsed with ethanol (20 mL). The filtrate was diluted with toluene and concentrated under reduced pressure to provide (2R)-2-amino-4-methyl-pentane-1,4-diol (780 mg) which was used without further purification for the next step. ¹H NMR (400 MHz, DMSO-d₆) δ 5.12 (br. s., 2H), 3.30-3.16 (m, 2H), 2.94 (dd, J=9.0, 3.4 Hz, 1H), 1.83 (s, 2H), 1.49-1.40 (m, 1H), 1.33-1.21 (m, 1H), 1.11 (d, J=11.0 Hz, 6H). ESI-MS m/z calc. 133.1103, found 134.4 (M+1)⁺; Retention time: 0.21 minutes, LC method X.

### Step 4: 3-[[4-[(2R)-2-Amino-4-hydroxy-4-methyl-pentoxy]-6-(2,6-dimethylphenyl)-5-methyl-pyrimidin-2-yl]sulfamoyl]benzoic acid

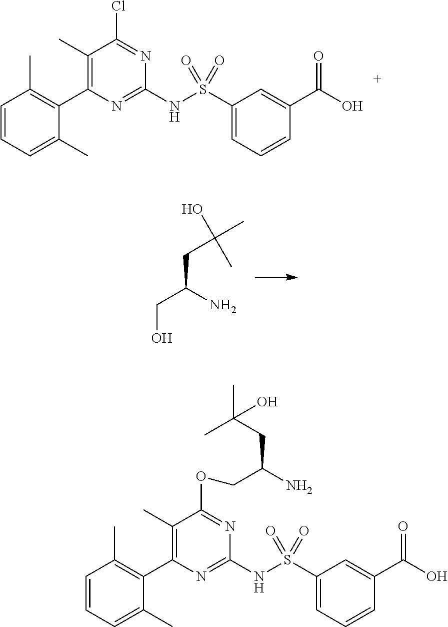

To a solution of (2R)-2-amino-4-methyl-pentane-1,4-diol (1.4 g, 10.511 mmol) and 3-[[4-chloro-6-(2,6-dimethylphenyl)-5-methyl-pyrimidin-2-yl]sulfamoyl]benzoic acid (3.7 g, 8.5669 mmol) in tetrahydrofuran (20 mL) was slowly added sodium tert-butoxide in tetrahydrofuran (17.2 mL of 2 M, 34.400 mmol) at 0° C. The mixture was stirred at room temperature for 5 hours. Sodium tert-butoxide in tetrahydrofuran (5 mL of 2 M, 10.000 mmol) was added and the mixture was stirred for additional 16 hours. The reaction was partitioned between ethyl acetate (100 mL) and 1 N hydrochloric acid (40 mL). The aqueous phase was extracted with ethyl acetate (2×20 mL) and 2-methyltetrahydrofuran (4×30 mL). The combined organic layers were dried over sodium sulfate, filtered and concentrated to dryness. The residue was triturated with ethyl acetate (30 mL), the precipitate was filtered, washed with ethyl acetate (2×20 mL), dissolved in MeOH (25 mL) and concentrated to dryness to afford 3-[[4-[(2R)-2-amino-4-hydroxy-4-methyl-pentoxy]-6-(2,6-dimethylphenyl)-5-methyl-pyrimidin-2-yl]sulfamoyl]benzoic acid (hydrochloride salt) (2.57 g, 53%) as a white solid. ¹H NMR (400 MHz, DMSO-d₆) δ 13.34 (br. s., 2H), 8.43 (s, 1H), 8.12 (d, J=6.6 Hz, 2H), 8.06-7.94 (m, 3H), 7.69 (t, J=7.7 Hz, 1H), 7.26 (d, J=7.8 Hz, 1H), 7.15 (d, J=7.1 Hz, 2H), 5.14 (br. s., 1H), 4.32 (d, J=9.0 Hz, 1H), 4.17 (dd, J=11.7, 7.3 Hz, 1H), 3.79 (br. s., 1H), 1.93 (br. s., 6H), 1.73 (d, J=5.9 Hz, 2H), 1.67 (br. s., 3H), 1.32-1.17 (m, 6H). ESI-MS m/z calc. 528.2043, found 529.2 (M+1)⁺; Retention time: 2.32 minutes, LC method Y.

### Step 5: (11R)-6-(2,6-Dimethylphenyl)-11-(2-hydroxy-2-methyl-propyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 105)

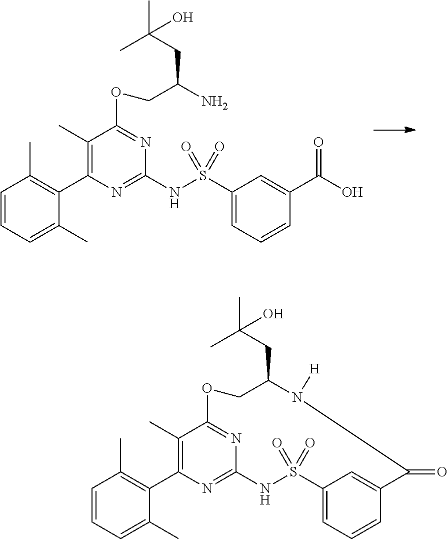 [SMILES: Cc1cccc(C)c1-c1nc(NS(=O)(=O)c2cccc(C(=O)O)c2)nc(OC[C@H](N)CC(C)(C)O)c1C ; Cc1cccc(C)c1-c1nc2nc(c1C)OC[C@@H](CC(C)(C)O)NC(=O)c1cccc(c1)S(=O)(=O)N2]

To solution of 3-[[4-[(2R)-2-amino-4-hydroxy-4-methyl-pentoxy]-6-(2,6-dimethylphenyl)-5-methyl-pyrimidin-2-yl]sulfamoyl]benzoic acid (hydrochloride salt) (100 mg, 0.1717 mmol) and triethylamine (108.90 mg, 0.15 mL, 1.0762 mmol) in ethyl acetate (7 mL) and DMF (1.5 mL) was added propylphosphonic anhydride solution in ethyl acetate (320 mg, 0.2993 mL, 0.5029 mmol) at 0° C. The ice bath was removed, and the mixture was stirred at room temperature for 1 hour. Ethyl acetate was removed under vacuo and the resulting mixture was purified by reverse phase chromatography (C₁₈ 50 g) using a gradient of 5% to 100% methanol in water to afford (11R)-6-(2,6-dimethylphenyl)-11-(2-hydroxy-2-methyl-propyl)-7-methyl-2,2-dioxo-9-oxa-2,6-thia-3,5,12,19-tetrazatricyclo[12.3.1.14,8]nonadeca-1(18),4(19),5,7,14,16-hexaen-13-one (67 mg, 76%) as white solid after lyophilization. ¹H NMR (taken at 80° C.) (400 MHz, DMSO-d₆) δ 12.05 (br. s., 1H), 8.57 (s, 1H), 7.93 (d, J=7.1 Hz, 1H), 7.69-7.59 (m, 2H), 7.47 (d, J=9.8 Hz, 1H), 7.29-7.21 (m, 1H), 7.13 (dd, J=19.1, 7.6 Hz, 2H), 5.15 (dd, J=10.8, 3.9 Hz, 1H), 4.03-3.61 (m, 2H), 3.55-3.43 (m, J=9.5 Hz, 1H), 2.05 (s, 3H), 1.82 (s, 3H), 1.78-1.67 (m, 2H), 1.64 (s, 3H), 0.82-0.79 (m, 6H). ESI-MS m/z calc. 510.1937, found 511.2 (M+1)⁺; Retention time: 3.14 minutes (LC method Y).

### Example 35: Preparation of Compound 106

### Step 1: Benzyl (4R)-4-(tert-butoxycarbonylamino)-5-hydroxy-pentanoate

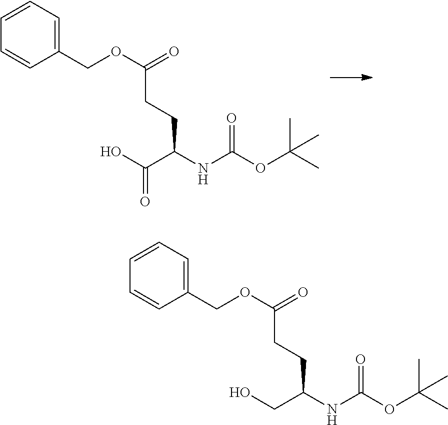

(2R)-5-Benzyloxy-2-(tert-butoxycarbonylamino)-5-oxo-pentanoic acid (10 g, 29.641 mmol) was dissolved in dimethoxyethane (30 mL) and the solution was cooled to −15° C. N-methylmorpholine (3.0360 g, 3.3 mL, 30.016 mmol) was added followed by a slow addition of isobutyl chloroformate (4.1067 g, 3.9 mL, 30.069 mmol) such that the reaction temperature was kept below −10° C. The mixture was stirred for 30 minutes. The solids were quickly filtered and washed with dimethoxyethane (30 mL). The filtrate was cooled to −40° C. and a solution of sodium borohydride (1.45 g, 38.327 mmol) in water (15 mL) was added slowly such that the reaction temperature was maintained between −30° C. and −15° C. The mixture was stirred for 15 minutes. Water (180 mL) was then added dropwise at −15° C. and the temperature was slowly raised to 5° C. while controlling the gas evolution. The suspension was filtered and washed with water (300 mL). The solid was dissolved in dichloromethane (100 mL) and transferred in a separatory funnel. Phases were separated, the organic phase was dried over sodium sulfate, filtered and evaporated to dryness to give benzyl (4R)-4-(tert-butoxycarbonylamino)-5-hydroxy-pentanoate (7.98 g, 83%) as a white solid. ¹H NMR (400 MHz, CDCl₃) δ 7.42-7.30 (m, 5H), 5.13 (s, 2H), 4.81 (br. s., 1H), 3.65 (br. s., 2H), 3.60-3.51 (m, 1H), 2.57-2.36 (m, 3H), 1.98-1.87 (m, 1H), 1.86-1.73 (m, 1H), 1.44 (s, 9H). ESI-MS m/z calc. 323.1733, found 224.4 (M−99)⁺; Retention time: 1.696 minutes, LC method X.

### Step 2: Benzyl 3-[(4R)-2-oxooxazolidin-4-yl]propanoate

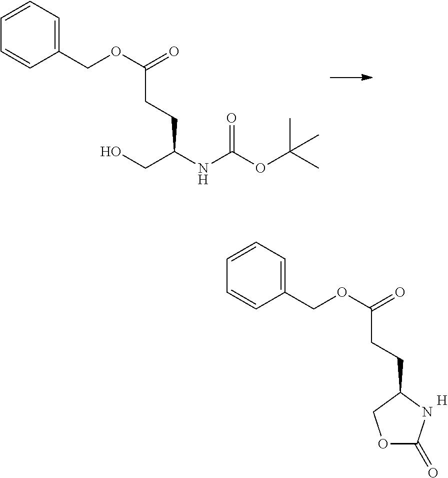

To a solution of benzyl (4R)-4-(tert-butoxycarbonylamino)-5-hydroxy-pentanoate (7.98 g, 24.652 mmol) in dichloroethane (80 mL) was added pyridine (48.900 g, 50 mL, 618.21 mmol). p-toluenesulfonic anhydride (8.65 g, 25.972 mmol) was then added and the mixture was stirred at room temperature for 1 hour and then heated to 90° C. for 2 hours. The mixture was cooled, diluted with dichloromethane (150 mL) and washed with 1N HCl (3×100 mL). The combined organic layers were washed with brine, dried with sodium sulfate and the solvents were removed in vacuo. The residue was purified by silica-gel column chromatography on a 80 g column, eluting from 20% to 80% of EtOAc in heptane to yield benzyl 3-[(4R)-2-oxooxazolidin-4-yl]propanoate (4.85 g, 77%) as a pale brown oil that slowly crystalized over time. ¹H NMR (400 MHz, CDCl₃) δ 7.43-7.30 (m, 5H), 6.15 (br. s., 1H), 5.13 (s, 2H), 4.48 (t, J=8.4 Hz, 1H), 4.02 (dd, J=8.6, 6.1 Hz, 1H), 3.97-3.88 (m, 1H), 2.45 (t, J=7.3 Hz, 2H), 2.00-1.85 (m, 2H). ESI-MS m/z calc. 249.1001, found 250.2 (M+1)⁺; Retention time: 1.511 minutes, LC method X.

### Step 3: (4R)-4-(3-Hydroxy-3-methyl-butyl)oxazolidin-2-one

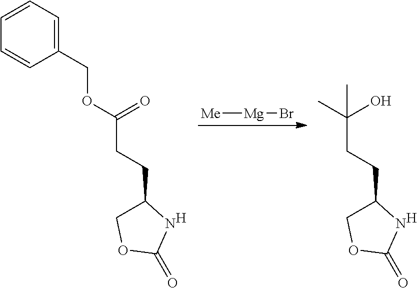

Methylmagnesium bromide (26 mL of 3 M, 78.000 mmol) in diethyl ether was added to a mixture of toluene (42 mL) and tetrahydrofuran (42 mL) at −20° C. (methanol+water+dried ice). A warm tetrahydrofuran (22 mL) solution of benzyl 3-[(4R)-2-oxooxazolidin-4-yl]propanoate (4.85 g, 19.457 mmol) was then added dropwise maintaining the temperature below −10° C. The mixture was warmed up to room temperature and stirred for 2 hours. The reaction mixture was cooled to 0° C., quenched with a 10% aqueous acetic acid solution (50 mL) and the resultant mixture was stirred for 1 hour at room temperature. The layers were separated. The aqueous layer was extracted with methyl-THF (3×100 mL) and then with dichloromethane (2×100 mL). The organic phases were combined, dried on anhydrous sodium sulfate, filtered and concentrated under reduced pressure. The residue was purified by silica-gel column chromatography on a 50 g and 120 g column, eluting from 0 to 15% of isopropanol in dichloromethane to afford (4R)-4-(3-hydroxy-3-methyl-butyl)oxazolidin-2-one (1.73 g, 51%) as a white solid. ¹H NMR (400 MHz, CDCl₃) δ 6.05 (br. s., 1H), 4.50 (t, J=8.4 Hz, 1H), 4.03 (dd, J=8.4, 6.2 Hz, 1H), 3.95-3.81 (m, 1H), 1.76-1.64 (m, 2H), 1.59-1.44 (m, 3H), 1.25 (s, 6H). ESI-MS m/z calc. 173.1052, found 174.2 (M+1)⁺; Retention time: 0.95 minutes, LC method X.

### Step 4: (2R)-2-Amino-5-methyl-hexane-1,5-diol

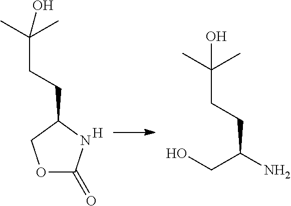

A mixture of (4R)-4-(3-hydroxy-3-methyl-butyl)oxazolidin-2-one (307 mg, 1.7724 mmol), barium hydroxide octahydrate (1.69 g, 5.3572 mmol), ethanol (12 mL) and water (12 mL) was heated at 95° C. to reflux for 2 hours. Reaction mixture was cooled to room temperature before dry ice was slowly added (˜1,8 g) and mixture was stirred vigorously for 2 days. The suspension was filtered over a Celite pad and rinsed with ethanol (˜15 mL). The filtrate was diluted with toluene, co-evaporated three times and concentrated under reduced pressure. Barium salts were observed on the walls of the flask. A minimum of ethanol was added, and the solution was filtered a second time over a Celite pad. The filtrate was concentrated under pressure to provide (2R)-2-amino-5-methyl-hexane-1,5-diol (338.4 mg, 130%) as a yellow oil. The crude was used for the next step without purification. ¹H NMR (400 MHz, DMSO-d₆) δ 3.40-3.28 (m, 1H), 3.25-3.11 (m, 1H), 2.64 (br. s, 1H), 1.81 (s, 2H), 1.51-1.37 (m, 2H), 1.37-1.29 (m, 1H), 1.29-1.18 (m, 1H), 1.06 (d, J=1.0 Hz, 6H). ESI-MS m/z calc. 147.1259, found 148.4 (M+1)⁺; Retention time: 0.22 minutes, LC method X.

### Step 5: 3-[[4-[(2R)-2-Amino-5-hydroxy-5-methyl-hexoxy]-6-(2,6-dimethylphenyl)-5-methyl-pyrimidin-2-yl]sulfamoyl]benzoic acid

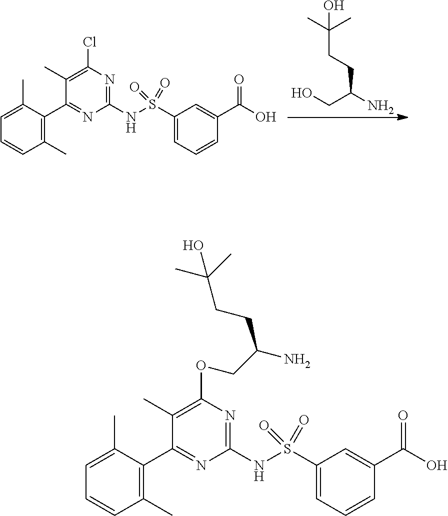

To a solution of 3-[[4-chloro-6-(2,6-dimethylphenyl)-5-methyl-pyrimidin-2-yl]sulfamoyl]benzoic acid (6.8 g, 15.745 mmol) and (2R)-2-amino-5-methyl-hexane-1,5-diol (2.8 g, 19.020 mmol) in tetrahydrofuran (55 mL) was slowly added sodium tert-butoxide in tetrahydrofuran (32 mL of 2 M, 64.000 mmol) and the mixture was stirred at room temperature for 2 hours. The reaction was partitioned between ethyl acetate (90 mL) and aqueous 1 N hydrochloric acid (30 mL). The aqueous phase was extracted with ethyl acetate (2×60 mL) and 2-methyltetrahydrofuran (4×100 mL). The combined organic layers were dried over sodium sulfate, filtered and concentrated to dryness. The residue was triturated with ethyl acetate (100 mL), the precipitate was filtered and washed with ethyl acetate (2×30 mL). The product was further dried under vacuum pump to afford 3-[[4-[(2R)-2-amino-5-hydroxy-5-methyl-hexoxy]-6-(2,6-dimethylphenyl)-5-methyl-pyrimidin-2-yl]sulfamoyl]benzoic acid (hydrochloride salt) (7 g, 71%) as a white solid. ¹H NMR (400 MHz, DMSO-d₆) δ 8.52-8.44 (m, 1H), 8.14-8.01 (m, 2H), 7.64 (t, J=7.7 Hz, 1H), 7.29-7.20 (m, 1H), 7.13 (d, J=7.6 Hz, 2H), 4.45-4.38 (m, 1H), 4.35-4.26 (m, 1H), 3.61-3.49 (m, 1H), 1.91 (d, J=6.4 Hz, 6H), 1.82-1.71 (m, 2H), 1.66 (s, 3H), 1.59-1.31 (m, 3H), 1.09 (d, J=5.1 Hz, 6H). ESI-MS m/z calc. 542.2199, found 543.2 (M+1)⁺; Retention time: 2.3 minutes, LC method Y.

### Step 6: 3-[[4-(2,6-Dimethylphenyl)-6-[(2R)-5-hydroxy-5-methyl-2-(spiro[2.3]hexan-5-ylamino)hexoxy]-5-methyl-pyrimidin-2-yl]sulfamoyl]benzoic acid

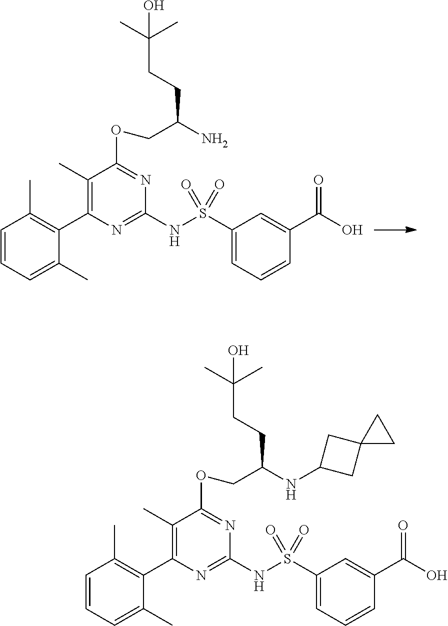 [SMILES: Cc1cccc(C)c1-c1nc(NS(=O)(=O)c2cccc(C(=O)O)c2)nc(OC[C@H](N)CCC(C)(C)O)c1C ; Cc1cccc(C)c1-c1nc(NS(=O)(=O)c2cccc(C(=O)O)c2)nc(OC[C@@H](CCC(C)(C)O)NC2CC3(CC3)C2)c1C]

A solution of 3-[[4-[(2R)-2-amino-5-hydroxy-5-methyl-hexoxy]-6-(2,6-dimethylphenyl)-5-methyl-pyrimidin-2-yl]sulfamoyl]benzoic acid (200 mg, 0.3686 mmol), spiro[2.3]hexan-5-one (212 mg, 2.2054 mmol) and Acetic acid (4 mg, 0.0038 mL, 0.0666 mmol) in acetonitrile (2 mL) and methanol (2 mL) were stirred for 1 h, then sodium cyanoborohydride (140 mg, 2.2278 mmol) was added then and left stirring for 1 h then More spiro[2.3]hexan-5-one (212 mg, 2.2054 mmol) was added to the reaction mixture which was then stirred 30 min. then sodium cyanoborohydride (140 mg, 2.2278 mmol) was added and the reaction was left stirring at room temperature for 4 h. The reaction was finished. The reaction mixture was diluted with saturated aqueous ammonium chloride (1 mL), then partitioned between water (20 mL) and ethyl acetate (50 mL). The aqueous mixture was separated and washed with ethyl acetate (2×50 mL). The organic fractions were combined, dried with sodium sulfate and concentrated under reduced pressure. The resulting residue was purified on silica gel using 0 then 5% methanol in dichloromethane. The resulting material was purified by reverse phase chromatography on a Cis support using 0 to 60% methanol in water gradient to provide 3-[[4-(2,6-dimethylphenyl)-6-[(2R)-5-hydroxy-5-methyl-2-(spiro[2.3]hexan-5-ylamino)hexoxy]-5-methyl-pyrimidin-2-yl]sulfamoyl]benzoic acid (56 mg, 24%) as a white powder ¹H NMR (400 MHz, DMSO-d₆) δ 8.54 (br. s., 1H), 8.08 (d, J=6.8 Hz, 1H), 7.97 (d, J=7.8 Hz, 1H), 7.67-7.54 (m, 1H), 7.24 (br. s., 1H), 7.13 (d, J=6.8 Hz, 2H), 4.22 (br. s., 2H), 3.68 (br. s., 1H), 3.06 (br. s., 1H), 2.28-1.85 (m, 11H), 1.61 (br. s., 5H), 1.40 (d, J=8.8 Hz, 3H), 1.06 (s, 6H), 0.48-0.30 (m, 4H). ESI-MS m/z calc. 622.2825, found 623.2 (M+1)⁺; Retention time: 1.37 minutes (LC method X).

### Step 7: (11R)-6-(2,6-dimethylphenyl)-11-(3-hydroxy-3-methyl-butyl)-7-methyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 106)

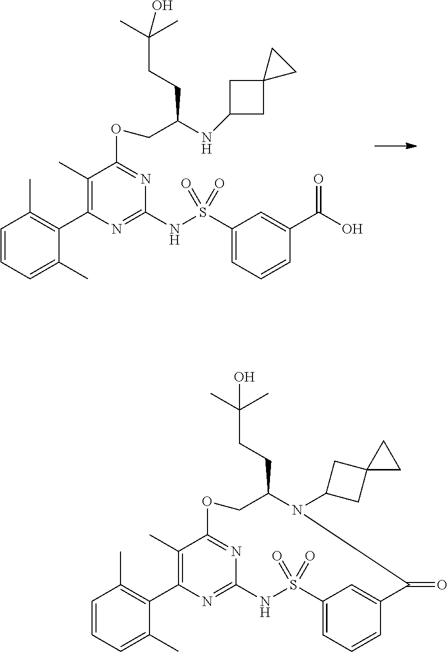 [SMILES: Cc1cccc(C)c1-c1nc(NS(=O)(=O)c2cccc(C(=O)O)c2)nc(OC[C@@H](CCC(C)(C)O)NC2CC3(CC3)C2)c1C ; Cc1cccc(C)c1-c1nc2nc(c1C)OC[C@@H](CCC(C)(C)O)N(C1CC3(CC3)C1)C(=O)c1cccc(c1)S(=O)(=O)N2]

3-[[4-(2,6-Dimethylphenyl)-6-[(2R)-5-hydroxy-5-methyl-2-(spiro[2.3]hexan-5-ylamino)hexoxy]-5-methyl-pyrimidin-2-yl]sulfamoyl]benzoic acid (56 mg, 0.1032 mmol) and triethylamine (50.820 mg, 70 μL, 0.5022 mmol) were dissolved in DMF (0.5 mL) and ethylacetate (1.5 mL), T3P (50% in ethyl acetate) (106.90 mg, 200 μL, 0.1680 mmol) was added to that solution maintained at 0° C. The reaction mixture was then left stirring at room temperature for 1 h. LCMS showed complex mixture with traces of desired product forming. More triethylamine (72.600 mg, 100 μL, 0.7175 mmol) and T3P (50% in ethyl acetate) (213.80 mg, 400 μL, 0.3360 mmol) were added to the solution and the reaction mixture was left stirring at room temperature overnight. The reaction mixture was concentrated under reduced pressure and the resulting residue was left under high vacuum for 1 h then purified by reverse phase chromatography using a gradient of 0.5 to 100% acetonitrile in water. The pure fractions were combined and concentrated on the freeze-drier to provide (11R)-6-(2,6-dimethylphenyl)-11-(3-hydroxy-3-methyl-butyl)-7-methyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (20 mg, 31%) as a white fluffy solid ¹H NMR (400 MHz, DMSO-d₆, 80° C.) δ 8.44 (s, 1H), 7.89 (d, J=6.6 Hz, 1H), 7.74-7.59 (m, 2H), 7.32-7.20 (m, 1H), 7.15 (d, J=7.6 Hz, 1H), 7.11 (d, J=7.8 Hz, 1H), 5.20 (dd, J=10.8, 4.4 Hz, 1H), 4.39 (t, J=11.0 Hz, 1H), 4.24 (quin, J=8.4 Hz, 1H), 3.86-3.56 (m, 2H), 3.28-3.17 (m, 2H), 2.23-2.12 (m, 2H), 2.04 (s, 3H), 1.86 (s, 3H), 1.73-1.57 (m, 5H), 1.29 (ddd, J=13.3, 10.5, 5.6 Hz, 1H), 0.91 (s, 3H), 0.90 (s, 3H), 0.87-0.80 (m, 1H), 0.56-0.44 (m, 4H). ESI-MS m/z calc. 604.2719, found 605.3 (M+1)⁺; Retention time: 4.12 minutes, LC method Y.

### Example 36: Preparation of Compound 107

### Step 1: (11R)-6-(2,6-Dimethylphenyl)-11-(3-hydroxy-3-methyl-butyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 107)

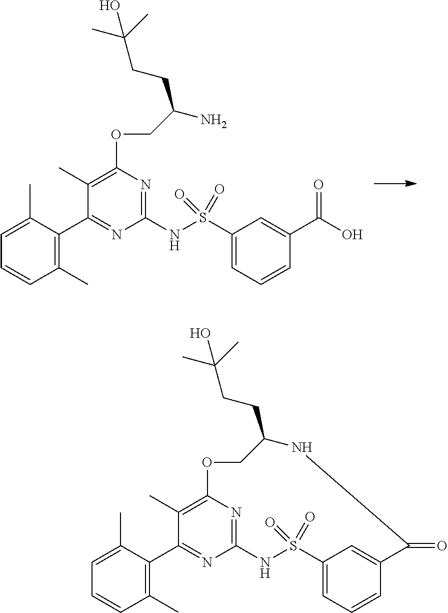 [SMILES: Cc1cccc(C)c1-c1nc(NS(=O)(=O)c2cccc(C(=O)O)c2)nc(OC[C@H](N)CCC(C)(C)O)c1C ; Cc1cccc(C)c1-c1nc2nc(c1C)OC[C@@H](CCC(C)(C)O)NC(=O)c1cccc(c1)S(=O)(=O)N2]

To a solution of 3-[[4-[(2R)-2-amino-5-hydroxy-5-methyl-hexoxy]-6-(2,6-dimethylphenyl)-5-methyl-pyrimidin-2-yl]sulfamoyl]benzoic acid (hydrochloride salt) (100 mg, 0.1727 mmol) in DMF (1.5 mL) and EtOAc (7 mL) was added TEA (145.20 mg, 0.2 mL, 1.4349 mmol). The solution was cooled down to 0° C. and Propylphosphonic anhydride solution (0.5 mL of 50% w/v, 0.7857 mmol) was added. The reaction was stirred for 16 h at room temperature and then ethyl acetate was removed under vacuo. The resulting solution was directly purified by reverse phase chromatography with a C₁₈ 50 g cartridge using a gradient of MeCN in water (5 to 100%). It afforded after lyophilization (11R)-6-(2,6-dimethylphenyl)-11-(3-hydroxy-3-methyl-butyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (50 mg, 55%) as a white solid. ¹H NMR (400 MHz, DMSO-d₆) δ 12.93 (br. s., 1H), 8.49 (s, 1H), 7.89 (br. s., 1H), 7.82 (d, J=9.0 Hz, 1H), 7.65 (br. s., 2H), 7.27 (br. s., 1H), 7.16 (d, J=7.3 Hz, 1H), 7.12 (d, J=6.8 Hz, 1H), 5.18 (d, J=9.0 Hz, 1H), 4.09-3.87 (m, 2H), 3.29-3.20 (m, 1H), 2.03 (br. s., 3H), 1.88 (br. s., 3H), 1.68-1.53 (m, 4H), 1.53-1.34 (m, 2H), 0.99-0.77 (m, 7H). ESI-MS m/z calc. 524.2093, found 525.3 (M+1)⁺; Retention time: 3.2 minutes, LC method Y.

### Example 37: Preparation of Compound 108

### Step 1: 3-[[4-(2,6-Dimethylphenyl)-6-[(2R)-4-hydroxy-4-methyl-2-(spiro[2.3]hexan-5-ylamino)pentoxy]-5-methyl-pyrimidin-2-yl]sulfamoyl]benzoic acid

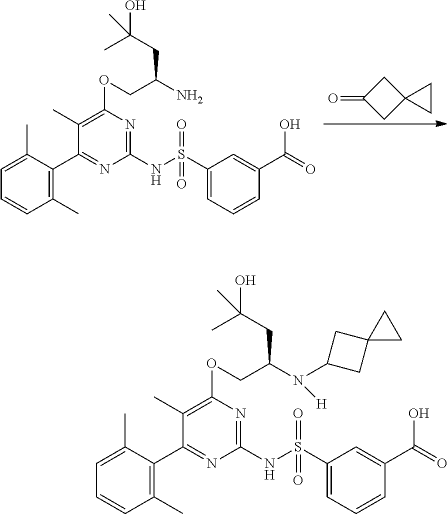 [SMILES: Cc1cccc(C)c1-c1nc(NS(=O)(=O)c2cccc(C(=O)O)c2)nc(OC[C@H](N)CC(C)(C)O)c1C ; O=C1CC2(CC2)C1 ; Cc1cccc(C)c1-c1nc(NS(=O)(=O)c2cccc(C(=O)O)c2)nc(OC[C@@H](CC(C)(C)O)NC2CC3(CC3)C2)c1C]

3-[[4-[(2R-2-Amino-4-hydroxy-4-methyl-pentoxy]-6-(2,6-dimethylphenyl-5-methyl-pyrimidin-2-yl]sulfamoyl]benzoic acid (hydrochloride salt) (50 mg, 0.0858 mmol), spiro[2.3]hexan-5-one (45 mg, 0.4681 mmol) and acetic acid (16.896 mg, 16 μL, 0.2814 mmol) were stirred at room temperature for 1 hour in acetonitrile (1 mL) and methanol (1 mL). Sodium cyanoborohydride (29 mg, 0.4615 mmol) was added and the reaction mixture was stirred for 24 hours. The mixture was combined with another reaction run on 200 mg of 3-[[4-[(2R)-2-Amino-4-hydroxy-4-methyl-pentoxy]-6-(2,6-dimethylphenyl)-5-methyl-pyrimidin-2-yl]sulfamoyl]benzoic acid (hydrochloride salt) starting material. The crude mixture was partitioned between ethyl acetate (25 mL) and brine (30 mL). The aqueous phase was separated and extracted with ethyl acetate (2×25 mL). The combined organic phases were washed with brine (30 mL), dried over sodium sulfate, filtered and concentrated in vacuo. The product was purified by normal phase chromatography (24+10 g silica gel, eluting 0 to 15% methanol in dichloromethane) to give 3-[[4-(2,6-dimethylphenyl)-6-[(2R)-4-hydroxy-4-methyl-2-(spiro[2.3]hexan-5-ylamino)pentoxy]-5-methyl-pyrimidin-2-yl]sulfamoyl]benzoic acid (194 mg, 72 combined yield %) as a white solid. ¹H NMR (400 MHz, DMSO-d₆) δ 8.50 (s, 1H), 8.11 (d, J=7.8 Hz, 1H), 8.04 (d, J=7.6 Hz, 1H), 7.63 (t, J=7.8 Hz, 1H), 7.30-7.22 (m, 1H), 7.14 (d, J=7.6 Hz, 2H), 4.21 (d, J=3.7 Hz, 2H), 3.85-3.74 (m, 1H), 3.42-3.32 (m, 1H), 2.31-2.21 (m, 1H), 2.20-2.02 (m, 3H), 1.92 (d, J=5.1 Hz, 6H), 1.73-1.58 (m, 5H), 1.31-1.09 (m, 7H), 0.48-0.40 (m, 2H), 0.40-0.29 (m, 2H). ESI-MS m/z calc. 608.2669, found 609.2 (M+1)⁺; Retention time: 1.38 minutes (LC method X).

### Step 2: (11R)-6-(2,6-Dimethylphenyl)-11-(2-hydroxy-2-methyl-propyl)-7-methyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 108)

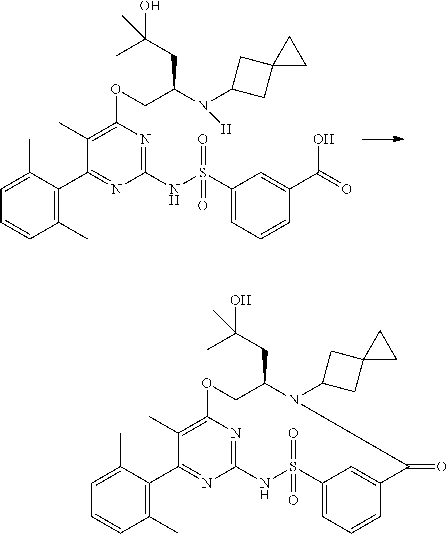 [SMILES: Cc1cccc(C)c1-c1nc(NS(=O)(=O)c2cccc(C(=O)O)c2)nc(OC[C@@H](CC(C)(C)O)NC2CC3(CC3)C2)c1C ; Cc1cccc(C)c1-c1nc2nc(c1C)OC[C@@H](CC(C)(C)O)N(C1CC3(CC3)C1)C(=O)c1cccc(c1)S(=O)(=O)N2]

To a solution of 3-[[4-(2,6-dimethylphenyl)-6-[(2R)-4-hydroxy-4-methyl-2-(spiro[2.3]hexan-5-ylamino)pentoxy]-5-methyl-pyrimidin-2-yl]sulfamoyl]benzoic acid (187 mg, 0.2980 mmol) in ethyl acetate (14 mL) and DMF (2.2 mL) was added triethylamine (181.50 mg, 250 μL, 1.7937 mmol) and propylphosphonic anhydride solution in ethyl acetate (570 μL of 50% w/v, 0.8957 mmol). The reaction was stirred at room temperature for 2 h. Triethylamine (181.50 mg, 250 μL, 1.7937 mmol) and propylphosphonic anhydride solution in ethyl acetate (570 μL of 50% w/v, 0.8957 mmol) were added and the reaction mixture was stirred at room temperature for 2. The ethyl acetate was removed under reduced pressure. The residue was purified by reverse phase chromatography on C₁₈ (50 g, eluting 20 to 100% acetonitrile in water) and freeze-dried to give (11R)-6-(2,6-dimethylphenyl)-11-(2-hydroxy-2-methyl-propyl)-7-methyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (30 mg, 16%) as a white fluffy solid. ¹H NMR (400 MHz, DMSO-d₆, (taken at 80° C.) δ 8.46 (s, 1H), 7.89 (d, J=6.8 Hz, 1H), 7.67-7.59 (m, 2H), 7.27-7.21 (m, 1H), 7.15 (d, J=7.3 Hz, 1H), 7.10 (d, J=7.6 Hz, 1H), 5.17 (dd, J=10.5, 4.4 Hz, 1H), 4.37-4.21 (m, 2H), 3.92-3.83 (m, 1H), 3.77 (br. s., 1H), 3.26 (t, J=9.3 Hz, 1H), 3.16 (t, J=9.7 Hz, 1H), 2.25-2.14 (m, 2H), 2.05 (s, 3H), 1.85 (dd, J=14.9, 8.3 Hz, 1H), 1.80 (s, 3H), 1.65-1.56 (m, 4H), 0.75 (s, 3H), 0.67 (s, 3H), 0.57-0.43 (m, 4H). ESI-MS m/z calc. 590.2563, found 591.2 (M+1)⁺; Retention time: 4.22 minutes, LC method Y.

### Example 38: Characterization of Compounds 109-115

The compounds in the following tables were prepared in a manner analogous to that described above using commercially available reagents and intermediates described herein.

TABLE 9

| Compound | | LCMS Rt | Calc. | | LCMS |
|---|---|---|---|---|---|
| number | Structure | (min) | mass | M + 1 | Method |
| 109 | 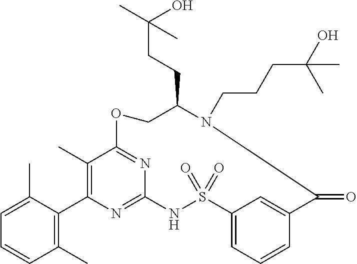 [SMILES: Cc1cccc(C)c1-c1nc2nc(c1C)OC[C@@H](CCC(C)(C)O)N(CCCC(C)(C)O)C(=O)c1cccc(c1)S(=O)(=O)N2] | 3.42 | 624.298 | 625.3 | S |
| 110 | 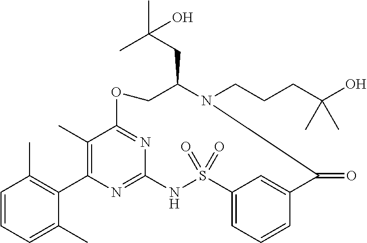 [SMILES: Cc1cccc(C)c1-c1nc2nc(c1C)OC[C@@H](CC(C)(C)O)N(CCCC(C)(C)O)C(=O)c1cccc(c1)S(=O)(=O)N2] | 3.44 | 610.283 | 611.3 | S |
| 111 | 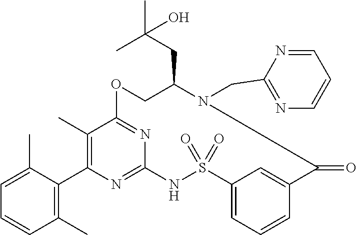 [SMILES: Cc1cccc(C)c1-c1nc2nc(c1C)OC[C@@H](CC(C)(C)O)N(Cc1ncccn1)C(=O)c1cccc(c1)S(=O)(=O)N2] | 3.39 | 602.231 | 603.3 | S |
| 112 | 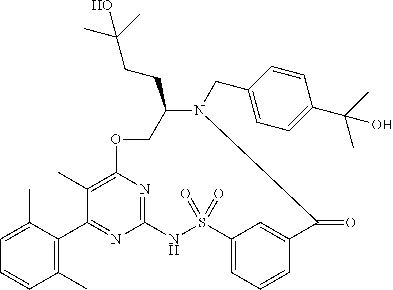 [SMILES: Cc1cccc(C)c1-c1nc2nc(c1C)OC[C@@H](CCC(C)(C)O)N(Cc1ccc(C(C)(C)O)cc1)C(=O)c1cccc(c1)S(=O)(=O)N2] | 3.65 | 672.298 | 655.3 (M + 1 − H₂O) | S |
| 113 | 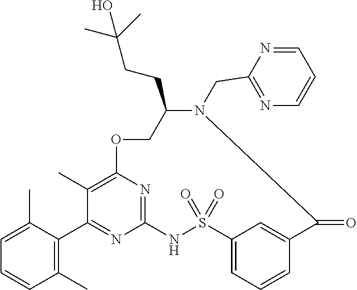 [SMILES: Cc1cccc(C)c1-c1nc2nc(c1C)OC[C@@H](CCC(C)(C)O)N(Cc1ncccn1)C(=O)c1cccc(c1)S(=O)(=O)N2] | 3.37 | 616.247 | 617.3 | S |
| 114 | 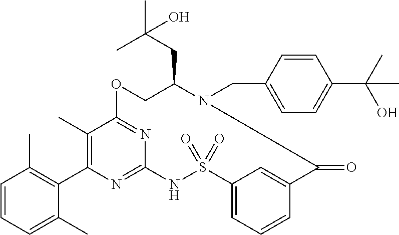 [SMILES: Cc1cccc(C)c1-c1nc2nc(c1C)OC[C@@H](CC(C)(C)O)N(Cc1ccc(C(C)(C)O)cc1)C(=O)c1cccc(c1)S(=O)(=O)N2] | 3.69 | 658.283 | 659.3 | S |
| 115 | 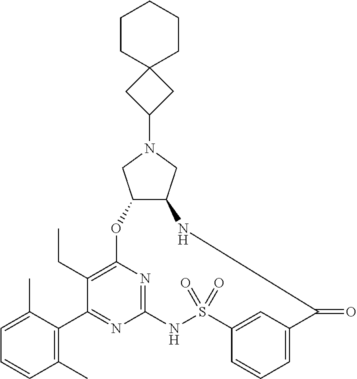 [SMILES: CCc1c2nc(nc1-c1c(C)cccc1C)NS(=O)(=O)c1cccc(c1)C(=O)N[C@@H]1CN(C3CC4(CCCCC4)C3)C[C@H]1O2] | 1.44 | 615.288 | 616.2 | A |

TABLE 10

| Compound | |
|---|---|
| number | NMR |
| 109 | ¹H NMR (400 MHz, DMSO-d₆, 80° C.) δ 8.43 (s, 1H), 7.93-7.86 (m, 1H), 7.69- |
| | 7.60 (m, 2H), 7.25 (t, J = 7.6 Hz, 1H), 7.15 (d, J = 7.6 Hz, 1H), 7.11 (d, J = |
| | 7.6 Hz, 1H), 5.18 (dd, J = 10.6, 4.3 Hz, 1H), 4.39-4.28 (m, 1H), 3.90 (s, 1H), |
| | 3.83-3.72 (m, 2H), 3.66-3.56 (m, 1H), 3.17-3.09 (m, 1H), 2.05-2.03 (m, |
| | 3H), 1.87 (s, 3H), 1.83-1.74 (m, 1H), 1.74-1.55 (m, 6H), 1.53-1.40 (m, 2H), |
| | 1.27-1.18 (m, 1H), 1.17-1.13 (m, 6H), 0.93-0.87 (m, 6H), 0.86-0.78 (m, 1H). |
| 110 | ¹H NMR (400 MHz, DMSO-d₆, 80° C.) δ 11.99 (br. s., 1H), 8.46 (s, 1H), 7.89 |
| | (t, J = 3.7 Hz, 1H), 7.62 (d, J = 4.6 Hz, 2H), 7.24 (t, J = 7.6 Hz, 1H), 7.15 (d, |
| | J = 7.6 Hz, 1H), 7.10 (d, J = 7.6 Hz, 1H), 5.15 (dd, J = 10.4, 4.3 Hz, 1H), 4.25 |
| | (t, J = 11.1 Hz, 1H), 4.08-3.98 (m, 1H), 3.88 (br. s., 1H), 3.76 (s, 1H), 3.69- |
| | 3.58 (m, 1H), 3.12-3.05 (m, 1H), 2.05 (s, 3H), 1.90-1.78 (m, 5H), 1.70-1.58 |
| | (m, 4H), 1.56-1.41 (m, 3H), 1.15 (s, 6H), 0.78 (s, 3H), 0.64 (s, 3H). |
| 111 | ¹H NMR (400 MHz, DMSO-d₆, 80° C.) δ 8.79 (d, J = 4.9 Hz, 2H), 8.71 (s, 1H), |
| | 7.91 (d, J = 7.6 Hz, 1H), 7.66-7.57 (m, 2H), 7.39 (t, J = 4.9 Hz, 1H), 7.29- |
| | 7.22 (m, 1H), 7.15 (d, J = 7.6 Hz, 1H), 7.11 (d, J = 7.6 Hz, 1H), 5.47-5.36 (m, |
| | 1H), 5.00 (d, J = 17.1 Hz, 1H), 4.68 (d, J = 16.9 Hz, 1H), 4.26 (br. s., 1H), 4.23- |
| | 4.14 (m, 2H), 2.05 (s, 3H), 1.96 (dd, J = 15.4, 7.1 Hz, 1H), 1.82 (s, 3H), 1.64- |
| | 1.56 (m, 4H), 0.91 (s, 3H), 0.61 (s, 3H). |
| 112 | ¹H NMR (400 MHz, DMSO-d₆) δ 8.49 (s, 1H), 7.96-7.87 (m, 1H), 7.75-7.65 |
| | (m, 2H), 7.53-7.45 (m, 2H), 7.39-7.37 (m, 2H), 7.29-7.21 (m, 1H), 7.15- |
| | 7.10 (m, 2H), 5.10-4.97 (m, 2H), 4.69 (s, 1H), 4.33 (d, J = 15.4 Hz, 1H), 4.17- |
| | 4.06 (m, 1H), 3.93-3.87 (m, 1H), 3.76 (s, 1H), 2.02 (s, 3H), 1.87 (s, 3H), 1.83- |
| | 1.74 (m, 1H), 1.70-1.63 (m, 1H), 1.60 (s, 3H), 1.48 (m, 6H), 1.29-1.17 (m, |
| | 1H), 0.90 (d, J = 5.1 Hz, 6H), 0.88-0.81 (m, 1H). |
| 113 | ¹H NMR (400 MHz, DMSO-d₆) δ 12.92 (br. s., 1H), 8.81 (d, J = 4.9 Hz, 2H), |
| | 8.66 (br. s., 1H), 7.89 (br. s., 1H), 7.65 (br. s., 2H), 7.40 (t, J = 4.9 Hz, 1H), |
| | 7.29-7.26 (m, 1H), 7.18-7.12 (m, 2H), 5.45-5.43 (br. s., 1H), 4.95 (d, J = |
| | 16.9 Hz, 1H), 4.69 (d, J = 16.6 Hz, 1H), 4.27 (t, J = 11.0 Hz, 1H), 4.09 (br. s., |
| | 1H), 3.94-3.89 (m, 1H), 2.03 (br. s., 3H), 1.91 (br. s., 3H), 1.85-1.74 (m, 1H), |
| | 1.69-1.52 (m, 4H), 1.37 1.26 (m, 1H), 0.95 (d, |
| | J = 9.0 Hz, 6H), 0.88-0.86 (m, 1H). |
| 114 | ¹H NMR (400 MHz, DMSO-d₆, 80° C.) δ 12.16 (br. s., 1H), 8.52 (s, 1H), 7.96- |
| | 7.89 (m, 1H), 7.73-7.62 (m, 2H), 7.50-7.46 (m, 2H), 7.37 (d, J = 8.3 Hz, 2H), |
| | 7.29-7.21 (m, 1H), 7.14 (d, J = 7.6 Hz, 1H), 7.10 (d, J = 7.6 Hz, 1H), 5.10- |
| | 4.99 (m, 2H), 4.68 (s, 1H), 4.32 (d, J = 15.4 Hz, 1H), 4.25-4.16 (m, 1H), 4.13- |
| | 4.04 (m, 1H), 3.83 (s, 1H), 2.08-1.98 (m, 4H), 1.82 (s, 3H), 1.61 (s, 3H), 1.55 |
| | (d, J = 13.7 Hz, 1H), 1.46 (s, 6H), 0.86 (s, 3H), 0.63 (s, 3H). |

### Example 39: Preparation of Compound 116

### Step 1: 3-[[4-1(2R)-2-Aminopropoxyl-6-(2,6-dimethylphenyl)-5-methyl-pyrimidin-2-yl]sulfamoyl]benzoic acid

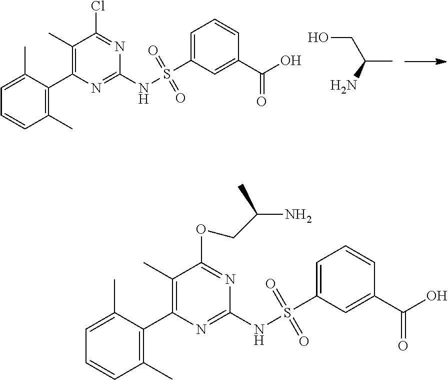

3-[[4-chloro-6-(2,6-dimethylphenyl)-5-methyl-pyrimidin-2-yl]sulfamoyl]benzoic acid (743.6 mg, 1.722 mmol) and (2R)-2-aminopropan-1-ol (160 mg, 2.130 mmol) were combined in THF (3 mL) and sodium tert-butoxide (726 mg, 7.554 mmol) was added. The reaction was stirred at room temperature for 10 min. The reaction was made acidic by the addition of 1M HCl and extracted with a mixture of ethyl acetate (10 mL) and methanol (5 mL) two times. The organics were combined, washed with brine, dried over sodium sulfate and evaporated. The crude material was triturated with diethylether and the white solid was collected and dried to give 3-[[4-[(2R)-2-aminopropoxy]-6-(2,6-dimethylphenyl)-5-methyl-pyrimidin-2-yl]sulfamoyl]benzoic acid (hydrochloride salt) (485 mg, 560%) as a white solid. ESI-MS m/z calc. 470.16238, found 471.1 (M+1)⁺; Retention time: 0.39 minutes. LC method D.

### Step 2: 3-({4-[(2R)-2-[(6-{[(tert-Butoxy)carbonyl]amino}spiro[3.3]heptan-2-yl)amino]propoxy]-6-(2,6-dimethylphenyl)-5-methylpyrimidin-2-yl}sulfamoyl)benzoic acid

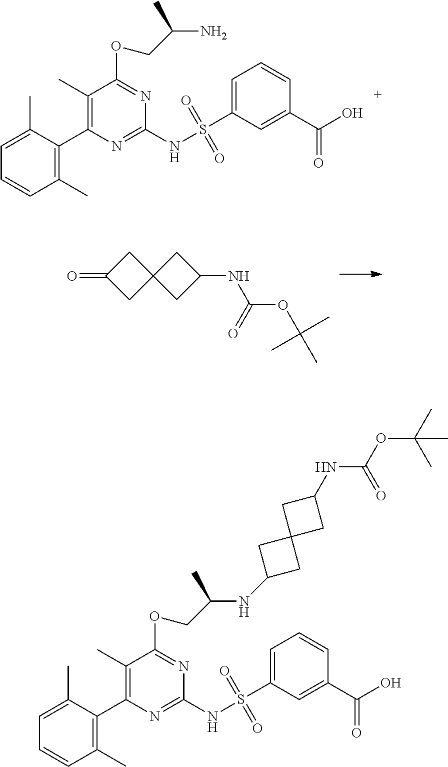 [SMILES: Cc1cccc(C)c1-c1nc(NS(=O)(=O)c2cccc(C(=O)O)c2)nc(OC[C@@H](C)N)c1C ; CC(C)(C)OC(=O)NC1CC2(CC(=O)C2)C1 ; Cc1cccc(C)c1-c1nc(NS(=O)(=O)c2cccc(C(=O)O)c2)nc(OC[C@@H](C)NC2CC3(CC(NC(=O)OC(C)(C)C)C3)C2)c1C]

3-[[4-[(2R)-2-Aminopropoxy]-6-(2,6-dimethylphenyl)-5-methyl-pyrimidin-2-yl]sulfamoyl]benzoic acid (hydrochloride salt) (100 mg, 0.1972 mmol), tert-butyl N-(2-oxospiro[3.3]heptan-6-yl)carbamate (approximately 66.64 mg, 0.2958 mmol), and sodium triacetoxyborohydride (approximately 209.0 mg, 0.9860 mmol) were combined in DCE (0.3 mL) and stirred at room temperature for 2 h. The reaction mixture was partitioned between ethyl acetate and a 1 M HCl solution. The organics were separated, washed with brine, dried over sodium sulfate and evaporated. The crude material was purified by reverse phase HPLC utilizing a gradient of 1-99% acetonitrile in 5 mM aqueous HCl to yield 3-({4-[(2R)-2-[(6-{[(tert-butoxy)carbonyl]amino}spiro[3.3]heptan-2-yl)amino]propoxy]-6-(2,6-dimethylphenyl)-5-methylpyrimidin-2-yl}sulfamoyl)benzoic acid (hydrochloride salt) (56 mg, 40%). ESI-MS m/z calc. 679.30396, found 680.8 (M+1)⁺; Retention time: 0.53 minutes; LC method D.

### Step 3: tert-Butyl N-{6-[(11R)-6-(2,6-dimethylphenyl)-7,11-dimethyl-2,2,13-trioxo-9-oxa-2λ⁶-thia-3,5,12,19-tetraazatricyclo[12.3.1.14,8]nonadeca-1(18),4,6,8(19),14,16-hexaen-12-yl]spiro[3.3]heptan-2-yl}carbamate (Compound 116)

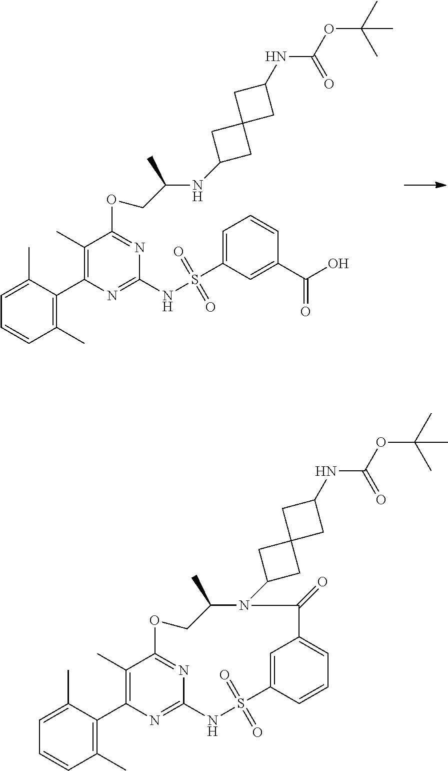 [SMILES: Cc1cccc(C)c1-c1nc(NS(=O)(=O)c2cccc(C(=O)O)c2)nc(OC[C@@H](C)NC2CC3(CC(NC(=O)OC(C)(C)C)C3)C2)c1C ; Cc1cccc(C)c1-c1nc2nc(c1C)OC[C@@H](C)N(C1CC3(CC(NC(=O)OC(C)(C)C)C3)C1)C(=O)c1cccc(c1)S(=O)(=O)N2]

3-({4-[(2R)-2-[(6-{[(tert-Butoxy)carbonyl]amino}spiro[3.3]heptan-2-yl)amino]propoxy]-6-(2,6-dimethylphenyl)-5-methylpyrimidin-2-yl}sulfamoyl)benzoic acid (hydrochloride salt) (19.1 mg, 0.02667 mmol), HATU (11.3 mg, 0.02972 mmol), and triethylamine (19 μL, 0.1363 mmol) were combined in DMF (1 mL) and the reaction was stirred at room temperature for 1 h. The reaction mixture was filtered and purified by reverse phase HPLC utilizing a gradient of 1-99% acetonitrile in 5 mM aqueous HCl to yield tert-butyl N-{6-[(11R)-6-(2,6-dimethylphenyl)-7,11-dimethyl-2,2,13-trioxo-9-oxa-2λ⁶-thia-3,5,12,19-tetraazatricyclo[12.3.1.14,8]nonadeca-1(18),4,6,8(19),14,16-hexaen-12-yl]spiro[3.3]heptan-2-yl}carbamate (10.1 mg, 57%) as a white solid. ESI-MS m/z calc. 661.2934, found 662.9 (M+1)⁺; Retention time: 1.88 minutes, LC method A.

### Example 40: Preparation of Compound 117

### Step 1: (11R)-6-(2,6-Dimethylphenyl)-7,11-dimethyl-9-oxa-2λ⁶-thia-3,5,12,19-tetraazatricyclo[12.3.1.14,8]nonadeca-1(18),4,6,8(19),14,16-hexaene-2,2,13-trione (Compound 117)

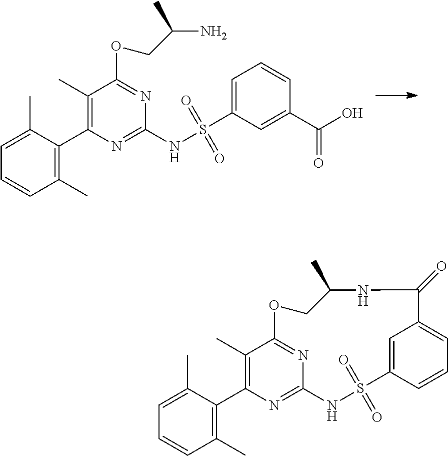 [SMILES: Cc1cccc(C)c1-c1nc(NS(=O)(=O)c2cccc(C(=O)O)c2)nc(OC[C@@H](C)N)c1C ; Cc1cccc(C)c1-c1nc2nc(c1C)OC[C@@H](C)NC(=O)c1cccc(c1)S(=O)(=O)N2]

3-[[4-[(2R)-2-Aminopropoxy]-6-(2,6-dimethylphenyl)-5-methyl-pyrimidin-2-yl]sulfamoyl]benzoic acid (hydrochloride salt) (25 mg, 0.04931 mmol) and HATU (19.7 mg, 0.05181 mmol) were dissolved in DMF (1 mL) and DIEA (47 μL, 0.2698 mmol) was added. The reaction mixture was stirred at room temperature for 1 h. The mixture was filtered and purified by reverse phase HPLC utilizing a gradient of 1-99% acetonitrile in 5 mM aqueous HCl to yield (11R)-6-(2,6-dimethylphenyl)-7,11-dimethyl-9-oxa-2λ⁶-thia-3,5,12,19-tetraazatricyclo[12.3.1.14,8]nonadeca-1(18),4,6,8(19),14,16-hexaene-2,2,13-trione (4.3 mg, 18%) as a white solid. ESI-MS m/z calc. 452.15182, found 453.1 (M+1)⁺; Retention time: 1.26 minutes, LC method A.

### Example 41: Preparation of Compound 118

### Step 1: Methyl 6-[[4-chloro-6-(2,6-dimethylphenyl)-5-methyl-pyrimidin-2-yl]sulfamoyl]pyridine-2-carboxylate

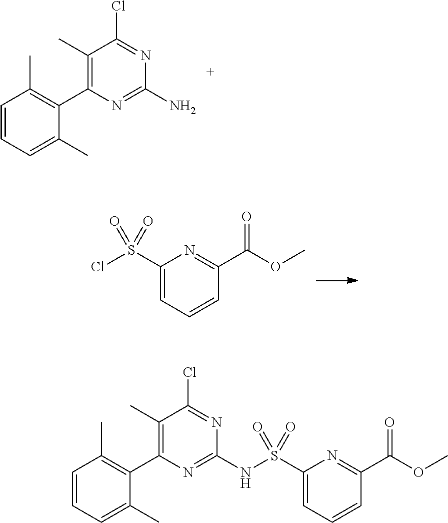 [SMILES: Cc1cccc(C)c1-c1nc(N)nc(Cl)c1C ; COC(=O)c1cccc(S(=O)(=O)Cl)n1 ; COC(=O)c1cccc(S(=O)(=O)Nc2nc(Cl)c(C)c(-c3c(C)cccc3C)n2)n1]

Into a solution of 4-chloro-6-(2,6-dimethylphenyl)-5-methyl-pyrimidin-2-amine (6.56 g, 26.481 mmol) and methyl 6-chlorosulfonylpyridine-2-carboxylate (8.12 g, 34.459 mmol) in anhydrous THF (100 mL) was added a solution of LiHMDS (53 mL of 1 M, 53.000 mmol) in THE at −78° C. dropwise. After the complete addition, the reaction temperature was raised to 0° C. gradually. The reaction was then quenched with 1 N HCl (aqueous) (50 mL). Two layers were separated, and the aqueous layer was extracted with ethyl acetate (2×100 mL). The combined organic layers were washed with brine (2×50 mL), dried over anhydrous sodium sulfate and concentrated under vacuum. The residue was purified by silica gel chromatography using 0 to 50% acetone in hexane to furnish methyl 6-[[4-chloro-6-(2,6-dimethylphenyl)-5-methyl-pyrimidin-2-yl]sulfamoyl]pyridine-2-carboxylate (9.063 g, 69%) as a yellow solid. ESI-MS m/z calc. 446.08154, found 447.0 (M+1)⁺; Retention time: 5.94 minutes (LC method S).

### Step 2: 6-[[4-Chloro-6-(2,6-dimethylphenyl)-5-methyl-pyrimidin-2-yl]sulfamoyl]pyridine-2-carboxylic acid

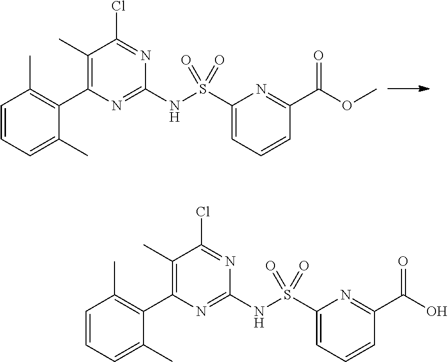 [SMILES: COC(=O)c1cccc(S(=O)(=O)Nc2nc(Cl)c(C)c(-c3c(C)cccc3C)n2)n1 ; Cc1cccc(C)c1-c1nc(NS(=O)(=O)c2cccc(C(=O)O)n2)nc(Cl)c1C]

Into a solution of methyl 6-[[4-chloro-6-(2,6-dimethylphenyl)-5-methyl-pyrimidin-2-yl]sulfamoyl]pyridine-2-carboxylate (9.063 g, 18.251 mmol) in THF (90 mL) was added an aqueous solution of sodium hydroxide (90 mL of 1 M, 90.000 mmol). The reaction mixture was stirred at room temperature for 1 hour. The volatiles were removed under vacuum. The aqueous solution was neutralized to pH 4-5 with 1 N HCl. The solid was collected by filtration, and then it was further purified by trituration with ethanol at elevated temperature and dried under vacuum. The product was found to contains trace amount of ethanol even after prolong time under vacuum. The product was then dissolved in acetonitrile and water, and lyophilized to furnish 6-[[4-chloro-6-(2,6-dimethylphenyl)-5-methyl-pyrimidin-2-yl]sulfamoyl]pyridine-2-carboxylic acid (6.0222 g, 72%) as a white powder. ESI-MS m/z calc. 432.0659, found 433.0 (M+1)⁺; Retention time: 2.25 minutes (LC method W), ¹H NMR (500 MHz, DMSO-d₆) δ 8.08-8.01 (m, 2H), 7.94 (d, J=8.2 Hz, 1H), 7.18 (t, J=7.6 Hz, 1H), 7.06 (d, J=7.6 Hz, 2H), 1.74 (d, J=4.9 Hz, 9H).

### Step 3: 6-[[4-(2,6-Dimethylphenyl)-5-methyl-6-[(2R)-4-methyl-2-(spiro[2.3]hexan-5-ylamino)pentoxy]pyrimidin-2-yl]sulfamoyl]pyridine-2-carboxylic acid

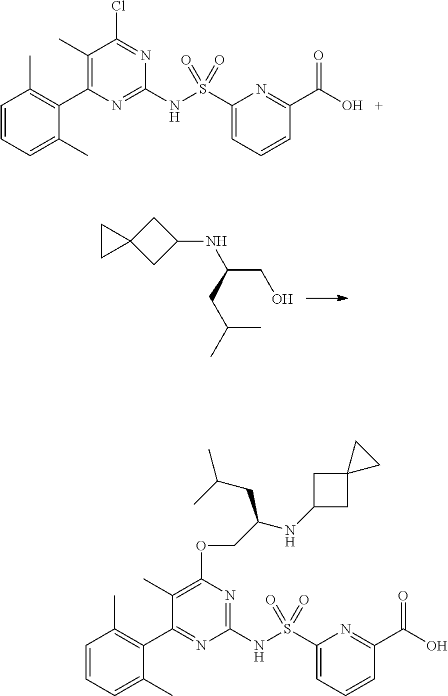 [SMILES: Cc1cccc(C)c1-c1nc(NS(=O)(=O)c2cccc(C(=O)O)n2)nc(Cl)c1C ; CC(C)C[C@H](CO)NC1CC2(CC2)C1 ; Cc1cccc(C)c1-c1nc(NS(=O)(=O)c2cccc(C(=O)O)n2)nc(OC[C@@H](CC(C)C)NC2CC3(CC3)C2)c1C]

In a 100 mL flask, 6-[[4-chloro-6-(2,6-dimethylphenyl)-5-methyl-pyrimidin-2-yl]sulfamoyl]pyridine-2-carboxylic acid (209 mg, 0.4828 mmol) and (2R)-4-methyl-2-(spiro[2.3]hexan-5-ylamino)pentan-1-ol (hydrochloride salt) (117 mg, 0.5005 mmol) were charged under nitrogen with anhydrous THF (3 mL) (suspension). Sodium tert-butoxide (203 mg, 2.112 mmol) was added. The suspension was stirred at room temperature for 2 hours The mixture was partitioned between ethyl acetate (30 mL) and aqueous 1M HCl (30 mL) and brine (20 mL). After separation, the aqueous phase was further extracted with EtOAc (3×30 mL). The combined extracts were dried over sodium sulfate and the solvents evaporated to give a crude material. The material was dissolved in DMSO (4 mL). The solution was microfiltered through a syringe filter disc and purified by reverse phase preparative HPLC (Cis) using a gradient of acetonitrile in water (1 to 99% over 15 min) and HCl as a modifier. Evaporation gave 6-[[4-(2,6-dimethylphenyl)-5-methyl-6-[(2R)-4-methyl-2-(spiro[2.3]hexan-5-ylamino)pentoxy]pyrimidin-2-yl]sulfamoyl]pyridine-2-carboxylic acid (hydrochloride salt) (27 mg, 9%) as an off-white residue. ESI-MS m/z calc. 593.2672, found 594.46 (M+1)⁺; Retention time: 1.31 minutes; LC method A.

### Step 4: (11R)-6-(2,6-Dimethylphenyl)-7-methyl-11-(2-methylpropyl)-12-{spiro[2.3]hexan-5-yl}-9-oxa-2λ⁶-thia-3,5,12,18,19-pentaazatricyclo[12.3.1.14,8]nonadeca-1(17),4(19),5,7,14(18),15-hexaene-2,2,13-trione (Compound 118)

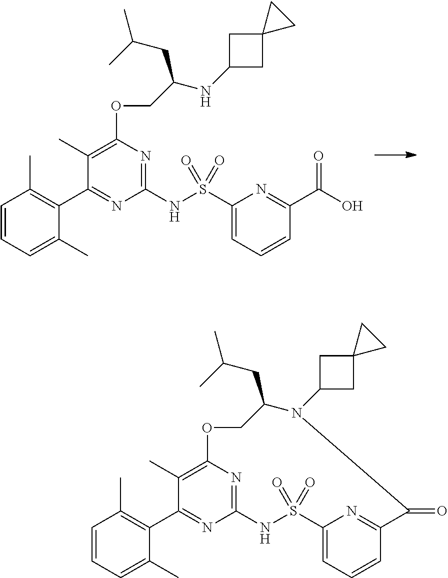 [SMILES: Cc1cccc(C)c1-c1nc(NS(=O)(=O)c2cccc(C(=O)O)n2)nc(OC[C@@H](CC(C)C)NC2CC3(CC3)C2)c1C ; Cc1cccc(C)c1-c1nc2nc(c1C)OC[C@@H](CC(C)C)N(C1CC3(CC3)C1)C(=O)c1cccc(n1)S(=O)(=O)N2]

A 20 mL flask was charged under nitrogen with HATU (45 mg, 0.1183 mmol), anhydrous DMF (2 mL) and DIEA (43 μL, 0.2469 mmol). A solution of 6-[[4-(2,6-dimethylphenyl)-5-methyl-6-[(2R)-4-methyl-2-(spiro[2.3]hexan-5-ylamino)pentoxy]pyrimidin-2-yl]sulfamoyl]pyridine-2-carboxylic acid (hydrochloride salt) (27 mg, 0.04284 mmol) in anhydrous DMF (1.5 mL) was added dropwise through syringe over a period of 4 minutes. The mixture was stirred at room temperature for 17 hours. The mixture was concentrated and diluted with DMSO (2 mL). The solution was microfiltered through a syringe filter disc and purified by reverse phase preparative HPLC (Cis) using a gradient of acetonitrile in water (1 to 99% over 15 min) and HCl as a modifier. Evaporation gave a residue that was triturated in DCM/hexanes. Evaporation of the solvents gave a residue that was triturated in DCM/hexanes. Evaporation of the solvents gave (11R)-6-(2,6-dimethylphenyl)-7-methyl-11-(2-methylpropyl)-12-{spiro[2.3]hexan-5-yl}-9-oxa-2λ⁶-thia-3,5,12,18,19-pentaazatricyclo[12.3.1.14,8]nonadeca-1(17),4(19),5,7,14(18),15-hexaene-2,2,13-trione (11 mg, 42%) as an off-white solid. ESI-MS m/z calc. 575.25665, found 576.64 (M+1)⁺; Retention time: 2.02 minutes; LC method A. ¹H NMR (500 MHz, DMSO-d₆) δ 12.99 (broad s, 1H), 8.16 (s, 1H), 8.03 (s, 1H), 7.79 (s, 1H), 7.28 (t, J=7.8 Hz, 1H), 7.18 (d, J=7.6 Hz, 1H), 7.13 (d, J=7.6 Hz, 1H), 5.61 (s, 1H), 4.29 (dq, J=20.6, 10.6, 9.6 Hz, 2H), 3.57 (ddt, J=10.9, 8.2, 4.2 Hz, 1H), 3.40-3.34 (m, 2H overlapped with water), 2.09 (m, 5H), 1.83 (s, 3H), 1.69 (ddd, J=14.0, 10.4, 3.0 Hz, 1H), 1.59 (s, 3H), 1.33-1.27 (m, 1H), 1.20-1.11 (m, 1H), 0.72 (d, J=6.6 Hz, 3H), 0.58-0.42 (m, 4H), 0.24 (d, J=6.4 Hz, 3H).

### Example 42: Preparation of Compound 119

### Step 1: 5-Bromo-4-chloro-6-(2,6-dimethylphenyl)pyrimidin-2-amine

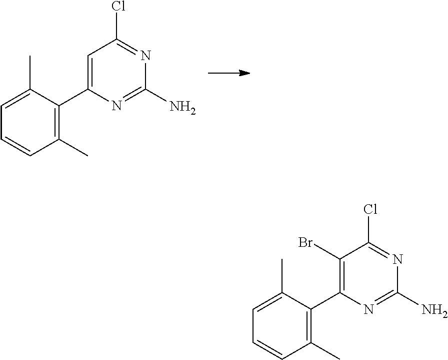 [SMILES: Cc1cccc(C)c1-c1cc(Cl)nc(N)n1 ; Cc1cccc(C)c1-c1nc(N)nc(Cl)c1Br]

To a solution of 4-chloro-6-(2,6-dimethylphenyl)pyrimidin-2-amine (1.0864 g, 4.6023 mmol) in glacial acetic acid (15 mL) at RT was added dropwise bromine (899.67 mg, 290 μL, 5.6297 mmol). The resulting solution was stirred at RT for 3.5 h. The reaction solution was filtered. The filtered solid was washed with hexanes (2×30 mL) and collected. The solid was dissolved in saturated aqueous sodium bicarbonate (15 mL) and extracted with EtOAc (3×20 mL). The combined organic layers were washed with 10% sodium thiosulfate aqueous solution (20 mL), saturated aqueous NaCl (2×20 mL), dried over anhydrous sodium sulfate, filtered and concentrated under the vacuum. The solid obtained was washed with hexanes (2×20 mL) and dried under high vacuum to afford a white solid. 5-bromo-4-chloro-6-(2,6-dimethylphenyl)pyrimidin-2-amine (1.0435 g, 71%). ESI-MS m/z calc. 310.98248, found 311.8 (M+1)⁺; Retention time: 5.65 minutes; LC method S.

### Step 2: Methyl 3-[[5-bromo-4-chloro-6-(2,6-dimethylphenyl)pyrimidin-2-yl]sulfamoyl]benzoate

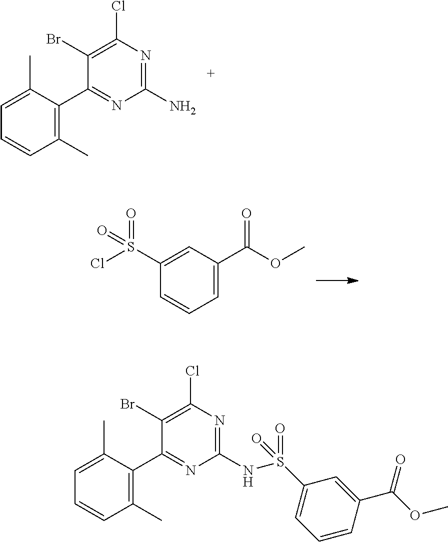 [SMILES: Cc1cccc(C)c1-c1nc(N)nc(Cl)c1Br ; COC(=O)c1cccc(S(=O)(=O)Cl)c1 ; COC(=O)c1cccc(S(=O)(=O)Nc2nc(Cl)c(Br)c(-c3c(C)cccc3C)n2)c1]

To a stirring solution of 5-bromo-4-chloro-6-(2,6-dimethylphenyl)pyrimidin-2-amine (20.06 g, 64.173 mmol) and methyl 3-chlorosulfonylbenzoate (22.834 g, 97.308 mmol) in anhydrous THF (450 mL) at 0° C. under nitrogen was dropwise added a solution of lithium tert-amoxide (37.376 g, 128 mL of 40% w/w, 158.91 mmol) in heptanes. After the addition was complete, the reaction mixture was stirred at this temperature for 2 hours. The reaction was quenched with aqueous HCl solution (600 mL, 1N)). The solution was extracted with ethyl acetate (3×400 mL). The combined organic layers were washed with brine (100 mL), dried over anhydrous sodium sulfate and concentrated under reduced pressure. The crude product obtained was purified by flash chromatography (half each for 2 times) (loaded in DCM) (330g silica gel, eluting 0 to 30% ethyl acetate in hexane). Pure fractions were concentrated under reduced pressure. Solid precipitated out and was filtered, which was combined with products in the filtrate to afford methyl 3-[[5-bromo-4-chloro-6-(2,6-dimethylphenyl)pyrimidin-2-yl]sulfamoyl]benzoate (26.46 g, 79%) as a white solid. ESI-MS m/z calc. 508.98117, found 510.1 (M+1)⁺; Retention time: 6.5 minutes; LC method S. ¹H NMR (500 MHz, DMSO-d₆) δ 12.63 (s, 1H), 8.38 (t, J=1.8 Hz, 1H), 8.20 (dt, J=7.8, 1.4 Hz, 1H), 8.11 (ddd, J=7.9, 2.0, 1.2 Hz, 1H), 7.70 (t, J=7.9 Hz, 1H), 7.27 (t, J=7.6 Hz, 1H), 7.11 (d, J=7.6 Hz, 2H), 3.82 (s, 3H), 1.74 (s, 6H).

### Step 3: 3-[[5-Bromo-4-chloro-6-(2,6-dimethylphenyl)pyrimidin-2-yl]sulfamoyl]benzoic acid

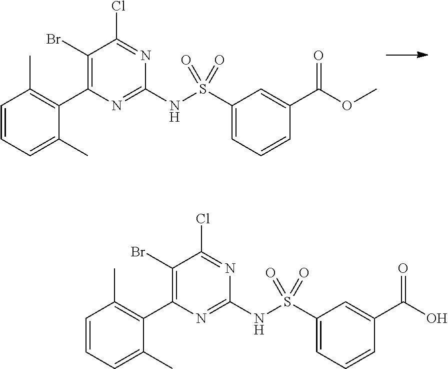 [SMILES: COC(=O)c1cccc(S(=O)(=O)Nc2nc(Cl)c(Br)c(-c3c(C)cccc3C)n2)c1 ; Cc1cccc(C)c1-c1nc(NS(=O)(=O)c2cccc(C(=O)O)c2)nc(Cl)c1Br]

To a solution of methyl 3-[[5-bromo-4-chloro-6-(2,6-dimethylphenyl)pyrimidin-2-yl]sulfamoyl]benzoate (23.08 g, 45.185 mmol) in THF (370 mL) was added NaOH (185 mL of 1 M, 185.00 mmol), and the resulting solution was stirred at ambient temperature for 100 minutes. Then water (150 mL) was added followed by the addition of aqueous solution HCl (6 N) dropwise until pH value of solution reached 1. The organic layer was separated, and aqueous layer was extracted with ethyl acetate (3×400 mL). The combined organic layers were washed with brine (100 mL×2), dried over anhydrous sodium sulfate, filtered and concentrated under reduced pressure. The solid was dissolved in dichloromethane (30 mL) and hexane (around 200 mL) was slowly added until white precipitate came out. The solution was heated to 40° C., filtered, washed with hexane and dried in high vacuum to afford 3-[[5-bromo-4-chloro-6-(2,6-dimethylphenyl)pyrimidin-2-yl]sulfamoyl]benzoic acid (19.4 g, 81%) as a white solid. ESI-MS m/z calc. 494.9655, found 496.1 (M+1)⁺; Retention time: 2.44 minutes; LC method W. ¹H NMR (500 MHz, DMSO-d₆) δ 13.44 (s, 1H), 12.60 (s, 1H), 8.41 (t, J=1.8 Hz, 1H), 8.19 (dt, J=7.8, 1.4 Hz, 1H), 8.13-8.04 (m, 1H), 7.67 (t, J=7.8 Hz, 1H), 7.26 (t, J=7.6 Hz, 1H), 7.12 (d, J=7.6 Hz, 2H), 1.77 (s, 6H).

### Step 4: 3-[[4-[(1R)-2-Amino-1-methyl-ethoxy]-5-bromo-6-(2,6-dimethylphenyl)pyrimidin-2-yl]sulfamoyl]benzoic acid

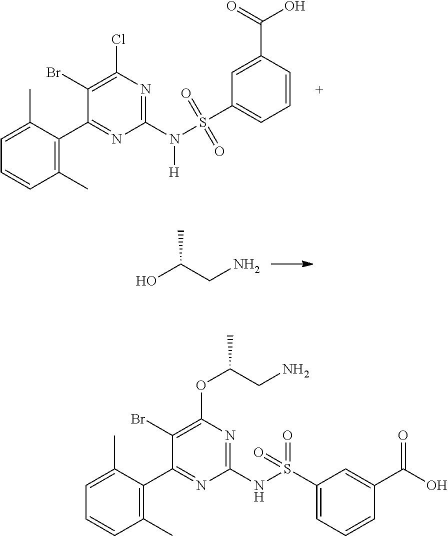

A solution of 3-[[5-bromo-4-chloro-6-(2,6-dimethylphenyl)pyrimidin-2-yl]sulfamoyl]benzoic acid (499.3 mg, 0.9549 mmol) in anhydrous THF (19 mL) was stirred at 0° C. for 10 min. To this solution was added (2R)-1-aminopropan-2-ol (88.817 mg, 95 μL, 1.1588 mmol), followed by Sodium tert-butoxide (377.7 mg, 3.8515 mmol). The reaction was stirred at that temperature for 1 h and 40 min. The reaction mixture was diluted with EtOAc (10 mL) and washed with aqueous 1 M HCl (10 mL). The aqueous 1 M HCl layer was extracted with EtOAc (2×20 mL). The combined organic layers were washed with saturated aqueous NaCl (10 mL), dried over anhydrous sodium sulfate, filtered and concentrated under vacuum. 3-[[4-[(1R)-2-amino-1-methyl-ethoxy]-5-bromo-6-(2,6-dimethylphenyl)pyrimidin-2-yl]sulfamoyl]benzoic acid (hydrochloride salt) (679.8 mg, 116%). ESI-MS m/z calc. 534.05725, found 535.1 (M+1)⁺; Retention time: 4.13 minutes; (LC method S).

### Step 5: (10R)-7-Bromo-6-(2,6-dimethylphenyl)-10-methyl-2,2-dioxo-9-oxa-2⁶-thia-3,5,12,19-tetrazatricyclo[12.3.1.14,8]nonadeca-1(18),4(19),5,7,14,16-hexaen-13-one

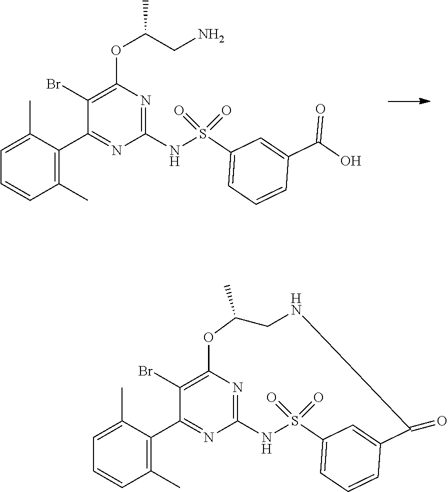 [SMILES: Cc1cccc(C)c1-c1nc(NS(=O)(=O)c2cccc(C(=O)O)c2)nc(O[C@H](C)CN)c1Br ; Cc1cccc(C)c1-c1nc2nc(c1Br)O[C@H](C)CNC(=O)c1cccc(c1)S(=O)(=O)N2]

A solution of 3-[[4-[(1R)-2-amino-1-methyl-ethoxy]-5-bromo-6-(2,6-dimethylphenyl)pyrimidin-2-yl]sulfamoyl]benzoic acid (hydrochloride salt) (3 g, 4.9836 mmol) and DIEA (7.4000 g, 10 mL, 57.256 mmol) in DMF (80 mL) was added dropwise to a solution of HATU (3.5 g, 9.2050 mmol) and HOBT (0.7 g, 5.1805 mmol) in DMF (80 mL). The reaction mixture was stirred at room temperature for 25 min. The reaction mixture was diluted with ethyl acetate and 1M HCl. The organic was separated, washed with brine, dried over sodium sulfate and concentrated. The residue was added DCM (40 mL)/Ether (100 mL) and filtered to give an off-white powder product (10R)-7-bromo-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (1.46 g, 54%). ESI-MS m/z calc. 516.0467, found 517.4 (M+1)⁺; Retention time: 4.53 minutes; (LC method S).

### Step 6: (10R)-6-(2,6-Dimethylphenyl)-10-methyl-2,2-dioxo-7-phenyl-9-oxa-2×⁶-thia-3,5,12,19-tetrazatricyclo[12.3.1.14,8]nonadeca-1(18),4(19),5,7,14,16-hexaen-13-one (Compound 119)

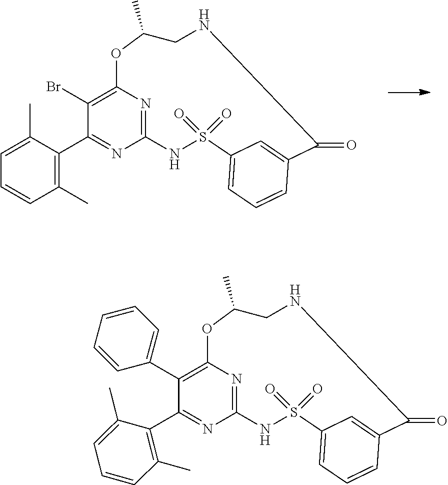 [SMILES: Cc1cccc(C)c1-c1nc2nc(c1Br)O[C@H](C)CNC(=O)c1cccc(c1)S(=O)(=O)N2 ; Cc1cccc(C)c1-c1nc2nc(c1-c1ccccc1)O[C@H](C)CNC(=O)c1cccc(c1)S(=O)(=O)N2]

To a solution of (10R)-7-bromo-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (70 mg, 0.1218 mmol) and 4,4,5,5-tetramethyl-2-phenyl-1,3,2-dioxaborolane (70 mg, 0.3331 mmol) in 1,4-Dioxane (2 mL) and 2M potassium carbonate (1 mL) was added Pd(dppf)Cl₂ (20 mg, 0.0245 mmol) at RT under nitrogen. The reaction mixture was stirred at 160° C. for 50 minutes under Microwave conditions. The mixture was added EtOAc (10 mL) and brine (20 mL). The organic layer was concentrated and dissolved in DMSO (2 mL). The crude product sample (in DMSO) was purified by HPLC using 20 to 80% acetonitrile in water buffered with 0.1% TFA to afford (10R)-6-(2,6-dimethylphenyl)-10-methyl-2,2-dioxo-7-phenyl-9-oxa-2λ⁶-thia-3,5,12,19-tetrazatricyclo[12.3.1.14,8]nonadeca-1(18),4(19),5,7,14,16-hexaen-13-one (28.9 mg, 45%) as off-white powder. ¹H NMR (500 MHz, DMSO-d₆) δ 8.52 (s, 1H), 8.26-8.16 (m, 1H), 7.96 (d, J=7.3 Hz, 1H), 7.76-7.65 (m, 2H), 7.17-7.06 (m, 4H), 7.02-6.88 (m, 4H), 5.51-5.39 (m, 1H), 3.46-3.30 (m, 1H), 2.79-2.65 (m, 1H), 2.03 (s, 3H), 1.92 (s, 3H), 1.35 (d, J=6.2 Hz, 3H). ESI-MS m/z calc. 514.1675, found 515.6 (M+1)⁺; Retention time: 2.01 minutes; LC method W.

### Example 43: Preparation of Compound 120 and Compound 121

### Step 1: (10R)-7-[(E)-3,3-Dimethylbut-1-enyl]-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 121)

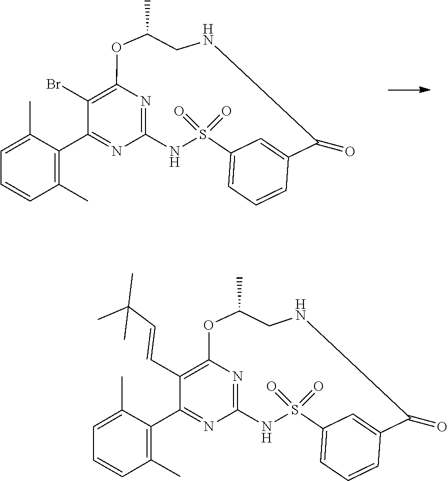 [SMILES: Cc1cccc(C)c1-c1nc2nc(c1Br)O[C@H](C)CNC(=O)c1cccc(c1)S(=O)(=O)N2 ; Cc1cccc(C)c1-c1nc2nc(c1/C=C/C(C)(C)C)O[C@H](C)CNC(=O)c1cccc(c1)S(=O)(=O)N2]

To a solution of (10R)-7-bromo-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (150 mg, 0.2754 mmol) and 2-[(E)-3,3-dimethylbut-1-enyl]-4,4,5,5-tetramethyl-1,3,2-dioxaborolane (160 mg, 0.7615 mmol) in 1,4-Dioxane (4 mL) and 2M potassium carbonate (2 mL) was added Pd(dppf)Cl₂ (40 mg, 0.0490 mmol) at RT under nitrogen. The reaction mixture was stirred at 160° C. for 45 minutes, then fresh Pd(dppf)Cl₂ (20 mg, 0.0245 mmol) was added and stirred another 20 min at 160° C. under microwave conditions. The mixture was added EtOAc (40 mL) and brine (20 mL). The organic layer was dried over sodium sulfate and concentrated yield about 300 mg crude product. The about half amount (100 mg) crude product sample was dissolved in DMSO (2 mL) and purified by HPLC using 20 to 80% acetonitrile in water buffered with 0.1% TFA to afford (10R)-7-[(E)-3,3-dimethylbut-1-enyl]-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (10.7 mg, 7%) as an off-white powder. ¹H NMR (500 MHz, DMSO-d₆) δ 13.03 (bs, 1H), 8.50 (s, 1H), 8.29 (s, 1H), 7.93 (s, 1H), 7.69 (s, 2H), 7.27 (d, J=8.2 Hz, 1H), 7.14 (dd, J=20.0, 7.6 Hz, 2H), 5.76 (s, 1H), 5.47 (s, 2H), 3.37-3.31 (m, 1H), 2.87-2.76 (m, 1H), 2.02 (s, 3H), 1.90 (s, 3H), 1.54 (d, J=6.2 Hz, 3H), 0.76 (s, 9H). ESI-MS m/z calc. 520.2144, found 521.5 (M+1)⁺; Retention time: 2.42 minutes; LC method W.

### Step 2: (10R)-7-(3,3-Dimethylbutyl)-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 120)

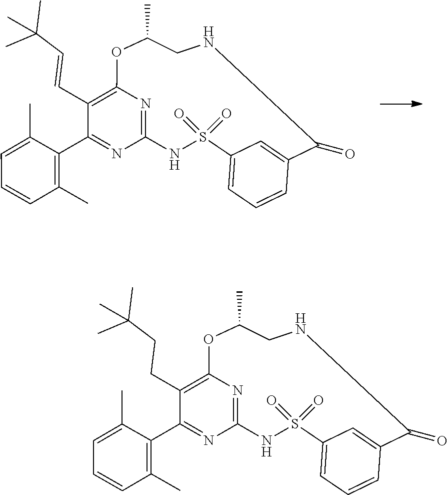 [SMILES: Cc1cccc(C)c1-c1nc2nc(c1/C=C/C(C)(C)C)O[C@H](C)CNC(=O)c1cccc(c1)S(=O)(=O)N2 ; Cc1cccc(C)c1-c1nc2nc(c1CCC(C)(C)C)O[C@H](C)CNC(=O)c1cccc(c1)S(=O)(=O)N2]

To a solution of (10R)-7-[(E)-3,3-dimethylbut-1-enyl]-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (100 mg, 0.1345 mmol) in MeOH (10 mL)/EtOAc (5 mL) and HOAc (1 mL) was added 10% palladium on carbon (50 mg). The mixture was hydrogenated in a Parr shaker at 60 psi for 3 days. The reaction mixture was filtered through Celite pad. The filtrate was concentrated and dissolved in DMSO (2 mL). The crude product sample (in DMSO) was purified by HPLC using 20 to 80% acetonitrile in water buffered with 0.1% TFA to afford (10R)-7-(3,3-dimethylbutyl)-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (16.2 mg, 23%) as a white powder. ¹H NMR (500 MHz, DMSO-d₆) δ 8.48 (s, 1H), 8.30-8.23 (m, 1H), 7.91 (d, J=7.4 Hz, 1H), 7.71-7.62 (m, 2H), 7.28 (t, J=7.6 Hz, 1H), 7.18 (d, J=7.7 Hz, 1H), 7.14 (d, J=7.6 Hz, 1H), 5.47-5.39 (m, 1H), 3.38-3.29 (m, 1H), 2.84-2.74 (m, 1H), 2.06 (s, 3H), 1.92 (s, 3H), 1.51 (d, J=6.2 Hz, 3H), 1.17-0.93 (m, 3H), 0.63 (s, 9H). ESI-MS m/z calc. 522.2301, found 523.3 (M+1)⁺; Retention time: 2.56 minutes; LC method W.

### Example 44: Preparation of Compound 122

### Step 1: (10R)-6-(2,6-Dimethylphenyl)-7-(3-methoxyphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 122)

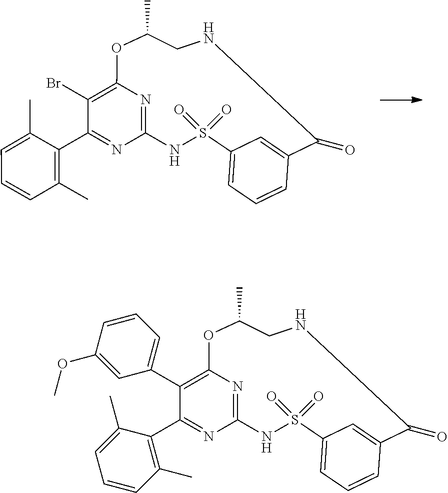 [SMILES: Cc1cccc(C)c1-c1nc2nc(c1Br)O[C@H](C)CNC(=O)c1cccc(c1)S(=O)(=O)N2 ; COc1cccc(-c2c3nc(nc2-c2c(C)cccc2C)NS(=O)(=O)c2cccc(c2)C(=O)NC[C@@H](C)O3)c1]

To a solution of (10R)-7-bromo-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (70 mg, 0.1218 mmol) and (3-methoxyphenyl)boronic acid (50 mg, 0.3290 mmol) in 1,4-Dioxane (2 mL), and 2M potassium carbonate (1 mL) was added Pd(dppf)Cl₂ (20 mg, 0.0245 mmol) at RT under nitrogen. The reaction mixture was stirred at 145° C. for 20 minutes, then fresh Pd(dppf)Cl₂ (20 mg, 0.0245 mmol) was added and another 1 hour at 120° C. under Microwave conditions. The mixture was added EtOAc (10 mL) and brine (20 mL). The organic layer was concentrated and dissolved in DMSO (2 mL). The crude product sample (in DMSO) was purified by HPLC using 20 to 80% acetonitrile in water buffered with 0.1% TFA to afford (10R)-6-(2,6-dimethylphenyl)-7-(3-methoxyphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (33.1 mg, 47%) as an off-white powder. ¹H NMR (500 MHz, DMSO-d₆) δ 8.52 (s, 1H), 8.21 (s, 1H), 7.96 (s, 1H), 7.76-7.67 (m, 2H), 7.12 (t, J=7.7 Hz, 1H), 7.05 (t, J=8.0 Hz, 1H), 6.96 (t, J=8.5 Hz, 2H), 6.72-6.67 (m, 1H), 6.60 (d, J=7.7 Hz, 1H), 6.53 (t, J=2.1 Hz, 1H), 5.46 (s, 1H), 3.53 (s, 3H), 3.39-3.36 (m, 1H), 2.09 (d, J=65.7 Hz, 3H), 1.94 (s, 3H), 1.37 (d, J=6.1 Hz, 3H). ESI-MS m/z calc. 544.17804, found 545.2 (M+1)⁺; Retention time: 2.1 minutes; LC method W.

### Example 45: Preparation of Compound 123

### Step 1: (11R)-6-(2,6-Dimethylphenyl)-7-(2-methoxyphenyl)-11-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 123)

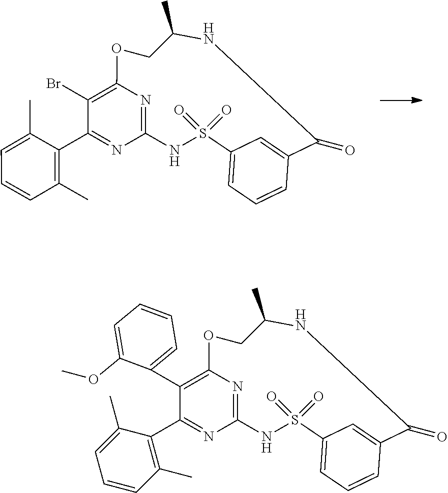 [SMILES: Cc1cccc(C)c1-c1nc2nc(c1Br)OC[C@@H](C)NC(=O)c1cccc(c1)S(=O)(=O)N2 ; COc1ccccc1-c1c2nc(nc1-c1c(C)cccc1C)NS(=O)(=O)c1cccc(c1)C(=O)N[C@H](C)CO2]

To a solution of (11R)-7-bromo-6-(2,6-dimethylphenyl)-11-methyl-2,2-dioxo-9-oxa-2λ⁶-thia-3,5,12,19-tetrazatricyclo[12.3.1.14,8]nonadeca-1(18),4(19),5,7,14,16-hexaen-13-one (94 mg, 0.1780 mmol) and (2-methoxyphenyl)boronic acid (80 mg, 0.5265 mmol) in 1,4-Dioxane (1.5 mL), ACN (1.5 mL) and 2M potassium carbonate (1.5 mL) was added Pd(dppf)Cl₂ (30 mg, 0.0367 mmol) at RT under nitrogen. The reaction mixture was stirred at 145° C. for 20 minutes, then fresh Pd(dppf)Cl₂ (30 mg, 0.0367 mmol) was added and stirred 20 min at 140° C. and another 1 hour at 120° C. under Microwave conditions. The mixture was added EtOAc (10 mL) and brine (20 mL). The organic layer was concentrated and dissolved in DMSO (2 mL). The crude product sample (in DMSO) was purified by HPLC using 20 to 80% acetonitrile in water buffered with 0.1% TFA to afford (11R)-6-(2,6-dimethylphenyl)-7-(2-methoxyphenyl)-11-methyl-2,2-dioxo-9-oxa-2λ⁶-thia-3,5,12,19-tetrazatricyclo[12.3.1.14,8]nonadeca-1(18),4(19),5,7,14,16-hexaen-13-one (48.6 mg, 49%) as an off-white powder. ¹H NMR (500 MHz, DMSO-d₆) δ 13.17 (bs, 1H), 8.54 (s, 1H), 8.02-7.82 (m, 2H), 7.69 (s, 2H), 7.21-7.13 (m, 1H), 7.11-6.96 (m, 2H), 6.91-6.80 (m, 3H), 6.77-6.66 (m, 1H), 5.16 (s, 1H), 3.71-3.64 (m, 1H), 3.58 (d, J=22.0 Hz, 3H), 3.37 (s, 1H), 2.20 (s, 2H), 2.09-1.95 (m, 2H), 1.82 (s, 2H), 1.07 (d, J=6.4 Hz, 2H), 0.94 (d, J=6.4 Hz, 1H). ESI-MS m/z calc. 544.17804, found 545.2 (M+1)⁺; Retention time: 2.03 minutes; LC method W.

### Example 46: Preparation of Compound 124

### Step 1: (11R)-6-(2,6-Dimethylphenyl)-7-(1-isobutylpyrazol-4-yl)-11-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 124)

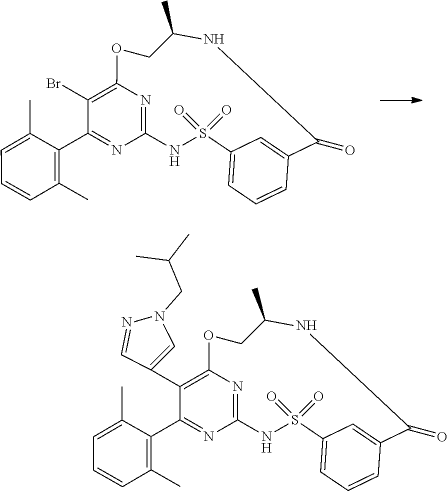 [SMILES: Cc1cccc(C)c1-c1nc2nc(c1Br)OC[C@@H](C)NC(=O)c1cccc(c1)S(=O)(=O)N2 ; Cc1cccc(C)c1-c1nc2nc(c1-c1cnn(CC(C)C)c1)OC[C@@H](C)NC(=O)c1cccc(c1)S(=O)(=O)N2]

To a solution of (11R)-7-bromo-6-(2,6-dimethylphenyl)-11-methyl-2,2-dioxo-9-oxa-2λ⁶-thia-3,5,12,19-tetrazatricyclo[12.3.1.14,8]nonadeca-1(18),4(19),5,7,14,16-hexaen-13-one (75 mg, 0.1421 mmol) and 1-isobutyl-4-(4,4,5,5-tetramethyl-1,3,2-dioxaborolan-2-yl)pyrazole (100 mg, 0.3998 mmol) in 1,4-Dioxane (2 mL) and 2M potassium carbonate (1.5 mL) was added Pd(dppf)Cl₂ (20 mg, 0.0245 mmol) at RT under nitrogen. The reaction mixture was stirred at 160° C. for 20 minutes under microwave conditions. The mixture was added EtOAc (20 mL) and brine (10 mL). The organic layer was concentrated and dissolved in DMSO (2 mL). The crude product sample (in DMSO) was purified by HPLC using 10 to 70% acetonitrile in 5 mM HCl water to afford (11R)-6-(2,6-dimethylphenyl)-7-(1-isobutylpyrazol-4-yl)-11-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (9.7 mg, 11%) as a white powder. ¹H NMR (500 MHz, DMSO-d₆) δ 8.57 (s, 1H), 7.99 (d, J=9.8 Hz, 1H), 7.94 (d, J=7.1 Hz, 1H), 7.69 (d, J=8.2 Hz, 2H), 7.23 (d, J=3.4 Hz, 2H), 7.09 (d, J=7.6 Hz, 1H), 7.05 (d, J=7.6 Hz, 1H), 6.87 (s, 1H), 5.29-5.22 (m, 1H), 3.91 (t, J=11.0 Hz, 1H), 3.74 (d, J=7.2 Hz, 3H), 3.49-3.45 (m, 1H), 1.99 (s, 3H), 1.90-1.79 (m, 4H), 1.08 (d, J=6.4 Hz, 3H), 0.62 (d, J=6.7 Hz, 6H). ESI-MS m/z calc. 560.2206, found 561.4 (M+1)⁺; Retention time: 2.1 minutes; LC method W.

### Example 47: Preparation of Compound 125

### Step 1: (11R)-7-(3,3-Dimethylbutyl)-6-(2,6-dimethylphenyl)-11-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 125)

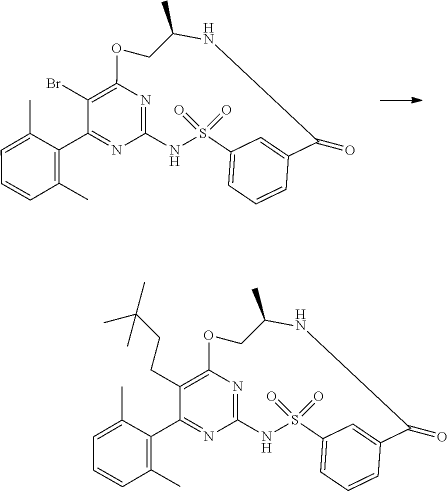 [SMILES: Cc1cccc(C)c1-c1nc2nc(c1Br)OC[C@@H](C)NC(=O)c1cccc(c1)S(=O)(=O)N2 ; Cc1cccc(C)c1-c1nc2nc(c1CCC(C)(C)C)OC[C@@H](C)NC(=O)c1cccc(c1)S(=O)(=O)N2]

To a solution of (11R)-7-bromo-6-(2,6-dimethylphenyl)-11-methyl-2,2-dioxo-9-oxa-2λ⁶-thia-3,5,12,19-tetrazatricyclo[12.3.1.14,8]nonadeca-1(18),4(19),5,7,14,16-hexaen-13-one (100 mg, 0.1643 mmol) and 2-[(E)-3,3-dimethylbut-1-enyl]-4,4,5,5-tetramethyl-1,3,2-dioxaborolane (100 mg, 0.4759 mmol) in 1,4-Dioxane (3 mL) and 2M potassium carbonate (1.5 mL) was added Pd(dppf)Cl₂ (30 mg, 0.0367 mmol) at RT under nitrogen. The reaction mixture was stirred at 160° C. for 45 minutes under Microwave conditions. The mixture was added EtOAc (50 mL) and brine (30 mL). The organic layer was dried over sodium sulfate and concentrated. To a solution of the residue (˜100 mg) in MeOH (10 mL)/EtOAc (5 mL) and TFA (0.5 mL) was added 10% palladium on carbon (50 mg). The mixture was hydrogenated in a Parr shaker at 60 psi for 2 days. Then the fresh Palladium on carbon (50 mg, 10% w/w, 0.0470 mmol) and was added and continually hydrogenated at 60 psi for another 2 days. the reaction mixture was filtered through Celite pad. The filtrate was concentrated and dissolved in DMSO (2 mL). The crude product sample (in DMSO) was purified by HPLC using 20 to 80% acetonitrile in water buffered with 0.1% TFA to afford (11R)-7-(3,3-dimethylbutyl)-6-(2,6-dimethylphenyl)-11-methyl-2,2-dioxo-9-oxa-2λ⁶-thia-3,5,12,19-tetrazatricyclo[12.3.1.14,8]nonadeca-1(18),4(19),5,7,14,16-hexaen-13-one (13.9 mg, 16%) as a white powder. ¹H NMR (500 MHz, DMSO-d₆) δ 8.52 (s, 1H), 8.05-7.82 (m, 2H), 7.66 (s, 2H), 7.38-7.03 (m, 3H), 5.31-5.12 (m, 1H), 3.87 (t, J=10.9 Hz, 1H), 2.05 (s, 4H), 1.99-1.84 (m, 4H), 1.14 (d, J=6.4 Hz, 3H), 1.11-0.97 (m, 2H), 0.61 (s, 9H). ESI-MS m/z calc. 522.2301, found 523.6 (M+1)⁺; Retention time: 2.45 minutes; LC method W.

### Example 48: Preparation of Compound 126

### Step 1: 3-[[4-[(2R)-2-Amino-4-methyl-pentoxy]-5-bromo-6-(2,6-dimethylphenyl)pyrimidin-2-yl]sulfamoyl]benzoic acid

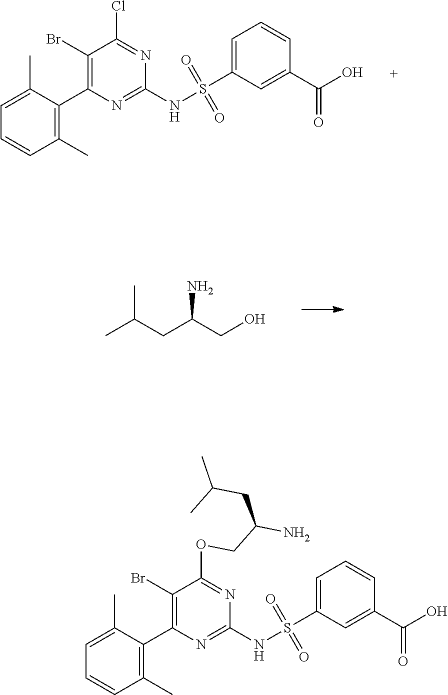

3-[[5-Bromo-4-chloro-6-(2,6-dimethylphenyl)pyrimidin-2-yl]sulfamoyl]benzoic acid (150 mg, 0.3020 mmol), (2R)-2-amino-4-methyl-pentan-1-ol (37.6 mg, 0.3208 mmol), and sodium tert-butoxide (117 mg, 1.217 mmol) were combined in THF (0.5 mL) and stirred at room temperature for 30 min. The reaction mixture was diluted with ethyl acetate and washed with 1M HCl. The organics were separated, washed with brine, dried over sodium sulfate and evaporated to give 3-[[4-[(2R)-2-amino-4-methyl-pentoxy]-5-bromo-6-(2,6-dimethylphenyl)pyrimidin-2-yl]sulfamoyl]benzoic acid (hydrochloride salt) (172 mg, 93%). ESI-MS m/z calc. 576.1042, found 578.8 (M+1)⁺; Retention time: 0.53 minutes; LC method D.

### Step 2: (11R)-7-Bromo-6-(2,6-dimethylphenyl)-11-(2-methylpropyl)-9-oxa-2λ⁶-thia-3,5,12,19-tetraazatricyclo[12.3.1.14,8]nonadeca-1(18),4,6,8(19),14,16-hexaene-2,2,13-trione

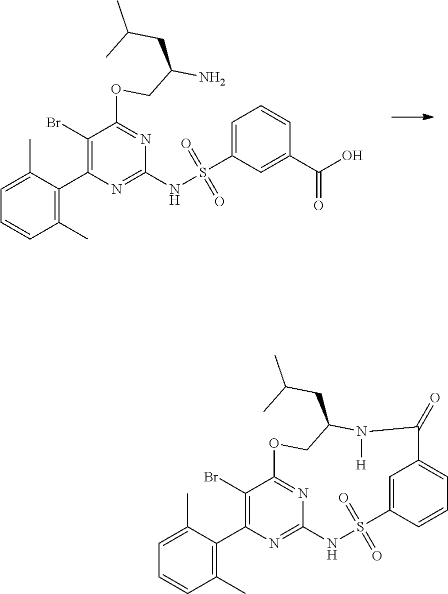 [SMILES: Cc1cccc(C)c1-c1nc(NS(=O)(=O)c2cccc(C(=O)O)c2)nc(OC[C@H](N)CC(C)C)c1Br ; Cc1cccc(C)c1-c1nc2nc(c1Br)OC[C@@H](CC(C)C)NC(=O)c1cccc(c1)S(=O)(=O)N2]

3-[[4-[(2R)-2-amino-4-methyl-pentoxy]-5-bromo-6-(2,6-dimethylphenyl)pyrimidin-2-yl]sulfamoyl]benzoic acid (hydrochloride salt) (172 mg, 0.2802 mmol), HATU (119.4 mg, 0.3140 mmol), and triethylamine (200 μL, 1.435 mmol) were combined in DMF (5 mL) and stirred at room temperature for 3 h. The reaction was partitioned between ethyl acetate and a 1M HCl solution. The organics were separated, washed with brine, dried over sodium sulfate and evaporated. The product was left crude as an off-white solid. (11R)-7-bromo-6-(2,6-dimethylphenyl)-11-(2-methylpropyl)-9-oxa-2λ⁶-thia-3,5,12,19-tetraazatricyclo[12.3.1.14,8]nonadeca-1(18),4,6,8(19),14,16-hexaene-2,2,13-trione (172 mg, 80%). ESI-MS m/z calc. 558.0936, found 559.3 (M+1)⁺; Retention time: 0.67 minutes; LC method D.

### Step 3: (11R)-7-[(1E)-3,3-dimethylbut-1-en-1-yl]-6-(2,6-dimethylphenyl)-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (Compound 126)

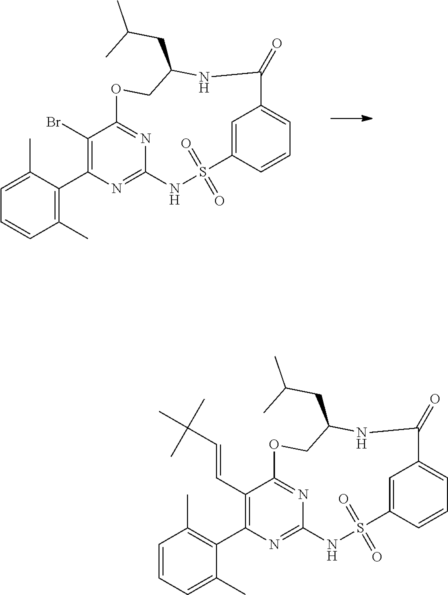 [SMILES: Cc1cccc(C)c1-c1nc2nc(c1Br)OC[C@@H](CC(C)C)NC(=O)c1cccc(c1)S(=O)(=O)N2 ; Cc1cccc(C)c1-c1nc2nc(c1/C=C/C(C)(C)C)OC[C@@H](CC(C)C)NC(=O)c1cccc(c1)S(=O)(=O)N2]

(11R)-7-Bromo-6-(2,6-dimethylphenyl)-11-(2-methylpropyl)-9-oxa-2λ⁶-thia-3,5,12,19-tetraazatricyclo[12.3.1.14,8]nonadeca-1(18),4,6,8(19),14,16-hexaene-2,2,13-trione (40 mg, 0.05219 mmol), 2-[(E)-3,3-dimethylbut-1-enyl]-4,4,5,5-tetramethyl-1,3,2-dioxaborolane (approximately 32.90 mg, 0.1566 mmol), aqueous potassium carbonate (approximately 104.4 μL of 2 M, 0.2088 mmol), and dichloro[1,1′-bis(diphenylphosphino)ferrocene]palladium (II) dichloromethane adduct (approximately 42.62 mg, 0.05219 mmol) were combined in dioxane (1 mL) and heated in the microwave for 20 min at 120° C. The reaction was filtered and purified by reverse-phase HPLC utilizing a gradient of 10-99% acetonitrile in 5 mM aqueous HCl to yield (11R)-7-[(1E)-3,3-dimethylbut-1-en-1-yl]-6-(2,6-dimethylphenyl)-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (3.3 mg, 11%). ESI-MS m/z calc. 562.26135, found 563.6 (M+1)⁺; Retention time: 1.91 minutes; LC method A.

### Example 49: Characterization of Compounds 127-155

The compounds in the following tables were prepared in a manner analogous to that described above using commercially available reagents and intermediates described herein.

TABLE 11

| Compound | | LCMS | Calc. | | LCMS |
|---|---|---|---|---|---|
| Number | Structure | Rt (min) | Mass | M + 1 | Method |
| 127 | 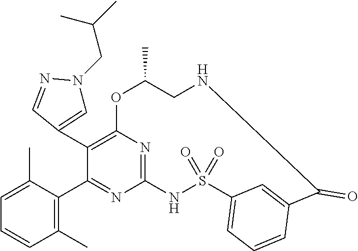 [SMILES: Cc1cccc(C)c1-c1nc2nc(c1-c1cnn(CC(C)C)c1)O[C@H](C)CNC(=O)c1cccc(c1)S(=O)(=O)N2] | 1.96 | 560.221 | 561.1 | W |
| 121 | 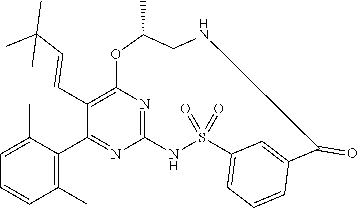 [SMILES: Cc1cccc(C)c1-c1nc2nc(c1/C=C/C(C)(C)C)O[C@H](C)CNC(=O)c1cccc(c1)S(=O)(=O)N2] | 2.42 | 520.214 | 521.5 | W |
| 128 | 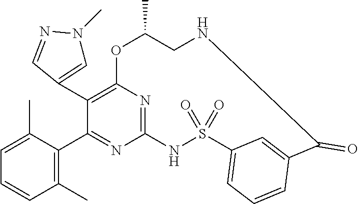 [SMILES: Cc1cccc(C)c1-c1nc2nc(c1-c1cnn(C)c1)O[C@H](C)CNC(=O)c1cccc(c1)S(=O)(=O)N2] | 1.53 | 518.174 | 519.1 | W |
| 129 | 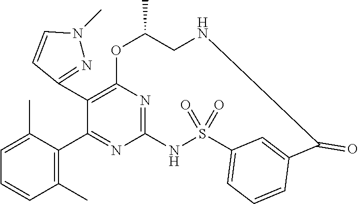 [SMILES: Cc1cccc(C)c1-c1nc2nc(c1-c1ccn(C)n1)O[C@H](C)CNC(=O)c1cccc(c1)S(=O)(=O)N2] | 1.59 | 518.174 | 519.4 | W |
| 130 | 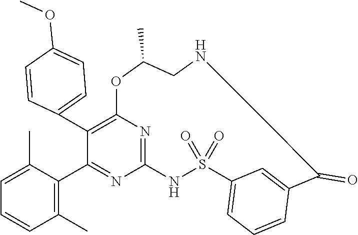 [SMILES: COc1ccc(-c2c3nc(nc2-c2c(C)cccc2C)NS(=O)(=O)c2cccc(c2)C(=O)NC[C@@H](C)O3)cc1] | 1.98 | 544.178 | 545.2 | W |
| 131 | 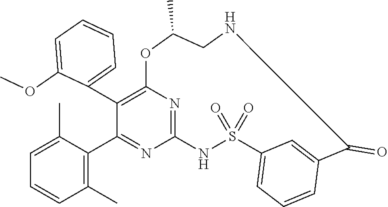 [SMILES: COc1ccccc1-c1c2nc(nc1-c1c(C)cccc1C)NS(=O)(=O)c1cccc(c1)C(=O)NC[C@@H](C)O2] | 2 | 544.178 | 545.2 | W |
| 132 | 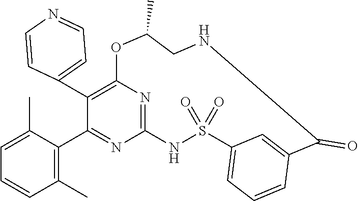 [SMILES: Cc1cccc(C)c1-c1nc2nc(c1-c1ccncc1)O[C@H](C)CNC(=O)c1cccc(c1)S(=O)(=O)N2] | 1.14 | 515.163 | 516.4 | W |
| 133 | 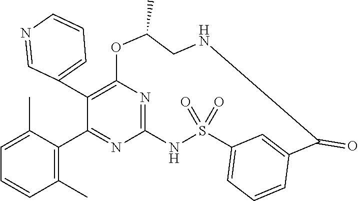 [SMILES: Cc1cccc(C)c1-c1nc2nc(c1-c1cccnc1)O[C@H](C)CNC(=O)c1cccc(c1)S(=O)(=O)N2] | 1.19 | 515.163 | 516.4 | W |
| 134 | 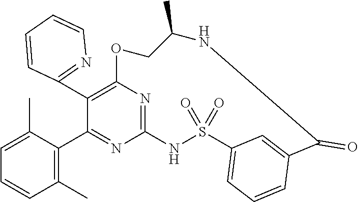 [SMILES: Cc1cccc(C)c1-c1nc2nc(c1-c1ccccn1)OC[C@@H](C)NC(=O)c1cccc(c1)S(=O)(=O)N2] | 1.47 | 515.163 | 516.4 | W |
| 135 | 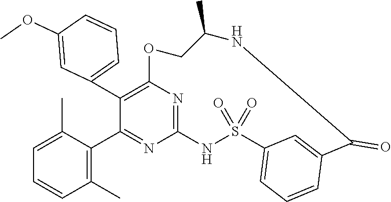 [SMILES: COc1cccc(-c2c3nc(nc2-c2c(C)cccc2C)NS(=O)(=O)c2cccc(c2)C(=O)N[C@H](C)CO3)c1] | 2.14 | 544.178 | | W |
| 136 | 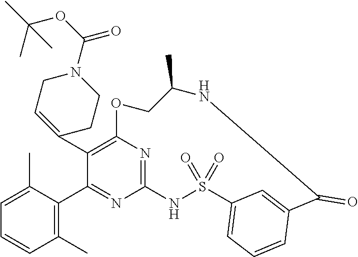 [SMILES: Cc1cccc(C)c1-c1nc2nc(c1C1=CCN(C(=O)OC(C)(C)C)CC1)OC[C@@H](C)NC(=O)c1cccc(c1)S(=O)(=O)N2] | 2.33 | 619.246 | 620.6 | W |
| 137 | 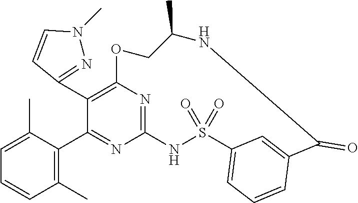 [SMILES: Cc1cccc(C)c1-c1nc2nc(c1-c1ccn(C)n1)OC[C@@H](C)NC(=O)c1cccc(c1)S(=O)(=O)N2] | 1.71 | 518.174 | 519.4 | W |
| 138 | 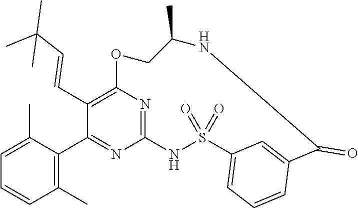 [SMILES: Cc1cccc(C)c1-c1nc2nc(c1/C=C/C(C)(C)C)OC[C@@H](C)NC(=O)c1cccc(c1)S(=O)(=O)N2] | 2.51 5 | 520.214 | 521.5 | W |
| 139 | 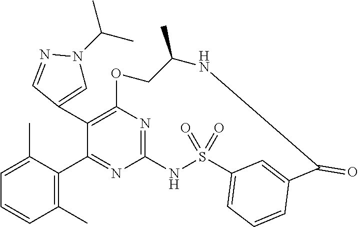 [SMILES: Cc1cccc(C)c1-c1nc2nc(c1-c1cnn(C(C)C)c1)OC[C@@H](C)NC(=O)c1cccc(c1)S(=O)(=O)N2] | 1.95 | 546.205 | 547.6 | W |
| 140 | 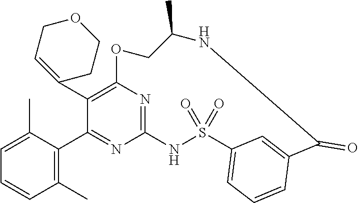 [SMILES: Cc1cccc(C)c1-c1nc2nc(c1C1=CCOCC1)OC[C@@H](C)NC(=O)c1cccc(c1)S(=O)(=O)N2] | 1.82 | 520.178 | 521.5 | W |
| 141 | 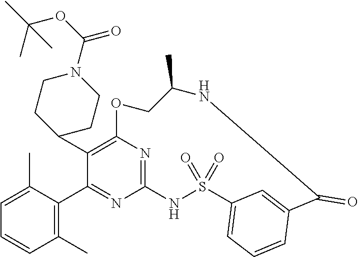 [SMILES: Cc1cccc(C)c1-c1nc2nc(c1C1CCN(C(=O)OC(C)(C)C)CC1)OC[C@@H](C)NC(=O)c1cccc(c1)S(=O)(=O)N2] | 2.85 | 621.262 | 622.7 | W |
| 142 | 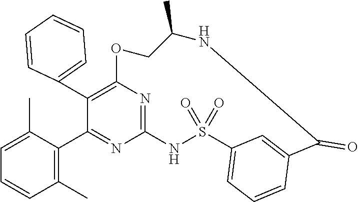 [SMILES: Cc1cccc(C)c1-c1nc2nc(c1-c1ccccc1)OC[C@@H](C)NC(=O)c1cccc(c1)S(=O)(=O)N2] | 2.04 | 514.167 | 515.5 | W |
| 143 | 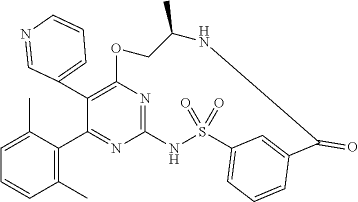 [SMILES: Cc1cccc(C)c1-c1nc2nc(c1-c1cccnc1)OC[C@@H](C)NC(=O)c1cccc(c1)S(=O)(=O)N2] | 1.31 | 515.163 | 516.4 | W |
| 144 | 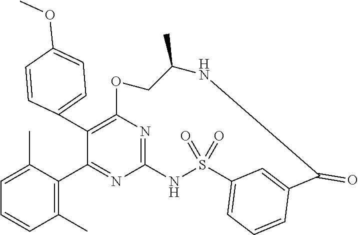 [SMILES: COc1ccc(-c2c3nc(nc2-c2c(C)cccc2C)NS(=O)(=O)c2cccc(c2)C(=O)N[C@H](C)CO3)cc1] | 2.03 | 544.178 | | W |
| 145 | 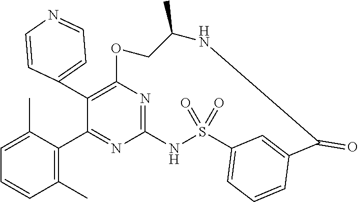 [SMILES: Cc1cccc(C)c1-c1nc2nc(c1-c1ccncc1)OC[C@@H](C)NC(=O)c1cccc(c1)S(=O)(=O)N2] | 1.22 | 515.163 | 516.4 | W |
| 146 | 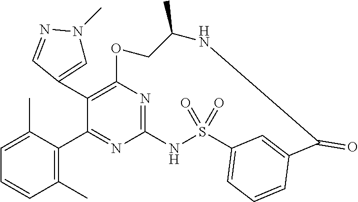 [SMILES: Cc1cccc(C)c1-c1nc2nc(c1-c1cnn(C)c1)OC[C@@H](C)NC(=O)c1cccc(c1)S(=O)(=O)N2] | 1.59 | 518.174 | 519.1 | W |
| 147 | 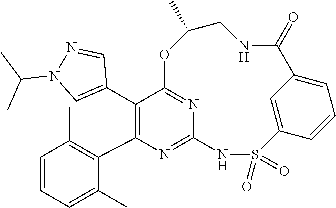 [SMILES: Cc1cccc(C)c1-c1nc2nc(c1-c1cnn(C(C)C)c1)O[C@H](C)CNC(=O)c1cccc(c1)S(=O)(=O)N2] | 1.28 | 546.205 | 547 | A |
| 148 | 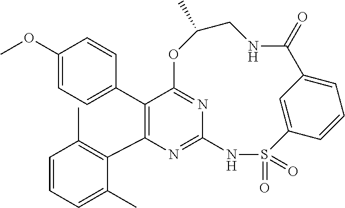 [SMILES: COc1ccc(-c2c3nc(nc2-c2c(C)cccc2C)NS(=O)(=O)c2cccc(c2)C(=O)NC[C@@H](C)O3)cc1] | 1.39 | 544.178 | 545 | A |
| 149 | 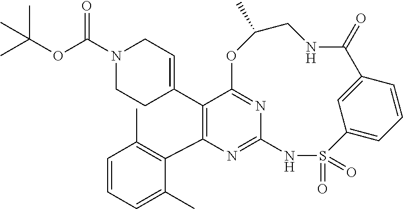 [SMILES: Cc1cccc(C)c1-c1nc2nc(c1C1=CCN(C(=O)OC(C)(C)C)CC1)O[C@H](C)CNC(=O)c1cccc(c1)S(=O)(=O)N2] | 1.6 | 619.246 | 620 | A |
| 150 | 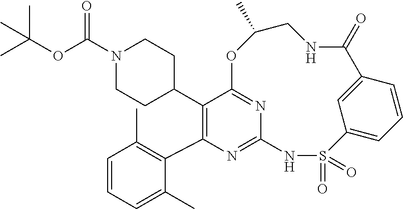 [SMILES: Cc1cccc(C)c1-c1nc2nc(c1C1CCN(C(=O)OC(C)(C)C)CC1)O[C@H](C)CNC(=O)c1cccc(c1)S(=O)(=O)N2] | 1.62 | 621.262 | 622 | A |
| 151 | 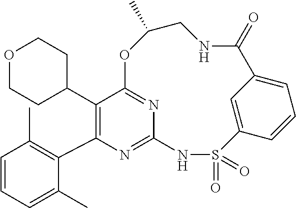 [SMILES: Cc1cccc(C)c1-c1nc2nc(c1C1CCOCC1)O[C@H](C)CNC(=O)c1cccc(c1)S(=O)(=O)N2] | 1.6 | 522.194 | 523 | I |
| 152 | 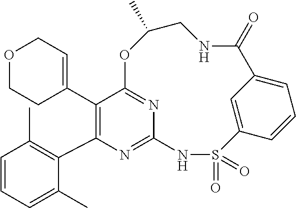 [SMILES: Cc1cccc(C)c1-c1nc2nc(c1C1=CCOCC1)O[C@H](C)CNC(=O)c1cccc(c1)S(=O)(=O)N2] | 1.2 | 520.178 | 521 | A |
| 153 | 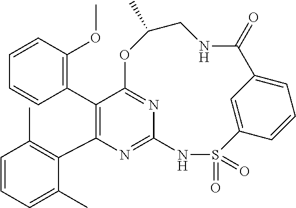 [SMILES: COc1ccccc1-c1c2nc(nc1-c1c(C)cccc1C)NS(=O)(=O)c1cccc(c1)C(=O)NC[C@@H](C)O2] | 1.94 | 544.178 | 545 | I |
| 154 | 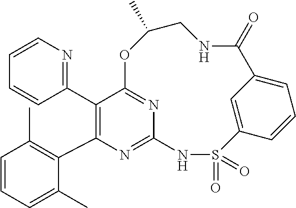 [SMILES: Cc1cccc(C)c1-c1nc2nc(c1-c1ccccn1)O[C@H](C)CNC(=O)c1cccc(c1)S(=O)(=O)N2] | 1.33 | 515.163 | 516 | I |
| 155 | 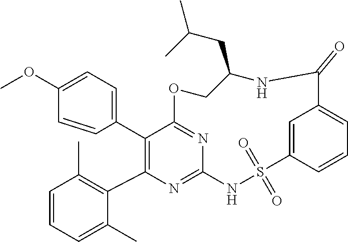 [SMILES: COc1ccc(-c2c3nc(nc2-c2c(C)cccc2C)NS(=O)(=O)c2cccc(c2)C(=O)N[C@H](CC(C)C)CO3)cc1] | 1.59 | 586.225 | 587.5 | A |

TABLE 12

| Compound | |
|---|---|
| Number | NMR |
| 127 | ¹H NMR (500 MHz, DMSO-d₆) δ 13.06 (bs, 1H), 8.58-8.45 (m, 1H), 8.35-8.24 |
| | (m, 1H), 8.01-7.87 (m, 1H), 7.81-7.59 (m, 2H), 7.28-7.20 (m, 1H), 7.19 (s, |
| | 1H), 7.14-7.01 (m, 2H), 6.82 (s, 1H), 5.55-5.41 (m, 1H), 3.74 (d, J = 7.2 Hz, |
| | 2H), 3.41-3.35 (m, 1H), 2.92-2.81 (m, 1H), 1.99 (s, 3H), 1.91-1.79 (m, 4H), |
| | 1.52 (d, J = 6.2 Hz, 3H), 0.64 (d, J = 6.6 Hz, 6H). |
| 121 | ¹H NMR (500 MHz, DMSO-d₆) δ 13.03 (bs, 1H), 8.50 (s, 1H), 8.29 (s, 1H), 7.93 |
| | (s, 1H), 7.69 (s, 2H), 7.27 (d, J = 8.2 Hz, 1H), 7.14 (dd, J = 20.0, 7.6 Hz, 2H), |
| | 5.76 (s, 1H), 5.47 (s, 2H), 3.37-3.31 (m, 1H), 2.87-2.76 (m, 1H), 2.02 (s, 3H), |
| | 1.90 (s, 3H), 1.54 (d, J = 6.2 Hz, 3H), 0.76 (s, 9H). |
| 128 | ¹H NMR (500 MHz, DMSO-d₆) δ 13.07 (bs, 1H), 8.52 (s, 1H), 8.32 (s, 1H), 7.94 |
| | (s, 1H), 7.78-7.64 (m, 2H), 7.36 (s, 1H), 7.27 (s, 1H), 7.11 (dd, J = 21.8, 7.6 Hz, |
| | 2H), 6.64 (s, 1H), 5.50 (s, 1H), 3.68 (s, 3H), 3.39-3.37 (m, 1H), 2.93-2.83 (m, |
| | 1H), 1.99 (s, 3H), 1.85 (s, 3H), 1.55 (d, J = 6.2 Hz, 3H). |
| 129 | ¹H NMR (500 MHz, DMSO-d₆) δ 13.06 (bs, 1H), 8.50 (s, 1H), 8.23 (s, 1H), 7.94 |
| | (s, 1H), 7.69 (s, 2H), 7.46 (d, J = 2.2 Hz, 1H), 7.12 (s, 1H), 6.96 (dd, J = 13.8, 7.7 |
| | Hz, 2H), 6.04 (d, J = 2.2 Hz, 1H), 5.51-5.38 (m, 1H), 3.54 (s, 3H), 3.30 (d, J = |
| | 10.0 Hz, 1H), 2.83-2.71 (m, 1H), 2.01 (s, 3H), 1.91 (s, 3H), 1.42 (d, J = 6.2 Hz, 3H). |
| 130 | ¹H NMR (500 MHz, DMSO-d₆) δ 8.51 (s, 1H), 8.22 (s, 1H), 7.95 (d, J = 6.8 Hz, |
| | 1H), 7.70 (s, 2H), 7.66-7.50 (m, 1H), 7.12 (t, J = 7.5 Hz, 1H), 7.00-6.92 (m, |
| | 2H), 6.91 (d, J = 8.8 Hz, 2H), 6.69 (d, J = 8.8 Hz, 2H), 5.45 (s, 1H), 3.41-3.33 |
| | (m, 1H), 2.79-2.66 (m, 1H), 2.02 (s, 3H), 1.91 (s, 3H), 1.36 (d, J = 6.2 Hz, 3H). |
| 131 | ¹H NMR (500 MHz, DMSO-d₆) δ 8.50 (d, J = 17.7 Hz, 1H), 8.27-8.13 (m, 1H), |
| | 7.96 (s, 1H), 7.70 (d, J = 19.8 Hz, 2H), 7.19-7.12 (m, 1H), 7.11-6.94 (m, 2H), |
| | 6.94-6.80 (m, 2H), 6.74-6.60 (m, 2H), 5.43 (s, 1H), 3.70 (s, 1H), 3.59 (s, 2H), |
| | 3.34-3.26 (m, 1H), 2.75-2.58 (m, 1H), 2.24-2.03 (m, 3H), 1.95-1.69 (m, 3H), |
| | 1.30 (dd, J = 19.2, 6.2 Hz, 3H). |
| 132 | ¹H NMR (500 MHz, DMSO-d₆) δ 8.54 (ddd, J = 7.3, 4.3, 1.8 Hz, 3H), 8.26 (dd, |
| | J = 9.8, 5.2 Hz, 1H), 8.02-7.96 (m, 1H), 7.76 (d, J = 7.6 Hz, 1H), 7.73 (t, J = |
| | 7.6 Hz, 1H), 7.38 (d, J = 5.8 Hz, 2H), 7.17 (t, J = 7.6 Hz, 1H), 6.99 (dd, J = 13.6, |
| | 7.6 Hz, 2H), 5.50-5.42 (m, 1H), 3.44-3.38 (m, 1H), 2.80-2.71 (m, 1H), 2.03 |
| | (s, 3H), 1.94 (s, 3H), 1.38 (d, J = 6.2 Hz, 3H). |
| 133 | ¹H NMR (500 MHz, DMSO-d₆) δ 8.53 (d, J = 1.9 Hz, 1H), 8.42 (dd, J = 5.0, 1.6 |
| | Hz, 1H), 8.28-8.20 (m, 2H), 8.01-7.95 (m, 1H), 7.74-7.68 (m, 2H), 7.65 (d, J = |
| | 8.0 Hz, 1H), 7.41-7.35 (m, 1H), 7.14 (t, J = 7.6 Hz, 1H), 6.98 (dd, J = 12.9, |
| | 7.6 Hz, 2H), 5.49-5.41 (m, 1H), 3.43-3.37 (m, 1H), 2.79-2.70 (m, 1H), 2.03 |
| | (s, 3H), 1.93 (s, 3H), 1.36 (d, J = 6.2 Hz, 3H). |
| 134 | ¹H NMR (500 MHz, DMSO-d₆) δ 8.56 (s, 1H), 8.41-8.34 (m, 1H), 8.00-7.87 |
| | (m, 2H), 7.71 (s, 2H), 7.62 (td, J = 7.7, 1.8 Hz, 1H), 7.19-7.13 (m, 2H), 7.06 (t, |
| | J = 7.6 Hz, 1H), 6.97 (d, J = 7.6 Hz, 1H), 6.85 (d, J = 7.6 Hz, 1H), 5.29-5.12 |
| | (m, 1H), 3.71 (t, J = 11.0 Hz, 1H), 2.15 (s, 3H), 1.89 (s, 3H), 1.05 (d, J = 6.4 Hz, 3H). |
| 135 | ¹H NMR (500 MHz, DMSO-d₆) δ 13.08 (bs, 1H), 8.56 (s, 1H), 7.94 (s, 1H), 7.88 |
| | (d, J = 9.8 Hz, 1H), 7.69 (s, 1H), 7.07 (t, J = 8.0 Hz, 2H), 7.01 (d, J = 7.6 Hz, |
| | 1H), 6.88 (d, J = 7.6 Hz, 1H), 6.71 (dd, J = 8.3, 2.6 Hz, 1H), 6.63-6.56 (m, 2H), |
| | 5.22-5.17 (m, 1H), 3.72 (t, J = 10.8 Hz, 1H), 3.56 (s, 3H), 3.54-3.48 (m, 1H), |
| | 2.18-2.10 (m, 3H), 1.96-1.81 (m, 3H), 1.06 (d, J = 6.4 Hz, 3H). |
| 136 | ¹H NMR (500 MHz, DMSO-d₆) δ 8.53 (s, 1H), 7.93 (dd, J = 16.6, 8.4 Hz, 2H), |
| | 7.68 (s, 2H), 7.21 (t, J = 7.6 Hz, 1H), 7.10 (d, J = 7.7 Hz, 1H), 7.05 (d, J = 7.6 |
| | Hz, 1H), 5.38 (s, 1H), 5.20 (d, J = 10.3 Hz, 1H), 3.88-3.81 (m, 1H), 3.66-3.64 |
| | (m, 1H), 3.57-3.42 (m, 3H), 3.22-3.05 (m, 2H), 2.08 (s, 3H), 1.97-1.88 (m, |
| | 4H), 1.33 (s, 9H), 1.10 (d, J = 6.4 Hz, 3H). |
| 137 | ¹H NMR (500 MHz, DMSO-d₆) δ 8.55 (s, 1H), 7.93 (d, J = 9.1 Hz, 2H), 7.69 (s, |
| | 1H), 7.45 (d, J = 2.2 Hz, 1H), 7.12 (d, J = 7.6 Hz, 1H), 7.01 (d, J = 7.6 Hz, 1H), |
| | 6.91 (d, J = 7.5 Hz, 1H), 5.86 (d, J = 2.2 Hz, 1H), 5.21 (d, J = 10.4 Hz, 1H), 3.77 |
| | (t, J = 10.9 Hz, 1H), 3.61 (s, 3H), 3.46-3.42 (m, 1H), 2.12 (d, J = 14.6 Hz, 3H), |
| | 1.84 (s, 3H), 1.10 (d, J = 6.4 Hz, 3H). |
| 138 | ¹H NMR (500 MHz, DMSO-d₆) δ 8.53 (s, 1H), 8.01 (d, J = 9.7 Hz, 1H), 7.92 (d, |
| | J = 7.1 Hz, 1H), 7.69 (s, 2H), 7.27 (t, J = 7.6 Hz, 1H), 7.16 (d, J = 7.6 Hz, 1H), |
| | 7.11 (d, J = 7.6 Hz, 1H), 5.74 (d, J = 16.5 Hz, 1H), 5.58 (d, J = 16.5 Hz, 1H), |
| | 5.27 (dd, J = 11.0, 3.7 Hz, 1H), 3.93 (t, J = 11.0 Hz, 1H), 3.46-3.38 (m, 1H), |
| | 2.03 (s, 3H), 1.90 (s, 3H), 1.11 (d, J = 6.4 Hz, 3H), 0.75 (s, 9H). |
| 139 | ¹H NMR (500 MHz, DMSO-d₆) δ 8.57 (s, 1H), 8.03 (d, J = 9.7 Hz, 1H), 7.94 (d, |
| | J = 7.2 Hz, 1H), 7.70 (s, 2H), 7.27 (t, J = 7.6 Hz, 1H), 7.20 (s, 1H), 7.14 (d, J = |
| | 7.6 Hz, 1H), 7.08 (d, J = 7.6 Hz, 1H), 6.83 (s, 1H), 5.30-5.22 (m, 1H), 4.36- |
| | 4.27 (m, 1H), 3.96 (t, J = 11.0 Hz, 1H), 3.52-3.50 (m, 1H), 2.00 (s, 3H), 1.84 (s, |
| | 3H), 1.22 (d, J = 2.0 Hz, 3H), 1.21 (d, J = 2.1 Hz, 3H), 1.11 (d, J = 6.4 Hz, 3H). |
| 140 | ¹H NMR (500 MHz, DMSO-d₆) δ 8.54 (s, 1H), 7.94 (dd, J = 18.2, 8.5 Hz, 2H), |
| | 7.67 (d, J = 8.3 Hz, 2H), 7.24 (q, J = 7.7 Hz, 1H), 7.09 (dd, J = 19.3, 7.7 Hz, 2H), |
| | 5.47-5.39 (m, 1H), 5.24-5.16 (m, 1H), 3.90-3.79 (m, 4H), 3.51-3.40 (m, |
| | 3H), 2.08 (s, 3H), 1.98-1.84 (m, 4H), 1.10 (d, J = 6.5 Hz, 3H). |
| 141 | ¹H NMR (500 MHz, DMSO-d₆) δ 8.51 (s, 1H), 7.97 (d, J = 9.8 Hz, 1H), 7.90 (d, |
| | J = 7.3 Hz, 1H), 7.67 (d, J = 8.6 Hz, 2H), 7.28 (t, J = 7.6 Hz, 1H), 7.18 (d, J = |
| | 7.6 Hz, 1H), 7.14 (d, J = 7.6 Hz, 1H), 5.23 (dd, J = 11.0, 3.6 Hz, 1H), 3.90-3.77 |
| | (m, 3H), 3.53-3.41 (m, 1H), 2.47-2.20 (m, 2H), 2.14-2.07 (m, 1H), 2.05 (s, |
| | 3H), 1.94 (s, 3H), 1.92-1.74 (m, 2H), 1.39 (s, 9H), 1.36-1.26 (m, 2H), 1.14 (d, |
| | J = 6.4 Hz, 3H). |
| 142 | ¹H NMR (500 MHz, DMSO-d₆) δ 8.56 (s, 1H), 7.93 (dd, J = 17.0, 8.5 Hz, 2H), |
| | 7.70 (s, 2H), 7.16 (dd, J = 5.1, 1.9 Hz, 3H), 7.08 (t, J = 7.6 Hz, 1H), 7.05-6.96 |
| | (m, 3H), 6.87 (d, J = 7.5 Hz, 1H), 5.19 (dd, J = 10.8, 3.6 Hz, 1H), 3.71 (t, J = |
| | 11.0 Hz, 1H), 3.52 (tt, J = 10.3, 3.8 Hz, 1H), 2.13 (s, 3H), 1.88 (s, 3H), 1.04 (d, |
| | J = 6.4 Hz, 3H). |
| 143 | ¹H NMR (500 MHz, DMSO-d₆) δ 8.56 (s, 1H), 8.51-8.46 (m, 1H), 8.34 (d, J = |
| | 2.2 Hz, 1H), 7.95 (dd, J = 11.2, 8.7 Hz, 2H), 7.76-7.67 (m, 3H), 7.48 (dd, J = |
| | 8.0, 5.1 Hz, 1H), 7.13 (t, J = 7.6 Hz, 1H), 7.02 (d, J = 7.7 Hz, 1H), 6.92 (d, J = |
| | 7.6 Hz, 1H), 5.21 (dd, J = 10.8, 3.6 Hz, 1H), 3.73 (t, J = 11.0 Hz, 1H), 3.51 (td, |
| | J = 11.1, 10.6, 5.1 Hz, 1H), 2.11 (s, 3H), 1.91 (s, 3H), 1.04 (d, J = 6.3 Hz, 3H). |
| 144 | ¹H NMR (500 MHz, DMSO-d₆) δ 8.55 (s, 1H), 7.90 (d, J = 9.8 Hz, 2H), 7.68 (s, |
| | 2H), 7.09 (d, J = 8.1 Hz, 1H), 7.01 (d, J = 7.6 Hz, 1H), 6.97-6.86 (m, 3H), 6.72 |
| | (d, J = 8.9 Hz, 2H), 5.18 (d, J = 10.4 Hz, 1H), 3.74-3.69 (m, 1H), 3.65 (s, 3H), |
| | 3.59-3.55 (m, 1H), 2.12 (s, 3H), 1.88 (s, 3H), 1.04 (d, J = 6.4 Hz, 3H). |
| 145 | ¹H NMR (500 MHz, DMSO-d₆) δ 8.62-8.53 (m, 3H), 8.01-7.91 (m, 2H), 7.78- |
| | 7.68 (m, 2H), 7.40 (d, J = 5.6 Hz, 2H), 7.15 (t, J = 7.6 Hz, 1H), 7.02 (d, J = 7.7 |
| | Hz, 1H), 6.93 (d, J = 7.6 Hz, 1H), 5.21 (dd, J = 10.6, 3.6 Hz, 1H), 3.73 (t, J = |
| | 11.0 Hz, 1H), 3.53 (ddt, J = 15.3, 10.5, 4.0 Hz, 1H), 2.10 (s, 3H), 1.92 (s, 3H), |
| | 1.05 (d, J = 6.3 Hz, 3H). |
| 146 | ¹H NMR (500 MHz, DMSO-d₆) δ 8.57 (s, 1H), 8.02 (d, J = 9.8 Hz, 1H), 7.94 (s, |
| | 1H), 7.70 (s, 2H), 7.41 (s, 1H), 7.26 (t, J = 7.6 Hz, 1H), 7.13 (d, J = 7.6 Hz, 1H), |
| | 7.07 (d, J = 7.6 Hz, 1H), 6.68 (s, 1H), 5.24 (d, J = 10.5 Hz, 1H), 3.93 (t, J = 11.0 |
| | Hz, 1H), 3.69 (s, 3H), 3.60-3.40 (m, 1H), 2.01 (s, 3H), 1.86 (s, 3H), 1.10 (d, J = |
| | 6.4 Hz, 3H). |
| 147 | ¹H NMR (400 MHz, DMSO-d₆) δ 12.30 (s, 1H), 8.54 (s, 1H), 8.08 (s, 1H), 7.94 |
| | (d, J = 7.1 Hz, 1H), 7.70 (s, 2H), 7.24 (s, 1H), 7.06 (s, 3H), 6.86 (s, 1H), 5.51 (s, |
| | 1H), 4.28 (s, 1H), 3.37 (s, 1H), 2.88 (s, 1H), 1.95 (s, 3H), 1.83 (s, 3H), 1.55 (d, |
| | J = 6.1 Hz, 3H), 1.22 (d, J = 6.6 Hz, 6H). |
| 148 | ¹H NMR (400 MHz, DMSO-d₆) δ 12.97 (s, 1H), 8.50 (s, 1H), 8.19 (s, 1H), 7.94 |
| | (s, 1H), 7.68 (s, 2H), 7.11 (s, 1H), 7.02-6.86 (m, 4H), 6.68 (d, J = 7.7 Hz, 2H), |
| | 5.43 (s, 1H), 3.64 (s, 3H), 3.38 (s, 1H), 2.76-2.70 (m, 1H), 1.96 (d, J = 41.9 Hz, |
| | 6H), 1.36 (s, 3H). |
| 149 | ¹H NMR (400 MHz, DMSO-d₆) δ 8.48 (s, 1H), 8.25 (s, 1H), 7.93 (s, 1H), 7.68 (s, |
| | 2H), 7.21 (s, 1H), 7.07 (d, J = 23.8 Hz, 2H), 5.41 (s, 1H), 5.24 (s, 1H), 3.54 (s, |
| | 3H), 3.18 (s, 2H), 2.76 (s, 1H), 2.12-1.87 (m, 8H), 1.47 (s, 3H), 1.33 (s, 9H). |
| 150 | ¹H NMR (400 MHz, Chloroform-d) δ 8.45 (s, 1H), 8.25 (s, 1H), 7.91 (s, 1H), 7.67 |
| | (s, 2H), 7.28 (s, 1H), 7.15 (d, J = 16.7 Hz, 2H), 5.45 (s, 1H), 3.86 (s, 4H), 2.76 (s, |
| | 1H), 2.04 (s, 4H), 1.91 (s, 4H), 1.49 (d, J = 6.1 Hz, 4H), 1.38 (s, 9H), 1.32 (d, J = |
| | 9.6 Hz, 2H), 1.24 (s, 2H). |
| 151 | ¹H NMR (400 MHz, DMSO-d₆) δ 12.65 (s, 1H), 8.48 (s, 1H), 8.11 (s, 1H), 7.91 |
| | (s, 1H), 7.66 (s, 2H), 7.25 (s, 1H), 7.12 (s, 2H), 5.50 (s, 1H), 3.77 (s, 4H), 2.94 (s, |
| | 1H), 2.03 (s, 6H), 1.89 (s, 2H), 1.56 (s, 4H), 1.30-1.13 (m, 2H). |
| 152 | ¹H NMR (400 MHz, DMSO-d₆) δ 12.36 (s, 1H), 8.49 (s, 1H), 7.98 (d, J = 48.7 Hz, |
| | 2H), 7.67 (s, 2H), 7.13 (d, J = 50.0 Hz, 3H), 5.36 (d, J = 43.2 Hz, 2H), 3.80 (s, |
| | 2H), 3.39 (d, J = 54.6 Hz, 3H), 2.80 (s, 1H), 1.99 (d, J = 52.9 Hz, 7H), 1.49 (s, 3H). |
| 153 | ¹H NMR (400 MHz, DMSO-d₆) δ 13.08 (s, 1H), 11.98 (s, 1H), 8.51 (s, 1H), 7.95 |
| | (s, 1H), 7.15 (s, 1H), 7.02 (s, 1H), 6.91 (s, 2H), 6.69 (s, 2H), 5.43 (s, 1H), 3.69 (s, |
| | 1H), 3.58 (s, 3H), 2.69 (s, 1H), 2.13 (d, J = 52.9 Hz, 3H), 1.98-1.63 (m, 3H), |
| | 1.29 (d, J = 21.3 Hz, 3H). |

### Example 50: Preparation of Compound 156

### Step 1: tert-Butyl 2-[[(1R)-4,4,4-trifluoro-1-(hydroxymethyl)-3,3-dimethyl-butyl]amino]-7-azaspiro[3.5]nonane-7-carboxylate

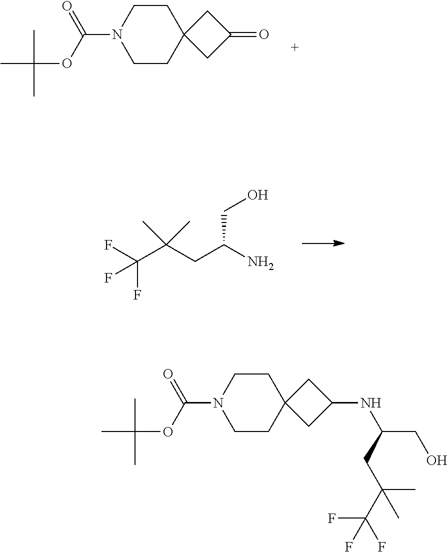 [SMILES: CC(C)(C)OC(=O)N1CCC2(CC1)CC(=O)C2 ; CC(C)(C[C@@H](N)CO)C(F)(F)F ; CC(C)(C)OC(=O)N1CCC2(CC1)CC(N[C@@H](CO)CC(C)(C)C(F)(F)F)C2]

In a 250 mL flask, (2R)-2-amino-5,5,5-trifluoro-4,4-dimethyl-pentan-1-ol (hydrochloride salt) (1.513 g, 6.826 mmol) was added to a solution of tert-butyl 2-oxo-7-azaspiro[3.5]nonane-7-carboxylate (1.496 g, 6.251 mmol) in anhydrous DCE (10 mL) under nitrogen and stirred at rt for 20 minutes (cloudy solution). sodium triacetoxyborohydride (3.98 g, 18.78 mmol) was divided into 3 separate portions and added to give a thick suspension (magnetic stirring still efficient). The mixture was stirred at rt for 22 hours. The mixture was cooled in an ice bath (internal temp=2° C.), then HCl (10 mL of 4 M, 40.00 mmol) was added very slowly, keeping the temperature between 2° C. and 6° C. A solution of potassium carbonate (10 g, 72.36 mmol) in water (10 mL) was added, while keeping the temperature below 10° C., then another portion of water (15 mL) (final pH=11) was added, followed by DCM (20 mL). The two phases were separated. The aqueous phase was further extracted with DCM (30 mL). The combined extracts were washed with saturated sodium bicarbonate (30 mL), dried over sodium sulfate and the solvents were evaporated. Drying under vacuum gave crude tert-butyl 2-[[(1R)-4,4,4-trifluoro-1-(hydroxymethyl)-3,3-dimethyl-butyl]amino]-7-azaspiro[3.5]nonane-7-carboxylate (2.446 g, 96%) as a brown honey-like resin. ESI-MS m/z calc. 408.25998, found 409.26 (M+1)⁺; Retention time: 1.32 minutes; LC method A. ¹H NMR (400 MHz, DMSO-d₆) δ 4.54 (t, J=5.5 Hz, 1H), 3.90 (s, 2H), 3.31-3.13 (m, 7H), 2.10-2.02 (m, 1H), 1.63-1.51 (m, 2H), 1.47-1.27 (m, 15H), 1.19-1.12 (m, 3H), 1.12-1.00 (m, 3H).

### Step 2: 3-[[4-[(2R)-2-[(7-tert-Butoxycarbonyl-7-azaspiro[3.5]nonan-2-yl)amino]-5,5,5-trifluoro-4,4-dimethyl-pentoxy]-6-(2,6-dimethylphenyl)-5-methyl-pyrimidin-2-yl]sulfamoyl]benzoic acid

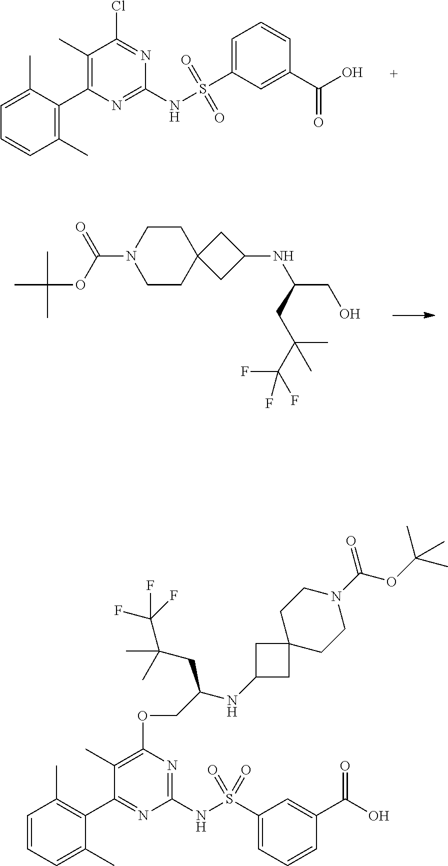 [SMILES: Cc1cccc(C)c1-c1nc(NS(=O)(=O)c2cccc(C(=O)O)c2)nc(Cl)c1C ; CC(C)(C)OC(=O)N1CCC2(CC1)CC(N[C@@H](CO)CC(C)(C)C(F)(F)F)C2 ; Cc1cccc(C)c1-c1nc(NS(=O)(=O)c2cccc(C(=O)O)c2)nc(OC[C@@H](CC(C)(C)C(F)(F)F)NC2CC3(CCN(C(=O)OC(C)(C)C)CC3)C2)c1C]

In a 20 mL vial, 3-[[4-chloro-6-(2,6-dimethylphenyl)-5-methyl-pyrimidin-2-yl]sulfamoyl]benzoic acid (430 mg, 0.9956 mmol) and tert-butyl 2-[[(1R)-4,4,4-trifluoro-1-(hydroxymethyl)-3,3-dimethyl-butyl]amino]-7-azaspiro[3.5]nonane-7-carboxylate (412 mg, 1.009 mmol) were charged under nitrogen with anhydrous THF (4 mL). Sodium tert-butoxide (493 mg, 5.130 mmol) was added (slight exotherm). The reaction was stirred at room temperature for 6 hours. The mixture was partitioned between ethyl acetate (50 mL) and aqueous 1M HCl (40 mL) and brine (20 mL). After separation, the aqueous phase was further extracted with EtOAc (2×30 mL). The combined extracts were dried over sodium sulfate and the solvents evaporated to give a solid. The solid was dissolved in DMSO (6 mL). The solution was microfiltered through a syringe filter disc and purified by reverse phase preparative HPLC (Cis) using a gradient of acetonitrile in water (1 to 99% over 15 min) and HCl as a modifier. The pure fractions were collected, a bit of brine was added, and the organic solvents were evaporated. The product was extracted with EtOAc (3×20 mL). After drying over sodium sulfate and evaporation, the residue was triturated with dichloromethane/hexanes. Evaporation of the solvents provided 3-[[4-[(2R)-2-[(7-tert-butoxycarbonyl-7-azaspiro[3.5]nonan-2-yl)amino]-5,5,5-trifluoro-4,4-dimethyl-pentoxy]-6-(2,6-dimethylphenyl)-5-methyl-pyrimidin-2-yl]sulfamoyl]benzoic acid (hydrochloride salt) (390 mg, 47%) as an off-white solid. ESI-MS m/z calc. 803.35394, found 804.98 (M+1)⁺; Retention time: 1.55 minutes (LC method A).

### Step 3: tert-Butyl 2-[(11R)-6-(2,6-dimethylphenyl)-7-methyl-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]-7-azaspiro[3.5]nonane-7-carboxylate (Compound 156)

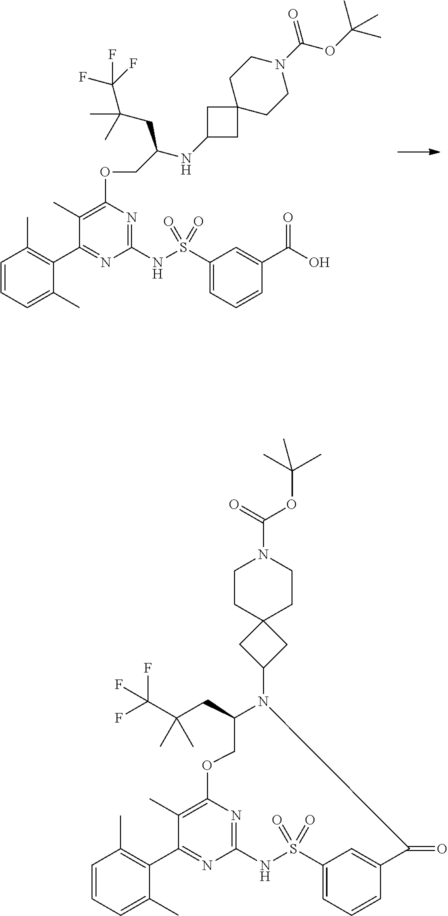 [SMILES: Cc1cccc(C)c1-c1nc(NS(=O)(=O)c2cccc(C(=O)O)c2)nc(OC[C@@H](CC(C)(C)C(F)(F)F)NC2CC3(CCN(C(=O)OC(C)(C)C)CC3)C2)c1C ; Cc1cccc(C)c1-c1nc2nc(c1C)OC[C@@H](CC(C)(C)C(F)(F)F)N(C1CC3(CCN(C(=O)OC(C)(C)C)CC3)C1)C(=O)c1cccc(c1)S(=O)(=O)N2]

A 250 mL flask was charged under nitrogen with HATU (367 mg, 0.9652 mmol), anhydrous DMF (10 mL) and DIEA (0.41 mL, 2.354 mmol). A solution of 3-[[4-[(2R)-2-[(7-tert-butoxycarbonyl-7-azaspiro[3.5]nonan-2-yl)amino]-5,5,5-trifluoro-4,4-dimethyl-pentoxy]-6-(2,6-dimethylphenyl)-5-methyl-pyrimidin-2-yl]sulfamoyl]benzoic acid (hydrochloride salt) (390 mg, 0.4641 mmol) in anhydrous DMF (10 mL) was added dropwise through syringe over a a period of 5 minutes. The mixture was stirred at room temperature for 29 hours. The mixture was concentrated and the residue was dissolved in DMSO (2 mL). The solution was microfiltered through a syringe filter disc and purified by reverse phase preparative HPLC (Cis) using a gradient of acetonitrile in water (1 to 99% over 15 min) and HCl as a modifier. The pure fractions were collected, a bit of brine and saturated sodium bicarbonate was added and the organic solvents were evaporated. The product was extracted with EtOAc (3×20 mL). After drying over sodium sulfate and evaporation, the residue was triturated with dichloromethane/hexanes. Evaporation of the solvents provided tert-butyl 2-[(11R)-6-(2,6-dimethylphenyl)-7-methyl-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]-7-azaspiro[3.5]nonane-7-carboxylate (79 mg, 22%) as a white solid. ESI-MS m/z calc. 785.3434, found 786.83 (M+1)⁺; Retention time: 2.01 minutes; LC method A.

### Example 51: Preparation of Compound 157

### Step 1: (11R)-12-{7-Azaspiro[3.5]nonan-2-yl}-6-(2,6-dimethylphenyl)-7-methyl-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (Compound 157)

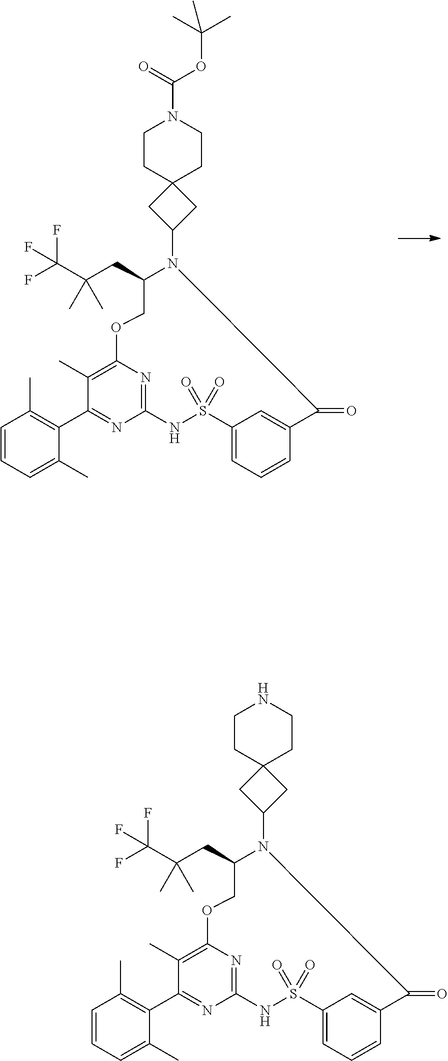 [SMILES: Cc1cccc(C)c1-c1nc2nc(c1C)OC[C@@H](CC(C)(C)C(F)(F)F)N(C1CC3(CCN(C(=O)OC(C)(C)C)CC3)C1)C(=O)c1cccc(c1)S(=O)(=O)N2 ; Cc1cccc(C)c1-c1nc2nc(c1C)OC[C@@H](CC(C)(C)C(F)(F)F)N(C1CC3(CCNCC3)C1)C(=O)c1cccc(c1)S(=O)(=O)N2]

A 100 mL flask containing tert-butyl 2-[(11R)-6-(2,6-dimethylphenyl)-7-methyl-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]-7-azaspiro[3.5]nonane-7-carboxylate (74 mg, 0.09416 mmol) was treated with DCM (600 μL) and HCl (300 μL of 4 M, 1.200 mmol) (4M in dioxane) at room temperature for 4 hours. The volatiles were removed by evaporation. DCM and hexanes were added and the solvents were evaporated. The operation was repeated until a white solid was obtained. Drying under vacuum gave (11R)-12-{7-azaspiro[3.5]nonan-2-yl}-6-(2,6-dimethylphenyl)-7-methyl-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt) (70 mg, 98%) as a white solid. ESI-MS m/z calc. 685.29095, found 686.78 (M+1)⁺; Retention time: 1.34 minutes; LC method A.

### Example 52: Preparation of Compound 158

### Step 1: tert-butyl N-tert-butoxycarbonyl-N-(4,6-dichloro-5-ethyl-pyrimidin-2-yl)carbamate

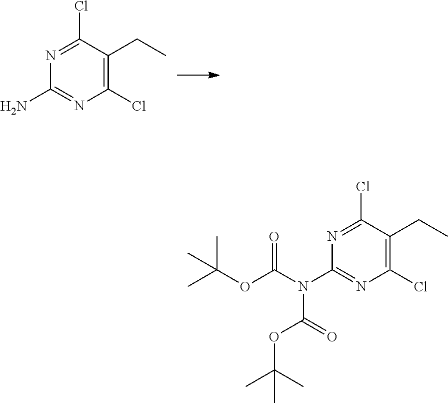 [SMILES: CCc1c(Cl)nc(N)nc1Cl ; CCc1c(Cl)nc(N(C(=O)OC(C)(C)C)C(=O)OC(C)(C)C)nc1Cl]

4,6-dichloro-5-ethyl-pyrimidin-2-amine (973 mg, 5.066 mmol) and Boc anhydride (2.36 g, 10.81 mmol) were dissolved in DCM (7.5 mL), followed by DMAP (50.5 mg, 0.4134 mmol). The reaction mixture was stirred at room temperature for 16 h. The reaction mixture was diluted with DCM (25 mL) and washed with water, then brine. The organics were dried over sodium sulfate and evaporated to give tert-butyl N-tert-butoxycarbonyl-N-(4,6-dichloro-5-ethyl-pyrimidin-2-yl)carbamate (1.94 g, 98%). ESI-MS m/z calc. 391.10657, found 392.1 (M+1)⁺; Retention time: 0.84 minutes; LC method D. ¹H NMR (400 MHz, Chloroform-d) δ 2.92 (q, J=7.5 Hz, 2H), 1.48 (s, 18H), 1.23 (t, J=7.5 Hz, 3H).

### Step 2: tert-Butyl N-tert-butoxycarbonyl-N-[4-chloro-6-(2,6-dimethylphenyl)-5-ethyl-pyrimidin-2-yl]carbamate

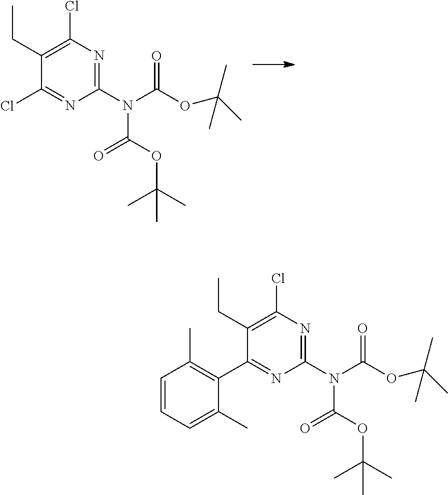 [SMILES: CCc1c(Cl)nc(N(C(=O)OC(C)(C)C)C(=O)OC(C)(C)C)nc1Cl ; CCc1c(Cl)nc(N(C(=O)OC(C)(C)C)C(=O)OC(C)(C)C)nc1-c1c(C)cccc1C]

To a solution of tert-butyl N-tert-butoxycarbonyl-N-(4,6-dichloro-5-ethyl-pyrimidin-2-yl)carbamate (34.92 g, 89.019 mmol) dissolved in DME (250 mL) and water (50 mL) was added (2,6-dimethylphenyl)boronic acid (13.35 g, 89.010 mmol) and cesium carbonate (75.4 g, 231.42 mmol) at room temperature. The solution was stirred for 10 min while being bubbled with a nitrogen stream. Then Pd(dppf)Cl₂ (5.2 g, 7.1067 mmol) was added to the solution and heated to 80° C. overnight. The solution was cooled to room temperature before being diluted with water (250 mL) and extracted with ethyl acetate (2×300 mL). The combined organic layer was washed with brine (400 mL) and dried over sodium sulfate before being concentrated in vacuum to give tert-butyl N-tert-butoxycarbonyl-N-[4-chloro-6-(2,6-dimethylphenyl)-5-ethyl-pyrimidin-2-yl]carbamate (37.51 g, 43%). ESI-MS m/z calc. 461.20813, found 462.2 (M+1)⁺; Retention time: 3.85 minutes; LC method T.

### Step 3: 4-Chloro-6-(2,6-dimethylphenyl)-5-ethyl-pyrimidin-2-amine

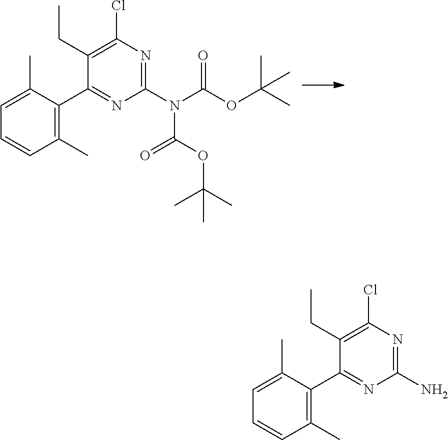 [SMILES: CCc1c(Cl)nc(N(C(=O)OC(C)(C)C)C(=O)OC(C)(C)C)nc1-c1c(C)cccc1C ; CCc1c(Cl)nc(N)nc1-c1c(C)cccc1C]

tert-Butyl N-tert-butoxycarbonyl-N-[4-chloro-6-(2,6-dimethylphenyl)-5-ethyl-pyrimidin-2-yl]carbamate (393 mg, 0.8507 mmol) and HCl in dioxane (3 mL of 4 M, 12.00 mmol) were combined in dichloromethane (4 mL) and stirred for 16 h. The reaction mixture was evaporated to dryness. The resulting material was partitioned between ethyl acetate and a saturated sodium bicarbonate solution and the mixture was stirred for 15 min. The organics were separated, washed with brine, dried over sodium sulfate and evaporated to give 4-chloro-6-(2,6-dimethylphenyl)-5-ethyl-pyrimidin-2-amine (219 mg, 98%). ESI-MS m/z calc. 261.10327, found 262.1 (M+1)⁺; Retention time: 0.64 minutes; LC method D.

### Step 4: Methyl 3-[[4-chloro-6-(2,6-dimethylphenyl)-5-ethyl-pyrimidin-2-yl]sulfamoyl]benzoate

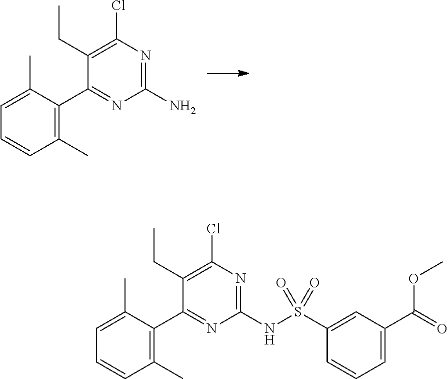 [SMILES: CCc1c(Cl)nc(N)nc1-c1c(C)cccc1C ; CCc1c(Cl)nc(NS(=O)(=O)c2cccc(C(=O)OC)c2)nc1-c1c(C)cccc1C]

4-Chloro-6-(2,6-dimethylphenyl)-5-ethyl-pyrimidin-2-amine (219 mg, 0.8367 mmol) was dissolved in THF (2.2 mL) and cooled in an ice bath. methyl 3-chlorosulfonylbenzoate (607.2 mg, 2.588 mmol) was added in one portion. lithium tert-amoxide (1.5 mL of 40% w/w, 4.656 mmol) was added dropwise and the reaction was allowed to slowly warm to room temperature. The reaction was stirred for 5 h, then made acidic by the addition of 1M HCl. The reaction mixture was extracted with ethyl acetate. The organics were washed with brine, dried over sodium sulfate and evaporated. The crude material was purified by silica gel chromatography eluting with 0-50% ethyl acetate in hexanes to give methyl 3-[[4-chloro-6-(2,6-dimethylphenyl)-5-ethyl-pyrimidin-2-yl]sulfamoyl]benzoate (294 mg, 76%). ESI-MS m/z calc. 459.10196, found 460.1 (M+1)⁺; Retention time: 0.77 minutes; (LC method D).

### Step 5: 3-[[4-Chloro-6-(2,6-dimethylphenyl)-5-ethyl-pyrimidin-2-yl]sulfamoyl]benzoic acid

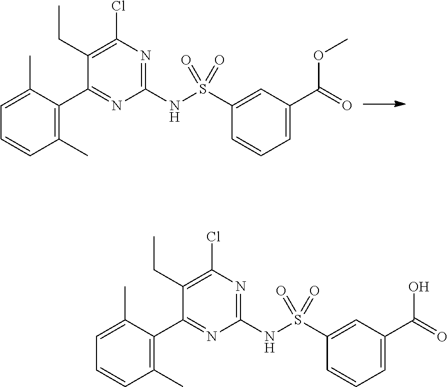 [SMILES: CCc1c(Cl)nc(NS(=O)(=O)c2cccc(C(=O)OC)c2)nc1-c1c(C)cccc1C ; CCc1c(Cl)nc(NS(=O)(=O)c2cccc(C(=O)O)c2)nc1-c1c(C)cccc1C]

To a solution of methyl 3-[[4-chloro-6-(2,6-dimethylphenyl)-5-ethyl-pyrimidin-2-yl]sulfamoyl]benzoate (4.75 g, 10.327 mmol) in THF (100 mL) was added an aqueous solution of NaOH (45 mL of 1 M, 45.000 mmol) and stirred for 1 hour at room temperature. The solution was acidified using 1M HCl (75 mL) and extracted with ethyl acetate (2×100 mL) before being washed with brine (100 mL). The organic layer was dried over sodium sulfate and concentrated in vacuum to give 3-[[4-chloro-6-(2,6-dimethylphenyl)-5-ethyl-pyrimidin-2-yl]sulfamoyl]benzoic acid (3.83 g, 78%) as a white solid. ESI-MS m/z calc. 445.0863, found 446.2 (M+1)⁺; Retention time: 2.52 minutes; LC method T. ¹H NMR (500 MHz, DMSO-d₆) δ 13.39 (s, 1H), 12.28 (s, 1H), 8.41 (s, 1H), 8.17 (dt, J=7.8, 1.4 Hz, 1H), 8.09 (dt, J=7.9, 1.5 Hz, 1H), 7.66 (t, J=7.8 Hz, 1H), 7.26 (t, J=7.6 Hz, 1H), 7.13 (d, J=7.6 Hz, 2H), 2.26 (q, J=7.5 Hz, 2H), 1.75 (s, 6H), 0.83 (t, J=7.5 Hz, 3H).

### Step 6: tert-butyl 2-[(11R)-6-(2,6-dimethylphenyl)-7-ethyl-11-isobutyl-2,2,13-trioxo-9-oxa-2λ⁶-thia-3,5,12,19-tetrazatricyclo[12.3.1.14,8]nonadeca-1(17),4(19),5,7,14(18),15-hexaen-12-yl]-7-azaspiro[3.5]nonane-7-carboxylate, (Compound 158), and (11R)-12-(7-azaspiro[3.5]nonan-2-yl)-6-(2,6-dimethylphenyl)-7-ethyl-11-isobutyl-2,2-dioxo-9-oxa-2λ⁶-thia-3,5,12,19-tetrazatricyclo[12.3.1.14,8]nonadeca-1(17),4(19),5,7,14(18),15-hexaen-13-one

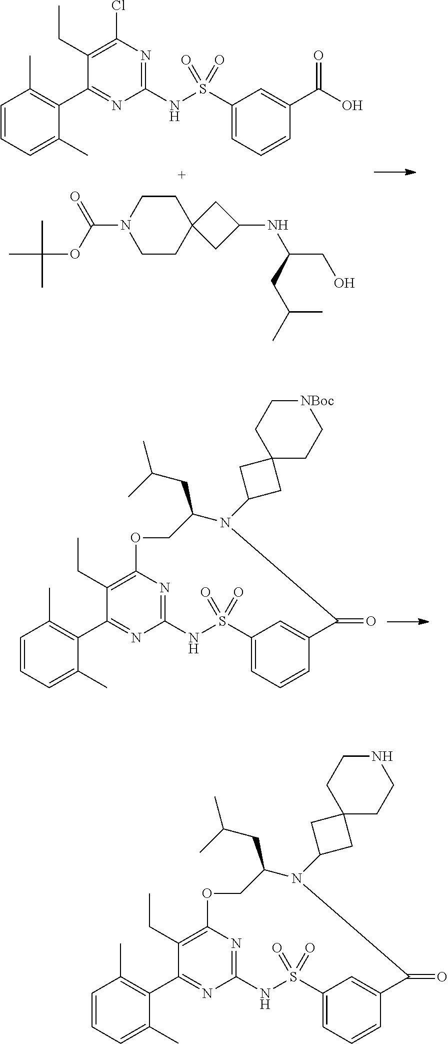

3-[[4-chloro-6-(2,6-dimethylphenyl)-5-ethyl-pyrimidin-2-yl]sulfamoyl]benzoic acid (50 mg, 0.1121 mmol), tert-butyl 2-[[(1R)-1-(hydroxymethyl)-3-methyl-butyl]amino]-7-azaspiro[3.5]nonane-7-carboxylate (hydrochloride salt) (43 mg, 0.1141 mmol), and sodium tert-butoxide (55 mg, 0.5723 mmol) were combined in THF (350 μL) (slight exotherm). The reaction was heated at 40° C. for 30 min then cooled to room temperature and added dropwise to a stirring solution of [dimethylamino(triazolo[4,5-b]pyridin-3-yloxy)methylene]-dimethyl-ammonium;hexafluorophosphate (8,585.25 mg, 22.58 mmol) in DMF (1.5 mL). The reaction mixture was allowed to stir at room temperature for 1 hour 15 minutes, then DIPEA (50 μL, 0.2871 mmol) was added. After an additional 15 minutes at room temperature, the reaction mixture was filtered and purified by reverse phase HPLC (1-99 ACN in water, HCl modifier, 15 min run) and the fractions containing product were dried by rotary evaporation to give tert-butyl 2-[(11R)-6-(2,6-dimethylphenyl)-7-ethyl-11-isobutyl-2,2,13-trioxo-9-oxa-2λ⁶-thia-3,5,12,19-tetrazatricyclo[12.3.1.14,8]nonadeca-1(17),4(19),5,7,14(18),15-hexaen-12-yl]-7-azaspiro[3.5]nonane-7-carboxylate (21 mg, 25%). ESI-MS m/z calc. 731.37164, found 732.6 (M+1)⁺; Retention time: 2.24 minutes; (LC method A). A 14 mg portion of the product was dissolved in dichloromethane (500 μL) and HCl (500 μL of 4 M, 2.000 mmol) in dioxane was added by syringe. The reaction mixture was stirred at room temperature for 20 minutes then the volatiles were removed. Hexane was added and the material was again concentrated to give a slightly yellow solid. (11R)-12-(7-azaspiro[3.5]nonan-2-yl)-6-(2,6-dimethylphenyl)-7-ethyl-11-isobutyl-2,2-dioxo-9-oxa-2λ⁶-thia-3,5,12,19-tetrazatricyclo[12.3.1.14,8]nonadeca-1(17),4(19),5,7,14(18),15-hexaen-13-one (hydrochloride salt) (12 mg, 16%) ESI-MS m/z calc. 631.3192, found 632.6 (M+1)⁺; Retention time: 0.55 minutes; (LC method D).

### Example 53: Preparation of Compound 159

### Step 1: (11R)-6-(2,6-Dimethylphenyl)-7-ethyl-11-isobutyl-12-(7-methyl-7-azaspiro[3.5]nonan-2-yl)-2,2-dioxo-9-oxa-2λ⁶-thia-3,5,12,19-tetrazatricyclo[12.3.1.14,8]nonadeca-1(17),4(19),5,7,14(18),15-hexaen-13-one (Compound 159)

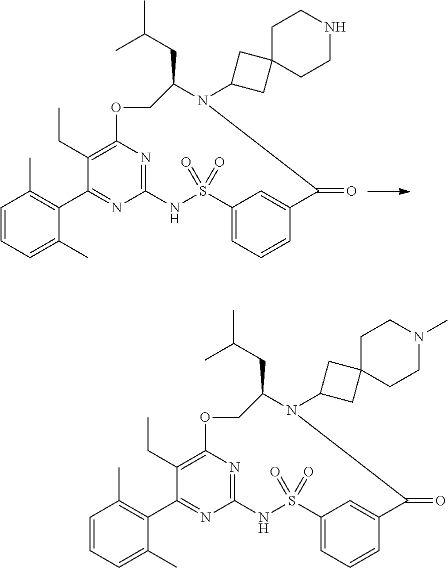 [SMILES: CCc1c2nc(nc1-c1c(C)cccc1C)NS(=O)(=O)c1cccc(c1)C(=O)N(C1CC3(CCNCC3)C1)[C@H](CC(C)C)CO2 ; CCc1c2nc(nc1-c1c(C)cccc1C)NS(=O)(=O)c1cccc(c1)C(=O)N(C1CC3(CCN(C)CC3)C1)[C@H](CC(C)C)CO2]

(11R)-12-(7-azaspiro[3.5]nonan-2-yl)-6-(2,6-dimethylphenyl)-7-ethyl-11-isobutyl-2,2-dioxo-9-oxa-2λ⁶-thia-3,5,12,19-tetrazatricyclo[12.3.1.14,8]nonadeca-1(17),4(19),5,7,14(18),15-hexaen-13-one (hydrochloride salt) (12 mg, 0.01796 mmol) was dissolved in formic acid (150 μL) and combined with aqueous formaldehyde (350 μL, 12.71 mmol) and heated to 95° C. for 16 hours in a screwcap vial. The reaction mixture was then partially concentrated under reduced pressure, diluted with methanol, filtered, then purified by reverse phase HPLC (1-70% ACN in water, HCl modifier, 15 minutes). The fractions containing product were combined and concentrated to give after drying as a white powder, (11R)-6-(2,6-dimethylphenyl)-7-ethyl-11-isobutyl-12-(7-methyl-7-azaspiro[3.5]nonan-2-yl)-2,2-dioxo-9-oxa-2λ⁶-thia-3,5,12,19-tetrazatricyclo[12.3.1.14,8]nonadeca-1(17),4(19),5,7,14(18),15-hexaen-13-one (hydrochloride salt) (6.9 mg, 56%). ESI-MS m/z calc. 645.3349, found 646.6 (M+1)⁺; Retention time: 1.37 minutes; LC method A.

### Example 54: Preparation of Compound 160

### Step 1: 3-[[4-(2,6-Dimethylphenyl)-5-ethyl-6-[(2R)-4-methyl-2-(spiro[2.3]hexan-5-ylamino)pentoxy]pyrimidin-2-yl]sulfamoyl]benzoic acid

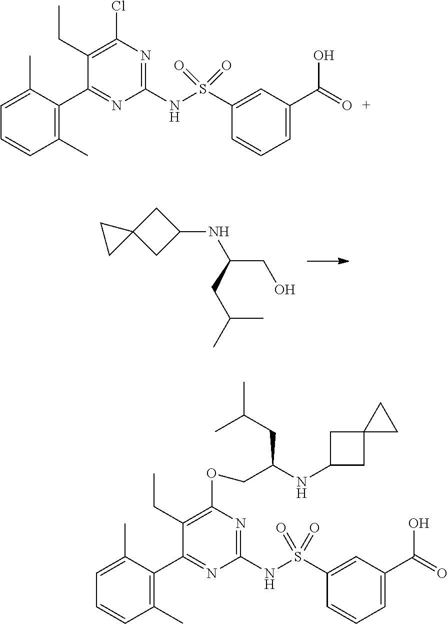

3-[[4-chloro-6-(2,6-dimethylphenyl)-5-ethyl-pyrimidin-2-yl]sulfamoyl]benzoic acid (56.1 mg, 0.1258 mmol), (2R)-4-methyl-2-(spiro[2.3]hexan-5-ylamino)pentan-1-ol (hydrochloride salt) (34.7 mg, 0.1484 mmol), and sodium tert-butoxide (58 mg, 0.6035 mmol) were combined in THF (1 mL) and heated at 45° C. for 3 h. The reaction was partitioned between ethyl acetate and a 1M HCl solution. The organics were separated, washed with brine, dried over sodium sulfate and evaporated. The crude material was purified by reverse phase HPLC utilizing a gradient of 1-99% acetonitrile in 5 mM aqueous HCl to yield 3-[[4-(2,6-dimethylphenyl)-5-ethyl-6-[(2R)-4-methyl-2-(spiro[2.3]hexan-5-ylamino)pentoxy]pyrimidin-2-yl]sulfamoyl]benzoic acid (hydrochloride salt) (23.0 mg, 25%). ESI-MS m/z calc. 606.2876, found 607.4 (M+1)⁺; Retention time: 0.55 minutes; LC method D.

### Step 2: (11R)-6-(2,6-Dimethylphenyl)-7-ethyl-11-isobutyl-2,2-dioxo-12-spiro[2.3]hexan-5-yl-9-oxa-2λ⁶-thia-3,5,12,19-tetrazatricyclo[12.3.1.14,8]nonadeca-1(17),4(19),5,7,14(18),15-hexaen-13-one (Compound 160)

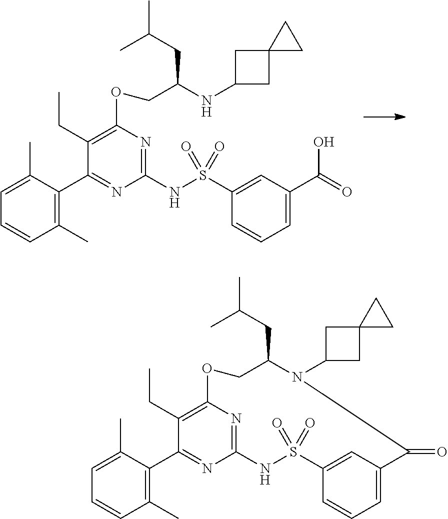

3-[[4-(2,6-Dimethylphenyl)-5-ethyl-6-[(2R)-4-methyl-2-(spiro[2.3]hexan-5-ylamino)pentoxy]pyrimidin-2-yl]sulfamoyl]benzoic acid (hydrochloride salt) (23 mg, 0.03111 mmol) was dissolved in DMF (1 mL). HATU (16.5 mg, 0.04339 mmol), followed by triethylamine (17 μL, 0.1220 mmol) were added and the reaction mixture was stirred for 3 h. The reaction was filtered and purified by reverse phase HPLC utilizing a gradient of 1-99% acetonitrile in 5 mM aqueous HCl to yield (11R)-6-(2,6-dimethylphenyl)-7-ethyl-11-isobutyl-2,2-dioxo-12-spiro[2.3]hexan-5-yl-9-oxa-2λ⁶-thia-3,5,12,19-tetrazatricyclo[12.3.1.14,8]nonadeca-1(17),4(19),5,7,14(18),15-hexaen-13-one (11 mg, 60%). ESI-MS m/z calc. 588.27704, found 589.4 (M+1)⁺; Retention time: 2.15 minutes; LC method A.

### Example 55: Preparation of Compound 161

### Step 1: 3-[[4-[(2R)-2-amino-4,4-dimethyl-pentoxy]-6-(2,6-dimethylphenyl)-5-ethyl-pyrimidin-2-yl]sulfamoyl]benzoic acid

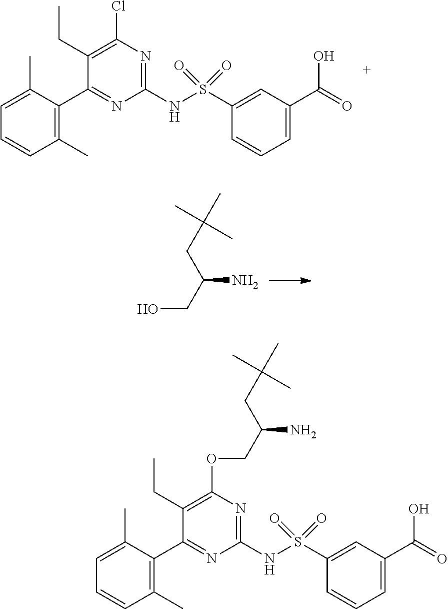

A solution of 3-[[4-chloro-6-(2,6-dimethylphenyl)-5-ethyl-pyrimidin-2-yl]sulfamoyl]benzoic acid (1.2 g, 2.691 mmol), (2R)-2-amino-4,4-dimethyl-pentan-1-ol (0.36 g, 2.744 mmol), and sodium tert-butoxide (0.78 g, 8.116 mmol) in THF (13 mL) was stirred for 18 hours. UPLCMS showed about 50% conversion to product, so (2R)-2-amino-4,4-dimethyl-pentan-1-ol (0.18 g, 1.372 mmol) and sodium tert-butoxide (0.28 g, 2.914 mmol) were added, and the reaction was stirred for a day. The reaction was acidified with 1 M citric acid, diluted with water, and extracted with ethyl acetate. The combined extracts were washed with brine, dried over sodium sulfate, and evaporated under vacuum. The residue was purified by silica gel column chromatography with 0-10% methanol in dichloromethane to give 3-[[4-[(2R)-2-amino-4,4-dimethyl-pentoxy]-6-(2,6-dimethylphenyl)-5-ethyl-pyrimidin-2-yl]sulfamoyl]benzoic acid (0.70 g, 48%) ESI-MS m/z calc. 540.24066, found 541.3 (M+1)⁺; Retention time: 0.49 minutes as a colorless solid. ESI-MS m/z calc. 540.24066, found 541.3 (M+1)⁺; Retention time: 0.49 minutes; LC method D.

### Step 2: (11R)-6-(2,6-Dimethylphenyl)-11-(2,2-dimethylpropyl)-7-ethyl-9-oxa-2λ⁶-thia-3,5,12,19-tetraazatricyclo[12.3.1.14,8]nonadeca-1(18),4(19),5,7,14,16-hexaene-2,2,13-trione (Compound 161)

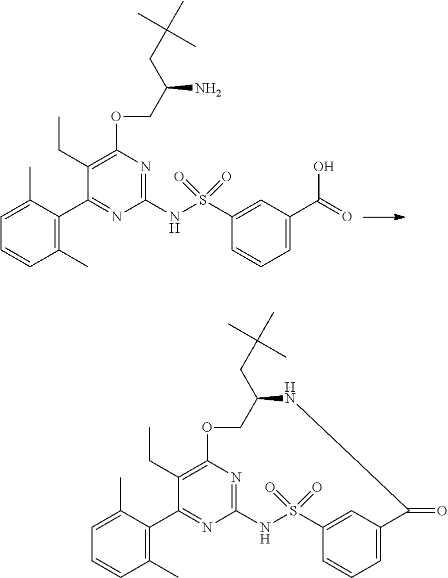 [SMILES: CCc1c(OC[C@H](N)CC(C)(C)C)nc(NS(=O)(=O)c2cccc(C(=O)O)c2)nc1-c1c(C)cccc1C ; CCc1c2nc(nc1-c1c(C)cccc1C)NS(=O)(=O)c1cccc(c1)C(=O)N[C@H](CC(C)(C)C)CO2]

A solution of 3-[[4-[(2R)-2-amino-4,4-dimethyl-pentoxy]-6-(2,6-dimethylphenyl)-5-ethyl-pyrimidin-2-yl]sulfamoyl]benzoic acid (55 mg, 0.1017 mmol), [[(E)-(1-cyano-2-ethoxy-2-oxo-ethylidene)amino]oxy-tetrahydropyran-4-yl-methylene]-dimethyl-ammonium (Phosphorus Hexafluoride Ion) (67 mg, 0.1568 mmol), and DIEA (54 μL, 0.3100 mmol) in DMF (5 mL) was stirred for 15 hours. The reaction was acidified with 1 M HCl, diluted with water, and extracted with ethyl acetate. The combined extracts were washed with brine and water, dried over sodium sulfate, and evaporated under vacuum. The residue was purified by reverse-phase HPLC-MS (1%-99% acetonitrile/water (5 mM HCl)) to give (11R)-6-(2,6-dimethylphenyl)-11-(2,2-dimethylpropyl)-7-ethyl-9-oxa-2λ⁶-thia-3,5,12,19-tetraazatricyclo[12.3.1.14,8]nonadeca-1(18),4(19),5,7,14,16-hexaene-2,2,13-trione (22.2 mg, 41%). ESI-MS m/z calc. 522.2301, found 523.3 (M+1)⁺; Retention time: 1.64 minutes; LC method A.

### Example 56: Preparation of Compound 162

### Step 1: 3-[[4-Chloro-6-(2,6-dimethylphenyl)-5-(trifluoromethyl)pyrimidin-2-yl]sulfamoyl]benzoic acid

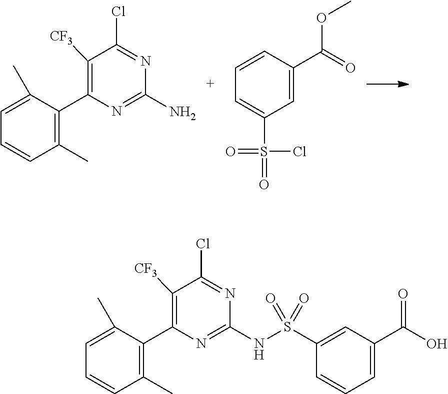

To an ice bath cooled solution of sodium hydride (62 mg of 60% w/w, 1.550 mmol) in DMF (3 mL) was added 4-chloro-6-(2,6-dimethylphenyl)-5-(trifluoromethyl)pyrimidin-2-amine (116.2 mg, 0.3852 mmol) and the mixture was stirred for 15 min. methyl 3-chlorosulfonylbenzoate (228 mg, 0.9716 mmol) was added and the reaction was allowed to warm to room temperature over 1 h. The reaction was partitioned between ethyl acetate and a 1M HCl solution. The organics were separated, washed with brine, dried over sodium sulfate and evaporated. The crude material was used directly in the next step. The product was dissolved in a mixture of THF (3 mL) and NaOH (3 mL of 1 M, 3.000 mmol) and stirred at room temperature for 30 min. The reaction was partitioned between ethyl acetate and a 1M HCl solution. The organics were separated, washed with brine, dried over sodium sulfate and evaporated. The crude product was purified by reverse phase HPLC utilizing a gradient of 1-99% acetonitrile in 5 mM aq HCl to yield 3-[[4-chloro-6-(2,6-dimethylphenyl)-5-(trifluoromethyl)pyrimidin-2-yl]sulfamoyl]benzoic acid (35 mg, 19%). ESI-MS m/z calc. 485.0424, found 486.0 (M+1)⁺; Retention time: 0.68 minutes, LC method D.

### Step 2: 3-[[4-(2,6-dimethylphenyl)-6-[(2R)-4-methyl-2-(spiro[2.3]hexan-5-ylamino)pentoxy]-5-(trifluoromethyl)pyrimidin-2-yl]sulfamoyl]benzoic acid

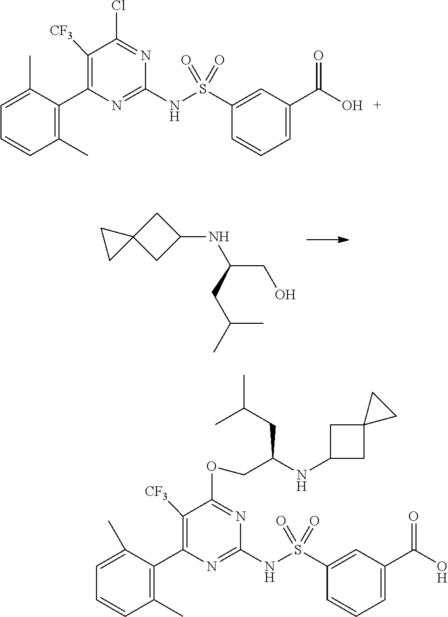

3-[[4-chloro-6-(2,6-dimethylphenyl)-5-(trifluoromethyl)pyrimidin-2-yl]sulfamoyl]benzoic acid (35 mg, 0.07204 mmol), (2R)-4-methyl-2-(spiro[2.3]hexan-5-ylamino)pentan-1-ol (hydrochloride salt) (24 mg, 0.1027 mmol), and sodium tert-butoxide (37.4 mg, 0.3892 mmol) were combined in THF (1 mL) and stirred at room temperature for 30 min. The reaction was partitioned between ethyl acetate and a 1M HCl solution. The organics were separated, washed with brine, dried over sodium sulfate and evaporated. The crude material was purified by reverse phase HPLC utilizing a gradient of 1-99% acetonitrile in 5 mM aqueous HCl to yield 3-[[4-(2,6-dimethylphenyl)-6-[(2R)-4-methyl-2-(spiro[2.3]hexan-5-ylamino)pentoxy]-5-(trifluoromethyl)pyrimidin-2-yl]sulfamoyl]benzoic acid (hydrochloride salt) (8 mg, 16%). ESI-MS m/z calc. 646.24365, found 647.3 (M+1)⁺; Retention time: 0.59 minutes; LC method D.

### Step 3: (11R)-6-(2,6-dimethylphenyl)-11-isobutyl-2,2-dioxo-12-spiro[2.3]hexan-5-yl-7-(trifluoromethyl)-9-oxa-2λ⁶-thia-3,5,12,19-tetrazatricyclo[12.3.1.14,8]nonadeca-1(18),4(19),5,7,14,16-hexaen-13-one (Compound 162)

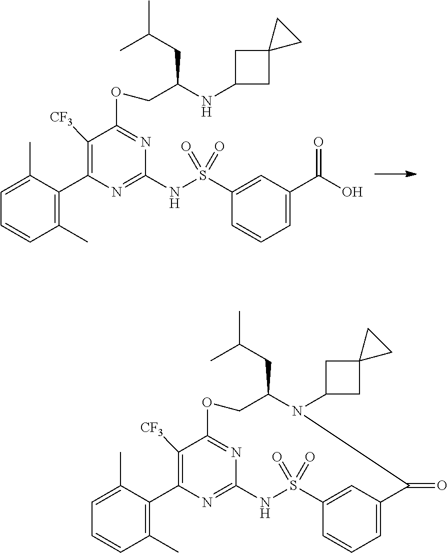 [SMILES: Cc1cccc(C)c1-c1nc(NS(=O)(=O)c2cccc(C(=O)O)c2)nc(OC[C@@H](CC(C)C)NC2CC3(CC3)C2)c1C(F)(F)F ; Cc1cccc(C)c1-c1nc2nc(c1C(F)(F)F)OC[C@@H](CC(C)C)N(C1CC3(CC3)C1)C(=O)c1cccc(c1)S(=O)(=O)N2]

3-[[4-(2,6-dimethylphenyl)-6-[(2R)-4-methyl-2-(spiro[2.3]hexan-5-ylamino)pentoxy]-5-(trifluoromethyl)pyrimidin-2-yl]sulfamoyl]benzoic acid (hydrochloride salt) (8 mg, 0.01171 mmol) was dissolved in DMF (1 mL), followed by HATU (6.5 mg, 0.01709 mmol), then triethylamine (8 μL, 0.05740 mmol). The reaction mixture was stirred at room temperature for 16 h. The reaction was filtered and purified by reverse phase HPLC utilizing a gradient of 1-99% acetonitrile in 5 mM aqueous HCl to yield (11R)-6-(2,6-dimethylphenyl)-11-isobutyl-2,2-dioxo-12-spiro[2.3]hexan-5-yl-7-(trifluoromethyl)-9-oxa-2λ⁶-thia-3,5,12,19-tetrazatricyclo[12.3.1.14,8]nonadeca-1(18),4(19),5,7,14,16-hexaen-13-one (5.1 mg, 69%). ESI-MS m/z calc. 628.2331, found 629.4 (M+1)⁺; Retention time: 2.24 minutes; LC method A.

### Example 57: Preparation of Compound 163

### Step 1: 2-Amino-6-(2,6-dimethylphenyl)pyrimidin-4-ol

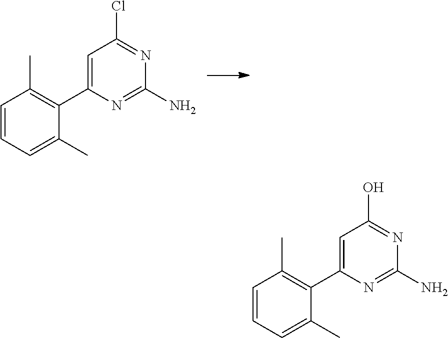 [SMILES: Cc1cccc(C)c1-c1cc(Cl)nc(N)n1 ; Cc1cccc(C)c1-c1cc(O)nc(N)n1]

A solution of 4-chloro-6-(2,6-dimethylphenyl)pyrimidin-2-amine (hydrochloride salt) (22.5 g, 83.285 mmol) in dioxane (225 mL) and aqueous sodium hydroxide (415 mL of 1 M, 415.00 mmol) was heated in an oil bath set at 80° C. for about 28 hours. Once cooled to room temperature, the crude reaction mixture was diluted with water (400 mL) and cooled in an ice bath. The mixture was acidified to pH of about 4 using concentrated hydrochloric. The reaction mixture was transferred to a 2 L separatory funnel and extracted with a mixture of isopropanol in chloroform (1:4; 4×300 mL). The combined organic layers were dried over sodium sulfate, filtered and concentrated under reduced pressure to afford 2-amino-6-(2,6-dimethylphenyl)pyrimidin-4-ol (17 g, 94%) as a pale brown solid. ESI-MS m/z calc. 215.10587, found 216.2 (M+1)⁺; Retention time: 1.39 minutes; LC method K.

### Step 2: 2-Amino-6-[2,6-dimethyl-3-(trifluoromethyl)phenyl]-5-(trifluoromethyl)pyrimidin-4-ol

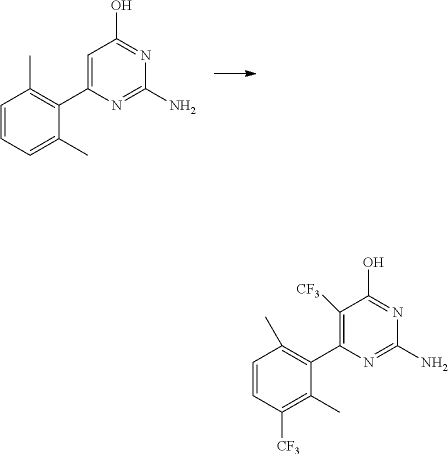 [SMILES: Cc1cccc(C)c1-c1cc(O)nc(N)n1 ; Cc1ccc(C(F)(F)F)c(C)c1-c1nc(N)nc(O)c1C(F)(F)F]

A solution of 2-amino-6-(2,6-dimethylphenyl)pyrimidin-4-ol (15.3 g, 71.080 mmol) in DMSO (140 mL) was treated with a solution of trifluoroiodomethane (39 g, 199.07 mmol) in DMSO (70 mL). Added bis(cyclopentadienyl)iron (4 g, 21.502 mmol), then hydrogen peroxide (35 mL of 35% aqueous solution, 360 mmol) and the reaction was left to stir at room temperature for 1 hours in a water bath to mitigate the exotherm. The crude was transferred to a 4.0-L separatory funnel with water (2.0 L) and extracted with ethyl acetate (4×400 mL). The combined organic layers were washed with water (2×400 mL), brine (300 mL), dried over sodium sulfate, filtered and concentrated under reduced pressure. The residue was split in two and purified by silica gel chromatography on two separate 330-g columns eluting from 0% to 10% methanol in dichloromethane to afford a roughly 1:1 mixture of 2-amino-6-(2,6-dimethylphenyl)-5-(trifluoromethyl)pyrimidin-4-ol and 2-amino-6-[2,6-dimethyl-3-(trifluoromethyl)phenyl]-5-(trifluoromethyl)pyrimidin-4-ol (12.22 g) as a brown solid. This residue was further purified by reverse-phase chromatography, eluting with mixtures of acetonitrile and water (both containing 0.1% formic acid) to afford 2-amino-6-(2,6-dimethylphenyl)-5-(trifluoromethyl)pyrimidin-4-ol (3.4 g, 17%) as an orange-pink solid, 1H NMR (300 MHz, DMSO-d₆) δ 11.52 (br. s., 1H), 7.14-7.07 (m, 1H), 7.05-6.98 (m, 2H), 2.05 (s, 6H). ¹⁹F NMR (282 MHz, DMSO-d₆) δ −56.60 (s, 3F). ESI-MS m/z calc. 283.0932, found 284.1 (M+1)⁺; Retention time: 1.69 minutes (LC method K); and 2-amino-6-[2,6-dimethyl-3-(trifluoromethyl)phenyl]-5-(trifluoromethyl)pyrimidin-4-ol (4.37 g, 17%) as a pale brown solid. ¹H NMR (300 MHz, DMSO-d₆) δ 11.64 (br. s., 1H), 7.56 (d, J=8.1 Hz, 1H), 7.27 (d, J=8.1 Hz, 1H), 2.19-2.15 (m, 3H), 2.13 (s, 3H). ¹⁹F NMR (282 MHz, DMSO-d₆) δ −56.73 (s, 3F), −59.42 (s, 3F). ESI-MS m/z calc. 351.0806, found 352.1 (M+1)⁺; Retention time: 1.86 minutes (LC method K). The latter product is a mixture of 2-amino-6-[2,6-dimethyl-3-(trifluoromethyl)phenyl]-5-(trifluoromethyl)pyrimidin-4-ol and 2-amino-6-[2,6-dimethyl-4-(trifluoromethyl)phenyl]-5-(trifluoromethyl)pyrimidin-4-ol in a roughly 6:1 ratio, according to NMR. Conditions for purification by reverse-phase: purified on an InnoFlash SW220 column (spherical C18, 20-45 m, 100 Å) with a flow rate of 50 mL/min. Elution conditions were 5% ACN in water (containing 1% formic acid) for 4 minutes, then 5% to 30% over 2 minutes, then 30% to 80% over 24 minutes, then 80% to 100% over 2 minutes and maintained at 100% ACN for 8 minutes. A total of 8 injections of about 1.5 g were performed. Fractions of 25 mL were collected. The desired product was typically found in fractions 42-50, and the bis-trifluoromethylated adduct in fractions 52-58.

### Step 3: N′-[4-Chloro-6-[2,6-dimethyl-3-(trifluoromethyl)phenyl]-5-(trifluoromethyl)pyrimidin-2-yl]-N,N-dimethyl-formamidine

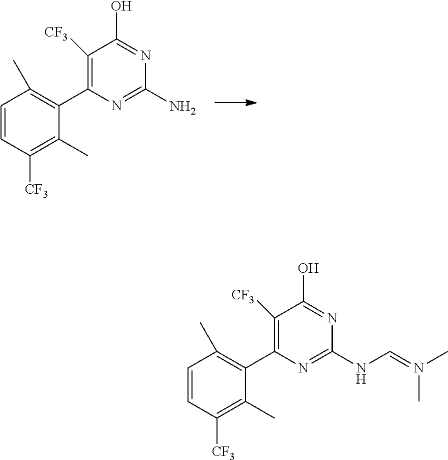

Oxalyl chloride (7.1295 g, 4.9 mL, 56.171 mmol) was slowly added (CAUTION: Carbon monoxide, use a detector) to a solution of dimethylformamide (4.1536 g, 4.4 mL, 56.826 mmol) in chloroform (190 mL) in a three neck flask (0.25 L) and the solution was stirred at room temperature for 30 minutes. The solution became light yellow. 2-Amino-6-[2,6-dimethyl-3-(trifluoromethyl)phenyl]-5-(trifluoromethyl)pyrimidin-4-ol (6:1 mixture with 2-amino-6-[2,6-dimethyl-4-(trifluoromethyl)phenyl]-5-(trifluoromethyl)pyrimidin-4-ol, 4.861 g, 13.590 mmol) was added as a solid, portion-wise; the solution first became orange, then, at the end of the addition it became deep orange. The reaction mixture was heated at 60° C. overnight (20 h). Once cooled to room temperature, the reaction mixture was diluted with saturated sodium bicarbonate solution (120 mL) and stirred vigorously for 15 minutes. 25% Sodium hydroxide solution (13 mL) was added to reach pH ˜8-9. Layers were separated and aqueous layer was extracted with dichloromethane (2×150 mL). Organic layers were combined, dried over sodium sulfate and concentrated under reduced pressure to afford a mixture of N′-[4-chloro-6-[2,6-dimethyl-3-(trifluoromethyl)phenyl]-5-(trifluoromethyl)pyrimidin-2-yl]-N,N-dimethyl-formamidine and N′-[4-chloro-6-[2,6-dimethyl-4-(trifluoromethyl)phenyl]-5-(trifluoromethyl)pyrimidin-2-yl]-N,N-dimethyl-formamidine (5.69 g, 93%) as brown oil ¹H NMR and LCMS data are provided only for the major product in the mixture, N′-[4-chloro-6-[2,6-dimethyl-3-(trifluoromethyl)phenyl]-5-(trifluoromethyl)pyrimidin-2-yl]-N,N-dimethyl-formamidine, ¹H NMR (300 MHz, DMSO-d₆) δ 8.69 (s, 1H), 7.65 (d, J=8.2 Hz, 1H), 7.34 (d, J=7.9 Hz, 1H), 3.19 (s, 3H), 3.08 (s, 3H), 2.08-1.98 (m, 6H). ¹⁹F NMR (282 MHz, DMSO-d₆) δ −55.69 (s, 3F), −59.38 (s, 3F), ESI-MS m/z calc. 424.08893, found 425.1 (M+1)⁺; Retention time: 2.17 minutes; LC method K.

### Step 4: 4-chloro-6-[2,6-dimethyl-3-(trifluoromethyl)phenyl]-5-(trifluoromethyl)pyrimidin-2-amine

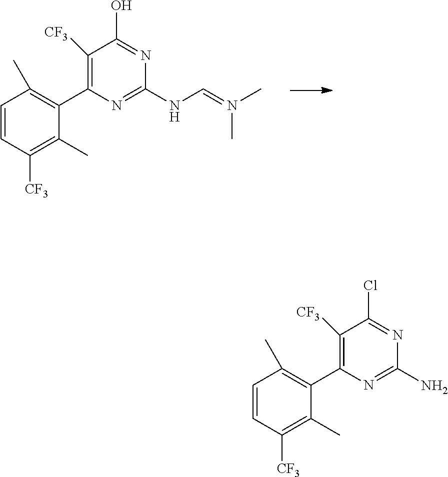

Concentrated hydrochloric acid (5.4 mL of 12 M, 64.800 mmol) was added to a solution of N′-[4-chloro-6-[2,6-dimethyl-3-(trifluoromethyl)phenyl]-5-(trifluoromethyl)pyrimidin-2-yl]-N,N-dimethyl-formamidine (6:1 mixture with N′-[4-chloro-6-[2,6-dimethyl-4-(trifluoromethyl)phenyl]-5-(trifluoromethyl)pyrimidin-2-yl]-N,N-dimethyl-formamidine, 4.32 g, 10.170 mmol) in isopropanol (40 mL) and the mixture was stirred at room temperature for 4 hours. After cooling to room temperature, the mixture was carefully transferred to a 0.5 L separatory funnel with ethyl acetate (150 mL), then water (80 mL) was added. The mixture was extracted with ethyl acetate (1×150 mL, then 2×100 mL). The combined organic layers were washed with water (100 mL) and brine (60 mL) then dried over sodium sulfate, filtered, and the solvent was removed under reduced pressure. The residue was purified by flash chromatography (dry-loaded, 80 g silica gel) eluting (0-12 CV) with mixtures of 0-15% ethyl acetate in heptanes. 4-Chloro-6-[2,6-dimethyl-3-(trifluoromethyl)phenyl]-5-(trifluoromethyl)pyrimidin-2-amine was isolated as a yellow foam in two separate batches (1.28 g, 30% and 1.06 g, 26%), the first batch being a 5:1 mixture with isomer 4-chloro-6-[2,6-dimethyl-4-(trifluoromethyl)phenyl]-5-(trifluoromethyl)pyrimidin-2-amine and the second batch being slightly more enriched in the major isomer. First batch: ESI-MS m/z calc. 369.04675, found 370.1 (M+1)⁺; Retention time: 2.19 minutes; LC method K, ¹H NMR (300 MHz, DMSO-d₆) δ 8.22-8.08 (m, 2H), 7.65 (d, J=7.9 Hz, 1H), 7.35 (d, J=8.2 Hz, 1H), 2.12-2.05 (m, 6H); and second batch ESI-MS m/z calc. 369.04675, found 370.1 (M+1)⁺; Retention time: 2.2 minutes; LC method K, ¹H NMR (300 MHz, DMSO-d₆) δ8.24-8.06 (m, 2H), 7.65 (d, J=8.2 Hz, 1H), 7.35 (d, J=8.2 Hz, 1H), 2.16-2.01 (m, 6H).

### Step 5: methyl 3-[[4-chloro-6-[2,6-dimethyl-3-(trifluoromethyl)phenyl]-5-(trifluoromethyl)pyrimidin-2-yl]sulfamoyl]benzoate

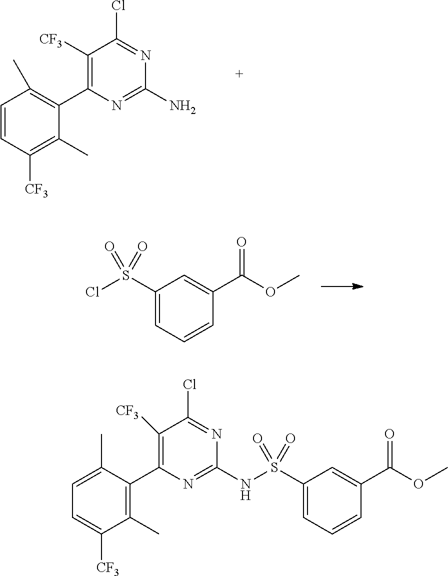

4-chloro-6-[2,6-dimethyl-3-(trifluoromethyl)phenyl]-5-(trifluoromethyl)pyrimidin-2-amine (1173 mg, 2.9362 mmol) was dissolved in MeTHF (13 mL) and cooled in an ice bath under stirring and nitrogen. To the cold solution methyl 3-chlorosulfonylbenzoate (970 mg, 3.9683 mmol) was added in one portion and, to the cold pale-yellow solution, a solution of lithium tert-butoxide (2.5 mL of 3 M, 7.5000 mmol) (in heptane) was added dropwise. The ice bath was removed, and the solution was stirred for 4 hours at room temperature. The reaction was quenched with HCl aqueous solution (1.5M, about 2.5-3.5 mL, up to pH 6-7) and the phases were separated. The aqueous phase was extracted with MeTHF (2×40 mL) and the organic phases were combined and washed with brine (20 mL) and dried over sodium sulfate. The solution was filtered and concentrated. The crude product (1.3 g, brown foam) was purified by flash chromatography (dry-loaded) (80 g silica gel) eluting with mixtures of 0%-20% ethyl acetate in heptanes over 14 CV and then 20%-100% over 5 CV and 100% ethyl acetate over 5 CV to give the following fractions:—fractions 80-98, methyl 3-[[4-chloro-6-[2,6-dimethyl-3-(trifluoromethyl)phenyl]-5-(trifluoromethyl)pyrimidin-2-yl]sulfamoyl]benzoate (200 mg, 10%) as off-white solid; ESI-MS m/z calc. 567.0454, found 568.1 (M+1)⁺; Retention time: 2.28 minutes; LC method K. ¹H NMR (300 MHz, CDCl₃) δ 8.69 (s, 1H), 8.29 (d, J=7.9 Hz, 1H), 8.20 (d, J=7.9 Hz, 1H), 7.63 (d, J=8.2 Hz, 1H), 7.52 (t, J=7.9 Hz, 1H), 7.20 (d, J=7.9 Hz, 1H), 3.92 (s, 3H), 1.94-1.80 (m, 6H).

### Step 6: 3-[[4-chloro-6-[2,6-dimethyl-3-(trifluoromethyl)phenyl]-5-(trifluoromethyl)pyrimidin-2-yl]sulfamoyl]benzoic acid

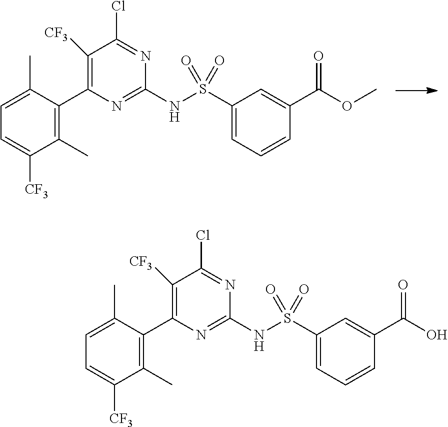 [SMILES: COC(=O)c1cccc(S(=O)(=O)Nc2nc(Cl)c(C(F)(F)F)c(-c3c(C)ccc(C(F)(F)F)c3C)n2)c1 ; Cc1ccc(C(F)(F)F)c(C)c1-c1nc(NS(=O)(=O)c2cccc(C(=O)O)c2)nc(Cl)c1C(F)(F)F]

A mixture of methyl 3-[[4-chloro-6-[2,6-dimethyl-3-(trifluoromethyl)phenyl]-5-(trifluoromethyl)pyrimidin-2-yl]sulfamoyl]benzoate (738 mg, 1.2905 mmol) in tetrahydrofuran (20 mL) and water (20 mL) was treated with lithium hydroxide hydrate (180 mg, 4.2036 mmol) and stirred at room temperature for 2-3 hours. Most of the tetrahydrofuran was removed under reduced pressure, and the remaining aqueous layer was acidified to a pH of about 4 using solid citric acid (155 mg). Transferred to a 120-mL separatory funnel with water (10 mL) and the aqueous layer was extracted with ethyl acetate (3×30 mL). The combined organic layers were washed with water (2×15 mL), brine (15 mL), dried over sodium sulfate, filtered and concentrated under reduced pressure. Gave 3-[[4-chloro-6-[2,6-dimethyl-3-(trifluoromethyl)phenyl]-5-(trifluoromethyl)pyrimidin-2-yl]sulfamoyl]benzoic acid (717 mg, 94%) as beige foaming solid. ESI-MS m/z calc. 553.0298, found 554.1 (M+1)⁺; Retention time: 2.13 minutes; LC method K. ¹H NMR (300 MHz, CDCl₃) δ 8.75 (s, 1H), 8.33 (d, J=7.9 Hz, 1H), 8.25 (d, J=7.9 Hz, 1H), 7.67-7.50 (m, 2H), 7.21 (d, J=7.9 Hz, 1H), 1.95-1.89 (m, 6H).

### Step 7: 3-[[4-[(2R)-2-(tert-butoxycarbonylamino)-4-methyl-pentoxy]-6-[2,6-dimethyl-3-(trifluoromethyl)phenyl]-5-(trifluoromethyl)pyrimidin-2-yl]sulfamoyl]benzoic acid

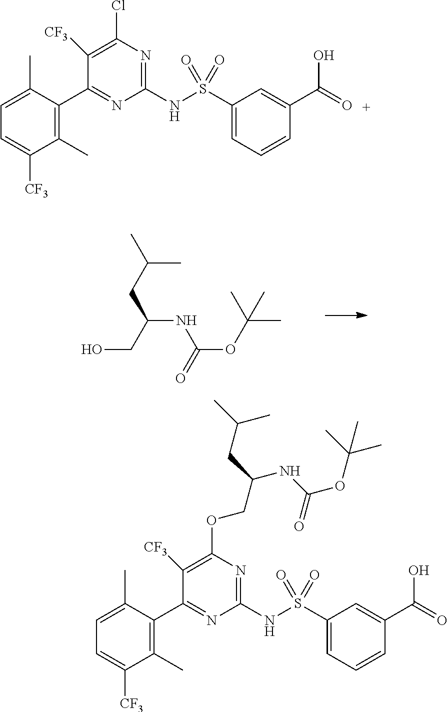

3-[[4-Chloro-6-[2,6-dimethyl-3-(trifluoromethyl)phenyl]-5-(trifluoromethyl)pyrimidin-2-yl]sulfamoyl]benzoic acid (150 mg, 0.2314 mmol) (also called as Boc-D-Leucinol) and tert-butyl N-[(1R)-1-(hydroxymethyl)-3-methyl-butyl]carbamate (60 mg, 0.2678 mmol) were combined and dissolved in tetrahydrofuran (2 mL). Sodium tert-butoxide (67 mg, 0.6763 mmol) was added. The reaction mixture was allowed to stir at 30° C. for 2 hours. The reaction mixture was diluted with EtOAc (80 mL) and washed with aqueous HCl (1 M, 1×0.7 mL) and water (10 mL) to reach pH=3-4, then with water (10 mL) and brine (10 mL). The organic layer was dried over anhydrous sodium sulfate, filtered and concentrated under reduced pressure. The crude product was purified by flash chromatography (dry-loaded) (24 g silica gel) eluting with mixtures of 0%-20% methanol in dichloromethane. Product eluted at 9% MeOH/DCM to give 3-[[4-[(2R)-2-(tert-butoxycarbonylamino)-4-methyl-pentoxy]-6-[2,6-dimethyl-3-(trifluoromethyl)phenyl]-5-(trifluoromethyl)pyrimidin-2-yl]sulfamoyl]benzoic acid (58 mg, 32%) as off-white foaming solid. ESI-MS m/z calc. 734.2209, found 635.2 (M−99)⁺; Retention time: 2.34 minutes; LC method K. ¹H NMR (300 MHz, CDCl₃) δ 9.01-8.72 (m, 1H), 8.38-8.23 (m, 1H), 8.21-8.07 (m, 1H), 7.58 (d, J=7.3 Hz, 2H), 7.21-7.08 (m, 1H), 4.83-4.42 (m, 2H), 4.25-4.10 (m, 1H), 3.69-3.58 (m, 1H), 3.20-3.05 (m, 2H), 2.09-1.93 (m, 6H), 1.68-1.58 (m, 1H), 1.37-1.13 (m, 9H), 1.00-0.87 (m, 6H).

### Step 8: (11R)-6-[2,6-Dimethyl-3-(trifluoromethyl)phenyl]-11-isobutyl-2,2-dioxo-7-(trifluoromethyl)-9-oxa-2λ⁶-thia-3,5,12,19-tetrazatricyclo[12.3.1.14,8]nonadeca-1(18),4(19),5,7,14,16-hexaen-13-one (Compound 163)

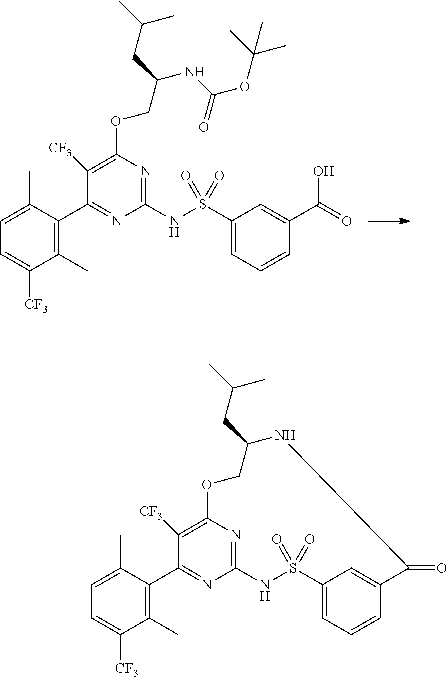 [SMILES: Cc1ccc(C(F)(F)F)c(C)c1-c1nc(NS(=O)(=O)c2cccc(C(=O)O)c2)nc(OC[C@@H](CC(C)C)NC(=O)OC(C)(C)C)c1C(F)(F)F ; Cc1ccc(C(F)(F)F)c(C)c1-c1nc2nc(c1C(F)(F)F)OC[C@@H](CC(C)C)NC(=O)c1cccc(c1)S(=O)(=O)N2]

3-[[4-[(2R)-2-(tert-Butoxycarbonylamino)-4-methyl-pentoxy]-6-[2,6-dimethyl-3-(trifluoromethyl)phenyl]-5-(trifluoromethyl)pyrimidin-2-yl]sulfamoyl]benzoic acid (57 mg, 0.0736 mmol) was dissolved in dioxane (1.8 mL). A solution of hydrochloric acid (0.2 mL of 4 M, 0.8000 mmol) in dioxane was added dropwise. The mixture was stirred at room temperature for 30 minutes. LCMS indicated little conversion rate. To the mixture was added again hydrochloric acid (0.4 mL of 4 M, 1.6000 mmol). More hydrochloric acid (0.2 mL of 4 M, 0.8000 mmol) was added and the reaction continue to stir at room temperature. The mixture was let to stir overnight at room temperature. Volatiles were removed under reduced pressure. The remaining residue was dissolved in dimethylformamide (2.5 mL). HATU (31 mg, 0.0815 mmol) was added followed by triethylamine (36.300 mg, 0.05 mL, 0.3587 mmol). After stirring at room temperature for 35 minutes, the solution was diluted with EtOAc (60 mL) and washed with aqueous HCl (1 M, 2×10 mL) and brine (3×20 mL). The organic layer was dried over sodium sulfate, filtered and concentrated under reduced pressure. The resulted off-white residue was purified using the following conditions: Gemini-NX 5u C₁₈ 110A 21,2×50 mm, Flow: 23.75 mL/min, Run time: 15 min, Mobile phase Condition: Initial 100% H₂O (10 mM ammonium formate+0.1% NH₄OH) and 0% CH₃CN for 3 min. Then, three linear gradients were applied. The first was an increase to 30% CH₃CN (0.1% formic Acid) for 1 min. The second increased to 70% CH₃CN for 7 min and the last increased to 95% CH₃CN for 2 min, then hold at 95% CH₃CN for 2 min. (11R)-6-[2,6-dimethyl-3-(trifluoromethyl)phenyl]-11-isobutyl-2,2-dioxo-7-(trifluoromethyl)-9-oxa-2λ⁶-thia-3,5,12,19-tetrazatricyclo[12.3.1.14,8]nonadeca-1(18),4(19),5,7,14,16-hexaen-13-one (18.2 mg, 38%) was isolated as a white solid. ESI-MS m/z calc. 616.1579, found 617.2 (M+1)⁺; Retention time: 3.41 minutes; LC method U. ¹H NMR (300 MHz, DMSO-d₆) δ 12.82 (br. s, 1H), 8.56 (s, 1H), 8.01 (d, J=6.8 Hz, 1H), 7.97 (d, J=10.0 Hz, 1H), 7.82-7.73 (m, 2H), 7.67 (d, J=7.9 Hz, 1H), 7.51 (d, J=17.0 Hz, 1H), 7.35 (dd, J=17.8, 7.8 Hz, 1H), 5.36-5.24 (m, 1H), 4.10-3.94 (m, 1H), 2.19-2.04 (m, 3H), 1.95-1.80 (m, 3H), 1.64-1.40 (m, 2H), 1.27-1.12 (m, 1H), 0.83-0.69 (m, 3H), 0.31-0.18 (m, 3H).

### Example 58: Preparation of Compound 164

### Step 1: 3-[[4-[(2R)-2-Amino-4-methyl-pentoxy]-6-chloro-5-(trifluoromethyl)pyrimidin-2-yl]sulfamoyl]benzoic acid

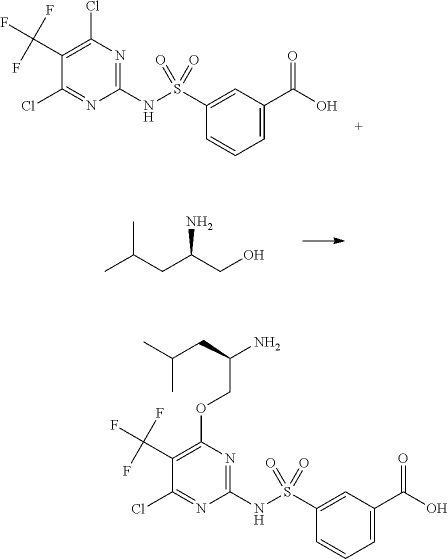

3-[[4,6-Dichloro-5-(trifluoromethyl)pyrimidin-2-yl]sulfamoyl]benzoic acid (160 mg, 0.3845 mmol) and (2R)-2-amino-4-methyl-pentan-1-ol (45 mg, 0.3840 mmol) were combined in THE (2 mL) and cooled in a dry ice:acetone bath. sodium tert-butoxide (152 mg, 1.582 mmol) was added and the reaction was stirred for 2 h. The reaction was diluted with 1M HCl and extracted with ethyl acetate. The organics were washed with brine, dried over sodium sulfate and evaporated. The crude material was purified by reverse-phase chromatography utilizing a gradient of 10-99% acetonitrile in 5 mM aq HCl to yield 3-[[4-[(2R)-2-amino-4-methyl-pentoxy]-6-chloro-5-(trifluoromethyl)pyrimidin-2-yl]sulfamoyl]benzoic acid (hydrochloride salt)(92 mg, 45%) ESI-MS m/z calc. 496.0795, found 497.1 (M+1)⁺; Retention time: 0.48 minutes. ESI-MS m/z calc. 496.0795, found 497.1 (M+1)⁺; Retention time: 0.48 minutes, LC method D.

### Step 2: (11R)-6-Chloro-11-isobutyl-2,2-dioxo-7-(trifluoromethyl)-9-oxa-2λ⁶-thia-3,5,12,19-tetrazatricyclo[12.3.1.14,8]nonadeca-1(18),4,6,8(19),14,16-hexaen-13-one

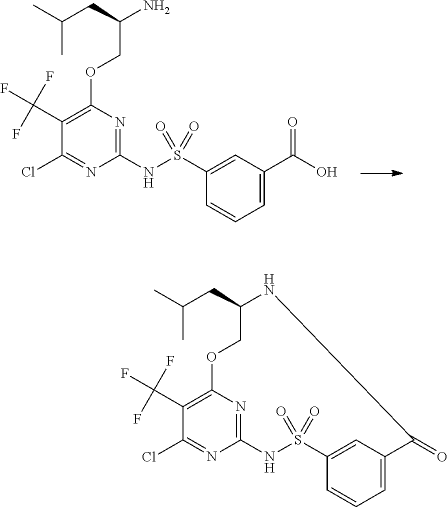 [SMILES: CC(C)C[C@@H](N)COc1nc(NS(=O)(=O)c2cccc(C(=O)O)c2)nc(Cl)c1C(F)(F)F ; CC(C)C[C@@H]1COc2nc(nc(Cl)c2C(F)(F)F)NS(=O)(=O)c2cccc(c2)C(=O)N1]

3-[[4-[(2R)-2-Amino-4-methyl-pentoxy]-6-chloro-5-(trifluoromethyl)pyrimidin-2-yl]sulfamoyl]benzoic acid (hydrochloride salt)(92 mg, 0.1725 mmol) and [dimethylamino(triazolo[4,5-b]pyridin-3-yloxy)methylene]-dimethyl-ammonium;hexafluorophosphate (72 mg, 0.1894 mmol) were combined in DMF (2 mL) and triethylamine (75 μL, 0.5381 mmol) was added. The reaction was stirred at room temperature for 1 h. The reaction was filtered and purified by reverse phase HPLC utilizing a gradient of 1-99% acetonitrile in 5 mM aq HCl to yield a mixture of the product and HOAt added to the product. The reaction was moved forward as is. (11R)-6-Chloro-11-isobutyl-2,2-dioxo-7-(trifluoromethyl)-9-oxa-2λ⁶-thia-3,5,12,19-tetrazatricyclo[12.3.1.14,8]nonadeca-1(18),4,6,8(19),14,16-hexaen-13-one (33.3 mg, 40%) ESI-MS m/z calc. 478.06894, found 479.0 (M+1)⁺; Retention time: 0.62 minutes. ESI-MS m/z calc. 478.06894, found 479.0 (M+1)⁺; Retention time: 0.62 minutes, LC method D.

### Step 3: (11R)-11-Isobutyl-6-(2-isopropylphenyl)-2,2-dioxo-7-(trifluoromethyl)-9-oxa-2λ⁶-thia-3,5,12,19-tetrazatricyclo[12.3.1.14,8]nonadeca-1(18),4,6,8(19),14,16-hexaen-13-one (Compound 164)

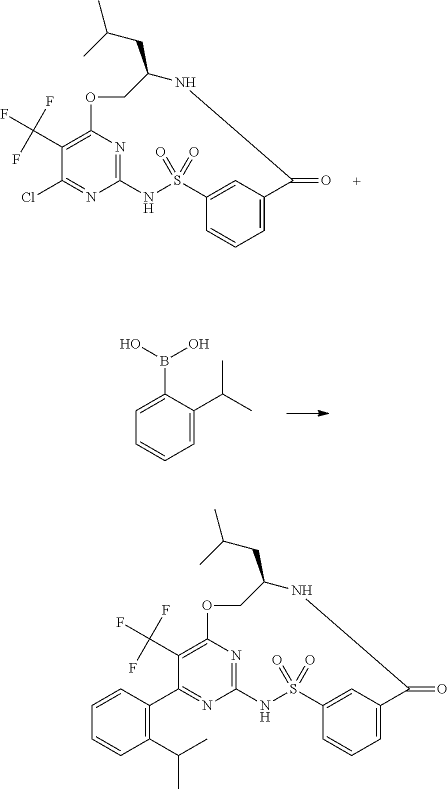 [SMILES: CC(C)C[C@@H]1COc2nc(nc(Cl)c2C(F)(F)F)NS(=O)(=O)c2cccc(c2)C(=O)N1 ; CC(C)c1ccccc1B(O)O ; CC(C)C[C@@H]1COc2nc(nc(-c3ccccc3C(C)C)c2C(F)(F)F)NS(=O)(=O)c2cccc(c2)C(=O)N1]

(11R)-6-Chloro-11-isobutyl-2,2-dioxo-7-(trifluoromethyl)-9-oxa-2λ⁶-thia-3,5,12,19-tetrazatricyclo[12.3.1.14,8]nonadeca-1(18),4,6,8(19),14,16-hexaen-13-one (8.5 mg, 0.01775 mmol), (2-isopropylphenyl)boronic acid (8.2 mg, 0.05000 mmol), tetrakis(triphenylphosphane)palladium(0) (4.1 mg, 0.003548 mmol), and 2 M sodium carbonate (36 μL of 2 M, 0.07200 mmol) were combined in DME (0.9 mL) and heated at 120° C. for 2 h in a screw top vial. The reaction mixture was cooled, filtered, and purified by reverse phase HPLC utilizing a gradient of 1-99% acetonitrile in 5 mM aqueous HCl to yield (11R)-11-isobutyl-6-(2-isopropylphenyl)-2,2-dioxo-7-(trifluoromethyl)-9-oxa-2λ⁶-thia-3,5,12,19-tetrazatricyclo[12.3.1.14,8]nonadeca-1(18),4,6,8(19),14,16-hexaen-13-one (2.8 mg, 27%). ESI-MS m/z calc. 562.18616, found 563.3 (M+1)⁺; Retention time: 1.8 minutes; LC method A.

### Example 59: Preparation of Compound 165

### Step 1: tert-Butyl N-tert-butoxycarbonyl-N-(4,6-dichloro-5-isopropoxy-pyrimidin-2-yl)carbamate

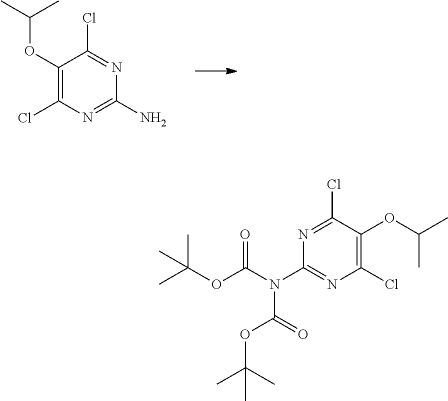 [SMILES: CC(C)Oc1c(Cl)nc(N)nc1Cl ; CC(C)Oc1c(Cl)nc(N(C(=O)OC(C)(C)C)C(=O)OC(C)(C)C)nc1Cl]

4,6-Dichloro-5-isopropoxy-pyrimidin-2-amine (980 mg, 4.413 mmol) and Boc anhydride (2.061 g, 9.443 mmol) were combined in DCM (7.5 mL), followed by DMAP (46.2 mg, 0.3782 mmol). The reaction mixture was stirred at room temperature for 16 h. The reaction mixture was diluted with DCM (25 mL) and washed with water, then brine. The organics were dried over sodium sulfate and evaporated to give tert-butyl N-tert-butoxycarbonyl-N-(4,6-dichloro-5-isopropoxy-pyrimidin-2-yl)carbamate (1.663 g, 89%). ESI-MS m/z calc. 421.11713, found 422.1 (M+1)⁺; Retention time: 0.85 minutes; LC method D. ¹H NMR (400 MHz, Chloroform-d) δ 4.69 (hept, J=6.1 Hz, 1H), 1.47 (s, 18H), 1.41 (d, J=6.2 Hz, 6H).

### Step 2: tert-Butyl N-tert-butoxycarbonyl-N-[4-chloro-6-(2,6-dimethylphenyl)-5-isopropoxy-pyrimidin-2-yl]carbamate

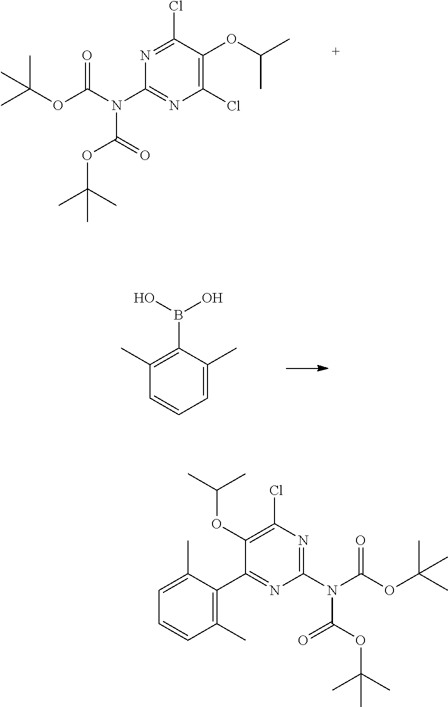

tert-Butyl N-tert-butoxycarbonyl-N-(4,6-dichloro-5-isopropoxy-pyrimidin-2-yl)carbamate (966 mg, 2.287 mmol), (2,6-dimethylphenyl)boronic acid (380.8 mg, 2.539 mmol), cesium carbonate (1.981 g, 6.080 mmol), and dichloro[1,1′-bis(diphenylphosphino)ferrocene]palladium(II)dichloromethane adduct (202.3 mg, 0.2477 mmol) were combined in water (1 mL) and DME (6 mL) and heated at 95° C. for 16 h. The reaction mixture was partitioned between ethyl acetate and water. The organics were separated, washed with brine, dried over sodium sulfate and evaporated. The crude material was purified by silica gel chromatography eluting with 0-30% ethyl acetate in hexanes to give tert-butyl N-tert-butoxycarbonyl-N-[4-chloro-6-(2,6-dimethylphenyl)-5-isopropoxy-pyrimidin-2-yl]carbamate (517 mg, 46%). ESI-MS m/z calc. 491.2187, found 492.4 (M+1)⁺; Retention time: 0.91 minutes; LC method D.

### Step 3: 4-Chloro-6-(2,6-dimethylphenyl)-5-isopropoxy-pyrimidin-2-amine

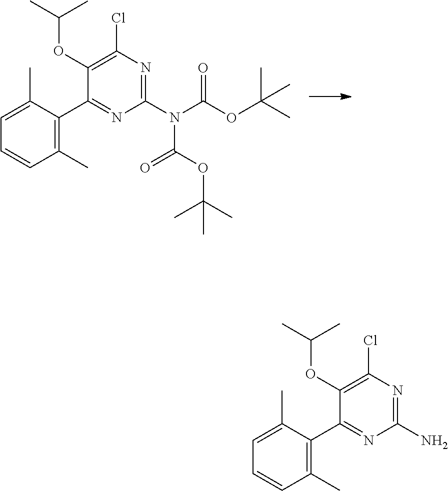 [SMILES: Cc1cccc(C)c1-c1nc(N(C(=O)OC(C)(C)C)C(=O)OC(C)(C)C)nc(Cl)c1OC(C)C ; Cc1cccc(C)c1-c1nc(N)nc(Cl)c1OC(C)C]

tert-Butyl N-tert-butoxycarbonyl-N-[4-chloro-6-(2,6-dimethylphenyl)-5-isopropoxy-pyrimidin-2-yl]carbamate (428 mg, 0.8699 mmol) and HCl in dioxane (2 mL of 4 M, 8.000 mmol) were combined in dichloromethane (5 mL) and stirred for 16 h. The reaction mixture was evaporated to dryness to give 4-chloro-6-(2,6-dimethylphenyl)-5-isopropoxy-pyrimidin-2-amine (hydrochloride salt) (281.7 mg, 99%). ESI-MS m/z calc. 291.11383, found 292.1 (M+1)⁺; Retention time: 0.68 minutes; LC method D.

### Step 4: 3-[[4-Chloro-6-(2,6-dimethylphenyl)-5-isopropoxy-pyrimidin-2-yl]sulfamoyl]benzoic acid

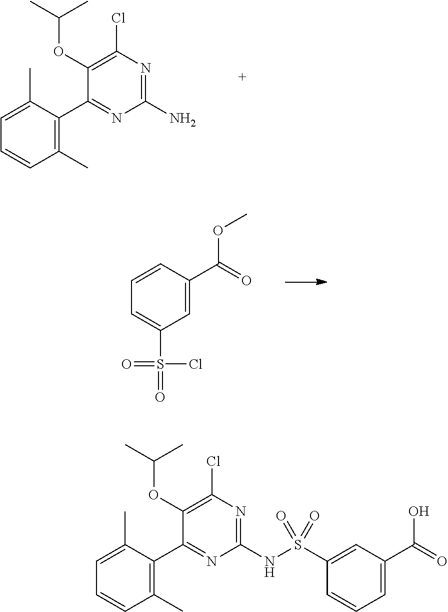 [SMILES: Cc1cccc(C)c1-c1nc(N)nc(Cl)c1OC(C)C ; COC(=O)c1cccc(S(=O)(=O)Cl)c1 ; Cc1cccc(C)c1-c1nc(NS(=O)(=O)c2cccc(C(=O)O)c2)nc(Cl)c1OC(C)C]

4-Chloro-6-(2,6-dimethylphenyl)-5-isopropoxy-pyrimidin-2-amine (257 mg, 0.8808 mmol) was dissolved in THF (3 mL) and cooled in an ice bath. methyl 3-chlorosulfonylbenzoate (657 mg, 2.800 mmol) was added in one portion. lithium tert-amoxide (1.6 mL of 40% w/w, 4.966 mmol) was added dropwise and the reaction was allowed to slowly warm to room temperature. The reaction was stirred for 5 h, then made acidic by the addition of 1M HCl. The reaction mixture was extracted with ethyl acetate. The organics were washed with brine, dried over sodium sulfate and evaporated. The crude material was purified by silica gel chromatography eluting with 0-100% ethyl acetate in hexanes to give methyl 3-[[4-chloro-6-(2,6-dimethylphenyl)-5-isopropoxy-pyrimidin-2-yl]sulfamoyl]benzoate (278 mg). Product from above was dissolved in THF (3 mL) and NaOH (3 mL of 1 M, 3.000 mmol) and stirred at room temperature for 2 h. The reaction mixture was made acidic by the addition of 1 M HCl and extracted with ethyl acetate. The organics were separated, washed with brine, dried over sodium sulfate and evaporated to give 3-[[4-chloro-6-(2,6-dimethylphenyl)-5-isopropoxy-pyrimidin-2-yl]sulfamoyl]benzoic acid (203 mg, 48%) (yield for 2 steps). ESI-MS m/z calc. 475.09686, found 476.1 (M+1)⁺; Retention time: 0.68 minutes; LC method D.

### Step 5: (11R)-6-(2,6-Dimethylphenyl)-11-isobutyl-7-isopropoxy-2,2-dioxo-9-oxa-2λ⁶-thia-3,5,12,19-tetrazatricyclo[12.3.1.14,8]nonadeca-1(18),4(19),5,7,14,16-hexaen-13-one (Compound 165)

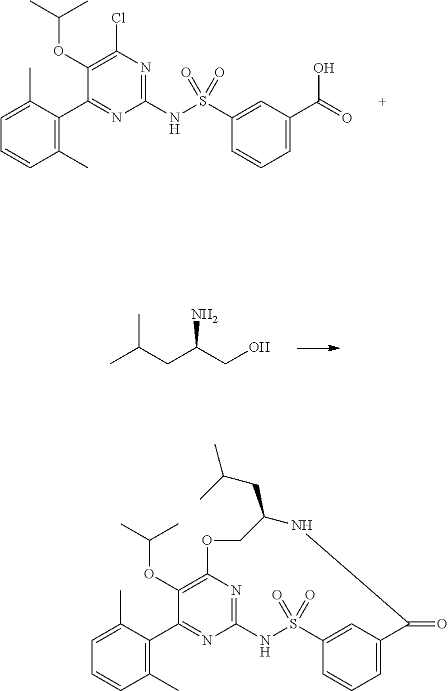

3-[[4-Chloro-6-(2,6-dimethylphenyl)-5-isopropoxy-pyrimidin-2-yl]sulfamoyl]benzoic acid (44.8 mg, 0.09413 mmol) and (2R)-2-amino-4-methyl-pentan-1-ol (15.3 mg, 0.1306 mmol) were combined in THF (1 mL). Sodium tert-butoxide (52.6 mg, 0.5473 mmol) was added and the reaction was heated at 45° C. for 1 h. The reaction was filtered (a lot of material was lost at this point) and purified by reverse phase HPLC utilizing a gradient of 1-99% acetonitrile in 5 mM aqueous HCl to yield 3-[[4-[(2R)-2-amino-4-methyl-pentoxy]-6-(2,6-dimethylphenyl)-5-isopropoxy-pyrimidin-2-yl]sulfamoyl]benzoic acid (hydrochloride salt) (7.3 mg, 13%).

3-[[4-[(2R)-2-amino-4-methyl-pentoxy]-6-(2,6-dimethylphenyl)-5-isopropoxy-pyrimidin-2-yl]sulfamoyl]benzoic acid (hydrochloride salt) (7.3 mg, 0.012 mmol) and HATU (9.1 mg, 0.02393 mmol) were combined in DMF (1 mL) and triethylamine (26 μL, 0.1865 mmol) was added. The reaction was stirred for 30 min, filtered and purified by reverse phase HPLC utilizing a gradient of 1-99% acetonitrile in 5 mM aqueous HCl to yield (11R)-6-(2,6-dimethylphenyl)-11-isobutyl-7-isopropoxy-2,2-dioxo-9-oxa-2λ⁶-thia-3,5,12,19-tetrazatricyclo[12.3.1.14,8]nonadeca-1(18),4(19),5,7,14,16-hexaen-13-one (4 mg, 8%). Yield over 2 steps. ESI-MS m/z calc. 538.225, found 539.3 (M+1)⁺; Retention time: 1.71 minutes; LC method A.

### Example 60: Preparation of Compound 166

### Step 1: tert-Butyl N-tert-butoxycarbonyl-N-(4,6-dichloro-5-methoxy-pyrimidin-2-yl)carbamate

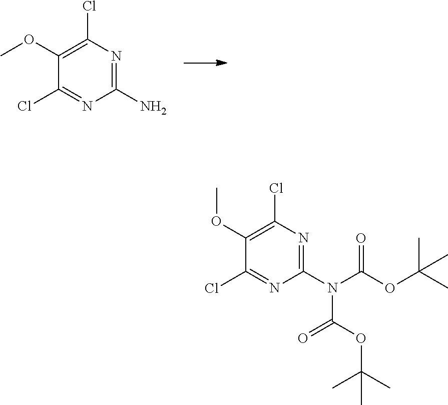 [SMILES: COc1c(Cl)nc(N)nc1Cl ; COc1c(Cl)nc(N(C(=O)OC(C)(C)C)C(=O)OC(C)(C)C)nc1Cl]

To a solution of 4,6-dichloro-5-methoxy-pyrimidin-2-amine (11.73 g, 60.458 mmol) in DCM (200 mL) was added DMAP (591 mg, 4.8376 mmol) and Boc₂O (27.7 g, 29.158 mL, 126.92 mmol). The reaction was stirred for 3 hours at room temperature before being washed with brine (200 mL) and water (200 mL). The organic layer was concentrated in vacuum and dried over sodium sulfate to give tert-butyl N-tert-butoxycarbonyl-N-(4,6-dichloro-5-methoxy-pyrimidin-2-yl)carbamate (21.55 g, 90%). ESI-MS m/z calc. 393.08582, found 394.0 (M+1)⁺; Retention time: 3.44 minutes; LC method T.

### Step 2: tert-Butyl N-tert-butoxycarbonyl-N-[4-chloro-6-(2,6-dimethylphenyl)-5-methoxy-pyrimidin-2-yl]carbamate

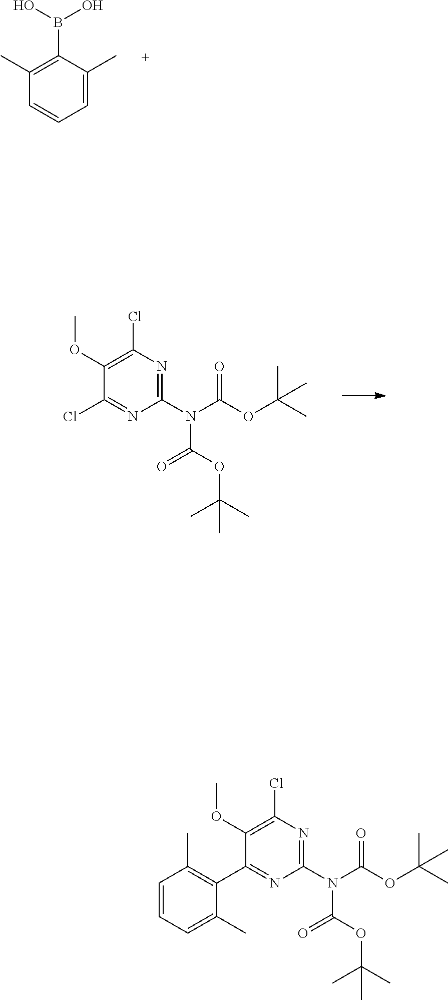

To a solution of tert-butyl N-tert-butoxycarbonyl-N-(4,6-dichloro-5-methoxy-pyrimidin-2-yl)carbamate (31.58 g, 80.101 mmol) dissolved in DME (225 mL) and water (31 mL) was added (2,6-dimethylphenyl)boronic acid (16.5 g, 110.01 mmol) and cesium carbonate (68 g, 208.71 mmol) at room temperature. The solution was stirred for 10 min while being bubbled with a nitrogen stream. Then Pd(dppf)Cl₂ (5.86 g, 8.0087 mmol) was added to the solution and heated to 80° C. overnight. The solution was cooled to room temperature before being diluted with water (250 mL) and extracted with ethyl acetate (2×300 mL). The combined organic layer was washed with brine (400 mL) and dried over sodium sulfate before being concentrated under vacuum. The organic residue was purified by silica gel chromatography eluting 0-60% ethyl acetate-hexanes to give tert-butyl N-tert-butoxycarbonyl-N-[4-chloro-6-(2,6-dimethylphenyl)-5-methoxy-pyrimidin-2-yl]carbamate (50.35 g, 135%). ESI-MS m/z calc. 463.1874, found 464.2 (M+1)⁺; Retention time: 3.68 minutes; LC method T.

### Step 3: 4-Chloro-6-(2,6-dimethylphenyl)-5-methoxy-pyrimidin-2-amine

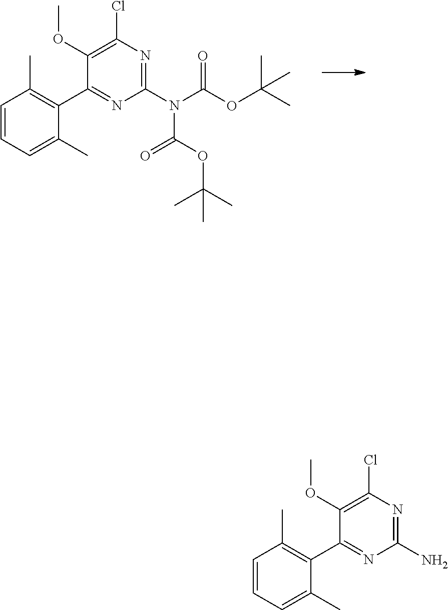 [SMILES: COc1c(Cl)nc(N(C(=O)OC(C)(C)C)C(=O)OC(C)(C)C)nc1-c1c(C)cccc1C ; COc1c(Cl)nc(N)nc1-c1c(C)cccc1C]

To a solution of tert-butyl N-tert-butoxycarbonyl-N-[4-chloro-6-(2,6-dimethylphenyl)-5-methoxy-pyrimidin-2-yl]carbamate (50.35 g, 108.52 mmol) in DCM (500 mL) was added HCl (100 mL of 4 M, 400.00 mmol) in dioxane. The solution was stirred at room temperature overnight before being concentrated in vacuum. The residue was then basified with sodium bicarbonate (400 mL) and extracted with ethyl acetate (500 mL). The organic layer was washed with brine (500 mL) and dried over sodium sulfate. The organic phase was concentrated then triturated with hexanes (2×50 mL) to give 4-chloro-6-(2,6-dimethylphenyl)-5-methoxy-pyrimidin-2-amine (10.16 g, 36%). ESI-MS m/z calc. 263.08255, found 264.1 (M+1)⁺; Retention time: 2.73 minutes; LC method T.

### Step 4: Methyl 3-[[4-chloro-6-(2,6-dimethylphenyl)-5-methoxy-pyrimidin-2-yl]sulfamoyl]benzoate

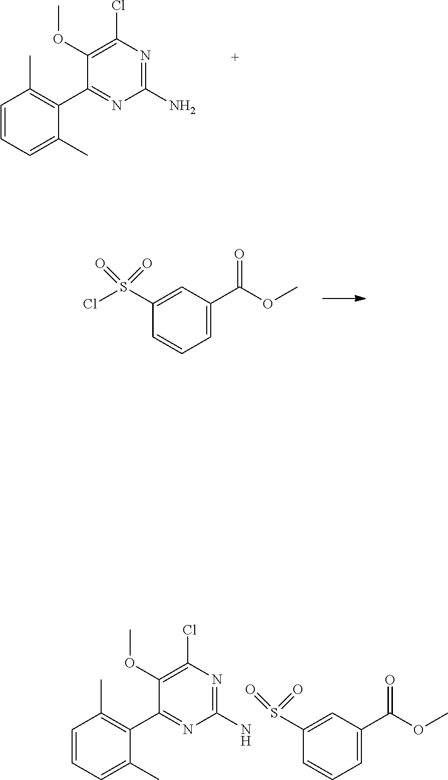

To a solution of 4-chloro-6-(2,6-dimethylphenyl)-5-methoxy-pyrimidin-2-amine (223 mg, 0.8456 mmol) in THF (6 mL) at 0° C. was added methyl 3-chlorosulfonylbenzoate (496 mg, 2.1137 mmol). Then Lithium tert-amoxide (584.00 mg, 2 mL of 40% w/w, 2.4830 mmol) was added to the solution dropwise keeping the temperature below 5° C. The solution was allowed to warm to room temperature while it stirred for 3 hours. The solution was acidified with 1M HCl (5 mL) and extracted with ethyl acetate (20 mL). The organic layer was washed with brine (20 mL) and dried over sodium sulfate. The organic layer was then concentrated in vacuum to give methyl 3-[[4-chloro-6-(2,6-dimethylphenyl)-5-methoxy-pyrimidin-2-yl]sulfamoyl]benzoate (386 mg, 99%) of a yellow solid. ESI-MS m/z calc. 461.0812, found 462.1 (M+1)⁺; Retention time: 3.18 minutes; LC method T.

### Step 5: 3-[[4-Chloro-6-(2,6-dimethylphenyl)-5-methoxy-pyrimidin-2-yl]sulfamoyl]benzoic acid

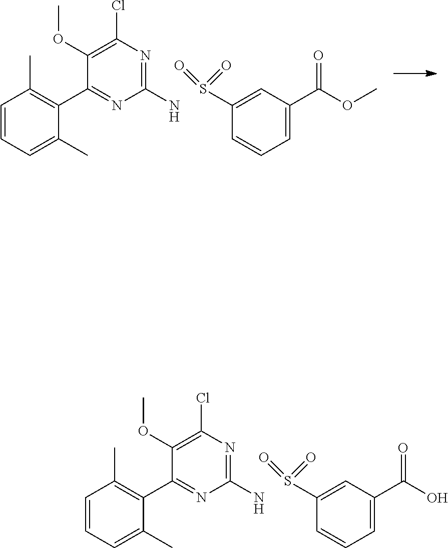

To a solution of methyl 3-[[4-chloro-6-(2,6-dimethylphenyl)-5-methoxy-pyrimidin-2-yl]sulfamoyl]benzoate (386 mg, 0.8356 mmol) in THF (10 mL) was added an aqueous solution of NaOH (5 mL of 1 M, 5.0000 mmol) and stirred for 1 hour at room temperature. The solution was acidified using 1M HCl (5 mL) and extracted with ethyl acetate (2×20 mL) before being washed with brine (20 mL). The organic layer was dried over sodium sulfate and concentrated in vacuum to give 3-[[4-chloro-6-(2,6-dimethylphenyl)-5-methoxy-pyrimidin-2-yl]sulfamoyl]benzoic acid (314 mg, 84%) as a white solid. ESI-MS m/z calc. 447.06558, found 448.1 (M+1)⁺; Retention time: 2.9 minutes; LC method T.

### Step 6: 3-[[4-[(2R)-2-Amino-4-methyl-pentoxy]-6-(2,6-dimethylphenyl)-5-methoxy-pyrimidin-2-yl]sulfamoyl]benzoic acid

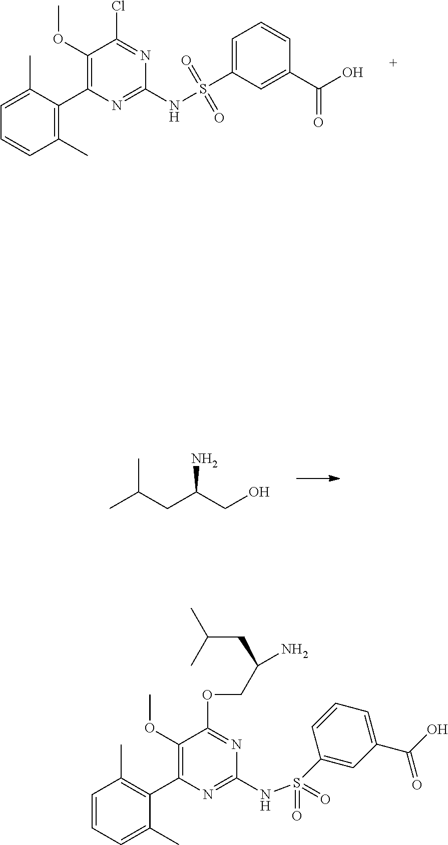

In a 25 mL flask, to a stirred solution of 3-[[4-chloro-6-(2,6-dimethylphenyl)-5-methoxy-pyrimidin-2-yl]sulfamoyl]benzoic acid (500 mg, 1.116 mmol) in anhydrous tetrahydofuran (12 mL) was added a solution of (2R)-2-amino-4-methyl-pentan-1-ol (225 mg, 1.920 mmol) in anhydrous tetrahydrofuran (0.5 mL). The heterogeneous mixture was stirred for 2 min while purging nitrogen through it, to form a uniform milky emulsion. To the emulsion, was added sodium tert-butoxide (625 mg, 6.503 mmol) at once. The reaction was stirred for 40 min at room temperature. The reaction mixture was partitioned between ethyl acetate (30 mL) and a ice-cold hydrochloric acid (9 mL of 1 M, 9.000 mmol) (pH was about 2). The aqueous layer was re-extracted with ethyl acetate (2×20 mL). The combined organics were washed with brine (10 mL), dried over anhydrous sodium sulfate, filtered and concentrated under reduced pressure to obtain crude material. It was taken up in DMSO 4 mL), micro-filtered, and purified by reverse-phase HPLC (C₁₈ column, 1-99% acetonitrile in water over 15 min, HCl as modifier, two injections). The desired fractions were combined to obtain 3-[[4-[(2R)-2-amino-4-methyl-pentoxy]-6-(2,6-dimethylphenyl)-5-methoxy-pyrimidin-2-yl]sulfamoyl]benzoic acid (hydrochloride salt) (369 mg, 59%) as white solid. ESI-MS m/z calc. 528.2043, found 529.0 (M+1)⁺; Retention time: 1.13 minutes; LC method A.

### Step 7: tert-Butyl 2-[(11R)-6-(2,6-dimethylphenyl)-11-isobutyl-7-methoxy-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate

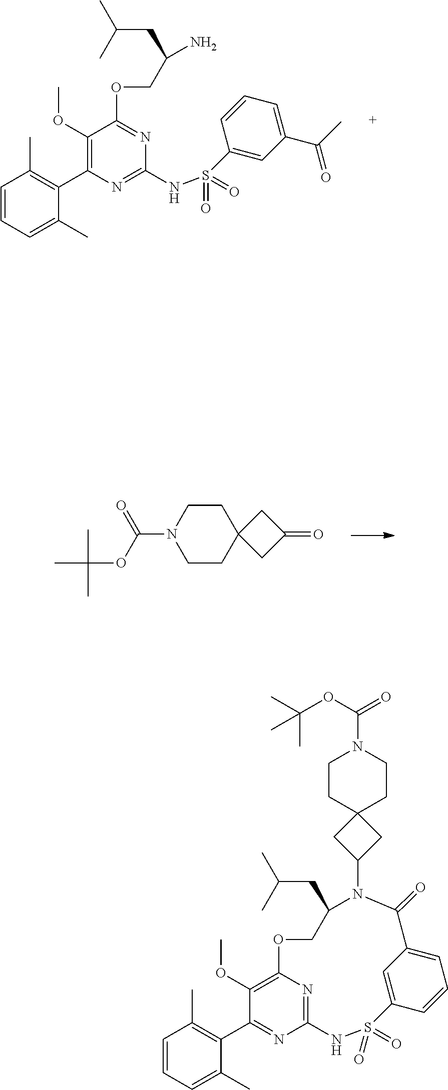

In a 4 mL vial to a stirred mixture of 3-[[4-[(2R)-2-amino-4-methyl-pentoxy]-6-(2,6-dimethylphenyl)-5-methoxy-pyrimidin-2-yl]sulfamoyl]benzoic acid (hydrochloride salt) (100 mg, 0.1770 mmol) and tert-butyl 2-oxo-7-azaspiro[3.5]nonane-7-carboxylate (45 mg, 0.1880 mmol) in anhydrous dichloromethane (0.5 mL) was added sodium triacetoxyborohydride (155 mg, 0.7313 mmol). The vial was briefly purged with nitrogen and the mixture was stirred at ambient temperature for 20 h (overnight). Then methanol (0.2 mL) and water (0.2 mL) were added in that order, and the mixture was concentrated under reduced pressure. The residue was taken up in DMSO (2 mL), micro-filtered, and purified by reverse-phase HPLC (1-99% acetonitrile in water over 15 min, HCl as modifier) to give 3-[[4-[(2R)-2-[(7-tert-butoxycarbonyl-7-azaspiro[3.5]nonan-2-yl)amino]-4-methyl-pentoxy]-6-(2,6-dimethylphenyl)-5-methoxy-pyrimidin-2-yl]sulfamoyl]benzoic acid (hydrochloride salt) (70 mg, 50%) as white solid. ESI-MS m/z calc. 751.3615, found 752.2 (M+1)⁺; Retention time: 0.56 minutes; LC method D.

In a 20 mL vial, to a stirred solution of the above acid in anhydrous DMF (3 mL) were added [dimethylamino(triazolo[4,5-b]pyridin-3-yloxy)methylene]-dimethyl-ammonium (Phosphorus Hexafluoride Ion) (83 mg, 0.2183 mmol) (HATU) and DIEA (150 μL, 0.8612 mmol), in that order. Nitrogen gas was purged for 20 sec and capped. The solution was stirred at ambient temperature for 30 min. The contents were diluted with DMSO (0.5 mL), micro-filtered, and purified from prep reverse-phase HPLC (C₁₈ column, 1-99% acetonitrile in water over 15 min, HCl as modifier.) to furnish tert-butyl 2-[(11R)-6-(2,6-dimethylphenyl)-11-isobutyl-7-methoxy-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (28 mg, 22%) as white solid. ESI-MS m/z calc. 733.3509, found 734.2 (M+1)⁺; Retention time: 2.24 minutes (LC method A).

### Step 8: (11R)-12-(7-Azaspiro[3.5]nonan-2-yl)-6-(2,6-dimethylphenyl)-11-isobutyl-7-methoxy-2,2-dioxo-9-oxa-2λ⁶-thia-3,5,12,19-tetrazatricyclo[12.3.1.14,8]nonadeca-1(18),4(19),5,7,14,16-hexaen-13-one

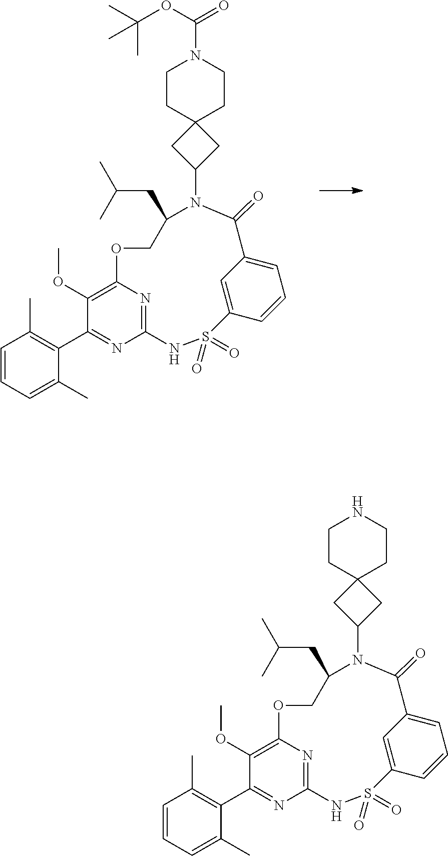 [SMILES: COc1c2nc(nc1-c1c(C)cccc1C)NS(=O)(=O)c1cccc(c1)C(=O)N(C1CC3(CCN(C(=O)OC(C)(C)C)CC3)C1)[C@H](CC(C)C)CO2 ; COc1c2nc(nc1-c1c(C)cccc1C)NS(=O)(=O)c1cccc(c1)C(=O)N(C1CC3(CCNCC3)C1)[C@H](CC(C)C)CO2]

To a stirred solution of tert-butyl 2-[(11R)-6-(2,6-dimethylphenyl)-11-isobutyl-7-methoxy-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (28 mg, 0.03815 mmol) in anhydrous dichloromethane (1 mL) was added hydrogen chloride in dioxane (300 μL of 4.0 M, 1.200 mmol) under nitrogen at ambient temperature. The pale-yellow solution was stirred for 30 min, then concentrated under reduced pressure and dried further in vacuo to give (11R)-12-(7-azaspiro[3.5]nonan-2-yl)-6-(2,6-dimethylphenyl)-11-isobutyl-7-methoxy-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (13 mg, 51%) as off-white solid. ESI-MS m/z calc. 633.29846, found 634.5 (M+1)⁺; Retention time: 1.29 minutes; LC method A.

### Step 9: (11R)-6-(2,6-Dimethylphenyl)-11-isobutyl-7-methoxy-12-[7-(2-methoxyethyl)-7-azaspiro[3.5]nonan-2-yl]-2,2-dioxo-9-oxa-2×⁶-thia-3,5,12,19-tetrazatricyclo[12.3.1.14,8]nonadeca-1(18),4(19),5,7,14,16-hexaen-13-one (Compound 166)

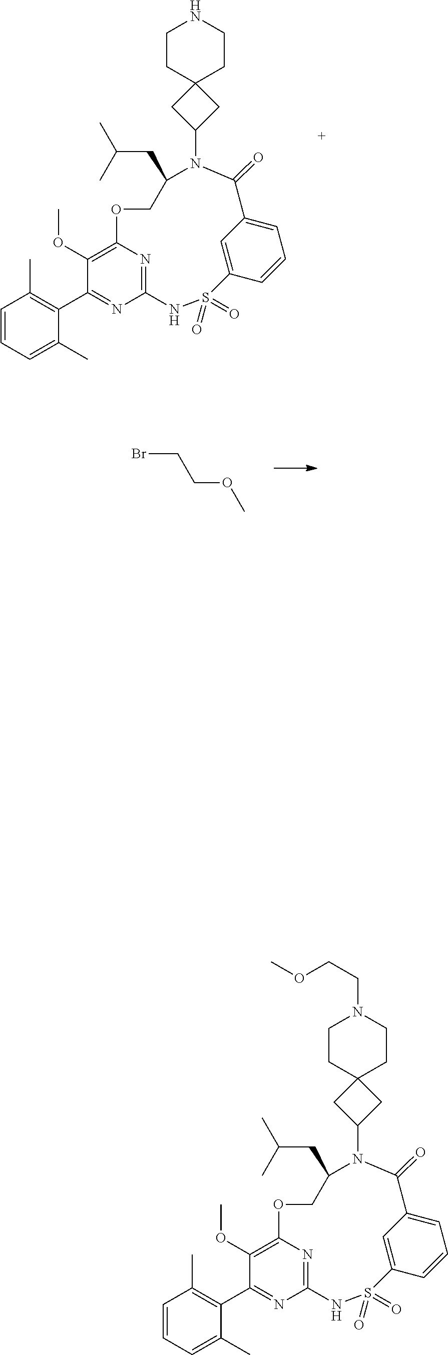

In a 4 mL screw-cap vial, to a stirred solution of (11R)-12-(7-azaspiro[3.5]nonan-2-yl)-6-(2,6-dimethylphenyl)-11-isobutyl-7-methoxy-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (13 mg, 0.01940 mmol) in anhydride acetonitrile (0.2 mL) were added a solution of 1-bromo-2-methoxy-ethane (7 mg, 0.05036 mmol) in anhydrous acetonitrile (0.1 mL), followed by addition of N,N-diisopropyl ethyl amine (20 μL, 0.1148 mmol). The vial was capped under nitrogen and the heterogeneous mixture was allowed to stir at 55° C. The suspension gradually went into the solution within 20 min and the reaction solution was stirred overnight (16 h). The reaction was allowed to cool to ambient temperature and concentrated under reduced pressure. The residue was taken up in dimethylsulfoxide (1 mL), micro-filtered, and purified by preparative reverse-phase HPLC(C₁₈, 1-99% acetonitrile in water over 15 min, HCl as modifier) to furnish (11R)-6-(2,6-dimethylphenyl)-11-isobutyl-7-methoxy-12-[7-(2-methoxyethyl)-7-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (4.3 mg, 30%) as white solid. ESI-MS m/z calc. 691.34033, found 692.3 (M+1)⁺; Retention time: 1.38 minutes; LC method A.

### Example 61: Preparation of Compound 167

### Step 1: 3-[[4-Chloro-6-(2,6-dimethylphenyl)-5-fluoro-pyrimidin-2-yl]sulfamoyl]benzoic acid

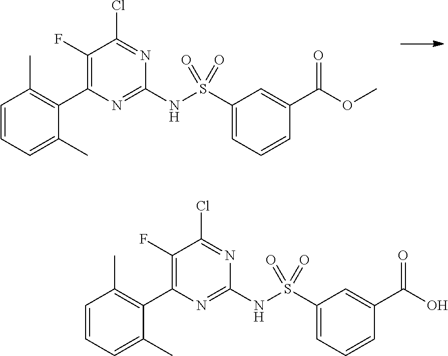 [SMILES: COC(=O)c1cccc(S(=O)(=O)Nc2nc(Cl)c(F)c(-c3c(C)cccc3C)n2)c1 ; Cc1cccc(C)c1-c1nc(NS(=O)(=O)c2cccc(C(=O)O)c2)nc(Cl)c1F]

To a stirring solution of methyl 3-[[4-chloro-6-(2,6-dimethylphenyl)-5-fluoro-pyrimidin-2-yl]sulfamoyl]benzoate (490 mg, 1.0892 mmol) in THF (9 mL) at room temperature was added an aqueous solution of NaOH (4.5 mL of 1 M, 4.5000 mmol). The reaction mixture was stirred at this temperature for 2 hours. Water was added (20 mL), and the reaction mixture was acidified with 2 M aqueous HCl to pH ˜1. The product was extracted with ethyl acetate (3×40 mL). The combined organic layers were washed with brine (30 mL), dried over anhydrous sodium sulfate and concentrated to afford 3-[[4-chloro-6-(2,6-dimethylphenyl)-5-fluoro-pyrimidin-2-yl]sulfamoyl]benzoic acid (463 mg, 93%) as white solid. ESI-MS m/z calc. 435.0456, found 436.4 (M+1)⁺; Retention time: 5.21 minutes; LC method S.

### Step 2: (2R)-1-(4-tert-Butylphenyl)-2-(dibenzylamino)propan-1-one

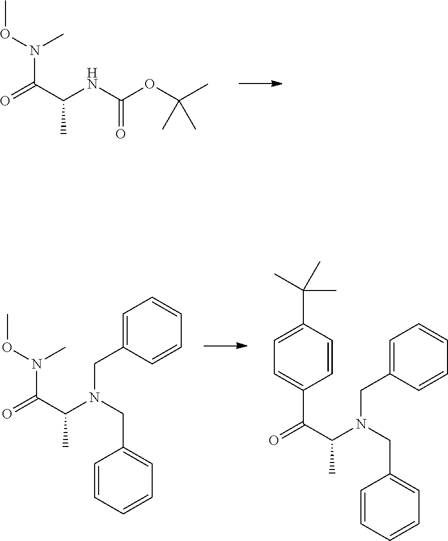 [SMILES: CON(C)C(=O)[C@@H](C)NC(=O)OC(C)(C)C ; CON(C)C(=O)[C@@H](C)N(Cc1ccccc1)Cc1ccccc1 ; C[C@H](C(=O)c1ccc(C(C)(C)C)cc1)N(Cc1ccccc1)Cc1ccccc1]

In a 500-mL round-bottomed flask, tert-butyl N-[(1R)-2-[methoxy(methyl)amino]-1-methyl-2-oxo-ethyl]carbamate (15.05 g, 64.79 mmol) was mixed with dioxane (80 mL), to which a dioxane solution of HCl (80 mL of 4.0 M, 320.0 mmol) was added. This mixture was stirred at room temperature for 16 h, then at 70° C. for 30 min. It was then evaporated to dryness in vacuo to give a brown gum. In a 500-mL round-bottomed flask, the crude product was dissolved in DMF (200 mL) and treated with potassium carbonate (60.70 g, 439.2 mmol) and BnBr (20 mL, 168.2 mmol). This mixture was stirred at room temperature for 21 h, after which it was quenched by slowly pouring onto cold 1 N HCl solution (450 mL) to achieve a pH of ˜11 (CAUTION: GAS EVOLUTION). The resulting mixture was extracted with ethyl acetate (3×300 mL). The combined organic extracts were washed with water (400 mL) and saturated aqueous sodium chloride solution (300 mL), then dried over sodium sulfate, filtered, and evaporated in vacuo. This liquid was mixed with THF (50 mL), and then evaporated in vacuo while stirring to give an orange liquid: (2R)-2-(dibenzylamino)-N-methoxy-N-methyl-propanamide (22.346 g, 110%) ESI-MS m/z calc. 312.18378, found 313.3 (M+1)⁺; Retention time: 0.99 minutes (LC method A).

In a 1-L round-bottomed flask, the crude product was dissolved in THF (100 mL) and placed in a room-temperature water bath. A THF solution of bromo-(4-tert-butylphenyl)magnesium (200 mL of 0.5 M, 100.0 mmol) was added in two portions, and the resulting mixture was stirred at room temperature for 2 h. It was then quenched by pouring onto cold 1 N HCl (300 mL) and extracted with ethyl acetate (3×300 mL). The combined organic extracts were washed with water (300 mL) and saturated aqueous sodium chloride solution (300 mL), then dried over sodium sulfate, filtered, and evaporated in vacuo. The resulting brown liquid (˜35 mL) was purified by silica gel chromatography (330 g of silica) using a gradient eluent of 0 to 20% ethyl acetate in hexanes to give a yellow liquid that solidifies under high vacuum: (2R)-1-(4-tert-butylphenyl)-2-(dibenzylamino)propan-1-one (14.5744 g, 58%). ESI-MS m/z calc. 385.24057, found 386.4 (M+1)⁺; Retention time: 1.84 minutes; LC method A. ¹H NMR (400 MHz, dimethylsulfoxide-d6) δ 7.57-7.50 (m, 2H), 7.45-7.39 (m, 2H), 7.31-7.22 (m, 6H), 7.18-7.11 (m, 4H), 4.28 (q, J=6.6 Hz, 1H), 3.54 (AB quartet, ΔδAB=0.019, JAB=13.8 Hz, 4H), 1.32 (s, 9H), 1.25 (d, J=6.7 Hz, 3H)

### Step 3: (1R,2R)-2-Amino-1-(4-tert-butylphenyl)propan-1-ol

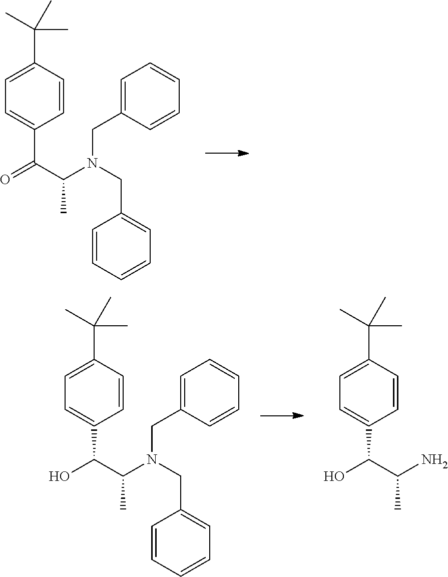

In a 500-mL round-bottomed flask, (2R)-1-(4-tert-butylphenyl)-2-(dibenzylamino)propan-1-one (14.5744 g, 37.80 mmol) was dissolved in THF (50 mL), and MeOH (100 mL) was added. This solution was cooled to −20° C. (dry ice/brine bath), after which sodium borohydride (2.5 g, 66.08 mmol) was added in one portion. The resulting mixture was stirred at −20° C. for 90 min. It was then quenched with 1 N HCl (75 mL), neutralized with saturated aqueous sodium bicarbonate solution (150 mL), and extracted with ethyl acetate (3×250 mL). The combined organic extracts was washed with water (400 mL) and saturated aqueous sodium chloride solution (300 mL), then dried over sodium sulfate, filtered, and evaporated in vacuo to give a white solid: (1R,2R)-1-(4-tert-butylphenyl)-2-(dibenzylamino)propan-1-ol (14.99 g, 91%). ESI-MS m/z calc. 387.25623, found 388.4 (M+1)⁺; Retention time: 1.51 minutes; LC method A. ¹H NMR (400 MHz, dimethylsulfoxide-d6) δ 7.35-7.31 (m, 8H), 7.29-7.26 (m, 2H), 7.26-7.22 (m, 2H), 7.08-7.03 (m, 2H), 4.88 (d, J=1.5 Hz, 1H), 4.47 (d, J=8.6 Hz, 1H), 3.88 (d, J=13.7 Hz, 2H), 3.45 (d, J=13.6 Hz, 2H), 2.67 (dq, J=8.6, 6.6 Hz, 1H), 1.25 (s, 9H), 0.81 (d, J=6.7 Hz, 3H)

In a 250-mL round-bottomed flask equipped with a magnetic stir bar, the product was dissolved in THF (50 mL), and EtOH (50 mL) was added. The air above the solution was replaced with nitrogen by three vacuum/nitrogen sequences. The cap was briefly removed, and Pd(OH)₂/C (5.0 g of 10% w/w, 3.560 mmol) was added. This reaction mixture was stirred under hydrogen (3 L, 119.1 mmol) at 50° C. for 20 h and at 65° C. for 5.5 h. After this time, the reaction mixture was cooled to room temperature, filtered through Celite and rinsed with methanol (400 mL). This solution was evaporated in vacuo, then dissolved in THF (150 mL). This solution was evaporated to dryness in vacuo while stirring to give a yellow solid, (1R,2R)-2-amino-1-(4-tert-butylphenyl)propan-1-ol (7.895 g, 68%). ESI-MS m/z calc. 207.16231, found 208.2 (M+1)⁺; Retention time: 0.91 minutes; LC method A. ¹H NMR (400 MHz, dimethylsulfoxide-d6) δ 7.33 (d, J=8.4 Hz, 2H), 7.21 (d, J=8.3 Hz, 2H), 4.07 (d, J=7.0 Hz, 1H), 2.79 (p, J=6.6 Hz, 1H), 1.27 (s, 9H), 0.77 (d, J=6.5 Hz, 3H)

### Step 4: (10R,11R)-10-(4-tert-Butylphenyl)-6-(2,6-dimethylphenyl)-7-fluoro-11-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 167)

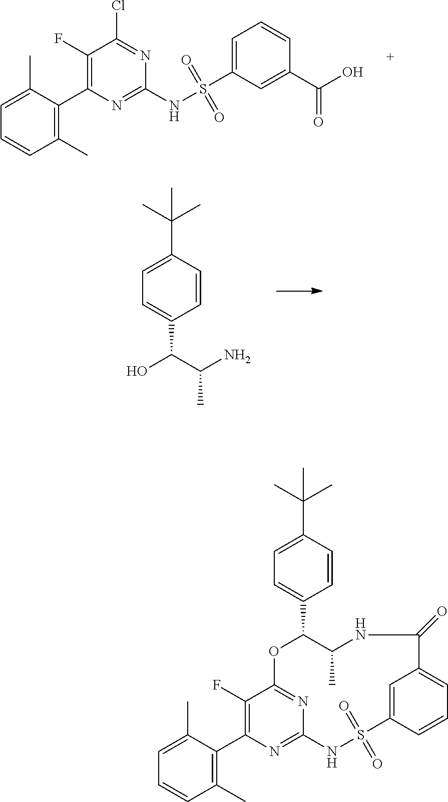 [SMILES: Cc1cccc(C)c1-c1nc(NS(=O)(=O)c2cccc(C(=O)O)c2)nc(Cl)c1F ; C[C@@H](N)[C@H](O)c1ccc(C(C)(C)C)cc1 ; Cc1cccc(C)c1-c1nc2nc(c1F)O[C@H](c1ccc(C(C)(C)C)cc1)[C@@H](C)NC(=O)c1cccc(c1)S(=O)(=O)N2]

3-[[4-Chloro-6-(2,6-dimethylphenyl)-5-fluoro-pyrimidin-2-yl]sulfamoyl]benzoic acid (52.7 mg, 0.1209 mmol), (1R,2R)-2-amino-1-(4-tert-butylphenyl)propan-1-ol (30.4 mg, 0.1437 mmol), and sodium tert-butoxide (65.2 mg, 0.6784 mmol) were combined in THF (2 mL) and stirred at room temperature for 1 h. The reaction was added dropwise to a stirred solution of HATU (93.2 mg, 0.2451 mmol) in DMF (2 mL) and the reaction was stirred an additional 2 h. The reaction was partitioned between ethyl acetate and a 1M HCl solution. The organics were separated, washed with brine, dried over sodium sulfate, and evaporated. The crude material was purified by reverse phase HPLC utilizing a gradient of 1-99% acetonitrile in 5 mM aqueous HCl to yield (10R,11R)-10-(4-tert-butylphenyl)-6-(2,6-dimethylphenyl)-7-fluoro-11-methyl-2,2-dioxo-9-oxa-2λ⁶-thia-3,5,12,19-tetrazatricyclo[12.3.1.14,8]nonadeca-1(18),4(19),5,7,14,16-hexaen-13-one (21.8 mg, 30%) ESI-MS m/z calc. 588.22064, found 589.4 (M+1)⁺; Retention time: 2.14 minutes (LC method A).

### Example 62: Preparation of Compound 168

### Step 1: 3-[[5-Bromo-4-(2,6-dimethylphenyl)-6-[(2R)-4-methyl-2-(spiro[2.3]hexan-5-ylamino)pentoxy]pyrimidin-2-yl]sulfamoyl]benzoic acid

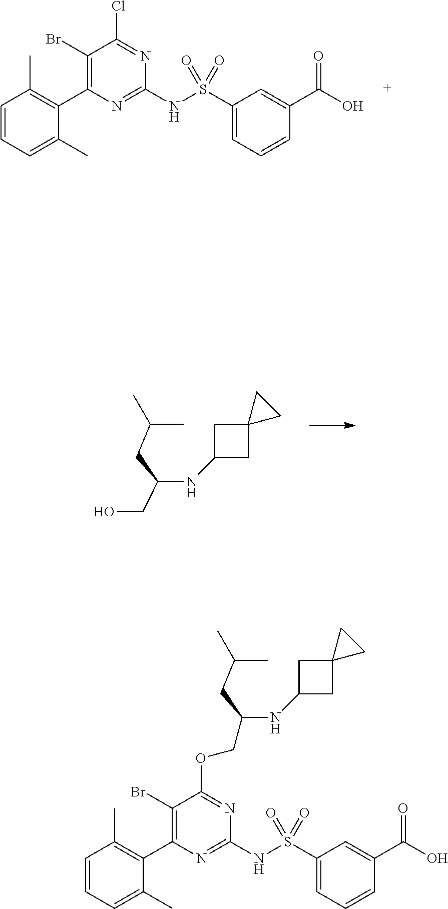 [SMILES: Cc1cccc(C)c1-c1nc(NS(=O)(=O)c2cccc(C(=O)O)c2)nc(Cl)c1Br ; CC(C)C[C@H](CO)NC1CC2(CC2)C1 ; Cc1cccc(C)c1-c1nc(NS(=O)(=O)c2cccc(C(=O)O)c2)nc(OC[C@@H](CC(C)C)NC2CC3(CC3)C2)c1Br]

To a stirring solution of 3-[[5-bromo-4-chloro-6-(2,6-dimethylphenyl)pyrimidin-2-yl]sulfamoyl]benzoic acid (1.310 g, 2.6371 mmol) and (2R)-4-methyl-2-(spiro[2.3]hexan-5-ylamino)pentan-1-ol (520 mg, 2.6354 mmol) in anhydrous THF (10 mL) at ambient temperature under argon was added sodium tert-butoxide (1.042 g, 10.842 mmol) in one portion. The reaction mixture was stirred at this temperature for 1.5 hours. The reaction mixture was quenched with aqueous HCl solution (1N, 15 mL) and stirred for 10 minutes. Hexanes (70 mL) were added and the reaction mixture was vigorously stirred for 1 hour. Solvent layer was decanted and the solid residue was dried under vacuum to afford 3-[[5-bromo-4-(2,6-dimethylphenyl)-6-[(2R)-4-methyl-2-(spiro[2.3]hexan-5-ylamino)pentoxy]pyrimidin-2-yl]sulfamoyl]benzoic acid (hydrochloride salt) (1.75 g, 75%) as a white solid. The product was used directly in the next step without further purification. ESI-MS m/z calc. 656.1668, found 657.5 (M+1)⁺; Retention time: 5.26 minutes; LC method S.

### Step 2: (11R)-7-Bromo-6-(2,6-dimethylphenyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one

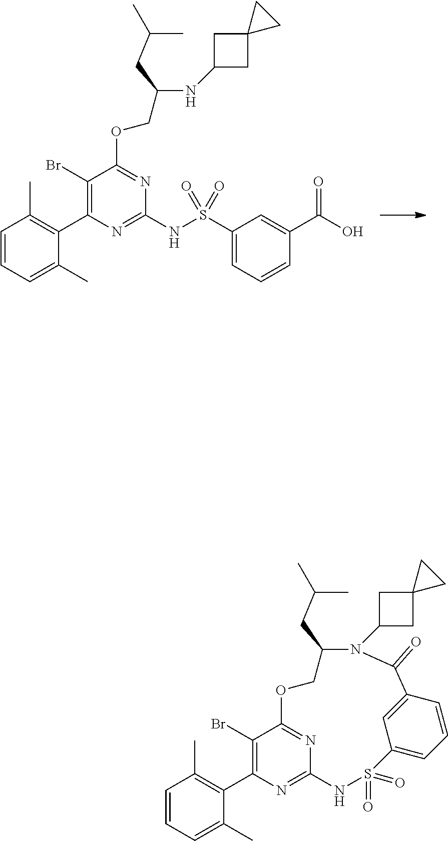 [SMILES: Cc1cccc(C)c1-c1nc(NS(=O)(=O)c2cccc(C(=O)O)c2)nc(OC[C@@H](CC(C)C)NC2CC3(CC3)C2)c1Br ; Cc1cccc(C)c1-c1nc2nc(c1Br)OC[C@@H](CC(C)C)N(C1CC3(CC3)C1)C(=O)c1cccc(c1)S(=O)(=O)N2]

To a stirring solution of crude product of 3-[[5-bromo-4-(2,6-dimethylphenyl)-6-[(2R)-4-methyl-2-(spiro[2.3]hexan-5-ylamino)pentoxy]pyrimidin-2-yl]sulfamoyl]benzoic acid (hydrochloride salt) (13.119 g, 18.902 mmol) in anhydrous NMP (180 mL) at room temperature under argon was added DIEA (10.017 g, 13.5 mL, 77.505 mmol) followed by HATU (9.4 g, 24.722 mmol). The reaction mixture was stirred at this temperature for 24 hours. The reaction mixture was poured into a mixture of aqueous HCl solution (1N, 150 mL) and water (150 mL), and the product was extracted with ethyl acetate (3×300 mL). The combined organic layers were washed with water (100 mL), brine (100 mL), dried over anhydrous sodium sulfate and concentrated. The crude product obtained was purified by flash chromatography (loaded in DCM) (330 g silica gel, eluting 0 to 30% ethyl acetate in hexane) to afford (11R)-7-bromo-6-(2,6-dimethylphenyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (6.84 g, 53%) as a white solid. ESI-MS m/z calc. 638.15625, found 639.3 (M+1)⁺; Retention time: 3.38 minutes; LC method W. ¹H NMR (500 MHz, DMSO-d₆) δ 12.41 (s, 1H), 8.46 (d, J=1.9 Hz, 1H), 7.99 (d, J=7.7 Hz, 1H), 7.82 (d, J=7.6 Hz, 1H), 7.76 (t, J=7.7 Hz, 1H), 7.24 (t, J=7.6 Hz, 1H), 7.15 (d, J=7.6 Hz, 1H), 7.09 (d, J=7.6 Hz, 1H), 5.18 (dd, J=10.7, 4.4 Hz, 1H), 4.53 (t, J=11.2 Hz, 1H), 4.27 (p, J=8.5 Hz, 1H), 3.64 (t, J=10.4 Hz, 1H), 3.26 (t, J=8.6 Hz, 2H), 2.11 (dt, J=22.1, 8.5 Hz, 2H), 2.03 (s, 3H), 1.76 (s, 3H), 1.74-1.67 (m, 1H), 1.29 (dtt, J=12.8, 9.0, 4.7 Hz, 1H), 1.24-1.14 (m, 1H), 0.71 (d, J=6.6 Hz, 3H), 0.51 (d, J=6.7 Hz, 2H), 0.47 (d, J=5.2 Hz, 2H), 0.17 (d, J=6.3 Hz, 3H).

### Step 3: Methyl (11R)-6-(2,6-dimethylphenyl)-11-isobutyl-2,2,13-trioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaene-7-carboxylate

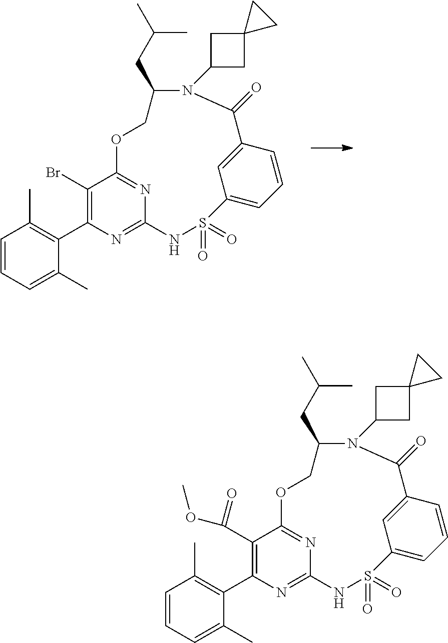 [SMILES: Cc1cccc(C)c1-c1nc2nc(c1Br)OC[C@@H](CC(C)C)N(C1CC3(CC3)C1)C(=O)c1cccc(c1)S(=O)(=O)N2 ; COC(=O)c1c2nc(nc1-c1c(C)cccc1C)NS(=O)(=O)c1cccc(c1)C(=O)N(C1CC3(CC3)C1)[C@H](CC(C)C)CO2]

To a solution of (11R)-7-bromo-6-(2,6-dimethylphenyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (3.73 g, 5.8317 mmol) in the mixture of MeOH (25 mL) and DMSO (15 mL) in the sealed vessel was added Pd(OAc)₂ (262 mg, 1.1670 mmol) dppf (647 mg, 1.1671 mmol) and TEA (2.3958 g, 3.3 mL, 23.676 mmol). The resulting solution was then pressurized to 250 psi with carbon monoxide gas. The mixture was stirred at 100° C. for 3 days. The reaction solution was cooled to ambient temperature and most of methanol was removed under reduced pressure. Water (100 mL) and ethyl acetate (100 mL) were added. The organic layer was separated, and aqueous layer was extracted with ethyl acetate (100 mL×2). The combined organic layers were washed with brine (30 mL×2), dried over sodium sulfate, filtered and concentrated under reduced pressure. The crude product was purified by reverse HPLC (60-100% mobile B; mobile A: 0.1% TFA in water; mobile B: 0.1% TFA in acetonitrile). Pure fractions were combined, and acetonitrile was removed under reduced pressure to afford methyl (11R)-6-(2,6-dimethylphenyl)-11-isobutyl-2,2,13-trioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaene-7-carboxylate (1.7 g, 45%) as a light brown solid. ESI-MS m/z calc. 618.2512, found 619.4 (M+1)⁺; Retention time: 3.0 minutes; LC method W. ¹H NMR (500 MHz, DMSO-d₆) δ 8.44 (s, 1H), 7.98 (d, J=7.7 Hz, 1H), 7.80 (d, J=7.6 Hz, 1H), 7.74 (t, J=7.7 Hz, 1H), 7.22 (t, J=7.6 Hz, 1H), 7.11 (d, J=7.6 Hz, 1H), 7.05 (d, J=7.6 Hz, 1H), 5.19 (dd, J=10.7, 4.3 Hz, 1H), 4.45 (t, J=11.2 Hz, 1H), 4.24 (p, J=8.5 Hz, 1H), 3.67 (t, J=9.5 Hz, 1H), 3.43 (s, 3H), 3.25 (p, J=8.6, 7.8 Hz, 2H), 2.16-2.09 (m, 2H), 2.08 (s, 3H), 1.77 (s, 3H), 1.66 (t, J=11.6 Hz, 1H), 1.34-1.22 (m, 2H), 0.74 (d, J=6.0 Hz, 3H), 0.54-0.48 (m, 2H), 0.46 (d, J=5.0 Hz, 2H), 0.19 (d, J=5.7 Hz, 3H).

### Step 4: (11R)-6-(2,6-Dimethylphenyl)-11-isobutyl-2,2,13-trioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaene-7-carboxylic acid (Compound 168)

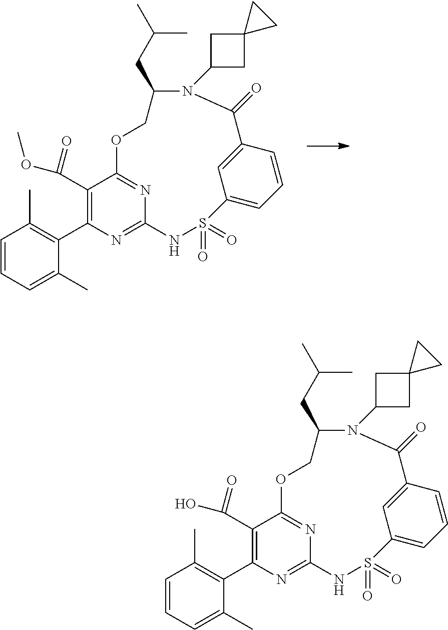 [SMILES: COC(=O)c1c2nc(nc1-c1c(C)cccc1C)NS(=O)(=O)c1cccc(c1)C(=O)N(C1CC3(CC3)C1)[C@H](CC(C)C)CO2 ; Cc1cccc(C)c1-c1nc2nc(c1C(=O)O)OC[C@@H](CC(C)C)N(C1CC3(CC3)C1)C(=O)c1cccc(c1)S(=O)(=O)N2]

To a solution of methyl (11R)-6-(2,6-dimethylphenyl)-11-isobutyl-2,2,13-trioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaene-7-carboxylate (46.7 mg, 0.0755 mmol) in the mixture of THF (0.4 mL) and MeOH (0.2 mL) was added NaOH (0.4 mL of 1 M, 0.4000 mmol). The resulting solution was heated at 50° C. for 19 hours, then at 80° C. for 24 hours. Reaction solution was cooled to ambient temperature, and water (10 mL) was added. The solution was washed with ether (10 mL×2). The aqueous layer was acidified with aqueous HCl (1N), then extracted with ethyl acetate (3×15 mL). The combined organic layers were washed with brine (5 mL×2), dried over anhydrous sodium sulfate, filtered and concentrated. The residue was purified by reverse HPLC (20% to 80% mobile B; mobile A: 0.1% TFA in water, 0.1% TFA in acetonitrile) to afford (11R)-6-(2,6-dimethylphenyl)-11-isobutyl-2,2,13-trioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaene-7-carboxylic acid (14.7 mg, 31%) as a white solid. ESI-MS m/z calc. 604.23553, found 605.6 (M+1)⁺; Retention time: 2.42 minutes; LC method W. ¹H NMR (500 MHz, DMSO-d₆) δ 13.12 (s, 1H), 8.42 (s, 1H), 7.95 (d, J=7.7 Hz, 1H), 7.73 (dd, J=18.4, 10.9 Hz, 2H), 7.20 (t, J=7.6 Hz, 1H), 7.09 (d, J=7.7 Hz, 1H), 7.03 (d, J=7.6 Hz, 1H), 6.53 (s, 1H), 5.16 (dd, J=10.7, 4.4 Hz, 1H), 4.45 (t, J=11.1 Hz, 1H), 4.24 (p, J=8.5 Hz, 1H), 3.75-3.59 (m, 1H), 3.25 (ddd, J=14.7, 12.1, 7.4 Hz, 2H), 2.10 (m, 5H), 1.80 (s, 3H), 1.64 (t, J=11.2 Hz, 1H), 1.26 (dt, J=10.8, 6.8 Hz, 2H), 0.71 (d, J=5.8 Hz, 3H), 0.57-0.38 (m, 4H), 0.17 (d, J=5.3 Hz, 3H).

### Example 63: Preparation of Compound 169

### Step 1: (11R)-6-(2,6-Dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaene-7-carbonitrile (Compound 169)

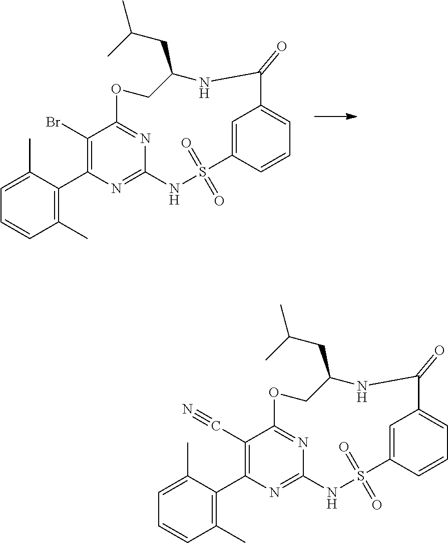 [SMILES: Cc1cccc(C)c1-c1nc2nc(c1Br)OC[C@@H](CC(C)C)NC(=O)c1cccc(c1)S(=O)(=O)N2 ; Cc1cccc(C)c1-c1nc2nc(c1C#N)OC[C@@H](CC(C)C)NC(=O)c1cccc(c1)S(=O)(=O)N2]

In a microwave reaction vial, (11R)-7-bromo-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (156.7 mg, 0.2801 mmol) was dissolved in dimethyl formamide (1.5 mL) along with zinc;dicyanide (65.8 mg, 0.5603 mmol) and the reaction was purged with nitrogen. To the reaction vial, Pd₂(dba)₃ (25.7 mg, 0.02807 mmol) and ferrous cyclopenta-1,4-dien-1-yl(diphenyl)phosphane (dppf, 31.1 mg, 0.05610 mmol) were added and the reaction was purged again with nitrogen. The reaction mixture was heated at 140° C. for 1 h. in the microwave. The reaction was evaporated to dryness then partitioned between ethyl acetate and water. The organic layer was isolated, dried over anhydrous sodium sulfate, filtered, and evaporated to dryness. The crude material was purified by preparative HPLC using 30-100% water/ACN gradient with HCl modifier. (11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaene-7-carbonitrile (13.5 mg, 9%). ESI-MS m/z calc. 505.17838, found 506.1 (M+1)⁺; Retention time: 1.58 minutes; (LC method A).

### Example 64: Preparation of Compound 170

### Step 1: (11R)-6-(2,6-Dimethylphenyl)-11-isobutyl-2,2,13-trioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaene-7-carbaldehyde

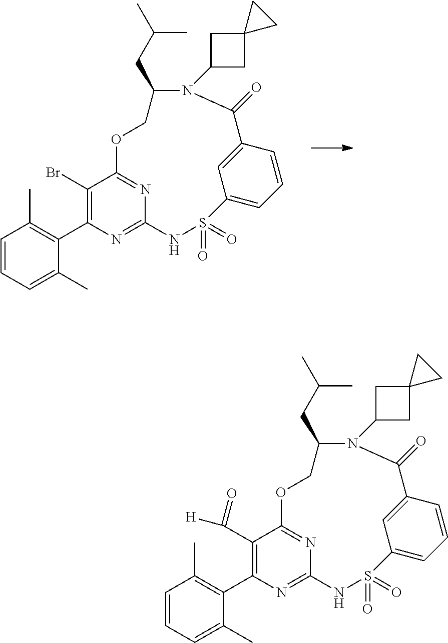 [SMILES: Cc1cccc(C)c1-c1nc2nc(c1Br)OC[C@@H](CC(C)C)N(C1CC3(CC3)C1)C(=O)c1cccc(c1)S(=O)(=O)N2 ; Cc1cccc(C)c1-c1nc2nc(c1C=O)OC[C@@H](CC(C)C)N(C1CC3(CC3)C1)C(=O)c1cccc(c1)S(=O)(=O)N2]

To a solution of (11R)-7-bromo-6-(2,6-dimethylphenyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (757 mg, 1.1835 mmol) in anhydrous THF (13 mL) at −78° C. was added n-BuLi (1.3 mL of 2.5 M, 3.2500 mmol) in hexane dropwise. The reaction solution was stirred at this temperature for 30 minutes, then methyl formate (487.00 mg, 0.5 mL, 8.1096 mmol) was added dropwise and reaction was stirred at this temperature for 1 hour. The reaction was quenched with saturated ammonium chloride (30 mL) and extracted with ethyl acetate (3×50 mL). The combined organic layers were dried over anhydrous sodium sulfate to afford crude product. The crude product obtained was purified by flash chromatography (loaded in DCM) (40 g silica gel, eluting 0 to 50% ethyl acetate in hexane) to (11R)-6-(2,6-dimethylphenyl)-11-isobutyl-2,2,13-trioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaene-7-carbaldehyde (301 mg, 43%) as an off-white solid. ESI-MS m/z calc. 588.24066, found 589.3 (M+1)⁺; Retention time: 6.35 minutes; LC method S.

### Step 2: (11R)-6-(2,6-Dimethylphenyl)-7-(hydroxymethyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 170)

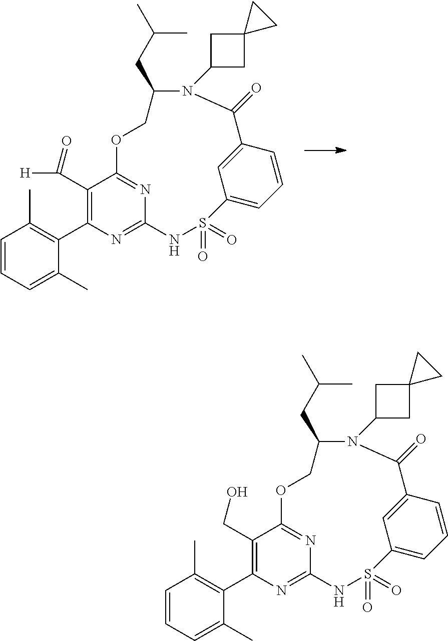 [SMILES: Cc1cccc(C)c1-c1nc2nc(c1C=O)OC[C@@H](CC(C)C)N(C1CC3(CC3)C1)C(=O)c1cccc(c1)S(=O)(=O)N2 ; Cc1cccc(C)c1-c1nc2nc(c1CO)OC[C@@H](CC(C)C)N(C1CC3(CC3)C1)C(=O)c1cccc(c1)S(=O)(=O)N2]

To a solution of (11R)-6-(2,6-dimethylphenyl)-11-isobutyl-2,2,13-trioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaene-7-carbaldehyde (35.6 mg, 0.0605 mmol) in MeOH (3 mL) was added sodium borohydride (15 mg, 0.3965 mmol). The reaction was stirred at ambient temperature for 30 minutes, then aqueous HCl solution (1 mL, 1N) and water (10 mL) were added. The solution was extracted with ethyl acetate (3×20 mL). The combined organic layers were washed with brine (3×5 mL), dried over anhydrous sodium sulfate, filtered and concentrated under reduced pressure. The crude product obtained was purified by reverse HPLC (20 to 80% mobile B; mobile A: 0.1% TFA in water; mobile B: 0.1% TFA in acetonitrile). Pure fractions were combined and lyophilized to afford (11R)-6-(2,6-dimethylphenyl)-7-(hydroxymethyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (26.8 mg, 73%) as a white solid. ESI-MS m/z calc. 590.2563, found 591.5 (M+1)⁺; Retention time: 2.43 minutes; LC method W. ¹H NMR (500 MHz, DMSO-d₆) δ 8.42 (s, 1H), 7.91 (d, J=7.6 Hz, 1H), 7.67 (dd, J=16.3, 8.8 Hz, 2H), 7.27 (t, J=7.6 Hz, 1H), 7.16 (d, J=7.7 Hz, 1H), 7.11 (d, J=7.6 Hz, 1H), 5.19 (dd, J=10.5, 4.3 Hz, 1H), 4.43 (t, J=11.1 Hz, 2H), 4.24 (p, J=8.5 Hz, 1H), 3.94-3.81 (m, 2H), 3.73 (ddt, J=11.7, 7.0, 3.7 Hz, 1H), 3.25 (t, J=9.3 Hz, 2H), 2.18-2.09 (m, 2H), 2.07 (s, 3H), 1.84 (s, 3H), 1.65 (t, J=12.4 Hz, 1H), 1.41-1.23 (m, 2H), 0.73 (d, J=6.4 Hz, 3H), 0.53 (dd, J=9.4, 6.7 Hz, 2H), 0.49-0.43 (m, 2H), 0.19 (d, J=6.1 Hz, 3H).

### Example 65: Preparation of Compound 171

### Step 1: (11R)-7-[(Dimethylamino)methyl]-6-(2,6-dimethylphenyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 171)

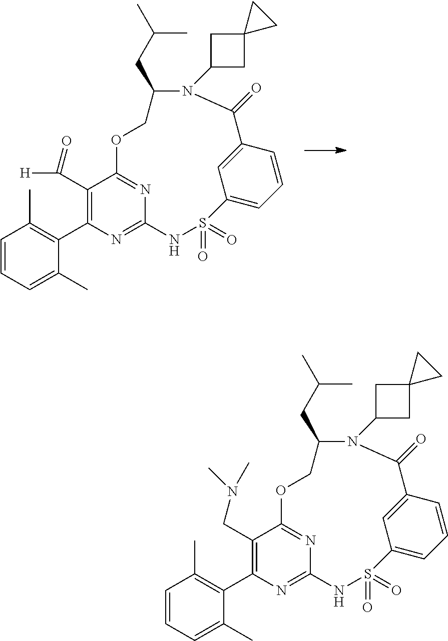 [SMILES: Cc1cccc(C)c1-c1nc2nc(c1C=O)OC[C@@H](CC(C)C)N(C1CC3(CC3)C1)C(=O)c1cccc(c1)S(=O)(=O)N2 ; Cc1cccc(C)c1-c1nc2nc(c1CN(C)C)OC[C@@H](CC(C)C)N(C1CC3(CC3)C1)C(=O)c1cccc(c1)S(=O)(=O)N2]

To a solution of (11R)-6-(2,6-dimethylphenyl)-11-isobutyl-2,2,13-trioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaene-7-carbaldehyde (26.6 mg, 0.0452 mmol) in anhydrous 1,2-dichloroethane (1 mL) were added dimethylamine hydrochloride (9.8 mg, 0.1202 mmol) and Hunig's base (15.582 mg, 0.021 mL, 0.1206 mmol) followed by Sodium triacetoxyborohydride (29 mg, 0.1368 mmol). The resulting solution was stirred at ambient temperature for 19 hours. Then saturated sodium bicarbonate aqueous solution (10 mL) was added and extracted with dichloromethane (3×20 mL). The combined organic layers were washed with brine (10 mL), dried over anhydrous sodium sulfate, filtered and concentrated. The crude product obtained was purified by reverse HPLC (15 to 75% mobile B; mobile A: 5 mM HCl in water; mobile B: acetonitrile). Pure fractions were lyophilized to afford (11R)-7-[(dimethylamino)methyl]-6-(2,6-dimethylphenyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (hydrochloride salt) (22.6 mg, 74%) as a white solid. ESI-MS m/z calc. 617.3036, found 618.8 (M+1)⁺; Retention time: 1.69 minutes; LC method W. ¹H NMR (500 MHz, DMSO-d₆) δ 9.76 (s, 1H), 8.46 (s, 1H), 7.94 (d, J=7.7 Hz, 1H), 7.71 (dd, J=18.9, 11.4 Hz, 2H), 7.34 (t, J=7.6 Hz, 1H), 7.22 (d, J=7.7 Hz, 1H), 7.17 (d, J=7.6 Hz, 1H), 6.54 (s, 1H), 5.15 (dd, J=10.6, 4.2 Hz, 1H), 4.57 (t, J=11.1 Hz, 1H), 4.20 (p, J=8.5 Hz, 1H), 3.72 (dq, J=11.5, 4.1 Hz, 1H), 3.26 (t, J=9.3 Hz, 2H), 2.71 (s, 2H), 2.43 (s, 2H), 2.17 (s, 3H), 2.11 (q, J=9.2 Hz, 2H), 1.81 (s, 3H), 1.70 (t, J=11.3 Hz, 1H), 1.29 (d, J=8.0 Hz, 2H), 0.79 (d, J=5.8 Hz, 3H), 0.54 (dd, J=9.3, 6.8 Hz, 2H), 0.47 (dq, J=8.3, 3.7, 3.1 Hz, 2H), 0.23 (d, J=5.2 Hz, 3H).

### Example 66: Preparation of Compound 172

### Step 1: (10R,11R)-10-(4-tert-Butylphenyl)-6-(2,6-dimethylphenyl)-7,11-dimethyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 172)

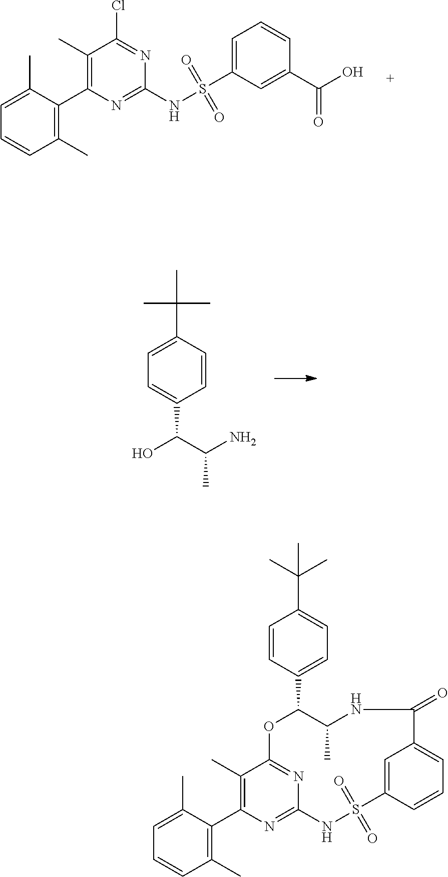 [SMILES: Cc1cccc(C)c1-c1nc(NS(=O)(=O)c2cccc(C(=O)O)c2)nc(Cl)c1C ; C[C@@H](N)[C@H](O)c1ccc(C(C)(C)C)cc1 ; Cc1cccc(C)c1-c1nc2nc(c1C)O[C@H](c1ccc(C(C)(C)C)cc1)[C@@H](C)NC(=O)c1cccc(c1)S(=O)(=O)N2]

3-[[4-Chloro-6-(2,6-dimethylphenyl)-5-methyl-pyrimidin-2-yl]sulfamoyl]benzoic acid (100.1 mg, 0.2318 mmol), (1R,2R)-2-amino-1-(4-tert-butylphenyl)propan-1-ol (49.2 mg, 0.2326 mmol), and sodium tert-butoxide (97.8 mg, 1.018 mmol) were combined in THF (1 mL) and stirred at room temperature for 4 h. At this point the reaction mixture was added to a stirred solution of HATU (175.6 mg, 0.4618 mmol) in DMF (2 mL) and the reaction was stirred an additional 1 h. The reaction was filtered and purified by reverse phase HPLC utilizing a gradient of 1-99% acetonitrile in 5 mM aqueous HCl to yield (10R,11R)-10-(4-tert-butylphenyl)-6-(2,6-dimethylphenyl)-7,11-dimethyl-2,2-dioxo-9-oxa-2λ⁶-thia-3,5,12,19-tetrazatricyclo[12.3.1.14,8]nonadeca-1(18),4(19),5,7,14,16-hexaen-13-one (11.4 mg, 8%). ESI-MS m/z calc. 584.2457, found 585.4 (M+1)⁺; Retention time: 2.85 minutes; LCMS LC method I.

### Example 67: Preparation of Compound 173

### Step 1: tert-Butyl N-tert-butoxycarbonyl-N-[4-chloro-6-(2-isopropylphenyl)-5-methyl-pyrimidin-2-yl]carbamate

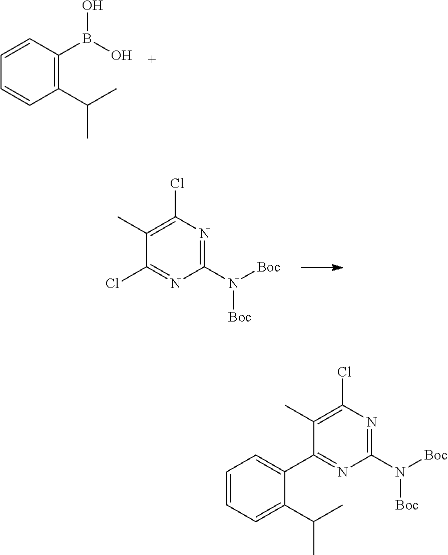 [SMILES: CC(C)c1ccccc1B(O)O ; Cc1c(Cl)nc(N(C(=O)OC(C)(C)C)C(=O)OC(C)(C)C)nc1Cl ; Cc1c(Cl)nc(N(C(=O)OC(C)(C)C)C(=O)OC(C)(C)C)nc1-c1ccccc1C(C)C]

A mixture of tert-butyl N-tert-butoxycarbonyl-N-(4,6-dichloro-5-methyl-pyrimidin-2-yl)carbamate (7.8 g, 20.621 mmol), (2-isopropylphenyl)boronic acid (3 g, 18.292 mmol), cesium carbonate (15 g, 46.038 mmol) was degassed for 30 min. Pd(dppf)Cl₂ (1.5 g, 1.8368 mmol) was then added and the mixture was heated to 80° C. under nitrogen overnight. The mixture was then quenched with DI water (50 mL) and EtOAc (100 mL). The aqueous layer was extracted with EtOAc (2×100 mL). The organic layers were combined, washed with brine, dried with anhydrous sodium sulfate, filtered, and concentrated under reduced pressure. The crude was purified via silica gel column chromatography (eluting 0 to 40% EtOAc in Hexanes) yielding tert-butyl N-tert-butoxycarbonyl-N-[4-chloro-6-(2-isopropylphenyl)-5-methyl-pyrimidin-2-yl]carbamate (5.8 g, 65%) as a sticky solid. ¹H NMR (500 MHz, Chloroform-d) δ 7.44 (dd, J=3.8, 1.1 Hz, 2H), 7.30-7.25 (m, 1H), 7.06 (dd, J=7.5, 1.0 Hz, 1H), 2.71-2.61 (m, 1H), 2.20 (s, 3H), 1.44 (s, 18H), 1.16 (d, J=6.7 Hz, 6H). ESI-MS m/z calc. 461.20813, found 462.5 (M+1)⁺; Retention time: 3.72 minutes; LC method T.

### Step 2: 4-Chloro-6-(2-isopropylphenyl)-5-methyl-pyrimidin-2-amine

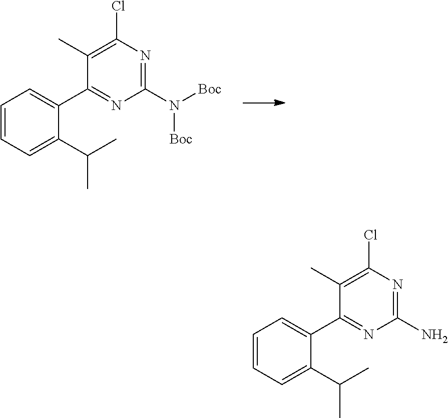 [SMILES: Cc1c(Cl)nc(N(C(=O)OC(C)(C)C)C(=O)OC(C)(C)C)nc1-c1ccccc1C(C)C ; Cc1c(Cl)nc(N)nc1-c1ccccc1C(C)C]

To a solution of tert-butyl N-tert-butoxycarbonyl-N-[4-chloro-6-(2-isopropylphenyl)-5-methyl-pyrimidin-2-yl]carbamate (5.8 g, 11.927 mmol) in anhydrous DCM (40 mL) was added HCl in Dioxane (10 mL of 4 M, 40.000 mmol). The solution was stirred at room temperature for 3 h. LCMS showed incomplete deprotection. Additional HCl in Dioxane (30 mL of 4 M, 120.00 mmol) was added and stirring was continued for 12 h. The solvents were removed by rotary evaporation and the oily residue was dried in vacuo for 1 h. It was then triturated with diethyl ether (2×100 mL) yielding 4-chloro-6-(2-isopropylphenyl)-5-methyl-pyrimidin-2-amine (hydrochloride salt) (3.44 g, 95%) as an off-white solid. This was used in the next step without further purification. ESI-MS m/z calc. 261.10327, found 262.3 (M+1)⁺; Retention time: 2.67 minutes; LC method T.

### Step 3: Methyl 3-[[4-chloro-6-(2-isopropylphenyl)-5-methyl-pyrimidin-2-yl]sulfamoyl]benzoate

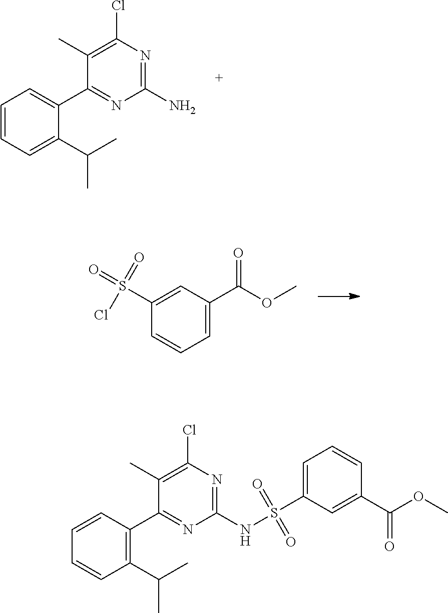 [SMILES: Cc1c(Cl)nc(N)nc1-c1ccccc1C(C)C ; COC(=O)c1cccc(S(=O)(=O)Cl)c1 ; COC(=O)c1cccc(S(=O)(=O)Nc2nc(Cl)c(C)c(-c3ccccc3C(C)C)n2)c1]

4-Chloro-6-(2-isopropylphenyl)-5-methyl-pyrimidin-2-amine (hydrochloride salt) (3.12 g, 10.253 mmol) was dissolved in THF (50 mL) and the mixture was cooled in an ice bath under stirring and nitrogen. To the cold solution, methyl 3-chlorosulfonylbenzoate (3.62 g, 15.427 mmol) in solution in THF (30 mL) was added. At 0° C., Lithium tert-pentoxide in heptane (7.5921 g, 10.400 mL of 40% w/w, 32.2789 mmol) was added dropwise and the reaction mixture was stirred at room temperature for 1 h. The reaction mixture was cooled to 0° C. Additional methyl 3-chlorosulfonylbenzoate (3.62 g, 15.427 mmol) and lithium tert-pentoxide in heptane (7.5921 g, 10.400 mL of 40% w/w, 32.2789 mmol) were added at 0° C. The reaction mixture was warmed up and stirred at room temperature for 1 h. Another reaction was combined (0.300 g, 0.9859 mmol) with this batch and the reaction was quenched with HCl 1 N (50 mL). The reaction was diluted with water (100 mL). The organic phase was isolated, and the aqueous phase was extracted with EtOAc (3×100 mL). The organic phase was dried over anhydrous sodium sulfate, filtered, and concentrated. The crude was purified by flash chromatography (silica gel 120 g, dry loading in silica gel, 0-40% acetone in hexane) to give methyl 3-[[4-chloro-6-(2-isopropylphenyl)-5-methyl-pyrimidin-2-yl]sulfamoyl]benzoate (4.47 g, 95%) as a white foam. ESI-MS m/z calc. 459.10196, found 460.3 (M+1)⁺; Retention time: 3.21 minutes; LC method T. ¹H NMR (500 MHz, cdcl3) δ 8.76 (s, 1H), 8.32-8.26 (m, 1H), 8.24-8.19 (m, 1H), 7.98 (s, 1H), 7.52 (t, J=7.9 Hz, 1H), 7.44-7.36 (m, 2H), 7.24-7.18 (m, 1H), 6.89 (d, J=8.1 Hz, 1H), 3.88 (s, 3H), 2.56 (hept, J=13.7, 6.8 Hz, 1H), 1.99 (s, 3H), 1.08 (d, J=31.1 Hz, 6H).

### Step 4: 3-[[4-Chloro-6-(2-isopropylphenyl)-5-methyl-pyrimidin-2-yl]sulfamoyl]benzoic acid

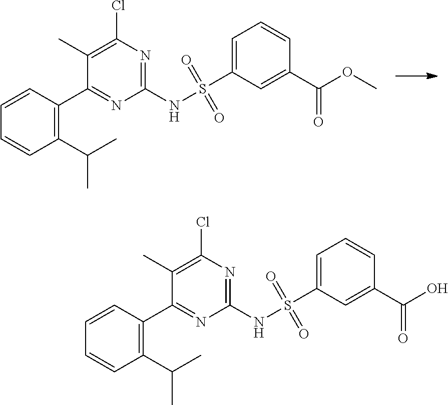 [SMILES: COC(=O)c1cccc(S(=O)(=O)Nc2nc(Cl)c(C)c(-c3ccccc3C(C)C)n2)c1 ; Cc1c(Cl)nc(NS(=O)(=O)c2cccc(C(=O)O)c2)nc1-c1ccccc1C(C)C]

Methyl 3-[[4-chloro-6-(2-isopropylphenyl)-5-methyl-pyrimidin-2-yl]sulfamoyl]benzoate (4.47 g, 9.7185 mmol) in THF (40 mL) was added aq. NaOH (25 mL of 3 M, 75.000 mmol). The reaction mixture was stirred at room temperature for 2 h. After completion, the crude was combined with another reaction (run on 0.47 g, 1.0219 mmol) and aqueous HCl (1 M) was added to acidify the solution (pH=3). The aqueous phase was extracted with EtOAc (3×100 mL). The combined organic layers were dried over anhydrous sodium sulfate and concentrated in vacuo to afford 3-[[4-chloro-6-(2-isopropylphenyl)-5-methyl-pyrimidin-2-yl]sulfamoyl]benzoic acid (4.6 g, 101% corrected yield) as white solids. ¹H NMR (500 MHz, CDCl₃) δ 8.86 (s, 1H), 8.40 (d, J=7.9 Hz, 1H), 8.26 (d, J=7.8 Hz, 1H), 7.58 (t, J=7.8 Hz, 1H), 7.46-7.40 (m, 2H), 7.28-7.21 (m, 2H), 6.93 (d, J=7.7 Hz, 1H), 4.13 (q, J=7.1 Hz, 1H), 2.57 (hept, J=13.6, 6.8 Hz, 1H), 2.03 (s, 3H), 1.13 (d, J=6.8 Hz, 3H), 1.09 (d, J=6.7 Hz, 3H). ESI-MS m/z calc. 445.0863, found 446.5 (M+1)⁺; Retention time: 2.87 minutes; LC method T.

### Step 5: 3-[[4-[(2R)-2-Amino-4,4-dimethyl-pentoxy]-6-(2-isopropylphenyl)-5-methyl-pyrimidin-2-yl]sulfamoyl]benzoic acid

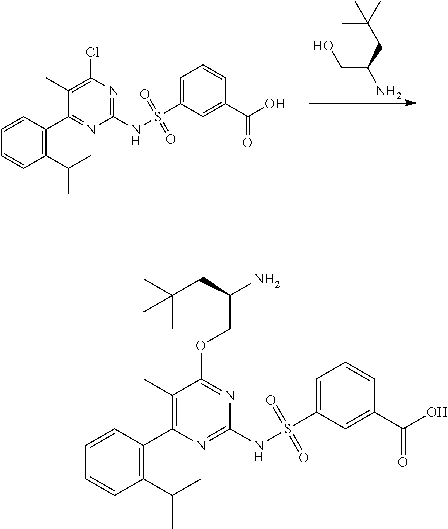

To a cloudy solution of 3-[[4-chloro-6-(2-isopropylphenyl)-5-methyl-pyrimidin-2-yl]sulfamoyl]benzoic acid (2.4 g, 5.1130 mmol) and [(1R)-1-(hydroxymethyl)-3,3-dimethyl-butyl]amine (hydrochloride salt) (1 g, 5.8446 mmol) in THF (40 mL) maintained at 5° C. with an ice-water bath, was added NaOtBu (2.5 g, 26.014 mmol) and the mixture was stirred at room temperature for 3 h. The reaction was added 1N HCl (50 mL) and extracted with EtOAc (3×100 mL). The organic layers were combined, dried and concentrated. The crude was purified by HPLC (Mobile phase A: water buffered with 5 mM HCl; Mobile phase B: acetonitrile; gradient: 35% B to 75% B, 60 mL/min) to afford 3-[[4-[(2R)-2-amino-4,4-dimethyl-pentoxy]-6-(2-isopropylphenyl)-5-methyl-pyrimidin-2-yl]sulfamoyl]benzoic acid (hydrochloride salt) (2.4548 g, 81%) as a white solid. ¹H NMR (500 MHz, DMSO) δ 8.50 (s, 1H), 8.12 (d, J=7.7 Hz, 1H), 8.08 (d, J=7.7 Hz, 1H), 7.67 (t, J=7.8 Hz, 1H), 7.45 (d, J=3.8 Hz, 2H), 7.30-7.23 (m, 1H), 7.08 (s, 1H), 4.39 (d, J=11.0 Hz, 1H), 4.20 (s, 1H), 4.08 (s, 1H), 3.59 (s, 2H), 2.61-2.56 (m, 1H), 2.53 (s, 3H), 1.70 (s, 3H), 1.68-1.59 (m, 1H), 1.53 (dd, J=14.5, 4.0 Hz, 1H), 1.08 (d, J 4.7 Hz, 6H), 0.94 (s, 9H). ESI-MS m/z calc. 540.24066, found 541.6 (M+1)⁺; Retention time: 1.99 minutes; LC method W.

### Step 6: (11R)-11-(2,2-Dimethylpropyl)-7-methyl-12-{[5-(morpholin-4-yl)pyrimidin-2-yl]methyl}-6-[2-(propan-2-yl)phenyl]-9-oxa-2λ⁶-thia-3,5,12,19-tetraazatricyclo[12.3.1.14,8]nonadeca-1(17),4(19),5,7,14(18),15-hexaene-2,2,13-trione (Compound 173)

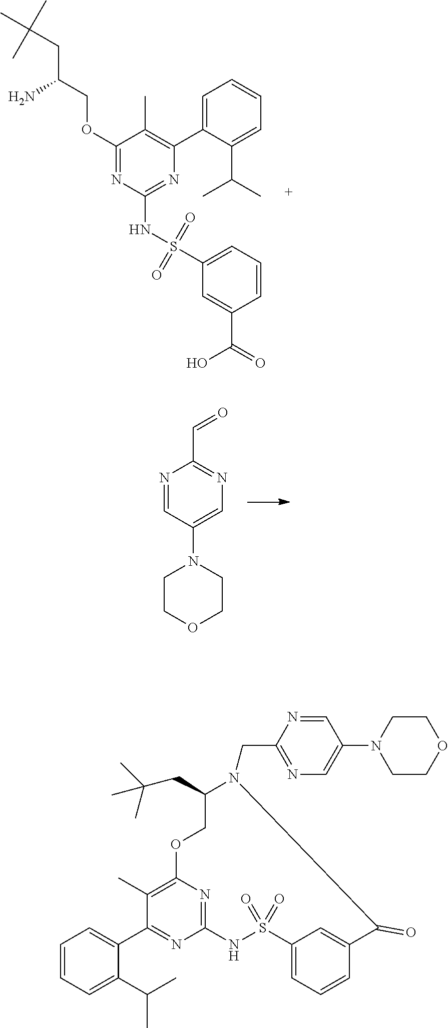 [SMILES: Cc1c(OC[C@H](N)CC(C)(C)C)nc(NS(=O)(=O)c2cccc(C(=O)O)c2)nc1-c1ccccc1C(C)C ; O=Cc1ncc(N2CCOCC2)cn1 ; Cc1c2nc(nc1-c1ccccc1C(C)C)NS(=O)(=O)c1cccc(c1)C(=O)N(Cc1ncc(N3CCOCC3)cn1)[C@H](CC(C)(C)C)CO2]

A 4 mL vial was charged with 3-[[4-[(2R)-2-amino-4,4-dimethyl-pentoxy]-6-(2-isopropylphenyl)-5-methyl-pyrimidin-2-yl]sulfamoyl]benzoic acid (hydrochloride salt) (50 mg, 0.08663 mmol), 5-morpholinopyrimidine-2-carbaldehyde (hydrochloride salt) (24 mg, 0.1045 mmol) and DCM (200 μL). The solution was stirred at room temperature for 15 min. Sodium triacetoxyborohydride (35 mg, 0.1651 mmol) was added, the vial was purged with nitrogen, capped and the mixture was stirred at room temperature for 30 min. More sodium triacetoxyborohydride (72 mg, 0.3397 mmol) was added and the mixture was stirred at room temperature for 3.5 h. The solution was quenched with a minimum amount of 1N aqueous HCl. Methanol and DMSO were added. The solution was filtered and purified by reverse phase preparative HPLC (C₁₈ column) using a gradient (1-99% over 30 min) of acetonitrile in aqueous 5 mM HCl to provide 3-[[4-[(2R)-4,4-dimethyl-2-[(5-morpholinopyrimidin-2-yl)methylamino]pentoxy]-6-(2-isopropylphenyl)-5-methyl-pyrimidin-2-yl]sulfamoyl]benzoic acid (hydrochloride salt) (43.3 mg, 66%) as a tan solid. ESI-MS m/z calc. 717.3309, found 718.4 (M+1)⁺; Retention time: 1.39 minutes; LC method A.

The material was combined in a 4 mL vial with CDMT (15 mg, 0.08543 mmol) and anhydrous DMF (1 mL). The mixture was cooled down in an ice-water bath. 4-methylmorpholine (30 μL, 0.2729 mmol) was added and the mixture was stirred in the cooling bath that was allowed to warm to room temperature. After 19 h, the solution was diluted with DMSO, filtered and purified by reverse phase preparative HPLC (C₁₈ column) using a gradient (1-99% over 30 min) of acetonitrile in aqueous 5 mM HCl to provide (11R)-11-(2,2-dimethylpropyl)-7-methyl-12-{[5-(morpholin-4-yl)pyrimidin-2-yl]methyl}-6-[2-(propan-2-yl)phenyl]-9-oxa-2λ⁶-thia-3,5,12,19-tetraazatricyclo[12.3.1.14,8]nonadeca-1(17),4(19),5,7,14(18),15-hexaene-2,2,13-tri one (10.7 mg, 18%) as a tan solid. ESI-MS m/z calc. 699.3203, found 700.39 (M+1)⁺; Retention time: 1.86 minutes; LC method A. ¹H NMR (400 MHz, DMSO-d₆) mixture of atropisomers (60:40) δ 13.42-11.00 (broad m, 1H), 8.71 (s, 1H), 8.54-8.45 (m, 2H), 7.95 (s, 1H), 7.67 (s, 2H), 7.54-7.39 (m, 2H), 7.36-6.89 (m, 2H), 5.47-5.32 (m, 1H), 4.85 (d, J=16.3, 1H), 4.68-4.55 (m, 1H), 4.30-3.91 (m, 2H), 3.75 (t, J=4.8 Hz, 4H), 3.23 (t, J=4.9 Hz, 4H), 2.68 (overlapped with DMSO rotation peak, m, 0.4 H), 2.69-2.59 (m, 0.6H), 2.41-2.19 (m, 1H), 1.92-1.75 (m, 1H), 1.65 (s, 3H), 1.45-1.30 (m, 1H), 1.21-0.92 (m, 6H), 0.68-0.43 (m, 9H).

### Example 68: Characterization of Compounds 174-190

The compounds in the following tables were prepared in a manner analogous to that described above using commercially available reagents and intermediates described herein.

TABLE 13

| Compound | | LCMS Rt | Calc. | | LCMS |
|---|---|---|---|---|---|
| Number | Structure | (min) | Mass | M + 1 | Method |
| 174 | 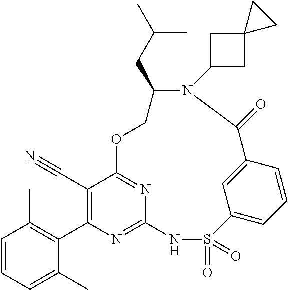 [SMILES: Cc1cccc(C)c1-c1nc2nc(c1C#N)OC[C@@H](CC(C)C)N(C1CC3(CC3)C1)C(=O)c1cccc(c1)S(=O)(=O)N2] | 3.01 | 585.241 | 586.1 | W |
| 175 | 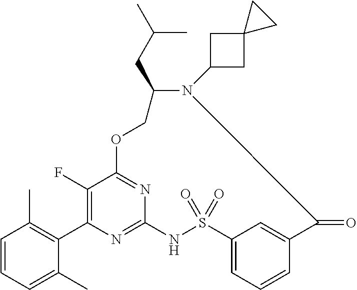 [SMILES: Cc1cccc(C)c1-c1nc2nc(c1F)OC[C@@H](CC(C)C)N(C1CC3(CC3)C1)C(=O)c1cccc(c1)S(=O)(=O)N2] | 3.36 | 578.236 | 579.4 | T |
| 176 | 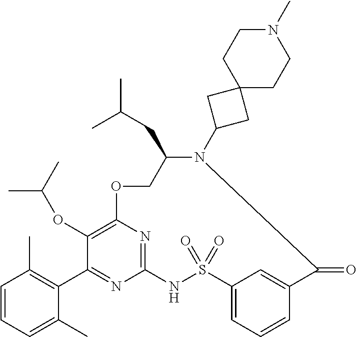 [SMILES: Cc1cccc(C)c1-c1nc2nc(c1OC(C)C)OC[C@@H](CC(C)C)N(C1CC3(CCN(C)CC3)C1)C(=O)c1cccc(c1)S(=O)(=O)N2] | 1.46 | 675.345 | 676.5 | A |
| 177 | 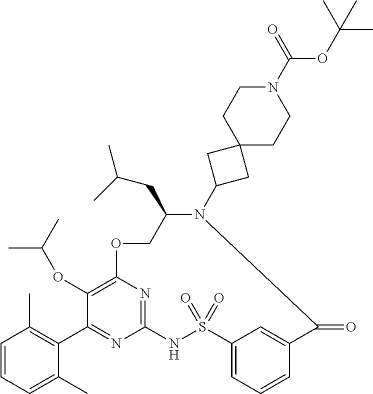 [SMILES: Cc1cccc(C)c1-c1nc2nc(c1OC(C)C)OC[C@@H](CC(C)C)N(C1CC3(CCN(C(=O)OC(C)(C)C)CC3)C1)C(=O)c1cccc(c1)S(=O)(=O)N2] | 2.39 | 761.382 | 762.5 | A |
| 178 | 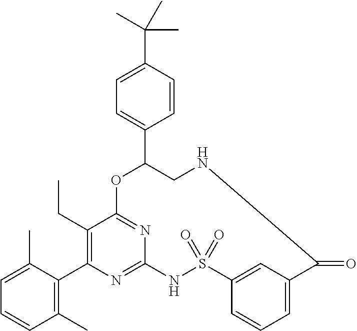 [SMILES: CCc1c2nc(nc1-c1c(C)cccc1C)NS(=O)(=O)c1cccc(c1)C(=O)NCC(c1ccc(C(C)(C)C)cc1)O2] | 1.92 | 584.246 | 585.3 | A |
| 179 | 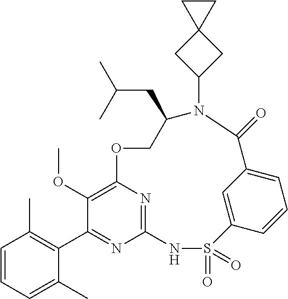 [SMILES: COc1c2nc(nc1-c1c(C)cccc1C)NS(=O)(=O)c1cccc(c1)C(=O)N(C1CC3(CC3)C1)[C@H](CC(C)C)CO2] | 2.1 | 590.256 | 591.1 | A |
| 180 | 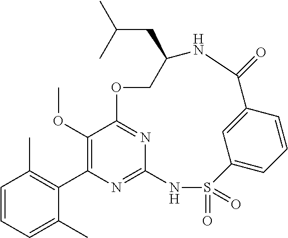 [SMILES: COc1c2nc(nc1-c1c(C)cccc1C)NS(=O)(=O)c1cccc(c1)C(=O)N[C@H](CC(C)C)CO2] | 1.53 | 510.194 | 511 | A |
| 181 | 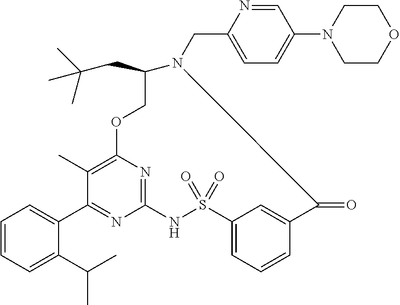 [SMILES: Cc1c2nc(nc1-c1ccccc1C(C)C)NS(=O)(=O)c1cccc(c1)C(=O)N(Cc1ccc(N3CCOCC3)cn1)[C@H](CC(C)(C)C)CO2] | 1.58 | 712.341 | 713.41 | A |
| 182 | 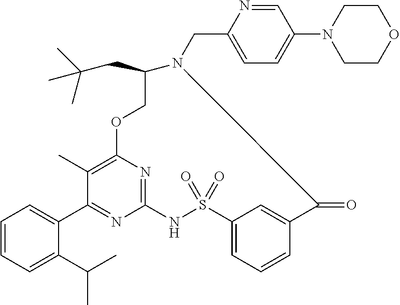 [SMILES: Cc1c2nc(nc1-c1ccccc1C(C)C)NS(=O)(=O)c1cccc(c1)C(=O)N(Cc1ccc(N3CCOCC3)cn1)[C@H](CC(C)(C)C)CO2] | 1.51 | 698.325 | 699.4 | A |
| 183 | 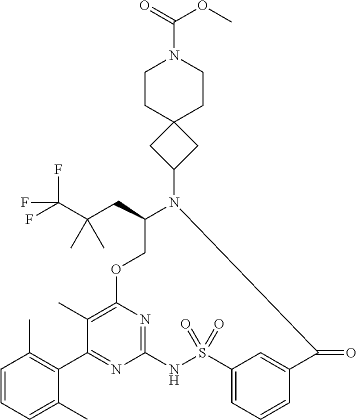 [SMILES: COC(=O)N1CCC2(CC1)CC(N1C(=O)c3cccc(c3)S(=O)(=O)Nc3nc(c(C)c(-c4c(C)cccc4C)n3)OC[C@H]1CC(C)(C)C(F)(F)F)C2] | 1.98 | 743.296 | 744.71 | A |
| 184 | 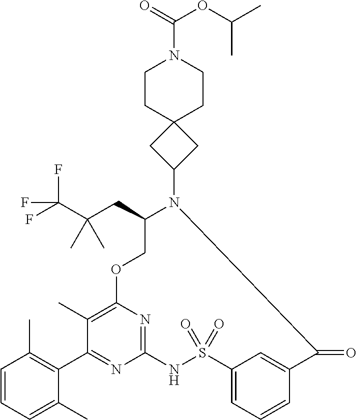 [SMILES: Cc1cccc(C)c1-c1nc2nc(c1C)OC[C@@H](CC(C)(C)C(F)(F)F)N(C1CC3(CCN(C(=O)OC(C)C)CC3)C1)C(=O)c1cccc(c1)S(=O)(=O)N2] | 2.16 | 771.328 | 772.94 | A |
| 185 | 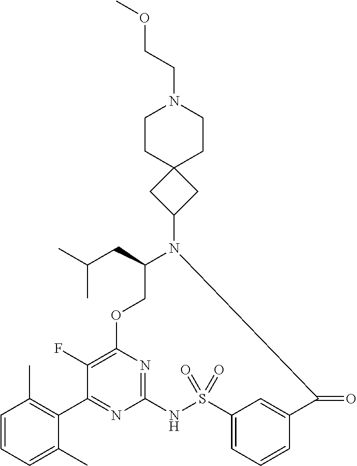 [SMILES: COCCN1CCC2(CC1)CC(N1C(=O)c3cccc(c3)S(=O)(=O)Nc3nc(c(F)c(-c4c(C)cccc4C)n3)OC[C@H]1CC(C)C)C2] | 1.47 | 679.32 | 680.84 | A |
| 186 | 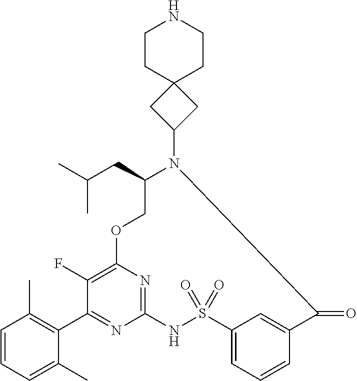 [SMILES: Cc1cccc(C)c1-c1nc2nc(c1F)OC[C@@H](CC(C)C)N(C1CC3(CCNCC3)C1)C(=O)c1cccc(c1)S(=O)(=O)N2] | 1.52 | 621.279 | 622.58 | A |
| 187 | 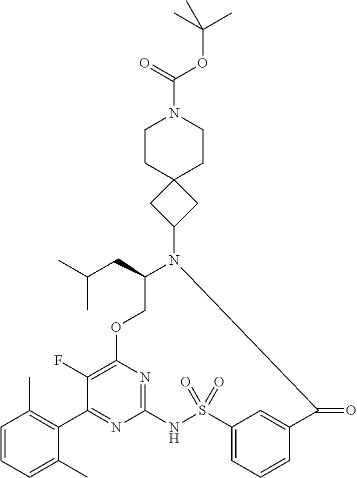 [SMILES: Cc1cccc(C)c1-c1nc2nc(c1F)OC[C@@H](CC(C)C)N(C1CC3(CCN(C(=O)OC(C)(C)C)CC3)C1)C(=O)c1cccc(c1)S(=O)(=O)N2] | 2.39 | 721.331 | 722.49 | A |
| 188 | 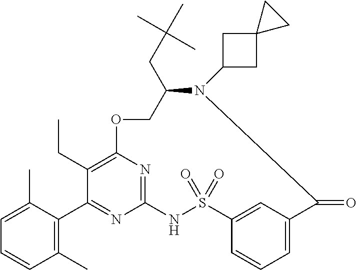 [SMILES: CCc1c2nc(nc1-c1c(C)cccc1C)NS(=O)(=O)c1cccc(c1)C(=O)N(C1CC3(CC3)C1)[C@H](CC(C)(C)C)CO2] | 2.2 | 602.293 | 603.5 | A |
| 116 | 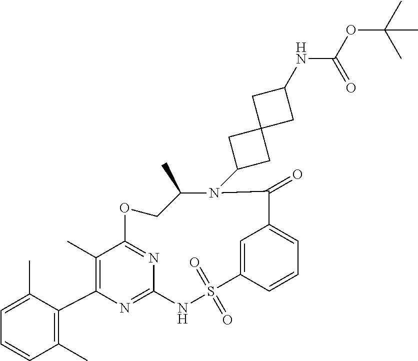 [SMILES: Cc1cccc(C)c1-c1nc2nc(c1C)OC[C@@H](C)N(C1CC3(CC(NC(=O)OC(C)(C)C)C3)C1)C(=O)c1cccc(c1)S(=O)(=O)N2] | 1.88 | 661.293 | 662.9 | A |
| 189 | 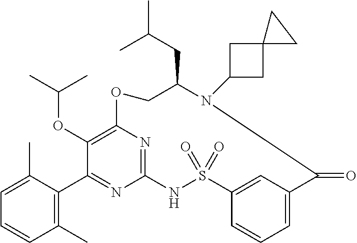 [SMILES: Cc1cccc(C)c1-c1nc2nc(c1OC(C)C)OC[C@@H](CC(C)C)N(C1CC3(CC3)C1)C(=O)c1cccc(c1)S(=O)(=O)N2] | 2.24 | 618.288 | 619.3 | A |
| 190 | 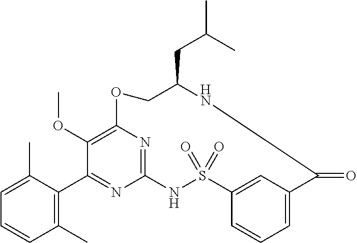 [SMILES: COc1c2nc(nc1-c1c(C)cccc1C)NS(=O)(=O)c1cccc(c1)C(=O)N[C@H](CC(C)C)CO2] | 1.59 | 508.214 | 509.3 | A |

TABLE 14

| Compound | |
|---|---|
| Number | NMR |
| 174 | ¹H NMR (500 MHz, DMSO-d₆) δ 8.46 (d, J = 2.0 Hz, 1H), 8.01 (d, J = 7.8 Hz, |
| | 1H), 7.84 (d, J = 7.6 Hz, 1H), 7.77 (t, J = 7.7 Hz, 1H), 7.31 (t, J = 7.6 Hz, 1H), |
| | 7.19 (d, J = 7.6 Hz, 1H), 7.15 (d, J = 7.6 Hz, 1H), 5.23 (dd, J = 10.8, 4.2 Hz, 1H), |
| | 4.59 (t, J = 11.2 Hz, 1H), 4.28 (p, J = 8.5 Hz, 1H), 3.65 (ddt, J = 11.2, 7.6, 3.4 |
| | Hz, 1H), 3.29-3.21 (m, 2H), 2.15-2.09 (m, 2H), 2.12 (s, 3H), 1.88 (s, 3H), 1.75 |
| | (ddd, J = 14.0, 10.6, 3.0 Hz, 1H), 1.32 (dtt, J = 11.8, 8.3, 4.4 Hz, 1H), 1.17 (ddd, |
| | J = 13.7, 10.3, 2.8 Hz, 1H), 0.74 (d, J = 6.7 Hz, 3H), 0.56-0.50 (m, 2H), 0.50- |
| | 0.42 (m, 2H), 0.25 (d, J = 6.4 Hz, 3H). |
| 175 | ¹H NMR (500 MHz, DMSO-d₆) δ 12.13 (s, 1H), 8.46 (s, 1H), 7.99 (dt, J = 7.8, |
| | 1.6 Hz, 1H), 7.83 (dt, J = 7.7, 1.5 Hz, 1H), 7.77 (t, J = 7.7 Hz, 1H), 7.28 (t, J = |
| | 7.6 Hz, 1H), 7.17 (d, J = 7.6 Hz, 1H), 7.12 (d, J = 7.6 Hz, 1H), 5.14 (dd, J = 10.8, |
| | 4.3 Hz, 1H), 4.55 (t, J = 11.2 Hz, 1H), 4.26 (p, J = 8.6 Hz, 1H), 3.71 (td, J = 10.8, |
| | 5.4 Hz, 1H), 3.31-3.23 (m, 2H), 2.20-2.04 (m, 5H), 1.86 (s, 3H), 1.71 (ddd, J = |
| | 14.0, 10.7, 2.9 Hz, 1H), 1.30 (dtt, J = 12.9, 9.3, 4.7 Hz, 1H), 1.16 (ddd, J = 13.7, |
| | 10.4, 2.8 Hz, 1H), 0.73 (d, J = 6.7 Hz, 3H), 0.57-0.42 (m, 4H), 0.21 (d, J = 6.4 |
| | Hz, 3H). |
| 181 | ¹H NMR (400 MHz, DMSO-d₆) two atropisomers detected (2:1 ratio) δ 13.91- |
| | 11.37 (broad m, 1H), 8.69-8.52 (m, 1H), 8.32 (s, 1H), 7.97 (br s, 1H), 7.85-7.53 |
| | (m, 5H), 7.47-7.36 (m, 1H), 7.33-7.19 (m, 1H), 7.11-6.99 (major atropisomer, |
| | m, 0.6H), 6.76 (minor atropisomer, s, 0.3H), 5.38-5.23 (m, 1H), 4.94-4.77 (m, |
| | 1H), 4.73-4.53 (m, 1H), 4.44-4.25 (m, 1H), 4.12-3.92 (m, 1H), 3.76 |
| | (overlapped with water, t, J = 4.8 Hz, 4H), 3.28 (overlapped with water, t, J = 4.8 |
| | Hz, 4H), 1.94-1.75 (m, 1H), 1.67 (s, 3H), 1.44-1.29 (m, 1H), 1.21 (minor |
| | atropisomer, s, 3H), 1.04 (major atropisomer, s, 6H), 0.58 (minor atropisomer, s, |
| | 3H), 0.51 (major atropisomer, s, 6H). |
| 182 | ¹H NMR (400 MHz, DMSO-d₆) mixture of atropisomers (60:40) δ 13.76-11.73 |
| | (m, 1H), 8.69-8.52 (m, 1H), 8.35-8.26 (m, 1H), 8.03-7.88 (m, 1H), 7.79-7.52 |
| | (m, 4H), 7.52-7.39 (m, 2H), 7.36-6.89 (m, 2H), 5.35-5.24 (m, 1H), 4.93-4.79 |
| | (m, 1H), 4.70-4.51 (m, 1H), 4.46-4.23 (m, 1H), 4.15-3.90 (m, 1H), 3.76 (t, |
| | J = 4.8 Hz, 4H), 3.26 (t, J = 4.8 Hz, 4H), 2.68-2.59 (overlapped with DMSO |
| | rotation peak, m, 0.4H), 2.31-2.22 (m, 0.6H), 1.91-1.77 (m, 1H), 1.66 (s, 3H), |
| | 1.42-1.32 (m, 1H), 1.21-0.92 (overlapped with residual hexanes, m, 6H), 0.67- |
| | 0.39 (m, 9H). |
| 185 | ¹H NMR (500 MHz, DMSO-d₆) δ 12.14 (broad s, 1H), 10.31 (br s, 0.5 H), 10.27 |
| | (br s, 0.5 H), 8.44 (q, J = 2.0 Hz, 1H), 7.99 (dt, J = 7.7, 1.6 Hz, 1H), 7.84-7.71 |
| | (m, 2H), 7.27 (t, J = 7.6 Hz, 1H), 7.16 (d, J = 7.7 Hz, 1H), 7.11 (d, J = 7.6 Hz, |
| | 1H), 5.11 (dd, J = 10.5, 4.0 Hz, 1H), 4.51 (q, J = 10.7 Hz, 1H), 4.23-3.98 (m, |
| | 1H), 3.79-3.64 (m, 3H), 3.47-3.37 (m, 2H overlapped with water), 3.31 (s, 3H), |
| | 3.27-3.20 (m, 2H), 3.04-2.93 (m, 1H), 2.87 (q, J = 10.6 Hz, 2H), 2.78 (t, J = |
| | 9.5 Hz, 1H), 2.23-2.10 (m, 1H), 2.09 (s, 3H), 2.08-1.87 (m, 5H), 1.85 (s, 3H), |
| | 1.67 (q, J = 12.4, 12.0 Hz, 1H), 1.33-1.24 (m, 1H), 1.12 (t, J = 12.2 Hz, 1H), |
| | 0.76-0.74 (m, 3H), 0.19-0.17 (m, 3H). protonation of amine results into 2 visible |
| | isomeric forms. |
| 186 | ¹H NMR (500 MHz, DMSO-d₆) δ 12.14 (broad s, 1H), 8.94 (br s, 1H), 8.90 (br s, |
| | 1H), 8.44 (s, 1H), 7.99 (dt, J = 7.6, 1.6 Hz, 1H), 7.83-7.69 (m, 2H), 7.26 (t, J = |
| | 7.6 Hz, 1H), 7.16 (d, J = 7.6 Hz, 1H), 7.11 (d, J = 7.6 Hz, 1H), 5.10 (dd, J = 10.8, |
| | 4.4 Hz, 1H), 4.51 (t, J = 11.2 Hz, 1H), 4.09 (p, J = 8.9 Hz, 1H), 3.70 (td, J = 11.1, |
| | 5.4 Hz, 1H), 3.02 (br s, 2H), 2.95 (s, br 2H), 2.83 (q, J = 9.6 Hz, 2H), 2.15-2.00 |
| | (m, 5H), 1.94-1.79 (m, 7H), 1.67 (td, J = 11.4, 10.9, 5.5 Hz, 1H), 1.32-1.25 (m, |
| | 1H), 1.12 (ddd, J = 13.4, 10.2, 2.7 Hz, 1H), 0.73 (d, J = 6.6 Hz, 3H), 0.18 (d, J = |
| | 6.4 Hz, 3H). |

### Example 69: Characterization of Compounds 191-205

The compounds in the following tables were prepared in a manner analogous to that described above using commercially available reagents and intermediates described herein.

TABLE 15

| Compound | | LCMS | Calc. | | LCMS |
|---|---|---|---|---|---|
| number | Structure | Rt (min) | Mass | M + 1 | Method |
| 191 | 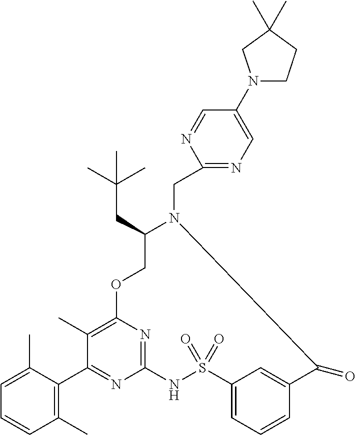 [SMILES: Cc1cccc(C)c1-c1nc2nc(c1C)OC[C@@H](CC(C)(C)C)N(Cc1ncc(N3CCC(C)(C)C3)cn1)C(=O)c1cccc(c1)S(=O)(=O)N2] | 1.76 | 697.341 | 698.6 | A |
| 192 | 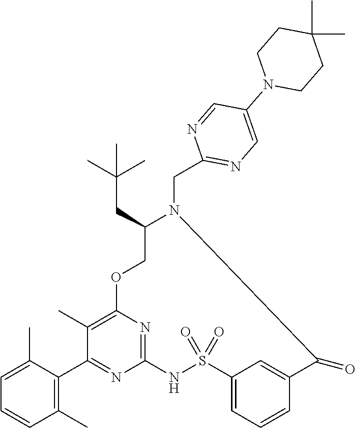 [SMILES: Cc1cccc(C)c1-c1nc2nc(c1C)OC[C@@H](CC(C)(C)C)N(Cc1ncc(N3CCC(C)(C)CC3)cn1)C(=O)c1cccc(c1)S(=O)(=O)N2] | 1.85 | 711.357 | 712.7 | A |
| 193 | 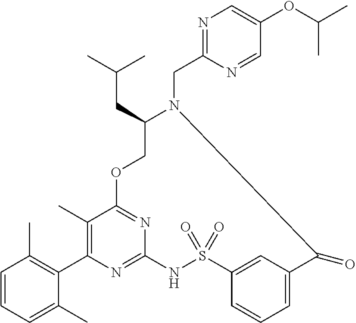 [SMILES: Cc1cccc(C)c1-c1nc2nc(c1C)OC[C@@H](CC(C)C)N(Cc1ncc(OC(C)C)cn1)C(=O)c1cccc(c1)S(=O)(=O)N2] | 1.88 | 644.278 | 645.5 | A |
| 194 | 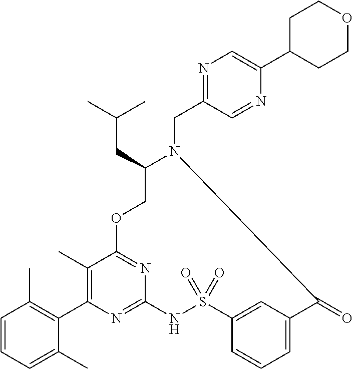 [SMILES: Cc1cccc(C)c1-c1nc2nc(c1C)OC[C@@H](CC(C)C)N(Cc1cnc(C3CCOCC3)cn1)C(=O)c1cccc(c1)S(=O)(=O)N2] | 1.75 | 670.294 | 671.7 | A |
| 195 | 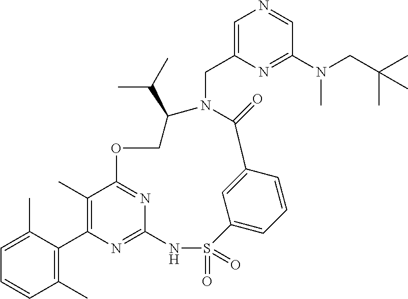 [SMILES: Cc1cccc(C)c1-c1nc2nc(c1C)OC[C@@H](C(C)C)N(Cc1cncc(N(C)CC(C)(C)C)n1)C(=O)c1cccc(c1)S(=O)(=O)N2] | 1.92 | 671.325 | 672.5 | A |
| 196 | 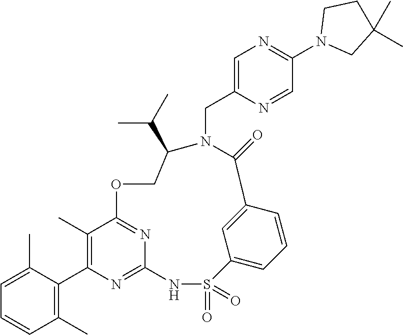 [SMILES: Cc1cccc(C)c1-c1nc2nc(c1C)OC[C@@H](C(C)C)N(Cc1cnc(N3CCC(C)(C)C3)cn1)C(=O)c1cccc(c1)S(=O)(=O)N2] | 1.92 | 669.31 | 670.5 | A |
| 197 | 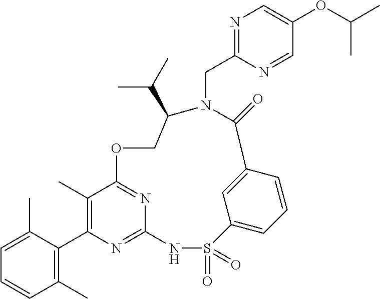 [SMILES: Cc1cccc(C)c1-c1nc2nc(c1C)OC[C@@H](C(C)C)N(Cc1ncc(OC(C)C)cn1)C(=O)c1cccc(c1)S(=O)(=O)N2] | 1.83 | 630.262 | 631.4 | A |
| 198 | 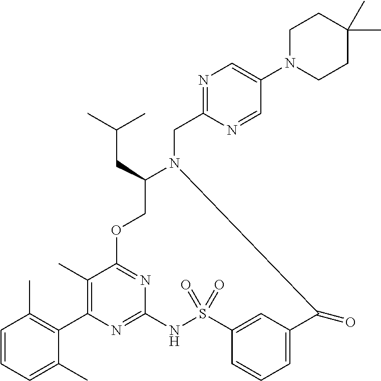 [SMILES: Cc1cccc(C)c1-c1nc2nc(c1C)OC[C@@H](CC(C)C)N(Cc1ncc(N3CCC(C)(C)CC3)cn1)C(=O)c1cccc(c1)S(=O)(=O)N2] | 1.73 | 697.341 | 698.4 | A |
| 199 | 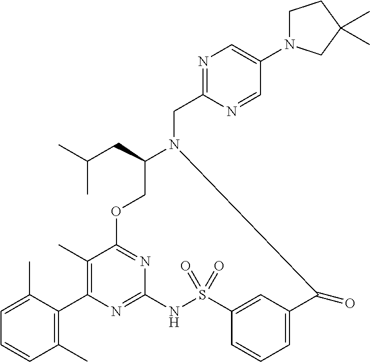 [SMILES: Cc1cccc(C)c1-c1nc2nc(c1C)OC[C@@H](CC(C)C)N(Cc1ncc(N3CCC(C)(C)C3)cn1)C(=O)c1cccc(c1)S(=O)(=O)N2] | 1.65 | 683.325 | 684.4 | A |
| 200 | 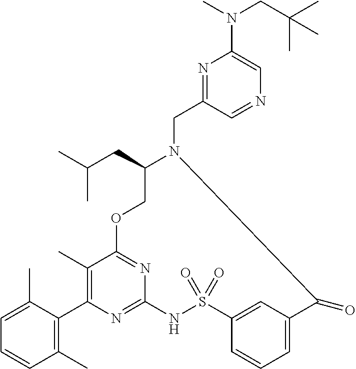 [SMILES: Cc1cccc(C)c1-c1nc2nc(c1C)OC[C@@H](CC(C)C)N(Cc1cncc(N(C)CC(C)(C)C)n1)C(=O)c1cccc(c1)S(=O)(=O)N2] | 1.65 | 685.341 | 686.5 | A |
| 201 | 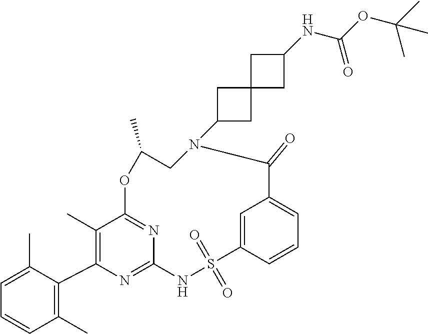 [SMILES: Cc1cccc(C)c1-c1nc2nc(c1C)O[C@H](C)CN(C1CC3(CC(NC(=O)OC(C)(C)C)C3)C1)C(=O)c1cccc(c1)S(=O)(=O)N2] | 1.83 | 661.293 | 662.6 | A |
| 202 | 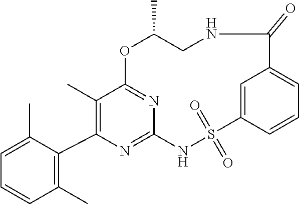 [SMILES: Cc1cccc(C)c1-c1nc2nc(c1C)O[C@H](C)CNC(=O)c1cccc(c1)S(=O)(=O)N2] | 1.28 | 452.152 | 453.1 | A |
| 203 | 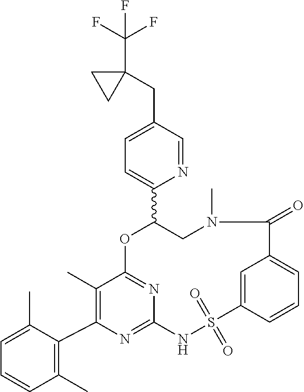 [SMILES: Cc1cccc(C)c1-c1nc2nc(c1C)OC(c1ccc(CC3(C(F)(F)F)CC3)cn1)CN(C)C(=O)c1cccc(c1)S(=O)(=O)N2] | 1.73 | 651.213 | 652.5 | A |
| 204 | 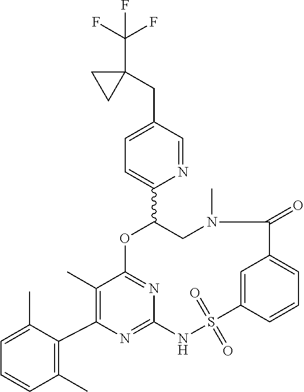 [SMILES: Cc1cccc(C)c1-c1nc2nc(c1C)OC(c1ccc(CC3(C(F)(F)F)CC3)cn1)CN(C)C(=O)c1cccc(c1)S(=O)(=O)N2] | 1.79 | 651.213 | 652.5 | A |
| 205 | 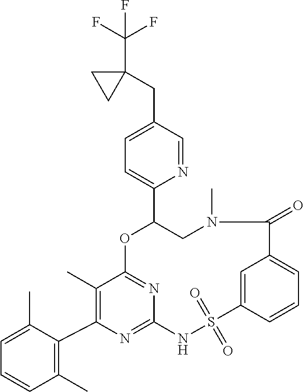 [SMILES: Cc1cccc(C)c1-c1nc2nc(c1C)OC(c1ccc(CC3(C(F)(F)F)CC3)cn1)CN(C)C(=O)c1cccc(c1)S(=O)(=O)N2] | 1.76 | 651.213 | 652.2 | A |

TABLE 16

| Compound | |
|---|---|
| number | NMR |
| 193 | ¹H NMR (400 MHz, MeOD) δ 8.87-8.75 (m, 1H), 8.51 (s, 2H), 8.01 (dt, |
| | J = 7.3, 1.7 Hz, 1H), 7.77-7.57 (m, 2H), 7.30 (t, J = 7.6 Hz, 1H), 7.18 (dd, |
| | J = 13.9, 7.6 Hz, 2H), 5.65 (dd, J = 10.5, 3.9 Hz, 1H), 5.07 (d, J = 16.5 Hz, |
| | 1H), 4.78 (hept, J = 6.1 Hz, 1H), 4.63 (d, J = 16.6 Hz, 1H), 4.26-4.18 (m, |
| | 1H), 4.17-4.08 (m, 1H), 2.12 (s, 3H), 1.95 (s, 3H), 1.84 (ddd, J = 13.9, 10.1, |
| | 3.3 Hz, 1H), 1.69 (s, 3H), 1.55-1.43 (m, 1H), 1.41-1.30 (m, 7H), 0.84 (d, |
| | J = 6.6 Hz, 3H), 0.40 (d, J = 6.4 Hz, 3H). |
| 194 | ¹H NMR (400 MHz, MeOD) δ 8.69 (d, J = 1.5 Hz, 1H), 8.67 (d, J = 1.8 Hz, |
| | 1H), 8.59 (d, J = 1.5 Hz, 1H), 8.01 (dt, J = 7.5, 1.6 Hz, 1H), 7.73-7.59 (m, |
| | 2H), 7.28 (t, J = 7.6 Hz, 1H), 7.22-7.09 (m, 2H), 5.56 (dd, J = 10.9, 4.3 Hz, |
| | 1H), 4.99 (d, J = 15.8 Hz, 1H), 4.62 (d, J = 15.7 Hz, 1H), 4.39 (t, J = 11.2 |
| | Hz, 1H), 4.21-4.11 (m, 1H), 4.11-4.03 (m, 2H), 3.64-3.52 (m, 2H), 3.16- |
| | 3.00 (m, 1H), 2.11 (s, 3H), 1.98-1.80 (m, 7H), 1.70 (s, 3H), 1.44-1.29 |
| | (m, 3H), 0.86 (d, J = 6.3 Hz, 3H), 0.36 (d, J = 6.1 Hz, 3H). |
| 195 | ¹H NMR (400 MHz, DMSO-d₆) δ 8.62 (s, 1H), 8.09 (s, 1H), 7.93-7.88 (m, |
| | 1H), 7.85 (s, 1H), 7.67 (d, J = 4.6 Hz, 2H), 7.27 (t, J = 7.6 Hz, 1H), 7.17 (d, |
| | J = 7.6 Hz, 1H), 7.13 (d, J = 7.6 Hz, 1H), 5.44 (dd, J = 11.4, 3.9 Hz, 1H), |
| | 4.86 (d, J = 15.8 Hz, 1H), 4.63 (t, J = 11.4 Hz, 1H), 4.35 (d, J = 15.7 Hz, |
| | 1H), 3.74 (ddd, J = 11.9, 8.7, 3.8 Hz, 1H), 3.48 (s, 2H), 3.16 (s, 3H), 2.27- |
| | 2.16 (m, 1H), 2.04 (s, 3H), 1.88 (s, 3H), 1.58 (s, 3H), 0.90 (s, 9H), 0.82 (d, |
| | J = 6.6 Hz, 3H), 0.78 (d, J = 6.5 Hz, 3H). (missing peak around 13 ppm) |
| 196 | ¹H NMR (400 MHz, DMSO-d₆) δ 12.88 (s, 1H), 8.66 (s, 1H), 8.08 (s, 2H), |
| | 7.90 (d, J = 6.8 Hz, 1H), 7.75-7.55 (m, 2H), 7.27 (t, J = 7.6 Hz, 1H), 7.17 |
| | (d, J = 7.6 Hz, 1H), 7.13 (d, J = 7.6 Hz, 1H), 5.44 (dd, J = 11.5, 3.9 Hz, 1H), |
| | 4.97 (d, J = 16.1 Hz, 1H), 4.53-4.37 (m, 2H), 3.70 (ddd, J = 11.9, 8.6, 3.9 |
| | Hz, 1H), 3.31-3.16 (m, 2H), 3.08 (s, 2H), 2.26-2.15 (m, 1H), 2.04 (s, 3H), |
| | 1.88 (s, 3H), 1.77 (t, J = 6.9 Hz, 2H), 1.57 (s, 3H), 1.10 (s, 6H), 0.82 (d, J = |
| | 6.7 Hz, 3H), 0.77 (d, J = 6.5 Hz, 3H). |
| 197 | ¹H NMR (400 MHz, DMSO-d₆) δ 12.93 (s, 1H), 8.66 (s, 1H), 8.51 (s, 2H), |
| | 7.91 (d, J = 6.9 Hz, 1H), 7.65 (s, 2H), 7.28 (t, J = 7.6 Hz, 1H), 7.17 (d, J = |
| | 7.6 Hz, 1H), 7.13 (d, J = 7.6 Hz, 1H), 5.45 (dd, J = 11.9, 4.1 Hz, 1H), 4.97 |
| | (d, J = 16.4 Hz, 1H), 4.81 (hept, J = 6.0 Hz, 1H), 4.58 (d, J = 16.2 Hz, 1H), |
| | 4.53 (t, J = 12.1 Hz, 1H), 3.79-3.68 (m, 1H), 2.20 (d(hep), J = 8.4, 6.5 Hz, |
| | 1H), 2.04 (s, 3H), 1.89 (s, 3H), 1.57 (s, 3H), 1.30 (two d, J = 6.0 Hz, 6H), |
| | 0.82 (d, J = 6.7 Hz, 3H), 0.77 (d, J = 6.5 Hz, 3H). |
| 203 | ¹H NMR (400 MHz, DMSO-d₆) δ 12.91 (s, 1H), 8.63 (d, J = 2.2 Hz, 2H), |
| | 7.93 (dd, J = 8.1, 2.3 Hz, 2H), 7.81 (d, J = 8.0 Hz, 1H), 7.65 (s, 2H), 7.25 |
| | (d, J = 7.9 Hz, 1H), 7.14 (d, J = 7.2 Hz, 2H), 6.47 (d, J = 10.2 Hz, 1H), 3.88- |
| | 3.70 (m, 1H), 3.49 (dd, J = 14.2, 10.9 Hz, 1H), 3.04 (s, 2H), 2.16 (s, 3H), |
| | 1.95 (d, J = 19.0 Hz, 6H), 1.58 (s, 3H), 1.07-0.95 (m, 2H), 0.93 (s, 2H).[2] |
| 204 | ¹H NMR (400 MHz, DMSO-d₆) δ 12.84 (s, 1H), 8.63 (d, J = 2.1 Hz, 2H), |
| | 8.00-7.85 (m, 2H), 7.81 (d, J = 8.0 Hz, 1H), 7.66 (d, J = 9.4 Hz, 2H), 7.26 |
| | (t, J = 7.6 Hz, 1H), 7.13 (dd, J = 7.8, 3.6 Hz, 2H), 6.47 (dd, J = 10.8, 4.4 |
| | Hz, 1H), 3.78 (dd, J = 14.3, 4.4 Hz, 1H), 3.49 (dd, J = 14.2, 10.8 Hz, 1H), |
| | 3.04 (s, 2H), 2.16 (s, 3H), 1.95 (d, J = 19.1 Hz, 6H), 1.58 (s, 3H), 1.00 (d, J |
| | 4.7 Hz, 2H), 0.94 (d, J = 11.6 Hz, 2H) |

### Example 70: Preparation of Compound 206 and Compound 207

### Step 1: Cyclohexyl(triphenyl)phosphonium bromide

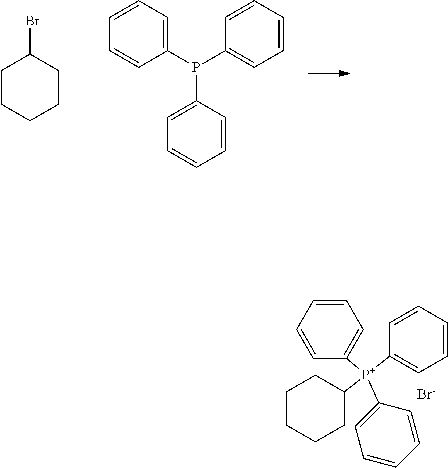 [SMILES: BrC1CCCCC1 ; c1ccc(P(c2ccccc2)c2ccccc2)cc1 ; c1ccc([P+](c2ccccc2)(c2ccccc2)C2CCCCC2)cc1 ; [Br-]]

A sealed Schlenk flask with Teflon tap was charged with triphenylphosphane (70 g, 266.89 mmol) and bromocyclohexane (52.960 g, 40 mL, 324.80 mmol) and the mixture was heated to 165° C. for 24 h. To the resulting yellow oil, toluene was added (100 mL) and the white precipitate was washed with cold THF (300 mL) and cold Et₂O (300 mL). Removal of all the volatiles and drying in vacuo yielded cyclohexyl(triphenyl)phosphonium bromide (77.2 g, 67%) as a white powder. ¹H NMR (500 MHz, Chloroform-d) δ 8.00 (dd, J=11.9, 7.7 Hz, 5H), 7.78-7.64 (m, 8H), 7.59-7.41 (m, 2H), 5.38 (t, J=12.4 Hz, 1H), 2.15 (d, J=12.3 Hz, 4H), 1.85-1.67 (m, 3H), 1.10-0.94 (m, 3H).

### Step 2: (2-Bromo-3-methyl-phenyl)methanol

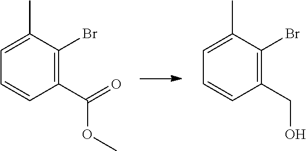

To a solution of LiBH₄ in THF (95.0 mL of 2 M, 190.0 mmol) stirring under nitrogen atmosphere and cooled in an ice-water bath was slowly added a solution of methyl 2-bromo-3-methyl-benzoate (30 g, 130.96 mmol) in ether (200 mL). Upon completion of reagent addition, the mixture was warmed to room temperature and stirred for 16 h. The mixture was quenched by the slow addition of 0.5 N HCl (50 mL) until pH=6-7. The resulting mixture was extracted with diethyl ether and the organic layer was washed with brine, dried (sodium sulfate), filtered and concentrated to give (2-bromo-3-methyl-phenyl)methanol (25.68 g, 93%); ESI-MS m/z calc. 199.9837, found 183.5 (M+1-18)⁺; Retention time: 2.53 minutes; LC Method T.

### Step 3: 2-Bromo-3-methyl-benzaldehyde

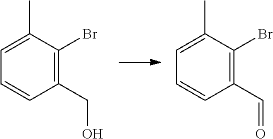

PhI(OAc)₂ (48 g, 149.02 mmol) was added to a solution of (2-bromo-3-methyl-phenyl)methanol (25.68 g, 121.34 mmol) and TEMPO (2.1 g, 13.440 mmol) in DCM (210 mL). The reaction mixture was stirred until the alcohol was no longer detectable (TLC), and then it was diluted with DCM. The mixture was washed with sat. Na₂S₂O₃ solution and extracted with DCM. The combined organic extracts were washed with aq. NaHCO₃, dried over anhydrous Na₂SO₄, and concentrated. The crude was purified by silica gel chromatography (80 g silica, 0-30% gradient of EtOAc in hexanes) to afford 2-bromo-3-methyl-benzaldehyde (22.1 g, 91%) as a white solid.

### Step 4: 2-Bromo-1-(cyclohexylidenemethyl)-3-methyl-benzene

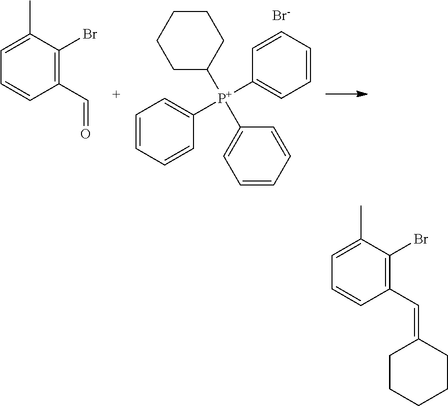

To a suspension of cyclohexyl(triphenyl)phosphonium;bromide (77.2 g, 181.50 mmol) in THF (220 mL) at 0° C. was added potassium tert-butoxide (26.165 g, 233.17 mmol) in THE (220 mL). The mixture was then stirred at RT for 1 h. The mixture was then cooled back down to 0° C. and a solution of 2-bromo-3-methyl-benzaldehyde (22.1 g, 111.03 mmol) in THE (45 mL) was added. The reaction was then stirred at RT for 17 h. The reaction mixture was then quenched with cold water (200 mL) and the aqueous layer was extracted with EtOAc (2×300 mL). The organic layers were then combined, washed with brine, dried with anhydrous Na₂SO₄, filtered, and concentrated under reduced pressure. The crude product was then purified via silica gel column chromatography (eluting with 0 to 5% EtOAc in hexanes) to yield 2-bromo-1-(cyclohexylidenemethyl)-3-methyl-benzene (24.66 g, 82%) as a clear oil. ¹H NMR (500 MHz, Chloroform-d) δ 7.17-7.06 (m, 2H), 7.03 (m, 1H), 6.19 (s, 1H), 2.43 (s, 3H), 2.33-2.25 (m, 2H), 2.24-2.16 (m, 2H), 1.72-1.64 (m, 2H), 1.64-1.58 (m, 2H), 1.55 (m, 2H).

### Step 5: 2-[2-(Cyclohexylidenemethyl)-6-methyl-phenyl]-4,4,5,5-tetramethyl-1,3,2-dioxaborolane

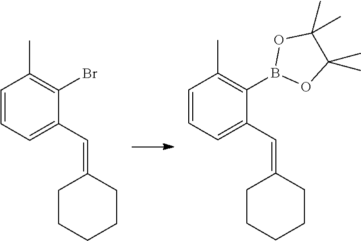 [SMILES: Cc1cccc(C=C2CCCCC2)c1Br ; Cc1cccc(C=C2CCCCC2)c1B1OC(C)(C)C(C)(C)O1]

In a 20-mL sealed tube was added 2-bromo-1-(cyclohexylidenemethyl)-3-methyl-benzene (34.9 g, 125.02 mmol) in dioxane (300 mL), and KOAc (29 g, 295.49 mmol) was added. The resulting mixture was degassed with N₂ for several minutes. Then, 4,4,5,5-tetramethyl-2-(4,4,5,5-tetramethyl-1,3,2-dioxaborolan-2-yl)-1,3,2-dioxaborolane (54 g, 212.65 mmol) was added, followed by Pd(dppf)Cl₂ (9 g, 12.3 mmol), and the reaction was purged again by N₂, sealed and heated at 100° C. for 16 h. Thereafter, the reaction was cooled to room temperature, saturated ammonium chloride solution was added, and the mixture was extracted with ethyl acetate. The combined organic extracts washed with brine, dried over sodium sulfate, filtered and concentrated. The resulting brown oil was purified utilizing silica gel chromatography (330 g×2 of silica, 0-15% gradient of EtOAc in hexanes) to afford 2-[2-(cyclohexylidenemethyl)-6-methyl-phenyl]-4,4,5,5-tetramethyl-1,3,2-dioxaborolane (24 g, 61%) as a yellow oil. ESI-MS m/z calc. 312.22607, found 313.4 (M+1)⁺; Retention time: 4.62 minutes; LC Method T.

### Step 6: tert-Butyl N-tert-butoxycarbonyl-N-[4-chloro-6-[2-(cyclohexylidenemethyl)-6-methyl-phenyl]-5-methyl-pyrimidin-2-yl]carbamate

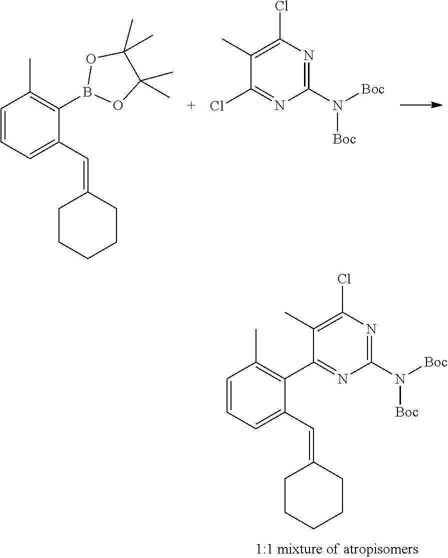 [SMILES: Cc1cccc(C=C2CCCCC2)c1B1OC(C)(C)C(C)(C)O1 ; Cc1c(Cl)nc(N(C(=O)OC(C)(C)C)C(=O)OC(C)(C)C)nc1Cl ; Cc1cccc(C=C2CCCCC2)c1-c1nc(N(C(=O)OC(C)(C)C)C(=O)OC(C)(C)C)nc(Cl)c1C]

To a slurry of tert-butyl N-tert-butoxycarbonyl-N-(4,6-dichloro-5-methyl-pyrimidin-2-yl)carbamate (12.4 g, 31.143 mmol), 2-[2-(cyclohexylidenemethyl)-6-methyl-phenyl]-4,4,5,5-tetramethyl-1,3,2-dioxaborolane (12 g, 36.509 mmol) and Cs₂CO₃ (25 g, 76.730 mmol) in dimethoxyethane (100 mL) and H₂O (20 mL) was added Pd(dppf)Cl₂ (1.1 g, 1.5033 mmol), and the mixture was vigorously stirred under nitrogen at 80° C. (reflux) for 3 h. The reaction mixture was cooled to ambient temperature and diluted with water (100 mL). The aqueous phase was separated and extracted with EtOAc (100 mL). The organic phase was washed with brine (150 mL), dried over anhydrous Na₂SO₄ and concentrated in vacuo. The crude product was purified by silica gel chromatography (330 g of silica, 0-20% gradient of EtOAc in hexane) to afford tert-butyl N-tert-butoxycarbonyl-N-[4-chloro-6-[2-(cyclohexylidenemethyl)-6-methyl-phenyl]-5-methyl-pyrimidin-2-yl]carbamate (8.64 g, 50%) as a colorless oil. ESI-MS m/z calc. 527.25507, found 528.6 (M+1)⁺; Retention time: 4.92 minutes; LC Method T.

### Step 7: 4-Chloro-6-[2-(cyclohexylidenemethyl)-6-methyl-phenyl]-5-methyl-pyrimidin-2-amine

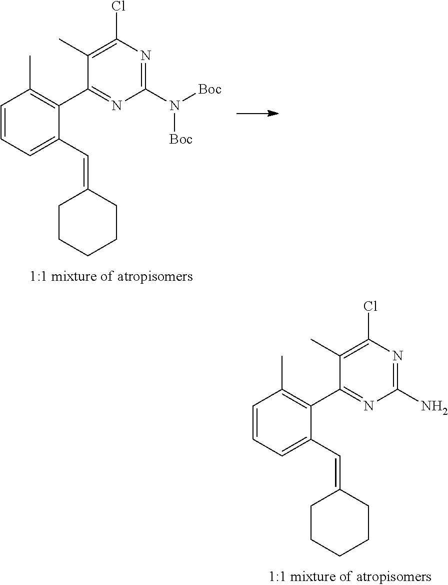 [SMILES: Cc1cccc(C=C2CCCCC2)c1-c1nc(N(C(=O)OC(C)(C)C)C(=O)OC(C)(C)C)nc(Cl)c1C ; Cc1cccc(C=C2CCCCC2)c1-c1nc(N)nc(Cl)c1C]

To a solution of tert-butyl N-tert-butoxycarbonyl-N-[4-chloro-6-[2-(cyclohexylidenemethyl)-6-methyl-phenyl]-5-methyl-pyrimidin-2-yl]carbamate (8.2 g, 14.751 mmol) in DCM (3 mL) was added a solution of HCl in dioxane (5.0 mL of 4 M, 20.0 mmol). The reaction mixture was stirred at ambient temperature for 5 h. Then, sat. NaHCO₃ solution was added. The organic phase was separated, dried and concentrated to afford 4-chloro-6-[2-(cyclohexylidenemethyl)-6-methyl-phenyl]-5-methyl-pyrimidin-2-amine (3.37 g, 69%); ESI-MS m/z calc. 327.15024, found 328.6 (M+1)⁺; Retention time: 3.72 minutes; LC Method T.

### Step 8: Methyl 3-[[4-chloro-6-[2-(cyclohexylidenemethyl)-6-methyl-phenyl]-5-methyl-pyrimidin-2-yl]sulfamoyl]benzoate

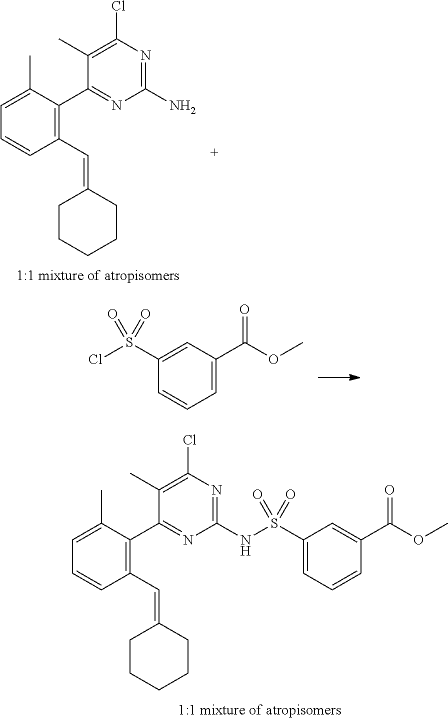

4-Chloro-6-[2-(cyclohexylidenemethyl)-6-methyl-phenyl]-5-methyl-pyrimidin-2-amine (2.6 g, 7.8511 mmol) was dissolved in THF (20 mL) and cooled in an ice bath under nitrogen. To the cold solution, methyl 3-chlorosulfonylbenzoate (2.8 g, 11.932 mmol) in solution in THF (10 mL) was added. At 0° C., lithium tert-pentoxide in heptane (5.5 g, 40% w/w, 23.384 mmol) was added dropwise and the reaction was stirred at room temperature for 2 h. The reaction was quenched with 1 N HCl solution (2 mL). The reaction was diluted with water (3 mL) and EtOAc (5 mL). The organic phase was separated, and the aqueous phase was extracted with EtOAc (5 mL). The combined organic phases were dried over sodium sulfate, filtered, and concentrated. The crude material was purified by silica gel chromatography (330 g silica, 0-40% gradient of acetone in hexane) to give methyl 3-[[4-chloro-6-[2-(cyclohexylidenemethyl)-6-methyl-phenyl]-5-methyl-pyrimidin-2-yl]sulfamoyl]benzoate (2.41 g, 57%) as a white solid. ESI-MS m/z calc. 525.1489, found 526.5 (M+1)⁺; Retention time: 4.24 minutes; LC Method T.

### Step 9: 3-[[4-Chloro-6-[2-(cyclohexylidenemethyl)-6-methyl-phenyl]-5-methyl-pyrimidin-2-yl]sulfamoyl]benzoic acid

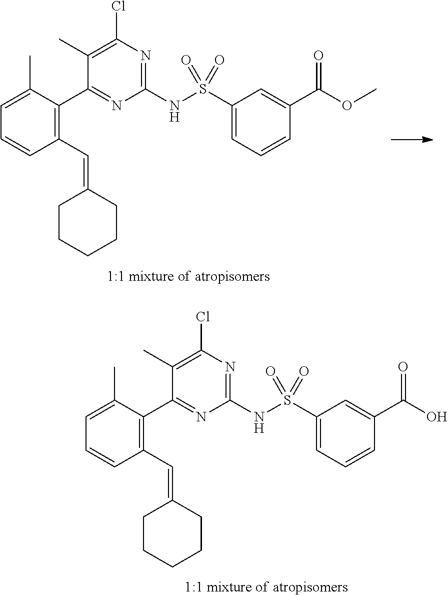 [SMILES: COC(=O)c1cccc(S(=O)(=O)Nc2nc(Cl)c(C)c(-c3c(C)cccc3C=C3CCCCC3)n2)c1 ; Cc1cccc(C=C2CCCCC2)c1-c1nc(NS(=O)(=O)c2cccc(C(=O)O)c2)nc(Cl)c1C]

To a solution of methyl 3-[[4-chloro-6-[2-(cyclohexylidenemethyl)-6-methyl-phenyl]-5-methyl-pyrimidin-2-yl]sulfamoyl]benzoate (1.5 g, 2.7659 mmol) in THF (10 mL) was added an aqueous solution of NaOH (2 mL of 3 M, 6.0 mmol) and stirred for 1 h at room temperature. The solution was acidified using 1 N HCl solution and extracted with ethyl acetate. The combined organic extracts were washed with brine. The organic layer was dried over sodium sulfate and concentrated in vacuo to give 3-[[4-chloro-6-[2-(cyclohexylidenemethyl)-6-methyl-phenyl]-5-methyl-pyrimidin-2-yl]sulfamoyl]benzoic acid (1.2 g, 82%) as a white solid. ESI-MS m/z calc. 511.13324, found 512.3 (M+1)⁺; Retention time: 3.8 minutes; LC Method T.

### Step 10: 3-[[4-[(2R)-2-(tert-Butoxycarbonylamino)-4,4-dimethyl-pentoxy]-6-[2-(cyclohexylidenemethyl)-6-methyl-phenyl]-5-methyl-pyrimidin-2-yl]sulfamoyl]benzoic acid

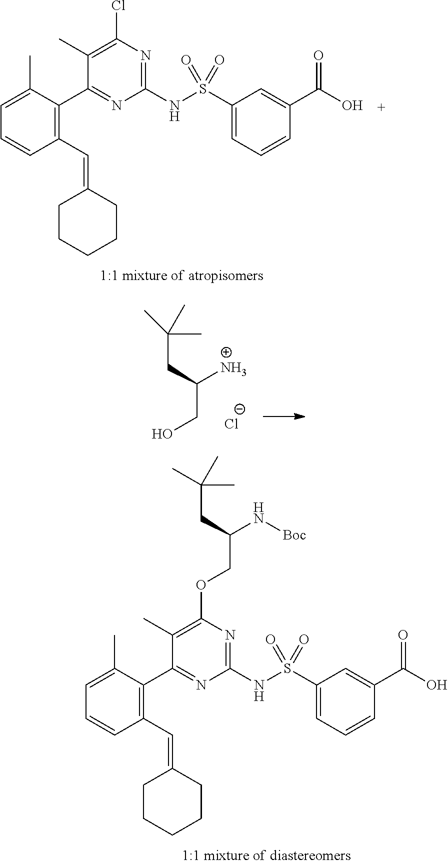

To a stirring solution of [(1R)-1-(hydroxymethyl)-3,3-dimethyl-butyl]ammonium chloride (1 g, 5.9638 mmol) in anhydrous THF (20 mL) at room temperature under nitrogen was added NaOtBu (1.5 g, 15.608 mmol). The reaction mixture was stirred for 10 min, and 3-[[4-chloro-6-[2-(cyclohexylidenemethyl)-6-methyl-phenyl]-5-methyl-pyrimidin-2-yl]sulfamoyl]benzoic acid (1.6 g, 3.0311 mmol) was added. The reaction mixture was stirred at room temperature for 2 h. Then, tert-butoxycarbonyl tert-butyl carbonate (4 g, 18.328 mmol) was added and the reaction mixture was stirred for 3 h. The reaction was quenched with saturated aqueous ammonium chloride (2 mL). The volatiles were removed in vacuo and the aqueous layer was acidified to pH ˜3 with 10% aqueous citric acid. The product was extracted with ethyl acetate, the combined organic layers were dried over anhydrous sodium sulfate and concentrated in vacuo. The residue was purified by silica gel chromatography (120 g silica, 0-40% gradient of acetone in hexane) to yield 3-[[4-[(2R)-2-(tert-butoxycarbonylamino)-4,4-dimethyl-pentoxy]-6-[2-(cyclohexylidenemethyl)-6-methyl-phenyl]-5-methyl-pyrimidin-2-yl]sulfamoyl]benzoic acid (2 g, 75%) as a colorless oil. ESI-MS m/z calc. 706.34, found 707.5 (M+1)⁺; Retention time: 4.13 minutes; LC Method T.

### Step 11: 3-[[4-[(2R)-2-(tert-Butoxycarbonylamino)-4,4-dimethyl-pentoxy]-6-[2-(cyclohexylmethyl)-6-methyl-phenyl]-5-methyl-pyrimidin-2-yl]sulfamoyl]benzoic acid

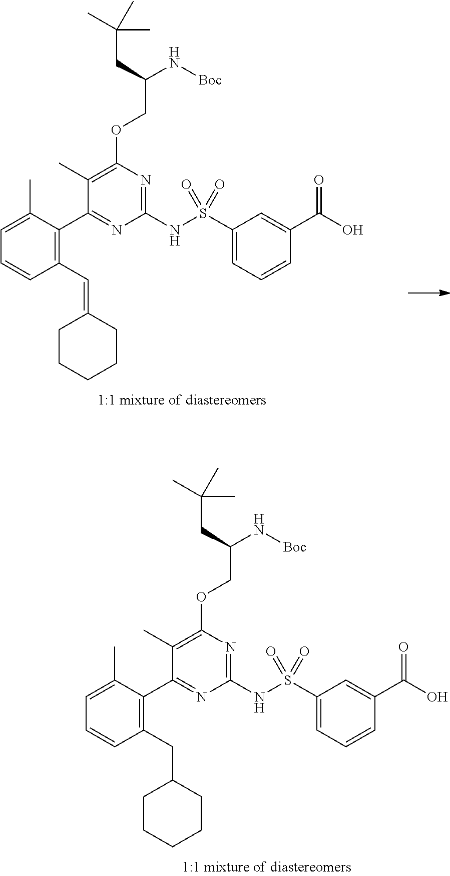 [SMILES: Cc1cccc(C=C2CCCCC2)c1-c1nc(NS(=O)(=O)c2cccc(C(=O)O)c2)nc(OC[C@@H](CC(C)(C)C)NC(=O)OC(C)(C)C)c1C ; Cc1cccc(CC2CCCCC2)c1-c1nc(NS(=O)(=O)c2cccc(C(=O)O)c2)nc(OC[C@@H](CC(C)(C)C)NC(=O)OC(C)(C)C)c1C]

3-[[4-[(2R)-2-(tert-Butoxycarbonylamino)-4,4-dimethyl-pentoxy]-6-[2-(cyclohexylidenemethyl)-6-methyl-phenyl]-5-methyl-pyrimidin-2-yl]sulfamoyl]benzoic acid (2 g, 2.2634 mmol) in MeOH (30 mL) was charged with Pd/C (25 mg). The reaction was stirred at rt for 12 h under a balloon of H₂. After completion, the reaction was filtered through Celite and concentrated in vacuo. The crude material was purified by reverse-phase HPLC (gradient 30-85% acetonitrile in water buffered by 5 mM HCl) to afford 3-[[4-[(2R)-2-(tert-butoxycarbonylamino)-4,4-dimethyl-pentoxy]-6-[2-(cyclohexylmethyl)-6-methyl-phenyl]-5-methyl-pyrimidin-2-yl]sulfamoyl]benzoic acid (1.25 g, 77%) as a white solid. ESI-MS m/z calc. 708.35565, found 709.6 (M+1)⁺; Retention time: 4.23 minutes; LC Method T.

### Step 12: 3-[[4-[(2R)-2-Amino-4,4-dimethyl-pentoxy]-6-[2-(cyclohexylmethyl)-6-methyl-phenyl]-5-methyl-pyrimidin-2-yl]sulfamoyl]benzoic acid

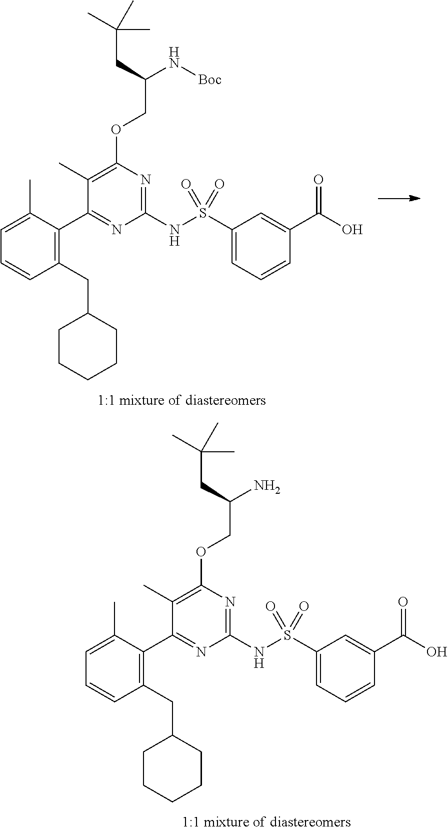 [SMILES: Cc1cccc(CC2CCCCC2)c1-c1nc(NS(=O)(=O)c2cccc(C(=O)O)c2)nc(OC[C@@H](CC(C)(C)C)NC(=O)OC(C)(C)C)c1C ; Cc1cccc(CC2CCCCC2)c1-c1nc(NS(=O)(=O)c2cccc(C(=O)O)c2)nc(OC[C@H](N)CC(C)(C)C)c1C]

To a solution of 3-[[4-[(2R)-2-(tert-butoxycarbonylamino)-4,4-dimethyl-pentoxy]-6-[2-(cyclohexylmethyl)-6-methyl-phenyl]-5-methyl-pyrimidin-2-yl]sulfamoyl]benzoic acid (1.25 g, 1.7456 mmol) in DCM (10 mL) was added a solution of HCl in dioxane (2 mL of 4 M, 8.0 mmol). The reaction mixture was stirred at room temperature for 5 h. After completion, the volatile material was evaporated in vacuo to afford 3-[[4-[(2R)-2-amino-4,4-dimethyl-pentoxy]-6-[2-(cyclohexylmethyl)-6-methyl-phenyl]-5-methyl-pyrimidin-2-yl]sulfamoyl]benzoic acid (hydrochloride salt) (1.1 g, 94%) as a white solid. ¹H NMR (500 MHz, DMSO-d₆) δ 8.42 (q, J=1.5, 1.5, 1.1 Hz, 1H), 8.29 (d, J=5.7 Hz, 3H), 8.15-8.04 (m, 2H), 7.66 (td, J=7.8, 2.7 Hz, 1H), 7.33-7.22 (m, 1H), 7.17-7.04 (m, 2H), 4.38-4.19 (m, 1H), 4.12 (apparent ddd, J=24.6, 12.0, 5.8 Hz, 2H), 2.19-2.02 (m, 1H), 2.07-1.94 (m, 1H), 1.90 (apparent d, J=22.8 Hz, 3H), 1.67 (d, J=4.0 Hz, 3H), 1.66-1.44 (m, 5H), 1.44-1.14 (m, 3H), 1.10-0.93 (m, 3H), 0.90 (d, J=16.2 Hz, 9H), 0.68 (q, J=12.0, 2H). ESI-MS m/z calc. 608.3032, found 609.5 (M+1)⁺; Retention time: 2.29 minutes; LC Method T.

### Step 13: (5M,11R)-6-[2-(Cyclohexylmethyl)-6-methylphenyl]-11-(2,2-dimethylpropyl)-7-methyl-12-{[5-(morpholin-4-yl)pyridin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (Compound 206) and (5P,11R)-6-[2-(cyclohexylmethyl)-6-methylphenyl]-11-(2,2-dimethylpropyl)-7-methyl-12-{[5-(morpholin-4-yl)pyridin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (Compound 207)

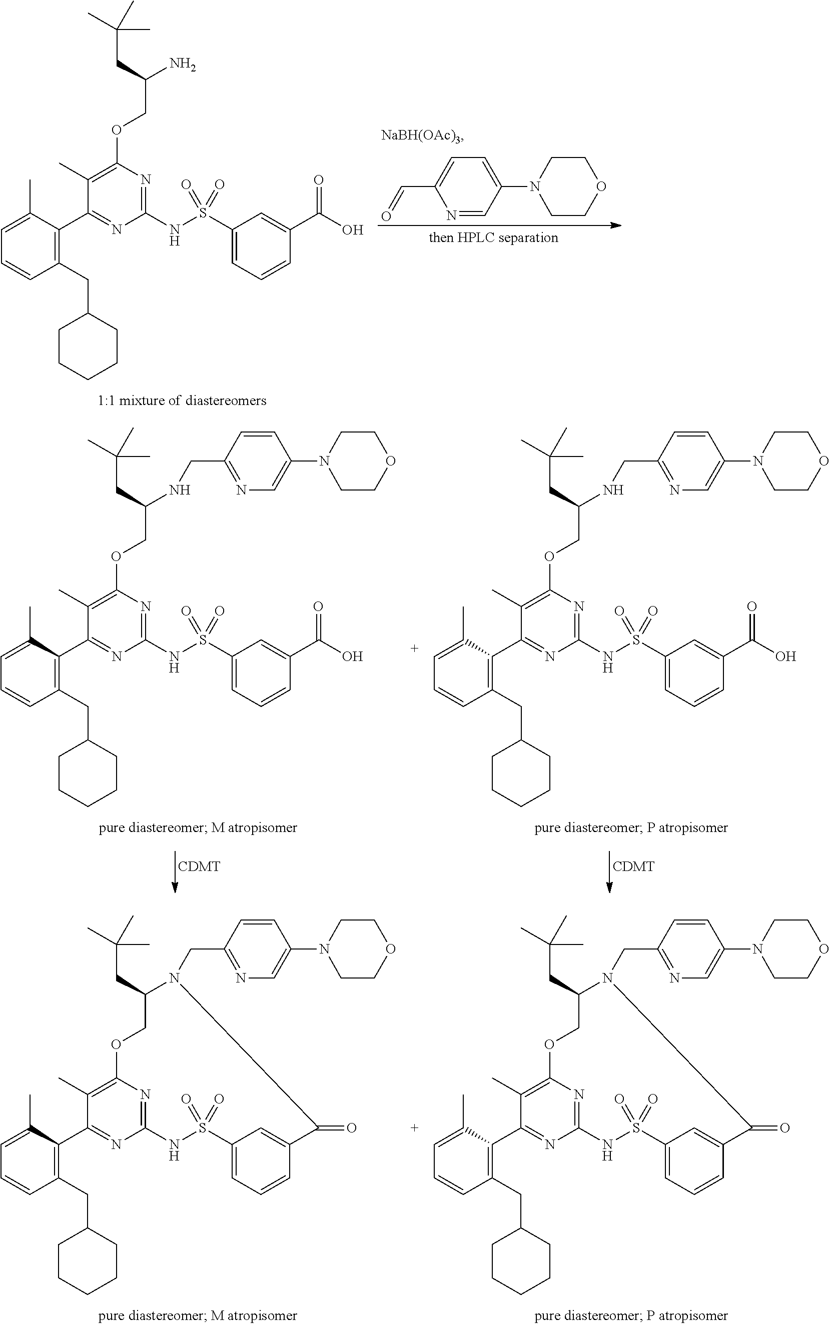

Stage 1: A 4-mL vial was charged with 3-[[4-[(2R)-2-amino-4,4-dimethyl-pentoxy]-6-[2-(cyclohexylmethyl)-6-methyl-phenyl]-5-methyl-pyrimidin-2-yl]sulfamoyl]benzoic acid;hydrochloride (211 mg, 0.3270 mmol), 5-morpholinopyridine-2-carbaldehyde (70 mg, 0.3642 mmol) and DCM (0.85 mL). The mixture was stirred at room temperature for 15 min. Then, sodium triacetoxyborohydride (98 mg, 0.4624 mmol) was added, the vial was purged with nitrogen, capped, and the mixture was stirred at room temperature for 30 min. More sodium triacetoxyborohydride (198 mg, 0.9342 mmol) was added, and the mixture was stirred at room temperature for 5 h. The reaction was quenched with a minimum amount of 1 N aqueous HCl. Methanol and DMSO were added. The solution was filtered and purified by reverse-phase preparative HPLC (C18 column) using a gradient (1-99%) of acetonitrile in aqueous 5 mM HCl to give two isomers. More polar (first to elute) isomer: 3-{[(3M)-4-[2-(cyclohexylmethyl)-6-methylphenyl]-6-{[(2R)-4,4-dimethyl-2-({[5-(morpholin-4-yl)pyridin-2-yl]methyl}amino)pentyl]oxy}-5-methylpyrimidin-2-yl]sulfamoyl}benzoic acid (hydrochloride salt) (73.6 mg, 55%). ESI-MS m/z calc. 784.3982, found 785.47 (M+1)⁺; Retention time: 1.71 minutes; LC Method A. Less polar (second to elute) isomer: 3-{[(3P)-4-[2-(cyclohexylmethyl)-6-methylphenyl]-6-{[(2R)-4,4-dimethyl-2-({[5-(morpholin-4-yl)pyridin-2-yl]methyl}amino)pentyl]oxy}-5-methylpyrimidin-2-yl]sulfamoyl}benzoic acid (hydrochloride salt) (82.5 mg, 61%). ESI-MS m/z calc. 784.3982, found 785.51 (M+1)⁺; Retention time: 1.74 minutes; LC Method A.

Stage 2: In two separate 20-mL vials, each isomer was combined under nitrogen with CDMT (53 mg, 0.3019 mmol) and anhydrous DMF (4 mL). The reaction was cooled in an ice-water bath, 4-methylmorpholine (0.11 mL, 1.001 mmol) was added and the mixture was stirred in the cooling bath that was allowed to warm to room temperature. After 19 h, the crude solution was diluted with DMSO, filtered and purified by reverse-phase preparative HPLC (C₁₈ column) using a gradient (1-99%) of acetonitrile in aqueous 5 mM HCl. After evaporation and trituration in EtOAc/hexanes, the products were isolated as white solids. Product originating from the more polar isomer: (5M, 11R)-6-[2-(cyclohexylmethyl)-6-methylphenyl]-11-(2,2-dimethylpropyl)-7-methyl-12-{[5-(morpholin-4-yl)pyridin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt) (39.4 mg, 30%). ¹H NMR (500 MHz, DMSO-d₆) δ 8.58 (s, 1H), 8.32 (d, J=3.0 Hz, 1H), 7.97-7.89 (m, 1H), 7.84-7.73 (m, 1H), 7.73-7.58 (m, 3H), 7.31 (t, J=7.6 Hz, 1H), 7.18 (d, J=7.6 Hz, 1H), 7.10 (d, J=7.7 Hz, 1H), 5.31 (dd, J=10.6, 4.4 Hz, 1H), 4.93 (d, J=15.2 Hz, 1H), 4.69 (d, J=15.7 Hz, 1H), 4.34 (q, J=10.0, 8.8 Hz, 1H), 4.03-3.94 (m, 1H), 3.76 (overlapped with water, t, J=4.8 Hz, 4H), 3.31-3.26 (overlapped with water, m, 4H), 2.04 (s, 3H), 1.98-1.83 (m, 3H), 1.61 (s, 3H), 1.58-1.44 (m, 3H), 1.44-1.33 (m, 2H), 1.29-1.24 (m, 1H), 1.17 (d, J=13.0 Hz, 1H), 1.09-0.90 (m, 3H), 0.65-0.43 (m, 11H). ESI-MS m/z calc. 766.38763, found 767.68 (M+1)⁺; Retention time: 1.83 minutes; LC Method A. Product originating from the less polar isomer: (5P,11R)-6-[2-(cyclohexylmethyl)-6-methylphenyl]-11-(2,2-dimethylpropyl)-7-methyl-12-{[5-(morpholin-4-yl)pyridin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (Hydrochloride salt) (34.8 mg, 26%). ¹H NMR (500 MHz, DMSO-d₆) δ 8.61 (s, 1H), 8.32 (d, J=3.0 Hz, 1H), 7.97 (d, J=6.6 Hz, 1H), 7.79 (s, 1H), 7.75-7.54 (m, 3H), 7.31 (t, J=7.6 Hz, 1H), 7.16 (d, J=7.7 Hz, 1H), 7.13 (d, J=7.6 Hz, 1H), 5.33 (dd, J=10.6, 4.5 Hz, 1H), 4.93 (d, J=15.5 Hz, 1H), 4.68 (d, J=15.6 Hz, 1H), 4.38 (t, J=11.3 Hz, 1H), 4.10-3.99 (m, 1H), 3.77 (overlapped with water, t, J=4.8 Hz, 4H), 3.32-3.24 (overlapped with water, m, 4H), 2.22 (qd, J=13.8, 7.0 Hz, 2H), 1.84 (dd, J=15.4, 9.1 Hz, 1H), 1.78 (s, 3H), 1.66-1.54 (m, 6H), 1.54-1.45 (m, 2H), 1.45-1.33 (m, 2H), 1.17-1.00 (m, 3H), 0.87-0.77 (m, 2H), 0.52 (s, 9H). ESI-MS m/z calc. 766.38763, found 767.65 (M+1)⁺; Retention time: 1.84 minutes; LC Method A.

### Example 71: Preparation of Compound 208 and Compound 209

### Step 1: 2-Bromo-1-isopropyl-3-methyl-benzene

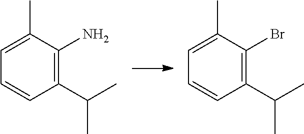

Into a solution of 2-isopropyl-6-methyl-aniline (10.048 g, 10.5 mL, 67.331 mmol) in concentrated HBr (100 mL) and water (100 mL) was added a solution of sodium nitrite (5.6 g, 81.165 mmol) in water (40 mL) at 0° C. After the addition, the reaction was stirred at 0° C. for 20 min. In a separate flask, a mixture of CuBr (9.67 g, 67.410 mmol) in concentrated HBr (100 mL) was heated to 60° C. The diazonium salt solution was added dropwise onto the CuBr mixture. After the addition was complete, the reaction was stirred at 60° C. for 1 h, and then it was cooled to rt. The solution was extracted with diethyl ether (3×100 mL). The combined ether layers were washed with sat. sodium bicarbonate (100 mL) and brine (75 mL), dried over anhydrous sodium sulfate and concentrated in vacuo. The residue was directly loaded onto a silica gel column and purified using 0 to 40% diethyl ether in hexane to furnish 2-bromo-1-isopropyl-3-methyl-benzene (7 g, 46%) as a clear oil. ¹H NMR (500 MHz, Chloroform-d) δ 7.18 (m, 1H), 7.15-7.11 (m, 1H), 7.11-7.06 (m, 1H), 3.59-3.42 (m, 1H), 2.44 (d, J=1.9 Hz, 3H), 1.26 (d, J=1.8 Hz, 3H), 1.25 (d, J=2.0 Hz, 3H).

### Step 2: (2-Isopropyl-6-methyl-phenyl)boronic acid

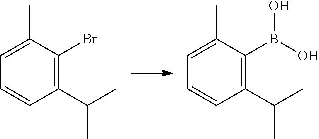

To a solution of 2-bromo-1-isopropyl-3-methyl-benzene (5.4 g, 25.339 mmol) in anhydrous THF (150 mL) at −78° C. was added dropwise a solution of n-BuLi in hexanes (12 mL of 2.5 M, 30.0 mmol). The solution was stirred at −78° C. for 15 min before adding trimethyl borate (7.9220 g, 8.5 mL, 76.237 mmol) dropwise. After the addition, the solution was allowed to warm to 0° C. and was stirred for 1 h. The solution was then quenched with 1 N HCl solution, and this was stirred for 3 h and then partitioned with EtOAc. The aqueous layer was extracted with EtOAc (2×20 mL). The organic layers were then combined, washed with brine, dried with anhydrous sodium sulfate, filtered, and concentrated under reduced pressure. The crude material was purified via silica gel column chromatography (eluting with 0-40% EtOAc in hexanes) to yield (2-isopropyl-6-methyl-phenyl)boronic acid (2.156 g, 45%) as a white solid. ¹H NMR (250 MHz, DMSO-d₆) δ 8.21-8.07 (m, 1H), 7.14 (t, J=7.5, 7.5 Hz, 1H), 7.03 (d, J=7.7 Hz, 1H), 6.92 (d, J=7.3 Hz, 1H), 2.82 (m, 1H), 2.26 (s, 3H), 1.39-0.96 (m, 6H).

### Step 3: tert-Butyl N-tert-butoxycarbonyl-N-[4-chloro-6-(2-isopropyl-6-methyl-phenyl)-5-methyl-pyrimidin-2-yl]carbamate

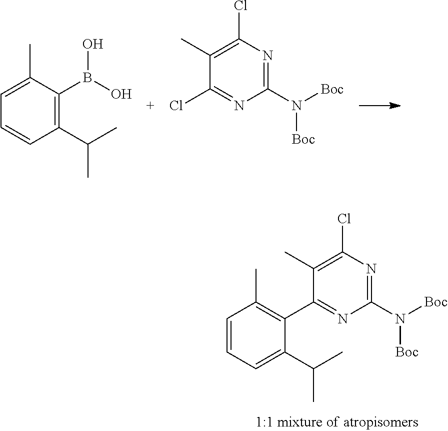 [SMILES: Cc1cccc(C(C)C)c1B(O)O ; Cc1c(Cl)nc(N(C(=O)OC(C)(C)C)C(=O)OC(C)(C)C)nc1Cl ; Cc1cccc(C(C)C)c1-c1nc(N(C(=O)OC(C)(C)C)C(=O)OC(C)(C)C)nc(Cl)c1C]

To a mixture of tert-butyl N-tert-butoxycarbonyl-N-(4,6-dichloro-5-methyl-pyrimidin-2-yl)carbamate (7.78 g, 20.568 mmol), (2-isopropyl-6-methyl-phenyl)boronic acid (3.2 g, 17.974 mmol), cesium carbonate (14.5 g, 44.503 mmol), and Pd(dppf)Cl₂ (1.47 g, 1.8001 mmol) was added a degassed solvent mixture of dimethoxyethane (70 mL) and water (70 mL). During the addition of the solvents, the reaction vial was continuously flushed with nitrogen. Once the addition was finished, the vial was sealed and heated to 80° C., and the reaction mixture was stirred for 2 h. The mixture was then quenched with water (40 mL) and EtOAc (70 mL). The aqueous layer was extracted with EtOAc (2×50 mL). The organic layers were combined, washed with brine, dried with anhydrous sodium sulfate, filtered, and concentrated under reduced pressure. The crude was purified via silica gel column chromatography (eluting with a gradient of 0 to 30% EtOAc in hexanes) to yield tert-butyl N-tert-butoxycarbonyl-N-[4-chloro-6-(2-isopropyl-6-methyl-phenyl)-5-methyl-pyrimidin-2-yl]carbamate (5.36 g, 41%) as a white solid. ESI-MS m/z calc. 475.2238, found 476.2 (M+1)⁺; Retention time: 4.14 minutes; LC Method T.

### Step 4: 4-Chloro-6-(2-isopropyl-6-methyl-phenyl)-5-methyl-pyrimidin-2-amine

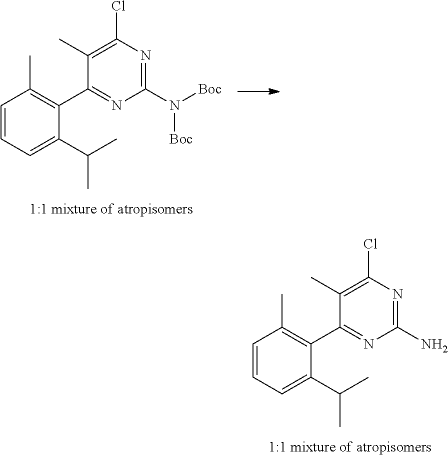 [SMILES: Cc1cccc(C(C)C)c1-c1nc(N(C(=O)OC(C)(C)C)C(=O)OC(C)(C)C)nc(Cl)c1C ; Cc1cccc(C(C)C)c1-c1nc(N)nc(Cl)c1C]

To a solution of tert-butyl N-tert-butoxycarbonyl-N-[4-chloro-6-(2-isopropyl-6-methyl-phenyl)-5-methyl-pyrimidin-2-yl]carbamate (5.36 g, 7.3192 mmol) in anhydrous DCM (30 mL) at 0° C. was added a solution of HCl in dioxane (30 mL of 4 M, 120.0 mmol). The solution was then warmed to RT and stirred for 3 h. The solution was then quenched with NaHCO₃(75 mL) and partitioned with DCM (50 mL). The aqueous layer was extracted with DCM (2×50 mL). The organic layers were then combined, washed with brine, dried with anhydrous sodium sulfate, filtered, and concentrated under reduced pressure. The crude product was purified via silica gel column chromatography (eluting with a gradient of 0 to 40% ether in hexanes) to yield 4-chloro-6-(2-isopropyl-6-methyl-phenyl)-5-methyl-pyrimidin-2-amine (2.5 g, >100% yield) as a white solid. ¹H NMR (500 MHz, DMSO-d₆) δ 7.33-7.18 (m, 2H), 7.10 (d, J=7.3 Hz, 1H), 6.91 (s, 2H), 1.93 (s, 3H), 1.77 (s, 3H), 1.07 (apparent dd, J=16.5, 6.8 Hz, 6H). ESI-MS m/z calc. 275.11893, found 276.3 (M+1)⁺; Retention time: 2.5 minutes; LC Method W.

### Step 5: Methyl 3-[[4-chloro-6-(2-isopropyl-6-methyl-phenyl)-5-methyl-pyrimidin-2-yl]sulfamoyl]benzoate

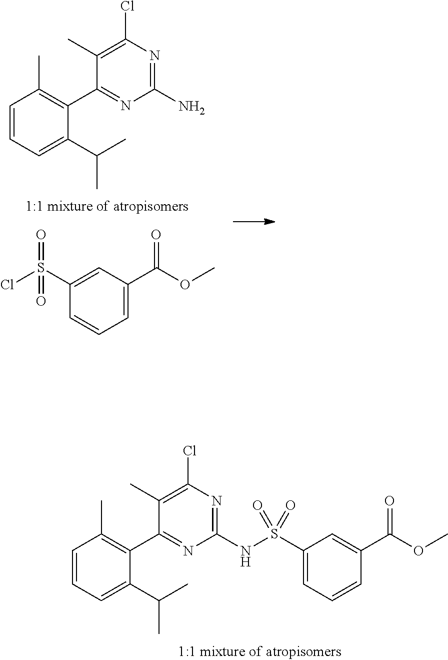 [SMILES: Cc1cccc(C(C)C)c1-c1nc(N)nc(Cl)c1C ; COC(=O)c1cccc(S(=O)(=O)Cl)c1 ; COC(=O)c1cccc(S(=O)(=O)Nc2nc(Cl)c(C)c(-c3c(C)cccc3C(C)C)n2)c1]

To a solution of 4-chloro-6-(2-isopropyl-6-methyl-phenyl)-5-methyl-pyrimidin-2-amine (2.5 g, 8.3401 mmol) and methyl 3-chlorosulfonylbenzoate (2.57 g, 10.952 mmol) in anhydrous THF (25 mL) at 0° C. was added dropwise a solution of lithium tert-amoxide in heptanes (5.2560 g, 18 mL of 40% w/w, 22.347 mmol), which was then stirred at 0° C. for 5 min after addition. The solution was then warmed to RT and stirred for 2 h. The reaction was then quenched with 1 N HCl solution (50 mL) and partitioned with EtOAc (50 mL). The aqueous layer was extracted with EtOAc (2×50 mL). The organic layers were then combined, washed with brine, dried with anhydrous sodium sulfate, filtered, and concentrated under reduced pressure to yield methyl 3-[[4-chloro-6-(2-isopropyl-6-methyl-phenyl)-5-methyl-pyrimidin-2-yl]sulfamoyl]benzoate (4.75 g, 65%) as a yellow solid. ESI-MS m/z calc. 473.1176, found 474.1 (M+1)⁺; Retention time: 3.57 minutes; LC Method T.

### Step 6: 3-[[4-Chloro-6-(2-isopropyl-6-methyl-phenyl)-5-methyl-pyrimidin-2-yl]sulfamoyl]benzoic acid

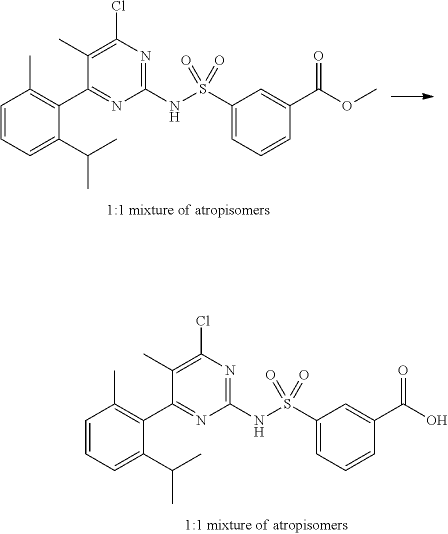 [SMILES: COC(=O)c1cccc(S(=O)(=O)Nc2nc(Cl)c(C)c(-c3c(C)cccc3C(C)C)n2)c1 ; Cc1cccc(C(C)C)c1-c1nc(NS(=O)(=O)c2cccc(C(=O)O)c2)nc(Cl)c1C]

To a solution of methyl 3-[[4-chloro-6-(2-isopropyl-6-methyl-phenyl)-5-methyl-pyrimidin-2-yl]sulfamoyl]benzoate (4.75 g, 5.4117 mmol) in THF (17 mL) was added aqueous NaOH (30 mL of 1 M, 30.0 mmol). The reaction was stirred at RT for 4 h. The reaction was then quenched with 1 N HCl solution (50 mL), and partitioned with EtOAc (30 mL). The aqueous layer was then extracted with EtOAc (2×20 mL). The organic layers were then combined, washed with brine, dried with anhydrous sodium sulfate, filtered, and concentrated under reduced pressure. The crude was purified via reverse-phase HPLC (gradient 60-100% acetonitrile in water buffered by 0.1% TFA) and then by silica gel column chromatography (eluting with a gradient of 0-70% acetone in hexanes buffered by 0.1% AcOH) to yield 3-[[4-chloro-6-(2-isopropyl-6-methyl-phenyl)-5-methyl-pyrimidin-2-yl]sulfamoyl]benzoic acid (2.28 g, 73%) as a white solid. ¹H NMR (500 MHz, DMSO-d₆) δ 13.39 (s, 1H), 12.24 (s, 1H), 8.39 (d, J=1.8 Hz, 1H), 8.13 (m, 1H), 8.06 (m, 1H), 7.62 (t, J=7.8, 7.8 Hz, 1H), 7.31 (t, J=7.7, 7.7 Hz, 1H), 7.22 (d, J=7.8 Hz, 1H), 7.09 (d, J=7.4 Hz, 1H), 2.17 (m, 1H), 1.83 (s, 3H), 1.64 (s, 3H), 1.04 (d, J=6.8 Hz, 3H), 0.86 (d, J=6.8 Hz, 3H). ESI-MS m/z calc. 459.10196, found 460.3 (M+1)⁺; Retention time: 2.69 minutes; LC Method W.

### Step 7: 3-[[4-[(2R)-2-amino-4,4-dimethyl-pentoxy]-6-(2-isopropyl-6-methyl-phenyl)-5-methyl-pyrimidin-2-yl]sulfamoyl]benzoic acid

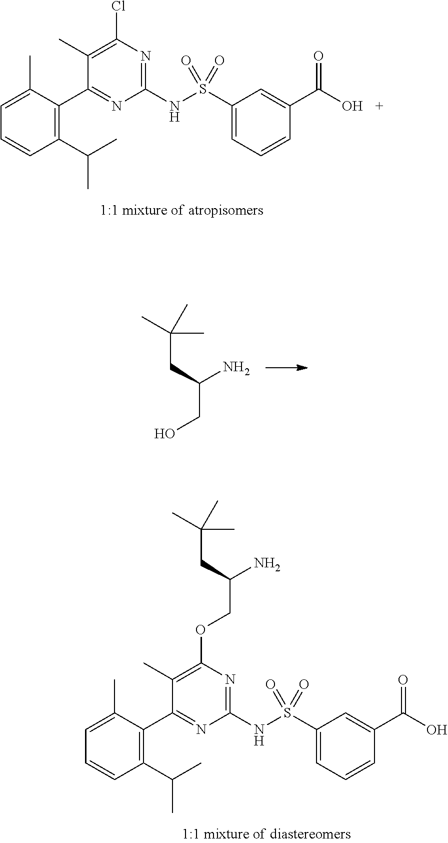

To a solution of 3-[[4-chloro-6-(2-isopropyl-6-methyl-phenyl)-5-methyl-pyrimidin-2-yl]sulfamoyl]benzoic acid (1.07 g, 2.2380 mmol) and (2R)-2-amino-4,4-dimethyl-pentan-1-ol (282 mg, 2.1491 mmol) in anhydrous THF (15 mL) was added sodium tert-butoxide (895 mg, 9.3129 mmol). The reaction was stirred at RT for 2 h. The reaction was then quenched with 1 N HCl solution (35 mL) and partitioned with EtOAc (35 mL). The aqueous layer was then extracted with EtOAc (2×30 mL). The organic layers were combined, washed with brine, dried with anhydrous sodium sulfate, filtered, and concentrated under reduced pressure. The crude product was then purified via reverse-phase column chromatography (gradient 30-70% acetonitrile in water buffered by 0.1% TFA). The pure fractions were concentrated until some water was remaining, and the solution was mixed with 1 N HCl solution (80 mL) to displace the TFA to make the HCl salt. The solution was dried via lyophilizer to yield 3-[[4-[(2R)-2-amino-4,4-dimethyl-pentoxy]-6-(2-isopropyl-6-methyl-phenyl)-5-methyl-pyrimidin-2-yl]sulfamoyl]benzoic acid (hydrochloride salt) (827.5 mg, 60%) as a white solid. ¹H NMR (500 MHz, DMSO-d₆) δ 8.43 (m, 1H), 8.21-8.05 (m, 4H), 7.66 (t, J=7.8, 7.8 Hz, 1H), 7.34 (t, J=7.7, 7.7 Hz, 1H), 7.26 (d, J=7.9 Hz, 1H), 7.14 (d, J=7.5 Hz, 1H), 4.36-4.27 (m, 1H), 4.24 (d, J=11.9 Hz, 1H), 3.57 (s, 1H), 2.40-2.31 (m, 1H), 1.88 (s, 3H), 1.67 (s, 3H), 1.62 (m, 1H), 1.52 (m, 1H), 1.05 (m, 6H), 0.93 (d, J=1.3 Hz, 9H). ESI-MS m/z calc. 554.2563, found 555.4 (M+1)⁺; Retention time: 2.03 minutes; LC Method W.

### Step 8: (5M,11R)-11-(2,2-dimethylpropyl)-7-methyl-6-[2-methyl-6-(propan-2-yl)phenyl]-12-{[5-(morpholin-4-yl)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (Compound 208) and (5P,11R)-11-(2,2-dimethylpropyl)-7-methyl-6-[2-methyl-6-(propan-2-yl)phenyl]-12-{[5-(morpholin-4-yl)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (Compound 209)

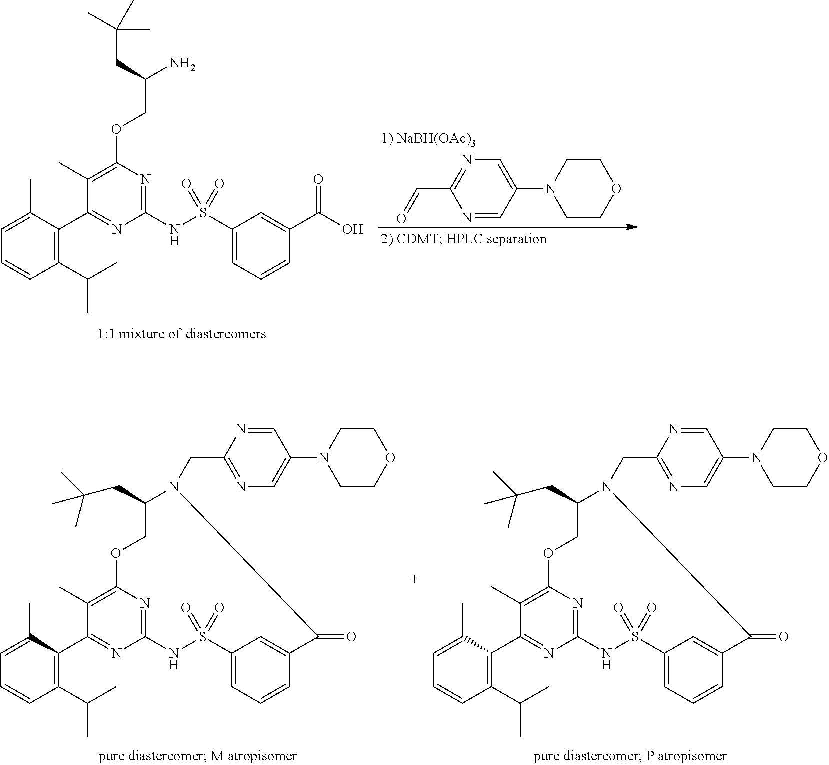

Stage 1: A 4-mL vial was charged with 3-[[4-[(2R)-2-amino-4,4-dimethyl-pentoxy]-6-(2-isopropyl-6-methyl-phenyl)-5-methyl-pyrimidin-2-yl]sulfamoyl]benzoic acid;hydrochloride (100 mg, 0.1692 mmol), 5-morpholinopyrimidine-2-carbaldehyde hydrochloride (46 mg, 0.2003 mmol) and DCM (400 μL). The mixture was stirred at room temperature for 30 min. Sodium triacetoxyboranuide (47 mg, 0.2218 mmol) was added, the vial was purged with nitrogen, capped and the mixture was stirred at room temperature for 1 h. More sodium triacetoxyboranuide (111 mg, 0.5237 mmol) was added and the mixture was stirred at room temperature for 3.5 h. The reaction was quenched with 1 N aqueous HCl. Methanol and DMSO were added. The solution was filtered and purified by reverse-phase preparative HPLC (C₁₈ column) using a gradient (1-99%) of acetonitrile in aqueous 5 mM HCl to give 3-[[4-[(2R)-4,4-dimethyl-2-[(5-morpholinopyrimidin-2-yl)methylamino]pentoxy]-6-(2-isopropyl-6-methyl-phenyl)-5-methyl-pyrimidin-2-yl]sulfamoyl]benzoic acid (hydrochloride salt) (81 mg, 62%) as a solid. ESI-MS m/z calc. 731.3465, found 732.407 (M+1)⁺; Retention time: 2.00 minutes, second isomer retention time: 2.01 minutes (ratio 1:1); LC Method I.

Stage 2: The material from Stage 1 was combined under nitrogen with CDMT (28 mg, 0.1595 mmol) and anhydrous DMF (1.9 mL). The reaction was cooled in an ice-water bath, 4-methylmorpholine (58 μL, 0.5275 mmol) was added and the mixture was stirred in the cooling bath, allowing to warm to room temperature over 19 h. Thereafter, the crude solution was diluted with DMSO, filtered and purified by reverse-phase preparative HPLC (C₁₈ column) using a gradient (1-99%) of acetonitrile in aqueous 5 mM HCl to provide two separate isomers as solids. First to elute, more polar isomer: (5M,11R)-11-(2,2-dimethylpropyl)-7-methyl-6-[2-methyl-6-(propan-2-yl)phenyl]-12-{[5-(morpholin-4-yl)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (7.3 mg, 11%). ¹H NMR (400 MHz, DMSO-d₆) δ 13.41-11.24 (broad m, 1H), 8.71 (s, 1H), 8.50 (s, 2H), 7.95 (d, J=7.3 Hz, 1H), 7.67 (br s, 2H), 7.36 (t, J=7.8 Hz, 1H), 7.25 (d, J=7.8 Hz, 1H), 7.18 (d, J=7.5 Hz, 1H), 5.41 (dd, J=10.9, 4.3 Hz, 1H), 4.85 (d, J=16.4 Hz, 1H), 4.62 (d, J=16.5 Hz, 1H), 4.24 (t, J=11.1 Hz, 1H), 4.16-4.01 (m, 1H), 3.75 (dd, J=6.1, 3.7 Hz, 4H), 3.23 (dd, J=5.8, 3.9 Hz, 4H), 2.09 (p, J=6.6 Hz, 1H), 2.04 (s, 3H), 1.82 (dd, J=15.2, 8.6 Hz, 1H), 1.60 (s, 3H), 1.35 (d, J=15.1 Hz, 1H), 1.05-0.89 (m, 6H), 0.53 (s, 9H). ESI-MS m/z calc. 713.33594, found 714.47 (M+1)⁺: Retention time: 1.89 minutes; LC Method A. Second to elute, less polar isomer: (5P,11R)-11-(2,2-dimethylpropyl)-7-methyl-6-[2-methyl-6-(propan-2-yl)phenyl]-12-{[5-(morpholin-4-yl)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (11.1 mg, 18%). ¹H NMR (400 MHz, DMSO-d₆) δ 13.35-11.53 (broad m, 1H), 8.71 (s, 1H), 8.50 (s, 2H), 7.94 (br s, 1H), 7.66 (br s, 2H), 7.36 (br s, 1H), 7.30 (br d, J=7.7 Hz, 1H), 7.13 (br s, 1H), 5.40 (r s, 1H), 4.85 (d, J=16.4 Hz, 1H), 4.62 (d, J=16.5 Hz, 1H), 4.21 (t, J=11.2 Hz, 1H), 4.10-3.96 (m, 1H), 3.75 (dd, J=6.0, 3.7 Hz, 4H), 3.26-3.16 (m, 4H), 2.53 (overlapped with DMSO, m, 1H), 1.93-1.66 (m, 4H), 1.60 (br s, 3H), 1.37 (d, J=14.9 Hz, 1H), 1.15 (d, J=6.2 Hz, 3H), 1.09 (d, J=6.8 Hz, 3H), 0.55 (s, 9H). ESI-MS m/z calc. 713.33594, found 714.4 (M+1)⁺; Retention time: 1.92 minutes; LC Method A.

### Example 72: Preparation of Compound 210 and Compound 211

### Step 1: 1-Methyl-3-(2-methylprop-1-enyl)-2-nitro-benzene

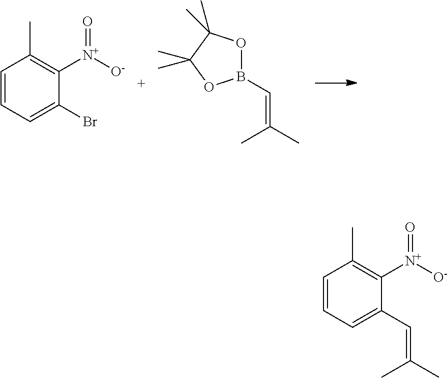

To a stirring solution of 1-bromo-3-methyl-2-nitro-benzene (27.78 g, 128.59 mmol) and 4,4,5,5-tetramethyl-2-(2-methylprop-1-enyl)-1,3,2-dioxaborolane (24.75 g, 135.94 mmol) in 1,4-dioxane (650 mL) at room temperature was added water (200 mL). The reaction mixture was degassed with nitrogen for 30 min. Under nitrogen, sodium carbonate (40.986 g, 386.70 mmol) and [1,1′-bis(diphenylphosphino)ferrocene]dichloropalladium(II) (9.082 g, 12.412 mmol) were added. The reaction mixture was heated to reflux (110° C.) under nitrogen for 18 h. Then, after cooling to room temperature, water (200 mL) and ethyl acetate (400 mL) were added, and the reaction mixture was vigorously stirred for 10 min. Two layers were separated, and the aqueous layer was extracted with ethyl acetate (2×300 mL). The combined organic layers were washed with brine (150 mL), dried over anhydrous sodium sulfate and concentrated. The crude was purified by silica gel chromatography using 0-5% ethyl acetate gradient in hexanes to afford 1-methyl-3-(2-methylprop-1-enyl)-2-nitro-benzene (20.2 g, 76%) as a yellow oil. ¹H NMR (500 MHz, DMSO-d₆) δ 7.46 (t, J=7.7 Hz, 1H), 7.34 (d, J=7.7 Hz, 1H), 7.23 (d, J=7.6 Hz, 1H), 6.06 (s, 1H), 2.25 (s, 3H), 1.85 (d, J=1.4 Hz, 3H), 1.68 (d, J=1.4 Hz, 3H). ESI-MS m/z calc. 191.09464, Retention time: 6.12 minutes; LC Method S.

### Step 2: 2-Isobutyl-6-methyl-aniline

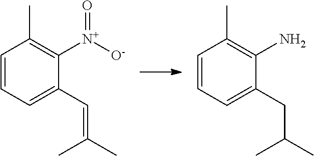

To a stirring solution of 1-methyl-3-(2-methylprop-1-enyl)-2-nitro-benzene (20.2 g, 100.35 mmol) in methanol (300 mL) at room temperature under ambient conditions was added palladium on carbon (10.28 g, 10% w/w, 9.6598 mmol). The head space was purged with nitrogen, followed by hydrogen. The reaction mixture was stirred under the atmosphere of hydrogen (1 atm) for 30 h. The reaction mixture was filtered through a pad of Celite, and the filter cake was washed with methanol (3×50 mL). The combined filtrate was concentrated under vacuum. The crude was purified by silica gel chromatography using 0-5% ethyl acetate gradient in hexanes to afford 2-isobutyl-6-methyl-aniline (16.06 g, 93%) as an amber oil. ¹H NMR (500 MHz, DMSO-d₆) δ 6.79 (d, J=7.4 Hz, 1H), 6.72 (d, J=7.4 Hz, 1H), 6.42 (t, J=7.4 Hz, 1H), 4.41 (s, 2H), 2.33 (d, J=7.3 Hz, 2H), 2.06 (s, 3H), 1.91-1.80 (m, 1H), 0.87 (d, J=6.6 Hz, 6H). ESI-MS m/z calc. 163.1361, found 164.4 (M+1)⁺; Retention time: 2.96 minutes; LC Method S.

### Step 3: 2-Bromo-1-isobutyl-3-methyl-benzene

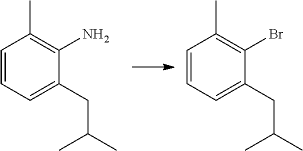

To a stirring suspension of 2-isobutyl-6-methyl-aniline (16.428 g, 100.63 mmol) in a mixture of concentrated (48%) HBr (140 mL) and water (140 mL) at 0° C. was added dropwise a solution of sodium nitrite (8.331 g, 120.75 mmol) in water (60 mL). After the addition was complete, the reaction mixture was stirred at 0° C. for 45 min. To the reaction mixture was slowly added a solution of copper(I) bromide (17.332 g, 120.82 mmol) in concentrated (48%) HBr (140 mL). After the addition was complete, the reaction mixture was heated to 60° C. for 1 h. After cooling to room temperature, diethyl ether (300 mL) was added and the reaction mixture was stirred for 15 min. The layers were separated, and the aqueous layer was extracted with diethyl ether (2×150 mL). The combined organic layers were washed with brine (100 mL), dried over anhydrous sodium sulfate and concentrated. The crude was purified by silica gel chromatography using hexanes to afford 2-bromo-1-isobutyl-3-methyl-benzene (11.77 g, 44%) as a colorless oil. ¹H NMR (500 MHz, DMSO-d) δ 7.22-7.15 (m, 2H), 7.10-7.06 (m, 1H), 2.60 (d, J=7.2 Hz, 2H), 2.36 (s, 3H), 1.98-1.87 (m, 1H), 0.89 (d, J=6.6 Hz, 6H).

### Step 4: (2-Isobutyl-6-methyl-phenyl)boronic acid

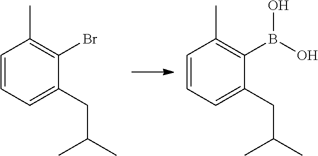

To a stirring solution of 2-bromo-1-isobutyl-3-methyl-benzene (11.77 g, 46.636 mmol) in anhydrous THF (200 mL) at −78° C. under nitrogen was added dropwise a solution of n-butyllithium (23 mL of 2.5 M, 57.500 mmol) in hexanes. After the addition was complete, the reaction mixture was stirred at −78° C. for 1 h. To the reaction mixture was added dropwise trimethyl borate (19.530 g, 21 mL, 187.95 mmol). After the addition was complete, the reaction mixture was stirred at −78° C. for 15 min, then allowed to warm to 0° C. and stirred for 1 h. To the reaction mixture was slowly added aqueous HCl (150 mL of 1 M, 150.0 mmol). After the addition was complete, the reaction mixture was allowed to warm to room temperature and stirred for 3 h. Ethyl acetate (200 mL) was added, and the reaction mixture was vigorously stirred for 10 min. The layers were separated, and the aqueous layer was extracted with ethyl acetate (2×120 mL). The combined organic layers were washed with brine (100 mL), dried over anhydrous sodium sulfate and concentrated. The crude was purified by silica gel chromatography using a 0-20% gradient of acetone in hexanes. The product was repurified by silica gel chromatography using a gradient of 0-40% diethyl ether in hexanes to afford (2-isobutyl-6-methyl-phenyl)boronic acid (3.2 g, 32%) as a white solid. ¹H NMR (500 MHz, DMSO-d₆) δ 8.06 (s, 2H), 7.08 (t, J=7.5 Hz, 1H), 6.92 (d, J=7.6 Hz, 1H), 6.87 (d, J=7.6 Hz, 1H), 2.43 (d, J=7.2 Hz, 2H), 2.27 (s, 3H), 1.92-1.81 (m, 1H), 0.85 (d, J=6.6 Hz, 6H). ESI-MS m/z calc. 192.13216, Retention time: 3.98 minutes; LC Method S.

### Step 5: tert-Butyl N-tert-butoxycarbonyl-N-[4-chloro-6-(2-isobutyl-6-methyl-phenyl)-5-methyl-pyrimidin-2-yl]carbamate and tert-butyl N-[4-chloro-6-(2-isobutyl-6-methyl-phenyl)-5-methyl-pyrimidin-2-yl]carbamate

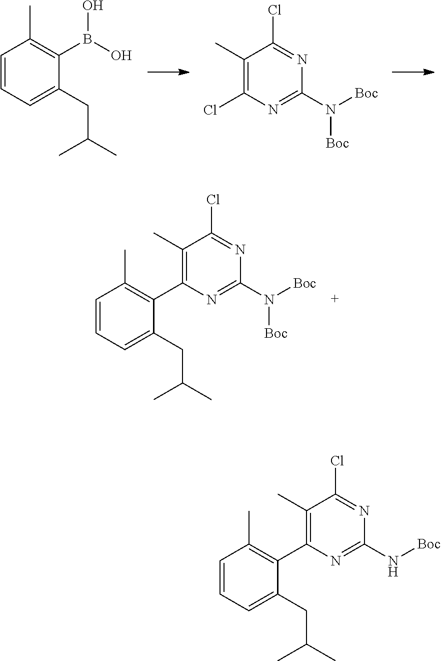 [SMILES: Cc1cccc(CC(C)C)c1B(O)O ; Cc1c(Cl)nc(N(C(=O)OC(C)(C)C)C(=O)OC(C)(C)C)nc1Cl ; Cc1cccc(CC(C)C)c1-c1nc(N(C(=O)OC(C)(C)C)C(=O)OC(C)(C)C)nc(Cl)c1C ; Cc1cccc(CC(C)C)c1-c1nc(NC(=O)OC(C)(C)C)nc(Cl)c1C]

To a stirring solution of tert-butyl N-tert-butoxycarbonyl-N-(4,6-dichloro-5-methyl-pyrimidin-2-yl)carbamate (6.235 g, 16.484 mmol) and (2-isobutyl-6-methyl-phenyl)boronic acid (3.2 g, 14.995 mmol) in 1,2-dimethoxyethane (81 mL) at room temperature was added water (27 mL). The reaction mixture was degassed with nitrogen for 20 min. Under nitrogen, cesium carbonate (14.644 g, 44.945 mmol) and [1,1′-bis(diphenylphosphino)ferrocene]dichloropalladium(II) (1.101 g, 1.5047 mmol) were added. The reaction mixture was heated at 95° C. for 3 h. After cooling to room temperature, water (100 mL) and ethyl acetate (100 mL) were added, and the reaction mixture was vigorously stirred for 5 min. Two layers were separated, and the aqueous layer was extracted with ethyl acetate (2×80 mL). The combined organic layers were washed with brine (60 mL), dried over anhydrous sodium sulfate and concentrated. The crude product was purified by silica gel chromatography using 0-15% diethyl ether gradient in hexanes, then by reverse-phase HPLC using a gradient of 85-100% acetonitrile in water (0.15% THF buffer). The pure product fractions were combined and basified with saturated aqueous NaHCO₃ to pH ˜8. The volatiles were removed in vacuo, and the residual aqueous layer was extracted with ethyl acetate (3×80 mL). The combined organic layers were washed with brine (40 mL), dried over anhydrous sodium sulfate and concentrated to afford a mixture of: tert-butyl N-tert-butoxycarbonyl-N-[4-chloro-6-(2-isobutyl-6-methyl-phenyl)-5-methyl-pyrimidin-2-yl]carbamate (3.1 g, 40%); ESI-MS m/z calc. 489.23944, found 490.3 (M+1)⁺; Retention time: 8.39 minutes; LC Method S, as well as tert-butyl N-[4-chloro-6-(2-isobutyl-6-methyl-phenyl)-5-methyl-pyrimidin-2-yl]carbamate (0.897 g, 15%); ESI-MS m/z calc. 389.187, found 390.6 (M+1)⁺; Retention time: 7.49 minutes; LC Method S.

### Step 6: 4-Chloro-6-(2-isobutyl-6-methyl-phenyl)-5-methyl-pyrimidin-2-amine

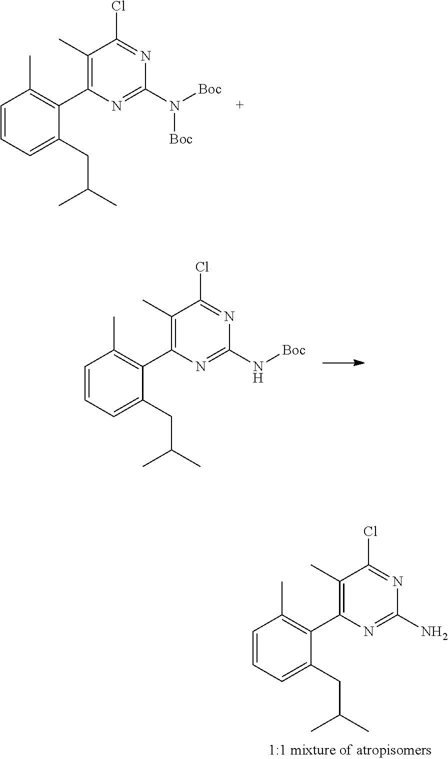 [SMILES: Cc1cccc(CC(C)C)c1-c1nc(N(C(=O)OC(C)(C)C)C(=O)OC(C)(C)C)nc(Cl)c1C ; Cc1cccc(CC(C)C)c1-c1nc(NC(=O)OC(C)(C)C)nc(Cl)c1C ; Cc1cccc(CC(C)C)c1-c1nc(N)nc(Cl)c1C]

To a mixture of tert-butyl N-tert-butoxycarbonyl-N-[4-chloro-6-(2-isobutyl-6-methyl-phenyl)-5-methyl-pyrimidin-2-yl]carbamate and tert-butyl N-[4-chloro-6-(2-isobutyl-6-methyl-phenyl)-5-methyl-pyrimidin-2-yl]carbamate (3.997 g, 8.1566 mmol) in DCM (50 mL) at room temperature was slowly added a solution of HCl (13 mL of 4 M, 52.0 mmol) in dioxane. After the addition was complete, the reaction mixture was sealed and stirred for 18 h. The volatiles were removed in vacuo, then the obtained residue was resuspended in saturated aqueous NaHCO₃(150 mL) and stirred vigorously for 10 min. Ethyl acetate (120 mL) was added, and the mixture was vigorously stirred for 10 min (until all the solid was dissolved). The layers were separated, and the aqueous layer was extracted with ethyl acetate (2×100 mL). The combined organic layers were washed with brine (60 mL), dried over anhydrous sodium sulfate and concentrated to afford 4-chloro-6-(2-isobutyl-6-methyl-phenyl)-5-methyl-pyrimidin-2-amine (2.349 g, 94%) as a white solid. ESI-MS m/z calc. 289.13458, found 290.3 (M+1)⁺; Retention time: 5.67 minutes; LC Method S.

### Step 7: Methyl 3-[[4-chloro-6-(2-isobutyl-6-methyl-phenyl)-5-methyl-pyrimidin-2yl]sulfamoyl]benzoate

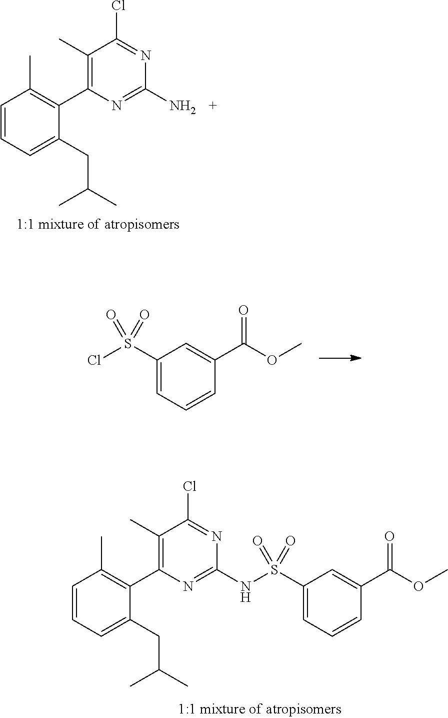 [SMILES: Cc1cccc(CC(C)C)c1-c1nc(N)nc(Cl)c1C ; COC(=O)c1cccc(S(=O)(=O)Cl)c1 ; COC(=O)c1cccc(S(=O)(=O)Nc2nc(Cl)c(C)c(-c3c(C)cccc3CC(C)C)n2)c1]

To a stirring solution of 4-chloro-6-(2-isobutyl-6-methyl-phenyl)-5-methyl-pyrimidin-2-amine (2.344 g, 8.0883 mmol) and methyl 3-chlorosulfonylbenzoate (4.812 g, 20.507 mmol) in anhydrous THF (50 mL) at −78° C. under nitrogen was added dropwise a THF solution of LiHMDS (23 mL of 1.3 M, 29.9 mmol). After the addition was complete, the reaction mixture was stirred at −78° C. for 2 h. The reaction mixture was quenched cold with 1 N aqueous HCl (100 mL), and then allowed to warm to room temperature. Ethyl acetate (120 mL) was added, and the reaction mixture was vigorously stirred for 10 min. The layers were separated, and the aqueous layer was extracted with ethyl acetate (2×100 mL). The combined organic layers were washed with brine (60 mL), dried over anhydrous sodium sulfate and concentrated. The crude was purified by silica gel chromatography using a gradient of 0-15% ethyl acetate in hexanes to afford methyl 3-[[4-chloro-6-(2-isobutyl-6-methyl-phenyl)-5-methyl-pyrimidin-2-yl]sulfamoyl]benzoate (3.791 g, 94%) as a white foam. ESI-MS m/z calc. 487.13324, found 488.4 (M+1)⁺; Retention time: 6.96 minutes; LC Method S.

### Step 8: 3-[[4-Chloro-6-(2-isobutyl-6-methyl-phenyl)-5-methyl-pyrimidin-2-yl]sulfamoyl]benzoic acid

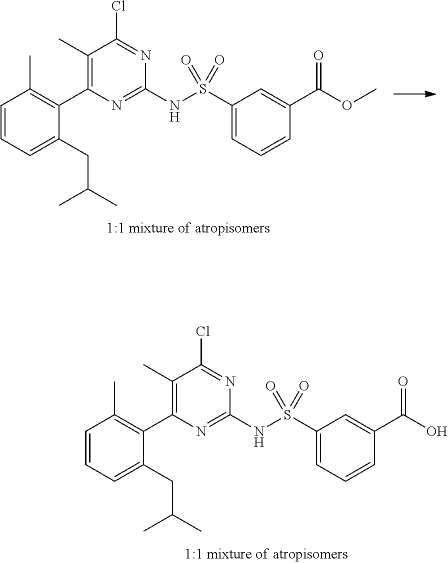 [SMILES: COC(=O)c1cccc(S(=O)(=O)Nc2nc(Cl)c(C)c(-c3c(C)cccc3CC(C)C)n2)c1 ; Cc1cccc(CC(C)C)c1-c1nc(NS(=O)(=O)c2cccc(C(=O)O)c2)nc(Cl)c1C]

To a stirring solution of methyl 3-[[4-chloro-6-(2-isobutyl-6-methyl-phenyl)-5-methyl-pyrimidin-2-yl]sulfamoyl]benzoate (3.787 g, 7.7603 mmol) in THF (65 mL) at room temperature was slowly added aqueous NaOH (40 mL of 1 M, 40.0 mmol). After the addition was complete, the reaction mixture was vigorously stirred at for 2 h. The reaction mixture was diluted with water (120 mL), and the volatiles were removed under vacuum. The residual aqueous layer was extracted with ethyl acetate (100 mL), and the organic layer was discarded. The aqueous layer was acidified with 2 M aqueous HCl to pH ˜1, and the product was extracted with ethyl acetate (250 mL). The aqueous layer was extracted with ethyl acetate (2×150 mL). The combined organic layers were evaporated to dryness to afford 3-[[4-chloro-6-(2-isobutyl-6-methyl-phenyl)-5-methyl-pyrimidin-2-yl]sulfamoyl]benzoic acid (3.54 g, 92%) as a white solid. H NMR (500 MHz, DMSO-d₆) δ 13.36 (broad s, 1H), 12.22 (broad s, 1H), 8.39 (t, J=1.9 Hz, 1H), 8.15 (d, J=7.7 Hz, 1H), 8.07 (d, J=7.8 Hz, 1H), 7.63 (t, J=7.8 Hz, 1H), 7.26 (t, J=7.6 Hz, 1H), 7.12 (d, J=7.5 Hz, 1H), 7.07 (d, J=7.6 Hz, 1H), 1.98-1.92 (m, 1H), 1.85-1.76 (m, 4H), 1.72 (s, 3H), 1.43-1.32 (m, 1H), 0.59 (d, J=6.5 Hz, 3H), 0.53 (d, J=6.5 Hz, 3H). ESI-MS m/z calc. 473.1176, found 474.1 (M+1)⁺; Retention time: 2.31 minutes; LC Method T.

### Step 9: 3-[[4-[(2R)-2-Amino-4,4-dimethyl-pentoxy]-6-(2-isobutyl-6-methyl-phenyl)-5-methyl-pyrimidin-2-yl]sulfamoyl]benzoic acid

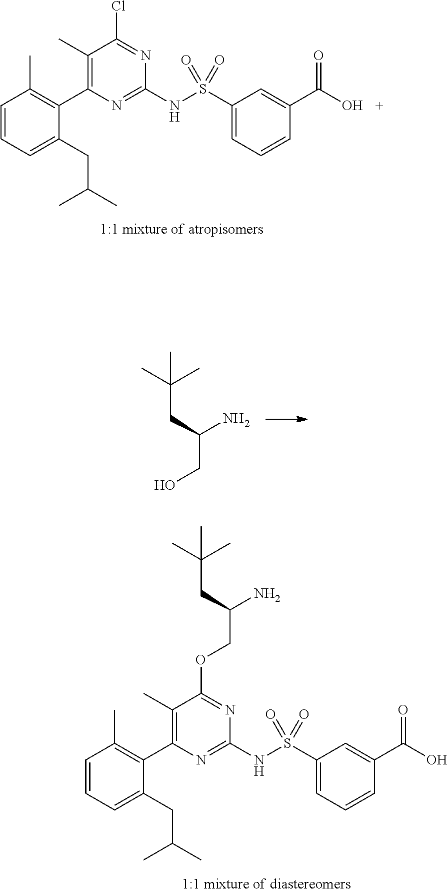

To a stirring suspension of 3-[[4-chloro-6-(2-isobutyl-6-methyl-phenyl)-5-methyl-pyrimidin-2-yl]sulfamoyl]benzoic acid (2.41 g, 5.0847 mmol) and (2R)-2-amino-4,4-dimethyl-pentan-1-ol (hydrochloride salt) (1.249 g, 7.4489 mmol) in anhydrous THF (80 mL) at room temperature under nitrogen was added sodium tert-butoxide (2.987 g, 31.081 mmol). Within 5-10 minutes, the reaction mixture became a homogeneous solution and the mixture was stirred for 2 h. The reaction mixture was cooled to 0° C., and slowly quenched with 1 N aqueous HCl (120 mL). The reaction mixture was allowed to warm to room temperature and stirred for 10 min. Ethyl acetate (150 mL) was added, and the reaction mixture was vigorously stirred for 15 min. The layers were separated, and the aqueous layer was extracted with ethyl acetate (2×100 mL). The combined organic layers were washed with brine (80 mL), dried over anhydrous sodium sulfate and concentrated. The crude was purified by reverse-phase HPLC using 40-80% acetonitrile gradient in water (5 mM HCl buffer) to afford 3-[[4-[(2R)-2-amino-4,4-dimethyl-pentoxy]-6-(2-isobutyl-6-methyl-phenyl)-5-methyl-pyrimidin-2-yl]sulfamoyl]benzoic acid (hydrochloride salt) (2.366 g, 74%) as a white solid. ¹H NMR (500 MHz, DMSO-d₆) δ 8.47-8.40 (m, 1H), 8.17-8.03 (m, 3H), 7.66 (t, J=7.8 Hz, 1H), 7.29 (broad s, 1H), 7.22-7.06 (m, 2H), 4.29 (broad s, 1H), 4.12 (broad s, 1H), 3.59 (s, 1H), 2.54 (s, 2H), 2.20-1.77 (broad m, 5H), 1.73-1.47 (m, 6H), 0.92 (d, J=10.5 Hz, 9H), 0.68 (broad s, 6H). ESI-MS m/z calc. 568.2719, found 569.5 (M+1)⁺; Retention time: 2.15 minutes; LC Method T.

### Step 10: 5-[2,2-Dimethylpropyl(methyl)amino]pyrimidine-2-carbaldehyde

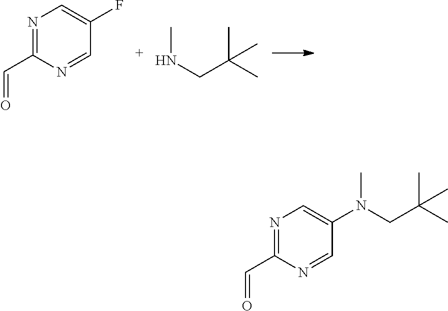

To a stirred solution of N,2,2-trimethylpropan-1-amine (hydrochloride salt) (100 mg, 0.7265 mmol) in anhydrous DMF (1.5 mL) was added 5-fluoropyrimidine-2-carbaldehyde (80 mg, 0.6345 mmol), followed by cesium carbonate (520 mg, 1.596 mmol). The heterogeneous mixture was purged with nitrogen briefly, then stirred at 110° C. for 15 h. The mixture was allowed to cool to ambient temperature. The dark reaction mixture was poured over ice-water (15 mL) and extracted with ethyl acetate (2×15 mL). The combined organics were washed sequentially with water (15 mL), brine (15 mL), dried over sodium sulfate, filtered and concentrated under reduced pressure to furnish 5-[2,2-dimethylpropyl(methyl)amino]pyrimidine-2-carbaldehyde (75 mg, 57%) as a brown solid. ¹H NMR (400 MHz, DMSO-d₆) δ 9.74 (s, 1H), 8.51 (s, 2H), 3.42 (s, 2H), 3.14 (s, 3H), 0.96 (s, 9H). ESI-MS m/z calc. 207.13716, found 208.2 (M+1)⁺; Retention time: 2.02 minutes; LC Method V.

### Step 11: (5M,11R)-11-(2,2-dimethylpropyl)-12-({5-[(2,2-dimethylpropyl)(methyl)amino]pyrimidin-2-yl}methyl)-7-methyl-6-[2-methyl-6-(2-methylpropyl)phenyl]-9-oxa-2λ⁶-thia-3,5,12,19-tetraazatricyclo[12.3.1.14,8]nonadeca-1(17),4(19),5,7,14(18),15-hexaene-2,2,13-trione (Compound 210) and (5P,11R)-11-(2,2-dimethylpropyl)-12-({5-[(2,2-dimethylpropyl)(methyl)amino]pyrimidin-2-yl}methyl)-7-methyl-6-[2-methyl-6-(2-methylpropyl)phenyl]-9-oxa-2λ⁶-thia-3,5,12,19-tetraazatricyclo[12.3.1.14,8]nonadeca-1(17),4(19),5,7,14(18),15-hexaene-2,2,13-trione (Compound 211)

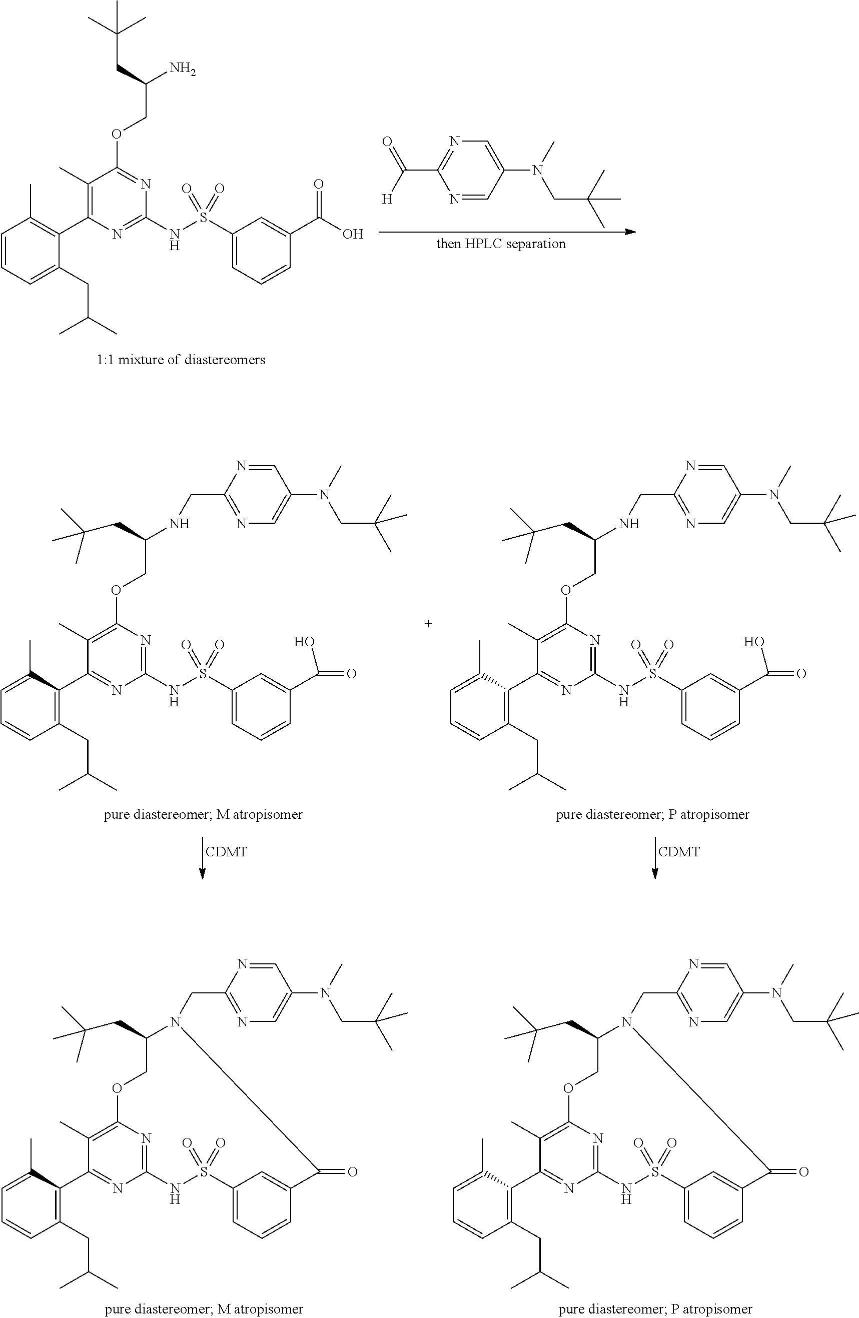

Stage 1: A 4-mL vial was charged under nitrogen with 3-[[4-[(2R)-2-amino-4,4-dimethyl-pentoxy]-6-(2-isobutyl-6-methyl-phenyl)-5-methyl-pyrimidin-2-yl]sulfamoyl]benzoic acid;hydrochloride (110 mg, 0.1818 mmol), 5-[2,2-dimethylpropyl(methyl)amino]pyrimidine-2-carbaldehyde (47 mg, 0.2268 mmol) and DCM (450 μL). The mixture was stirred at room temperature for 20 min. Sodium triacetoxyborohydride (57 mg, 0.2689 mmol) was added and the mixture was stirred at room temperature for 25 min. More sodium triacetoxyborohydride (142 mg, 0.670 mmol) was added and the mixture was stirred at room temperature for 2.5 h. The reaction was quenched with a minimum amount of 1 N aqueous HCl. Methanol and DMSO were added. After filtration, purification by reverse-phase HPLC (1-99% acetonitrile/5 mM HCl) provided two isomers. First to elute, more polar isomer: 3-{[(1M)-4-{[(2R)-2-[({5-[(2,2-dimethylpropyl)(methyl)amino]pyrimidin-2-yl}methyl)amino]-4,4-dimethylpentyl]oxy}-5-methyl-6-[2-methyl-6-(2-methylpropyl)phenyl]pyrimidin-2-yl]sulfamoyl}benzoic acid (hydrochloride salt) (29.6 mg, 41%). ESI-MS m/z calc. 759.4142, found 760.53 (M+1)⁺; Retention time: 1.8 minutes; LC Method A. Second to elute, less polar isomer: 3-{[(1P)-4-{[(2R)-2-[({5-[(2,2-dimethylpropyl)(methyl)amino]pyrimidin-2-yl}methyl)amino]-4,4-dimethylpentyl]oxy}-5-methyl-6-[2-methyl-6-(2-methylpropyl)phenyl]pyrimidin-2-yl]sulfamoyl}benzoic acid (hydrochloride salt) (20 mg, 28%). ESI-MS m/z calc. 759.4142, found 760.61 (M+1)⁺; Retention time: 1.82 minutes; LC Method A.

Stage 2: Each isomer was mixed under nitrogen in a 4-mL vial with CDMT (19 mg, 0.1082 mmol) and anhydrous DMF (1.3 mL). The solution was stirred at 0° C. 4-Methylmorpholine (40 μL, 0.3638 mmol) was added and the mixture was stirred in the cooling bath, which was allowed to warm to room temperature over 18 h. Thereafter, the reaction was diluted with DMSO and the solution was filtered. Purification by reverse-phase HPLC (10-99% acetonitrile/5 mM HCl) provided two separate isomers. Originating from more polar isomer: (5M,11R)-11-(2,2-dimethylpropyl)-12-({5-[(2,2-dimethylpropyl)(methyl)amino]pyrimidin-2-yl}methyl)-7-methyl-6-[2-methyl-6-(2-methylpropyl)phenyl]-9-oxa-2λ⁶-thia-3,5,12,19-tetraazatricyclo[12.3.1.14,8]nonadeca-1(17),4(19),5,7,14(18),15-hexaene-2,2,13-trione (13.5 mg, 20%). ¹H NMR (400 MHz, DMSO-d₆) δ 13.32-11.68 (broad m, 1H), 8.69 (br s, 1H), 8.32 (s, 2H), 7.91 (br s, 1H), 7.65 (br s, 2H), 7.32 (br s, 1H), 7.19 (d, J=7.5 Hz, 1H), 7.16-6.91 (m, 1H), 5.55-5.24 (m, 1H), 4.84 (d, J=16.2 Hz, 1H), 4.55 (d, J=16.3 Hz, 1H), 4.14 (t, J=11.1 Hz, 1H), 4.06-3.91 (m, 1H), 3.26 (d, J=15.0 Hz, 1H), 3.20 (d, J=15.0 Hz, 1H), 3.00 (s, 3H), 2.13-2.02 (m, 3H), 2.00-1.90 (m, 2H), 1.85 (dd, J=15.2, 9.3 Hz, 1H), 1.71-1.49 (m, 4H), 1.34 (d, J=15.1 Hz, 1H), 0.94 (s, 9H), 0.70-0.49 (m, 15H). ESI-MS m/z calc. 741.4036, found 742.75 (M+1)⁺; Retention time: 2.26 minutes; LC Method A. Originating from less polar isomer: (5P,11R)-11-(2,2-dimethylpropyl)-12-({5-[(2,2-dimethylpropyl)(methyl)amino]pyrimidin-2-yl}methyl)-7-methyl-6-[2-methyl-6-(2-methylpropyl)phenyl]-9-oxa-2λ⁶-thia-3,5,12,19-tetraazatricyclo[12.3.1.14,8]nonadeca-1(17),4(19),5,7,14(18),15-hexaene-2,2,13-trione (7.7 mg, 11%). ¹H NMR (400 MHz, DMSO-d₆) δ 13.19-11.49 (broad m, 1H), 8.70 (br s, 1H), 8.31 (s, 2H), 7.93 (br s, 1H), 7.66 (br s, 2H), 7.31 (br s, 1H), 7.21-7.07 (m, 2H), 5.50-5.34 (m, 1H), 4.86 (d, J=16.2 Hz, 1H), 4.54 (d, J=16.2 Hz, 1H), 4.16 (t, J=11.2 Hz, 1H), 4.06-3.96 (m, 1H), 3.26 (d, J=15.0 Hz, 1H), 3.20 (d, J=15.0 Hz, 1H), 3.00 (s, 3H), 2.21 (d, J=7.5 Hz, 2H), 1.87-1.68 (m, 5H), 1.58 (s, 3H), 1.36 (d, J=15.1 Hz, 1H), 0.95 (s, 9H), 0.85-0.71 (m, 6H), 0.54 (s, 9H). ESI-MS m/z calc. 741.4036, found 742.71 (M+1)⁺; Retention time: 2.29 minutes; LC Method A.

### Example 73: Preparation of (Compound 212) and (Compound 213)

### Step 1: 4-Chloro-6-(2-isobutyl-6-methyl-phenyl)-5-(trifluoromethyl)pyrimidin-2-amine

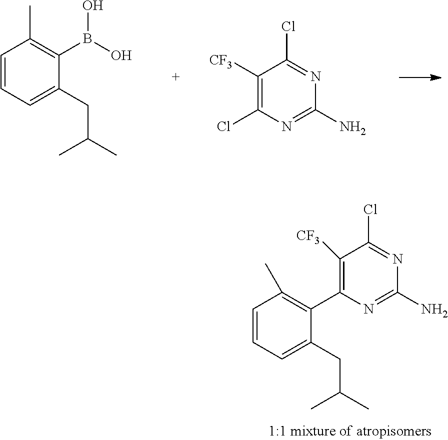 [SMILES: Cc1cccc(CC(C)C)c1B(O)O ; Nc1nc(Cl)c(C(F)(F)F)c(Cl)n1 ; Cc1cccc(CC(C)C)c1-c1nc(N)nc(Cl)c1C(F)(F)F]

A stirring solution of (2-isobutyl-6-methyl-phenyl)boronic acid (0.96 g, 4.9984 mmol) and 4,6-dichloro-5-(trifluoromethyl)pyrimidin-2-amine (1.226 g, 5.2847 mmol) in anhydrous toluene (22 mL) at room temperature was degassed with nitrogen for 15 min. Under nitrogen, K₃PO₄ (3.191 g, 15.033 mmol), 3-(t-butyl)-4-(2,6-dimethoxyphenyl)-2,3-dihydrobenzo[d][1,3]oxaphosphole (rac-BI-DIME, 168 mg, 0.5085 mmol) and Pd₂(dba)₃ (234 mg, 0.2555 mmol) were added. The reaction mixture was heated at 110° C. for 6 h. After cooling to room temperature, water (50 mL) and ethyl acetate (40 mL) were added, and the reaction mixture was vigorously stirred for 10 min. The layers were separated, and the aqueous layer was extracted with ethyl acetate (2×30 mL). The combined organic layers were washed with brine (20 mL), dried over anhydrous sodium sulfate and concentrated. The crude was purified by silica gel chromatography using 0-10% ethyl acetate gradient in hexanes, followed by reverse-phase HPLC using 55-100% acetonitrile gradient in water (0.1% TFA buffer). The pure product fractions were combined and basified with saturated aqueous NaHCO₃ to pH ˜7. The volatiles were removed in vacuo, and the residual aqueous layer was extracted with ethyl acetate (3×25 mL). The combined organic layers were washed with brine (20 mL), dried over anhydrous sodium sulfate and concentrated to afford 4-chloro-6-(2-isobutyl-6-methyl-phenyl)-5-(trifluoromethyl)pyrimidin-2-amine (398 mg, 22%) as a white solid. ESI-MS m/z calc. 343.10632, found 344.4 (M+1)⁺; Retention time: 6.36 minutes; LC Method S.

### Step 2: Methyl 3-[[4-chloro-6-(2-isobutyl-6-methyl-phenyl)-5-(trifluoromethyl)pyrimidin-2-yl]sulfamoyl]benzoate

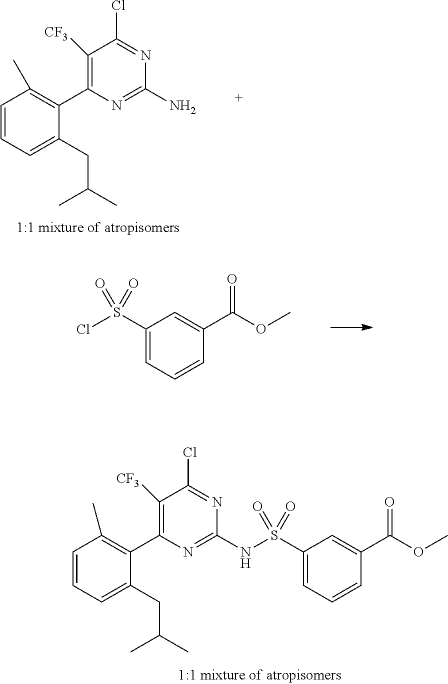 [SMILES: Cc1cccc(CC(C)C)c1-c1nc(N)nc(Cl)c1C(F)(F)F ; COC(=O)c1cccc(S(=O)(=O)Cl)c1 ; COC(=O)c1cccc(S(=O)(=O)Nc2nc(Cl)c(C(F)(F)F)c(-c3c(C)cccc3CC(C)C)n2)c1]

To a stirring solution of 4-chloro-6-(2-isobutyl-6-methyl-phenyl)-5-(trifluoromethyl)pyrimidin-2-amine (398 mg, 1.1577 mmol) and methyl 3-chlorosulfonylbenzoate (686 mg, 2.9234 mmol) in anhydrous 2-MeTHF (7 mL) at 0° C. under nitrogen was added dropwise a heptanes solution of lithium tert-amoxide (963.60 mg, 3.3 mL of 40% w/w, 4.0969 mmol). After the addition was complete, the reaction mixture was allowed to warm to room temperature and stirred for 2 h. The reaction mixture was cooled to 0° C., and slowly quenched with ice-cold 1 N aqueous HCl (12 mL). The reaction mixture was allowed to warm to room temperature, ethyl acetate (25 mL) was added, and the reaction mixture was vigorously stirred for 5 min. The layers were separated, and the aqueous layer was extracted with ethyl acetate (2×20 mL). The combined organic layers were washed with brine (20 mL), dried over anhydrous sodium sulfate and concentrated. The crude was purified by reverse-phase HPLC using 60-100% acetonitrile gradient in water (0.1% TFA buffer). All pure product fractions were combined, and volatiles were removed under vacuum. The residual aqueous layer was extracted with ethyl acetate (3×25 mL). The combined organic layers were washed with brine (20 mL), dried over anhydrous sodium sulfate and concentrated to afford methyl 3-[[4-chloro-6-(2-isobutyl-6-methyl-phenyl)-5-(trifluoromethyl)pyrimidin-2-yl]sulfamoyl]benzoate (369 mg, 55%) as a white solid. ESI-MS m/z calc. 541.105, found 542.5 (M+1)⁺; Retention time: 6.97 minutes; LC Method S.

### Step 3: 3-[[4-Chloro-6-(2-isobutyl-6-methyl-phenyl)-5-(trifluoromethyl)pyrimidin-2-yl]sulfamoyl]benzoic acid

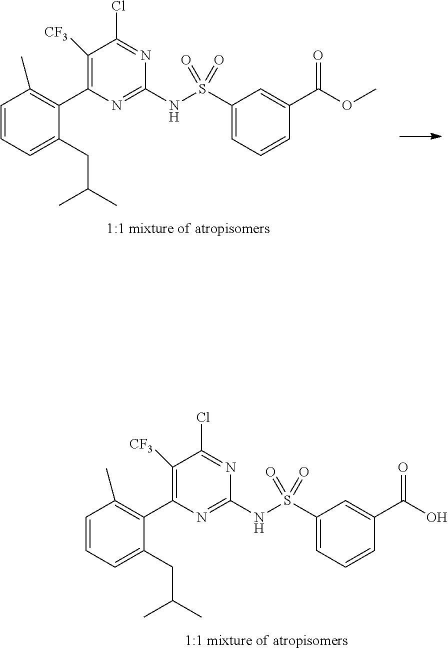 [SMILES: COC(=O)c1cccc(S(=O)(=O)Nc2nc(Cl)c(C(F)(F)F)c(-c3c(C)cccc3CC(C)C)n2)c1 ; Cc1cccc(CC(C)C)c1-c1nc(NS(=O)(=O)c2cccc(C(=O)O)c2)nc(Cl)c1C(F)(F)F]

To a stirring solution of methyl 3-[[4-chloro-6-(2-isobutyl-6-methyl-phenyl)-5-(trifluoromethyl)pyrimidin-2-yl]sulfamoyl]benzoate (344 mg, 0.6347 mmol) in THF (6 mL) at room temperature was slowly added aqueous lithium hydroxide monohydrate (2.6 mL of 1 M, 2.60 mmol). After the addition was complete, the reaction mixture was stirred at this temperature for 5 h. The reaction mixture was cooled to 0° C., slowly quenched with saturated aqueous NH₄Cl (10 mL), and then slowly acidified with ice-cold 1 N aqueous HCl (5 mL). Ethyl acetate (15 mL) was added, and the vigorously stirred reaction mixture was allowed to warm to room temperature. The layers were separated, and the aqueous layer was extracted with ethyl acetate (2×10 mL). The combined organic layers were washed with brine (10 mL), dried over anhydrous sodium sulfate and concentrated to afford 3-[[4-chloro-6-(2-isobutyl-6-methyl-phenyl)-5-(trifluoromethyl)pyrimidin-2-yl]sulfamoyl]benzoic acid (338 mg, 91%) as a white solid. ESI-MS m/z calc. 527.08936, found 528.3 (M+1)⁺; Retention time: 6.15 minutes; LC Method S.

### Step 4: 3-[[4-[(2R)-2-Amino-4,4-dimethyl-pentoxy]-6-(2-isobutyl-6-methyl-phenyl)-5-(trifluoromethyl)pyrimidin-2-yl]sulfamoyl]benzoic acid

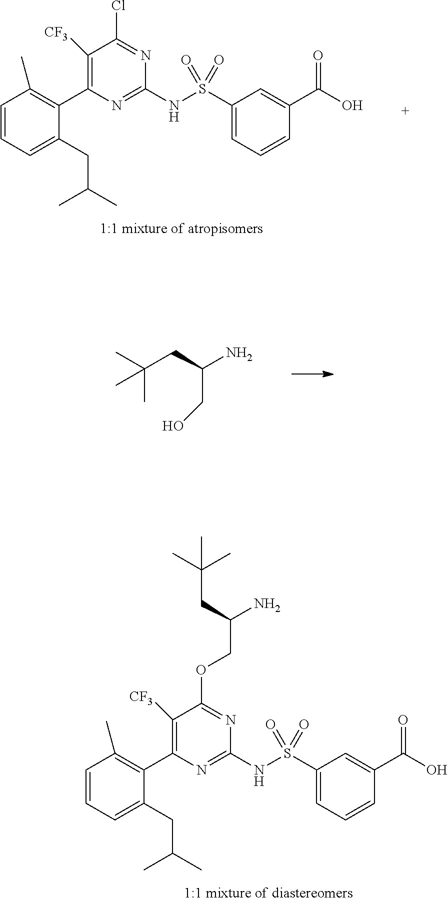

To a stirring, pre-sonicated suspension of 3-[[4-chloro-6-(2-isobutyl-6-methyl-phenyl)-5-(trifluoromethyl)pyrimidin-2-yl]sulfamoyl]benzoic acid (302 mg, 0.5720 mmol) and (2R)-2-amino-4,4-dimethyl-pentan-1-ol (hydrochloride salt) (132 mg, 0.7872 mmol) in anhydrous THF (5.6 mL) at room temperature under nitrogen was added sodium tert-butoxide (292 mg, 3.0384 mmol) in one portion. Within 5 min, the reaction mixture became an opaque, homogeneous solution. The reaction mixture was stirred at room temperature for 2 h. The reaction mixture was cooled to 0° C., and slowly quenched with 1 N aqueous HCl (7 mL). The reaction mixture was allowed to warm to room temperature, ethyl acetate (15 mL) was added, and the reaction mixture was vigorously stirred for 5 min. The layers were separated, and the aqueous layer was extracted with ethyl acetate (2×15 mL). The combined organic layers were washed with brine (10 mL), dried over anhydrous sodium sulfate and concentrated. The crude was purified by reverse-phase HPLC using 30-70% acetonitrile gradient in water (5 mM HCl buffer) to afford 3-[[4-[(2R)-2-amino-4,4-dimethyl-pentoxy]-6-(2-isobutyl-6-methyl-phenyl)-5-(trifluoromethyl)pyrimidin-2-yl]sulfamoyl]benzoic acid (hydrochloride salt) (313 mg, 80%) as a white solid. ¹H NMR (500 MHz, DMSO-d₆) δ 13.45 (broad s, 1H), 12.81 (broad s, 1H), 8.39 (s, 1H), 8.24-8.08 (m, 5H), 7.68 (t, J=7.8 Hz, 1H), 7.25 (t, J=7.6 Hz, 1H), 7.12-7.03 (m, 2H), 4.60-4.48 (m, 1H), 4.42-4.32 (m, 1H), 3.64 (s, 1H), 2.11-2.00 (m, 1H), 2.00-1.89 (m, 1H), 1.82 (apparent d, J=24.1 Hz, 3H), 1.71-1.51 (m, 3H), 0.91 (d, J=5.7 Hz, 9H), 0.73-0.57 (m, 6H). ESI-MS m/z calc. 622.24365, found 623.5 (M+1)⁺; Retention time: 1.94 minutes; LC Method T.

### Step 5: 3-[[4-[4,4-Dimethyl-2-[(5-morpholinopyrimidin-2-yl)methylamino]pentoxy]-6-(2-isobutyl-6-methyl-phenyl)-5-(trifluoromethyl)pyrimidin-2-yl]sulfamoyl]benzoic acid (hydrochloride salt)

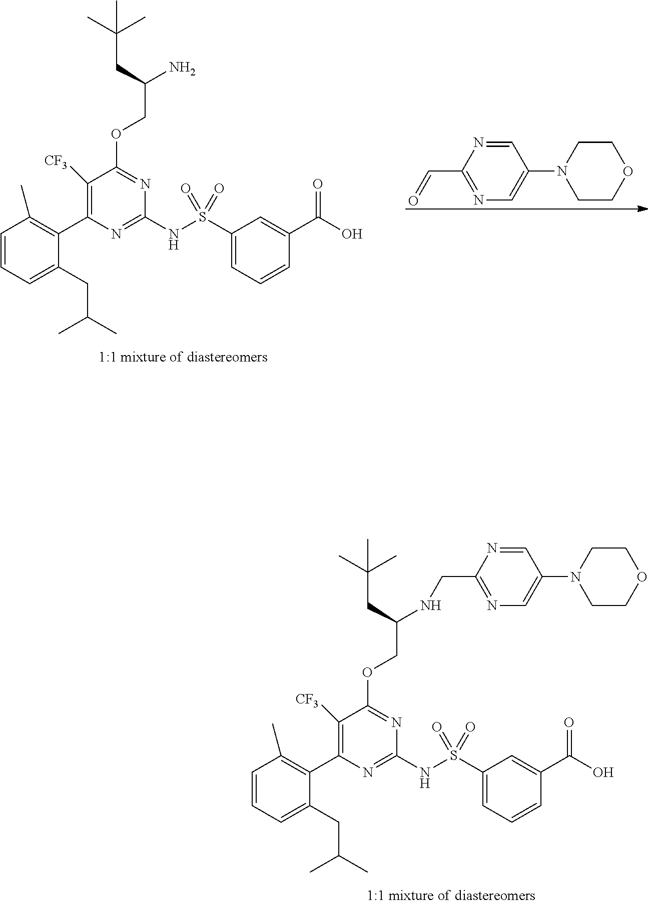 [SMILES: Cc1cccc(CC(C)C)c1-c1nc(NS(=O)(=O)c2cccc(C(=O)O)c2)nc(OC[C@H](N)CC(C)(C)C)c1C(F)(F)F ; O=Cc1ncc(N2CCOCC2)cn1 ; Cc1cccc(CC(C)C)c1-c1nc(NS(=O)(=O)c2cccc(C(=O)O)c2)nc(OC[C@@H](CC(C)(C)C)NCc2ncc(N3CCOCC3)cn2)c1C(F)(F)F]

To a stirred mixture of 3-[[4-(2-amino-4,4-dimethyl-pentoxy)-6-(2-isobutyl-6-methyl-phenyl)-5-(trifluoromethyl)pyrimidin-2-yl]sulfamoyl]benzoic acid (hydrochloride salt) (100 mg, 0.1430 mmol) and 5-morpholinopyrimidine-2-carbaldehyde (hydrochloride salt) (47 mg, 0.2046 mmol) in DCM (1 mL) was added sodium triacetoxyborohydride (90 mg, 0.4034 mmol) at rt and stirred for 3 h. More sodium triacetoxyborohydride (58 mg, 0.2600 mmol) was added and stirred for 1 h. The reaction mixture was quenched with 1 N HCl solution (0.5 mL), diluted with DCM (5 mL) and filtered. The filtrate was concentrated in vacuo and the resulting red oil was purified by reverse-phase HPLC (C₁₈ column, 30-90% MeCN in water) to give 3-[[4-[4,4-dimethyl-2-[(5-morpholinopyrimidin-2-yl)methylamino]pentoxy]-6-(2-isobutyl-6-methyl-phenyl)-5-(trifluoromethyl)pyrimidin-2-yl]sulfamoyl]benzoic acid (hydrochloride salt) (69 mg, 58%) as a beige solid (mixture of isomers). ESI-MS m/z calc. 799.3339, found 800.8 (M+1)⁺; Retention time: 6.27 minutes. LC Method S.

### Step 6: (11R)-11-(2,2-Dimethylpropyl)-6-(2-isobutyl-6-methyl-phenyl)-12-[(5-morpholinopyrimidin-2-yl)methyl]-2,2-dioxo-7-(trifluoromethyl)-9-oxa-2λ⁶-thia-3,5,12,19-tetrazatricyclo[12.3.1.14,8]nonadeca-1(18),4(19),5,7,14,16-hexaen-13-one

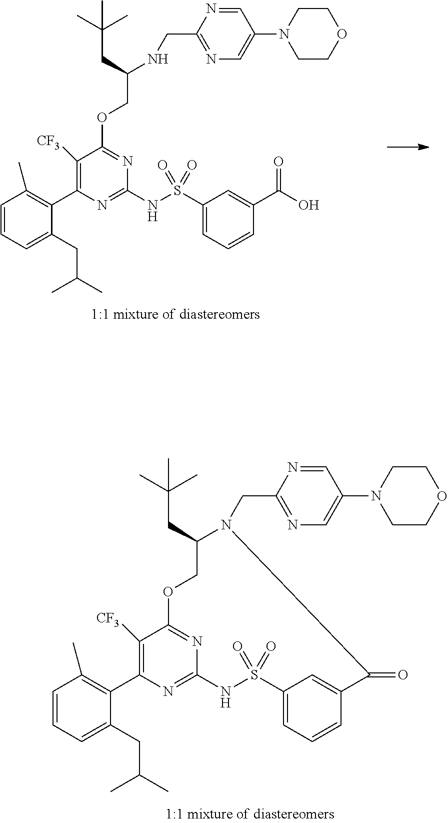 [SMILES: Cc1cccc(CC(C)C)c1-c1nc(NS(=O)(=O)c2cccc(C(=O)O)c2)nc(OC[C@@H](CC(C)(C)C)NCc2ncc(N3CCOCC3)cn2)c1C(F)(F)F ; Cc1cccc(CC(C)C)c1-c1nc2nc(c1C(F)(F)F)OC[C@@H](CC(C)(C)C)N(Cc1ncc(N3CCOCC3)cn1)C(=O)c1cccc(c1)S(=O)(=O)N2]

A solution of 3-[[4-[(2R)-4,4-dimethyl-2-[(5-morpholinopyrimidin-2-yl)methylamino]pentoxy]-6-(2-isobutyl-6-methyl-phenyl)-5-(trifluoromethyl)pyrimidin-2-yl]sulfamoyl]benzoic acid (hydrochloride salt) (69 mg, 0.0916 mmol), CDMT (25 mg, 0.1424 mmol) and N-methylmorpholine (46.0 mg, 50 μL, 0.4548 mmol) in DMF (1 mL) was stirred at rt for 2 h. Another portion of CDMT (25 mg, 0.1424 mmol) and N-methylmorpholine (46.0 mg, 50 μL, 0.4548 mmol) was added and the reaction mixture was stirred for 2 h. The reaction mixture was purified by reverse-phase HPLC (C18 column, 30-90% MeCN in water with 0.1% HCl modifier) to give (11R)-11-(2,2-dimethylpropyl)-6-(2-isobutyl-6-methyl-phenyl)-12-[(5-morpholinopyrimidin-2-yl)methyl]-2,2-dioxo-7-(trifluoromethyl)-9-oxa-2λ⁶-thia-3,5,12,19-tetrazatricyclo[12.3.1.14,8]nonadeca-1(18),4(19),5,7,14,16-hexaen-13-one (hydrochloride salt) (29.2 mg, 37%) as a white solid (mixture of isomers). ¹H NMR (500 MHz, DMSO-d₆) δ 13.03 (s, 1H), 8.71 (d, J=3.4 Hz, 1H), 8.48 (d, J=2.4 Hz, 2H), 8.05-7.97 (m, 1H), 7.80-7.74 (m, 2H), 7.27 (dd, J=7.6, 7.6 Hz, 1H), 7.13 (dd, J=7.8, 3.4 Hz, 1H), 7.07 (dd, J=7.8, 4.0 Hz, 1H), 5.50 (dd, J=10.9, 4.4 Hz, 1H), 4.83 (dd, J=16.4, 9.1 Hz, 1H), 4.66 (dd, J=16.6, 2.9 Hz, 1H), 4.40-4.26 (m, 1H), 3.97 (dd, J=18.8, 10.8 Hz, 1H), 3.74 (dd, J=4.9, 4.9 Hz, 4H), 3.22 (dd, J=4.9, 4.9 Hz, 4H), 2.27-2.11 (m, 1H), 2.03 (s, 2H), 1.98 (dd, J=13.9, 7.7 Hz, 1H), 1.85 (dd, J=14.3, 8.1 Hz, 1H), 1.76 (s, 1H), 1.65-1.54 (m, 1H), 1.39 (d, J=15.1 Hz, 1H), 0.78 (dd, J=6.6, 2.8 Hz, 3H), 0.62 (d, J=6.6 Hz, 2H), 0.59 (s, 5H), 0.56 (s, 4H), 0.52 (d, J=6.6 Hz, 2H). ESI-MS m/z calc. 781.3233, found 782.8 (M+1)⁺; Retention time: 3.1 minutes; LC Method W.

### Step 7: (11R)-11-(2,2-dimethylpropyl)-6-(2-isobutyl-6-methyl-phenyl)-12-[(5-morpholinopyrimidin-2-yl)methyl]-2,2-dioxo-7-(trifluoromethyl)-9-oxa-2λ⁶-thia-3,5,12,19-tetrazatricyclo[12.3.1.14,8]nonadeca-1(18),4(19),5,7,14,16-hexaen-13-one (Compound 212) and (11R)-11-(2,2-dimethylpropyl)-6-(2-isobutyl-6-methyl-phenyl)-12-[(5-morpholinopyrimidin-2-yl)methyl]-2,2-dioxo-7-(trifluoromethyl)-9-oxa-2λ⁶-thia-3,5,12,19-tetrazatricyclo[12.3.1.14,8]nonadeca-1(18),4(19),5,7,14,16-hexaen-13-one (Compound 213)

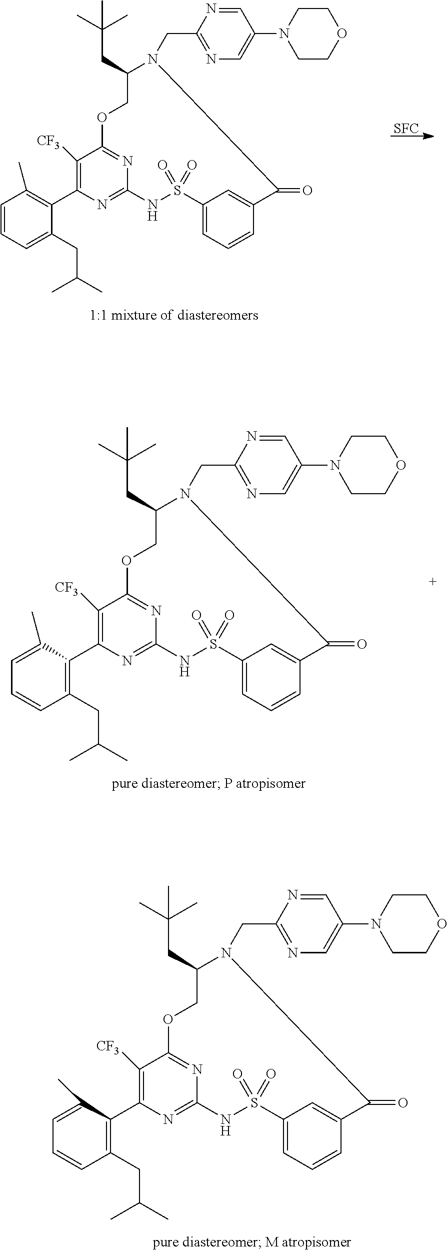

The two isomers of (11R)-11-(2,2-dimethylpropyl)-6-(2-isobutyl-6-methyl-phenyl)-12-[(5-morpholinopyrimidin-2-yl)methyl]-2,2-dioxo-7-(trifluoromethyl)-9-oxa-2λ⁶-thia-3,5,12,19-tetrazatricyclo[12.3.1.14,8]nonadeca-1(18),4(19),5,7,14,16-hexaen-13-one (28.3 mg, 0.0355 mmol) were separated by chiral SFC using method: Lux i-Cellulose-5, 250×21.2 mm, 5 m, column temp=40° C.; flow rate 75 mL/min, 45% MeOH+55% CO₂. The first eluting compound was the P isomer, (11R)-11-(2,2-dimethylpropyl)-6-(2-isobutyl-6-methyl-phenyl)-12-[(5-morpholinopyrimidin-2-yl)methyl]-2,2-dioxo-7-(trifluoromethyl)-9-oxa-2λ⁶-thia-3,5,12,19-tetrazatricyclo[12.3.1.14,8]nonadeca-1(18),4(19),5,7,14,16-hexaen-13-one (8.3 mg, 60%) as a white solid; ¹H NMR (400 MHz, DMSO-d₆) δ 13.05 (br s, 1H), 8.72 (s, 1H), 8.48 (s, 2H), 8.07-7.97 (m, 1H), 7.83-7.72 (m, 2H), 7.27 (t, J=7.6 Hz, 1H), 7.13 (d, J=7.8 Hz, 1H), 7.08 (d, J=7.6 Hz, 1H), 5.51 (dd, J=11.0, 4.4 Hz, 1H), 4.85 (d, J=16.6 Hz, 1H), 4.66 (d, J=16.6 Hz, 1H), 4.33 (t, J=11.0 Hz, 1H), 4.09-3.97 (m, 1H), 3.79-3.71 (m, 4H), 3.26-3.19 (m, 4H), 2.28-2.14 (m, 2H), 1.89-1.73 (m, 5H), 1.40 (br d, J=14.9 Hz, 1H), 0.80 (d, J=2.2 Hz, 3H), 0.78 (d, J=2.2 Hz, 3H), 0.57 (s, 9H). ¹⁹F NMR (377 MHz, DMSO-d₆) δ −56.61 (br s, 3F). ESI-MS m/z calc. 781.3233, found 782.4 (M+1)⁺; Retention time: 3.13 minutes; LC Method 1D. The second eluting compound was the M isomer, (11R)-11-(2,2-dimethylpropyl)-6-(2-isobutyl-6-methyl-phenyl)-12-[(5-morpholinopyrimidin-2-yl)methyl]-2,2-dioxo-7-(trifluoromethyl)-9-oxa-2λ⁶-thia-3,5,12,19-tetrazatricyclo[12.3.1.14,8]nonadeca-1(18),4(19),5,7,14,16-hexaen-13-one (8.7 mg, 63%) as a white solid. ¹H NMR (400 MHz, DMSO-d₆) δ 13.07 (br s, 1H), 8.71 (s, 1H), 8.49 (s, 2H), 7.99 (br s, 1H), 7.76 (br s, 2H), 7.33-7.21 (m, 1H), 7.13 (d, J=6.8 Hz, 1H), 7.06 (d, J=7.3 Hz, 1H), 5.56-5.43 (m, 1H), 4.83 (d, J=16.6 Hz, 1H), 4.68 (d, J=15.7 Hz, 1H), 4.34 (br s, 1H), 4.00 (br s, 1H), 3.78-3.72 (m, 4H), 3.27-3.20 (m, 4H), 2.04 (s, 3H), 2.02-1.94 (m, 1H), 1.92-1.78 (m, 2H), 1.68-1.56 (m, 1H), 1.41 (br d, J=15.2 Hz, 1H), 0.63 (d, J=6.4 Hz, 3H), 0.60 (s, 9H), 0.53 (d, J=6.4 Hz, 3H). ¹⁹F NMR (377 MHz, DMSO-d₆) δ −56.65 (br s, 3F). ESI-MS m/z calc. 781.3233, found 782.3 (M+1)⁺; Retention time: 3.11 minutes; LC Method 1D.

### Example 74: Preparation of Compound 214 and Compound 215

### Step 1: 2-Bromo-1-(2,2-dimethylpropyl)-3-methyl-benzene

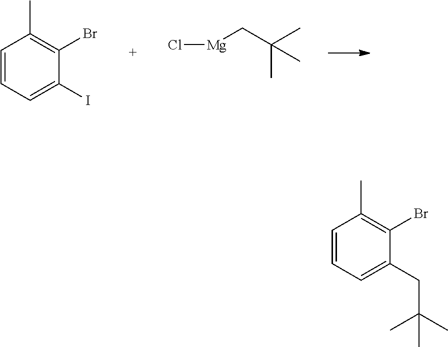

To a stirred solution of 2-bromo-1-iodo-3-methyl-benzene (10 g, 33.678 mmol) in dry dioxane (300 mL) at room temperature under nitrogen atmosphere were successively added Pd(amphos)Cl₂ (2.4 g, 3.3799 mmol) and a solution of chloro(2,2-dimethylpropyl)magnesium in tetrahydrofuran (44 mL of 1 M, 44.0 mmol). The reaction was stirred overnight at 30° C. Additional Pd(amphos)Cl₂ (1.2 g, 1.6899 mmol) and chloro(2,2-dimethylpropyl)magnesium in THE (22 mL of 1 M, 22.000 mmol) were added and the reaction was stirred overnight at 30° C. Then, aqueous 6 N HCl solution (250 mL) was added and the aqueous layer was extracted with pentane (5×250 mL). The combined organic layers were washed with water (500 mL), brine (500 mL) and dried over sodium sulfate. The organic layer then filtered on a pad of silica gel and washed with pentane (3×100 mL). The filtrate was concentrated under reduced pressure to afford crude 2-bromo-1-(2,2-dimethylpropyl)-3-methyl-benzene (11.06 g, 57%) as a pale brown oil.

### Step 2: [2-(2,2-Dimethylpropyl)-6-methyl-phenyl]boronic acid

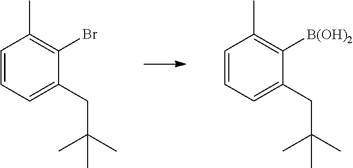

To a stirred solution of 2-bromo-1-(2,2-dimethylpropyl)-3-methyl-benzene (5.35 g, 22.184 mmol) in dry THF (350 mL) under nitrogen atmosphere cooled to −78° C. was added a solution of n-butyllithium in hexanes (10.7 mL of 2.5 M, 26.75 mmol). The reaction was stirred for 1 h at −78° C., after which trimethylborate (3.0030 g, 3.5 mL, 28.899 mmol) was added. The reaction was stirred for 45 min at −78° C. then allowed to warm to room temperature, then stirred at room temperature for 45 min. An aqueous solution of 1 N HCl (150 mL) was added and the reaction was vigorously stirred at room temperature for 30 min. The aqueous layer was extracted with dichloromethane (4×150 mL) and the combined extracts were washed with water (250 mL), brine (250 mL), dried over sodium sulfate, filtered and concentrated under reduced pressure to afford a pale yellow oil. This material was triturated with heptane (100 mL), filtered and dried under high vacuum to afford [2-(2,2-dimethylpropyl)-6-methyl-phenyl]boronic acid (2.253 g, 49%) as a white solid. ¹H NMR (400 MHz, DMSO-d₆) δ 8.01 (s, 2H), 7.08 (t, J=6.8 Hz, 1H), 6.94 (d, J=7.3 Hz, 1H), 6.90 (d, J=7.3 Hz, 1H), 2.54 (s, 2H), 2.29 (s, 3H), 0.88 (s, 9H). ESI-MS m/z calc. 206.14781, found 205.3 (M−1)⁻; Retention time: 3.1 minutes; LC Method 1D.

### Step 3: tert-Butyl N-tert-butoxycarbonyl-N-[4-chloro-6-[2-(2,2-dimethylpropyl)-6-methyl-phenyl]-5-methyl-pyrimidin-2-yl]carbamate

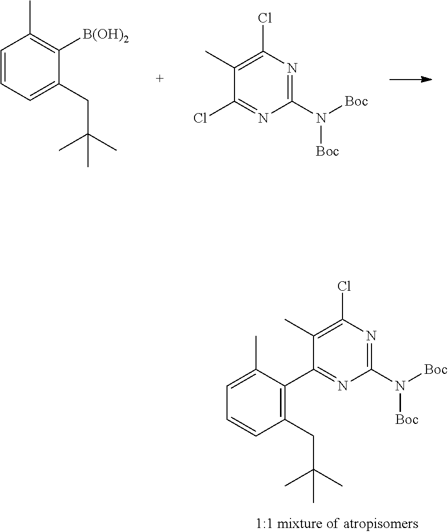 [SMILES: Cc1cccc(CC(C)(C)C)c1B(O)O ; Cc1c(Cl)nc(N(C(=O)OC(C)(C)C)C(=O)OC(C)(C)C)nc1Cl ; Cc1cccc(CC(C)(C)C)c1-c1nc(N(C(=O)OC(C)(C)C)C(=O)OC(C)(C)C)nc(Cl)c1C]

A reaction vial was charged with [2-(2,2-dimethylpropyl)-6-methyl-phenyl]boronic acid (1.09 g, 5.2890 mmol), tert-butyl N-tert-butoxycarbonyl-N-(4,6-dichloro-5-methyl-pyrimidin-2-yl)carbamate (2.412 g, 6.3767 mmol) and cesium carbonate (5.125 g, 15.730 mmol) in a solvent mixture of dimethoxyethane (9 mL) and water (3 mL). The reaction mixture was purged with argon for 3 min. Pd(dppf)Cl₂ (393 mg, 0.5371 mmol) was added to the reaction mixture. The reaction was stirred for 4 h. The reaction was diluted with ethyl acetate (30 mL) and washed with brine (20 mL). The organic layer was dried over anhydrous sodium sulfate and concentrated under vacuum. The residue was purified by reverse-phase HPLC using 80-100% acetonitrile in water (buffered with 0.1% TFA) to furnish tert-butyl N-tert-butoxycarbonyl-N-[4-chloro-6-[2-(2,2-dimethylpropyl)-6-methyl-phenyl]-5-methyl-pyrimidin-2-yl]carbamate (0.598 g, 21%). ESI-MS m/z calc. 503.2551, found 504.4 (M+1)⁺; Retention time: 4.58 minutes; LC Method T.

### Step 4: 4-Chloro-6-[2-(2,2-dimethylpropyl)-6-methyl-phenyl]-5-methyl-pyrimidin-2-amine

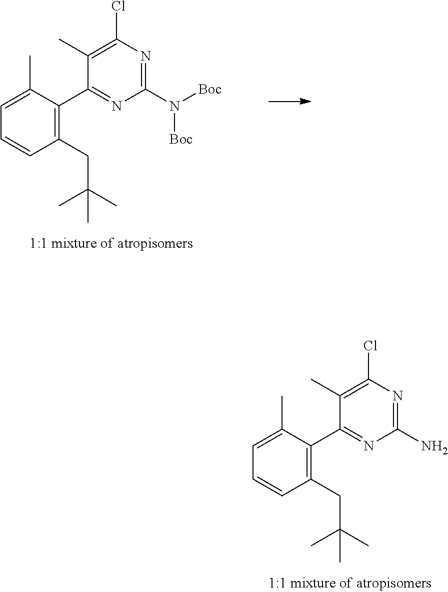 [SMILES: Cc1cccc(CC(C)(C)C)c1-c1nc(N(C(=O)OC(C)(C)C)C(=O)OC(C)(C)C)nc(Cl)c1C ; Cc1cccc(CC(C)(C)C)c1-c1nc(N)nc(Cl)c1C]

To a solution of tert-butyl N-tert-butoxycarbonyl-N-[4-chloro-6-[2-(2,2-dimethylpropyl)-6-methyl-phenyl]-5-methyl-pyrimidin-2-yl]carbamate (1.142 g, 2.2656 mmol) in anhydrous DCM (12 mL) was added 2.0 N HCl in ether (12 mL of 2 M, 24.0 mmol). The reaction was stirred at rt for 2 days. The solvents were removed under vacuum. The residue was diluted with DCM (50 mL) and washed with saturated sodium bicarbonate (30 mL), and brine (30 mL). The DCM layer was dried over anhydrous sodium sulfate and concentrated under vacuum to furnish 4-chloro-6-[2-(2,2-dimethylpropyl)-6-methyl-phenyl]-5-methyl-pyrimidin-2-amine (693 mg, 96%) as a white solid. ESI-MS m/z calc. 303.15024, found 304.2 (M+1)⁺; Retention time: 3.57 minutes; LC Method T.

### Step 5: Methyl 3-[[4-chloro-6-[2-(2,2-dimethylpropyl)-6-methyl-phenyl]-5-methyl-pyrimidin-2-yl]sulfamoyl]benzoate

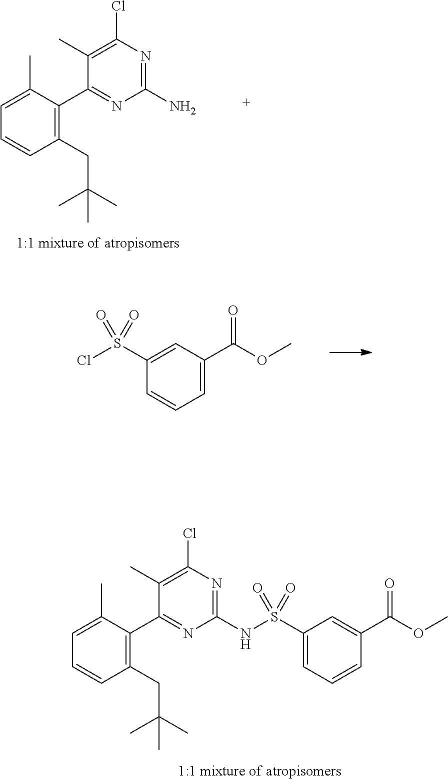 [SMILES: Cc1cccc(CC(C)(C)C)c1-c1nc(N)nc(Cl)c1C ; COC(=O)c1cccc(S(=O)(=O)Cl)c1 ; COC(=O)c1cccc(S(=O)(=O)Nc2nc(Cl)c(C)c(-c3c(C)cccc3CC(C)(C)C)n2)c1]

To a solution of 4-chloro-6-[2-(2,2-dimethylpropyl)-6-methyl-phenyl]-5-methyl-pyrimidin-2-amine (693 mg, 2.1782 mmol) and methyl 3-chlorosulfonylbenzoate (1.552 g, 6.6139 mmol) in anhydrous THF (12 mL) was added LiHMDS in THF (6.7 mL of 1.3 M, 8.71 mmol) dropwise at −78° C. The reaction was slowly warmed to rt and stirred for 2 h. The reaction was quenched with 10% aqueous citric acid (30 mL) and extracted with ethyl acetate (3×30 mL). The combined organic layers were washed with brine (30 mL), dried over anhydrous sodium sulfate and concentrated under vacuum. The residue was purified by silica gel chromatography using 0 to 20% acetone in hexane to furnish methyl 3-[[4-chloro-6-[2-(2,2-dimethylpropyl)-6-methyl-phenyl]-5-methyl-pyrimidin-2-yl]sulfamoyl]benzoate (1.084 g, 97%) as a white solid. ESI-MS m/z calc. 501.1489, found 502.1 (M+1)⁺; Retention time: 4.02 minutes; LC Method T.

### Step 6: 3-[[4-Chloro-6-[2-(2,2-dimethylpropyl)-6-methyl-phenyl]-5-methyl-pyrimidin-2-yl]sulfamoyl]benzoic acid

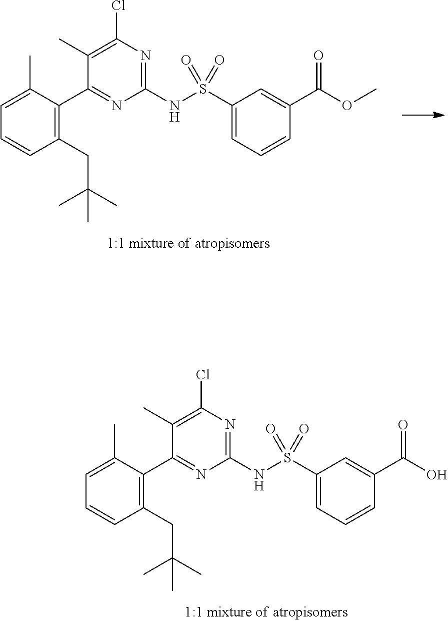 [SMILES: COC(=O)c1cccc(S(=O)(=O)Nc2nc(Cl)c(C)c(-c3c(C)cccc3CC(C)(C)C)n2)c1 ; Cc1cccc(CC(C)(C)C)c1-c1nc(NS(=O)(=O)c2cccc(C(=O)O)c2)nc(Cl)c1C]

To a solution of methyl 3-[[4-chloro-6-[2-(2,2-dimethylpropyl)-6-methyl-phenyl]-5-methyl-pyrimidin-2-yl]sulfamoyl]benzoate (1.084 g, 2.1161 mmol) in THF (11 mL) was added 1 N NaOH solution (11 mL, 11.0 mmol). The reaction was stirred at rt for 2 h. The reaction was diluted with 1 N HCl solution (30 mL) and TBME (30 mL). The layers were separated and the aqueous layer was extracted with TBME (2×30 mL). The combined organic layers were washed with brine (30 mL), dried over anhydrous magnesium sulfate and concentrated under vacuum to furnish 3-[[4-chloro-6-[2-(2,2-dimethylpropyl)-6-methyl-phenyl]-5-methyl-pyrimidin-2-yl]sulfamoyl]benzoic acid (1.076 g, 102%) as a white solid. ESI-MS m/z calc. 487.13324, found 488.1 (M+1)⁺; Retention time: 3.59 minutes; LC Method T.

### Step 7: 3-[[4-[(2R)-2-Amino-4,4-dimethyl-pentoxy]-6-[2-(2,2-dimethylpropyl)-6-methyl-phenyl]-5-methyl-pyrimidin-2-yl]sulfamoyl]benzoic acid

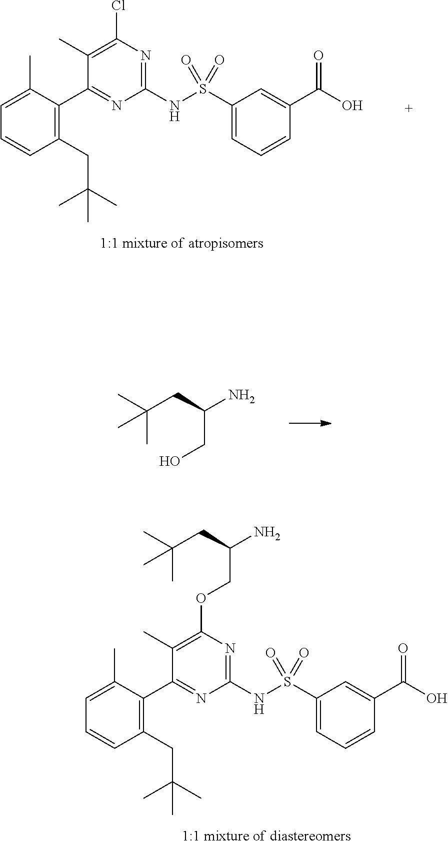

To a solution of 3-[[4-chloro-6-[2-(2,2-dimethylpropyl)-6-methyl-phenyl]-5-methyl-pyrimidin-2-yl]sulfamoyl]benzoic acid (1.076 g, 2.1608 mmol) and (2R)-2-amino-4,4-dimethyl-pentan-1-ol (hydrochloride salt) (561 mg, 3.3457 mmol) in anhydrous THF (20 mL) was added sodium tert-butoxide (1.047 g, 10.895 mmol). The reaction was stirred at rt for 2 h. Another portion of (2R)-2-amino-4,4-dimethyl-pentan-1-ol (hydrochloride salt) (147 mg, 0.8767 mmol) and sodium tert-butoxide (203 mg, 2.1123 mmol) was added. The reaction was stirred for 1 h, before it was quenched with 1 N HCl solution (30 mL). The reaction was extracted with ethyl acetate (3×30 mL). The combined organic layers were washed with brine (30 mL), dried over anhydrous magnesium sulfate and concentrated under vacuum. The residue was triturated with diethyl ether (10 mL) to furnish 3-[[4-[(2R)-2-amino-4,4-dimethyl-pentoxy]-6-[2-(2,2-dimethylpropyl)-6-methyl-phenyl]-5-methyl-pyrimidin-2-yl]sulfamoyl]benzoic acid (hydrochloride salt) (1.1193 g, 80%) as a white solid. ¹H NMR (500 MHz, DMSO-d₆) δ 8.44-8.38 (m, 1H), 8.16 (s, 3H), 8.09 (m, 2H), 7.66 (t, J=7.8 Hz, 1H), 7.35-7.23 (m, 1H), 7.23-7.08 (m, 2H), 4.35-4.21 (m, 1H), 4.17-4.05 (m, 1H), 3.65-3.52 (m, 1H), 2.39-2.26 (m, 1H), 2.15-2.02 (m, 1H), 2.02-1.81 (m, 3H), 1.72-1.63 (m, 3H), 1.62-1.48 (m, 2H), 0.95-0.88 (m, 9H), 0.79-0.57 (m, 9H). ESI-MS m/z calc. 582.2876, found 583.3 (M+1)⁺; Retention time: 2.19 minutes; LC Method W.

### Step 8: 5-(3,3-Dimethylpyrrolidin-1-yl)pyrimidine-2-carbonitrile

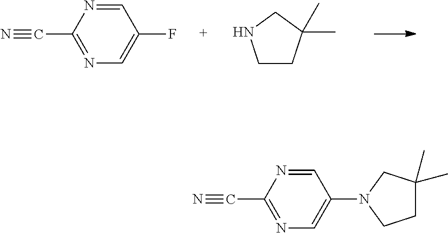

To a stirred solution of 5-fluoropyrimidine-2-carbonitrile (10 g, 77.181 mmol) and 3,3-dimethylpyrrolidine (hydrochloride salt) (13 g, 95.846 mmol) in DMF (100 mL) was added cesium carbonate (63 g, 193.36 mmol) at rt and stirred for 6 h. Water (400 mL) was added and the resulting precipitate was filtered. The solid cake was washed with water and dissolved in DCM (500 mL). The DCM layer was washed with brine, dried over sodium sulfate, filtered and concentrated in vacuo. The resulting beige solid was triturated with hexanes and filtered. The solid was washed with hexanes and dried in vacuo to give 5-(3,3-dimethylpyrrolidin-1-yl)pyrimidine-2-carbonitrile (15.1 g, 92%) as a white solid. ESI-MS m/z calc. 202.12184, found 203.5 (M+1)⁺; Retention time: 4.67 minutes; LC Method S.

### Step 9: 5-(3,3-Dimethylpyrrolidin-1-yl)pyrimidine-2-carbaldehyde

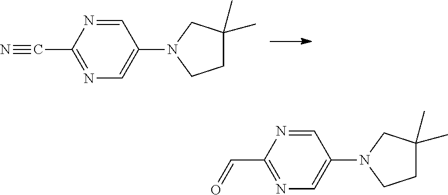

To a solution of 5-(3,3-dimethylpyrrolidin-1-yl)pyrimidine-2-carbonitrile (14.7 g, 69.046 mmol) in THF (300 mL) was added DIBAL in toluene (85 mL of 1 M, 85.0 mmol) at −78° C. dropwise over 30 min and stirred for 2 h. The reaction mixture was quenched with 500 mL of sat. aq. sodium potassium tartrate solution (Rochelle's salt) and stirred for 30 min while allowing to warm to rt. The layers were separated, and the aqueous layer was extracted with EtOAc (3×300 mL). The combined organic layer was washed with brine, dried over sodium sulfate, filtered and concentrated in vacuo. The resulting solid was purified by silica gel chromatography (eluted with 3% MeOH in DCM) to give 5-(3,3-dimethylpyrrolidin-1-yl)pyrimidine-2-carbaldehyde (8.08 g, 56%) as an orange solid. ¹H NMR (500 MHz, DMSO-d₆) δ 9.73 (s, 1H), 8.23 (s, 2H), 3.52 (t, J=7.0 Hz, 2H), 3.21 (s, 2H), 1.80 (t, J=7.0 Hz, 2H), 1.10 (s, 6H). ESI-MS m/z calc. 205.1215, found 206.2 (M+1)⁺; Retention time: 1.69 minutes; LC Method W.

### Step 10: (5M,11R)-11-(2,2-dimethylpropyl)-6-[2-(2,2-dimethylpropyl)-6-methylphenyl]-12-{[5-(3,3-dimethylpyrrolidin-1-yl)pyrimidin-2-yl]methyl}-7-methyl-9-oxa-2λ⁶-thia-3,5,12,19-tetraazatricyclo[12.3.1.14,8]nonadeca-1(17),4(19),5,7,14(18),15-hexaene-2,2,13-trione (Compound 214) and (5P,11R)-11-(2,2-dimethylpropyl)-6-[2-(2,2-dimethylpropyl)-6-methylphenyl]-12-{[5-(3,3-dimethylpyrrolidin-1-yl)pyrimidin-2-yl]methyl}-7-methyl-9-oxa-2λ⁶-thia-3,5,12,19-tetraazatricyclo[12.3.1.14,8]nonadeca-1(17),4(19),5,7,14(18),15-hexaene-2,2,13-trione (Compound 215)

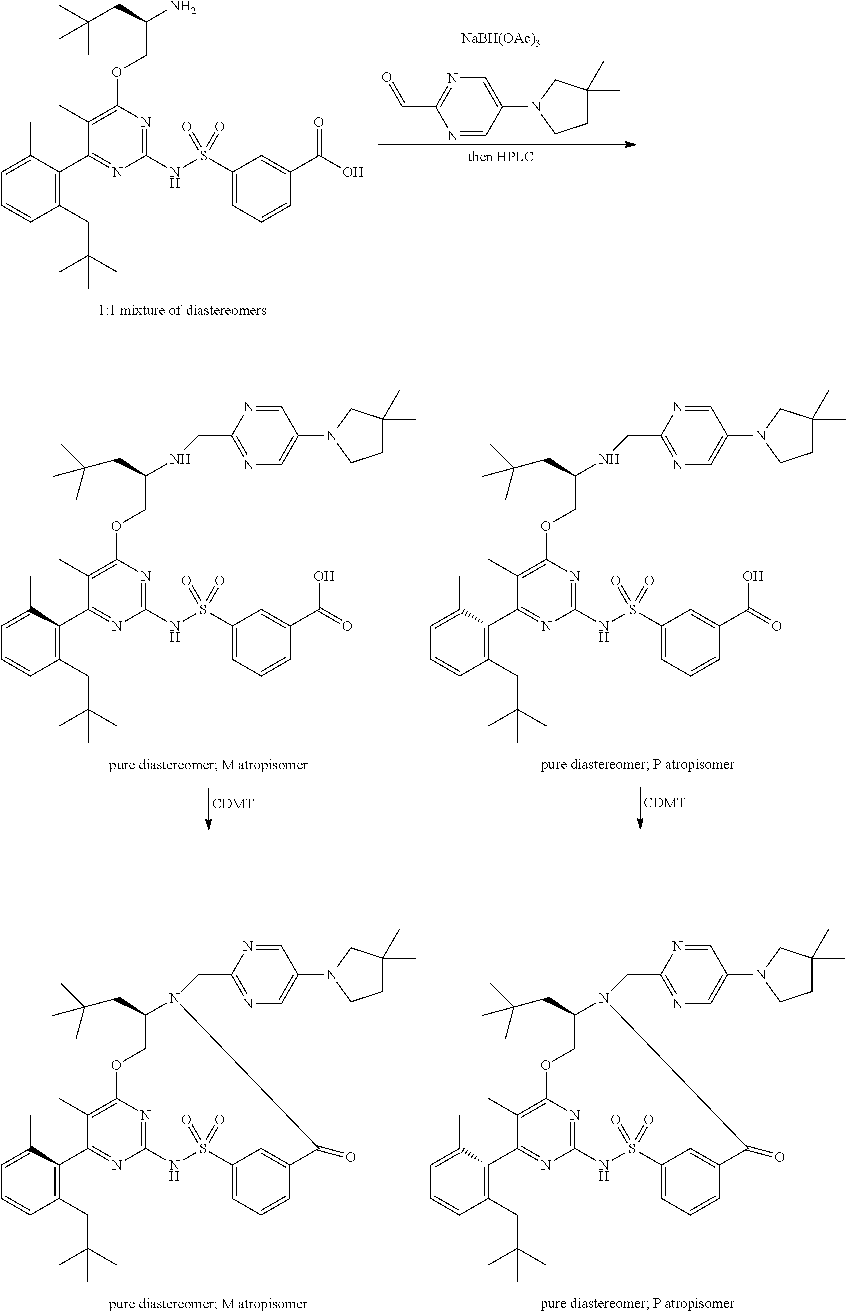

Stage 1: A 4-mL vial was charged under nitrogen with 3-[[4-[(2R)-2-amino-4,4-dimethyl-pentoxy]-6-[2-(2,2-dimethylpropyl)-6-methyl-phenyl]-5-methyl-pyrimidin-2-yl]sulfamoyl]benzoic acid hydrochloride (100 mg, 0.1615 mmol), 5-(3,3-dimethylpyrrolidin-1-yl)pyrimidine-2-carbaldehyde (38 mg, 0.1851 mmol), and DCM (450 μL). The mixture was stirred at room temperature for 10 min. Sodium triacetoxyborohydride (34 mg, 0.1604 mmol) was added and the mixture was stirred at room temperature for 20 min. More sodium triacetoxyborohydride (100 mg, 0.4718 mmol) was added and the mixture was stirred at room temperature for 3 h. The reaction was quenched with a minimum amount of 1 N HCl solution. Methanol and DMSO were added. After filtration, purification by reverse-phase HPLC (1-99% acetonitrile/5 mM HCl) gave Peak 1, 3-[[4-[2-(2,2-dimethylpropyl)-6-methyl-phenyl]-6-[(2R)-2-[[5-(3,3-dimethylpyrrolidin-1-yl)pyrimidin-2-yl]methylamino]-4,4-dimethyl-pentoxy]-5-methyl-pyrimidin-2-yl]sulfamoyl]benzoic acid (hydrochloride salt) (19.5 mg, 15%); ESI-MS m/z calc. 771.4142, found 772.7 (M+1)⁺; Retention time: 0.66 minutes; LC Method D. Peak 2 was also obtained: 3-[[4-[2-(2,2-dimethylpropyl)-6-methyl-phenyl]-6-[(2R)-2-[[5-(3,3-dimethylpyrrolidin-1-yl)pyrimidin-2-yl]methylamino]-4,4-dimethyl-pentoxy]-5-methyl-pyrimidin-2-yl]sulfamoyl]benzoic acid (hydrochloride salt) (18 mg, 14%) ESI-MS m/z calc. 771.4142, found 772.8 (M+1)⁺; Retention time: 0.69 minutes; LC Method D.

Stage 2 (from Peak 1): 3-[[4-[2-(2,2-dimethylpropyl)-6-methyl-phenyl]-6-[(2R)-2-[[5-(3,3-dimethylpyrrolidin-1-yl)pyrimidin-2-yl]methylamino]-4,4-dimethyl-pentoxy]-5-methyl-pyrimidin-2-yl]sulfamoyl]benzoic acid (hydrochloride salt) (19.5 mg, 15%) was combined in a 4-mL vial under with CDMT (7 mg, 0.03987 mmol) and DMF (2 mL). The solution was stirred at 0° C. 4-Methylmorpholine (25 μL, 0.2274 mmol) was added and the mixture was stirred in the cooling bath and allowed to warm to room temperature over 16 h. The reaction was filtered and purified by reverse phase HPLC (1-99% acetonitrile/5 mM HCl) to give the more polar, first-to-elute isomer: (5M,11R)-11-(2,2-dimethylpropyl)-6-[2-(2,2-dimethylpropyl)-6-methylphenyl]-12-{[5-(3,3-dimethylpyrrolidin-1-yl)pyrimidin-2-yl]methyl}-7-methyl-9-oxa-2λ⁶-thia-3,5,12,19-tetraazatricyclo[12.3.1.14,8]nonadeca-1(17),4(19),5,7,14(18),15-hexaene-2,2,13-trione (4.5 mg, 7%). ¹H NMR (400 MHz, DMSO-d₆) δ 12.98 (s, 1H), 8.69 (s, 1H), 8.07 (d, J=2.0 Hz, 2H), 7.88 (s, 1H), 7.64 (s, 2H), 7.32 (s, 1H), 7.21 (d, J=7.5 Hz, 1H), 7.14 (d, J=7.7 Hz, 1H), 5.52-5.28 (m, 1H), 4.87 (dd, J=16.2, 2.0 Hz, 1H), 4.54 (d, J=16.2 Hz, 1H), 4.11 (t, J=11.1 Hz, 1H), 4.03-3.94 (m, 1H), 3.41-3.39 (m, 2H), 3.08 (d, J=2.0 Hz, 2H), 2.26 (d, J=13.4 Hz, 1H), 2.07 (s, 3H), 1.96 (d, J=13.4 Hz, 1H), 1.87 (dd, J=15.7, 9.9 Hz, 1H), 1.77 (td, J=7.0, 2.0 Hz, 2H), 1.57 (s, 3H), 1.34 (d, J=15.1 Hz, 1H), 1.10 (d, J=2.0 Hz, 6H), 0.77-0.55 (m, 18H). ESI-MS m/z calc. 753.4036, found 754.7 (M+1)⁺; Retention time: 2.24 minutes; LC Method A.

Stage 2 (from Peak 2): 3-[[4-[2-(2,2-dimethylpropyl)-6-methyl-phenyl]-6-[(2R)-2-[[5-(3,3-dimethylpyrrolidin-1-yl)pyrimidin-2-yl]methylamino]-4,4-dimethyl-pentoxy]-5-methyl-pyrimidin-2-yl]sulfamoyl]benzoic acid (hydrochloride salt) (18 mg, 14%) was combined in a 4-mL vial with CDMT (7 mg, 0.03987 mmol) and DMF (2 mL). The solution was stirred at 0° C. 4-methylmorpholine (25 μL, 0.2274 mmol) was added, and the mixture was stirred in the cooling bath and allowed to warm to room temperature over 16 h. The reaction mixture was filtered and purified by reverse-phase HPLC (1-99% acetonitrile/5 mM HCl over 30 min) to give the less polar, second-to-elute isomer: (5P,11R)-11-(2,2-dimethylpropyl)-6-[2-(2,2-dimethylpropyl)-6-methylphenyl]-12-{[5-(3,3-dimethylpyrrolidin-1-yl)pyrimidin-2-yl]methyl}-7-methyl-9-oxa-2λ⁶-thia-3,5,12,19-tetraazatricyclo[12.3.1.14,8]nonadeca-1(17),4(19),5,7,14(18),15-hexaene-2,2,13-trione (6.9 mg, 11%). ¹H NMR (400 MHz, DMSO-d₆) δ 12.81 (s, 1H), 8.69 (s, 1H), 8.09 (s, 2H), 7.92 (d, J=7.5 Hz, 1H), 7.66 (dd, J=14.3, 6.7 Hz, 2H), 7.31 (t, J=7.7 Hz, 1H), 7.16 (t, J=8.2 Hz, 2H), 5.43 (dd, J=10.9, 4.1 Hz, 1H), 4.88 (d, J=16.1 Hz, 1H), 4.54 (d, J=16.3 Hz, 1H), 4.14 (t, J=11.1 Hz, 1H), 4.01 (d, J=11.3 Hz, 1H), 3.38 (d, J=7.1 Hz, 2H), 3.09 (s, 2H), 2.41 (d, J=13.2 Hz, 1H), 2.23 (d, J=13.3 Hz, 1H), 1.82 (d, J=6.2 Hz, 1H), 1.78 (d, J=5.6 Hz, 4H), 1.57 (s, 3H), 1.36 (d, J=15.0 Hz, 1H), 1.24 (d, J=7.7 Hz, 1H), 1.10 (s, 6H), 0.81 (s, 9H), 0.54 (s, 9H). ESI-MS m/z calc. 753.4036, found 754.7 (M+1)⁺; Retention time: 2.29 minutes; LC Method A.

### Example 75: Preparation of Compound 216

### Step 1: 2-Cyclopropyl-6-methyl-phenol

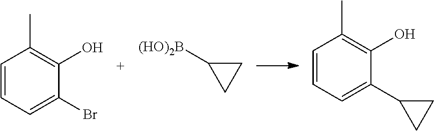

Palladium acetate (2.1 g, 9.3538 mmol) was added to 2-bromo-6-methyl-phenol (20 g, 106.93 mmol), cyclopropylboronic acid (13.8 g, 160.66 mmol), tricyclohexylphosphine (5 g, 17.830 mmol) and potassium phosphate (64.5 g, 303.86 mmol) in a suspension of toluene (640 mL) and water (160 mL). The reaction solution was refluxed for 6 h under nitrogen atmosphere. The reaction solution was cooled to room temperature, the organic phase was separated then washed with water (100 mL). The organic fraction was combined with heptane (˜300 mL) then filtered on a large pad of silica gel, then the silica gel was washed with a 1:1 heptane:DCM mixture. The fractions containing the desired product were combined and concentrated carefully (avoiding loss of the volatile desired product) to provide 2-cyclopropyl-6-methyl-phenol (17.7 g, 78%) as a brown oil. ¹H NMR (400 MHz, Chloroform-d) δ 7.02 (d, J=7.6 Hz, 1H), 6.97 (d, J=7.6 Hz, 1H), 6.78 (t, J=7.6 Hz, 1H), 5.56 (s, 1H), 2.28 (s, 3H), 1.79 (tt, J=8.3, 5.3 Hz, 1H), 1.04-0.94 (m, 2H), 0.69-0.62 (m, 2H).

### Step 2: (2-Cyclopropyl-6-methyl-phenyl) trifluoromethanesulfonate

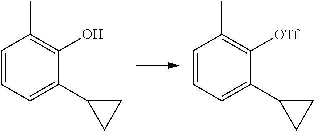 [SMILES: Cc1cccc(C2CC2)c1O ; Cc1cccc(C2CC2)c1OS(=O)(=O)C(F)(F)F]

Pyridine (15.648 g, 16 mL, 197.83 mmol) was added to a solution of 2-cyclopropyl-6-methyl-phenol (17.7 g, 119.43 mmol) in DCM (240 mL). The reaction mixture was cooled to 0° C., then trifluoromethylsulfonyl trifluoromethanesulfonate (43.602 g, 26 mL, 154.54 mmol) was slowly added. The reaction mixture was left to warm slowly to room temperature overnight. The reaction was diluted with DCM (200 mL) and the resulting mixture was washed with 0.2 N HCl (2×200 mL). The organic layer was washed with diluted NaHCO₃(100 mL, 10× diluted saturated NaHCO₃ solution), then dried with sodium sulfate, filtered and concentrated in vacuo to provide (2-cyclopropyl-6-methyl-phenyl) trifluoromethanesulfonate (22.13 g, 66%) as a yellow oil. ¹H NMR (400 MHz, Chloroform-d) δ 7.17 (t, J=8.6 Hz, 1H), 7.10 (d, J=6.8 Hz, 1H), 6.86 (d, J=7.3 Hz, 1H), 2.40 (s, 3H), 2.18-2.07 (m, 1H), 1.11-1.02 (m, 2H), 0.77-0.68 (m, 2H). ¹⁹F NMR (377 MHz, Chloroform-d) 6-73.43 (s, 3F).

### Step 3: 2-(2-Cyclopropyl-6-methyl-phenyl)-4,4,5,5-tetramethyl-1,3,2-dioxaborolane

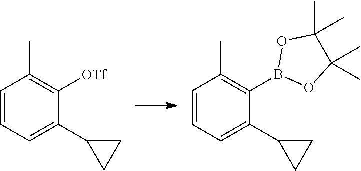 [SMILES: Cc1cccc(C2CC2)c1OS(=O)(=O)C(F)(F)F ; Cc1cccc(C2CC2)c1B1OC(C)(C)C(C)(C)O1]

A 500-mL round-bottomed flask was charged with molecular sieves 4 Å (7 g) and flame dried under vacuum. (2-Cyclopropyl-6-methyl-phenyl) trifluoromethanesulfonate (22 g, 78.498 mmol) then [1,1′-bis(diphenylphosphino)ferrocene]dichloropalladium(II) (2.8 g, 3.8267 mmol) were added. The flask was evacuated with nitrogen and then anhydrous dioxane (40 mL), triethyl amine (27.588 g, 38 mL, 272.64 mmol) and 4,4,5,5-tetramethyl-1,3,2-dioxaborolane (35.280 g, 40 mL, 275.67 mmol) were added. The reaction was refluxed for 4 h. The reaction mixture was cooled to room temperature, concentrated under reduced pressure, diluted with heptane and DCM, then purified by silica gel chromatography (with a gradient of 0 to 30% DCM in heptane) to provide 2-(2-cyclopropyl-6-methyl-phenyl)-4,4,5,5-tetramethyl-1,3,2-dioxaborolane (12.4 g, 60%) as a white solid. ¹H NMR (400 MHz, Chloroform-d) δ 7.13 (t, J=7.6 Hz, 1H), 6.94 (d, J=7.6 Hz, 1H), 6.74 (d, J=7.8 Hz, 1H), 2.40 (s, 3H), 2.11 (tt, J=8.5, 5.2 Hz, 1H), 1.41 (s, 12H), 0.93-0.81 (m, 2H), 0.74-0.64 (m, 2H). ESI-MS m/z calc. 258.1791, found 259.2 (M+1)⁺; Retention time: 4.98 minutes; LC Method Y.

### Step 4: tert-Butyl N-tert-butoxycarbonyl-N-[4-chloro-6-(2-cyclopropyl-6-methyl-phenyl)-5-methyl-pyrimidin-2-yl]carbamate

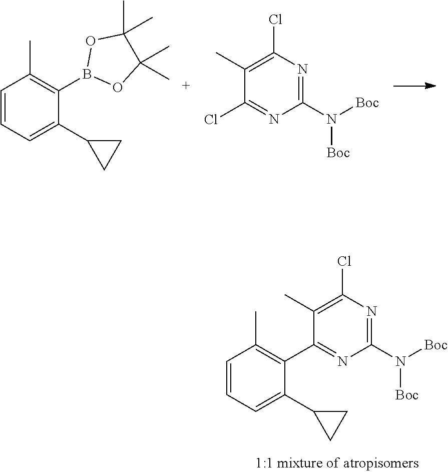 [SMILES: Cc1cccc(C2CC2)c1B1OC(C)(C)C(C)(C)O1 ; Cc1c(Cl)nc(N(C(=O)OC(C)(C)C)C(=O)OC(C)(C)C)nc1Cl ; Cc1cccc(C2CC2)c1-c1nc(N(C(=O)OC(C)(C)C)C(=O)OC(C)(C)C)nc(Cl)c1C]

A mixture of 2-(2-cyclopropyl-6-methyl-phenyl)-4,4,5,5-tetramethyl-1,3,2-dioxaborolane (1.50 g, 5.7231 mmol), tert-butyl N-tert-butoxycarbonyl-N-(4,6-dichloro-5-methyl-pyrimidin-2-yl)carbamate (2.03 g, 5.3668 mmol), and cesium carbonate (4.73 g, 14.517 mmol) in DME (30 mL) and water (30 mL) was purged with nitrogen for 15 min. Then, Pd(dppf)Cl₂ (422.7 mg, 0.5777 mmol) was added and the mixture was further purged with nitrogen for 5 min before stirring at 80° C. for 2 h. Upon cooling to room temperature, H₂O (30 mL) and EtOAc (30 mL) were added. The layers were separated and the aqueous layer was extracted with EtOAc (2×30 mL). The combined EtOAc layers were washed with saturated aqueous NaCl (30 mL), dried over anhydrous sodium sulfate, filtered and concentrated in vacuo. The crude product was subjected to silica gel chromatography (80 g SiO₂, eluting with 0 to 10% EtOAc/hexanes) to give a colorless oil, tert-butyl N-tert-butoxycarbonyl-N-[4-chloro-6-(2-cyclopropyl-6-methyl-phenyl)-5-methyl-pyrimidin-2-yl]carbamate (1.45 g, 51%); ESI-MS m/z calc. 473.20813, found 474.1 (M+1)⁺; Retention time: 7.71 minutes; LC Method S.

### Step 5: 4-Chloro-6-(2-cyclopropyl-6-methyl-phenyl)-5-methyl-pyrimidin-2-amine

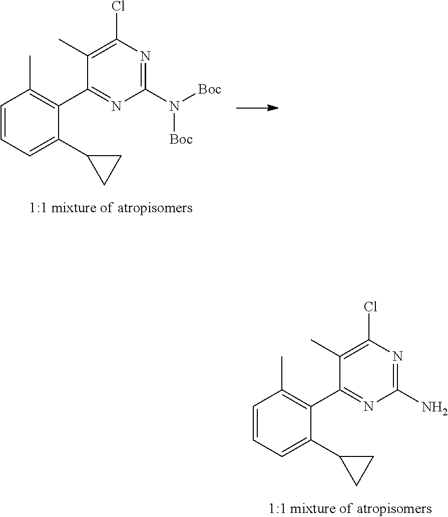 [SMILES: Cc1cccc(C2CC2)c1-c1nc(N(C(=O)OC(C)(C)C)C(=O)OC(C)(C)C)nc(Cl)c1C ; Cc1cccc(C2CC2)c1-c1nc(N)nc(Cl)c1C]

To a solution of tert-butyl N-tert-butoxycarbonyl-N-[4-chloro-6-(2-cyclopropyl-6-methyl-phenyl)-5-methyl-pyrimidin-2-yl]carbamate (1.45 g, 2.9062 mmol) in DCM (13 mL) stirring at 0° C. was added HCl in dioxane (20 mL of 4 M, 80.0 mmol). The reaction was then stirred at RT for 16 h. The reaction mixture was diluted with DCM (25 mL) and neutralized with saturated aqueous sodium bicarbonate (40 mL). The layers were separated and the aqueous layer was further extracted with DCM (2×25 mL). The combined DCM layers were washed with saturated aqueous NaCl (25 mL), dried over anhydrous sodium sulfate, filtered and concentrated under vacuum to give a white solid, 4-chloro-6-(2-cyclopropyl-6-methyl-phenyl)-5-methyl-pyrimidin-2-amine (952.4 mg, 109%); ¹H NMR (500 MHz, DMSO-d₆) δ 7.21 (t, J=7.7 Hz, 1H), 7.08 (d, J=7.5 Hz, 1H), 6.96 (s, 2H), 6.77 (d, J=7.8 Hz, 1H), 1.96 (s, 3H), 1.82 (s, 3H), 1.47-1.35 (m, 1H), 0.84-0.72 (m, 2H), 0.72-0.62 (m, 1H), 0.56-0.43 (m, 1H). ESI-MS m/z calc. 273.10327, found 274.0 (M+1)⁺; Retention time: 2.33 minutes; LC Method T.

### Step 6: (5P)-4-Chloro-6-(2-cyclopropyl-6-methylphenyl)-5-methylpyrimidin-2-amine and (5M)-4-chloro-6-(2-cyclopropyl-6-methylphenyl)-5-methylpyrimidin-2-amine

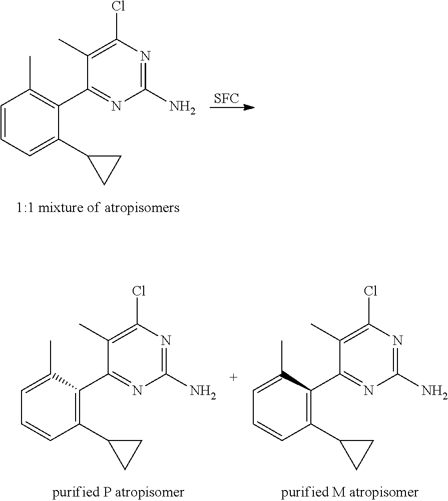

Racemic 4-chloro-6-(2-cyclopropyl-6-methyl-phenyl)-5-methyl-pyrimidin-2-amine (920 mg, 3.361 mmol) was subjected to chiral SFC separation using the following conditions: AD-H column (2×15 cm), eluant: 20% MeOH/CO₂, flow rate: 70 mL/min, concentration: 20 mg/mL in methanol:DCM, injection volume: 500 μL, pressure: 100 bar, wavelength: 220 nm. Two isomers were isolated. SFC peak 1: (5P)-4-chloro-6-(2-cyclopropyl-6-methylphenyl)-5-methylpyrimidin-2-amine (357 mg, 74%). ¹H NMR (400 MHz, DMSO-d₆) δ 7.20 (t, J=7.7 Hz, 1H), 7.08 (dt, J=7.6, 1.0 Hz, 1H), 6.90 (broad s, 2H), 6.77 (d, J=7.8 Hz, 1H), 1.96 (s, 3H), 1.82 (s, 3H), 1.47-1.31 (m, 1H), 0.81-0.71 (m, 2H), 0.70-0.63 (m, 1H), 0.53-0.46 (m, 1H). ESI-MS m/z calc. 273.10327, found 274.17 (M+1)⁺; Retention time: 1.59 minutes; LC Method A. SFC peak 2: (5M)-4-chloro-6-(2-cyclopropyl-6-methylphenyl)-5-methylpyrimidin-2-amine (344 mg, 73%). ¹H NMR (400 MHz, DMSO-d₆) δ 7.20 (t, J=7.7 Hz, 1H), 7.08 (dt, J=7.5, 1.0 Hz, 1H), 6.89 (broad s, 2H), 6.77 (d, J=7.8 Hz, 1H), 1.96 (s, 3H), 1.82 (s, 3H), 1.47-1.34 (m, 1H), 0.80-0.71 (m, 2H), 0.71-0.64 (m, 1H), 0.53-0.46 (m, 1H). ESI-MS m/z calc. 273.10327, found 274.17 (M+1)⁺; Retention time: 1.59 minutes; LC Method A.

### Step 7: (5P)-Methyl 3-[[4-chloro-6-(2-cyclopropyl-6-methyl-phenyl)-5-methyl-pyrimidin-2-yl]sulfamoyl]benzoate

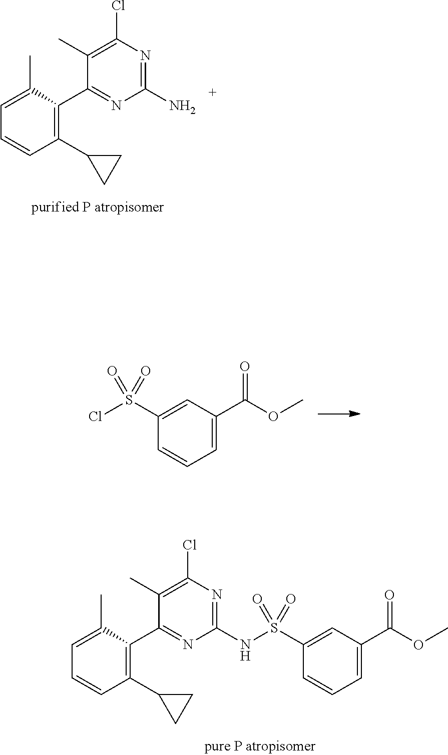

To a solution of (5P)-4-chloro-6-(2-cyclopropyl-6-methyl-phenyl)-5-methyl-pyrimidin-2-amine (300 mg, 1.0958 mmol) and methyl 3-chlorosulfonylbenzoate (770 mg, 3.2814 mmol) in THF (6.0 mL) was added a solution of LiHMDS in THF (4.2 mL of 1.0 M, 4.20 mmol) at −78° C. The reaction mixture was stirred for 2 h and then quenched with 1 N HCl solution (5 mL). The reaction mixture was warmed to room temperature, diluted with water (20 mL) and extracted with EtOAc (3×25 mL). The combined organic layers were washed with brine, dried over sodium sulfate, filtered and concentrated in vacuo. The resulting yellow solid was purified by silica gel chromatography (40 g silica, eluted with 20% ethyl acetate in hexanes) to give (5P)-methyl 3-[[4-chloro-6-(2-cyclopropyl-6-methyl-phenyl)-5-methyl-pyrimidin-2-yl]sulfamoyl]benzoate (370 mg, 71%) as a white solid. ESI-MS m/z calc. 471.10196, found 472.4 (M+1)⁺; Retention time: 6.69 minutes; LC Method S.

### Step 8: (5P)-3-[[4-Chloro-6-(2-cyclopropyl-6-methyl-phenyl)-5-methyl-pyrimidin-2-yl]sulfamoyl]benzoic acid

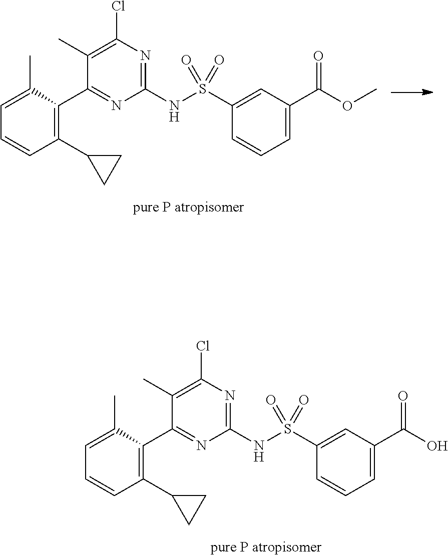

To a solution of (5P)-methyl 3-[[4-chloro-6-(2-cyclopropyl-6-methyl-phenyl)-5-methyl-pyrimidin-2-yl]sulfamoyl]benzoate (370 mg, 0.7761 mmol) in THF (7.4 mL) was added aqueous NaOH (4 mL of 1 M, 4.0 mmol) and stirred at rt for 2 h. The reaction mixture was diluted with water (25 mL) and extracted with ethyl acetate (50 mL). The aqueous layer was acidified to pH=1 and then extracted with ethyl acetate (50 mL). The organic layer was washed with brine, dried over sodium sulfate, filtered and concentrated in vacuo to give (5P)-3-[[4-chloro-6-(2-cyclopropyl-6-methyl-phenyl)-5-methyl-pyrimidin-2-yl]sulfamoyl]benzoic acid (352 mg, 97%) as a white solid. ESI-MS m/z calc. 457.0863, found 458.4 (M+1)⁺; Retention time: 6.38 minutes; LC Method S.

### Step 9: (5P)-3-[[4-[(2R)-2-Amino-4,4-dimethyl-pentoxy]-6-(2-cyclopropyl-6-methyl-phenyl)-5-methyl-pyrimidin-2-yl]sulfamoyl]benzoic acid

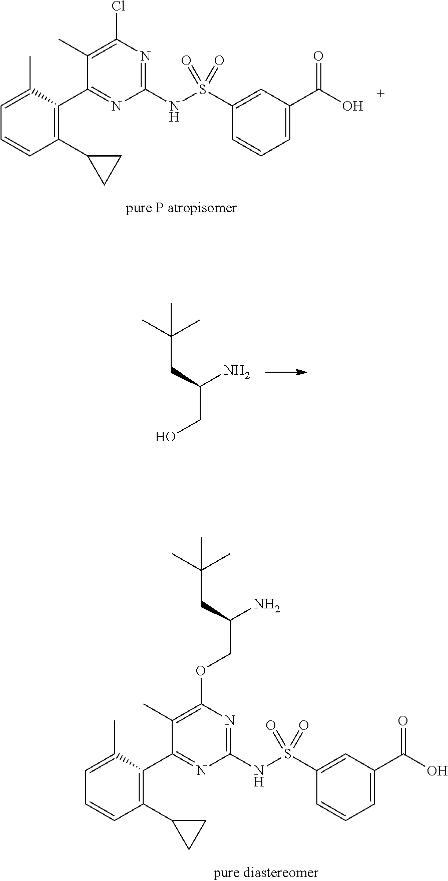

To a stirred solution of (5P)-3-[[4-chloro-6-(2-cyclopropyl-6-methyl-phenyl)-5-methyl-pyrimidin-2-yl]sulfamoyl]benzoic acid (350 mg, 0.7490 mmol) and (2R)-2-amino-4,4-dimethyl-pentan-1-ol (hydrochloride salt) (300 mg, 1.7892 mmol) in THF (7.0 mL) was added sodium t-butoxide (350 mg, 3.6419 mmol) at rt and stirred for 4 h. Another portion of (2R)-2-amino-4,4-dimethyl-pentan-1-ol (hydrochloride salt) (150 mg, 0.8946 mmol) and sodium t-butoxide (150 mg, 1.5608 mmol) was added and the reaction mixture was stirred for 1 h. The reaction mixture was cooled to 0° C. and quenched with 10 mL of 1 N HCl solution. The layers were separated and the aqueous layer was extracted with ethyl acetate (2×25 mL). The combined organic layers were washed with brine, dried over sodium sulfate, filtered and concentrated in vacuo. The resulting white solid was purified by reverse-phase HPLC (C18 column, with a gradient of 30 to 75% acetonitrile in water, with 0.1% HCl as a modifier) to give (5P)-3-[[4-[(2R)-2-amino-4,4-dimethyl-pentoxy]-6-(2-cyclopropyl-6-methyl-phenyl)-5-methyl-pyrimidin-2-yl]sulfamoyl]benzoic acid (hydrochloride salt) (332 mg, 73%) as a white solid. ¹H NMR (500 MHz, DMSO-d₆) δ 13.33 (s, 1H), 8.44 (s, 1H), 8.10 (dd, J=13.4, 7.7 Hz, 2H), 7.99 (bs, 3H), 7.66 (dd, J=7.8, 7.8 Hz, 1H), 7.26 (dd, J=7.7, 7.7 Hz, 1H), 7.11 (d, J=7.6 Hz, 1H), 6.81 (d, J=7.9 Hz, 1H), 4.24 (dd, J=12.1, 2.9 Hz, 1H), 4.07 (dd, J=12.1, 6.3 Hz, 1H), 3.62-3.55 (m, 1H), 1.91 (bs, 3H), 1.70 (s, 3H), 1.62 (dd, J=14.6, 7.2 Hz, 1H), 1.48 (dd, J=14.6, 3.9 Hz, 1H), 1.38-1.27 (m, 1H), 0.92 (s, 9H), 0.76-0.61 (m, 3H), 0.60-0.48 (m, 1H). ESI-MS m/z calc. 552.24066, found 553.3 (M+1)⁺; Retention time: 1.94 minutes; LC Method W.

### Step 10: 5-Fluoropyrimidine-2-carbaldehyde

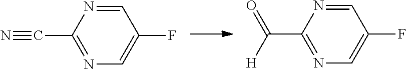 [SMILES: N#Cc1ncc(F)cn1 ; O=Cc1ncc(F)cn1]

To a solution of 5-fluoropyrimidine-2-carbonitrile (10 g, 77.993 mmol) in anhydrous THE (200 mL) was added 1.0 M DIBAL in toluene (117 mL of 1 M, 117.0 mmol) at −78° C. dropwise over 30 min. After the addition, the reaction was stirred for another 2 h at −78° C. Methanol (40 mL) was added to the reaction mixture at −78° C. The reaction temperature was slowly raised to rt, and then it was diluted with 10% aq. HCl (60 mL) and concentrated HCl (20 mL) (pH=3). Solid NaCl was added to saturate the aqueous layer. The reaction mixture was stirred for 1 h until both layers were clear. The layers were separated, and the aqueous layer was extracted with DCM (10×100 mL). The combined organic layers were dried over anhydrous sodium sulfate and concentrated under slight vacuum (>100 mbar, bath temp. 40° C.). The obtained product in toluene was directly loaded onto a silica gel column, and purified using 0 to 60% diethyl ether in DCM. The obtained fractions were combined and concentrated under slight vacuum (>100 mbar, bath temp. 40° C.) to furnish 5-fluoropyrimidine-2-carbaldehyde (5.545 g, 54%) as a yellow liquid. ¹H NMR (500 MHz, DMSO-d₆) δ 9.96 (s, 1H), 9.17 (d, J=0.8 Hz, 2H). ESI-MS m/z calc. 126.02294, found 127.2 (M+1)⁺; Retention time: 0.34 minutes; LC Method W.

### Step 11: 5-Morpholinopyrimidine-2-carbaldehyde

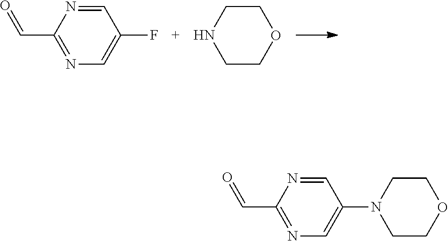 [SMILES: O=Cc1ncc(F)cn1 ; C1COCCN1 ; O=Cc1ncc(N2CCOCC2)cn1]

To a solution of 5-fluoropyrimidine-2-carbaldehyde (1.29 g, 6.6194 mmol) in anhydrous DMF (10 mL) were added morpholine (1.1988 g, 1.2 mL, 13.760 mmol) and potassium carbonate (3.65 g, 26.410 mmol). The reaction was stirred at 110° C. for 4 h. After centrifuging, the DMF solution was directly subjected to HPLC purification using 0 to 40% MeCN in water (buffered with 0.1% HCl) to furnish 5-morpholinopyrimidine-2-carbaldehyde (hydrochloride salt) (1.4515 g, 91%) as a yellow solid. ¹H NMR (500 MHz, DMSO-d₆) δ 9.79 (s, 1H), 8.65 (s, 2H), 3.82-3.66 (m, 4H), 3.54-3.37 (m, 4H). ESI-MS m/z calc. 193.08513, found 194.3 (M+1)⁺; Retention time: 1.27 minutes; LC Method W.

### Step 12: (5P)-3-[[4-(2-Cyclopropyl-6-methyl-phenyl)-6-[(2R)-4,4-dimethyl-2-[(5-morpholinopyrimidin-2-yl)methylamino]pentoxy]-5-methyl-pyrimidin-2-yl]sulfamoyl]benzoic acid

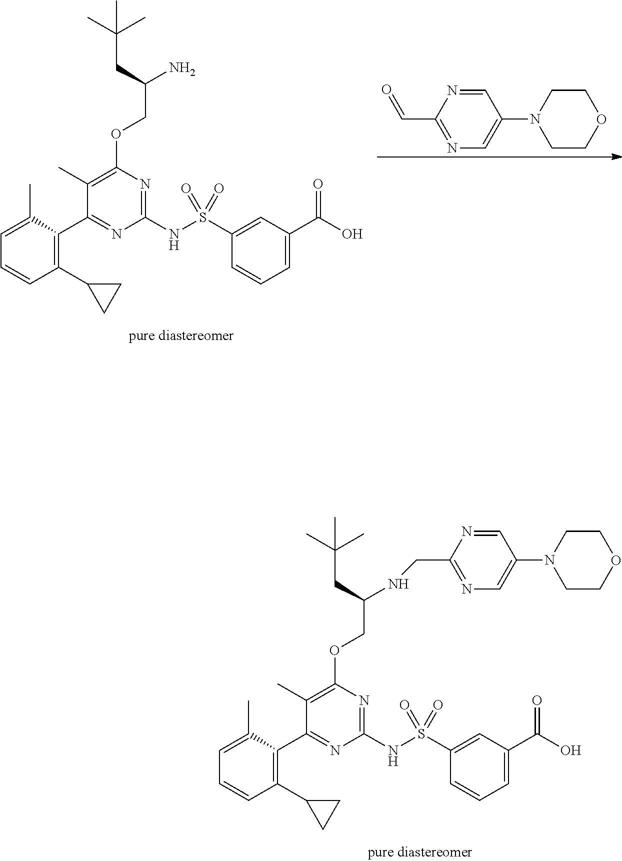

To a stirred mixture of (5P)-3-[[4-(2-amino-4,4-dimethyl-pentoxy)-6-(2-cyclopropyl-6-methyl-phenyl)-5-methyl-pyrimidin-2-yl]sulfamoyl]benzoic acid (hydrochloride salt) (100 mg, 0.1680 mmol) and 5-morpholinopyrimidine-2-carbaldehyde (hydrochloride salt) (50 mg, 0.2177 mmol) in DCM (1 mL) was added sodium triacetoxyborohydride (190 mg, 0.8965 mmol) at rt and stirred for 30 min. The reaction mixture was quenched with 1 N HCl solution (0.5 mL), diluted with DCM (10 mL) and filtered. The filtrate was concentrated in vacuo and the resulting red oil was purified by reverse-phase HPLC (C₁₈ column, with a gradient of 30 to 90% acetonitrile in water, with 0.1% HCl as a modifier) to give (5P)-3-[[4-(2-cyclopropyl-6-methyl-phenyl)-6-[4,4-dimethyl-2-[(5-morpholinopyrimidin-2-yl)methylamino]pentoxy]-5-methyl-pyrimidin-2-yl]sulfamoyl]benzoic acid (hydrochloride salt) (88 mg, 68%) as a pale yellow solid. ESI-MS m/z calc. 729.3309, found 731.0 (M+1)⁺; Retention time: 5.64 minutes; LC Method W.

### Step 13: (5P,11R)-6-(2-Cyclopropyl-6-methyl-phenyl)-11-(2,2-dimethylpropyl)-7-methyl-12-[(5-morpholinopyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 216)

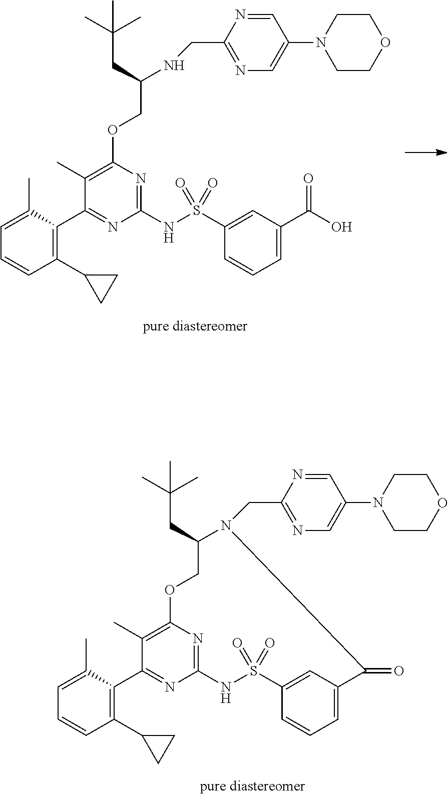

To a stirred solution of (5P)-3-[[4-(2-cyclopropyl-6-methyl-phenyl)-6-[(2R)-4,4-dimethyl-2-[(5-morpholinopyrimidin-2-yl)methylamino]pentoxy]-5-methyl-pyrimidin-2-yl]sulfamoyl]benzoic acid (90 mg, 0.1221 mmol) and CDMT (33 mg, 0.1880 mmol) in DMF (1 mL) was added NMM (64.400 mg, 70 μL, 0.6367 mmol) and reaction mixture was stirred at rt for 2 h. Then, another portion of CDMT (33 mg, 0.1880 mmol) and NMM (64.400 mg, 70 μL, 0.6367 mmol) was added and stirred at rt for 1 h. The reaction mixture was filtered and the filtrate was purified by HPLC (C18 column, with a gradient of 30 to 90% acetonitrile in water, with 0.1% HCl as a modifier) to give (5P,11R)-6-(2-cyclopropyl-6-methyl-phenyl)-11-(2,2-dimethylpropyl)-7-methyl-12-[(5-morpholinopyrimidin-2-yl)methyl]-2,2-dioxo-9-oxa-2⁶-thia-3,5,12,19-tetrazatricyclo[12.3.1.14,8]nonadeca-1(18),4(19),5,7,14,16-hexaen-13-one (22.6 mg, 25%) as a yellow powder. ¹H NMR (500 MHz, DMSO-d₆) δ 8.70 (s, 1H), 8.48 (s, 2H), 7.93 (d, J=7.6 Hz, 1H), 7.65 (d, J=16.7 Hz, 2H), 7.26 (dd, J=7.7, 7.7 Hz, 1H), 7.08 (d, J=7.6 Hz, 1H), 6.84 (d, J=7.9 Hz, 1H), 5.40 (d, J=8.5 Hz, 1H), 4.85 (d, J=16.4 Hz, 1H), 4.60 (d, J=16.4 Hz, 1H), 4.26-4.14 (m, 1H), 4.04 (m, J=9.9 Hz, 1H), 3.74 (dd, J=6.1, 3.7 Hz, 4H), 3.28-3.18 (m, 4H), 1.93-1.68 (m, 4H), 1.62 (s, 3H), 1.56-1.44 (m, 1H), 1.37 (d, J=15.0 Hz, 1H), 0.87-0.74 (m, 2H), 0.74-0.63 (m, 1H), 0.54 (s, 1OH). ESI-MS m/z calc. 711.3203, found 712.6 (M+1)⁺; Retention time: 2.57 minutes; LC Method W.

### Example 76: Preparation of Compound 217 and 218

### Step 1: 2-Bromo-1-(bromomethyl)-3-methyl-benzene

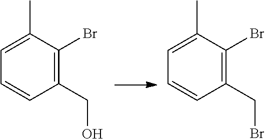

To a stirred solution of (2-bromo-3-methyl-phenyl)methanol (74.3 g, 369.54 mmol) in diethyl ether (750 mL) under nitrogen atmosphere at 0° C. was added phosphorus tribromide (120.26 g, 43.5 mL, 430.95 mmol). The reaction was warmed to room temperature then stirred overnight. Then, the reaction was cooled to 0° C., then methanol (400 mL) and water (600 mL) were added over 20 min to quench the reaction. The layers were separated, and the organic layer was extracted with Et₂O (2×100 mL). The combined organic layers were washed with brine (100 mL), dried over sodium sulfate, filtered and concentrated under reduced pressure to give 2-bromo-1-(bromomethyl)-3-methyl-benzene (96.5 g, 94%); ¹H NMR (400 MHz, CDCl₃) δ 7.29 (t, J=4.7 Hz, 1H), 7.21-7.16 (m, 2H), 4.65 (s, 2H), 2.44 (s, 3H).

### Step 2: (2-Bromo-3-methyl-phenyl)methyl-triphenyl-phosphonium bromide

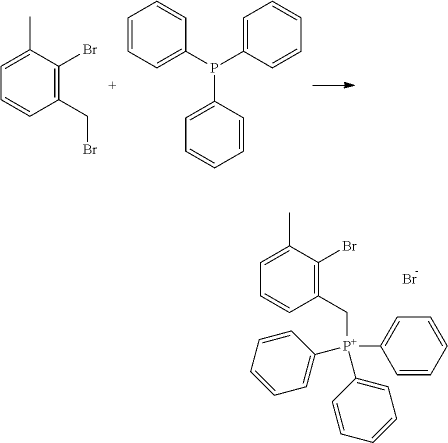

To a stirred solution of 2-bromo-1-(bromomethyl)-3-methyl-benzene (50 g, 189.42 mmol) in toluene (600 mL) was added triphenylphosphine (53 g, 200.05 mmol). The mixture was stirred at 60° C. for 12 h and then at room temperature for 64 h. The precipitate was filtered and washed with heptane (4×100 mL), then dried in vacuo to furnish (2-bromo-3-methyl-phenyl)methyl-triphenyl-phosphonium bromide (88 g, 84%) as a white solid. ¹H NMR (400 MHz, DMSO-d₆) δ 7.97-7.89 (m, 3H), 7.78-7.69 (m, 6H), 7.64-7.55 (m, 6H), 7.35 (br d, J=7.6 Hz, 1H), 7.20 (t, J=7.6 Hz, 1H), 7.04-6.95 (m, 1H), 5.18 (d, J=14.7 Hz, 2H), 2.21 (s, 3H). ¹H NMR (400 MHz, DMSO-d₆) δ 7.97-7.89 (m, 3H), 7.78-7.69 (m, 6H), 7.64-7.55 (m, 6H), 7.35 (br d, J=7.6 Hz, 1H), 7.20 (t, J=7.6 Hz, 1H), 7.04-6.95 (m, 1H), 5.18 (d, J=14.7 Hz, 2H), 2.21 (s, 3H).

### Step 3: 4-[(2-Bromo-3-methyl-phenyl)methylene]tetrahydropyran

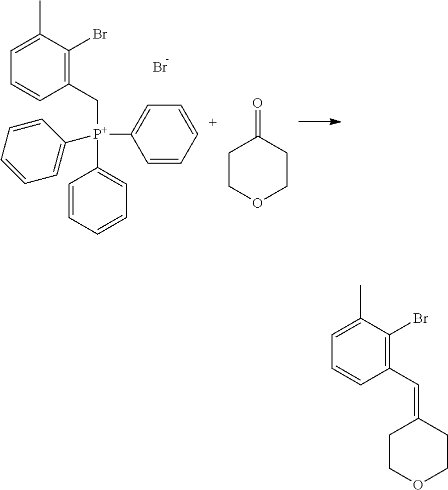 [SMILES: Cc1cccc(C[P+](c2ccccc2)(c2ccccc2)c2ccccc2)c1Br ; Br ; O=C1CCOCC1 ; Cc1cccc(C=C2CCOCC2)c1Br]

To a flask containing ethanol 150 mL at 0° C. under nitrogen atmosphere were added (2-bromo-3-methyl-phenyl)methyl-triphenyl-phosphonium bromide (11.5 g, 21.853 mmol) and a solution of sodium ethoxide in ethanol (8.1 mL of 2.67 M, 21.627 mmol). The solution was stirred at 0° C. for 5 min, after which tetrahydropyran-4-one (2.1546 g, 2.1 mL, 20.445 mmol) was added. The reaction was stirred at room temperature for 20 h then concentrated under reduced pressure. The crude was diluted in DCM (100 mL) and washed with water (100 mL). The phases were separated and the aqueous layer was extracted with DCM (3×50 mL). The combined organic layers were washed with aqueous saturated NH₄Cl (50 mL), brine (50 mL), dried over sodium sulfate, filtered and concentrated under reduced pressure. The crude mixture was triturated with MTBE (100 mL) and filtered. The filtrate was concentrated under reduced pressure. The crude was suspended in heptanes (30 mL) and was filtered over silica gel, eluting with a 1:1 mixture of MTBE/heptanes (150 mL). The volatiles were removed from the filtrate under reduced pressure. This mixture was purified by distillation under reduced pressure at 100-110° C. The distilling flask contained 4-[(2-bromo-3-methyl-phenyl)methylene]tetrahydropyran (3 g, 51%) as an orange oil. ¹H NMR (400 MHz, CDCl₃) δ 7.20-7.10 (m, 2H), 7.01 (dd, J=7.1, 2.2 Hz, 1H), 6.32 (s, 1H), 3.82 (t, J=5.4 Hz, 2H), 3.67 (t, J=5.5 Hz, 2H), 2.47-2.41 (m, 5H), 2.39-2.33 (m, 2H). ESI-MS m/z calc. 266.03064, found 267.2 (M+1)⁺; Retention time: 2.04 minutes; LC Method X.

### Step 4: [2-Methyl-6-(tetrahydropyran-4-ylidenemethyl)phenyl]boronic acid

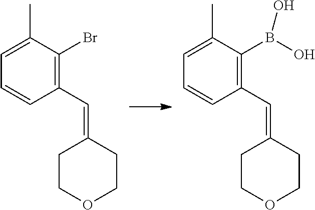 [SMILES: Cc1cccc(C=C2CCOCC2)c1Br ; Cc1cccc(C=C2CCOCC2)c1B(O)O]

To a tube was added XPhosPd-G4 catalyst (470 mg, 0.5462 mmol), XPhos (470 mg, 0.9859 mmol), tetrahydroxydiboron (3 g, 33.46 mmol) and potassium acetate (3 g, 30.568 mmol). A degassed solution of 4-[(2-bromo-3-methyl-phenyl)methylene]tetrahydropyran (3 g, 10.937 mmol) in ethanol (75 mL) was added and the tube was sealed. The reaction was stirred at 85° C. for 1 h. Then, the reaction mixture was cooled to room temperature. The crude mixture was filtered over Celite and the filter cake was washed with ethanol (100 mL). The volatiles were removed from the filtrate under reduced pressure. The crude was purified by reverse-phase chromatography (C18 column) using a gradient of MeCN in acidic water (0.1% v/v of formic acid in water) of 20 to 100%. The volatiles were removed from the fractions containing the compound. The resulting aqueous phase was extracted with DCM (10×30 mL). The combined organic layers were concentrated under reduced pressure to give [2-methyl-6-(tetrahydropyran-4-ylidenemethyl)phenyl]boronic acid (1.35 g, 53%) as a white solid. ¹H NMR (400 MHz, CDCl₃) δ 7.23 (t, J=7.8 Hz, 1H), 7.07 (d, J=7.6 Hz, 1H), 6.98 (d, J=7.6 Hz, 1H), 6.43 (s, 1H), 4.63-4.54 (m, 2H), 3.78 (t, J=5.4 Hz, 2H), 3.65 (t, J=5.5 Hz, 2H), 2.45 (s, 3H), 2.42-2.36 (m, 4H). ESI-MS m/z calc. 232.1271, found 231.2 (M−1)⁻; Retention time: 2.44 minutes; LC Method 1D.

### Step 5: tert-Butyl N-tert-butoxycarbonyl-N-[4-chloro-5-methyl-6-[2-methyl-6-(tetrahydropyran-4-ylidenemethyl)phenyl]pyrimidin-2-yl]carbamate

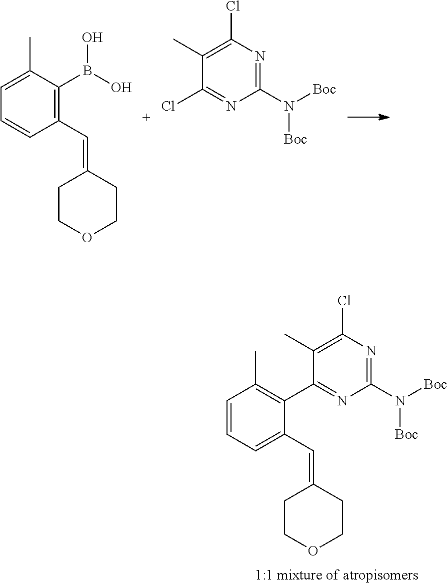 [SMILES: Cc1cccc(C=C2CCOCC2)c1B(O)O ; Cc1c(Cl)nc(N(C(=O)OC(C)(C)C)C(=O)OC(C)(C)C)nc1Cl ; Cc1cccc(C=C2CCOCC2)c1-c1nc(N(C(=O)OC(C)(C)C)C(=O)OC(C)(C)C)nc(Cl)c1C]

A reaction vial was charged with [2-methyl-6-(tetrahydropyran-4-ylidenemethyl)phenyl]boronic acid (528 mg, 2.2750 mmol), tert-butyl N-tert-butoxycarbonyl-N-(4,6-dichloro-5-methyl-pyrimidin-2-yl)carbamate (857 mg, 2.0844 mmol) and Cs₂CO₃ (2.048 g, 6.2857 mmol) in DME (9 mL) and water (3 mL). The reaction was purged with argon for 2 min. Pd(dppf)Cl₂ (77 mg, 0.1052 mmol) was added. The reaction was purged with argon for another 2 min. The vial was sealed and heated to 80° C., and stirred for 3 h. The reaction was diluted with ethyl acetate (40 mL), and washed with brine (20 mL). The organic layer was dried over anhydrous sodium sulfate and concentrated under vacuum. The residue was purified by silica gel chromatography using 0 to 30% ethyl acetate in hexane to furnish tert-butyl N-tert-butoxycarbonyl-N-[4-chloro-5-methyl-6-[2-methyl-6-(tetrahydropyran-4-ylidenemethyl)phenyl]pyrimidin-2-yl]carbamate (823 mg, 74%) as an off-white foam. ESI-MS m/z calc. 529.2344, found 530.2 (M+1)⁺; Retention time: 4.17 minutes; LC Method T.

### Step 6: tert-Butyl N-tert-butoxycarbonyl-N-[4-chloro-5-methyl-6-[2-methyl-6-(tetrahydropyran-4-ylmethyl)phenyl]pyrimidin-2-yl]carbamate

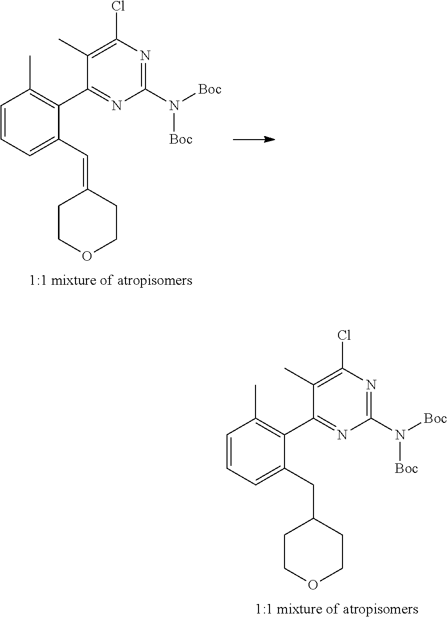 [SMILES: Cc1cccc(C=C2CCOCC2)c1-c1nc(N(C(=O)OC(C)(C)C)C(=O)OC(C)(C)C)nc(Cl)c1C ; Cc1cccc(CC2CCOCC2)c1-c1nc(N(C(=O)OC(C)(C)C)C(=O)OC(C)(C)C)nc(Cl)c1C]

To a solution of tert-butyl N-tert-butoxycarbonyl-N-[4-chloro-5-methyl-6-[2-methyl-6-(tetrahydropyran-4-ylidenemethyl)phenyl]pyrimidin-2-yl]carbamate (1.145 g, 2.1601 mmol) in ethyl acetate (50 mL) was added 5% rhodium on alumina (445 mg, 5% w/w, 0.2162 mmol). The reaction was hydrogenated in a Parr shaker for 3 h at 50 psi hydrogen atmosphere. The catalyst was filtered off through a pad of Celite. The filtrate was concentrated under vacuum to furnish tert-butyl N-tert-butoxycarbonyl-N-[4-chloro-5-methyl-6-[2-methyl-6-(tetrahydropyran-4-ylmethyl)phenyl]pyrimidin-2-yl]carbamate (1.104 g, 93%) as a white foam. ESI-MS m/z calc. 531.25, found 532.2 (M+1)⁺; Retention time: 4.2 minutes; LC Method T.

### Step 7: 4-Chloro-5-methyl-6-[2-methyl-6-(tetrahydropyran-4-ylmethyl)phenyl]pyrimidin-2-amine

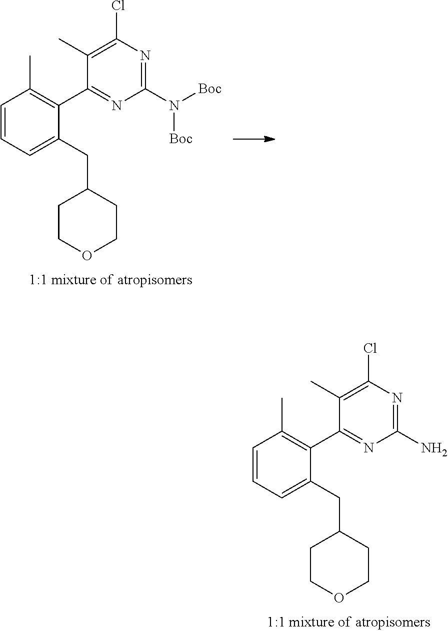 [SMILES: Cc1cccc(CC2CCOCC2)c1-c1nc(N(C(=O)OC(C)(C)C)C(=O)OC(C)(C)C)nc(Cl)c1C ; Cc1cccc(CC2CCOCC2)c1-c1nc(N)nc(Cl)c1C]

To a solution of tert-butyl N-tert-butoxycarbonyl-N-[4-chloro-5-methyl-6-[2-methyl-6-(tetrahydropyran-4-ylmethyl)phenyl]pyrimidin-2-yl]carbamate (1.104 g, 2.0127 mmol) in DCM (10 mL) was added 2.0 M HCl in ether (10 mL of 2 M, 20.0 mmol) at rt. The reaction was stirred at rt overnight. The volatiles were removed under vacuum. The residue was dissolved in DCM (50 mL). The organic solution was washed with saturated sodium bicarbonate (50 mL) and dried over anhydrous sodium sulfate. After concentration under vacuum, the crude material was purified by silica gel chromatography using 0 to 30% acetone in hexane to furnish 4-chloro-5-methyl-6-[2-methyl-6-(tetrahydropyran-4-ylmethyl)phenyl]pyrimidin-2-amine (575 mg, 84%) as a white solid. ¹H NMR (500 MHz, DMSO-d₆) δ 7.24 (t, J=7.6, 7.6 Hz, 1H), 7.12 (dd, J=12.6, 7.5 Hz, 2H), 6.92 (s, 2H), 3.80-3.64 (m, 2H), 3.14 (td, J=11.6, 11.6, 2.3 Hz, 2H), 2.30 (dd, J=13.6, 7.4 Hz, 1H), 2.11 (dd, J=13.6, 6.9 Hz, 1H), 1.96 (s, 3H), 1.77 (s, 3H), 1.71-1.54 (m, 1H), 1.39-1.31 (m, 1H), 1.31-1.22 (m, 1H), 1.12-0.97 (m, 2H). ESI-MS m/z calc. 331.14514, found 332.5 (M+1)⁺; Retention time: 2.25 minutes; LC Method W.

### Step 8: Methyl 3-[[4-chloro-5-methyl-6-[2-methyl-6-(tetrahydropyran-4-ylmethyl)phenyl]pyrimidin-2-yl]sulfamoyl]benzoate

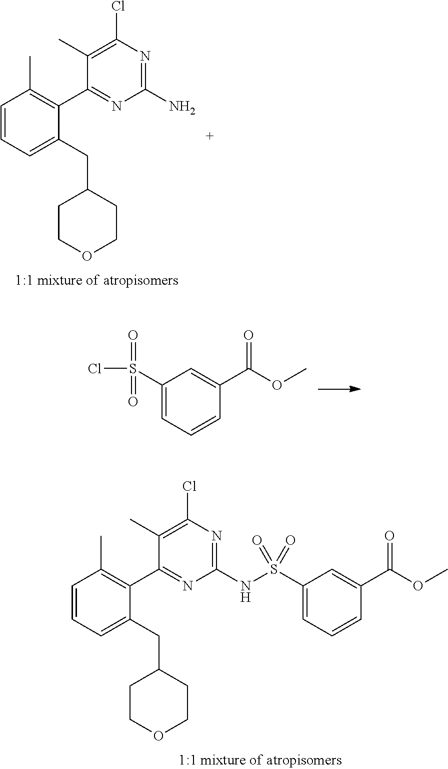 [SMILES: Cc1cccc(CC2CCOCC2)c1-c1nc(N)nc(Cl)c1C ; COC(=O)c1cccc(S(=O)(=O)Cl)c1 ; COC(=O)c1cccc(S(=O)(=O)Nc2nc(Cl)c(C)c(-c3c(C)cccc3CC3CCOCC3)n2)c1]

To a solution of 4-chloro-5-methyl-6-[2-methyl-6-(tetrahydropyran-4-ylmethyl)phenyl]pyrimidin-2-amine (519 mg, 1.5640 mmol) and methyl 3-chlorosulfonylbenzoate (1.1 g, 4.6877 mmol) in anhydrous THF (10 mL) was added 1.3 M LiHMDS in THF (4.8 mL of 1.3 M, 6.24 mmol) dropwise at −78° C. The reaction was slowly warmed to rt and stirred for 2 h. The reaction was quenched with 10% aq. citric acid (30 mL) and extracted with ethyl acetate (3×30 mL). The combined organic layers were washed with brine (30 mL), dried over anhydrous sodium sulfate and concentrated under vacuum. The residue was purified by silica gel chromatography using 0 to 50% acetone in hexane to furnish methyl 3-[[4-chloro-5-methyl-6-[2-methyl-6-(tetrahydropyran-4-ylmethyl)phenyl]pyrimidin-2-yl]sulfamoyl]benzoate (786 mg, 93%) as a white foam. ESI-MS m/z calc. 529.1438, found 530.2 (M+1)⁺; Retention time: 3.54 minutes; LC Method T.

### Step 9: 3-[[4-Chloro-5-methyl-6-[2-methyl-6-(tetrahydropyran-4-ylmethyl)phenyl]pyrimidin-2-yl]sulfamoyl]benzoic acid

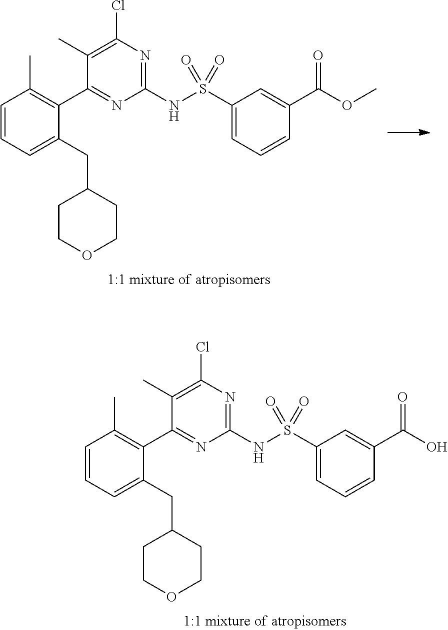 [SMILES: COC(=O)c1cccc(S(=O)(=O)Nc2nc(Cl)c(C)c(-c3c(C)cccc3CC3CCOCC3)n2)c1 ; Cc1cccc(CC2CCOCC2)c1-c1nc(NS(=O)(=O)c2cccc(C(=O)O)c2)nc(Cl)c1C]

To a solution of methyl 3-[[4-chloro-5-methyl-6-[2-methyl-6-(tetrahydropyran-4-ylmethyl)phenyl]pyrimidin-2-yl]sulfamoyl]benzoate (786 mg, 1.4533 mmol) in THF (7 mL) was added 1 N NaOH solution (7 mL of 1 M, 7.0 mmol). The reaction was stirred at rt for 3 h. The reaction was diluted with 1 N HCl (30 mL) and ethyl acetate (30 mL). The layers were separated and the aqueous layer was extracted with ethyl acetate (2×30 mL). The combined organic layers were washed with brine (30 mL), dried over anhydrous sodium sulfate and concentrated under vacuum to furnish 3-[[4-chloro-5-methyl-6-[2-methyl-6-(tetrahydropyran-4-ylmethyl)phenyl]pyrimidin-2-yl]sulfamoyl]benzoic acid (765 mg, 100%) as a white solid. ESI-MS m/z calc. 515.1282, found 516.1 (M+1)⁺; Retention time: 3.11 minutes; LC Method T.

### Step 10: 3-[[4-[(2R)-2-amino-4,4-dimethyl-pentoxy]-5-methyl-6-[2-methyl-6-(tetrahydropyran-4-ylmethyl)phenyl]pyrimidin-2-yl]sulfamoyl]benzoic acid

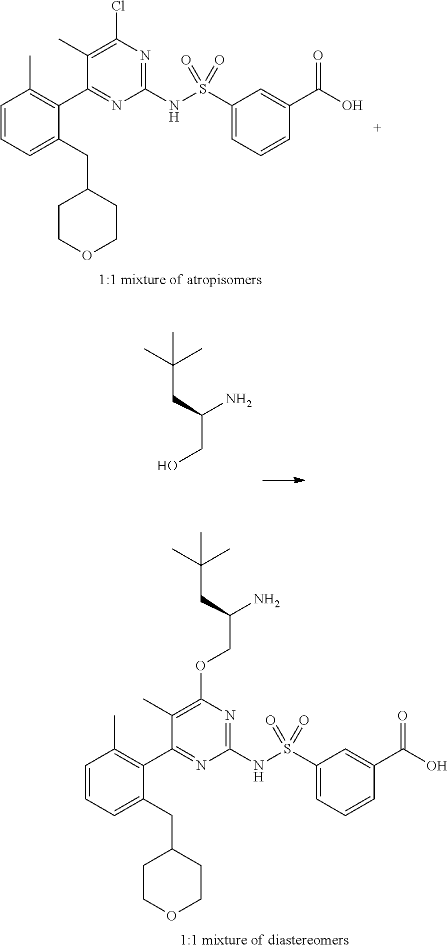

To a solution of 3-[[4-chloro-5-methyl-6-[2-methyl-6-(tetrahydropyran-4-ylmethyl)phenyl]pyrimidin-2-yl]sulfamoyl]benzoic acid (765 mg, 1.4529 mmol) and (2R)-2-amino-4,4-dimethyl-pentan-1-ol (hydrochloride salt) (372 mg, 2.2186 mmol) in anhydrous THE (15 mL) was added sodium tert-butoxide (697 mg, 7.2526 mmol) at rt. The reaction was stirred at rt for 2 h. The reaction was quenched with 1 N HCl (15 mL) in an ice bath. The reaction was then extracted with ethyl acetate (3×30 mL). The combined organic layers were washed with brine (30 mL), dried over anhydrous sodium sulfate and concentrated under vacuum. The crude material was purified by silica gel chromatography using 0 to 10% methanol in chloroform (buffered with 0.3% acetic acid) to furnish 3-[[4-[(2R)-2-amino-4,4-dimethyl-pentoxy]-5-methyl-6-[2-methyl-6-(tetrahydropyran-4-ylmethyl)phenyl]pyrimidin-2-yl]sulfamoyl]benzoic acid (hydrochloride salt) (623 mg, 60%) as a white solid. ¹H NMR (500 MHz, DMSO-d₆) δ 8.65 (d, J=9.0 Hz, 1H), 8.05 (d, J=7.4 Hz, 1H), 7.82 (d, J=7.9 Hz, 1H), 7.50 (d, J=7.7 Hz, 1H), 7.25 (t, J=7.6, 7.6 Hz, 1H), 7.11 (apparent dd, J=16.6, 7.6 Hz, 2H), 4.60-4.50 (m, 1H), 4.50-4.41 (m, 1H), 4.37 (dd, J=11.4, 6.3 Hz, 1H), 3.76-3.66 (m, 4H), 3.15-3.06 (m, 4H), 2.50 (s, 3H), 2.22-2.10 (m, 1H), 2.06-1.98 (m, 1H), 1.88 (s, 3H), 1.73-1.46 (m, 4H), 1.26 (d, J=13.0 Hz, 1H), 1.10 (d, J=13.1 Hz, 1H), 1.04-0.95 (m, 1H), 0.91 (s, 9H). ESI-MS m/z calc. 610.28253, found 611.5 (M+1)⁺; Retention time: 1.91 minutes; LC Method W.

### Step 11: 3-[[4-[(2R)-4,4-Dimethyl-2-[(5-morpholinopyrimidin-2-yl)methylamino]pentoxy]-5-methyl-6-[2-methyl-6-(tetrahydropyran-4-ylmethyl)phenyl]pyrimidin-2-yl]sulfamoyl]benzoic acid

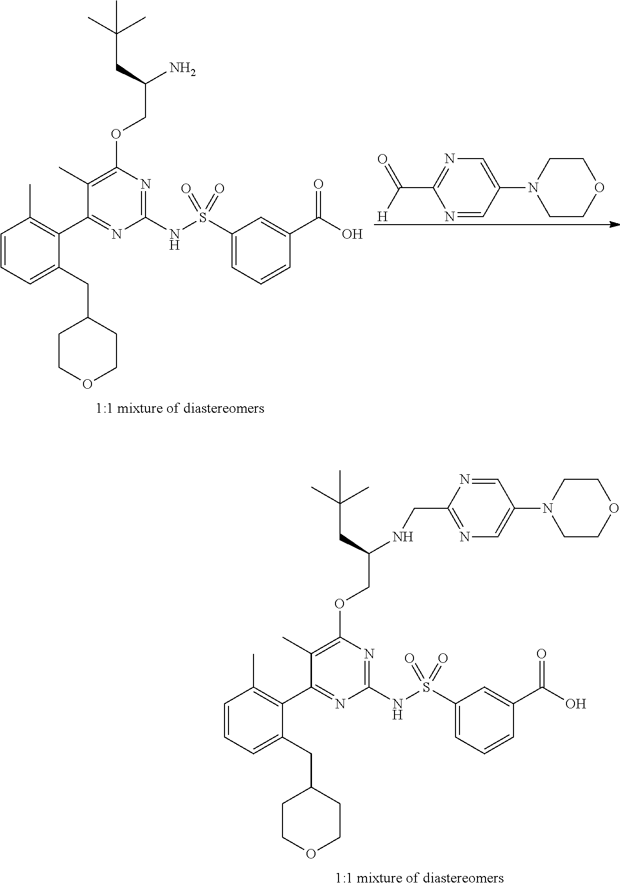

A 4-mL vial was charged with 3-[[4-[(2R)-2-amino-4,4-dimethyl-pentoxy]-5-methyl-6-[2-methyl-6-(tetrahydropyran-4-ylmethyl)phenyl]pyrimidin-2-yl]sulfamoyl]benzoic acid (hydrochloride salt) (103 mg, 0.1591 mmol), 5-morpholinopyrimidine-2-carbaldehyde (34 mg, 0.1672 mmol) in DCE (1 mL). The reaction was stirred at rt for 15 min. Sodium triacetoxyborohydride (50 mg, 0.2359 mmol) was added and the reaction mixture was stirred at rt for 1 h. The reaction was quenched with 1 N HCl (20 mL) and extracted with ethyl acetate (3×20 mL). The combined organic layers were washed with brine (20 mL), dried over anhydrous sodium sulfate and concentrated under vacuum to furnish 3-[[4-[(2R)-4,4-dimethyl-2-[(5-morpholinopyrimidin-2-yl)methylamino]pentoxy]-5-methyl-6-[2-methyl-6-(tetrahydropyran-4-ylmethyl)phenyl]pyrimidin-2-yl]sulfamoyl]benzoic acid (hydrochloride salt) (114 mg, 78%) as an off-white solid. ESI-MS m/z calc. 787.37274, found 788.6 (M+1)⁺; Retention time: 2.63 minutes; LC Method T.

### Step 12: (11R)-11-(2,2-dimethylpropyl)-7-methyl-6-[2-methyl-6-(tetrahydropyran-4-ylmethyl)phenyl]-12-[(5-morpholinopyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one

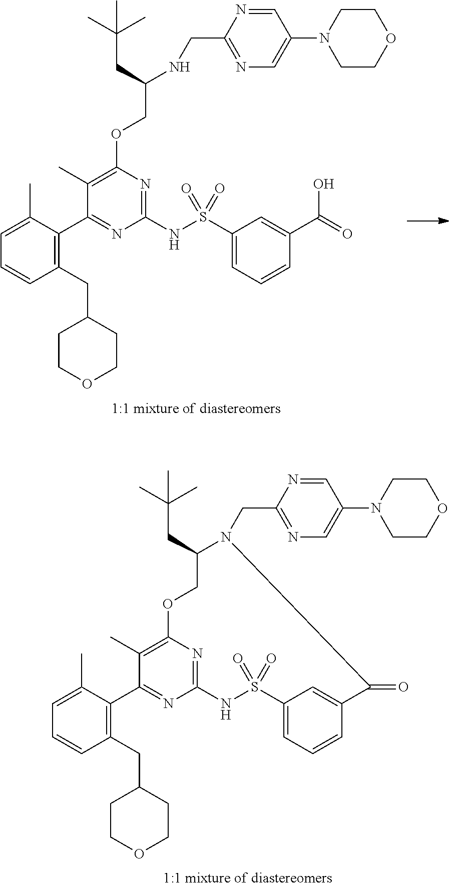 [SMILES: Cc1cccc(CC2CCOCC2)c1-c1nc(NS(=O)(=O)c2cccc(C(=O)O)c2)nc(OC[C@@H](CC(C)(C)C)NCc2ncc(N3CCOCC3)cn2)c1C ; Cc1cccc(CC2CCOCC2)c1-c1nc2nc(c1C)OC[C@@H](CC(C)(C)C)N(Cc1ncc(N3CCOCC3)cn1)C(=O)c1cccc(c1)S(=O)(=O)N2]

To a solution of 3-[[4-[(2R)-4,4-dimethyl-2-[(5-morpholinopyrimidin-2-yl)methylamino]pentoxy]-5-methyl-6-[2-methyl-6-(tetrahydropyran-4-ylmethyl)phenyl]pyrimidin-2-yl]sulfamoyl]benzoic acid (hydrochloride salt) (114 mg, 0.1244 mmol) and CDMT (34 mg, 0.1937 mmol) in anhydrous DMF (3 mL) was added NMM (56.120 mg, 0.061 mL, 0.5548 mmol) at 0° C. The reaction was stirred at rt for 3 h. Another portion of CDMT (51 mg, 0.2905 mmol) and NMM (56.120 mg, 0.061 mL, 0.5548 mmol) was added. The reaction was stirred at rt overnight. The reaction was quenched with 10% aqueous citric acid (20 mL) and the reaction was extracted with ethyl acetate (3×20 mL). The combined organic layers were washed with brine (3×20 mL), dried over anhydrous sodium sulfate and concentrated under vacuum. The residue was purified by silica gel chromatography using 0 to 5% methanol in DCM, then purified again with reverse-phase HPLC using 0 to 100% acetonitrile in water (buffered with 0.1 M HCl) to furnish (11R)-11-(2,2-dimethylpropyl)-7-methyl-6-[2-methyl-6-(tetrahydropyran-4-ylmethyl)phenyl]-12-[(5-morpholinopyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (24.6 mg, 25%) as a white powder. ¹H NMR (500 MHz, DMSO-d₆) δ 13.06 (s, 1H), 8.81-8.61 (m, 1H), 8.59-8.43 (m, 2H), 7.92 (d, J=18.7 Hz, 1H), 7.67 (s, 2H), 7.30 (s, 1H), 7.16 (dd, J=29.2, 8.0 Hz, 2H), 5.43 (d, J=10.5 Hz, 1H), 4.86 (dd, J=16.4, 11.0 Hz, 1H), 4.61 (dd, J=16.4, 5.2 Hz, 1H), 4.18 (q, J=11.7, 11.5, 11.5 Hz, 1H), 4.06-3.86 (m, 1H), 3.78-3.65 (m, 6H), 3.26-3.20 (m, 4H), 3.18-3.02 (m, 2H), 2.37-2.18 (m, 1H), 2.04 (s, 2H), 1.91-1.71 (m, 3H), 1.60 (s, 4H), 1.41-1.24 (m, 3H), 1.17-1.02 (m, 1H), 0.96-0.75 (m, 1H), 0.62-0.52 (m, 9H). ESI-MS m/z calc. 769.3622, found 770.5 (M+1)⁺; Retention time: 2.51 minutes; LC Method W.

### Step 13: (5M,11R)-11-(2,2-dimethylpropyl)-7-methyl-6-{2-methyl-6-[(oxan-4-yl)methyl]phenyl}-12-{[5-(morpholin-4-yl)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (Compound 217) and (5P,11R)-11-(2,2-dimethylpropyl)-7-methyl-6-{2-methyl-6-[(oxan-4-yl)methyl]phenyl}-12-{[5-(morpholin-4-yl)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (Compound 218)

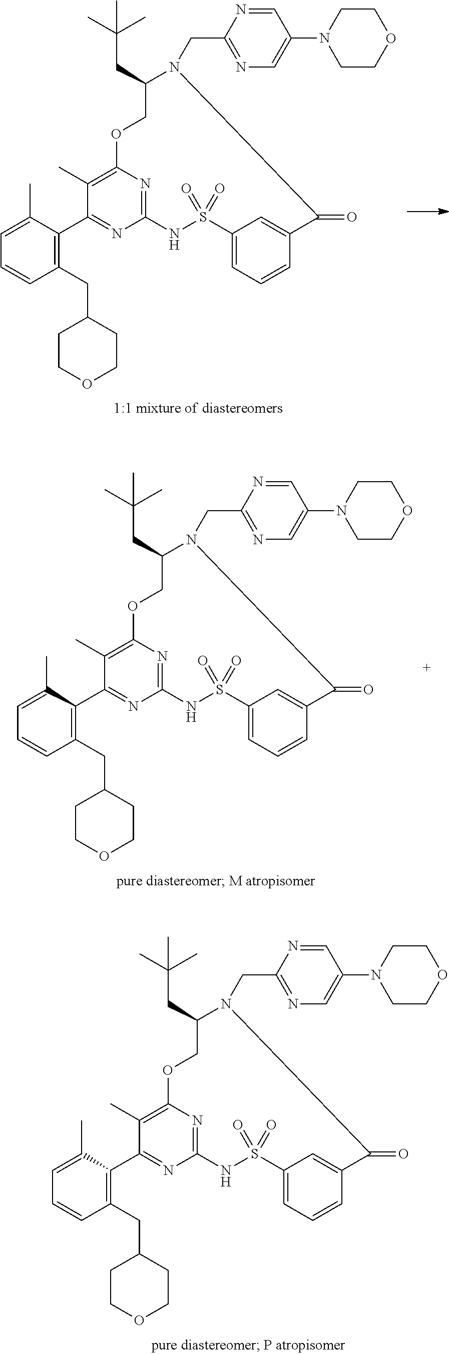

(11R)-11-(2,2-Dimethylpropyl)-7-methyl-6-{2-methyl-6-[(oxan-4-yl)methyl]phenyl}-12-{[5-(morpholin-4-yl)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (24.6 mg, 0.03195 mmol) (1:1 mixture of diastereomers) was diluted in DMSO. Purification by reverse-phase HPLC (1-99% acetonitrile/5 mM HCl) provided two isomers as yellow solids. More polar, first-to-elute isomer: (5M,11R)-11-(2,2-dimethylpropyl)-7-methyl-6-{2-methyl-6-[(oxan-4-yl)methyl]phenyl}-12-{[5-(morpholin-4-yl)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (8.9 mg, 68%). ¹H NMR (400 MHz, DMSO-d₆) δ 8.70 (s, 1H), 8.50 (s, 2H), 7.90 (d, J=7.2 Hz, 1H), 7.72-7.60 (m, 2H), 7.30 (t, J=7.6 Hz, 1H), 7.19 (d, J=7.5 Hz, 1H), 7.11 (d, J=7.7 Hz, 1H), 5.43 (dd, J=10.7, 4.3 Hz, 1H), 4.85 (d, J=16.3 Hz, 1H), 4.62 (d, J=16.5 Hz, 1H), 4.17 (t, J=11.1 Hz, 1H), 3.97-3.91 (m, 1H), 3.77-3.73 (overlapped with water, m, 6H), 3.27-3.19 (m, 4H), 3.17-3.00 (m, 2H), 2.06-1.97 (m, 4H), 1.93-1.79 (m, 2H), 1.60 (s, 3H), 1.55-1.43 (m, 1H), 1.37 (d, J=15.0 Hz, 1H), 1.27 (d, J=13.0 Hz, 1H), 1.05-0.72 (m, 3H), 0.59 (s, 9H) (NH signal around 13 ppm too flat for accurate integration). ESI-MS m/z calc. 769.3622, found 770.65 (M+1)⁺; Retention time: 1.73 minutes; LC Method A. Less polar, second-to-elute isomer: (5P,11R)-11-(2,2-dimethylpropyl)-7-methyl-6-{2-methyl-6-[(oxan-4-yl)methyl]phenyl}-12-{[5-(morpholin-4-yl)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (9.6 mg, 72%). ¹H NMR (400 MHz, DMSO-d₆) δ 8.71 (s, 1H), 8.50 (s, 2H), 7.94 (d, J=7.4 Hz, 1H), 7.73-7.61 (m, 2H), 7.31 (t, J=7.6 Hz, 1H), 7.18 (d, J=7.7 Hz, 1H), 7.13 (d, J=7.6 Hz, 1H), 5.43 (dd, J=10.8, 4.3 Hz, 1H), 4.87 (d, J=16.4 Hz, 1H), 4.61 (d, J=16.5 Hz, 1H), 4.20 (overlapped with water, t, J=11.2 Hz, 1H), 3.80-3.73 (overlapped with water, m, 6H), 3.26-3.14 (m, 6H), 2.36-2.19 (m, 2H), 1.82 (dd, J=15.2, 8.8 Hz, 1H), 1.77 (s, 3H), 1.72-1.63 (m, 1H), 1.60 (s, 3H), 1.42-1.32 (m, 3H), 1.16-1.00 (m, 2H), 0.55 (s, 9H). (NH signal around 13 ppm too flat for accurate integration. One H signal overlapped with water around 4 ppm). ESI-MS m/z calc. 769.3622, found 770.82 (M+1)⁺; Retention time: 1.78 minutes; LC Method A.

### Example 77: Preparation of Compound 219

### Step 1: (5P)-4-chloro-5-methyl-6-[2-methyl-6-(2-methylpropyl)phenyl]pyrimidin-2-amine and (5M)-4-chloro-5-methyl-6-[2-methyl-6-(2-methylpropyl)phenyl]pyrimidin-2-amine

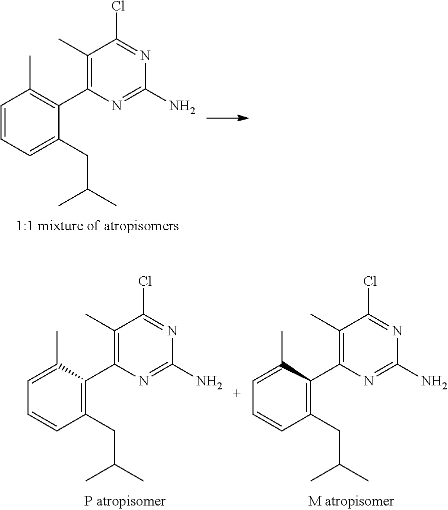

Racemic 4-chloro-6-(2-isobutyl-6-methyl-phenyl)-5-methyl-pyrimidin-2-amine (13.91 g, 48.00 mmol) was subjected to chiral SFC separation using the following conditions: IC column (3×25 cm), eluant: 10% MeOH/90% CO₂, 100 bar, flow rate: 80 mL/min, 220 nm, injection volume 0.5 mL, 5 mg/mL in methanol:DCM. Two atropisomers were isolated. SFC peak 1: (5P)-4-chloro-5-methyl-6-[2-methyl-6-(2-methylpropyl)phenyl]pyrimidin-2-amine (6.12 g, 88%). ESI-MS m/z calc. 289.13458, found 290.31 (M+1)⁺; Retention time: 1.84 minutes; LC Method A. SFC peak 2: (5M)-4-chloro-5-methyl-6-[2-methyl-6-(2-methylpropyl)phenyl]pyrimidin-2-amine (6.14 g, 88%). ESI-MS m/z calc. 289.13458, found 290.31 (M+1)⁺; Retention time: 1.85 minutes; LC Method A.

### Step 2: (5P)-Methyl 3-[[4-chloro-6-(2-isobutyl-6-methyl-phenyl)-5-methyl-pyrimidin-2-yl]sulfamoyl]benzoate

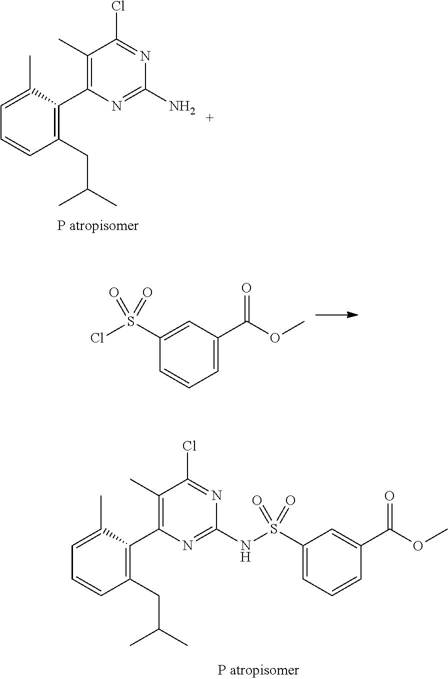

To a solution of 4-chloro-6-(2-isobutyl-6-methyl-phenyl)-5-methyl-pyrimidin-2-amine (6 g, 20.704 mmol) and methyl 3-chlorosulfonylbenzoate (14.6 g, 62.218 mmol) in THF (120 mL) was added a solution of LiHMDS in THF (83 mL of 1.0 M, 83.0 mmol) dropwise over 20 min at −78° C. The reaction mixture was stirred for 2 h and then quenched with 1 N HCl solution (100 mL). The layers were separated and the aqueous layer was extracted with ethyl acetate (2×250 mL). The combined organic layers were washed with brine, dried over sodium sulfate, filtered and concentrated in vacuo. The resulting yellow oil was purified silica gel chromatography (120 g silica, eluted with 10% ethyl acetate in hexanes) to give methyl 3-[[4-chloro-6-(2-isobutyl-6-methyl-phenyl)-5-methyl-pyrimidin-2-yl]sulfamoyl]benzoate (9.6 g, 86%) as a yellow sticky solid. ESI-MS m/z calc. 487.13324, found 488.8 (M+1)⁺; Retention time: 7.18 minutes; LC Method S.

### Step 3: (5P)-3-[[4-Chloro-6-(2-isobutyl-6-methyl-phenyl)-5-methyl-pyrimidin-2-yl]sulfamoyl]benzoic acid

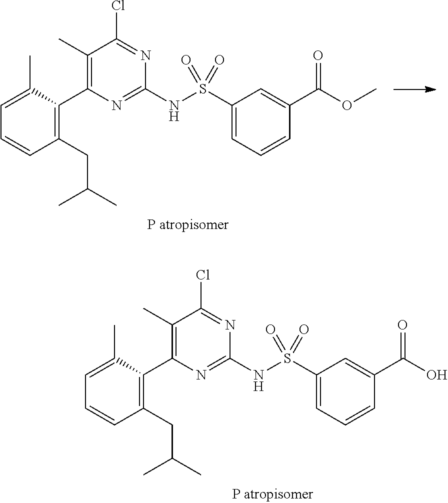

To a stirred solution of (5P)-methyl 3-[[4-chloro-6-(2-isobutyl-6-methyl-phenyl)-5-methyl-pyrimidin-2-yl]sulfamoyl]benzoate (9.6 g, 18.689 mmol) in THF (100 mL) was added aqueous NaOH solution (100 mL of 1 M, 100.0 mmol) at rt and stirred for 2 h. The reaction mixture was extracted with ethyl acetate (2×250 mL). The aqueous layer was acidified with 1 N HCl solution to pH=1 and then extracted with ethyl acetate (2×250 mL). The combined organic layer was washed with brine, dried over sodium sulfate, filtered and concentrated in vacuo to give 3-[[4-chloro-6-(2-isobutyl-6-methyl-phenyl)-5-methyl-pyrimidin-2-yl]sulfamoyl]benzoic acid (8.74 g, 97%) as a white solid. ESI-MS m/z calc. 473.1176, found 474.4 (M+1)⁺; Retention time: 6.69 minutes; LC Method S.

### Step 4: (5P)-3-[[4-[(2R)-2-Amino-4-methyl-pentoxy]-6-(2-isobutyl-6-methyl-phenyl)-5-methyl-pyrimidin-2-yl]sulfamoyl]benzoic acid

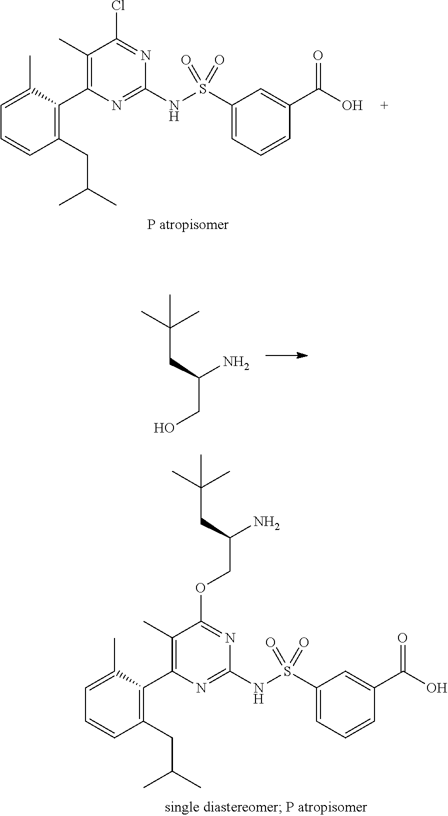

A solution of (5P)-3-[[4-chloro-6-(2-isobutyl-6-methyl-phenyl)-5-methyl-pyrimidin-2-yl]sulfamoyl]benzoic acid (200 mg, 0.4009 mmol), (2R)-2-amino-4-methyl-pentan-1-ol (120 mg, 1.0240 mmol) and sodium t-butoxide (210 mg, 2.1851 mmol) in THF (4.0 mL) was stirred at rt for 2 h. The reaction mixture was quenched with 1 N HCl solution and extracted with ethyl acetate (3×25 mL). The combined organic layer was washed with brine, dried over sodium sulfate, filtered and concentrated in vacuo to give (5P)-3-[[4-[(2R)-2-amino-4-methyl-pentoxy]-6-(2-isobutyl-6-methyl-phenyl)-5-methyl-pyrimidin-2-yl]sulfamoyl]benzoic acid (hydrochloride salt) (176 mg, 65%). ¹H NMR (500 MHz, DMSO-d₆) δ 13.11 (bs, 1H), 8.44-8.38 (m, 1H), 8.18 (s, 3H), 8.10 (dd, J=17.5, 7.8 Hz, 2H), 7.66 (t, J=7.8 Hz, 1H), 7.28 (s, 1H), 7.14 (d, J=11.4 Hz, 2H), 4.30 (d, J=11.8 Hz, 1H), 4.23-4.08 (m, 1H), 3.62-3.51 (m, 1H), 2.16-2.06 (m, 1H), 1.96-1.81 (m, 4H), 1.65 (s, 3H), 1.55-1.50 (m, 1H), 1.49-1.44 (m, 2H), 0.93-0.88 (m, 6H), 0.77-0.56 (m, 6H). ESI-MS m/z calc. 554.2563, found 555.4 (M+1)⁺; Retention time: 2.01 minutes; LC Method W.

### Step 5: Methyl 5-isobutylpyrimidine-2-carboxylate

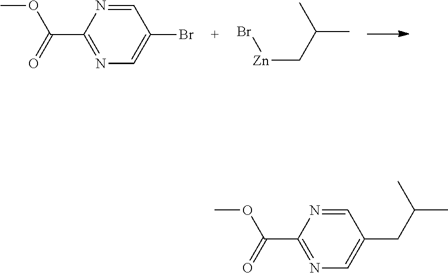

To a mixture of methyl 5-bromopyrimidine-2-carboxylate (12 g, 55.294 mmol) in anhydrous THF (200 mL) was added Pd(dppf)Cl₂ (4.52 g, 5.5349 mmol). A solution of bromo(isobutyl)zinc in THF (145 mL of 0.5 M, 72.50 mmol) was then added dropwise at RT. After the addition, the reaction mixture was stirred at RT for 4 h. The reaction was then concentrated under reduced pressure. The crude was then purified via silica gel column chromatography (eluting with 20-100% EtOAc in hexanes) to yield methyl 5-isobutylpyrimidine-2-carboxylate (3.5 g, 32%) as a brown oil. ESI-MS m/z calc. 194.10553, found 195.3 (M+1)⁺; Retention time: 2.13 minutes; LC Method T.

### Step 6: (5-Isobutylpyrimidin-2-yl)methanol

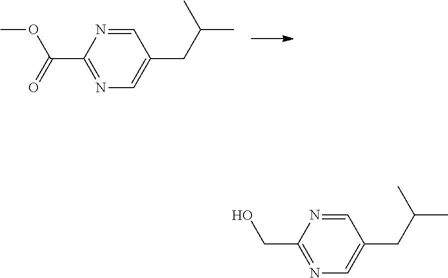

To a solution of methyl 5-isobutylpyrimidine-2-carboxylate (3.5 g, 18.020 mmol) in MeOH (70 mL) was added sodium borohydride (1.15 g, 30.397 mmol). The reaction was stirred at RT for 1.5 h. The reaction was quenched with water (100 mL) and partitioned with EtOAc (70 mL). The aqueous layer was extracted with EtOAc (2×50 mL). The organic layers were combined, washed with brine, dried with anhydrous sodium sulfate, filtered, and concentrated under reduced pressure. The crude product was purified via silica gel column chromatography (eluting with 0-70% EtOAc in hexanes) to yield (5-isobutylpyrimidin-2-yl)methanol (2.5 g, 83%) as a yellow oil. ESI-MS m/z calc. 166.11061, found 166.9 (M+1)⁺; Retention time: 1.76 minutes; LC Method T.

### Step 7: 5-Isobutylpyrimidine-2-carbaldehyde

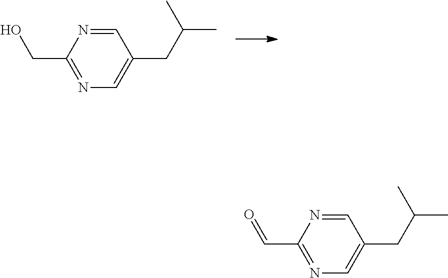

To a solution of (5-isobutylpyrimidin-2-yl)methanol (2.47 g, 14.117 mmol) in DCM (50 mL) was added DMP (7.19 g, 16.952 mmol) and stirred at RT for 1.5 h. The reaction was then filtered over a pad of Celite and the filtrate was concentrated under reduced pressure. The crude was then purified via silica gel column chromatography (eluting with 0-10% MeOH in DCM) to yield crude product. The crude product was then dissolved in EtOAc (25 mL) and washed with 1 N NaOH solution (25 mL). The aqueous layer was then extracted with EtOAc (3×30 mL). The organic layers were combined, dried with anhydrous sodium sulfate, filtered, and concentrated under reduced pressure to yield 5-isobutylpyrimidine-2-carbaldehyde (1.9 g, 80%) as a light yellow oil. ¹H NMR (500 MHz, DMSO-d₆) δ 9.95 (s, 1H), 8.92 (s, 2H), 2.59 (d, J=7.2 Hz, 2H), 1.93 (dt, J=13.6, 6.8 Hz, 1H), 0.88 (s, 3H), 0.87 (s, 3H). ESI-MS m/z calc. 164.09496, found 165.1 (M+1)⁺; Retention time: 1.24 minutes; LC Method W.

### Step 8: (5P)-3-[[4-(2-Isobutyl-6-methyl-phenyl)-6-[(2R)-2-[(5-isobutylpyrimidin-2-yl)methylamino]-4-methyl-pentoxy]-5-methyl-pyrimidin-2-yl]sulfamoyl]benzoic acid

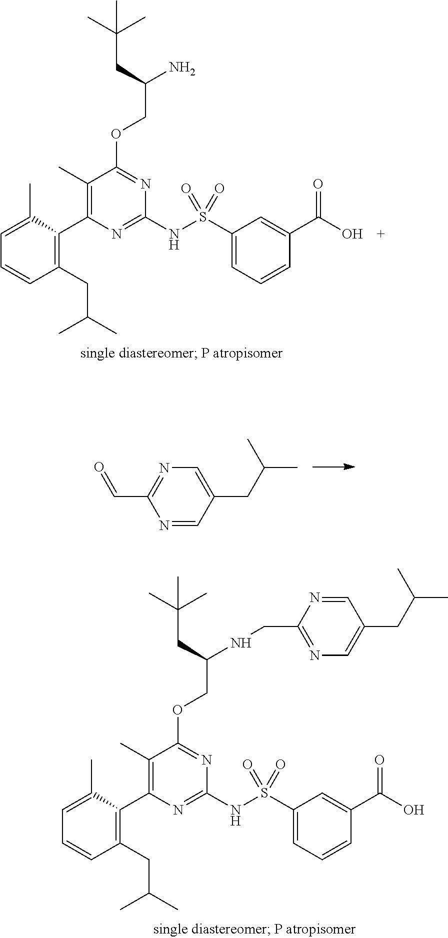

To a 0° C. solution of (5P)-3-[[4-[(2R)-2-amino-4-methyl-pentoxy]-6-(2-isobutyl-6-methyl-phenyl)-5-methyl-pyrimidin-2-yl]sulfamoyl]benzoic acid (hydrochloride salt) (30 mg, 0.0445 mmol) and 5-isobutylpyrimidine-2-carbaldehyde (10 mg, 0.0510 mmol) in dry dichloromethane (5 mL) was added sodium triacetoxyborohydride (45 mg, 0.2123 mmol). After 5 min at 0° C., the reaction was stirred at room temperature for 1 h. The reaction was quenched at 0° C. by adding aqueous 1 N HCl solution (5 mL) and the mixture was stirred for 30 min at 0° C. The phases were separated and the aqueous layer was extracted with EtOAc (3×20 mL). The combined organic layers were dried over sodium sulfate, filtered and concentrated in vacuo to afford crude (5P)-3-[[4-(2-isobutyl-6-methyl-phenyl)-6-[(2R)-2-[(5-isobutylpyrimidin-2-yl)methylamino]-4-methyl-pentoxy]-5-methyl-pyrimidin-2-yl]sulfamoyl]benzoic acid (hydrochloride salt) (40 mg, 65%) as a yellow solid. ESI-MS m/z calc. 702.3563, found 703.2 (M+1)⁺; Retention time: 1.58 minutes; LC Method X.

### Step 9: (5P,11R)-11-isobutyl-6-(2-isobutyl-6-methyl-phenyl)-12-[(5-isobutylpyrimidin-2-yl)methyl]-7-methyl-2,2-dioxo-9-oxa-2⁶-thia-3,5,12,19-tetrazatricyclo[12.3.1.14,8]nonadeca-1(18),4(19),5,7,14,16-hexaen-13-one (Compound 219)

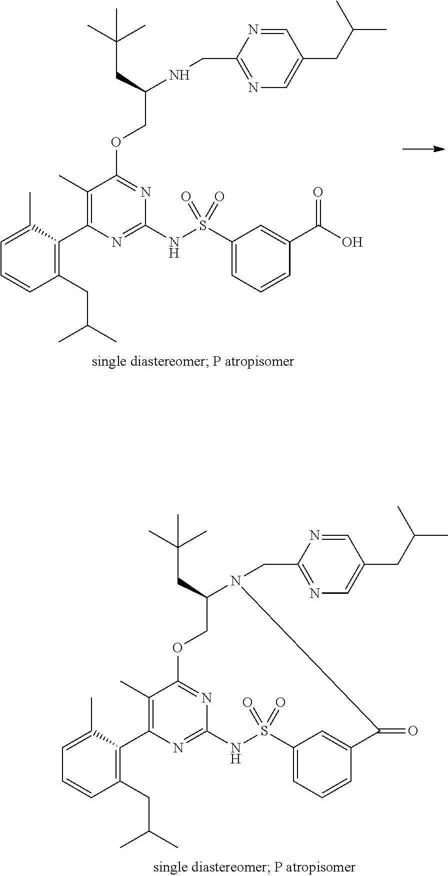

To a 0° C. solution of (5P)-3-[[4-(2-isobutyl-6-methyl-phenyl)-6-[(2R)-2-[(5-isobutylpyrimidin-2-yl)methylamino]-4-methyl-pentoxy]-5-methyl-pyrimidin-2-yl]sulfamoyl]benzoic acid (hydrochloride salt) (40 mg, 0.0289 mmol) in DMF (5 mL) was added N-methylmorpholine (36.8 mg, 40 μL, 0.3638 mmol) followed by 2-chloro-4,6-dimethoxy-1,3,5-triazine (12 mg, 0.0683 mmol). The mixture was stirred at 0° C. for 5 min and then stirred at room temperature for 72 h. The reaction mixture was concentrated in vacuo at 50° C. and the crude product was purified by reverse-phase HPLC (C₁₈ column), eluting with a gradient of MeCN in acidic water (0.1% v/v of formic acid in water) to afford (5P, 11R)-11-isobutyl-6-(2-isobutyl-6-methyl-phenyl)-12-[(5-isobutylpyrimidin-2-yl)methyl]-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (3 mg, 15%) as a white solid. ¹H NMR (400 MHz, DMSO-d₆) δ 12.99 (br. s., 1H), 8.67 (br. s., 1H), 8.64 (s, 2H), 7.90 (br. s., 1H), 7.75-7.56 (m, 2H), 7.31 (br. s., 1H), 7.22-7.07 (m, 2H), 5.44 (br. s., 1H), 4.92 (d, J=16.6 Hz, 1H), 4.69 (d, J=16.9 Hz, 1H), 4.35-4.18 (m, 1H), 4.06-3.91 (m, 1H), 2.25-2.18 (m, 2H), 1.94-1.86 (m, 1H), 1.84-1.67 (m, 6H), 1.57 (br. s., 3H), 1.39 (br. s., 1H), 1.24-1.16 (m, 1H), 0.88 (d, J=6.6 Hz, 6H), 0.81-0.72 (m, 1OH), 0.25 (br. s., 3H). ESI-MS m/z calc. 684.34576, found 685.3 (M+1)⁺; Retention time: 5.06 minutes; LC Method Y.

### Example 78: Preparation of Compound 220 and Compound 221

### Step 1: 2-Bromo-1-(isopropoxymethyl)-3-methyl-benzene

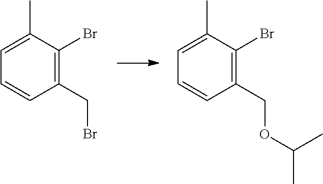

A slurry of NaH (13.1 g, 327.53 mmol) in THF (600 mL) at 0° C. was added isopropanol (23.550 g, 30 mL, 391.88 mmol) slowly, and then stirred for 30 min. A solution of 2-bromo-1-(bromomethyl)-3-methyl-benzene (45.5 g, 172.38 mmol) in THF (200 mL) was added to the reaction mixture. It was then warmed to room temperature, and then stirred overnight. The reaction was quenched with saturated NH₄Cl (400 mL), and extracted with Et₂O (3×250 mL). The combined organic extracts were dried over anhydrous sodium sulfate and concentrated under vacuum. The residue was purified by silica gel chromatography using 0 to 20% DCM in hexane to furnish 2-bromo-1-(isopropoxymethyl)-3-methyl-benzene (41.5 g, 96%) as a light yellow oil. ¹H NMR (500 MHz, Chloroform-d) δ 7.34 (d, J=8.1 Hz, 1H), 7.20 (t, J=7.5 Hz, 1H), 7.15 (d, J=7.3 Hz, 1H), 4.57 (s, 2H), 3.77-3.72 (m, 1H), 2.42 (s, 3H), 1.26 (d, J=6.1 Hz, 6H). ESI-MS m/z calc. 242.03062, Retention time: 3.84 minutes; LC Method T.

### Step 2: 2-[2-(Isopropoxymethyl)-6-methyl-phenyl]-4,4,5,5-tetramethyl-1,3,2-dioxaborolane

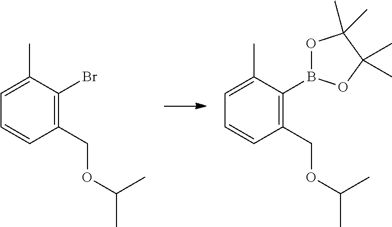 [SMILES: Cc1cccc(COC(C)C)c1Br ; Cc1cccc(COC(C)C)c1B1OC(C)(C)C(C)(C)O1]

A solution of 2-bromo-1-(isopropoxymethyl)-3-methyl-benzene (41.5 g, 136.55 mmol) in anhydrous dioxane (450 mL) was added KOAc (33.5 g, 341.34 mmol). The mixture was degassed with nitrogen for 5 min before adding 4,4,5,5-tetramethyl-2-(4,4,5,5-tetramethyl-1,3,2-dioxaborolan-2-yl)-1,3,2-dioxaborolane (62.4 g, 245.73 mmol) and Pd(dppf)Cl₂ (9.8 g, 13.393 mmol), and then degassed for another 5 min. The tube was then sealed and heated to 100° C. and stirred for 18 h. After the reaction was cooled to room temperature, saturated ammonium chloride (200 mL) was added and extracted with ethyl acetate (3×200 mL). The combined organic extracts washed with brine (250 mL), dried over sodium sulfate, filtered and concentrated. The residue was purified by silica gel chromatography using 0 to 20% EtOAc in hexane to furnish 2-[2-(isopropoxymethyl)-6-methyl-phenyl]-4,4,5,5-tetramethyl-1,3,2-dioxaborolane (30.37 g, 77%) as a light green oil. ESI-MS m/z calc. 290.20532, found 291.3 (M+1)⁺; Retention time: 3.9 minutes; LC Method T.

### Step 3: tert-Butyl N-tert-butoxycarbonyl-N-[4-chloro-6-[2-(isopropoxymethyl)-6-methyl-phenyl]-5-methyl-pyrimidin-2-yl]carbamate

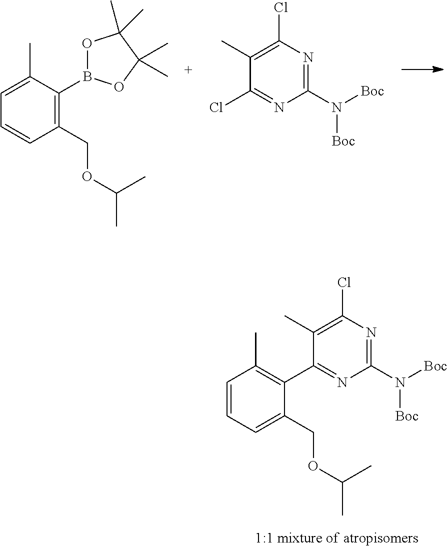 [SMILES: Cc1cccc(COC(C)C)c1B1OC(C)(C)C(C)(C)O1 ; Cc1c(Cl)nc(N(C(=O)OC(C)(C)C)C(=O)OC(C)(C)C)nc1Cl ; Cc1cccc(COC(C)C)c1-c1nc(N(C(=O)OC(C)(C)C)C(=O)OC(C)(C)C)nc(Cl)c1C]

A reaction flask was charged with 2-[2-(isopropoxymethyl)-6-methyl-phenyl]-4,4,5,5-tetramethyl-1,3,2-dioxaborolane (30.37 g, 104.65 mmol), tert-butyl N-tert-butoxycarbonyl-N-(4,6-dichloro-5-methyl-pyrimidin-2-yl)carbamate (44.6 g, 112.02 mmol) and Cs₂CO₃ (85.2 g, 261.50 mmol) in a solvent mixture of DME (585 mL) and water (115 mL). The reaction was purged with argon for 5 min. Pd(dppf)Cl₂ (6.2 g, 8.4734 mmol) was added to reaction mixture. The reaction mixture was purged with argon for another 5 min. The reaction mixture was stirred at 85° C. for 3.5 h. The reaction was cooled to rt and diluted with water (500 mL). The aqueous layer was separated and extracted with EtOAc (3×400 mL). The combined organic layers were washed with brine (500 mL), dried over anhydrous sodium sulfate and concentrated under vacuum. The residue was purified by silica gel chromatography using 0 to 25% ethyl acetate in hexane to furnish tert-butyl N-tert-butoxycarbonyl-N-[4-chloro-6-[2-(isopropoxymethyl)-6-methyl-phenyl]-5-methyl-pyrimidin-2-yl]carbamate (32.8 g, 62%) as a yellow gel. ESI-MS m/z calc. 505.23434, found 506.3 (M+1)⁺; Retention time: 4.29 minutes; LC Method T.

### Step 4: 4-Chloro-6-[2-(isopropoxymethyl)-6-methyl-phenyl]-5-methyl-pyrimidin-2-amine

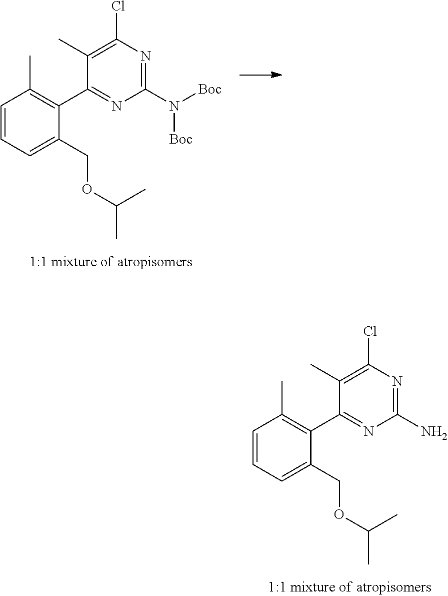 [SMILES: Cc1cccc(COC(C)C)c1-c1nc(N(C(=O)OC(C)(C)C)C(=O)OC(C)(C)C)nc(Cl)c1C ; Cc1cccc(COC(C)C)c1-c1nc(N)nc(Cl)c1C]

To a solution of tert-butyl N-tert-butoxycarbonyl-N-[4-chloro-6-[2-(isopropoxymethyl)-6-methyl-phenyl]-5-methyl-pyrimidin-2-yl]carbamate (32.89 g, 63.046 mmol) in DCM (300 mL) was added a solution of HCl in dioxane (140 mL of 4 M, 560.0 mmol) at 0° C. The reaction was stirred at room temperature overnight. The volatiles were removed under vacuum, and the resulting solid was triturated with diethyl ether (250 mL). The white solid was dissolved with DCM (500 mL), washed with saturated sodium bicarbonate (300 mL), dried over anhydrous sodium sulfate filtered and dried in vacuo to furnish 4-chloro-6-[2-(isopropoxymethyl)-6-methyl-phenyl]-5-methyl-pyrimidin-2-amine (18.929 g, 97%). ¹H NMR (500 MHz, DMSO-d₆) δ 7.32-7.21 (m, 3H), 6.87 (s, 2H), 4.13 (d, J=2.6 Hz, 2H), 3.38-3.33 (m, 1H), 1.98 (s, 3H), 1.78 (s, 3H), 0.99 (d, J=6.1 Hz, 3H), 0.89 (d, J=6.1 Hz, 3H). ESI-MS m/z calc. 305.1295, found 306.4 (M+1)⁺; Retention time: 2.39 minutes; LC Method W.

### Step 5: Methyl 3-[[4-chloro-6-[2-(isopropoxymethyl)-6-methyl-phenyl]-5-methyl-pyrimidin-2-yl]sulfamoyl]benzoate

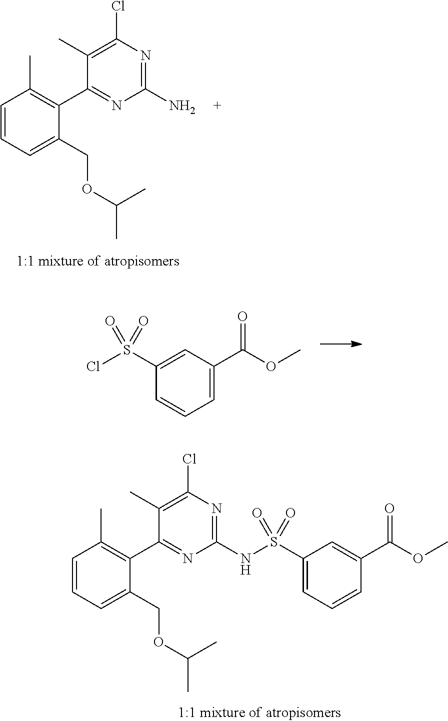

To a solution of 4-chloro-6-[2-(isopropoxymethyl)-6-methyl-phenyl]-5-methyl-pyrimidin-2-amine (1 g, 3.270 mmol) and methyl 3-chlorosulfonylbenzoate (2.33 g, 9.929 mmol) in THF (20 mL) was added a solution of LiHMDS in THF (13.2 mL of 1.0 M, 13.20 mmol) dropwise over 10 min at −78° C. in a dry ice/acetone bath. The reaction mixture was stirred for 3 h at −78° C., and then quenched with 1 N HCl solution. The reaction was warmed to room temperature, diluted with ethyl acetate, and the layers were separated. The aqueous layer was extracted with ethyl acetate (3×). The combined organic layer was washed with brine, dried over sodium sulfate, filtered and concentrated in vacuo. The resulting yellow oil was purified by silica gel chromatography, eluting with a 0-100% gradient of ethyl acetate in hexanes, to give methyl 3-[[4-chloro-6-[2-(isopropoxymethyl)-6-methyl-phenyl]-5-methyl-pyrimidin-2-yl]sulfamoyl]benzoate (1.09 g, 66%) ESI-MS m/z calc. 503.12817, found 504.3 (M+1)⁺; Retention time: 0.74 minutes; LC Method D.

### Step 6: 3-[[4-Chloro-6-[2-(isopropoxymethyl)-6-methyl-phenyl]-5-methyl-pyrimidin-2-yl]sulfamoyl]benzoic acid

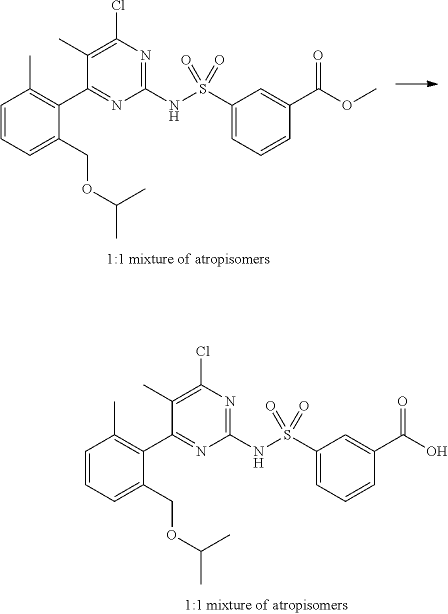 [SMILES: COC(=O)c1cccc(S(=O)(=O)Nc2nc(Cl)c(C)c(-c3c(C)cccc3COC(C)C)n2)c1 ; Cc1cccc(COC(C)C)c1-c1nc(NS(=O)(=O)c2cccc(C(=O)O)c2)nc(Cl)c1C]

Methyl 3-[[4-chloro-6-[2-(isopropoxymethyl)-6-methyl-phenyl]-5-methyl-pyrimidin-2-yl]sulfamoyl]benzoate (1.09 g, 2.163 mmol) was dissolved in THF (15 mL). The reaction was cooled in an ice bath, then aqueous NaOH (12 mL of 1 M, 12.0 mmol) was added. After 5 min the ice bath was removed, and the reaction mixture was stirred vigorously for 3 h. The reaction mixture was then partitioned between 1 N HCl solution and ethyl acetate. The layers were separated and the aqueous layer was extracted with ethyl acetate (3×). The combined organics were washed with brine, dried over sodium sulfate, and concentrated to give a white solid, 3-[[4-chloro-6-[2-(isopropoxymethyl)-6-methyl-phenyl]-5-methyl-pyrimidin-2-yl]sulfamoyl]benzoic acid (1.007 g, 95%) ESI-MS m/z calc. 489.11252, found 490.3 (M+1)⁺; Retention time: 0.65 minutes; LC Method D.

### Step 7: 3-[[4-[(2R)-2-Amino-4,4-dimethyl-pentoxy]-6-[2-(isopropoxymethyl)-6-methyl-phenyl]-5-methyl-pyrimidin-2-yl]sulfamoyl]benzoic acid

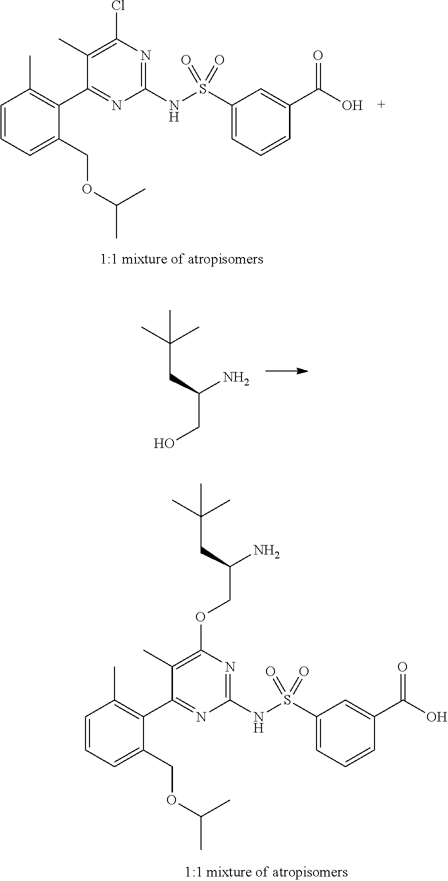

3-[[4-Chloro-6-[2-(isopropoxymethyl)-6-methyl-phenyl]-5-methyl-pyrimidin-2-yl]sulfamoyl]benzoic acid (700 mg, 1.429 mmol) was stirred with (2R)-2-amino-4,4-dimethyl-pentan-1-ol (hydrochloride salt) (255 mg, 1.521 mmol) in anhydrous THF (3.5 mL) at room temperature for 5 min. Then, sodium tert-butoxide (825 mg, 8.585 mmol) was added to the reaction mixture. A mild exotherm was observed and stirring was continued with no external heating for an additional 15 min. The reaction mixture was then partitioned between aqueous 1 N HCl and ethyl acetate. The layers were separated, and the aqueous layer was extracted with ethyl acetate (3×). The combined organics were washed with brine, dried over sodium sulfate and concentrated. The resulting solid was triturated with hexanes/ethyl acetate, then collected by filtration and dried to give a white solid, 3-[[4-[(2R)-2-amino-4,4-dimethyl-pentoxy]-6-[2-(isopropoxymethyl)-6-methyl-phenyl]-5-methyl-pyrimidin-2-yl]sulfamoyl]benzoic acid (hydrochloride salt) (485.7 mg, 55%) ESI-MS m/z calc. 584.26685, found 585.4 (M+1)⁺; Retention time: 0.49 minutes; LC Method D.

### Step 8: (5M,11R)-11-(2,2-dimethylpropyl)-12-{[5-(3,3-dimethylpyrrolidin-1-yl)pyrimidin-2-yl]methyl}-7-methyl-6-{2-methyl-6-[(propan-2-yloxy)methyl]phenyl}-9-oxa-2λ⁶-thia-3,5,12,19-tetraazatricyclo[12.3.1.14,8]nonadeca-1(17),4(19),5,7,14(18),15-hexaene-2,2,13-trione (Compound 220) and (5P,11R)-11-(2,2-dimethylpropyl)-12-{[5-(3,3-dimethylpyrrolidin-1-yl)pyrimidin-2-yl]methyl}-7-methyl-6-{2-methyl-6-[(propan-2-yloxy)methyl]phenyl}-9-oxa-2λ⁶-thia-3,5,12,19-tetraazatricyclo[12.3.1.14,8]nonadeca-1(17),4(19),5,7,14(18),15-hexaene-2,2,13-trione (Compound 221)

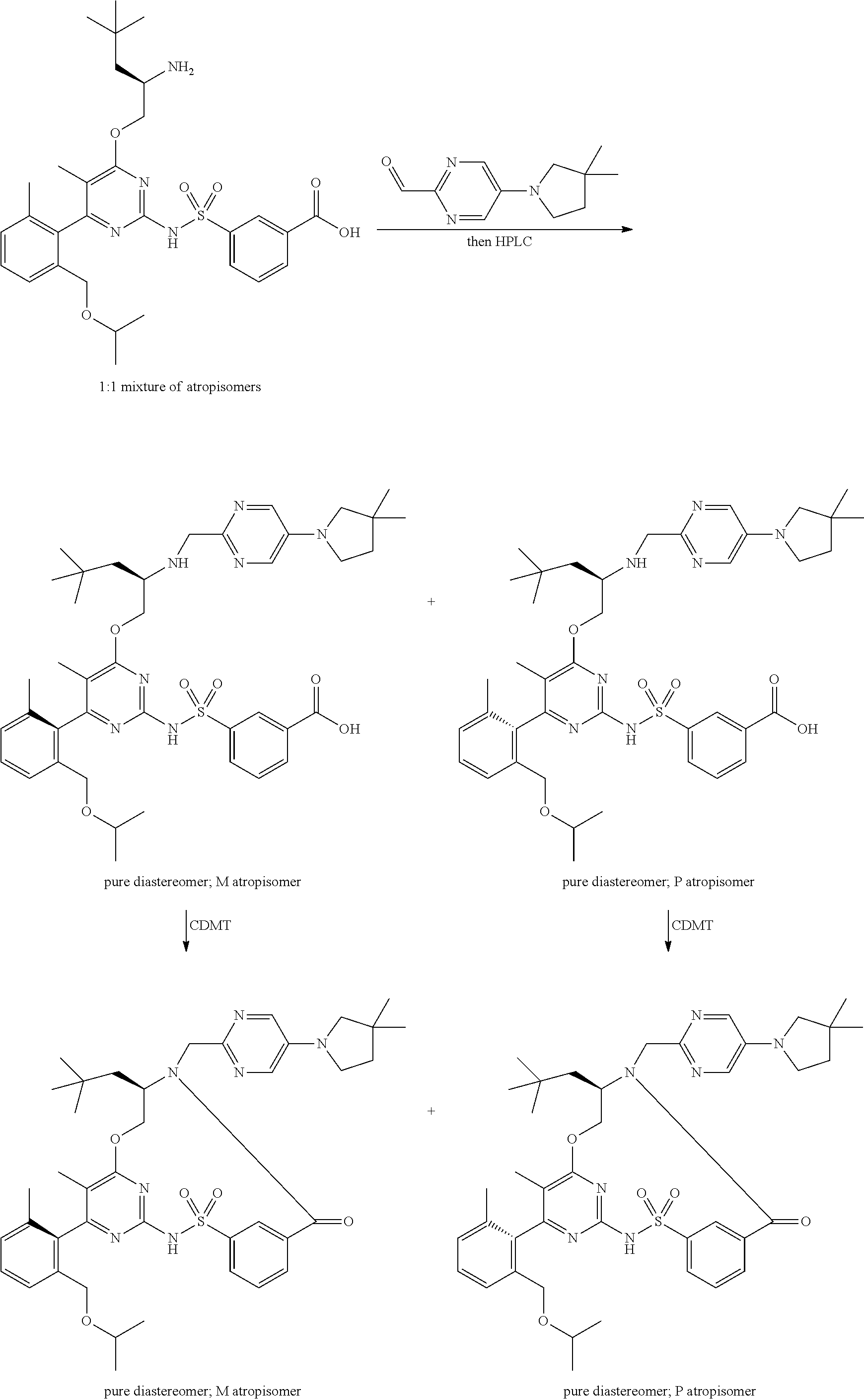

Stage 1: A 4-mL vial was charged under nitrogen with 3-[[4-[(2R)-2-amino-4,4-dimethyl-pentoxy]-6-[2-(isopropoxymethyl)-6-methyl-phenyl]-5-methyl-pyrimidin-2-yl]sulfamoyl]benzoic acid (hydrochloride salt) (100 mg, 0.1610 mmol), 5-(3,3-dimethylpyrrolidin-1-yl)pyrimidine-2-carbaldehyde (37 mg, 0.1803 mmol), and DCM (400 μL). The mixture was stirred at room temperature for 15 min. Sodium triacetoxyborohydride (36 mg, 0.1699 mmol) was added and the mixture was stirred at room temperature for 15 min. More sodium triacetoxyborohydride (109 mg, 0.5143 mmol) was added and the mixture was stirred at room temperature for 1 h. The reaction was quenched with aqueous 1 N HCl. Methanol and DMSO were added. After filtration, purification by reverse-phase HPLC (1-99% acetonitrile/5 mM HCl) provided separately 3-[[4-[(2R)-2-[[5-(3,3-dimethylpyrrolidin-1-yl)pyrimidin-2-yl]methylamino]-4,4-dimethyl-pentoxy]-6-[2-(isopropoxymethyl)-6-methyl-phenyl]-5-methyl-pyrimidin-2-yl]sulfamoyl]benzoic acid (hydrochloride salt) (18 mg, 28%) (diastereomer 1, more polar peak, first to elute). ESI-MS m/z calc. 773.39343, found 774.7 (M+1)⁺; Retention time: 0.62 minutes; LC Method D. This also gave 3-[[4-[(2R)-2-[[5-(3,3-dimethylpyrrolidin-1-yl)pyrimidin-2-yl]methylamino]-4,4-dimethyl-pentoxy]-6-[2-(isopropoxymethyl)-6-methyl-phenyl]-5-methyl-pyrimidin-2-yl]sulfamoyl]benzoic acid (hydrochloride salt) (23.7 mg, 36%) (diastereomer 2, less polar peak, second to elute). ESI-MS m/z calc. 773.39343, found 774.7 (M+1)⁺; Retention time: 0.63 minutes; LC Method D.

Stage 2 (from diastereomer 1): 3-[[4-[(2R)-2-[[5-(3,3-dimethylpyrrolidin-1-yl)pyrimidin-2-yl]methylamino]-4,4-dimethyl-pentoxy]-6-[2-(isopropoxymethyl)-6-methyl-phenyl]-5-methyl-pyrimidin-2-yl]sulfamoyl]benzoic acid (hydrochloride salt) (18 mg, 28%) (diastereomer 1 from Stage 1) was combined with CDMT (6 mg, 0.03417 mmol) in DMF (2 mL) and cooled to 0° C. in an ice bath. 4-Methylmorpholine (20 μL, 0.1819 mmol) was added, and after 1 h the ice bath was removed. The reaction mixture was allowed to stir an additional 4 h at room temperature, then was filtered and purified by reverse-phase preparative HPLC (1-99% MeCN in water, HCl modifier) to give the more polar, first-to-elute diastereomer, (5M,11R)-11-(2,2-dimethylpropyl)-12-{[5-(3,3-dimethylpyrrolidin-1-yl)pyrimidin-2-yl]methyl}-7-methyl-6-{2-methyl-6-[(propan-2-yloxy)methyl]phenyl}-9-oxa-2λ⁶-thia-3,5,12,19-tetraazatricyclo[12.3.1.14,8]nonadeca-1(17),4(19),5,7,14(18),15-hexaene-2,2,13-trione (8.4 mg, 13%). ¹H NMR (400 MHz, DMSO-d₆) δ 12.78 (s, 1H), 8.70 (s, 1H), 8.08 (s, 2H), 7.86 (d, J=6.7 Hz, 1H), 7.63 (s, 2H), 7.37 (t, J=7.5 Hz, 1H), 7.29 (dd, J=9.8, 7.3 Hz, 2H), 5.45 (d, J=9.3 Hz, 1H), 4.87 (d, J=16.2 Hz, 1H), 4.54 (d, J=16.3 Hz, 1H), 4.06 (t, J=11.0 Hz, 2H), 3.90 (d, J=11.4 Hz, 2H), 3.39 (t, J=7.0 Hz, 2H), 3.19-3.12 (m, 1H), 3.08 (s, 2H), 2.05 (s, 3H), 1.94-1.85 (m, 1H), 1.77 (t, J=6.9 Hz, 2H), 1.58 (s, 3H), 1.37 (d, J=14.9 Hz, 1H), 1.10 (s, 6H), 0.81 (d, J=6.0 Hz, 3H), 0.70 (d, J=6.1 Hz, 3H), 0.65 (s, 9H). ESI-MS m/z calc. 755.3829, found 756.7 (M+1)⁺; Retention time: 2.05 minutes; LC Method A.

Stage 2 (from diastereomer 2): 3-[[4-[(2R)-2-[[5-(3,3-dimethylpyrrolidin-1-yl)pyrimidin-2-yl]methylamino]-4,4-dimethyl-pentoxy]-6-[2-(isopropoxymethyl)-6-methyl-phenyl]-5-methyl-pyrimidin-2-yl]sulfamoyl]benzoic acid (hydrochloride salt) (23.7 mg, 36%) (diastereomer 2 from Stage 1) was combined with CDMT (6 mg, 0.03417 mmol) in DMF (2 mL) and cooled to 0° C. in an ice bath. 4-Methylmorpholine (20 μL, 0.1819 mmol) was added, and after 1 h the ice bath was removed. The reaction mixture was allowed to stir an additional 4.5 h at room temperature, then was filtered and purified by reverse-phase preparative HPLC (1-99% MeCN in water, HCl modifier) to give the less polar, second-to-elute diastereomer, (5P,11R)-11-(2,2-dimethylpropyl)-12-{[5-(3,3-dimethylpyrrolidin-1-yl)pyrimidin-2-yl]methyl}-7-methyl-6-{2-methyl-6-[(propan-2-yloxy)methyl]phenyl}-9-oxa-2λ⁶-thia-3,5,12,19-tetraazatricyclo[12.3.1.14,8]nonadeca-1(17),4(19),5,7,14(18),15-hexaene-2,2,13-trione (6.8 mg, 110%). ¹H NM/R (400 MHz, DMSO-d₆) δ 12.95 (s, 1H), 8.70 (s, 1H), 8.08 (s, 2H), 7.93 (s, 1H), 7.66 (s, 2H), 7.34 (q, J=7.4 Hz, 2H), 7.24 (d, J=6.5 Hz, 1H), 5.41 (s, 1H), 4.85 (d, J=16.2 Hz, 1H), 4.54 (d, J=16.2 Hz, 1H), 4.18 (d, J=12.2 Hz, 3H), 4.04 (d, J=9.3 Hz, 1H), 3.41 (s, 1H), 3.40 (s, 2H), 3.08 (s, 2H), 1.80 (s, 3H), 1.77 (t, J=7.0 Hz, 3H), 1.60 (s, 3H), 1.37 (d, J=14.7 Hz, 1H), 1.10 (s, 6H), 0.99 (m, 6H), 0.55 (s, 9H). ESI-MS m/z calc. 755.3829, found 756.7 (M+1)⁺; Retention time: 2.08 minutes; LC Method A.

### Example 79: Characterization of Compounds 222-320

The compounds in the following tables were prepared in a manner analogous to that described above using commercially available reagents and intermediates described herein

TABLE 17

| Compound | | LCMS | Calc. | | LCMS |
|---|---|---|---|---|---|
| Number | Structure | Rt (min) | Mass | M + 1 | Method |
| 222 | 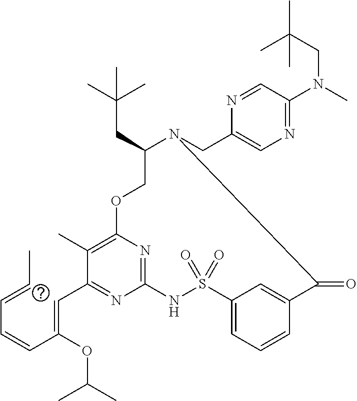 [SMILES: C/C=C/C=C\C(=C\c1nc2nc(c1C)OC[C@@H](CC(C)(C)C)N(Cc1cnc(N(C)CC(C)(C)C)cn1)C(=O)c1cccc(c1)S(=O)(=O)N2)OC(C)C] | 2.97 | 743.383 | 744.7 | W |
| 223 | 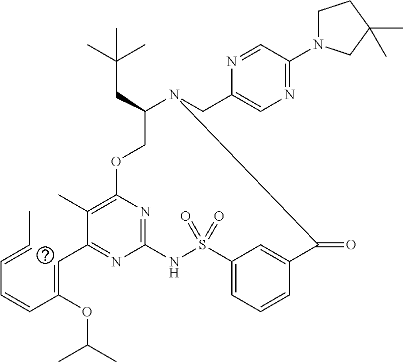 [SMILES: C/C=C/C=C\C(=C\c1nc2nc(c1C)OC[C@@H](CC(C)(C)C)N(Cc1cnc(N3CCC(C)(C)C3)cn1)C(=O)c1cccc(c1)S(=O)(=O)N2)OC(C)C] | 2.99 | 741.367 | 742.6 | W |
| 224 | 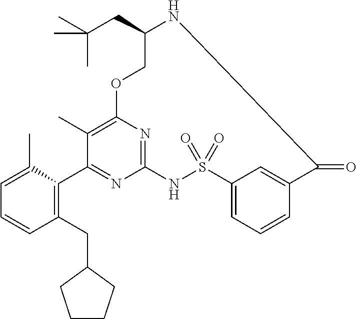 | 2.84 | 576.277 | 577.6 | W |
| 225 | 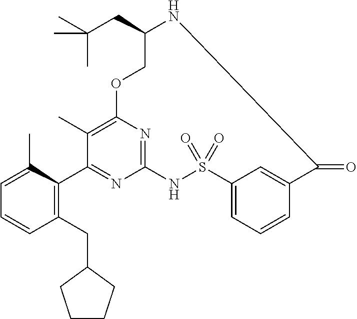 | 2.81 | 576.277 | 577.6 | W |
| 226 | 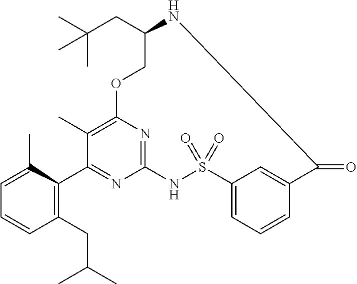 | 2.61 | 550.261 | 551.5 | W |
| 227 | 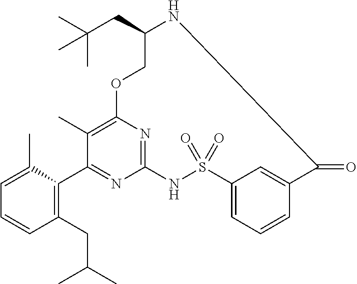 | 2.64 | 550.261 | 551.5 | W |
| 228 | 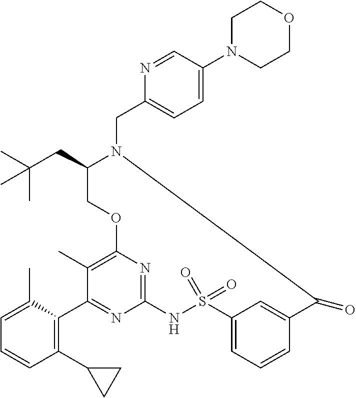 | 2.14 | 710.325 | 711.7 | W |
| 229 | 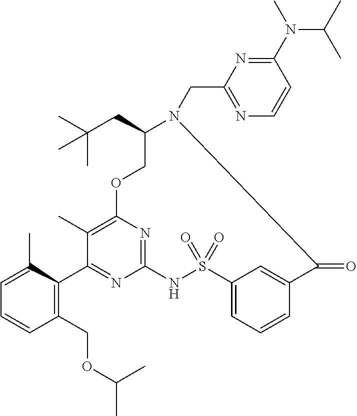 | 1.43 | 729.367 | 730.7 | A |
| 230 | 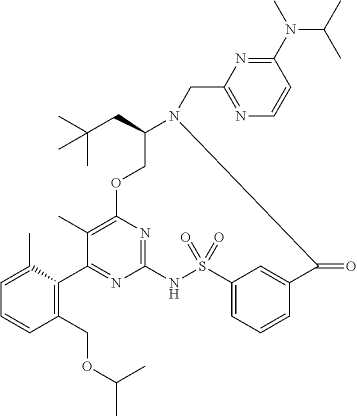 | 1.45 | 729.367 | 730.7 | A |
| 231 | 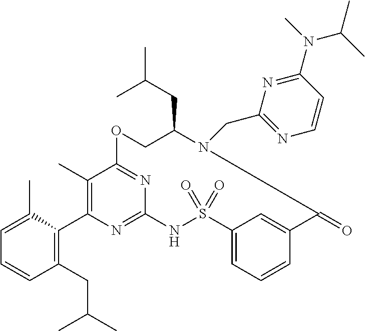 | 3.03 | 699.357 | 700.4 | Y |
| 232 | 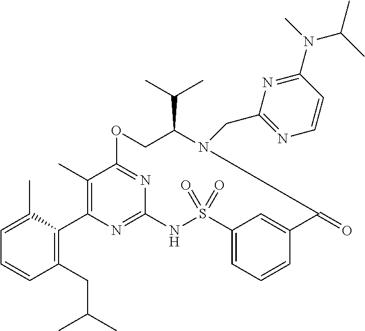 | 2.91 | 685.341 | 686.4 | Y |
| 233 | 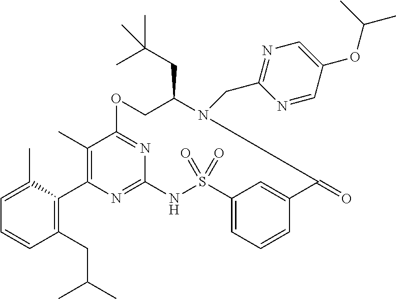 | 4.97 | 700.341 | 701.4 | Y |
| 234 | 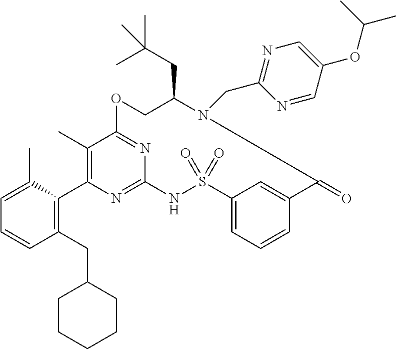 | 5.37 | 740.372 | | Y |
| 235 | 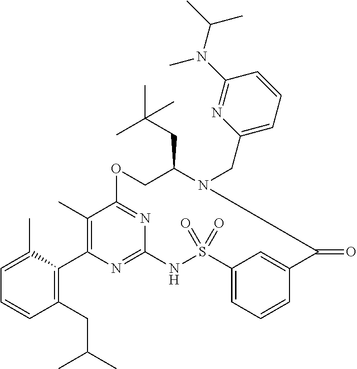 | 4.15 | 712.377 | 713.4 | 1D |
| 219 | 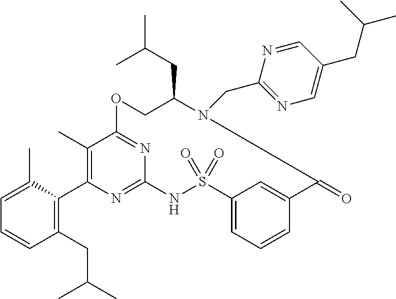 | 5.06 | 684.346 | 685.3 | Y |
| 236 | 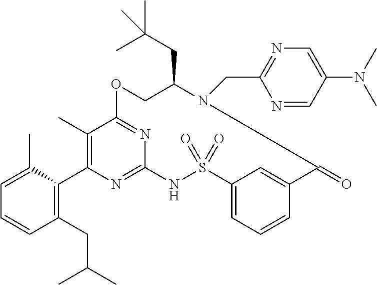 | 4.75 | 685.341 | 686.3 | Y |
| 237 | 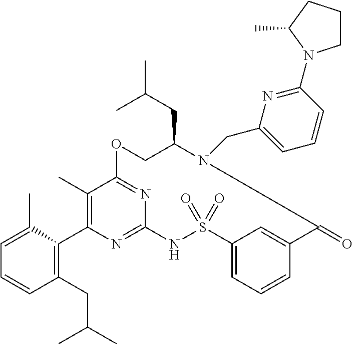 | 3.8 | 710.361 | 711.4 | 1D |
| 238 | 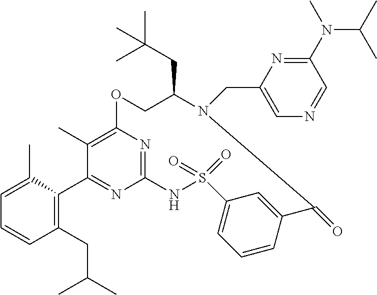 | 3.75 | 713.372 | 714.4 | 1D |
| 239 | 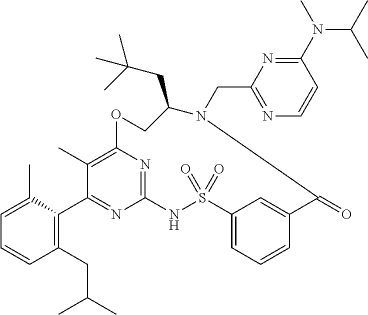 | 3.08 | 713.372 | 714.4 | Y |
| 240 | 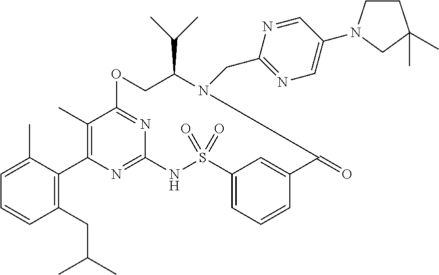 [SMILES: Cc1cccc(CC(C)C)c1-c1nc2nc(c1C)OC[C@@H](C(C)C)N(Cc1ncc(N3CCC(C)(C)C3)cn1)C(=O)c1cccc(c1)S(=O)(=O)N2] | 5.07 | 711.357 | | Y |
| 241 | 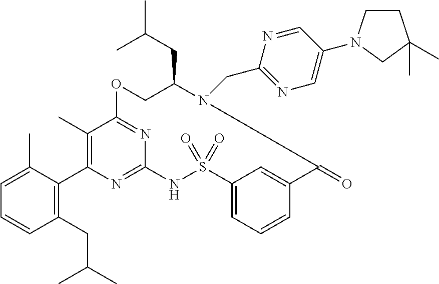 [SMILES: Cc1cccc(CC(C)C)c1-c1nc2nc(c1C)OC[C@@H](CC(C)C)N(Cc1ncc(N3CCC(C)(C)C3)cn1)C(=O)c1cccc(c1)S(=O)(=O)N2] | 3.84 | 725.372 | 726.3 | 1E |
| 242 | 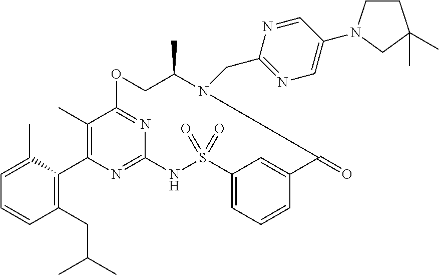 | 4.79 | 683.325 | 684.4 | Y |
| 243 | 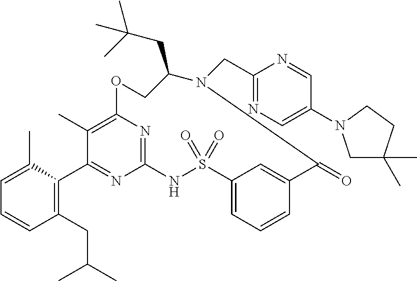 | 3.97 | 739.388 | 740.4 | 1D |
| 244 | 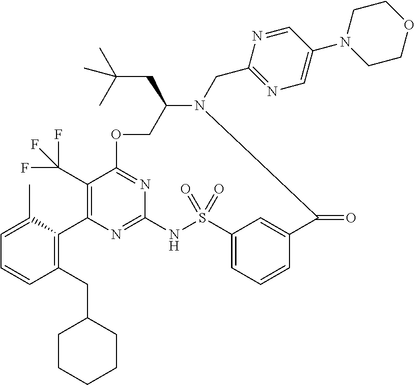 | 3.3 | 821.355 | 822.4 | 1D |
| 245 | 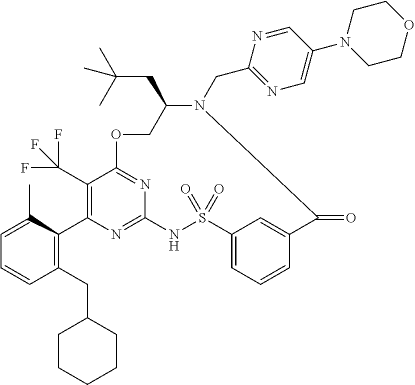 | 3.3 | 821.355 | 822.4 | 1D |
| 246 | 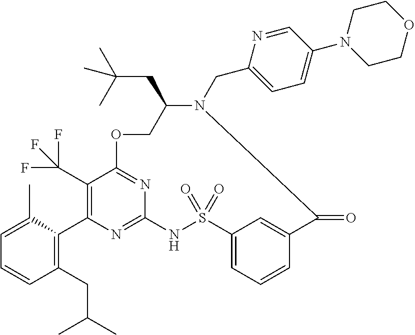 | 3.18 | 780.328 | 781.3 | 1D |
| 247 | 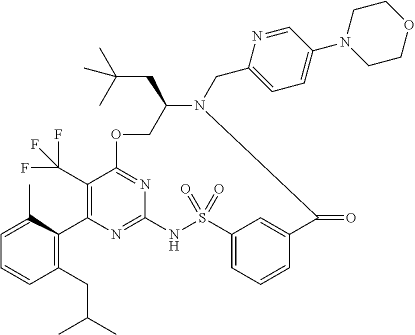 | 3.18 | 780.328 | 781.3 | 1D |
| 248 | 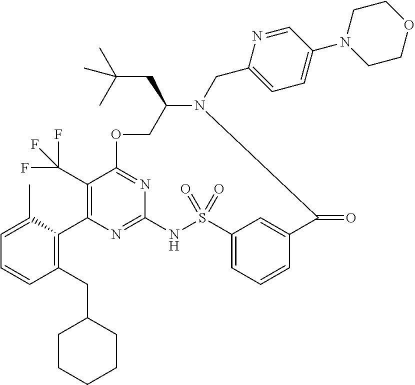 | 3.34 | 820.359 | 821.4 | 1D |
| 249 | 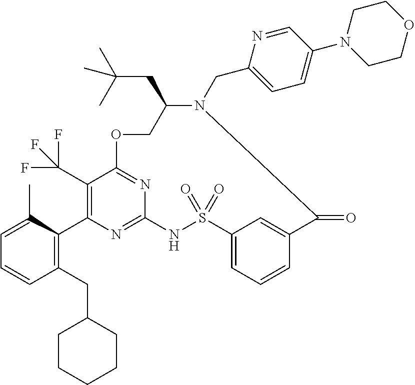 | 3.36 | 820.359 | 821.4 | 1D |
| 250 | 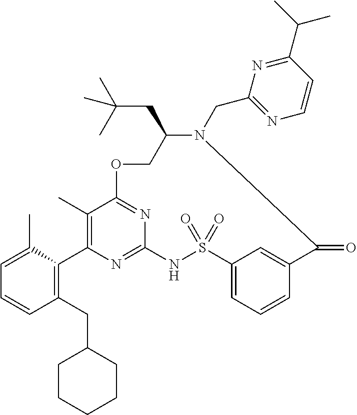 | 4.01 | 724.377 | | 1E |
| 251 | 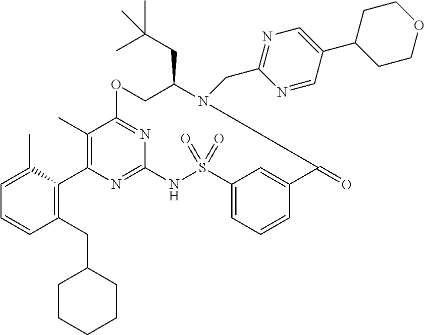 | 5.03 | 766.388 | | Y |
| 252 | 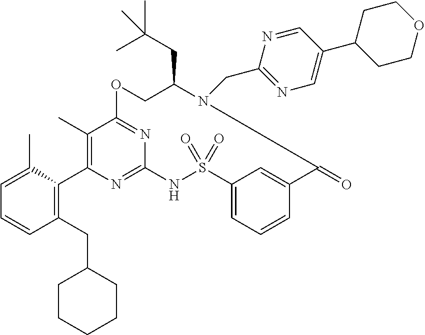 | 5.19 | 766.388 | | Y |
| 253 | 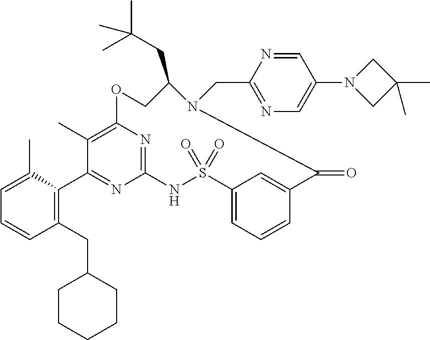 | 4.1 | 765.404 | 766.5 | 1D |
| 254 | 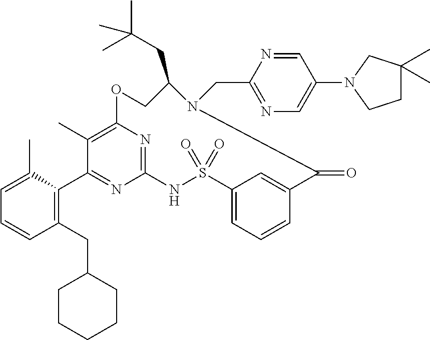 | 4.2 | 779.419 | 780.4 | 1D |
| 255 | 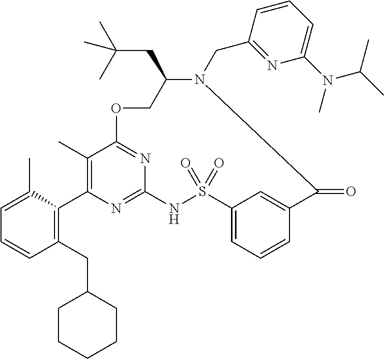 | 3.22 | 752.408 | 753.66 | 1D |
| 256 | 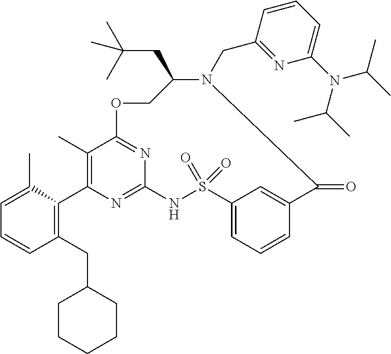 | 4.91 | 780.44 | 781.5 | 1D |
| 257 | 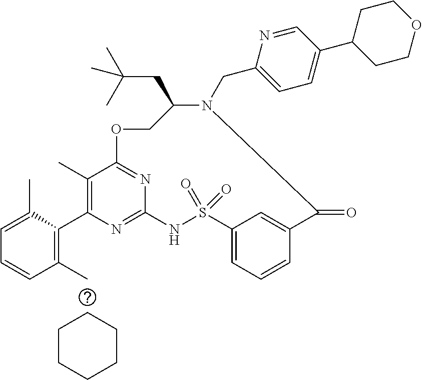 | 5.1 | 765.392 | | Y |
| 258 | 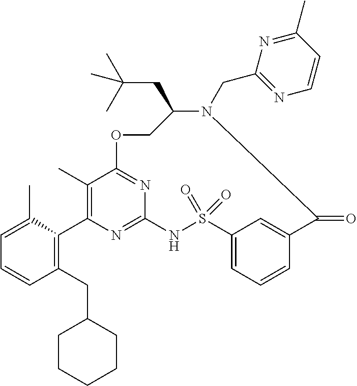 | 5.1 | 696.346 | 697.3 | Y |
| 259 | 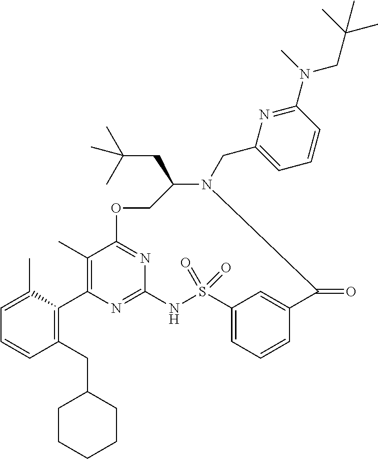 | 4.85 | 780.44 | 781.4 | 1D |
| 260 | 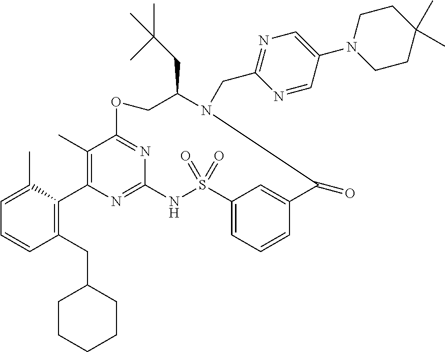 | 4.61 | 793.435 | 794.2 | 1E |
| 261 | 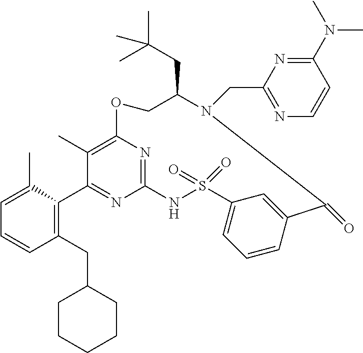 | 3.23 | 725.372 | 726.4 | Y |
| 262 | 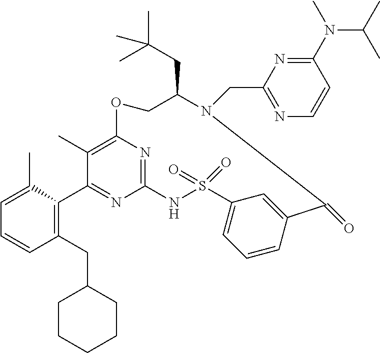 | 3.36 | 753.404 | 754.4 | Y |
| 263 | 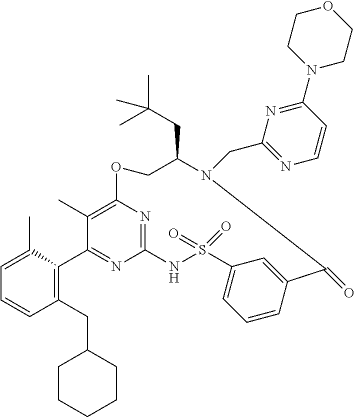 | 3.51 | 767.383 | 768.4 | Y |
| 264 | 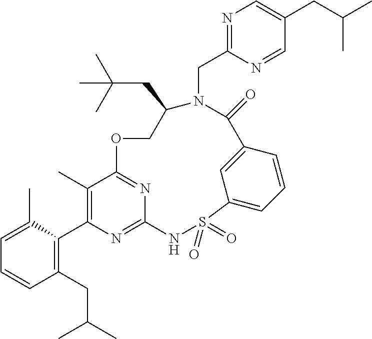 | 2.28 | 698.361 | 699.5 | A |
| 265 | 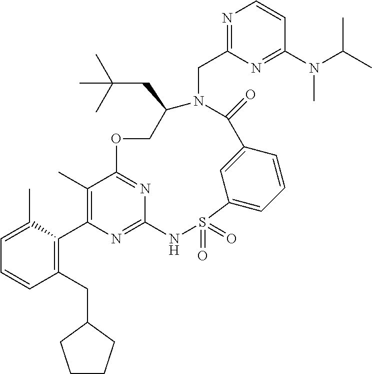 | 1.75 | 739.388 | 740.5 | A |
| 266 | 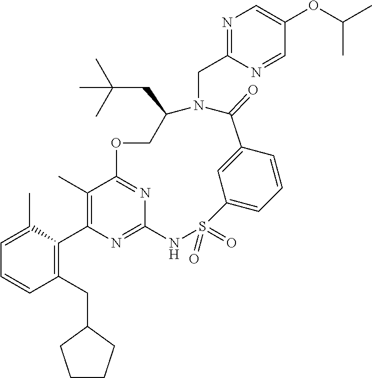 | 2.4 | 726.356 | 727.5 | A |
| 267 | 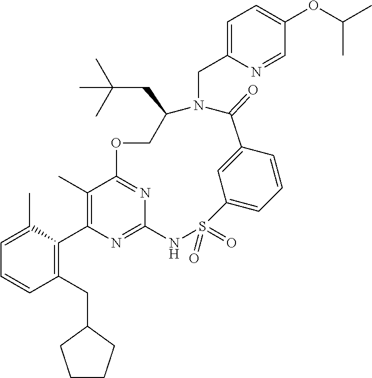 | 2.14 | 725.361 | 726.5 | A |
| 268 | 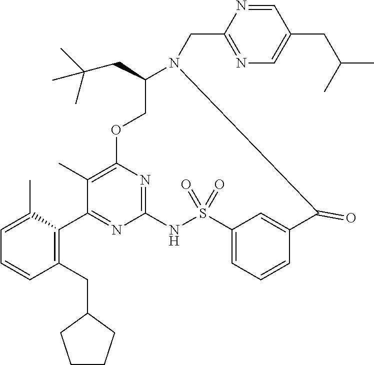 | 2.37 | 724.377 | 725.82 | A |
| 269 | 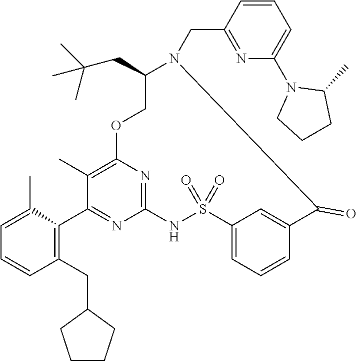 | 1.74 | 750.393 | 751.84 | A |
| 270 | 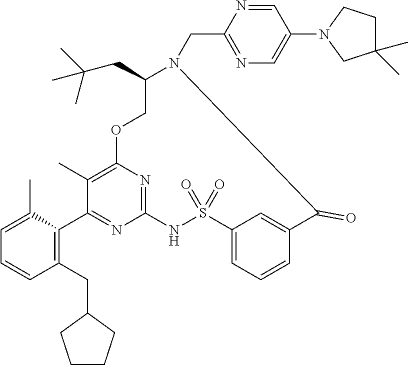 | 2.33 | 765.404 | 766.8 | A |
| 271 | 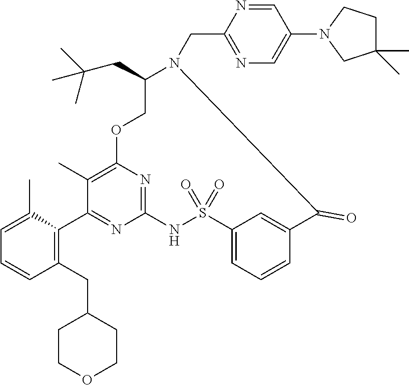 | 2.04 | 781.399 | 782.92 | A |
| 272 | 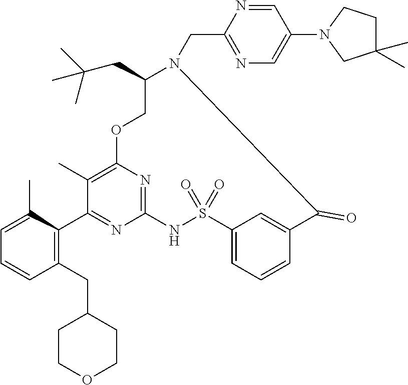 | 2.03 | 781.399 | 782.97 | A |
| 273 | 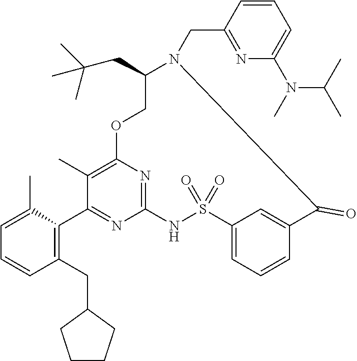 | 1.78 | 738.393 | 739.74 | A |
| 274 | 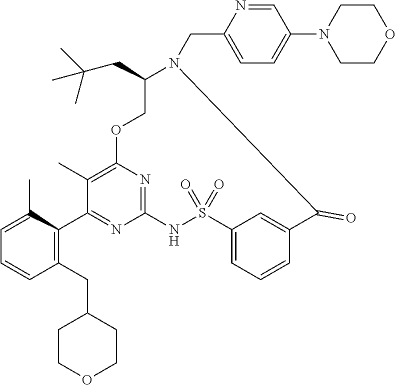 | 1.41 | 768.367 | 769.72 | A |
| 275 | 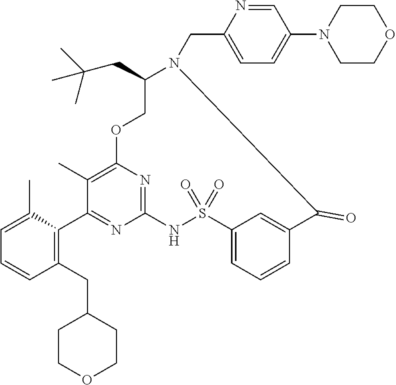 | 1.46 | 768.367 | 769.77 | A |
| 276 | 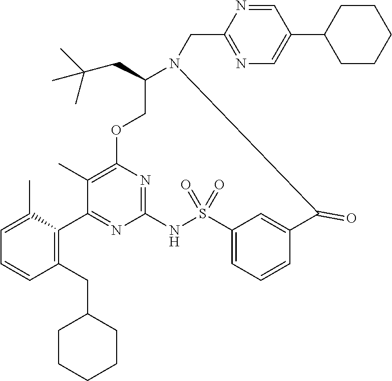 | 2.57 | 764.408 | 765.48 | A |
| 277 | 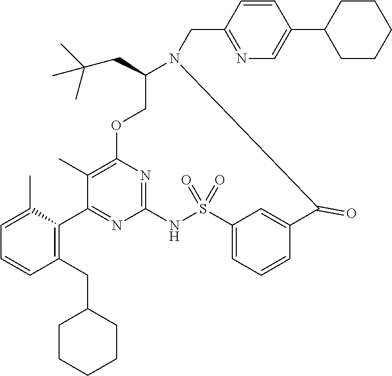 | 2.17 | 763.413 | 764.49 | A |
| 278 | 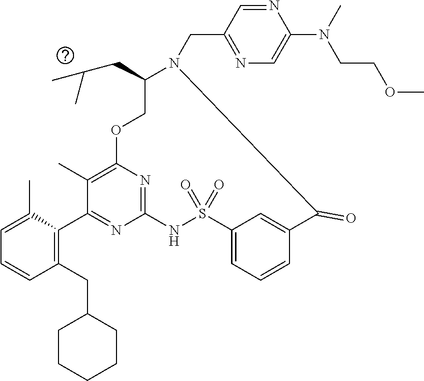 | 2.29 | 769.399 | 770.47 | A |
| 279 | 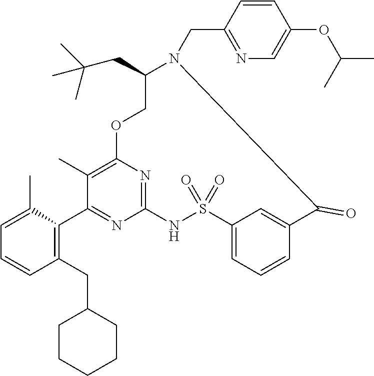 | 2.2 | 739.377 | 740.47 | A |
| 280 | 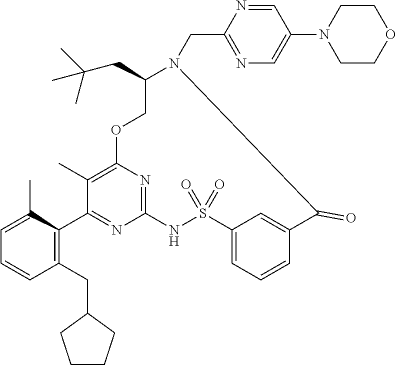 | 2.08 | 753.367 | 754.45 | A |
| 281 | 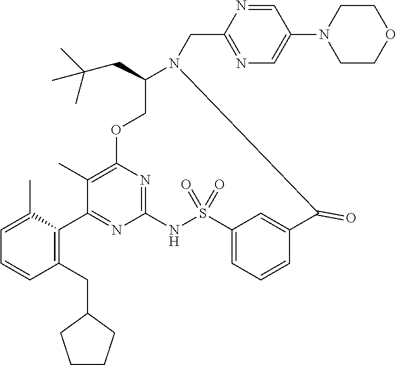 | 2.11 | 753.367 | 754.41 | A |
| 282 | 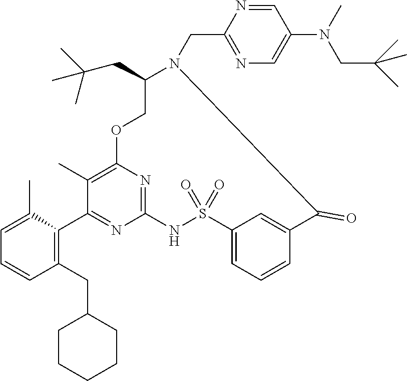 | 1.22 | 781.435 | 782.45 | A (50- 99% gradient) |
| 283 | 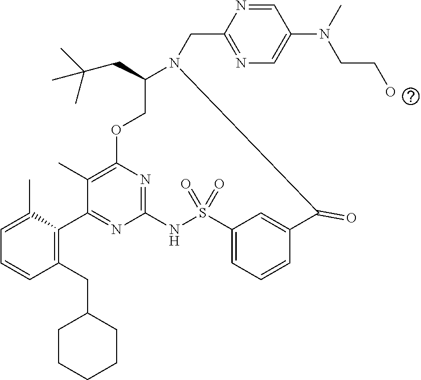 | 2.2 | 769.399 | 770.4 | A |
| 284 | 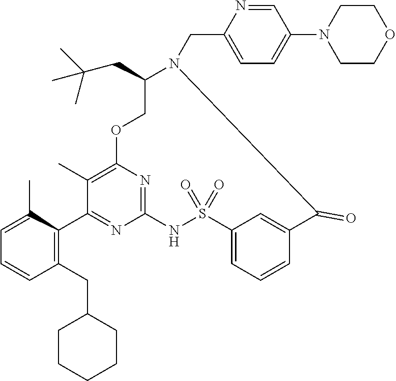 | 1.73 | 752.372 | 753.6 | A |
| 285 | 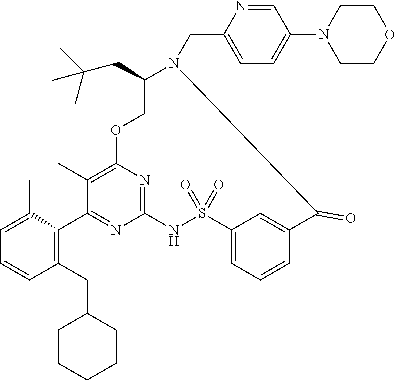 | 1.75 | 752.372 | 753.38 | A |
| 286 | 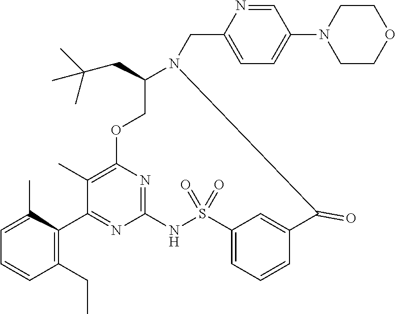 | 2.07 | 698.325 | 699.33 | I |
| 287 | 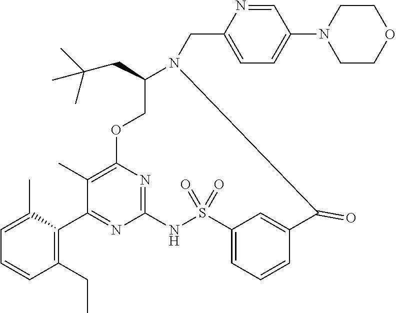 | 2.08 | 698.325 | 699.37 | I |
| 288 | 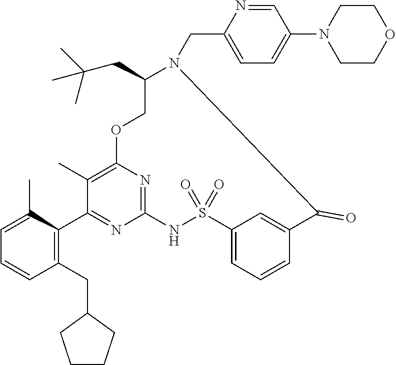 | 1.7 | 752.372 | 753.38 | A |
| 289 | 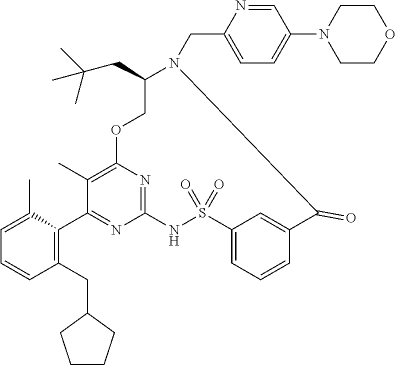 | 1.73 | 752.372 | 753.38 | A |
| 290 | 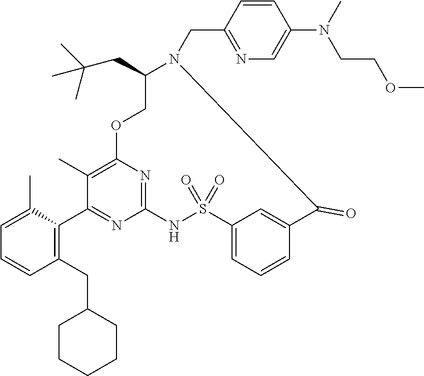 | 1.77 | 768.403 | 769.44 | A |
| 291 | 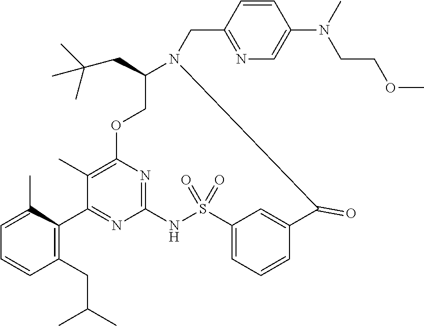 | 1.61 | 728.372 | 729.58 | A |
| 292 | 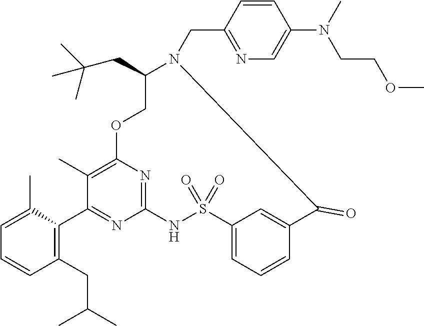 | 1.62 | 728.372 | 729.4 | A |
| 293 | 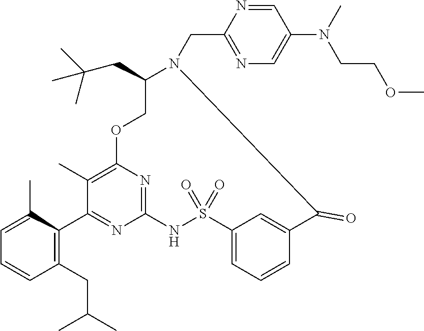 | 1.98 | 729.367 | 730.68 | A |
| 294 | 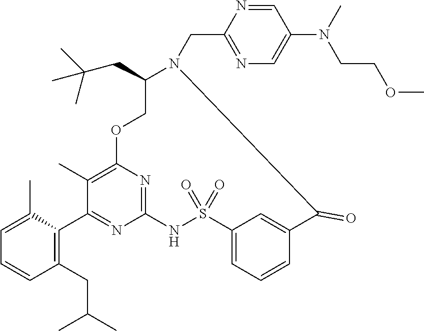 | 2.02 | 729.367 | 730.46 | A |
| 295 | 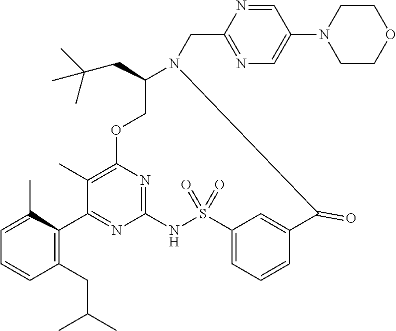 | 1.99 | 727.352 | 728.41 | A |
| 296 | 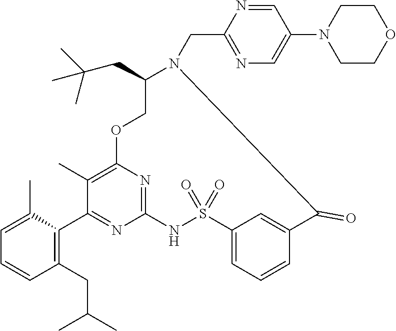 | 2.01 | 727.352 | 728.56 | A |
| 297 | 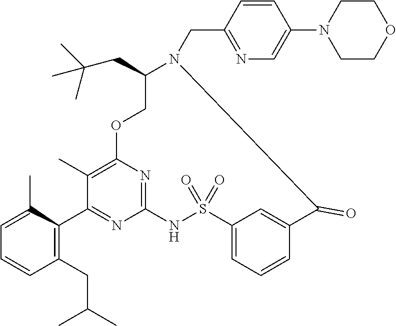 | 1.62 | 726.356 | 727.46 | A |
| 298 | 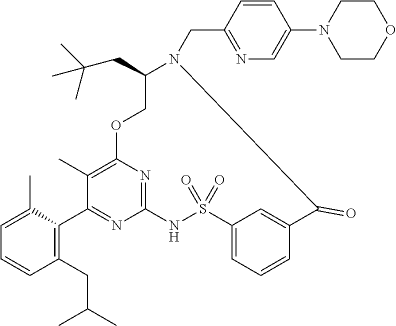 | 1.63 | 726.356 | 727.42 | A |
| 299 | 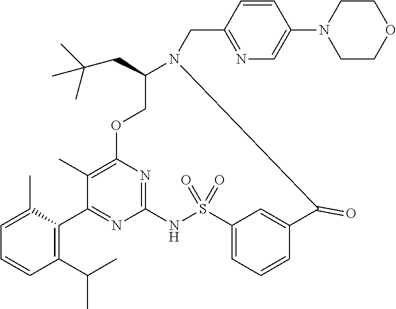 | 1.565 | 712.341 | 713.39 | A |
| 300 | 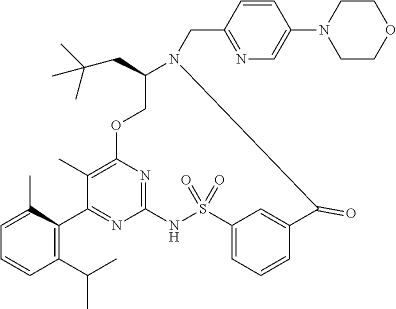 | 1.55 | 712.341 | 713.41 | A |
| 301 | 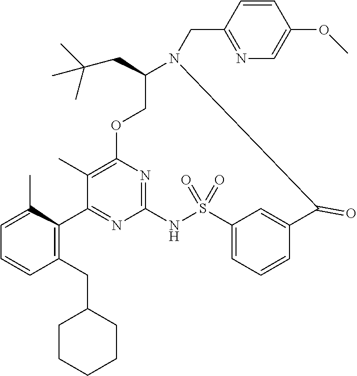 | 2.08 | 711.345 | 712.64 | A |
| 302 | 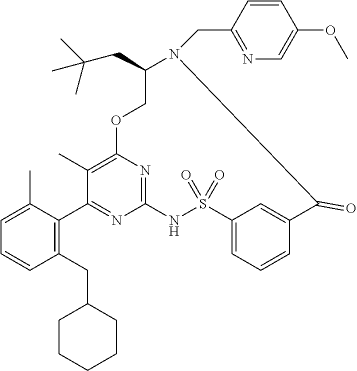 [SMILES: COc1ccc(CN2C(=O)c3cccc(c3)S(=O)(=O)Nc3nc(c(C)c(-c4c(C)cccc4CC4CCCCC4)n3)OC[C@H]2CC(C)(C)C)nc1] | 2.11 | 711.345 | 712.46 | A |
| 303 | 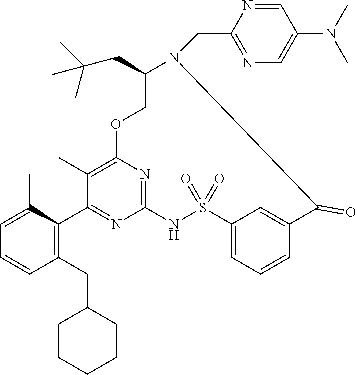 | 2.18 | 725.372 | 726.47 | A |
| 304 | 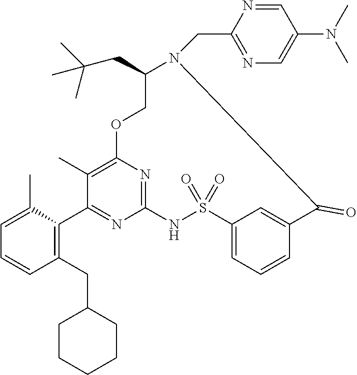 | 2.22 | 725.372 | 726.43 | A |
| 305 | 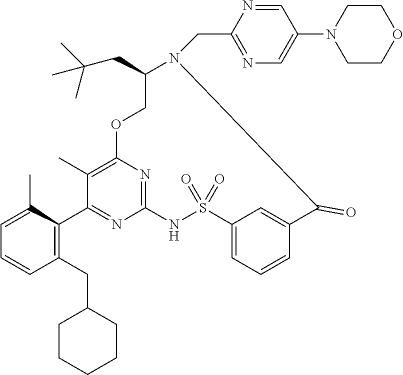 | 2.19 | 767.383 | 768.55 | A |
| 306 | 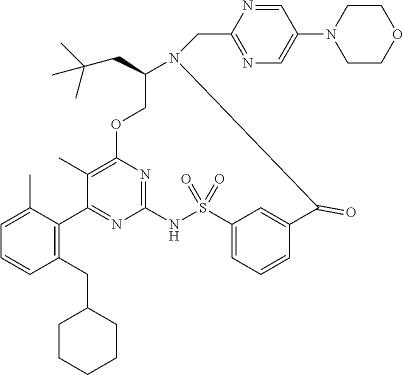 [SMILES: Cc1cccc(CC2CCCCC2)c1-c1nc2nc(c1C)OC[C@@H](CC(C)(C)C)N(Cc1ncc(N3CCOCC3)cn1)C(=O)c1cccc(c1)S(=O)(=O)N2] | 2.22 | 767.383 | 768.55 | A |
| 307 | 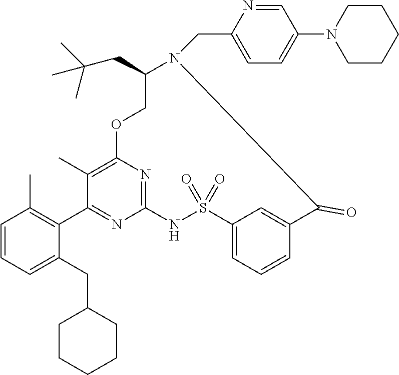 [SMILES: Cc1cccc(CC2CCCCC2)c1-c1nc2nc(c1C)OC[C@@H](CC(C)(C)C)N(Cc1ccc(N3CCCCC3)cn1)C(=O)c1cccc(c1)S(=O)(=O)N2] | 1.8 | 764.408 | 765.65 | A |
| 308 | 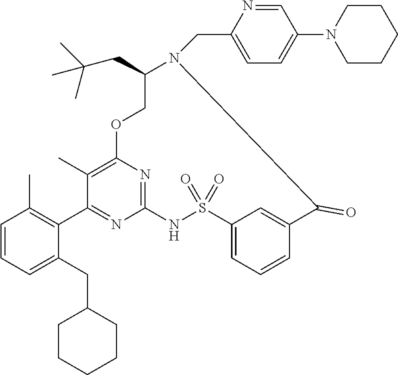 [SMILES: Cc1cccc(CC2CCCCC2)c1-c1nc2nc(c1C)OC[C@@H](CC(C)(C)C)N(Cc1ccc(N3CCCCC3)cn1)C(=O)c1cccc(c1)S(=O)(=O)N2] | 1.83 | 764.408 | 765.65 | A |
| 309 | 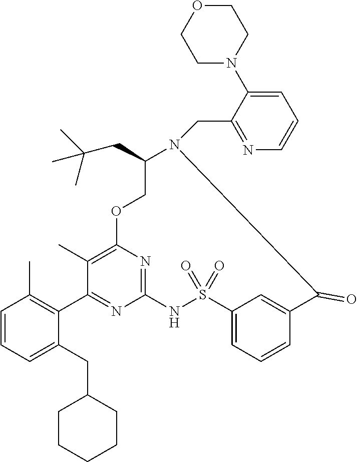 [SMILES: Cc1cccc(CC2CCCCC2)c1-c1nc2nc(c1C)OC[C@@H](CC(C)(C)C)N(Cc1ncccc1N1CCOCC1)C(=O)c1cccc(c1)S(=O)(=O)N2] | 2 | 766.388 | 767.41 | A |
| 310 | 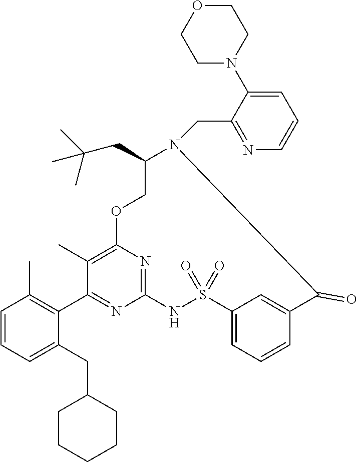 [SMILES: Cc1cccc(CC2CCCCC2)c1-c1nc2nc(c1C)OC[C@@H](CC(C)(C)C)N(Cc1ncccc1N1CCOCC1)C(=O)c1cccc(c1)S(=O)(=O)N2] | 2.03 | 766.388 | 767.41 | A |
| 311 | 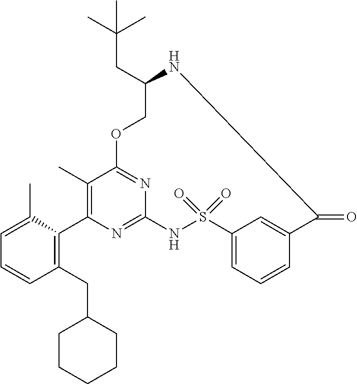 | 2.05 | 590.293 | 591.335 | A |
| 312 | 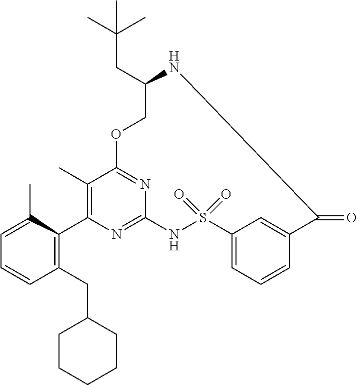 | 2.03 | 590.293 | 591.44 | A |
| 313 | 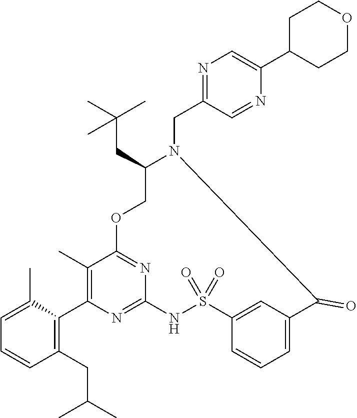 | 2.07 | 726.356 | 727.7 | A |
| 314 | 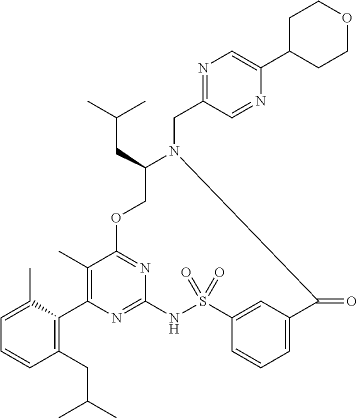 | 2.02 | 712.341 | 713.6 | A |
| 315 | 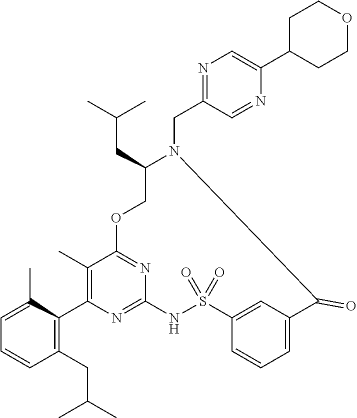 | 1.99 | 712.341 | 713.7 | A |
| 316 | 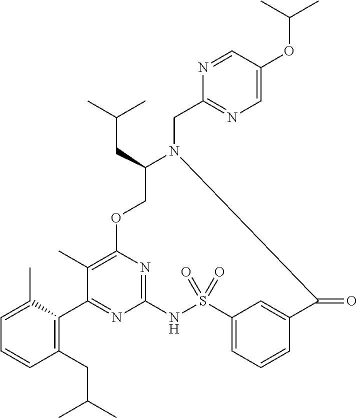 | 2.14 | 686.325 | 687.7 | A |
| 317 | 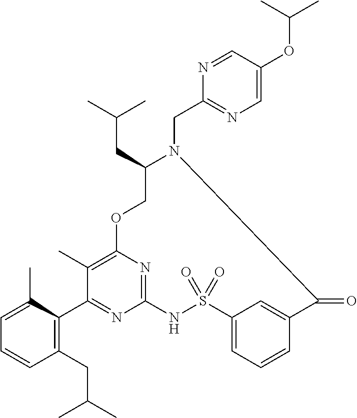 | 2.12 | 686.325 | 687.7 | A |
| 318 | 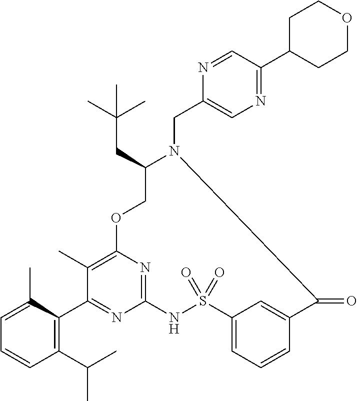 | 1.96 | 712.341 | 713.7 | A |
| 319 | 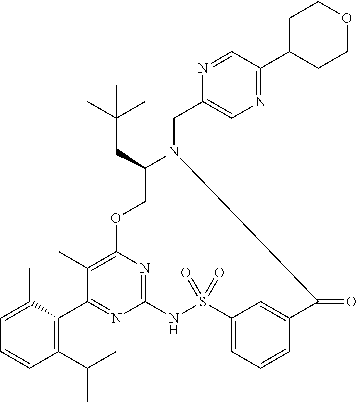 | 2.02 | 712.341 | 713.5 | A |
| 320 | 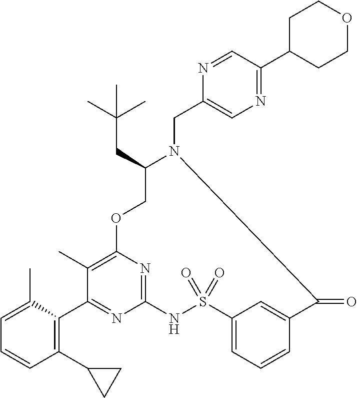 | 1.9 | 710.325 | 711.7 | A |

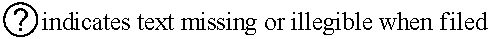

TABLE 18

| Compound | |
|---|---|
| Number | NMR |
| 222 | ¹H NMR (500 MHz, DMSO-d₆) δ 12.84 (s, 1H), 8.65 (s, 1H), 8.07 (s, 1H), |
| | 7.89 (s, 1H), 7.83 (s, 1H), 7.65 (s, 2H), 7.35-7.27 (m, 1H), 6.93 (d, J = 8.4 |
| | Hz, 1H), 6.88 (d, J = 7.6 Hz, 1H), 5.43-5.35 (m, 1H), 4.73 (d, J = 15.7 |
| | Hz, 1H), 4.49 (p, J = 6.0 Hz, 1H), 4.43 (d, J = 15.7 Hz, 1H), 4.32-4.25 |
| | (m, 1H), 4.10-4.00 (m, 1H), 3.51-3.40 (m, 2H), 3.15 (s, 3H), 2.03 (s, 3H), |
| | 1.87-1.78 (m, 1H), 1.60 (s, 3H), 1.35 (d, J = 15.1 Hz, 1H), 1.00-0.94 (m, |
| | 6H), 0.91 (s, 9H), 0.57 (s, 9H). |
| 223 | ¹H NMR (500 MHz, DMSO-d₆) δ 12.86 (s, 1H), 8.68 (s, 1H), 8.06 (s, 2H), |
| | 7.89 (s, 1H), 7.63 (s, 2H), 7.31 (s, 1H), 6.91 (dd, J = 21.0, 8.0 Hz, 2H), 5.39 |
| | (d, J = 9.4 Hz, 1H), 4.85 (d, J = 16.1 Hz, 1H), 4.56-4.46 (m, 2H), 4.14- |
| | 3.97 (m, 2H), 3.37 (m, 1H), 3.07 (s, 2H), 2.03 (s, 3H), 1.85 (dd, J = 15.2, |
| | 9.2 Hz, 1H), 1.76 (t, J = 7.0 Hz, 3H), 1.59 (s, 3H), 1.33 (d, J = 15.2 Hz, |
| | 1H), 1.09 (d, J = 1.2 Hz, 6H), 0.99 (dd, J = 6.2, 2.5 Hz, 6H), 0.58 (s, 9H). |
| 224 | ¹H NMR (500 MHz, DMSO-d₆) δ 13.03 (s, 1H), 8.52 (s, 1H), 7.89 (m, 2H), |
| | 7.64 (m, 2H), 7.38-7.01 (m, 3H), 5.14 (m, 1H), 3.89 (m, 1H), 3.27 (m, |
| | 1H), 2.33 (m, 2H), 2.06-1.89 (m, 1H), 1.81-1.68 (m, 9H), 1.66-1.48 |
| | (m, 3H), 1.49-1.35 (m, 3H), 1.12-0.98 (m, 2H), 0.52 (s, 9H). |
| 225 | ¹H NMR (500 MHz, DMSO-d₆) δ 13.01 (s, 1H), 8.58-8.47 (m, 1H), 7.96- |
| | 7.83 (m, 2H), 7.74-7.53 (m, 2H), 7.37-7.23 (m, 1H), 7.21-7.09 (m, |
| | 2H), 5.21-5.06 (m, 1H), 3.95-3.79 (m, 1H), 3.30-3.17 (m, 1H), 2.11- |
| | 1.90 (m, 4H), 1.87-1.73 (m, 1H), 1.57 (m, 4H), 1.44-1.23 (m, 8H), 0.80 |
| | (m, 2H), 0.55 (s, 9H). |
| 226 | ¹H NMR (500 MHz, DMSO-d₆) δ 8.52 (s, 1H), 7.90 (m, 2H), 7.73-7.55 |
| | (m, 2H), 7.30 (dd, J = 7.6, 7.6 Hz, 1H), 7.17 (d, J = 7.6 Hz, 1H), 7.10 (d, |
| | J = 7.7 Hz, 1H), 5.14 (dd, J = 10.7, 4.0 Hz, 1H), 3.88 (m, 2H), 3.34-3.16 |
| | (m, 1H), 2.02 (s, 3H), 2.00-1.84 (m, 2H), 1.58 (s, 3H), 1.58-1.47 (m, |
| | 2H), 1.40 (d, J = 14.5 Hz, 1H), 0.59 (d, J = 6.6 Hz, 3H), 0.56 (s, 9H), 0.53 |
| | (d, J = 6.6 Hz, 3H). |
| 227 | ¹H NMR (500 MHz, DMSO-d₆) δ 8.54 (s, 1H), 7.91 (m, 2H), 7.67 (m, 2H), |
| | 7.29 (dd, J = 7.6, 7.6 Hz, 1H), 7.13 (dd, J = 20.0, 7.6 Hz, 2H), 5.15 (dd, |
| | J = 10.7, 4.1 Hz, 1H), 3.97-3.85 (m, 1H), 3.30 (q, J = 10.1, 10.1, 8.0 Hz, |
| | 1H), 2.20 (dd, J = 7.4, 4.2 Hz, 2H), 1.73 (m, 4H), 1.60 (s, 3H), 1.54 (dd, |
| | J = 14.6, 8.8 Hz, 1H), 1.41 (d, J = 14.5 Hz, 1H), 0.78 (d, J = 6.6 Hz, 3H), |
| | 0.76 (d, J = 6.5 Hz, 3H), 0.51 (s, 9H). |
| 228 | ¹H NMR (500 MHz, DMSO-d₆) δ 8.57 (s, 1H), 8.28 (d, J = 2.9 Hz, 1H), |
| | 7.94 (d, J = 7.1 Hz, 1H), 7.67 (s, 2H), 7.56 (m, 1H), 7.49 (m, 1H), 7.27 (dd, |
| | J = 7.7, 7.7 Hz, 1H), 7.08 (d, J = 7.7 Hz, 1H), 6.84 (d, J = 7.9 Hz, 1H), |
| | 5.26 (dd, J = 10.8, 4.3 Hz, 1H), 4.84 (d, J = 15.4 Hz, 1H), 4.52 (d, J = 15.4 |
| | Hz, 1H), 4.32 (dd, J = 11.1, 11.1 Hz, 1H), 4.03 (m, 1H), 3.74 (dd, J = 6.1, |
| | 3.7 Hz, 4H), 3.21 (d, J = 4.9, 4.9 Hz, 4H), 1.85 (dd, J = 15.3, 8.8 Hz, 1H), |
| | 1.77 (s, 3H), 1.64 (s, 3H), 1.55-1.43 (m, 1H), 1.37 (d, J = 15.1 Hz, 1H), |
| | 0.80 (d, J = 7.8 Hz, 2H), 0.74-0.66 (m, 1H), 0.51 (m, 10H). |
| 231 | ¹H NMR (400 MHz, DMSO-d₆) δ 13.02 (br. s, 1H), 8.68 (br. s., 1H), 8.35 |
| | (d, J = 6.8 Hz, 1H), 7.96 (br. s., 1H), 7.79-7.61 (m, 2H), 7.38-7.27 (m, |
| | 1H), 7.17 (d, J = 7.6 Hz, 1H), 7.14 (d, J = 7.3 Hz, 1H), 6.90 (br. s., 1H), |
| | 5.54-5.20 (m, 2H), 4.79-4.69 (m, 2H), 4.42 (t, J = 10.8 Hz, 1H), 4.08- |
| | 3.98 (m, 1H), 3.06 (br. s., 3H), 2.27-2.16 (m, 2H), 1.84-1.67 (m, 5H), |
| | 1.60 (br. s., 3H), 1.31-1.10 (m, 8H), 0.79 (t, J = 7.5 Hz, 6H), 0.74 (d, J = |
| | 6.4 Hz, 3H), 0.30-0.21 (m, 3H). |
| 232 | ¹H NMR (400 MHz, DMSO-d₆) δ 13.03 (br. s, 1H), 8.74-8.64 (m, 1H), |
| | 8.38 (d, J = 7.3 Hz, 1H), 7.95 (br. s., 1H), 7.70 (br. s., 2H), 7.32 (t, J = 7.6 |
| | Hz, 1H), 7.21-7.11 (m, 2H), 6.93 (d, J = 7.3 Hz, 1H), 5.55-5.21 (m, 2H), |
| | 4.90-4.67 (m, 3H), 3.88-3.80 (m, 1H), 3.04 (s, 3H), 2.26-2.17 (m, 2H), |
| | 2.16-2.06 (m, 1H), 1.89 (br. s, 3H), 1.82-1.73 (m, 1H), 1.57 (br. s, 3H), |
| | 1.33-1.20 (m, 4H), 1.07 (d, J = 6.6 Hz, 2H), 0.84-0.75 (m, 12H). |
| 233 | ¹H NMR (400 MHz, DMSO-d₆) δ 13.35-12.73 (br. s, 1H), 8.71 (br. s, 1H), |
| | 8.51 (s, 2H), 8.04-7.84 (m, 1H), 7.80-7.50 (m, 2H), 7.41-7.24 (m, 1H), |
| | 7.22-7.03 (m, 2H), 5.42 (m, 1H), 4.88 (d, J = 16.6 Hz, 1H), 4.84-4.77 |
| | (m, 1H), 4.65 (d, J = 16.4 Hz, 1H), 4.23 (m, 1H), 4.04 (m, 1H), 2.28-2.11 |
| | (m, 2H), 1.91-1.68 (m, 5H), 1.65-1.52 (br. s., 3H), 1.41-1.34 (m, 1H), |
| | 1.30 (dd, J = 6.0, 1.6 Hz, 6H), 0.85-0.71 (m, 6H), 0.54 (s, 9H). |
| 234 | ¹H NMR (400 MHz, DMSO-d₆) δ 13.20-12.92 (br. s, 1H), 8.79-8.60 (br. |
| | s, 1H), 8.51 (s, 2H), 8.05-7.84 (br. s, 1H), 7.80-7.53 (br. s, 2H), 7.38- |
| | 7.23 (br. s, 1H), 7.21-7.02 (br. s, 2H), 5.42 (br. s, 1H), 4.89 (d, J = 16.4 |
| | Hz, 1H), 4.85-4.77 (m, 1H), 4.65 (d, J = 16.4 Hz, 1H), 4.31-4.15 (m, |
| | 1H), 4.09-3.97 (m, 1H), 2.29-2.14 (m, 2H), 1.87-1.69 (m, 4H), 1.67- |
| | 1.53 (m, 6H), 1.52-1.33 (m, 4H), 1.32-1.27 (m, 6H), 1.18-1.03 (m, 3H), |
| | 0.88-0.74 (m, 2H), 0.55 (s, 9H). |
| 235 | ¹H NMR (400 MHz, DMSO-d₆) δ 12.96 (br. s, 1H), 8.72 (br. s, 1H), 7.96 |
| | (br. s, 1H), 7.83-7.57 (m, 2H), 7.46 (dd, J = 8.3, 7.3 Hz, 1H), 7.31 (br. s, |
| | 1H), 7.23-7.07 (m, 2H), 6.58 (d, J = 7.3 Hz, 1H), 6.47 (d, J = 8.6 Hz, 1H), |
| | 5.39 (br. s, 1H), 4.97-4.84 (m, 1H), 4.72 (d, J = 16.1 Hz, 1H), 4.39 (d, |
| | J = 15.4 Hz, 1H), 4.32-4.21 (m, 1H), 4.13-4.00 (m, 1H), 2.82 (s, 3H), 2.29- |
| | 2.15 (m, 2H), 1.87-1.68 (m, 5H), 1.66-1.53 (m, 3H), 1.40-1.32 (m, |
| | 1H), 1.16 (d, J = 6.8 Hz, 3H), 1.07 (d, J = 6.8 Hz, 3H), 0.85-0.70 (m, 6H), |
| | 0.52 (br. s, 9H). |
| 219 | ¹H NMR (400 MHz, DMSO-d₆) δ 12.99 (br. s., 1H), 8.67 (br. s., 1H), 8.64 |
| | (s, 2H), 7.90 (br. s., 1H), 7.75-7.56 (m, 2H), 7.31 (br. s., 1H), 7.22-7.07 |
| | (m, 2H), 5.44 (br. s., 1H), 4.92 (d, J = 16.6 Hz, 1H), 4.69 (d, J = 16.9 Hz, |
| | 1H), 4.35-4.18 (m, 1H), 4.06-3.91 (m, 1H), 2.25-2.18 (m, 2H), 1.94- |
| | 1.86 (m, 1H), 1.84-1.67 (m, 6H), 1.57 (br. s., 3H), 1.39 (br. s., 1H), 1.24- |
| | 1.16 (m, 1H), 0.88 (d, J = 6.6 Hz, 6H), 0.81-0.72 (m, 10H), 0.25 (br. s., 3H). |
| 236 | ¹H NMR (400 MHz, DMSO-d₆) δ 12.71 (br. s, 1H), 8.71 (br. s, 1H), 8.29 |
| | (br. s, 2H), 7.91 (d, J = 7.1 Hz, 1H), 7.72-7.55 (m, 2H), 7.36-7.23 (m, |
| | 1H), 7.20-7.06 (m, 2H), 5.48-5.35 (m, 1H), 4.87 (d, J = 16.1 Hz, 1H), |
| | 4.54 (d, J = 16.4 Hz, 1H), 4.18-3.98 (m, 2H), 2.95 (br. s, 6H), 2.27-2.14 |
| | (m, 2H), 1.87-1.68 (m, 5H), 1.58 (br. s, 3H), 1.42-1.33 (m, 1H), 0.79 (d, |
| | J = 6.8 Hz, 3H), 0.76 (d, J = 6.6 Hz, 3H), 0.54 (br. s, 9H). |
| 237 | ¹H NMR (400 MHz, DMSO-d₆) δ 12.91 (br. s, 1H), 8.66 (s, 1H), 7.88 (br. |
| | s., 1H), 7.63 (br. s, 2H), 7.44 (t, J = 7.6 Hz, 1H), 7.33-7.23 (m, 1H), 7.17- |
| | 7.07 (m, 2H), 6.56 (d, J = 7.3 Hz, 1H), 6.30 (d, J = 8.3 Hz, 1H), 5.51- |
| | 5.41 (m, 1H), 4.72 (d, J = 15.9 Hz, 1H), 4.35 (d, J = 15.7 Hz, 1H), 4.29- |
| | 4.16 (m, 2H), 4.08-3.96 (m, 1H), 3.57-3.48 (m, 1H), 3.40-3.34 (m, 1H), |
| | 2.27-2.17 (m, 2H), 2.07-1.91 (m, 3H), 1.83-1.72 (m, 5H), 1.69-1.64 |
| | (m, 1H), 1.57 (s, 3H), 1.37-1.27 (m, 1H), 1.23-1.16 (m, 4H), 0.79 (d, J = |
| | 6.8 Hz, 3H), 0.76 (d, J = 6.6 Hz, 3H), 0.72 (d, J = 6.6 Hz, 3H), 0.23 (d, |
| | J = 6.1 Hz, 3H). |
| 238 | ¹H NMR (400 MHz, DMSO-d₆) δ 12.80 (br. s., 1H), 8.70 (s, 1H), 7.97 (s, |
| | 1H), 7.94 (d, J = 6.8 Hz, 1H), 7.84 (s, 1H), 7.75-7.59 (m, 2H), 7.31 (t, |
| | J = 7.6 Hz, 1H), 7.17 (d, J = 7.8 Hz, 1H), 7.12 (d, J = 7.6 Hz, 1H), 5.41 (dd, |
| | J = 10.4, 4.3 Hz, 1H), 4.89-4.78 (m, 1H), 4.73 (d, J = 16.1 Hz, 1H), 4.48 |
| | (d, J = 16.1 Hz, 1H), 4.34 (t, J = 11.2 Hz, 1H), 4.09-4.00 (m, 1H), 2.90 |
| | (s, 3H), 2.22 (d, J = 7.6 Hz, 2H), 1.84-1.72 (m, 5H), 1.61 (s, 3H), 1.38 (d, |
| | J = 14.9 Hz, 1H), 1.19 (d, J = 6.6 Hz, 3H), 1.10 (d, J = 6.6 Hz, 3H), 0.79 |
| | (t, J = 7.3 Hz, 6H), 0.53 (s, 9H). |
| 239 | ¹H NMR (400 MHz, DMSO-d₆) δ 12.63 (br. s, 1H), 8.80 (br. s, 1H), 8.12 |
| | (d, J = 6.1 Hz, 1H), 7.93 (d, J = 7.1 Hz, 1H), 7.65 (m, 2H), 7.36-7.24 (m, |
| | 1H), 7.16 (d, J = 7.8 Hz, 1H), 7.12 (d, J = 7.3 Hz, 1H), 6.55-6.46 (m, 1H), |
| | 5.51-5.41 (m, 1H), 4.67 (d, J = 16.9 Hz, 1H), 4.44 (d, J = 16.6 Hz, 1H), |
| | 4.30-4.15 (m, 1H), 4.13-4.01 (m, 1H), 3.30-3.27 (m, 1H), 2.87 (br. s, |
| | 3H), 2.26-2.18 (m, 2H), 1.83-1.71 (m, 5H), 1.59 (br. s, 3H), 1.41-1.32 |
| | (m, 1H), 1.19 (d, J = 6.6 Hz, 3H), 1.11-1.04 (m, 3H), 0.80 (d, J = 6.6 Hz, |
| | 3H), 0.77 (d, J = 6.6 Hz, 3H), 0.53 (s, 9H). |
| 240 | ¹H NMR (400 MHz, DMSO-d₆) δ 12.96 (br. s., 1H), 8.66 (br. s., 1H), 8.07 |
| | (s, 2H), 7.95-7.83 (m, 1H), 7.78-7.53 (m, 2H), 7.39-7.25 (m, 1H), 7.20- |
| | 7.09 (m, 2H), 5.45 (br. s., 1H), 4.97 (d, J = 16.1 Hz, 1H), 4.51-4.31 (m, |
| | 3H), 3.76-3.62 (m, 2H), 3.43-3.38 (m, 2H), 3.08 (s, 2H), 2.22-2.17 (m, |
| | 2H), 1.91-1.82 (m, 3H), 1.77 (t, J = 7.0 Hz, 2H), 1.60-1.49 (m, 3H), 1.10 |
| | (s, 6H), 0.83-0.73 (m, 12H). |
| 241 | ¹H NMR (400 MHz, DMSO-d₆) δ 13.01 (br. s., 1H), 8.62 (br. s., 1H), 8.08 |
| | (s, 2H), 7.97-7.82 (m, 1H), 7.78-7.56 (m, 2H), 7.39-7.26 (m, 1H), 7.20- |
| | 7.08 (m, 2H), 5.39 (br. s., 1H), 4.91 (d, J = 16.1 Hz, 1H), 4.49 (d, J = 16.4 |
| | Hz, 1H), 4.13 (t, J = 10.9 Hz, 1H), 4.03-3.91 (m, 1H), 3.39 (t, J = 7.0 Hz, |
| | 2H), 3.08 (s, 2H), 2.24-2.17 (m, 2H), 1.87-1.71 (m, 7H), 1.56 (br. s., 3H), |
| | 1.35 (s, 3H), 1.26-1.21 (m, 1H), 1.20-1.14 (m, 1H), 1.10 (s, 6H), 0.80- |
| | 0.75 (m, 7H), 0.28-0.22 (m, 2H). |
| 242 | ¹H NMR (400 MHz, DMSO-d₆) δ 12.89 (br. s, 1H), 8.61 (br. s., 1H), 8.08 |
| | (s, 2H), 7.87 (br. s., 1H), 7.73-7.54 (m, 2H), 7.39-7.26 (m, 1H), 7.20- |
| | 7.07 (m, 2H), 5.37 (br. s., 1H), 4.95 (d, J = 16.1 Hz, 1H), 4.53 (d, J = 16.4 |
| | Hz, 1H), 4.06-3.98 (m, 2H), 3.39 (t, J = 6.8 Hz, 2H), 3.08 (s, 2H), 2.18 (d, |
| | J = 7.3 Hz, 2H), 1.91 (br. s., 3H), 1.77 (t, J = 7.0 Hz, 2H), 1.74-1.68 (m, |
| | 1H), 1.55 (br. s., 3H), 1.22 (d, J = 5.6 Hz, 3H), 1.10 (s, 6H), 0.77 (t, J = 6.4 |
| | Hz, 6H). |
| 243 | ¹H NMR (400 MHz, DMSO-d₆) δ 13.04 (br. s, 1H), 8.80-8.60 (m, 1H), |
| | 8.07 (s, 2H), 8.00-7.82 (m, 1H), 7.79-7.53 (m, 2H), 7.39-7.25 (m, 1H), |
| | 7.23-7.08 (m, 2H), 5.52-5.32 (m, 1H), 4.87 (d, J = 16.1 Hz, 1H), 4.55 (d, |
| | J = 16.4 Hz, 1H), 4.22-4.06 (m, 1H), 4.04-3.94 (m, 1H), 3.39 (t, J = 7.0 |
| | Hz, 2H), 3.08 (s, 2H), 2.54 (s, 2H), 2.27-2.16 (m, 2H), 1.84-1.69 (m, 6H), |
| | 1.63-1.54 (m, 2H), 1.42-1.31 (m, 1H), 1.10 (s, 6H), 0.86-0.67 (m, 6H), |
| | 0.55 (s, 9H). |
| 244 | ¹H NMR (400 MHz, DMSO-d₆) δ 13.06 (br s, 1H), 8.72 (s, 1H), 8.49 (s, |
| | 2H), 8.01 (br s, 1H), 7.76 (br s, 2H), 7.25 (t, J = 7.6 Hz, 1H), 7.15-7.04 |
| | (m, 2H), 5.56-5.43 (m, 1H), 4.85 (d, J = 16.4 Hz, 1H), 4.67 (d, J = 16.6 |
| | Hz, 1H), 4.33 (br s, 1H), 4.08-3.98 (m, 1H), 3.82-3.70 (m, 4H), 3.27- |
| | 3.17 (m, 4H), 2.30-2.21 (m, 1H), 2.20-2.11 (m, 1H), 1.89-1.80 (m, 1H), |
| | 1.77 (s, 3H), 1.66-1.54 (m, 3H), 1.54-1.35 (m, 4H), 1.14-1.03 (m, 3H), |
| | 0.86-0.74 (m, 2H), 0.57 (s, 9H). |
| 245 | ¹H NMR (400 MHz, DMSO-d₆) δ 13.05 (br s, 1H), 8.71 (s, 1H), 8.49 (s, |
| | 2H), 7.95 (br s, 1H), 7.85-7.65 (m, 2H), 7.24 (t, J = 6.6 Hz, 1H), 7.12 (br |
| | d, J = 7.1 Hz, 1H), 7.03 (br d, J = 7.6 Hz, 1H), 5.61-5.42 (m, 1H), 4.83 |
| | (d, J = 16.4 Hz, 1H), 4.66 (d, J = 16.4 Hz, 1H), 4.31 (br s, 1H), 3.98-3.85 |
| | (m, 1H), 3.81-3.68 (m, 4H), 3.27-3.19 (m, 4H), 2.08-1.94 (m, 4H), 1.92- |
| | 1.81 (m, 2H), 1.59-1.36 (m, 5H), 1.25-1.19 (m, 1H), 1.10-0.88 (m, |
| | 4H), 0.71-0.43 (m, 11H). |
| 246 | ¹H NMR (400 MHz, DMSO-d₆) δ 13.08 (br s, 1H), 8.57 (s, 1H), 8.30-8.23 |
| | (m, 1H), 8.06-7.96 (m, 1H), 7.84-7.72 (m, 2H), 7.41-7.31 (m, 2H), 7.27 |
| | (t, J = 6.6 Hz, 1H), 7.13 (d, J = 7.6 Hz, 1H), 7.08 (d, J = 7.6 Hz, 1H), 5.39- |
| | 5.29 (m, 1H), 4.84 (d, J = 15.2 Hz, 1H), 4.48 (d, J = 15.4 Hz, 1H), 4.42- |
| | 4.30 (m, 1H), 4.05-3.92 (m, 1H), 3.81-3.70 (m, 4H), 3.21-3.11 (m, 4H), |
| | 2.28-2.14 (m, 2H), 1.97-1.86 (m, 1H), 1.84-1.71 (m, 4H), 1.39 (br d, |
| | J = 15.2 Hz, 1H), 0.79 (d, J = 2.0 Hz, 3H), 0.78 (d, J = 2.0 Hz, 3H), 0.54 (s, 9H). |
| 247 | ¹H NMR (400 MHz, DMSO-d₆) δ 13.06 (br s, 1H), 8.56 (s, 1H), 8.31-8.24 |
| | (m, 1H), 8.04-7.94 (m, 1H), 7.81-7.70 (m, 2H), 7.41-7.32 (m, 2H), 7.27 |
| | (t, J = 7.8 Hz, 1H), 7.14 (d, J = 7.3 Hz, 1H), 7.07 (d, J = 7.6 Hz, 1H), 5.33 |
| | (br dd, J = 10.9, 4.0 Hz, 1H), 4.83 (d, J = 15.2 Hz, 1H), 4.48 (d, J = 15.4 |
| | Hz, 1H), 4.34 (t, J = 11.0 Hz, 1H), 4.01-3.88 (m, 1H), 3.82-3.70 (m, 4H), |
| | 3.22-3.11 (m, 4H), 2.04 (s, 3H), 2.01-1.81 (m, 3H), 1.68-1.55 (m, 1H), |
| | 1.39 (br d, J = 14.9 Hz, 1H), 0.63 (d, J = 6.6 Hz, 3H), 0.57 (s, 9H), 0.52 (d, |
| | J = 6.6 Hz, 3H). |
| 248 | ¹H NMR (400 MHz, DMSO-d₆) δ 13.00 (br s, 1H), 8.57 (s, 1H), 8.32-8.24 |
| | (m, 1H), 8.12-7.94 (m, 1H), 7.84-7.70 (m, 2H), 7.42-7.32 (m, 2H), 7.25 |
| | (t, J = 7.1 Hz, 1H), 7.09 (dd, J = 11.6, 7.7 Hz, 2H), 5.33 (br dd, J = 11.1, |
| | 4.0 Hz, 1H), 4.84 (d, J = 15.4 Hz, 1H), 4.48 (d, J = 15.6 Hz, 1H), 4.36 (br |
| | t, J = 11.0 Hz, 1H), 4.05-3.93 (m, 1H), 3.83-3.70 (m, 4H), 3.22-3.11 |
| | (m, 4H), 2.30-2.21 (m, 1H), 2.20-2.10 (m, 1H), 1.98-1.86 (m, 1H), 1.77 |
| | (s, 3H), 1.59 (br s, 3H), 1.55-1.34 (m, 4H), 1.16-1.02 (m, 3H), 0.88-0.76 |
| | (m, 2H), 0.54 (s, 9H). |
| 249 | ¹H NMR (400 MHz, DMSO-d₆) δ 13.09 (br s, 1H), 8.55 (br s, 1H), 8.35- |
| | 8.24 (m, 1H), 8.02-7.88 (m, 1H), 7.75 (br s, 2H), 7.42-7.30 (m, 2H), 7.24 |
| | (br t, J = 7.1 Hz, 1H), 7.12 (br d, J = 7.8 Hz, 1H), 7.02 (br d, J = 7.6 Hz, |
| | 1H), 5.43-5.20 (m, 1H), 4.84 (d, J = 15.4 Hz, 1H), 4.46 (br d, J = 15.2 Hz, |
| | 1H), 4.38-4.18 (m, 1H), 3.89 (br s, 1H), 3.80-3.69 (m, 4H), 3.24-3.11 |
| | (m, 4H), 2.03 (s, 3H), 2.01-1.81 (m, 3H), 1.62-1.35 (m, 5H), 1.26-1.18 |
| | (m, 1H), 1.10-0.91 (m, 4H), 0.73-0.38 (m, 11H). |
| 250 | ¹H NMR (400 MHz, DMSO-d₆) δ 13.01 (br s, 1H), 8.91-8.75 (m, 1H), |
| | 8.67 (d, J = 5.1 Hz, 1H), 8.00-7.85 (m, 1H), 7.76-7.58 (m, 2H), 7.35- |
| | 7.25 (m, 2H), 7.17-7.09 (m, 2H), 5.70-5.42 (m, 1H), 4.87 (d, J = 17.1 |
| | Hz, 1H), 4.72 (d, J = 17.1 Hz, 1H), 4.30-4.17 (m, 1H), 4.07-3.96 (m, |
| | 1H), 3.06-2.97 (m, 1H), 2.32-2.15 (m, 2H), 1.88-1.70 (m, 3H), 1.64- |
| | 1.39 (m, 9H), 1.29-1.22 (m, 7H), 1.16-1.07 (m, 3H), 0.86-0.78 (m, 3H), |
| | 0.56 (s, 9H). |
| 251 | ¹H NMR (400 MHz, DMSO-d₆) δ 13.04 (br s, 1H), 8.73 (s, 3H), 8.03-7.85 |
| | (m, 1H), 7.80-7.52 (m, 2H), 7.31 (br s, 1H), 7.22-7.04 (m, 2H), 5.47 (br |
| | s, 1H), 4.89 (d, J = 16.6 Hz, 1H), 4.72 (br d, J = 16.6 Hz, 1H), 4.37-4.18 |
| | (m, 1H), 4.10-4.00 (m, 1H), 3.99-3.93 (m, 2H), 3.48-3.40 (m, 2H), 2.93- |
| | 2.83 (m, 1H), 2.29-2.13 (m, 2H), 1.86-1.69 (m, 8H), 1.67-1.53 (m, |
| | 6H), 1.53-1.34 (m, 4H), 1.17-1.04 (m, 3H), 0.87-0.75 (m, 2H), 0.55 (s, 9H). |
| 252 | ¹H NMR (400 MHz, DMSO-d₆) δ 13.23-12.93 (m, 1H), 8.69 (d, J = 1.2 |
| | Hz, 1H), 8.64-8.51 (m, 2H), 8.04-7.86 (m, 1H), 7.82-7.54 (m, 2H), 7.37- |
| | 7.24 (m, 1H), 7.22-7.08 (m, 2H), 5.44-5.29 (m, 1H), 4.87 (d, J = 15.6 |
| | Hz, 1H), 4.62 (br d, J = 16.4 Hz, 1H), 4.46-4.33 (m, 1H), 4.10-4.00 (m, |
| | 1H), 4.00-3.92 (m, 2H), 3.52-3.42 (m, 2H), 3.11-3.00 (m, 1H), 2.29- |
| | 2.16 (m, 2H), 1.88-1.69 (m, 8H), 1.61 (br s, 6H), 1.54-1.45 (m, 2H), 1.44- |
| | 1.34 (m, 2H), 1.18-1.06 (m, 3H), 0.88-0.75 (m, 2H), 0.54 (s, 9H). |
| 253 | ¹H NMR (400 MHz, DMSO-d₆) δ 13.04 (br s, 1H), 8.70 (br s, 1H), 8.02 (s, |
| | 2H), 7.93 (br s, 1H), 7.64 (br s, 2H), 7.29 (br s, 1H), 7.19-7.06 (m, 2H), |
| | 5.51-5.34 (m, 1H), 4.87 (d, J = 16.0 Hz, 1H), 4.56 (d, J = 16.6 Hz, 1H), |
| | 4.15 (t, J = 12.0 Hz, 1H), 4.06-3.93 (m, 1H), 3.64 (s, 4H), 2.27-2.13 (m, |
| | 2H), 1.86-1.73 (m, 4H), 1.65-1.54 (m, 6H), 1.48 (d, J = 12.0 Hz, 2H), |
| | 1.37 (d, J = 16.0 Hz, 2H), 1.29 (s, 6H), 1.15-1.06 (m, 3H), 0.87-0.73 (m, |
| | 2H), 0.54 (s, 9H). |
| 254 | ¹H NMR (400 MHz, DMSO-d₆) δ 13.02 (br s, 1H), 8.71 (br s, 1H), 8.07 (s, |
| | 2H), 7.93 (br s, 1H), 7.65 (br s, 2H), 7.31 (br s, 1H), 7.14 (t, J = 8.0 Hz, |
| | 2H), 5.51-5.36 (m, 1H), 4.88 (d, J = 16.1 Hz, 1H), 4.53 (d, J = 16.4 Hz, |
| | 1H), 4.14 (t, J = 12.0 Hz, 1H), 4.05-3.92 (m, 1H), 3.39 (t, J = 7.0 Hz, 2H), |
| | 3.08 (s, 2H), 2.29-2.13 (m, 2H), 1.86-1.71 (m, 6H), 1.66-1.53 (m, 6H), |
| | 1.49 (d, J = 12.0 Hz, 2H), 1.37 (d, J = 16.0 Hz, 2H), 1.15-1.06 (m, 9H), |
| | 0.86-0.73 (m, 2H), 0.55 (s, 9H). |
| 255 | ¹H NMR (400 MHz, DMSO-d₆) δ 13.05 (br s, 1H), 8.70 (br s, 1H), 8.02- |
| | 7.87 (m, 1H), 7.81-7.58 (m, 2H), 7.46 (dd, J = 8.3, 7.3 Hz, 1H), 7.39- |
| | 7.24 (m, 1H), 7.23-7.06 (m, 2H), 6.58 (d, J = 7.3 Hz, 1H), 6.47 (d, J = 8.6 |
| | Hz, 1H), 5.47-5.30 (m, 1H), 4.97-4.85 (m, 1H), 4.73 (d, J = 15.9 Hz, |
| | 1H), 4.39 (d, J = 15.9 Hz, 1H), 4.26 (t, J = 10.3 Hz, 1H), 4.13-4.00 (m, |
| | 1H), 2.82 (s, 3H), 2.29-2.11 (m, 2H), 1.87-1.68 (m, 4H), 1.66-1.31 (m, |
| | 10H), 1.18-1.04 (m, 9H), 0.92-0.74 (m, 2H), 0.52 (s, 9H). |
| 256 | ¹H NMR (400 MHz, DMSO-d₆) δ 13.02 (br s, 1H), 8.67 (br s, 1H), 8.07- |
| | 7.86 (m, 1H), 7.82-7.53 (m, 2H), 7.40 (dd, J = 8.6, 7.3 Hz, 1H), 7.36- |
| | 7.22 (m, 1H), 7.21-7.06 (m, 2H), 6.54 (d, J = 7.3 Hz, 1H), 6.49 (d, J = 8.6 |
| | Hz, 1H), 5.38-5.25 (m, 1H), 4.69 (d, J = 15.9 Hz, 1H), 4.43-4.33 (m, |
| | 3H), 4.30-4.21 (m, 1H), 4.12-4.03 (m, 1H), 2.29-2.16 (m, 2H), 1.87- |
| | 1.71 (m, 4H), 1.68-1.38 (m, 9H), 1.32-1.23 (m, 13H), 1.23-1.05 (m, |
| | 3H), 0.89-0.75 (m, 2H), 0.51 (s, 9H). |
| 257 | ¹H NMR (400 MHz, DMSO-d₆) δ 13.47-12.53 (m, 1H), 8.63 (br s, 1H), |
| | 8.49 (d, J = 1.7 Hz, 1H), 7.94 (br s, 1H), 7.83-7.60 (m, 3H), 7.50-7.39 |
| | (m, 1H), 7.31-7.26 (m, 1H), 7.21-7.08 (m, 2H), 5.37 (br dd, J = 5.7, 4.8 |
| | Hz, 1H), 4.86 (d, J = 15.6 Hz, 1H), 4.55 (br d, J = 15.7 Hz, 1H), 4.30 (t, |
| | J = 11.2 Hz, 1H), 4.07-4.00 (m, 1H), 4.00-3.91 (m, 2H), 3.49-3.40 (m, |
| | 2H), 2.93-2.79 (m, 1H), 2.29-2.13 (m, 2H), 1.93-1.67 (m, 8H), 1.60 (br |
| | s, 6H), 1.53-1.45 (m, 2H), 1.43-1.34 (m, 2H), 1.19-1.05 (m, 3H), 0.89- |
| | 0.74 (m, 2H), 0.53 (s, 9H). |
| 258 | ¹H NMR (400 MHz, DMSO-d₆) δ 13.01 (br s, 1H), 8.90-8.76 (m, 1H), |
| | 8.63 (d, J = 5.1 Hz, 1H), 8.01-7.85 (m, 1H), 7.79-7.54 (m, 2H), 7.38- |
| | 7.23 (m, 2H), 7.21-7.07 (m, 2H), 5.68-5.45 (m, 1H), 4.89 (d, J = 17.1 |
| | Hz, 1H), 4.69 (d, J = 16.9 Hz, 1H), 4.27-4.12 (m, 1H), 4.04-3.90 (m, |
| | 1H), 2.29-2.17 (m, 2H), 1.86-1.69 (m, 4H), 1.68-1.33 (m, 11H), 1.28- |
| | 1.03 (m, 4H), 0.91-0.75 (m, 3H), 0.57 (s, 9H). |
| 259 | ¹H NMR (400 MHz, DMSO-d₆) δ 13.02 (br s, 1H), 8.68 (br s, 1H), 8.01- |
| | 7.86 (m, 1H), 7.83-7.58 (m, 2H), 7.45 (dd, J = 8.6, 7.3 Hz, 1H), 7.39- |
| | 7.23 (m, 1H), 7.21-7.05 (m, 2H), 6.60 (d, J = 7.1 Hz, 1H), 6.55 (d, J = 8.6 |
| | Hz, 1H), 5.45-5.27 (m, 1H), 4.79 (d, J = 15.7 Hz, 1H), 4.34 (d, J = 15.6 |
| | Hz, 1H), 4.27-4.17 (m, 1H), 4.07-3.99 (m, 1H), 3.45 (s, 2H), 3.11 (s, 3H), |
| | 2.27-2.11 (m, 1H), 1.88-1.32 (m, 15H), 1.20-1.03 (m, 3H), 0.90 (s, 9H), |
| | 0.85-0.74 (m, 2H), 0.53 (s, 9H). |
| 260 | ¹H NMR (400 MHz, DMSO-d₆) δ 13.28-12.86 (m, 1H), 8.82-8.62 (m, |
| | 1H), 8.47 (s, 2H), 8.02-7.82 (m, 1H), 7.81-7.54 (m, 2H), 7.40-7.23 (m, |
| | 1H), 7.22-7.02 (m, 2H), 5.54-5.34 (m, 1H), 4.86 (d, J = 16.4 Hz, 1H), |
| | 4.58 (d, J = 16.6 Hz, 1H), 4.26-4.12 (m, 1H), 4.07-3.95 (m, 1H), 3.28- |
| | 3.23 (m, 4H), 2.30-2.16 (m, 2H), 1.87-1.71 (m, 4H), 1.59 (br s, 6H), 1.52- |
| | 1.34 (m, 8H), 1.18-1.05 (m, 3H), 1.00-0.93 (m, 6H), 0.88-0.75 (m, |
| | 2H), 0.63-0.49 (m, 9H). |
| 261 | ¹H NMR (400 MHz, DMSO-d₆) δ 8.82 (s, 1H), 8.14 (d, J = 6.1 Hz, 1H), |
| | 7.95-7.84 (m, 1H), 7.72-7.51 (m, 2H), 7.32-7.22 (m, 1H), 7.16-7.04 |
| | (m, 2H), 6.52 (d, J = 6.4 Hz, 1H), 5.53-5.45 (m, 1H), 4.72 (d, J = 16.6 |
| | Hz, 1H), 4.47-4.37 (m, 1H), 4.19-3.96 (m, 2H), 3.10 (br s, 6H), 2.30- |
| | 2.13 (m, 2H), 1.80-1.68 (m, 4H), 1.64-1.54 (m, 6H), 1.51-1.35 (m, 4H), |
| | 1.15-1.06 (m, 3H), 0.89-0.72 (m, 2H), 0.54 (s, 9H). |
| 262 | ¹H NMR (400 MHz, DMSO-d₆) δ 8.80 (s, 1H), 8.12 (d, J = 6.1 Hz, 1H), |
| | 7.95-7.86 (m, 1H), 7.69-7.57 (m, 2H), 7.31-7.20 (m, 1H), 7.17-7.04 |
| | (m, 2H), 6.58-6.44 (m, 1H), 5.45 (br d, J = 6.6 Hz, 1H), 4.67 (d, J = 16.9 |
| | Hz, 1H), 4.42 (br d, J = 16.9 Hz, 1H), 4.18-4.03 (m, 2H), 2.86 (br s, 3H), |
| | 2.29-2.15 (m, 2H), 1.78-1.69 (m, 4H), 1.63-1.54 (m, 6H), 1.51-1.32 |
| | (m, 4H), 1.20-1.16 (m, 5H), 1.11-1.06 (m, 4H), 0.87-0.73 (m, 2H), 0.53 |
| | (s, 9H). |
| 263 | ¹H NMR (400 MHz, DMSO-d₆) δ 13.54-12.10 (m, 1H), 8.73 (s, 1H), 8.22 |
| | (d, J = 6.1 Hz, 1H), 7.94 (d, J = 6.8 Hz, 1H), 7.76-7.60 (m, 2H), 7.30 (t, |
| | J = 6.8 Hz, 1H), 7.16 (br d, J = 7.8 Hz, 1H), 7.11 (br d, J = 7.6 Hz, 1H), |
| | 6.71 (d, J = 6.4 Hz, 1H), 5.53-5.39 (m, 1H), 4.72 (d, J = 16.9 Hz, 1H), |
| | 4.48 (br d, J = 17.1 Hz, 1H), 4.21 (t, J = 11.0 Hz, 1H), 4.09-3.96 (m, 1H), |
| | 3.77-3.58 (m, 8H), 2.30-2.15 (m, 2H), 1.85-1.70 (m, 4H), 1.69-1.30 |
| | (m, 10H), 1.18-1.06 (m, 3H), 0.89-0.74 (m, 2H), 0.54 (s, 9H). |
| 265 | ¹H NMR (400 MHz, DMSO-d₆) δ 13.74 (s, 1H), 8.73 (s, 1H), 8.37 (d, J = |
| | 7.4 Hz, 1H), 7.99 (s, 1H), 7.71 (s, 2H), 7.32 (t, J = 7.6 Hz, 1H), 7.22 (d, |
| | J = 7.7 Hz, 1H), 7.13 (d, J = 7.5 Hz, 1H), 6.92 (s, 1H), 5.72-5.09 (m, 2H), |
| | 4.93-4.62 (m, 2H), 4.44 (t, J = 11.0 Hz, 1H), 4.09 (s, 1H), 3.05 (s, 3H), |
| | 2.39-2.28 (m, 2H), 2.02 (hept, J = 7.8 Hz, 1H), 1.75 (s, 3H), 1.68 (dd, J = |
| | 15.3, 9.2 Hz, 2H), 1.62 (s, 3H), 1.58-1.50 (m, 3H), 1.49-1.36 (m, 3H), |
| | 1.29 (s, 4H), 1.15-1.01 (m, 4H), 0.54 (s, 9H). |
| 266 | ¹H NMR (400 MHz, DMSO-d₆) δ 13.05 (s, 1H), 8.71 (s, 1H), 8.51 (s, 2H), |
| | 7.94 (s, 1H), 7.66 (s, 2H), 7.31 (s, 1H), 7.22 (s, 1H), 7.13 (s, 1H), 5.42 (s, |
| | 1H), 4.89 (d, J = 16.4 Hz, 1H), 4.80 (hept, J = 6.0 Hz, 1H), 4.65 (d, J = |
| | 16.5 Hz, 1H), 4.32-4.14 (m, 1H), 4.11-3.96 (m, 1H), 2.42-2.23 (m, 2H), |
| | 2.04-1.93 (m, 1H), 1.88-1.80 (m, 1H), 1.78 (s, 3H), 1.59 (s, 7H), 1.46 (s, |
| | 2H), 1.37 (d, J = 15.1 Hz, 1H), 1.34-1.25 (m, 6H), 1.14-0.98 (m, 2H), |
| | 0.54 (s, 9H). |
| 267 | ¹H NMR (400 MHz, DMSO-d₆) δ 12.97 (s, 1H), 8.56 (s, 1H), 8.23 (t, J = |
| | 1.8 Hz, 1H), 7.94 (d, J = 6.8 Hz, 1H), 7.68 (t, J = 6.5 Hz, 2H), 7.42 (d, J = |
| | 1.8 Hz, 2H), 7.31 (t, J = 7.6 Hz, 1H), 7.21 (d, J = 7.7 Hz, 1H), 7.12 (d, J = |
| | 7.5 Hz, 1H), 5.26 (dd, J = 10.9, 4.4 Hz, 1H), 4.85 (d, J = 15.4 Hz, 1H), |
| | 4.70 (hept, J = 6.0 Hz, 1H), 4.49 (d, J = 15.4 Hz, 1H), 4.28 (t, J = 11.2 Hz, |
| | 1H), 4.05-3.92 (m, 1H), 2.40-2.27 (m, 2H), 1.99 (dq, J = 15.1, 7.6 Hz, |
| | 1H), 1.87 (dd, J = 15.2, 8.8 Hz, 1H), 1.76 (s, 3H), 1.60 (s, 3H), 1.58-1.49 |
| | (m, 4H), 1.49-1.41 (m, 2H), 1.37 (d, J = 15.0 Hz, 1H), 1.29 (d, J = 6.0 |
| | Hz, 6H), 1.15-0.98 (m, 2H), 0.52 (s, 9H). |
| 268 | ¹H NMR (400 MHz, DMSO-d₆) δ 13.36-11.72 (broad m, 1H), 8.74 (br s, |
| | 1H), 8.63 (s, 2H), 7.93 (br s, 1H), 7.65 (br s, 2H), 7.39-7.03 (m, 3H), 5.47 |
| | (s, 1H), 4.90 (d, J = 16.8 Hz, 1H), 4.72 (d, J = 16.8 Hz, 1H), 4.25 (s, 1H), |
| | 4.02 (s, 1H), 2.49-2.45 (overlapped with DMSO, m, 2H), 2.35 (s, 2H), |
| | 2.09-1.67 (m, 6H), 1.65-1.30 (m, 10H), 1.14-0.99 (m, 2H), 0.88 (d, J = |
| | 6.6 Hz, 6H), 0.55 (s, 9H). |
| 270 | ¹H NMR (400 MHz, DMSO-d₆) δ 13.27-11.55 (broad m, 1H), 8.70 (s, |
| | 1H), 8.07 (s, 2H), 7.92 (br s, 1H), 7.65 (s, 2H), 7.30 (br s, 1H), 7.21 (br s, |
| | 1H), 7.12 (br s, 1H), 5.41 (br s, 1H), 4.87 (d, J = 16.2 Hz, 1H), 4.53 (d, J = |
| | 16.3 Hz, 1H), 4.21-4.07 (m, 1H), 4.05-3.91 (m, 1H), 3.39 (t, J = 7.0 Hz, |
| | 2H), 3.08 (s, 2H), 2.40-2.26 (m, 2H), 2.04-1.89 (m, 1H), 1.87-1.68 (m, |
| | 6H), 1.66-1.49 (m, 7H), 1.48-1.41 (m, 2H), 1.37 (d, J = 14.9 Hz, 1H), |
| | 1.10 (s, 6H), 1.08-0.99 (m, 2H), 0.55 (s, 9H). |
| 271 | ¹H NMR (400 MHz, DMSO-d₆) δ 13.29-11.62 (broad m, 1H), 8.71 (s, |
| | 1H), 8.07 (s, 2H), 7.93 (br s, 1H), 7.66 (br s, 2H), 7.30 (br s, 1H), 7.21- |
| | 7.05 (m, 2H), 5.42 (br s, 1H), 4.88 (d, J = 16.2 Hz, 1H), 4.53 (d, J = 16.3 |
| | Hz, 1H), 4.13 (t, J = 10.9 Hz, 1H), 4.05-3.95 (m, 1H), 3.83-3.72 (m, 2H), |
| | 3.39 (t, J = 7.0 Hz, 2H), 3.18 (t, J = 11.9 Hz, 2H), 3.08 (s, 2H), 2.36-2.14 |
| | (m, 2H), 1.86-1.69 (m, 6H), 1.68-1.55 (m, 4H), 1.43-1.32 (m, 3H), 1.17- |
| | 1.01 (m, 8H), 0.55 (s, 9H). |
| 272 | ¹H NMR (400 MHz, DMSO-d₆) δ 13.19-11.54 (broad m, 1H), 8.69 (br s, |
| | 1H), 8.07 (s, 2H), 7.89 (br s, 1H), 7.65 (br s, 2H), 7.30 (br s, 1H), 7.19 (br |
| | s, 1H), 7.11 (br s, 1H), 5.42 (s, 1H), 4.86 (d, J = 16.1 Hz, 1H), 4.54 (d, J = |
| | 16.2 Hz, 1H), 4.10 (s, 1H), 3.92 (s, 1H), 3.68 (t, J = 13.8 Hz, 2H), 3.39 (t, |
| | J = 7.0 Hz, 2H), 3.16-2.98 (m, 4H), 2.04 (s, 4H), 1.92-1.82 (m, 1H), 1.77 |
| | (t, J = 7.0 Hz, 2H), 1.59 (s, 3H), 1.52-1.42 (m, 1H), 1.36 (d, J = 14.9 Hz, |
| | 1H), 1.31-1.20 (m, 2H), 1.10 (s, 6H), 0.96-0.77 (m, 3H), 0.59 (s, 9H). |
| 273 | ¹H NMR (400 MHz, DMSO-d₆) δ 13.36-11.43 (broad m, 1H), 8.71 (s, |
| | 1H), 7.95 (br s, 1H), 7.69 (br s, 2H), 7.51 (br s, 1H), 7.30 (t, J = 7.7 Hz, |
| | 1H), 7.21 (d, J = 7.7 Hz, 1H), 7.12 (d, J = 7.5 Hz, 1H), 6.77-6.41 (br m, |
| | 2H), 5.46-5.30 (m, 1H), 4.86 (s, 1H), 4.75 (d, J = 15.7 Hz, 1H), 4.44 (s, |
| | 1H), 4.28 (t, J = 11.0 Hz, 1H), 4.11-3.98 (m, 1H), 2.85 (s, 3H), 2.38-2.27 |
| | (m, 2H), 2.00 (p, J = 7.5 Hz, 1H), 1.87-1.68 (m, 4H), 1.66-1.49 (m, 7H), |
| | 1.48-1.40 (m, 2H), 1.36 (d, J = 15.1 Hz, 1H), 1.18 (d, J = 6.6 Hz, 3H), |
| | 1.14-0.97 (m, 5H), 0.51 (s, 9H). |
| 274 | ¹H NMR (400 MHz, DMSO-d₆) δ 13.69-11.64 (m, 1H), 8.57 (s, 1H), 8.31 |
| | (d, J = 2.9 Hz, 1H), 7.92 (s, 1H), 7.69 (s, 3H), 7.60 (s, 1H), 7.31 (t, J = 7.7 |
| | Hz, 1H), 7.19 (d, J = 7.5 Hz, 1H), 7.12 (d, J = 7.6 Hz, 1H), 5.31 (d, J = 8.8 |
| | Hz, 1H), 4.86 (d, J = 15.4 Hz, 1H), 4.61 (d, J = 15.5 Hz, 1H), 4.32 (t, J = |
| | 11.1 Hz, 1H), 4.01-3.89 (m, 1H), 3.76 (dd, J = 6.1, 3.7 Hz, 4H), 3.72- |
| | 3.64 (overlapped with water, m, 2H), 3.26 (t, J = 4.9 Hz, 4H), 3.08 (dtd, |
| | J = 14.3, 11.6, 2.2 Hz, 2H), 2.12-1.97 (m, 4H), 1.95-1.81 (m, 2H), 1.61 (s, |
| | 3H), 1.48 (s, 1H), 1.38 (d, J = 15.1 Hz, 1H), 1.27 (d, J = 13.1 Hz, 1H), 1.11- |
| | 0.68 (m, 3H), 0.56 (s, 9H). |
| 275 | ¹H NMR (400 MHz, DMSO-d₆) δ 13.62-11.09 (m, 1H), 8.61 (s, 1H), 8.31 |
| | (d, J = 2.9 Hz, 1H), 7.97 (d, J = 6.2 Hz, 1H), 7.81-7.56 (m, 4H), 7.31 (t, |
| | J = 7.6 Hz, 1H), 7.19 (d, J = 7.7 Hz, 1H), 7.14 (d, J = 7.5 Hz, 1H), 5.33 |
| | (dd, J = 10.7, 4.3 Hz, 1H), 4.88 (d, J = 15.5 Hz, 1H), 4.64 (d, J = 15.5 Hz, |
| | 1H), 4.37 (t, J = 11.1 Hz, 1H), 4.08-3.98 (m, 1H), 3.81-3.74 (overlapped |
| | with water, m, 6H), 3.28 (t, J = 4.9 Hz, 4H), 3.18 (tt, J = 11.6, 2.5 Hz, 2H), |
| | 2.35-2.17 (m, 2H), 1.83 (dd, J = 15.4, 8.9 Hz, 1H), 1.77 (s, 3H), 1.69- |
| | 1.57 (m, 4H), 1.43-1.29 (m, 3H), 1.17-1.00 (m, 2H), 0.52 (s, 9H). |
| 276 | ¹H NMR (400 MHz, DMSO-d₆) δ 13.23-11.60 (broad m, 1H), 8.74 (s, |
| | 1H), 8.69 (s, 2H), 7.93 (s, 1H), 7.65 (br s, 2H), 7.30 (s, 1H), 7.13 (s, 2H), |
| | 5.47 (br s, 1H), 4.89 (d, J = 16.7 Hz, 1H), 4.70 (d, J = 16.8 Hz, 1H), 4.24 |
| | (br s, 1H), 4.10-3.95 (m, 1H), 2.64-2.55 (m, 1H), 2.31-2.08 (m, 2H), |
| | 1.86-1.75 (m, 7H), 1.74-1.67 (m, 2H), 1.65-1.55 (m, 6H), 1.53-1.29 |
| | (m, 9H), 1.15-1.04 (m, 3H), 0.85-0.74 (m, 2H), 0.55 (s, 9H). |
| 277 | ¹H NMR (400 MHz, DMSO-d₆) δ 13.27-10.65 (broad m, 1H), 8.63 (s, |
| | 1H), 8.43 (d, J = 2.3 Hz, 1H), 7.99-7.88 (m, 1H), 7.76-7.60 (m, 3H), 7.38 |
| | (d, J = 8.1 Hz, 1H), 7.34-7.25 (m, 1H), 7.13 (dd, J = 12.5, 7.8 Hz, 2H), |
| | 5.34 (d, J = 8.9 Hz, 1H), 4.86 (d, J = 15.7 Hz, 1H), 4.52 (d, J = 15.7 Hz, |
| | 1H), 4.27 (t, J = 11.2 Hz, 1H), 4.07-3.95 (m, 1H), 2.60-2.56 (overlapped |
| | with DMSO, m, 1H), 2.30-2.11 (m, 2H), 1.91-1.68 (m, 9H), 1.66-1.53 |
| | (m, 6H), 1.53-1.29 (m, 9H), 1.15-1.04 (m, 3H), 0.88-0.72 (m, 2H), 0.53 |
| | (s, 9H). |
| 278 | ¹H NMR (400 MHz, DMSO-d₆) δ 13.23-11.41 (broad m, 1H), 8.48 (s, |
| | 1H), 8.16 (d, J = 1.4 Hz, 1H), 8.13 (d, J = 1.5 Hz, 1H), 7.92 (br s, 1H), 7.66 |
| | (s, 2H), 7.37-7.21 (m, 1H), 7.20-7.01 (m, 2H), 5.32-5.13 (m, 1H), 4.76 |
| | (d, J = 14.9 Hz, 1H), 4.49-4.33 (m, 2H), 4.03-3.91 (m, 1H), 3.71 (td, J = |
| | 5.6, 2.2 Hz, 2H), 3.52 (t, J = 5.6 Hz, 2H), 3.25 (s, 3H), 3.08 (s, 3H), 2.29- |
| | 2.13 (m, 2H), 1.96-1.85 (m, 1H), 1.77 (s, 3H), 1.67-1.54 (m, 6H), 1.53- |
| | 1.45 (m, 2H), 1.43-1.34 (m, 2H), 1.18-1.01 (m, 3H), 0.86-0.74 (m, 2H), |
| | 0.52 (s, 9H). |
| 279 | ¹H NMR (400 MHz, DMSO-d₆) δ 13.39-11.39 (broad m, 1H), 8.57 (s, |
| | 1H), 8.27-8.19 (m, 1H), 8.02-7.86 (m, 1H), 7.68 (br s, 2H), 7.46-7.37 |
| | (m, 2H), 7.30 (t, J = 7.8 Hz, 1H), 7.14 (dd, J = 12.7, 7.7 Hz, 2H), 5.26 (d, |
| | J = 9.2 Hz, 1H), 4.86 (dd, J = 15.4, 2.3 Hz, 1H), 4.69 (pd, J = 6.1, 2.3 Hz, |
| | 1H), 4.48 (dd, J = 15.4, 2.3 Hz, 1H), 4.27 (t, J = 11.3 Hz, 1H), 4.08-3.93 |
| | (m, 1H), 2.29-2.10 (m, 2H), 1.93-1.82 (m, 1H), 1.77 (s, 3H), 1.60 (s, 6H), |
| | 1.53-1.46 (m, 2H), 1.44-1.35 (m, 2H), 1.29 (dd, J = 6.1, 2.2 Hz, 6H), |
| | 1.16-1.06 (m, 3H), 0.84-0.75 (m, 2H), 0.52 (s, 9H). |
| 280 | ¹H NMR (400 MHz, DMSO-d₆) δ 13.23-10.96 (broad m, 1H), 8.70 (s, |
| | 1H), 8.50 (s, 2H), 7.91 (s, 1H), 7.66 (s, 2H), 7.38-7.24 (m, 1H), 7.17 (t, |
| | J = 8.0 Hz, 2H), 5.52-5.31 (m, 1H), 4.86 (d, J = 16.4 Hz, 1H), 4.61 (d, J = |
| | 16.5 Hz, 1H), 4.16 (t, J = 11.1 Hz, 1H), 4.06-3.89 (m, 1H), 3.83-3.70 (m, |
| | 4H), 3.26-3.21 (m, 4H), 2.12-1.99 (m, 5H), 1.93-1.76 (m, 2H), 1.59 (s, |
| | 3H), 1.52-1.21 (m, 7H), 0.93-0.72 (m, 2H), 0.59 (s, 9H). |
| 281 | ¹H NMR (400 MHz, DMSO-d₆) δ 13.29-11.25 (broad m, 1H), 8.71 (s, |
| | 1H), 8.49 (s, 2H), 7.94 (s, 1H), 7.67 (s, 2H), 7.38-7.25 (m, 1H), 7.20 (d, |
| | J = 7.8 Hz, 1H), 7.12 (d, J = 7.6 Hz, 1H), 5.52-5.32 (m, 1H), 4.88 (d, J = |
| | 16.4 Hz, 1H), 4.60 (d, J = 16.5 Hz, 1H), 4.18 (t, J = 11.0 Hz, 1H), 4.09- |
| | 3.96 (m, 1H), 3.84-3.71 (m, 4H), 3.27-3.20 (m, 4H), 2.42-2.22 (m, 2H), |
| | 2.08-1.93 (m, 1H), 1.89-1.70 (m, 4H), 1.69-1.41 (m, 9H), 1.37 (d, J = |
| | 15.1 Hz, 1H), 1.16-0.97 (m, 2H), 0.55 (s, 9H). |
| 282 | ¹H NMR (400 MHz, DMSO-d₆) δ 13.50-11.46 (broad m, 1H), 8.70 (s, |
| | 1H), 8.32 (s, 2H), 7.93 (s, 1H), 7.65 (broad s, 2H), 7.30 (s, 1H), 7.13 (s, |
| | 2H), 5.41 (br s, 1H), 4.87 (d, J = 16.2 Hz, 1H), 4.53 (d, J = 16.3 Hz, 1H), |
| | 4.15 (t, J = 11.0 Hz, 1H), 4.06-3.94 (m, 1H), 3.26-3.18 (m, 2H), 3.01 (s, |
| | 3H), 2.29-2.03 (m, 2H), 1.87-1.69 (m, 4H), 1.64-1.33 (m, 10H), 1.18- |
| | 1.03 (m, 3H), 0.95 (s, 9H), 0.84-0.75 (m, 2H), 0.54 (s, 9H). |
| 283 | ¹H NMR (400 MHz, DMSO-d₆) δ 13.27-11.34 (broad m, 1H), 8.71 (s, |
| | 1H), 8.28 (s, 2H), 7.93 (s, 1H), 7.66 (br s, 2H), 7.29 (s, 1H), 7.13 (t, J = 9.3 |
| | Hz, 2H), 5.42 (d, J = 10.3 Hz, 1H), 4.88 (d, J = 16.3 Hz, 1H), 4.54 (d, J = |
| | 16.4 Hz, 1H), 4.14 (t, J = 11.2 Hz, 1H), 4.08-3.95 (m, 1H), 3.61-3.53 (m, |
| | 2H), 3.53-3.48 (m, 2H), 3.25 (s, 3H), 2.97 (s, 3H), 2.28-2.09 (m, 2H), |
| | 1.90-1.71 (m, 4H), 1.65-1.53 (m, 6H), 1.53-1.44 (m, 2H), 1.43-1.31 |
| | (m, 2H), 1.19-1.04 (m, 3H), 0.88-0.75 (m, 2H), 0.55 (s, 9H). |
| 284 | ¹H NMR (400 MHz, DMSO-d₆) δ 13.43-10.56 (broad m, 1H), 8.53 (s, |
| | 1H), 8.32 (d, J = 2.9 Hz, 1H), 7.89 (d, J = 7.3 Hz, 1H), 7.81-7.51 (m, 4H), |
| | 7.29 (t, J = 7.6 Hz, 1H), 7.18 (d, J = 7.6 Hz, 1H), 7.08 (d, J = 7.6 Hz, 1H), |
| | 5.30 (dd, J = 10.6, 4.3 Hz, 1H), 4.89 (d, J = 15.6 Hz, 1H), 4.63 (d, J = 15.6 |
| | Hz, 1H), 4.34 (t, J = 11.1 Hz, 1H), 3.93-3.83 (m, 1H), 3.80-3.74 |
| | (overlapped with water, m, 4H), 3.27 (overlapped with water, t, J = 4.9 Hz, |
| | 4H), 2.05 (s, 3H), 2.02-1.88 (m, 2H), 1.88-1.78 (m, 1H), 1.60 (s, 3H), |
| | 1.54-1.47 (m, 2H), 1.44-1.33 (m, 2H), 1.31-1.21 (m, 2H), 1.20-1.06 |
| | (m, 3H), 0.97-0.92 (m, 2H), 0.72 (d, J = 6.5 Hz, 3H), 0.67-0.45 (m, 2H), |
| | 0.26 (d, J = 6.3 Hz, 3H). |
| 285 | ¹H NMR (400 MHz, DMSO-d₆) δ 13.62-11.12 (m, 1H), 8.54 (s, 1H), 8.30 |
| | (d, J = 2.9 Hz, 1H), 7.93 (d, J = 6.7 Hz, 1H), 7.80-7.46 (m, 4H), 7.30 (t, |
| | J = 7.7 Hz, 1H), 7.13 (t, J = 8.1 Hz, 2H), 5.28 (dd, J = 10.7, 4.3 Hz, 1H), |
| | 4.89 (d, J = 15.5 Hz, 1H), 4.57 (d, J = 15.6 Hz, 1H), 4.33 (t, J = 11.1 Hz, |
| | 1H), 4.03-3.90 (m, 1H), 3.76 (dd, J = 6.0, 3.7 Hz, 4H), 3.25 (overlapped |
| | with water, t, J = 4.9 Hz, 4H), 2.28-2.09 (m, 2H), 1.87-1.73 (m, 4H), |
| | 1.67-1.55 (m, 6H), 1.52-1.43 (m, 2H), 1.43-1.33 (m, 1H), 1.30-1.19 |
| | (m, 2H), 1.15-1.04 (m, 3H), 0.88-0.76 (m, 2H), 0.69 (d, J = 6.3 Hz, 3H), |
| | 0.22 (d, J = 6.0 Hz, 3H). |
| 286 | ¹H NMR (400 MHz, DMSO-d₆) δ 13.77-10.73 (broad m, 1H), 8.61 (s, |
| | 1H), 8.31 (d, J = 2.9 Hz, 1H), 7.97 (d, J = 6.3 Hz, 1H), 7.81-7.51 (m, 4H), |
| | 7.33 (t, J = 7.6 Hz, 1H), 7.18 (dd, J = 9.9, 7.6 Hz, 2H), 5.29 (dd, J = 10.8, |
| | 4.4 Hz, 1H), 4.87 (d, J = 15.4 Hz, 1H), 4.61 (d, J = 15.5 Hz, 1H), 4.37 (t, |
| | J = 11.2 Hz, 1H), 4.11-4.00 (m, 1H), 3.76 (dd, J = 6.0, 3.8 Hz, 4H), 3.26 |
| | (t, J = 4.9 Hz, 4H), 2.15-1.95 (m, 5H), 1.84 (dd, J = 15.3, 8.8 Hz, 1H), |
| | 1.63 (s, 3H), 1.37 (d, J = 15.0 Hz, 1H), 0.86 (t, J = 7.5 Hz, 3H), 0.52 (s, 9H) |
| 287 | ¹H NMR (400 MHz, DMSO-d₆) δ 13.78-10.82 (broad m, 1H), 8.61 (s, |
| | 1H), 8.31 (d, J = 2.9 Hz, 1H), 7.97 (d, J = 6.3 Hz, 1H), 7.81-7.51 (m, 4H), |
| | 7.33 (t, J = 7.6 Hz, 1H), 7.22 (d, J = 7.7 Hz, 1H), 7.13 (d, J = 7.5 Hz, 1H), |
| | 5.30 (dd, J = 10.6, 4.4 Hz, 1H), 4.87 (d, J = 15.5 Hz, 1H), 4.62 (d, J = 15.5 |
| | Hz, 1H), 4.37 (t, J = 11.2 Hz, 1H), 4.09-3.98 (m, 1H), 3.80-3.73 |
| | (overlapped with water, m, 4H), 3.27 (t, J = 4.9 Hz, 4H), 2.34 (q, J = 7.2 |
| | Hz, 2H), 1.84 (dd, J = 15.3, 8.9 Hz, 1H), 1.77 (s, 3H), 1.62 (s, 3H), 1.39 (d, |
| | J = 15.0 Hz, 1H), 1.06 (t, J = 7.6 Hz, 3H), 0.52 (s, 9H). |
| 288 | ¹H NMR (400 MHz, DMSO-d₆) δ 13.92-11.02 (broad m, 1H), 8.58 (s, |
| | 1H), 8.31 (d, J = 2.9 Hz, 1H), 7.93 (d, J = 5.7 Hz, 1H), 7.81-7.53 (m, 4H), |
| | 7.31 (t, J = 7.6 Hz, 1H), 7.17 (t, J = 8.2 Hz, 2H), 5.30 (dd, J = 10.7, 4.4 |
| | Hz, 1H), 4.88 (d, J = 15.4 Hz, 1H), 4.63 (d, J = 15.5 Hz, 1H), 4.33 (t, J = |
| | 11.1 Hz, 1H), 4.05-3.93 (m, 1H), 3.76 (overlapped with water, dd, J = 6.0, |
| | 3.7 Hz, 4H), 3.27 (t, J = 4.8 Hz, 4H), 2.15-1.97 (m, 5H), 1.93-1.76 (m, |
| | 2H), 1.61 (s, 3H), 1.51-1.26 (m, 7H), 0.91-0.73 (m, 2H), 0.55 (s, 9H). |
| 289 | ¹H NMR (400 MHz, DMSO-d₆) δ 13.80-10.82 (broad m, 1H), 8.60 (s, |
| | 1H), 8.31 (d, J = 2.9 Hz, 1H), 7.96 (d, J = 6.4 Hz, 1H), 7.81-7.53 (m, 4H), |
| | 7.31 (t, J = 7.6 Hz, 1H), 7.21 (d, J = 7.7 Hz, 1H), 7.13 (d, J = 7.5 Hz, 1H), |
| | 5.31 (dd, J = 10.9, 4.4 Hz, 1H), 4.89 (d, J = 15.4 Hz, 1H), 4.61 (d, J = 15.4 |
| | Hz, 1H), 4.35 (t, J = 11.1 Hz, 1H), 4.07-3.97 (m, 1H), 3.80-3.73 |
| | (overlapped with water , m, 4H), 3.27 (t, J = 4.8 Hz, 4H), 2.41-2.24 (m, |
| | 2H), 1.98 (hept, J = 7.7 Hz, 1H), 1.84 (dd, J = 15.4, 8.9 Hz, 1H), 1.76 (s, |
| | 3H), 1.66-1.42 (m, 9H), 1.38 (d, J = 15.2 Hz, 1H), 1.17-0.95 (m, 2H), |
| | 0.52 (s, 9H). |
| 290 | ¹H NMR (400 MHz, DMSO-d₆) δ 13.43-11.37 (broad m, 1H), 8.59 (br s, |
| | 1H), 8.09 (d, J = 3.0 Hz, 1H), 7.96 (br s, 1H), 7.83-7.41 (m, 4H), 7.30 (t, |
| | J = 7.7 Hz, 1H), 7.14 (t, J = 9.5 Hz, 2H), 5.31 (d, J = 10.1 Hz, 1H), 4.85 |
| | (d, J = 15.4 Hz, 1H), 4.69-4.48 (m, 1H), 4.36 (t, J = 11.0 Hz, 1H), 4.12- |
| | 3.95 (m, 1H), 3.63-3.57 (m, 2H), 3.52 (t, J = 5.2 Hz, 2H), 3.25 (s, 3H), |
| | 3.01 (s, 3H), 2.28-2.10 (m, 2H), 1.88-1.73 (m, 4H), 1.67-1.54 (m, 6H), |
| | 1.53-1.44 (m, 2H), 1.38 (d, J = 15.0 Hz, 2H), 1.19-1.00 (m, 3H), 0.87- |
| | 0.80 (m, 2H), 0.51 (s, 9H). |
| 291 | ¹H NMR (400 MHz, DMSO-d₆) δ 13.57-11.25 (broad m, 1H), 8.61 (s, |
| | 1H), 8.10 (d, J = 2.8 Hz, 1H), 8.03-7.91 (m, 1H), 7.88-7.61 (m, 4H), 7.33 |
| | (t, J = 7.6 Hz, 1H), 7.20 (d, J = 7.6 Hz, 1H), 7.13 (d, J = 7.7 Hz, 1H), 5.34 |
| | (dd, J = 10.7, 4.4 Hz, 1H), 4.85 (d, J = 15.4 Hz, 1H), 4.75 (d, J = 15.7 Hz, |
| | 1H), 4.45 (t, J = 11.1 Hz, 1H), 4.11-3.98 (m, 1H), 3.72-3.62 (overlapped |
| | with water m, 4H), 3.24 (s, 3H), 3.04 (s, 3H), 2.05 (s, 3H), 2.02-1.88 (m, |
| | 2H), 1.88-1.74 (m, 1H), 1.68-1.53 (m, 4H), 1.37 (d, J = 15.3 Hz, 1H), |
| | 0.61 (d, J = 6.5 Hz, 3H), 0.58-0.49 (m, 12H). |
| 292 | ¹H NMR (400 MHz, DMSO-d₆) δ 13.75-11.66 (broad m, 1H), 8.57 (s, |
| | 1H), 8.08 (d, J = 2.9 Hz, 1H), 7.95 (br d, J = 6.4 Hz, 1H), 7.77-7.59 (m, |
| | 2H), 7.58-7.34 (m, 2H), 7.31 (t, J = 7.7 Hz, 1H), 7.17 (d, J = 7.8 Hz, 1H), |
| | 7.13 (d, J = 7.6 Hz, 1H), 5.27 (d, J = 8.1 Hz, 1H), 4.85 (d, J = 15.2 Hz, |
| | 1H), 4.52 (d, J = 14.7 Hz, 1H), 4.33 (t, J = 11.3 Hz, 1H), 4.09-3.92 (m, |
| | 1H), 3.61-3.56 (m, 2H), 3.51 (t, J = 5.3 Hz, 2H), 3.24 (overlapped with |
| | water s, 3H), 2.99 (s, 3H), 2.28-2.14 (m, 2H), 1.85 (dd, J = 15.3, 8.8 Hz, |
| | 1H), 1.80-1.69 (m, 4H), 1.61 (s, 3H), 1.37 (d, J = 15.2 Hz, 1H), 0.83- |
| | 0.73 (m, 6H), 0.51 (s, 9H). |
| 293 | ¹H NMR (400 MHz, DMSO-d₆) δ 13.87-12.35 (broad m, 1H), 8.69 (s, |
| | 1H), 8.29 (s, 2H), 7.92 (d, J = 7.1 Hz, 1H), 7.73-7.58 (m, 2H), 7.32 (t, J |
| | = 7.6 Hz, 1H), 7.19 (d, J = 7.6 Hz, 1H), 7.12 (d, J = 7.7 Hz, 1H), 5.41 (dd, |
| | J = 10.8, 4.2 Hz, 1H), 4.85 (d, J = 16.2 Hz, 1H), 4.56 (d, J = 16.3 Hz, 1H), |
| | 4.15 (t, J = 11.1 Hz, 1H), 4.05-3.96 (overlapped with water m, 1H), 3.57 |
| | (t, J = 5.5 Hz, 2H), 3.51 (t, J = 5.1 Hz, 2H), 3.24 (s, 3H), 2.97 (s, 3H), 2.04 |
| | (s, 3H), 2.01-1.90 (m, 2H), 1.86 (dd, J = 15.5, 9.6 Hz, 1H), 1.64-1.51 (m, |
| | 4H), 1.35 (d, J = 15.1 Hz, 1H), 0.61 (d, J = 6.5 Hz, 3H), 0.58 (s, 9H), 0.56 |
| | (d, J = 6.6 Hz, 3H). |
| 294 | ¹H NMR (400 MHz, DMSO-d₆) δ 13.48-12.66 (broad m, 1H), 8.71 (s, |
| | 1H), 8.29 (s, 2H), 7.94 (d, J = 7.2 Hz, 1H), 7.79-7.57 (m, 2H), 7.31 (t, |
| | J = 7.6 Hz, 1H), 7.16 (d, J = 7.7 Hz, 1H), 7.13 (d, J = 7.5 Hz, 1H), 5.42 (dd, |
| | J = 10.8, 4.3 Hz, 1H), 4.86 (d, J = 16.3 Hz, 1H), 4.55 (d, J = 16.4 Hz, 1H), |
| | 4.16 (t, J = 11.2 Hz, 1H), 4.07-3.96 (m, 1H), 3.56 (overlapped with water, |
| | t, J = 5.1 Hz, 2H), 3.50 (t J = 4.9 Hz, 2H), 3.24 (s, 3H), 2.97 (s, 3H), 2.30- |
| | 2.13 (m, 2H), 1.90-1.67 (m, 5H), 1.59 (s, 3H), 1.37 (d, J = 15.0 Hz, 1H), |
| | 0.83-0.74 (m, 6H), 0.55 (s, 9H). |
| 295 | ¹H NMR (400 MHz, DMSO-d₆) δ 13.41-11.20 (broad m, 1H), 8.70 (s, |
| | 1H), 8.50 (s, 2H), 7.92 (s, 1H), 7.65 (s, 2H), 7.32 (t, J = 7.8 Hz, 1H), 7.19 |
| | (d, J = 7.6 Hz, 1H), 7.12 (d, J = 7.7 Hz, 1H), 5.42 (dd, J = 10.9, 4.3 Hz, |
| | 1H), 4.85 (d, J = 16.4 Hz, 1H), 4.62 (d, J = 16.4 Hz, 1H), 4.19 (t, J = 11.1 |
| | Hz, 1H), 4.07-3.94 (m, 1H), 3.75 (dd, J = 6.1, 3.6 Hz, 4H), 3.23 (dd, J = |
| | 5.9, 3.7 Hz, 4H), 2.04 (s, 3H), 2.01-1.89 (m, 2H), 1.85 (dd, J = 15.2, 9.1 |
| | Hz, 1H), 1.64-1.53 (m, 4H), 1.35 (d, J = 15.1 Hz, 1H), 0.62 (d, J = 6.5 |
| | Hz, 3H), 0.58 (s, 9H), 0.56 (d, J = 6.8 Hz, 3H). |
| 296 | ¹H NMR (400 MHz, DMSO-d₆) δ 13.32-11.42 (broad m, 1H), 8.71 (s, |
| | 1H), 8.54-8.42 (m, 2H), 7.93 (s, 1H), 7.65 (s, 2H), 7.31 (s, 1H), 7.15 (s, |
| | 2H), 5.43 (br s, 1H), 4.87 (dd, J = 16.6, 4.5 Hz, 1H), 4.60 (dd, J = 16.6, 4.6 |
| | Hz, 1H), 4.26-4.10 (br m, 1H), 4.07-3.96 (br m, 1H), 3.75 (q, J = 4.8 Hz, |
| | 4H), 3.23 (t, J = 4.9 Hz, 4H), 2.26-2.15 (m, 2H), 1.89-1.68 (m, 5H), 1.59 |
| | (s, 3H), 1.37 (d, J = 15.4 Hz, 1H), 0.89-0.71 (m, 6H), 0.54 (s, 9H). |
| 297 | ¹H NMR (400 MHz, DMSO-d₆) δ 13.56-12.40 (broad m, 1H), 8.59 (s, |
| | 1H), 8.32 (d, J = 2.9 Hz, 1H), 7.95 (d, J = 6.3 Hz, 1H), 7.87-7.55 (m, 4H), |
| | 7.32 (t, J = 7.7 Hz, 1H), 7.20 (d, J = 7.6 Hz, 1H), 7.12 (d, J = 7.7 Hz, 1H), |
| | 5.32 (dd, J = 10.7, 4.4 Hz, 1H), 4.87 (d, J = 15.4 Hz, 1H), 4.66 (d, J = 15.6 |
| | Hz, 1H), 4.37 (t, J = 11.1 Hz, 1H), 4.09-3.95 (m, 1H), 3.76 (overlapped |
| | with water, t, J = 4.8 Hz, 4H), 3.28 (overlapped with water, t, J = 4.8 Hz, |
| | 4H), 2.04 (s, 3H), 1.99-1.80 (m, 3H), 1.61 (s, 3H), 1.59-1.53 (m, 1H), |
| | 1.36 (d, J = 15.2 Hz, 1H), 0.61 (d, J = 6.5 Hz, 3H), 0.58-0.50 (m, 12H). |
| 298 | ¹H NMR (400 MHz, DMSO-d₆) δ 13.56-11.05 (broad m, 1H), 8.57 (s, |
| | 1H), 8.29 (d, J = 2.8 Hz, 1H), 7.94 (d, J = 6.2 Hz, 1H), 7.68 (s, 2H), 7.58- |
| | 7.38 (m, 2H), 7.31 (t, J = 7.6 Hz, 1H), 7.21-7.06 (m, 2H), 5.27 (dd, J = |
| | 11.1, 4.3 Hz, 1H), 4.86 (d, J = 15.3 Hz, 1H), 4.50 (d, J = 15.4 Hz, 1H), |
| | 4.30 (t, J = 11.2 Hz, 1H), 4.07-3.93 (m, 1H), 3.75 (dd, J = 6.0, 3.7 Hz, |
| | 4H), 3.21 (t, J = 4.8 Hz, 4H), 2.21 (d, J = 7.5 Hz, 2H), 1.86 (dd, J = 15.3, |
| | 8.9 Hz, 1H), 1.77 (s, 3H), 1.74-1.69 (m, 1H), 1.60 (s, 3H), 1.37 (d, J = |
| | 14.7 Hz, 1H), 0.82-0.73 (m, 6H), 0.52 (s, 9H). |
| 299 | ¹H NMR (400 MHz, DMSO-d₆). One H missing, likely under DMSO peak. |
| | 8 13.52-11.26 (m, 1H), 8.59 (s, 1H), 8.30 (d, J = 2.8 Hz, 1H), 7.95 (d, J = |
| | 6.6 Hz, 1H), 7.75-7.45 (m, 4H), 7.36 (t, J = 7.7 Hz, 1H), 7.30 (d, J = 7.8 |
| | Hz, 1H), 7.12 (d, J = 7.4 Hz, 1H), 5.28 (dd, J = 11.2, 4.4 Hz, 1H), 4.85 (d, |
| | J = 15.4 Hz, 1H), 4.56 (d, J = 15.4 Hz, 1H), 4.34 (t, J = 11.2 Hz, 1H), 4.11- |
| | 3.97 (m, 1H), 3.75 (dd, J = 6.0, 3.7 Hz, 4H), 3.24 (t, J = 4.8 Hz, 4H), 1.85 |
| | (dd, J = 15.3, 8.9 Hz, 1H), 1.76 (s, 3H), 1.61 (s, 3H), 1.38 (d, J = 15.3 Hz, |
| | 1H), 1.15 (d, J = 6.7 Hz, 3H), 1.09 (d, J = 6.8 Hz, 3H), 0.52 (s, 9H). |
| 300 | ¹H NMR (400 MHz, DMSO-d₆) δ 14.09-12.19 (broad m, 1H), 8.62 (s, |
| | 1H), 8.32 (d, J = 2.9 Hz, 1H), 7.98 (d, J = 6.2 Hz, 1H), 7.84-7.57 (m, 4H), |
| | 7.37 (t, J = 7.7 Hz, 1H), 7.26 (d, J = 7.8 Hz, 1H), 7.19 (d, J = 7.5 Hz, 1H), |
| | 5.32 (dd, J = 11.2, 4.4 Hz, 1H), 4.86 (d, J = 15.4 Hz, 1H), 4.65 (d, J = 15.5 |
| | Hz, 1H), 4.42 (t, J = 11.2 Hz, 1H), 4.18-4.03 (m, 1H), 3.76 (overlapped |
| | with water, dd, J = 6.0, 3.7 Hz, 4H), 3.28 (overlapped with water, t, J = 4.8 |
| | Hz, 4H), 2.13-2.06 (m, 1H), 2.04 (s, 3H), 1.82 (dd, J = 15.4, 8.8 Hz, 1H), |
| | 1.62 (s, 3H), 1.36 (d, J = 15.2 Hz, 1H), 0.97 (d, J = 6.6 Hz, 3H), 0.95 (d, |
| | J = 6.8 Hz, 3H), 0.50 (s, 9H). |
| 301 | ¹H NMR (400 MHz, DMSO-d₆) δ 13.60-11.11 (broad m, 1H), 8.54 (s, |
| | 1H), 8.29 (d, J = 2.5 Hz, 1H), 7.89 (br s, 1H), 7.66 (br s, 2H), 7.51-7.41 |
| | (m, 2H), 7.30 (t, J = 7.6 Hz, 1H), 7.17 (d, J = 7.6 Hz, 1H), 7.09 (d, J = 7.7 |
| | Hz, 1H), 5.27 (dd, J = 10.8, 4.3 Hz, 1H), 4.87 (d, J = 15.3 Hz, 1H), 4.50 |
| | (d, J = 15.3 Hz, 1H), 4.23 (t, J = 11.2 Hz, 1H), 3.93 (td, J = 10.6, 4.3 Hz, |
| | 1H), 3.84 (s, 3H), 2.03 (s, 3H), 1.99-1.85 (m, 3H), 1.59 (s, 3H), 1.57-1.33 |
| | (m, 5H), 1.31-1.22 (m, 1H), 1.21-1.11 (m, 1H), 1.10-0.85 (m, 3H), 0.75- |
| | 0.38 (m, 11H). |
| 302 | ¹H NMR (400 MHz, DMSO-d₆) δ 13.40-11.44 (broad m, 1H), 8.56 (s, |
| | 1H), 8.28 (d, J = 2.5 Hz, 1H), 7.93 (br s, 1H), 7.67 (br s, 2H), 7.48-7.39 |
| | (m, 2H), 7.30 (t, J = 7.6 Hz, 1H), 7.13 (dd, J = 12.3, 7.6 Hz, 2H), 5.27 (dd, |
| | J = 11.0, 4.3 Hz, 1H), 4.87 (d, J = 15.4 Hz, 1H), 4.49 (d, J = 15.4 Hz, 1H), |
| | 4.28 (t, J = 11.2 Hz, 1H), 3.99 (td, J = 12.2, 10.5, 4.6 Hz, 1H), 3.84 (s, 3H), |
| | 2.29-2.11 (m, 2H), 1.87 (dd, J = 15.2, 8.8 Hz, 1H), 1.76 (s, 3H), 1.59 (br |
| | s, 6H), 1.48 (d, J = 12.7 Hz, 2H), 1.44-1.32 (m, 2H), 1.18-1.03 (m, 3H), |
| | 0.88-0.72 (m, 2H), 0.52 (s, 9H). |
| 303 | ¹H NMR (400 MHz, DMSO-d₆) δ 13.27-11.40 (broad m, 1H), 8.69 (s, |
| | 1H), 8.30 (s, 2H), 7.89 (s, 1H), 7.65 (s, 2H), 7.37-7.22 (m, 1H), 7.17 (d, |
| | J = 7.6 Hz, 1H), 7.09 (d, J = 7.7 Hz, 1H), 5.43 (d, J = 8.2 Hz, 1H), 4.86 (d, |
| | J = 16.2 Hz, 1H), 4.57 (d, J = 16.3 Hz, 1H), 4.12 (t, J = 11.1 Hz, 1H), 3.95 |
| | (td, J = 9.6, 9.0, 5.3 Hz, 1H), 2.95 (s, 6H), 2.04 (s, 3H), 1.98-1.81 (m, 3H), |
| | 1.58 (s, 3H), 1.56-1.11 (m, 7H), 1.11-0.85 (m, 3H), 0.76-0.38 (m, 11H). |
| 304 | ¹H NMR (400 MHz, DMSO-d₆) δ 13.50-11.20 (broad m, 1H), 8.71 (s, |
| | 1H), 8.29 (s, 2H), 7.93 (s, 1H), 7.66 (s, 2H), 7.30 (s, 1H), 7.13 (t, J = 9.5 |
| | Hz, 2H), 5.43 (s, 1H), 4.88 (d, J = 16.3 Hz, 1H), 4.56 (d, J = 16.3 Hz, 1H), |
| | 4.16 (t, J = 11.2 Hz, 1H), 4.08-3.94 (m, 1H), 2.95 (s, 6H), 2.31-2.09 (m, |
| | 2H), 1.91-1.69 (m, 4H), 1.59 (s, 6H), 1.53-1.44 (m, 2H), 1.44-1.32 (m, |
| | 2H), 1.20-1.01 (m, 3H), 0.87-0.74 (m, 2H), 0.55 (s, 9H). |
| 305 | ¹H NMR (400 MHz, DMSO-d₆) δ 13.72-12.18 (broad m, 1H), 8.69 (s, |
| | 1H), 8.50 (s, 2H), 7.89 (d, J = 6.7 Hz, 1H), 7.72-7.55 (m, 2H), 7.30 (t, |
| | J = 7.6 Hz, 1H), 7.18 (d, J = 7.6 Hz, 1H), 7.09 (d, J = 7.7 Hz, 1H), 5.43 (dd, |
| | J = 10.7, 4.3 Hz, 1H), 4.85 (d, J = 16.4 Hz, 1H), 4.62 (d, J = 16.5 Hz, 1H), |
| | 4.17 (t, J = 11.1 Hz, 1H), 3.95 (overlapped with water, dt, J = 10.8, 5.2 Hz, |
| | 1H), 3.78-3.71 (overlapped with water, m, 4H), 3.30-3.16 (m, 4H), 2.04 |
| | (s, 3H), 1.98-1.79 (m, 3H), 1.59 (s, 3H), 1.56-0.86 (m, 11H), 0.60 (s, |
| | 9H), 0.55-0.48 (m, 1H). |
| 306 | ¹H NMR (400 MHz, DMSO-d₆) δ 13.23-11.55 (broad m, 1H), 8.71 (s, |
| | 1H), 8.50 (s, 2H), 7.93 (s, 1H), 7.66 (s, 2H), 7.30 (s, 1H), 7.20-7.03 (m, |
| | 2H), 5.43 (s, 1H), 4.87 (d, J = 16.4 Hz, 1H), 4.61 (d, J = 16.5 Hz, 1H), 4.20 |
| | (t, J = 11.2 Hz, 1H), 4.09-3.95 (m, 1H), 3.75 (dd, J = 6.1, 3.6 Hz, 4H), |
| | 3.27-3.16 (m, 4H), 2.30-2.11 (m, 2H), 1.90-1.70 (m, 4H), 1.68-1.29 |
| | (m, 10H), 1.20-1.00 (m, 3H), 0.90-0.72 (m, 2H), 0.55 (s, 9H). |
| 311 | ¹H NMR (400 MHz, DMSO-d₆) δ 13.28-11.51 (broad m, 1H), 8.54 (br s, |
| | 1H), 7.91 (br s, 2H), 7.66 (br s, 2H), 7.30 (br s, 1H), 7.12 (br s, 2H), 5.16 |
| | (br s, 1H), 3.90 (br s, 1H), 3.29 (s, 1H), 2.29-2.10 (m, 2H), 1.85-1.30 (m, |
| | 14H), 1.21-0.99 (m, 3H), 0.87-0.71 (m, 2H), 0.52 (s, 9H). |
| 312 | ¹H NMR (400 MHz, DMSO-d₆) δ 13.19-11.61 (broad m, 1H), 8.49 (br s, |
| | 1H), 7.89 (br s, 2H), 7.63 (br s, 2H), 7.38-6.94 (m, 3H), 5.16 (br s, 1H), |
| | 3.87 (t, J = 10.9 Hz, 1H), 3.27-3.14 (m, 1H), 2.16-1.98 (m, 3H), 1.93 (br |
| | s, 2H), 1.68-1.32 (m, 9H), 1.25 (br s, 2H), 1.07-0.87 (m, 3H), 0.68-0.43 |
| | (m, 11H). ¹H NMR (400 MHz, DMSO-d₆ + 10% D2O) δ 8.48 (s, 1H), 7.85 |
| | (d, J = 7.7 Hz, 1H), 7.70-7.48 (m, 2H), 7.26 (s, 1H), 7.14 (d, J = 7.5 Hz, |
| | 1H), 7.06 (d, J = 7.6 Hz, 1H), 5.20-4.97 (m, 1H), 4.01-3.71 (m, 1H), 3.39- |
| | 3.09 (br m, 1H), 2.11-1.80 (m, 5H), 1.58-1.30 (m, 9H), 1.29-1.06 (m, |
| | 2H), 1.05-0.78 (m, 3H), 0.61-0.42 (m, 11H). |
| 313 | ¹H NMR (400 MHz, DMSO-d₆) δ 13.05 (s, 1H), 8.69 (d, J = 1.5 Hz, 1H), |
| | 8.59 (d, J = 1.5 Hz, 2H), 7.93 (s, 1H), 7.66 (s, 2H), 7.31 (s, 1H), 7.23-7.05 |
| | (m, 2H), 5.36 (s, 1H), 4.87 (d, J = 15.8 Hz, 1H), 4.63 (d, J = 15.8 Hz, 1H), |
| | 4.38 (s, 1H), 4.04 (s, 1H), 3.96 (dt, J = 11.1, 3.2 Hz, 2H), 3.46 (dddd, J = |
| | 11.4, 6.9, 5.5, 1.8 Hz, 2H), 3.05 (ddd, J = 15.6, 9.3, 7.1 Hz, 1H), 2.22 (d, |
| | J = 7.5 Hz, 2H), 2.02-1.66 (m, 9H), 1.61 (s, 3H), 1.39 (dd, J = 17.8, 13.1 |
| | Hz, 1H), 0.81 (dt, J = 17.9, 7.3 Hz, 6H), 0.54 (s, 9H). |
| 314 | ¹H NMR (400 MHz, DMSO-d₆) δ 13.02 (s, 1H), 8.69 (d, J = 1.5 Hz, 1H), |
| | 8.59 (d, J = 1.5 Hz, 1H), 8.53 (s, 1H), 7.91 (s, 1H), 7.66 (s, 2H), 7.31 (s, |
| | 1H), 7.16 (d, J = 11.5 Hz, 2H), 5.34 (s, 1H), 4.86 (d, J = 15.9 Hz, 1H), 4.65 |
| | (d, J = 15.9 Hz, 1H), 4.41 (t, J = 10.9 Hz, 1H), 3.96 (dt, J = 11.1, 3.3 Hz, |
| | 3H), 3.53-3.38 (m, 2H), 3.05 (p, J = 7.8 Hz, 1H), 2.21 (d, J = 7.6 Hz, 2H), |
| | 1.90-1.71 (m, 9H), 1.59 (s, 3H), 1.37-1.30 (m, 1H), 1.24 (s, 2H), 0.79 (d, |
| | J = 7.7 Hz, 5H), 0.70 (d, J = 6.5 Hz, 3H), 0.22 (s, 3H). |
| 315 | ¹H NMR (400 MHz, DMSO-d₆) δ 13.02 (s, 1H), 8.69 (d, J = 1.5 Hz, 1H), |
| | 8.60 (d, J = 1.4 Hz, 1H), 8.51 (s, 1H), 7.89 (s, 1H), 7.65 (s, 2H), 7.31 (s, |
| | 1H), 7.20 (s, 1H), 7.11 (s, 1H), 5.34 (d, J = 9.7 Hz, 1H), 4.84 (d, J = 15.8 |
| | Hz, 1H), 4.66 (d, J = 15.8 Hz, 1H), 4.41 (s, 1H), 4.02-3.87 (m, 3H), 3.54- |
| | 3.40 (m, 2H), 3.05 (p, J = 7.9 Hz, 1H), 2.09-1.95 (m, 4H), 1.79 (dd, J = |
| | 7.2, 3.9 Hz, 5H), 1.58 (s, 3H), 1.19 (d, J = 32.3 Hz, 4H), 0.70 (d, J = 6.6 |
| | Hz, 3H), 0.60 (d, J = 6.5 Hz, 3H), 0.54 (d, J = 6.6 Hz, 3H), 0.23 (d, J = 6.3 |
| | Hz, 3H). |
| 316 | ¹H NMR (400 MHz, DMSO-d₆) δ 13.01 (s, 1H), 8.63 (s, 1H), 8.52 (s, 2H), |
| | 7.91 (s, 1H), 7.64 (s, 2H), 7.31 (s, 1H), 7.15 (s, 2H), 5.40 (s, 1H), 4.91 (d, |
| | J = 16.5 Hz, 1H), 4.81 (p, J = 6.0 Hz, 1H), 4.63 (d, J = 16.5 Hz, 1H), 4.25 |
| | (t, J = 11.1 Hz, 1H), 4.00 (s, 1H), 2.21 (d, J = 7.6 Hz, 2H), 1.76 (dd, J = |
| | 23.0, 10.4 Hz, 6H), 1.57 (s, 4H), 1.31 (d, J = 1.9 Hz, 3H), 1.30 (d, J = 1.9 |
| | Hz, 3H), 0.83-0.69 (m, 9H), 0.24 (s, 3H). |
| 317 | ¹H NMR (400 MHz, DMSO-d₆) δ 13.01 (s, 1H), 8.62 (s, 1H), 8.53 (s, 2H), |
| | 7.90 (s, 1H), 7.64 (s, 2H), 7.31 (s, 1H), 7.19 (d, J = 7.5 Hz, 1H), 7.11 (d, |
| | J = 7.4 Hz, 1H), 5.39 (d, J = 9.6 Hz, 1H), 4.89 (d, J = 16.5 Hz, 1H), 4.81 (h, |
| | J = 6.0 Hz, 1H), 4.64 (d, J = 16.6 Hz, 1H), 4.24 (d, J = 11.3 Hz, 1H), 3.95 |
| | (s, 1H), 2.12-1.86 (m, 6H), 1.54 (d, J = 24.4 Hz, 4H), 1.31 (d, J = 2.3 Hz, |
| | 3H), 1.30 (d, J = 2.3 Hz, 3H), 1.24 (d, J = 3.7 Hz, 2H), 0.75 (d, J = 6.6 Hz, |
| | 3H), 0.60 (d, J = 6.5 Hz, 3H), 0.55 (d, J = 6.5 Hz, 3H), 0.26 (d, J = 6.4 Hz, 3H). |
| 318 | ¹H NMR (400 MHz, DMSO-d₆) δ 13.07 (s, 1H), 8.69 (d, J = 1.5 Hz, 1H), |
| | 8.60 (d, J = 1.5 Hz, 2H), 7.95 (s, 1H), 7.67 (s, 2H), 7.37 (s, 1H), 7.26 (d, |
| | J = 7.8 Hz, 1H), 7.19 (d, J = 7.5 Hz, 1H), 5.35 (d, J = 9.7 Hz, 1H), 4.86 (d, |
| | J = 15.7 Hz, 1H), 4.63 (d, J = 15.7 Hz, 1H), 4.42 (t, J = 11.2 Hz, 1H), 4.11 |
| | (d, J = 12.1 Hz, 1H), 3.96 (dt, J = 11.0, 3.2 Hz, 2H), 3.46 (dddd, J = 11.3, |
| | 7.0, 5.1, 1.7 Hz, 2H), 3.04 (p, J = 7.8, 7.3 Hz, 1H), 2.16-1.99 (m, 4H), |
| | 1.93-1.72 (m, 5H), 1.62 (s, 3H), 1.42-1.36 (m, 1H), 0.95 (dd, J = 6.7, 2.1 |
| | Hz, 6H), 0.52 (s, 9H). |
| 319 | ¹H NMR (400 MHz, DMSO-d₆) δ 13.08 (s, 1H), 8.69 (d, J = 1.5 Hz, 1H), |
| | 8.59 (d, J = 1.5 Hz, 2H), 7.93 (s, 1H), 7.34 (d, J = 21.7 Hz, 2H), 7.13 (s, |
| | 1H), 5.35 (s, 1H), 4.85 (d, J = 15.7 Hz, 1H), 4.63 (d, J = 15.7 Hz, 1H), 4.40 |
| | (s, 1H), 4.05 (s, 1H), 3.96 (dt, J = 11.0, 3.3 Hz, 2H), 3.53-3.39 (m, 2H), |
| | 3.05 (p, J = 8.0, 7.4 Hz, 1H), 1.91-1.68 (m, 8H), 1.61 (s, 3H), 1.45-1.37 |
| | (m, 1H), 1.33-1.21 (m, 2H), 1.21-1.03 (m, 7H), 0.54 (s, 9H). |

### Example 80: Compounds 321 to 330

Compounds 321 to 330, depicted in Table 19, can be prepared following the procedures described above for compounds 1-320 and CFTR modulating activity can be assessed using one or more of the assays outlined below.

TABLE 19
Compounds 321 - 330

| Compound Number | Structure |
|---|---|
| 321 | 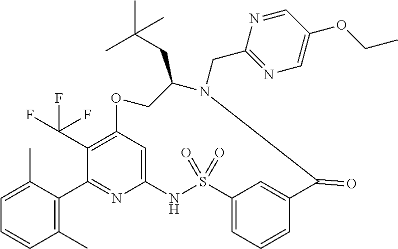 [SMILES: CCOc1cnc(CN2C(=O)c3cccc(c3)S(=O)(=O)Nc3cc(c(C(F)(F)F)c(-c4c(C)cccc4C)n3)OC[C@H]2CC(C)(C)C)nc1] |
| 322 | 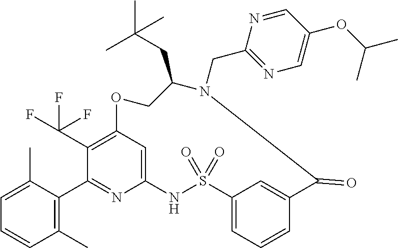 [SMILES: Cc1cccc(C)c1-c1nc2cc(c1C(F)(F)F)OC[C@@H](CC(C)(C)C)N(Cc1ncc(OC(C)C)cn1)C(=O)c1cccc(c1)S(=O)(=O)N2] |
| 323 | 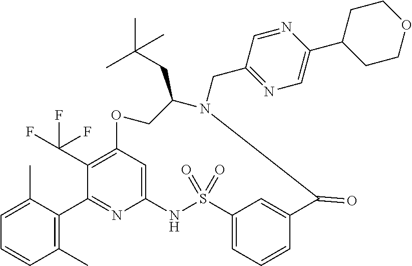 [SMILES: Cc1cccc(C)c1-c1nc2cc(c1C(F)(F)F)OC[C@@H](CC(C)(C)C)N(Cc1cnc(C3CCOCC3)cn1)C(=O)c1cccc(c1)S(=O)(=O)N2] |
| 324 | 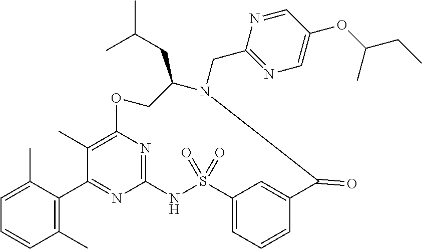 [SMILES: CCC(C)Oc1cnc(CN2C(=O)c3cccc(c3)S(=O)(=O)Nc3nc(c(C)c(-c4c(C)cccc4C)n3)OC[C@H]2CC(C)C)nc1] |
| 325 | 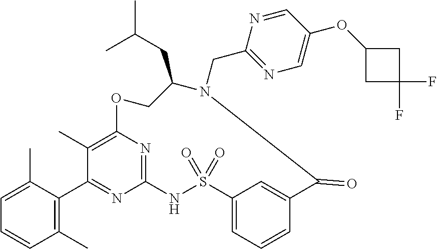 [SMILES: Cc1cccc(C)c1-c1nc2nc(c1C)OC[C@@H](CC(C)C)N(Cc1ncc(OC3CC(F)(F)C3)cn1)C(=O)c1cccc(c1)S(=O)(=O)N2] |
| 326 | 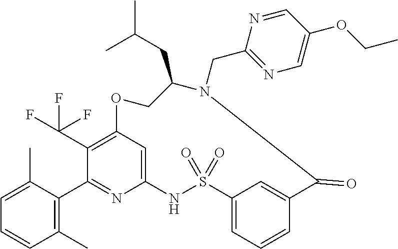 [SMILES: CCOc1cnc(CN2C(=O)c3cccc(c3)S(=O)(=O)Nc3cc(c(C(F)(F)F)c(-c4c(C)cccc4C)n3)OC[C@H]2CC(C)C)nc1] |
| 327 | 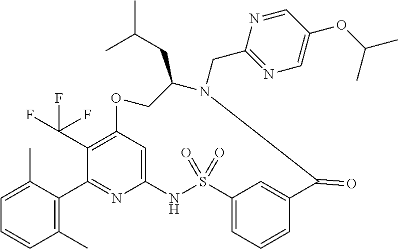 [SMILES: Cc1cccc(C)c1-c1nc2cc(c1C(F)(F)F)OC[C@@H](CC(C)C)N(Cc1ncc(OC(C)C)cn1)C(=O)c1cccc(c1)S(=O)(=O)N2] |
| 328 | 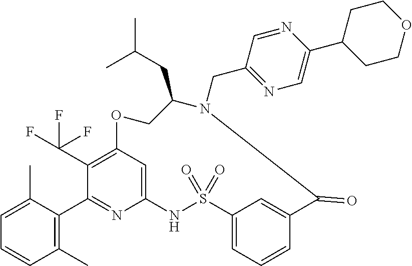 [SMILES: Cc1cccc(C)c1-c1nc2cc(c1C(F)(F)F)OC[C@@H](CC(C)C)N(Cc1cnc(C3CCOCC3)cn1)C(=O)c1cccc(c1)S(=O)(=O)N2] |
| 329 | 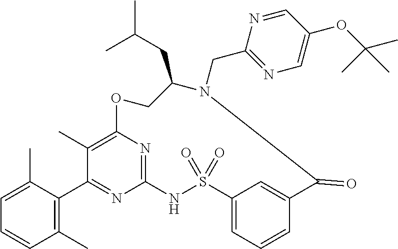 [SMILES: Cc1cccc(C)c1-c1nc2nc(c1C)OC[C@@H](CC(C)C)N(Cc1ncc(OC(C)(C)C)cn1)C(=O)c1cccc(c1)S(=O)(=O)N2] |
| 330 | 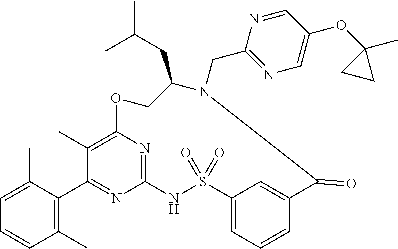 [SMILES: Cc1cccc(C)c1-c1nc2nc(c1C)OC[C@@H](CC(C)C)N(Cc1ncc(OC3(C)CC3)cn1)C(=O)c1cccc(c1)S(=O)(=O)N2] |

### VI. Bioactivity

### A. 3T3 Assay

### 1. Membrane Potential Optical Methods for Assaying F508del Modulation Properties of Compounds

The assay utilizes fluorescent voltage sensing dyes to measure changes in membrane potential using a fluorescent plate reader (e.g., FLIPR III, Molecular Devices, Inc.) as a readout for increase in functional F508del in NIH 3T3 cells. The driving force for the response is the creation of a chloride ion gradient in conjunction with channel activation by a single liquid addition step after the cells have previously been treated with compounds and subsequently loaded with a voltage sensing dye.

### 2. Identification of Corrector Compounds

To identify correctors of F508del, a single-addition HTS assay format was developed. This HTS assay utilizes fluorescent voltage sensing dyes to measure changes in membrane potential on the FLIPR III as a measurement for increase in gating (conductance) of F508del in F508del NIH 3T3 cells. The F508del NIH 3T3 cell cultures were incubated with the corrector compounds at a range of concentrations for 18-24 hours at 37° C., and subsequently loaded with a redistribution dye. The driving force for the response is a Cl⁻ ion gradient in conjunction with channel activation with forskolin in a single liquid addition step using a fluorescent plate reader such as FLIPR III. The efficacy and potency of the putative F508del correctors was compared to that of the known corrector, lumacaftor, in combination with acutely added 300 nM Ivacaftor.

### 3. Solutions

Bath Solution #1: (in mM) NaCl 160, KCl 4.5, CaCl₂) 2, MgCl₂ 1, HEPES 10, pH 7.4 with NaOH.

Chloride-free bath solution: Chloride salts in Bath Solution #1 (above) are substituted with gluconate salts.

### 4. Cell Culture

NIH3T3 mouse fibroblasts stably expressing F508del are used for optical measurements of membrane potential. The cells are maintained at 37° C. in 5% CO₂ and 90% humidity in Dulbecco's modified Eagle's medium supplemented with 2 mM glutamine, 10% fetal bovine serum, 1×NEAA, b-ME, 1× pen/strep, and 25 mM HEPES in 175 cm² culture flasks. For all optical assays, the cells were seeded at ˜20,000/well in 384-well matrigel-coated plates. For the correction assays, the cells are cultured at 37° C. with and without compounds for 16-24 hours.

### B. Enteroid Assay

### 1. Solutions

Base medium (ADF+++) consisted of Advanced DMEM/Ham's F12, 2 mM Glutamax, 10 mM HEPES, 1 pg/mL penicillin/streptomycin.

Intestinal enteroid maintenance medium (IEMM) consisted of ADF+++, 1× B27 supplement, 1× N₂ supplement, 1.25 mM N-acetyl cysteine, 10 mM Nicotinamide, 50 ng/mL hEGF, 10 nM Gastrin, 1 pg/mL hR-spondin-1, 100 ng/mL hNoggin, TGF-b type 1 inhibitor A-83-01, 100 μg/mL Primocin, 10 pM P38 MAPK inhibitor SB202190.

Bath 1 Buffer consisted of 1 mM MgCl₂, 160 mM NaCl, 4.5 mM KCl, 10 mM HEPES, 10 mM Glucose, 2 mM CaCl₂).

Chloride Free Buffer consisted of 1 mM Magnesium Gluconate, 2 mM Calcium Gluconate, 4.5 mM Potassium Gluconate, 160 mM Sodium Gluconate, 10 mM HEPES, 10 mM Glucose.

Bath1 Dye Solution consisted of Bath 1 Buffer, 0.04% Pluronic F127, 20 pM Methyl Oxonol, 30 μM CaCCinh-A01, 30 μM Chicago Sky Blue.

Chloride Free Dye Solution consisted of Chloride Free Buffer, 0.04% Pluronic F127, 20 μM Methyl Oxonol, 30 μM CaCCinh-A01, 30 μM Chicago Sky Blue.

Chloride Free Dye Stimulation Solution consisted of Chloride Free Dye Solution, 10 μM forskolin, 100 μM IBMX, and 300 nM Compound III.

### 2. Cell Culture

Human intestinal epithelial enteroid cells were obtained from the Hubrecht Institute for Developmental Biology and Stem Cell Research, Utrecht, The Netherlands and expanded in T-Flasks as previously described (Dekkers J F, Wiegerinck C L, de Jonge H R, Bronsveld I, Janssens H M, de Winter-de Groot K M, Brandsma A M, de Jong N W M, Bijvelds M J C, Scholte B J, Nieuwenhuis E E S, van den Brink S, Clevers H, van der Ent C K, Middendorp S and M Beekman J M. A functional CFTR assay using primary cystic fibrosis intestinal organoids. Nat Med. 2013 July; 19(7):939-45.).

### 3. Enteroid Cell Harvesting and Seeding

Cells were recovered in cell recovery solution, collected by centrifugation at 650 rpm for 5 min at 4° C., resuspended in TryPLE and incubated for 5 min at 37° C. Cells were then collected by centrifugation at 650 rpm for 5 min at 4° C. and resuspended in IEMM containing 10 μM ROCK inhibitor (RI). The cell suspension was passed through a 40 μm cell strainer and resuspended at 1×106 cells/mL in IEMM containing 10 μM RI. Cells were seeded at 5000 cells/well into multi-well plates and incubated for overnight at 37° C., 95% humidity and 5% CO₂ prior to assay.

### 4. Membrane Potential Dye, Enteroid Assay A

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added, and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 3 μM (S)—N-((6-aminopyridin-2-yl)sulfonyl)-6-(3-fluoro-5-isobutoxyphenyl)-2-(2,2,4-trimethylpyrrolidin-1-yl)nicotinamide, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

### 5. Membrane Potential Dye, Enteroid Assay B

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 1 μM (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

### C. HBE Assay

### 1. Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HIPO₄, 3.3 KH₂PO₄, 1.2 MgCl₂, 1.2 CaCl₂), 10 Glucose, 10 HEPES (pH adjusted to 7.4 with NaOH) and the apical solution contained (in mM) 145 NaGluconate, 1.2 MgCl₂, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

### 2. Identification of Corrector Compounds

The activity of the CFTR corrector compounds on the CFTR-mediated I_{SC} was determined in Ussing chamber studies as described above. The F508del/MF-HBE cell cultures were either incubated with the corrector compounds at a range of concentrations in combination with 1 μM Ivacaftor or were incubated with the corrector compounds at a single fixed concentration of 10 μM in combination with 1 μM Ivacaftor for 18-24 hours at 37° C. and in the presence of 20% human serum. The concentration of corrector compounds with 1 μM Ivacaftor during the 18-24 hours incubations was kept constant throughout the Ussing chamber measurement of the CFTR-mediated I_{SC} to ensure compounds were present throughout the entire experiment. The efficacy and potency of the putative F508del correctors was compared to that of the known Vertex corrector, (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, in combination with 18 μM Tezacaftor and 1 μM Ivacaftor.

### Biological Activity Data

Table 20 represents CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” % Activity: +++ is >60%; ++ is 30-60%; + is <30%).

TABLE 20
Biological Activity of Compounds

| | | | 3T3 | Ent. | Ent. A | Ent. | Ent. B |
|---|---|---|---|---|---|---|---|
| | | 3T3 | Max | A | Max | B | Max |
| | | EC₅₀ | Activity | EC₅₀ | Activity | EC₅₀ | Activity |
| No. | Structure | (μM) | (%) | (μM) | (%) | (μM) | (%) |
| 1 | 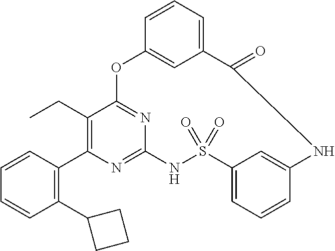 [SMILES: CCc1c2nc(nc1-c1ccccc1C1CCC1)NS(=O)(=O)c1cccc(c1)NC(=O)c1cccc(c1)O2] | +++ | +++ | +++ | ++ | | |
| 2 | 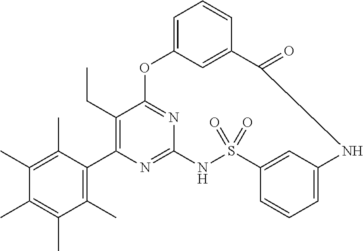 [SMILES: CCc1c2nc(nc1-c1c(C)c(C)c(C)c(C)c1C)NS(=O)(=O)c1cccc(c1)NC(=O)c1cccc(c1)O2] | +++ | +++ | | | | |
| 3 | 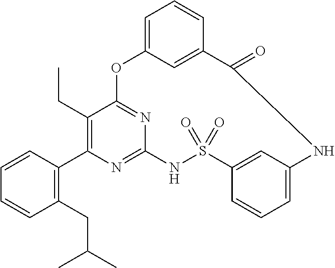 [SMILES: CCc1c2nc(nc1-c1ccccc1CC(C)C)NS(=O)(=O)c1cccc(c1)NC(=O)c1cccc(c1)O2] | +++ | +++ | +++ | +++ | | |
| 4 | 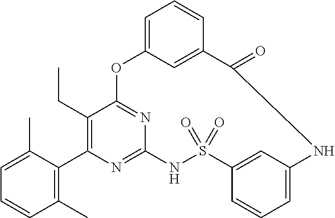 [SMILES: CCc1c2nc(nc1-c1c(C)cccc1C)NS(=O)(=O)c1cccc(c1)NC(=O)c1cccc(c1)O2] | +++ | +++ | ++ | +++ | | |
| 5 | 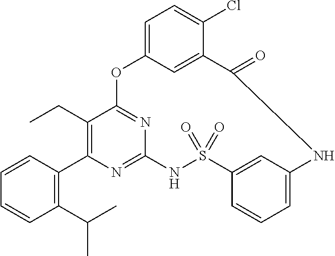 [SMILES: CCc1c2nc(nc1-c1ccccc1C(C)C)NS(=O)(=O)c1cccc(c1)NC(=O)c1cc(ccc1Cl)O2] | +++ | +++ | +++ | +++ | | |
| 6 | 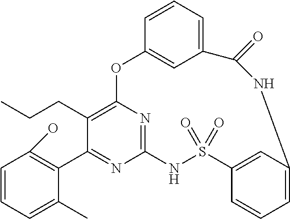 [SMILES: CCCc1c2nc(nc1-c1c(C)cccc1O)NS(=O)(=O)c1cccc(c1)NC(=O)c1cccc(c1)O2] | | | ++ | ++ | | |
| 7 | 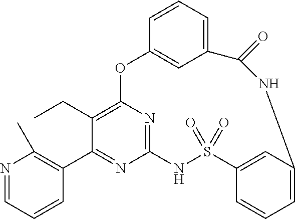 [SMILES: CCc1c2nc(nc1-c1cccnc1C)NS(=O)(=O)c1cccc(c1)NC(=O)c1cccc(c1)O2] | | | ND | + | | |
| 8 | 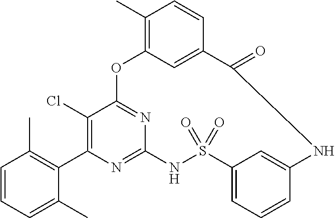 [SMILES: Cc1ccc2cc1Oc1nc(nc(-c3c(C)cccc3C)c1Cl)NS(=O)(=O)c1cccc(c1)NC2=O] | +++ | +++ | | | | |
| 9 | 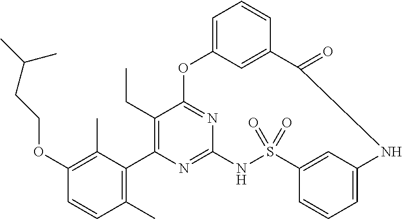 [SMILES: CCc1c2nc(nc1-c1c(C)ccc(OCCC(C)C)c1C)NS(=O)(=O)c1cccc(c1)NC(=O)c1cccc(c1)O2] | | | +++ | ++ | | |
| 10 | 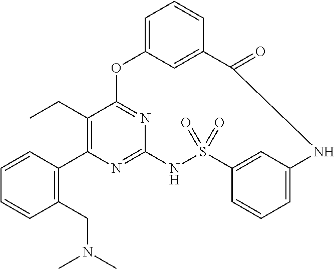 [SMILES: CCc1c2nc(nc1-c1ccccc1CN(C)C)NS(=O)(=O)c1cccc(c1)NC(=O)c1cccc(c1)O2] | ND | + | | | | |
| 11 | 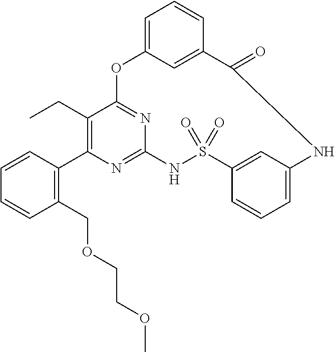 [SMILES: CCc1c2nc(nc1-c1ccccc1COCCOC)NS(=O)(=O)c1cccc(c1)NC(=O)c1cccc(c1)O2] | +++ | +++ | | | | |
| 12 | 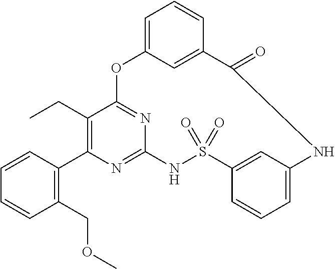 [SMILES: CCc1c2nc(nc1-c1ccccc1COC)NS(=O)(=O)c1cccc(c1)NC(=O)c1cccc(c1)O2] | +++ | +++ | | | | |
| 13 | 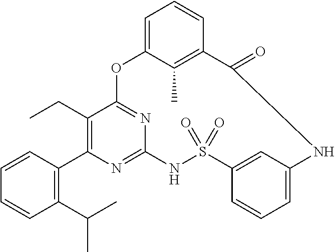 | +++ | +++ | | | | |
| 14 | 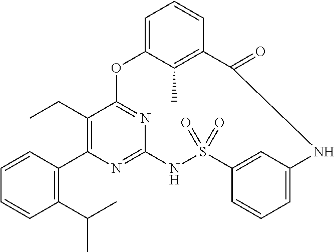 | +++ | +++ | +++ | +++ | | |
| 15 | 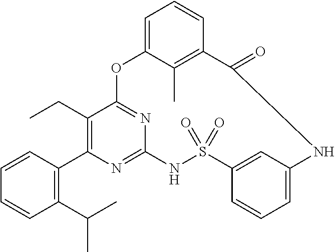 [SMILES: CCc1c2nc(nc1-c1ccccc1C(C)C)NS(=O)(=O)c1cccc(c1)NC(=O)c1cccc(c1C)O2] | +++ | +++ | +++ | +++ | | |
| 16 | 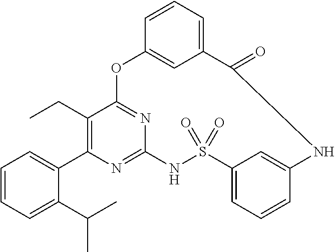 [SMILES: CCc1c2nc(nc1-c1ccccc1C(C)C)NS(=O)(=O)c1cccc(c1)NC(=O)c1cccc(c1)O2] | +++ | +++ | +++ | +++ | | |
| 17 | 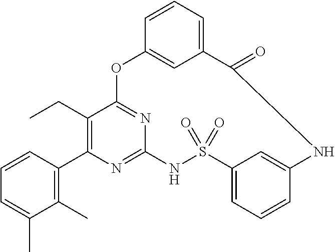 [SMILES: CCc1c2nc(nc1-c1cccc(C)c1C)NS(=O)(=O)c1cccc(c1)NC(=O)c1cccc(c1)O2] | +++ | +++ | | | | |
| 18 | 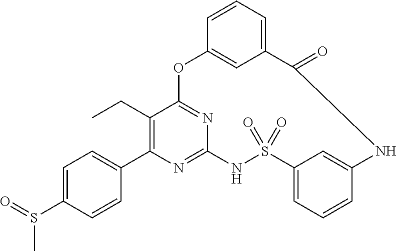 [SMILES: CCc1c2nc(nc1-c1ccc(S(C)=O)cc1)NS(=O)(=O)c1cccc(c1)NC(=O)c1cccc(c1)O2] | ND | + | | | | |
| 19 | 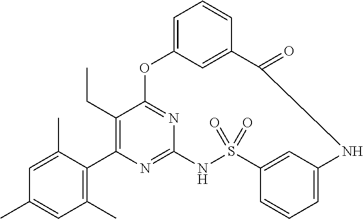 [SMILES: CCc1c2nc(nc1-c1c(C)cc(C)cc1C)NS(=O)(=O)c1cccc(c1)NC(=O)c1cccc(c1)O2] | +++ | +++ | | | | |
| 20 | 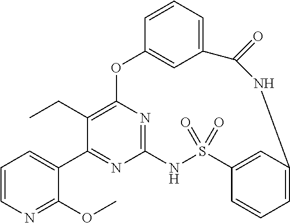 [SMILES: CCc1c2nc(nc1-c1cccnc1OC)NS(=O)(=O)c1cccc(c1)NC(=O)c1cccc(c1)O2] | | | ND | + | | |
| 21 | 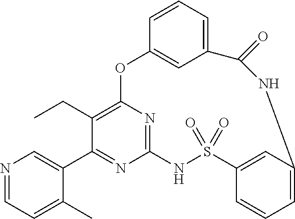 [SMILES: CCc1c2nc(nc1-c1cnccc1C)NS(=O)(=O)c1cccc(c1)NC(=O)c1cccc(c1)O2] | | | ND | + | | |
| 22 | 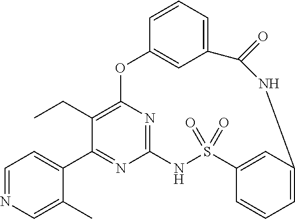 [SMILES: CCc1c2nc(nc1-c1ccncc1C)NS(=O)(=O)c1cccc(c1)NC(=O)c1cccc(c1)O2] | | | ND | + | | |
| 23 | 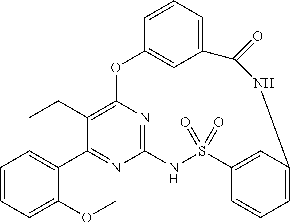 [SMILES: CCc1c2nc(nc1-c1ccccc1OC)NS(=O)(=O)c1cccc(c1)NC(=O)c1cccc(c1)O2] | | | + | ++ | | |
| 24 | 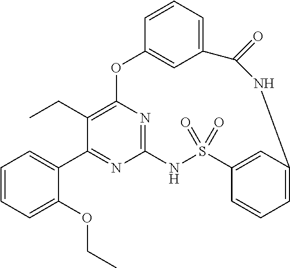 [SMILES: CCOc1ccccc1-c1nc2nc(c1CC)Oc1cccc(c1)C(=O)Nc1cccc(c1)S(=O)(=O)N2] | | | ++ | + | | |
| 25 | 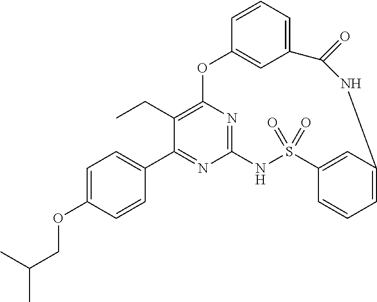 [SMILES: CCc1c2nc(nc1-c1ccc(OCC(C)C)cc1)NS(=O)(=O)c1cccc(c1)NC(=O)c1cccc(c1)O2] | | | ND | ND | | |
| 26 | 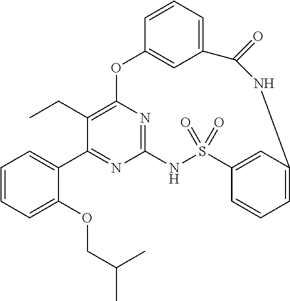 [SMILES: CCc1c2nc(nc1-c1ccccc1OCC(C)C)NS(=O)(=O)c1cccc(c1)NC(=O)c1cccc(c1)O2] | +++ | +++ | ++ | + | | |
| 27 | 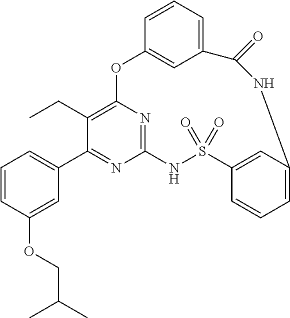 [SMILES: CCc1c2nc(nc1-c1cccc(OCC(C)C)c1)NS(=O)(=O)c1cccc(c1)NC(=O)c1cccc(c1)O2] | | | +++ | + | | |
| 28 | 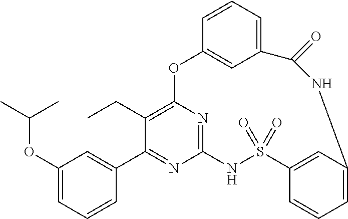 [SMILES: CCc1c2nc(nc1-c1cccc(OC(C)C)c1)NS(=O)(=O)c1cccc(c1)NC(=O)c1cccc(c1)O2] | | | ++ | + | | |
| 29 | 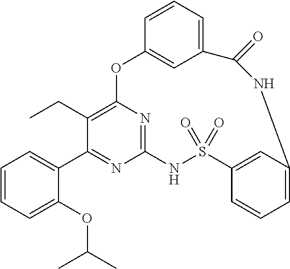 [SMILES: CCc1c2nc(nc1-c1ccccc1OC(C)C)NS(=O)(=O)c1cccc(c1)NC(=O)c1cccc(c1)O2] | +++ | +++ | ND | + | | |
| 30 | 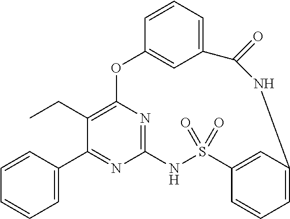 [SMILES: CCc1c2nc(nc1-c1ccccc1)NS(=O)(=O)c1cccc(c1)NC(=O)c1cccc(c1)O2] | | | ND | + | | |
| 31 | 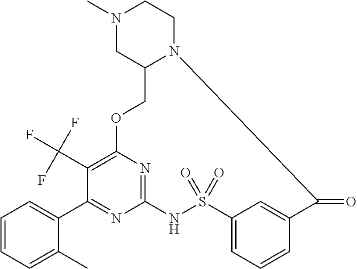 [SMILES: Cc1ccccc1-c1nc2nc(c1C(F)(F)F)OCC1CN(C)CCN1C(=O)c1cccc(c1)S(=O)(=O)N2] | | | + | ++ | | |
| 32 | 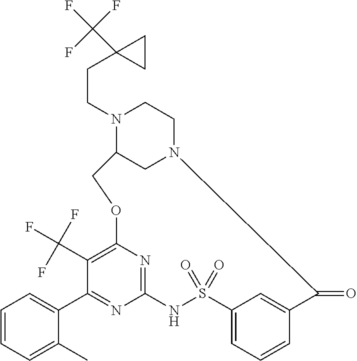 [SMILES: Cc1ccccc1-c1nc2nc(c1C(F)(F)F)OCC1CN(CCN1CCC1(C(F)(F)F)CC1)C(=O)c1cccc(c1)S(=O)(=O)N2] | | | ++ | ++ | | |
| 33 | 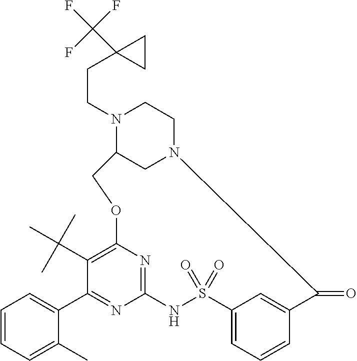 [SMILES: Cc1ccccc1-c1nc2nc(c1C(C)(C)C)OCC1CN(CCN1CCC1(C(F)(F)F)CC1)C(=O)c1cccc(c1)S(=O)(=O)N2] | | | ++ | +++ | | |
| 34 | 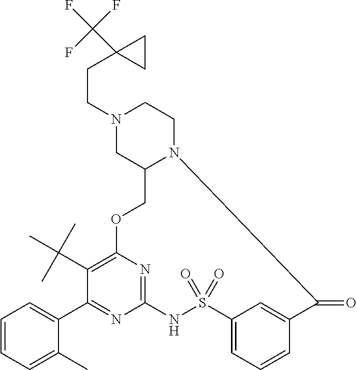 [SMILES: Cc1ccccc1-c1nc2nc(c1C(C)(C)C)OCC1CN(CCC3(C(F)(F)F)CC3)CCN1C(=O)c1cccc(c1)S(=O)(=O)N2] | | | ++ | ++ | | |
| 35 | 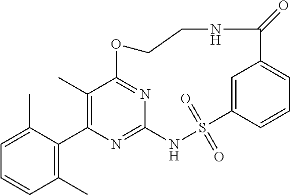 [SMILES: Cc1cccc(C)c1-c1nc2nc(c1C)OCCNC(=O)c1cccc(c1)S(=O)(=O)N2] | | | | | + | ++ |
| 36 | 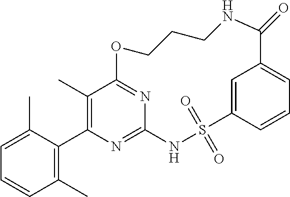 [SMILES: Cc1cccc(C)c1-c1nc2nc(c1C)OCCCNC(=O)c1cccc(c1)S(=O)(=O)N2] | | | | | + | + |
| 37 | 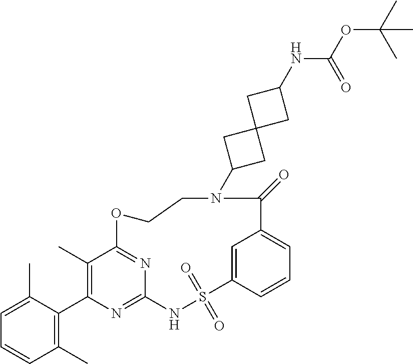 [SMILES: Cc1cccc(C)c1-c1nc2nc(c1C)OCCN(C1CC3(CC(NC(=O)OC(C)(C)C)C3)C1)C(=O)c1cccc(c1)S(=O)(=O)N2] | | | | | +++ | ++ |
| 38 | 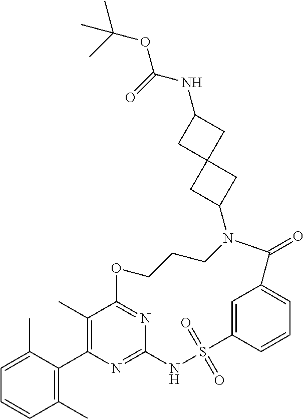 [SMILES: Cc1cccc(C)c1-c1nc2nc(c1C)OCCCN(C1CC3(CC(NC(=O)OC(C)(C)C)C3)C1)C(=O)c1cccc(c1)S(=O)(=O)N2] | | | | | +++ | + |
| 39 | 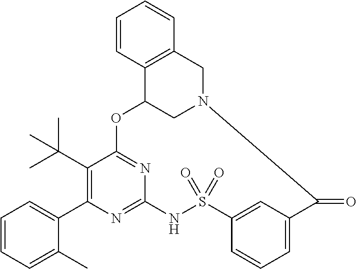 [SMILES: Cc1ccccc1-c1nc2nc(c1C(C)(C)C)OC1CN(Cc3ccccc31)C(=O)c1cccc(c1)S(=O)(=O)N2] | | | +++ | ++ | | |
| 40 | 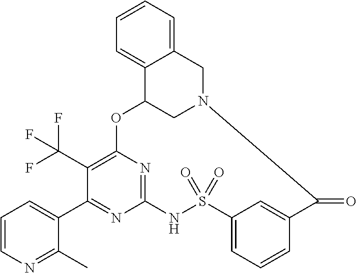 [SMILES: Cc1ncccc1-c1nc2nc(c1C(F)(F)F)OC1CN(Cc3ccccc31)C(=O)c1cccc(c1)S(=O)(=O)N2] | | | ND | + | | |
| 41 | 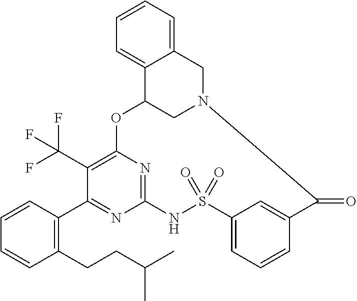 [SMILES: CC(C)CCc1ccccc1-c1nc2nc(c1C(F)(F)F)OC1CN(Cc3ccccc31)C(=O)c1cccc(c1)S(=O)(=O)N2] | | | +++ | +++ | | |
| 42 | 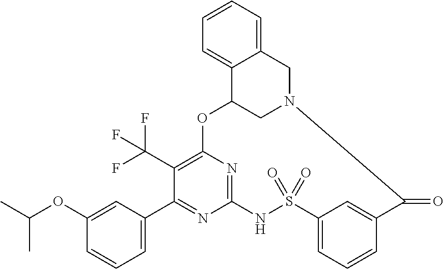 [SMILES: CC(C)Oc1cccc(-c2nc3nc(c2C(F)(F)F)OC2CN(Cc4ccccc42)C(=O)c2cccc(c2)S(=O)(=O)N3)c1] | | | +++ | + | | |
| 43 | 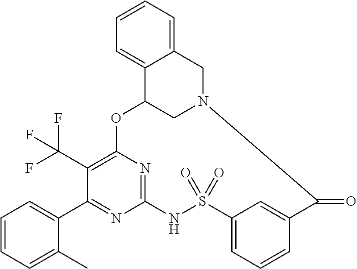 [SMILES: Cc1ccccc1-c1nc2nc(c1C(F)(F)F)OC1CN(Cc3ccccc31)C(=O)c1cccc(c1)S(=O)(=O)N2] | | | +++ | +++ | | |
| 44 | 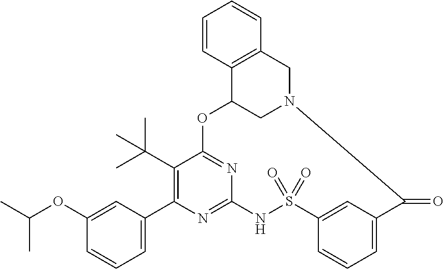 [SMILES: CC(C)Oc1cccc(-c2nc3nc(c2C(C)(C)C)OC2CN(Cc4ccccc42)C(=O)c2cccc(c2)S(=O)(=O)N3)c1] | | | ND | + | | |
| 45 | 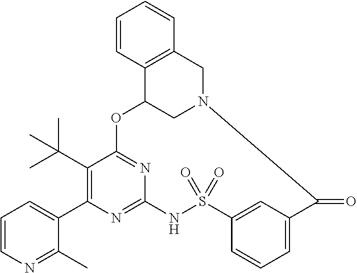 [SMILES: Cc1ncccc1-c1nc2nc(c1C(C)(C)C)OC1CN(Cc3ccccc31)C(=O)c1cccc(c1)S(=O)(=O)N2] | | | ND | + | | |
| 46 | 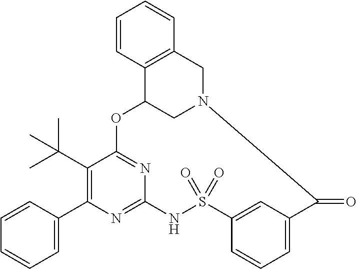 [SMILES: CC(C)(C)c1c2nc(nc1-c1ccccc1)NS(=O)(=O)c1cccc(c1)C(=O)N1Cc3ccccc3C(C1)O2] | | | ND | + | | |
| 47 | 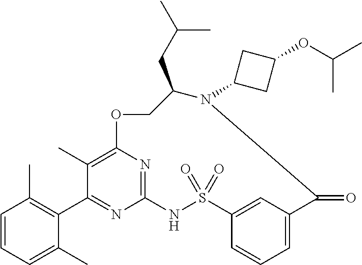 [SMILES: Cc1cccc(C)c1-c1nc2nc(c1C)OC[C@@H](CC(C)C)N([C@H]1C[C@@H](OC(C)C)C1)C(=O)c1cccc(c1)S(=O)(=O)N2] | | | | | +++ | ++ |
| 48 | 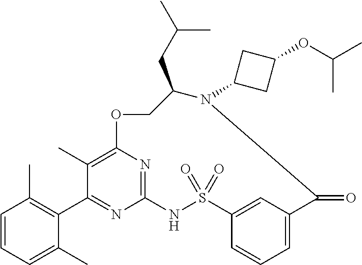 [SMILES: Cc1cccc(C)c1-c1nc2nc(c1C)OC[C@@H](CC(C)C)N([C@H]1C[C@@H](OC(C)C)C1)C(=O)c1cccc(c1)S(=O)(=O)N2] | | | | | +++ | +++ |
| 49 | 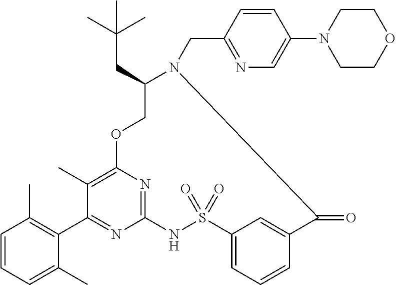 [SMILES: Cc1cccc(C)c1-c1nc2nc(c1C)OC[C@@H](CC(C)(C)C)N(Cc1ccc(N3CCOCC3)cn1)C(=O)c1cccc(c1)S(=O)(=O)N2] | See HBE assay data below | | | | | |
| 50 | 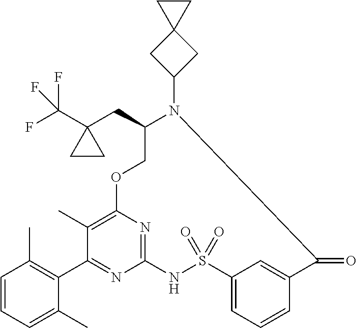 [SMILES: Cc1cccc(C)c1-c1nc2nc(c1C)OC[C@@H](CC1(C(F)(F)F)CC1)N(C1CC3(CC3)C1)C(=O)c1cccc(c1)S(=O)(=O)N2] | | | | | +++ | +++ |
| 51 | 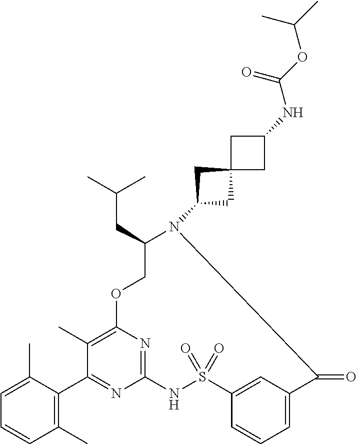 | | | | | +++ | +++ |
| 52 | 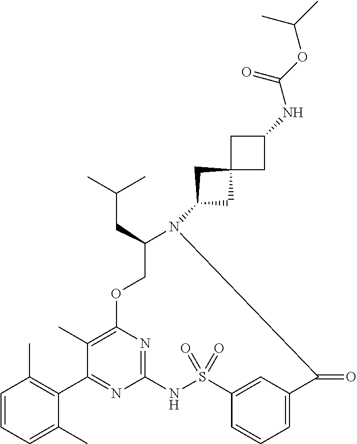 | | | | | +++ | +++ |
| 53 | 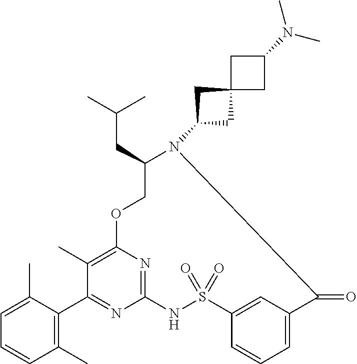 | | | | | +++ | +++ |
| 54 | 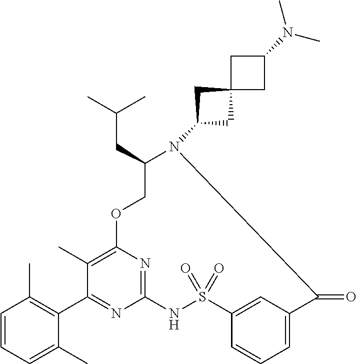 | | | | | +++ | +++ |
| 55 | 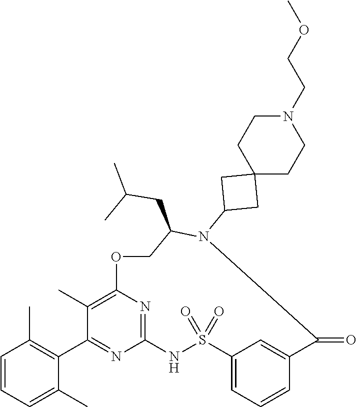 [SMILES: COCCN1CCC2(CC1)CC(N1C(=O)c3cccc(c3)S(=O)(=O)Nc3nc(c(C)c(-c4c(C)cccc4C)n3)OC[C@H]1CC(C)C)C2] | | | | | +++ | +++ |
| 56 | | See HBE assay data below | | | | | |
| 57 | 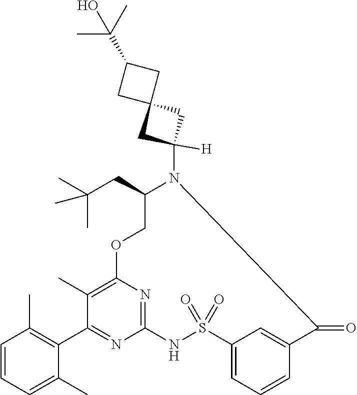 | | | | | +++ | +++ |
| 58 | 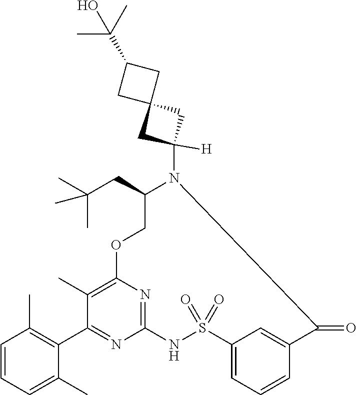 | | | | | +++ | +++ |
| 59 | 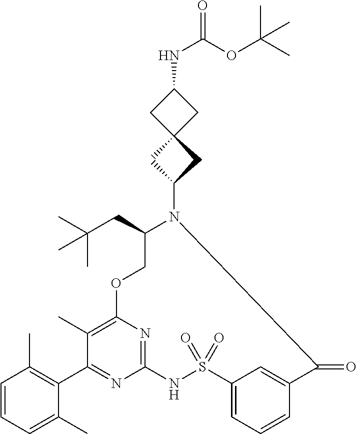 | | | | | +++ | +++ |
| 60 | 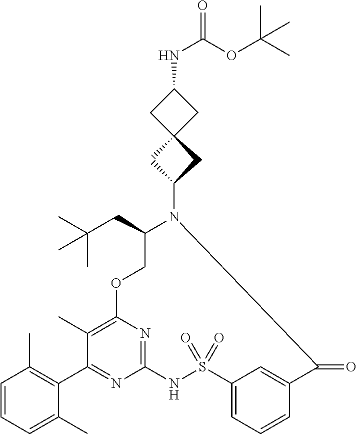 | | | | | +++ | +++ |
| 61 | 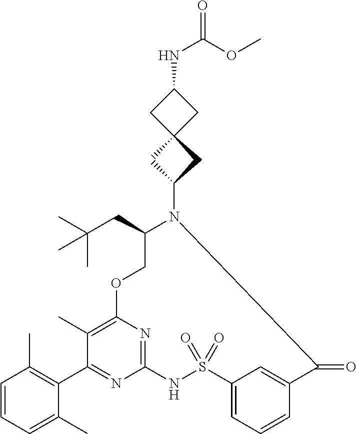 | | | | | +++ | +++ |
| 62 | 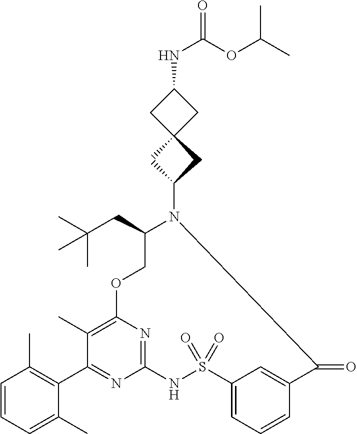 | | | | | +++ | +++ |
| 63 | 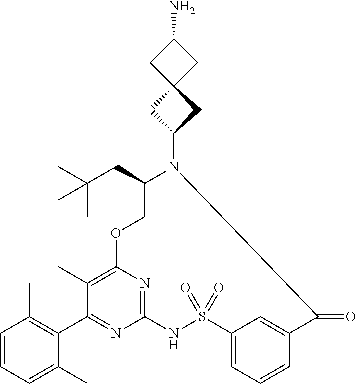 | | | | | +++ | +++ |
| 64 | 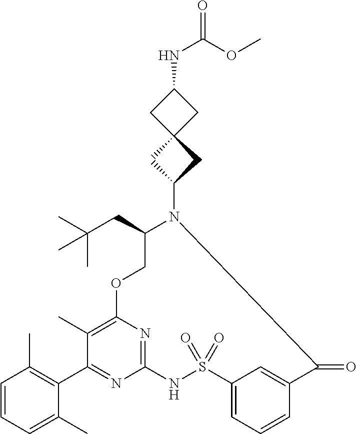 | | | | | +++ | +++ |
| 65 | 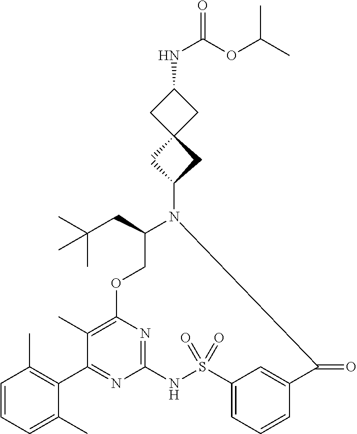 | | | | | +++ | +++ |
| 66 | 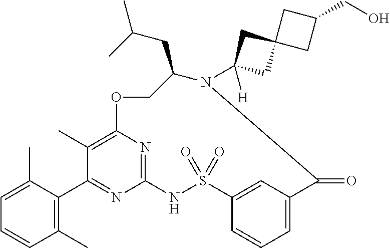 | | | | | +++ | +++ |
| 67 | 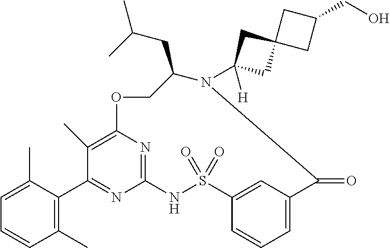 | | | | | +++ | +++ |
| 68 | 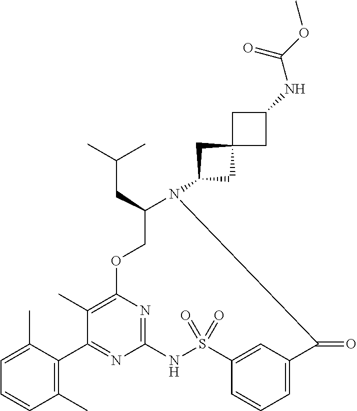 | | | | | +++ | +++ |
| 69 | 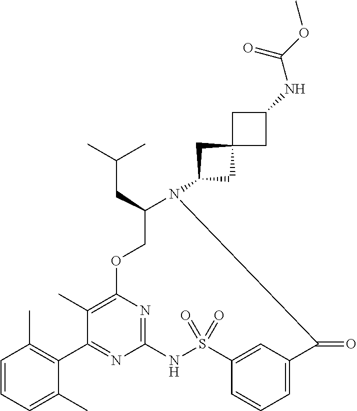 | | | | | +++ | +++ |
| 70 | 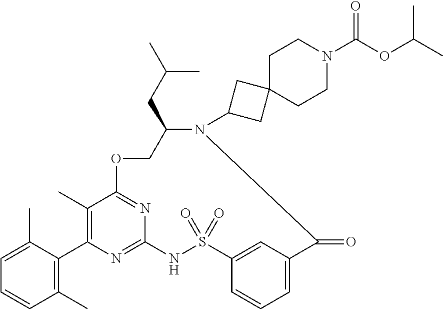 [SMILES: Cc1cccc(C)c1-c1nc2nc(c1C)OC[C@@H](CC(C)C)N(C1CC3(CCN(C(=O)OC(C)C)CC3)C1)C(=O)c1cccc(c1)S(=O)(=O)N2] | | | | | +++ | +++ |
| 71 | 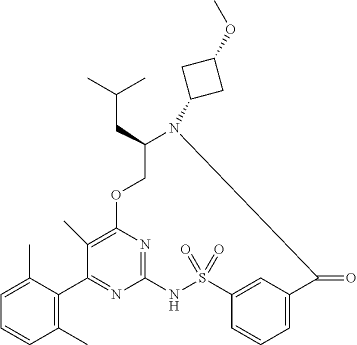 [SMILES: CO[C@H]1C[C@@H](N2C(=O)c3cccc(c3)S(=O)(=O)Nc3nc(c(C)c(-c4c(C)cccc4C)n3)OC[C@H]2CC(C)C)C1] | | | | | +++ | +++ |
| 72 | 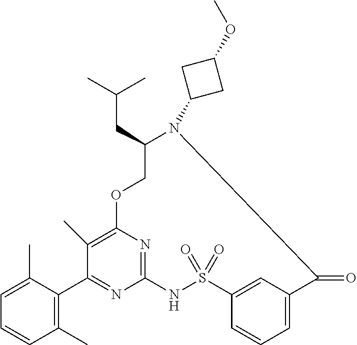 [SMILES: CO[C@H]1C[C@@H](N2C(=O)c3cccc(c3)S(=O)(=O)Nc3nc(c(C)c(-c4c(C)cccc4C)n3)OC[C@H]2CC(C)C)C1] | | | | | +++ | +++ |
| 73 | 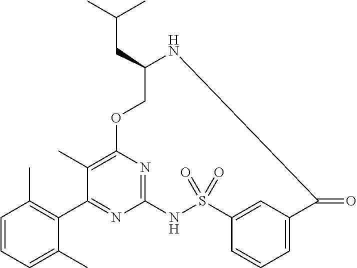 [SMILES: Cc1cccc(C)c1-c1nc2nc(c1C)OC[C@@H](CC(C)C)NC(=O)c1cccc(c1)S(=O)(=O)N2] | | | | | +++ | +++ |
| 74 | 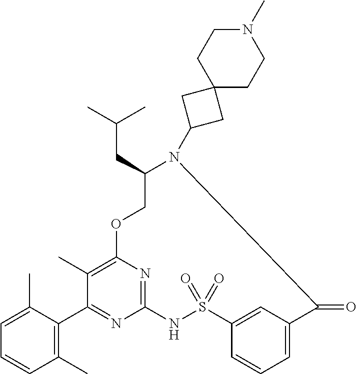 [SMILES: Cc1cccc(C)c1-c1nc2nc(c1C)OC[C@@H](CC(C)C)N(C1CC3(CCN(C)CC3)C1)C(=O)c1cccc(c1)S(=O)(=O)N2] | | | | | +++ | +++ |
| 75 | 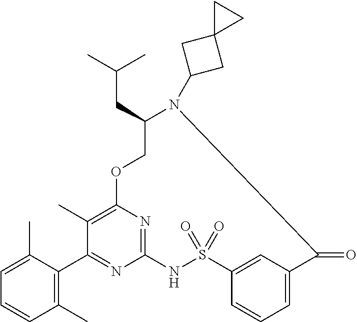 [SMILES: Cc1cccc(C)c1-c1nc2nc(c1C)OC[C@@H](CC(C)C)N(C1CC3(CC3)C1)C(=O)c1cccc(c1)S(=O)(=O)N2] | | | | | +++ | +++ |
| 76 | 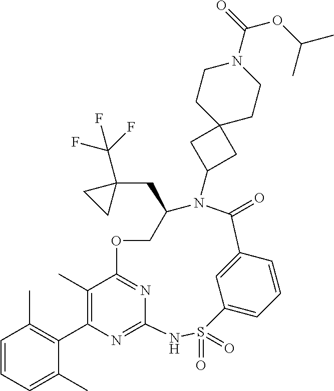 [SMILES: Cc1cccc(C)c1-c1nc2nc(c1C)OC[C@@H](CC1(C(F)(F)F)CC1)N(C1CC3(CCN(C(=O)OC(C)C)CC3)C1)C(=O)c1cccc(c1)S(=O)(=O)N2] | | | | | +++ | +++ |
| 77 | 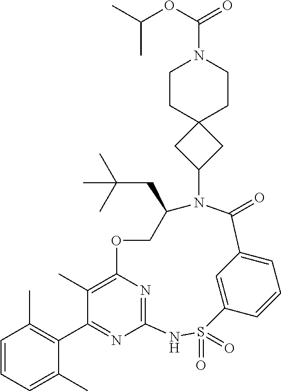 [SMILES: Cc1cccc(C)c1-c1nc2nc(c1C)OC[C@@H](CC(C)(C)C)N(C1CC3(CCN(C(=O)OC(C)C)CC3)C1)C(=O)c1cccc(c1)S(=O)(=O)N2] | | | | | +++ | +++ |
| 78 | 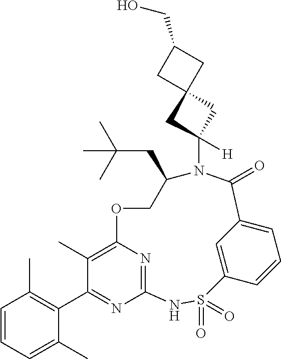 | | | | | +++ | +++ |
| 79 | 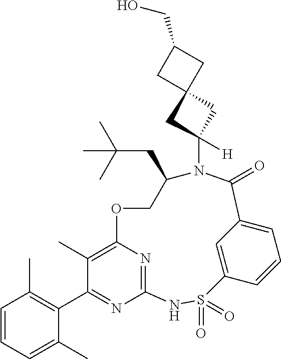 | | | | | +++ | +++ |
| 80 | 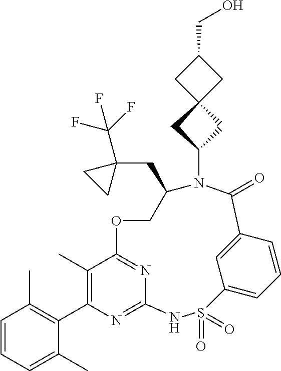 | | | | | +++ | +++ |
| 81 | 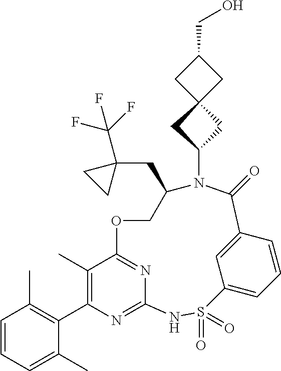 | | | | | +++ | +++ |
| 82 | 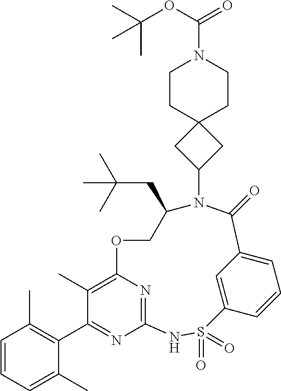 [SMILES: Cc1cccc(C)c1-c1nc2nc(c1C)OC[C@@H](CC(C)(C)C)N(C1CC3(CCN(C(=O)OC(C)(C)C)CC3)C1)C(=O)c1cccc(c1)S(=O)(=O)N2] | | | | | +++ | +++ |
| 83 | 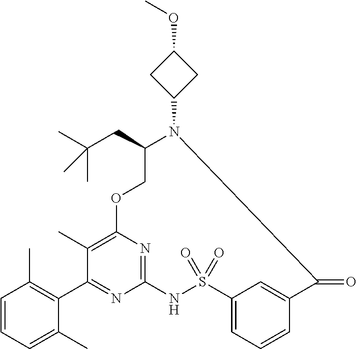 | | | | | +++ | +++ |
| 84 | 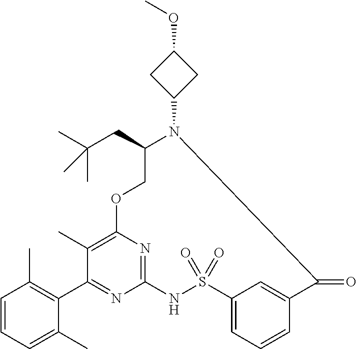 | | | | | +++ | +++ |
| 85 | 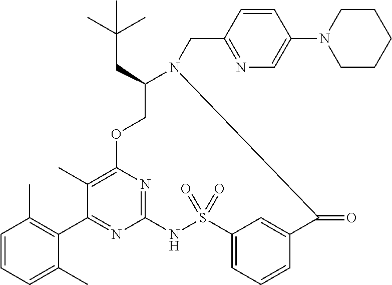 [SMILES: Cc1cccc(C)c1-c1nc2nc(c1C)OC[C@@H](CC(C)(C)C)N(Cc1ccc(N3CCCCC3)cn1)C(=O)c1cccc(c1)S(=O)(=O)N2] | See HBE assay data below | | | | | |
| 86 | 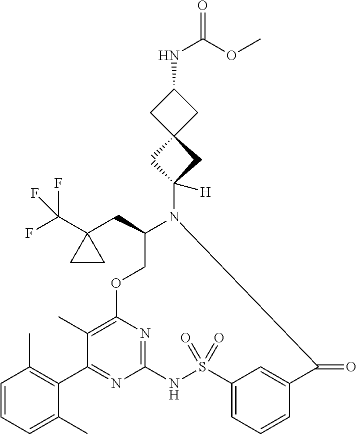 | | | | | +++ | +++ |
| 87 | 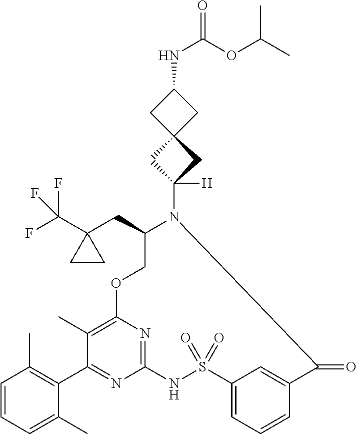 | | | | | +++ | +++ |
| 88 | 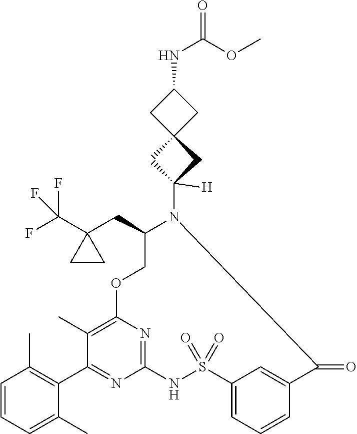 | | | | | +++ | +++ |
| 89 | 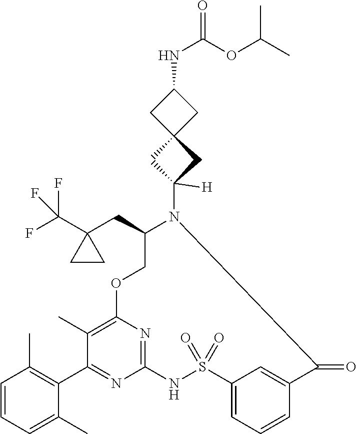 | | | | | +++ | +++ |
| 90 | 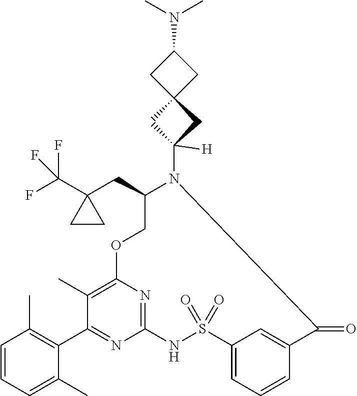 | | | | | +++ | +++ |
| 91 | 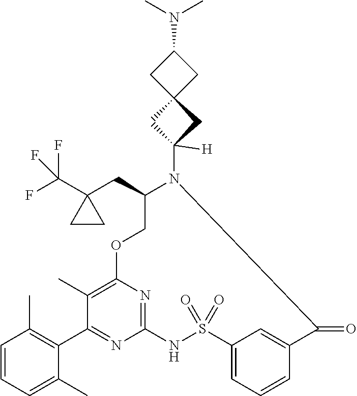 | | | | | +++ | +++ |
| 92 | 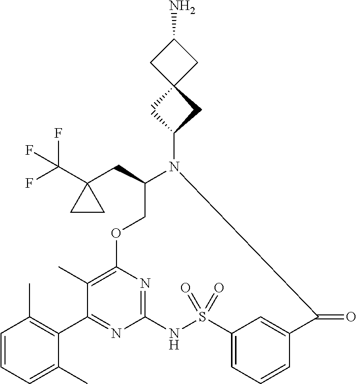 | | | | | +++ | +++ |
| 93 | 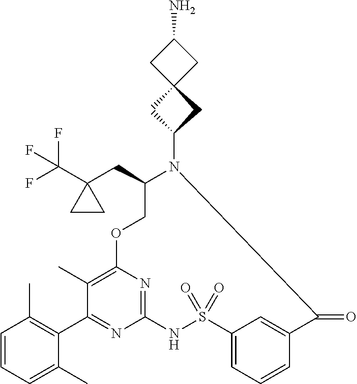 | | | | | +++ | +++ |
| 94 | 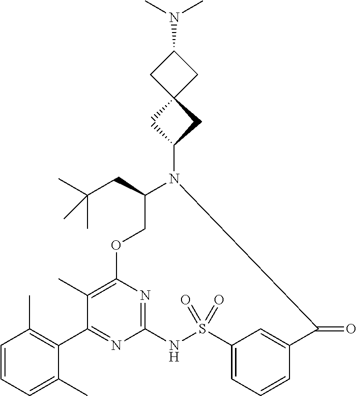 | | | | | +++ | +++ |
| 95 | 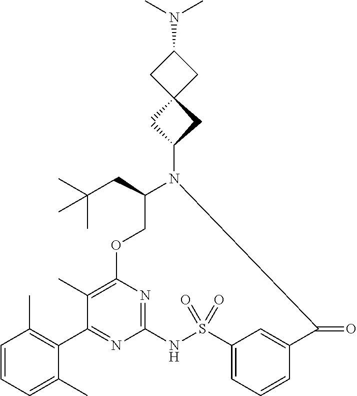 | | | | | +++ | +++ |
| 96 | 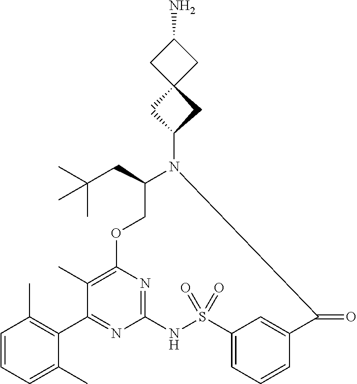 | | | | | +++ | +++ |
| 97 | 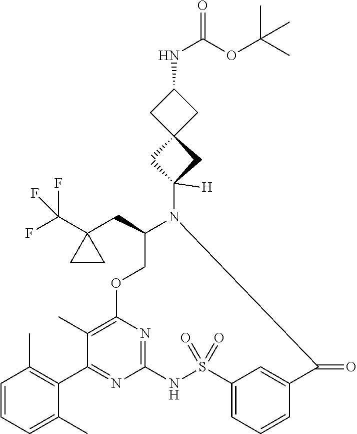 | | | | | +++ | +++ |
| 98 | 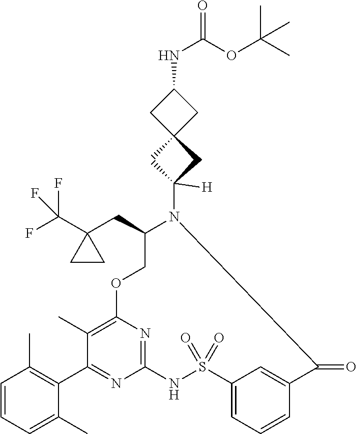 | | | | | +++ | +++ |
| 99 | 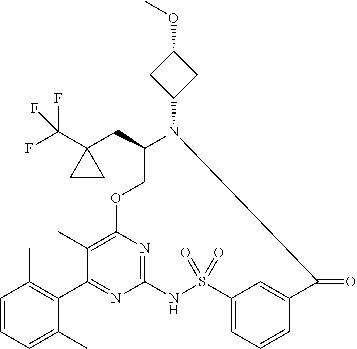 | | | | | +++ | +++ |
| 100 | 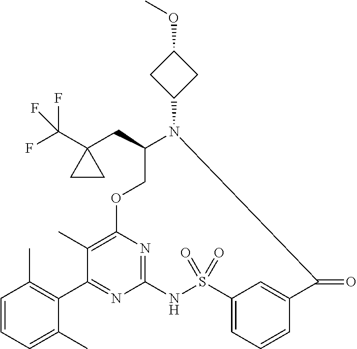 | | | | | +++ | +++ |
| 101 | 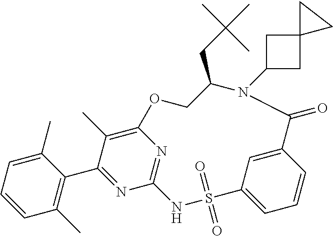 [SMILES: Cc1cccc(C)c1-c1nc2nc(c1C)OC[C@@H](CC(C)(C)C)N(C1CC3(CC3)C1)C(=O)c1cccc(c1)S(=O)(=O)N2] | | | | | +++ | +++ |
| 102 | 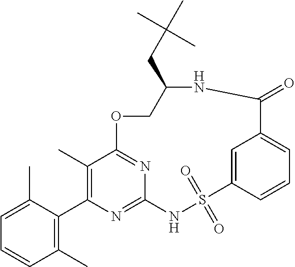 [SMILES: Cc1cccc(C)c1-c1nc2nc(c1C)OC[C@@H](CC(C)(C)C)NC(=O)c1cccc(c1)S(=O)(=O)N2] | | | | | +++ | +++ |
| 103 | 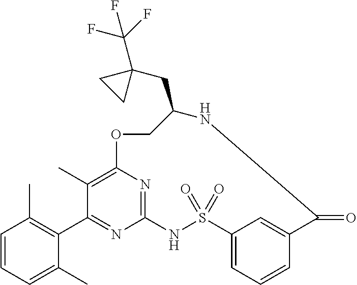 [SMILES: Cc1cccc(C)c1-c1nc2nc(c1C)OC[C@@H](CC1(C(F)(F)F)CC1)NC(=O)c1cccc(c1)S(=O)(=O)N2] | | | | | +++ | +++ |
| 104 | 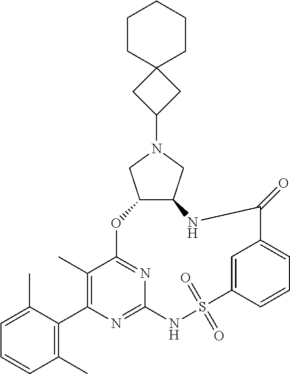 [SMILES: Cc1cccc(C)c1-c1nc2nc(c1C)O[C@@H]1CN(C3CC4(CCCCC4)C3)C[C@H]1NC(=O)c1cccc(c1)S(=O)(=O)N2] | | | | | +++ | +++ |
| 105 | 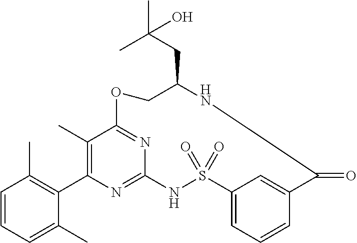 [SMILES: Cc1cccc(C)c1-c1nc2nc(c1C)OC[C@@H](CC(C)(C)O)NC(=O)c1cccc(c1)S(=O)(=O)N2] | | | | | + | +++ |
| 106 | 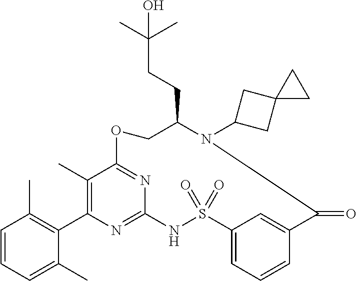 [SMILES: Cc1cccc(C)c1-c1nc2nc(c1C)OC[C@@H](CCC(C)(C)O)N(C1CC3(CC3)C1)C(=O)c1cccc(c1)S(=O)(=O)N2] | | | | | ++ | ++ |
| 107 | 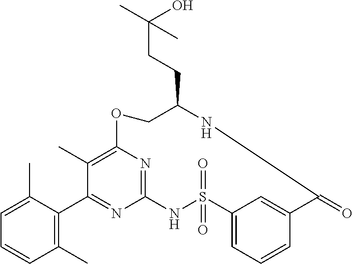 [SMILES: Cc1cccc(C)c1-c1nc2nc(c1C)OC[C@@H](CCC(C)(C)O)NC(=O)c1cccc(c1)S(=O)(=O)N2] | | | | | + | + |
| 108 | 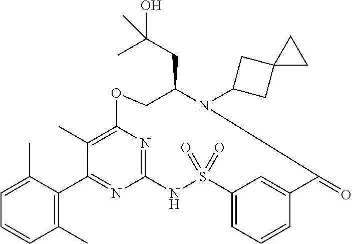 [SMILES: Cc1cccc(C)c1-c1nc2nc(c1C)OC[C@@H](CC(C)(C)O)N(C1CC3(CC3)C1)C(=O)c1cccc(c1)S(=O)(=O)N2] | | | | | ++ | +++ |
| 109 | 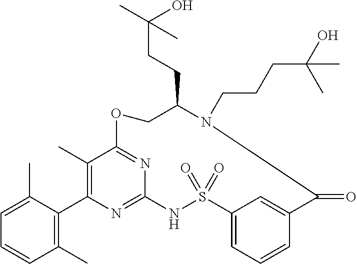 [SMILES: Cc1cccc(C)c1-c1nc2nc(c1C)OC[C@@H](CCC(C)(C)O)N(CCCC(C)(C)O)C(=O)c1cccc(c1)S(=O)(=O)N2] | | | | | + | ++ |
| 110 | 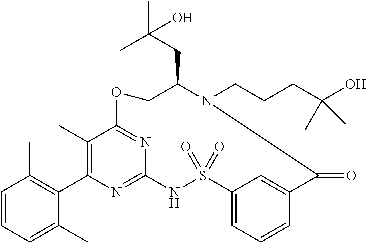 [SMILES: Cc1cccc(C)c1-c1nc2nc(c1C)OC[C@@H](CC(C)(C)O)N(CCCC(C)(C)O)C(=O)c1cccc(c1)S(=O)(=O)N2] | | | | | ND | + |
| 111 | 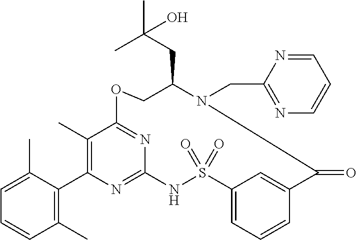 [SMILES: Cc1cccc(C)c1-c1nc2nc(c1C)OC[C@@H](CC(C)(C)O)N(Cc1ncccn1)C(=O)c1cccc(c1)S(=O)(=O)N2] | | | | | ++ | +++ |
| 112 | 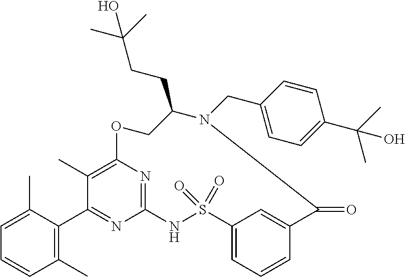 [SMILES: Cc1cccc(C)c1-c1nc2nc(c1C)OC[C@@H](CCC(C)(C)O)N(Cc1ccc(C(C)(C)O)cc1)C(=O)c1cccc(c1)S(=O)(=O)N2] | | | | | ++ | ++ |
| 113 | 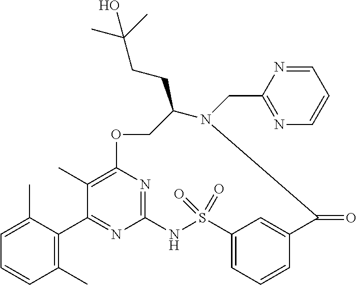 [SMILES: Cc1cccc(C)c1-c1nc2nc(c1C)OC[C@@H](CCC(C)(C)O)N(Cc1ncccn1)C(=O)c1cccc(c1)S(=O)(=O)N2] | | | | | + | ++ |
| 114 | 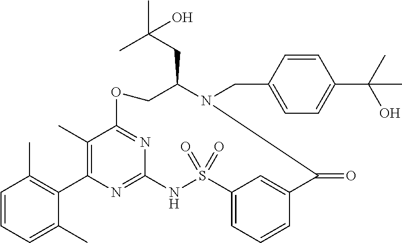 [SMILES: Cc1cccc(C)c1-c1nc2nc(c1C)OC[C@@H](CC(C)(C)O)N(Cc1ccc(C(C)(C)O)cc1)C(=O)c1cccc(c1)S(=O)(=O)N2] | | | | | +++ | +++ |
| 115 | 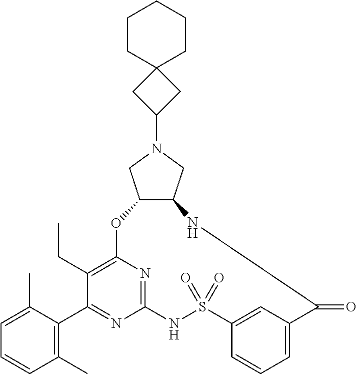 [SMILES: CCc1c2nc(nc1-c1c(C)cccc1C)NS(=O)(=O)c1cccc(c1)C(=O)N[C@@H]1CN(C3CC4(CCCCC4)C3)C[C@H]1O2] | | | | | +++ | +++ |
| 116 | 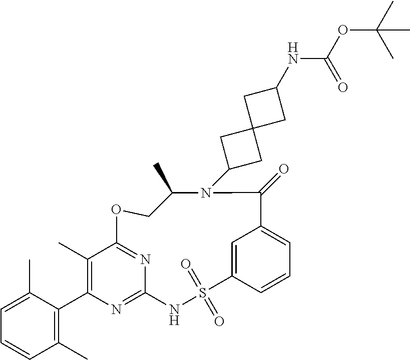 [SMILES: Cc1cccc(C)c1-c1nc2nc(c1C)OC[C@@H](C)N(C1CC3(CC(NC(=O)OC(C)(C)C)C3)C1)C(=O)c1cccc(c1)S(=O)(=O)N2] | | | | | +++ | ++ |
| 117 | 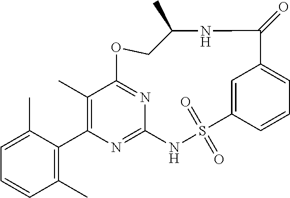 [SMILES: Cc1cccc(C)c1-c1nc2nc(c1C)OC[C@@H](C)NC(=O)c1cccc(c1)S(=O)(=O)N2] | | | | | ++ | ++ |
| 118 | 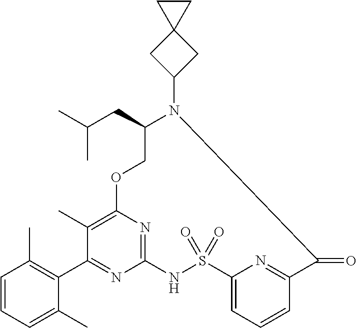 [SMILES: Cc1cccc(C)c1-c1nc2nc(c1C)OC[C@@H](CC(C)C)N(C1CC3(CC3)C1)C(=O)c1cccc(n1)S(=O)(=O)N2] | | | | | +++ | +++ |
| 119 | 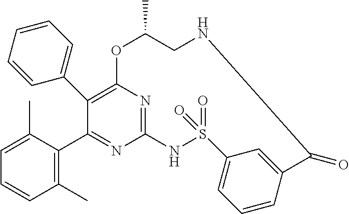 [SMILES: Cc1cccc(C)c1-c1nc2nc(c1-c1ccccc1)O[C@H](C)CNC(=O)c1cccc(c1)S(=O)(=O)N2] | | | | | +++ | +++ |
| 120 | 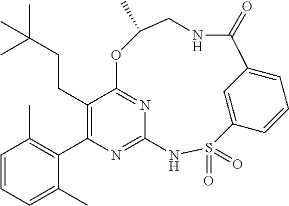 [SMILES: Cc1cccc(C)c1-c1nc2nc(c1CCC(C)(C)C)O[C@H](C)CNC(=O)c1cccc(c1)S(=O)(=O)N2] | | | | | +++ | +++ |
| 121 | 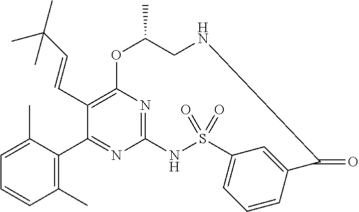 [SMILES: Cc1cccc(C)c1-c1nc2nc(c1/C=C/C(C)(C)C)O[C@H](C)CNC(=O)c1cccc(c1)S(=O)(=O)N2] | | | | | +++ | +++ |
| 122 | 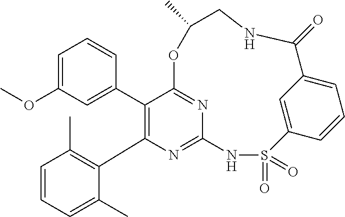 [SMILES: COc1cccc(-c2c3nc(nc2-c2c(C)cccc2C)NS(=O)(=O)c2cccc(c2)C(=O)NC[C@@H](C)O3)c1] | | | | | +++ | +++ |
| 123 | 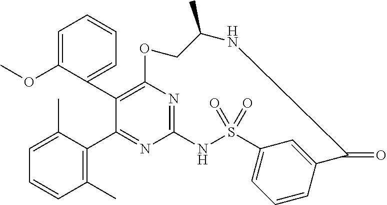 [SMILES: COc1ccccc1-c1c2nc(nc1-c1c(C)cccc1C)NS(=O)(=O)c1cccc(c1)C(=O)N[C@H](C)CO2] | | | | | ++ | +++ |
| 124 | 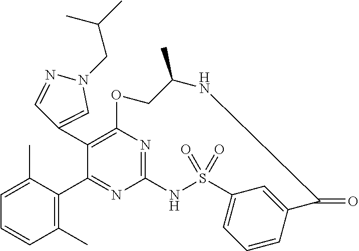 [SMILES: Cc1cccc(C)c1-c1nc2nc(c1-c1cnn(CC(C)C)c1)OC[C@@H](C)NC(=O)c1cccc(c1)S(=O)(=O)N2] | | | | | + | ++ |
| 125 | 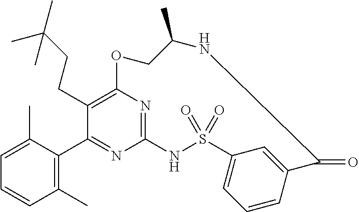 [SMILES: Cc1cccc(C)c1-c1nc2nc(c1CCC(C)(C)C)OC[C@@H](C)NC(=O)c1cccc(c1)S(=O)(=O)N2] | | | | | +++ | ++ |
| 126 | 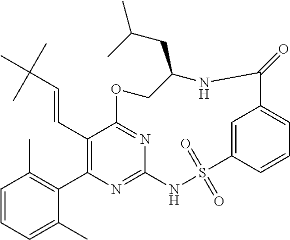 [SMILES: Cc1cccc(C)c1-c1nc2nc(c1/C=C/C(C)(C)C)OC[C@@H](CC(C)C)NC(=O)c1cccc(c1)S(=O)(=O)N2] | | | | | +++ | +++ |
| 127 | 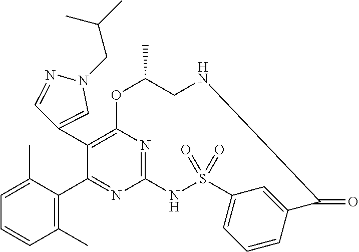 [SMILES: Cc1cccc(C)c1-c1nc2nc(c1-c1cnn(CC(C)C)c1)O[C@H](C)CNC(=O)c1cccc(c1)S(=O)(=O)N2] | | | | | ++ | +++ |
| 128 | 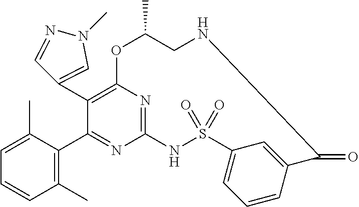 [SMILES: Cc1cccc(C)c1-c1nc2nc(c1-c1cnn(C)c1)O[C@H](C)CNC(=O)c1cccc(c1)S(=O)(=O)N2] | | | | | ND | + |
| 129 | 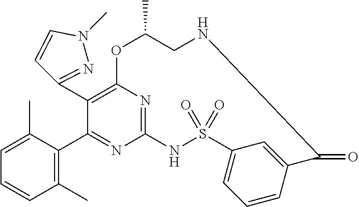 [SMILES: Cc1cccc(C)c1-c1nc2nc(c1-c1ccn(C)n1)O[C@H](C)CNC(=O)c1cccc(c1)S(=O)(=O)N2] | | | | | ND | + |
| 130 | 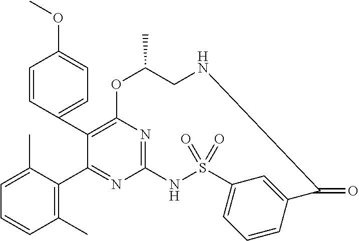 [SMILES: COc1ccc(-c2c3nc(nc2-c2c(C)cccc2C)NS(=O)(=O)c2cccc(c2)C(=O)NC[C@@H](C)O3)cc1] | | | | | +++ | +++ |
| 131 | 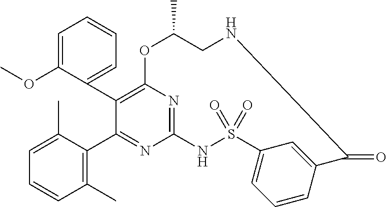 [SMILES: COc1ccccc1-c1c2nc(nc1-c1c(C)cccc1C)NS(=O)(=O)c1cccc(c1)C(=O)NC[C@@H](C)O2] | | | | | ++ | +++ |
| 132 | 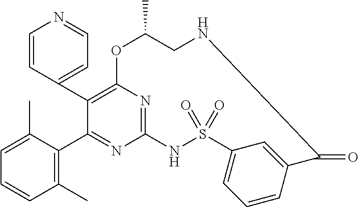 [SMILES: Cc1cccc(C)c1-c1nc2nc(c1-c1ccncc1)O[C@H](C)CNC(=O)c1cccc(c1)S(=O)(=O)N2] | | | | | ND | + |
| 133 | 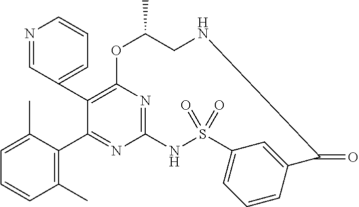 [SMILES: Cc1cccc(C)c1-c1nc2nc(c1-c1cccnc1)O[C@H](C)CNC(=O)c1cccc(c1)S(=O)(=O)N2] | | | | | ND | + |
| 134 | 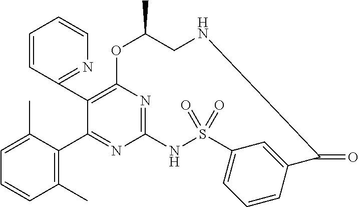 [SMILES: Cc1cccc(C)c1-c1nc2nc(c1-c1ccccn1)O[C@@H](C)CNC(=O)c1cccc(c1)S(=O)(=O)N2] | | | | | + | + |
| 135 | 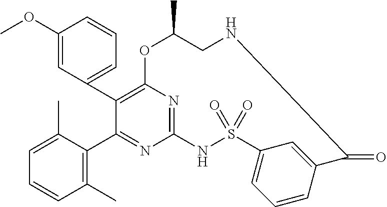 [SMILES: COc1cccc(-c2c3nc(nc2-c2c(C)cccc2C)NS(=O)(=O)c2cccc(c2)C(=O)NC[C@H](C)O3)c1] | | | | | +++ | ++ |
| 136 | 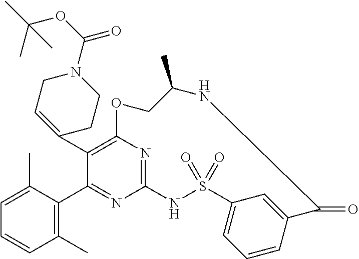 [SMILES: Cc1cccc(C)c1-c1nc2nc(c1C1=CCN(C(=O)OC(C)(C)C)CC1)OC[C@@H](C)NC(=O)c1cccc(c1)S(=O)(=O)N2] | | | | | ++ | ++ |
| 137 | 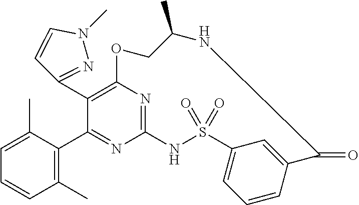 [SMILES: Cc1cccc(C)c1-c1nc2nc(c1-c1ccn(C)n1)OC[C@@H](C)NC(=O)c1cccc(c1)S(=O)(=O)N2] | | | | | ND | + |
| 138 | 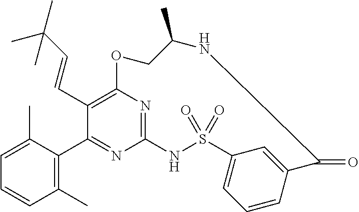 [SMILES: Cc1cccc(C)c1-c1nc2nc(c1/C=C/C(C)(C)C)OC[C@@H](C)NC(=O)c1cccc(c1)S(=O)(=O)N2] | | | | | +++ | ++ |
| 139 | 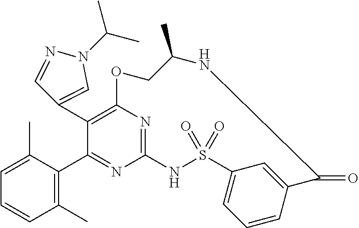 [SMILES: Cc1cccc(C)c1-c1nc2nc(c1-c1cnn(C(C)C)c1)OC[C@@H](C)NC(=O)c1cccc(c1)S(=O)(=O)N2] | | | | | + | + |
| 140 | 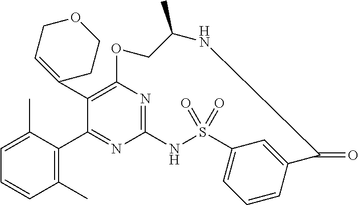 [SMILES: Cc1cccc(C)c1-c1nc2nc(c1C1=CCOCC1)OC[C@@H](C)NC(=O)c1cccc(c1)S(=O)(=O)N2] | | | | | + | ++ |
| 141 | 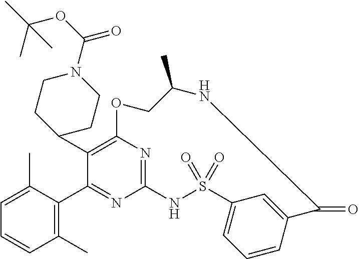 [SMILES: Cc1cccc(C)c1-c1nc2nc(c1C1CCN(C(=O)OC(C)(C)C)CC1)OC[C@@H](C)NC(=O)c1cccc(c1)S(=O)(=O)N2] | | | | | ND | + |
| 142 | 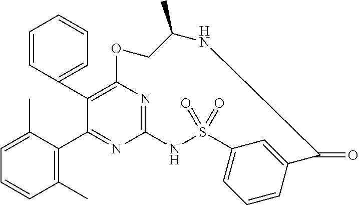 [SMILES: Cc1cccc(C)c1-c1nc2nc(c1-c1ccccc1)OC[C@@H](C)NC(=O)c1cccc(c1)S(=O)(=O)N2] | | | | | +++ | ++ |
| 143 | 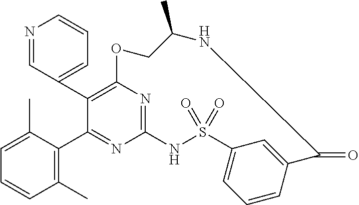 [SMILES: Cc1cccc(C)c1-c1nc2nc(c1-c1cccnc1)OC[C@@H](C)NC(=O)c1cccc(c1)S(=O)(=O)N2] | | | | | ND | + |
| 144 | 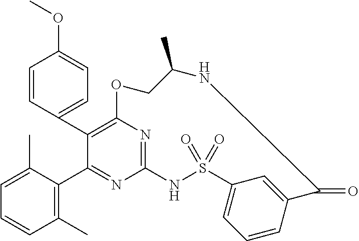 [SMILES: COc1ccc(-c2c3nc(nc2-c2c(C)cccc2C)NS(=O)(=O)c2cccc(c2)C(=O)N[C@H](C)CO3)cc1] | | | | | +++ | +++ |
| 145 | 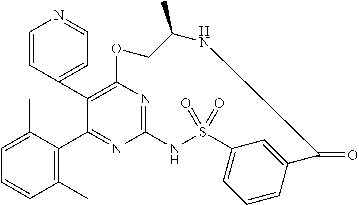 [SMILES: Cc1cccc(C)c1-c1nc2nc(c1-c1ccncc1)OC[C@@H](C)NC(=O)c1cccc(c1)S(=O)(=O)N2] | | | | | ND | + |
| 146 | 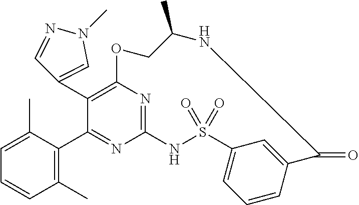 [SMILES: Cc1cccc(C)c1-c1nc2nc(c1-c1cnn(C)c1)OC[C@@H](C)NC(=O)c1cccc(c1)S(=O)(=O)N2] | | | | | ND | + |
| 147 | 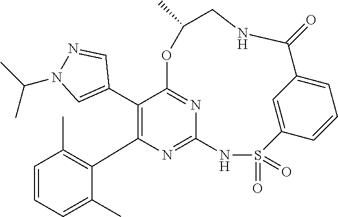 [SMILES: Cc1cccc(C)c1-c1nc2nc(c1-c1cnn(C(C)C)c1)O[C@H](C)CNC(=O)c1cccc(c1)S(=O)(=O)N2] | | | | | + | ++ |
| 148 | 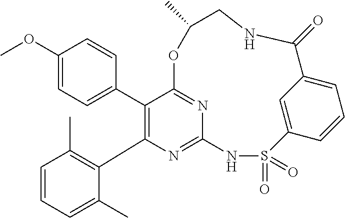 [SMILES: COc1ccc(-c2c3nc(nc2-c2c(C)cccc2C)NS(=O)(=O)c2cccc(c2)C(=O)NC[C@@H](C)O3)cc1] | | | | | +++ | +++ |
| 149 | 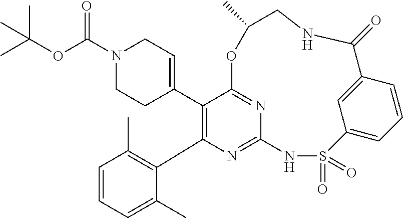 [SMILES: Cc1cccc(C)c1-c1nc2nc(c1C1=CCN(C(=O)OC(C)(C)C)CC1)O[C@H](C)CNC(=O)c1cccc(c1)S(=O)(=O)N2] | | | | | +++ | ++ |
| 150 | 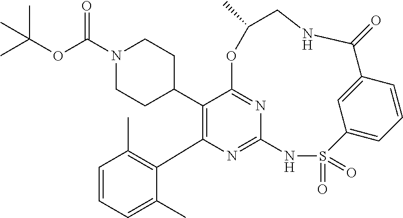 [SMILES: Cc1cccc(C)c1-c1nc2nc(c1C1CCN(C(=O)OC(C)(C)C)CC1)O[C@H](C)CNC(=O)c1cccc(c1)S(=O)(=O)N2] | | | | | ++ | ++ |
| 151 | 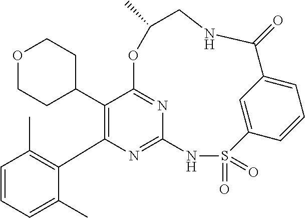 [SMILES: Cc1cccc(C)c1-c1nc2nc(c1C1CCOCC1)O[C@H](C)CNC(=O)c1cccc(c1)S(=O)(=O)N2] | | | | | ND | ++ |
| 152 | 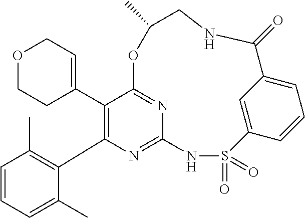 [SMILES: Cc1cccc(C)c1-c1nc2nc(c1C1=CCOCC1)O[C@H](C)CNC(=O)c1cccc(c1)S(=O)(=O)N2] | | | | | + | ++ |
| 153 | 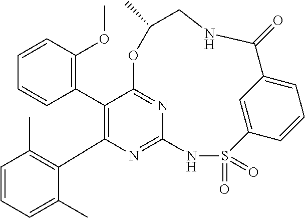 [SMILES: COc1ccccc1-c1c2nc(nc1-c1c(C)cccc1C)NS(=O)(=O)c1cccc(c1)C(=O)NC[C@@H](C)O2] | | | | | +++ | +++ |
| 154 | 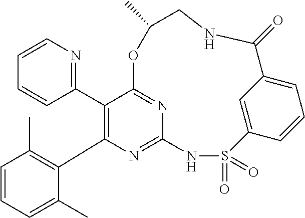 [SMILES: Cc1cccc(C)c1-c1nc2nc(c1-c1ccccn1)O[C@H](C)CNC(=O)c1cccc(c1)S(=O)(=O)N2] | | | | | + | + |
| 155 | 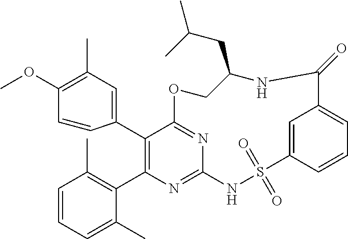 [SMILES: COc1ccc(-c2c3nc(nc2-c2c(C)cccc2C)NS(=O)(=O)c2cccc(c2)C(=O)N[C@H](CC(C)C)CO3)cc1C] | | | | | +++ | +++ |
| 156 | 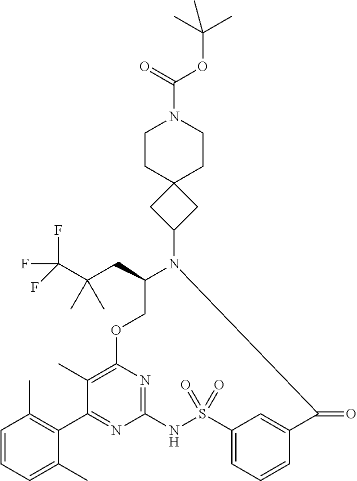 [SMILES: Cc1cccc(C)c1-c1nc2nc(c1C)OC[C@@H](CC(C)(C)C(F)(F)F)N(C1CC3(CCN(C(=O)OC(C)(C)C)CC3)C1)C(=O)c1cccc(c1)S(=O)(=O)N2] | | | | | +++ | +++ |
| 157 | 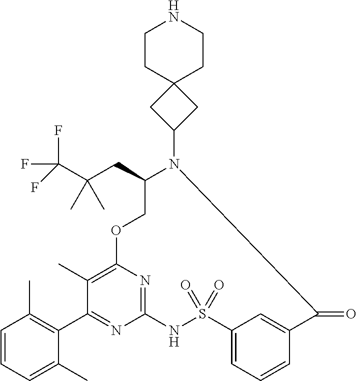 [SMILES: Cc1cccc(C)c1-c1nc2nc(c1C)OC[C@@H](CC(C)(C)C(F)(F)F)N(C1CC3(CCNCC3)C1)C(=O)c1cccc(c1)S(=O)(=O)N2] | | | | | +++ | +++ |
| 158 | 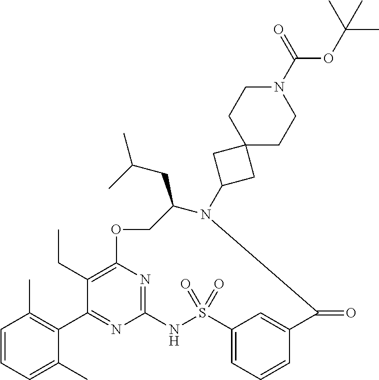 [SMILES: CCc1c2nc(nc1-c1c(C)cccc1C)NS(=O)(=O)c1cccc(c1)C(=O)N(C1CC3(CCN(C(=O)OC(C)(C)C)CC3)C1)[C@H](CC(C)C)CO2] | | | +++ | +++ | +++ | +++ |
| 159 | 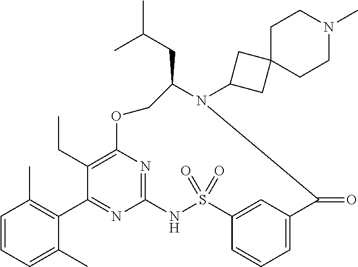 [SMILES: CCc1c2nc(nc1-c1c(C)cccc1C)NS(=O)(=O)c1cccc(c1)C(=O)N(C1CC3(CCN(C)CC3)C1)[C@H](CC(C)C)CO2] | | | +++ | +++ | +++ | +++ |
| 160 | 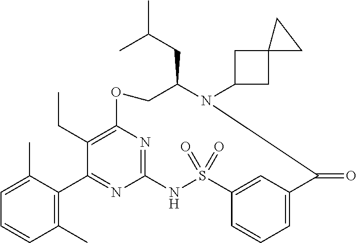 [SMILES: CCc1c2nc(nc1-c1c(C)cccc1C)NS(=O)(=O)c1cccc(c1)C(=O)N(C1CC3(CC3)C1)[C@H](CC(C)C)CO2] | | | | | +++ | +++ |
| 161 | 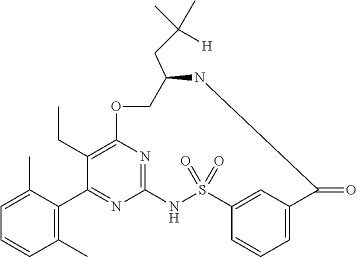 [SMILES: CCc1c2nc(nc1-c1c(C)cccc1C)NS(=O)(=O)c1cccc(c1)C(=O)N[C@H](CC(C)C)CO2] | | | | | +++ | +++ |
| 162 | 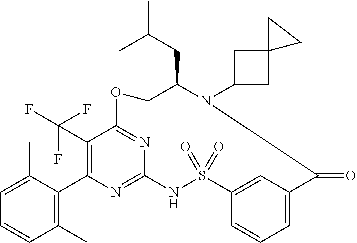 [SMILES: Cc1cccc(C)c1-c1nc2nc(c1C(F)(F)F)OC[C@@H](CC(C)C)N(C1CC3(CC3)C1)C(=O)c1cccc(c1)S(=O)(=O)N2] | | | | | +++ | +++ |
| 163 | 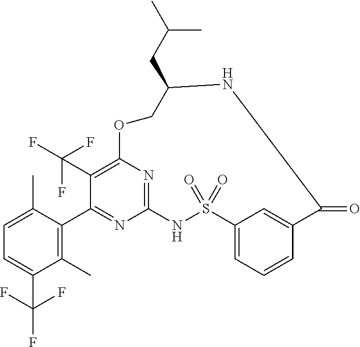 [SMILES: Cc1ccc(C(F)(F)F)c(C)c1-c1nc2nc(c1C(F)(F)F)OC[C@@H](CC(C)C)NC(=O)c1cccc(c1)S(=O)(=O)N2] | | | | | +++ | +++ |
| 164 | 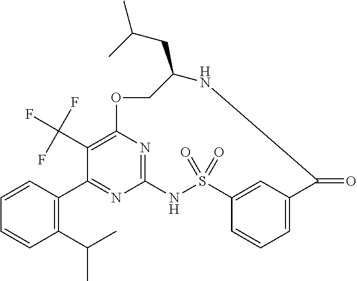 [SMILES: CC(C)C[C@@H]1COc2nc(nc(-c3ccccc3C(C)C)c2C(F)(F)F)NS(=O)(=O)c2cccc(c2)C(=O)N1] | | | +++ | +++ | +++ | +++ |
| 165 | 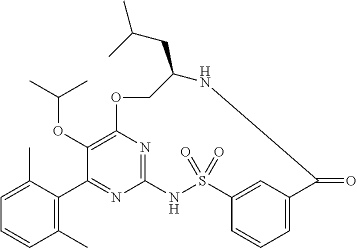 [SMILES: Cc1cccc(C)c1-c1nc2nc(c1OC(C)C)OC[C@@H](CC(C)C)NC(=O)c1cccc(c1)S(=O)(=O)N2] | | | +++ | +++ | +++ | +++ |
| 166 | 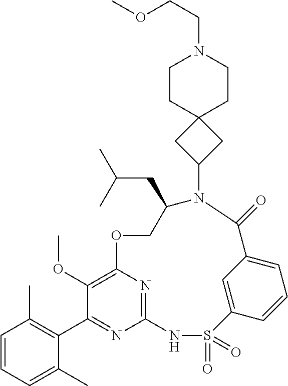 [SMILES: COCCN1CCC2(CC1)CC(N1C(=O)c3cccc(c3)S(=O)(=O)Nc3nc(c(OC)c(-c4c(C)cccc4C)n3)OC[C@H]1CC(C)C)C2] | | | | | +++ | +++ |
| 167 | 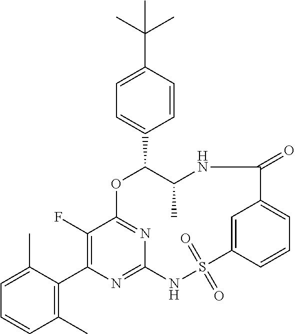 [SMILES: Cc1cccc(C)c1-c1nc2nc(c1F)O[C@H](c1ccc(C(C)(C)C)cc1)[C@@H](C)NC(=O)c1cccc(c1)S(=O)(=O)N2] | | | | | +++ | +++ |
| 168 | 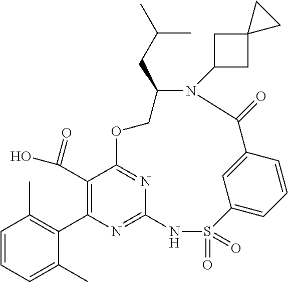 [SMILES: Cc1cccc(C)c1-c1nc2nc(c1C(=O)O)OC[C@@H](CC(C)C)N(C1CC3(CC3)C1)C(=O)c1cccc(c1)S(=O)(=O)N2] | | | | | ND | + |
| 169 | 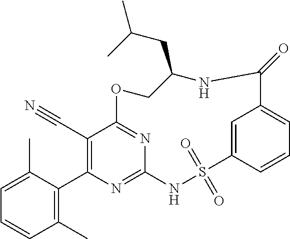 [SMILES: Cc1cccc(C)c1-c1nc2nc(c1C#N)OC[C@@H](CC(C)C)NC(=O)c1cccc(c1)S(=O)(=O)N2] | | | | | + | +++ |
| 170 | 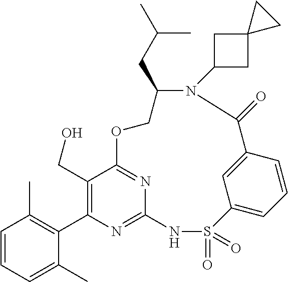 [SMILES: Cc1cccc(C)c1-c1nc2nc(c1CO)OC[C@@H](CC(C)C)N(C1CC3(CC3)C1)C(=O)c1cccc(c1)S(=O)(=O)N2] | | | | | +++ | ++ |
| 171 | 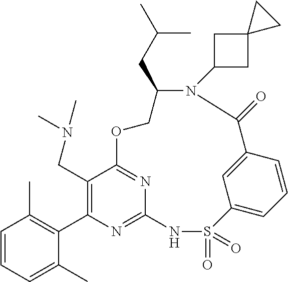 [SMILES: Cc1cccc(C)c1-c1nc2nc(c1CN(C)C)OC[C@@H](CC(C)C)N(C1CC3(CC3)C1)C(=O)c1cccc(c1)S(=O)(=O)N2] | | | | | +++ | ++ |
| 172 | 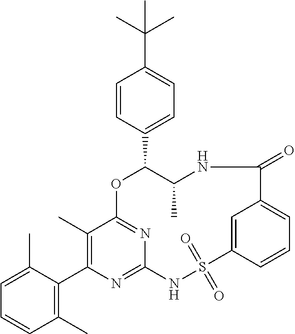 [SMILES: Cc1cccc(C)c1-c1nc2nc(c1C)O[C@H](c1ccc(C(C)(C)C)cc1)[C@@H](C)NC(=O)c1cccc(c1)S(=O)(=O)N2] | | | | | +++ | +++ |
| 173 | 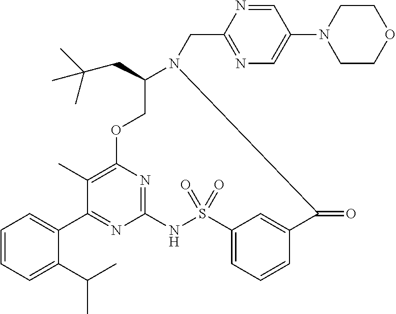 [SMILES: Cc1c2nc(nc1-c1ccccc1C(C)C)NS(=O)(=O)c1cccc(c1)C(=O)N(Cc1ncc(N3CCOCC3)cn1)[C@H](CC(C)(C)C)CO2] | See HBE assay data below | | | | | |
| 174 | 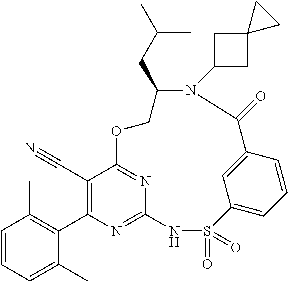 [SMILES: Cc1cccc(C)c1-c1nc2nc(c1C#N)OC[C@@H](CC(C)C)N(C1CC3(CC3)C1)C(=O)c1cccc(c1)S(=O)(=O)N2] | | | | | +++ | ++ |
| 175 | 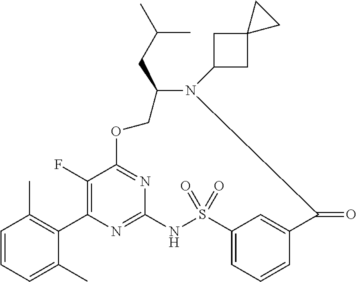 [SMILES: Cc1cccc(C)c1-c1nc2nc(c1F)OC[C@@H](CC(C)C)N(C1CC3(CC3)C1)C(=O)c1cccc(c1)S(=O)(=O)N2] | | | | | +++ | +++ |
| 176 | 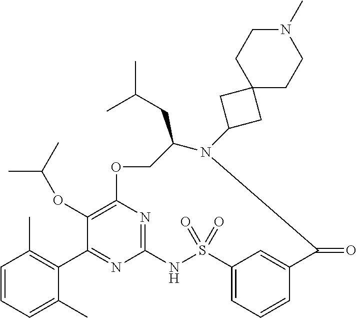 [SMILES: Cc1cccc(C)c1-c1nc2nc(c1OC(C)C)OC[C@@H](CC(C)C)N(C1CC3(CCN(C)CC3)C1)C(=O)c1cccc(c1)S(=O)(=O)N2] | | | +++ | +++ | +++ | +++ |
| 177 | 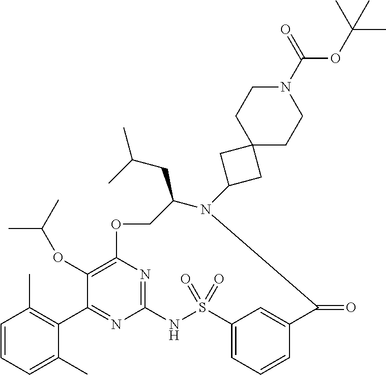 [SMILES: Cc1cccc(C)c1-c1nc2nc(c1OC(C)C)OC[C@@H](CC(C)C)N(C1CC3(CCN(C(=O)OC(C)(C)C)CC3)C1)C(=O)c1cccc(c1)S(=O)(=O)N2] | | | +++ | +++ | +++ | +++ |
| 178 | 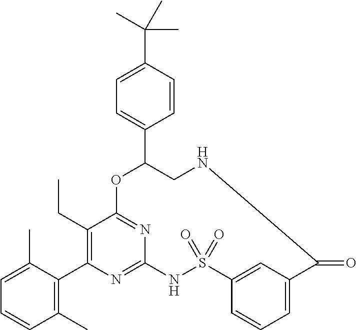 [SMILES: CCc1c2nc(nc1-c1c(C)cccc1C)NS(=O)(=O)c1cccc(c1)C(=O)NCC(c1ccc(C(C)(C)C)cc1)O2] | | | +++ | +++ | | |
| 179 | 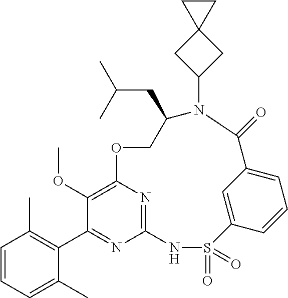 [SMILES: COc1c2nc(nc1-c1c(C)cccc1C)NS(=O)(=O)c1cccc(c1)C(=O)N(C1CC3(CC3)C1)[C@H](CC(C)C)CO2] | | | | | +++ | +++ |
| 180 | 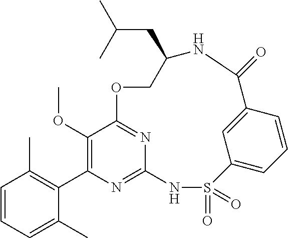 [SMILES: COc1c2nc(nc1-c1c(C)cccc1C)NS(=O)(=O)c1cccc(c1)C(=O)N[C@H](CC(C)C)CO2] | | | | | +++ | +++ |
| 181 | 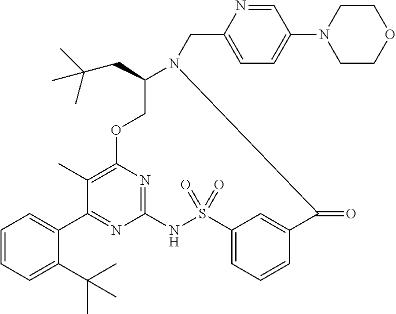 [SMILES: Cc1c2nc(nc1-c1ccccc1C(C)(C)C)NS(=O)(=O)c1cccc(c1)C(=O)N(Cc1ccc(N3CCOCC3)cn1)[C@H](CC(C)(C)C)CO2] | See HBE assay data below | | | | | |
| 182 | 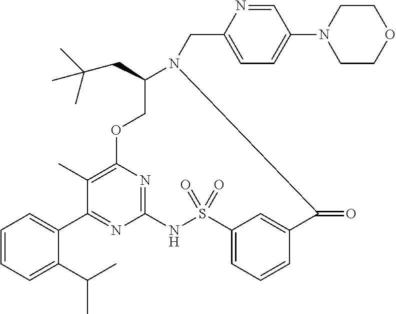 [SMILES: Cc1c2nc(nc1-c1ccccc1C(C)C)NS(=O)(=O)c1cccc(c1)C(=O)N(Cc1ccc(N3CCOCC3)cn1)[C@H](CC(C)(C)C)CO2] | See HBE assay data below | | | | | |
| 183 | 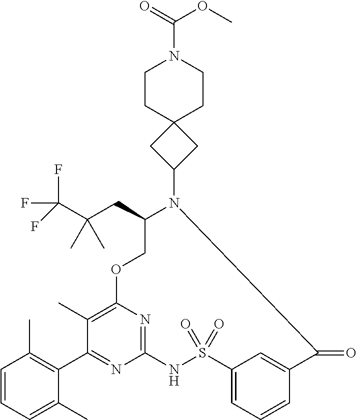 [SMILES: COC(=O)N1CCC2(CC1)CC(N1C(=O)c3cccc(c3)S(=O)(=O)Nc3nc(c(C)c(-c4c(C)cccc4C)n3)OC[C@H]1CC(C)(C)C(F)(F)F)C2] | | | | | +++ | +++ |
| 184 | 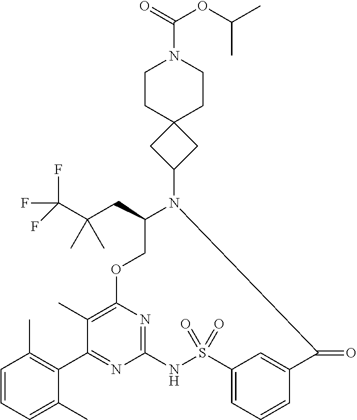 [SMILES: Cc1cccc(C)c1-c1nc2nc(c1C)OC[C@@H](CC(C)(C)C(F)(F)F)N(C1CC3(CCN(C(=O)OC(C)C)CC3)C1)C(=O)c1cccc(c1)S(=O)(=O)N2] | | | | | +++ | +++ |
| 185 | 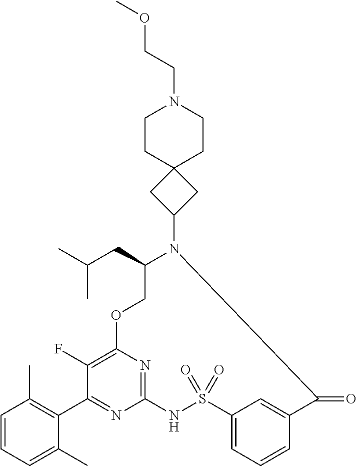 [SMILES: COCCN1CCC2(CC1)CC(N1C(=O)c3cccc(c3)S(=O)(=O)Nc3nc(c(F)c(-c4c(C)cccc4C)n3)OC[C@H]1CC(C)C)C2] | | | | | +++ | +++ |
| 186 | 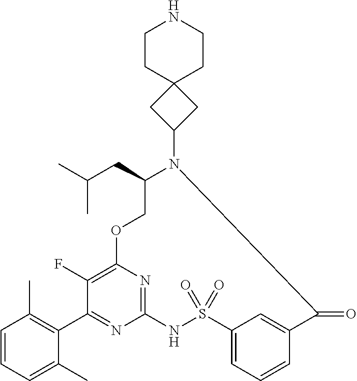 [SMILES: Cc1cccc(C)c1-c1nc2nc(c1F)OC[C@@H](CC(C)C)N(C1CC3(CCNCC3)C1)C(=O)c1cccc(c1)S(=O)(=O)N2] | | | | | +++ | ++ |
| 187 | 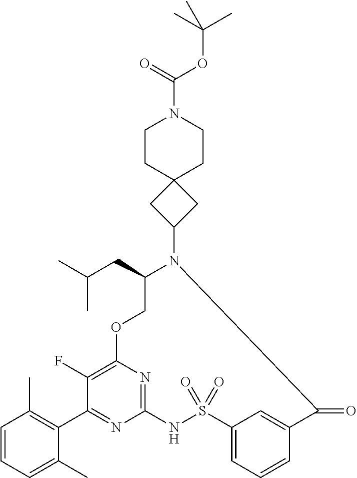 [SMILES: Cc1cccc(C)c1-c1nc2nc(c1F)OC[C@@H](CC(C)C)N(C1CC3(CCN(C(=O)OC(C)(C)C)CC3)C1)C(=O)c1cccc(c1)S(=O)(=O)N2] | | | | | +++ | +++ |
| 188 | 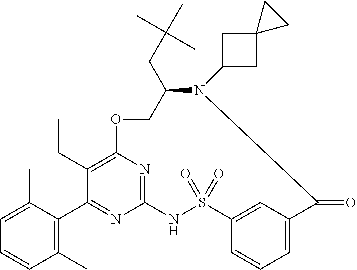 [SMILES: CCc1c2nc(nc1-c1c(C)cccc1C)NS(=O)(=O)c1cccc(c1)C(=O)N(C1CC3(CC3)C1)[C@H](CC(C)(C)C)CO2] | | | | | +++ | +++ |
| 189 | 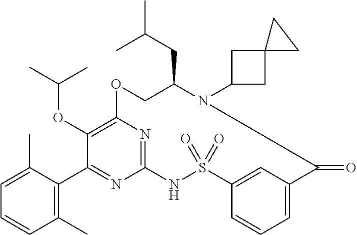 [SMILES: Cc1cccc(C)c1-c1nc2nc(c1OC(C)C)OC[C@@H](CC(C)C)N(C1CC3(CC3)C1)C(=O)c1cccc(c1)S(=O)(=O)N2] | | | | | +++ | +++ |
| 190 | 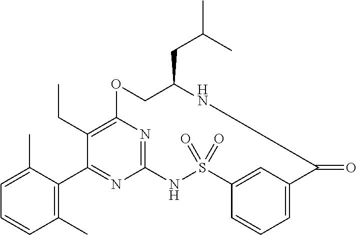 [SMILES: CCc1c2nc(nc1-c1c(C)cccc1C)NS(=O)(=O)c1cccc(c1)C(=O)N[C@H](CC(C)C)CO2] | | | +++ | +++ | +++ | +++ |
| 201 | 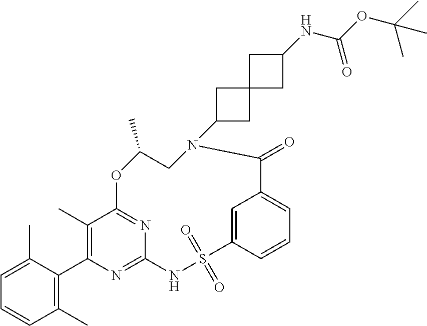 [SMILES: Cc1cccc(C)c1-c1nc2nc(c1C)O[C@H](C)CN(C1CC3(CC(NC(=O)OC(C)(C)C)C3)C1)C(=O)c1cccc(c1)S(=O)(=O)N2] | | | | | +++ | ++ |
| 202 | 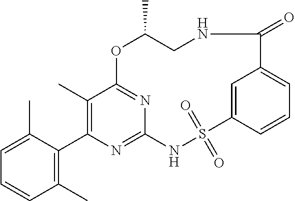 [SMILES: Cc1cccc(C)c1-c1nc2nc(c1C)O[C@H](C)CNC(=O)c1cccc(c1)S(=O)(=O)N2] | | | | | ++ | ++ |
| 203 | 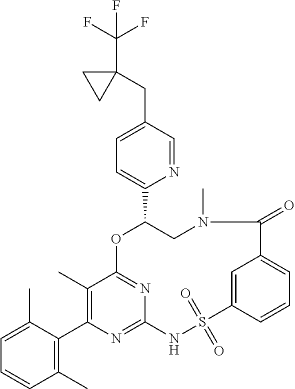 [SMILES: Cc1cccc(C)c1-c1nc2nc(c1C)O[C@H](c1ccc(CC3(C(F)(F)F)CC3)cn1)CN(C)C(=O)c1cccc(c1)S(=O)(=O)N2] | | | | | +++ | +++ |
| 204 | 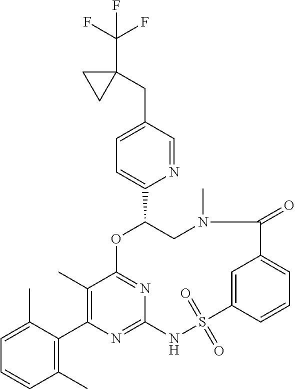 [SMILES: Cc1cccc(C)c1-c1nc2nc(c1C)O[C@H](c1ccc(CC3(C(F)(F)F)CC3)cn1)CN(C)C(=O)c1cccc(c1)S(=O)(=O)N2] | | | | | +++ | ++ |
| 205 | 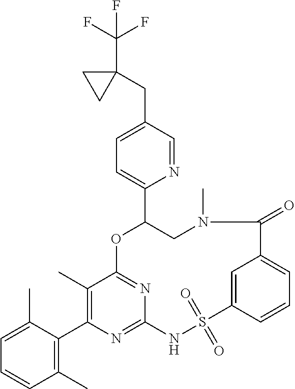 [SMILES: Cc1cccc(C)c1-c1nc2nc(c1C)OC(c1ccc(CC3(C(F)(F)F)CC3)cn1)CN(C)C(=O)c1cccc(c1)S(=O)(=O)N2] | | | | | +++ | +++ |

Table 21 represents CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” % Activity: +++ is >600%; ++ is 30-60%; + is <30%).

TABLE 21

| | | HBE | HBE Max | HBE |
|---|---|---|---|---|
| Compound | | EC₅₀ | Activity | Activity at |
| Number | Structure | (μM) | (%) | 10 μM (%) |
| 49 | 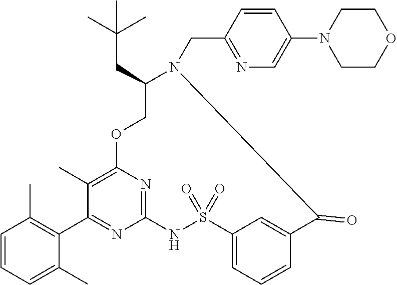 [SMILES: Cc1cccc(C)c1-c1nc2nc(c1C)OC[C@@H](CC(C)(C)C)N(Cc1ccc(N3CCOCC3)cn1)C(=O)c1cccc(c1)S(=O)(=O)N2] | +++ | +++ | |
| 56 | 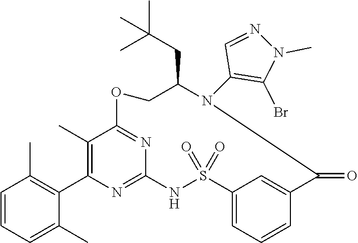 [SMILES: Cc1cccc(C)c1-c1nc2nc(c1C)OC[C@@H](CC(C)(C)C)N(c1cnn(C)c1Br)C(=O)c1cccc(c1)S(=O)(=O)N2] | +++ | +++ | |
| 85 | 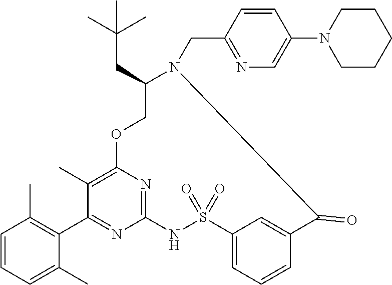 [SMILES: Cc1cccc(C)c1-c1nc2nc(c1C)OC[C@@H](CC(C)(C)C)N(Cc1ccc(N3CCCCC3)cn1)C(=O)c1cccc(c1)S(=O)(=O)N2] | +++ | +++ | |
| 173 | 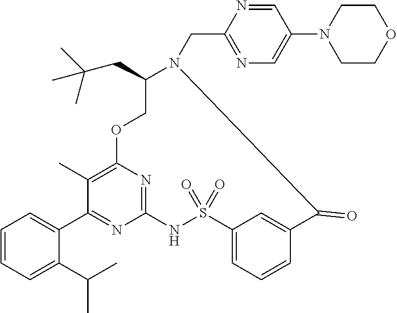 [SMILES: Cc1c2nc(nc1-c1ccccc1C(C)C)NS(=O)(=O)c1cccc(c1)C(=O)N(Cc1ncc(N3CCOCC3)cn1)[C@H](CC(C)(C)C)CO2] | +++ | +++ | |
| 181 | 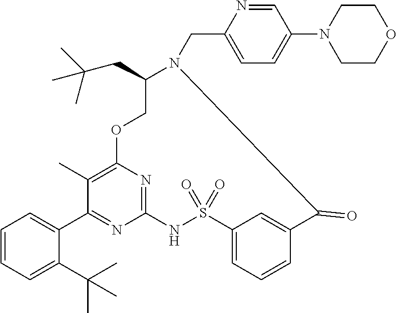 [SMILES: Cc1c2nc(nc1-c1ccccc1C(C)(C)C)NS(=O)(=O)c1cccc(c1)C(=O)N(Cc1ccc(N3CCOCC3)cn1)[C@H](CC(C)(C)C)CO2] | +++ | +++ | |
| 182 | 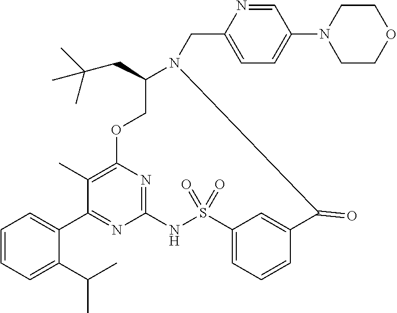 [SMILES: Cc1c2nc(nc1-c1ccccc1C(C)C)NS(=O)(=O)c1cccc(c1)C(=O)N(Cc1ccc(N3CCOCC3)cn1)[C@H](CC(C)(C)C)CO2] | +++ | +++ | |
| 191 | 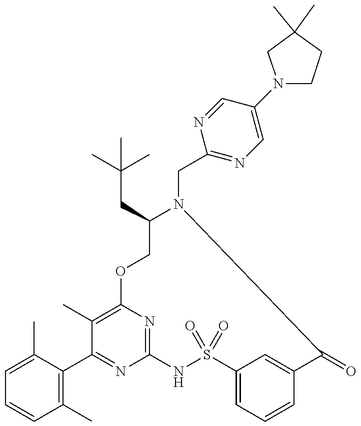 [SMILES: Cc1cccc(C)c1-c1nc2nc(c1C)OC[C@@H](CC(C)(C)C)N(Cc1ncc(N3CCC(C)(C)C3)cn1)C(=O)c1cccc(c1)S(=O)(=O)N2] | +++ | +++ | |
| 192 | 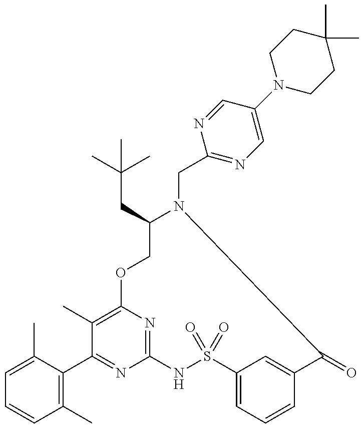 [SMILES: Cc1cccc(C)c1-c1nc2nc(c1C)OC[C@@H](CC(C)(C)C)N(Cc1ncc(N3CCC(C)(C)CC3)cn1)C(=O)c1cccc(c1)S(=O)(=O)N2] | | | +++ |
| 193 | 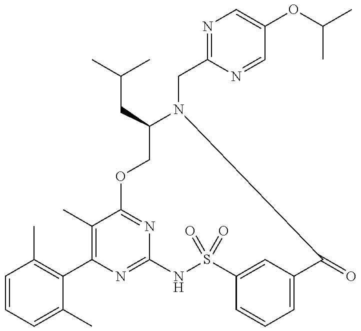 [SMILES: Cc1cccc(C)c1-c1nc2nc(c1C)OC[C@@H](CC(C)C)N(Cc1ncc(OC(C)C)cn1)C(=O)c1cccc(c1)S(=O)(=O)N2] | +++ | +++ | |
| 194 | 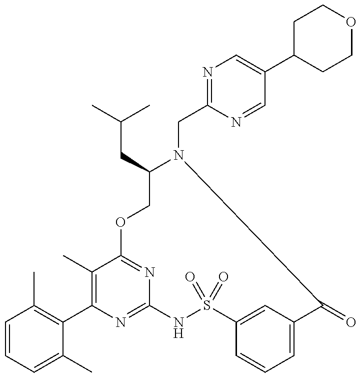 [SMILES: Cc1cccc(C)c1-c1nc2nc(c1C)OC[C@@H](CC(C)C)N(Cc1ncc(C3CCOCC3)cn1)C(=O)c1cccc(c1)S(=O)(=O)N2] | | | +++ |
| 195 | 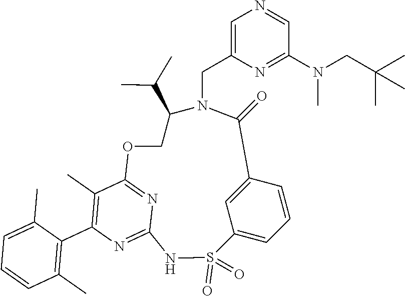 [SMILES: Cc1cccc(C)c1-c1nc2nc(c1C)OC[C@@H](C(C)C)N(Cc1cncc(N(C)CC(C)(C)C)n1)C(=O)c1cccc(c1)S(=O)(=O)N2] | +++ | +++ | |
| 196 | 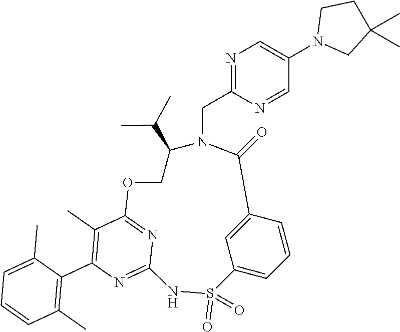 [SMILES: Cc1cccc(C)c1-c1nc2nc(c1C)OC[C@@H](C(C)C)N(Cc1ncc(N3CCC(C)(C)C3)cn1)C(=O)c1cccc(c1)S(=O)(=O)N2] | | | +++ |
| 197 | 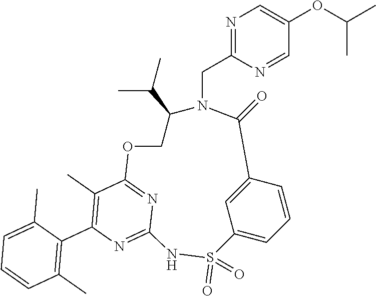 [SMILES: Cc1cccc(C)c1-c1nc2nc(c1C)OC[C@@H](C(C)C)N(Cc1ncc(OC(C)C)cn1)C(=O)c1cccc(c1)S(=O)(=O)N2] | | | +++ |
| 198 | 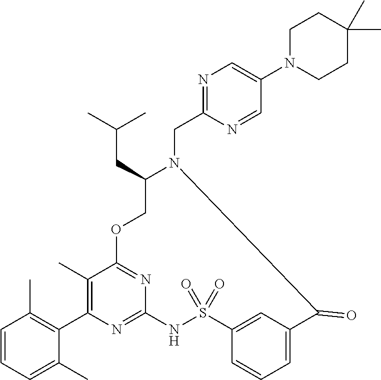 [SMILES: Cc1cccc(C)c1-c1nc2nc(c1C)OC[C@@H](CC(C)C)N(Cc1ncc(N3CCC(C)(C)CC3)cn1)C(=O)c1cccc(c1)S(=O)(=O)N2] | +++ | +++ | |
| 199 | 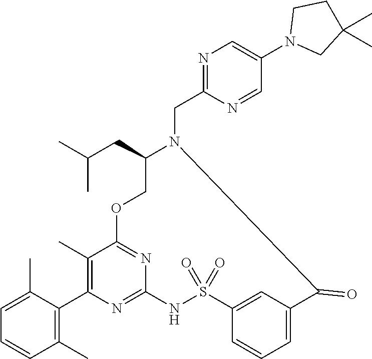 [SMILES: Cc1cccc(C)c1-c1nc2nc(c1C)OC[C@@H](CC(C)C)N(Cc1ncc(N3CCC(C)(C)C3)cn1)C(=O)c1cccc(c1)S(=O)(=O)N2] | +++ | +++ | |
| 200 | 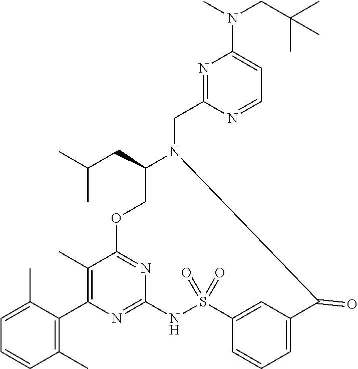 [SMILES: Cc1cccc(C)c1-c1nc2nc(c1C)OC[C@@H](CC(C)C)N(Cc1nccc(N(C)CC(C)(C)C)n1)C(=O)c1cccc(c1)S(=O)(=O)N2] | +++ | +++ | |
| 206 | 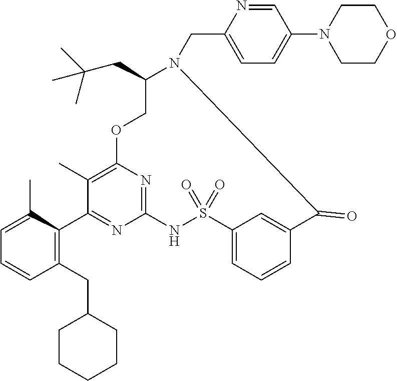 | +++ | +++ | |
| 207 | 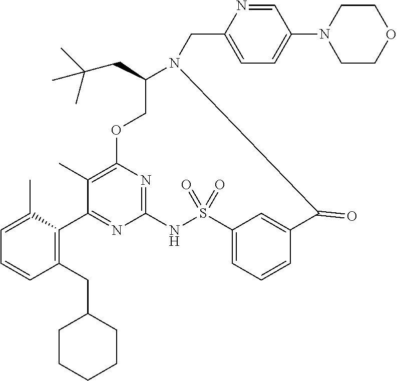 | +++ | +++ | |
| 208 | 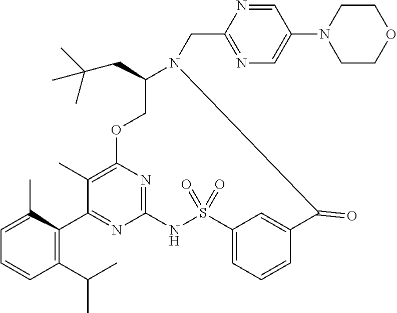 | +++ | +++ | |
| 209 | 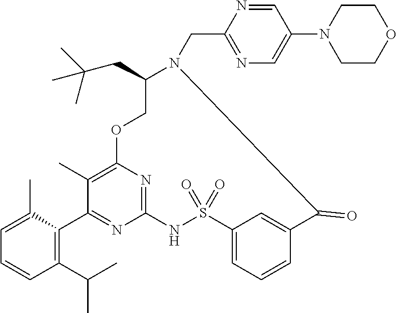 | +++ | +++ | |
| 210 | 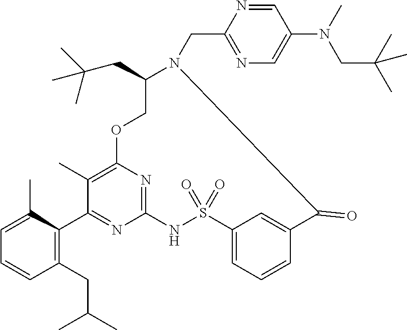 | | | +++ |
| 211 | 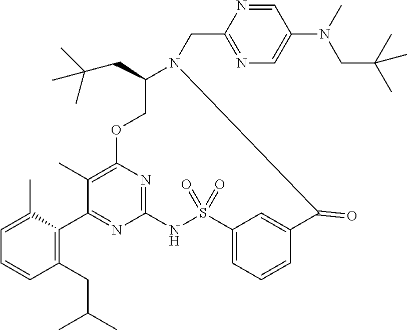 | +++ | +++ | |
| 212 | 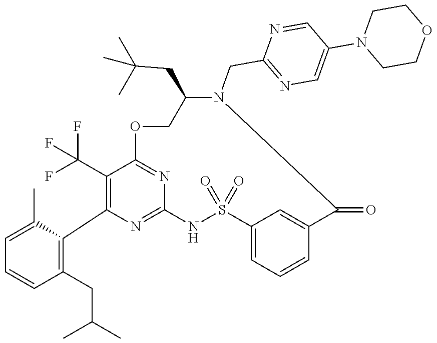 | +++ | +++ | |
| 213 | 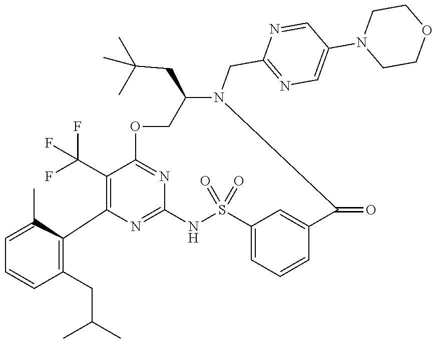 | | | +++ |
| 214 | 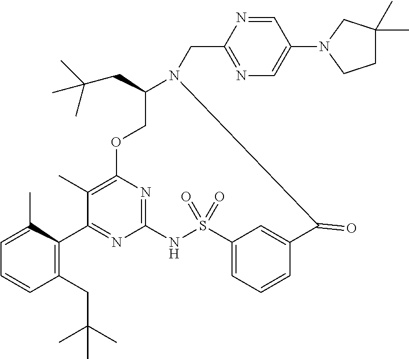 | | | ++ |
| 215 | 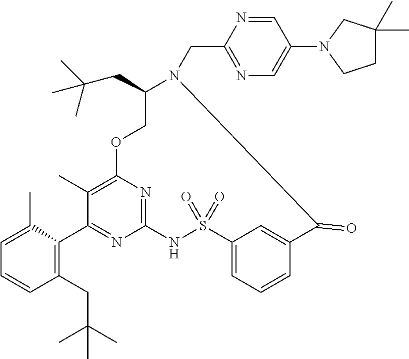 | +++ | +++ | |
| 216 | 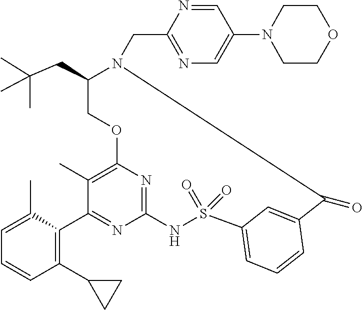 | +++ | +++ | |
| 217 | 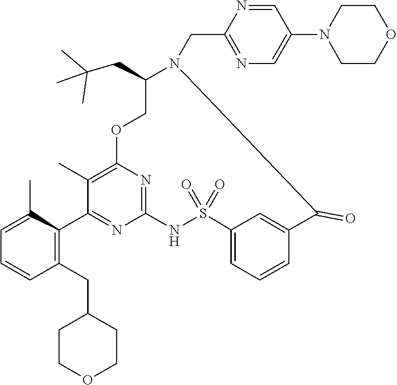 | | | ++ |
| 218 | 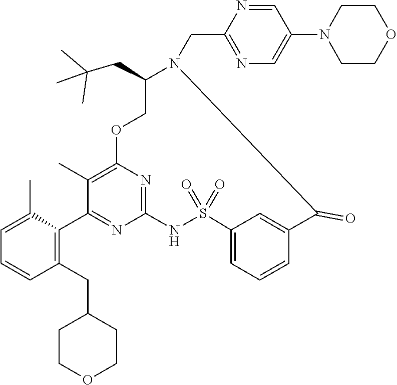 | +++ | +++ | |
| 219 | 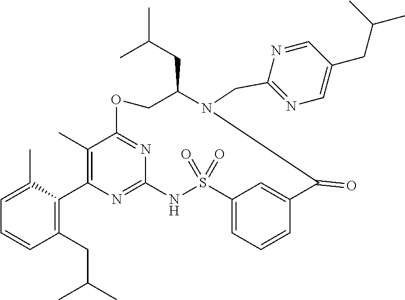 | +++ | +++ | |
| 220 | 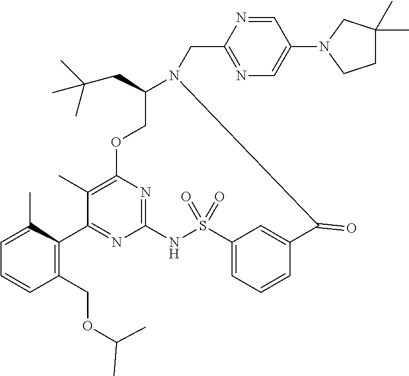 | | | ++ |
| 221 | 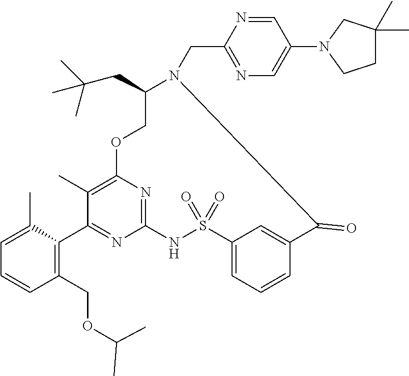 | +++ | +++ | |
| 222 | 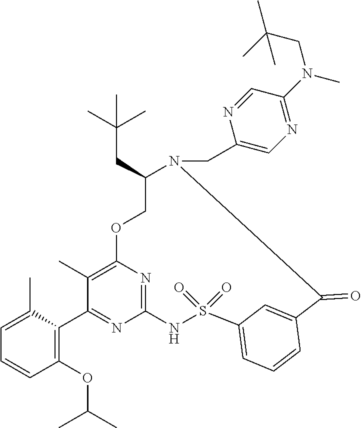 | | | +++ |
| 223 | 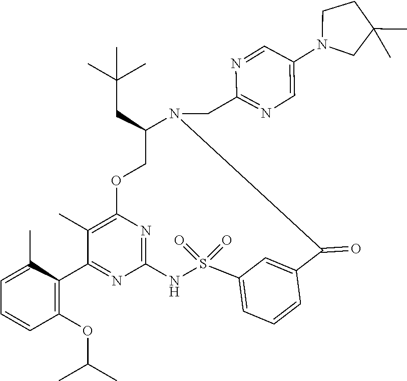 | | | +++ |
| 224 | 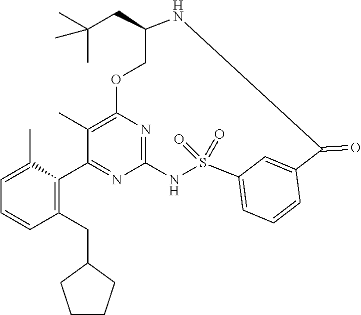 | | | +++ |
| 225 | 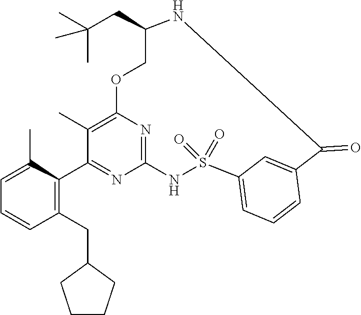 | | | +++ |
| 226 | 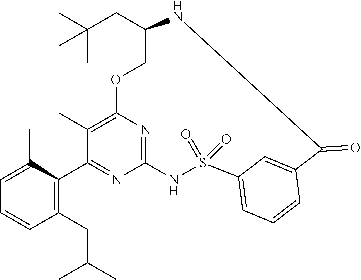 | | | +++ |
| 227 | 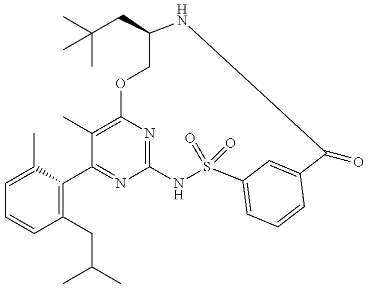 | | | +++ |
| 228 | 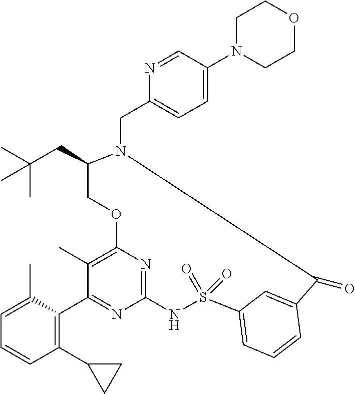 | +++ | +++ | |
| 229 | 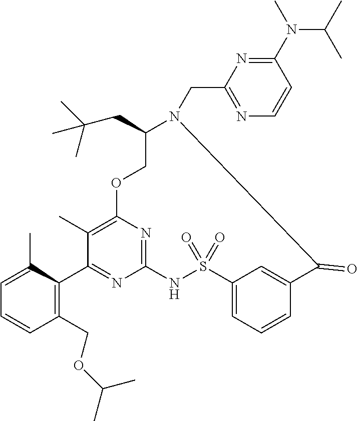 | | | +++ |
| 230 | 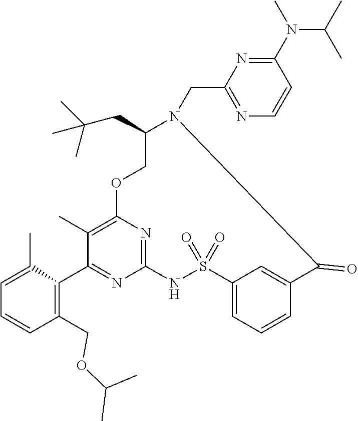 | | | +++ |
| 231 | 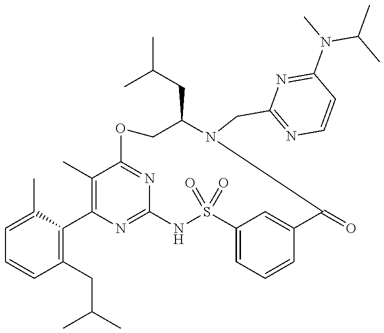 | | | +++ |
| 232 | 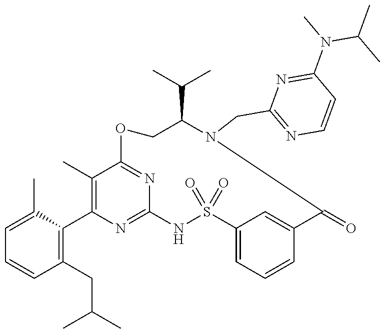 | | | +++ |
| 233 | 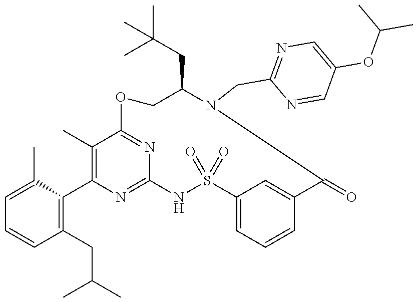 | +++ | +++ | |
| 234 | 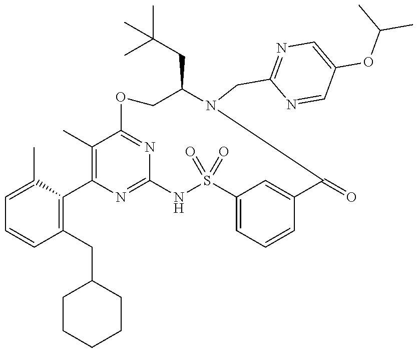 | +++ | +++ | |
| 235 | 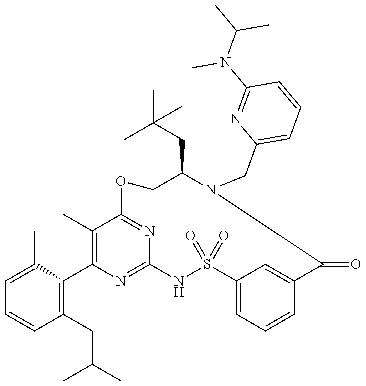 | +++ | +++ | |
| 236 | 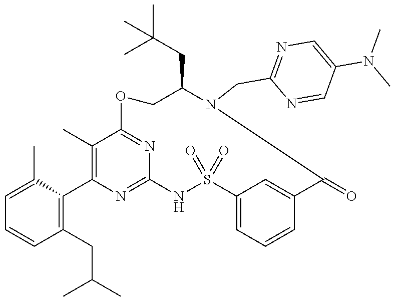 | +++ | +++ | |
| 237 | 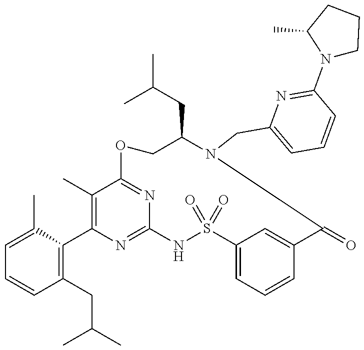 | +++ | +++ | |
| 238 | 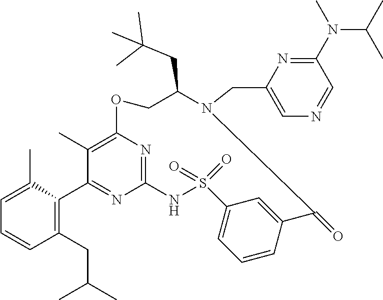 | +++ | +++ | |
| 239 | 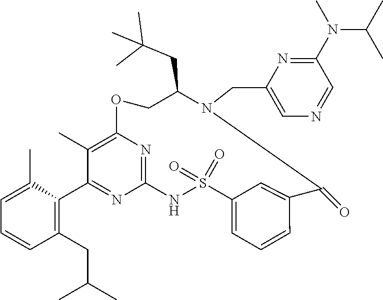 | +++ | +++ | |
| 240 | 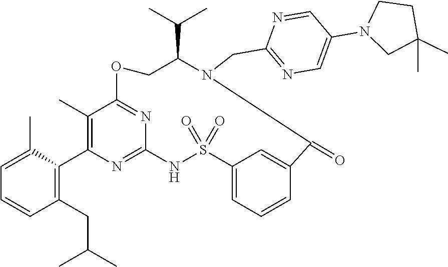 | ++ | +++ | |
| 241 | 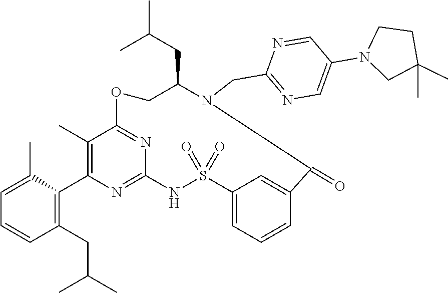 | +++ | +++ | |
| 242 | 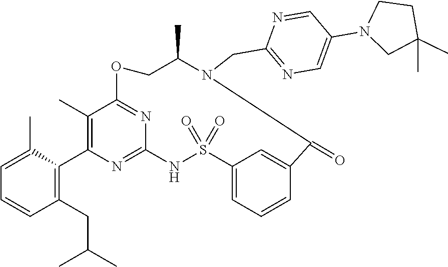 | +++ | +++ | |
| 243 | 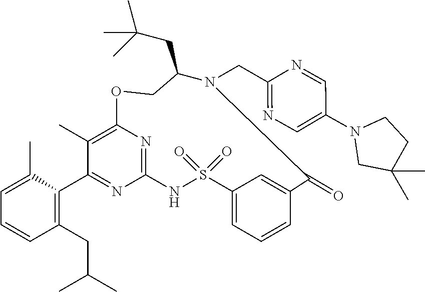 | ++ | +++ | |
| 244 | 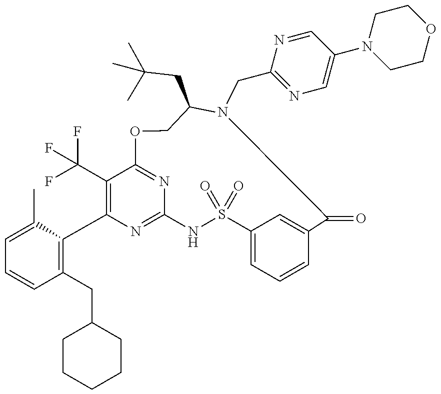 | +++ | +++ | |
| 245 | 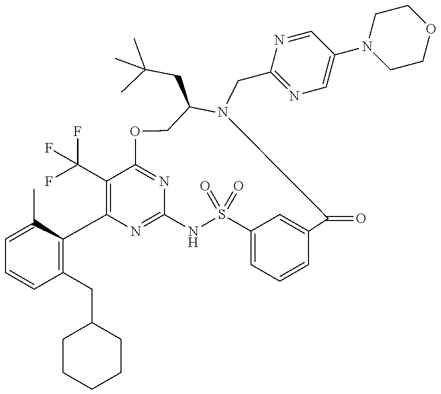 | | | ++ |
| 246 | 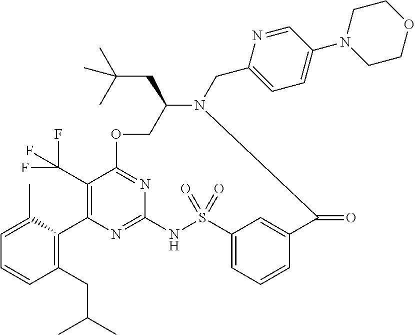 | +++ | +++ | |
| 247 | 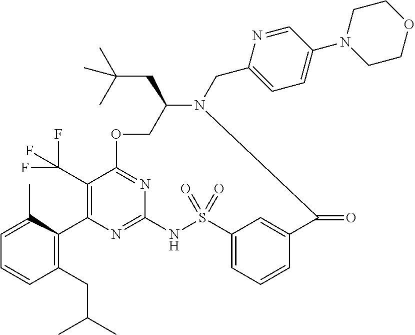 | | | ++ |
| 248 | 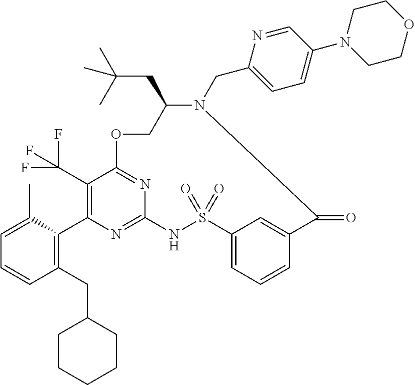 | | | +++ |
| 249 | 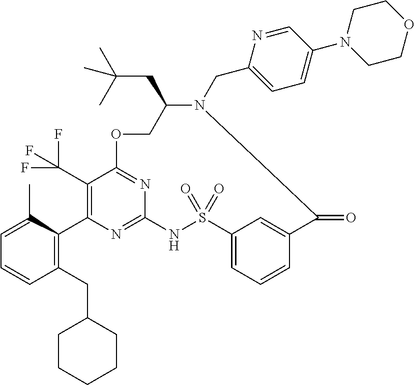 | | | ++ |
| 250 | 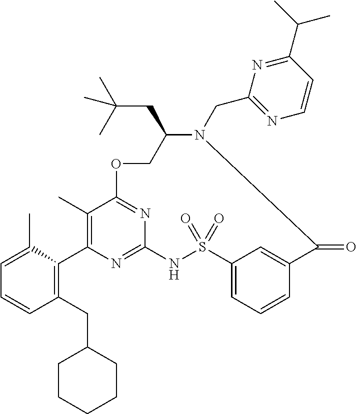 | +++ | +++ | |
| 251 | 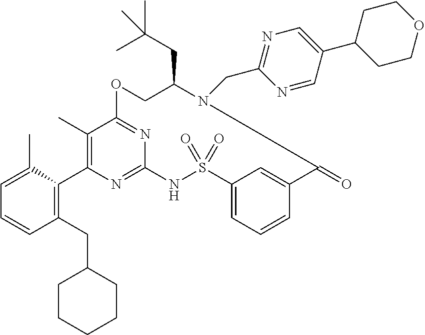 | +++ | +++ | |
| 252 | 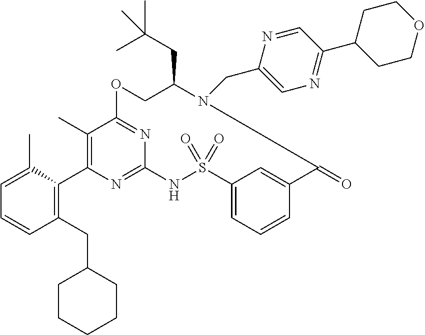 | +++ | +++ | |
| 253 | 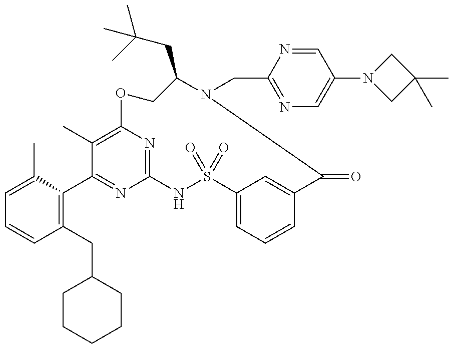 | +++ | +++ | |
| 254 | 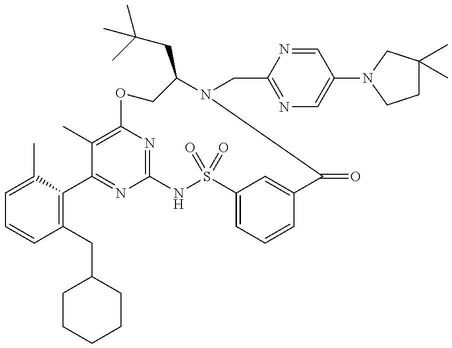 | ++ | +++ | |
| 255 | 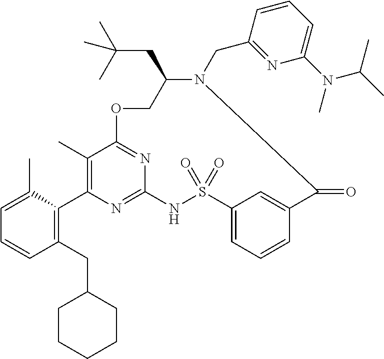 | +++ | +++ | |
| 256 | 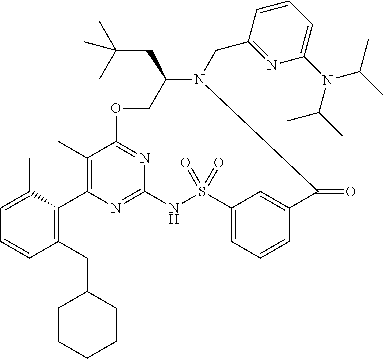 | +++ | +++ | |
| 257 | 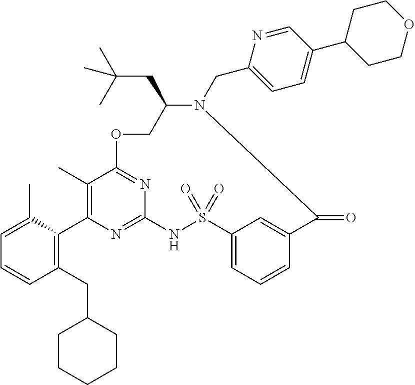 | +++ | +++ | |
| 258 | 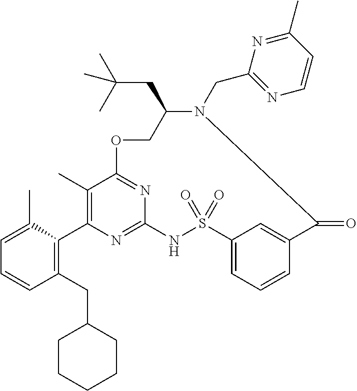 | +++ | +++ | |
| 259 | 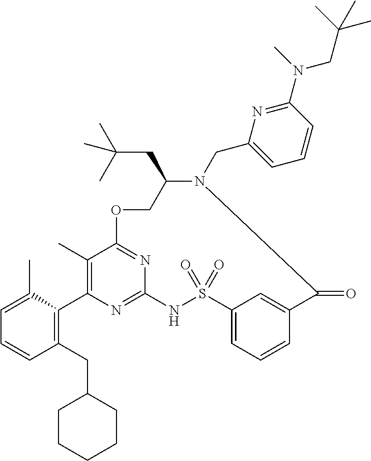 | +++ | +++ | |
| 260 | 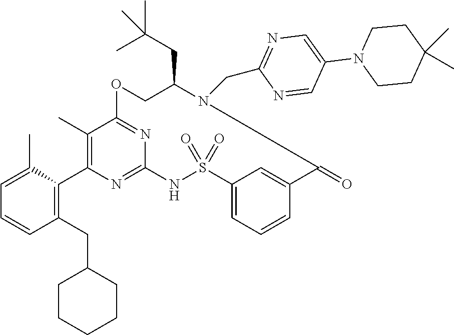 | +++ | +++ | |
| 261 | 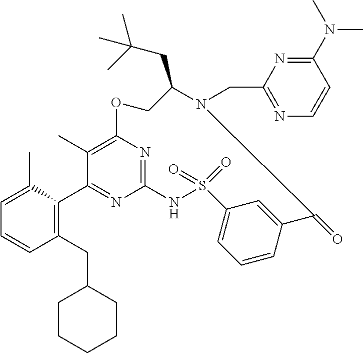 | +++ | +++ | |
| 262 | 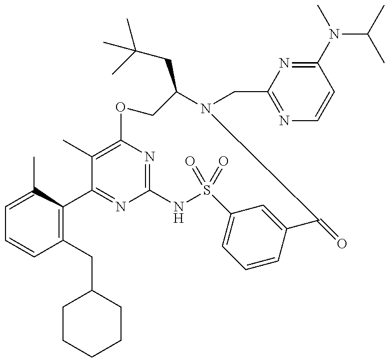 | +++ | +++ | |
| 263 | 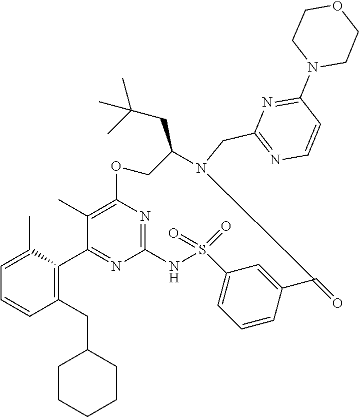 | +++ | +++ | |
| 264 | 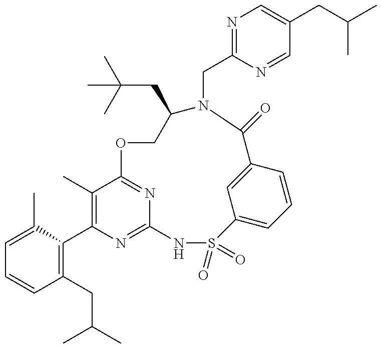 | +++ | +++ | |
| 265 | 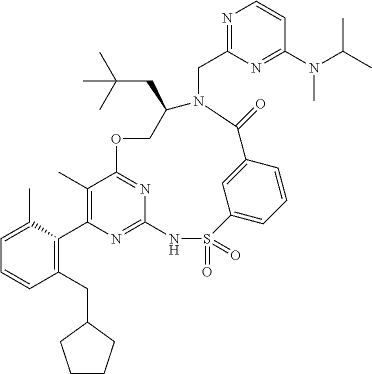 | | | +++ |
| 266 | 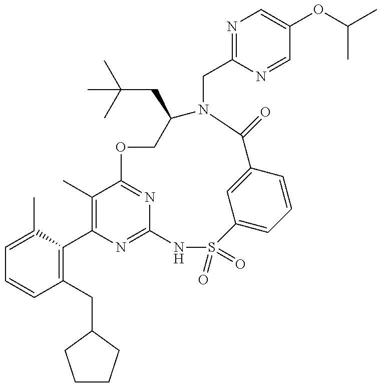 | +++ | +++ | |
| 267 | 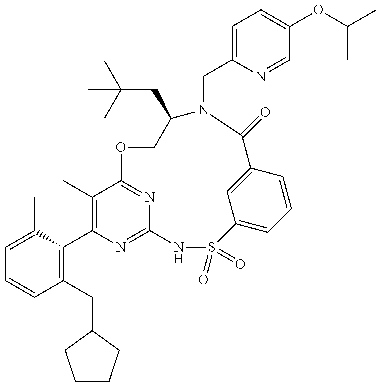 | +++ | +++ | |
| 268 | 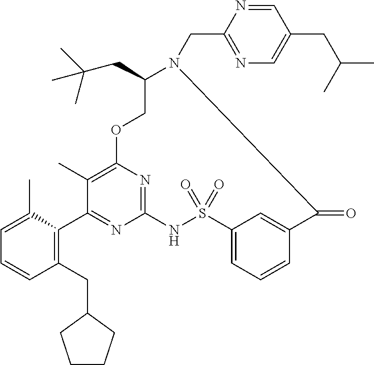 | +++ | +++ | |
| 269 | 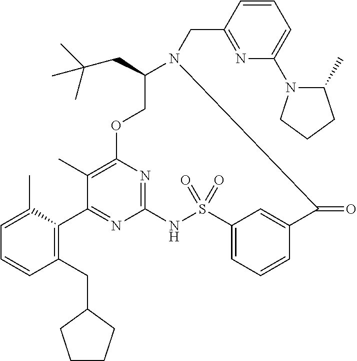 | | | +++ |
| 270 | 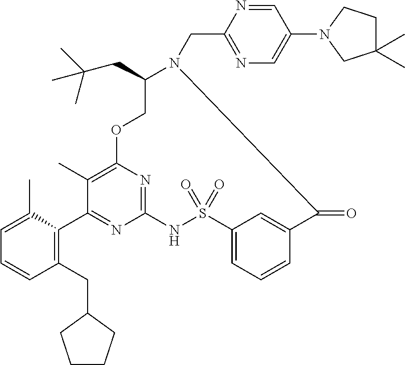 | | | +++ |
| 271 | 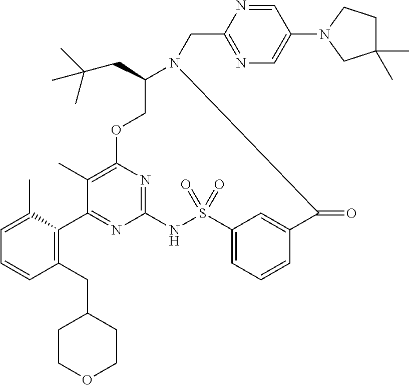 | | | +++ |
| 272 | 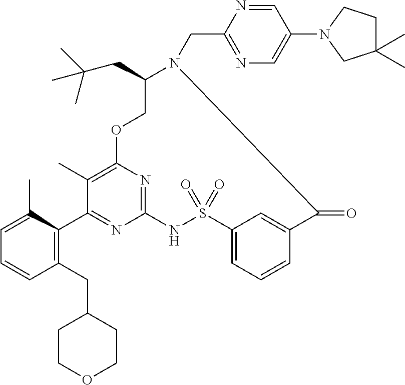 | | | ++ |
| 273 | 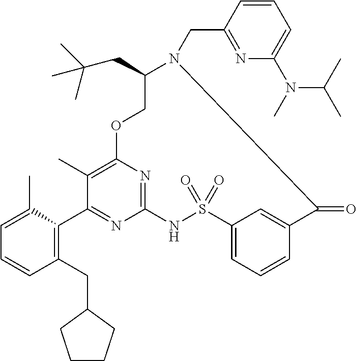 | +++ | +++ | |
| 274 | 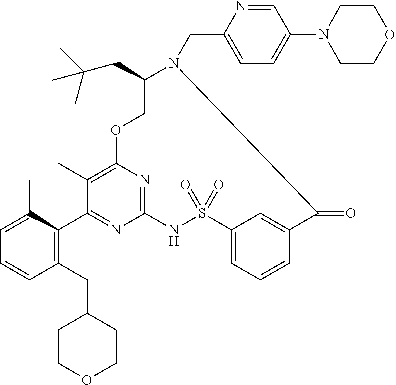 | | | ++ |
| 275 | 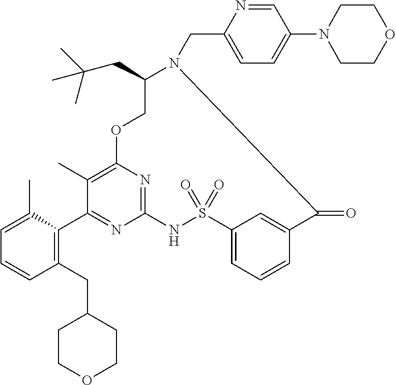 | | | +++ |
| 276 | 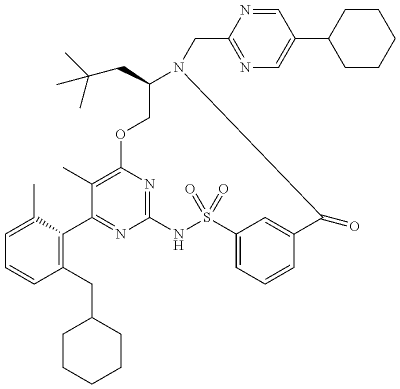 | +++ | +++ | |
| 277 | 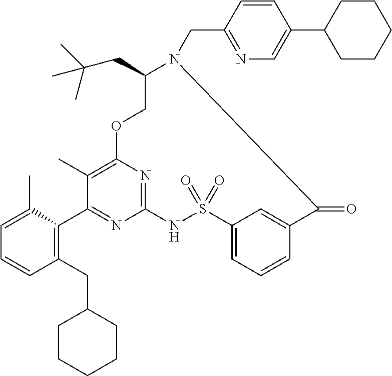 | +++ | +++ | |
| 278 | 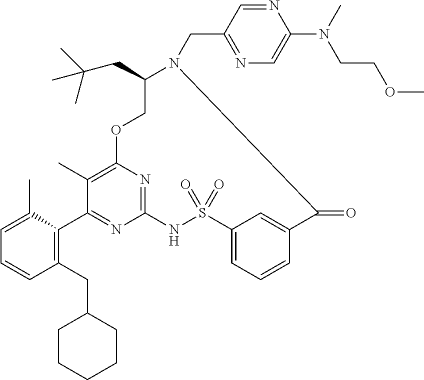 | ++ | +++ | |
| 279 | 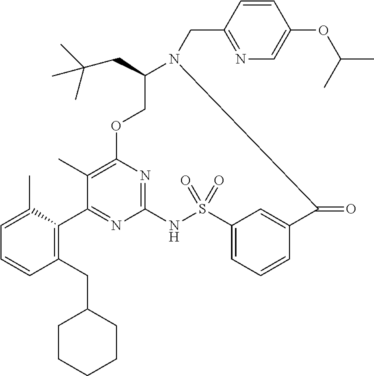 | ++ | +++ | |
| 280 | 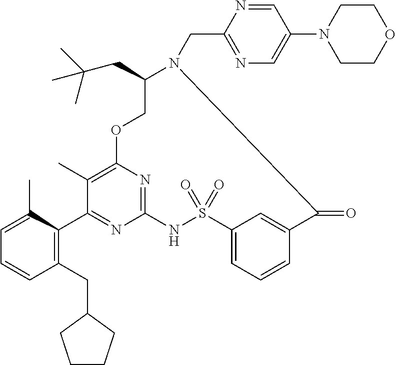 | +++ | +++ | |
| 281 | 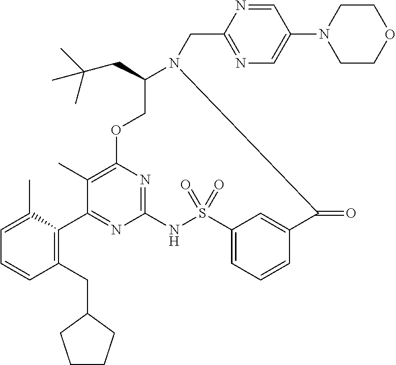 | +++ | +++ | |
| 282 | 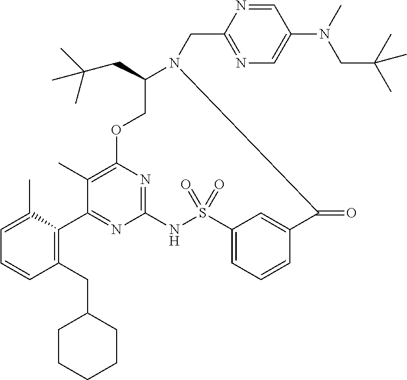 | ++ | +++ | |
| 283 | 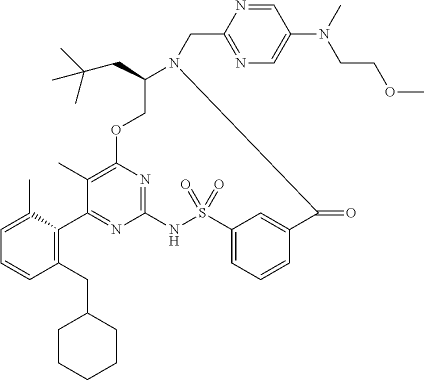 | +++ | +++ | |
| 284 | 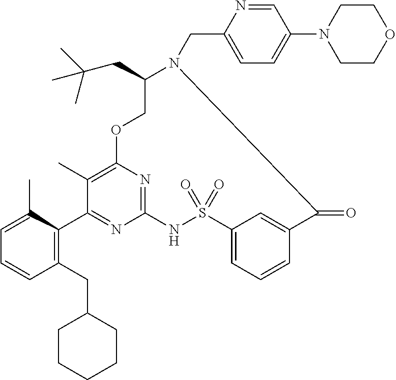 | | | +++ |
| 285 | 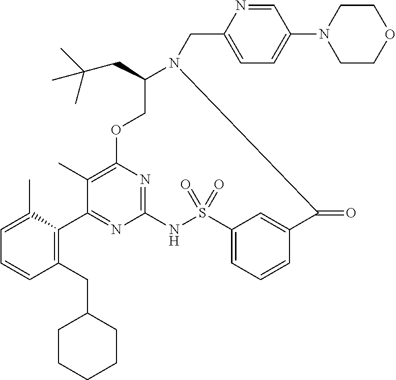 | +++ | +++ | |
| 286 | 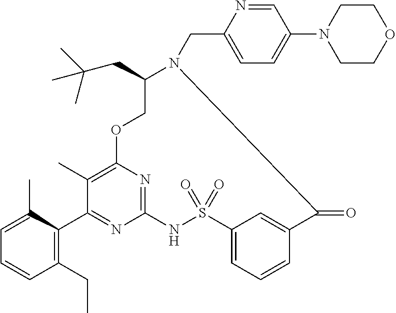 | +++ | +++ | |
| 287 | 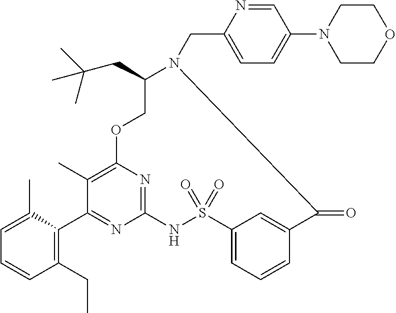 | +++ | +++ | |
| 288 | 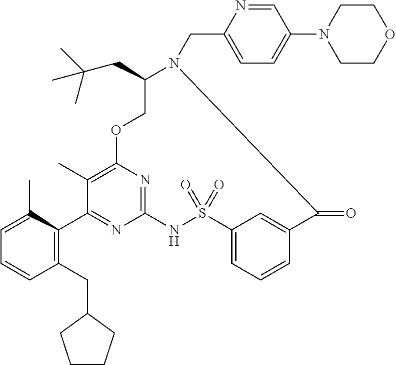 | | | +++ |
| 289 | 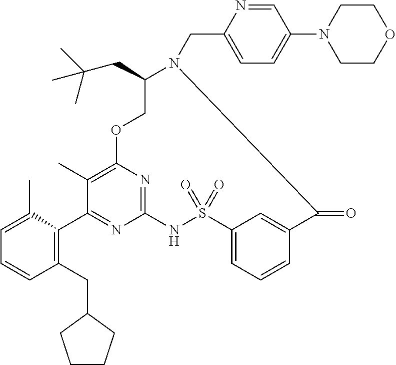 | +++ | +++ | |
| 290 | 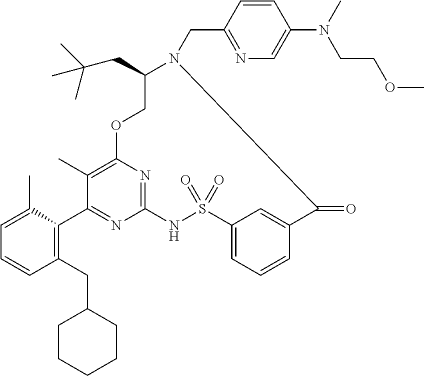 | +++ | +++ | |
| 291 | 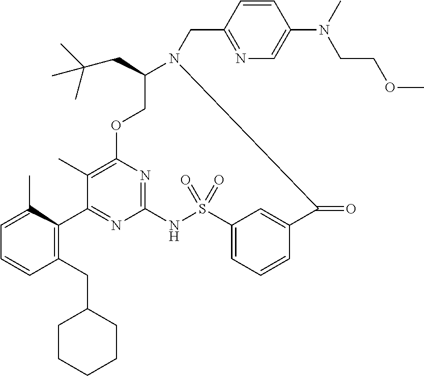 | | | +++ |
| 292 | 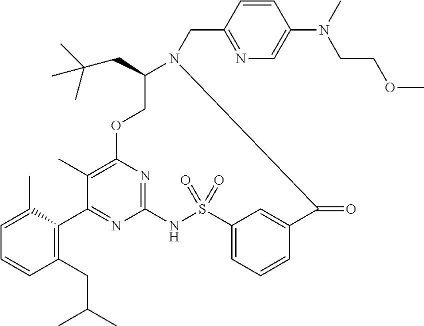 | +++ | +++ | |
| 293 | 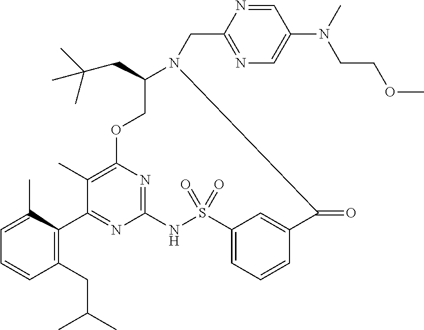 | | | +++ |
| 294 | 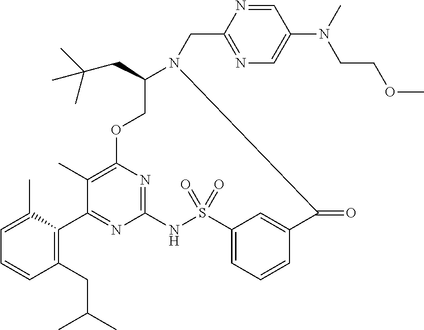 | +++ | +++ | |
| 295 | 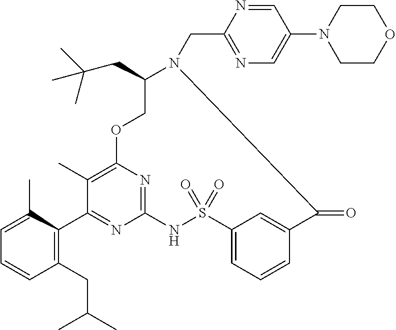 | +++ | +++ | |
| 296 | 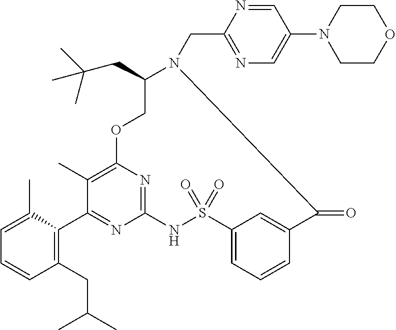 | +++ | +++ | |
| 297 | 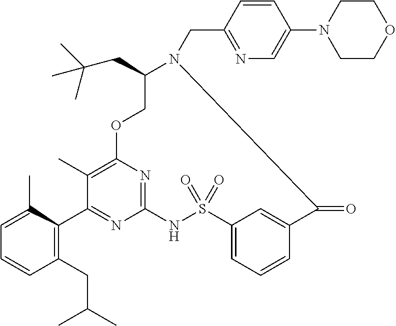 | +++ | +++ | |
| 298 | 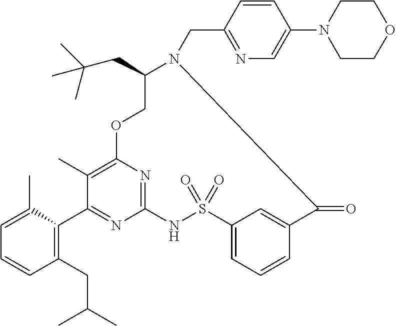 | +++ | +++ | |
| 299 | 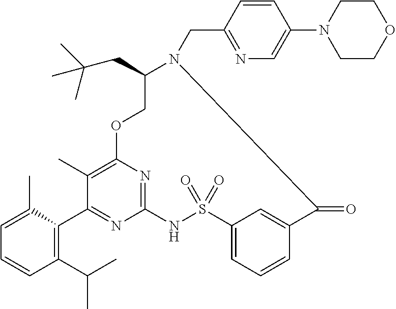 | +++ | +++ | |
| 300 | 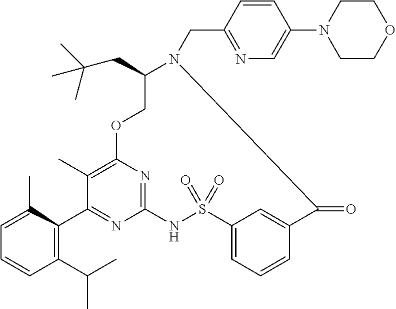 | +++ | +++ | |
| 301 | 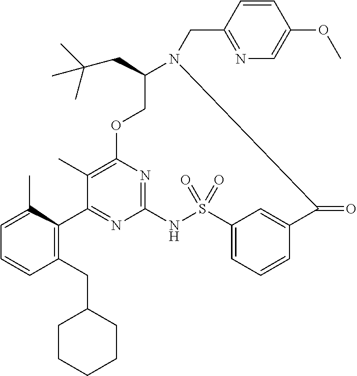 | +++ | +++ | |
| 302 | 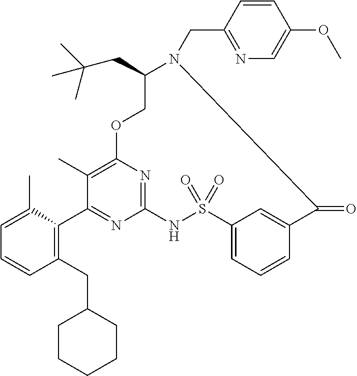 | +++ | +++ | |
| 303 | 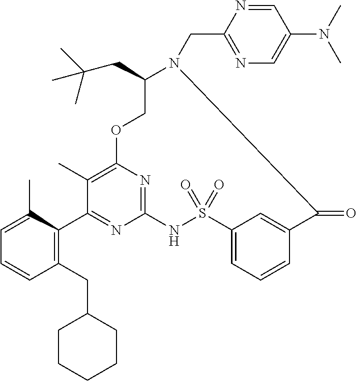 | +++ | +++ | |
| 304 | 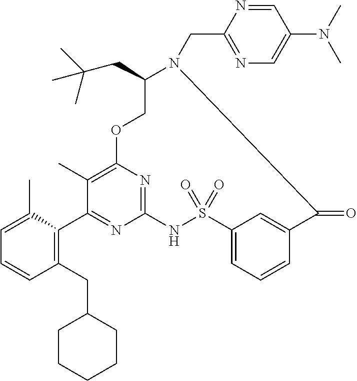 | +++ | +++ | |
| 305 | 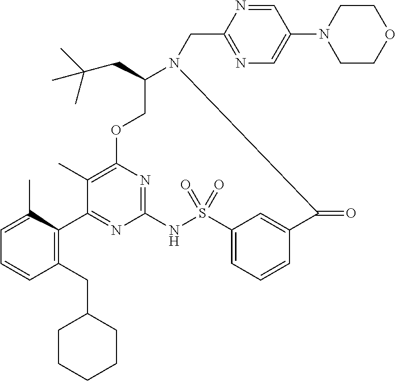 | +++ | +++ | |
| 306 | 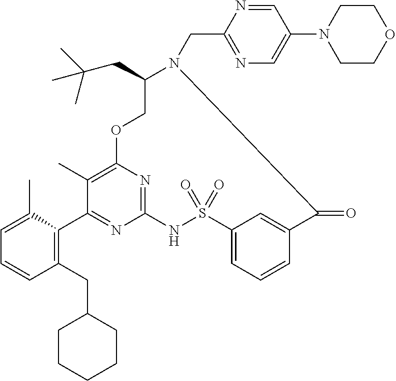 | +++ | +++ | |
| 307 | 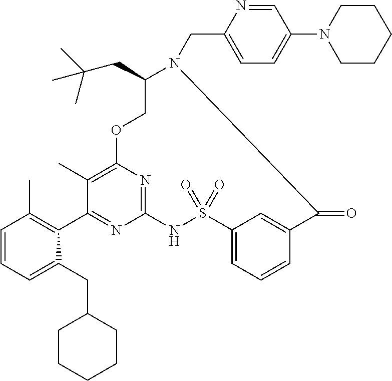 | +++ | +++ | |
| 308 | 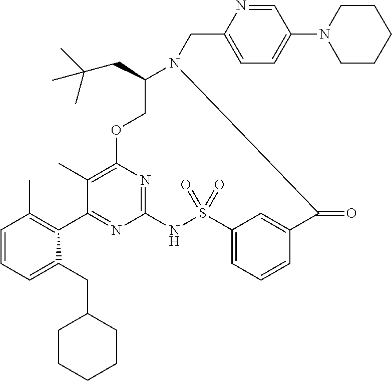 | +++ | +++ | |
| 309 | 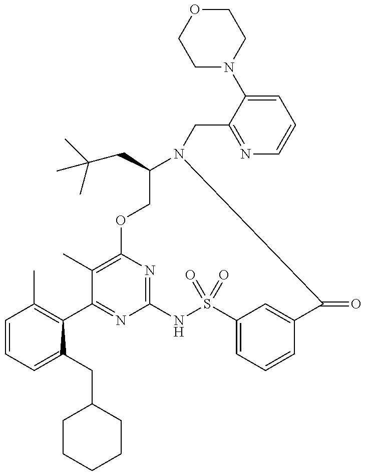 | +++ | +++ | |
| 310 | 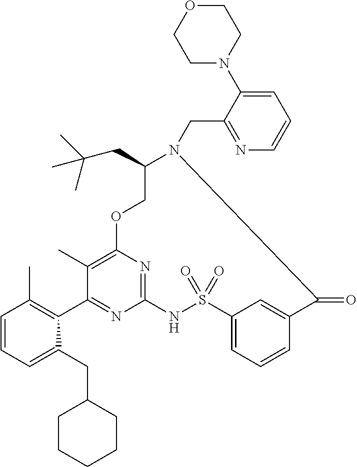 | +++ | +++ | |
| 311 | 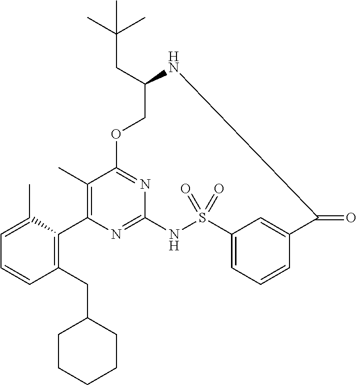 | +++ | +++ | |
| 312 | 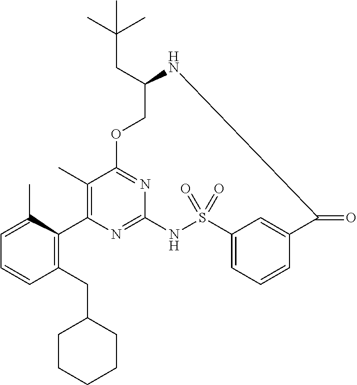 | +++ | ++ | |
| 313 | 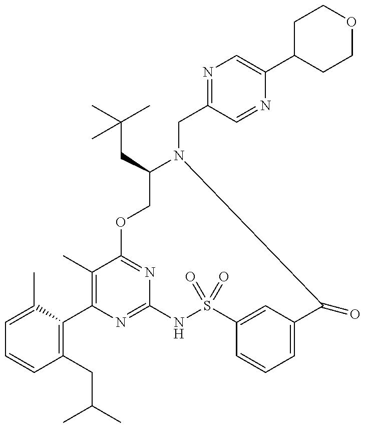 | +++ | +++ | |
| 314 | 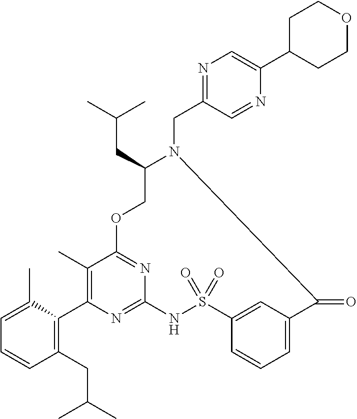 | +++ | +++ | |
| 315 | 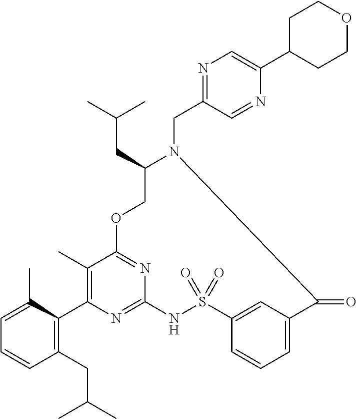 | | | +++ |
| 316 | 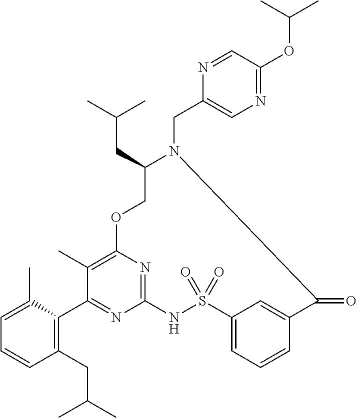 | +++ | +++ | |
| 317 | 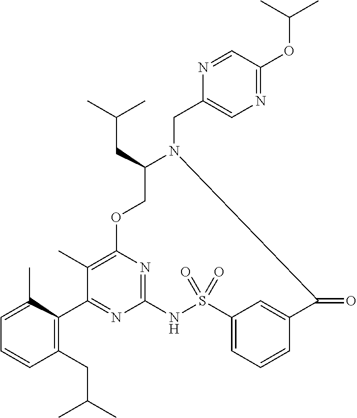 | | | +++ |
| 318 | 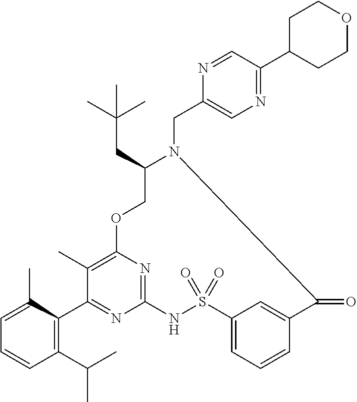 | | | +++ |
| 319 | 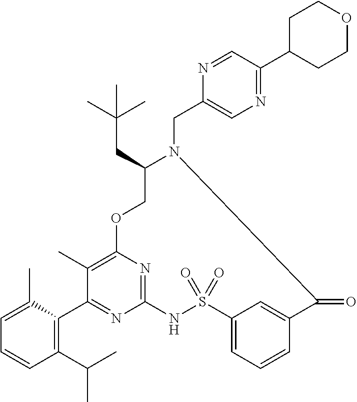 | | | +++ |
| 320 | 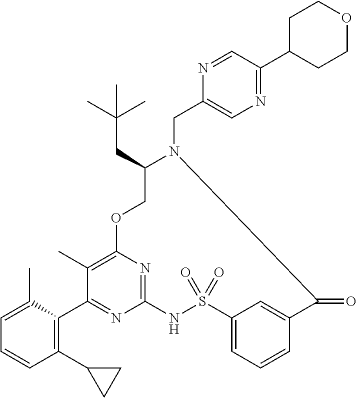 | +++ | +++ | |

### VI. Synthesis of Compounds 331-369

### A. Synthesis of common intermediates

### Preparation of 5-isopropoxypyrimidine-2-carbaldehyde

### Step 1: 2-Chloro-5-isopropoxy-pyrimidine

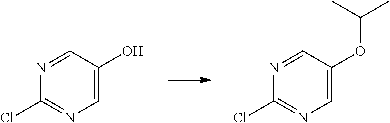

To a solution of 2-chloropyrimidin-5-ol (15 g, 114.91 mmol) in DMF (150 mL) was added potassium carbonate (32 g, 231.54 mmol) then 2-iodopropane (29.803 g, 17.5 mL, 175.32 mmol) and the mixture was heated at 50° C. for 1 h. The reaction was cooled to room temperature and diluted with Et₂O (1500 mL), washed with a mixture of brine (300 mL) and water (300 mL), then brine (2×300 mL) again. The organics were dried over sodium sulfate, filtered and concentrated in vacuo to provide 2-chloro-5-isopropoxy-pyrimidine (18.5 g, 91%) as flaky white crystals. ¹H NMR (500 MHz, Chloroform-d) δ 8.25 (s, 2H), 4.59 (hept, J=6.1 Hz, 1H), 1.38 (d, J=6.1 Hz, 6H). ESI-MS m/z calc. 172.04034, found 173.4 (M+1)⁺; Retention time: 2.2 minutes; LC method T.

### Step 2: Methyl 5-isopropoxypyrimidine-2-carboxylate

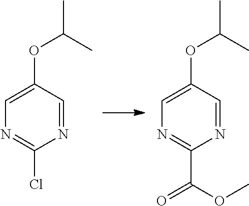

A stirred mixture of 2-chloro-5-isopropoxy-pyrimidine (40 g, 220.15 mmol) and Pd(dppf)₂Cl₂·DCM (10 g, 12.245 mmol) in DMF (200 mL)/MeOH (200 mL)/TEA (400.00 mL) in a 2 L steel bomb with an overhead mechanical stirrer was purged with carbon monoxide three times. The reaction mixture was heated to 120° C. with 120 psi of CO and retained at this temperature for one hour. Heating was turned off and the reaction mixture was allowed to cool to rt. Methanol and triethylamine were evaporated in vacuo. Water (1 L) was added and the suspension filtered to remove catalyst residue. The cake was washed with water. The filtrate was extracted with DCM (3×700 mL). The combined organic layer was washed with brine, dried over sodium sulfate, filtered and concentrated in vacuo. The resulting brown oil was purified by flash chromatography (SiO₂, 220 g, loaded in DCM, eluted with 25% ethyl acetate in hexanes) to give a greenish oil, which was triturated with hexanes and filtered. The cake was washed with hexanes and dried in vacuo to give methyl 5-isopropoxypyrimidine-2-carboxylate (30.1 g, 66%) as a beige solid. ESI-MS m/z calc. 196.0848, found 197.4 (M+1)⁺; Retention time: 3.15 minutes; LC method S.

### Step 3: 5-Isopropoxypyrimidine-2-carbaldehyde

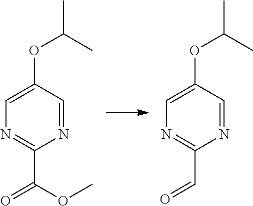

To a stirred solution of methyl 5-isopropoxypyrimidine-2-carboxylate (32.6 g, 157.85 mmol) in THF (1 L) was added DIBAL in toluene (240 mL of 1 M, 240.00 mmol) at −78° C. over 30 minutes and the reaction was stirred for 1 h. The reaction mixture was quenched with methanol (500 mL) and water (250 mL). The dry ice bath was removed, and the reaction mixture was allowed to warm to rt. The mixture was concentrated in vacuo to remove THF and methanol. DCM (2 L) was added, and the suspension was filtered. The organic layer from the filtrate was separated and the aqueous layer was extracted with DCM (2×1 L). The combined organic layer was dried over sodium sulfate, filtered, and concentrated in vacuo. The resulting yellow solid was triturated with hexanes and filtered. The cake was washed with hexanes and dried to give 5-isopropoxypyrimidine-2-carbaldehyde (23.3 g, 88%) as an off-white solid. ¹H NMR (500 MHz, DMSO-d₆) δ 9.89 (s, 1H), 8.75 (s, 2H), 4.99 (hept, J=6.0 Hz, 1H), 1.34 (d, J=6.0 Hz, 6H). ESI-MS m/z calc. 166.07423, found 167.2 (M+1)⁺; Retention time: 1.13 minutes; LC method W.

### Preparation of 6-[cyclobutyl(methyl)amino]pyrazine-2-carbaldehyde

### Step 1: Methyl 6-[cyclobutyl(methyl)amino]pyrazine-2-carboxylate

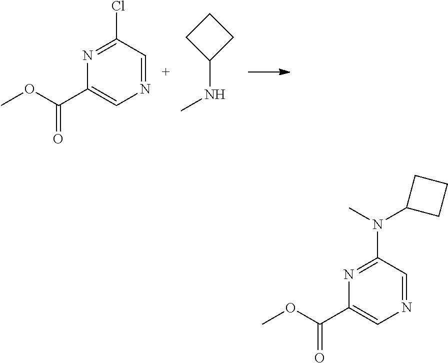

To a stirring solution of methyl 6-chloropyrazine-2-carboxylate (10.5 g, 60.845 mmol) and N-methylcyclobutanamine (hydrochloride salt) (9.46 g, 73.901 mmol) in anhydrous DMSO (150 mL) under nitrogen was added anhydrous sodium carbonate (20 g, 188.70 mmol) in one portion at room temperature. The resulting black mixture was stirred at 90° C. overnight. After cooling to room temperature, water (1000 mL) was added and the resulting solution was extracted with EtOAc (3×300 mL). The combined organic solutions were washed with water (2×300 mL), followed by brine (300 mL), dried over anhydrous sodium sulfate, and filtered. The solvent was removed by rotary evaporation and the residue was dried in vacuo overnight yielding methyl 6-[cyclobutyl(methyl) amino]pyrazine-2-carboxylate (7.77 g, 58%) as an amber oil. The crude was used in the next Step without further purification. ¹H NMR (500 MHz, CDCl₃) δ 8.48 (s, 1H), 8.14 (s, 1H), 4.76-4.65 (m, 1H), 3.96 (s, 3H), 3.11 (s, 3H), 2.37-2.27 (m, 2H), 2.26-2.14 (m, 2H), 1.82-1.70 (m, 2H). ESI-MS m/z calc. 221.11642, found 222.2 (M+1)⁺; Retention time: 2.43 minutes; LC method T.

### Step 2:[6-[Cyclobutyl(methyl)amino]pyrazin-2-yl]methanol

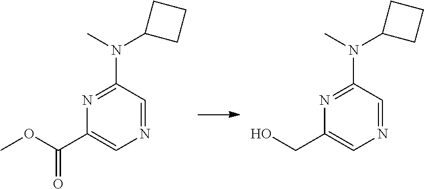 [SMILES: COC(=O)c1cncc(N(C)C2CCC2)n1 ; CN(c1cncc(CO)n1)C1CCC1]

A solution of methyl 6-[cyclobutyl(methyl)amino]pyrazine-2-carboxylate (7.77 g, 35.118 mmol) in MeOH (200 mL) was cooled to 0° C. using an ice bath. Sodium borohydride (13.3 g, 351.55 mmol) was then added in portions over 15 min at the same temperature. The reaction mixture was stirred for 1 h and then warmed to room temperature and stirred for 7 h. The reaction mixture was quenched with water (100 mL). MeOH was removed by rotary evaporation and the remaining aqueous layer was further diluted with water (200 mL), saturated with sodium chloride, and extracted with DCM (100 mL×5). The combined organic solutions were dried over anhydrous sodium sulfate and filtered. The solvent was removed by rotary evaporation and the residue was dried in vacuo for 5 h yielding [6-[cyclobutyl(methyl)amino]pyrazin-2-yl]methanol (5.45 g, 79%) as a yellow oil. The crude was used in the next Step without further purification. ESI-MS m/z calc. 193.1215, found 194.1 (M+1)⁺; Retention time: 1.41 minutes; LC method T.

### Step 3: 6-[Cyclobutyl(methyl)amino]pyrazine-2-carbaldehyde

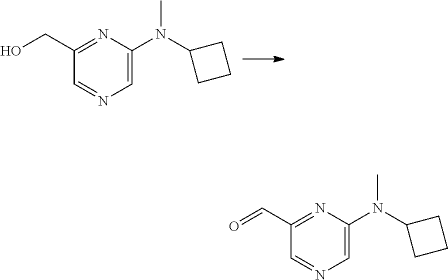

A solution of [6-[cyclobutyl(methyl)amino]pyrazin-2-yl]methanol (5.45 g, 27.638 mmol) in anhydrous DCM (250 mL) was added with DMP (14.2 g, 33.479 mmol) in portions over 5 min under nitrogen. The resulting amber solution was stirred at room temperature overnight. LCMS indicated incomplete oxidation. More DMP (3.6 g, 8.4877 mmol) was added and stirring was continued for 3 h. The reaction mixture was added with a saturated aqueous sodium bicarbonate solution (200 mL) and stirred for 15 min. The organic layer was separated and washed further with saturated aqueous sodium bicarbonate solution (2×200 mL). The organic layer was dried over anhydrous sodium sulfate and filtered. The solvent was removed by rotary evaporation and the crude aldehyde was purified by silica flash chromatography (330 g, dry loaded, eluting from 0 to 40% EtOAc in hexanes over a 70 min gradient). The fractions were combined and concentrated under reduced pressure and the residue was further dried in vacuo overnight yielding 6-[cyclobutyl(methyl)amino]pyrazine-2-carbaldehyde (3.7 g, 67%) as an orange liquid. ¹H NMR (500 MHz, DMSO-d₆) δ 9.90 (s, 1H), 8.40 (s, 1H), 8.27 (s, 1H), 4.89-4.80 (m, 1H), 3.08 (s, 3H), 2.26-2.16 (m, 4H), 1.75-1.61 (m, 2H). ESI-MS m/z calc. 191.10587, found 192.1 (M+1)⁺; Retention time: 2.19 minutes; LC method W.

### B. Synthesis of Compounds

### Preparation of Compound 364 and Compound 361

### Step 1: (11R)-6-(2,6-Dimethylphenyl)-11-(2,2-dimethylpropyl)-7-methyl-12-[1-methyl-5-(2-methylprop-1-enyl)pyrazol-4-yl]-2,2-dioxo-9-oxa-2λ⁶-thia-3,5,12,19-tetrazatricyclo[12.3.1.14,8]nonadeca-1(18),4(19),5,7,14,16-hexaen-13-one

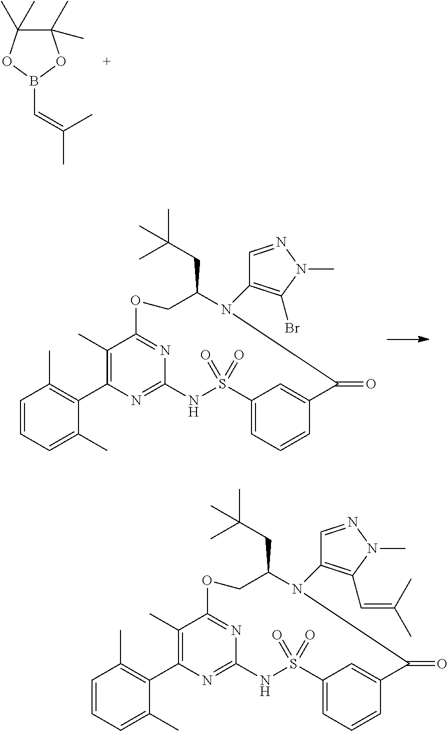 [SMILES: CC(C)=CB1OC(C)(C)C(C)(C)O1 ; Cc1cccc(C)c1-c1nc2nc(c1C)OC[C@@H](CC(C)(C)C)N(c1cnn(C)c1Br)C(=O)c1cccc(c1)S(=O)(=O)N2 ; CC(C)=Cc1c(N2C(=O)c3cccc(c3)S(=O)(=O)Nc3nc(c(C)c(-c4c(C)cccc4C)n3)OC[C@H]2CC(C)(C)C)cnn1C]

A 10 mL tube was charged with (11R)-12-(5-bromo-1-methyl-pyrazol-4-yl)-6-(2,6-dimethylphenyl)-11-(2,2-dimethylpropyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (20 mg, 0.0289 mmol), potassium carbonate (12 mg, 0.0868 mmol), dioxane (0.5 mL) and water (0.13 mL). The mixture was purged with nitrogen for 5 minutes and then 4,4,5,5-tetramethyl-2-(2-methylprop-1-enyl)-1,3,2-dioxaborolane (7.1032 mg, 8 μL, 0.0390 mmol) and 1,1′-bis(diphenylphosphino) ferrocene palladium(II) chloride, complex with dichloromethane (2.5 mg, 0.0031 mmol) were added. The mixture was purged with nitrogen for 5 minutes and the tube was sealed. The reaction mixture was stirred at 100° C. for 20 hours. The reaction mixture was diluted with water (5 mL) and extracted with DCM (2×5 mL). The combined organic phases were dried over sodium sulfate, filtered and concentrated to dryness. The residue was dissolved in dioxane (1 mL) and water (0.25 mL) and the mixture was purged with nitrogen for 10 minutes. 4,4,5,5-Tetramethyl-2-(2-methylprop-1-enyl)-1,3,2-dioxaborolane (8.8700 mg, 10 μL, 0.0487 mmol), potassium carbonate (12 mg, 0.0868 mmol) and 1,1′-bis(diphenylphosphino)ferrocene palladium(II) chloride, complex with dichloromethane (3.5 mg, 0.0043 mmol) were added and the mixture was purged with nitrogen for 5 minutes. The tube was sealed, and the mixture was stirred at 100° C. for 24 hours. The reaction mixture was diluted with water (10 mL) and extracted with DCM (2×10 mL). The combined organic phases were dried over sodium sulfate, filtered and concentrated in vacuo. Purification by reverse phase chromatography (Column: 15.5 g Cis. Gradient: 5-100% MeCN in water with 0.1% formic acid) afforded (11R)-6-(2,6-dimethylphenyl)-11-(2,2-dimethylpropyl)-7-methyl-12-[1-methyl-5-(2-methylprop-1-enyl)pyrazol-4-yl]-2,2-dioxo-9-oxa-2λ⁶-thia-3,5,12,19-tetrazatricyclo[12.3.1.14,8]nonadeca-1(18),4(19),5,7,14,16-hexaen-13-one (6.52 mg, 35%) as an off-white solid after lyophilization. ¹H NMR (400 MHz, DMSO-d₆) δ 13.03 (br. s., 1H), 8.69-8.59 (m, 1H), 8.03-7.91 (m, 1H), 7.70 (br. s., 2H), 7.58 (s, 1H), 7.33-7.21 (m, 1H), 7.20-7.06 (m, 2H), 6.00 (s, 1H), 5.28 (br. s., 1H), 4.01-3.81 (m, 2H), 3.71 (s, 3H), 2.05 (br. s., 3H), 1.96 (d, J=1.0 Hz, 3H), 1.75 (br. s., 3H), 1.64-1.52 (m, 7H), 1.17 (d, J=15.9 Hz, 1H), 0.49 (s, 9H). ESI-MS m/z calc. 642.2988, found 643.3 (M+1)⁺; Retention time: 4.42 minutes; LC method Y.

### Step 2: (11R)-6-(2,6-Dimethylphenyl)-11-(2,2-dimethylpropyl)-12-(5-isobutyl-1-methyl-pyrazol-4-yl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one

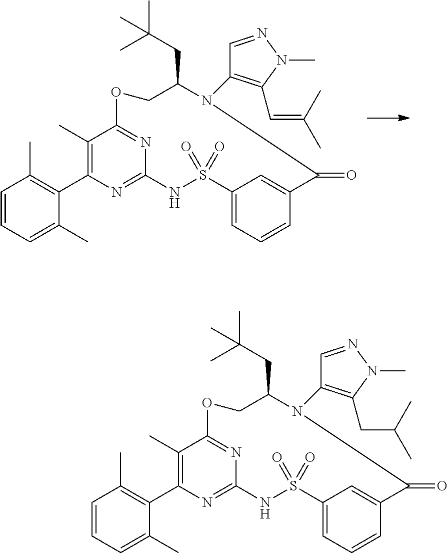 [SMILES: CC(C)=Cc1c(N2C(=O)c3cccc(c3)S(=O)(=O)Nc3nc(c(C)c(-c4c(C)cccc4C)n3)OC[C@H]2CC(C)(C)C)cnn1C ; Cc1cccc(C)c1-c1nc2nc(c1C)OC[C@@H](CC(C)(C)C)N(c1cnn(C)c1CC(C)C)C(=O)c1cccc(c1)S(=O)(=O)N2]

To a solution of (11R)-6-(2,6-dimethylphenyl)-11-(2,2-dimethylpropyl)-7-methyl-12-[1-methyl-5-(2-methylprop-1-enyl)pyrazol-4-yl]-2,2-dioxo-9-oxa-2λ⁶-thia-3,5,12,19-tetrazatricyclo[12.3.1.14,8]nonadeca-1(18),4(19),5,7,14,16-hexaen-13-one (41 mg, 0.0637 mmol) in degassed MeOH (3 mL) was added 10% palladium on carbon 50% wet (13.6 mg, 0.0064 mmol) and the mixture was purged with nitrogen for 5 minutes. Hydrogen was bubbled into the solution for 10 minutes and then the mixture was stirred at room temperature for 4 hours under a hydrogen atmosphere (1 atmosphere, balloon). The mixture was filtered over a Celite pad, the pad was rinsed with MeOH (10 mL) and the filtrate was concentrated in vacuo. The residue was dissolved in degassed MeOH (3 mL), 10% palladium on carbon 50% wet (24 mg, 0.0113 mmol) was added and the mixture was purged with nitrogen for 5 minutes. Hydrogen was bubbled into the solution for 10 minutes and then the mixture was stirred at room temperature for 18 hours under a hydrogen atmosphere (1 atmosphere, balloon). The mixture was filtered over a Celite pad, the pad was rinsed with MeOH (10 mL) and the filtrate was concentrated in vacuo. The residue was dissolved in degassed MeOH (3 mL), 10% palladium on carbon 50% wet (40 mg, 0.0188 mmol) was added, the mixture was purged with nitrogen for 5 minutes and then stirred under a hydrogen atmosphere (50 psi) for 4 hours at room temperature and at 50° C. for 16 hours. The mixture was filtered over a Celite pad, the pad was rinsed with MeOH (10 mL) and the filtrate was concentrated in vacuo. The residue was dissolved in degassed MeOH (3 mL) and THF (1 mL), 20% palladium hydroxide on carbon 50% wet (223 mg, 0.1588 mmol) was added and the mixture was purged with nitrogen for 5 minutes. The mixture was stirred under a hydrogen atmosphere (80 psi) for 5 days at room temperature. The mixture was filtered over a Celite pad, the pad was rinsed with MeOH (10 mL) and the filtrate was concentrated in vacuo. Purification by reverse phase chromatography (Column: 15.5 g Cis. Gradient: 5-100% MeCN in water with 0.1% formic acid), by SFC using a Lux Cellulose 4 column (250×21.2 mm), 5 uM, 40° C.; mobile phase: 30% MeOH (0.1% DEA), 70% CO₂, flow: 75 mL/min, concentration: 4.5 mg/mL in MeOH, injection volume 1.2 mL, 100 bar and then by reverse phase chromatography (Column: 5.5 g Cis. Gradient: 5-100% MeOH in water) afforded (11R)-6-(2,6-dimethylphenyl)-11-(2,2-dimethylpropyl)-12-(5-isobutyl-1-methyl-pyrazol-4-yl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (5.95 mg, 14%) as a white solid after lyophilization. ¹H NMR (400 MHz, DMSO-d₆) δ 13.05 (br. s., 1H), 8.67 (br. s., 1H), 7.98 (br. s., 1H), 7.81-7.62 (m, 2H), 7.51 (s, 1H), 7.33-7.23 (m, 1H), 7.17 (d, J=6.4 Hz, 1H), 7.12 (d, J=7.1 Hz, 1H), 5.39 (br. s., 1H), 4.07-3.96 (m, 1H), 3.93-3.85 (m, 1H), 3.82 (s, 3H), 2.66-2.58 (m, 1H), 2.57-2.52 (m, 1H, overlapped with DMSO), 2.05 (br. s., 3H), 1.93 (br. s., 1H), 1.83-1.66 (m, 4H), 1.56 (br. s., 3H), 1.25-1.19 (m, 1H), 0.92 (d, J=6.4 Hz, 3H), 0.86 (d, J=6.6 Hz, 3H), 0.55 (s, 9H). ESI-MS m/z calc. 644.31445, found 645.2 (M+1)⁺; Retention time: 4.39 minutes; LC method Y.

### Preparation of Compound 363

### Step 1: 2,4-Dichloro-1-oxido-3-(trifluoromethyl)pyridin-1-ium

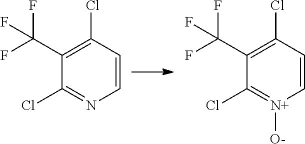 [SMILES: FC(F)(F)c1c(Cl)ccnc1Cl ; [O-][n+]1ccc(Cl)c(C(F)(F)F)c1Cl]

A flame-dried flask was charged with 2,4-dichloro-3-(trifluoromethyl)pyridine (10 g, 46.299 mmol) and anhydrous dichloromethane (200 mL). The solution was then cooled down to 0° C. and urea hydrogen peroxide (9.2 g, 97.800 mmol) was added. The reaction was stirred for 5 minutes at 0° C. then trifluoroacetic anhydride (21.605 g, 14.5 mL, 102.87 mmol) was added dropwise. The reaction was stirred for 15 minutes at 0° C. then warmed up to room temperature and stirred overnight at room temperature under nitrogen atmosphere. The reaction was cooled down to 0° C. and quenched by the addition of cold water (200 mL). Powdered sodium carbonate was then added portionwise until no bubbling (final pH around 8) and the two layers were separated. The aqueous layers was extracted with dichloromethane (4×200 mL) and the combined organic extracts were washed with brine (800 mL), dried over magnesium sulfate, filtered and concentrated under reduced pressure to afford 2,4-dichloro-1-oxido-3-(trifluoromethyl)pyridin-1-ium (9.775 g, 89%) as an off-white crystalline powder which was directly used in the next Step without further purification. ¹H NMR (400 MHz, DMSO-d₆) δ 8.69 (d, J=7.1 Hz, 1H), 7.79 (d, J=7.1 Hz, 1H).19F NMR (377 MHz, DMSO-d₆) δ −56.41 (s, 3F). ESI-MS m/z calc. 230.94655, found 232.0 (M+1)⁺; Retention time: 1.49 minutes; LC method X.

### Step 2: 4,6-Dichloro-5-(trifluoromethyl)pyridin-2-amine

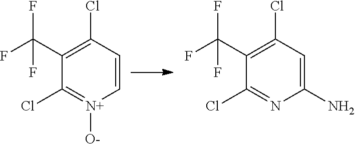 [SMILES: [O-][n+]1ccc(Cl)c(C(F)(F)F)c1Cl ; Nc1cc(Cl)c(C(F)(F)F)c(Cl)n1]

To a stirred solution of 2,4-dichloro-1-oxido-3-(trifluoromethyl)pyridin-1-ium (22.78 g, 98.195 mmol) in dry acetonitrile (250 mL) under nitrogen atmosphere at 0° C. were added methanesulfonic anhydride (26 g, 149.26 mmol) and pyridine (20.538 g, 21 mL, 259.65 mmol). The reaction mixture was warmed up to room temperature then stirred at room temperature for 1h. The reaction was then cooled down to 0° C. then ethanolamine (14.168 g, 14 mL, 231.95 mmol) was added. The reaction mixture was warmed up to room temperature then stirred at room temperature for 1h. LCMS indicated no product formation. Supplemental ethanolamine (14.168 g, 14 mL, 231.95 mmol) was added and the reaction mixture was stirred for another hour. Water (500 mL) and dichloromethane (500 mL) were added. The layers were separated and the aqueous layer was extracted with dichloromethane (4×100 mL). The combined organic layers were washed with brine (500 mL), dried over anhydrous sodium sulfate, filtered, and concentrated under reduced pressure. The crude product was purified by silica flash chromatography in 2 batches (330 g, dry loaded, eluting from 0 to 30% EtOAc in hexanes over a 70 min gradient). The correct fractions were combined and concentrated under reduced pressure and the residue was dried in vacuo yielding 4,6-dichloro-5-(trifluoromethyl)pyridin-2-amine (9.88 g, 44%) as a yellow solid. ESI-MS m/z calc. 229.96254, found 231.1 (M+1)⁺; Retention time: 2.8 minutes; LC method T.

### Step 3: 4-Chloro-6-(2,6-dimethylphenyl)-5-(trifluoromethyl)pyridin-2-amine

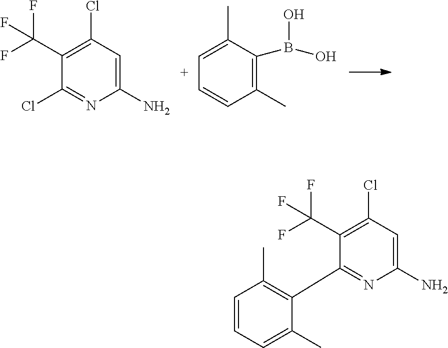 [SMILES: Nc1cc(Cl)c(C(F)(F)F)c(Cl)n1 ; Cc1cccc(C)c1B(O)O ; Cc1cccc(C)c1-c1nc(N)cc(Cl)c1C(F)(F)F]

A mixture of 4,6-dichloro-5-(trifluoromethyl)pyridin-2-amine (5.91 g, 25.584 mmol), (2,6-dimethylphenyl)boronic acid (11.52 g, 76.809 mmol) and sodium carbonate (13.56 g, 127.94 mmol) in water (100 mL) and DME (100 mL) was bubbled with nitrogen for 10 minutes. Pd(PPh3)4 (8.90 g, 7.7019 mmol) was added to the reaction mixture. The reaction mixture was heated to 100° C. overnight. LCMS showed formation of the desired product (same retention time as the starting material). After cooling to room temperature, the reaction mixture was diluted with ethyl acetate (200 mL) and water (200 mL). Layers were separated and the aqueous layer was extracted with ethyl acetate (3×100 mL). The combined organic layers were dried over anhydrous sodium sulfate and concentrated under vacuum. The residue was purified by silica gel chromatography (330 g, dry loaded, eluting from 0 to 35% EtOAc in hexanes over a 65 min gradient). The correct fractions were concentrated and further purified by silica gel chromatography using 0 to 100% DCM in hexanes over 65 min yielding 4-chloro-6-(2,6-dimethylphenyl)-5-(trifluoromethyl)pyridin-2-amine (973.8 mg, 12%) as a white solid. ¹H NMR (500 MHz, DMSO-d₆) δ 7.17-7.08 (m, 3H), 7.05 (d, J=7.6 Hz, 2H), 6.67 (s, 1H), 1.94 (s, 6H). ESI-MS m/z calc. 300.06412, found 301.3 (M+1)⁺; Retention time: 2.37 minutes; LC method W.

### Step 4: 6-Amino-2-(2,6-dimethylphenyl)-3-(trifluoromethyl)pyridin-4-ol

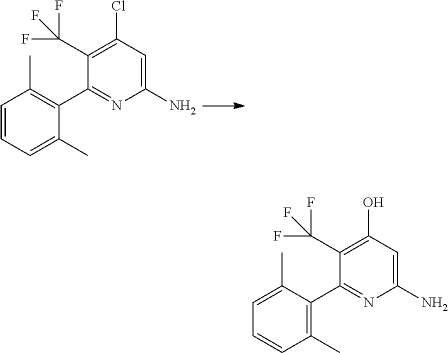 [SMILES: Cc1cccc(C)c1-c1nc(N)cc(Cl)c1C(F)(F)F ; Cc1cccc(C)c1-c1nc(N)cc(O)c1C(F)(F)F]

A tube was flame-dried then cooled down to room temperature using a nitrogen stream. The tube was charged with 4-chloro-6-(2,6-dimethylphenyl)-5-(trifluoromethyl)pyridin-2-amine (500 mg, 1.6228 mmol), benzaldehyde oxime (270 mg, 2.1174 mmol), freshly ground cesium carbonate (1.6 g, 4.9107 mmol) and anhydrous DMF (5 mL). Nitrogen was bubbled for 10 minutes after which time RockPhos Pd G3 (70 mg, 0.0835 mmol) was added. The tube was sealed and the reaction was stirred overnight at 80° C. The reaction was cooled down to room temperature, filtered on a pad of celite, washed with ethyl acetate (3×20 mL). The filtrate was concentrated under reduced pressure then purified by reverse-phase chromatography on Cis (column: 30 g HP Gold C₁₈, gradient: 5 to 100% acetonitrile in in 10 mM ammonium bicarbonate buffer pH=10, 20 CV). The desired fractions were concentrated under reduced pressure then the residual water was co-evaporated with methanol (6×10 mL) and acetonitrile (5×15 mL). The off-white residue was freeze-dried to afford 6-amino-2-(2,6-dimethylphenyl)-3-(trifluoromethyl)pyridin-4-ol (243 mg, 45%) as an off-white powder. ESI-MS m/z calc. 282.098, found 283.1 (M+1)⁺; Retention time: 2.49 minutes; LC method 1D.

### Step 5: tert-Butyl N-[(1R)-1-[[6-amino-2-(2,6-dimethylphenyl)-3-(trifluoromethyl)-4-pyridyl]oxymethyl]-3,3-dimethyl-butyl]carbamate

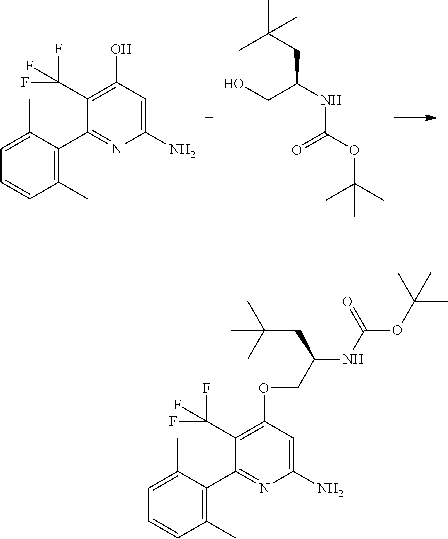 [SMILES: Cc1cccc(C)c1-c1nc(N)cc(O)c1C(F)(F)F ; CC(C)(C)C[C@H](CO)NC(=O)OC(C)(C)C ; Cc1cccc(C)c1-c1nc(N)cc(OC[C@@H](CC(C)(C)C)NC(=O)OC(C)(C)C)c1C(F)(F)F]

To stirred solution of tert-butyl N-[(1R)-1-(hydroxymethyl)-3,3-dimethyl-butyl]carbamate (550 mg, 2.1802 mmol) in anhydrous toluene (10 mL) under nitrogen atmosphere were added 6-amino-2-(2,6-dimethylphenyl)-3-(trifluoromethyl)pyridin-4-ol (243 mg, 0.7266 mmol) and triphenylphosphine (565 mg, 2.1541 mmol). The milky mixture was then cooled down to 0° C. and diisopropyl azodicarboxylate (442.90 mg, 430 μL, 2.1903 mmol) was added dropwise. The reaction was stirred for 30 minutes at 0° C. then stirred at room temperature for 2 days. The reaction was then concentrated under reduced pressure and purified by reverse-phase chromatography (column: 80 g Cis; gradient: 10 to 100% acetonitrile in 10 mM ammonium carbonate pH=10, 20 CV). The desired fractions were concentrated under reduced pressure and the remaining water was co-evaporated with methanol (5×10 mL), acetonitrile (5×5 mL) and the off-white residue was freeze-dried to afford tert-butyl N-[(1R)-1-[[6-amino-2-(2,6-dimethylphenyl)-3-(trifluoromethyl)-4-pyridyl]oxymethyl]-3,3-dimethyl-butyl]carbamate (290 mg, 78%) as an off-white powder ¹H NMR (400 MHz, DMSO-d₆) δ 7.15-7.05 (m, 1H), 7.03-6.99 (m, 2H), 6.79 (d, J=8.8 Hz, 1H), 6.60 (s, 2H), 6.09 (s, 1H), 3.91-3.82 (m, 2H), 3.77-3.70 (m, 1H), 1.94 (s, 3H), 1.93 (s, 3H), 1.41-1.36 (m, 11H), 0.90 (s, 9H).19F NMR (377 MHz, DMSO-d₆) δ −54.38 (s, 3F). ESI-MS m/z calc. 495.27087, found 496.2 (M+1)⁺; Retention time: 4.12 minutes; LC method 1D.

### Step 6: 3-[[4-[(2R)-2-(tert-Butoxycarbonylamino)-4,4-dimethyl-pentoxy]-6-(2,6-dimethylphenyl)-5-(trifluoromethyl)-2-pyridyl]sulfamoyl]benzoic acid

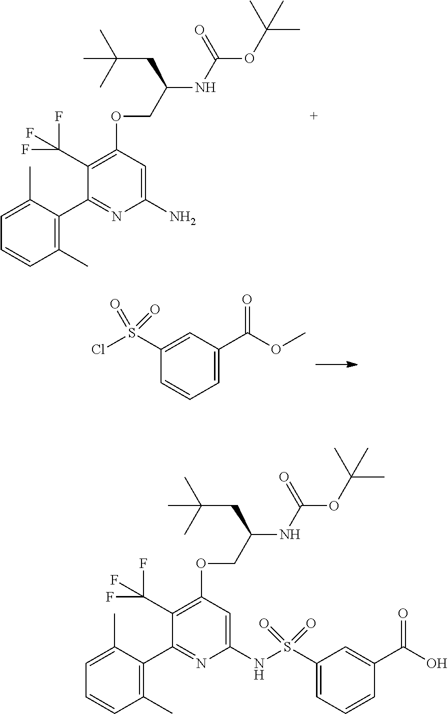

A stirred solution of tert-butyl N-[(1R)-1-[[6-amino-2-(2,6-dimethylphenyl)-3-(trifluoromethyl)-4-pyridyl]oxymethyl]-3,3-dimethyl-butyl]carbamate (290 mg, 0.5852 mmol) in anhydrous tetrahydrofuran (11.5 mL) under nitrogen atmosphere was cooled down to 0° C. Then a solution of sodium tert-pentoxide 40% w/v in toluene (500 μL of 40% w/v, 1.8160 mmol) was added dropwise. The reaction was stirred at 0° C. for 30 minutes then at room temperature for 5 hours. The reaction was then cooled down to 0° C. and quenched by the addition of an aqueous solution of 1N hydrochloric acid (20 mL). The layers were separated and the aqueous layer was extracted with ethyl acetate (4×30 mL). The combined organic layers were washed with brine (100 mL), dried over magnesium sulfate, filtered, and concentrated under reduced pressure to afford an-off white powder. The crude was then dissolved in a 1/1 v/v mixture of tetrahydrofuran (5.5 mL) and water (5.5 mL) and lithium hydroxide hydrate (75 mg, 1.7873 mmol) was added. The reaction was stirred at room temperature for 2 hours. The reaction was then cooled down to 0° C. then quenched until pH=2-3 with an aqueous solution of 1N hydrochloric acid (10 mL). The layers were separated, and the aqueous layer was extracted with ethyl acetate (5×10 mL). The combined organic layers were washed with brine (25 mL), dried over magnesium sulfate, filtered and concentrated under reduced pressure to afford crude 3-[[4-[(2R)-2-(tert-butoxycarbonylamino)-4,4-dimethyl-pentoxy]-6-(2,6-dimethylphenyl)-5-(trifluoromethyl)-2-pyridyl]sulfamoyl]benzoic acid (364 mg, 80%) as beige foam which was directly used in the next Step without further purification. ¹H NMR (400 MHz, DMSO-d₆, 80 C) δ 12.30 (br. s, 1H), 8.33 (t, J=1.6 Hz, 1H), 8.11 (dt, J=7.6, 1.3 Hz, 1H), 8.04-7.95 (m, 1H), 7.55 (t, J=7.8 Hz, 1H), 7.21-7.14 (m, 1H), 7.08-7.01 (m, 2H), 6.85 (s, 1H), 6.44 (br. s, 1H), 4.02-3.91 (m, 3H), 1.79 (br. s., 6H), 1.42-1.34 (m, 11H), 0.93 (s, 9H). (1H missing, labile proton).19F NMR (377 MHz, DMSO-d₆, 80 C) δ −56.04 (s, 3F). ESI-MS m/z calc. 679.2539, found 680.2 (M+1)⁺; Retention time: 3.07 minutes; LC method 1D.

### Step 7: 3-[[4-[(2R)-2-Amino-4,4-dimethyl-pentoxy]-6-(2,6-dimethylphenyl)-5-(trifluoromethyl)-2-pyridyl]sulfamoyl]benzoic acid

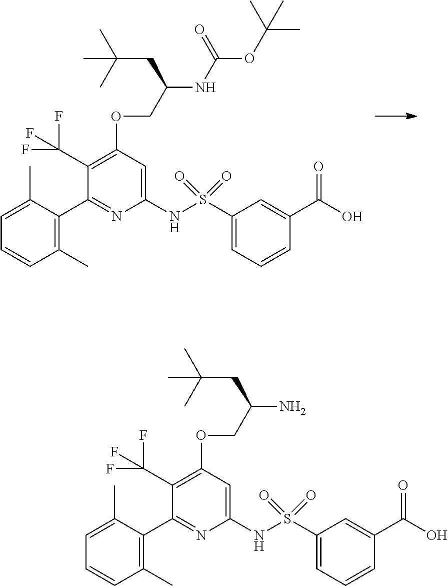 [SMILES: Cc1cccc(C)c1-c1nc(NS(=O)(=O)c2cccc(C(=O)O)c2)cc(OC[C@@H](CC(C)(C)C)NC(=O)OC(C)(C)C)c1C(F)(F)F ; Cc1cccc(C)c1-c1nc(NS(=O)(=O)c2cccc(C(=O)O)c2)cc(OC[C@H](N)CC(C)(C)C)c1C(F)(F)F]

To a stirred solution of 3-[[4-[(2R)-2-(tert-butoxycarbonylamino)-4,4-dimethyl-pentoxy]-6-(2,6-dimethylphenyl)-5-(trifluoromethyl)-2-pyridyl]sulfamoyl]benzoic acid (364 mg, 0.4686 mmol) in anhydrous dichloromethane (10 mL) under nitrogen atmosphere at 0° C. was added a solution of hydrogen chloride 4M in dioxane (2.7 mL of 4 M, 10.800 mmol). The reaction was stirred for 30 minutes at 0° C. then stirred overnight at room temperature. The reaction was then concentrated under reduced pressure then purified by reverse-phase chromatography on C₁₈ (column: 30 g HP Gold C₁₈, gradient: 30 to 100% methanol in water containing 0.1% v/v of hydrochloric acid, 15 CV). The desired fractions were concentrated under reduced pressure and the white residue was freeze-dried to afford 3-[[4-[(2R)-2-amino-4,4-dimethyl-pentoxy]-6-(2,6-dimethylphenyl)-5-(trifluoromethyl)-2-pyridyl]sulfamoyl]benzoic acid (hydrochloride salt) (179 mg, 62%) as a white fluffy powder. ¹H NMR (400 MHz, DMSO-d₆, 80 C) δ 8.35-8.32 (m, 1H), 8.18-8.08 (m, 1H), 8.04-7.95 (m, 1H), 7.57 (t, J=7.8 Hz, 1H), 7.27-7.15 (m, 1H), 7.12-6.98 (m, 2H), 6.89 (s, 1H), 4.36 (dd, J=10.0, 4.2 Hz, 1H), 4.30 (dd, J=9.8, 4.4 Hz, 1H), 3.67-3.60 (m, 1H), 1.86-1.71 (m, 7H), 1.59 (dd, J=14.7, 5.4 Hz, 1H), 0.97 (s, 9H). (5H missing, labile protons).19F NMR (377 MHz, DMSO-d₆, 80 C) δ −55.48 (s, 3F). ESI-MS m/z calc. 579.2015, found 580.2 (M+1)⁺; Retention time: 1.47 minutes; LC method X.

### Step 8: 3-[[4-[(2R)-2-[[6-[Cyclobutyl(methyl)amino]pyrazin-2-yl]methylamino]-4,4-dimethyl-pentoxy]-6-(2,6-dimethylphenyl)-5-(trifluoromethyl)-2-pyridyl]sulfamoyl]benzoic acid

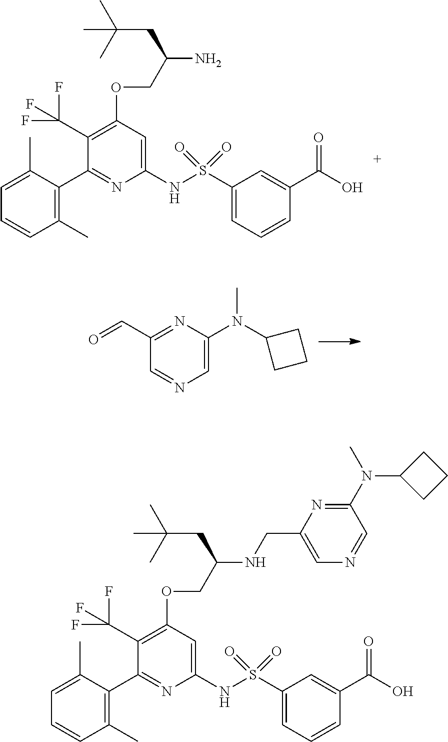 [SMILES: Cc1cccc(C)c1-c1nc(NS(=O)(=O)c2cccc(C(=O)O)c2)cc(OC[C@H](N)CC(C)(C)C)c1C(F)(F)F ; CN(c1cncc(C=O)n1)C1CCC1 ; Cc1cccc(C)c1-c1nc(NS(=O)(=O)c2cccc(C(=O)O)c2)cc(OC[C@@H](CC(C)(C)C)NCc2cncc(N(C)C3CCC3)n2)c1C(F)(F)F]

A stirred solution of 3-[[4-[(2R)-2-amino-4,4-dimethyl-pentoxy]-6-(2,6-dimethylphenyl)-5-(trifluoromethyl)-2-pyridyl]sulfamoyl]benzoic acid (hydrochloride salt) (90 mg, 0.1459 mmol) in anhydrous dichloromethane (6 mL) under nitrogen atmosphere was cooled down to 10-15° C. and 6-[cyclobutyl(methyl)amino]pyrazine-2-carbaldehyde (30 mg, 0.1498 mmol) was added. The reaction was stirred for 15 minutes at this temperature after which time sodium triacetoxyborohydride (93 mg, 0.4388 mmol) was added. The reaction was then stirred at 10-15° C. for 45 minutes then quenched by the addition of an aqueous solution of 1N hydrochloric acid (15 mL). The layers were separated and the aqueous layer was extracted with dichloromethane (3×10 mL) and 2-methyltetrahydrofuran (3×15 mL). The combined organic layers were washed with brine, dried over magnesium sulfate, filtered and concentrated under reduced pressure to afford a bright yellow oil which was co-evaporated with toluene (4×5 mL), then dried under high vacuum to afford crude 3-[[4-[(2R)-2-[[6-[cyclobutyl(methyl)amino]pyrazin-2-yl]methylamino]-4,4-dimethyl-pentoxy]-6-(2,6-dimethylphenyl)-5-(trifluoromethyl)-2-pyridyl]sulfamoyl]benzoic acid (hydrochloride salt) (140 mg, 91%) as a yellow powder which was used in the next Step without further purification ESI-MS m/z calc. 754.31244, found 755.2 (M+1)⁺; Retention time: 1.62 minutes; LC method X.

### Step 9: (11R)-12-[[6-[Cyclobutyl(methyl)amino]pyrazin-2-yl]methyl]-6-(2,6-dimethylphenyl)-11-(2,2-dimethylpropyl)-2,2-dioxo-7-(trifluoromethyl)-9-oxa-2λ⁶-thia-3,5,12-triazatricyclo[12.3.1.14,8]nonadeca-1(18),4(19),5,7,14,16-hexaen-13-one

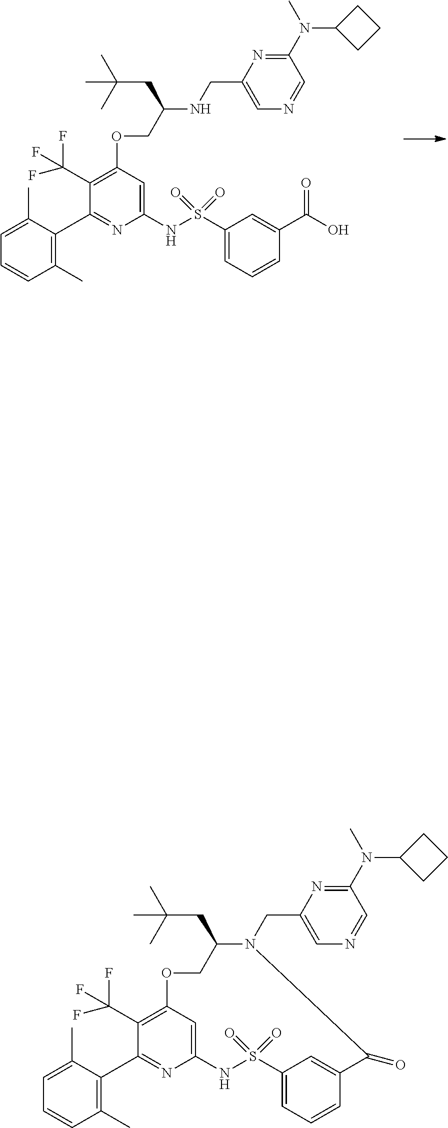 [SMILES: Cc1cccc(C)c1-c1nc(NS(=O)(=O)c2cccc(C(=O)O)c2)cc(OC[C@@H](CC(C)(C)C)NCc2cncc(N(C)C3CCC3)n2)c1C(F)(F)F ; Cc1cccc(C)c1-c1nc2cc(c1C(F)(F)F)OC[C@@H](CC(C)(C)C)N(Cc1cncc(N(C)C3CCC3)n1)C(=O)c1cccc(c1)S(=O)(=O)N2]

Crude 3-[[4-[(2R)-2-[[6-[cyclobutyl(methyl)amino]pyrazin-2-yl]methylamino]-4,4-dimethyl-pentoxy]-6-(2,6-dimethylphenyl)-5-(trifluoromethyl)-2-pyridyl]sulfamoyl] benzoic acid (hydrochloride salt) (140 mg, 0.1329 mmol) was dissolved in anhydrous DMF (17 mL) under nitrogen atmosphere and cooled down to 0° C. Then N-methyl morpholine (165.60 mg, 180 μL, 1.6372 mmol) was added and the mixture was stirred for 10 minutes after which time 2-chloro-4,6-dimethoxy-1,3,5-triazine (60 mg, 0.3417 mmol) was added. The reaction was stirred for 15 minutes at 0° C. then stirred at room temperature for 48h. The solvent was removed under reduced pressure and the crude yellow oil was purified by reverse-phase chromatography on Cis (column: 50 g HP Gold; gradient: 35 to 100% methanol in water containing 0.1% v/v of formic acid, 25 CV). The desired fractions were concentrated under reduced pressure and the residue was purified by reverse-phase chromatography on C₁₈ (column: 15.5 g HP Gold Cis; gradient: 35 to 100% methanol in 10 mM ammonium bicarbonate buffer pH=10; 25 CV). The volatiles were removed under reduced pressure and the white residue was freeze-dried to afford (11R)-12-[[6-[cyclobutyl(methyl)amino]pyrazin-2-yl]methyl]-6-(2,6-dimethylphenyl)-11-(2,2-dimethylpropyl)-2,2-dioxo-7-(trifluoromethyl)-9-oxa-2λ⁶-thia-3,5,12-triazatricyclo [12.3.1.14,8]nonadeca-1(18),4(19),5,7,14,16-hexaen-13-one (22 mg, 22%) as a white solid ¹H NMR (400 MHz, DMSO-d₆) δ 13.14 (br. s, 1H), 8.26 (br. s, 1H), 8.02 (s, 1H), 8.00-7.94 (m, 2H), 7.83-7.76 (m, 1H), 7.75-7.69 (m, 1H), 7.26-7.19 (m, 1H), 7.14-7.09 (m, 2H), 7.09-7.02 (m, 1H), 5.17 (dd, J=12.2, 2.9 Hz, 1H), 4.83 (quin, J=8.5 Hz, 1H), 4.72-4.58 (m, 2H), 4.49 (d, J=15.7 Hz, 1H), 3.83-3.71 (m, 1H), 3.11 (s, 3H), 2.27-2.17 (m, 4H), 1.99 (s, 3H), 1.85 (dd, J=15.3, 10.1 Hz, 1H), 1.74 (s, 3H), 1.71-1.64 (m, 2H), 1.42 (d, J=15.7 Hz, 1H), 0.57 (s, 9H). 19F NMR (377 MHz, DMSO-d₆) δ −55.54 (br. s, 3F). ESI-MS m/z calc. 736.3019, found 737.3 (M+1)⁺; Retention time: 3.31 minutes; LC method 1D.

### Preparation of Compound 334

### Step 1: 3-Methyl-2-(4,4,5,5-tetramethyl-1,3,2-dioxaborolan-2-yl)benzaldehyde

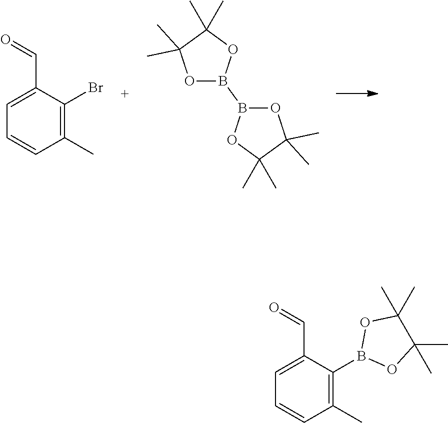

Prepared a solution of 2-bromo-3-methyl-benzaldehyde (22.5 g, 113.04 mmol), bis(pinacolato)diboron (43.1 g, 169.73 mmol), and KOAc (22.2 g, 226.20 mmol) in 1,4-dioxane (500 mL). The resulting slurry was sparged with a nitrogen stream for five minutes, then [1,1′-Bis(diphenylphosphino)ferrocene]dichloropalladium(II) (8.3 g, 11.343 mmol) was added and the mixture was refluxed under nitrogen for twenty hours, then cooled to room temperature and quenched with 1M hydrochloric acid until the pH was approximately 3-4. The phases were then separated: the aqueous phase was discarded and the organic phase was concentrated in vacuo, combined with a crude product from a previous batch run using 2.5 g of 2-bromo-3-methyl-benzaldehyde and purified by silica gel chromatography using 0 to 10% ethyl acetate in hexane to obtain 3-methyl-2-(4,4,5,5-tetramethyl-1,3,2-dioxaborolan-2-yl)benzaldehyde (22.5 g, 81%) as a pale yellow oil. ¹H NMR (500 MHz, Chloroform-d) δ 9.98 (s, 1H), 7.63 (dd, J=6.6, 2.1 Hz, 1H), 7.43 (d, J=6.6 Hz, 2H), 2.49 (s, 3H), 1.49 (s, 12H). ESI-MS m/z calc. 246.14273, found 247.2 (M+1)⁺; Retention time: 0.66 minutes; LC method S.

### Step 2: tert-Butyl N-tert-butoxycarbonyl-N-[4-chloro-6-(2-formyl-6-methyl-phenyl)-5-methyl-pyrimidin-2-yl]carbamate

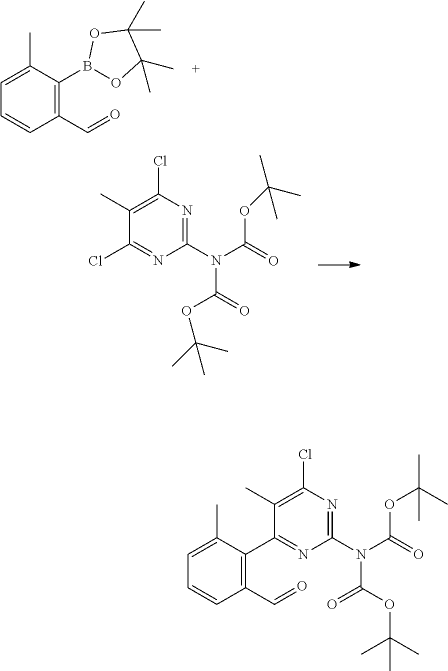

A flask was charged with 3-methyl-2-(4,4,5,5-tetramethyl-1,3,2-dioxaborolan-2-yl)benzaldehyde (19.2 g, 54.610 mmol), tert-butyl N-tert-butoxycarbonyl-N-(4,6-dichloro-5-methyl-pyrimidin-2-yl)carbamate (20 g, 49.702 mmol), cesium carbonate (46 g, 141.18 mmol) and Pd(dppf)Cl₂·DCM (4.06 g, 4.9716 mmol) under argon. A pre-degassed (bubbling argon for 1 h, both the solvents) dimethoxyethane (130 mL) and water (42 mL) were added to the reaction mixture. The reaction mixture was then further degassed with argon for 10 minutes. The mixture was then heated to 85° C. for 2 h. The reaction was then allowed to cool to RT and diluted with water (200 mL) and EtOAc (200 mL). The aqueous layer was extracted with EtOAc (3×200 mL). The organic layers were then combined, washed with brine (200 ml), dried with anhydrous sodium sulfate, filtered, and concentrated under reduced pressure. The crude product was purified by flash chromatography (Combiflash, loaded onto dry silica in 2×330 g cartridge using 0-40% diethyl ether in hexanes (1% Et₃N as modifier) as eluent) and the solvent was removed to provide tert-butyl N-tert-butoxycarbonyl-N-[4-chloro-6-(2-formyl-6-methyl-phenyl)-5-methyl-pyrimidin-2-yl]carbamate (9.2 g, 38%) as an off white solid. ¹H NMR (500 MHz, DMSO-d₆) δ 9.79 (s, 1H), 7.94 (dd, J=7.5, 1.4 Hz, 1H), 7.76-7.66 (m, 2H), 2.02 (s, 3H), 2.00 (s, 3H), 1.36 (s, 18H). ESI-MS m/z calc. 461.17175, found 462.1 (M+1)⁺; Retention time: 3.15 minutes; LC method W.

### Step 3: tert-Butyl N-tert-butoxycarbonyl-N-[4-chloro-6-[2-(hydroxymethyl)-6-methyl-phenyl]-5-methyl-pyrimidin-2-yl]carbamate

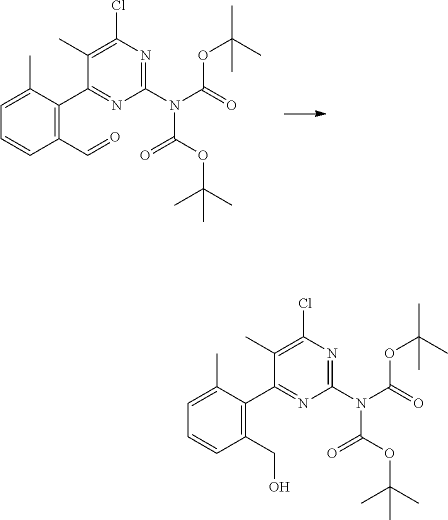 [SMILES: Cc1cccc(C=O)c1-c1nc(N(C(=O)OC(C)(C)C)C(=O)OC(C)(C)C)nc(Cl)c1C ; Cc1cccc(CO)c1-c1nc(N(C(=O)OC(C)(C)C)C(=O)OC(C)(C)C)nc(Cl)c1C]

tert-Butyl N-tert-butoxycarbonyl-N-[4-chloro-6-(2-formyl-6-methyl-phenyl)-5-methyl-pyrimidin-2-yl]carbamate (3 g, 5.8449 mmol) was dissolved in anhydrous THF (28.5 mL) and the solution was cooled to 0° C. Sodium borohydride (425 mg, 0.4497 mL, 11.234 mmol) was then added portionwise to it. The reaction was then stirred for 30 min at 0° C. The reaction was then warmed up to room temperature and quenched with water (50 ml) and saturated ammonium chloride solution (50 ml) and EtOAc (100 ml) was added to it. The reaction mixture was then stirred at room temperature for 10 min. The aqueous layer was extracted with EtOAc (3×100 ml) and the combined organic solution was washed with brine (70 ml), dried over anhydrous sodium sulfate, filtered and the solvent was removed under reduced pressure. The crude product was purified by flash chromatography (Combiflash, loaded onto dry silica in 80 g cartridge using 0-50% EtOAc in hexanes (1% Et₃N as modifier) as eluent to provide tert-butyl N-tert-butoxycarbonyl-N-[4-chloro-6-[2-(hydroxymethyl)-6-methyl-phenyl]-5-methyl-pyrimidin-2-yl]carbamate (2.51 g, 87%) as a colorless foamy solid. ESI-MS m/z calc. 463.1874, found 464.3 (M+1)⁺; Retention time: 6.15 minutes; LC method S.

### Step 4: tert-Butyl N-[4-[2-(bromomethyl)-6-methyl-phenyl]-6-chloro-5-methyl-pyrimidin-2-yl]-N-tert-butoxycarbonyl-carbamate

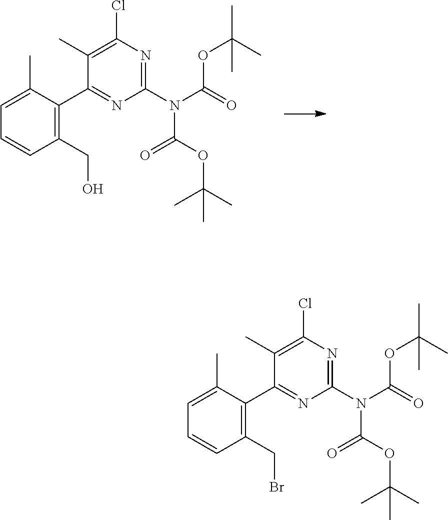 [SMILES: Cc1cccc(CO)c1-c1nc(N(C(=O)OC(C)(C)C)C(=O)OC(C)(C)C)nc(Cl)c1C ; Cc1cccc(CBr)c1-c1nc(N(C(=O)OC(C)(C)C)C(=O)OC(C)(C)C)nc(Cl)c1C]

tert-Butyl N-tert-butoxycarbonyl-N-[4-chloro-6-[2-(hydroxymethyl)-6-methyl-phenyl]-5-methyl-pyrimidin-2-yl]carbamate (7.5 g, 15.034 mmol) and carbon tetrabromide (7.5 g, 22.616 mmol) were dissolved in anhydrous dichloromethane (75 mL) and the solution was cooled to 0° C. A solution of triphenyl phosphine (6 g, 22.876 mmol) in anhydrous dichloromethane (23 mL) was added dropwise over 10 min. The reaction mixture was then stirred at the same temperature for 45 min (NOTE). The solvent was removed by rotary evaporation. The crude product was purified by flash chromatography (Combiflash, loaded onto dry silica in 220 g cartridge using 0-30% diethyl ether in hexanes (1% Et₃N as modifier) as eluent and the solvent was removed to provide tert-butyl N-[4-[2-(bromomethyl)-6-methyl-phenyl]-6-chloro-5-methyl-pyrimidin-2-yl]-N-tert-butoxycarbonyl-carbamate (7.3 g, 83%) as white foamy solid. ESI-MS m/z calc. 525.10297, found 526.5 (M+1)⁺; Retention time: 7.88 minutes; LC method S.

### Step 5: [2-[2-[bis(tert-Butoxycarbonyl)amino]-6-chloro-5-methyl-pyrimidin-4-yl]-3-methyl-phenyl]methyl-triphenyl-phosphonium

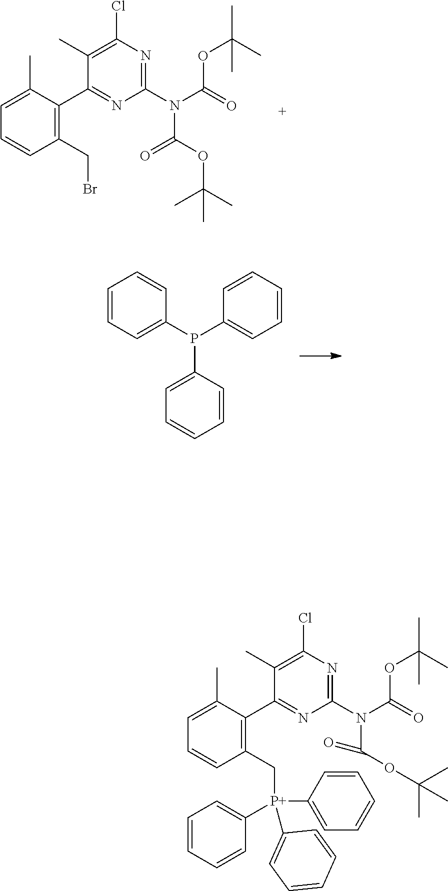 [SMILES: Cc1cccc(CBr)c1-c1nc(N(C(=O)OC(C)(C)C)C(=O)OC(C)(C)C)nc(Cl)c1C ; c1ccc(P(c2ccccc2)c2ccccc2)cc1 ; Cc1cccc(C[P+](c2ccccc2)(c2ccccc2)c2ccccc2)c1-c1nc(N(C(=O)OC(C)(C)C)C(=O)OC(C)(C)C)nc(Cl)c1C]

tert-Butyl N-[4-[2-(bromomethyl)-6-methyl-phenyl]-6-chloro-5-methyl-pyrimidin-2-yl]-N-tert-butoxycarbonyl-carbamate (3.15 g, 5.3810 mmol) was dissolved in anhydrous toluene (22 mL) at room temperature and the solution was degassed (bubbling argon) for 5 min. triphenylphosphane (1.6 g, 1.4134 mL, 6.1002 mmol) was then added to the solution and stirred for 5 min at room temperature under nitrogen. The reaction mixture was then heated at 85° C. for 5 h (white solid started crashing out after 30 min of heating which was indicated the formation of phosphonium salt) and the starting material was completely consumed at this time. The reaction mixture was then cooled to room temperature and white solid formed was then filtered through sintered funnel washing thoroughly with cold toluene (cooling in ice-water bath) to remove most of the organic impurities (excess triphenylphosphine, triphenylphosphine oxide) and the solid was dried under vacuum to give [2-[2-[bis(tert-butoxycarbonyl)amino]-6-chloro-5-methyl-pyrimidin-4-yl]-3-methyl-phenyl]methyl-triphenyl-phosphonium (Bromide Ion (1)) (3.8 g, 85%) as white solid. ¹H NMR (500 MHz, DMSO-d₆) δ 7.90-7.83 (m, 3H), 7.70-7.62 (m, 6H), 7.55-7.43 (m, 6H), 7.39-7.33 (m, 1H), 7.23 (t, J=7.8 Hz, 1H), 6.78 (dd, J=7.9, 2.4 Hz, 1H), 4.99 (t, J=15.4 Hz, 1H), 4.48 (t, J=15.7 Hz, 1H), 1.88 (s, 3H), 1.76 (s, 3H), 1.39 (s, 18H). ESI-MS m/z calc. 708.2758, found 708.7 (M+)⁺; null (M−)⁺; Retention time: 3.02 minutes; LC method W.

### Step 6: tert-Butyl N-tert-butoxycarbonyl-N-[4-chloro-5-methyl-6-[2-methyl-6-[(E)-3-methylbut-1-enyl]phenyl]pyrimidin-2-yl]carbamate;tert-butyl N-tert-butoxycarbonyl-N-[4-chloro-5-methyl-6-[2-methyl-6-[(Z)-3-methylbut-1-enyl]phenyl]py

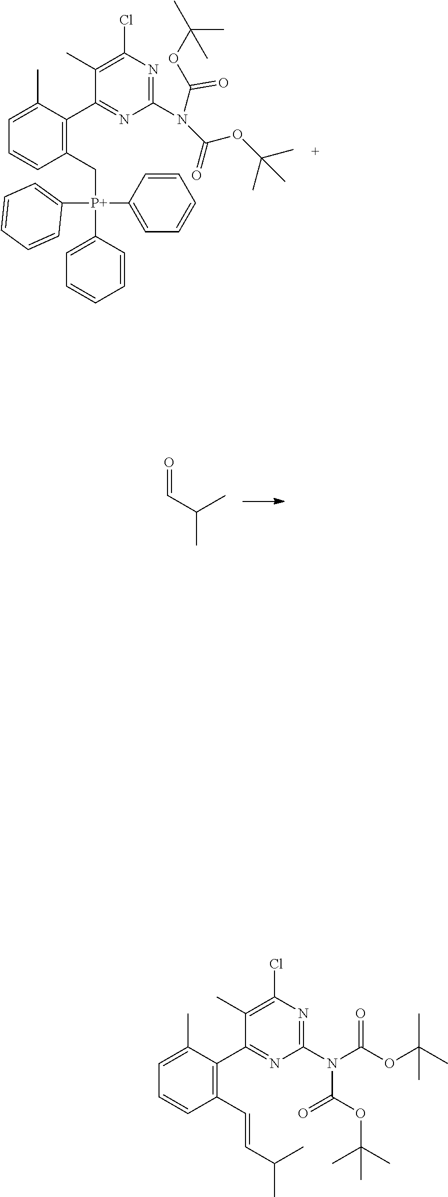

To a solution of [2-[2-[bis(tert-butoxycarbonyl)amino]-6-chloro-5-methyl-pyrimidin-4-yl]-3-methyl-phenyl]methyl-triphenyl-phosphonium (Bromide Ion (1)) (5 g, 6.3360 mmol) in DCM (20 mL) was added potassium carbonate (880 mg, 6.3673 mmol) and 18-Crown-6 (335 mg, 1.2674 mmol) at room temperature and the resulting orange solution was added 2-methylpropanal (922.50 mg, 0.750 mL, 12.794 mmol). The mixture was heated in a 450 C oil bath for 3.5 h. The reaction was stirred at RT 72 h. The reaction was concentrated in vacuo, triturated in TBME (50 mL) at 550 C for 30 min cooled to room temperature and filtered through packed celite, then rinsed filter with TBME (2×50 mL). The filtrate was concentrated in vacuo and the resulting residue was purified by flash chromatography (Combiflash, loaded onto [pre-equilibrated with hexanes-Et₃N (0.5%)] 220 g SiO₂ cartridge with benzene, and eluted with 0-4% EtOAc in Hexanes over 90 minute gradient; no Et₃N used during elution) to provide tert-butyl N-tert-butoxycarbonyl-N-[4-chloro-5-methyl-6-[2-methyl-6-[(E)-3-methylbut-1-enyl]phenyl]pyrimidin-2-yl]carbamate;tert-butyl N-tert-butoxycarbonyl-N-[4-chloro-5-methyl-6-[2-methyl-6-[(Z)-3-methylbut-1-enyl]phenyl]py (2.5 g, 75%) as a colorless oil (major product E-isomer). ¹H NMR (500 MHz, Chloroform-d) δ 7.44-7.40 (m, 1H), 7.32-7.27 (m, 1H), 7.17-7.13 (m, 1H), 6.05 (dd, J=15.6, 7.5 Hz, 1H), 5.81 (d, J=15.5 Hz, 1H), 2.31-2.20 (m, 1H), 2.08 (s, 3H), 2.01 (s, 3H), 1.44 (s, 18H), 0.96 (dd, J=6.7, 2.3 Hz, 6H). ESI-MS m/z calc. 501.23944, found 502.5 (M+1)⁺; Retention time: 8.78 minutes; LC method S.

### Step 7: tert-Butyl N-tert-butoxycarbonyl-N-[4-chloro-6-(2-isopentyl-6-methyl-phenyl)-5-methyl-pyrimidin-2-yl]carbamate

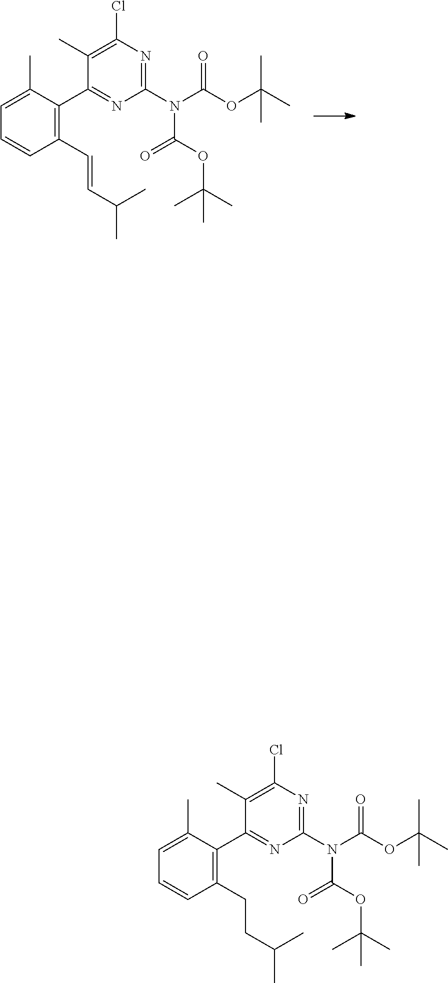 [SMILES: Cc1cccc(/C=C/C(C)C)c1-c1nc(N(C(=O)OC(C)(C)C)C(=O)OC(C)(C)C)nc(Cl)c1C ; Cc1cccc(CCC(C)C)c1-c1nc(N(C(=O)OC(C)(C)C)C(=O)OC(C)(C)C)nc(Cl)c1C]

To a solution of tert-butyl N-tert-butoxycarbonyl-N-[4-chloro-5-methyl-6-[2-methyl-6-[(E)-3-methylbut-1-enyl]phenyl]pyrimidin-2-yl]carbamate (2.5 g, 4.9796 mmol) in EtOAc (200 mL) was added Pd-C (475 mg, 10% w/w, 0.4463 mmol) under N₂. The reaction was cooled to 0° C. then bubbled H₂ gas through the reaction then placed under a H₂ balloon and stirred at room temperature. The reaction was stirred 4 h at 0° C. The reaction was purged with N₂ gas then filtered through packed celite and the filtrate concentrated in vacuo to provide tert-butyl N-tert-butoxycarbonyl-N-[4-chloro-6-(2-isopentyl-6-methyl-phenyl)-5-methyl-pyrimidin-2-yl]carbamate (2.48 g, 98%). ¹H NMR (500 MHz, Chloroform-d) δ 7.31-7.27 (m, 1H), 7.14 (dd, J=19.6, 7.6 Hz, 2H), 2.31-2.22 (m, 2H), 2.12 (s, 3H), 1.97 (s, 3H), 1.43 (s, 18H), 1.31-1.22 (m, 2H), 0.98-0.93 (m, 1H), 0.81 (dd, J=6.4, 1.3 Hz, 6H). ESI-MS m/z calc. 503.2551, found 504.5 (M+1)⁺; Retention time: 8.82 minutes; LC method S.

### Step 8: 4-Chloro-6-(2-isopentyl-6-methyl-phenyl)-5-methyl-pyrimidin-2-amine

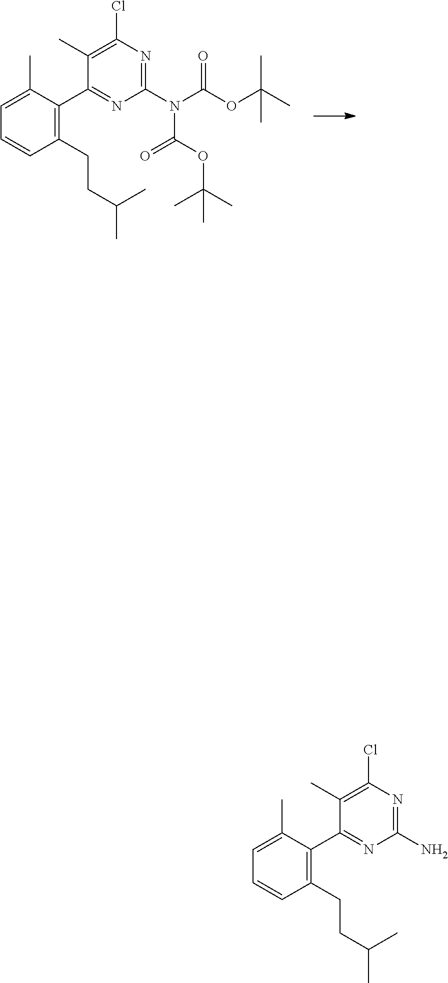 [SMILES: Cc1cccc(CCC(C)C)c1-c1nc(N(C(=O)OC(C)(C)C)C(=O)OC(C)(C)C)nc(Cl)c1C ; Cc1cccc(CCC(C)C)c1-c1nc(N)nc(Cl)c1C]

A solution of tert-butyl N-tert-butoxycarbonyl-N-[4-chloro-6-(2-isopentyl-6-methyl-phenyl)-5-methyl-pyrimidin-2-yl]carbamate (2.48 g, 4.9200 mmol) in HFIP (40 mL) split half evenly dispensed over 2 microwave vials and each was heated at 100° C. in a microwave reactor for 1 h. The contents of both vials were combined and the reaction was concentrated in vacuo. The resulting residue was purified by flash chromatography (Combiflash, loaded onto an 80 g SiO₂ cartridge with benzene, and eluted with 0-5% EtOAc in Hexanes over a 45 minute gradient) to provide 4-chloro-6-(2-isopentyl-6-methyl-phenyl)-5-methyl-pyrimidin-2-amine (1.0659 g, 70%) (yield is over 2-steps) as a colorless oil. ¹H NMR (500 MHz, Chloroform-d) δ 7.23 (t, J=7.7 Hz, 1H), 7.14-7.06 (m, 2H), 5.13-5.01 (m, 2H), 2.34 (ddd, J=13.6, 10.6, 5.6 Hz, 1H), 2.22 (ddd, J=13.6, 10.9, 5.2 Hz, 1H), 2.03 (s, 3H), 1.93 (d, J=0.5 Hz, 3H), 1.48-1.35 (m, 2H), 1.33-1.24 (m, 1H), 0.84-0.73 (m, 6H). ESI-MS m/z calc. 303.15024, found 304.0 (M+1)⁺; Retention time: 2.99 minutes; LC method W.

### Step 9: Methyl 3-[[4-chloro-6-(2-isopentyl-6-methyl-phenyl)-5-methyl-pyrimidin-2-yl]sulfamoyl]benzoate

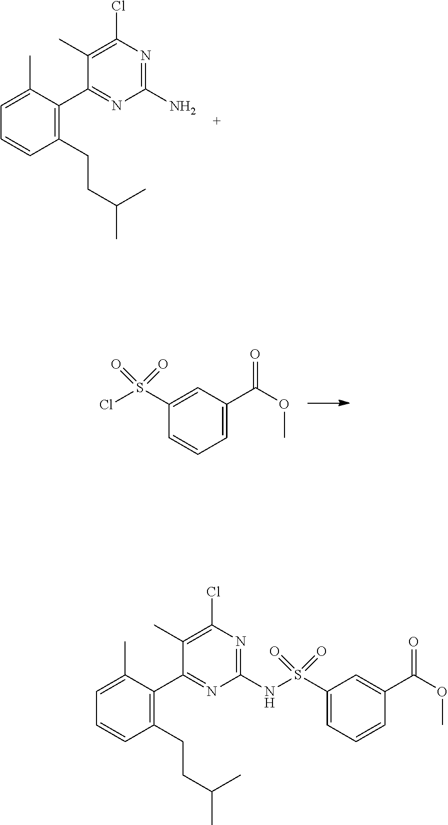

To a solution of 4-chloro-6-(2-isopentyl-6-methyl-phenyl)-5-methyl-pyrimidin-2-amine (768 mg, 2.4671 mmol) and methyl 3-chlorosulfonylbenzoate (1.451 g, 6.1835 mmol) in anhydrous THF (41 mL) at −78° C. under nitrogen was added LiHMDS in THF (7.6 mL of 1.3 M, 9.8800 mmol) dropwise. The mixture was stirred at −78° C. for 2 h. The reaction was quenched still cold with 2 N HCl aqueous solution (23 mL) and EtOAc (20 mL). the mixture was allowed to warm up to 0° C. and stirred for 10 minutes before letting it warm to room temperature. The layers were separated and the aqueous layer was extracted with EtOAc (3×20 mL). The combined organic layers were washed with brine, dried over anhydrous sodium sulfate and concentrated under reduced pressure. The crude product was purified by silica gel column chromatography (0-20% EtOAc in Hexanes) to afford methyl 3-[[4-chloro-6-(2-isopentyl-6-methyl-phenyl)-5-methyl-pyrimidin-2-yl]sulfamoyl]benzoate (1.067 g, 85%) as a colorless oil. ESI-MS m/z calc. 501.1489, found 502.1 (M+1)⁺; Retention time: 3.99 minutes; LC method T.

### Step 10: 3-[[4-Chloro-6-(2-isopentyl-6-methyl-phenyl)-5-methyl-pyrimidin-2-yl]sulfamoyl]benzoic acid

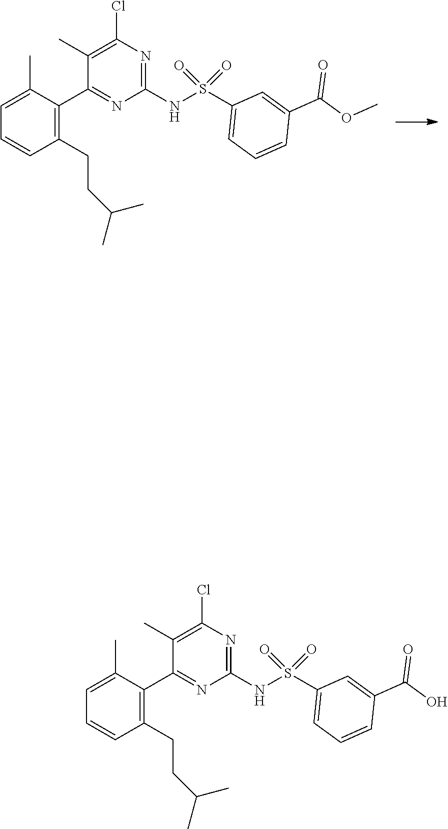 [SMILES: COC(=O)c1cccc(S(=O)(=O)Nc2nc(Cl)c(C)c(-c3c(C)cccc3CCC(C)C)n2)c1 ; Cc1cccc(CCC(C)C)c1-c1nc(NS(=O)(=O)c2cccc(C(=O)O)c2)nc(Cl)c1C]

To a solution of methyl 3-[[4-chloro-6-(2-isopentyl-6-methyl-phenyl)-5-methyl-pyrimidin-2-yl]sulfamoyl]benzoate (1.067 g, 2.0829 mmol) in THF (20 mL) was added NaOH (17 mL of 1 M, 17.000 mmol) and stirred at room temperature for 3 h. After completion, 2M HCl (12 mL) was added to acidify the solution. The two layers were separated and the aqueous layer was extracted with DCM (3×15 mL), the combined organic layers were dried over anhydrous sodium sulfate and concentrated under reduced pressure to afford 3-[[4-chloro-6-(2-isopentyl-6-methyl-phenyl)-5-methyl-pyrimidin-2-yl]sulfamoyl]benzoic acid (1.051 g, 99%) as a white solid. ¹H NMR (500 MHz, DMSO-d₆) δ 13.35 (s, 1H), 12.20 (s, 1H), 8.43-8.36 (m, 1H), 8.19-8.01 (m, 2H), 7.68-7.56 (m, 1H), 7.33-7.16 (m, 1H), 7.16-7.03 (m, 2H), 2.07-1.98 (m, 1H), 1.91-1.86 (m, 1H), 1.84 (s, 3H), 1.74 (s, 3H), 1.20-1.13 (m, 1H), 1.11-1.03 (m, 1H), 1.01-0.94 (m, 1H), 0.60-0.42 (m, 6H). ESI-MS m/z calc. 487.13324, found 488.2 (M+1)⁺; Retention time: 2.9 minutes; LC method W.

### Step 11: 3-[[4-[(2R)-2-amino-4,4-dimethyl-pentoxy]-6-(2-isopentyl-6-methyl-phenyl)-5-methyl-pyrimidin-2-yl]sulfamoyl]benzoic acid

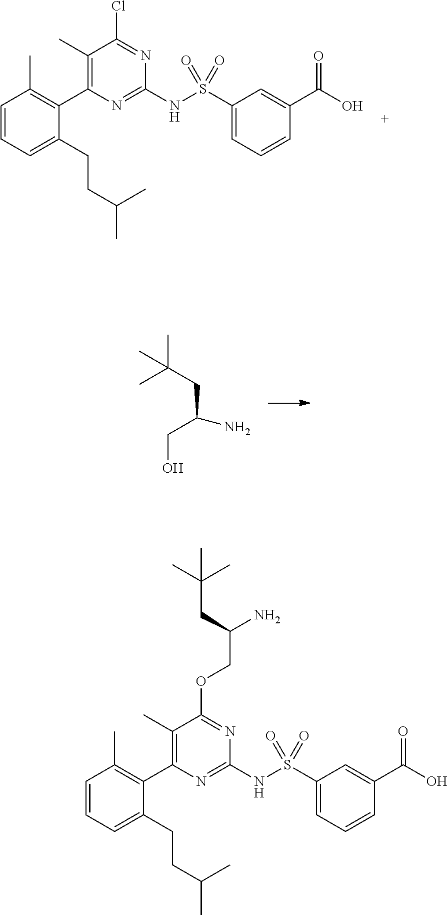

To a vigorously stirring, pre-sonicated suspension of 3-[[4-chloro-6-(2-isopentyl-6-methyl-phenyl)-5-methyl-pyrimidin-2-yl]sulfamoyl]benzoic acid (511 mg, 1.0367 mmol) and (2R)-2-amino-4,4-dimethyl-pentan-1-ol (hydrochloride salt) (527 mg, 3.1430 mmol) in anhydrous THF (10 mL) at 0° C. under nitrogen was portionwise added sodium tert-butoxide (945 mg, 9.8332 mmol). The reaction mixture was allowed to warm up to room temperature and stirred for 4 hours. The reaction mixture was cooled to 0° C., and slowly quenched with 2 M aqueous HCl (9 mL). The reaction mixture was allowed to warm up to room temperature and stirred for 10 minutes. Ethyl acetate (10 mL) was added, and the reaction mixture was vigorously stirred for 10 minutes. The two layers were separated, and the aqueous layer was extracted with ethyl acetate (3×15 mL). Combined organic layers were washed with brine, dried over anhydrous sodium sulfate and concentrated to afford 3-[[4-[(2R)-2-amino-4,4-dimethyl-pentoxy]-6-(2-isopentyl-6-methyl-phenyl)-5-methyl-pyrimidin-2-yl]sulfamoyl]benzoic acid (hydrochloride salt) (547 mg, 77%) as an off white solid. ¹H NMR (500 MHz, DMSO-d₆) δ 8.51-8.39 (m, 1H), 8.18-7.99 (m, 2H), 7.72-7.54 (m, 1H), 7.38-7.24 (m, 1H), 7.21-7.01 (m, 2H), 4.46-4.30 (m, 1H), 4.19 (d, J=5.2 Hz, 1H), 4.10-3.93 (m, 1H), 3.67-3.50 (m, 1H), 2.30-2.13 (m, 1H), 2.13-2.00 (m, 1H), 1.99-1.84 (m, 3H), 1.73-1.65 (m, 3H), 1.64-1.45 (m, 2H), 1.37-1.03 (m, 2H), 0.93 (s, 4H), 0.90 (s, 5H), 0.69-0.49 (m, 6H). ESI-MS m/z calc. 582.2876, found 583.3 (M+1)⁺; Retention time: 2.14 minutes; LC method W.

### Step 12: 3-{[(1M)-4-{[(2R)-4,4-Dimethyl-2-({[5-(propan-2-yloxy)pyrimidin-2-yl]methyl}amino)pentyl]oxy}-5-methyl-6-[2-methyl-6-(3-methylbutyl)phenyl]pyrimidin-2-yl]sulfamoyl}benzoic acid and 3-{[(1P)-4-{[(2R)-4,4-dimethyl-2-({[5-(propan-2-yloxy)pyrimidin-2-yl]methyl}amino)pentyl]oxy}-5-methyl-6-[2-methyl-6-(3-methylbutyl)phenyl]pyrimidin-2-yl]sulfamoyl}benzoic acid

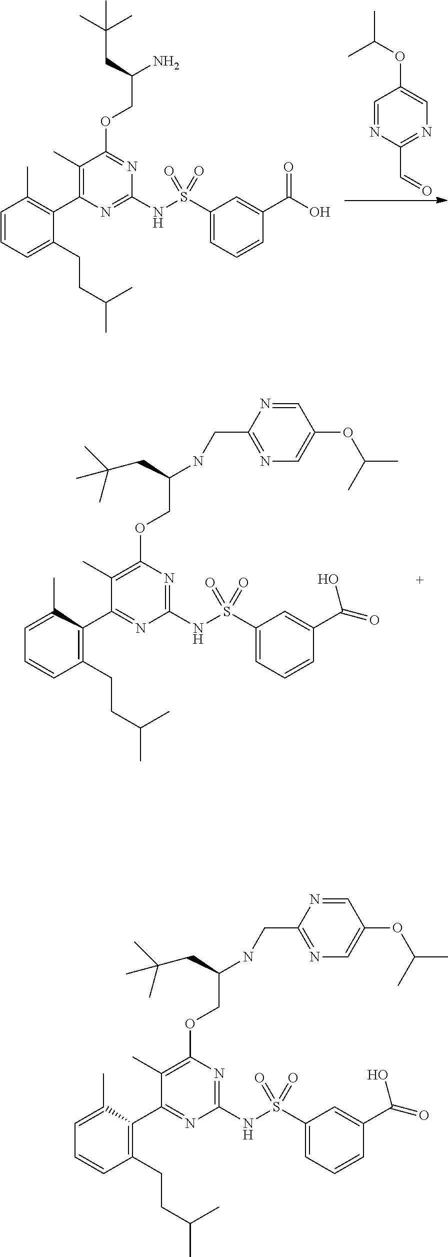

A 4 mL vial was charged under nitrogen with 3-[[4-[(2R)-2-amino-4,4-dimethyl-pentoxy]-6-(2-isopentyl-6-methyl-phenyl)-5-methyl-pyrimidin-2-yl]sulfamoyl]benzoic acid (hydrochloride salt) (100 mg, 0.1615 mmol), 5-isopropoxypyrimidine-2-carbaldehyde (28 mg, 0.1685 mmol) and DCM (450 μL). The mixture was stirred at room temperature for 15 min. sodium triacetoxyborohydride (46 mg, 0.2170 mmol) was added and the mixture was stirred at room temperature for 45 min. More sodium triacetoxyborohydride (46 mg, 0.2170 mmol) was added and the mixture was stirred at room temperature for an additional 30 min. The reaction was quenched with a few drops of 1N aqueous HCl. Methanol and DMSO were added (4 mL total volume). After filtration, purification by reverse phase HPLC (1-99% acetonitrile/5 mM aqueous HCl over 30 min) provided two isomers as tan solids: First to elute, more polar isomer, 3-{[(1M)-4-{[(2R)-4,4-dimethyl-2-({[5-(propan-2-yloxy)pyrimidin-2-yl]methyl}amino) pentyl]oxy}-5-methyl-6-[2-methyl-6-(3-methylbutyl)phenyl]pyrimidin-2-yl]sulfamoyl}benzoic acid (hydrochloride salt) (36.5 mg, 59%), ESI-MS m/z calc. 732.3669, found 733.69 (M+1)⁺; Retention time: 1.61 minutes (contains 17% of second isomer), and a second to elute, less polar isomer: 3-{[(1P)-4-{[(2R)-4,4-dimethyl-2-({[5-(propan-2-yloxy)pyrimidin-2-yl]methyl}amino)pentyl]oxy}-5-methyl-6-[2-methyl-6-(3-methylbutyl)phenyl]pyrimidin-2-yl]sulfamoyl}benzoic acid (hydrochloride salt) (26 mg, 42%), ESI-MS m/z calc. 732.3669, found 733.69 (M+1)⁺; Retention time: 1.64 minutes; LC method A.

### Step 13: (5P,11R)-11-(2,2-Dimethylpropyl)-7-methyl-6-[2-methyl-6-(3-methylbutyl)phenyl]-12-{[5-(propan-2-yloxy)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione

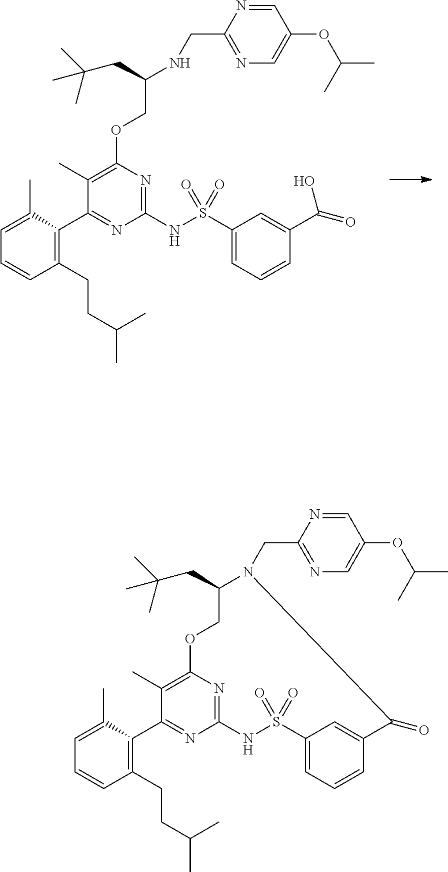

3-{[(1P)-4-{[(2R)-4,4-dimethyl-2-({[5-(propan-2-yloxy)pyrimidin-2-yl]methyl}amino)pentyl]oxy}-5-methyl-6-[2-methyl-6-(3-methylbutyl)phenyl]pyrimidin-2-yl]sulfamoyl}benzoic acid (hydrochloride salt) (26 mg, 0.03379 mmol) was combined in a 4 mL vial under nitrogen with CDMT (8.898 mg, 0.05068 mmol) and DMF (1 mL). The solution was stirred at 0° C. 4-methylmorpholine (19 μL, 0.1728 mmol) was added and the mixture was stirred in the cooling bath that was allowed to warm to room temperature overnight. After 17 h, the reaction was diluted with DMSO and filtered. Purification by reverse phase HPLC (1-99% acetonitrile/5 mM aqueous HCl over 30 min) gave (5P,11R)-11-(2,2-dimethylpropyl)-7-methyl-6-[2-methyl-6-(3-methylbutyl)phenyl]-12-{[5-(propan-2-yloxy)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (11.0 mg, 45%) as an off-white solid. ¹H NMR (400 MHz, DMSO-d₆) δ 13.35-11.54 (broad m, 1H), 8.70 (br s, 1H), 8.52 (s, 2H), 7.94 (br s, 1H), 7.66 (br s, 2H), 7.30 (s, 1H), 7.15 (d, J=25.5 Hz, 2H), 5.42 (s, 1H), 4.87 (d, J=16.5 Hz, 1H), 4.81 (p, J=6.0 Hz, 1H), 4.65 (d, J=16.5 Hz, 1H), 4.37-4.13 (m, 1H), 4.13-3.96 (m, 1H), 2.39-2.15 (m, 2H), 1.95-1.69 (m, 4H), 1.61 (s, 3H), 1.49-1.19 (m, 10H), 0.79 (s, 6H), 0.54 (s, 9H). ESI-MS m/z calc. 714.3563, found 715.7 (M+1)⁺; Retention time: 2.26 minutes; LC method A.

### Preparation of Compound 333

### Step 1: (5M,11R)-11-(2,2-Dimethylpropyl)-7-methyl-6-[2-methyl-6-(3-methylbutyl)phenyl]-12-{[5-(propan-2-yloxy)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione

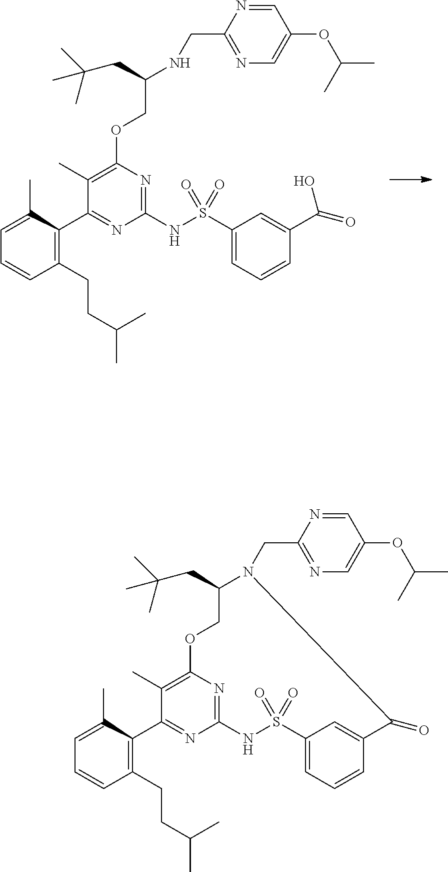

3-{[(1M)-4-{[(2R)-4,4-dimethyl-2-({[5-(propan-2-yloxy)pyrimidin-2-yl]methyl}amino)pentyl]oxy}-5-methyl-6-[2-methyl-6-(3-methylbutyl)phenyl]pyrimidin-2-yl]sulfamoyl}benzoic acid (hydrochloride salt) (36.5 mg, 0.04744 mmol) was combined in a 4 mL vial under nitrogen with CDMT (17 mg, 0.09683 mmol) and DMF (1 mL). The solution was stirred at 0° C. 4-Methyl-morpholine (26 μL, 0.2365 mmol) was added and the mixture was stirred in the cooling bath that was allowed to warm to room temperature overnight. After 17 h, the reaction was diluted with DMSO and filtered. Purification by reverse phase HPLC (1-99% acetonitrile/5 mM aqueous HCl over 30 min) gave (5M,11R)-11-(2,2-dimethylpropyl)-7-methyl-6-[2-methyl-6-(3-methylbutyl)phenyl]-12-{[5-(propan-2-yloxy)pyrimidin-2-yl]methyl}-9-oxa-2λ⁶-thia-3,5,12,19-tetraazatricyclo[12.3.1.14,8] nonadeca-1(17),4(19),5,7,14(18),15-hexaene-2,2,13-trione (13.7 mg, 40%) as an off-white solid. ¹H NMR (400 MHz, DMSO-d₆) δ 13.34-11.61 (broad m, 1H), 8.68 (br s, 1H), 8.52 (s, 2H), 7.86 (br s, 1H), 7.62 (br s, 2H), 7.30 (br s, 1H), 7.22-7.01 (m, 2H), 5.45 (br s, 1H), 4.94-4.75 (m, 2H), 4.66 (d, J=16.5 Hz, 1H), 4.19 (t, J=11.3 Hz, 1H), 3.92 (td, J=10.5, 9.8, 4.2 Hz, 1H), 2.16 (s, 1H), 2.04 (s, 3H), 1.86 (dd, J=15.2, 9.6 Hz, 2H), 1.58 (s, 3H), 1.37 (d, J=15.1 Hz, 1H), 1.30 (dd, J=6.0, 1.7 Hz, 6H), 1.20 (s, 3H), 0.61 (s, 12H), 0.56-0.42 (m, 3H). ESI-MS m/z calc. 714.3563, found 715.26 (M+1)⁺; Retention time: 2.21 minutes. LC method A.

### C. Characterization of Compounds

The compounds in Table 22 were prepared by procedures analogous to those disclosed in the specification, and the analytical data were consistent with the reported structure.

TABLE 22
LCMS data

| Compound | | LCMS | Calc. | | LCMS |
|---|---|---|---|---|---|
| No. | Structure | Rt (min) | mass | M + 1 | Method |
| 331 | 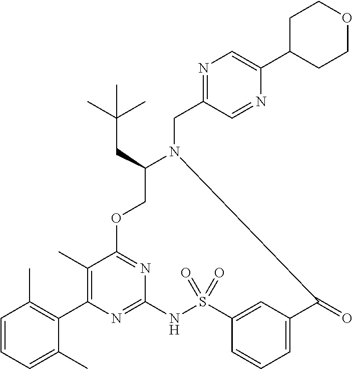 [SMILES: Cc1cccc(C)c1-c1nc2nc(c1C)OC[C@@H](CC(C)(C)C)N(Cc1cnc(C3CCOCC3)cn1)C(=O)c1cccc(c1)S(=O)(=O)N2] | 1.8 | 684.309 | 685.6 | A |
| 332 | 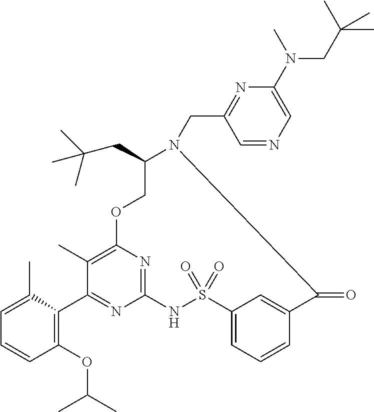 | 2.06 | 743.383 | 744.69 | A |
| 333 | 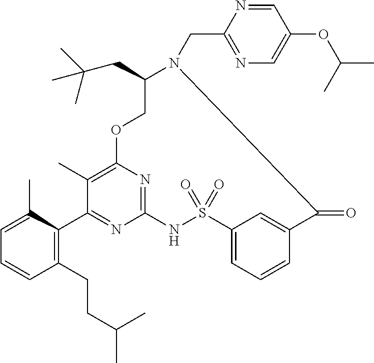 | 2.21 | 714.356 | 715.26 | A |
| 334 | 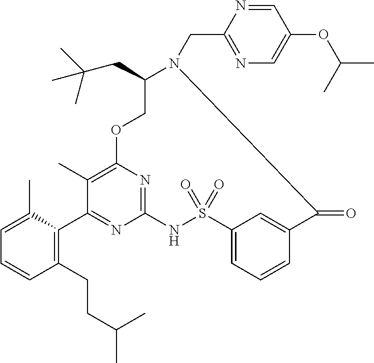 | 2.26 | 714.356 | 715.7 | A |
| 335 | 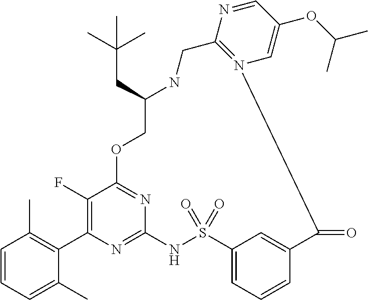 | 2.085 | 662.269 | 663.4 | A |
| 336 | 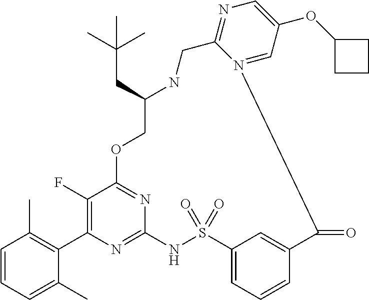 | 2.125 | 674.269 | 675.45 | A |
| 337 | 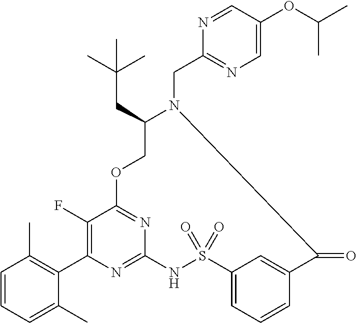 [SMILES: Cc1cccc(C)c1-c1nc2nc(c1F)OC[C@@H](CC(C)(C)C)N(Cc1ncc(OC(C)C)cn1)C(=O)c1cccc(c1)S(=O)(=O)N2] | 2.04 | 648.253 | 649.4 | A |
| 338 | 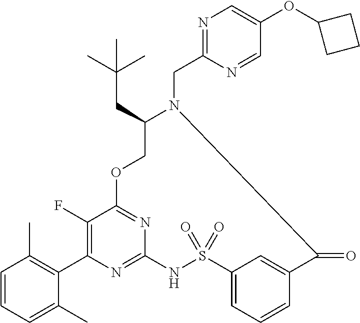 [SMILES: Cc1cccc(C)c1-c1nc2nc(c1F)OC[C@@H](CC(C)(C)C)N(Cc1ncc(OC3CCC3)cn1)C(=O)c1cccc(c1)S(=O)(=O)N2] | 2.075 | 660.253 | 661.4 | A |
| 339 | 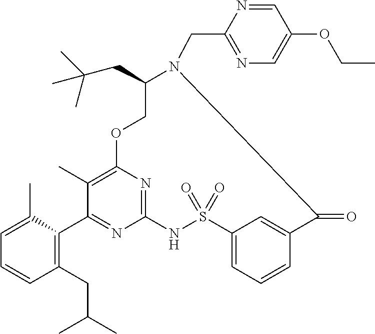 | 2.09 | 686.325 | 687.59 | A |
| 340 | 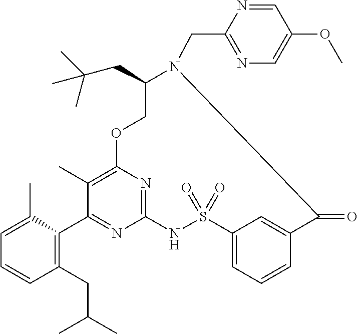 | 2.01 | 672.309 | 673.56 | A |
| 341 | 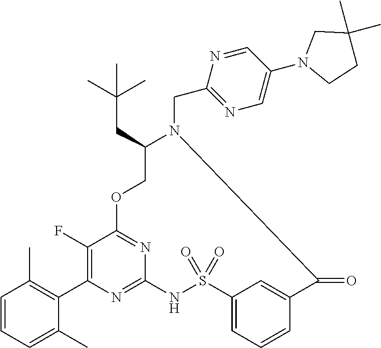 [SMILES: Cc1cccc(C)c1-c1nc2nc(c1F)OC[C@@H](CC(C)(C)C)N(Cc1ncc(N3CCC(C)(C)C3)cn1)C(=O)c1cccc(c1)S(=O)(=O)N2] | 2.14 | 701.316 | 702.5 | A |
| 342 | 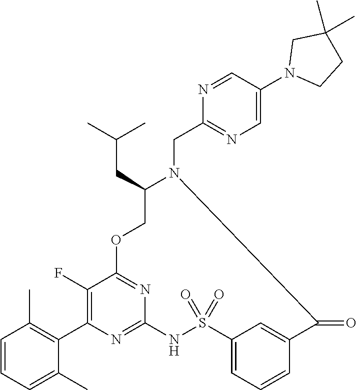 [SMILES: Cc1cccc(C)c1-c1nc2nc(c1F)OC[C@@H](CC(C)C)N(Cc1ncc(N3CCC(C)(C)C3)cn1)C(=O)c1cccc(c1)S(=O)(=O)N2] | 2.09 | 687.3 | 688.5 | A |
| 343 | 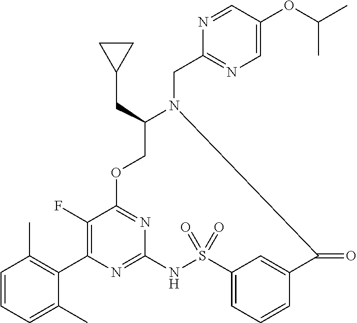 [SMILES: Cc1cccc(C)c1-c1nc2nc(c1F)OC[C@@H](CC1CC1)N(Cc1ncc(OC(C)C)cn1)C(=O)c1cccc(c1)S(=O)(=O)N2] | 1.35 | 646.237 | 647.4 | A |
| 344 | 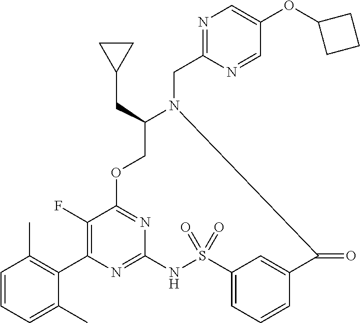 [SMILES: Cc1cccc(C)c1-c1nc2nc(c1F)OC[C@@H](CC1CC1)N(Cc1ncc(OC3CCC3)cn1)C(=O)c1cccc(c1)S(=O)(=O)N2] | 2 | 658.237 | 659.4 | A |
| 345 | 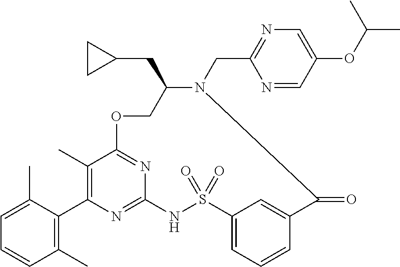 [SMILES: Cc1cccc(C)c1-c1nc2nc(c1C)OC[C@@H](CC1CC1)N(Cc1ncc(OC(C)C)cn1)C(=O)c1cccc(c1)S(=O)(=O)N2] | 1.79 | 642.262 | 643.6 | A |
| 346 | 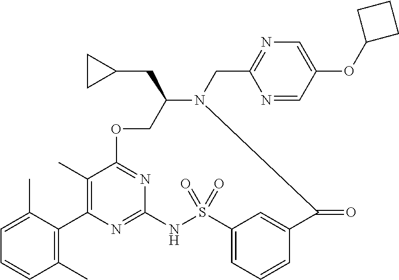 [SMILES: Cc1cccc(C)c1-c1nc2nc(c1C)OC[C@@H](CC1CC1)N(Cc1ncc(OC3CCC3)cn1)C(=O)c1cccc(c1)S(=O)(=O)N2] | 1.85 | 654.262 | 655.6 | A |
| 347 | 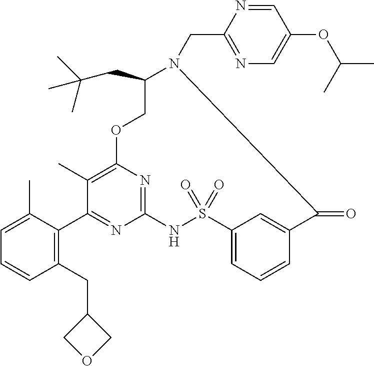 [SMILES: Cc1cccc(CC2COC2)c1-c1nc2nc(c1C)OC[C@@H](CC(C)(C)C)N(Cc1ncc(OC(C)C)cn1)C(=O)c1cccc(c1)S(=O)(=O)N2] | 1.8 | 714.32 | 715.64 | A |
| 348 | 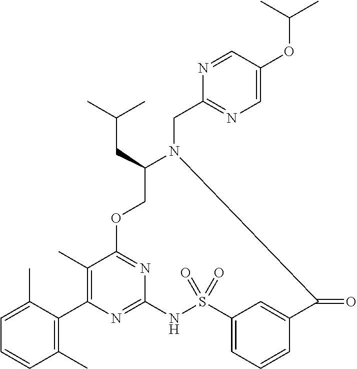 [SMILES: Cc1cccc(C)c1-c1nc2nc(c1C)OC[C@@H](CC(C)C)N(Cc1ncc(OC(C)C)cn1)C(=O)c1cccc(c1)S(=O)(=O)N2] | 1.91 | 660.273 | 661.4 | A |
| 349 | 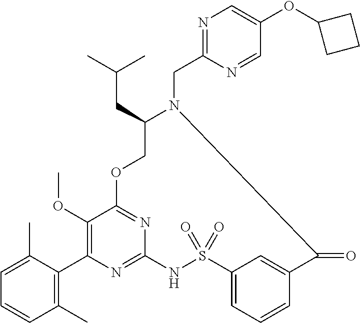 [SMILES: COc1c2nc(nc1-c1c(C)cccc1C)NS(=O)(=O)c1cccc(c1)C(=O)N(Cc1ncc(OC3CCC3)cn1)[C@H](CC(C)C)CO2] | 1.95 | 672.273 | 673.5 | A |
| 350 | 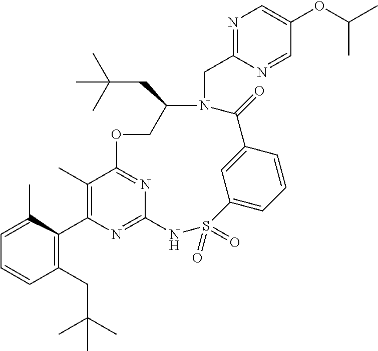 | 2.18 | 714.356 | 715.7 | A |
| 351 | 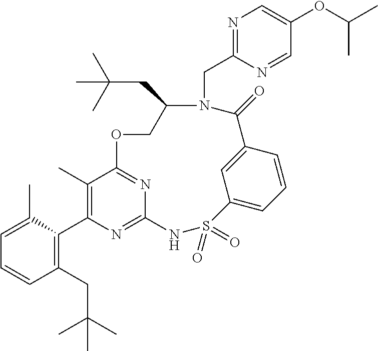 | 2.22 | 714.356 | 715.7 | A |
| 352 | 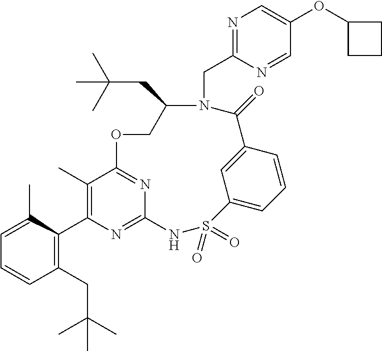 | 2.22 | 726.356 | 727.9 | A |
| 353 | 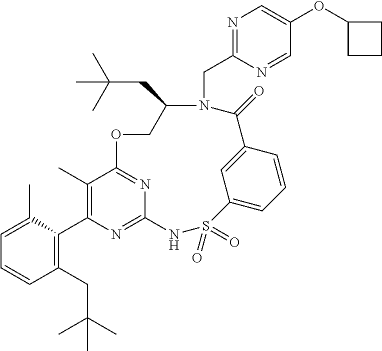 | 2.26 | 726.356 | 728 | A |
| 354 | 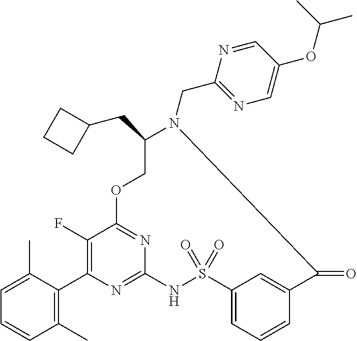 [SMILES: Cc1cccc(C)c1-c1nc2nc(c1F)OC[C@@H](CC1CCC1)N(Cc1ncc(OC(C)C)cn1)C(=O)c1cccc(c1)S(=O)(=O)N2] | 2.06 | 660.253 | 661.4 | A |
| 355 | 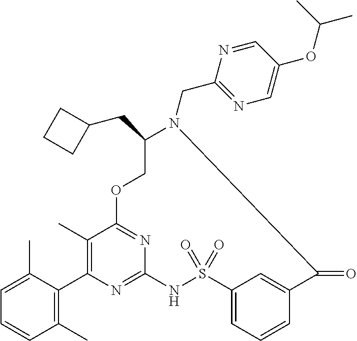 [SMILES: Cc1cccc(C)c1-c1nc2nc(c1C)OC[C@@H](CC1CCC1)N(Cc1ncc(OC(C)C)cn1)C(=O)c1cccc(c1)S(=O)(=O)N2] | 1.95 | 656.278 | 657.1 | A |
| 356 | 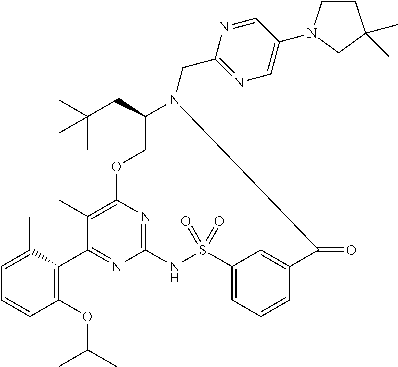 | 2.08 | 741.367 | 742.6 | A |
| 357 | 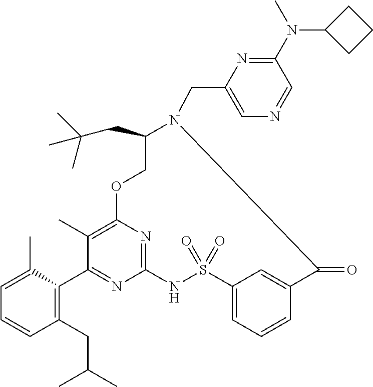 | 2.17 | 725.372 | 726.93 | A |
| 358 | 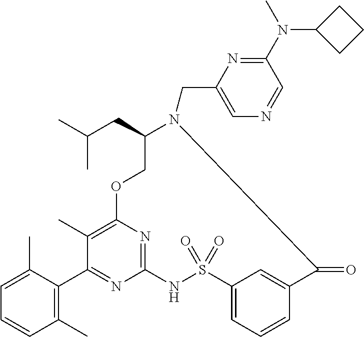 [SMILES: Cc1cccc(C)c1-c1nc2nc(c1C)OC[C@@H](CC(C)C)N(Cc1cncc(N(C)C3CCC3)n1)C(=O)c1cccc(c1)S(=O)(=O)N2] | 1.81 | 669.31 | 670.2 | A |
| 359 | 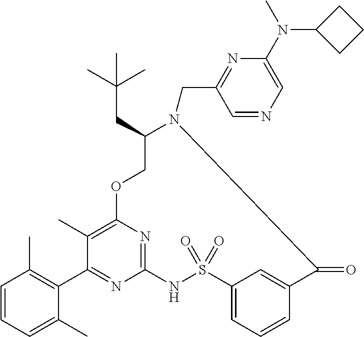 [SMILES: Cc1cccc(C)c1-c1nc2nc(c1C)OC[C@@H](CC(C)(C)C)N(Cc1cncc(N(C)C3CCC3)n1)C(=O)c1cccc(c1)S(=O)(=O)N2] | 1.92 | 683.325 | 684.9 | A |
| 360 | 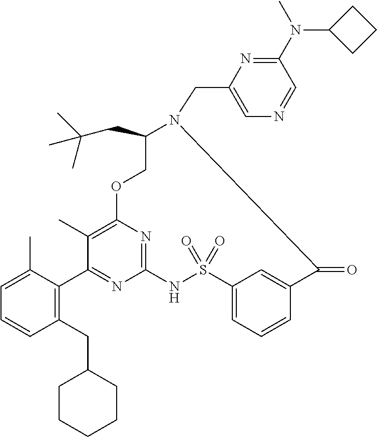 [SMILES: Cc1cccc(CC2CCCCC2)c1-c1nc2nc(c1C)OC[C@@H](CC(C)(C)C)N(Cc1cncc(N(C)C3CCC3)n1)C(=O)c1cccc(c1)S(=O)(=O)N2] | 1.92 | 765.404 | 766.99 | A (30-99% gradient) |
| 361 | 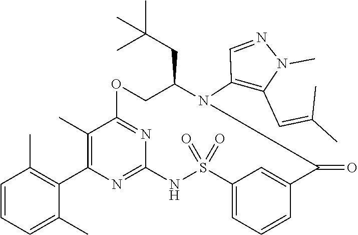 [SMILES: CC(C)=Cc1c(N2C(=O)c3cccc(c3)S(=O)(=O)Nc3nc(c(C)c(-c4c(C)cccc4C)n3)OC[C@H]2CC(C)(C)C)cnn1C] | 4.42 | 642.299 | 643.3 | Y |
| 362 | 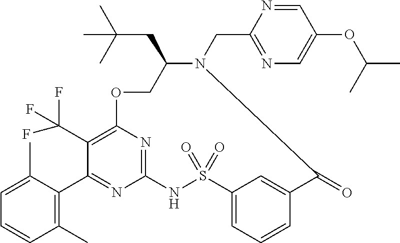 [SMILES: Cc1cccc(C)c1-c1nc2nc(c1C(F)(F)F)OC[C@@H](CC(C)(C)C)N(Cc1ncc(OC(C)C)cn1)C(=O)c1cccc(c1)S(=O)(=O)N2] | 3.24 | 711.27 | 712.1 | 1D |
| 363 | 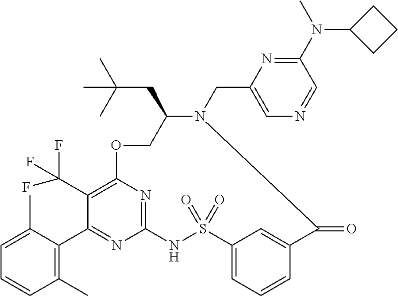 [SMILES: Cc1cccc(C)c1-c1nc2nc(c1C(F)(F)F)OC[C@@H](CC(C)(C)C)N(Cc1cncc(N(C)C3CCC3)n1)C(=O)c1cccc(c1)S(=O)(=O)N2] | 3.31 | 736.302 | 737.3 | 1D |
| 364 | 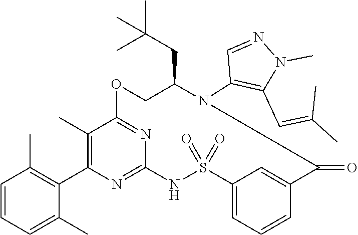 [SMILES: CC(C)=Cc1c(N2C(=O)c3cccc(c3)S(=O)(=O)Nc3nc(c(C)c(-c4c(C)cccc4C)n3)OC[C@H]2CC(C)(C)C)cnn1C] | 4.39 | 701.316 | 702.8 | A |
| 365 | 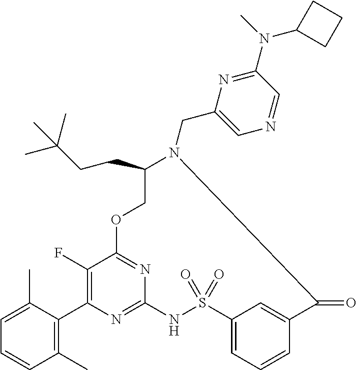 [SMILES: Cc1cccc(C)c1-c1nc2nc(c1F)OC[C@@H](CCC(C)(C)C)N(Cc1cncc(N(C)C3CCC3)n1)C(=O)c1cccc(c1)S(=O)(=O)N2] | 2.11 | 701.316 | 702.8 | A |
| 366 | 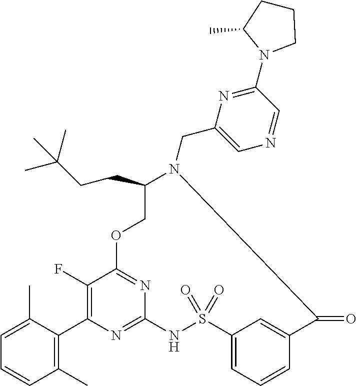 [SMILES: Cc1cccc(C)c1-c1nc2nc(c1F)OC[C@@H](CCC(C)(C)C)N(Cc1cncc(N3CCC[C@H]3C)n1)C(=O)c1cccc(c1)S(=O)(=O)N2] | 2.05 | 701.316 | 702.8 | A |
| 367 | 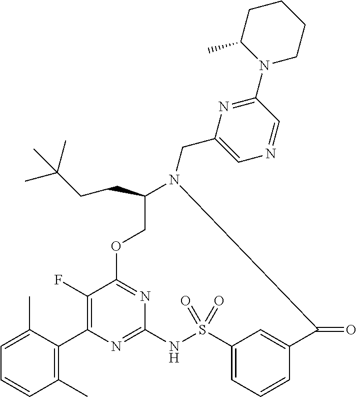 [SMILES: Cc1cccc(C)c1-c1nc2nc(c1F)OC[C@@H](CCC(C)(C)C)N(Cc1cncc(N3CCCC[C@H]3C)n1)C(=O)c1cccc(c1)S(=O)(=O)N2] | 2.12 | 715.332 | 716.80 | A |
| 368 | 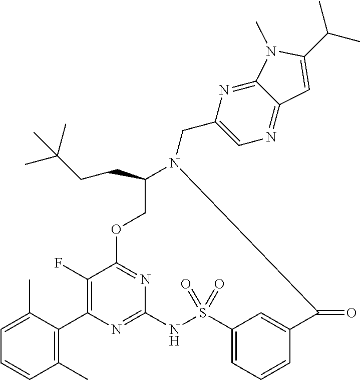 [SMILES: Cc1cccc(C)c1-c1nc2nc(c1F)OC[C@@H](CCC(C)(C)C)N(Cc1cnc3cc(C(C)C)n(C)c3n1)C(=O)c1cccc(c1)S(=O)(=O)N2] | 2.04 | 713.316 | 714.9 | A |
| 369 | 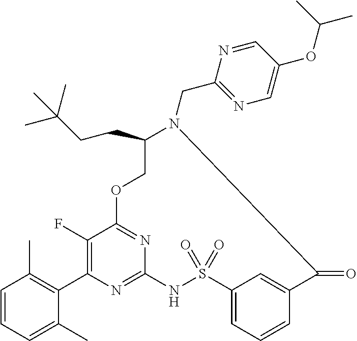 [SMILES: Cc1cccc(C)c1-c1nc2nc(c1F)OC[C@@H](CCC(C)(C)C)N(Cc1ncc(OC(C)C)cn1)C(=O)c1cccc(c1)S(=O)(=O)N2] | 2.15 | 676.284 | 677.8 | A |

TABLE 23
NMR data

| Compound | |
|---|---|
| number | NMR |
| 331 | ¹H NMR (400 MHz, DMSO) δ 8.69 (d, J = 1.5 Hz, 1H), 8.59 (t, |
| | J = 2.3 Hz, 2H), 8.00-7.87 (m, 1H), 7.75-7.58 (m, 2H), 7.28 (t, |
| | J = 7.6 Hz, 1H), 7.18 (d, J = 7.6 Hz, 1H), 7.13 (d, J = 7.6 Hz, |
| | 1H), 5.36 (dd, J = 11.3, 4.4 Hz, 1H), 4.86 (d, J = 15.7 Hz, 1H), |
| | 4.63 (d, J = 15.7 Hz, 1H), 4.40 (t, J = 11.2 Hz, 1H), 4.10-4.00 |
| | (m, 1H), 3.96 (dt, J = 11.0, 3.2 Hz, 2H), 3.53-3.43 (m, 2H), |
| | 3.04 (ddd, J = 15.5, 9.2, 7.1 Hz, 1H), 2.06 (s, 3H), 1.86-1.74 |
| | (m, 8H), 1.62 (s, 3H), 1.41 (d, J = 15.0 Hz, 1H), 0.54 (s, 9H). |
| 332 | ¹H NMR (400 MHz, DMSO-d₆) δ 13.22-11.45 (broad m, 1H), |
| | 8.66 (br s, 1H), 8.07 (s, 1H), 7.91 (br s, 1H), 7.84 (s, 1H), 7.65 |
| | (br s, 2H), 7.32 (s, 1H), 6.99 (d, J = 8.3 Hz, 1H), 6.84 (d, J = 7.5 |
| | Hz, 1H), 5.40 (br s, 1H), 4.74 (d, J = 15.7 Hz, 1H), 4.60 (s, 1H), |
| | 4.43 (d, J = 15.7 Hz, 1H), 4.39-4.26 (m, 1H), 4.11-3.96 (m, |
| | 1H), 3.55-3.39 (m, 2H), 3.16 (s, 3H), 1.87-1.68 (m, 4H), 1.62 |
| | (s, 3H), 1.38 (d, J = 14.8 Hz, 1H), 1.19 (d, J = 5.8 Hz, 3H), 1.15 |
| | (d, J = 6.0 Hz, 3H), 0.92 (s, 9H), 0.52 (s, 9H). |
| 333 | test, ¹H NMR (400 MHz, DMSO-d₆) δ 13.34-11.61 (broad m, |
| | 1H), 8.68 (br s, 1H), 8.52 (s, 2H), 7.86 (br s, 1H), 7.62 (br s, 2H), |
| | 7.30 (br s, 1H), 7.22-7.01 (m, 2H), 5.45 (br s, 1H), 4.94-4.75 |
| | (m, 2H), 4.66 (d, J = 16.5 Hz, 1H), 4.19 (t, J = 11.3 Hz, 1H), |
| | 3.92 (td, J = 10.5, 9.8, 4.2 Hz, 1H), 2.16 (s, 1H), 2.04 (s, 3H), |
| | 1.86 (dd, J = 15.2, 9.6 Hz, 2H), 1.58 (s, 3H), 1.37 (d, J = 15.1 |
| | Hz, 1H), 1.30 (dd, J = 6.0, 1.7 Hz, 6H), 1.20 (s, 3H), 0.61 (s, |
| | 12H), 0.56-0.42 (m, 3H). |
| 334 | ¹H NMR (400 MHz, DMSO-d₆) δ 13.35-11.54 (broad m, 1H), |
| | 8.70 (br s, 1H), 8.52 (s, 2H), 7.94 (br s, 1H), 7.66 (br s, 2H), 7.30 |
| | (s, 1H), 7.15 (d, J = 25.5 Hz, 2H), 5.42 (s, 1H), 4.87 (d, J = 16.5 |
| | Hz, 1H), 4.81 (p, J = 6.0 Hz, 1H), 4.65 (d, J = 16.5 Hz, 1H), |
| | 4.37-4.13 (m, 1H), 4.13-3.96 (m, 1H), 2.39-2.15 (m, 2H), |
| | 1.95-1.69 (m, 4H), 1.61 (s, 3H), 1.49-1.19 (m, 10H), 0.79 (s, |
| | 6H), 0.54 (s, 9H). |
| 335 | ¹H NMR (400 MHz, Methanol-d₄) δ 8.91 (d, J = 1.9 Hz, 1H), |
| | 8.48 (s, 2H), 8.13 (dt, J = 7.7, 1.6 Hz, 1H), 7.87-7.67 (m, 2H), |
| | 7.23 (t, J = 7.6 Hz, 1H), 7.10 (dd, J = 18.7, 7.5 Hz, 2H), 5.61 |
| | (dd, J = 10.6, 3.9 Hz, 1H), 5.05 (d, J = 16.5 Hz, 1H), 4.66 (d, |
| | J = 16.6 Hz, 1H), 4.30-4.10 (m, 2H), 2.14 (s, 3H), 1.95-1.82 (m, |
| | 4H), 1.60 (dd, J = 15.4, 1.5 Hz, 1H), 1.36 (dd, J = 6.0, 3.5 Hz, |
| | 6H), 1.33-1.22 (m, 2H), 0.65 (s, 9H). |
| 336 | ¹H NMR (400 MHz, Methanol-d₄) δ 8.96-8.86 (m, 1H), 8.41 (s, |
| | 2H), 8.13 (dt, J = 7.6, 1.6 Hz, 1H), 7.86-7.69 (m, 2H), 7.23 (t, |
| | J = 7.6 Hz, 1H), 7.10 (dd, J = 18.9, 7.6 Hz, 2H), 5.61 (dd, J = |
| | 10.6, 3.8 Hz, 1H), 5.05 (d, J = 16.5 Hz, 1H), 4.65 (d, J = 16.6 |
| | Hz, 1H), 4.30-4.08 (m, 2H), 2.51 (dddd, J = 9.3, 8.1, 4.0, 2.5 |
| | Hz, 2H), 2.21-2.08 (m, 5H), 1.92-1.82 (m, 5H), 1.80-1.68 (m, |
| | 1H), 1.60 (dd, J = 15.3, 1.5 Hz, 1H), 1.34-1.24 (m, 1H), 0.65 (s, 9H). |
| 337 | ¹H NMR (400 MHz, Methanol-d₄) δ 8.85 (t, J = 1.8 Hz, 1H), |
| | 8.48 (s, 2H), 8.10 (dt, J = 7.7, 1.5 Hz, 1H), 7.85-7.68 (m, 2H), |
| | 7.23 (t, J = 7.6 Hz, 1H), 7.10 (dd, J = 15.6, 7.6 Hz, 2H), 5.58 |
| | (dd, J = 10.9, 4.1 Hz, 1H), 5.07 (d, J = 16.6 Hz, 1H), 4.78-4.72 |
| | (m, 1H), 4.63 (d, J = 16.6 Hz, 1H), 4.26 (t, J = 11.3 Hz, 1H), |
| | 4.16-4.05 (m, 1H), 2.13 (s, 3H), 1.94 (s, 3H), 1.84 (ddd, J = |
| | 13.9, 10.3, 3.3 Hz, 1H), 1.55-1.46 (m, 1H), 1.36 (dd, J = 6.0, |
| | 3.2 Hz, 7H), 0.83 (d, J = 6.6 Hz, 3H), 0.38 (d, J = 6.4 Hz, 3H). |
| 338 | ¹H NMR (400 MHz, Methanol-d₄) δ 8.84 (t, J = 1.8 Hz, 1H), |
| | 8.41 (s, 2H), 8.10 (dt, J = 7.7, 1.6 Hz, 1H), 7.85-7.66 (m, 2H), |
| | 7.23 (t, J = 7.6 Hz, 1H), 7.10 (dd, J = 15.7, 7.6 Hz, 2H), 5.57 |
| | (dd, J = 10.9, 4.1 Hz, 1H), 5.07 (d, J = 16.6 Hz, 1H), 4.63 (d, |
| | J = 16.6 Hz, 1H), 4.25 (t, J = 11.3 Hz, 1H), 4.18-4.03 (m, 1H), |
| | 2.61-2.43 (m, 2H), 2.26-2.09 (m, 5H), 1.94 (s, 3H), 1.92-1.70 |
| | (m, 3H), 1.56-1.43 (m, 1H), 1.43-1.33 (m, 1H), 1.24 (t, J = 7.1 |
| | Hz, 1H), 0.83 (d, J = 6.5 Hz, 3H), 0.38 (d, J = 6.4 Hz, 3H). |
| 339 | ¹H NMR (400 MHz, DMSO-d₆) δ 13.33-11.66 (broad m, 1H), |
| | 8.70 (s, 1H), 8.52 (s, br 2H), 7.93 (s, 1H), 7.65 (s, 2H), 7.31 (s, |
| | 1H), 7.15 (s, 2H), 5.43 (s, 1H), 4.89 (d, J = 16.5 Hz, 1H), 4.66 |
| | (d, J = 16.6 Hz, 1H), 4.30-4.15 (m, 3H), 4.10-3.95 (m, 1H), |
| | 2.30-2.11 (m, 2H), 1.94-1.67 (m, 5H), 1.58 (s, 3H), 1.43-1.29 |
| | (m, 4H), 0.89-0.68 (m, 6H), 0.54 (s, 9H). |
| 340 | ¹H NMR (400 MHz, DMSO-d₆) δ 13.18-11.61 (broad m, 1H), |
| | 8.71 (s, 1H), 8.55 (s, 2H), 7.93 (s, 1H), 7.64 (br s, 2H), 7.31 (s, |
| | 1H), 7.15 (s, 2H), 5.44 (s, 1H), 4.90 (d, J = 16.5 Hz, 1H), 4.67 |
| | (d, J = 16.6 Hz, 1H), 4.30-4.13 (m, 1H), 4.10-3.97 (m, 1H), |
| | 3.92 (s, 3H), 2.28-2.10 (m, 2H), 1.88-1.66 (m, 5H), 1.58 (s, |
| | 3H), 1.38 (d, J = 15.1 Hz, 1H), 0.86-0.68 (m, 6H), 0.55 (s, 9H). |
| 341 | ¹H NMR (400 MHz, DMSO-d₆) δ 12.17 (s, 1H), 8.72 (s, 1H), |
| | 8.07 (s, 2H), 8.05-8.00 (m, 1H), 7.79-7.75 (m, 2H), 7.27 (t, |
| | J = 7.6 Hz, 1H), 7.14 (dd, J = 21.6, 7.6 Hz, 2H), 5.38 (dd, J = |
| | 11.0, 4.3 Hz, 1H), 4.85 (d, J = 16.3 Hz, 1H), 4.57 (d, J = 16.3 |
| | Hz, 1H), 4.29 (t, J = 11.3 Hz, 1H), 4.00-3.95 (m, 1H), 3.08 (s, |
| | 2H), 2.10 (s, 3H), 1.84 (s, 4H), 1.77 (t, J = 6.9 Hz, 2H), 1.40 (d, |
| | J = 14.9 Hz, 1H), 1.28-1.13 (m, 2H), 1.10 (s, 6H), 0.56 (s, 9H). |
| 342 | ¹H NMR (400 MHz, DMSO-d₆) δ 12.14 (s, 1H), 8.65 (s, 1H), |
| | 8.08 (s, 2H), 8.03-7.98 (m, 1H), 7.80-7.74 (m, 2H), 7.27 (t, |
| | J = 7.6 Hz, 1H), 7.14 (dd, J = 17.7, 7.6 Hz, 2H), 5.34 (dd, J = |
| | 10.9, 4.3 Hz, 1H), 4.89 (d, J = 16.2 Hz, 1H), 4.53 (d, J = 16.3 |
| | Hz, 1H), 4.28 (t, J = 11.2 Hz, 1H), 4.01-3.91 (m, 1H), 3.39 (t, |
| | J = 7.0 Hz, 2H), 3.08 (s, 2H), 2.09 (s, 3H), 1.89 (s, 3H), 1.77 (t, |
| | J = 7.0 Hz, 2H), 1.47-1.33 (m, 1H), 1.28-1.16 (m, 2H), 1.10 (s, |
| | 6H), 0.76 (d, J = 6.6 Hz, 3H), 0.28 (d, J = 6.4 Hz, 3H). |
| 343 | ¹H NMR (400 MHz, DMSO-d₆) δ 12.10 (s, 1H), 8.63 (s, 1H), |
| | 8.53 (s, 2H), 8.00 (dt, J = 7.1, 1.8 Hz, 1H), 7.84-7.71 (m, 2H), |
| | 7.31-7.22 (m, 1H), 7.13 (dd, J = 14.8, 7.6 Hz, 2H), 5.32 (dd, |
| | J = 11.0, 4.2 Hz, 1H), 4.98 (d, J = 16.6 Hz, 1H), 4.82 (hept, J = |
| | 5.9 Hz, 1H), 4.62 (d, J = 16.6 Hz, 1H), 4.34 (t, J = 11.4 Hz, 1H), |
| | 4.12-3.97 (m, 1H), 2.08 (s, 3H), 1.93 (s, 3H), 1.30 (dd, J = 6.0, |
| | 1.6 Hz, 6H), 1.18-1.11 (m, 1H), 0.52-0.41 (m, 1H), 0.42-0.32 |
| | (m, 1H), 0.24-0.11 (m, 1H), −0.00 (s, 2H), −0.48-−0.60 (m, 1H). |
| 344 | ¹H NMR (400 MHz, DMSO-d₆) δ 12.10 (s, 1H), 8.63 (d, J = 1.9 |
| | Hz, 1H), 8.46 (s, 2H), 8.00 (dt, J = 7.0, 1.9 Hz, 1H), 7.83-7.73 |
| | (m, 2H), 7.27 (t, J = 7.6 Hz, 1H), 7.13 (dd, J = 15.0, 7.6 Hz, |
| | 2H), 5.32 (dd, J = 11.0, 4.2 Hz, 1H), 4.97 (d, J = 16.6 Hz, 1H), |
| | 4.89 (p, J = 7.1 Hz, 1H), 4.62 (d, J = 16.6 Hz, 1H), 4.35 (t, J = |
| | 11.3 Hz, 1H), 4.11-4.01 (m, 1H), 2.10-2.05 (m, 4H), 1.93 (s, |
| | 3H), 1.86-1.75 (m, 2H), 1.76-1.57 (m, 2H), 1.29-1.11 (m, |
| | 3H), 0.90-0.75 (m, 1H), 0.55-0.42 (m, 1H), 0.41-0.31 (m, |
| | 1H), 0.23-0.11 (m, 1H), −0.47-−0.61 (m, 1H). |
| 345 | ¹H NMR (400 MHz, DMSO-d₆) δ 13.26-12.47 (bs, 1H), 8.62 (s, |
| | 1H), 8.53 (s, 2H), 7.91 (s, 1H), 7.66 (s, 2H), 7.27 (t, J = 7.6 Hz, |
| | 1H), 7.16 (d, J = 7.7 Hz, 1H), 7.13 (d, J = 7.6 Hz, 1H), 5.45- |
| | 5.30 (m, 1H), 4.98 (d, J = 16.5 Hz, 1H), 4.81 (hept, J = 6.0 Hz, |
| | 1H), 4.59 (d, J = 16.5 Hz, 1H), 4.22 (t, J = 11.2 Hz, 1H), 4.09- |
| | 3.98 (m, 1H), 2.02 (s, 3H), 1.96-1.77 (m, 4H), 1.55 (s, 3H), 1.31 |
| | (dd, J = 6.0, 1.6 Hz, 6H), 1.20-1.08 (m, 1H), 0.51-0.41 (m, |
| | 1H), 0.41-0.32 (m, 1H), 0.22-0.11 (m, 1H), 0.06-0.00 (m, |
| | 1H), −0.48-−0.68 (m, 1H) |
| 346 | ¹H NMR (400 MHz, DMSO-d₆) δ 13.31-12.61 (bs, 1H), 8.62 (s, |
| | 1H), 8.45 (s, 2H), 7.91 (s, 1H), 7.65 (s, 2H), 7.27 (t, J = 7.6 Hz, |
| | 1H), 7.16 (d, J = 7.7 Hz, 1H), 7.13 (d, J = 7.7 Hz, 1H), 5.45- |
| | 5.29 (m, 1H), 4.97 (d, J = 16.5 Hz, 1H), 4.89 (p, J = 7.1 Hz, |
| | 1H), 4.60 (d, J = 16.5 Hz, 1H), 4.23 (t, J = 11.2 Hz, 1H), 4.05 |
| | (tt, J = 11.2, 4.0 Hz, 1H), 2.48-2.41 (m, 2H), 2.17-2.03 (m, |
| | 2H), 2.03 (s, 3H), 1.88 (s, 3H), 1.88-1.74 (m, 2H), 1.71-1.54 |
| | (m, 1H), 1.54 (s, 3H), 1.20-1.03 (m, 1H), 0.51-0.40 (m, 1H), |
| | 0.40-0.31 (m, 1H), 0.24-0.10 (m, 1H), 0.07-−0.02 (m, |
| | 1H), −0.44-−0.70 (m, 1H) |
| 348 | ¹H NMR (400 MHz, Methanol-d₄) δ 8.89-8.78 (m, 1H), 8.48 (t, |
| | 2H), 8.05 (dt, J = 7.6, 1.6 Hz, 1H), 7.77-7.65 (m, 2H), 7.24 (t, |
| | J = 7.6 Hz, 1H), 7.11 (dd, J = 17.3, 7.6 Hz, 2H), 5.61 (dd, J = |
| | 10.5, 3.8 Hz, 1H), 5.08 (d, J = 16.6 Hz, 1H), 4.80-4.71 (m, 2H), |
| | 4.62 (d, J = 16.6 Hz, 1H), 4.29-4.03 (m, 2H), 3.44 (s, 3H), 2.16 |
| | (s, 3H), 1.97 (s, 3H), 1.37 (dd, J = 6.0, 3.2 Hz, 7H), 1.24 (t, J = |
| | 7.1 Hz, 1H), 0.84 (d, J = 6.6 Hz, 3H), 0.40 (d, J = 6.4 Hz, 3H). |
| 349 | ¹H NMR (400 MHz, Methanol-d₄) δ 8.83 (t, J = 1.8 Hz, 1H), |
| | 8.41 (s, 2H), 8.05 (dt, J = 7.5, 1.7 Hz, 1H), 7.80-7.62 (m, 2H), |
| | 7.25 (t, J = 7.6 Hz, 1H), 7.12 (dd, J = 17.5, 7.5 Hz, 2H), 5.61 |
| | (dd, J = 10.5, 3.8 Hz, 1H), 5.07 (d, J = 16.5 Hz, 1H), 4.62 (d, |
| | J = 16.6 Hz, 1H), 4.31-4.06 (m, 2H), 3.44 (s, 3H), 2.52 (dddd, |
| | J = 12.6, 7.2, 6.2, 2.4 Hz, 2H), 2.20-2.09 (m, 5H), 1.97 (s, 3H), |
| | 1.93-1.67 (m, 3H), 1.51 (ddt, J = 13.0, 6.5, 3.4 Hz, 1H), 1.44- |
| | 1.33 (m, 2H), 1.33-1.19 (m, 1H), 0.84 (d, J = 6.5 Hz, 3H), 0.40 |
| | (d, J = 6.4 Hz, 3H). |
| 350 | ¹H NMR (500 MHz, DMSO-d₆) δ 12.98 (s, 1H), 8.70 (s, 1H), |
| | 8.52 (s, 2H), 7.89 (s, 1H), 7.64 (s, 2H), 7.33 (s, 1H), 7.22 (s, 1H), |
| | 7.15 (d, J = 7.7 Hz, 1H), 5.44 (s, 1H), 4.89 (d, J = 16.5 Hz, 1H), |
| | 4.81 (h, J = 6.0 Hz, 1H), 4.66 (d, J = 16.5 Hz, 1H), 4.27-4.14 |
| | (m, 1H), 4.09-3.96 (m, 1H), 2.27 (d, J = 13.3 Hz, 1H), 2.08 (s, |
| | 3H), 1.97 (d, J = 13.3 Hz, 1H), 1.86 (dd, J = 15.3, 9.4 Hz, 1H), |
| | 1.58 (s, 3H), 1.36 (d, J = 15.2 Hz, 1H), 1.32 (d, J = 6.7 Hz, 3H), |
| | 1.30 (d, J = 6.7 Hz, 3H), 0.64 (s, 9H), 0.61 (s, 9H). |
| 351 | ¹H NMR (500 MHz, DMSO-d₆) δ 12.97 (s, 1H), 8.72 (s, 1H), |
| | 8.52 (s, 2H), 7.94 (d, J = 7.6 Hz, 1H), 7.69 (t, J = 7.6 Hz, 1H), |
| | 7.65 (d, J = 7.6 Hz, 1H), 7.32 (t, J = 7.7 Hz, 1H), 7.18 (d, J = |
| | 7.8 Hz, 1H), 7.16 (d, J = 7.5 Hz, 1H), 5.45 (dd, J = 10.9, 4.4 Hz, |
| | 1H), 4.90 (d, J = 16.5 Hz, 1H), 4.81 (hept, J = 6.1 Hz, 1H), 4.65 |
| | (d, J = 16.6 Hz, 1H), 4.23 (t, J = 11.2 Hz, 1H), 4.07-4.00 (m, |
| | 1H), 2.42 (d, J = 13.4 Hz, 1H), 2.25 (d, J = 13.4 Hz, 1H), 1.86- |
| | 1.81 (m, 1H), 1.80 (s, 3H), 1.59 (s, 3H), 1.38 (d, J = 15.2 Hz, |
| | 1H), 1.32 (d, J = 6.6 Hz, 3H), 1.30 (d, J = 6.6 Hz, 3H), 0.82 (s, |
| | 9H), 0.55 (s, 9H). |
| 352 | ¹H NMR (400 MHz, DMSO-d₆) δ 12.99 (s, 1H), 8.68 (s, 1H), |
| | 8.44 (s, 2H), 7.88 (s, 1H), 7.63 (s, 2H), 7.32 (s, 1H), 7.22 (s, 1H), |
| | 7.14 (d, J = 7.7 Hz, 1H), 5.42 (s, 1H), 4.94-4.80 (m, 2H), 4.66 |
| | (d, J = 16.5 Hz, 1H), 4.22 (t, J = 10.9 Hz, 1H), 4.11-3.94 (m, |
| | 1H), 2.47-2.43 (m, 1H), 2.26 (d, J = 13.3 Hz, 1H), 2.19-2.01 |
| | (m, 5H), 1.96 (d, J = 13.3 Hz, 1H), 1.88-1.74 (m, 2H), 1.71- |
| | 1.58 (m, 2H), 1.56 (s, 3H), 1.34 (d, J = 15.1 Hz, 1H), 0.63 (s, |
| | 9H), 0.60 (s, 9H). |
| 353 | ¹H NMR (400 MHz, DMSO-d₆) δ 13.01 (s, 1H), 8.70 (s, 1H), |
| | 8.44 (s, 2H), 7.92 (s, 1H), 7.65 (s, 2H), 7.31 (s, 1H), 7.18 (d, J = |
| | 7.9 Hz, 2H), 5.44 (s, 1H), 4.98-4.80 (m, 2H), 4.65 (d, J = 16.6 |
| | Hz, 1H), 4.23 (s, 1H), 4.03 (s, 1H), 2.47-2.36 (m, 2H), 2.24 (d, |
| | J = 13.4 Hz, 1H), 2.15-1.98 (m, 2H), 1.92-1.72 (m, 5H), 1.65 |
| | (tt, J = 10.5, 8.2 Hz, 2H), 1.57 (s, 3H), 1.36 (d, J = 15.1 Hz, 1H), |
| | 0.81 (s, 9H), 0.54 (s, 9H). |
| 354 | ¹H NMR (400 MHz, DMSO-d₆) δ 12.13 (s, 1H), 8.61 (d, J = 1.9 |
| | Hz, 1H), 8.52 (s, 2H), 7.99 (dt, J = 7.3, 1.8 Hz, 1H), 7.86-7.73 |
| | (m, 2H), 7.28 (t, J = 7.6 Hz, 1H), 7.15 (dd, J = 15.3, 7.6 Hz, |
| | 2H), 5.32 (dd, J = 10.9, 4.3 Hz, 1H), 4.91 (d, J = 16.6 Hz, 1H), |
| | 4.80 (h, J = 6.0 Hz, 1H), 4.61 (d, J = 16.6 Hz, 1H), 4.35 (t, J = |
| | 11.3 Hz, 1H), 3.97-3.81 (m, 1H), 2.10 (s, 4H), 1.94 (s, 3H), |
| | 1.92-1.81 (m, 2H), 1.76-1.64 (m, 1H), 1.61-1.42 (m, 4H), |
| | 1.30 (dd, J = 6.0, 1.9 Hz, 6H), 0.88 (p, J = 9.1 Hz, 1H). |
| 355 | ¹H NMR (400 MHz, DMSO-d₆) δ 8.59 (s, 1H), 8.52 (s, 2H), 7.90 |
| | (s, 1H), 7.67 (s, 2H), 7.28 (t, J = 7.5 Hz, 1H), 7.16 (dd, J = 15.5, |
| | 7.7 Hz, 2H), 5.36 (dd, J = 11.0, 4.3 Hz, 1H), 4.91 (d, J = 16.5 |
| | Hz, 1H), 4.80 (h, J = 6.0 Hz, 1H), 4.59 (d, J = 16.5 Hz, 1H), |
| | 4.24 (t, J = 11.2 Hz, 1H), 3.91-3.83 (m, 1H), 2.04 (s, 3H), 1.91- |
| | 1.85 (m, 4H), 1.72-1.64 (m, 2H), 1.62 (s, 3H), 1.59-1.42 (m, |
| | 5H), 1.30 (dd, J = 6.0, 2.0 Hz, 6H), 0.89-0.75 (m, 2H). |
| 356 | ¹H NMR (500 MHz, DMSO-d₆) δ 12.90 (s, 1H), 8.70 (s, 1H), |
| | 8.08 (s, 3H), 7.92 (d, J = 7.6 Hz, 1H), 7.68-7.57 (m, 2H), 7.32 |
| | (s, 1H), 6.98 (d, J = 8.4 Hz, 1H), 6.85 (d, J = 7.6 Hz, 1H), 5.40 |
| | (d, J = 9.7 Hz, 1H), 4.88 (d, J = 16.2 Hz, 1H), 4.59 (p, J = 6.1 |
| | Hz, 1H), 4.52 (d, J = 16.2 Hz, 1H), 4.05 (d, J = 10.1 Hz, 1H), |
| | 3.39 (t, J = 7.0 Hz, 2H), 3.09 (s, 2H), 1.78 (t, J = 6.6 Hz, 5H), |
| | 1.61 (s, 3H), 1.37 (d, J = 15.1 Hz, 1H), 1.19 (d, J = 6.0 Hz, 3H), |
| | 1.15 (d, J = 6.0 Hz, 4H), 1.11 (s, 7H), 0.54 (s, 9H). |
| 357 | ¹H NMR (400 MHz, Chloroform-d) δ 8.90 (t, J = 1.9 Hz, 1H), |
| | 8.14-8.07 (m, 1H), 7.92-7.84 (m, 3H), 7.65 (t, J = 7.8 Hz, 1H), |
| | 7.25 (overlapped with CDCl₃, t, J = 7.6 Hz, 1H), 7.12 (d, J = 7.7 |
| | Hz, 1H), 7.06 (d, J = 7.5 Hz, 1H), 5.49 (dd, J = 11.0, 4.3 Hz, |
| | 1H), 5.12 (d, J = 15.5 Hz, 1H), 4.54 (p, J = 8.5 Hz, 1H), 4.30- |
| | 4.19 (m, 1H), 4.17-4.04 (m, 2H), 3.16 (s, 3H), 2.34-2.12 (m, |
| | 6H), 1.83-1.68 (m, 10H), 1.54 (d, J = 14.9 Hz, 1H), 0.84-0.77 |
| | (m, 6H), 0.59 (s, 9H). |
| 358 | ¹H NMR (400 MHz, CDCl₃) δ 8.82 (s, 1H), 8.07 (d, J = 7.7 Hz, |
| | 1H), 7.88 (s, 2H), 7.84 (d, J = 7.4 Hz, 1H), 7.63 (t, J = 7.7 Hz, |
| | 1H), 7.23 (t, J = 7.6 Hz, 1H), 7.11 (d, J = 7.6 Hz, 1H), 7.07 (d, |
| | J = 7.5 Hz, 1H), 5.54-5.40 (m, 1H), 5.16 (d, J = 13.7 Hz, 1H), |
| | 4.62-4.46 (m, 1H), 4.31-4.15 (m, 2H), 4.15-4.04 (m, 1H), |
| | 3.18 (s, 3H), 2.42-2.27 (m, 2H), 2.27-2.13 (m, 2H), 2.02 (s, |
| | 3H), 1.90 (s, 3H), 1.82-1.72 (m, 3H), 1.72 (s, 3H), 1.51-1.38 |
| | (m, 1H), 1.32 (t, J = 12.1 Hz, 1H), 0.83 (d, J = 6.4 Hz, 3H), 0.37 |
| | (d, J = 6.2 Hz, 3H). |
| 359 | ¹H NMR (400 MHz, DMSO-d₆) δ 12.98 (s, 1H), 8.70 (s, 1H), |
| | 7.99 (s, 1H), 7.95 (s, 1H), 7.88 (s, 1H), 7.76-7.59 (m, 2H), 7.28 |
| | (t, J = 7.6 Hz, 1H), 7.18 (d, J = 7.7 Hz, 1H), 7.12 (d, J = 7.5 Hz, |
| | 1H), 5.48-5.36 (m, 1H), 4.86-4.70 (m, 2H), 4.47 (d, J = 15.9 |
| | Hz, 1H), 4.35 (t, J = 11.1 Hz, 1H), 4.10-3.99 (m, 1H), 3.07 (s, |
| | 3H), 2.25-2.11 (m, 4H), 2.06 (s, 3H), 1.88-1.74 (m, 4H), 1.69- |
| | 1.56 (m, 5H), 1.44-1.35 (m, 1H), 0.54 (s, 9H). |
| 360 | ¹H NMR (400 MHz, Chloroform-d) δ 8.90 (t, J = 1.8 Hz, 1H), |
| | 8.10 (dt, J = 8.0, 1.4 Hz, 1H), 7.90 (d, J = 3.9 Hz, 2H), 7.87 (dt, |
| | J = 7.7, 1.5 Hz, 1H), 7.64 (t, J = 7.8 Hz, 1H), 7.23 (overlapped |
| | with CDCl₃, t, J = 7.6 Hz, 1H), 7.12 (d, J = 7.7 Hz, 1H), 7.05 (d, |
| | J = 7.5 Hz, 1H), 5.48 (dd, J = 11.1, 4.3 Hz, 1H), 5.13 (d, J = |
| | 15.5 Hz, 1H), 4.56 (p, J = 8.5 Hz, 1H), 4.29-4.19 (m, 1H), 4.17- |
| | 4.06 (m, 2H), 3.15 (s, 3H), 2.34-2.10 (m, 6H), 1.79 (s, 3H), |
| | 1.74-1.69 (m, 5H), 1.66-1.58 (m, 4H), 1.57-1.48 (m, 4H), |
| | 1.17-1.09 (m, 3H), 0.83-0.71 (m, 2H), 0.59 (s, 9H). |
| 361 | ¹H NMR (400 MHz, DMSO-d₆) δ 13.03 (br. s., 1H), 8.69-8.59 |
| | (m, 1H), 8.03-7.91 (m, 1H), 7.70 (br. s., 2H), 7.58 (s, 1H), 7.33- |
| | 7.21 (m, 1H), 7.20-7.06 (m, 2H), 6.00 (s, 1H), 5.28 (br. s., |
| | 1H), 4.01-3.81 (m, 2H), 3.71 (s, 3H), 2.05 (br. s., 3H), 1.96 (d, |
| | J = 1.0 Hz, 3H), 1.75 (br. s., 3H), 1.64-1.52 (m, 7H), 1.17 (d, J = |
| | 15.9 Hz, 1H), 0.49 (s, 9H). |
| 362 | ¹H NMR (400 MHz, DMSO-d₆) δ 13.15 (br. s, 1H), 8.54 (s, 2H), |
| | 8.26 (br. s, 1H), 8.04-7.89 (m, 1H), 7.83-7.75 (m, 1H), 7.74- |
| | 7.68 (m, 1H), 7.27-7.18 (m, 1H), 7.16-6.99 (m, 3H), 5.13- |
| | 5.04 (m, 1H), 4.88-4.77 (m, 2H), 4.68 (d, J = 16.4 Hz, 1H), |
| | 4.61-4.48 (m, 1H), 3.82-3.68 (m, 1H), 1.98 (br. s, 3H), 1.84 |
| | (dd, J = 15.3, 10.1 Hz, 1H), 1.74 (s, 3H), 1.41 (d, J = 14.4 Hz, |
| | 1H), 1.33-1.29 (m, 6H), 0.59 (s, 9H). |
| 363 | ¹H NMR (400 MHz, DMSO-d₆) δ 13.14 (br. s, 1H), 8.26 (br. s, |
| | 1H), 8.02 (s, 1H), 8.00-7.94 (m, 2H), 7.83-7.76 (m, 1H), 7.75- |
| | 7.69 (m, 1H), 7.26-7.19 (m, 1H), 7.14-7.09 (m, 2H), 7.09- |
| | 7.02 (m, 1H), 5.17 (dd, J = 12.2, 2.9 Hz, 1H), 4.83 (quin, J = 8.5 |
| | Hz, 1H), 4.72-4.58 (m, 2H), 4.49 (d, J = 15.7 Hz, 1H), 3.83- |
| | 3.71 (m, 1H), 3.11 (s, 3H), 2.27-2.17 (m, 4H), 1.99 (s, 3H), 1.85 |
| | (dd, J = 15.3, 10.1 Hz, 1H), 1.74 (s, 3H), 1.71-1.64 (m, 2H), |
| | 1.42 (d, J = 15.7 Hz, 1H), 0.57 (s, 9H). |
| 364 | ¹H NMR (400 MHz, DMSO-d₆) δ 13.05 (br. s., 1H), 8.67 (br. s., |
| | 1H), 7.98 (br. s., 1H), 7.81-7.62 (m, 2H), 7.51 (s, 1H), 7.33- |
| | 7.23 (m, 1H), 7.17 (d, J = 6.4 Hz, 1H), 7.12 (d, J = 7.1 Hz, 1H), |
| | 5.39 (br. s., 1H), 4.07-3.96 (m, 1H), 3.93-3.85 (m, 1H), 3.82 |
| | (s, 3H), 2.66-2.58 (m, 1H), 2.57-2.52 (m, 1H, overlapped with |
| | DMSO), 2.05 (br. s., 3H), 1.93 (br. s., 1H), 1.83-1.66 (m, 4H), |
| | 1.56 (br. s., 3H), 1.25-1.19 (m, 1H), 0.92 (d, J = 6.4 Hz, 3H), |
| | 0.86 (d, J = 6.6 Hz, 3H), 0.55 (s, 9H). |
| 365 | ¹H NMR (400 MHz, Chloroform-d) δ 8.74 (t, J = 1.9 Hz, 1H), |
| | 8.01 (d, J = 7.9 Hz, 1H), 7.95-7.87 (m, 3H), 7.69 (t, J = 7.8 Hz, |
| | 1H), 7.24 (t, J = 7.5 Hz, 1H), 7.11 (dd, J = 20.4, 7.6 Hz, 2H), |
| | 5.40 (dd, J = 11.3, 4.1 Hz, 1H), 5.16 (d, J = 15.4 Hz, 1H), 4.57 |
| | (p, J = 8.3 Hz, 1H), 4.27 (t, J = 11.5 Hz, 1H), 4.13-3.97 (m, |
| | 2H), 3.14 (s, 3H), 2.36-2.17 (m, 4H), 2.14 (s, 3H), 2.01 (s, 3H), |
| | 1.78-1.66 (m, 4H), 1.22-1.12 (m, 1H), 0.81 (s, 9H), 0.78-0.69 |
| | (m, 1H). Sulfonamide NH not visible. |
| 366 | ¹H NMR (400 MHz, Chloroform-d) δ 8.77 (t, J = 1.9 Hz, 1H), |
| | 7.97 (d, J = 8.1 Hz, 1H), 7.91-7.86 (m, 2H), 7.77 (s, 1H), 7.67 |
| | (t, J = 7.8 Hz, 1H), 7.26-7.23 (m, 1H), 7.14 (d, J = 7.6 Hz, 1H), |
| | 7.08 (d, J = 7.6 Hz, 1H), 5.49 (dd, J = 11.3, 4.1 Hz, 1H), 5.10 (d, |
| | J = 15.4 Hz, 1H), 4.30 (t, J = 11.5 Hz, 1H), 4.26-4.19 (m, 1H), |
| | 4.12-4.06 (m, 1H), 4.03 (d, J = 15.5 Hz, 1H), 3.69-3.62 (m, |
| | 1H), 3.53-3.43 (m, 1H), 2.16 (s, 3H), 2.14-2.04 (m, 3H), 2.01 |
| | (s, 3H), 1.77 (s, 1H), 1.71-1.65 (m, 2H), 1.27 (d, J = 6.3 Hz, |
| | 3H), 1.22-1.13 (m, 1H), 0.81 (s, 9H), 0.78-0.72 (m, 1H). |
| | Sulfonamide NH not visible. |
| 367 | ¹H NMR (400 MHz, Chloroform-d) δ 8.70 (t, J = 1.8 Hz, 1H), |
| | 8.03-7.96 (m, 2H), 7.92-7.87 (m, 2H), 7.68 (t, J = 7.8 Hz, 1H), |
| | 7.24 (t, J = 7.6 Hz, 1H), 7.11 (dd, J = 22.2, 7.6 Hz, 2H), 5.36 |
| | (dd, J = 11.3, 4.1 Hz, 1H), 5.13 (d, J = 15.4 Hz, 1H), 4.70-4.61 |
| | (m, 1H), 4.25 (t, J = 11.5 Hz, 2H), 4.12-4.04 (m, 1H), 4.01 (d, |
| | J = 15.5 Hz, 1H), 3.01 (td, J = 13.0, 3.2 Hz, 1H), 2.15 (s, 3H), |
| | 2.01 (s, 3H), 1.86-1.63 (m, 8H), 1.24 (d, J = 6.8 Hz, 3H), 1.16 |
| | (td, J = 12.5, 12.1, 5.6 Hz, 1H), 0.81 (s, 9H), 0.78-0.70 (m, |
| | 1H). Sulfonamide NH not visible. |
| 368 | ¹H NMR (400 MHz, Chloroform-d) δ 8.85 (d, J = 2.3 Hz, 1H), |
| | 8.52 (s, 1H), 7.98 (d, J = 8.0 Hz, 1H), 7.90 (d, J = 8.0 Hz, 1H), |
| | 7.68 (t, J = 7.8 Hz, 1H), 7.26-7.23 (m, 1H), 7.13 (d, J = 7.6 Hz, |
| | 1H), 7.09 (d, J = 7.6 Hz, 1H), 6.54 (s, 1H), 5.52 (dd, J = 11.2, |
| | 4.0 Hz, 1H), 5.44 (d, J = 15.5 Hz, 1H), 4.36-4.28 (m, 2H), 4.12- |
| | 4.02 (m, 1H), 3.91 (s, 3H), 3.18 (p, J = 6.8 Hz, 1H), 2.13 (s, |
| | 3H), 2.03 (s, 3H), 1.81-1.71 (m, 2H), 1.39 (dd, J = 8.4, 6.8 Hz, |
| | 6H), 1.28-1.21 (m, 1H), 0.84 (s, 9H), 0.82-0.77 (m, 1H). |
| | Sulfonamide NH not visible |
| 369 | ¹H NMR (400 MHz, Chloroform-d) δ 8.86 (t, J = 1.8 Hz, 1H), |
| | 8.38 (s, 2H), 7.89-7.82 (m, 2H), 7.64 (t, J = 7.8 Hz, 1H), 7.26- |
| | 7.24 (m, 1H), 7.14 (d, J = 7.6 Hz, 1H), 7.08 (d, J = 7.6 Hz, 1H), |
| | 5.50 (d, J = 7.2 Hz, 1H), 5.38 (d, J = 16.5 Hz, 1H), 4.61 (p, J = |
| | 6.0 Hz, 1H), 4.27 (d, J = 16.6 Hz, 1H), 4.07-3.93 (m, 2H), 2.12 |
| | (s, 3H), 2.03 (s, 3H), 1.74-1.65 (m, 2H), 1.38 (dd, J = 6.1, 3.7 |
| | Hz, 6H), 1.27-1.17 (m, 1H), 0.82 (s, 9H), 0.80-0.72 (m, 1H). |
| | Sulfonamide -NH not visible. |

### D. Biological Activity

### 1. Assay Procedures

### (a) HBE Assay

### (1) Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂), 10 Glucose, 10 HEPES (pH adjusted to 7.4 with NaOH) and the apical solution contained (in mM) 145 NaGluconate, 1.2 MgCl₂, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

### (2) Identification of Corrector Compounds

The activity of the CFTR corrector compounds on the CFTR-mediated I_{SC} was determined in Ussing chamber studies as described above. The F508del/MF-HBE cell cultures were either incubated with the corrector compounds at a range of concentrations in combination with 1 μM Ivacaftor or were incubated with the corrector compounds at a single fixed concentration of 10 μM in combination with 1 μM Ivacaftor for 18-24 hours at 37° C. and in the presence of 20% human serum. The concentration of corrector compounds with 1 μM Ivacaftor during the 18-24 hours incubations was kept constant throughout the Ussing chamber measurement of the CFTR-mediated I_{SC} to ensure compounds were present throughout the entire experiment. The efficacy and potency of the putative F508del correctors was compared to that of the known Vertex corrector, (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, in combination with 18 μM Tezacaftor and 1 μM Ivacaftor.

### (b) HBE2 Assay

### (1) Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂), 10 Glucose, 10 HEPES (pH adjusted to 7.4 with NaOH) and the apical solution contained (in mM) 145 NaGluconate, 1.2 MgCl₂, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

### (2) Identification of Corrector Compounds

The activity of the CFTR corrector compounds on the CFTR-mediated I_{SC} was determined in Ussing chamber studies as described above. The F508del/MF-HBE cell cultures were either incubated with the corrector compounds at a range of concentrations in combination with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol or were incubated with the corrector compounds at a single fixed concentration of 1 and 3 μM in combination with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol for 18-24 hours at 37° C. and in the presence of 20% human serum. The concentration of corrector compounds with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol during the 18-24 hours incubations was kept constant throughout the Ussing chamber measurement of the CFTR-mediated I_{SC} to ensure compounds were present throughout the entire experiment. The efficacy and potency of the putative F508del correctors was compared to that of the known Vertex corrector, (14S′)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, in combination with 18 M Tezacaftor and 1 μM Ivacaftor.

### 2. Biological Activity Data Table

Table 24 represents CFTR modulating activity for representative compounds of the invention generated using one or more of the assays disclosed herein (EC₅₀: +++ is <1 μM; ++ is 1-<3 μM; + is 3-<30 μM; and ND is “not detected in this assay.” 0% Activity: +++ is >60%; ++ is 30-60%; + is <30%.

TABLE 24
Biological Assay Data for Compounds 331-369

| | | | HBE | HBE | HBE2 |
|---|---|---|---|---|---|
| | | HBE | Max | Activity | Activity |
| Compound | | EC₅₀ | activity | at 10 | at 3 μM |
| No. | Structure | (μM) | (%) | μM (%) | (%) |
| 331 | 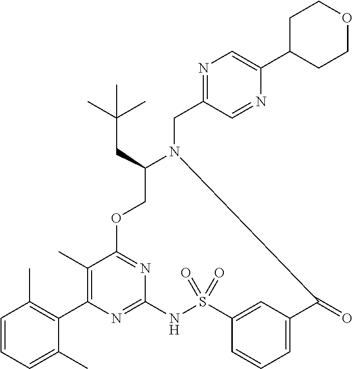 [SMILES: Cc1cccc(C)c1-c1nc2nc(c1C)OC[C@@H](CC(C)(C)C)N(Cc1cnc(C3CCOCC3)cn1)C(=O)c1cccc(c1)S(=O)(=O)N2] | +++ | +++ | | |
| 332 | 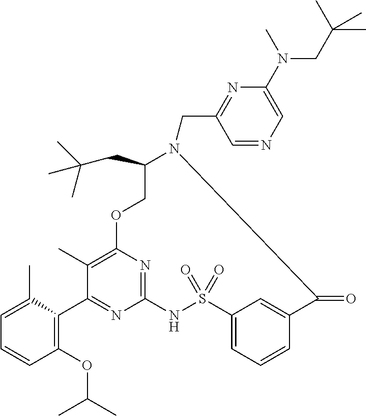 | +++ | +++ | | |
| 333 | 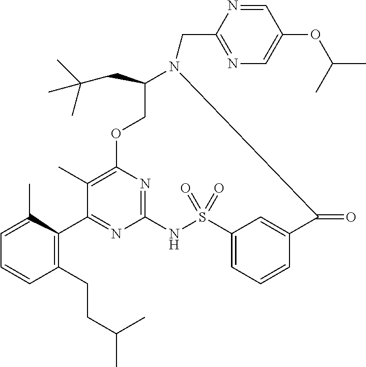 | | | | +++ |
| 334 | 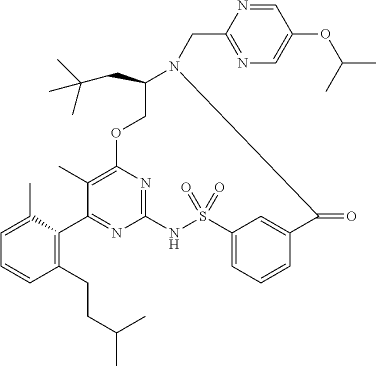 | | | | +++ |
| 335 | 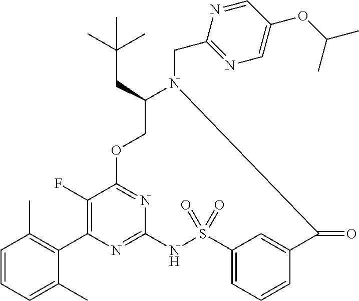 [SMILES: Cc1cccc(C)c1-c1nc2nc(c1F)OC[C@@H](CC(C)(C)C)N(Cc1ncc(OC(C)C)cn1)C(=O)c1cccc(c1)S(=O)(=O)N2] | +++ | +++ | | |
| 336 | 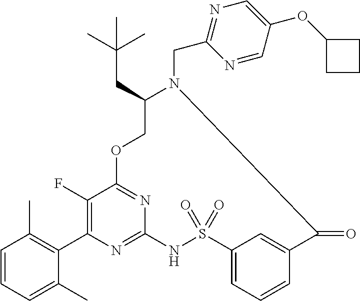 [SMILES: Cc1cccc(C)c1-c1nc2nc(c1F)OC[C@@H](CC(C)(C)C)N(Cc1ncc(OC3CCC3)cn1)C(=O)c1cccc(c1)S(=O)(=O)N2] | +++ | +++ | | |
| 337 | 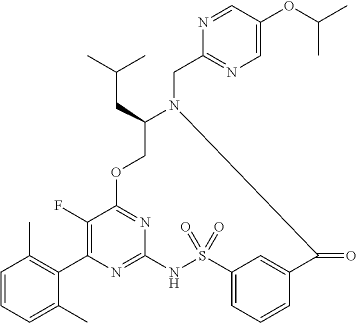 [SMILES: Cc1cccc(C)c1-c1nc2nc(c1F)OC[C@@H](CC(C)C)N(Cc1ncc(OC(C)C)cn1)C(=O)c1cccc(c1)S(=O)(=O)N2] | +++ | +++ | | |
| 338 | 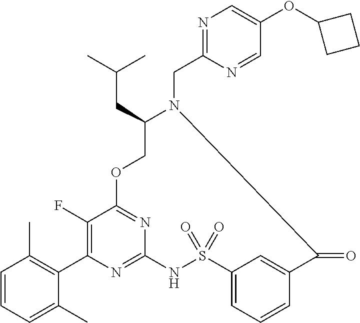 [SMILES: Cc1cccc(C)c1-c1nc2nc(c1F)OC[C@@H](CC(C)C)N(Cc1ncc(OC3CCC3)cn1)C(=O)c1cccc(c1)S(=O)(=O)N2] | +++ | +++ | | |
| 339 | 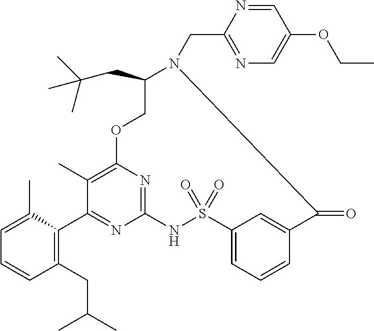 | +++ | +++ | | |
| 340 | 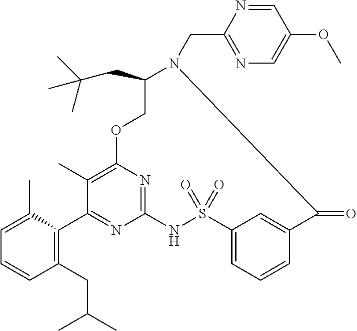 | | | | |
| 341 | 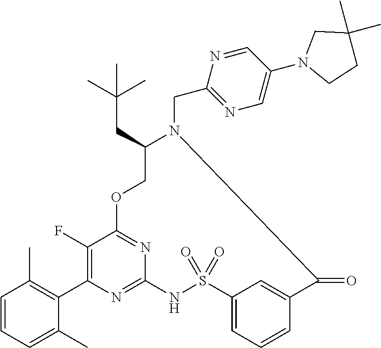 [SMILES: Cc1cccc(C)c1-c1nc2nc(c1F)OC[C@@H](CC(C)(C)C)N(Cc1ncc(N3CCC(C)(C)C3)cn1)C(=O)c1cccc(c1)S(=O)(=O)N2] | +++ | +++ | | |
| 342 | 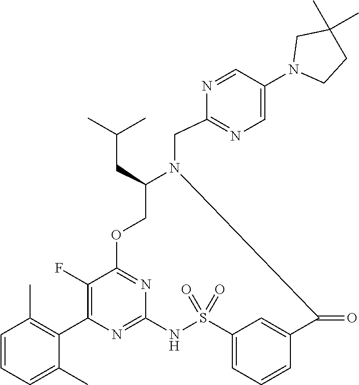 [SMILES: Cc1cccc(C)c1-c1nc2nc(c1F)OC[C@@H](CC(C)C)N(Cc1ncc(N3CCC(C)(C)C3)cn1)C(=O)c1cccc(c1)S(=O)(=O)N2] | +++ | +++ | | |
| 343 | 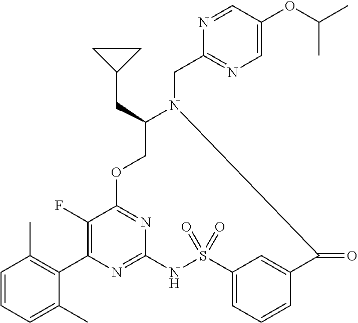 [SMILES: Cc1cccc(C)c1-c1nc2nc(c1F)OC[C@@H](CC1CC1)N(Cc1ncc(OC(C)C)cn1)C(=O)c1cccc(c1)S(=O)(=O)N2] | ++ | +++ | | |
| 344 | 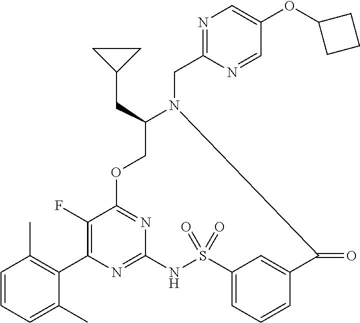 [SMILES: Cc1cccc(C)c1-c1nc2nc(c1F)OC[C@@H](CC1CC1)N(Cc1ncc(OC3CCC3)cn1)C(=O)c1cccc(c1)S(=O)(=O)N2] | ++ | +++ | | |
| 345 | 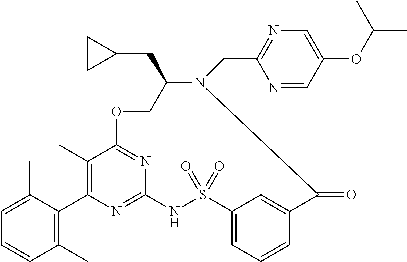 [SMILES: Cc1cccc(C)c1-c1nc2nc(c1C)OC[C@@H](CC1CC1)N(Cc1ncc(OC(C)C)cn1)C(=O)c1cccc(c1)S(=O)(=O)N2] | +++ | +++ | | |
| 346 | 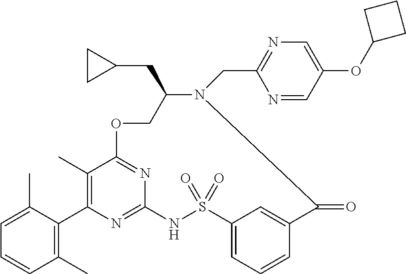 [SMILES: Cc1cccc(C)c1-c1nc2nc(c1C)OC[C@@H](CC1CC1)N(Cc1ncc(OC3CCC3)cn1)C(=O)c1cccc(c1)S(=O)(=O)N2] | +++ | +++ | | |
| 347 | 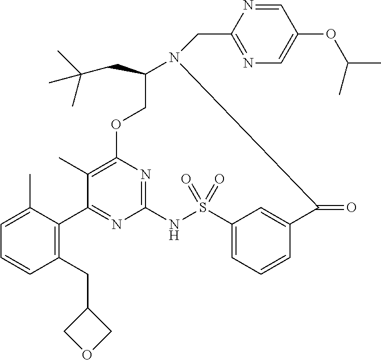 [SMILES: Cc1cccc(CC2COC2)c1-c1nc2nc(c1C)OC[C@@H](CC(C)(C)C)N(Cc1ncc(OC(C)C)cn1)C(=O)c1cccc(c1)S(=O)(=O)N2] | | | +++ | |
| 348 | 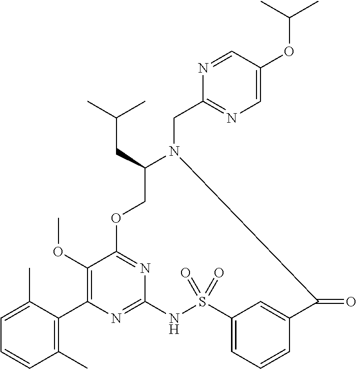 [SMILES: COc1c2nc(nc1-c1c(C)cccc1C)NS(=O)(=O)c1cccc(c1)C(=O)N(Cc1ncc(OC(C)C)cn1)[C@H](CC(C)C)CO2] | +++ | +++ | | |
| 349 | 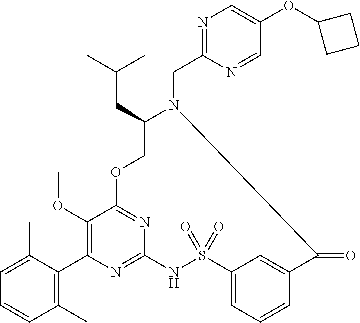 [SMILES: COc1c2nc(nc1-c1c(C)cccc1C)NS(=O)(=O)c1cccc(c1)C(=O)N(Cc1ncc(OC3CCC3)cn1)[C@H](CC(C)C)CO2] | +++ | +++ | | |
| 350 | 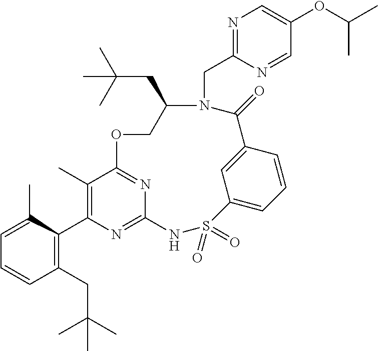 | | | | |
| 351 | 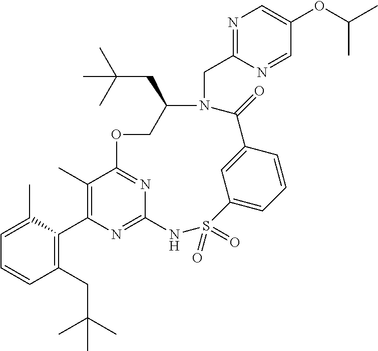 | +++ | +++ | +++ | |
| 352 | 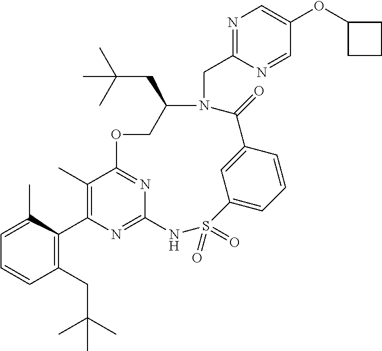 | | | | |
| 353 | 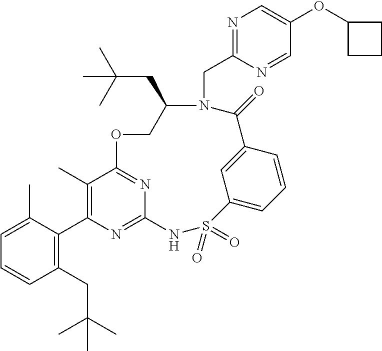 [SMILES: Cc1cccc(CC(C)(C)C)c1-c1nc2nc(c1C)OC[C@@H](CC(C)(C)C)N(Cc1ncc(OC3CCC3)cn1)C(=O)c1cccc(c1)S(=O)(=O)N2] | | | +++ | |
| 354 | 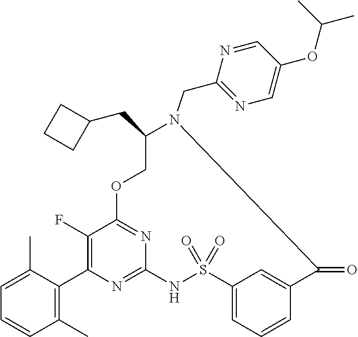 [SMILES: Cc1cccc(C)c1-c1nc2nc(c1F)OC[C@@H](CC1CCC1)N(Cc1ncc(OC(C)C)cn1)C(=O)c1cccc(c1)S(=O)(=O)N2] | | | +++ | |
| 355 | 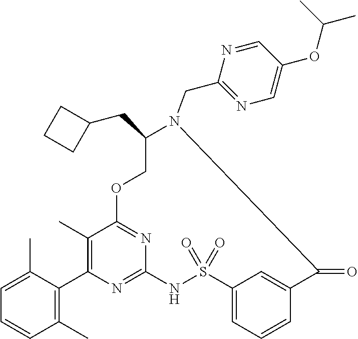 [SMILES: Cc1cccc(C)c1-c1nc2nc(c1C)OC[C@@H](CC1CCC1)N(Cc1ncc(OC(C)C)cn1)C(=O)c1cccc(c1)S(=O)(=O)N2] | | | +++ | |
| 356 | 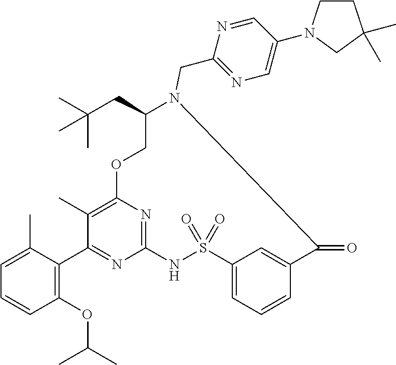 [SMILES: Cc1cccc(OC(C)C)c1-c1nc2nc(c1C)OC[C@@H](CC(C)(C)C)N(Cc1ncc(N3CCC(C)(C)C3)cn1)C(=O)c1cccc(c1)S(=O)(=O)N2] | | | | +++ |
| 357 | 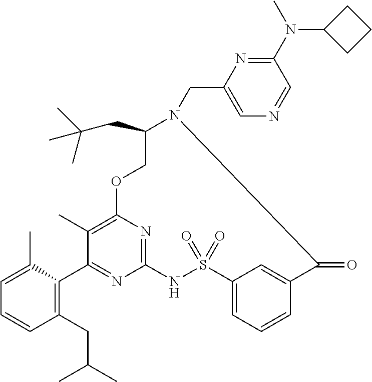 | | | | +++ |
| 358 | 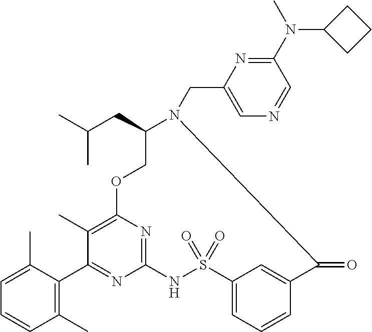 [SMILES: Cc1cccc(C)c1-c1nc2nc(c1C)OC[C@@H](CC(C)C)N(Cc1cncc(N(C)C3CCC3)n1)C(=O)c1cccc(c1)S(=O)(=O)N2] | | | | +++ |
| 359 | 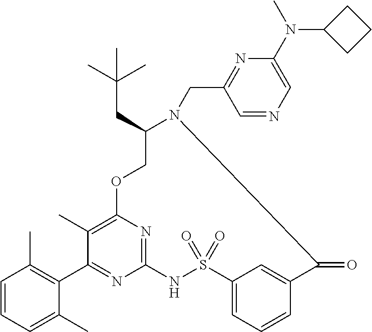 [SMILES: Cc1cccc(C)c1-c1nc2nc(c1C)OC[C@@H](CC(C)(C)C)N(Cc1cncc(N(C)C3CCC3)n1)C(=O)c1cccc(c1)S(=O)(=O)N2] | | | | +++ |
| 360 | 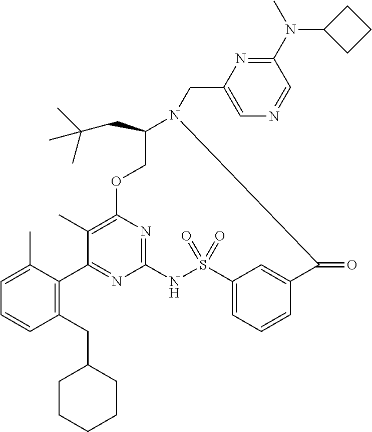 [SMILES: Cc1cccc(CC2CCCCC2)c1-c1nc2nc(c1C)OC[C@@H](CC(C)(C)C)N(Cc1cncc(N(C)C3CCC3)n1)C(=O)c1cccc(c1)S(=O)(=O)N2] | | | | +++ |
| 361 | 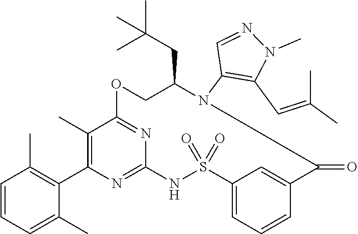 [SMILES: CC(C)=Cc1c(N2C(=O)c3cccc(c3)S(=O)(=O)Nc3nc(c(C)c(-c4c(C)cccc4C)n3)OC[C@H]2CC(C)(C)C)cnn1C] | | | | +++ |
| 362 | 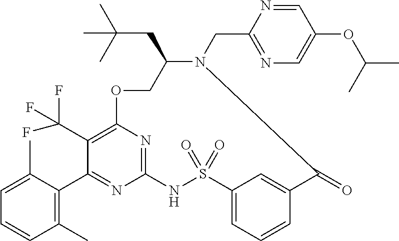 [SMILES: Cc1cccc(C)c1-c1nc2nc(c1C(F)(F)F)OC[C@@H](CC(C)(C)C)N(Cc1ncc(OC(C)C)cn1)C(=O)c1cccc(c1)S(=O)(=O)N2] | | | | +++ |
| 363 | 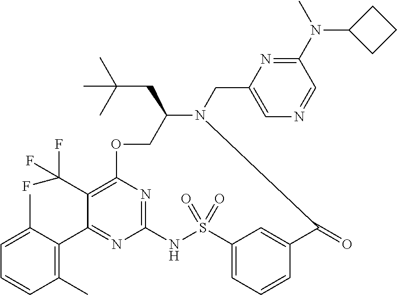 [SMILES: Cc1cccc(C)c1-c1nc2nc(c1C(F)(F)F)OC[C@@H](CC(C)(C)C)N(Cc1cncc(N(C)C3CCC3)n1)C(=O)c1cccc(c1)S(=O)(=O)N2] | | | | +++ |
| 364 | 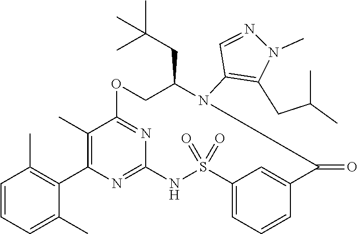 [SMILES: Cc1cccc(C)c1-c1nc2nc(c1C)OC[C@@H](CC(C)(C)C)N(c1cnn(C)c1CC(C)C)C(=O)c1cccc(c1)S(=O)(=O)N2] | | | | +++ |
| 365 | 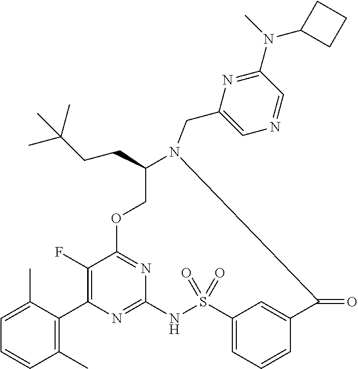 [SMILES: Cc1cccc(C)c1-c1nc2nc(c1F)OC[C@@H](CCC(C)(C)C)N(Cc1cncc(N(C)C3CCC3)n1)C(=O)c1cccc(c1)S(=O)(=O)N2] | | | | +++ |
| 366 | 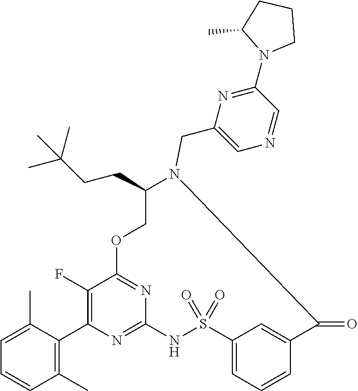 [SMILES: Cc1cccc(C)c1-c1nc2nc(c1F)OC[C@@H](CCC(C)(C)C)N(Cc1cncc(N3CCC[C@H]3C)n1)C(=O)c1cccc(c1)S(=O)(=O)N2] | | | | +++ |
| 367 | 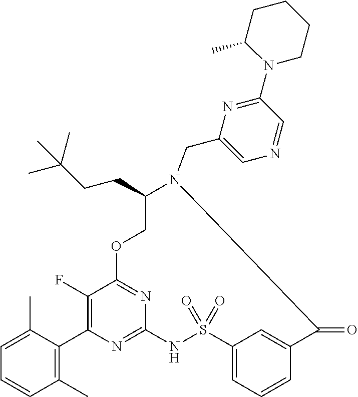 [SMILES: Cc1cccc(C)c1-c1nc2nc(c1F)OC[C@@H](CCC(C)(C)C)N(Cc1cncc(N3CCCC[C@H]3C)n1)C(=O)c1cccc(c1)S(=O)(=O)N2] | | | | +++ |
| 368 | 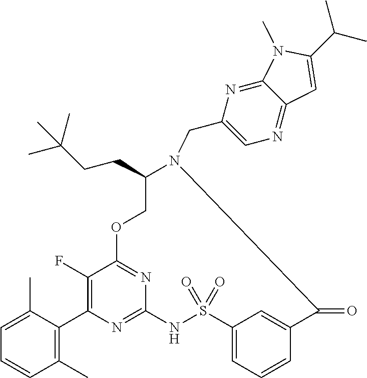 [SMILES: Cc1cccc(C)c1-c1nc2nc(c1F)OC[C@@H](CCC(C)(C)C)N(Cc1cnc3cc(C(C)C)n(C)c3n1)C(=O)c1cccc(c1)S(=O)(=O)N2] | | | | +++ |
| 369 | 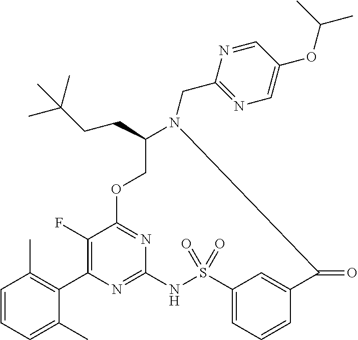 [SMILES: Cc1cccc(C)c1-c1nc2nc(c1F)OC[C@@H](CCC(C)(C)C)N(Cc1ncc(OC(C)C)cn1)C(=O)c1cccc(c1)S(=O)(=O)N2] | | | | +++ |

### VIII. Synthesis of (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol

### A. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32k points and a line broadening of 0.3 Hz was applied before Fourier transform. ¹⁹F NMR spectra were recorded at 282 MHz using a 30 degree pulse angle, a spectral width of 100 kHz and 59202 points were acquired. FID were zero-filled to 64k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128k points of acquisition. FID were zero-filled to 256k points and a line broadening of 0.3 Hz was applied before fourrier transform. ¹⁹F NMR spectra were recorded at 377 MHz using a 30 deg pulse angle, a spectral width of 89286 Hz and 128k points were acquired. FID were zero-filled to 256k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Unless stated to the contrary in the following examples, final purity of compounds was determined by reversed phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range.

Solid-state NMR (SSNMR) spectra were recorded on a Bruker-Biospin 400 MHz wide-bore spectrometer equipped with Bruker-Biospin 4 mm HFX probe. Samples were packed into 4 mm ZrO2 rotors and spun under Magic Angle Spinning (MAS) condition with spinning speed typically set to 12.5 kHz. The proton relaxation time was measured using ¹H MAS Ti saturation recovery relaxation experiment in order to set up proper recycle delay of the ¹³C cross-polarization (CP) MAS experiment. The fluorine relaxation time was measured using ¹⁹F MAS Ti saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

### B. Procedures for the Synthesis of Intermediates

### Intermediate 1: Preparation of methyl 3-[bis(tert-butoxycarbonyl)amino]-6-bromo-5-(trifluoromethyl)pyridine-2-carboxylate

### Step 1: Methyl 3-(benzhydrylideneamino)-5-(trifluoromethyl)pyridine-2-carboxylate

A mixture of methyl 3-chloro-5-(trifluoromethyl)pyridine-2-carboxylate (47.3 g, 197.43 mmol), diphenylmethanimine (47 g, 259.33 mmol), Xantphos (9.07 g, 15.675 mmol), and cesium carbonate (131 g, 402.06 mmol) in dioxane (800 mL) was degassed with bubbling nitrogen for 30 minutes. Pd(OAc)₂ (3.52 g, 15.679 mmol) was added and the system was purged with nitrogen three times. The reaction mixture was heated at 100° C. for 18 h. The reaction was cooled to room temperature and filtered on a pad of Celite. The cake was washed with EtOAc and solvents were evaporated under reduced pressure to give methyl 3-(benzhydrylideneamino)-5-(trifluoromethyl)pyridine-2-carboxylate (90 g, 84%) as yellow solid. ESI-MS m/z calc. 384.10855, found 385.1 (M+1)⁺; Retention time: 2.24 minutes. LCMS Method: Kinetex C₁₈ 4.6×50 mm 2.6 μM, 2.0 mL/min, 95% H₂O (0.1% formic acid)+5% acetonitrile (0.1% formic acid) to 95% acetonitrile (0.1% formic acid) gradient (2.0 min) then held at 95% acetonitrile (0.1% formic acid) for 1.0 min.

### Step 2: Methyl 3-amino-5-(trifluoromethyl)pyridine-2-carboxylate

To a suspension of methyl 3-(benzhydrylideneamino)-5-(trifluoromethyl)pyridine-2-carboxylate (65 g, 124.30 mmol) in methanol (200 mL) was added HCl (3 M in methanol) (146 mL of 3 M, 438.00 mmol). The mixture was stirred at room temperature for 1.5 hour then the solvent was removed under reduced pressure. The residue was taken up in ethyl acetate (2 L) and dichloromethane (500 mL). The organic phase was washed with 5% aqueous sodium bicarbonate solution (3×500 mL) and brine (2×500 mL), dried over anhydrous sodium sulfate, filtered and the solvent was removed under reduced pressure. The residue was triturated with heptanes (2×50 mL) and the mother liquors were discarded. The solid obtained was triturated with a mixture of dichloromethane and heptanes (1:1, 40 mL) and filtered to afford methyl 3-amino-5-(trifluoromethyl)pyridine-2-carboxylate (25.25 g, 91%) as yellow solid. ¹H NMR (300 MHz, CDCl₃) δ 8.24 (s, 1H), 7.28 (s, 1H), 5.98 (br. s, 2H), 4.00 (s, 3H) ppm. 19F NMR (282 MHz, CDCl₃) δ −63.23 (s, 3F) ppm. ESI-MS m/z calc. 220.046, found 221.1 (M+1)⁺; Retention time: 1.62 minutes. LCMS Method: Kinetex Polar C₁₈ 3.0×50 mm 2.6 μm, 3 min, 5-95% acetonitrile in H₂O (0.1% formic acid) 1.2 mL/min.

### Step 3: Methyl 3-amino-6-bromo-5-(trifluoromethyl)pyridine-2-carboxylate

To a solution of methyl 3-amino-5-(trifluoromethyl)pyridine-2-carboxylate (18.75 g, 80.91 mmol) in acetonitrile (300 mL) at 0° C. was added portion wise N-bromosuccinimide (18.7 g, 105.3 mmol). The mixture was stirred overnight at 25° C. Ethyl acetate (1000 mL) was added. The organic layer was washed with 10% sodium thiosulfate solution (3×200 mL) which were back extracted with ethyl acetate (2×200 mL). The combined organic extracts were washed with saturated sodium bicarbonate solution (3×200 mL), brine (200 mL), dried over sodium sulfate and concentrated in vacuo to provide methyl 3-amino-6-bromo-5-(trifluoromethyl)pyridine-2-carboxylate (25.46 g, 98%). ¹H NMR (300 MHz, CDCl₃) δ 3.93-4.03 (m, 3H), 6.01 (br. s., 2H), 7.37 (s, 1H) ppm. ¹⁹F NMR (282 MHz, CDCl₃) ppm −64.2 (s, 3F). ESI-MS m/z calc. 297.9565, found 299.0 (M+1)⁺; Retention time: 2.55 minutes. LCMS Method: Kinetex C₁₈ 4.6×50 mm 2.6 μM. Temp: 45° C., Flow: 2.0 mL/min, Run Time: 6 min. Mobile Phase: Initial 95% H₂O (0.1% formic acid) and 5% acetonitrile (0.1% formic acid) linear gradient to 95% acetonitrile (0.1% formic acid) for 4.0 min then held at 95% acetonitrile (0.1% formic acid) for 2.0 min.

### Step 4: Methyl 3-[bis(tert-butoxycarbonyl)amino]-6-bromo-5-(trifluoro methyl)pyridine-2-carboxylate

A mixture of methyl 3-amino-6-bromo-5-(trifluoromethyl)pyridine-2-carboxylate (5 g, 15.549 mmol), (Boc)₂O (11 g, 11.579 mL, 50.402 mmol), DMAP (310 mg, 2.5375 mmol) and CH₂Cl₂ (150 mL) was stirred at room temperature overnight. The reaction mixture was concentrated under reduced pressure and purification by silica gel chromatography (0-15% ethyl acetate in heptane) provided methyl 3-[bis(tert-butoxycarbonyl)amino]-6-bromo-5-(trifluoromethyl)pyridine-2-carboxylate (6.73 g, 87%) as light yellow solid. ¹H NMR (300 MHz, CDCl₃) δ 1.42 (s, 18H), 3.96 (s, 3H), 7.85 (s, 1H) ppm. ¹⁹F NMR (282 MHz, CDCl₃) δ −63.9 (s, 3F) ppm. ESI-MS m/z calc. 498.06134, Retention time: 2.34 minutes. LCMS Method: Kinetex C₁₈ 4.6×50 mm 2.6 μM. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Intermediate 2: Preparation of 6-bromo-3-(tert-butoxycarbonylamino)-5-(trifluoromethyl)pyridine-2-carboxylic acid

### Step 1: 6-Bromo-3-(tert-butoxycarbonylamino)-5-(trifluoromethyl)pyridine-2-carboxylic acid

To a mixture of methyl 3-[bis(tert-butoxycarbonyl)amino]-6-bromo-5-(trifluoromethyl)pyridine-2-carboxylate (247 g, 494.7 mmol) in THF (1.0 L) was added a solution of LiOH (47.2 g, 1.971 mol) in water (500 mL). The mixture was stirred at ambient temperature for 18 h affording a yellow slurry. The mixture was cooled with an ice-bath and slowly acidified with HCl (1000 mL of 2 M, 2.000 mol) keeping the reaction temperature <15° C. The mixture was diluted with heptane (1.5 L), mixed and the organic phase separated. The aqueous phase was extracted with heptane (500 mL). The combined organic phases were washed with brine, dried over MgSO₄, filtered and concentrated in vacuo. The crude oil was dissolved in heptane (600 mL), seeded and stirred at ambient temperature for 18 h affording a thick slurry. The slurry was diluted with cold heptane (500 mL) and the precipitate collected using a medium frit. The filter cake was washed with cold heptane and air dried for 1 h, then in vacuo at 45° C. for 48 h to afford 6-bromo-3-(tert-butoxycarbonylamino)-5-(trifluoromethyl)pyridine-2-carboxylic acid (158.3 g, 83%). ¹H NMR (400 MHz, DMSO-d₆) δ 10.38 (s, 1H), 9.01 (s, 1H), 1.50 (s, 9H) ppm. ESI-MS m/z calc. 383.99326, found 384.9 (M+1)⁺; Retention time: 2.55 minutes. LCMS Method Detail: Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=acetonitrile (0.035% CF₃CO₂H). Flow rate=1.2 mL/min, injection volume=1.5 μL, and column temperature=60° C.

### Intermediate 3: Preparation of 2-Benzyloxy-2-(trifluoromethyl)hex-5-enoic acid

### Step 1: Ethyl 2-hydroxy-2-(trifluoromethyl)hex-5-enoate

To a solution of ethyl 3,3,3-trifluoro-2-oxo-propanoate (25.15 g, 147.87 mmol) in Et₂₀ (270 mL) at −78° C. was added bromo(but-3-enyl)magnesium in THF (190 mL of 0.817 M, 155.23 mmol) dropwise over a period of 1.5 h (inner temperature −72° C. to −76° C.). The mixture was stirred at −78° C. for 20 min. The dry ice-acetone bath was removed. The mixture was slowly warm to 5° C. during 1 h, added to a mixture of 1 N aqueous HCl (170 mL) and crushed ice (150 g) (pH=4). The two layers were separated. The organic layer was concentrated, and the residue was combined with aqueous phase and extracted with EtOAc (2×150 mL). The combined organic phase was washed with 5% aqueous NaHCO₃(50 mL) and brine (20 mL), dried with Na₂SO₄. The mixture was filtered and concentrated, and co-evaporated with THF (2×40 mL) to give ethyl 2-hydroxy-2-(trifluoromethyl)hex-5-enoate (37.44 g, 96%) as colorless oil. ¹H NMR (300 MHz, CDCl₃) δ 5.77 (ddt, J=17.0, 10.4, 6.4 Hz, 1H), 5.15-4.93 (m, 2H), 4.49-4.28 (m, 2H), 3.88 (s, 1H), 2.35-2.19 (m, 1H), 2.17-1.89 (m, 3H), 1.34 (t, J=7.0 Hz, 3H) ppm. ¹⁹F NMR (282 MHz, CDCl₃) δ −78.74 (s, 3F) ppm.

### Step 2: Ethyl 2-benzyloxy-2-(trifluoromethyl)hex-5-enoate

To a solution of ethyl 2-hydroxy-2-(trifluoromethyl)hex-5-enoate (24.29 g, 87.6% purity, 94.070 mmol) in DMF (120 mL) at 0° C. was added NaH (60% in mineral oil, 5.64 g, 141.01 mmol) portion-wise. The mixture was stirred at 0° C. for 10 min. Benzyl bromide (24.13 g, 141.08 mmol) and TBAI (8.68 g, 23.500 mmol) were added. The mixture was stirred at room temperature overnight. NH₄Cl (3 g, 0.6 eq) was added. The mixture was stirred for 10 min. 30 mL of EtOAc was added, then ice-water was added (400 g). The mixture was extracted with CH₂C₁₂ and the combined organic layers were concentrated. Purification by silica gel chromatography (0-20% CH₂C₁₂ in heptanes) provided ethyl 2-benzyloxy-2-(trifluoromethyl)hex-5-enoate (26.05 g, 88%) as pink oil. ¹H NMR (300 MHz, CDCl₃) δ 1.34 (t, J=7.2 Hz, 3H), 2.00-2.19 (m, 3H), 2.22-2.38 (m, 1H), 4.33 (q, J=7.2 Hz, 2H), 4.64 (d, J=10.6 Hz, 1H), 4.84 (d, J=10.9 Hz, 1H), 4.91-5.11 (m, 2H), 5.62-5.90 (m, 1H), 7.36 (s, 5H) ppm. 19F NMR (282 MHz, CDCl₃) δ −70.5 (s, 3F) ppm. ESI-MS m/z calc. 316.12863, found 317.1 (M+1)⁺; Retention time: 2.47 minutes. LCMS Method: Kinetex C₁₈ 4.6×50 mm 2.6 μM. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Step 3: 2-Benzyloxy-2-(trifluoromethyl)hex-5-enoic acid

A solution of sodium hydroxide (7.86 g, 196.51 mmol) in water (60 mL) was added to a solution of ethyl 2-benzyloxy-2-(trifluoromethyl)hex-5-enoate (24.86 g, 78.593 mmol) in methanol (210 mL). The reaction was heated at 50° C. overnight. The reaction was concentrated to remove methanol, diluted with water (150 mL) and the carboxylate sodium salt was washed with heptane (1×100 mL). The aqueous solution was acidified to pH=2 with aqueous 3N solution of HCl. The carboxylic acid was extracted with dichloromethane (3×100 mL) and dried over sodium sulfate. The solution was filtered and concentrated to give 2-benzyloxy-2-(trifluoromethyl)hex-5-enoic acid (22.57 g, 97%) as pale yellow oil. ¹H NMR (300 MHz, DMSO-d₆) δ 14.31 (br. s., 1H), 7.55-7.20 (m, 5H), 5.93-5.70 (m, 1H), 5.17-4.91 (m, 2H), 4.85-4.68 (m, 1H), 4.67-4.55 (m, 1H), 2.32-1.94 (m, 4H) ppm. ¹⁹F NMR (282 MHz, DMSO-d₆) δ −70.29 (s, 3F) ppm. ESI-MS m/z calc. 288.09732, found 287.1 (M−1); Retention time: 3.1 minutes. LCMS Method: Kinetex Polar C₁₈ 3.0×50 mm 2.6 μm, 6 min, 5-95% acetonitrile in H₂O (0.1% formic acid) 1.2 mL/min.

### Intermediate 4: Preparation of (2R)-2-benzyloxy-2-(trifluoromethyl)hex-5-enoic acid

### Step-1: (2R)-2-Benzyloxy-2-(trifluoromethyl)hex-5-enoic acid; (R)-4-quinolyl-[(2S,4S)-5-vinylquinuclidin-2-yl]methanol

To a N₂ purged jacketed reactor set to 20° C. was added isopropyl acetate (IPAC, 100 L, 0.173 M, 20 Vols), followed by previously melted 2-benzyloxy-2-(trifluoromethyl)hex-5-enoic acid (5.00 kg, 17.345 mol) and cinchonidine (2.553 kg, 8.67 mol) made into a slurry with minor amount of the reaction solvent. The reactor was set to ramp internal temperature to 80° C. over 1 hour, with solids going in solution upon heating to set temperature, then the solution was held at temperature for at least 10 minutes, then cooled to 70° C. held and seeded with chiral salt (50 g, 1.0% by wt). The mixture was stirred for 10 minutes, then ramped to 20° C. internal temperature over 4 hours, then held overnight at 20° C. The mixture was filtered, cake washed with isopropyl acetate (10.0 L, 2.0 vols) and dried under vacuum. The cake was then dried in vacuo (50° C., vacuum) to afford 4.7 kg of salt. The resulting solid salt was returned to the reactor by making a slurry with a portion of isopropyl acetate (94 L, 20 vol based on current salt wt), and pumped into reactor and stirred. The mixture was then heated to 80° C. internal, stirred hot slurry for at least 10 minutes, then ramped to 20° C. over 4-6 h, then stirred overnight at 20° C. The material was then filtered and cake washed with isopropyl acetate (9.4 L, 2.0 vol), pulled dry, cake scooped out and dried in vacuo (50° C., vacuum) to afford 3.1 kg of solid. The solid (3.1 kg) and isopropyl acetate (62 L, 20 vol based on salt solid wt) was slurried and added to a reactor, stirred under N₂ purge and heated to 80° C. and held at temperature at least 10 minutes, then ramped to 20° C. over 4-6 hours, then stirred overnight. The mixture was filtered, cake washed with isopropyl acetate (6.2 L, 2 vol), pulled dry, scooped out and dried in vacuo (50° C., vac) to afford 2.25 kg of solid salt. The solid (2.25 kg) and isopropyl acetate (45 L, 20 vol based on salt solid wt) was slurried and added to a reactor, stirred under N₂ purge and heated to 80° C., held at temperature at least 10 minutes, then ramped to 20° C. over 4-6 hours, then stirred overnight. The mixture was filtered, cake washed with isopropyl acetate (4.5 L, 2 vol), pulled dry, scooped out and dried in vacuo (50° C. to afford (2R)-2-benzyloxy-2-(trifluoromethyl)hex-5-enoic acid; (R)-4-quinolyl-[(2S,4S)-5-vinylquinuclidin-2-yl]methanol (1.886 kg, >98.0% ee) as off-white to tan solid. Chiral purity was determined by Agilent 1200 HPLC instrument using Phenomenex Lux i-Amylose-3 column (3 μm, 150×4.6 mm) and a dual, isocratic gradient run 30% to 70% mobile phase B over 20.0 minutes. Mobile phase A=H₂O (0.1% CF₃CO₂H). Mobile phase B=MeOH (0.1% CF₃CO₂H). Flow rate=1.0 mL/min, injection volume=2 μL, and column temperature=30° C., sample concentration: 1 mg/mL in 60% acetonitrile/40% water.

### Step 2: (2R)-2-Benzyloxy-2-(trifluoromethyl)hex-5-enoic acid

A suspension of (2R)-2-benzyloxy-2-(trifluoromethyl)hex-5-enoic acid; (R)-4-quinolyl-[(2S,4S)-5-vinylquinuclidin-2-yl]methanol (50 g, 87.931 mmol) in ethyl acetate (500.00 mL) was treated with an aqueous solution of hydrochloric acid (200 mL of 1 M, 200.00 mmol). After stirring 15 minutes at room temperature, the two phases were separated. The aqueous phase was extracted twice with ethyl acetate (200 mL). The combined organic layer was washed with 1 N HCl (100 mL). The organic layer was dried over sodium sulfate, filtered and concentrated. The material was dried over high vacuum overnight to give (2R)-2-benzyloxy-2-(trifluoromethyl)hex-5-enoic acid (26.18 g, 96%) as pale brown oil. ¹H NMR (400 MHz, CDCl₃) δ 7.46-7.31 (m, 5H), 5.88-5.73 (m, 1H), 5.15-4.99 (m, 2H), 4.88 (d, J=10.3 Hz, 1H), 4.70 (d, J=10.3 Hz, 1H), 2.37-2.12 (m, 4H) ppm. ¹⁹F NMR (377 MHz, CDCl₃) δ −71.63 (br s, 3F) ppm. ESI-MS m/z calc. 288.0973, found 287.0 (M−1); Retention time: 2.15 minutes. LCMS Method: Kinetex Polar C₁₈ 3.0×50 mm 2.6 μm, 3 min, 5-95% acetonitrile in H₂O (0.1% formic acid) 1.2 mL/min.

### Intermediate 5: Preparation of (2R)-2-benzyloxy-2-(trifluoromethyl)hex-5-enehydrazide

### Step 1: tert-Butyl N-[[(2R)-2-benzyloxy-2-(trifluoromethyl)hex-5-enoyl]amino]carbamate

To a solution of (2R)-2-benzyloxy-2-(trifluoromethyl)hex-5-enoic acid (365 g, 1.266 mol) in DMF (2 L) was added HATU (612 g, 1.610 mol) and DIEA (450 mL, 2.584 mol) and the mixture was stirred at ambient temperature for 10 min. To the mixture was added tert-butyl N-aminocarbamate (200 g, 1.513 mol) (slight exotherm upon addition) and the mixture was stirred at ambient temperature for 16 h. The reaction was poured into ice water (5 L). The resultant precipitate was collected by filtration and washed with water. The solid was dissolved in EtOAc (2 L) and washed with brine. The organic phase was dried over MgSO₄, filtered and concentrated in vacuo. The oil was diluted with EtOAc (500 mL) followed by heptane (3 L) and stirred at ambient temperature for several hours affording a thick slurry. The slurry was diluted with additional heptane and filtered to collect fluffy white solid (343 g). The filtrate was concentrated and purification by silica gel chromatography (0-40% EtOAc/hexanes) provided tert-butyl N-[[(2R)-2-benzyloxy-2-(trifluoromethyl)hex-5-enoyl]amino]carbamate (464 g, 91%, combined with product from crystallization). ESI-MS m/z calc. 402.17664, found 303.0 (M+1-Boc)⁺; Retention time: 2.68 minutes. Final purity was determined by reversed phase UPLC using an Acquity UPLC BEH C₁₈ column (50×2.1 mm, 1.7 μm particle) made by Waters (pn: 186002350) and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate =1.2 mL/min, injection volume=1.5 μL, and column temperature=60° C.

### Step 2: (2R)-2-Benzyloxy-2-(trifluoromethyl)hex-5-enehydrazide

To a solution of tert-butyl N-[[(2R)-2-benzyloxy-2-(trifluoromethyl)hex-5-enoyl]amino]carbamate (464 g, 1.153 mol) in DCM (1.25 L) and was added HCl (925 mL of 4 M, 3.700 mol) and the mixture stirred at ambient temperature for 20 h. The mixture was concentrated in vacuo removing most of the DCM. The mixture was diluted with isopropyl acetate (1 L) and basified to pH=6 with NaOH (140 g of 50% w/w, 1.750 mol) in 1 L of ice water. The organic phase was separated and washed with 1 L of brine and the combined aqueous phases were extracted with isopropyl acetate (1 L). The combined organic phases were dried over MgSO₄, filtered and concentrated in vacuo affording a dark yellow oil of (2R)-2-benzyloxy-2-(trifluoromethyl)hex-5-enehydrazide (358 g, quant.). ¹H NMR (400 MHz, CDCl₃) δ 8.02 (s, 1H), 7.44-7.29 (m, 5H), 5.81 (ddt, J=16.8, 10.1, 6.4 Hz, 1H), 5.13-4.93 (m, 2H), 4.75 (dd, J=10.5, 1.5 Hz, 1H), 4.61 (d, J=10.5 Hz, 1H), 3.78 (s, 2H), 2.43 (ddd, J=14.3, 11.0, 5.9 Hz, 1H), 2.26-1.95 (m, 3H) ppm. ESI-MS m/z calc. 302.1242, found 303.0 (M+1)⁺; Retention time: 2.0 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate =1.2 mL/min, injection volume=1.5 μL, and column temperature=60° C. Intermediate 6: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

### Step 1: tert-Butyl N-[2-[[[(2R)-2-benzyloxy-2-(trifluoromethyl)hex-5-enoyl]amino]carbamoyl]-6-bromo-5-(trifluoromethyl)-3-pyridyl]carbamate

To a mixture of 6-bromo-3-(tert-butoxycarbonylamino)-5-(trifluoromethyl)pyridine-2-carboxylic acid (304 g, 789.3 mmol) and (2R)-2-benzyloxy-2-(trifluoromethyl)hex-5-enehydrazide (270 g, 893.2 mmol) in EtOAc (2.25 L) at ambient temperature was added DIEA (425 mL, 2.440 mol). To the mixture was slowly added T3P (622 g of 50% w/w, 977.4 mmol) using an ice-water bath to keep the temperature <35° C. (temperature rose to 34° C.) and the reaction mixture was stirred at ambient temperature for 18 h. Added additional DIEA (100 mL, 574.1 mmol) and T3P (95 g, 298.6 mmol) and stirred at ambient temperature for 2 days. Starting material was still observed and an additional T3P (252 g, 792 mmol) was added and stirred for 5 days. The reaction was quenched with the slow addition of water (2.5 L) and the mixture stirred for 30 min. The organic phase was separated, and the aqueous phase extracted with EtOAc (2 L). The combined organic phases were washed with brine, dried over MgSO₄, filtered and concentrated in vacuo. The crude product was dissolved in MTBE (300 mL) and diluted with heptane (3 L), the mixture stirred at ambient temperature for 12 h affording a light yellow slurry. The slurry was filtered, and the resultant solid was air dried for 2 h, then in vacuo at 40° C. for 48 h. The filtrate was concentrated in vacuo and purified by silica gel chromatography (0-20% EtOAc/hexanes) and combined with material obtained from crystallization providing tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (433 g, 82%). ¹H NMR (400 MHz, DMSO) δ 11.07 (s, 1H), 10.91 (s, 1H), 10.32 (s, 1H), 9.15 (s, 1H), 7.53-7.45 (m, 2H), 7.45-7.28 (m, 3H), 5.87 (ddt, J=17.0, 10.2, 5.1 Hz, 1H), 5.09 (dq, J=17.1, 1.3 Hz, 1H), 5.02 (dd, J=10.3, 1.9 Hz, 1H), 4.84 (q, J=11.3 Hz, 2H), 2.37-2.13 (m, 4H), 1.49 (s, 9H) ppm. ESI-MS m/z calc. 668.1069, found 669.0 (M+1)⁺; Retention time: 3.55 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 2: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

To a solution of tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (240 g, 358.5 mmol) in anhydrous acetonitrile (1.5 L) under nitrogen was added DIEA (230 mL, 1.320 mol) and the orange solution heated to 70° C. To the mixture was added p-toluenesulfonyl chloride (80.5 g, 422.2 mmol) in 3 equal portions over 1 h. The mixture was stirred at 70° C. for 9 h then additional p-toluenesulfonyl chloride (6.5 g, 34.09 mmol) was added. The mixture was stirred for a total of 24 h then allowed to cool to ambient temperature. Acetonitrile was removed in vacuo affording a dark orange oil which was diluted with EtOAc (1.5 L) and water (1.5 L). The organic phase was separated and washed with 500 mL of 1M HCl, 500 mL of brine, dried over MgSO₄, filtered and concentrated in vacuo. Purification by silica gel chromatography (0-20% EtOAc/hexanes) provided tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (200 g, 86%). ¹H NMR (400 MHz, DMSO) δ 10.11 (s, 1H), 9.10 (s, 1H), 7.55-7.48 (m, 2H), 7.47-7.28 (m, 3H), 5.87 (ddt, J=16.7, 10.2, 6.4 Hz, 1H), 5.11 (dt, J=17.2, 1.7 Hz, 1H), 5.01 (dt, J=10.2, 1.5 Hz, 1H), 4.74 (d, J=10.6 Hz, 1H), 4.65 (d, J=10.6 Hz, 1H), 2.55-2.42 (m, 2H), 2.30 (qd, J=11.3, 10.3, 6.9 Hz, 2H), 1.52 (s, 9H) ppm. ESI-MS m/z calc. 650.0963, found 650.0 (M+1)⁺; Retention time: 3.78 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 7: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

To a solution of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (222 g, 340.8 mmol) in MTBE (1.333 L) was added DIPEA (65.3 mL, 374.9 mmol) followed DMAP (2.09 g, 17.11 mmol). Added a solution of di-tert-butyl dicarbonate (111.6 g, 511.3 mmol) in MTBE (250 mL) over approx. 8 minutes, and the resulting mixture was stirred for additional 30 min. Added 1 L of water and separated the layers. The organic layer was washed with KHSO₄ (886 mL of 0.5 M, 443.0 mmol), 300 mL brine, dried with MgSO₄ and most (>95%) of the MTBE was evaporated by rotary evaporation at 45° C., leaving a thick oil. Added 1.125 L of heptane, spun in the 45° C. rotovap bath until dissolved, then evaporated out 325 mL of solvent by rotary evaporation. The rotovap bath temp was allowed to drop to room temperature and product started crystallizing out during the evaporation. Then put the flask in a −20° C. freezer overnight. The resultant solid was filtered and washed with cold heptane and dried at room temperature for 3 days to give tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (240.8 g, 94%). ¹H NMR (400 MHz, Chloroform-d) δ 7.95 (s, 1H), 7.52-7.45 (m, 2H), 7.44-7.36 (m, 2H), 7.36-7.29 (m, 1H), 5.83-5.67 (m, 1H), 5.08-5.00 (m, 1H), 5.00-4.94 (m, 1H), 4.79 (d, J=10.4 Hz, 1H), 4.64 (d, J=10.4 Hz, 1H), 2.57-2.26 (m, 3H), 2.26-2.12 (m, 1H), 1.41 (s, 18H) ppm. ESI-MS m/z calc. 750.14874, found 751.1 (M+1)⁺; Retention time: 3.76 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 8: Preparation of tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (280 g, 372.6 mmol) was dissolved in DMSO (1.82 L) (yellow solution) and treated with cesium acetate (215 g, 1.120 mol) under stirring at room temperature. The yellow suspension was heated at 80° C. for 5 h. The reaction mixture was cooled to room temperature and added to a stirred cold emulsion of water (5.5 L) with 1 kg ammonium chloride dissolved in it and a 1:1 mixture of MTBE and heptane (2 L) (in 20 L). The phases were separated and the organic phase washed water (3 ×3 L) and with brine (1×2.5 L). The organic phase was dried with MgSO₄, filtered and concentrated under reduced pressure. The resultant yellow solution was diluted with heptane (˜1 L) and seeded with tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate and stirred on the rotavap at 100 mbar pressure at room temperature for 1.5 h. The solid mass was stirred mechanically for 2 h at room temperature, resultant thick fine suspension was filtered, washed with dry ice cold heptane and dried under vacuum at 45° C. with a nitrogen bleed for 16 h to give tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (220 g, 85%) as an off white solid. ¹H NMR (400 MHz, DMSO-d₆) δ 13.28 (s, 1H), 8.43 (s, 1H), 7.58-7.26 (m, 5H), 5.85 (ddt, J=16.8, 10.3, 6.5 Hz, 1H), 5.10 (dq, J=17.2, 1.6 Hz, 1H), 5.01 (dq, J=10.2, 1.3 Hz, 1H), 4.76 (d, J=11.0 Hz, 1H), 4.65 (d, J=11.0 Hz, 1H), 2.55 (dd, J=9.6, 5.2 Hz, 2H), 2.23 (td, J=13.2, 10.0, 5.7 Hz, 2H), 1.27 (d, J=3.8 Hz, 18H) ppm. ESI-MS m/z calc. 688.23315, found 689.0 (M+1)⁺; Retention time: 3.32 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B =CH₃CN (0.035% CF₃CO₂H). Flow rate=1.2 mL/min, injection volume=1.5 μL, and column temperature=60° C.

### C. Preparation of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

### Step 1: tert-Butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate

Dissolved tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (159.3 g, 231.3 mmol) and triphenylphosphine (72.9 g, 277.9 mmol) in toluene (1 L), then added (2S)-pent-4-en-2-ol (28.7 mL, 278.9 mmol). Heated this mixture to 45° C., then added DIAD (58.3 mL, 296.1 mmol) (exotherm) slowly over 40 min. For the next approximately 2 h, the mixture was cooled to room temperature. During this cooling period, after the first 10 minutes, triphenylphosphine (6.07 g, 23.14 mmol) was added. After a further 1 h, additional triphenylphosphine (3.04 g, 11.59 mmol) was added. After a further 23 min, DIAD (2.24 mL, 11.57 mmol) was added. After the ˜2 h cooling to room temperature period, the mixture was cooled to 15° C., and seed crystals of DIAD-triphenylphosphine oxide complex were added which caused precipitation to occur, then added 1000 mL heptane. Stored the mixture at −20° C. for 3 days. Filtered out and discarded the precipitate and concentrated the filtrate to give a red residue/oil. Dissolved the residue in 613 mL heptane at 45° C., then cooled to 0° C., seeded with DIAD-triphenylphosphine oxide complex, stirred at 0° C. for 30 min, then filtered the solution. The filtrate was concentrated to a smaller volume, then loaded onto a 1.5 kg silica gel column (column volume=2400 mL, flow rate=600 mL/min). Ran a gradient of 1% to 6% EtOAc in hexanes over 32 minutes (8 column volumes), then held at 6% EtOAc in hexanes until the product finished eluting which gave tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (163.5 g, 93%). ¹H NMR (400 MHz, Chloroform-d) δ 7.82 (s, 1H), 7.43-7.27 (m, 5H), 5.88-5.69 (m, 2H), 5.35 (h, J=6.2 Hz, 1H), 5.16-4.94 (m, 4H), 4.81 (d, J=10.7 Hz, 1H), 4.63 (d, J=10.7 Hz, 1H), 2.58-2.15 (m, 6H), 1.42 (s, 18H), 1.36 (d, J=6.2 Hz, 3H) ppm. ESI-MS m/z calc. 756.2958, found 757.3 (M+1)⁺; Retention time: 4.0 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 2: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture)

The following reaction was run, split equally between two, 12 L reaction flasks run in parallel. Mechanical stirring was employed, and reactions were subjected to a constant nitrogen gas purge using a course porosity gas dispersion tube. To each flask was added tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (54 g, 71.36 mmol in each flask) dissolved in DCE (8 L in each flask) and both flasks were strongly purged with nitrogen at room temperature. Both flasks were heated to 62° C. and Grubbs 1^{st} Generation Catalyst (9 g, 10.94 mmol in each flask) was added to each reaction and stirred at 400 rpm while setting an internal temperature control to 75° C. with strong nitrogen purging (both reactions reached −75° C. after approximately 20 min). After 5 h 15 min, the internal temperature control was set to 45° C. After approximately 2 h, 2-sulfanylpyridine-3-carboxylic acid (11 g, 70.89 mmol in each flask) was added to each flask followed by triethylamine (10 mL, 71.75 mmol in each flask). On completion of addition, the nitrogen purge was turned off and both reaction flasks were stirred at 45° C. open to air overnight. The reactions were then removed from heat and 130 g of silica gel was added to each reaction and each was stirred at room temperature. After approximately 2 h, the green mixtures were combined and filtered over Celite then concentrated by rotary evaporation at 43° C. The obtained residue was dissolved in dichloromethane/heptane 1:1 (400 mL) and the formed orange solid was removed by filtration. The greenish mother liquor was evaporated to give 115.5 g of a green foam. Dissolved this material in 500 mL of 1:1 dichloromethane/hexanes then loaded onto a 3 kg silica gel column (column volume=4800 mL, flow rate=900 mL/min). Ran a gradient of 2% to 9% EtOAc in hexanes over 43 minutes (8 column volumes), then ran at 9% EtOAc until the product finished eluting giving 77.8 g of impure product. This material was co-evaporated with methanol (˜500 mL) then diluted with methanol (200 mL) to give 234.5 g of a methanolic solution, which was halved and each half was purified by reverse phase chromatography (3.8 kg C₁₈ column, column volume=3300 mL, flow rate=375 mL/min, loaded as solution in methanol). Ran the column at 55% acetonitrile for ˜5 minutes (0.5 column volumes), then at a gradient of 55% to 100% acetonitrile in water over ˜170 minutes (19-20 column volumes), then held at 100% acetonitrile until the product and impurities finished eluting. Clean product fractions from both columns were combined and concentrated by rotary evaporation then transferred with ethanol into 5 L flask, evaporated and carefully dried (becomes a foam) to give as a mixture of olefin isomers, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (55.5 g, 53%). ESI-MS m/z calc. 728.26447, found 729.0 (M+1)⁺; Retention time: 3.82 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 3: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (11.7 g, 16.06 mmol) was dissolved in stirring ethanol (230 mL) and cycled the flask 3 times vacuum/nitrogen and treated with 10% Pd/C (50% water wet, 2.2 g of 5% w/w, 1.034 mmol). The mixture was cycled 3 times between vacuum/nitrogen and 3 times between vacuum/hydrogen. The mixture was then stirred strongly under hydrogen (balloon) for 7.5 h. The catalyst was removed by filtration, replaced with fresh 10% Pd/C (50% water wet, 2.2 g of 5% w/w, 1.034 mmol) and stirred vigorously under hydrogen (balloon) overnight. Then, the catalyst was removed again by filtration, the filtrate evaporated and the residue (11.3 g, 1 g set aside) was dissolved in ethanol (230 mL) charged with fresh 10% Pd/C (50% water wet, 2.2 g of 5% w/w, 1.034 mmol) and stirred vigorously under hydrogen (balloon) for 6 h, recharged again with fresh 10% Pd/C (50% water wet, 2.2 g of 5% w/w, 1.034 mmol) and stirred vigorously under hydrogen (balloon) overnight. The catalyst was removed by filtration and the filtrate was evaporated (10 g of residue obtained). This crude material (10 g+1 g set aside above) was purified by silica gel chromatography (330 g column, liquid load in dichloromethane) with a linear gradient of 0% to 15% ethyl acetate in hexane until the product eluted followed by 15% to 100% ethyl acetate in hexane to giving, as a colorless foam, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (9.1 g, 78%). ESI-MS m/z calc. 730.2801, found 731.0 (M+1)⁺; Retention time: 3.89 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 4: (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (8.6 g, 11.77 mmol) was dissolved in ethanol (172 mL) then the flask was cycled 3 times between vacuum/nitrogen. Treated the mixture with 10% Pd/C (50% water wet, 1.8 g of 5% w/w, 0.8457 mmol) then cycled 3 times between vacuum/nitrogen and 3 times between vacuum/hydrogen and then stirred vigorously under hydrogen (balloon) at room temperature for 18 h. The mixture was cycled 3 times between vacuum/nitrogen, filtered over Celite washing with ethanol and then the filtrate was evaporated to give 7.3 g of tert-butyl N-tert-butoxycarbonyl-N-[(6R,12R)-6-hydroxy-12-methyl-6,15-bis(trifluoromethyl)-13,19-dioxa-3,4,18-triazatricyclo[12.3.1.12,5] nonadeca-1(18),2,4,14,16-pentaen-17-yl]carbamate an off-white solid. 1H NMR and MS confirmed the expected product. CFTR modulatory activity was confirmed using a standard Ussing Chamber Assay for CFTR potentiator activity.

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A compound of Formula I: 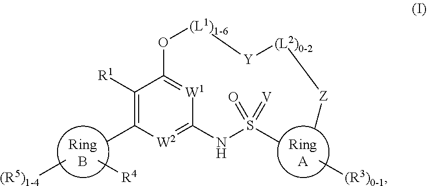
a tautomer thereof, a deuterated derivative of the compound or tautomer, or a pharmaceutically acceptable salt of any of the foregoing, wherein:
Ring A is selected from:
C₆-C₁₀ aryl,
C₃-C₁₀ cycloalkyl,
3- to 10-membered heterocyclyl, and
5- to 10-membered heteroaryl;
Ring B is selected from:
C₆-C₁₀ aryl,
C₃-C₁₀ cycloalkyl,
3- to 10-membered heterocyclyl, and
5- to 10-membered heteroaryl;
V is selected from O and NH;
W¹ is selected from N and CH;
W² is selected from N and CH; provided that at least one of W¹ and W² is N;
Y is selected from O, NR^{YN}, and C(R^{YC})₂;
Z is selected from O, NR^{ZN}, and C(R^{ZC})₂, provided that when L² is absent, either Y is C(R^{YC})₂ or Z is C(R^{ZC})₂;
each L¹ is independently selected from C(R^{L1})₂ and 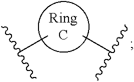
each L² is independently selected from C(R^{L2})₂;
Ring C is selected from C₆-C₁₀ aryl optionally substituted with 1-3 groups independently selected from:
halogen,
C₁-C₆ alkyl, and
N(R^{N})₂;
R¹ is selected from:
halogen,
cyano,
C₁-C₆ alkyl optionally substituted with 1-3 groups independently selected from hydroxyl, oxo, and N(R^{N})₂,
C₁-C₆ alkoxy,
C₁-C₆ fluoroalkyl,
C₆-C₁₀ aryl optionally substituted with 1-3 groups independently selected from C₁-C₆ alkoxy,
3- to 10-membered heterocyclyl optionally substituted with 1-3 groups independently selected from R^{N}, and
5- to 10-membered heteroaryl optionally substituted with 1-3 groups independently selected from C₁-C₆ alkyl;
each R³ is independently selected from:
halogen,
C₁-C₆ alkyl,
C₁-C₆ alkoxy,
C₃-C₁₀ cycloalkyl,
C₆-C₁₀ aryl optionally substituted with 1-3 groups independently selected from C₁-C₆ alkyl, and
3- to 10-membered heterocyclyl;
R⁴ is selected from hydrogen and C₁-C₆ alkyl;
each R⁵ is independently selected from:
hydrogen,
halogen,
hydroxyl,
N(R^{N})₂,
SO-Me,
CH═C(R^{LC})₂, wherein both R^{LC} are taken together to form a C₃-C₁₀ cycloalkyl,
C₁-C₆ alkyl optionally substituted with 1-3 groups independently selected from:
hydroxyl,
C₁-C₆ alkoxy optionally substituted with 1-3 groups independently selected from C₁-C₆ alkoxy and C₆-C₁₀ aryl,
C₃-C₁₀ cycloalkyl,
—(O)₀₋₁—(C₆-C₁₀ aryl) optionally substituted with 1-3 groups independently selected from C₁-C₆ alkyl and C₁-C₆ alkoxy,
3- to 10-membered heterocyclyl, and
N(R^{N})₂,
C₁-C₆ alkoxy optionally substituted with 1-3 groups independently selected from:
halogen,
C₆-C₁₀ aryl, and
C₃-C₁₀ cycloalkyl optionally substituted with 1-3 groups independently selected from C₁-C₆ fluoroalkyl,
C₁-C₆ fluoroalkyl,
C₃-C₁₀ cycloalkyl,
C₆-C₁₀ aryl, and
3- to 10-membered heterocyclyl;
each R^{YN} and R^{ZN} is independently selected from:
hydrogen,
C₁-C₉ alkyl optionally substituted with 1-3 groups independently selected from:
hydroxyl,
oxo,
cyano,
C₁-C₆ alkoxy optionally substituted with 1-3 groups independently selected from halogen and C₁-C₆ alkoxy,
N(R^{N})₂,
SO₂Me,
C₃-C₁₀ cycloalkyl optionally substituted with 1-3 groups independently selected from:
hydroxyl,
C₁-C₆ alkyl optionally substituted with 1-3 groups independently selected from hydroxyl, oxo, C₁-C₆ alkoxy, C₆-C₁₀ aryl, and N(R^{N})₂,
C₁-C₆ fluoroalkyl,
C₁-C₆ alkoxy, and
COOH,
N(R^{N})₂,
C₆-C₁₀ aryl, and
3- to 10-membered heterocyclyl optionally substituted with 1-3 groups independently selected from oxo and C₁-C₆ alkyl,
C₆-C₁₀ aryl optionally substituted with 1-3 groups independently selected from:
halogen,
hydroxyl,
cyano,
SiMe₃,
SO₂Me,
SF₅,
N(R^{N})₂,
P(O)Me₂,
—(O)₀₋₁—(C₃-C₁₀ cycloalkyl) optionally substituted with 1-3 groups independently selected from C₁-C₆ fluoroalkyl,
C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
C₁-C₆ alkoxy optionally substituted with 1-3 groups independently selected from hydroxyl, oxo, N(R^{N})₂, and C₆-C₁₀ aryl,
C₁-C₆ fluoroalkyl,
3- to 10-membered heterocyclyl optionally substituted with 1-3 groups independently selected from C₁-C₆ alkyl,
—(O)₀₋₁—(C₆-C₁₀ aryl), and
—(O)₀₋₁-(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
3- to 10-membered heterocyclyl optionally substituted with 1-4 groups independently selected from:
hydroxyl,
oxo,
N(R^{N})₂,
C₁-C₆ alkyl (optionally substituted with 1-3 groups independently selected from oxo and C₁-C₆ alkoxy),
C₁-C₆ alkoxy,
C₁-C₆ fluoroalkyl,
C₆-C₁₀ aryl optionally substituted with 1-3 groups independently selected from halogen, and
5- to 10-membered heteroaryl, and
5- to 10-membered heteroaryl optionally substituted with 1-3 groups independently selected from:
hydroxyl,
cyano,
oxo,
halogen,
B(OH)₂,
N(R^{N})₂,
C₁-C₆ alkyl optionally substituted with 1-3 groups independently selected from hydroxyl, oxo, C₁-C₆ alkoxy (optionally substituted with 1-3-SiMe₃), and N(R^{N})₂,
C₁-C₆ alkoxy optionally substituted with 1-3 groups independently selected from hydroxyl, oxo, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from halogen and C₁-C₆ alkyl,
—(O)₀₋₁—(C₆-C₁₀ aryl),
—(O)₀₋₁-(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl) and
5- to 10-membered heteroaryl optionally substituted with 1-4 groups independently selected from C₁-C₆ alkyl and C₃-C₁₀ cycloalkyl,
C₁-C₆ fluoroalkyl,
C₃-C₁₀ cycloalkyl optionally substituted with 1-3 groups independently selected from:
hydroxyl,
oxo,
halogen,
cyano,
N(R^{N})₂,
C₁-C₆ alkyl optionally substituted with 1-3 groups independently selected from:
hydroxyl,
oxo,
N(R^{N})₂,
C₁-C₆ alkoxy, and
C₆-C₁₀ aryl,
C₁-C₆ alkoxy optionally substituted with 1-3 groups independently selected from halogen, oxo, C₆-C₁₀ aryl, and N(R^{N})₂,
halogen,
C₃-C₁₀ cycloalkyl,
3- to 10-member heterocyclyl optionally substituted with 1-3 groups independently selected from C₁-C₆ alkyl, and
5- to 10-membered heteroaryl optionally substituted with 1-3 groups independently selected from:
hydroxyl,
cyano,
oxo,
halogen,
N(R^{N})₂,
C₁-C₆ alkyl optionally substituted with 1-3 groups independently selected from hydroxyl, oxo, C₁-C₆ alkoxy, and N(R^{N})₂,
C₁-C₆ alkoxy optionally substituted with 1-3 groups independently selected from hydroxyl, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
C₆-C₁₀ aryl, and
3- to 10-membered heterocyclyl optionally substituted with 1-3 groups independently selected from C₁-C₆ alkyl,
C₆-C₁₀ aryl,
3- to 10-membered heterocyclyl optionally substituted with 1-3 groups independently selected from:
oxo,
C₁-C₆ alkyl optionally substituted with 1-3 groups independently selected from:
oxo,
hydroxyl,
N(R^{N})₂,
C₁-C₆ alkoxy optionally substituted with 1-3 groups independently selected from halogen and C₆-C₁₀ aryl, and
—(O)₀₋₁—(C₃-C₁₀ cycloalkyl),
C₁-C₆ fluoroalkyl,
C₃-C₁₀ cycloalkyl optionally substituted with 1-3 groups independently selected from halogen, and
3- to 10-membered heterocyclyl,
5- to 10-membered heteroaryl optionally substituted with 1-3 groups independently selected from:
halogen,
C₁-C₆ alkyl optionally substituted with 1-3 groups independently selected from oxo, C₁-C₆ alkoxy, and N(R^{N})₂, and
3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
R^{F},
each R^{YC} and R^{ZC} is independently selected from:
hydrogen,
C₁-C₆ alkyl optionally substituted with 1-3 groups independently selected from C₆-C₁₀ aryl (optionally substituted with 1-3 groups independently selected from C₁-C₆ alkyl),
C₆-C₁₀ aryl optionally substituted with 1-3 groups independently selected from C₁-C₆ alkyl, and
R^{F},
or two R^{YC} are taken together to form an oxo group;
or two R^{ZC} are taken together to form an oxo group;
each R^{L1} is independently selected from:
hydrogen,
N(R^{N})₂, provided that two N(R^{N})₂ are not bonded to the same carbon,
C₁-C₉ alkyl optionally substituted with 1-3 groups independently selected from:
halogen,
hydroxyl,
oxo,
N(R^{N})₂,
C₁-C₆ alkoxy optionally substituted with 1-3 groups independently selected from C₆-C₁₀ aryl,
C₃-C₁₀ cycloalkyl optionally substituted with 1-3 groups independently selected from halogen and C₁-C₆ fluoroalkyl,
C₆-C₁₀ aryl optionally substituted with 1-3 groups independently selected from C₁-C₆ alkyl, and
3- to 10-membered heterocyclyl optionally substituted with 1-3 groups independently selected from C₁-C₆ alkyl (optionally substituted with 1-3 groups independently selected from hydroxyl and oxo),
C₃-C₁₀ cycloalkyl,
C₆-C₁₀ aryl optionally substituted with 1-4 groups independently selected from:
halogen,
cyano,
SiMe₃,
POMe₂,
C₁-C₇ alkyl optionally substituted with 1-3 groups independently selected from:
hydroxyl,
oxo,
cyano,
SiMe₃,
N(R^{N})₂, and
C₃-C₁₀ cycloalkyl optionally substituted with 1-3 groups independently selected from C₁-C₆ fluoroalkyl,
C₁-C₆ alkoxy optionally substituted with 1-3 groups independently selected from:
C₃-C₁₀ cycloalkyl optionally substituted with 1-3 groups independently selected from C₁-C₆ fluoroalkyl, and
C₁-C₆ alkoxy,
C₁-C₆ fluoroalkyl,
C₃-C₁₀ cycloalkyl optionally substituted with 1-3 groups independently selected from C₁-C₆ alkyl and C₁-C₆ fluoroalkyl,
C₆-C₁₀ aryl,
3- to 10-membered heterocyclyl optionally substituted with 1-3 groups independently selected from C₁-C₆ alkyl, and
5- to 10-membered heteroaryl,
3- to 10-membered heterocyclyl optionally substituted with 1-3 groups independently selected from:
C₁-C₆ alkyl optionally substituted with 1-3 groups independently selected from:
oxo, and
C₁-C₆ alkoxy,
5- to 10-membered heteroaryl optionally substituted with 1-3 groups independently selected from:
C₁-C₆ alkyl optionally substituted with 1-3 groups independently selected from:
C₃-C₁₀ cycloalkyl optionally substituted with 1-3 groups independently selected from C₁-C₆ fluoroalkyl, and
C₆-C₁₀ aryl optionally substituted with 1-3 groups independently selected from C₁-C₆ alkyl, and
R^{F};
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
hydrogen,
C₁-C₈ alkyl optionally substituted with 1-3 groups independently selected from:
oxo,
halogen,
hydroxyl,
NH₂,
NHMe,
NMe₂,
C₁-C₆ alkoxy optionally substituted with 1-3 groups independently selected from C₆-C₁₀ aryl,
—(O)₀₋₁—(C₃-C₁₀ cycloalkyl),
C₆-C₁₀ aryl optionally substituted with 1-3 groups independently selected from halogen and C₁-C₆ alkyl, and
3- to 14-membered heterocyclyl optionally substituted with 1-4 groups independently selected from oxo and C₁-C₆ alkyl, and
5- to 14-membered heteroaryl optionally substituted with 1-4 groups independently selected from oxo and C₁-C₆ alkyl,
C₃-C₁₀ cycloalkyl optionally substituted with 1-3 groups independently selected from:
hydroxyl,
NH₂, and
NHMe,
C₁-C₆ alkyl optionally substituted with 1-3 groups independently selected from hydroxyl, and
C₆-C₁₀ aryl, and
3- to 10-membered heterocyclyl;
or two R^{N} on the same nitrogen atom are taken together with the nitrogen to which they are bonded to form a 3- to 10-membered heterocyclyl optionally substituted with 1-3 groups selected from:
hydroxyl,
oxo,
cyano,
C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
C₁-C₆ alkoxy, and
C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
C₃-C₁₀ cycloalkyl optionally substituted with 1-3 groups independently selected from C₁-C₆ alkyl,
C₆-C₁₀ aryl optionally substituted with 1-3 groups independently selected from:
halogen,
C₁-C₆ alkyl,
N(R^{N})₂, and
3- to 10-membered heterocyclyl optionally substituted with 1-3 groups independently selected from hydroxyl,
3- to 11-membered heterocyclyl optionally substituted with 1-3 groups independently selected from:
oxo,
N(R^{N})₂,
C₁-C₉ alkyl optionally substituted with 1-4 groups independently selected from:
oxo,
halogen,
hydroxyl,
N(R^{N})₂,
SO₂-(C₁-C₆ alkyl),
C₁-C₆ alkoxy optionally substituted with 1-3 groups independently selected from halogen, C₆-C₁₀ aryl,
C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
—(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N}, —O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
C₃-C₁₂ cycloalkyl optionally substituted with 1-4 groups independently selected from halogen, C₁-C₆ alkyl, and C₁-C₆ fluoroalkyl,
C₆-C₁₀ aryl,
3- to 10-membered heterocyclyl, and
5- to 10-membered heteroaryl optionally substituted with 1-3 groups independently selected from C₁-C₆ alkoxy and C₁-C₆ fluoroalkyl, and
5- to 12-membered heteroaryl optionally substituted with 1-3 groups independently selected from C₁-C₆ alkyl and C₁-C₆ fluoroalkyl.

2. A compound of Formula Ia: 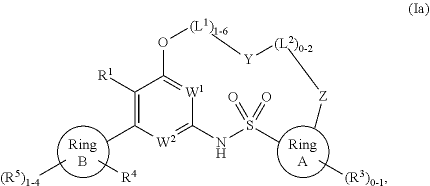
a tautomer thereof, a deuterated derivative of the compound or tautomer, or a pharmaceutically acceptable salt of any of the foregoing, wherein Ring A, Ring B, W¹, W², Y, Z, L¹, L², R¹, R³, R⁴, and R⁵ are defined as according to claim 1.

3. A compound of Formula IIa: 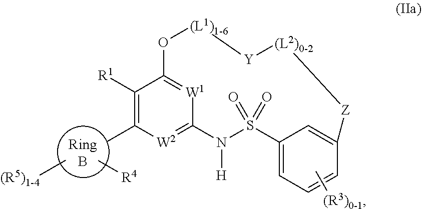
a tautomer thereof, a deuterated derivative of the compound or tautomer, or a pharmaceutically acceptable salt of any of the foregoing, wherein Ring B, W¹, W², X, Y, Z, L¹, L², R¹, R³, R⁴, and R⁵ are defined as according to claim 1.

4. A compound of Formula IIb: 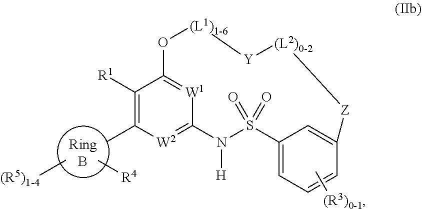
a tautomer thereof, a deuterated derivative of the compound or tautomer, or a pharmaceutically acceptable salt of any of the foregoing, wherein Ring A, W¹, W², Y, Z, L¹, L², R¹, R³, R⁴, and R⁵ are defined as according to claim 1.

5. A compound of Formula III: 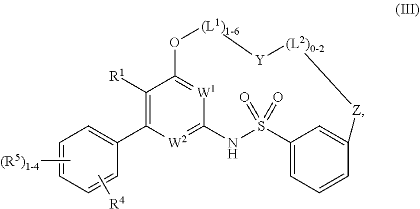
a tautomer thereof, a deuterated derivative of the compound or tautomer, or a pharmaceutically acceptable salt of any of the foregoing, wherein W¹, W², Y, Z, L¹, L², R¹, R⁴, and R⁵ are defined as according to claim 1.

6. A compound of Formula IV: 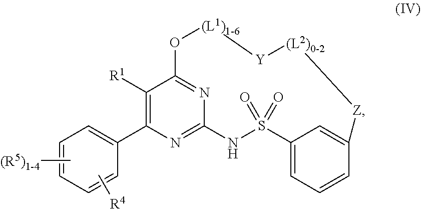
a tautomer thereof, a deuterated derivative of the compound or tautomer, or a pharmaceutically acceptable salt of any of the foregoing, wherein Y, Z, L¹, L², R¹, R⁴, and R⁵ are defined as according to claim 1.

7. A compound of Formula V: 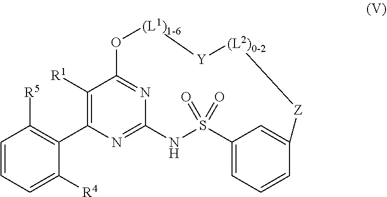
a tautomer thereof, a deuterated derivative of the compound or tautomer, or a pharmaceutically acceptable salt of any of the foregoing, wherein Y, Z, L¹, L², R¹, R⁴ and R⁵ are defined as according to claim 1.

8. A compound of Formula VIa: 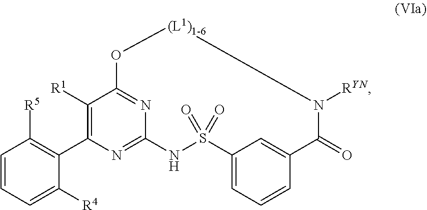
a tautomer thereof, a deuterated derivative of the compound or tautomer, or a pharmaceutically acceptable salt of any of the foregoing, wherein L¹, R¹, R⁴, R⁵, and R^{YN} are defined as according to claim 1.

9. A compound of Formula VIb: 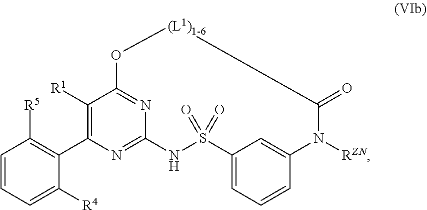
a tautomer thereof, a deuterated derivative of the compound or tautomer, or a pharmaceutically acceptable salt of any of the foregoing, wherein L¹, R¹, R⁴, R⁵, and R^{ZN} are defined as according to claim 1.

10. The compound, tautomer, deuterated derivative, or pharmaceutically acceptable salt according to any one of claims 1 to 9, selected from compounds of compounds of any one of Formulae I, Ia, IIa, IIb, III, IV, V, VIa, and VIb, and tautomers thereof and deuterated derivatives of those compounds and tautomers and pharmaceutically acceptable salts of any of the foregoing.

11. The compound, tautomer, deuterated derivative, or pharmaceutically acceptable salt according to any one of claims 1 to 10, selected from Compounds 1-320 (Tables 3-7, 9, 11, 13, 15, 17, 20, and 21), Compounds 321-330 (Table 19), Compounds 331-364 (Tables 22-24), and Compounds 365-369 (Tables 25-27), tautomers thereof, deuterated derivatives thereof of those compounds and tautomers, and pharmaceutically acceptable salts of any of the foregoing.

12. A pharmaceutical composition comprising the compound, tautomer, deuterated derivative, or pharmaceutically acceptable salt according to any one of claims 1 to 11, and a pharmaceutically acceptable carrier.

13. The pharmaceutical composition of claim 12, further comprising one or more additional therapeutic agents.

14. The pharmaceutical composition of claim 13, wherein the one or more additional therapeutics agents is selected from one or more CFTR modulating agents.

15. The pharmaceutical composition of claim 14, wherein the one or more CFTR modulating agents is selected from potentiators.

16. The pharmaceutical composition of claim 14, wherein the one or more CFTR modulating agents is selected from CFTR correctors.

17. The pharmaceutical composition of claim 14, wherein the one or more CFTR modulating agents includes both a potentiator and a corrector.

18. The pharmaceutical composition of claim 13, wherein the one or more additional therapeutic agents are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

19. A method of treating cystic fibrosis comprising administering to a patient in need thereof the compound, tautomer, deuterated derivative, or pharmaceutically acceptable salt according to any one of claims 1 to 11, or a pharmaceutical composition according to any one of claims 12 to 18.

20. The method of claim 19, further comprising administering to the patient one or more additional therapeutic agents prior to, concurrent with, or subsequent to the compound, tautomer, deuterated derivative, or pharmaceutically acceptable salt according to any one of claims 1 to 11, or the pharmaceutical composition according to any one of claim 12.

21. The method of claim 20, wherein the one or more additional therapeutic agents is a compound selected from CFTR modulating agents.

22. The method of claim 21, wherein the one or more CFTR modulating agents is selected from potentiators.

23. The method of claim 21, wherein the one or more CFTR modulating agents is selected from CFTR correctors.

24. The method of claim 21, wherein the one or more CFTR modulating agents includes both a potentiator and a corrector.

25. The method of claim 21, wherein the one or more CFTR modulating agents si selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

26. The compound, tautomer, deuterated derivative, or pharmaceutically acceptable salt according to any one of claims 1 to 11, or the pharmaceutical composition according to any one of claims 12 to 18 for use in the treatment of cystic fibrosis.

27. The compound, tautomer, deuterated derivative, or pharmaceutically acceptable salt according to any one of claims 1 to 11, or the pharmaceutical composition according to any one of claims 12 to 18 for use in the manufacture of a medicament for the treatment of cystic fibrosis.
